(19) United States
(12) Patent Application Publication
(10) Pub. No.: US 2026/0092057 A1
(43) Pub. Date: Apr. 2, 2026

(54) BCL-XL/BCL-2 DEGRADERS AND USES THEREOF

(71) Applicant: Kymera Therapeutics, Inc., Watertown, MA (US)

(72) Inventors: Huijun Dong, Watertown, MA (US); Bin Yang, Watertown, MA (US); Lewis Dale Pennington, Watertown, MA (US); Eamon Comer, Watertown, MA (US); Melissa Ford, Watertown, MA (US); Robert Aversa, Watertown, MA (US); Matthew M. Weiss, Watertown, MA (US); Xiao Zhu, Watertown, MA (US); Thijs Beuming, Cambridge, MA (US)

(21) Appl. No.: 18/864,802

(22) PCT Filed: May 12, 2023
(86) PCT No.: PCT/US2023/022120

§ 371 Date: Nov. 11, 2024

Related U.S. Application Data
(60) Provisional application No. 63/364,589, filed on May 12, 2022.
(60) Provisional application No. 63/375,819, filed on Sep. 15, 2022.
(60) Provisional application No. 63/380,918, filed on Oct. 25, 2022.

(51) Int. Cl. C07D 417/12; A61K 31/496; A61K 31/635; A61P 35/00; C07D 207/16; C07D 401/12; C07D 417/14; C07D 471/04; C07D 471/08; C07D 471/10; C07D 487/04; C07D 487/08; C07D 487/10; C07D 519/00

(52) U.S. Cl. CPC C07D 417/12; A61K 31/496; A61K 31/635; A61P 35/00; C07D 207/16; C07D 401/12; C07D 417/14; C07D 471/04; C07D 471/08; C07D 471/10; C07D 487/04; C07D 487/08; C07D 487/10; C07D 519/00

(57) ABSTRACT

The present invention provides compounds, compositions thereof, and methods of using the same.

### CROSS-REFERENCE TO RELATED APPLICATIONS

This application claims the benefit of priority to U.S. Provisional Appl. No. 63/364,589, filed May 12, 2022, U.S. Provisional Appl. No. 63/375,819, filed Sep. 15, 2022, and U.S. Provisional Appl. No. 63/380,918, filed Oct. 25, 2022, the entirety of which is herein incorporated by reference.

### TECHNICAL FIELD OF THE INVENTION

The present invention relates to compounds and methods useful for the modulation of B-cell lymphoma-extra large (BCL-XL) and B-cell lymphoma-2 (BCL-2) via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s. U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Berndsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer, 2014, 14, 233-347) titled “Roles of F-box proteins in cancer.”

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway. DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e., abnormal or accelerated degradation of the protein target.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth J S Jr., Chembiochem, 2005, 6(1):40-46).

The BCL-2 (B-cell lymphoma-2) family of proteins is a group of regulator proteins that plays a central role in regulating cell death by either inducing (pro-apoptotic) or inhibiting (anti-apoptotic) apoptosis. The anti-apoptotic BCL-2 family of proteins, such as BCL-2, BCL-XL, BCL-W, and MCL-1, are attractive target for the development of novel anti-cancer agents.

There is an ongoing need in the art for effective treatments for disease, especially cancer. As such, small molecule therapeutic agents that leverage E3 ligase mediated protein degradation to cancer associated proteins such as B-cell lymphoma-extra large (BCL-XL) and B-cell lymphoma-2 (BCL-2) hold promise as therapeutic agents. Accordingly, there remains a need to find compounds that are BCL-XL and BCL-2 degraders useful as therapeutic agents.

### SUMMARY OF THE INVENTION

The present application relates novel bifunctional compounds, which function to recruit BCL-XL and BCL-2 protein to E3 ubiquitin ligase for degradation, and methods of preparation and uses thereof. In particular, the present disclosure provides bifunctional compounds, which find utility as modulators of targeted ubiquitination of BCL-XL and BCL-2 protein, which are then degraded and/or otherwise inhibited by the bifunctional compounds as described herein. Also provided are monovalent compounds, which find utility as inducers of targeted ubiquitination of BCL-XL and BCL-2 protein, which are then degraded and/or otherwise inhibited by the monovalent compounds as described herein. An advantage of the compounds provided herein is that a broad range of pharmacological activities is possible, consistent with the degradation/inhibition of BCL-XL and BCL-2 protein. In addition, the description provides methods of using an effective amount of the compounds as described herein for the treatment or amelioration of a disease condition, such as cancer.

The present application further relates to targeted degradation of BCL-XL and BCL-2 protein through the use of bifunctional molecules, including bifunctional molecules that link a cereblon-binding moiety to a ligand that binds BCL-XL and BCL-2 protein.

It has now been found that compounds of this invention, and pharmaceutically acceptable compositions thereof, are effective as degraders of BCL-XL and BCL-2 protein. Such compounds have the general formula I:

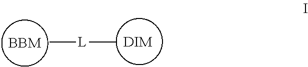

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of signaling pathways implicating BCL-XL and BCL-2 protein. Such diseases, disorders, or conditions include those described herein.

Compounds provided by this invention are also useful for the study of BCL-XL and BCL-2 protein in biological and pathological phenomena; the study of intracellular signal transduction pathways occurring in bodily tissues; and the comparative evaluation of new BCL-XL and BCL-2 inhibitors or BCL-XL and BCL-2 degraders or other regulators of cell cycling, metastasis, angiogenesis, and immune cell evasion, in vitro or in vivo.

### DETAILED DESCRIPTION OF CERTAIN EMBODIMENTS

### 1. General Description of Certain Embodiments of the Invention

Compounds of the present invention, and compositions thereof, are useful as degraders and/or inhibitors of BCL-XL and BCL-2 protein.

In certain embodiments, the present invention provides a compound of formula I:

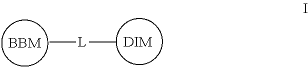

or a pharmaceutically acceptable salt thereof, wherein:
BBM is a BCL-XL and BCL-2 binding moiety capable of binding to BCL-XL and BCL-2;
L is a bivalent moiety that connects BBM to DIM; and
DIM is a degradation inducing moiety selected from an E3 ubiquitin ligase binding moeity (LBM), lysine mimetic, and hydrogen.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith. M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle.” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. In some embodiments, a carbocyclic ring may be a 5-12 membered bicyclic, bridged bicyclic, or spirocyclic ring. A carbocyclic ring may include one or more oxo (═O) or thioxo (═S) substituent. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, and sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Without limitation, a bridged bicyclic group may contain two or more bridges, e.g., adamantanyl. Exemplary bridged bicyclics include:

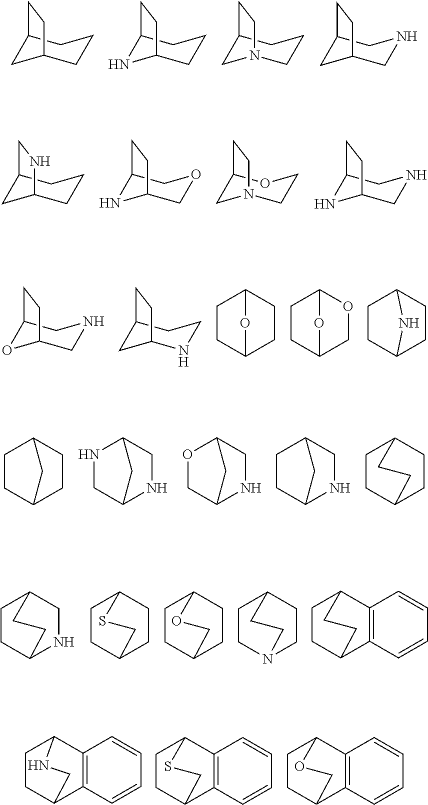

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

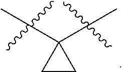 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms: having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring.” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl.” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H pyrrolyl), NH (as in pyrrolidinyl), or ⁺NR (as in N substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring.” “heterocyclic group,” “heterocyclic moiety,” and “heterocyclic radical.” are used interchangeably herein, and also include groups in which a heterocyclyl ring is fused to one or more aryl, heteroaryl, or cycloaliphatic rings, such as indolinyl, 3H indolyl, chromanyl, phenanthridinyl, or tetrahydroquinolinyl. In some embodiments, a heterocyclic ring may be a 5-12 membered bicyclic, bridged bicyclic, or spirocyclic ring. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the disclosure may contain “substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen: —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R°; —N(R^{∘})C(S)R^{∘}; —(CH₂)₀₋₄N(R^{∘})C(O)NR^{∘}₂; —N(R^{∘}C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)OR^{∘}; —(CH₂)₀₋₄OS(O)R^{∘}; —S(O)₂NR^{∘}₂; —(CH₂)₀₋₄S(O)R^{∘}; —N(R^{∘})S(O)NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, and sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromotagraphic purification are comtemplated herein (e.g., diammonium salts) and are readily apparent to those having skill in the art.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “provided compound” refers to any genus, subgenus, and/or species set forth herein.

The term “prodrug” refers to a compound that is made more active in vivo. The present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

As used herein, the term “inhibitor” is defined as a compound that binds to and/or inhibits BCL-XL and BCL-2 protein with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional compound that binds to and/or inhibits BCL-XL and BCL-2 protein and an E3 ligase with measurable affinity resulting in the ubiquitination and subsequent degradation of the BCL-XL and BCL-2 protein. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a degrader compound without an appended E3 ligase binding moiety.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41:2596-99 and Sun et al., Bioconjugate Chem., 2006, 17:52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE. Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4-[(p-Methoxytetrafluorobenzyl)oxy]phenyl]-3-methylglyceronyl]isonipecotic Acid, 4-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in BCL-XL and BCL-2 protein activity between a sample comprising a compound of the present invention, or composition thereof, and BCL-XL and BCL-2 protein, and an equivalent sample comprising BCL-XL and BCL-2 protein, in the absence of said compound, or composition thereof.

### 3. Description of Exemplary Embodiments

As described above, in certain embodiments, the present invention provides a compound of formula I:

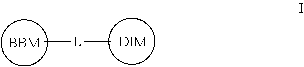

or a pharmaceutically acceptable salt thereof, wherein:
BBM is a BCL-XL and BCL-2 protein binding moiety capable of binding to BCL-XL and BCL-2;
L is a bivalent moiety that connects BBM to DIM; and
DIM is a degradation inducing moiety selected from an E3 ubiquitin ligase binding moeity (LBM), lysine mimetic, and hydrogen.

### BCL-XL Binding Moiety (BBM)

As described and defined herein, BBM is a BCL-XL and BCL-2 protein binding moiety. In some embodiments, BBM is a selective BCL-XL and BCL-2 protein binding moiety. In some embodiments, BBM binds selectively to BCL-XL and BCL-2 over other anti-apoptotic BCL-2 family proteins, such as BCL-W and MCL-1. In some embodiments, the present invention provide compounds that are dual BCL-XL and BCL-2 degraders.

Such binders are well known to one of ordinary skill in the art and including ABT-737 (US 20070072860), navitoclax (ABT-263, WO 2009155386), venetoclax (ABT-199, WO 2010138588), obatoclax (GX 15-070, WO 2004106328), pelcitoclax (APG-1252), (−)-gossypol (AT-101, WO 2002097053), sabutoclax (BI-97C1, WO 2010120943), TW-37 (WO 2006023778), BM-1252 (APG-1252), A-1155463 (WO 2010080503 and WO 2010080478), A-1293102, A-1331852 (WO 2013055897 and WO 2013055895), AZD4320 (WO 2012017251), WEHI-539 (Lessene, Guillaume, et al. “Structure-guided design of a selective BCL-XL inhibitor.” Nature Chem. Bio. 2013, 9 (6):390-397), and other binders disclosed by the University of Michighan (WO 2018027097), Genentech (WO 2008061208), Novartis (WO2011029842A1), and Servier (WO 2021018857 and WO 2021018858; S44563), the entirety of each or which is herein incorporated by reference.

As defined herein and described below, wherein a formula is depicted using square brackets, e.g,

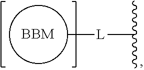

L is attached to a modifiable carbon, oxygen, or nitrogen atom within BBM including substitution or replacement of a defined group in BBM.

In certain embodiments, the present invention provides a compound of formula I, wherein BBM is a BCL-XL and BCL-2 binding moiety thereby forming a compound of formula I-aa:

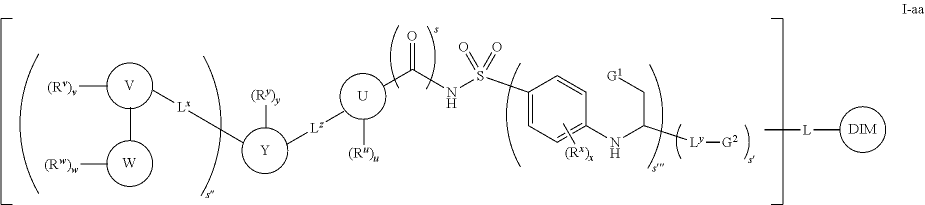

or a pharmaceutically acceptable salt, wherein L and DIM are as defined above and described in embodiments herein, and wherein:
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Ring W is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 4-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
G¹ is —S-aryl, —S-heteroaryl, or —R^{A};
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
G² is hydrogen, halogen, —CN, —OR, —SR, —N(R)₂, —S(O)₂R, —S(O)₂N(R)₂, —C(O)R, —C(O)OR, or

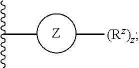

each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
Ring Z is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
R^{u}, R^{v}, R^{w}, R^{x}, R^{y}, and R^{z} are, independently, hydrogen, halogen, C₁₋₆alkyl, C₁₋₆haloalkyl, —CN, —NO₂, —OR, —SR, —N(R)₂, —Si(R)₃, —S(O)₂R, —S(O)₂N(R)₂, —S(O)₂NRC(O)R—S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, —NRS(O)₂R, or R^{A};
L^{x}, L^{y}, and L^{z} are, independently, a covalent bond or a bivalent, saturated or partially unsaturated, straight or branched C₁₋₅ hydrocarbon chain, wherein 0-3 methylene units of L^{x}, L^{y}, and L^{z} are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
S, s″, and s′″ are, independently, 0 or 1;
s′ is 1 or 2; and
u, v, w, x, y, and z are, independently, 0, 1, 2, 3, or 4.

In certain embodiments, the present invention provides a compound of formula I, wherein BBM is a BCL-XL and BCL-2 binding moiety thereby forming a compound of formula I-bb:

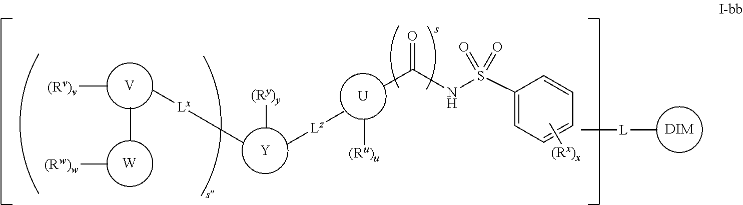

or a pharmaceutically acceptable salt, wherein L and DIM are as defined above and described in embodiments herein, and wherein:
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Ring W is a ring selected from phenyl, naphthyl, a 5-10 membered heteroaryl containing 1-4 heteroatoms independently selected from nitrogen, oxygen, selenium, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 4-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{u}, R^{v}, R^{w}, R^{x}, and R^{y} are, independently, hydrogen, halogen, C₁₋₆alkyl, C₁₋₆haloalkyl, —CN, —NO₂, —OR, —SR, —N(R)₂, —Si(R)₃, —S(O)₂R, —S(O)₂N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, —NRS(O)₂R, or R^{A};
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or;
two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} and L^{z} are, independently, a covalent bond or a bivalent, saturated or partially unsaturated, straight or branched C₁₋₅ hydrocarbon chain, wherein 0-3 methylene units of L^{x} and L^{z} are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
s and s″ are, independently, 0 or 1; and
u, v, w, x, and y are, independently, 0, 1, 2, 3, or 4.

In certain embodiments, the present invention provides a compound of formula I, wherein BBM is a BCL-XL and BCL-2 binding moiety thereby forming a compound of formula I-cc:

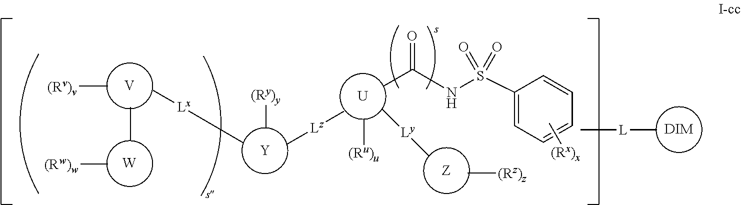

or a pharmaceutically acceptable salt, wherein L and DIM are as defined above and described in embodiments herein, and wherein:
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Ring W and Ring Z are, independently, a ring selected from phenyl, naphthyl, a 5-10 membered heteroaryl containing 1-4 heteroatoms independently selected from nitrogen, oxygen, selenium, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 4-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{u}, R^{v}, R^{w}, R^{x}, R^{y}, and R^{z} are, independently, hydrogen, halogen, C₁₋₆alkyl, C₁₋₆haloalkyl, —CN, —NO₂, —OR, —SR, —N(R)₂, —Si(R)₃, —S(O)₂R, —S(O)₂N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, —NRS(O)₂R, or R^{A};
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x}, L^{y}, and L^{z} are, independently, a covalent bond or a bivalent, saturated or partially unsaturated, straight or branched C₁₋₅ hydrocarbon chain, wherein 0-3 methylene units of L^{x}, L^{y}, and L^{z} are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
s and s″ are, independently, 0 or 1; and
u, v, w, x, y, and z are, independently, 0, 1, 2, 3, or 4.

As defined above and described herein, Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur.

In some embodiments, Ring U is phenylenyl. In some embodiments, Ring U is a 5-15 membered saturated or partially unsaturated heterocyclylenyl with 1-4 heteroatoms independently selected from nitrogen, oxygen, and sulfur. In some embodiments, Ring U is a 5-10 membered heteroarylenyl containing 1-4 heteroatoms independently selected from nitrogen, oxygen, and sulfur.

In some embodiments, Ring U is

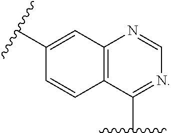 [SMILES: *c1ccc2c(*)ncnc2c1]

In some embodiments, Ring U is

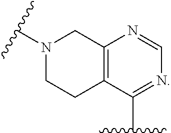 [SMILES: *c1ncnc2c1CCN(*)C2]

In some embodiments, Ring U is

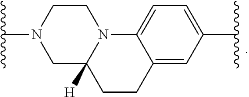 [SMILES: *c1ccc2c(c1)CC[C@@H]1CN(*)CCN21]

In some embodiments, Ring U is selected from those depicted in Table 1, below.

As defined above and described herein, Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur.

In some embodiments, Ring V is a 5-6 membered saturated or partially unsaturated carbocyclylenyl. In some embodiments, Ring V is a 5-6 membered saturated or partially unsaturated heterocyclylenyl with 1-4 heteroatoms independently selected from nitrogen, oxygen, and sulfur. In some embodiments, Ring V is is a 5-6 membered heterocyclylenyl with 1-4 heteroatoms independently selected from nitrogen, oxygen, and sulfur. In some embodiments, Ring V is a 5-6 membered heteroarylenyl with 1-4 heteroatoms independently selected from nitrogen, oxygen, and sulfur.

In some embodiments, Ring V is cyclohexenyl. In some embodiments, Ring V is pyrrolylenyl.

In some embodiments, Ring V is selected from those depicted in Table 1, below.

As defined above and described herein, Ring W and Ring Z are, independently, a ring selected from phenyl, naphthyl, a 5-10 membered heteroaryl containing 1-4 heteroatoms independently selected from nitrogen, oxygen, selenium, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur.

In some embodiments, Ring W is phenyl. In some embodiments, Ring W is naphthyl. In some embodiments, Ring W is a 5-10 membered heteroaryl containing 1-4 heteroatoms independently selected from nitrogen, oxygen, selenium, and sulfur. In some embodiments, Ring W is a 5-11 membered saturated or partially unsaturated monocyclic, bicyclic, bridged bicylic, or spirocyclic carbocyclyl. In some embodiments, Ring W is a 5-11 membered saturated or partially unsaturated monocyclic, bicyclic, bridged bicylic, or spirocyclic heterocyclyl with 1-4 heteroatoms independently selected from nitrogen, oxygen, and sulfur.

In some embodiments, Ring W is benzothiazolyl. In some embodiments, Ring W is

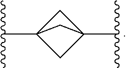 [SMILES: *C12CC(*)(C1)C2]

In some embodiments, Ring Z is phenyl. In some embodiments, Ring Z is naphthyl. In some embodiments, Ring Z is a 5-10 membered heteroaryl containing 1-4 heteroatoms independently selected from nitrogen, oxygen, selenium, and sulfur. In some embodiments, Ring Z is a 5-11 membered saturated or partially unsaturated monocyclic, bicyclic, bridged bicylic, or spirocyclic carbocyclyl. In some embodiments, Ring Z is a 5-11 membered saturated or partially unsaturated monocyclic, bicyclic, bridged bicylic, or spirocyclic heterocyclyl with 1-4 heteroatoms independently selected from nitrogen, oxygen, and sulfur.

In some embodiments, Ring Z is cyclohexyl. In some embodiments, Ring Z is piperzinyl. In some embodiments, Ring Z is morpholinyl. In some embodiments, Ring Z is pyridyl. In some embodiments, Ring Z is pyrazolyl.

In some embodiments, Ring Z is

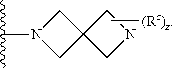

In some embodiments, Ring Z is

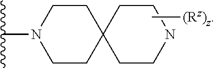

In some embodiments, Ring Z is

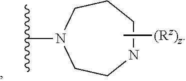

In some embodiments, Ring Z is

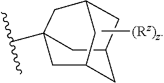

In some embodiments, Ring Z is

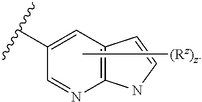

In some embodiments, Ring Z is

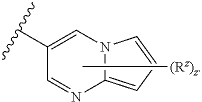

In some embodiments, Ring Z is

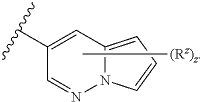

In some embodiments, Ring W and Ring Z are selected from those depicted in Table 1, below.

As defined above and described herein, Ring Y is a bivalent ring selected from phenyl or phenylenyl, a 4-11 membered saturated or partially unsaturated monocyclic, bicyclic, bridged bicyclic, or spirocyclic carbocyclyl or carbocyclylenyl or heterocyclyl or heterocyclylenyl with 1-4 heteroatoms independently selected from nitrogen, oxygen, and sulfur, and a 5-6 membered heteroaryl or heteroarylenyl containing 1-4 heteroatoms independently selected from nitrogen, oxygen, and sulfur.

In some embodiments, Ring Y is phenylenyl. In some embodiments, Ring Y is a 4-11 membered saturated or partially unsaturated monocyclic, bicyclic, bridged bicyclic, or spirocyclic carbocyclylenyl. In some embodiments, Ring Y is a 4-11 membered saturated or partially unsaturated monocyclic, bicyclic, bridged bicyclic, or spirocyclic heterocyclylenyl with 1-4 heteroatoms independently selected from nitrogen, oxygen, and sulfur. In some embodiments, Ring Y is a 5-6 membered heteroarylenyl containing 1-4 heteroatoms independently selected from nitrogen, oxygen, and sulfur.

In some embodiments, Ring Y is phenyl. In some embodiments, Ring Y is a 4-11 membered saturated or partially unsaturated monocyclic, bicyclic, bridged bicyclic, or spirocyclic carbocyclyl. In some embodiments, Ring Y is a 4-11 membered saturated or partially unsaturated monocyclic, bicyclic, bridged bicyclic, or spirocyclic heterocyclyl with 1-4 heteroatoms independently selected from nitrogen, oxygen, and sulfur. In some embodiments, Ring Y is a 5-6 membered heteroaryl containing 1-4 heteroatoms independently selected from nitrogen, oxygen, and sulfur.

In some embodiments, Ring Y is pyridylenyl. In some embodiments, Ring Y is thiazoylenyl. In some embodiments, Ring Y is piperzinylenyl. In some embodiments, Ring Y is

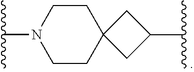 [SMILES: *C1CC2(CCN(*)CC2)C1]

In some embodiments, Ring W is a

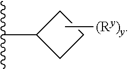

In some embodiments, Ring W is a

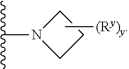

In some embodiments, Ring Y is

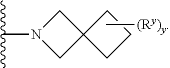

In some embodiments, Ring Y is

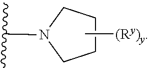

In some embodiments, Ring Y is

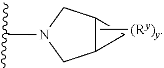

In some embodiments, Ring Y is

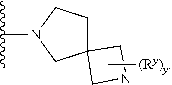

In some embodiments, Ring Y is

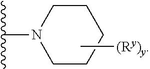

In some embodiments, Ring Y is

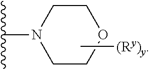

In some embodiments, Ring Y is

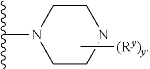

In some embodiments, Ring Y is

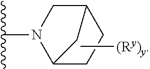

In some embodiments, Ring Y is

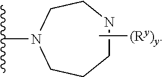

In some embodiments, Ring Y and its R^{y} substitutent is

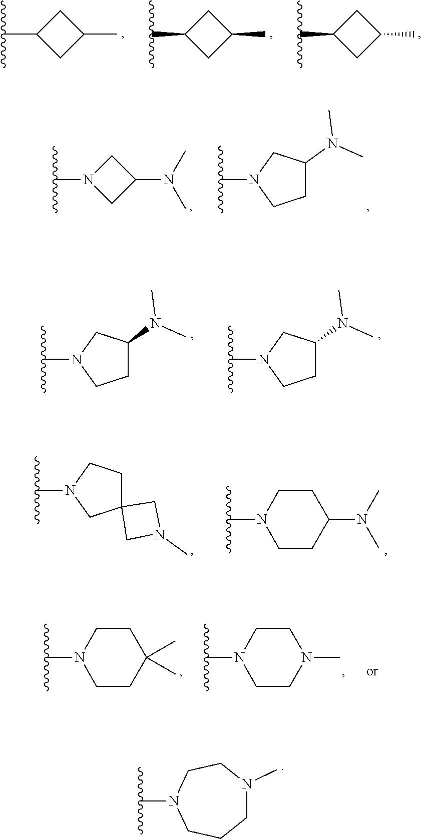

In some embodiments, Ring Y is selected from those depicted in Table 1, below.

As defined above and described herein, G¹ is —S-aryl, —S-heteroaryl, or —R^{A}.

In some embodiments, G¹ is —S-aryl. In some embodiments, G¹ is —S-heteroaryl. In some embodiments, G¹ is —R^{A}.

In some embodiments, G¹ is —SPh.

In some embodiments, G¹ is selected from those depicted in Table 1, below.

As defined above and described herein, G² is hydrogen, R^{A}, halogen, —CN, —OR, —SR, —N(R)₂, —S(O)₂R, —S(O)₂N(R)₂, —C(O)R, —C(O)OR, or

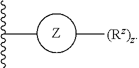

In some embodiments, G² is hydrogen. In some embodiments, G² is R^{A}. In some embodiments, G² is halogen. In some embodiments, G² is —CN. In some embodiments, G² is —OR. In some embodiments, G² is —SR. In some embodiments, G² is —N(R)₂. In some embodiments, G² is —S(O)₂R. In some embodiments, G² is —S(O)₂N(R)₂. In some embodiments, G² is —C(O)R. In some embodiments, G² is —C(O)OR. In some embodiments, G² is

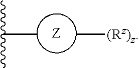

In some embodiments, G² is —OH. In some embodiments, G² is —NMe₂.

In some embodiments, G² is selected from those depicted in Table 1, below.

As defined above and described herein, R^{u}, R^{v}, R^{w}, R^{x}, R^{y}, and R^{z} are, independently, hydrogen, halogen, C₁₋₆alkyl, C₁₋₆haloalkyl, —CN, —NO₂, —OR, —SR, —N(R)₂, —Si(R)₃, —S(O)₂R, —S(O)₂N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, —NRS(O)₂R, or R^{A};

In some embodiments, one or more of R^{u}, R^{v}, R^{w}, R^{x}, R^{y}, and R^{z} are hydrogen. In some embodiments, one or more of R^{u}, R^{v}, R^{w}, R^{x}, R^{y}, and R^{z} are C₁₋₆alkyl. In some embodiments, one or more of R^{u}, R^{v}, R^{w}, R^{x}, R^{y}, and R^{z} are C₁₋₆haloalkyl (e.g., —CF₃, —CHF₂, etc.). In some embodiments, one or more of R^{u}, R^{v}, R^{w}, R^{x}, R^{y}, and R^{z} are halogen. In some embodiments, one or more of R^{u}, R^{v}, R^{w}, R^{x}, R^{y}, and R^{z} are —CN. In some embodiments, one or more of R^{u}, R^{v}, R^{w}, R^{x}, R^{y}, and R^{z} are —NO₂. In some embodiments, one or more of R^{u}, R^{v}, R^{w}, R^{x}, R^{y}, and R^{z} are —OR. In some embodiments, one or more of R^{u}, R^{v}, R^{w}, R^{x}, R^{y}, and R^{z} are —SR. In some embodiments, one or more of R^{u}, R^{v}, R^{w}, R^{x}, R^{y}, and R^{z} are —N(R)₂. In some embodiments, one or more of R^{u}, R^{v}, R^{w}, R^{x}, R^{y}, and R^{z} are —Si(R)₂. In some embodiments, one or more of R^{u}, R^{v}, R^{w}, R^{x}, R^{y}, and R^{z} are —S(O)₂R. In some embodiments, one or more of R^{u}, R^{v}, R^{w}, R^{x}, R^{y}, and R^{z} are —S(O)₂N(R)₂. In some embodiments, one or more of R^{u}, R^{v}, R^{w}, R^{x}, R^{y}, and R^{z} are —S(O)₂NRC(O)R. In some embodiments, one or more of R^{u}, R^{v}, R^{w}, R^{x}, R^{y}, and R^{z} are —S(O)R. In some embodiments, one or more of R^{u}, R^{v}, R^{w}, R^{x}, R^{y}, and R^{z} are —S(O)₂OR. In some embodiments, one or more of R^{u}, R^{v}, R^{w}, R^{x}, R^{y}, and R^{z} are —C(O)R. In some embodiments, one or more of R^{u}, R^{v}, R^{w}, R^{x}, R^{y}, and R^{z} are —C(O)OR. In some embodiments, one or more of R^{u}, R^{v}, R^{w}, R^{x}, R^{y}, and R^{z} are —C(O)N(R)₂. In some embodiments, one or more of R^{u}, R^{v}, R^{w}, R^{x}, R^{y}, and R^{z} are —C(O)NROR. In some embodiments, one or more of R^{u}, R^{v}, R^{w}, R^{x}, R^{y}, and R^{z} are —C(O)NRC(O)R. In some embodiments, one or more of R^{u}, R^{v}, R^{w}, R^{x}, R^{y}, and R^{z} are —C(O)NRS(O)₂R. In some embodiments, one or more of R^{u}, R^{v}, R^{w}, R^{x}, R^{y}, and R^{z} are —OC(O)R. In some embodiments, one or more of R^{u}, R^{v}, R^{w}, R^{x}, R^{y}, and R^{z} are —OC(O)N(R)₂. In some embodiments, one or more of R^{u}, R^{v}, R^{w}, R^{x}, R^{y}, and R^{z} are —OP(O)(R)₂. In some embodiments, one or more of R^{u}, R^{v}, R^{w}, R^{x}, R^{y}, and R^{z} are —OP(O)(OR)₂. In some embodiments, one or more of R^{u}, R^{v}, R^{w}, R^{x}, R^{y}, and R^{z} are —OP(O)(OR)N(R)₂. In some embodiments, one or more of R^{u}, R^{v}, R^{w}, R^{x}, R^{y}, and R^{z} are —OP(O)(N(R)₂)₂. In some embodiments, one or more of R^{u}, R^{v}, R^{w}, R^{x}, R^{y}, and R^{z} are —NRC(O)OR. In some embodiments, one or more of R^{u}, R^{v}, R^{w}, R^{x}, R^{y}, and R^{z} are —NRC(O)R. In some embodiments, one or more of R^{u}, R^{v}, R^{w}, R^{x}, R^{y}, and R^{z} are —NRC(O)N(R)₂. In some embodiments, one or more of R^{u}, R^{v}, R^{w}, R^{x}, R^{y}, and R^{z} are —NRS(O)₂R. In some embodiments, one or more of R^{u}, R^{v}, R^{w}, R^{x}, R^{y}, and R^{z} are —NP(O)(R)₂. In some embodiments, one or more of R^{u}, R^{v}, R^{w}, R^{x}, R^{y}, and R^{z} are —NRP(O)(OR)₂. In some embodiments, one or more of R^{u}, R^{v}, R^{w}, R^{x}, R^{y}, and R^{z} are —NRP(O)(OR)N(R)₂. In some embodiments, one or more of R^{u}, R^{v}, R^{w}, R^{x}, R^{y}, and R^{z} are —NRP(O)(N(R)₂)₂. In some embodiments, one or more of R^{u}, R^{v}, R^{w}, R^{x}, R^{y}, and R^{z} are —NRS(O)₂R. In some embodiments, one or more of R^{u}, R^{v}, R^{w}, R^{x}, R^{y}, and R^{z} are R^{A}.

In some embodiments, R^{v} is C₁₋₆alkyl. In some embodiments, R^{v} is methyl. In some embodiments, R^{v} is —NO₂. In some embodiments, R^{v} is —S(O)₂CF₃.

In some embodiments, R^{w} is halogen. In some embodiments, R^{w} is chloro. In some embodiments, R^{v} is C₁₋₆alkyl. In some embodiments, R^{v} is cyclopropyl. In some embodiments, R^{w} is C₁₋₆haloalkyl. In some embodiments, R^{w} is —CF₂H.

In some embodiments, R^{x} is —NO₂. In some embodiments, R^{x} is —S(O)₂CF₃. In some embodiments, R^{x} is —NR₂. In some embodiments, R^{x} is —NH₂. In some embodiments, R^{x} is

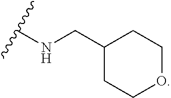 [SMILES: *NCC1CCOCC1]

In some embodiments, R^{x} is

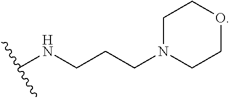 [SMILES: *NCCCN1CCOCC1]

In some embodiments, Ring R^{x} is

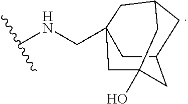 [SMILES: *NCC12CC3CC(CC(O)(C3)C1)C2]

In some embodiments, R^{y} is —CO₂H. In some embodiments, R^{y} is a carboxylic acid isostere known in the art, e.g., Ballatore et al., “Carboxylic acid (bio) isosteres in drug design.” ChemMedChem 2012, 8 (3):385.

In some embodiments, R^{z} is halogen. In some embodiments, R^{z} is fluoro. In some embodiments, R^{z} is —C(O)R. In some embodiments, R^{z} is acetyl. In some embodiments, R^{z} is C₁₋₆alkyl. In some embodiments, R^{z} is methyl. In some embodiments, R^{z} is C₁₋₄haloalkyl. In some embodiments, R^{z} is —CF₂H. In some embodiments, R^{x} is —NR₂. In some embodiments, R^{z} is

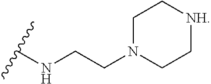 [SMILES: *NCCN1CCNCC1]

In some embodiments, R^{z} is —OR. In some embodiments, R^{z} is —OH. In some embodiments, R^{z} is R^{A}. In some embodiments, R^{z} is —CH₂NH₂. In some embodiments, R^{z} is

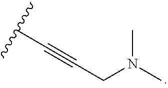 [SMILES: *C#CCN(C)C]

In some embodiments, R^{z} —(CH₂)₂CO₂H. In some embodiments, R^{z} is

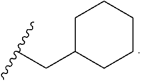 [SMILES: *CC1CCCCC1]

In some embodiments, R^{z} is

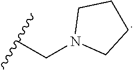 [SMILES: *CN1CCCC1]

In some embodiments, R^{z} is

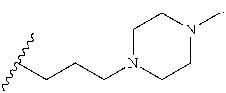 [SMILES: *CCCN1CCN(C)CC1]

In some embodiments, R^{z} is

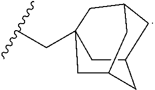 [SMILES: *CC12CC3CC(CC(C3)C1)C2]

In some embodiments, Ring R^{z} is

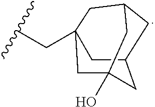 [SMILES: *CC12CC3CC(CC(O)(C3)C1)C2]

In some embodiments, R^{u}, R^{v}, R^{w}, R^{x}, R^{y}, and R^{z} are selected from those depicted in Table 1, below.

As defined above and described herein, each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur.

In some embodiments, R is hydrogen. In some embodiments, R is optionally substituted C₁₋₆ aliphatic. In some embodiments, R is optionally substituted phenyl. In some embodiments, R is optionally substituted 3-7 membered saturated or partially unsaturated heterocyclic ring having 1-2 heteroatoms independently selected from nitrogen, oxygen, and sulfur. In some embodiments, R is optionally substituted 5-6 membered heteroaryl ring having 1-4 heteroatoms independently selected from nitrogen, oxygen, and sulfur. In some embodiments, two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur.

In some embodiments, Ring R is selected from those depicted in Table 1, below.

As defined above and described herein, each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is independently an optionally substituted C₁₋₆ aliphatic. In some embodiments, R^{A} is independently an optionally substituted phenyl. In some embodiments, R^{A} is independently an optionally substituted 3-10 membered saturated or partially unsaturated carbocyclic ring. In some embodiments, R^{A} is independently an optionally substituted 3-7 membered saturated or partially unsaturated heterocyclic ring having 1-2 heteroatoms independently selected from nitrogen, oxygen, and sulfur. In some embodiments, R^{A} is independently an optionally substituted 5-6 membered heteroaryl ring having 1-4 heteroatoms independently selected from nitrogen, oxygen, and sulfur.

In some embodiments, R^{A} is

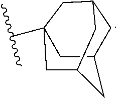 [SMILES: *C12CC3CC(CC(C3)C1)C2]

In some embodiments, Ring R^{A} is

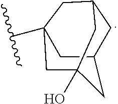 [SMILES: *C12CC3CC(C1)CC(O)(C3)C2]

In some embodiments, R^{A} is selected from those depicted in Table 1, below.

As defined above and described herein, L^{x}, L^{y}, and L^{z} are, independently, a covalent bond or a bivalent, saturated or partially unsaturated, straight or branched C₁₋₅ hydrocarbon chain, wherein 0-3 methylene units of L¹ are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—.

In some embodiments, L^{x} is a covalent bond. In some embodiments, L^{y} is a covalent bond. In some embodiments, L^{z} is a covalent bond. In some embodiments, L^{x} is a bivalent, saturated or partially unsaturated, straight or branched C₁₋₅ hydrocarbon chain, wherein 0-3 methylene units of L^{x} are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—. In some embodiments, L^{y} is a bivalent, saturated or partially unsaturated, straight or branched C₁₋₅ hydrocarbon chain, wherein 0-3 methylene units of L^{y} are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—. In some embodiments, L^{z} is a bivalent, saturated or partially unsaturated, straight or branched C₁₋₅ hydrocarbon chain, wherein 0-3 methylene units of L^{y} are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—.

In some embodiments, L^{x} is —CH₂—. In some embodiments, L^{x} is

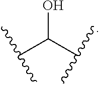 [SMILES: *C(*)O]

In some embodiments, L^{x} is

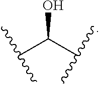

In some embodiments, L^{x} is

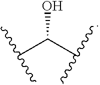

In some embodiments, L^{x} is

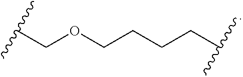 [SMILES: *CCCCOC*]

In some embodiments, L^{x} is

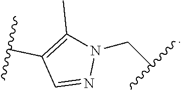 [SMILES: *Cn1ncc(*)c1C]

In some embodiments, L^{y} is —CH₂—. In some embodiments, L^{y} is

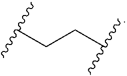 [SMILES: *CC*]

In some embodiments, L^{y} is

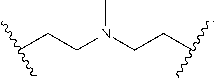 [SMILES: *CCN(C)CC*]

In some embodiments, L^{z} is —CH₂—. In some embodiments, L^{z} is —O—. In some embodiments, L^{z} is —NR—. In some embodiments, L^{z} is —NH—.

In some embodiments, L^{x}, L^{y}, and L^{z} are selected from those depicted in Table 1, below.

As defined above and described herein, X^{a} and X^{b} are, independently, a carbon atom or a nitrogen atom.

In some embodiments, X^{a} is a carbon atom. In some embodiments, X^{a} is a nitrogen atom. In some embodiments, X^{b} is a carbon atom. In some embodiments, X^{b} is a nitrogen atom.

In some embodiments, X^{a} and X^{b} are selected from those depicted in Table 1, below.

As defined above and described herein, s, s′, and s″ are, independently, 0, 1, or 2.

In some embodiments, s is 0. In some embodiments, s′ is 0. In some embodiments, s″ is 0.

In some embodiments, s is 1. In some embodiments, s′ is 1. In some embodiments, s″ is 1. In some embodiments, s is 2. In some embodiments, s′ is 2. In some embodiments, s″ is 2.

In some embodiments, s and s′ are selected from those depicted in Table 1, below.

As defined above and described herein, u, v, w, x, y, and z are, independently, 0, 1, 2, 3, or 4.

In some embodiments, u is 0. In some embodiments, u is 1. In some embodiments, u is 2. In some embodiments, u is 3. In some embodiments, u is 4. In some embodiments, v is 0. In some embodiments, v is 1. In some embodiments, v is 2. In some embodiments, v is 3. In some embodiments, v is 4. In some embodiments, w is 0. In some embodiments, w is 1. In some embodiments, w is 2. In some embodiments, w is 3. In some embodiments, w is 4. some embodiments, x is 0. In some embodiments, x is 1. In some embodiments, x is 2. In some embodiments, x is 3. In some embodiments, x is 4. In some embodiments, y is 0. In some embodiments, y is 1. In some embodiments, y is 2. In some embodiments, y is 3. In some embodiments, y is 4. In some embodiments, z is 0. In some embodiments, z is 1. In some embodiments, z is 2. In some embodiments, z is 3. In some embodiments, z is 4.

In some embodiments, u, v, w, y, and z are selected from those depicted in Table 1, below.

In some embodiments, BBM is

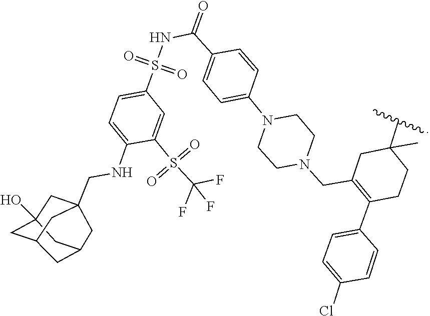 [SMILES: *C1(C)CCC(c2ccc(Cl)cc2)=C(CN2CCN(c3ccc(C(=O)NS(=O)(=O)c4ccc(NCC56CC7CC(CC(O)(C7)C5)C6)c(S(=O)(=O)C(F)(F)F)c4)cc3)CC2)C1]

In some embodiments, BBM is

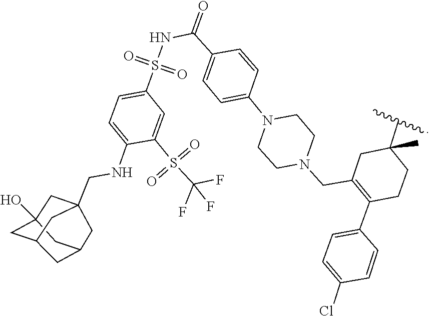 [SMILES: *[C@]1(C)CCC(c2ccc(Cl)cc2)=C(CN2CCN(c3ccc(C(=O)NS(=O)(=O)c4ccc(NCC56CC7CC(CC(O)(C7)C5)C6)c(S(=O)(=O)C(F)(F)F)c4)cc3)CC2)C1]

In some embodiments, BBM is

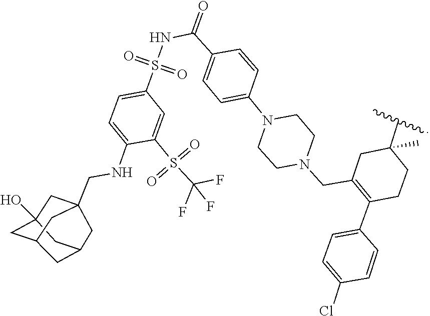 [SMILES: *[C@@]1(C)CCC(c2ccc(Cl)cc2)=C(CN2CCN(c3ccc(C(=O)NS(=O)(=O)c4ccc(NCC56CC7CC(CC(O)(C7)C5)C6)c(S(=O)(=O)C(F)(F)F)c4)cc3)CC2)C1]

In some embodiments, BBM is

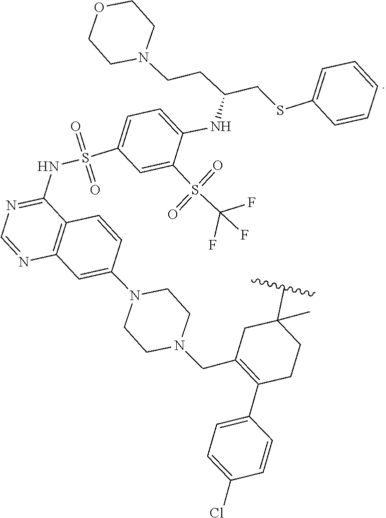 [SMILES: *C1(C)CCC(c2ccc(Cl)cc2)=C(CN2CCN(c3ccc4c(NS(=O)(=O)c5ccc(N[C@H](CCN6CCOCC6)CSc6ccccc6)c(S(=O)(=O)C(F)(F)F)c5)ncnc4c3)CC2)C1]

In some embodiments, BBM is

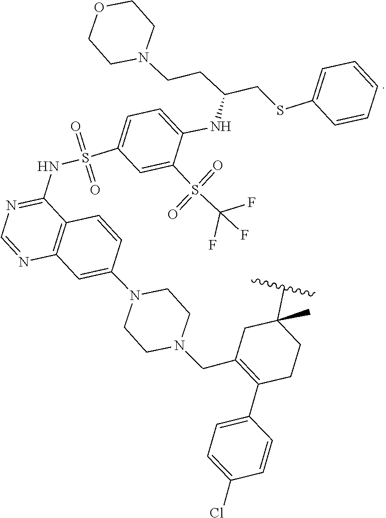 [SMILES: *[C@]1(C)CCC(c2ccc(Cl)cc2)=C(CN2CCN(c3ccc4c(NS(=O)(=O)c5ccc(N[C@H](CCN6CCOCC6)CSc6ccccc6)c(S(=O)(=O)C(F)(F)F)c5)ncnc4c3)CC2)C1]

In some embodiments, BBM is

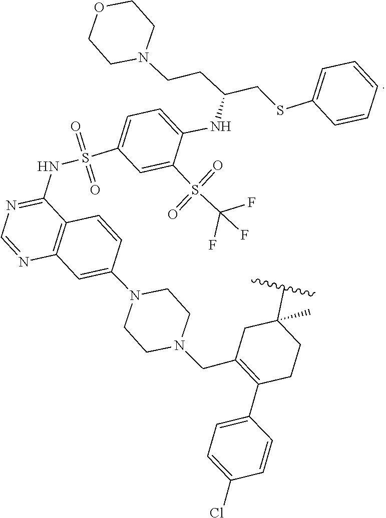 [SMILES: *[C@@]1(C)CCC(c2ccc(Cl)cc2)=C(CN2CCN(c3ccc4c(NS(=O)(=O)c5ccc(N[C@H](CCN6CCOCC6)CSc6ccccc6)c(S(=O)(=O)C(F)(F)F)c5)ncnc4c3)CC2)C1]

In some embodiments, BBM is

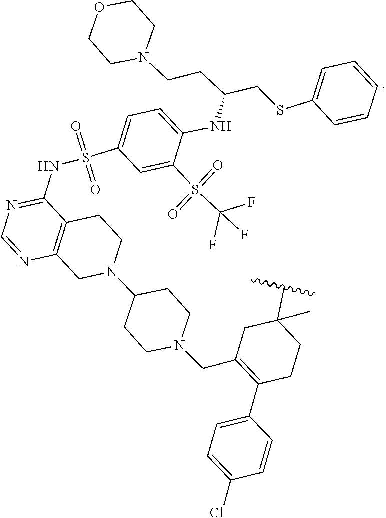 [SMILES: *C1(C)CCC(c2ccc(Cl)cc2)=C(CN2CCC(N3CCc4c(ncnc4NS(=O)(=O)c4ccc(N[C@H](CCN5CCOCC5)CSc5ccccc5)c(S(=O)(=O)C(F)(F)F)c4)C3)CC2)C1]

In some embodiments, BBM is

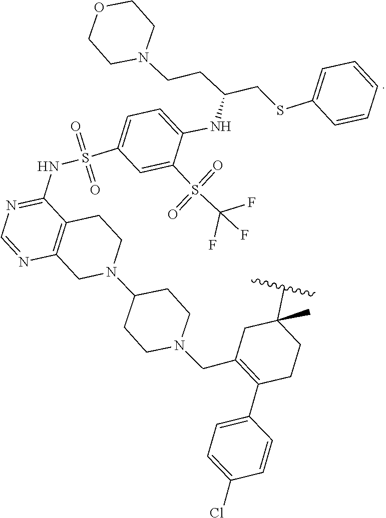 [SMILES: *[C@]1(C)CCC(c2ccc(Cl)cc2)=C(CN2CCC(N3CCc4c(ncnc4NS(=O)(=O)c4ccc(N[C@H](CCN5CCOCC5)CSc5ccccc5)c(S(=O)(=O)C(F)(F)F)c4)C3)CC2)C1]

In some embodiments, BBM is

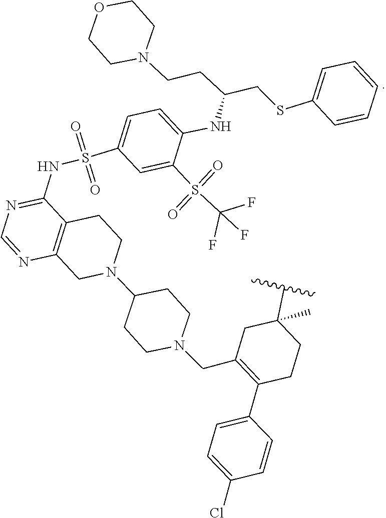 [SMILES: *[C@@]1(C)CCC(c2ccc(Cl)cc2)=C(CN2CCC(N3CCc4c(ncnc4NS(=O)(=O)c4ccc(N[C@H](CCN5CCOCC5)CSc5ccccc5)c(S(=O)(=O)C(F)(F)F)c4)C3)CC2)C1]

In some embodiments, BBM is

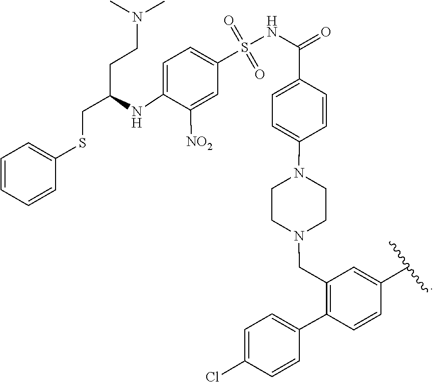 [SMILES: *c1ccc(-c2ccc(Cl)cc2)c(CN2CCN(c3ccc(C(=O)NS(=O)(=O)c4ccc(N[C@H](CCN(C)C)CSc5ccccc5)c([N+](=O)[O-])c4)cc3)CC2)c1]

In some embodiments, BBM is

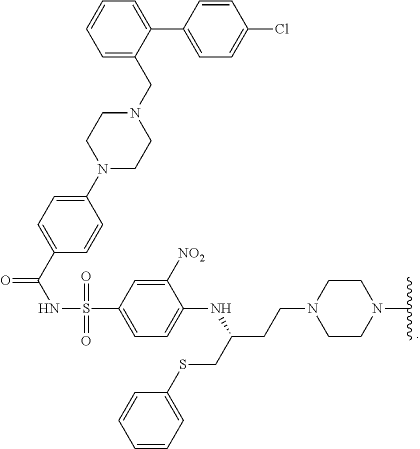 [SMILES: *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1]

In some embodiments, BBM is

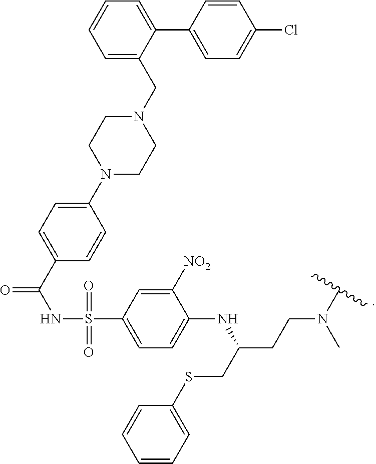 [SMILES: *N(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]

In some embodiments, BBM is

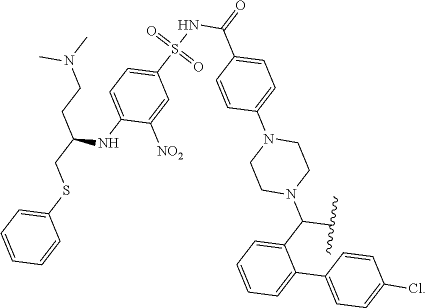 [SMILES: *C(c1ccccc1-c1ccc(Cl)cc1)N1CCN(c2ccc(C(=O)NS(=O)(=O)c3ccc(N[C@H](CCN(C)C)CSc4ccccc4)c([N+](=O)[O-])c3)cc2)CC1]

In some embodiments, BBM is is

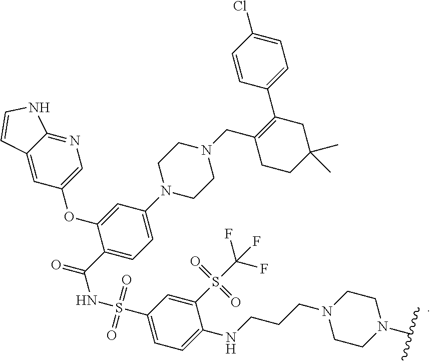 [SMILES: *N1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)CC1]

In some embodiments, BBM is

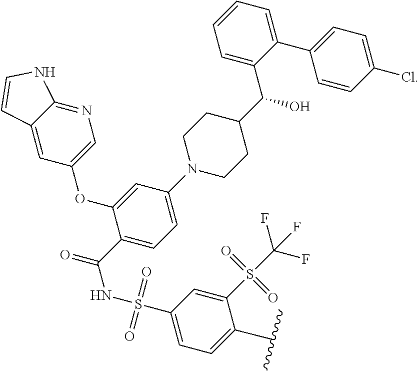 [SMILES: *c1ccc(S(=O)(=O)NC(=O)c2ccc(N3CCC([C@@H](O)c4ccccc4-c4ccc(Cl)cc4)CC3)cc2Oc2cnc3[nH]ccc3c2)cc1S(=O)(=O)C(F)(F)F]

In some embodiments, BBM is

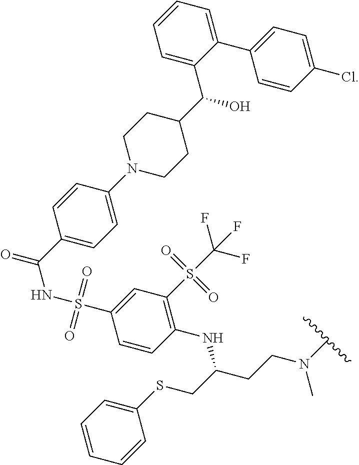 [SMILES: *N(C)CC[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F]

In some embodiments, BBM is

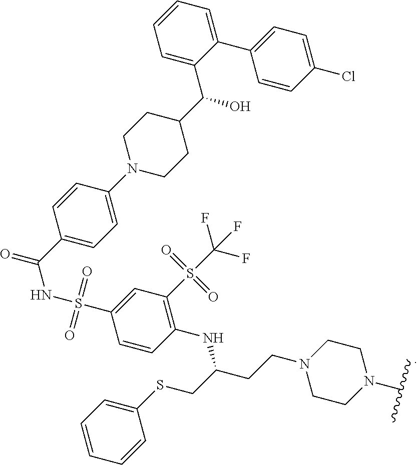 [SMILES: *N1CCN(CC[C@H](CSc2ccccc2)Nc2ccc(S(=O)(=O)NC(=O)c3ccc(N4CCC([C@@H](O)c5ccccc5-c5ccc(Cl)cc5)CC4)cc3)cc2S(=O)(=O)C(F)(F)F)CC1]

In some embodiments, BBM is

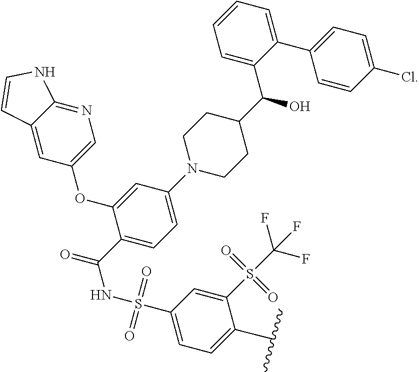 [SMILES: *c1ccc(S(=O)(=O)NC(=O)c2ccc(N3CCC([C@H](O)c4ccccc4-c4ccc(Cl)cc4)CC3)cc2Oc2cnc3[nH]ccc3c2)cc1S(=O)(=O)C(F)(F)F]

In some embodiments, BBM is

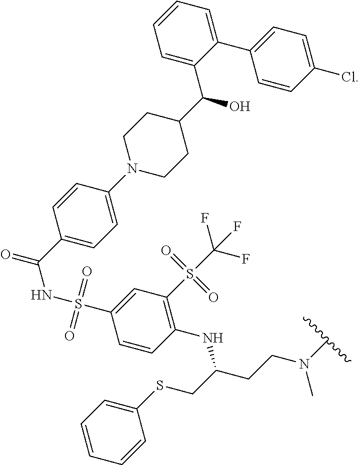 [SMILES: *N(C)CC[C@H](CSc1ccccc1)Nc1ccc(S(=O)(=O)NC(=O)c2ccc(N3CCC([C@H](O)c4ccccc4-c4ccc(Cl)cc4)CC3)cc2)cc1S(=O)(=O)C(F)(F)F]

In some embodiments, BBM is

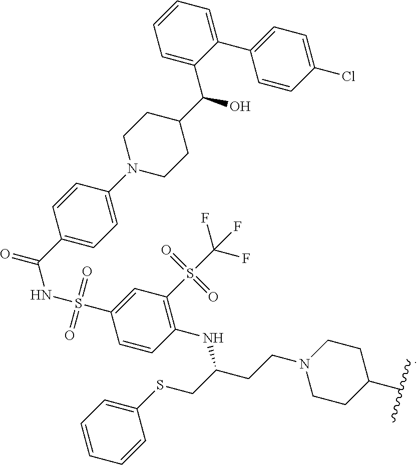 [SMILES: *C1CCN(CC[C@H](CSc2ccccc2)Nc2ccc(S(=O)(=O)NC(=O)c3ccc(N4CCC([C@H](O)c5ccccc5-c5ccc(Cl)cc5)CC4)cc3)cc2S(=O)(=O)C(F)(F)F)CC1]

In some embodiments, BBM is

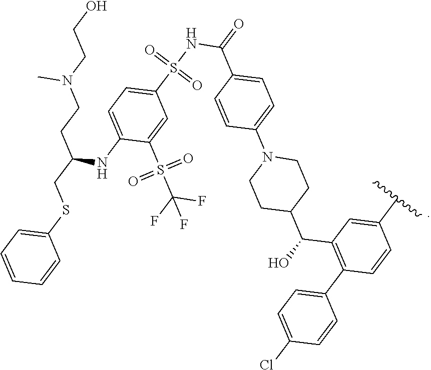 [SMILES: *c1ccc(-c2ccc(Cl)cc2)c([C@H](O)C2CCN(c3ccc(C(=O)NS(=O)(=O)c4ccc(N[C@H](CCN(C)CCO)CSc5ccccc5)c(S(=O)(=O)C(F)(F)F)c4)cc3)CC2)c1]

In some embodiments, BBM is

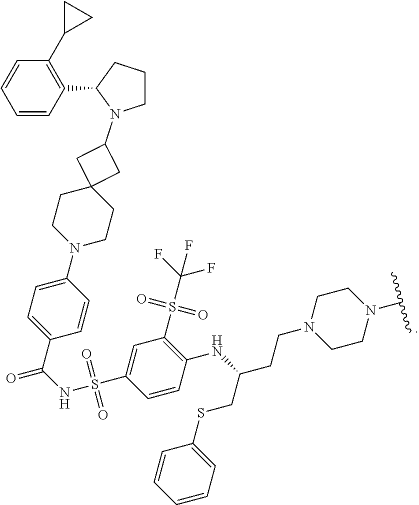 [SMILES: *N1CCN(CC[C@H](CSc2ccccc2)Nc2ccc(S(=O)(=O)NC(=O)c3ccc(N4CCC5(CC4)CC(N4CCC[C@H]4c4ccccc4C4CC4)C5)cc3)cc2S(=O)(=O)C(F)(F)F)CC1]

In some embodiments, BBM is

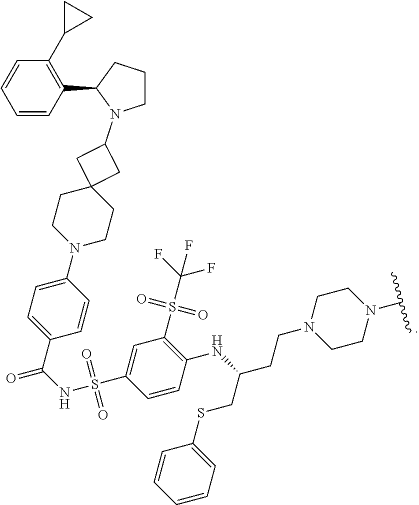 [SMILES: *N1CCN(CC[C@H](CSc2ccccc2)Nc2ccc(S(=O)(=O)NC(=O)c3ccc(N4CCC5(CC4)CC(N4CCC[C@@H]4c4ccccc4C4CC4)C5)cc3)cc2S(=O)(=O)C(F)(F)F)CC1]

In some embodiments, BBM is

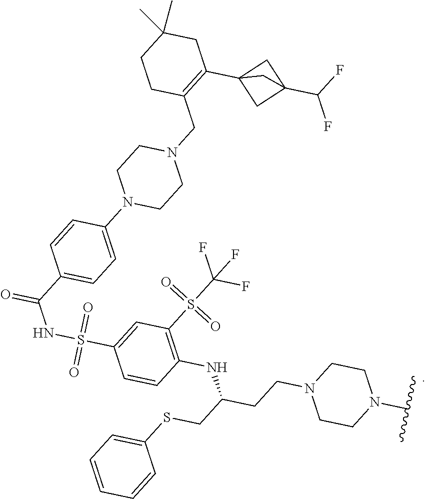 [SMILES: *N1CCN(CC[C@H](CSc2ccccc2)Nc2ccc(S(=O)(=O)NC(=O)c3ccc(N4CCN(CC5=C(C67CC(C(F)F)(C6)C7)CC(C)(C)CC5)CC4)cc3)cc2S(=O)(=O)C(F)(F)F)CC1]

In some embodiments, BBM is

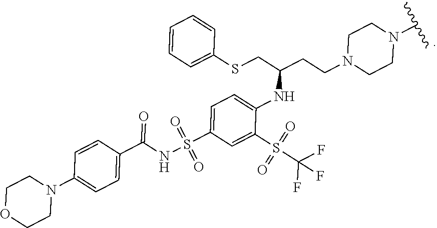 [SMILES: *N1CCN(CC[C@H](CSc2ccccc2)Nc2ccc(S(=O)(=O)NC(=O)c3ccc(N4CCOCC4)cc3)cc2S(=O)(=O)C(F)(F)F)CC1]

In some embodiments, BBM is

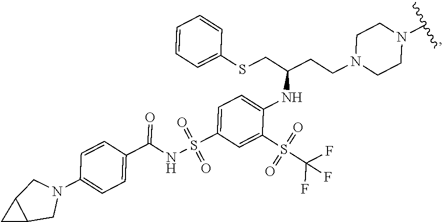 [SMILES: *N1CCN(CC[C@H](CSc2ccccc2)Nc2ccc(S(=O)(=O)NC(=O)c3ccc(N4CC5CC5C4)cc3)cc2S(=O)(=O)C(F)(F)F)CC1]

In some embodiments, BBM is

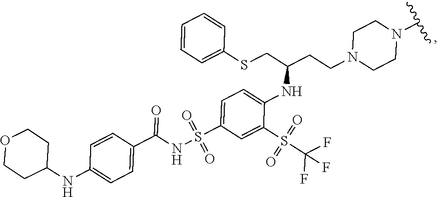 [SMILES: *N1CCN(CC[C@H](CSc2ccccc2)Nc2ccc(S(=O)(=O)NC(=O)c3ccc(NC4CCOCC4)cc3)cc2S(=O)(=O)C(F)(F)F)CC1]

In some embodiments, BBM is

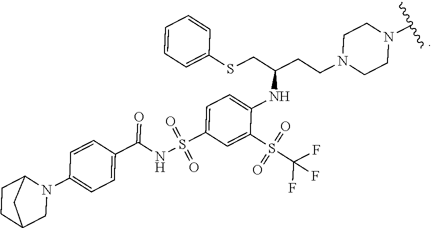 [SMILES: *N1CCN(CC[C@H](CSc2ccccc2)Nc2ccc(S(=O)(=O)NC(=O)c3ccc(N4CC5CCC4C5)cc3)cc2S(=O)(=O)C(F)(F)F)CC1]

In some embodiments, BBM is

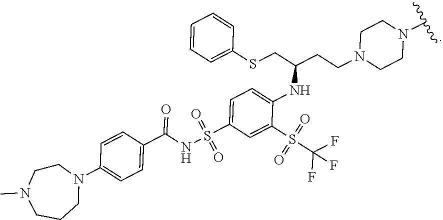 [SMILES: *N1CCN(CC[C@H](CSc2ccccc2)Nc2ccc(S(=O)(=O)NC(=O)c3ccc(N4CCCN(C)CC4)cc3)cc2S(=O)(=O)C(F)(F)F)CC1]

In some embodiments, BBM is

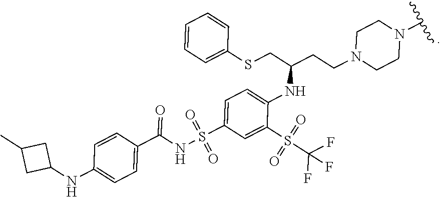 [SMILES: *N1CCN(CC[C@H](CSc2ccccc2)Nc2ccc(S(=O)(=O)NC(=O)c3ccc(NC4CC(C)C4)cc3)cc2S(=O)(=O)C(F)(F)F)CC1]

In some embodiments, BBM is

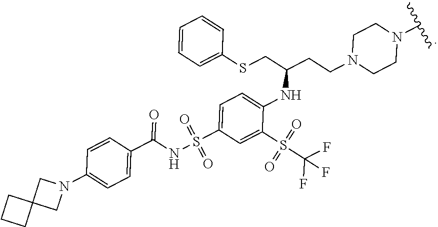 [SMILES: *N1CCN(CC[C@H](CSc2ccccc2)Nc2ccc(S(=O)(=O)NC(=O)c3ccc(N4CC5(CCC5)C4)cc3)cc2S(=O)(=O)C(F)(F)F)CC1]

In some embodiments, BBM is

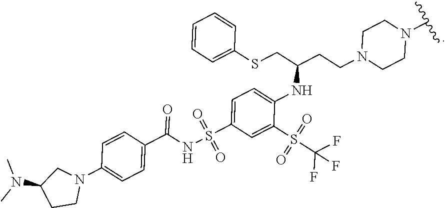 [SMILES: *N1CCN(CC[C@H](CSc2ccccc2)Nc2ccc(S(=O)(=O)NC(=O)c3ccc(N4CC[C@@H](N(C)C)C4)cc3)cc2S(=O)(=O)C(F)(F)F)CC1]

In some embodiments, BBM is

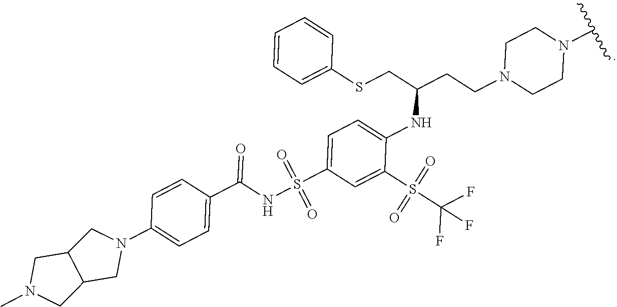 [SMILES: *N1CCN(CC[C@H](CSc2ccccc2)Nc2ccc(S(=O)(=O)NC(=O)c3ccc(N4CC5CN(C)CC5C4)cc3)cc2S(=O)(=O)C(F)(F)F)CC1]

In some embodiments, BBM is

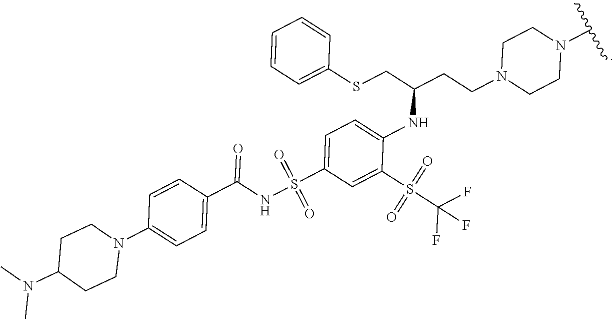 [SMILES: *N1CCN(CC[C@H](CSc2ccccc2)Nc2ccc(S(=O)(=O)NC(=O)c3ccc(N4CCC(N(C)C)CC4)cc3)cc2S(=O)(=O)C(F)(F)F)CC1]

In some embodiments, BBM is

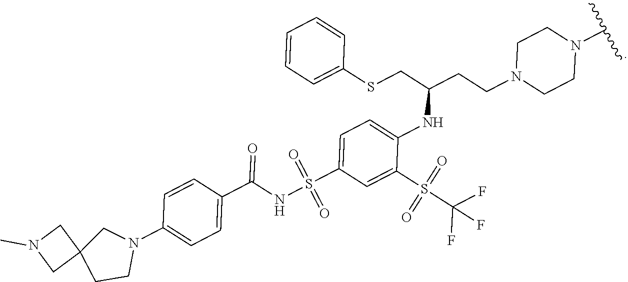 [SMILES: *N1CCN(CC[C@H](CSc2ccccc2)Nc2ccc(S(=O)(=O)NC(=O)c3ccc(N4CCC5(CN(C)C5)C4)cc3)cc2S(=O)(=O)C(F)(F)F)CC1]

In some embodiments, BBM is

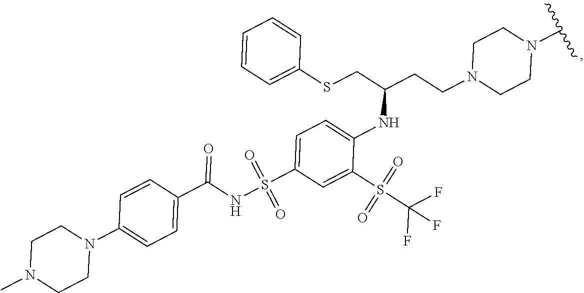 [SMILES: *N1CCN(CC[C@H](CSc2ccccc2)Nc2ccc(S(=O)(=O)NC(=O)c3ccc(N4CCN(C)CC4)cc3)cc2S(=O)(=O)C(F)(F)F)CC1]

In some embodiments, BBM is

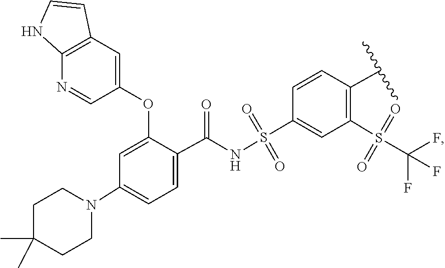 [SMILES: *c1ccc(S(=O)(=O)NC(=O)c2ccc(N3CCC(C)(C)CC3)cc2Oc2cnc3[nH]ccc3c2)cc1S(=O)(=O)C(F)(F)F]

In some embodiments, BBM is

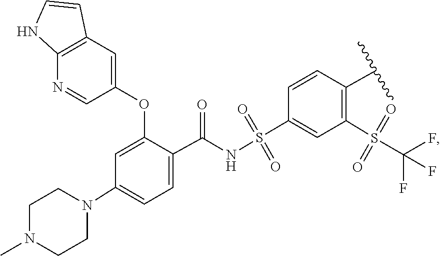 [SMILES: *c1ccc(S(=O)(=O)NC(=O)c2ccc(N3CCN(C)CC3)cc2Oc2cnc3[nH]ccc3c2)cc1S(=O)(=O)C(F)(F)F]

In some embodiments, BBM is

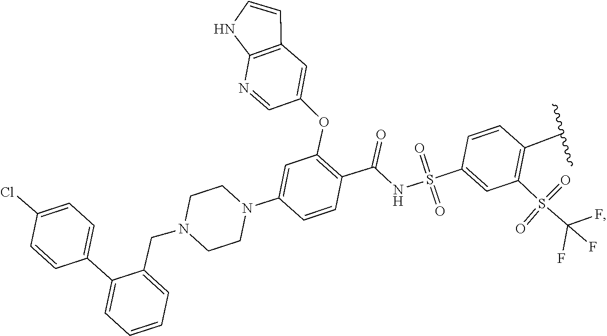 [SMILES: *c1ccc(S(=O)(=O)NC(=O)c2ccc(N3CCN(Cc4ccccc4-c4ccc(Cl)cc4)CC3)cc2Oc2cnc3[nH]ccc3c2)cc1S(=O)(=O)C(F)(F)F]

In some embodiments, BBM is

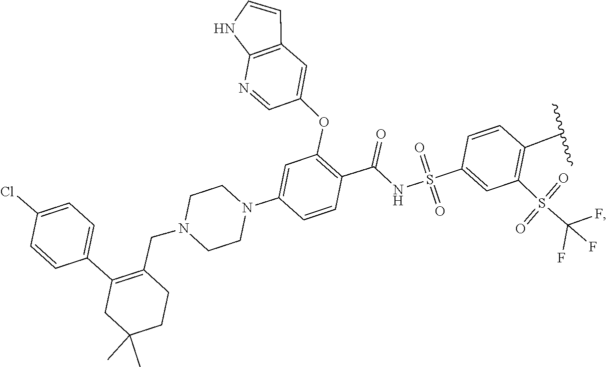 [SMILES: *c1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1S(=O)(=O)C(F)(F)F]

In some embodiments, BBM is

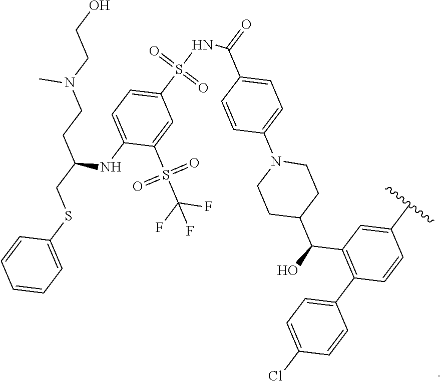 [SMILES: *c1ccc(-c2ccc(Cl)cc2)c([C@@H](O)C2CCN(c3ccc(C(=O)NS(=O)(=O)c4ccc(N[C@H](CCN(C)CCO)CSc5ccccc5)c(S(=O)(=O)C(F)(F)F)c4)cc3)CC2)c1]

In some embodiments, BBM is

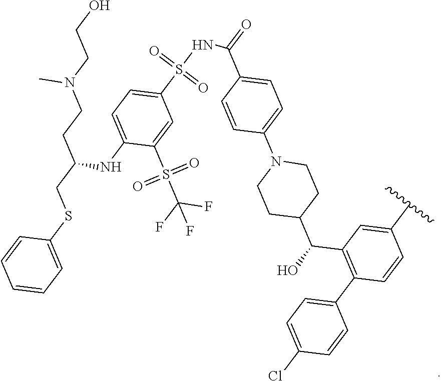 [SMILES: *c1ccc(-c2ccc(Cl)cc2)c([C@H](O)C2CCN(c3ccc(C(=O)NS(=O)(=O)c4ccc(N[C@@H](CCN(C)CCO)CSc5ccccc5)c(S(=O)(=O)C(F)(F)F)c4)cc3)CC2)c1]

In some embodiments, BBM is

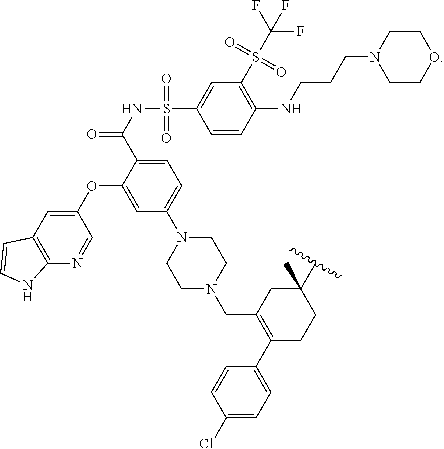 [SMILES: *[C@]1(C)CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1]

In some embodiments, BBM is

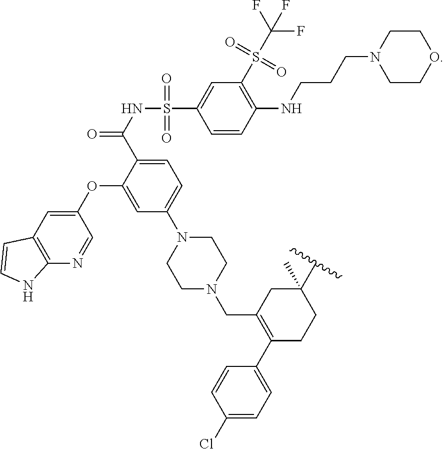 [SMILES: *[C@@]1(C)CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1]

In some embodiments, BBM is

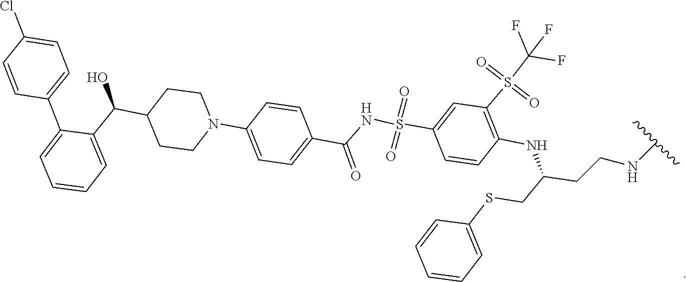 [SMILES: *NCC[C@H](CSc1ccccc1)Nc1ccc(S(=O)(=O)NC(=O)c2ccc(N3CCC([C@H](O)c4ccccc4-c4ccc(Cl)cc4)CC3)cc2)cc1S(=O)(=O)C(F)(F)F]

In some embodiments, BBM is

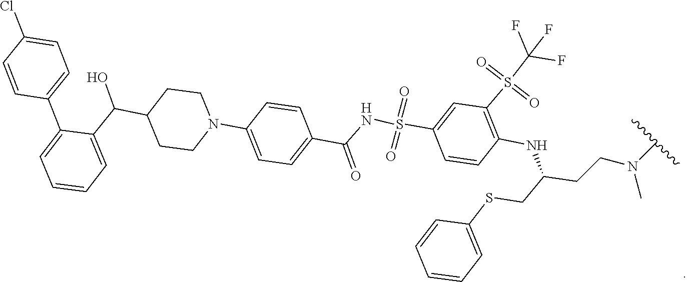 [SMILES: *N(C)CC[C@H](CSc1ccccc1)Nc1ccc(S(=O)(=O)NC(=O)c2ccc(N3CCC(C(O)c4ccccc4-c4ccc(Cl)cc4)CC3)cc2)cc1S(=O)(=O)C(F)(F)F]

In some embodiments, BBM is

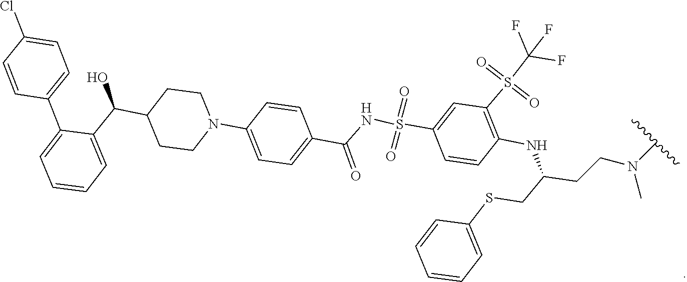 [SMILES: *N(C)CC[C@H](CSc1ccccc1)Nc1ccc(S(=O)(=O)NC(=O)c2ccc(N3CCC([C@H](O)c4ccccc4-c4ccc(Cl)cc4)CC3)cc2)cc1S(=O)(=O)C(F)(F)F]

In some embodiments, BBM is

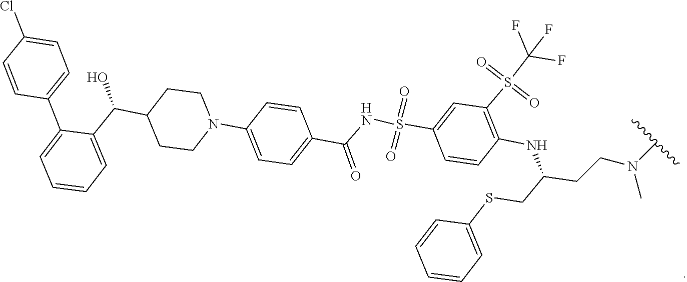 [SMILES: *N(C)CC[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F]

In some embodiments, BBM is

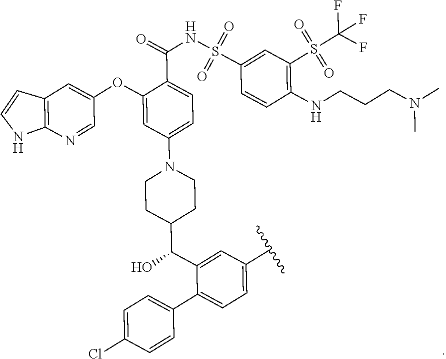 [SMILES: *c1ccc(-c2ccc(Cl)cc2)c([C@H](O)C2CCN(c3ccc(C(=O)NS(=O)(=O)c4ccc(NCCCN(C)C)c(S(=O)(=O)C(F)(F)F)c4)c(Oc4cnc5[nH]ccc5c4)c3)CC2)c1]

In some embodiments, BBM is

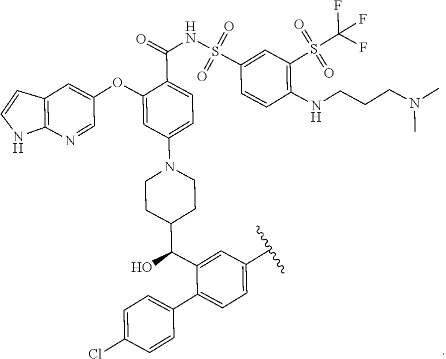 [SMILES: *c1ccc(-c2ccc(Cl)cc2)c([C@@H](O)C2CCN(c3ccc(C(=O)NS(=O)(=O)c4ccc(NCCCN(C)C)c(S(=O)(=O)C(F)(F)F)c4)c(Oc4cnc5[nH]ccc5c4)c3)CC2)c1]

In some embodiments, BBM is

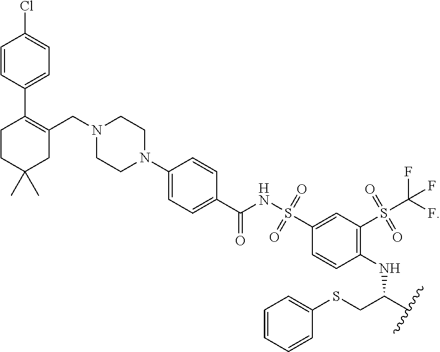 [SMILES: *[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F]

In some embodiments, BBM is

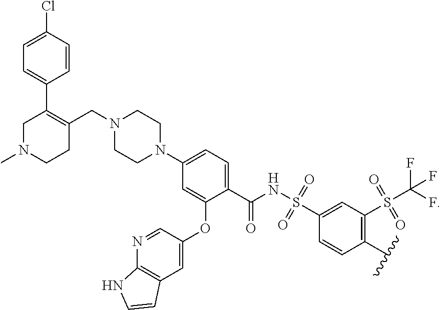 [SMILES: *c1ccc(S(=O)(=O)NC(=O)c2ccc(N3CCN(CC4=C(c5ccc(Cl)cc5)CN(C)CC4)CC3)cc2Oc2cnc3[nH]ccc3c2)cc1S(=O)(=O)C(F)(F)F]

In some embodiments, BBM is

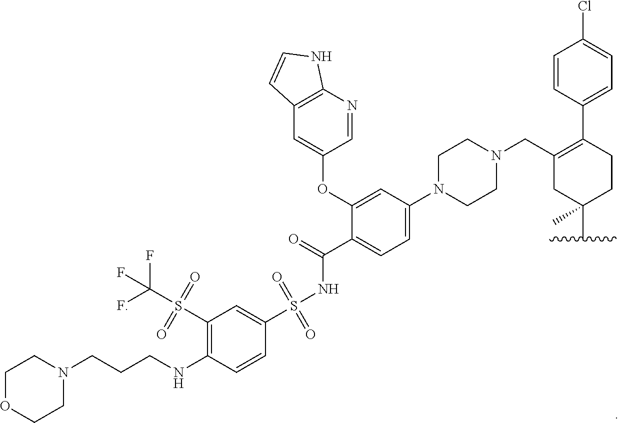 [SMILES: *[C@]1(C)CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1]

In some embodiments, BBM is

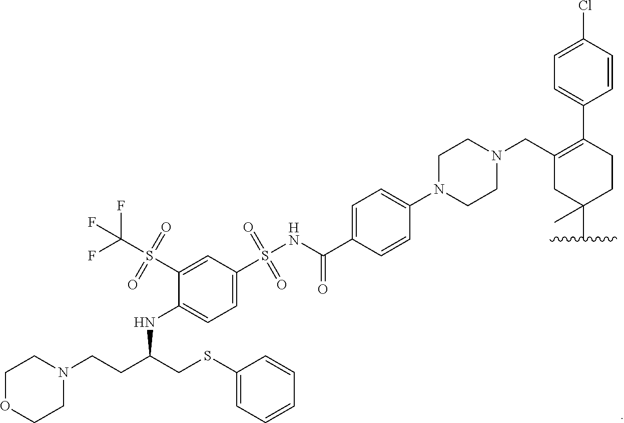 [SMILES: *C1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some embodiments, BBM is

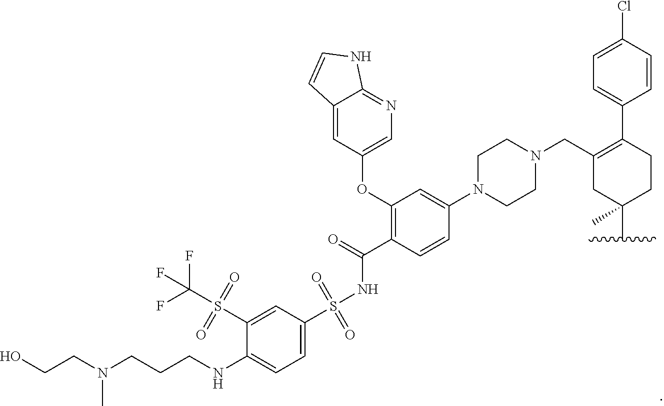 [SMILES: *[C@]1(C)CCC(c2ccc(Cl)cc2)=C(CN2CCN(c3ccc(C(=O)NS(=O)(=O)c4ccc(NCCCN(C)CCO)c(S(=O)(=O)C(F)(F)F)c4)c(Oc4cnc5[nH]ccc5c4)c3)CC2)C1]

In some embodiments, BBM is

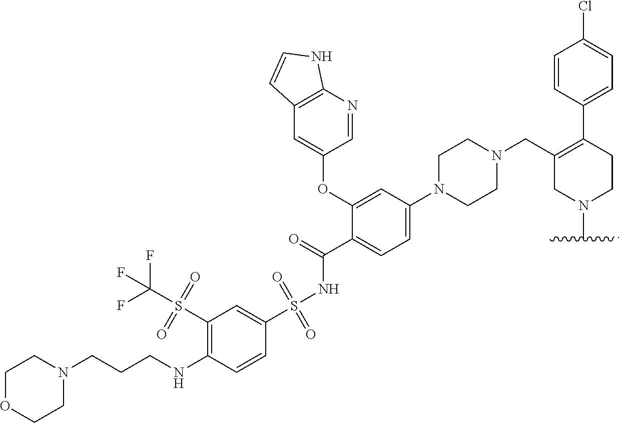 [SMILES: *N1CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1]

In some embodiments, BBM is

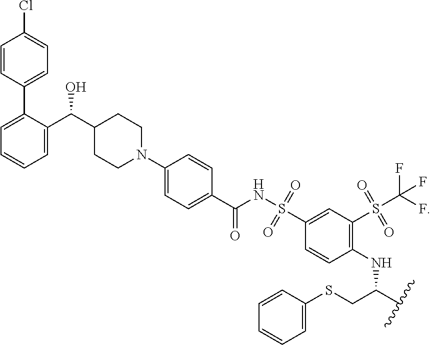 [SMILES: *[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F]

In some embodiments, BBM is

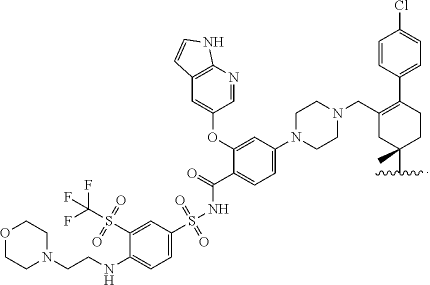 [SMILES: *[C@@]1(C)CCC(c2ccc(Cl)cc2)=C(CN2CCN(c3ccc(C(=O)NS(=O)(=O)c4ccc(NCCN5CCOCC5)c(S(=O)(=O)C(F)(F)F)c4)c(Oc4cnc5[nH]ccc5c4)c3)CC2)C1]

In some embodiments, BBM is

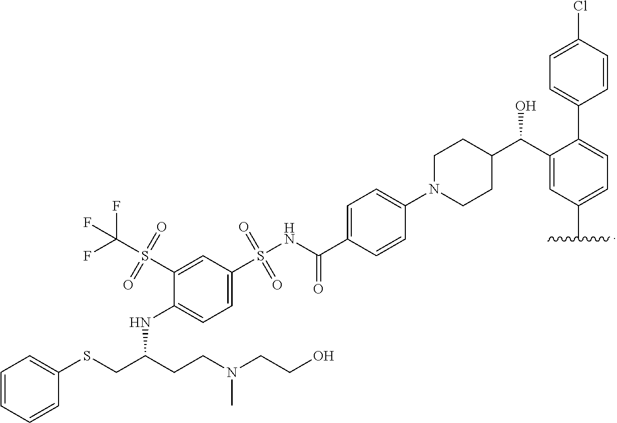 [SMILES: *c1ccc(-c2ccc(Cl)cc2)c([C@@H](O)C2CCN(c3ccc(C(=O)NS(=O)(=O)c4ccc(N[C@H](CCN(C)CCO)CSc5ccccc5)c(S(=O)(=O)C(F)(F)F)c4)cc3)CC2)c1]

In some embodiments, BBM is

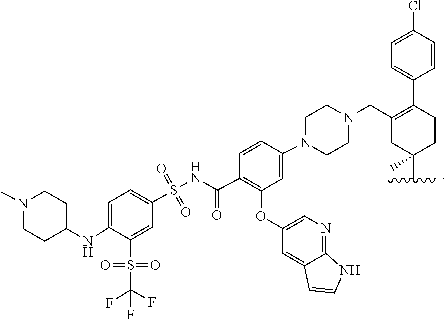 [SMILES: *[C@]1(C)CCC(c2ccc(Cl)cc2)=C(CN2CCN(c3ccc(C(=O)NS(=O)(=O)c4ccc(NC5CCN(C)CC5)c(S(=O)(=O)C(F)(F)F)c4)c(Oc4cnc5[nH]ccc5c4)c3)CC2)C1]

In some embodiments, BBM is

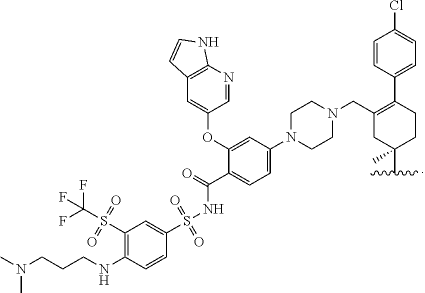 [SMILES: *[C@]1(C)CCC(c2ccc(Cl)cc2)=C(CN2CCN(c3ccc(C(=O)NS(=O)(=O)c4ccc(NCCCN(C)C)c(S(=O)(=O)C(F)(F)F)c4)c(Oc4cnc5[nH]ccc5c4)c3)CC2)C1]

In some embodiments, BBM is

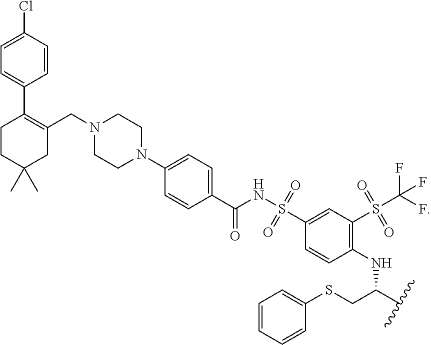 [SMILES: *[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F]

In some embodiments, BBM is

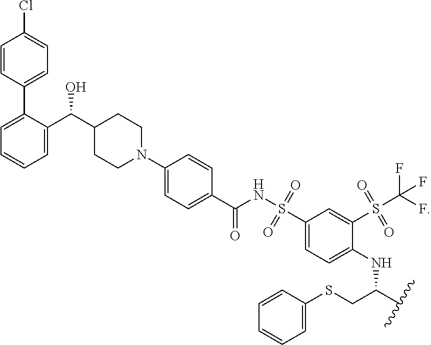 [SMILES: *[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F]

In some embodiments, BBM is

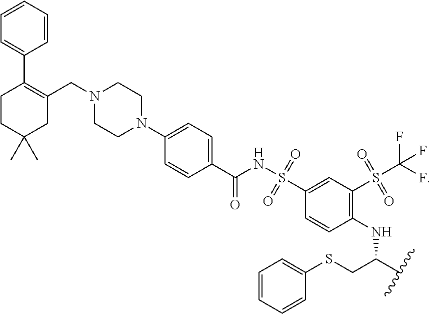 [SMILES: *[C@H](CSc1ccccc1)Nc1ccc(S(=O)(=O)NC(=O)c2ccc(N3CCN(CC4=C(c5ccccc5)CCC(C)(C)C4)CC3)cc2)cc1S(=O)(=O)C(F)(F)F]

In some embodiments, BBM is

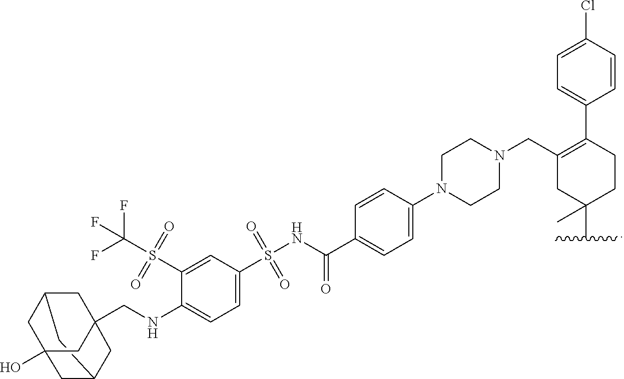 [SMILES: *C1(C)CCC(c2ccc(Cl)cc2)=C(CN2CCN(c3ccc(C(=O)NS(=O)(=O)c4ccc(NCC56CC7CC(CC(O)(C7)C5)C6)c(S(=O)(=O)C(F)(F)F)c4)cc3)CC2)C1]

In some embodiments, BBM is

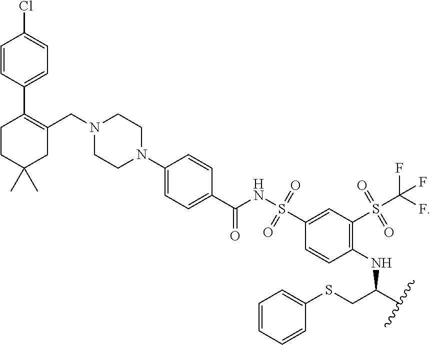 [SMILES: *[C@@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F]

In some embodiments, BBM is

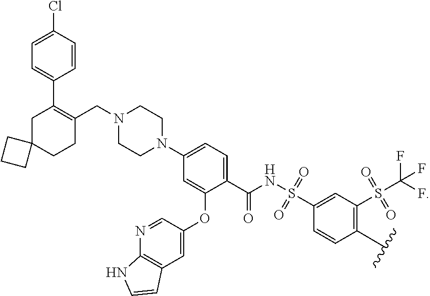 [SMILES: *c1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1S(=O)(=O)C(F)(F)F]

In some embodiments, BBM is

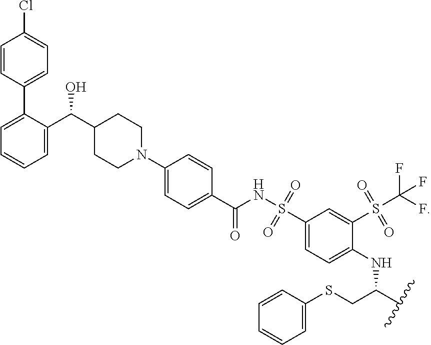 [SMILES: *[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F]

In some embodiments, BBM is

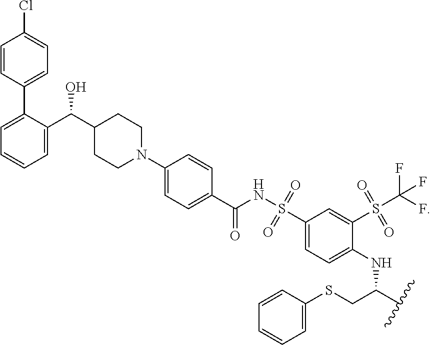 [SMILES: *[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F]

In some embodiments, BBM is

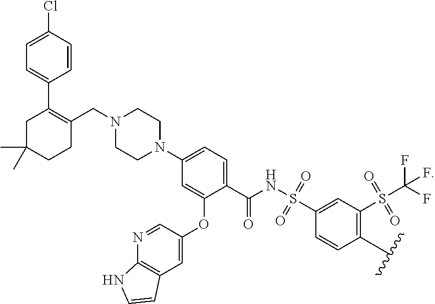 [SMILES: *c1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1S(=O)(=O)C(F)(F)F]

In some embodiments, BBM is

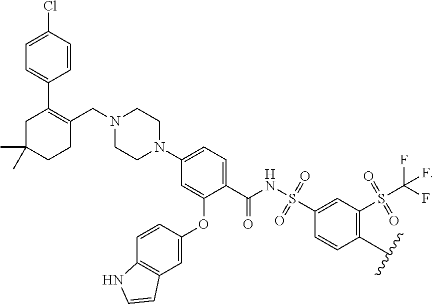 [SMILES: *c1ccc(S(=O)(=O)NC(=O)c2ccc(N3CCN(CC4=C(c5ccc(Cl)cc5)CC(C)(C)CC4)CC3)cc2Oc2ccc3[nH]ccc3c2)cc1S(=O)(=O)C(F)(F)F]

In some embodiments, BBM is

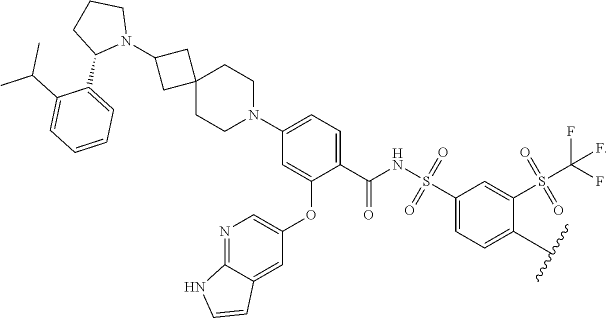 [SMILES: *c1ccc(S(=O)(=O)NC(=O)c2ccc(N3CCC4(CC3)CC(N3CCC[C@H]3c3ccccc3C(C)C)C4)cc2Oc2cnc3[nH]ccc3c2)cc1S(=O)(=O)C(F)(F)F]

In some embodiments, BBM is

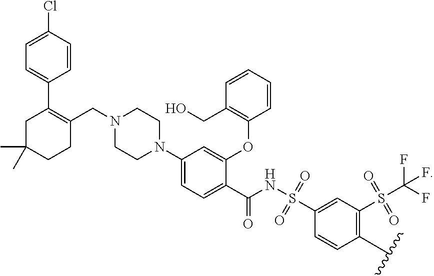 [SMILES: *c1ccc(S(=O)(=O)NC(=O)c2ccc(N3CCN(CC4=C(c5ccc(Cl)cc5)CC(C)(C)CC4)CC3)cc2Oc2ccccc2CO)cc1S(=O)(=O)C(F)(F)F]

In some embodiments, BBM is

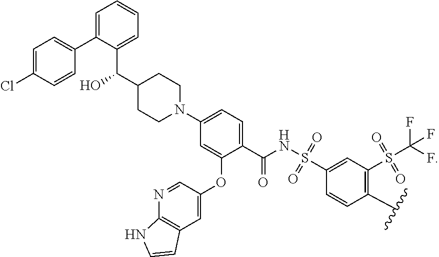 [SMILES: *c1ccc(S(=O)(=O)NC(=O)c2ccc(N3CCC([C@H](O)c4ccccc4-c4ccc(Cl)cc4)CC3)cc2Oc2cnc3[nH]ccc3c2)cc1S(=O)(=O)C(F)(F)F]

In some embodiments, BBM is

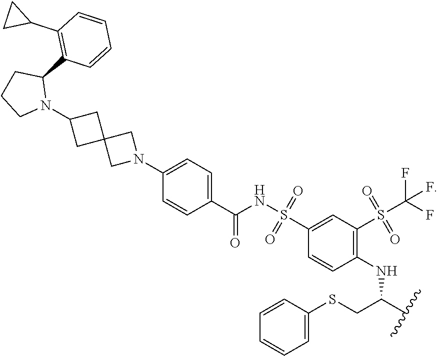 [SMILES: *[C@H](CSc1ccccc1)Nc1ccc(S(=O)(=O)NC(=O)c2ccc(N3CC4(CC(N5CCC[C@H]5c5ccccc5C5CC5)C4)C3)cc2)cc1S(=O)(=O)C(F)(F)F]

In some embodiments, BBM is

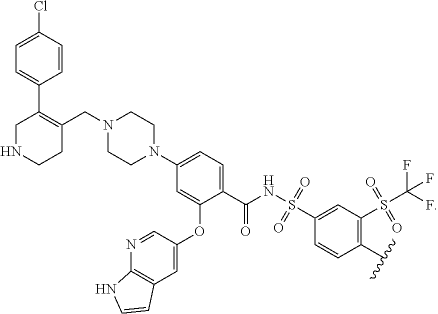 [SMILES: *c1ccc(S(=O)(=O)NC(=O)c2ccc(N3CCN(CC4=C(c5ccc(Cl)cc5)CNCC4)CC3)cc2Oc2cnc3[nH]ccc3c2)cc1S(=O)(=O)C(F)(F)F]

In some embodiments, BBM is

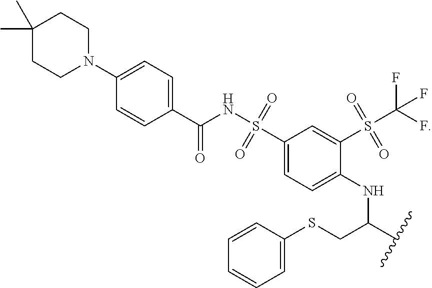 [SMILES: *C(CSc1ccccc1)Nc1ccc(S(=O)(=O)NC(=O)c2ccc(N3CCC(C)(C)CC3)cc2)cc1S(=O)(=O)C(F)(F)F]

In some embodiments, BBM is

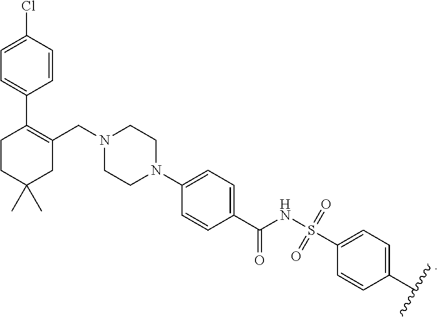 [SMILES: *c1ccc(S(=O)(=O)NC(=O)c2ccc(N3CCN(CC4=C(c5ccc(Cl)cc5)CCC(C)(C)C4)CC3)cc2)cc1]

In some embodiments, BBM is

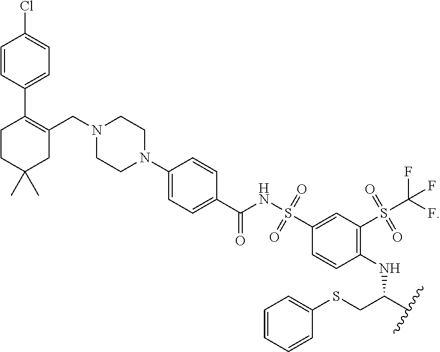 [SMILES: *[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F]

In some embodiments, BBM is

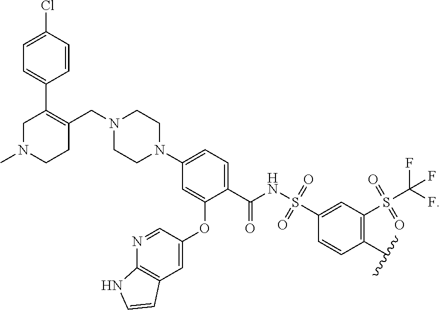 [SMILES: *c1ccc(S(=O)(=O)NC(=O)c2ccc(N3CCN(CC4=C(c5ccc(Cl)cc5)CN(C)CC4)CC3)cc2Oc2cnc3[nH]ccc3c2)cc1S(=O)(=O)C(F)(F)F]

In some embodiments, BBM is

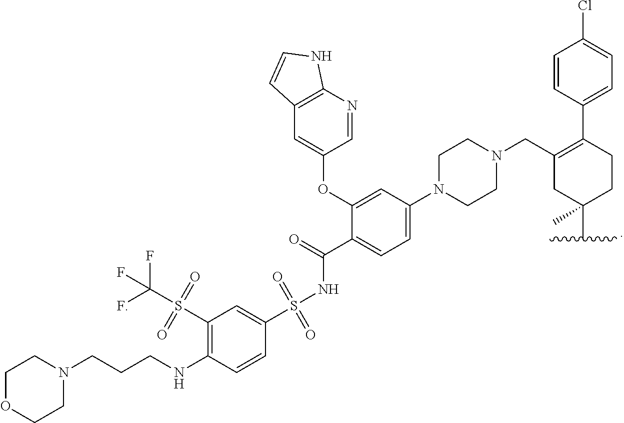 [SMILES: *[C@]1(C)CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1]

In some embodiments, BBM is

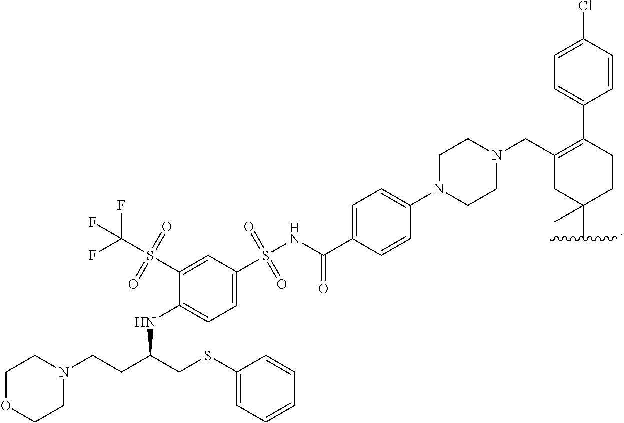 [SMILES: *C1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1]

In some embodiments, BBM is

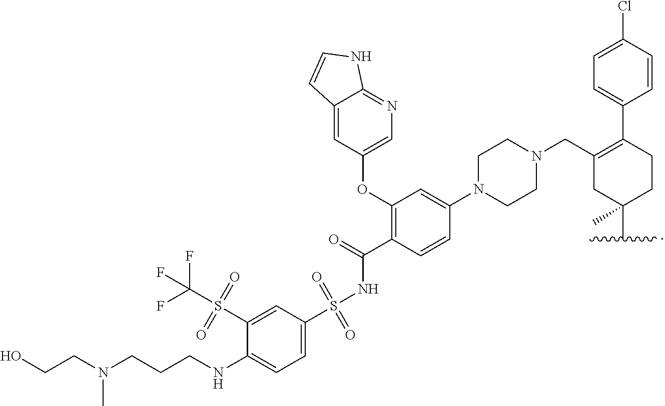 [SMILES: *[C@]1(C)CCC(c2ccc(Cl)cc2)=C(CN2CCN(c3ccc(C(=O)NS(=O)(=O)c4ccc(NCCCN(C)CCO)c(S(=O)(=O)C(F)(F)F)c4)c(Oc4cnc5[nH]ccc5c4)c3)CC2)C1]

In some embodiments, BBM is

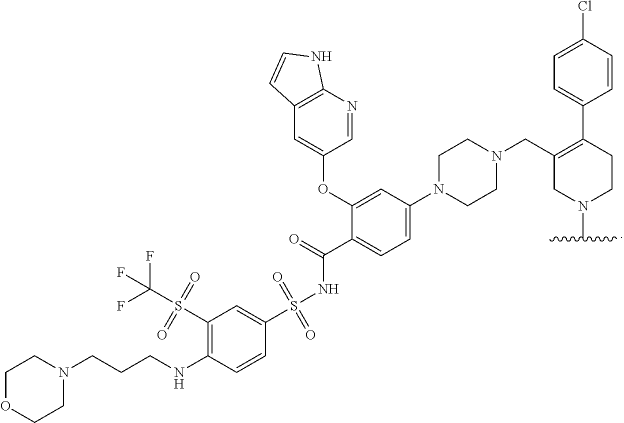 [SMILES: *N1CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1]

In some embodiments, BBM is

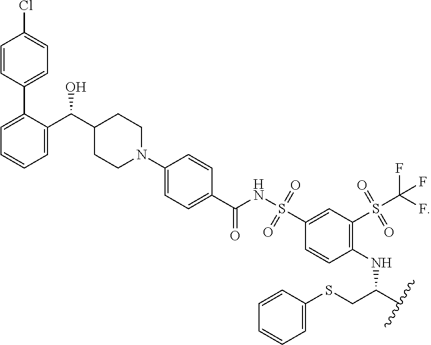 [SMILES: *[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F]

In some embodiments, BBM is

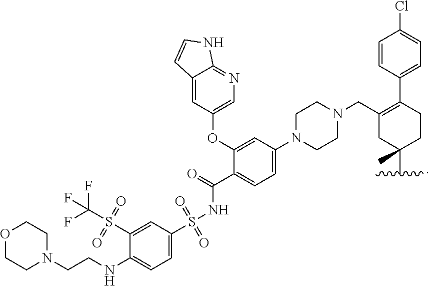 [SMILES: *[C@@]1(C)CCC(c2ccc(Cl)cc2)=C(CN2CCN(c3ccc(C(=O)NS(=O)(=O)c4ccc(NCCN5CCOCC5)c(S(=O)(=O)C(F)(F)F)c4)c(Oc4cnc5[nH]ccc5c4)c3)CC2)C1]

In some embodiments, BBM is

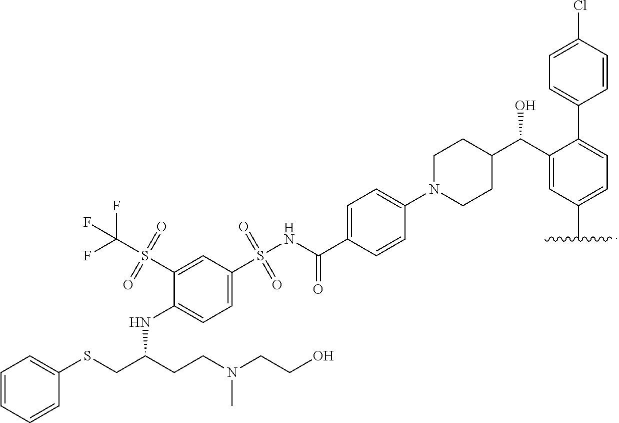 [SMILES: *c1ccc(-c2ccc(Cl)cc2)c([C@@H](O)C2CCN(c3ccc(C(=O)NS(=O)(=O)c4ccc(N[C@H](CCN(C)CCO)CSc5ccccc5)c(S(=O)(=O)C(F)(F)F)c4)cc3)CC2)c1]

In some embodiments, BBM is

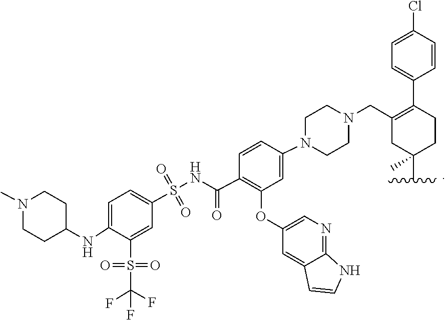 [SMILES: *[C@]1(C)CCC(c2ccc(Cl)cc2)=C(CN2CCN(c3ccc(C(=O)NS(=O)(=O)c4ccc(NC5CCN(C)CC5)c(S(=O)(=O)C(F)(F)F)c4)c(Oc4cnc5[nH]ccc5c4)c3)CC2)C1]

In some embodiments, BBM is

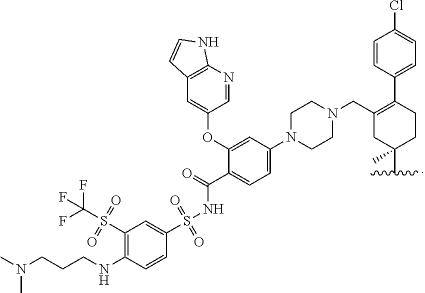 [SMILES: *[C@]1(C)CCC(c2ccc(Cl)cc2)=C(CN2CCN(c3ccc(C(=O)NS(=O)(=O)c4ccc(NCCCN(C)C)c(S(=O)(=O)C(F)(F)F)c4)c(Oc4cnc5[nH]ccc5c4)c3)CC2)C1]

In some embodiments, BBM is

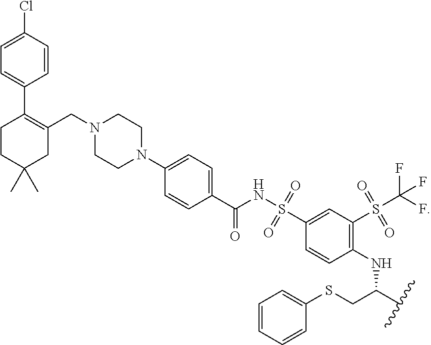 [SMILES: *[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F]

In some embodiments, BBM is

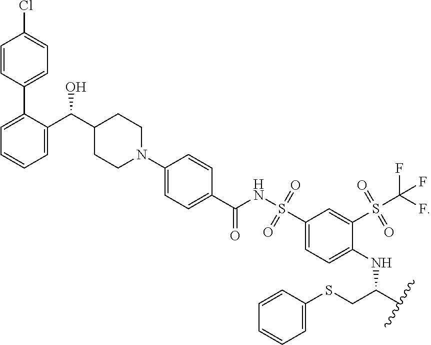 [SMILES: *[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F]

In some embodiments, BBM is

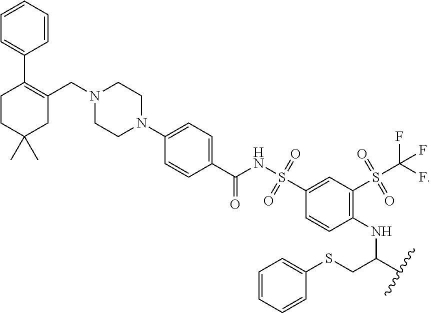 [SMILES: *C(CSc1ccccc1)Nc1ccc(S(=O)(=O)NC(=O)c2ccc(N3CCN(CC4=C(c5ccccc5)CCC(C)(C)C4)CC3)cc2)cc1S(=O)(=O)C(F)(F)F]

In some embodiments, BBM is

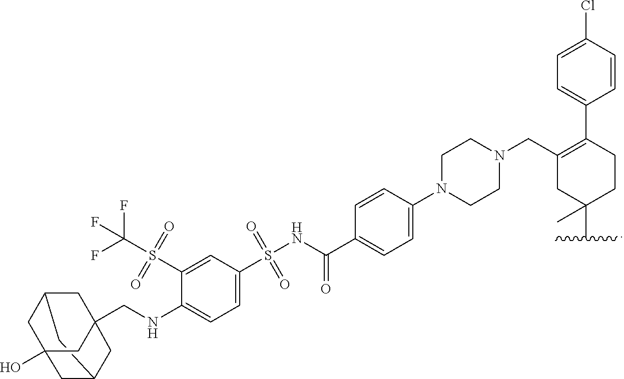 [SMILES: *C1(C)CCC(c2ccc(Cl)cc2)=C(CN2CCN(c3ccc(C(=O)NS(=O)(=O)c4ccc(NCC56CC7CC(CC(O)(C7)C5)C6)c(S(=O)(=O)C(F)(F)F)c4)cc3)CC2)C1]

In some embodiments, BBM is

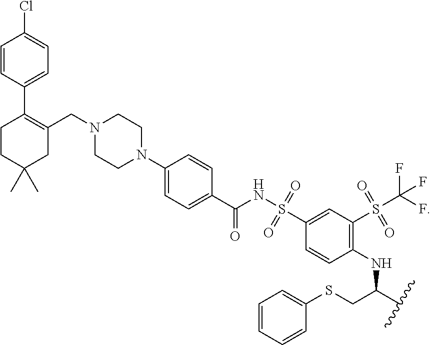 [SMILES: *[C@@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F]

In some embodiments, BBM is

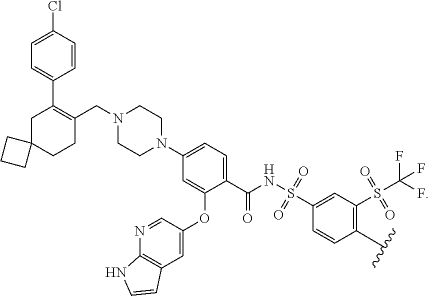 [SMILES: *c1ccc(S(=O)(=O)NC(=O)c2ccc(N3CCN(CC4=C(c5ccc(Cl)cc5)CC5(CCC5)CC4)CC3)cc2Oc2cnc3[nH]ccc3c2)cc1S(=O)(=O)C(F)(F)F]

In some embodiments, BBM is

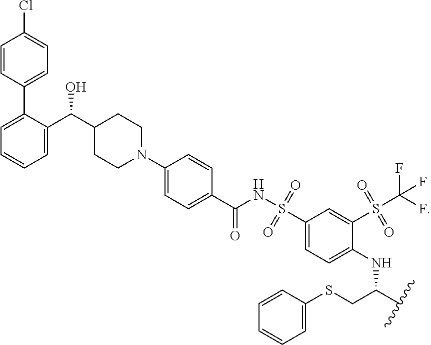 [SMILES: *[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F]

In some embodiments, BBM is

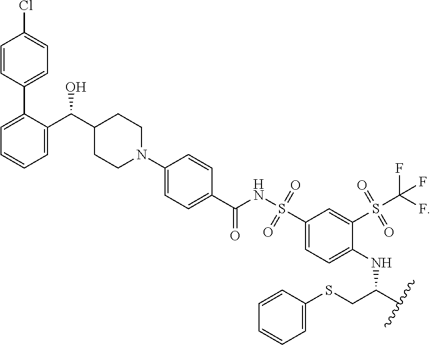 [SMILES: *[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F]

In some embodiments, BBM is

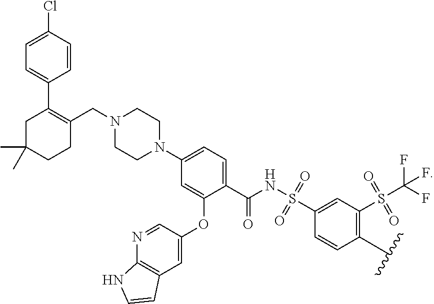 [SMILES: *c1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1S(=O)(=O)C(F)(F)F]

In some embodiments, BBM is

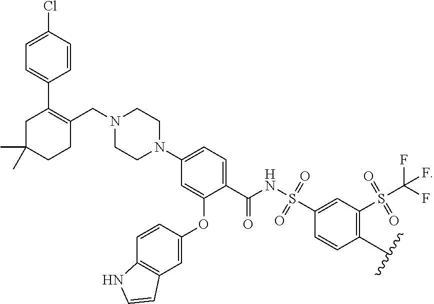 [SMILES: *c1ccc(S(=O)(=O)NC(=O)c2ccc(N3CCN(CC4=C(c5ccc(Cl)cc5)CC(C)(C)CC4)CC3)cc2Oc2ccc3[nH]ccc3c2)cc1S(=O)(=O)C(F)(F)F]

In some embodiments, BBM is

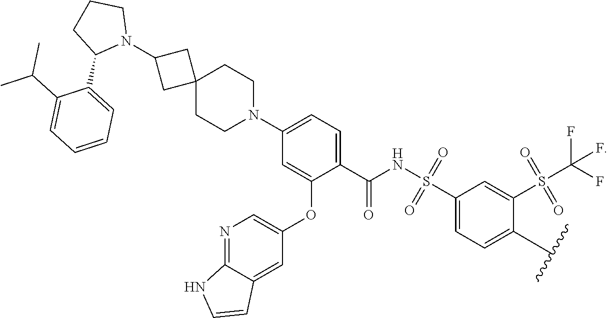 [SMILES: *c1ccc(S(=O)(=O)NC(=O)c2ccc(N3CCC4(CC3)CC(N3CCC[C@H]3c3ccccc3C(C)C)C4)cc2Oc2cnc3[nH]ccc3c2)cc1S(=O)(=O)C(F)(F)F]

In some embodiments, BBM is

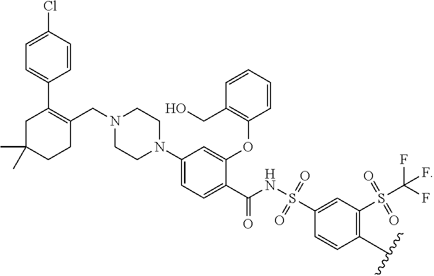 [SMILES: *c1ccc(S(=O)(=O)NC(=O)c2ccc(N3CCN(CC4=C(c5ccc(Cl)cc5)CC(C)(C)CC4)CC3)cc2Oc2ccccc2CO)cc1S(=O)(=O)C(F)(F)F]

In some embodiments, BBM is

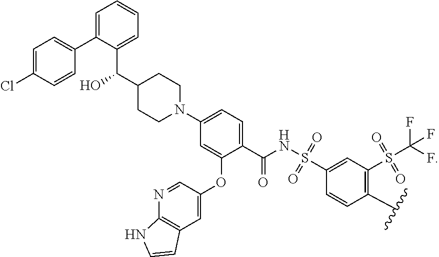 [SMILES: *c1ccc(S(=O)(=O)NC(=O)c2ccc(N3CCC([C@H](O)c4ccccc4-c4ccc(Cl)cc4)CC3)cc2Oc2cnc3[nH]ccc3c2)cc1S(=O)(=O)C(F)(F)F]

In some embodiments, BBM is

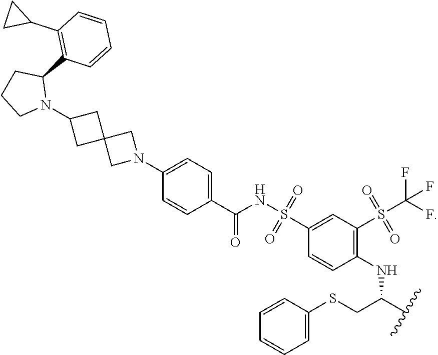 [SMILES: *[C@H](CSc1ccccc1)Nc1ccc(S(=O)(=O)NC(=O)c2ccc(N3CC4(CC(N5CCC[C@H]5c5ccccc5C5CC5)C4)C3)cc2)cc1S(=O)(=O)C(F)(F)F]

In some embodiments, BBM is

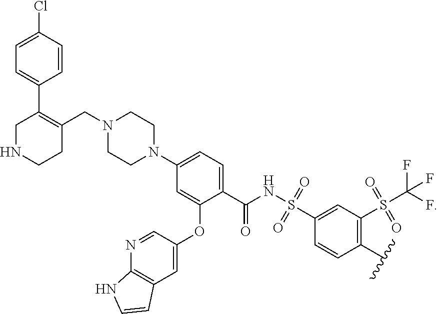 [SMILES: *c1ccc(S(=O)(=O)NC(=O)c2ccc(N3CCN(CC4=C(c5ccc(Cl)cc5)CNCC4)CC3)cc2Oc2cnc3[nH]ccc3c2)cc1S(=O)(=O)C(F)(F)F]

In some embodiments, BBM is

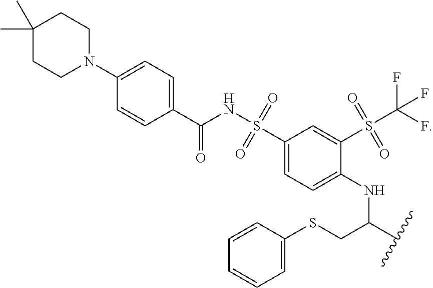 [SMILES: *C(CSc1ccccc1)Nc1ccc(S(=O)(=O)NC(=O)c2ccc(N3CCC(C)(C)CC3)cc2)cc1S(=O)(=O)C(F)(F)F]

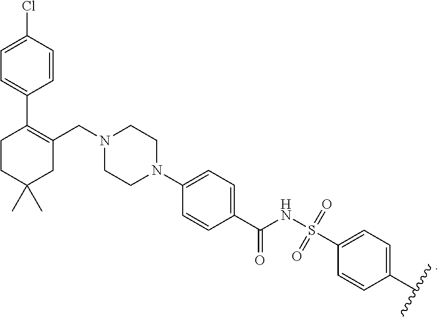 [SMILES: *c1ccc(S(=O)(=O)NC(=O)c2ccc(N3CCN(CC4=C(c5ccc(Cl)cc5)CCC(C)(C)C4)CC3)cc2)cc1]

### Ligase Binding Moiety (LBM)

In some embodiments, LBM is an E3 ligase ligand. Such E3 ligase ligands are well known to one of ordinary skill in the art and include those described in M. Toure, C. M. Crews, Angew. Chem. Int. Ed. 2016, 55, 1966, T. Uehara et al. Nature Chemical Biology 2017, 13, 675, WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, WO 2017/197046, WO 2017/197051, WO 2017/197055, and WO 2017/197056, the entirety of each of which is herein incorporated by reference.

As defined herein and described below, wherein a formula is depicted using square brackets, e.g,

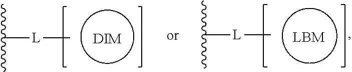

L is attached to a modifiable carbon, oxygen, or nitrogen atom within DIM or LBM including substitution or replacement of a defined group in DIM or LBM.

In certain embodiments, the present invention provides a compound of formula I, wherein LBM is an IMiD-based (immunomodulatory imide drug-based) cereblon E3 ubiquitin ligase binding moiety thereby forming a compound of formula I-a-1, I-a-2, I-a-3, I-a-4, I-a-5, I-a-6, I-a-7, I-a-8, I-a-9, or I-a-10 respectively:

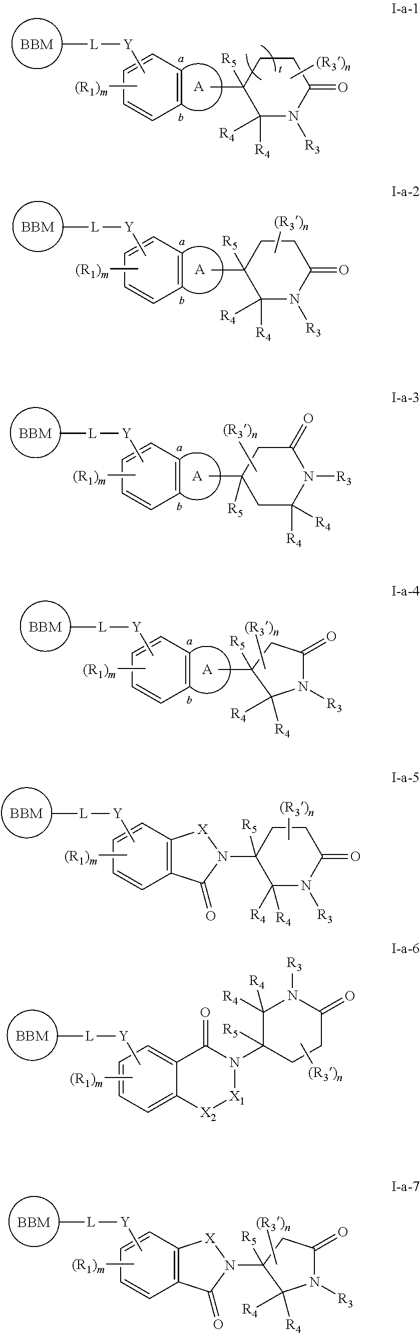

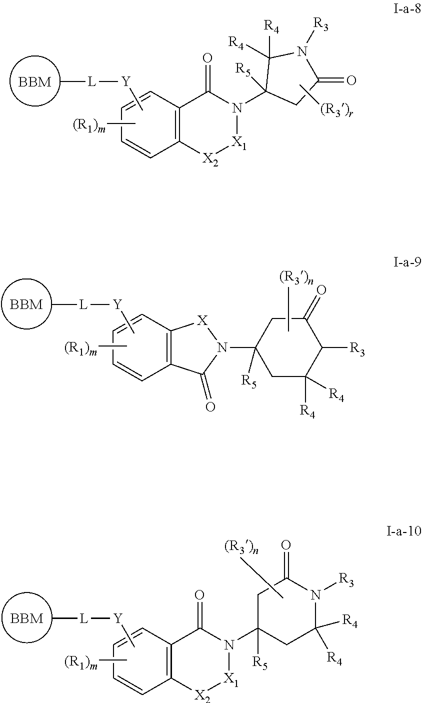

or a compound of formula I-a′-1, I-a′-2, I-a′-3, I-a′-4, I-a′-5, I-a′-6, I-a′-7, I-a′-8, I-a′-9, or I-a′-10 respectively:

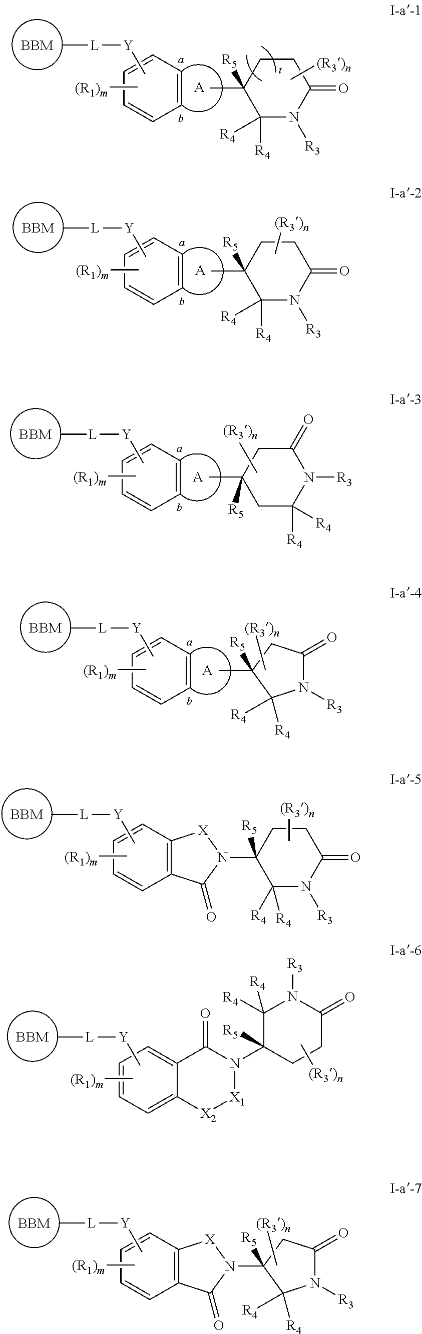

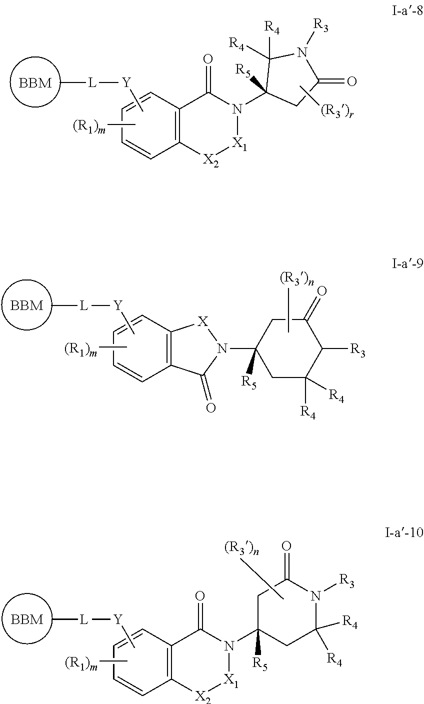

or a compound of formula I-a″-1, I-a″-2, I-a″-3, I-a″-4, I-a″-5, I-a″-6, I-a″-7, I-a″-8, I-a″-9, or I-a″-10 respectively:

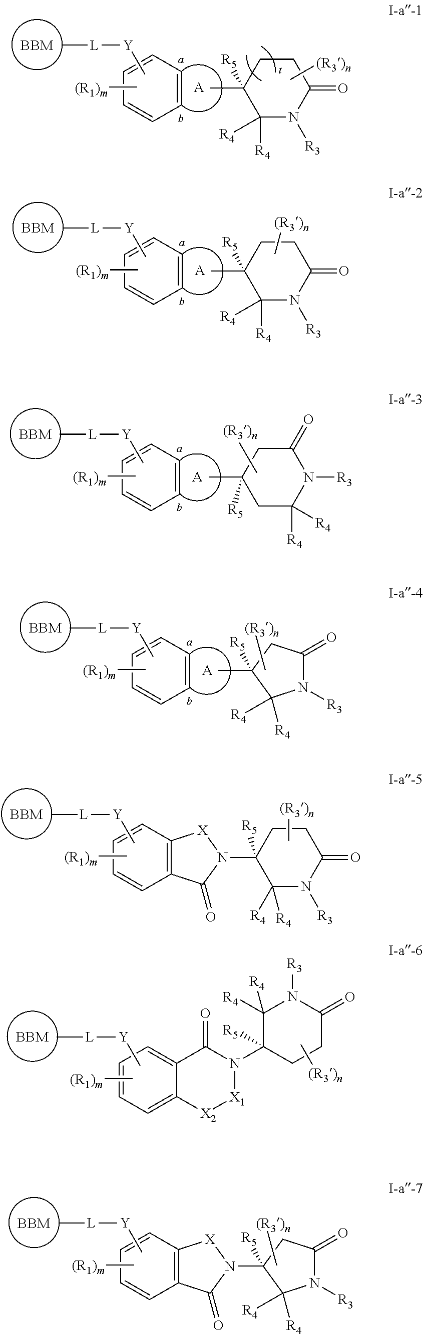

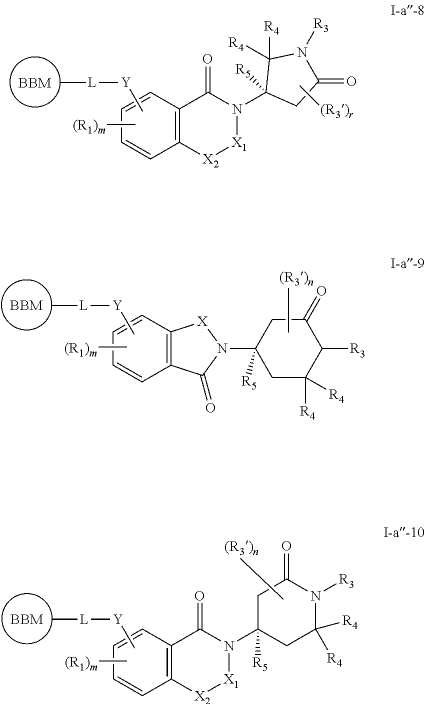

or a pharmaceutically acceptable salt thereof, wherein L and BBM are as defined above and described in embodiments herein, and wherein:

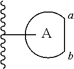

is

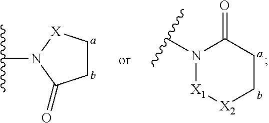

Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R₃)₂;
X₁—X₂ is C(R₃)═N or C(R₃)₂—C(R₃)₂;
each R₁ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆ alkenyl, C(O)—C₃-C₈cycloalkyl, or C(O)-3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
R₂′ is H, C₁-C₆ alkyl, C₂-C₆ alkenyl, C₃-C₈ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
each R₃′ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or Cl;
each Rₐ independently is H or C₁-C₆ alkyl;
R_{b} is H or tosyl;
t is 0 or 1;
m is 0, 1, 2 or 3; and
n is 0, 1 or 2.

In some embodiments, LBM is

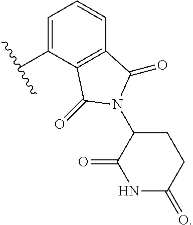 [SMILES: *c1cccc2c1C(=O)N(C1CCC(=O)NC1=O)C2=O]

In some embodiments, LBM is

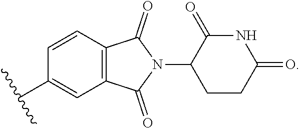 [SMILES: *c1ccc2c(c1)C(=O)N(C1CCC(=O)NC1=O)C2=O]

In some embodiments, LBM is

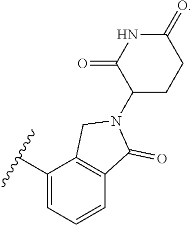 [SMILES: *c1cccc2c1CN(C1CCC(=O)NC1=O)C2=O]

In some embodiments, LBM is

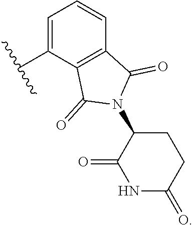 [SMILES: *c1cccc2c1C(=O)N([C@H]1CCC(=O)NC1=O)C2=O]

In some embodiments, LBM is

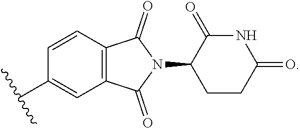 [SMILES: *c1ccc2c(c1)C(=O)N([C@@H]1CCC(=O)NC1=O)C2=O]

In some embodiments, LBM is

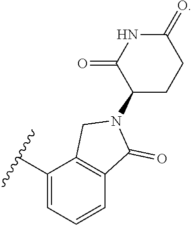 [SMILES: *c1cccc2c1CN([C@@H]1CCC(=O)NC1=O)C2=O]

In some embodiments, LBM is

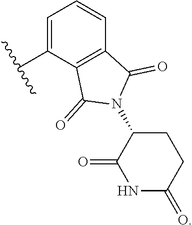 [SMILES: *c1cccc2c1C(=O)N([C@@H]1CCC(=O)NC1=O)C2=O]

In some embodiments, LBM is

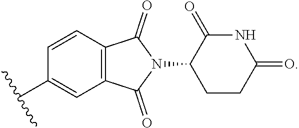 [SMILES: *c1ccc2c(c1)C(=O)N([C@H]1CCC(=O)NC1=O)C2=O]

In some embodiments, LBM is

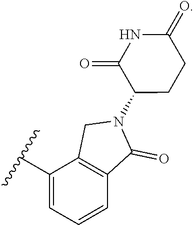 [SMILES: *c1cccc2c1CN([C@H]1CCC(=O)NC1=O)C2=O]

In certain embodiments, the present invention provides a compound of formula I, wherein LBM is a cereblon E3 ubiquitin ligase binding moiety thereby forming a compound of formula I-b:

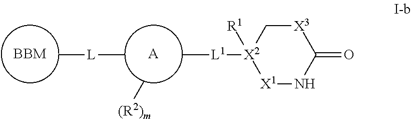

or a pharmaceutically acceptable salt thereof, wherein L and BBM are as defined above and described herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

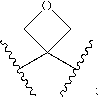 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a bi- or tricyclic ring selected from

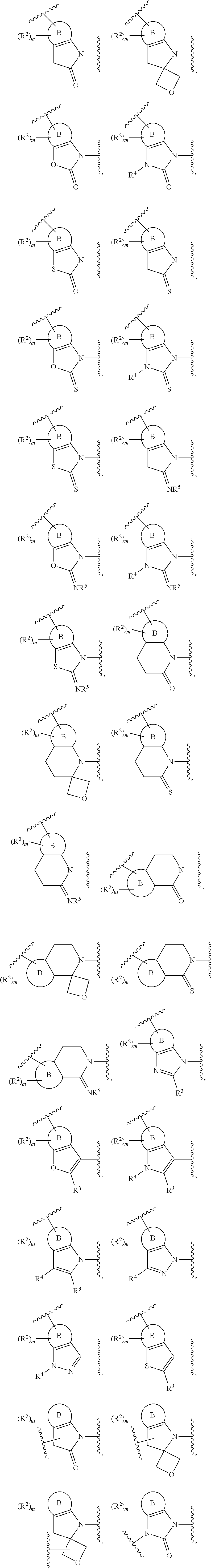

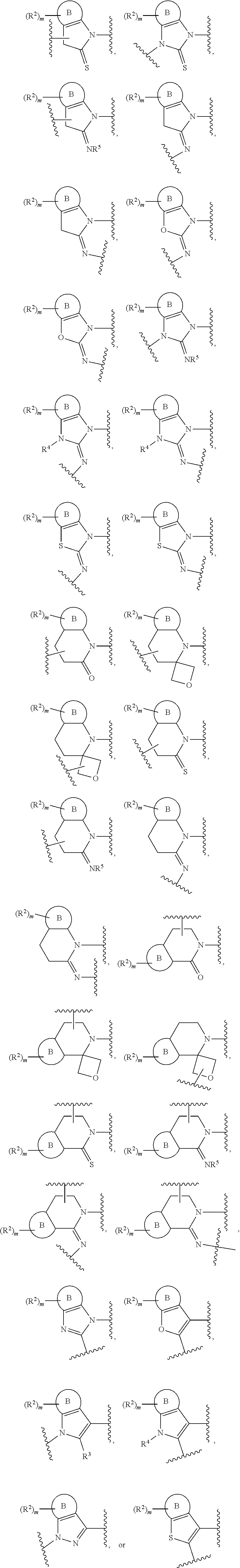

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

Where a point of attachment of —(R²)ₘ is depicted on Ring B, it is intended, and one of ordinary skill in the art would appreciate, that the point of attachment of —(R²)ₘ may be on Ring A and may also be at any available carbon or nitrogen atom on Ring A including the ring to which Ring B is fused. Where —R² is attached to a nitrogen atom bound to R⁴ or R⁵, R⁴ or R⁵ is absent and —R² takes the place of the R⁴ or R⁵ group. Where —R² is attached to a carbon atom bound to R³, R³ is absent and —R² takes the place of the R³ group.

In some embodiments, a compound of formula I-b above is provided as a compound of formula I-b-1 or formula I-b-2:

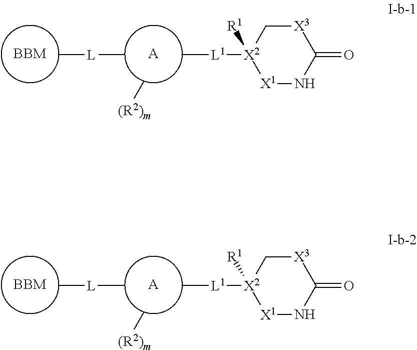

or a pharmaceutically acceptable salt thereof, wherein:
each of BBM, Ring A, L, L¹, R¹, R², X¹, X², X³, and m is as defined above.

In some embodiments, a compound of formula I-b above is provided as a compound of formula I-b-3:

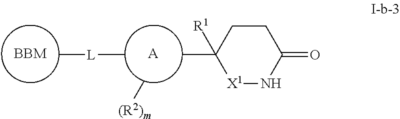

or a pharmaceutically acceptable salt thereof, wherein:
each of BBM, Ring A, L, R¹, R², X¹, and m is as defined above.

In certain embodiments, the present invention provides a compound of formula I, wherein LBM is a cereblon E3 ubiquitin ligase binding moiety thereby forming a compound of formula I-d:

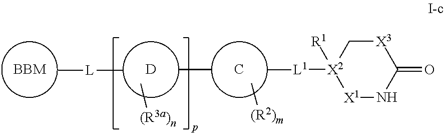

or a pharmaceutically acceptable salt thereof, wherein, L and BBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

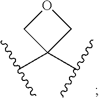 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
Ring C is a mono- or bicyclic ring selected from

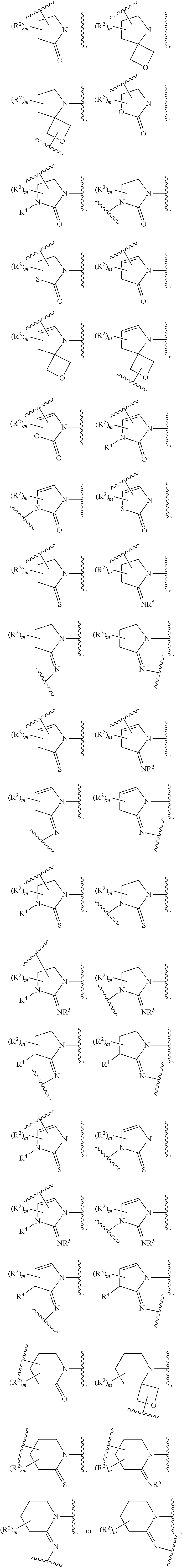

each of R² and R^{3m} is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring D is selected from a 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
n is 0, 1, 2, 3 or 4;
p is 0 or 1, wherein when p is 0, the bond connecting Ring C and Ring D is connected to

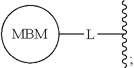

and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-c above is provided as a compound of formula I-c-1 or formula I-c-2:

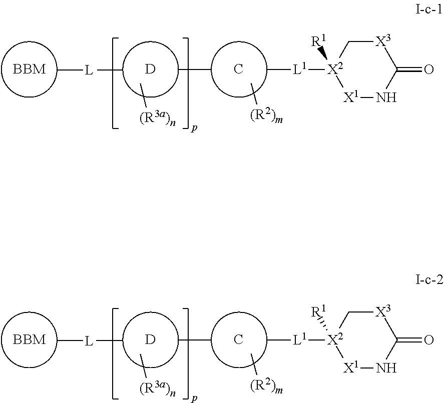

or a pharmaceutically acceptable salt thereof, wherein:
each of BBM, Ring C, Ring D, L, L¹, R¹, R², R^{3a}, X¹, X², X³, n, m, and p is as defined above.

In some embodiments, a compound of formula I-c above is provided as a compound of formula I-c-3:

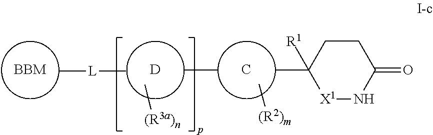

or a pharmaceutically acceptable salt thereof, wherein:
each of BBM, Ring C, Ring D, L, R¹, R², R^{3a}, X¹, n, m, and p is as defined above.

In certain embodiments, the present invention provides a compound of formula I, wherein LBM is a cereblon E3 ubiquitin ligase binding moiety thereby forming a compound of formula I-d:

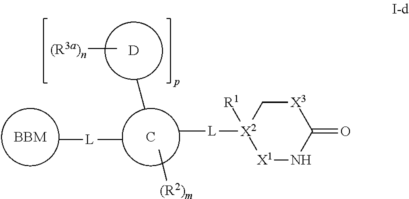

or a pharmaceutically acceptable salt thereof, wherein L and BBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

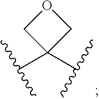 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
Ring C is a mono- or bicyclic ring selected from

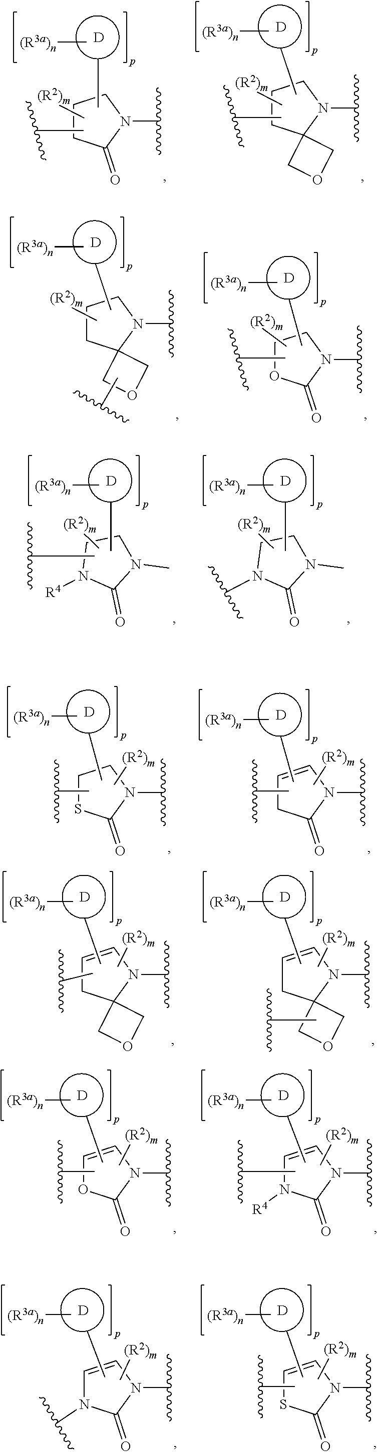

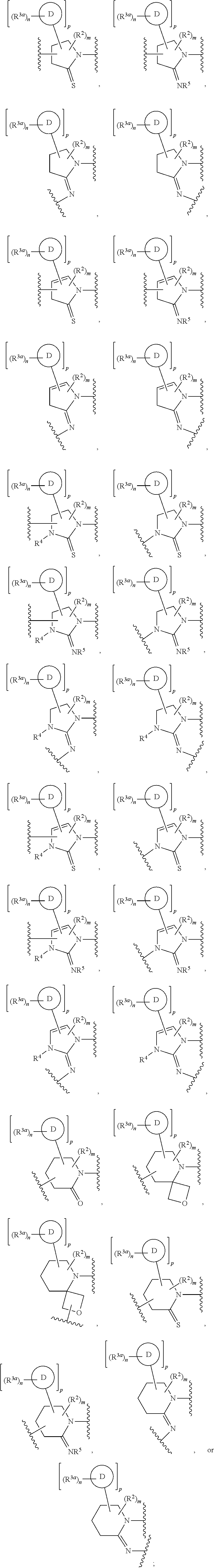

each or R² and R^{3a} is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring D is selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or 4;
n is 0, 1, 2, 3 or 4;
p is 0 or 1; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-d above is provided as a compound of formula I-d-1 or formula I-d-2:

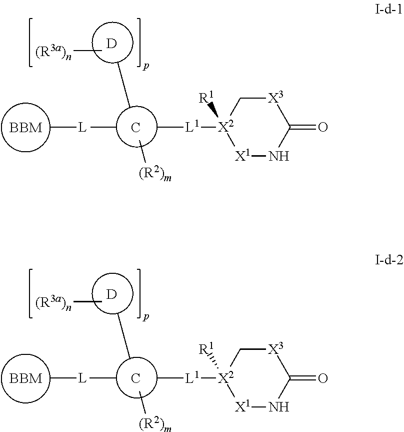

or a pharmaceutically acceptable salt thereof, wherein:
each of BBM, Ring C, Ring D, L, L¹, R¹, R², R^{3a}, X¹, X², X³, m, n, and p is as defined above.

In some embodiments, a compound of formula I-d above is provided as a compound of formula I-d-3:

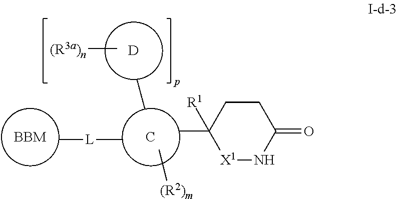

or a pharmaceutically acceptable salt thereof, wherein:
each of BBM, Ring C, Ring D, L, LR¹, R², R^{3a}, X¹, m, n, and p is as defined above.

In certain embodiments, the present invention provides a compound of formula I, wherein LBM is a cereblon E3 ubiquitin ligase binding moiety thereby forming a compound of formula I-e:

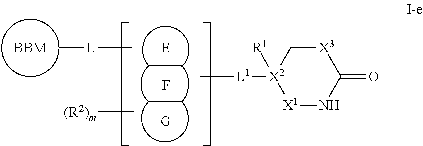

or a pharmaceutically acceptable salt thereof, wherein L and BBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

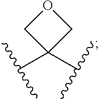 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and Ring G is independently and optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—; and
m is 0, 1, 2, 3, 4, 5, 6, 7, 8, 9, 10, 11, 12, 13, 14, 15, or 16.

Where a point of attachment of

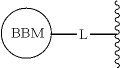

is depicted on Ring E, Ring F, or Ring G, it is intended, and one of ordinary skill in the art would appreciate, that the point of attachment of

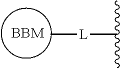

may be on any available carbon or nitrogen atom on Ring E, Ring F, or Ring G, including the ring to which Ring E or Ring G are fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G are fused to Ring F.

Where a point of attachment of

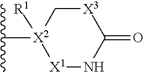

is depicted on Ring E, Ring F, or Ring G, it is intended, and one of ordinary skill in the art would appreciate, that the point of attachment of

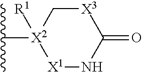

may be on any available carbon or nitrogen atom on Ring E, Ring F, or Ring G, including the carbon atom to which Ring E or Ring G arc fused to Ring F.

In some embodiments, a compound of formula I-e above is provided as a compound of formula I-e-1 or formula I-e-2:

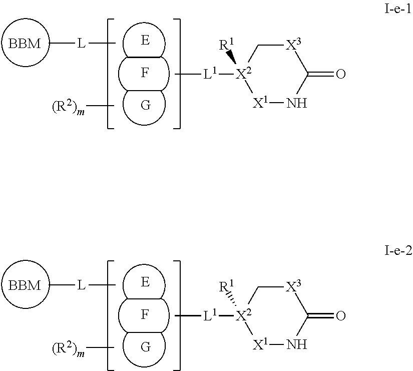

or a pharmaceutically acceptable salt thereof, wherein:
each of BBM, Ring E, Ring F, Ring G, L, L¹, R¹, R², X¹, X², X³, and m is as defined above.

In some embodiments, a compound of formula I-e above is provided as a compound of formula I-e-3:

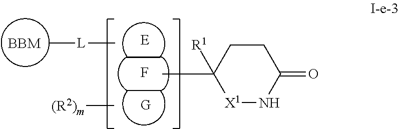

or a pharmaceutically acceptable salt thereof, wherein:
each of BBM, Ring E, Ring F, Ring G, L, R¹, R², X¹, and m is as defined above.

In certain embodiments, the present invention provides a compound of formula I, wherein LBM is an E3 ubiquitin ligase (cereblon) binding moiety thereby forming a compound of formula I-f:

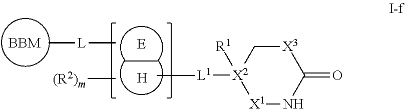

or a pharmaceutically acceptable salt thereof, wherein L and BBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

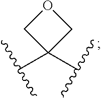 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring E is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
Ring H is a fused ring selected from a 7-9 membered saturated or partially unsaturated carbocyclyl or heterocyclyl ring with 1-3 heteroatoms independently selected from boron, nitrogen, oxygen, silicon, and sulfur, wherein Ring E is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3, or 4.

Where a point of attachment of

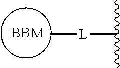

is depicted on Ring E or Ring H, it is intended, and one of ordinary skill in the art would appreciate, that the point of attachment of

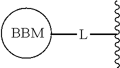

may be on any available carbon or nitrogen atom on Ring E or Ring H including the carbon atom to which Ring E and Ring H are fused.

Where a point of attachment of —(R²)ₘ is depicted on Ring E and Ring H, it is intended, and one of ordinary skill in the art would appreciate, that the point of attachment of —(R²)ₘ may be on any available carbon or nitrogen atom on Ring E or Ring H including the carbon atom to which Ring E and Ring H are fused.

Where a point of attachment of

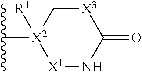

is depicted on Ring E and Ring H, it is intended, and one of ordinary skill in the art would appreciate, that the point of attachment of

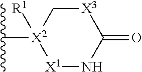

may be on any available carbon or nitrogen atom on Ring E or Ring H including the carbon atom to which Ring E and Ring H are fused.

In some embodiments, a compound of formula I-f above is provided as a compound of formula I-f-1 or formula I-f-2:

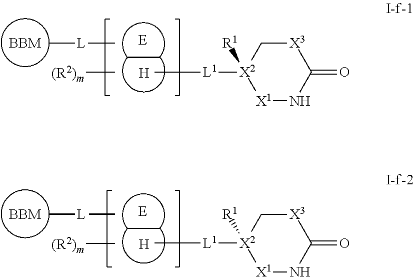

or a pharmaceutically acceptable salt thereof, wherein:
each of BBM, Ring E, Ring H, L, L¹, R¹, R², X¹, X², X³, and m is as defined above.

In some embodiments, a compound of formula I-f above is provided as a compound of formula I-f-3:

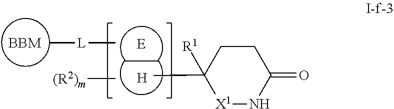

or a pharmaceutically acceptable salt thereof, wherein:
each of BBM, Ring E, Ring H, L, R¹, R², X¹, and m is as defined above.

In certain embodiments, the present invention provides a compound of formula I, wherein LBM is an E3 ubiquitin ligase (cereblon) binding moiety thereby forming a compound of formula I-g:

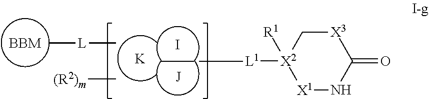

or a pharmaceutically acceptable salt thereof, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

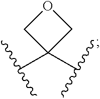 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —NR₂, —P(O)(OR)₂, —P(O)(NR₂) OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or;
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
   each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring I and J is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;
Ring K is a fused ring selected from a 5-12 membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—; and
m is 0, 1, 2, 3, or 4.

Where a point of attachment of

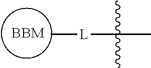

is depicted on Ring I, Ring J, and Ring K, it is intended, and one of ordinary skill in the art would appreciate, that the point of attachment of

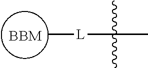

may be on any available carbon or nitrogen atom on Ring I, Ring J, or Ring K, including the carbon atom to which Ring I, Ring J, and Ring K are fused.

Where a point of attachment of —(R²)ₘ is depicted on Ring I, Ring J, and Ring K, it is intended, and one of ordinary skill in the art would appreciate, that the point of attachment of —(R²)ₘ may be on any available carbon or nitrogen atom on Ring I, Ring J, or Ring K, including the carbon atom to which Ring I, Ring J, and Ring K are fused.

Where a point of attachment of

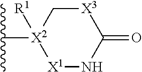

is depicted on Ring I, Ring J, and Ring K, it is intended, and one of ordinary skill in the art would appreciate, that the point of attachment of

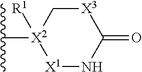

may be on any available carbon or nitrogen atom on Ring I, Ring J, or Ring K, including the carbon atom to which Ring I, Ring J, and Ring K are fused.

In some embodiments, a compound of formula I-g above is provided as a compound of formula I-g-1 or formula I-g-2:

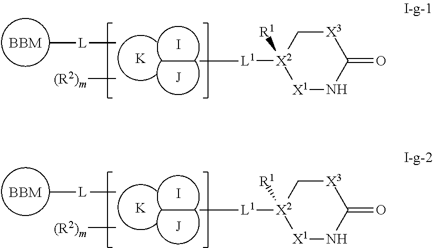

or a pharmaceutically acceptable salt thereof, wherein:
each of BBM, Ring I, Ring J, Ring K, L, L¹, R¹, R², X¹, X², X³, and m is as defined above.

In some embodiments, a compound of formula I-g above is provided as a compound of formula I-g-3:

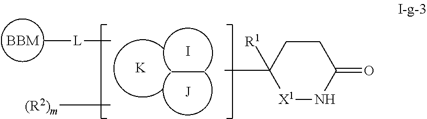

or a pharmaceutically acceptable salt thereof, wherein:
each of BBM, Ring I, Ring J, Ring K, L, R¹, R², X¹, and m is as defined above.

In certain embodiments, the present invention provides a compound of formula I, wherein LBM is an E3 ubiquitin ligase (cereblon) binding moiety thereby forming a compound of formula I-h-1 or I-h-2:

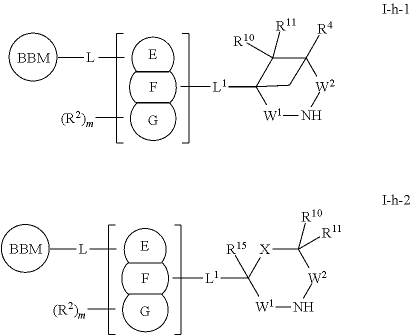

or a pharmaceutically acceptable salt thereof, wherein L and BBM are as defined above and described in embodiments herein, and wherein:
each R² is independently hydrogen, deuterium, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and Ring G is independently and optionally further substituted with 1-2 oxo groups;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3, 4, 5, 6, 7, 8, 9, 10, 11, 12, 13, 14, 15, or 16; and
R⁴, R¹⁰, R¹¹, R¹⁵, W¹, W², and X is as defined in WO 2019/099868, the entirety of each of which is herein incorporated by reference.

Where a point of attachment of

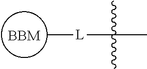

is depicted on Ring E, Ring F, or Ring G, it is intended, and one of ordinary skill in the art would appreciate, that the point of attachment of

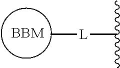

may be on any available carbon or nitrogen atom on Ring E, Ring F, or Ring G, including the ring to which Ring E or Ring G are fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G are fused to Ring F.

Where a point of attachment of

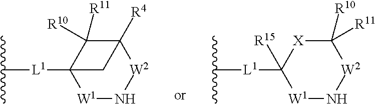

is depicted on Ring E, Ring F, or Ring G, it is intended, and one of ordinary skill in the art would appreciate, that the point of attachment of

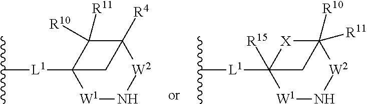

may be on any available carbon or nitrogen atom on Ring E, Ring F, or Ring G, including the carbon atom to which Ring E or Ring G are fused to Ring F.

As described above, in another aspect, the present invention provides a compound of formula I, wherein said compound is a compound of formula I-h-3:

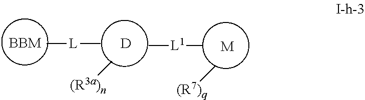

or a pharmaceutically acceptable salt thereof, wherein:
Ring M is selected from

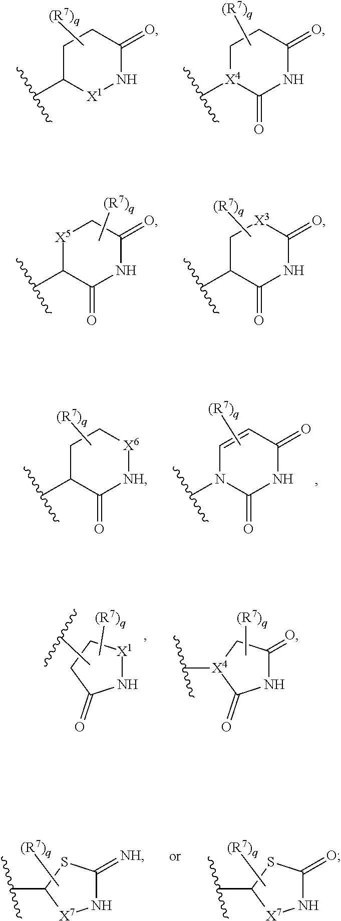

each of X¹, X⁶, and X⁷ is independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

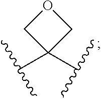 [SMILES: *C1(*)COC1]

each of X³ and X³ is independently a bivalent moiety selected from a covalent bond, —CR₂—, —NR—, —O—, —S—, or —SiR₂—;
X⁴ is a trivalent moiety selected from

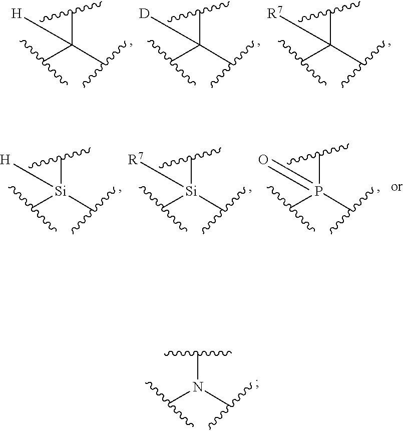

each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R^{3a} is independently hydrogen, deuterium, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁷ is independently hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic; or
   R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur;
   two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur;
   two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur, or a 7-13 membered saturated, partially unsaturated, bridged heterocyclic ring, or a spiro heterocyclic ring having 1-3 heteroatoms, independently selected from boron, nitrogen, oxygen, silicon, and sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CF(R)—, —C(F)₂—, —N(R)—, —S—, —S(O)₂— or —(C)═CH—;
n is 0, 1, 2, 3, or 4; and
q is 0, 1, 2, 3, or 4.

As defined above and described herein, each of X¹, X⁶, and X⁷ is independently a bivalent moiety selected from a covalent bond, —CH₂—, —C(R)₂—, —C(O)—, —C(S)—, —CH(R)—, —CH(CF₃)—, —P(O)(OR)—, —P(O)(R)—, —P(O)(NR₂)—, —S(O)—, —S(O)₂—, or

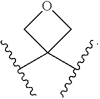 [SMILES: *C1(*)COC1]

In some embodiments, X¹, X⁶, and/or X⁷ is a covalent bond. In some embodiments, X¹, X⁶, and/or X⁷ is —CH₂—. In some embodiments, X¹, X⁶, and/or X⁷ is —CR₂—. In some embodiments, X¹, X⁶, and/or X⁷ is —C(O)—. In some embodiments, X¹, X⁶, and/or X⁷ is —C(S)—. In some embodiments, X¹, X⁶, and/or X⁷ is —CH(R)—. In some embodiments, X¹, X⁶, and/or X⁷ is —CH(CF₃)—. In some embodiments, X¹, X⁶, and/or X⁷ is —P(O)(OR)—. In some embodiments, X¹, X⁶, and/or X⁷ is —P(O)(R)—. In some embodiments, X¹, X⁶, and/or X⁷ is —P(O)NR₂—. In some embodiments, X¹, X⁶, and/or X⁷ is —S(O)—. In some embodiments, X¹, X⁶, and/or X⁷ is —S(O)₂—. In some embodiments, X¹, X⁶, and/or X⁷ is

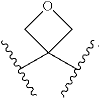 [SMILES: *C1(*)COC1]

In some embodiments, each of X¹, X⁶, and X⁷ are independently selected from those depicted in Table 1 below.

As defined above and described herein, X² is a carbon atom or silicon atom.

In some embodiments, X² is a carbon atom. In some embodiments, X² is a silicon atom.

In some embodiments, X² is selected from those depicted in Table 1, below.

As defined above and described herein, each of X³ and X⁵ is independently a bivalent moiety selected from —CH₂—, —CR₂—, —NR—, —CF₂—, —CHF—, —S—, —CH(R)—, —SiR₂—, or —O—.

In some embodiments, X³ and/or X⁵ is —CH₂—. In some embodiments, X³ and/or X⁵ is —CR₂—. In some embodiments, X³ and/or X⁵ is —NR—. In some embodiments, X³ and/or X⁵ is —CF₂—. In some embodiments, X³ and/or X⁵ is —CHF—. In some embodiments, X³ and/or X⁵ is —S—. In some embodiments, X³ and/or X⁵ is —CH(R)—. In some embodiments, X³ and/or X⁵ is —SiR₂—. In some embodiments, X³ and/or X⁵ is —O—.

In some embodiments, each of X³ and X⁵ is independently selected from those depicted in Table 1 below.

As defined above and described herein, X⁴ is a trivalent moiety selected from

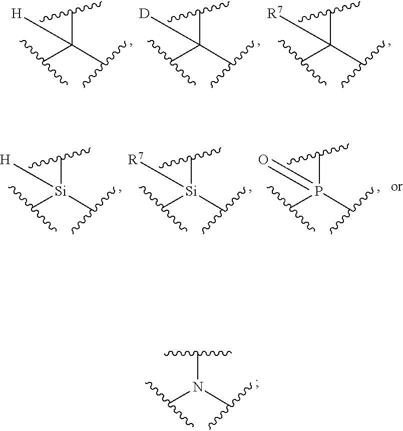

In some embodiments, X⁴ is

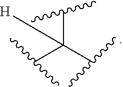 [SMILES: *C(*)*]

In some embodiments, X⁴ is

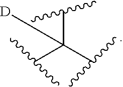 [SMILES: *C(*)(*)[2H]]

In some embodiments, X⁴ is

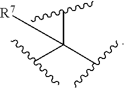

In some embodiments, X⁴ is

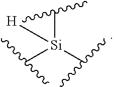 [SMILES: *[SiH](*)*]

In some embodiments, X⁴ is

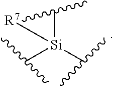

In some embodiments, X⁴ is

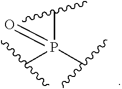 [SMILES: *P(*)(*)=O]

In some embodiments, X⁴ is

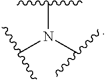 [SMILES: *N(*)*]

In some embodiments, X⁴ is selected from those depicted in Table 1 below.

As defined above and described herein, R¹ is hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, an optionally substituted C₁₋₄ aliphatic, or R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, and sulfur.

In some embodiments, R¹ is hydrogen. In some embodiments, R¹ is deuterium. In some embodiments, R¹ is halogen. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is —NR₂. In some embodiments, R¹ is —P(O)(OR)₂. In some embodiments, R¹ is —P(O)(NR₂)OR. In some embodiments, R¹ is —P(O)(NR₂)₂. In some embodiments, R¹ is —Si(OH)₂R. In some embodiments, R¹ is —Si(OH)(R)₂. In some embodiments, R¹ is —Si(R)₃. In some embodiments, R¹ is an optionally substituted C₁₋₄ aliphatic. In some embodiments, R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, and sulfur.

In some embodiments, R¹ is selected from those depicted in Table 1, below.

As defined above and described herein, each R is independently hydrogen, deuterium, or an optionally substituted group selected from C₁₋₆ aliphatic, phenyl, a 4-7 membered saturated or partially unsaturated heterocyclic having 1-3 heteroatoms independently selected from boron, nitrogen, oxygen, silicon, and sulfur, and a 5-6 membered heteroaryl ring having 1-4 heteroatoms independently selected from boron, nitrogen, oxygen, silicon, and sulfur, or two R groups on the same nitrogen are taken together with their intervening atoms to form a 4-7 membered saturated, partially unsaturated, or heteroaryl ring having 0-3 heteroatoms, in addition to the nitrogen, independently selected from boron, nitrogen, oxygen, silicon, and sulfur.

In some embodiments, R is hydrogen. In some embodiments, R is deuterium. In some embodiments, R is optionally substituted C₁₋₆ aliphatic. In some embodiments, R is optionally substituted phenyl. In some embodiments, R is optionally substituted 4-7 membered saturated or partially unsaturated heterocyclic having 1-3 heteroatoms independently selected from boron, nitrogen, oxygen, silicon, and sulfur. In some embodiments, R is optionally substituted 5-6 membered heteroaryl ring having 1-4 heteroatoms independently selected from boron, nitrogen, oxygen, silicon, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from boron, nitrogen, oxygen, silicon, and sulfur.

In some embodiments, R is selected from those depicted in Table 1, below.

As defined above and described herein, each of R² and R^{3a} is independently hydrogen, deuterium, —R⁶, halogen, —CN, —NO₂, —OR, —Si(OH)₃R, —Si(OH)R₂, —NR₂, —SiR₃, —S(O)₂R, —S(O)₃NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R.

In some embodiments, R² and/or R^{3a} is hydrogen. In some embodiments, R² and/or R^{3a} is deuterium. In some embodiments, R² and/or R^{3a} is —R⁶. In some embodiments, R² and/or R^{3a} is halogen. In some embodiments, R² and/or R^{3a} is —CN. In some embodiments, R² and/or R^{3a} is —NO₂. In some embodiments, R² and/or R^{3a} is —OR. In some embodiments, R² and/or R^{3a} is —Si(OH)₃R. In some embodiments, R² and/or R^{3a} is —Si(OH)R₂. In some embodiments, R² and/or R^{3a} is —SR. In some embodiments, R² and/or R^{3a} is —NR₂. In some embodiments, R² and/or R^{3a} is —SiR₃. In some embodiments, R² and/or R^{3a} is —S(O)₂R. In some embodiments, R² and/or R^{3a} is —S(O)₂NR₂. In some embodiments, R² and/or R^{3a} is —S(O)R. In some embodiments, R² and/or R^{3a} is —C(O)R. In some embodiments, R² and/or R^{3a} is —C(O)OR. In some embodiments, R² and/or R^{3a} is —C(O)NR₂. In some embodiments, R² and/or R^{3a} is —C(O)N(R)OR. In some embodiments, R² and/or R^{3a} is —C(R)₃N(R)C(O)R. In some embodiments, R² and/or R^{3a} is —C(R)₂N(R)C(O)NR₂. In some embodiments, R² and/or R^{3a} is —OC(O)R. In some embodiments, R² and/or R^{3a} is —OC(O)NR₂. In some embodiments, R² and/or R^{3a} is —OP(O)R₂. In some embodiments, R² and/or R^{3a} is —OP(O)(OR)₂. In some embodiments, R² and/or R^{3a} is —OP(O)(OR)NR₂. In some embodiments, R² and/or R^{3a} is —OP(O)(NR₂)₂—. In some embodiments, R² and/or R^{3a} is —N(R)C(O)OR. In some embodiments, R² and R^{3a} is independently —N(R)C(O)R. In some embodiments, R² and/or R^{3a} is —N(R)C(O)NR₂. In some embodiments, R² and/or R^{3a} is —NP(O)R₂. In some embodiments, R² and/or R^{3a} is —N(R)P(O)(OR)₂. In some embodiments, R² and/or R^{3a} is —N(R)P(O)(OR)NR₂. In some embodiments, R² and/or R^{3a} is —N(R)P(O)(NR₂)₂. In some embodiments, R² and/or R^{3a} is —N(R)S(O)₂R.

In some embodiments, R² and R^{3a} is independently —OH. In some embodiments, R² and R^{3a} is independently —NH₂. In some embodiments, R² and R^{3a} is independently —CH₂NH₂. In some embodiments, R² and R^{3a} is independently —CH₂NHCOMe. In some embodiments, R² and R^{3a} is independently —CH₂NHCONHMe. In some embodiments, R² and R^{3a} is independently —NHCOMe. In some embodiments, R² and R^{3a} is independently —NHCONHEt. In some embodiments, R² and R^{3a} is independently —SiMe₂. In some embodiments, R² and R^{3a} is independently —SiMe₂OH. In some embodiments, R² and R^{3a} is independently —SiMe(OH)₂. In some embodiments R² and/or R^{3a} is

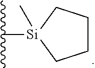 [SMILES: *[Si]1(C)CCCC1]

In some embodiments, R² and/or R^{3a} is Br. In some embodiments, R² and/or R^{3a} is Cl. In some embodiments, R² and/or R^{3a} is F. In some embodiments, R² and/or R^{3a} is Me. In some embodiments, R² and/or R^{3a} is —NHMe. In some embodiments, R² and/or R^{3a} is —NMe₂. In some embodiments, R² and/or R^{3a} is —NHCO₂Et. In some embodiments, R² and/or R^{3a} is —CN. In some embodiments, R² and/or R^{3a} is —CH₂Ph. In some embodiments, R² and/or R^{3a} is —NHCO₂tBu. In some embodiments, R² and/or R^{3a} is —CO₂tBu. In some embodiments, R² and/or R^{3a} is —OMe. In some embodiments, R² and/or R^{3a} is —CF₃.

In some embodiments, R² and R^{3a} are selected from those depicted in Table 1, below.

As defined above and described herein, R³ is hydrogen, deuterium, halogen, CN, NO₂, —OR, —NR₂, —SR, —S(O)₂R, —S(O)₂NR₂, —S(O)R, —C(O)R, —C(O)OR, —C(O)NR₂, —C(O)NR(OR), —OC(O)R, —OC(O)NR₂, —OP(O)(OR)₂, —OP(O)(NR₂)₂, —OP(O)(OR)NR₂, —N(R)C(O)R, —N(R)C(O)OR, —N(R)C(O)NR₂, —N(R)S(O)₂R, —N(R)S(O)₂NR₂, —N(R)P(O)(OR)₂, —N(R)P(O)(OR)NR₂, —P(O)(OR)₂, —P(O)(NR₂)OR, —P(O)(NR₂)₂, —Si(OH)₂R, —Si(OH)(R)₂, or —Si(R)₃.

In some embodiments, R³ is hydrogen. In some embodiments, R³ is deuterium. In some embodiments, R₃ is halogen. In some embodiments, R³ is CN. In some embodiments, R³ is —NO₂. In some embodiments, R³ is —OR. In some embodiments, R³ is —NR₂. In some embodiments, R³ is —SR. In some embodiments, R³ is —S(O)₂R. In some embodiments, R³ is —S(O)₂NR₂. In some embodiments, R³ is —S(O)R. In some embodiments, R³ is —C(O)R. In some embodiments, R³ is —C(O)OR. In some embodiments, R³ is —C(O)NR₂. In some embodiments, R³ is —C(O)NR(OR). In some embodiments, R³ is —OC(O)R. In some embodiments, R³ is —OC(O)NR₂. In some embodiments, R³ is —OP(O)(OR)₂. In some embodiments, R³ is —OP(O)(NR₂)₂. In some embodiments, R³ is —OP(O)(OR)NR₂. In some embodiments, R³ is —N(R)C(O)R. In some embodiments, R³ is —N(R)C(O)OR. In some embodiments, R³ is —N(R)C(O)NR₂. In some embodiments, R³ is —N(R)S(O)₂R. In some embodiments, R³ is —N(R)S(O)₂NR₂. In some embodiments, R³ is —N(R)P(O)(OR)₂. In some embodiments, R³ is —N(R)P(O)(OR)NR₂. In some embodiments, R³ is —P(O)(OR)₂. In some embodiments, R³ is —P(O)(NR₂)OR. In some embodiments, R³ is —P(O)(NR₂)₂. In some embodiments, R³ is —Si(OH)₂R. In some embodiments, R³ is —Si(OH)(R)₂. In some embodiments, R³ is —Si(R)₃.

In some embodiments, R³ is methyl. In some embodiments, R³ is —OCH₃. In some embodiments, R³ is chloro.

In some embodiments, R³ is selected from those depicted in Table 1, below.

As defined above and described herein, each R⁴ is independently hydrogen, deuterium, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —P(O)(OR)₂, —P(O)(NR₂)OR, or —P(O)(NR₂)₂.

In some embodiments, R⁴ is hydrogen. In some embodiments, R⁴ is —R⁶. In some embodiments, R⁴ is halogen. In some embodiments, R⁴ is —CN. In some embodiments, R⁴ is —NO₂. In some embodiments, R⁴ is OR. In some embodiments, R⁴ is SR. In some embodiments, R⁴ is —NR₂. In some embodiments, R⁴ is —S(O)₂R. In some embodiments, R⁴ is —S(O)₂NR₂. In some embodiments, R⁴ is —S(O)R. In some embodiments, R⁴ is —C(O)R. In some embodiments, R⁴ is —C(O)OR. In some embodiments, R⁴ is —C(O)NR₂. In some embodiments, R⁴ is —C(O)N(R)OR. In some embodiments, R⁴ is —OC(O)R. In some embodiments, R⁴ is —OC(O)NR₂. In some embodiments, R⁴ is —N(R)C(O)OR. In some embodiments, R⁴ is —N(R)C(O)R. In some embodiments, R⁴ is —N(R)C(O)NR₂. In some embodiments, R⁴ is —N(R)S(O)₂R. In some embodiments, R⁴ is —P(O)(OR)₂. In some embodiments, R⁴ is —P(O)(NR₂) OR. In some embodiments, R⁴ is —P(O)(NR₂)₂.

In some embodiments, R⁴ is methyl. In some embodiments, R⁴ is ethyl. In some embodiments, R⁴ is cyclopropyl.

In some embodiments, R⁴ is selected from those depicted in Table 1, below.

As defined above and described herein, R⁵ is hydrogen, deuterium, an optionally substitute C₁₋₄ aliphatic, or —CN.

In some embodiments, R⁵ is hydrogen. In some embodiments, R⁵ is deuterium. In some embodiments, R⁵ is an optionally substituted C₁₋₄ aliphatic. In some embodiments, R⁵ is —CN.

In some embodiments, R⁵ is selected from those depicted in Table 1, below.

As defined above and described herein, each R⁶ is independently an optionally substituted group selected from C₁₋₆ aliphatic, phenyl, a 4-7 membered saturated or partially unsaturated heterocyclic ring having 1-3 heteroatoms independently selected from boron, nitrogen, oxygen, silicon, and sulfur, and a 5-6 membered heteroaryl ring having 1-4 heteroatoms independently selected from boron, nitrogen, oxygen, silicon, and sulfur.

In some embodiments, R⁶ is an optionally substituted C₁₋₆ aliphatic. In some embodiments, R⁶ is an optionally substituted phenyl. In some embodiments, R⁶ is an optionally substituted 4-7 membered saturated or partially unsaturated heterocyclic ring having 1-3 heteroatoms independently selected from boron, nitrogen, oxygen, silicon, and sulfur. In some embodiments, R⁶ is an optionally substituted 5-6 membered heteroaryl ring having 1-4 heteroatoms independently selected from boron, nitrogen, oxygen, silicon, and sulfur.

In some embodiments, R⁶ is selected from those depicted in Table 1, below.

As defined generally above, each R⁷ is independently hydrogen, deuterium, halogen, —CN, —OR, —SR, —S(O)R, —S(O)₂R, —N(R)₂, —P(O)(R)₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic, or R¹ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur, or two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur, or two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur, or a 7-13 membered saturated, partially unsaturated, bridged heterocyclic ring, or a spiro heterocyclic ring having 1-3 heteroatoms, independently selected from boron, nitrogen, oxygen, silicon, and sulfur.

In some embodiments, R⁷ is hydrogen. In some embodiments, R⁷ is deuterium. In some embodiments, R⁷ is halogen. In some embodiments, R⁷ is —CN. In some embodiments, R⁷ is —OR. In some embodiments, R⁷ is —SR. In some embodiments, R⁷ is —S(O)R. In some embodiments, R⁷ is —S(O)₂R. In some embodiments, R⁷ is —NR₂. In some embodiments, R⁷ is —Si(R)₃. In some embodiments, R⁷ is —P(O)(R)₂. In some embodiments, R⁷ is —P(O)(OR)₂. In some embodiments, R⁷ is —P(O)(NR₂)OR. In some embodiments, R⁷ is —P(O)(NR₂)₂. In some embodiments, R⁷ is —Si(OH)R₂. In some embodiments, R⁷ is —Si(OH)₂R. In some embodiments, R⁷ is an optionally substituted C₁₋₄ aliphatic. In some embodiments, R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, and sulfur. In some embodiments, two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, and sulfur. In some embodiments, two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, and sulfur. In some embodiments, two R⁷ groups on adjacent carbon atoms are optionally taken together with their intervening atoms to form a 7-13 membered saturated, partially unsaturated, bridged heterocyclic ring, or a spiro heterocyclic ring having 1-3 heteroatoms, independently selected from boron, nitrogen, oxygen, silicon, and sulfur.

In some embodiments, R⁷ is selected from hydrogen, halogen, —CN, —OR, —NR₂, or C₁₋₄ alkyl. In some embodiments, R⁷ is selected from hydrogen, halogen, —CN, or C₁₋₄ alkyl. In some embodiments, R⁷ is fluoro. In some embodiments, two R⁷ groups on the same carbon are optionally taken together with their intervening atoms to form a 3- or 4-membered spiro fused ring.

In some embodiments, R⁷ is selected from those depicted in Table 1 below.

As defined above and described herein, Ring A is a bi- or tricyclic ring selected from

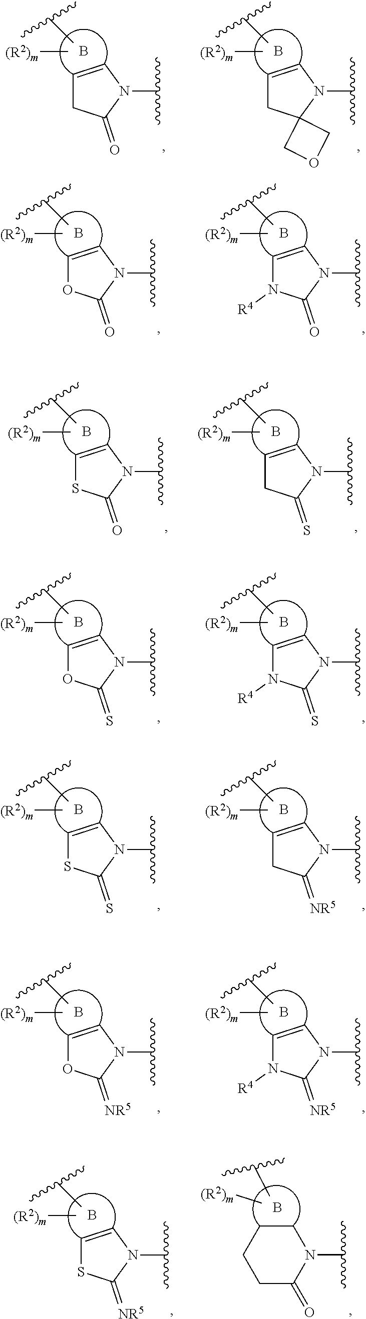

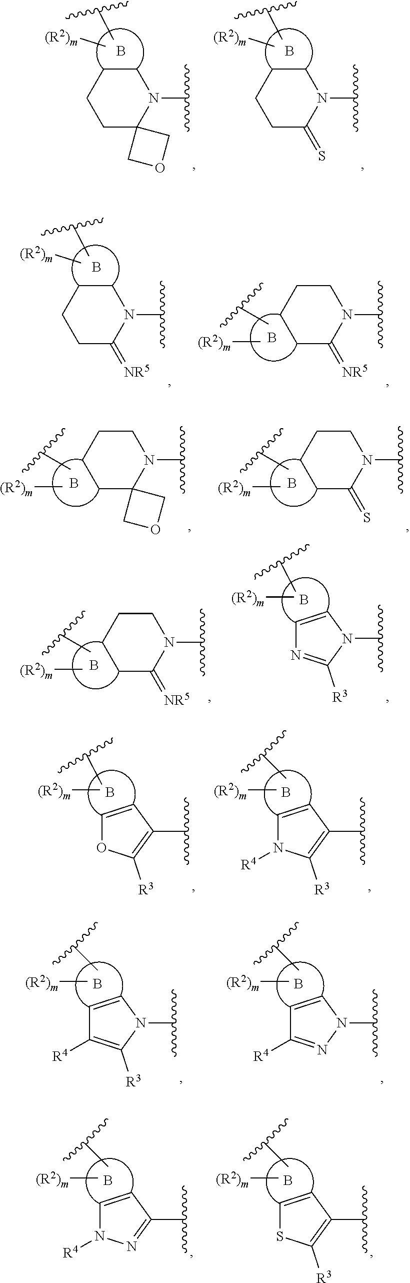

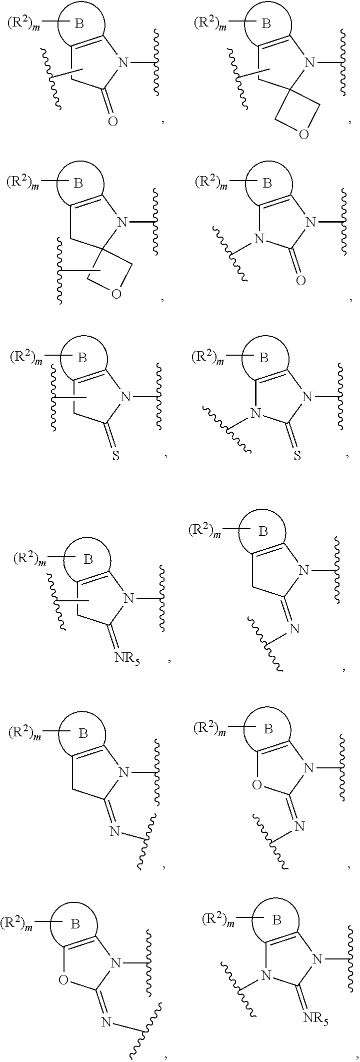

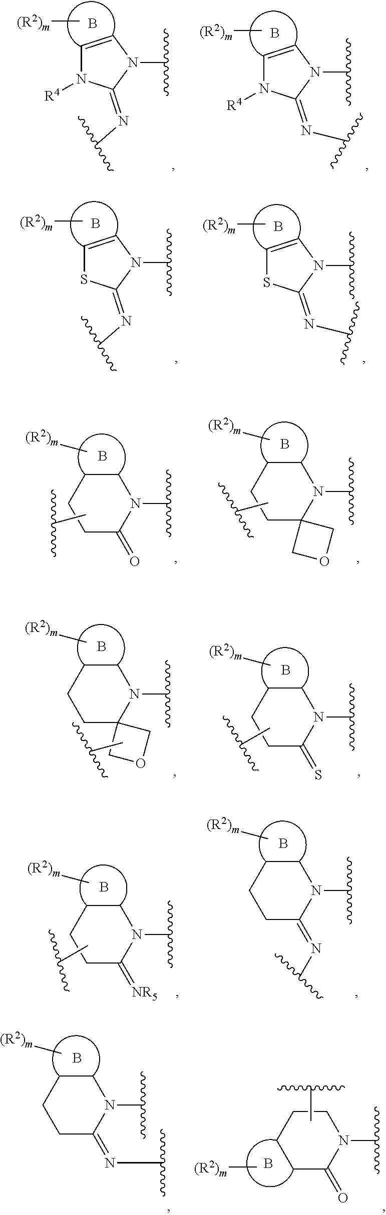

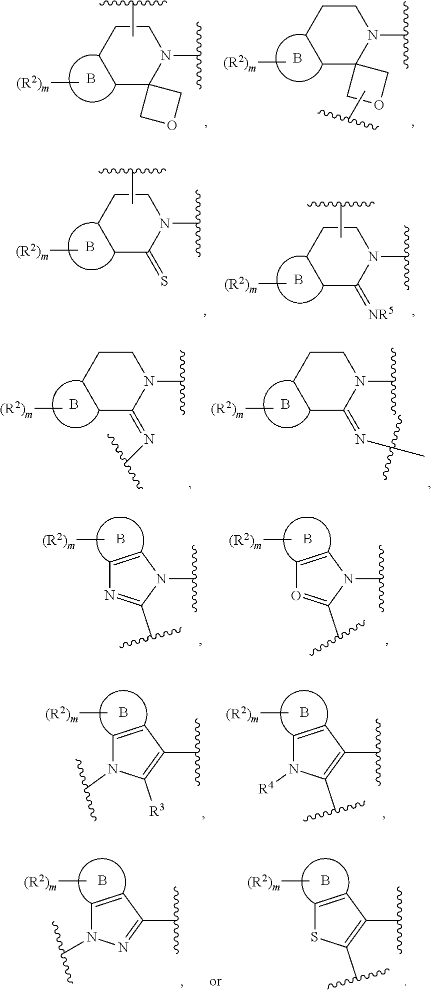

In some embodiments, Ring A is

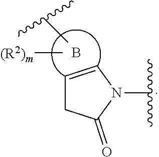

In some embodiments, Ring A is

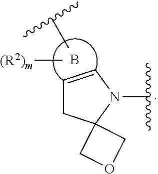

In some embodiments, Ring A is

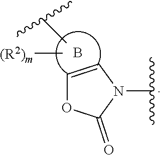

In some embodiments, Ring A is

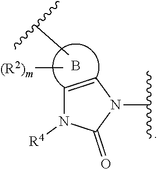

In some embodiments, Ring A is

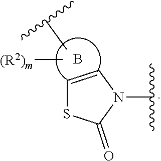

In some embodiments, Ring A is

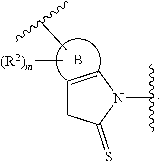

In some embodiments, Ring A is

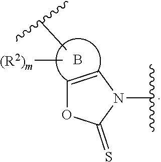

In some embodiments, Ring A is

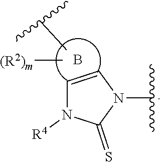

In some embodiments, Ring A is

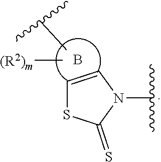

In some embodiments, Ring A is

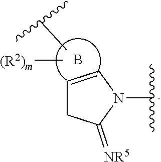

In some embodiments, Ring A is

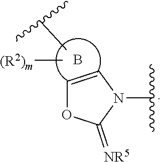

In some embodiments, Ring A is

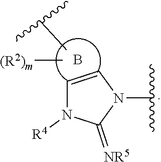

In some embodiments, Ring A is

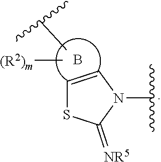

In some embodiments, Ring A is

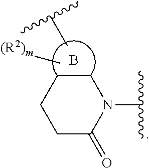

In some embodiments, Ring A is

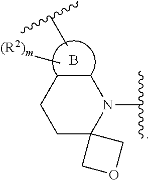

In some embodiments, Ring A is

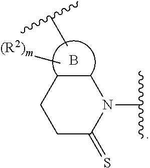

In some embodiments, Ring A is

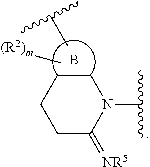

In some embodiments, Ring A is

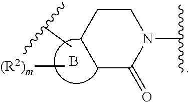

In some embodiments, Ring A is

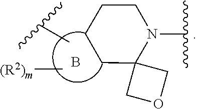

In some embodiments, Ring A is

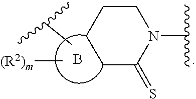

In some embodiments, Ring A is

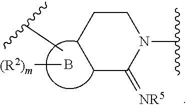

In some embodiments, Ring A is

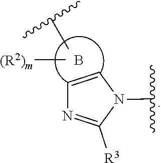

In some embodiments, Ring A is

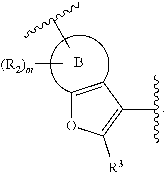

In some embodiments, Ring A is

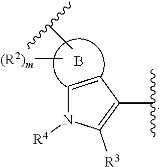

In some embodiments, Ring A is

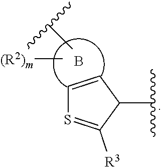

In some embodiments, Ring A is

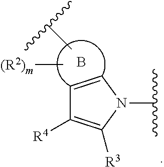

In some embodiments, Ring A is

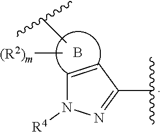

In some embodiments, Ring A is

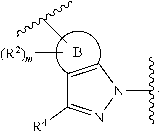

In some embodiments, Ring A is

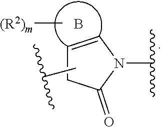

In some embodiments, Ring A is

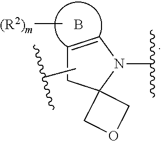

In some embodiments, Ring A is

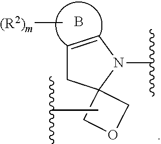

In some embodiments, Ring A is

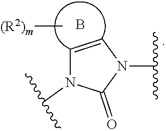

In some embodiments, Ring A is

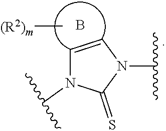

In some embodiments, Ring A is

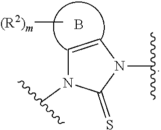

In some embodiments, Ring A is

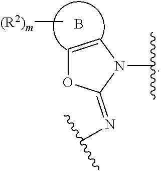

In some embodiments, Ring A is

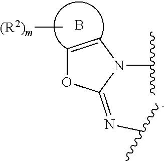

In some embodiments, Ring A is

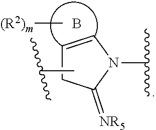

In some embodiments, Ring A is

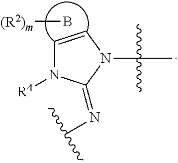

In some embodiments, Ring A is

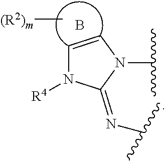

In some embodiments, Ring A is

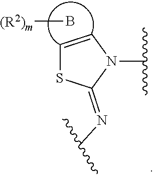

In some embodiments, Ring A is

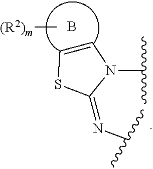

In some embodiments, Ring A is

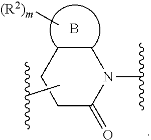

In some embodiments, Ring A is

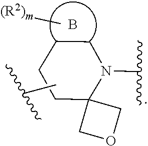

In some embodiments, Ring A is

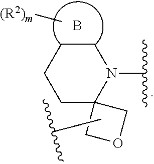

In some embodiments, Ring A is

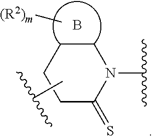

In some embodiments, Ring A is

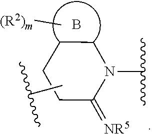

In some embodiments, Ring A is

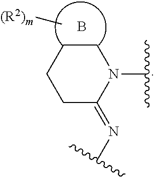

In some embodiments, Ring A is

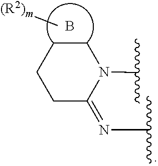

In some embodiments, Ring A is

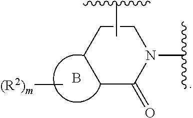

In some embodiments, Ring A is

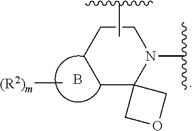

In some embodiments, Ring A is

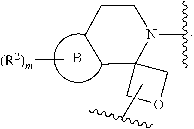

In some embodiments, Ring A is

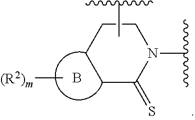

In some embodiments, Ring A is

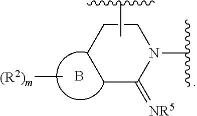

In some embodiments, Ring A is

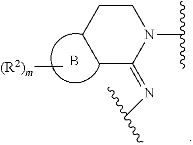

In some embodiments, Ring A is

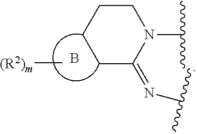

In some embodiments, Ring A is

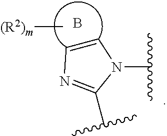

In some embodiments, Ring A is

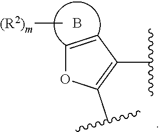

In some embodiments, Ring A is

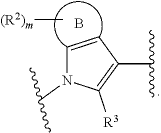

In some embodiments, Ring A is

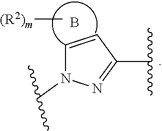

In some embodiments, Ring A is

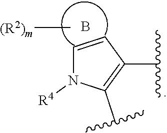

In some embodiments, Ring A is

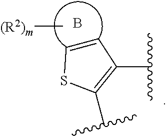

In some embodiments, Ring A is selected from those depicted in Table 1, below.

As defined above and described herein, Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur;

In some embodiments, Ring B is a fused 6-membered aryl. In some embodiments, Ring B is a fused 6-membered heteroaryl containing 1-4 heteroatoms independently selected from nitrogen, oxygen, and sulfur. In some embodiments, Ring B is a fused 5 to 7-membered saturated or partially unsaturated carbocyclyl. In some embodiments, Ring B is fused 5 to 7-membered saturated or partially saturated heterocyclyl with 1-3 heteroatoms independently selected from boron, nitrogen, oxygen, silicon, and sulfur. In some embodiments, Ring B is fused 5-membered heteroaryl with 1-4 heteroatoms independently selected from boron, nitrogen, oxygen, silicon, and sulfur.

In some embodiments, Ring B is

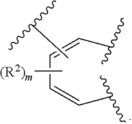

In some embodiments, Ring B is

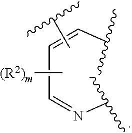

In some embodiments, Ring B is

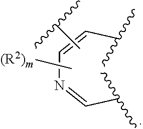

In some embodiments, Ring B is selected from those depicted in Table 1, below.

As defined above and described herein, Ring C is a mono- or bicyclic ring selected from

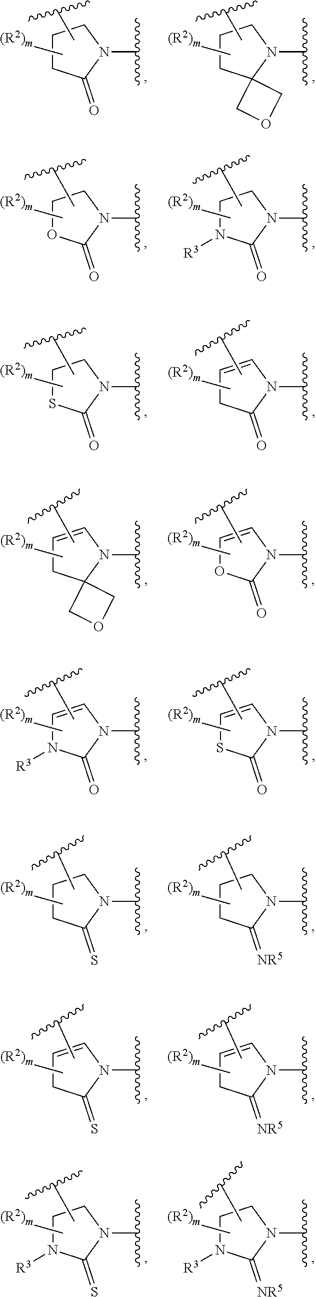

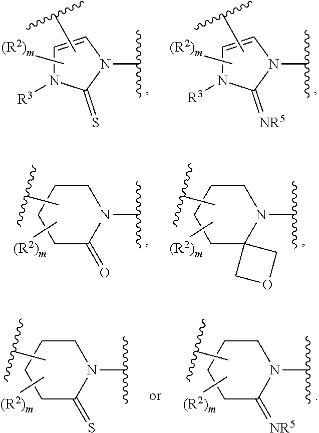

In some embodiments, Ring A and Ring B is

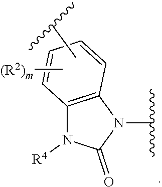

In some embodiments, Ring A and Ring B is

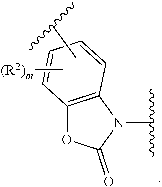

In some embodiments, Ring A and Ring B is

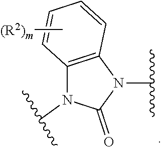

In some embodiments, Ring A and Ring B is

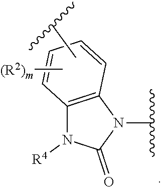

In some embodiments, Ring C is

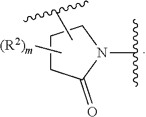

In some embodiments, Ring C is

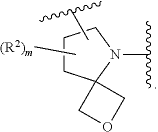

In some embodiments, Ring C is

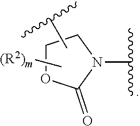

In some embodiments, Ring C is

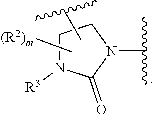

In some embodiments, Ring C is

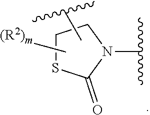

In some embodiments, Ring C is

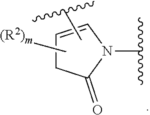

In some embodiments, Ring C is

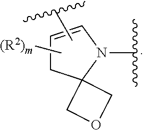

In some embodiments, Ring C is

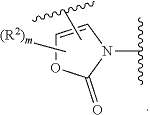

In some embodiments, Ring C is

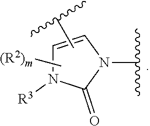

In some embodiments, Ring C is

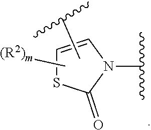

In some embodiments, Ring C is

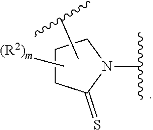

In some embodiments, Ring C is

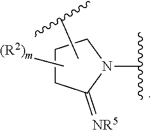

In some embodiments, Ring C is

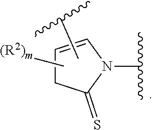

In some embodiments, Ring C is

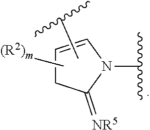

In some embodiments, Ring C is

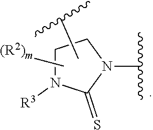

In some embodiments, Ring C is

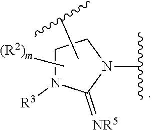

In some embodiments, Ring C is

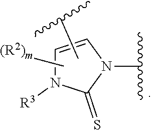

In some embodiments, Ring C is

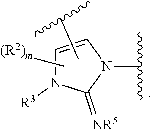

In some embodiments, Ring C is

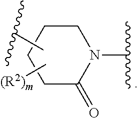

In some embodiments, Ring C is

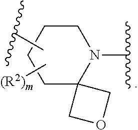

In some embodiments, Ring C is

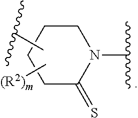

In some embodiments, Ring C is

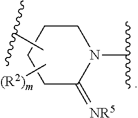

In some embodiments, Ring C is

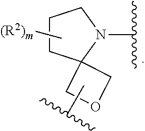

In some embodiments, Ring C is

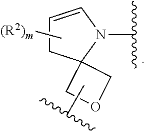

In some embodiments, Ring C is

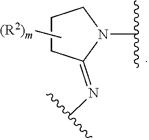

In some embodiments, Ring C is

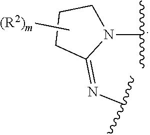

In some embodiments, Ring C is

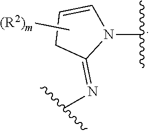

In some embodiments, Ring C is

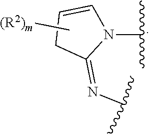

In some embodiments, Ring C is

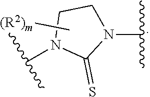

In some embodiments, Ring C is

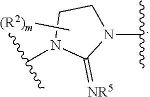

In some embodiments, Ring C is

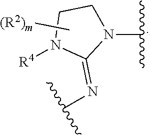

In some embodiments, Ring C is

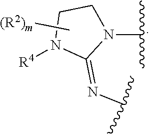

In some embodiments, Ring C is

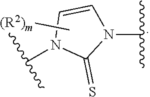

In some embodiments, Ring C is

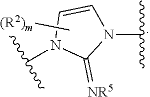

In some embodiments, Ring C is

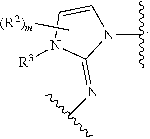

In some embodiments, Ring C is

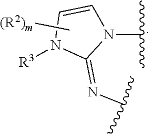

In some embodiments, Ring C is

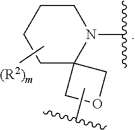

In some embodiments, Ring C is

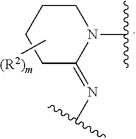

In some embodiments, Ring C is

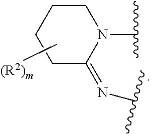

In some embodiments, Ring C is a mono- or bicyclic ring selected from

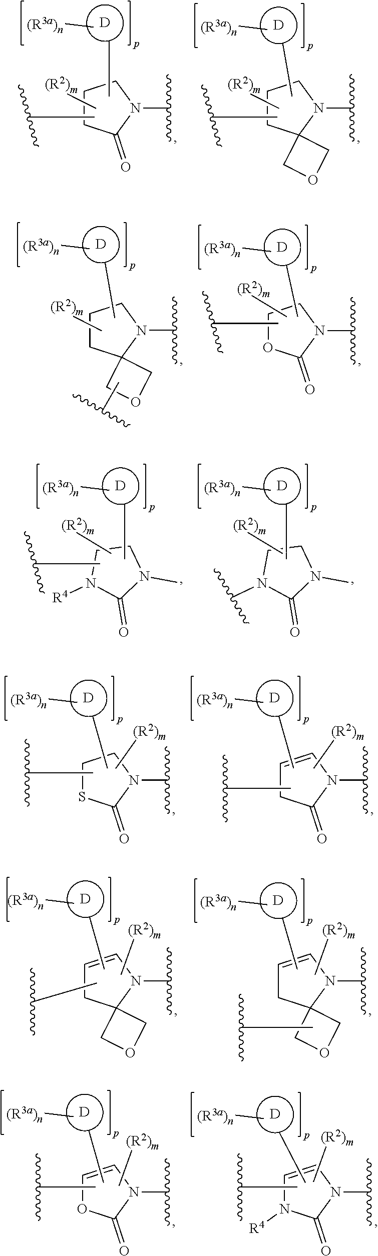

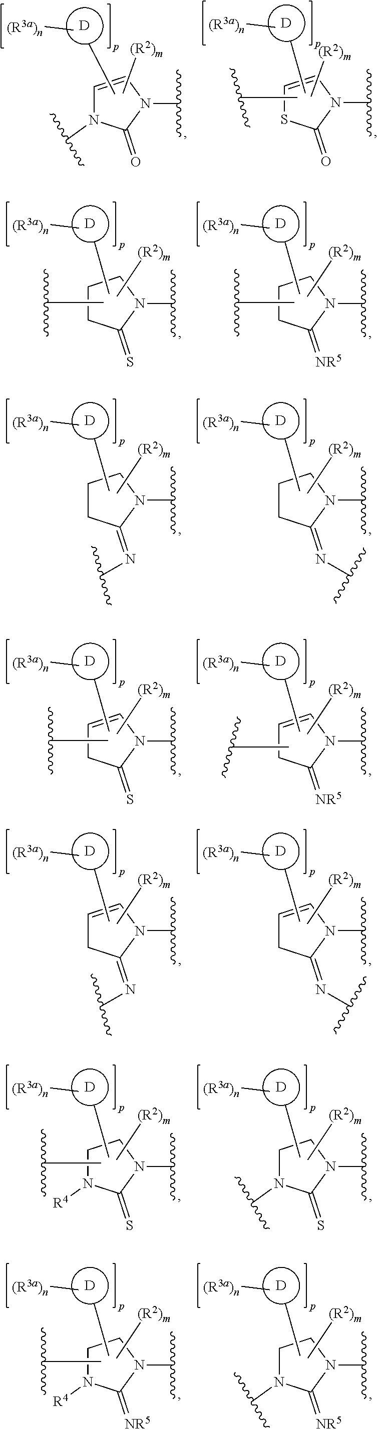

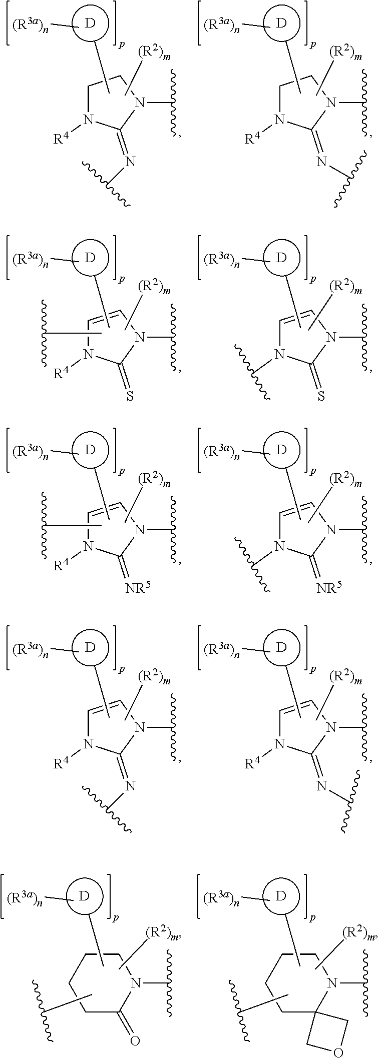

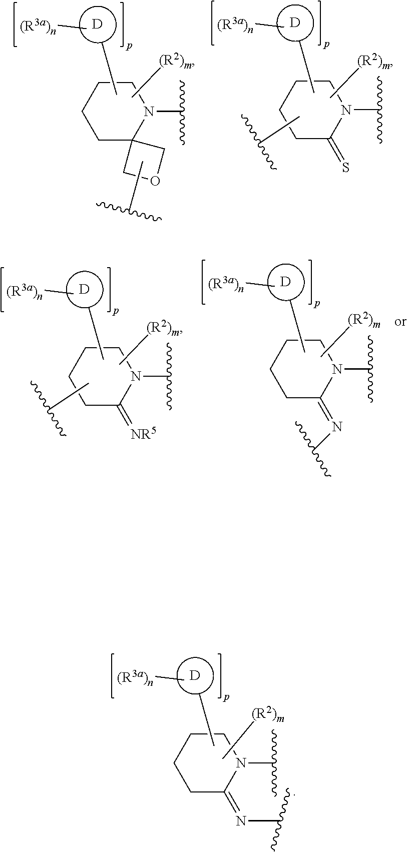

In some embodiments, Ring C is selected from

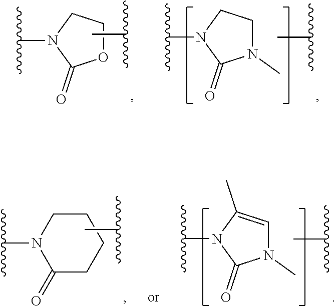

In some embodiments, Ring C is selected from

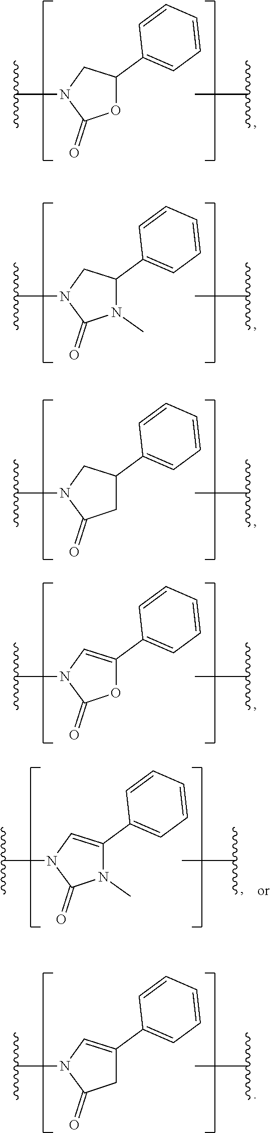

In some embodiments, Ring C is selected from those depicted in Table 1, below.

As defined above and described herein, Ring D is a ring selected from a 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;

In some embodiments, Ring D is a 6 to 10-membered aryl. In some embodiments, Ring D is a 6 to 10-membered heteroaryl containing 1-4 heteroatoms independently selected from nitrogen, oxygen, and sulfur. In some embodiments, Ring D is a 5 to 7-membered saturated or partially unsaturated carbocyclyl. In some embodiments, Ring D is 5 to 7-membered saturated or partially saturated heterocyclyl with 1-3 heteroatoms independently selected from boron, nitrogen, oxygen, silicon, and sulfur. In some embodiments, Ring D is 5-membered heteroaryl with 1-4 heteroatoms independently selected from boron, nitrogen, oxygen, silicon, and sulfur.

In some embodiments, Ring D is indazole. In some embodiments, Ring D is quinoline. In some embodiments, Ring D is isoquinoline. In some embodiments, Ring D is imidazo[1,2-a]pyridine.

In some embodiments, Ring D is selected from those depicted in Table 1 below.

As defined above and described herein, each of Ring E, Ring F, and Ring G is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur, wherein each of Ring E, Ring F, and Ring G is independently and optionally further substituted with 1-2 oxo groups.

In some embodiments, one or more of Ring E, Ring F, and Ring G is a 6-membered aryl. In some embodiments, one or more of Ring E, Ring F, and Ring G is a 6-membered heteroaryl containing 1-4 heteroatoms independently selected from nitrogen, oxygen, and sulfur. In some embodiments, one or more of Ring E, Ring F, and Ring G is a 5 to 7-membered saturated or partially unsaturated carbocyclyl. In some embodiments, one or more of Ring E, Ring F, and Ring G is a 5 to 7-membered saturated or partially unsaturated heterocyclyl with 1-3 heteroatoms independently selected from boron, nitrogen, oxygen, silicon, and sulfur. In some embodiments, one or more of Ring E, Ring F, and Ring G is a 5-membered heteroaryl with 1-4 heteroatoms independently selected from nitrogen, oxygen and sulfur. In some embodiments, one or more of Ring E, Ring F, and Ring G is and optionally further substituted with 1-2 oxo groups.

In some embodiments, Ring E, Ring F, and Ring G are selected from those depicted in Table 1, below.

As defined above and described herein, Ring H is a ring selected from a 7-9 membered saturated or partially unsaturated carbocyclyl or heterocyclyl ring with 1-3 heteroatoms independently selected from boron, nitrogen, oxygen, silicon, and sulfur, wherein Ring E is optionally further substituted with 1-2 oxo groups.

In some embodiments, Ring H is a ring selected from a 7-9 membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups.

In some embodiments, Ring E and Ring H is selected from those depicted in Table 1, below.

As defined above and described herein, each of Ring I and Ring J is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen and sulfur

In some embodiments, each of Ring I and Ring J is independently a 6-membered aryl. In some embodiments, each of Ring I and Ring J is independently a 6-membered heteroaryl containing 1-4 heteroatoms independently selected from nitrogen, oxygen, and sulfur. In some embodiments, each of Ring I and Ring J is independently a 5 to 7-membered saturated or partially unsaturated carbocyclyl. In some embodiments, each of Ring I and Ring J is independently a 5 to 7-membered saturated or partially unsaturated heterocyclyl ring with 1-3 heteroatoms independently selected from boron, nitrogen, oxygen, silicon, and sulfur. In some embodiments, each of Ring I and Ring J is independently a 5-membered heteroaryl with 1-3 heteroatoms independently selected from nitrogen, oxygen and sulfur.

As defined above and described herein, Ring K is a fused ring selected from a 6-12 membered saturated or partially unsaturated carbocyclyl or heterocyclyl ring with 1-3 heteroatoms independently selected from boron, nitrogen, oxygen, silicon, and sulfur, wherein Ring H is optionally further substituted with 1-2 oxo groups.

In some embodiments, Ring K is a fused ring selected from a 6-12 membered saturated or partially unsaturated carbocyclyl. In some embodiments, Ring K is a 6-12 membered saturated or partially unsaturated heterocyclyl ring with 1-3 heteroatoms independently selected from boron, nitrogen, oxygen, silicon, and sulfur. In some embodiments, Ring K is optionally further substituted with 1-2 oxo groups.

In some embodiments, Ring I, Ring J, and Ring K is selected from those depicted in Table 1, below.

As defined above and described herein, Ring M is selected from

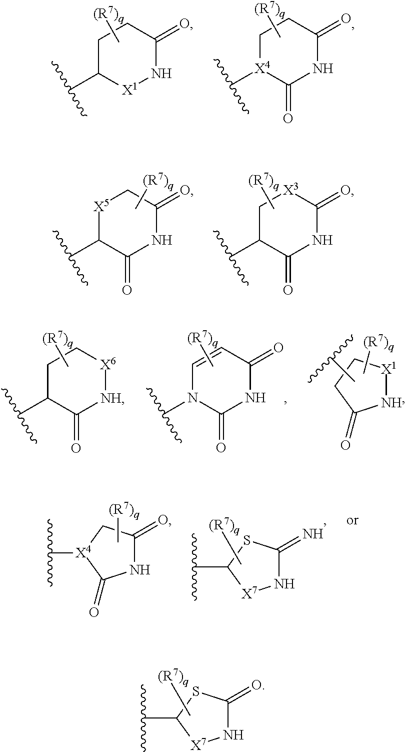

In some embodiments, Ring M is

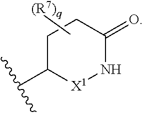

In some embodiments, Ring M is

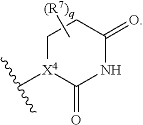

In some embodiments, Ring M is

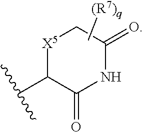

In some embodiments, Ring M is

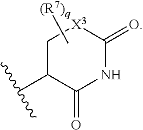

In some embodiments, Ring M is

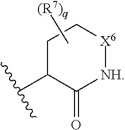

In some embodiments, Ring M is

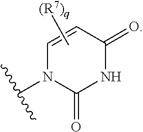

In some embodiments, Ring M is

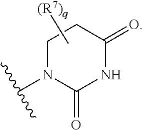

In some embodiments, Ring M is

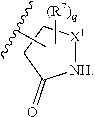

In some embodiments, Ring M is

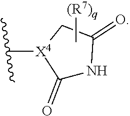

In some embodiments, Ring M is

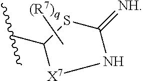

In some embodiments, Ring M is

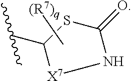

In some embodiments, Ring M is selected from those depicted in Table 1 below.

As defined above and described here, L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;

In some embodiments, L¹ is a covalent bond. In some embodiments, L¹ is a C₁₋₃ aliphatic. In some embodiments, L¹ is —CH₂—. In some embodiments, L¹ is —C(D)(H)—. In some embodiments, L¹ is —C(D)₂-. In some embodiments, L¹ is —CH₂CH₂—. In some embodiments, L¹ is —NR—. In some embodiments, L¹ is —CH₂NR—. In some embodiments, L¹ is or —O—. In some embodiments, L¹ is —CH₂O—. In some embodiments, L¹ is —S—. In some embodiments, L¹ is —OC(O)—. In some embodiments, L¹ is —C(O)O—. In some embodiments, L¹ is —C(O)—. In some embodiments, L¹ is —S(O)—. In some embodiments, L¹ is —S(O)₂—. In some embodiments, L¹ is —NRS(O)₂—. In some embodiments, L¹ is —S(O)₂NR—. In some embodiments, L¹ is —NRC(O)—. In some embodiments, L¹ is —C(O)NR—.

In some embodiments, Ring L¹ is selected from those depicted in Table 1, below.

As defined above and described herein, [symbol] is a single or double bond.

In some embodiments, [symbol] is a single bond. In some embodiments, [symbol] is a double bond.

In some embodiments, [symbol] is selected from those depicted in Table 1, below.

As defined above and described herein, m is 0, 1, 2, 3, 4, 5, 6, 7, 8, 9, 10, 11, 12, 13, 14, 15, or 16.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16.

In some embodiments, m is selected from those depicted in Table 1, below.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In some embodiments, n is selected from those depicted in Table 1, below.

As defined above and described herein, p is 0 or 1.

In some embodiments, p is 0. In some embodiments, p is 1.

In some embodiments, p is selected from those depicted in Table 1, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1 below.

In some embodiments, LBM is

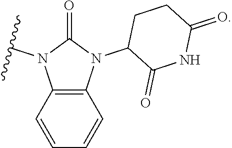 [SMILES: *n1c(=O)n(C2CCC(=O)NC2=O)c2ccccc21]

In some embodiments, LBM is

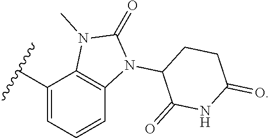 [SMILES: *c1cccc2c1n(C)c(=O)n2C1CCC(=O)NC1=O]

In some embodiments, LBM is

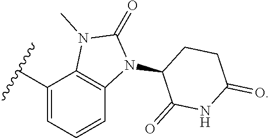 [SMILES: *c1cccc2c1n(C)c(=O)n2[C@H]1CCC(=O)NC1=O]

In some embodiments, LBM is

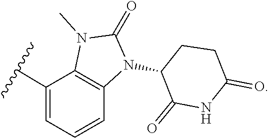 [SMILES: *c1cccc2c1n(C)c(=O)n2[C@@H]1CCC(=O)NC1=O]

In some embodiments, LBM is

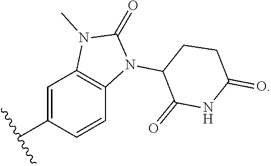 [SMILES: *c1ccc2c(c1)n(C)c(=O)n2C1CCC(=O)NC1=O]

In some embodiments, LBM is

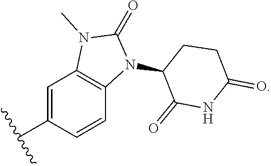 [SMILES: *c1ccc2c(c1)n(C)c(=O)n2[C@H]1CCC(=O)NC1=O]

In some embodiments, LBM is

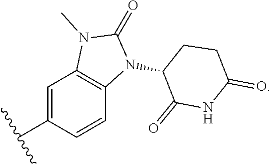 [SMILES: *c1ccc2c(c1)n(C)c(=O)n2[C@@H]1CCC(=O)NC1=O]

In some embodiments, LBM is

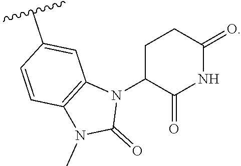 [SMILES: *c1ccc2c(c1)n(C1CCC(=O)NC1=O)c(=O)n2C]

In some embodiments, LBM is

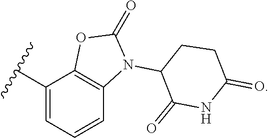 [SMILES: *c1cccc2c1oc(=O)n2C1CCC(=O)NC1=O]

In some embodiments, LBM is

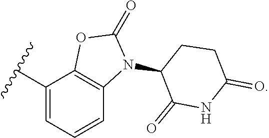 [SMILES: *c1cccc2c1oc(=O)n2[C@H]1CCC(=O)NC1=O]

In some embodiments, LBM is

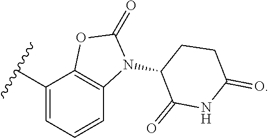 [SMILES: *c1cccc2c1oc(=O)n2[C@@H]1CCC(=O)NC1=O]

In some embodiments, LBM is

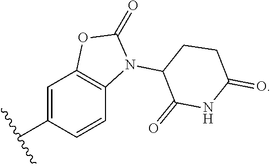 [SMILES: *c1ccc2c(c1)oc(=O)n2C1CCC(=O)NC1=O]

In some embodiments, LBM is

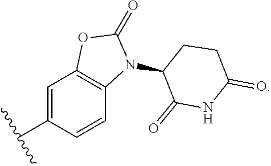 [SMILES: *c1ccc2c(c1)oc(=O)n2[C@H]1CCC(=O)NC1=O]

In some embodiments, LBM is

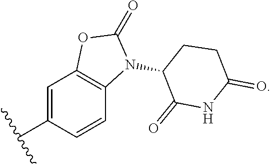 [SMILES: *c1ccc2c(c1)oc(=O)n2[C@@H]1CCC(=O)NC1=O]

In some embodiments, LBM is

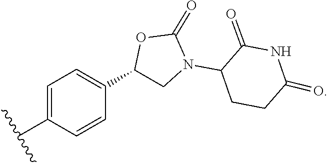 [SMILES: *c1ccc([C@H]2CN(C3CCC(=O)NC3=O)C(=O)O2)cc1]

In some embodiments, LBM is

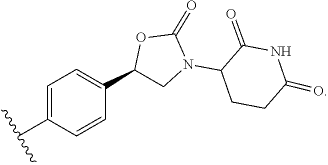 [SMILES: *c1ccc([C@@H]2CN(C3CCC(=O)NC3=O)C(=O)O2)cc1]

In some embodiments, LBM is

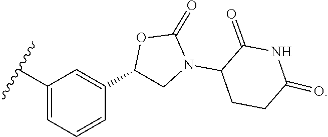 [SMILES: *c1cccc([C@H]2CN(C3CCC(=O)NC3=O)C(=O)O2)c1]

In some embodiments, LBM is

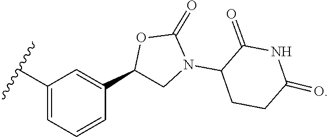 [SMILES: *c1cccc([C@@H]2CN(C3CCC(=O)NC3=O)C(=O)O2)c1]

In some embodiments, LBM is

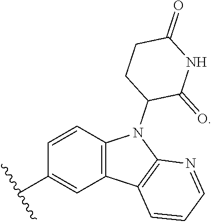 [SMILES: *c1ccc2c(c1)c1cccnc1n2C1CCC(=O)NC1=O]

In some embodiments, LBM is

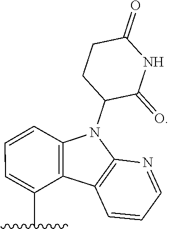 [SMILES: *c1cccc2c1c1cccnc1n2C1CCC(=O)NC1=O]

In some embodiments, LBM is

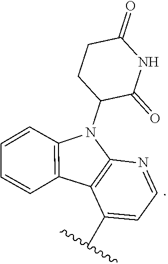 [SMILES: *c1ccnc2c1c1ccccc1n2C1CCC(=O)NC1=O]

In some embodiments, LBM is

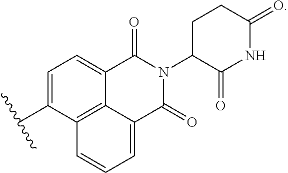 [SMILES: *c1ccc2c3c(cccc13)C(=O)N(C1CCC(=O)NC1=O)C2=O]

In some embodiments, LBM is

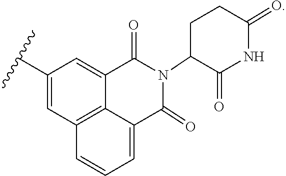 [SMILES: *c1cc2c3c(cccc3c1)C(=O)N(C1CCC(=O)NC1=O)C2=O]

In some embodiments, LBM is

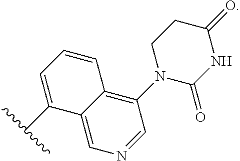 [SMILES: *c1cccc2c(N3CCC(=O)NC3=O)cncc12]

In some embodiments, LBM is

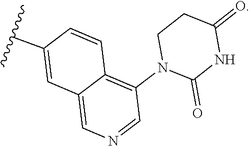 [SMILES: *c1ccc2c(N3CCC(=O)NC3=O)cncc2c1]

In some embodiments, LBM is

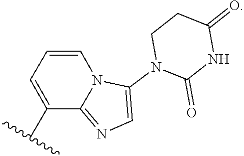 [SMILES: *c1cccn2c(N3CCC(=O)NC3=O)cnc12]

In some embodiments, LBM is

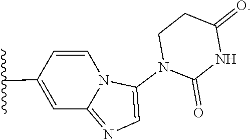 [SMILES: *c1ccn2c(N3CCC(=O)NC3=O)cnc2c1]

In some embodiments, LBM is

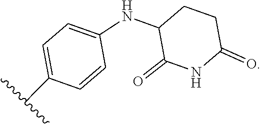 [SMILES: *c1ccc(NC2CCC(=O)NC2=O)cc1]

In some embodiments, LBM is

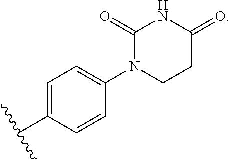 [SMILES: *c1ccc(N2CCC(=O)NC2=O)cc1]

In some embodiments, LBM is

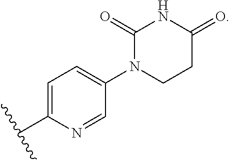 [SMILES: *c1ccc(N2CCC(=O)NC2=O)cn1]

In some embodiments, LBM is

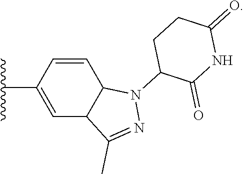 [SMILES: *C1=CC2C(C)=NN(C3CCC(=O)NC3=O)C2C=C1]

In some embodiments, LBM is

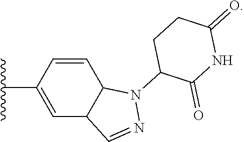 [SMILES: *C1=CC2C=NN(C3CCC(=O)NC3=O)C2C=C1]

In some embodiments, LBM is

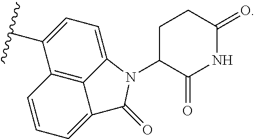 [SMILES: *c1ccc2c3c(cccc13)C(=O)N2C1CCC(=O)NC1=O]

In certain embodiments, the present invention provides a compound of Formula I, wherein LBM is a MDM2 (i.e. human double minute 2 or HIDM2) E3 ligase binding moiety thereby forming a compound of formula I-i-1, I-i-2, I-i-3, I-i-4, I-i-5, I-i-6, I-i-7, I-i-8, I-i-9, I-i-10, I-i-11, I-i-12, I-i-13, I-i-14, I-i-15, I-i-16, I-i-17, or I-i-18 respectively:

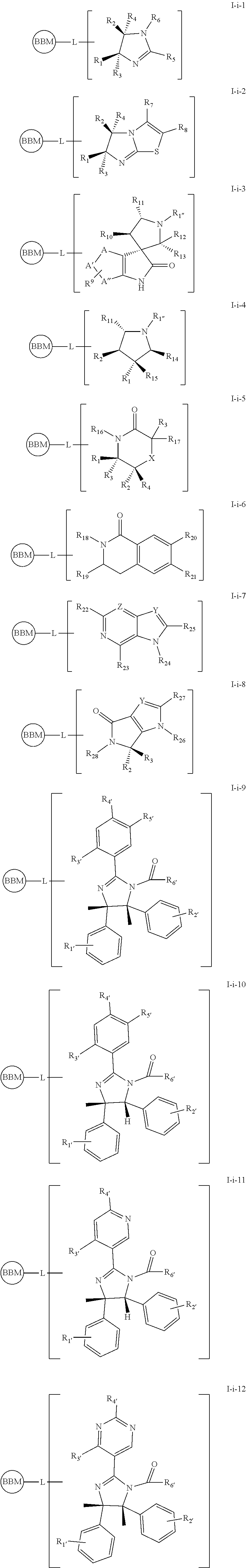

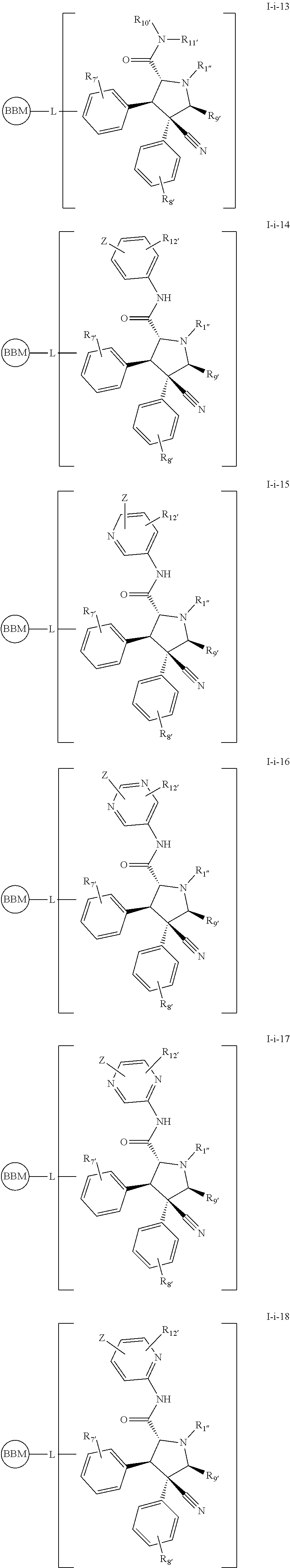

or a pharmaceutically acceptable salt thereof, wherein L and BBM are as defined above and described in embodiments herein, and wherein:
X is selected from —CR₂—, —O—, —S—, —S(O)—, —S(O)₂—, and —NR—;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-7 membered saturated, partially unsaturated, or heteroaryl ring having 0-3 heteroatoms, in addition to the atom from which they are attached, independently selected from nitrogen, oxygen, and sulfur.
Y and Z are independently selected from —CR═ and —N═;
Ring W is fused ring selected from benzo and a 5-6 membered heteroaryl with 1-4 heteroatoms independently selected from nitrogen, oxygen and sulfur;
R¹ and R² are independently an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R³ and R⁴ are independently selected from hydrogen and C₁₋₆ alkyl;
R⁵ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R⁶ is selected from hydrogen, —C(O)R, —C(O)OR, and —C(O)NR₂;
R⁷ is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R⁸ is selected from —C(O)R and R^{A};
R⁹ is a mono-, bis-, or tri-substituent on Ring W, wherein each of the substituents are independently selected from halogen and an optionally substituted C₁₋₆ aliphatic;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R¹¹ is —C(O)OR or —C(O)NR₂;
R¹² and R¹³ are independently selected from hydrogen and R^{A}, or;
R¹² and R¹³ are optionally taken together with their intervening atoms to form an optionally substituted 3-8 membered saturated, partially unsaturated, carbocyclic or heterocyclic ring having 1-3 heteroatoms independently selected from nitrogen, oxygen, and sulfur;
R¹⁴ is R^{A};
R¹⁵ is —CN;
R¹⁶ is selected from R^{A}, —OR, —(CR₂)₀₋₆—C(O)R, —(CR₂)₀₋₆—C(O)OR, —(CR₂)₀₋₆—C(O)NR₂, —(CR₂)₀₋₆—S(O)₂R, —(CR₂)₀₋₆—N(R)S(O)₂R, —(CR₂)₀₋₆—S(O)₂NR₂;
R¹⁷ is selected from —(CR₂)₀₋₆—C(O)NR₂;
R¹⁸ and R¹⁹ are independently selected from hydrogen and R^{A};
R²⁰ and R²¹ are independently selected from hydrogen, R^{A}, halogen, and —OR, or:
   R²⁰ and R²¹ are optionally taken together with their intervening atoms to form a fused 5-7 membered partially unsaturated carbocyclic or heterocyclic ring having 1-3 heteroatoms independently selected from nitrogen, oxygen, and sulfur, or a fused 5-6 membered heteroaryl ring having 1-3 heteroatoms independently selected from nitrogen, oxygen, and sulfur;
R²², R²³, R²⁵, and R²⁷ are independently selected from hydrogen, R^{A}, halogen, —C(O)R, —C(O)OR, —C(O)NR², —NR₂, —OR, —S(O)R, —S(O)₂R, —S(O)₂NR₂;
R²⁴, R²⁶, and R²⁸ are independently selected from hydrogen, R^{A}, —C(O)R, —C(O)OR, —C(O)NR₂, —S(O)R, —S(O)₂R, and —S(O)₂NR₂;
R^{1′} and R^{2′} are independently selected from halogen, —C≡CR, —CN, —CF₃, and —NO₂;
R^{3′} is —OR;
R^{4′}, R^{5′}, R^{6′} are independently selected from hydrogen, halogen, R^{A}, —CN, —CF₃, —NR₂, —OR, —SR, and —S(O)₂R;
R^{7′} is a mono-, bis-, or tri-substituent, wherein each of the substituents are independently selected from halogen;
R⁸ is a mono-, bis-, or tri-substituent, wherein each of the substituents are independently selected from hydrogen, halogen, R^{A}, —CN, —C≡CR, —NO₂, and —OR;
R^{9′} is R^{A};
Z¹ is selected from hydrogen, halogen, and —OR;
R^{10′} and R^{11′} are independently selected from hydrogen and R^{A};
R^{12′} is selected from —C(O)R, —C(O)OR, —C(O)NR₂, —OR, —S(O)₂R, —S(O)₂NR₂, and —S(O)R; and
R^{1″} is selected from hydrogen and R^{A}.

In certain embodiments, the present invention provides a compound of Formula I, wherein LBM is a MDM2 (i.e. human double minute 2 or HDM2) E3 ligase binding moiety thereby forming a compound of formula I-i-19, I-i-20, or I-i-21 respectively:

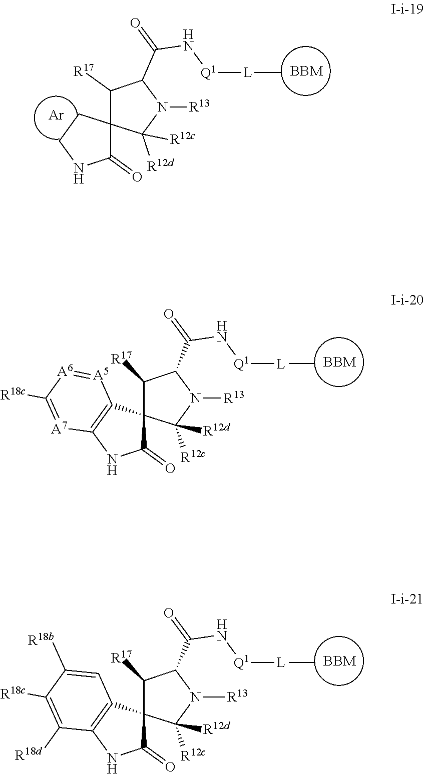

or a pharmaceutically acceptable salt thereof, wherein L and BBM are as defined above and described in embodiments herein, and wherein:
R^{1″} is selected from hydrogen and R^{A};
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R¹⁰ is selected from an optionally substituted monocyclic or bicyclic ring selected from phenyl, a 5-10 membered aryl, and a 5-10 membered heteroaryl containing 1-4 heteroatoms independently selected from nitrogen, oxygen, and sulfur;
R₁₂ and R₁₃ are each independently selected from hydrogen and R^{A}, or:
   R¹² and R¹³ are optionally taken together with their intervening atoms to form an optionally substituted 4-8 membered saturated, partially unsaturated, carbocyclic or heterocyclic ring having 1-3 heteroatoms independently selected from nitrogen, oxygen, and sulfur;
A⁵ is selected from —C(R^{18a})═ and —N═;
A⁶ is selected from —C(R^{18b})═ and —N═;
A⁷ is selected from —C(R^{18d})═ and —N═;
R^{18a}, R^{18b}, R^{18c}, and R^{18d} are each independently selected from hydrogen, halogen, R^{A}, and —OR;
each R is independently hydrogen or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring W is an optionally substituted fused ring selected from benzo and a 5-6 membered heteroaryl with 1-4 heteroatoms independently selected from nitrogen, oxygen and sulfur; and
Q¹ is and optionally substituted bivalent group selected from alkylenyl, phenylenyl, heteroarylenyl, cycloalkylenyl, and heterocyclenyl.

In certain embodiments, the present invention provides a compound of formula I, wherein LBM is an IAP E3 ubiquitin ligase binding moiety thereby forming a compound of formula I-j-1, I-j-2, I-j-3, or I-j-4 respectively:

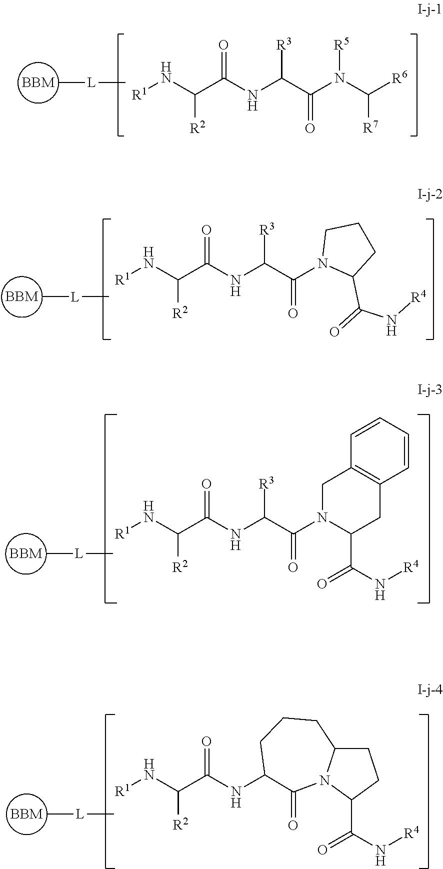

or a pharmaceutically acceptable salt thereof, wherein L and BBM are as defined above and described in embodiments herein, and wherein each of the variables R¹, R², R³, R⁴, R⁵, R⁶, and R⁷, is as defined and described in WO 2017/011590 and US 2017/0037004, the entirety of each of which is herein incorporated by reference.

In certain embodiments, the present invention provides a compound of formula I, wherein LBM is an LAP binding moiety thereby forming a compound of formula I-k-1:

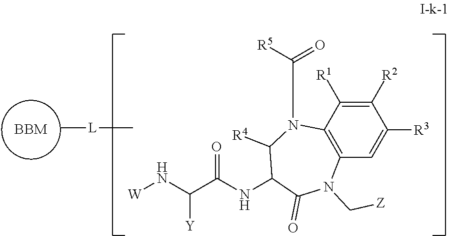

or a pharmaceutically acceptable salt thereof, wherein L and BBM are as defined above and described in embodiments herein, and wherein each of the variables W, Y, Z, R¹, R², R³, R⁴, and R⁵ is as described and defined in WO 2014/044622, US 2015/0225449, WO 2015/071393, and US 2016/0272596, the entirety of each of which is herein incorporated by reference.

In certain embodiments, the present invention provides a compound of formula I, wherein LBM is a DCAF16 binding moiety thereby forming a compound of formula I-k-2:

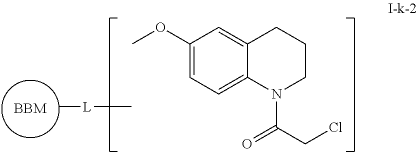

or a pharmaceutically acceptable salt thereof as described and defined in Zhang, X. et al., bioRxiv (doi: https://doi.org/10.1101/443804), the entirety of each of which is herein incorporated by reference, and wherein L and BBM are as defined above and described in embodiments herein.

In certain embodiments, the present invention provides a compound of formula I, wherein LBM is a RNF114 binding moiety thereby forming a compound of formula I-k-3:

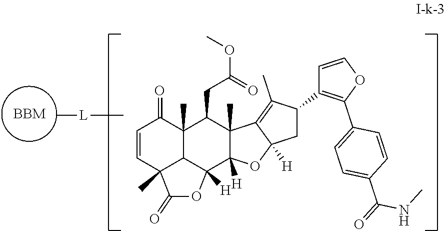

or a pharmaceutically acceptable salt thereof, as described and defined in Spradin, J. N. et al., bioRxiv (doi: https://doi.org/10.1101/436998), the entirety of each of which is herein incorporated by reference, and wherein L and BBM are as defined above and described in embodiments herein.

In certain embodiments, the present invention provides a compound of formula I, wherein LBM is a RNF4 binding moiety thereby forming a compound of formula I-k-4:

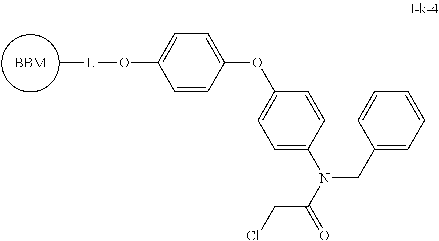

or a pharmaceutically acceptable salt thereof, as described and defined in Ward, C. C., et al., bioRxiv (doi: https://doi.org/10.1101/439125), the entirety of each of which is herein incorporated by reference, and wherein L and BBM are as defined above and described in embodiments herein.

In certain embodiments, the present invention provides a compound of formula I, wherein LBM is a E3 ubiquitin ligase (cereblon) binding moiety thereby forming a compound of formula I-l-1, I-l-2, I-l-3, or I-l-4:

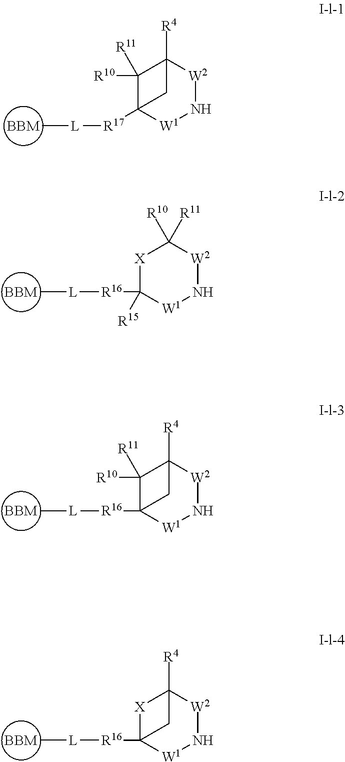

or a pharmaceutically acceptable salt thereof, wherein L and BBM are as defined above and described herein, and wherein each of the variables R⁴, R¹⁰, R¹¹, R¹⁵, R¹⁶, R¹⁷, W¹, W², and X is as defined in WO 2019/099868 which is herein incorporated by reference in its entirety, and wherein

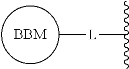

is attached to R¹⁷ or R¹⁶ at the site of attachment of R¹² as defined in WO 2018/237026, such that

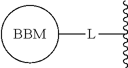

takes the place of the R¹² substituent.

In certain embodiments, the present invention provides a compound of formula I, wherein LBM is a cereblon E3 ubiquitin ligase binding moiety, a DCAF15 E3 ubiquitin ligase binding moiety, or a VHL E3 ubiquitin ligase binding moiety; thereby forming a compound of formula I-m-1, I-m-2, or I-m-3:

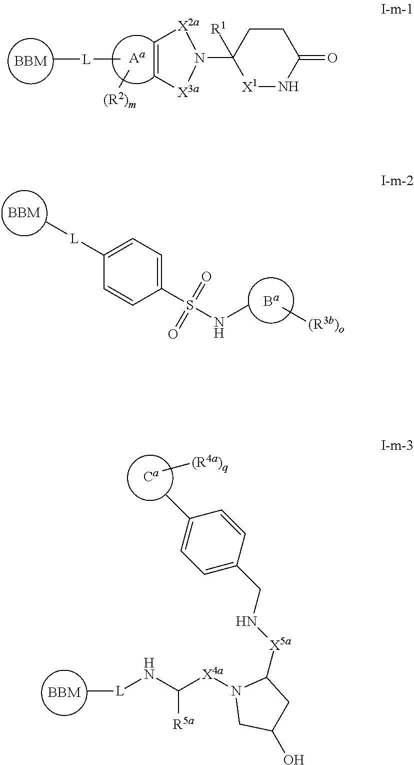

or a pharmaceutically acceptable salt thereof, wherein L and BBM is as defined above and described in embodiments herein, and wherein:
each of X¹, X^{2a}, and X^{3a} is independently a bivalent moiety selected from a covalent bond, —CH₂—, —C(O)—, —C(S)—, or

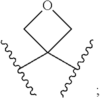 [SMILES: *C1(*)COC1]

each of X^{4a} and X^{5a} is independently a bivalent moiety selected from —CH₂—, —C(O)—, —C(S)—, or

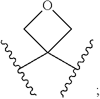 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R^{5a} is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen and sulfur, or 5-membered heteroaryl with 1-3 heteroatoms independently selected from nitrogen, oxygen and sulfur;
Ring B^{a} is selected from 6-membered aryl containing 0-2 nitrogen atoms or a 8-10 membered bicyclic heteroaryl having 1-5 heteroatoms independently selected from nitrogen, oxygen, and sulfur;
Ring C^{a} is a selected from 6-membered aryl containing 0-2 nitrogen atoms or a 5-membered heteroaryl with 1-3 heteroatoms independently selected from nitrogen, oxygen and sulfur;
m is 0, 1, 2, 3 or 4;
is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of formula I-m-1, wherein LBM is an E3 ubiquitin ligase (cereblon) binding moiety thereby forming a compound of formula I-m-4 or I-m-5:

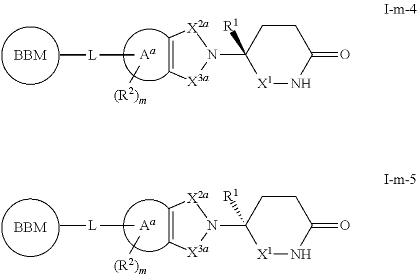

or a pharmaceutically acceptable salt thereof, wherein BBM, L, Ring A^{a}, X¹, X^{2a}, X^{3a}, R¹, R² and m are as described above.

As defined above and described herein, each of X¹, X^{2a}, and X^{3a} is independently a bivalent moiety selected from a covalent bond, —CH₂—, —C(O)—, —C(S)—, or

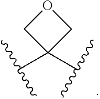 [SMILES: *C1(*)COC1]

In some embodiments, X¹ is a covalent bond, —CH₂—, —C(O)—, —C(S)—, or

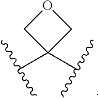 [SMILES: *C1(*)COC1]

In some embodiments, X¹ is selected from those depicted in Table 1, below.

In some embodiments, X^{2a} is a covalent bond, —CH₂—, —C(O)—, —C(S)—, or

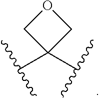 [SMILES: *C1(*)COC1]

In some embodiments, X^{2a} is selected from those depicted in Table 1, below.

In some embodiments, X^{3a} is a covalent bond, —CH₂—, —C(O)—, —C(S)—, or

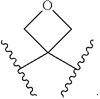 [SMILES: *C1(*)COC1]

In some embodiments, X^{3a} is selected from those depicted in Table 1, below.

As defined above and described herein, each of X^{4a} and X^{5a} is independently a bivalent moiety selected from —CH₂—, —C(O)—, —C(S)—, or

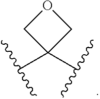 [SMILES: *C1(*)COC1]

In some embodiments, X^{4a} is —CH₂—, —C(O)—, —C(S)—, or

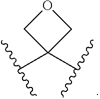 [SMILES: *C1(*)COC1]

In some embodiments, X^{4a} is selected from those depicted in Table 1, below.

In some embodiments, X^{5a} is —CH₂—, —C(O)—, —C(S)—, or

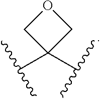 [SMILES: *C1(*)COC1]

In some embodiments, X^{5a} is selected from those depicted in Table 1, below.

As defined above and described herein, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is hydrogen. In some embodiments, R¹ is deuterium. In some embodiments, R¹ is halogen. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is —NR₂. In some embodiments, R¹ is optionally substituted C₁₋₄ aliphatic.

In some embodiments, R¹ is selected from those depicted in Table 1, below.

As defined above and described herein, each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is selected from those depicted in Table 1, below.

In some embodiments, R^{3b} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{3b} is methyl.

In some embodiments, R^{3b} is selected from those depicted in Table 1, below.

In some embodiments, R^{4a} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{4a} is methyl.

In some embodiments, R^{4a} is selected from those depicted in Table 1, below.

As defined above and described herein, R^{5a} is hydrogen or C₁₋₆ aliphatic.

In some embodiments, R^{5a} is t-butyl.

In some embodiments, R^{5a} is selected from those depicted in Table 1, below.

As defined above and described herein, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁶ is an optionally substituted C₁₋₆ aliphatic group. In some embodiments, R⁶ is an optionally substituted phenyl. In some embodiments, R⁶ is an optionally substituted 4-7 membered saturated or partially unsaturated heterocyclic ring having 1-2 heteroatoms independently selected from nitrogen, oxygen, and sulfur. In some embodiments, R⁶ is an optionally substituted 5-6 membered heteroaryl ring having 1-4 heteroatoms independently selected from nitrogen, oxygen, and sulfur.

In some embodiments, R⁶ is selected from those depicted in Table 1, below.

As defined above and described herein, Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen and sulfur, or 5-membered heteroaryl with 1-3 heteroatoms independently selected from nitrogen, oxygen and sulfur.

In some embodiments Ring A^{a} is a fused 6-membered aryl containing 0-2 nitrogen atoms. In some embodiments Ring A^{a} is a fused 5 to 7-membered partially saturated carbocyclyl. In some embodiments Ring A^{a} is a fused 5 to 7-membered partially saturated heterocyclyl with 1-2 heteroatoms independently selected from nitrogen, oxygen and sulfur. In some embodiments Ring A^{a} is a fused 5-membered heteroaryl with 1-3 heteroatoms independently selected from nitrogen, oxygen and sulfur.

In some embodiments, Ring A^{a} is a fused phenyl.

In some embodiments, Ring A^{a} is selected from those depicted in Table 1, below.

As defined above and described herein, Ring B^{a} is selected from 6-membered aryl containing 0-2 nitrogen atoms or a 8-10 membered bicyclic heteroaryl having 1-5 heteroatoms independently selected from nitrogen, oxygen, and sulfur.

In some embodiments, Ring B^{a} is a 6-membered aryl containing 0-2 nitrogen atoms. In some embodiments, Ring B^{a} is a 8-10 membered bicyclic heteroaryl having 1-5 heteroatoms independently selected from nitrogen, oxygen, and sulfur.

In some embodiments, Ring B^{a} is

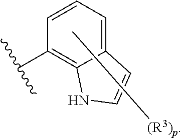

In some embodiments, Ring B^{a} is selected from those depicted in Table 1, below.

As defined above and described herein, Ring C^{a} is selected from 6-membered aryl containing 0-2 nitrogen atoms or a 5-membered heteroaryl with 1-3 heteroatoms independently selected from nitrogen, oxygen and sulfur.

In some embodiments, Ring C^{a} is a 6-membered aryl containing 0-2 nitrogen atoms. In some embodiments, Ring C^{a} is a 5-membered heteroaryl with 1-3 heteroatoms independently selected from nitrogen, oxygen and sulfur.

In some embodiments, Ring C^{a} is

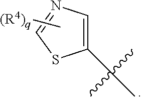

In some embodiments, Ring C^{a} is selected from those depicted in Table 1, below.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In some embodiments, m is selected from those depicted in Table 1, below.

In some embodiments, o is selected from those depicted in Table 1, below.

As defined above and described herein, o is 0, 1, 2, 3 or 4.

In some embodiments, o is 0. In some embodiments, o is 1. In some embodiments, o is 2. In some embodiments, o is 3. In some embodiments, o is 4.

In some embodiments, o is selected from those depicted in Table 1, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is phenyl. In some embodiments, R is a 4-7 membered saturated or partially unsaturated heterocyclic having 1-2 heteroatoms independently selected from nitrogen, oxygen, and sulfur. In some embodiments, R is a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is selected from those depicted in Table 1, below.

In certain embodiments, the present invention provides a compound of formula I, wherein LBM is a VHL E3 ubiquitin ligase binding moiety, thereby forming a compound of formula I-n:

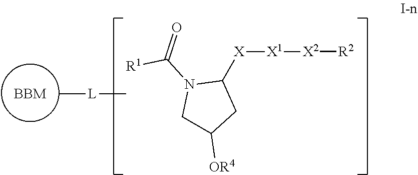

or a pharmaceutically acceptable salt thereof, wherein L and BBM is as defined above and described in embodiments herein, and wherein:
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
R^{A} is an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
R² is hydrogen, halogen, —CN,

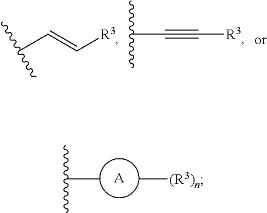

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, halogen, C₁₋₆alkyl, C₁₋₆haloalkyl, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, —N(R)SO₂R, or R^{A}; or
   two R³ groups are optionally taken together to form an optionally substituted 5-7 membered partially unsaturated or aryl fused ring having 0-2 heteroatoms independently selected from nitrogen, oxygen, and sulfur;
R⁴ is hydrogen, —C(O)R, —C(O)OR, —C(O)NR₂, —P(O)R₂, —P(O)(OR)₂, —(CR₂)₁₋₃OP(O)R₂, —(CR₂)₁₋₃OP(O)(OR)₂, or R^{A};
n is 0, 1, 2, 4, or 5.

As defined above and described herein, in some embodiments, X is —C(O)—, —C(O)NR—, —SO₂—, —SO₂NR—, or an optionally substituted 5-membered heterocyclic ring.

In some embodiments, X is —C(O)—. In some embodiments, X is —C(O)NR—. In some embodiments, X is —SO₂—. In some embodiments, X is —SO₂NR—. In some embodiments, X is an optionally substituted 5-membered heterocyclic ring.

In some embodiments, X is —C(O)NH—. In some embodiments, X is

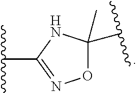 [SMILES: *C1=NOC(*)(C)N1]

In some embodiments, X is selected from those depicted in Table 1, below.

As defined above and described herein, in some embodiments, X¹ is a bivalent group selected from a covalent bond, —O—, —C(O)—, —C(S)—, —C(R)₂—, —NR—, —S(O)—, or —SO₂—.

In some embodiments, X¹ is a covalent bond. In some embodiments, X¹ is —O—. In some embodiments, X¹ is —C(O)—. In some embodiments, X¹ is —C(S)—. In some embodiments, X¹ is —C(R)₂—. In some embodiments, X¹ is —NR—. In some embodiments, X¹ is —S(O)—. In some embodiments, X¹ is —SO₂—.

In some embodiments, X¹ is

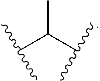 [SMILES: *C(*)C]

In some embodiments, X¹ is

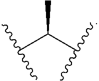

In some embodiments, X¹ is

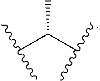

In some embodiments, X¹ is

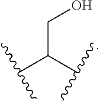 [SMILES: *C(*)CO]

In some embodiments, X¹ is

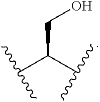

In some embodiments, X¹ is

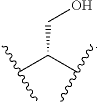

In some embodiments, X¹ is selected from those depicted in Table 1, below.

As defined above and described herein, in some embodiments, X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur.

In some embodiments, X² is an optionally substituted C₁₋₆ saturated or unsaturated alkylene. In some embodiments, X² is an optionally substituted phenylenyl. In some embodiments, X² is an optionally substituted 5-6 membered heteroarylenyl containing 1-4 heteroatoms independently selected from nitrogen, oxygen, and sulfur. In some embodiments, X² is an optionally substituted 4-11 membered saturated or partially unsaturated monocyclic, bicyclic, bridged bicyclic, or spirocyclic carbocyclylenyl. In some embodiments, X² is an optionally substituted 4-11 membered saturated or partially unsaturated monocyclic, bicyclic, bridged bicyclic, or spirocyclic heterocyclylenyl with 1-3 heteroatoms independently selected from nitrogen, oxygen, and sulfur.

In some embodiments, X² is

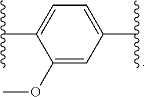 [SMILES: *c1ccc(*)c(OC)c1]

In some embodiments, X² is

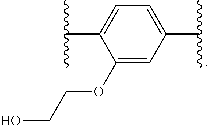 [SMILES: *c1ccc(*)c(OCCO)c1]

In some embodiments, X² is selected from those depicted in Table 1, below.

As defined above and described herein, in some embodiments, R¹ is R^{A}, —C(R)₂R^{A}, —OR, —SR, —N(R)₂, —C(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R.

In some embodiments, R¹ is R^{A}. In some embodiments, R¹ is —C(R)₂R^{A}. In some embodiments, R¹ is —OR. In some embodiments, R¹ is —SR. In some embodiments, R¹ is —N(R)₂. In some embodiments, R¹ is —C(R)₂OR. In some embodiments, R¹ is —C(R)₂N(R)₂. In some embodiments, R¹ is —C(R)₂NRC(O)R. In some embodiments, R¹ is —C(R)₂NRC(O)N(R)₂. In some embodiments, R¹ is —NRC(O)OR. In some embodiments, R¹ is —NRC(O)R. In some embodiments, R¹ is —NRC(O)N(R)₂. In some embodiments, R¹ is —NRSO₂R.

In some embodiments, R¹ is

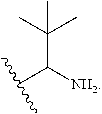 [SMILES: *C(N)C(C)(C)C]

In some embodiments, R¹ is

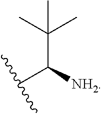 [SMILES: *[C@H](N)C(C)(C)C]

In some embodiments, R¹ is

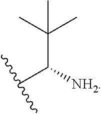 [SMILES: *[C@@H](N)C(C)(C)C]

In some embodiments, R¹ is

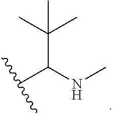 [SMILES: *C(NC)C(C)(C)C]

In some embodiments, R¹ is

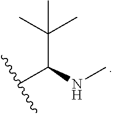 [SMILES: *[C@H](NC)C(C)(C)C]

In some embodiments, R¹ is

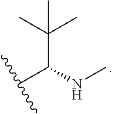 [SMILES: *[C@@H](NC)C(C)(C)C]

In some embodiments, R¹ is

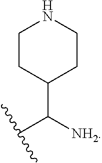 [SMILES: *C(N)C1CCNCC1]

In some embodiments, R¹ is

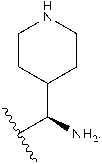 [SMILES: *[C@H](N)C1CCNCC1]

In some embodiments, R¹ is

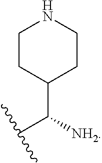 [SMILES: *[C@@H](N)C1CCNCC1]

In some embodiments, R¹ is

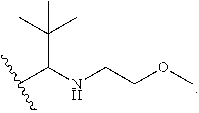 [SMILES: *C(NCCOC)C(C)(C)C]

In some embodiments, R¹ is

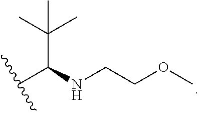 [SMILES: *[C@H](NCCOC)C(C)(C)C]

In some embodiments, R¹ is

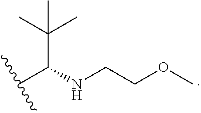 [SMILES: *[C@@H](NCCOC)C(C)(C)C]

In some embodiments, R¹ is

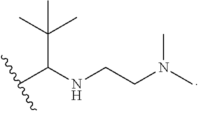 [SMILES: *C(NCCN(C)C)C(C)(C)C]

In some embodiments, R¹ is

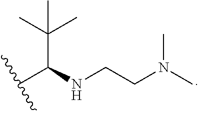 [SMILES: *[C@H](NCCN(C)C)C(C)(C)C]

In some embodiments, R¹ is

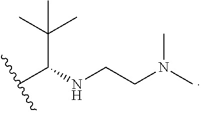 [SMILES: *[C@@H](NCCN(C)C)C(C)(C)C]

In some embodiments, R¹ is

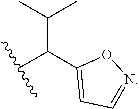 [SMILES: *C(c1ccno1)C(C)C]

In some embodiments, R¹ is

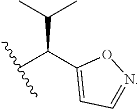 [SMILES: *[C@H](c1ccno1)C(C)C]

In some embodiments, R¹ is

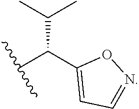 [SMILES: *[C@@H](c1ccno1)C(C)C]

In some embodiments, R¹ is

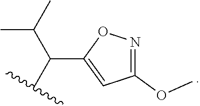 [SMILES: *C(c1cc(OC)no1)C(C)C]

In some embodiments, R¹ is

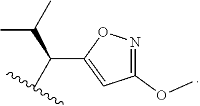 [SMILES: *[C@H](c1cc(OC)no1)C(C)C]

In some embodiments, R¹ is

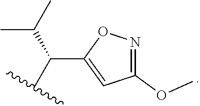 [SMILES: *[C@@H](c1cc(OC)no1)C(C)C]

In some embodiments, R¹ is

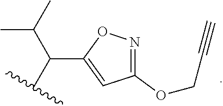 [SMILES: *C(c1cc(OCC#C)no1)C(C)C]

In some embodiments, R¹ is

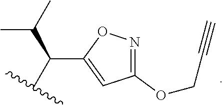 [SMILES: *[C@H](c1cc(OCC#C)no1)C(C)C]

In some embodiments, R¹ is

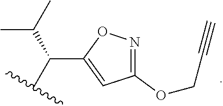 [SMILES: *[C@@H](c1cc(OCC#C)no1)C(C)C]

In some embodiments, R¹ is

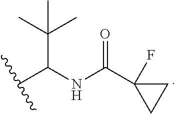 [SMILES: *C(NC(=O)C1(F)CC1)C(C)(C)C]

In some embodiments, R¹ is

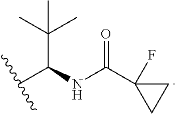 [SMILES: *[C@H](NC(=O)C1(F)CC1)C(C)(C)C]

In some embodiments, R¹ is

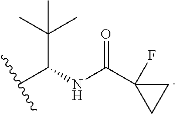 [SMILES: *[C@@H](NC(=O)C1(F)CC1)C(C)(C)C]

In some embodiments, R¹ is

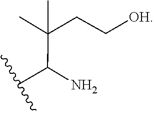 [SMILES: *C(N)C(C)(C)CCO]

In some embodiments, R¹ is

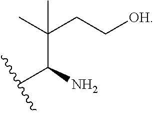 [SMILES: *[C@H](N)C(C)(C)CCO]

In some embodiments, R¹ is

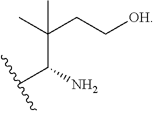 [SMILES: *[C@@H](N)C(C)(C)CCO]

In some embodiments, R¹ is

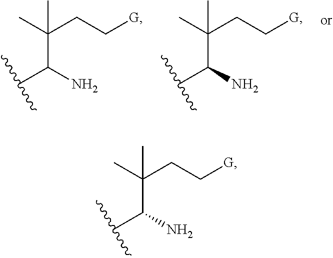

wherein G is —OH, —O(CH₂)₁₋₅CO₂R (e.g., —OCH₂CO₂H, etc.)), —OP(O)(OR)₂ (e.g., —OP(O)(OH)₂, etc.)), —O(CH₂)₁₋₅P(O)(OR)₂ (e.g., —O(CH₂)₂P(O)(OH)₂, etc.)),

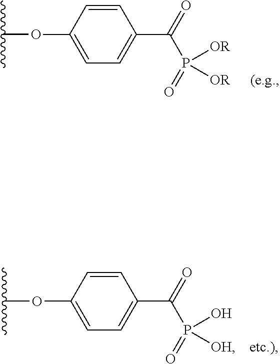

### or —NR₂

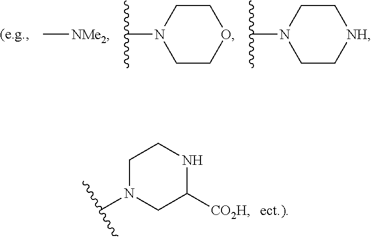

In some embodiments, R¹ is selected from those depicted in Table 1, below.

As defined above and described herein, in some embodiments, R² is hydrogen, halogen, —CN,

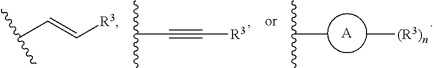

In some embodiments, R² is hydrogen. In some embodiments, R² is halogen. In some embodiments, R² is —CN. In some embodiments, R² is

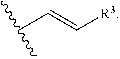

In some embodiments, R² is

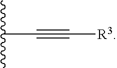

In some embodiments, R² is

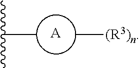

In some embodiments, R² is floro. In some embodiments, R² is chloro. In some embodiments, R² is

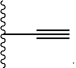 [SMILES: *C#C]

In some embodiments, R² is selected from those depicted in Table 1, below.

As defined above and described herein, in some embodiments, Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur.

In some embodiments, Ring A is phenyl. In some embodiments, Ring A is a 5-6 membered heteroaryl containing 1-4 heteroatoms independently selected from nitrogen, oxygen, and sulfur. In some embodiments, Ring A is 4 to 9-membered saturated or partially unsaturated monocyclic, bicyclic, bridged bicyclic, or spirocyclic carbocyclyl. In some embodiments, Ring A is a 4 to 9-membered saturated or partially unsaturated monocyclic, bicyclic, bridged bicyclic, or spirocyclic heterocyclyl with 1-3 heteroatoms independently selected from nitrogen, oxygen, and sulfur.

In some embodiments, Ring A is

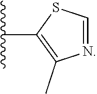 [SMILES: *c1scnc1C]

In some embodiments, Ring A is

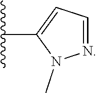 [SMILES: *c1ccnn1C]

In some embodiments, Ring A is selected from those depicted in Table 1, below.

As defined above and described herein, in some embodiments, each of R³ is independently hydrogen, halogen, C₁₋₆alkyl, C₁₋₆haloalkyl, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R), —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, —N(R)SO₂R, or R^{A}, or two R³ groups are optionally taken together to form an optionally substituted 5-7 membered partially unsaturated or aryl fused ring having 0-2 heteroatoms independently selected from nitrogen, oxygen, and sulfur.

In some embodiments, R³ is hydrogen. In some embodiments, R³ is R^{A}. In some embodiments, R³ is halogen. In some embodiments, R³ is C₁₋₆alkyl. In some embodiments, R³ is C₁₋₆haloalkyl (e.g., —CF₃, —CHF₂, etc.). In some embodiments, R³ is —CN. In some embodiments, R³ is —NO₂. In some embodiments, R³ is —OR. In some embodiments, R³ is —SR. In some embodiments, R³ is —N(R)₂. In some embodiments, R³ is —Si(R)₃. In some embodiments, R³ is —SO₂R. In some embodiments, R³ is —SO₂NR₂. In some embodiments, R³ is —S(O)R. In some embodiments, R³ is —C(O)R. In some embodiments, R³ is —C(O)OR. In some embodiments, R³ is —C(O)N(R)₂. In some embodiments, R³ is —C(O)N(R)OR. In some embodiments, R³ is —C(R)₂NRC(O)R. In some embodiments, R³ is —C(R)₂NRC(O)N(R)₂. In some embodiments, R³ is —OC(O)R. In some embodiments, R³ is —OC(O)N(R)₂. In some embodiments, R³ is —OP(O)(R)₂. In some embodiments, R³ is —OP(O)(OR)₂. In some embodiments, R³ is —OP(O)(OR)N(R)₂. In some embodiments, R³ is —OP(O)(N(R)₂)₂—. In some embodiments, R³ is —N(R)C(O)OR. In some embodiments, R³ is —N(R)C(O)R. In some embodiments, R³ is —NRC(O)N(R)₂. In some embodiments, R³ is —N(R)SO₂R. In some embodiments, R³ is —NP(O)(R)₂. In some embodiments, R³ is —N(R)P(O)(OR)₂. In some embodiments, R³ is —N(R)P(O)(OR)N(R)₂. In some embodiments, R³ is —N(R)P(O)(N(R)₂)₂. In some embodiments, R³ is —N(R)SO₂R. In some embodiments, R³ is R^{A}. In some embodiments, two R³ groups are optionally taken together to form an optionally substituted 5-7 membered partially unsaturated or aryl fused ring having 0-2 heteroatoms independently selected from nitrogen, oxygen, and sulfur.

In some embodiments, R³ is methyl.

In some embodiments, R³ is selected from those depicted in Table 1, below.

As defined above and described herein, R⁴ is hydrogen, —C(O)R, —C(O)OR, —C(O)NR₂, —P(O)R₂, —P(O)(OR)₂, —(CR₂)₁₋₃OP(O)R₂, —(CR₂)₁₋₃OP(O)(OR)₂, or R^{A}.

In some embodiments, R⁴ is hydrogen. In some embodiments, R⁴ is —C(O)R. In some embodiments, R⁴ is —C(O)OR. In some embodiments, R⁴ is —C(O)NR₂. In some embodiments, R⁴ is —P(O)R₂. In some embodiments, R⁴ is —P(O)(OR)₂. In some embodiments, R⁴ is —(CR₂)₁₋₃OP(O)R₂. In some embodiments, R⁴ is —(CR₂)₁₋₃OP(O)(OR)₂. In some embodiments, R⁴ is R^{A}.

In some embodiments, R⁴ is selected from those depicted in Table 1, below.

As defined above and described herein, in some embodiments, n is 0, 1, 2, 4, or 5.

In some embodiments, n is 0. In some embodiments, n is 1. In some embodiments, n is 2. In some embodiments, n is 3. In some embodiments, n is 4. In some embodiments, n is 5.

In some embodiments, n is selected from those depicted in Table 1, below.

In certain embodiments, the present invention provides a compound of formula I-aa-1:

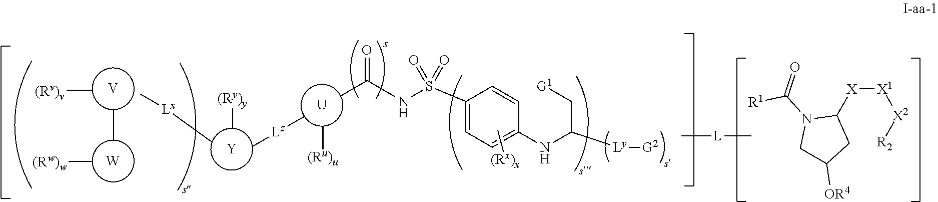

or a pharmaceutically acceptable salt, wherein:
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Ring W is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 5-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
G¹ is —S-aryl, —S-heteroaryl, or —R^{A};
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
G² is hydrogen, halogen, —CN, —OR, —SR, —N(R)₂, —S(O)₂R, —S(O)₂N(R)₂, —C(O)R, —C(O)OR, or

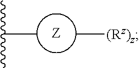

each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom arc optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
Ring Z is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
R^{u}, R^{v}, R^{w}, R^{x}, R^{y}, and R^{z} are, independently, hydrogen, halogen, C₁₋₆alkyl, C₁₋₆haloalkyl, —CN, —NO₂, —OR, —SR, —N(R)₂, —Si(R)₃, —S(O)₂R, —S(O)₂N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, —NRS(O)₂R, or R^{A};
L^{x}, L^{y}, and L^{z} are, independently, a covalent bond or a bivalent, saturated or partially unsaturated, straight or branched C₁₋₅ hydrocarbon chain, wherein 0-3 methylene units of L^{x}, L^{y}, and L^{z} are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
s, s″, and s′″ are, independently, 0 or 1;
s′ is 1 or 2;
u, v, w, x, y, and z are, independently, 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—

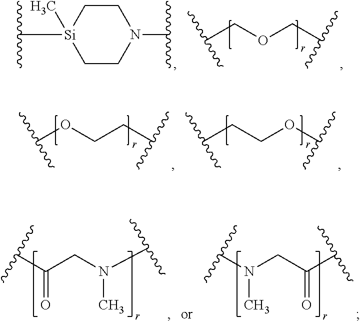

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R² is hydrogen, halogen, —CN,

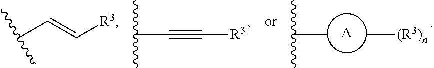

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, halogen, C₁₋₆alkyl, C₁₋₆haloalkyl, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, —N(R)SO₂R, or R^{A}; or
   two R³ groups are optionally taken together to form an optionally substituted 5-7 membered partially unsaturated or aryl fused ring having 0-2 heteroatoms independently selected from nitrogen, oxygen, and sulfur; and
R⁴ is hydrogen, —C(O)R, —C(O)OR, —C(O)NR₂, —P(O)R₂, —P(O)(OR)₂, —(CR₂)₁₋₃OP(O)R₂, —(CR₂)₁₋₃OP(O)(OR)₂, or R^{A};
n is 0, 1, 2, 4, or 5.

In certain embodiments, the present invention provides a compound of formula I-aa-1, wherein R¹ is

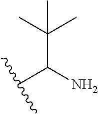 [SMILES: *C(N)C(C)(C)C]

(where one of the hydrogen atoms of the NH₂ group is replaced with -L-), s″ and s′″ are 1, and G¹ is —SPh as shown, to provide a compound of formula I-aa-2:

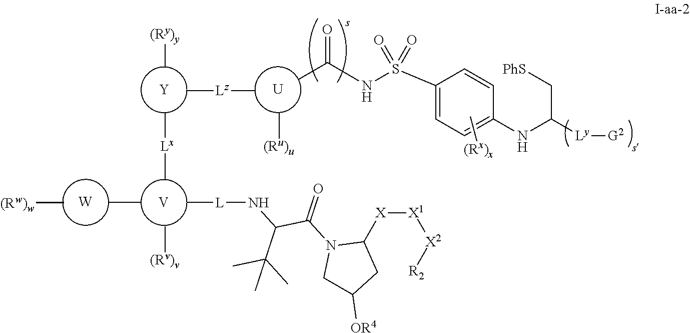

or a pharmaceutically acceptable salt thereof, wherein each of L, L^{x}, L^{y}, L^{z}, G², R², R^{u}, R^{v}, R^{y}, R^{w}, R^{x}, R^{y}, Ring U, Ring V, Ring W, Ring Y, X, X¹, X², s, s′, u, v, w, x, and y is as defined above and described in embodiments herein, both singly and in combination.

In certain embodiments, the present invention provides a compound of formula I-aa-1, wherein R¹ is

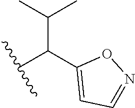 [SMILES: *C(c1ccno1)C(C)C]

(where one of the hydrogen atoms of the isoxazolyl group is replaced with -L-), s″ and s′″ are 1, and G¹ is —SPh as shown, to provide a compound of formula I-aa-3:

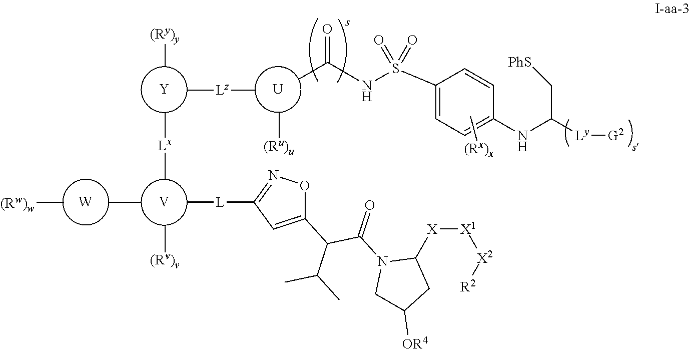

or a pharmaceutically acceptable salt thereof, wherein each of L, L^{x}, L^{y}, L^{z}, G², R², R⁴, R^{u}, R^{v}, R^{w}, R^{x}, R^{y}, Ring U, Ring V, Ring W, Ring Y, X, X¹, X², s, s′, u, v, w, x, and y is as defined above and described in embodiments herein, both singly and in combination.

In certain embodiments, the present invention provides a compound of formula I-aa-1, wherein R¹ is

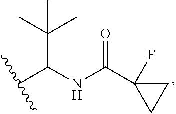 [SMILES: *C(NC(=O)C1(F)CC1)C(C)(C)C]

X² is phenylenyl, s″ and s″ are 1, and G¹ is —SPh as shown, to provide a compound of formula I-aa-4:

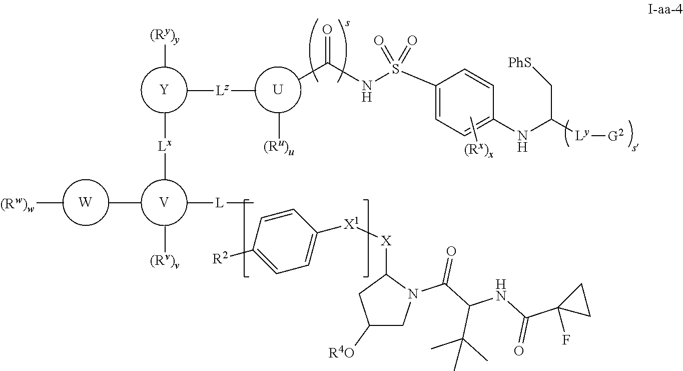

or a pharmaceutically acceptable salt thereof, wherein each of L, L^{x}, L^{y}, L^{z}, G², R², R⁴, R^{u}, R^{v}, R^{w}, R^{x}, R^{y}, Ring U, Ring V, Ring W, Ring Y, X, X¹, s, s′, u, v, w, x, and y is as defined above and described in embodiments herein, both singly and in combination.

In certain embodiments, the present invention provides a compound of formula I-aa-1, wherein R² is

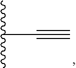 [SMILES: *C#C]

s″ and s′″ are 1, and G¹ is —SPh as shown, to provide a compound of formula I-aa-5:

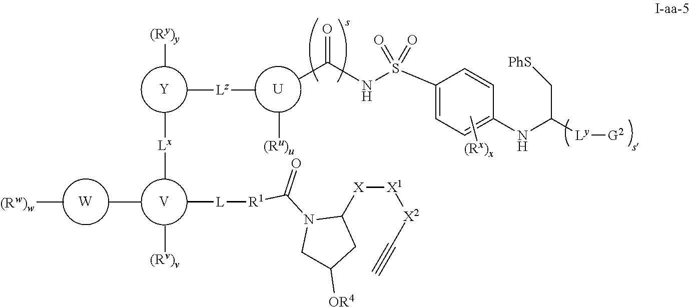

or a pharmaceutically acceptable salt thereof, wherein each of L, L^{x}, L^{y}, L^{z}, G², R¹, R⁴, R^{u}, R^{v}, R^{w}, R^{x}, R^{y}, Ring U, Ring V, Ring W, Ring Y, X, X¹, X², s, s′, u, v, w, x, and y is as defined above and described in embodiments herein, both singly and in combination.

In certain embodiments, the present invention provides a compound of formula I-aa-1, wherein R¹ is

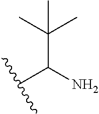 [SMILES: *C(N)C(C)(C)C]

(where one of the hydrogen atoms of the NH₂ group is replaced with -L-), s′, s″ and s′″ are 1, and G¹ is —SPh as shown, to provide a compound of formula I-aa-6:

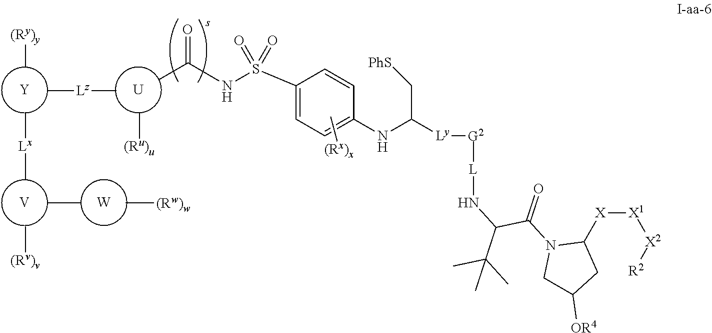

or a pharmaceutically acceptable salt thereof, wherein each of L, L^{x}, L^{y}, L^{z}, G², R², R⁴, R^{u}, R^{v}, R^{w}, R^{x}, R^{y}, Ring U, Ring V, Ring W, Ring Y, X, X¹, X², s, s′, u, v, w, x, and y is as defined above and described in embodiments herein, both singly and in combination.

In certain embodiments, the present invention provides a compound of formula I-aa-1, wherein R¹ is

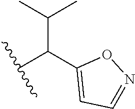 [SMILES: *C(c1ccno1)C(C)C]

(where one of the hydrogen atoms of the isoxazolyl group is replaced with -L-), s′, s″ and s′″ are 1, and G¹ is —SPh as shown, to provide a compound of formula I-aa-7:

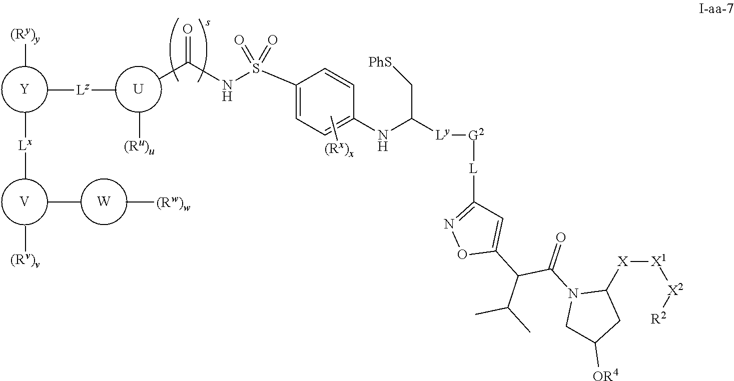

or a pharmaceutically acceptable salt thereof, wherein each of L, L^{x}, L^{y}, L^{z}, G², R², R⁴, R^{u}, R^{v}, R^{w}, R^{x}, R^{y}, Ring U, Ring V, Ring W, Ring Y, X, X¹, X², s, s′, u, v, w, x, and y is as defined above and described in embodiments herein, both singly and in combination.

In certain embodiments, the present invention provides a compound of formula I-aa-1, wherein R¹ is

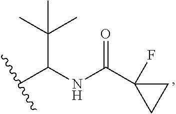 [SMILES: *C(NC(=O)C1(F)CC1)C(C)(C)C]

X² is phenylenyl, s′, s″ and s′″ are 1, and G¹ is —SPh as shown, to provide a compound of formula I-aa-8:

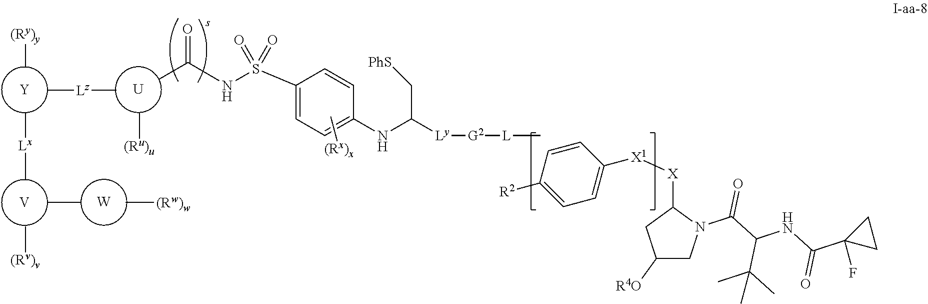

or a pharmaceutically acceptable salt thereof, wherein each of L, L^{x}, L^{y}, L^{z}, G², R², R⁴, R^{u}, R^{v}, R^{w}, R^{x}, R^{y}, Ring U, Ring V, Ring W, Ring Y, X, X¹, s, s′, u, v, w, x, and y is as defined above and described in embodiments herein, both singly and in combination.

In certain embodiments, the present invention provides a compound of formula I-aa-1, wherein R¹ is

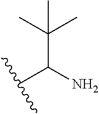 [SMILES: *C(N)C(C)(C)C]

(where one of the hydrogen atoms of the NH₂ group is replaced with -L-), s″ and s′″ are 1, and G¹ is —SPh as shown, to provide a compound of formula I-aa-9:

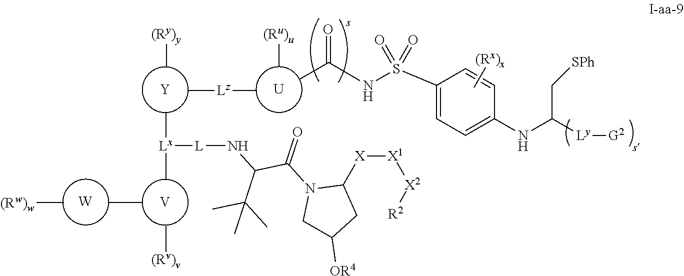

or a pharmaceutically acceptable salt thereof, wherein each of L, L^{x}, L^{y}, L^{z}, G², R², R⁴, R^{u}, R^{v}, R^{w}, R^{x}, R^{y}, Ring U, Ring V, Ring W, Ring Y, X, X¹, X², s, s′, u, v, w, x, and y is as defined above and described in embodiments herein, both singly and in combination.

In certain embodiments, the present invention provides a compound of formula I-bb-1:

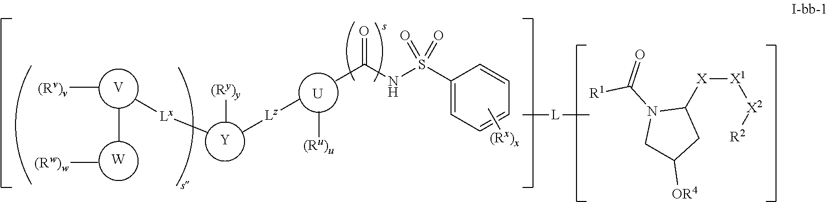

or a pharmaceutically acceptable salt, wherein:
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Ring W is a ring selected from phenyl, naphthyl, a 5-10 membered heteroaryl containing 1-4 heteroatoms independently selected from nitrogen, oxygen, selenium, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 5-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{u}, R^{v}, R^{w}, R^{x}, and R^{y} are, independently, hydrogen, halogen, C₁₋₆alkyl, C₁₋₆haloalkyl, —CN, —NO₂, —OR, —SR, —N(R)₂, —Si(R)₃, —S(O)₂R, —S(O)₂N(R)₂, —S(O)₂NRC(O)R—S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, —NRS(O)₂R, or R^{A};
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} and L^{z} are, independently, a covalent bond or a bivalent, saturated or partially unsaturated, straight or branched C₁₋₅ hydrocarbon chain, wherein 0-3 methylene units of L^{x} and L^{z} are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
s and s″ are, independently, 0 or 1;
u, v, w, x, and y are, independently, 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—

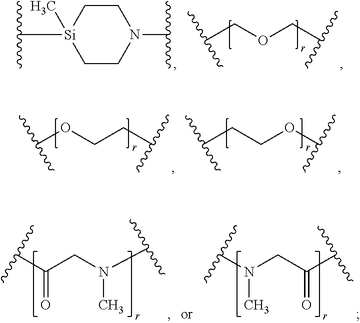

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R² is hydrogen, halogen, —CN,

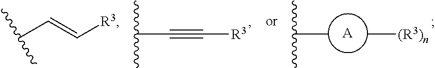

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, halogen, C₁₋₆alkyl, C₁₋₆haloalkyl, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, —N(R)SO₂R, or R^{A}; or
   two R³ groups are optionally taken together to form an optionally substituted 5-7 membered partially unsaturated or aryl fused ring having 0-2 heteroatoms independently selected from nitrogen, oxygen, and sulfur; and
R⁴ is hydrogen, —C(O)R, —C(O)OR, —C(O)NR₂, —P(O)R₂, —P(O)(OR)₂, —(CR₂)₁₋₃OP(O)R₂, —(CR₂)₁₋₃OP(O)(OR)₂, or R^{A};
n is 0, 1, 2, 4, or 5.

In certain embodiments, the present invention provides a compound of formula I-bb-1, wherein R¹ is

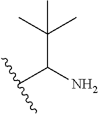 [SMILES: *C(N)C(C)(C)C]

(where one of the hydrogen atoms of the NH₂ group is replaced with -L-) and s″ is 1 as shown, to provide a compound of formula I-bb-2:

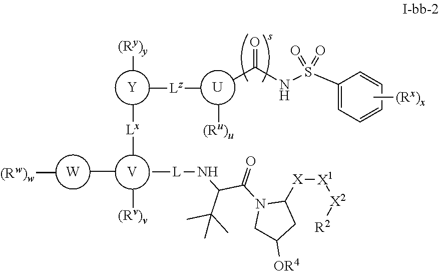

or a pharmaceutically acceptable salt thereof, wherein each of L, L^{x}, L^{z}, R², R⁴, R^{u}, R^{y}, R^{v}, R^{x}, R^{y}, Ring U, Ring V, Ring W, Ring Y, X, X¹, X², s, u, v, w, x, and y is as defined above and described in embodiments herein, both singly and in combination.

In certain embodiments, the present invention provides a compound of formula I-bb-1, wherein R¹ is

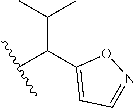 [SMILES: *C(c1ccno1)C(C)C]

(where one of the hydrogen atoms of the isoxazolyl group is replaced with -L-) and s″ is 1 as shown, to provide a compound of formula I-bb-3:

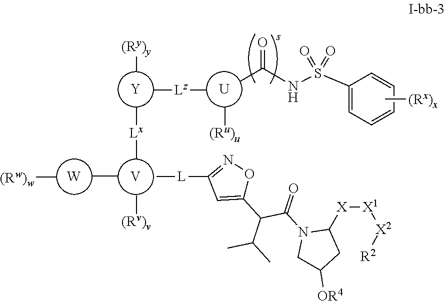

or a pharmaceutically acceptable salt thereof, wherein each of L, L^{x}, L^{z}, R², R⁴, R^{u}, R^{v}, R^{w}, R^{x}, R^{y}, Ring U, Ring V, Ring W, Ring Y, X, X¹, X², s, u, v, w, x, and y is as defined above and described in embodiments herein, both singly and in combination.

In certain embodiments, the present invention provides a compound of formula I-bb-1, wherein R¹ is

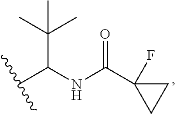 [SMILES: *C(NC(=O)C1(F)CC1)C(C)(C)C]

X² is phenylenyl, and s″ is 1 as shown, to provide a compound of formula I-bb-4:

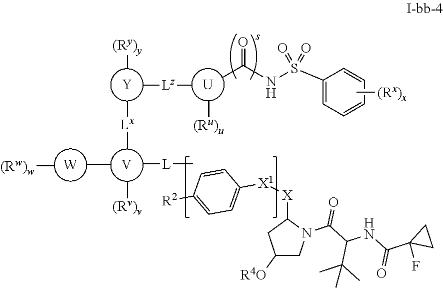

or a pharmaceutically acceptable salt thereof, wherein each of L, L^{x}, L^{z}, R², R⁴, R^{u}, R^{v}, R^{w}, R^{x}, R^{y}, Ring U, Ring V, Ring W, Ring Y, X, X¹, s, u, v, w, x, and y is as defined above and described in embodiments herein, both singly and in combination.

In certain embodiments, the present invention provides a compound of formula I-bb-1, wherein R² is

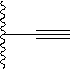 [SMILES: *C#C]

and s″ is 1 as shown, to provide a compound of formula I-bb-5:

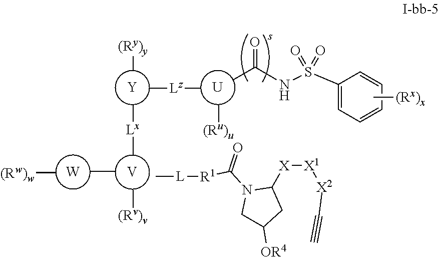

or a pharmaceutically acceptable salt thereof, wherein each of L, L^{x}, L^{z}, R¹, R⁴, R^{u}, R^{v}, R^{w}, R^{x}, R^{y}, Ring U, Ring V, Ring W, Ring Y, X, X¹, X², s, u, v, w, x, and y is as defined above and described in embodiments herein, both singly and in combination.

In certain embodiments, the present invention provides a compound of formula I-bb-1, wherein R¹ is

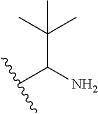 [SMILES: *C(N)C(C)(C)C]

(where one of the hydrogen atoms of the NH² group is replaced with -L-) and s″ is 1 as shown, to provide a compound of formula I-bb-6:

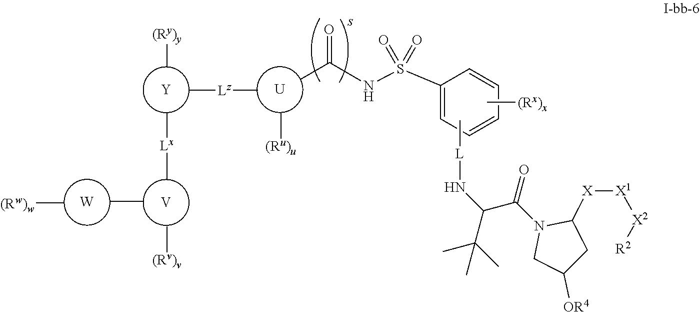

or a pharmaceutically acceptable salt thereof, wherein each of L, L^{x}, L^{z}, R², R⁴, R^{u}, R^{v}, R^{w}, R^{x}, R^{y}, Ring U, Ring V, Ring W, Ring Y, X, X¹, X², s, u, v, w, x, and y is as defined above and described in embodiments herein, both singly and in combination.

In certain embodiments, the present invention provides a compound of formula I-bb-1, wherein R¹ is

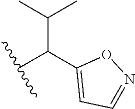 [SMILES: *C(c1ccno1)C(C)C]

(where one of the hydrogen atoms of the isoxazolyl group is replaced with -L-) and s″ is 1 as shown, to provide a compound of formula I-bb-7:

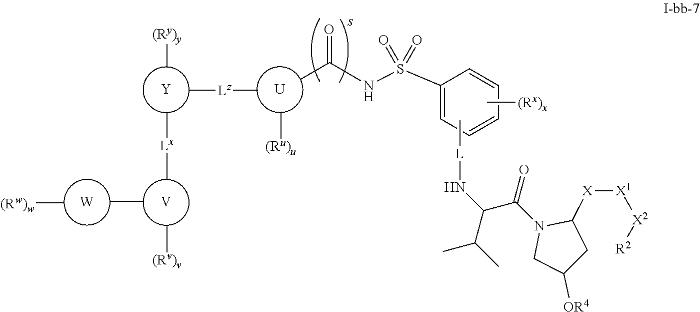

or a pharmaceutically acceptable salt thereof, wherein each of L, L^{x}, L^{z}, R², R⁴, R^{u}, R^{v}, R^{w}, R^{x}, R^{y}, Ring U, Ring V, Ring W, Ring Y, X, X¹, X², s, u, v, w, x, and y is as defined above and described in embodiments herein, both singly and in combination.

In certain embodiments, the present invention provides a compound of formula I-bb-1, wherein R¹ is

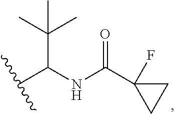 [SMILES: *C(NC(=O)C1(F)CC1)C(C)(C)C]

X² is phenylenyl, and s″ is 1 as shown, to provide a compound of formula I-bb-8:

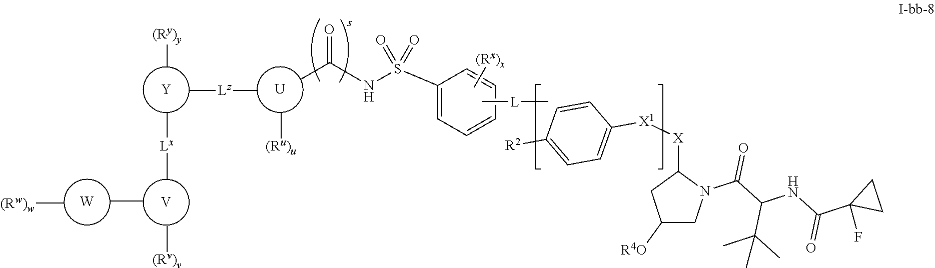

or a pharmaceutically acceptable salt thereof, wherein each of L, L^{x}, L^{z}, R², R⁴, R^{u}, R^{v}, R^{w}, R^{x}, R^{y}, Ring U, Ring V, Ring W, Ring Y, X, X¹, s, u, v, w, x, and y is as defined above and described in embodiments herein, both singly and in combination.

In certain embodiments, the present invention provides a compound of formula I-cc-1:

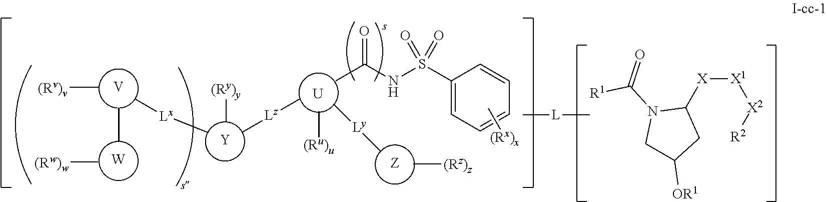

or a pharmaceutically acceptable salt, wherein:
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Ring W and Ring Z are, independently, a ring selected from phenyl, naphthyl, a 5-10 membered heteroaryl containing 1-4 heteroatoms independently selected from nitrogen, oxygen, selenium, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 5-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{u}, R^{v}, R^{w}, R^{x}, R^{y}, and R^{z} are, independently, hydrogen, halogen, C₁₋₆alkyl, C₁₋₆haloalkyl, —CN, —NO₂, —OR, —SR, —N(R)₂, —Si(R)₃, —S(O)₂R, —S(O)₂N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR, —NRC(O)R, —NRC(O)N(R)₂, —NP(O)(R)₂, —NRP(O)(OR)₂, —NRP(O)(OR)N(R)₂, —NRP(O)(N(R)₂)₂, —NRS(O)₂R, or R^{A};
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x}, L^{y}, and L^{z} are, independently, a covalent bond or a bivalent, saturated or partially unsaturated, straight or branched C₁₋₅ hydrocarbon chain, wherein 0-3 methylene units of L^{x}, L^{y}, and L^{z} are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
s and s″ are, independently, 0 or 1;
u, v, w, x, y, and z are, independently, 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—

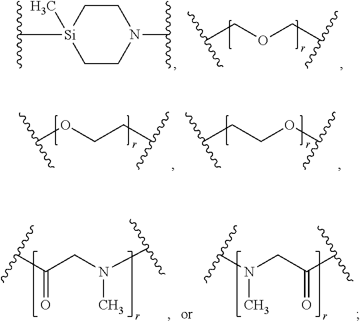

each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R² is hydrogen, halogen, —CN,

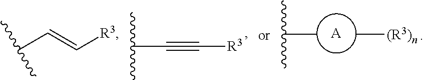

Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, halogen, C₁₋₆alkyl, C₁₋₆haloalkyl, —CN, —NO₂, —OR, —SR, —N(R)₂, —Si(R)₃, —SO₂R, —SON(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, —N(R)SO₂R, or R^{A}; or
   two R³ groups are optionally taken together to form an optionally substituted 5-7 membered partially unsaturated or aryl fused ring having 0-2 heteroatoms independently selected from nitrogen, oxygen, and sulfur; and
R⁴ is hydrogen, —C(O)R, —C(O)OR, —C(O)NR₂, —P(O)R₂, —P(O)(OR)₂, —(CR₂)₁₋₃OP(O)R₂, —(CR₂)₁₋₃OP(O)(OR)₂, or R^{A};
n is 0, 1, 2, 4, or 5.

In certain embodiments, the present invention provides a compound of formula I-cc-1, wherein R¹ is

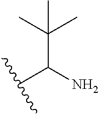 [SMILES: *C(N)C(C)(C)C]

(where one of the hydrogen atoms of the NH₂ group is replaced with -L-) and s″ is 1 as shown, to provide a compound of formula I-cc-2:

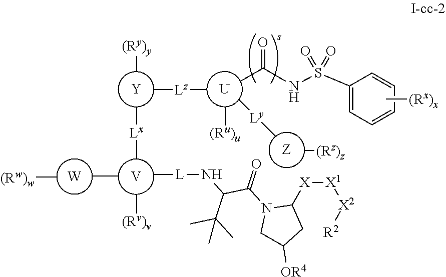

or a pharmaceutically acceptable salt thereof, wherein each of L, L^{x}, L^{y}, L^{z}, R², R⁴, R^{u}, R^{v}, R^{w}, R^{x}, R^{y}, Ring U, Ring V, Ring W, Ring Y, X, X¹, X², s, u, v, w, x, and y is as defined above and described in embodiments herein, both singly and in combination.

In certain embodiments, the present invention provides a compound of formula I-cc-1, wherein R¹ is

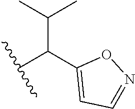 [SMILES: *C(c1ccno1)C(C)C]

(where one of the hydrogen atoms of the isoxazolyl group is replaced with -L-) and s″ is 1 as shown, to provide a compound of formula I-cc-3:

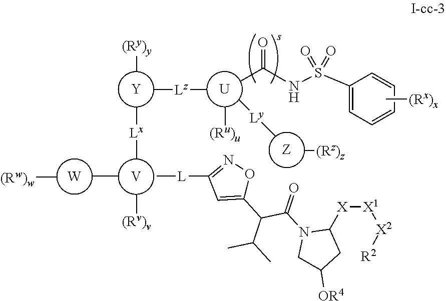

or a pharmaceutically acceptable salt thereof, wherein each of L, L^{x}, L^{y}, L^{z}, R², R⁴, R^{u}, R^{v}, R^{w}, R^{x}, R^{y}, Ring U, Ring V, Ring W, Ring Y, X, X¹, X², s, u, v, w, x, and y is as defined above and described in embodiments herein, both singly and in combination.

In certain embodiments, the present invention provides a compound of formula I-cc-1, wherein R¹ is

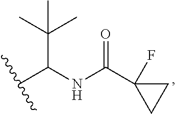 [SMILES: *C(NC(=O)C1(F)CC1)C(C)(C)C]

X² is phenylenyl, and s″ is 1 as shown, to provide a compound of formula I-cc-4:

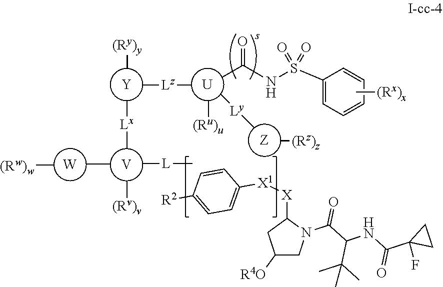

or a pharmaceutically acceptable salt thereof, wherein each of L, L^{x}, L^{y}, L^{z}, R², R⁴, R^{u}, R^{v}, R^{w}, R^{x}, R^{y}, Ring U, Ring V, Ring W, Ring Y, X, X¹, s, u, v, w, x, and y is as defined above and described in embodiments herein, both singly and in combination.

In certain embodiments, the present invention provides a compound of formula I-cc-1, wherein R² is

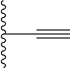 [SMILES: *C#C]

and s″ is 1 as shown, to provide a compound of formula I-cc-5:

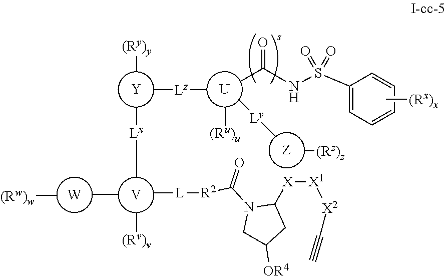

or a pharmaceutically acceptable salt thereof, wherein each of L, L^{x}, L^{y}, L^{z}, R², R⁴, R^{u}, R^{v}, R^{w}, R^{x}, R^{y}, Ring U, Ring V, Ring W, Ring Y, X, X¹, X², s, u, v, w, x, and y is as defined above and described in embodiments herein, both singly and in combination.

In certain embodiments, the present invention provides a compound of formula I-cc-1, wherein R¹ is

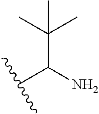 [SMILES: *C(N)C(C)(C)C]

(where one of the hydrogen atoms of the NH₂ group is replaced with -L-) and s″ is 1 as shown, to provide a compound of formula I-cc-6:

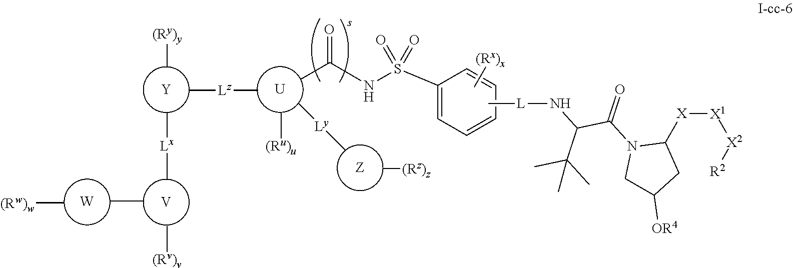

or a pharmaceutically acceptable salt thereof, wherein each of L, L^{x}, L^{y}, L^{z}, R², R⁴, R^{u}, R^{v}, R^{w}, R^{x}, R^{y}, Ring U, Ring V, Ring W, Ring Y, X, X¹, X², s, u, v, w, x, and y is as defined above and described in embodiments herein, both singly and in combination.

In certain embodiments, the present invention provides a compound of formula I-cc-1, wherein R¹ is

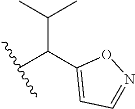 [SMILES: *C(c1ccno1)C(C)C]

(where one of the hydrogen atoms of the isoxazolyl group is replaced with -L-) and s″ is 1 as shown, to provide a compound of formula I-cc-7:

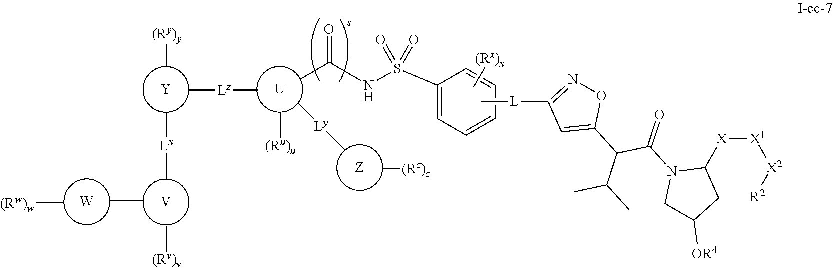

or a pharmaceutically acceptable salt thereof, wherein each of L, L^{x}, L^{y}, L^{z}, R², R⁴, R^{u}, R^{v}, R^{w}, R^{x}, R^{y}, Ring U, Ring V, Ring W, Ring Y, X, X¹, X², s, u, v, w, x, and y is as defined above and described in embodiments herein, both singly and in combination.

In certain embodiments, the present invention provides a compound of formula I-cc-1, wherein R¹ is

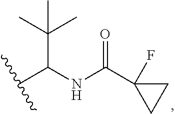 [SMILES: *C(NC(=O)C1(F)CC1)C(C)(C)C]

X² is phenylenyl, and s″ is 1 as shown, to provide a compound of formula I-cc-8:

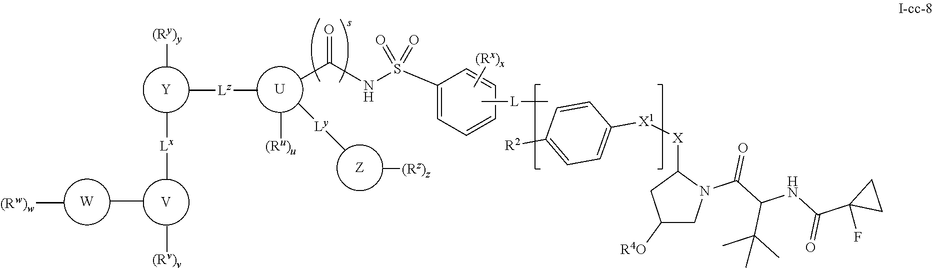

or a pharmaceutically acceptable salt thereof, wherein each of L, L^{x}, L^{y}, L^{z}, R², R⁴, R^{u}, R^{v}, R^{w}, R^{x}, R^{y}, Ring U, Ring V, Ring W, Ring Y, X, X¹, s, u, v, w, x, and y is as defined above and described in embodiments herein, both singly and in combination.

In some embodiments, LBM is

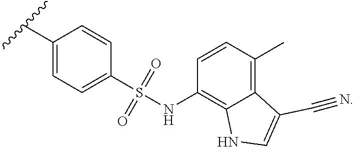 [SMILES: *c1ccc(S(=O)(=O)Nc2ccc(C)c3c(C#N)c[nH]c23)cc1]

In some embodiments, LBM is

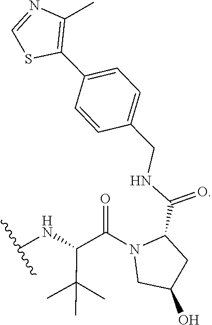 [SMILES: *N[C@H](C(=O)N1C[C@H](O)C[C@H]1C(=O)NCc1ccc(-c2scnc2C)cc1)C(C)(C)C]

In some embodiments, LBM is

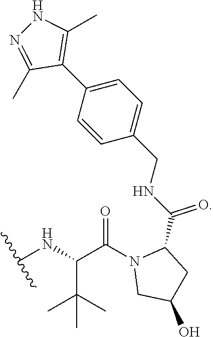 [SMILES: *N[C@H](C(=O)N1C[C@H](O)C[C@H]1C(=O)NCc1ccc(-c2c(C)n[nH]c2C)cc1)C(C)(C)C]

In some embodiments, LBM is

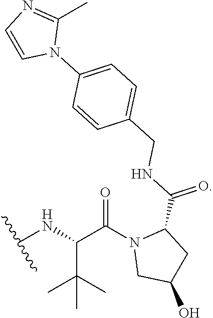 [SMILES: *N[C@H](C(=O)N1C[C@H](O)C[C@H]1C(=O)NCc1ccc(-n2ccnc2C)cc1)C(C)(C)C]

In some embodiments, LBM is

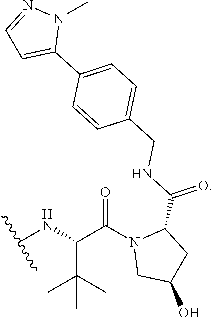 [SMILES: *N[C@H](C(=O)N1C[C@H](O)C[C@H]1C(=O)NCc1ccc(-c2ccnn2C)cc1)C(C)(C)C]

In some embodiments, LBM is

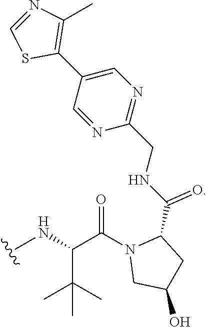

In some embodiments, LBM is

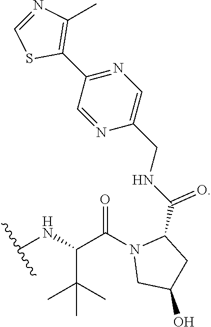 [SMILES: *N[C@H](C(=O)N1C[C@H](O)C[C@H]1C(=O)NCc1cnc(-c2scnc2C)cn1)C(C)(C)C]

In some embodiments, LBM is

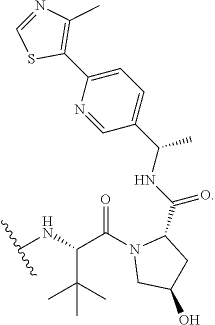 [SMILES: *N[C@H](C(=O)N1C[C@H](O)C[C@H]1C(=O)N[C@@H](C)c1ccc(-c2scnc2C)nc1)C(C)(C)C]

In some embodiments, LBM is

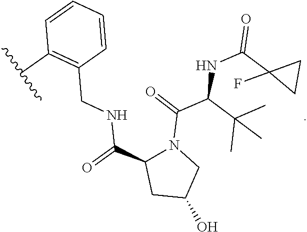 [SMILES: *c1ccccc1CNC(=O)[C@@H]1C[C@@H](O)CN1C(=O)[C@@H](NC(=O)C1(F)CC1)C(C)(C)C]

In some embodiments, LBM is

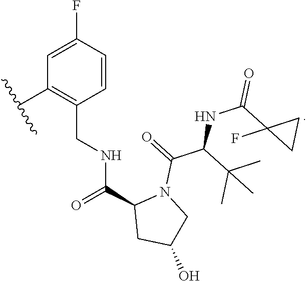 [SMILES: *c1cc(F)ccc1CNC(=O)[C@@H]1C[C@@H](O)CN1C(=O)[C@@H](NC(=O)C1(F)CC1)C(C)(C)C]

In some embodiments, LBM is

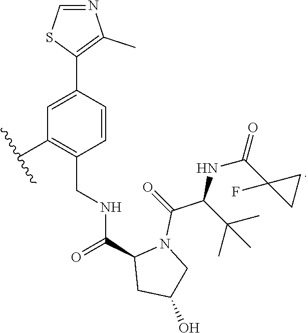 [SMILES: *c1cc(-c2scnc2C)ccc1CNC(=O)[C@@H]1C[C@@H](O)CN1C(=O)[C@@H](NC(=O)C1(F)CC1)C(C)(C)C]

In some embodiments, LBM is

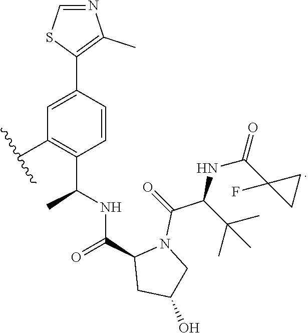 [SMILES: *c1cc(-c2scnc2C)ccc1[C@H](C)NC(=O)[C@@H]1C[C@@H](O)CN1C(=O)[C@@H](NC(=O)C1(F)CC1)C(C)(C)C]

In some embodiments, LBM is

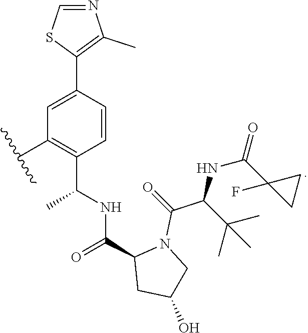 [SMILES: *c1cc(-c2scnc2C)ccc1[C@@H](C)NC(=O)[C@@H]1C[C@@H](O)CN1C(=O)[C@@H](NC(=O)C1(F)CC1)C(C)(C)C]

In some embodiments, LBM is

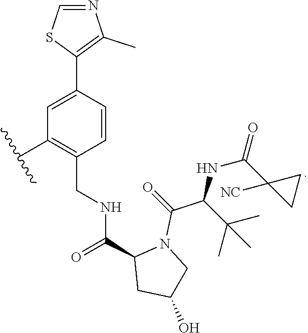 [SMILES: *c1cc(-c2scnc2C)ccc1CNC(=O)[C@@H]1C[C@@H](O)CN1C(=O)[C@@H](NC(=O)C1(C#N)CC1)C(C)(C)C]

In some embodiments, LBM is

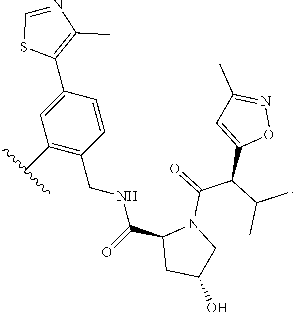 [SMILES: *c1cc(-c2scnc2C)ccc1CNC(=O)[C@@H]1C[C@@H](O)CN1C(=O)[C@@H](c1cc(C)no1)C(C)C]

In some embodiments, LBM is

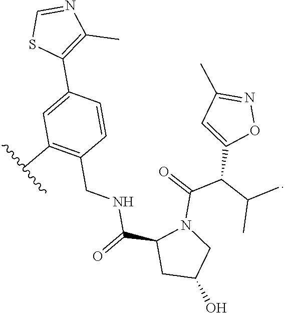 [SMILES: *c1cc(-c2scnc2C)ccc1CNC(=O)[C@@H]1C[C@@H](O)CN1C(=O)[C@H](c1cc(C)no1)C(C)C]

In some embodiments, LBM is

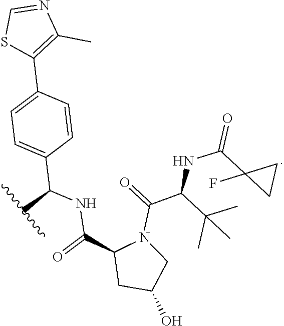 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, LBM is

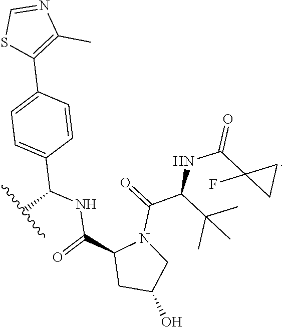 [SMILES: *[C@@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, LBM is

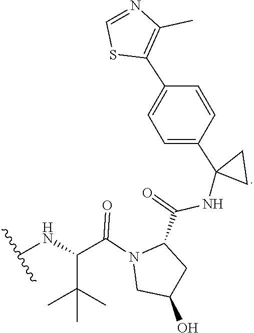 [SMILES: *N[C@H](C(=O)N1C[C@H](O)C[C@H]1C(=O)NC1(c2ccc(-c3scnc3C)cc2)CC1)C(C)(C)C]

In some embodiments, LBM is

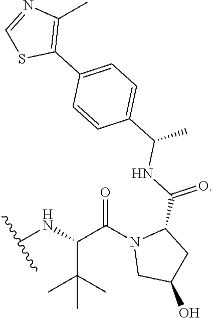 [SMILES: *N[C@H](C(=O)N1C[C@H](O)C[C@H]1C(=O)N[C@@H](C)c1ccc(-c2scnc2C)cc1)C(C)(C)C]

In some embodiments, LBM is

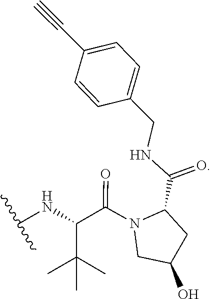 [SMILES: *N[C@H](C(=O)N1C[C@H](O)C[C@H]1C(=O)NCc1ccc(C#C)cc1)C(C)(C)C]

In some embodiments, LBM is

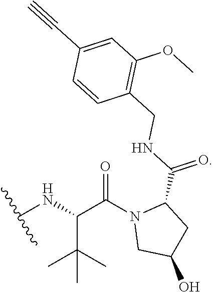 [SMILES: *N[C@H](C(=O)N1C[C@H](O)C[C@H]1C(=O)NCc1ccc(C#C)cc1OC)C(C)(C)C]

In some embodiments, LBM is

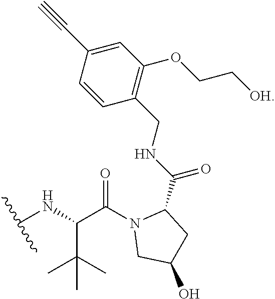 [SMILES: *N[C@H](C(=O)N1C[C@H](O)C[C@H]1C(=O)NCc1ccc(C#C)cc1OCCO)C(C)(C)C]

In some embodiments, LBM is

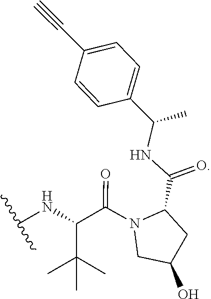 [SMILES: *N[C@H](C(=O)N1C[C@H](O)C[C@H]1C(=O)N[C@@H](C)c1ccc(C#C)cc1)C(C)(C)C]

In some embodiments, LBM is

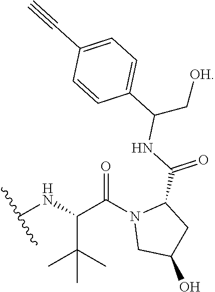 [SMILES: *N[C@H](C(=O)N1C[C@H](O)C[C@H]1C(=O)NC(CO)c1ccc(C#C)cc1)C(C)(C)C]

In some embodiments, LBM is

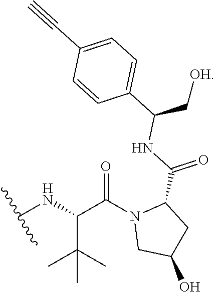 [SMILES: *N[C@H](C(=O)N1C[C@H](O)C[C@H]1C(=O)N[C@H](CO)c1ccc(C#C)cc1)C(C)(C)C]

In some embodiments, LBM is

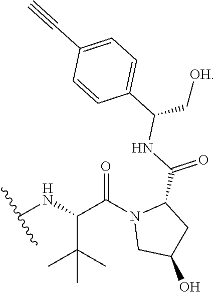 [SMILES: *N[C@H](C(=O)N1C[C@H](O)C[C@H]1C(=O)N[C@@H](CO)c1ccc(C#C)cc1)C(C)(C)C]

In some embodiments, LBM is

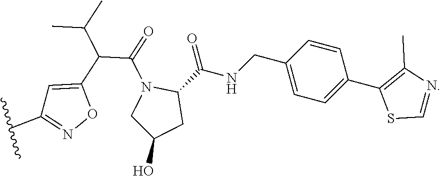 [SMILES: *c1cc(C(C(=O)N2C[C@H](O)C[C@H]2C(=O)NCc2ccc(-c3scnc3C)cc2)C(C)C)on1]

In some embodiments, LBM is

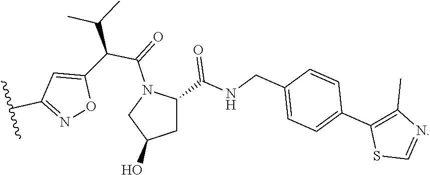 [SMILES: *c1cc([C@H](C(=O)N2C[C@H](O)C[C@H]2C(=O)NCc2ccc(-c3scnc3C)cc2)C(C)C)on1]

In some embodiments, LBM is

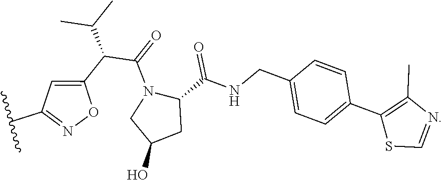 [SMILES: *c1cc([C@@H](C(=O)N2C[C@H](O)C[C@H]2C(=O)NCc2ccc(-c3scnc3C)cc2)C(C)C)on1]

In some embodiments, LBM is

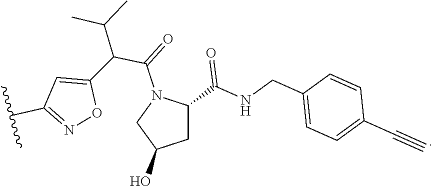 [SMILES: *c1cc(C(C(=O)N2C[C@H](O)C[C@H]2C(=O)NCc2ccc(C#C)cc2)C(C)C)on1]

In some embodiments, LBM is

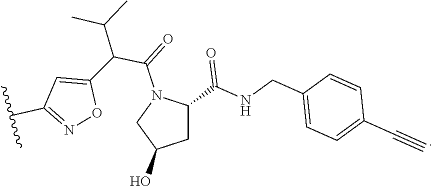 [SMILES: *c1cc(C(C(=O)N2C[C@H](O)C[C@H]2C(=O)NCc2ccc(C#C)cc2)C(C)C)on1]

In some embodiments, LBM is

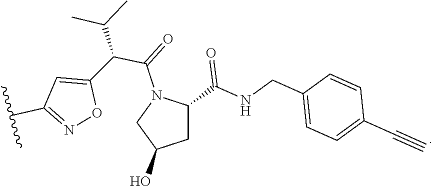 [SMILES: *c1cc([C@@H](C(=O)N2C[C@H](O)C[C@H]2C(=O)NCc2ccc(C#C)cc2)C(C)C)on1]

In some embodiments, LBM is

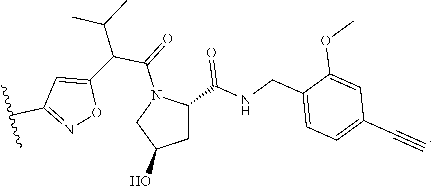 [SMILES: *c1cc(C(C(=O)N2C[C@H](O)C[C@H]2C(=O)NCc2ccc(C#C)cc2OC)C(C)C)on1]

In some embodiments, LBM is

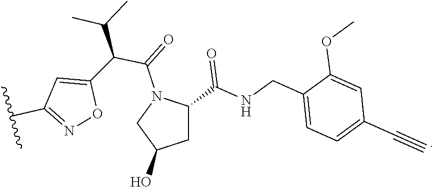 [SMILES: *c1cc([C@H](C(=O)N2C[C@H](O)C[C@H]2C(=O)NCc2ccc(C#C)cc2OC)C(C)C)on1]

In some embodiments, LBM is

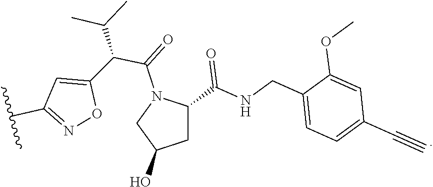 [SMILES: *c1cc([C@@H](C(=O)N2C[C@H](O)C[C@H]2C(=O)NCc2ccc(C#C)cc2OC)C(C)C)on1]

In some embodiments, LBM is

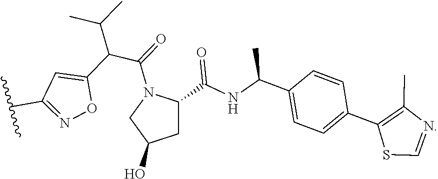 [SMILES: *c1cc(C(C(=O)N2C[C@H](O)C[C@H]2C(=O)N[C@@H](C)c2ccc(-c3scnc3C)cc2)C(C)C)on1]

In some embodiments, LBM is

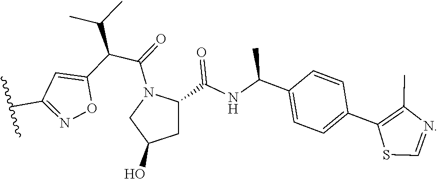 [SMILES: *c1cc([C@H](C(=O)N2C[C@H](O)C[C@H]2C(=O)N[C@@H](C)c2ccc(-c3scnc3C)cc2)C(C)C)on1]

In some embodiments, LBM is

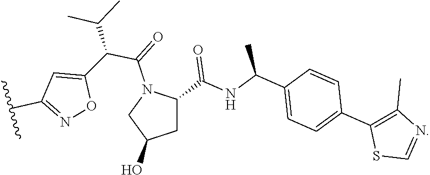 [SMILES: *c1cc([C@@H](C(=O)N2C[C@H](O)C[C@H]2C(=O)N[C@@H](C)c2ccc(-c3scnc3C)cc2)C(C)C)on1]

In some embodiments, LBM is

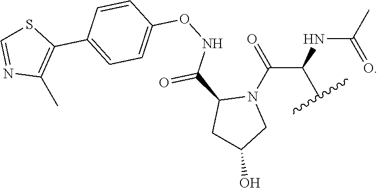 [SMILES: *[C@H](NC(C)=O)C(=O)N1C[C@H](O)C[C@H]1C(=O)NOc1ccc(-c2scnc2C)cc1]

In some embodiments, LBM is

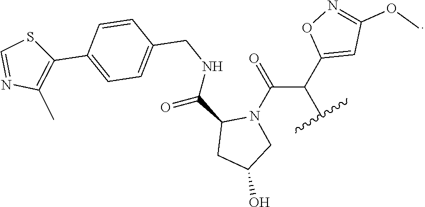 [SMILES: *C(C(=O)N1C[C@H](O)C[C@H]1C(=O)NCc1ccc(-c2scnc2C)cc1)c1cc(OC)no1]

In some embodiments, LBM is

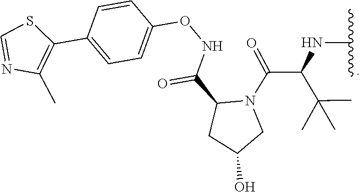 [SMILES: *N[C@H](C(=O)N1C[C@H](O)C[C@H]1C(=O)NOc1ccc(-c2scnc2C)cc1)C(C)(C)C]

In some embodiments, LBM is

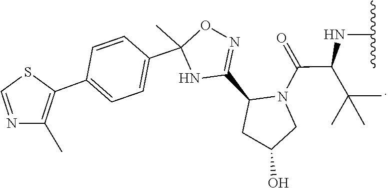 [SMILES: *N[C@H](C(=O)N1C[C@H](O)C[C@H]1C1=NOC(C)(c2ccc(-c3scnc3C)cc2)N1)C(C)(C)C]

In some embodiments, LBM is

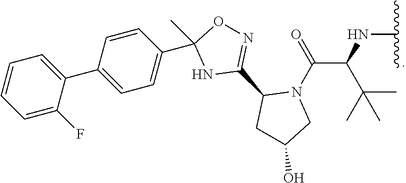 [SMILES: *N[C@H](C(=O)N1C[C@H](O)C[C@H]1C1=NOC(C)(c2ccc(-c3ccccc3F)cc2)N1)C(C)(C)C]

In some embodiments, LBM is

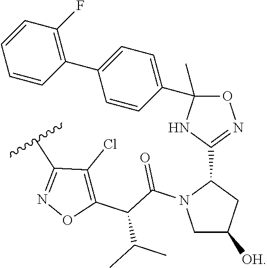 [SMILES: *c1noc([C@H](C(=O)N2C[C@H](O)C[C@H]2C2=NOC(C)(c3ccc(-c4ccccc4F)cc3)N2)C(C)C)c1Cl]

In some embodiments, LBM is

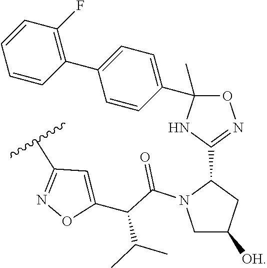 [SMILES: *c1cc([C@H](C(=O)N2C[C@H](O)C[C@H]2C2=NOC(C)(c3ccc(-c4ccccc4F)cc3)N2)C(C)C)on1]

In some embodiments, LBM is

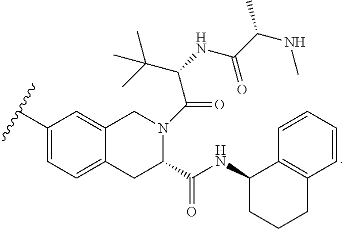 [SMILES: *c1ccc2c(c1)CN(C(=O)[C@@H](NC(=O)[C@H](C)NC)C(C)(C)C)[C@H](C(=O)N[C@@H]1CCCc3ccccc31)C2]

In some embodiments, LBM is

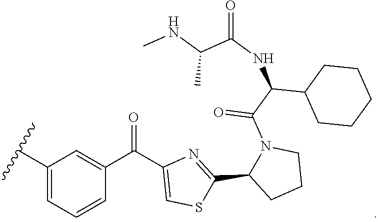 [SMILES: *c1cccc(C(=O)c2csc([C@@H]3CCCN3C(=O)[C@@H](NC(=O)[C@H](C)NC)C3CCCCC3)n2)c1]

In some embodiments, LBM is

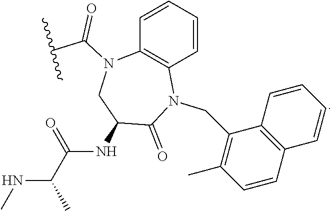 [SMILES: *C(=O)N1C[C@H](NC(=O)[C@H](C)NC)C(=O)N(Cc2c(C)ccc3ccccc23)c2ccccc21]

In some embodiments, LBM is

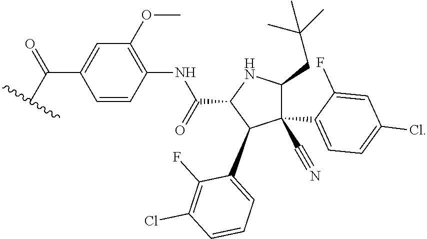 [SMILES: *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1]

In some embodiments, LBM is

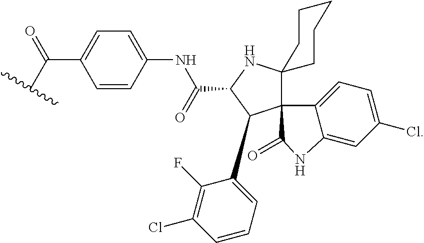 [SMILES: *C(=O)c1ccc(NC(=O)[C@@H]2NC3(CCCCC3)[C@@]3(C(=O)Nc4cc(Cl)ccc43)[C@H]2c2cccc(Cl)c2F)cc1]

In some embodiments, LBM is

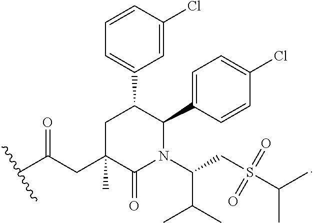 [SMILES: *C(=O)C[C@@]1(C)C[C@H](c2cccc(Cl)c2)[C@@H](c2ccc(Cl)cc2)N([C@H](CS(=O)(=O)C(C)C)C(C)C)C1=O]

In some embodiments, LBM is

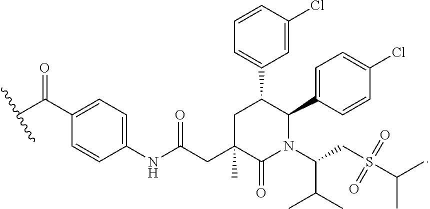 [SMILES: *C(=O)c1ccc(NC(=O)C[C@@]2(C)C[C@H](c3cccc(Cl)c3)[C@@H](c3ccc(Cl)cc3)N([C@H](CS(=O)(=O)C(C)C)C(C)C)C2=O)cc1]

In some embodiments, LBM is

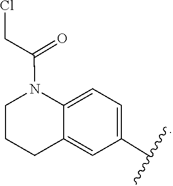 [SMILES: *c1ccc2c(c1)CCCN2C(=O)CCl]

In some embodiments, LBM is

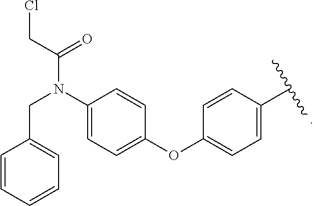 [SMILES: *c1ccc(Oc2ccc(N(Cc3ccccc3)C(=O)CCl)cc2)cc1]

In some embodiments, LBM is

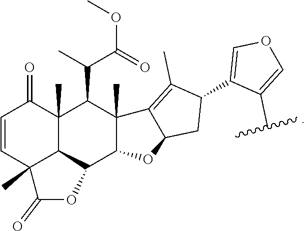 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45]

In some embodiments, LBM is

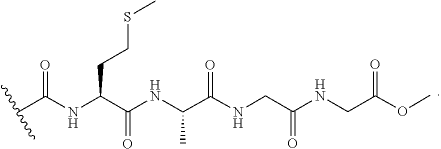 [SMILES: *C(=O)N[C@@H](CCSC)C(=O)N[C@@H](C)C(=O)NCC(=O)NCC(=O)OC]

In some embodiments, LBM is

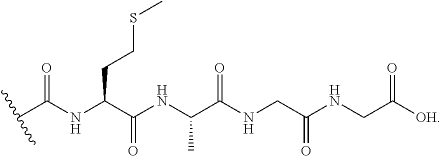 [SMILES: *C(=O)N[C@@H](CCSC)C(=O)N[C@@H](C)C(=O)NCC(=O)NCC(=O)O]

In some embodiments, LBM is

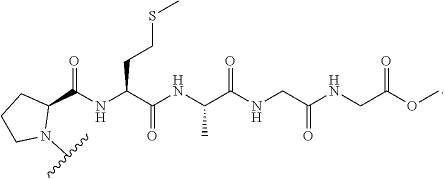 [SMILES: *N1CCC[C@H]1C(=O)N[C@@H](CCSC)C(=O)N[C@@H](C)C(=O)NCC(=O)NCC(=O)OC]

In some embodiments, LBM is

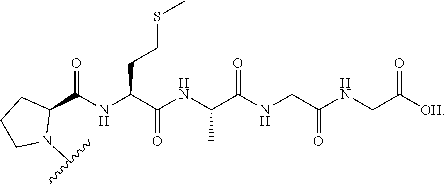 [SMILES: *N1CCC[C@H]1C(=O)N[C@@H](CCSC)C(=O)N[C@@H](C)C(=O)NCC(=O)NCC(=O)O]

In some embodiments, LBM is

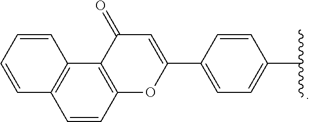 [SMILES: *c1ccc(-c2cc(=O)c3c(ccc4ccccc43)o2)cc1]

In some embodiments, LBM is

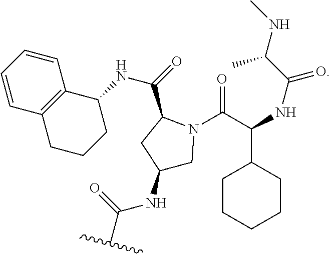 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1]

In some embodiments, LBM is

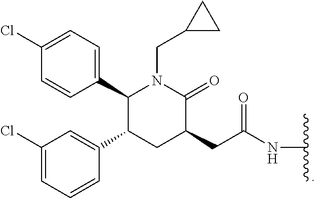 [SMILES: *NC(=O)C[C@H]1C[C@H](c2cccc(Cl)c2)[C@@H](c2ccc(Cl)cc2)N(CC2CC2)C1=O]

In some embodiments, LBM is

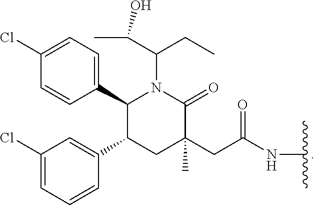 [SMILES: *NC(=O)C[C@@]1(C)C[C@H](c2cccc(Cl)c2)[C@@H](c2ccc(Cl)cc2)N(C(CC)[C@H](C)O)C1=O]

In some embodiments, LBM is

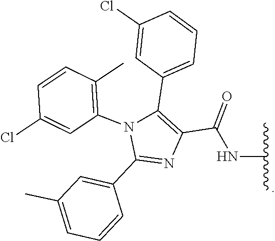 [SMILES: *NC(=O)c1nc(-c2cccc(C)c2)n(-c2cc(Cl)ccc2C)c1-c1cccc(Cl)c1]

In some embodiments, LBM is

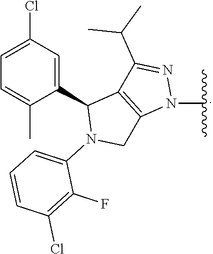 [SMILES: *n1nc(C(C)C)c2c1CN(c1cccc(Cl)c1F)[C@H]2c1cc(Cl)ccc1C]

In some embodiments, LBM is

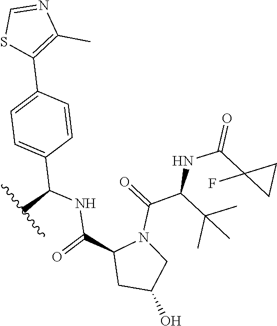 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1]

In some embodiments, LBM is

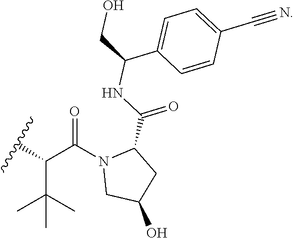 [SMILES: *[C@H](C(=O)N1C[C@H](O)C[C@H]1C(=O)N[C@@H](CO)c1ccc(C#N)cc1)C(C)(C)C]

In some embodiments, LBM is

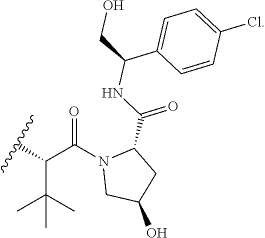 [SMILES: *[C@H](C(=O)N1C[C@H](O)C[C@H]1C(=O)N[C@@H](CO)c1ccc(Cl)cc1)C(C)(C)C]

In some embodiments, LBM is

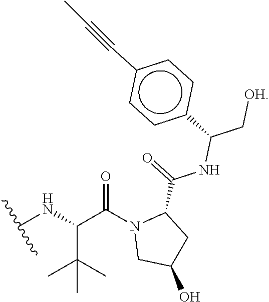

In some embodiments, LBM is

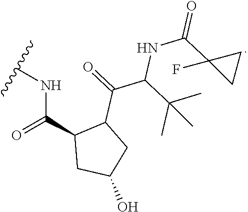 [SMILES: *NC(=O)[C@@H]1C[C@@H](O)CC1C(=O)C(NC(=O)C1(F)CC1)C(C)(C)C]

In some embodiments, LBM is

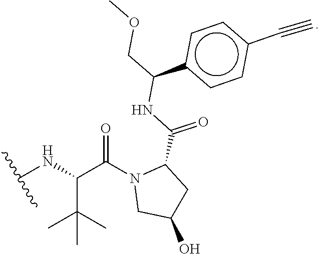

In some embodiments, LBM is

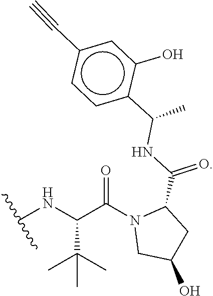

In some embodiments, LBM is

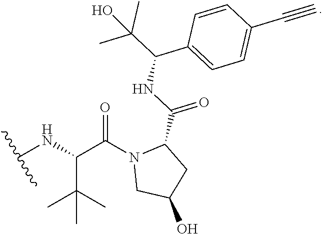 [SMILES: *N[C@H](C(=O)N1C[C@H](O)C[C@H]1C(=O)N[C@@H](c1ccc(C#C)cc1)C(C)(C)O)C(C)(C)C]

In some embodiments, LBM is

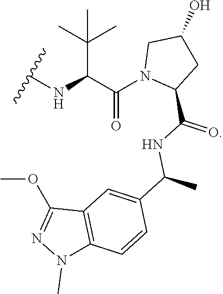 [SMILES: *N[C@H](C(=O)N1C[C@H](O)C[C@H]1C(=O)N[C@@H](C)c1ccc2c(c1)c(OC)nn2C)C(C)(C)C]

In certain embodiments, the present invention provides a compound of formula I, wherein LBM is a CRBN E3 ubiquitin ligase binding moiety thereby forming a compound of formula I-ll:

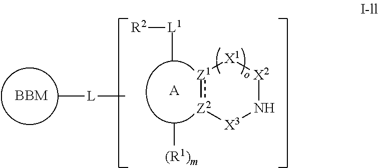

or a pharmaceutically acceptable salt thereof, wherein L and BBM are as defined above and described in embodiments herein, wherein:
each X¹ is independently —CH₂—, —O—, —NR—, —CF₂,

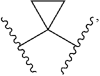 [SMILES: *C1(*)CC1]

### —C(O)—, —C(S)—, or

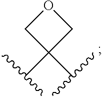 [SMILES: *C1(*)COC1]

X² and X¹ are independently —CH₂—, —C(O)—, —C(S)—, or

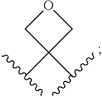 [SMILES: *C1(*)COC1]

Z¹ and Z² are independently a carbon atom or a nitrogen atom;
Ring A is a fused ring selected from benzo, a 4-6 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CR₂F, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
two R¹ groups are optionally taken together to form an optionally substituted 5-8 membered partially unsaturated or aryl fused ring having 0-2 heteroatoms independently selected from nitrogen, oxygen, and sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

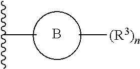

or hydrogen:
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
[symbol] is a single or double bond;
m is 0, 1, 2, 3 or 4;
n is 0, 1, 2, 3 or 4; and
is 0, 1, or 2.

As defined above and described herein X¹ is a covalent bond, —CH₂—, —O—, —NR—, —CF₂—,

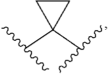 [SMILES: *C1(*)CC1]

### —C(O)—, —C(S)—, or

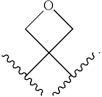 [SMILES: *C1(*)COC1]

In some embodiments, X¹ is a covalent bond. In some embodiments, X¹ is —CH₂—. In some embodiments, X¹ is —O—. In some embodiments, X¹ is —NR—. In some embodiments, X¹ is —CF₂—. In some embodiments, X¹ is

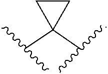 [SMILES: *C1(*)CC1]

In some embodiments, X¹ is —C(O)—. In some embodiments, X¹ is —C(S)—. In some embodiments, X¹ is

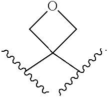 [SMILES: *C1(*)COC1]

In certain embodiments, X¹ is selected from those shown in the compounds of Table 1.

As defined above and described herein, X² and X³ are independently —CH₂—, —C(O)—, —C(S)—, or

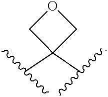 [SMILES: *C1(*)COC1]

In some embodiments, X² and X³ are independently —CH₂—. In some embodiments, X² and X³ are independently —C(O)—. In some embodiments, X² and X³ are independently —C(S)—. In some embodiments, X² and X³ are independently

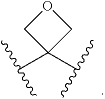 [SMILES: *C1(*)COC1]

In certain embodiments, X² and X³ are independently selected from those shown in the compounds of Table 1.

As defined above and described herein, X⁴ is a covalent bond, —CH₂—, —CR₂—, —O—, —NR—, —CF₂—,

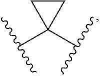 [SMILES: *C1(*)CC1]

### —C(O)—, —C(S)—, or

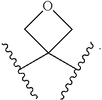 [SMILES: *C1(*)COC1]

In some embodiments, X⁴ is a covalent bond. In some embodiments, X⁴ is —CH₂—. In some embodiments, X⁴ is —CR₂—. In some embodiments, X⁴ is —O—. In some embodiments, X⁴ is —NR—. In some embodiments, X⁴ is —CF₂—. In some embodiments, X⁴ is

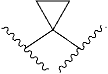 [SMILES: *C1(*)CC1]

In some embodiments, X⁴ is —C(O)—. In some embodiments, X⁴ is —C(S)—. In some embodiments, X⁴ is

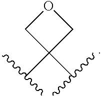 [SMILES: *C1(*)COC1]

In certain embodiments, X⁴ is selected from those shown in the compounds of Table 1.

As define above and described herein, Z¹ and Z² are independently a carbon atom or a nitrogen atom.

In some embodiments, Z¹ and Z² are independently a carbon atom. In some embodiments, Z¹ and Z² are independently a carbon atom.

In certain embodiments, Z¹ and Z² are independently selected from those shown in the compounds of Table 1.

As defined above and described herein, Ring A is fused ring selected from benzo or a 5-6 membered heteroaryl ring having 1-4 heteroatoms independently selected from nitrogen, oxygen, and sulfur.

In some embodiments, Ring A is benzo. In some embodiments, Ring A is a 5-6 membered heteroaryl ring having 1-4 heteroatoms independently selected from nitrogen, oxygen, and sulfur.

In some embodiments, Ring A is

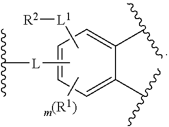

In certain embodiments, Ring A is selected from those shown in the compounds of Table 1.

In some embodiments, Ring C is a spiro-fused ring selected from a 4-10 membered saturated or partially unsaturated mono- or bicyclic carbocyclic or heterocyclic ring having 1-3 heteroatoms independently selected from nitrogen, oxygen, and sulfur. In some embodiments, Ring C is optionally further substituted with 1-2 oxo groups.

In certain embodiments, Ring C is selected from those shown in the compounds of Table 1.

As defined above and described herein, L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is a covalent bond. In some embodiments, L¹ is a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is —C(O)—.

In certain embodiments, L¹ is selected from those shown in the compounds of Table 1.

As defined above and described herein, each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃, or two R¹ groups are optionally taken together to form an optionally substituted 5-8 membered partially unsaturated or aryl fused ring having 0-2 heteroatoms independently selected from nitrogen, oxygen, and sulfur.

In some embodiments, R¹ is hydrogen. In some embodiments, R¹ is deuterium. In some embodiments, R¹ is R⁴. In some embodiments, R¹ is halogen. In some embodiments, R¹ is —CN. In some embodiments, R¹ is —NO₂. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —CF₂R. In some embodiments, R¹ is —CF₃. In some embodiments, R¹ is —CR₂(OR). In some embodiments, R¹ is —CR₂(NR₂). In some embodiments, R¹ is —C(O)R. In some embodiments, R¹ is —C(O)OR. In some embodiments, R¹ is —C(O)NR₂. In some embodiments, R¹ is —C(O)N(R)OR. In some embodiments, R¹ is —OC(O)R. In some embodiments, R¹ is —OC(O)NR₂. In some embodiments, R¹ is —C(S)NR₂. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)S(O)₂R. In some embodiments, R¹ is —OP(O)R₂. In some embodiments, R¹ is —OP(O)(OR)₂. In some embodiments, R¹ is —OP(O)(OR)NR₂. In some embodiments, R¹ is —OP(O)(NR₂)₂. In some embodiments, R¹ is —Si(OR)R₂. In some embodiments, R¹ is —SiR₃. In some embodiments, two R¹ groups are optionally taken together to form an optionally substituted 5-8 membered partially unsaturated or aryl fused ring having 0-2 heteroatoms independently selected from nitrogen, oxygen, and sulfur.

In some embodiments, R¹ is fluoro. In some embodiments, R¹ is

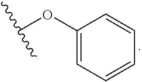

In certain embodiments, each R¹ is independently selected from those shown in the compounds of Table 1.

As defined above and described here, each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

As defined above and described herein, R² is selected from

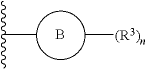

or hydrogen.

In some embodiment R² is

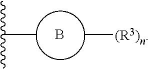

In some embodiments, R² is hydrogen.

In certain embodiments, R² is selected from those shown in the compounds of Table 1.

As defined above and described herein, Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups.

In some embodiments, Ring B is phenyl. In some embodiments, Ring B is a 4-10 membered saturated or partially unsaturated mono- or bicyclic carbocyclic or heterocyclic ring having 1-3 heteroatoms independently selected from nitrogen, oxygen, and sulfur In some embodiments, Ring B is a 5-6 membered heteroaryl ring having 1-4 heteroatoms independently selected from nitrogen, oxygen, and sulfur. In some embodiments, Ring B is further optionally substituted with 1-2 oxo groups.

In certain embodiments, Ring Bis selected from those shown in the compounds of Table 1.

As defined above and described herein, each R³ is independently selected from hydrogen, deuterium, R⁴, halogen, —CN, —NO₂, —OR, —SR, —NR₂, —S(O)₂R, —S(O)₃NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃.

In some embodiments, R³ is hydrogen. In some embodiments, R³ is deuterium. In some embodiments, R³ is R⁴. In some embodiments, R³ is halogen. In some embodiments, R³ is —CN. In some embodiments, R³ is —NO₂. In some embodiments, R³ is —OR. In some embodiments, R³ is —SR. In some embodiments, R³ is —NR₂. In some embodiments, R³ is —S(O)₂R. In some embodiments, R³ is —S(O)₂NR₂. In some embodiments, R³ is —S(O)R. In some embodiments, R³ is —CF₂R. In some embodiments, R³ is —CF₃. In some embodiments, R³ is —CR₂(OR). In some embodiments, R³ is —CR₂(NR₂). In some embodiments, R³ is —C(O)R. In some embodiments, R³ is —C(O)OR. In some embodiments, R³ is —C(O)NR₂. In some embodiments, R³ is —C(O)N(R)OR. In some embodiments, R³ is —OC(O)R. In some embodiments, R³ is —OC(O)NR₂. In some embodiments, R³ is —N(R)C(O)OR. In some embodiments, R³ is —N(R)C(O)R. In some embodiments, R³ is —N(R)C(O)NR₂. In some embodiments, R³ is —N(R)S(O)²R. In some embodiments, R³ is —OP(O)R₂. In some embodiments, R³ is —OP(O)(OR)₂. In some embodiments, R³ is —OP(O)(OR)NR₂. In some embodiments, R³ is —OP(O)(NR₂)₂. In some embodiments, R³ is —SiR₃.

In certain embodiments, R³ is selected from those shown in the compounds of Table 1.

As defined above and described herein, each R⁴ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁴ is an optionally substituted C₁₋₆ aliphatic. In some embodiments, R⁴ is an optionally substituted phenyl. In some embodiments, R⁴ is an optionally substituted 4-7 membered saturated or partially unsaturated heterocyclic ring having 1-2 heteroatoms independently selected from nitrogen, oxygen, and sulfur. In some embodiments, R⁴ is an optionally substituted 5-6 membered heteroaryl ring having 1-4 heteroatoms independently selected from nitrogen, oxygen, and sulfur.

In certain embodiments, R⁴ is selected from those shown in the compounds of Table 1.

As defined above and described herein, [symbol] is a single or double bond.

In some embodiments. [symbol] is a single bond. In some embodiments, [symbol] is a double bond.

In certain embodiments [symbol] is selected from those shown in the compounds of Table 1.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In certain embodiments, m is selected from those shown in the compounds of Table 1.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In certain embodiments, n is selected from those shown in the compounds of Table 1.

As defined above and described herein, o is 0, 1, or 2.

In some embodiments, n is 0. In some embodiments, n is 1. In some embodiments, m is 2.

In certain embodiments, o is selected from those shown in the compounds of Table 1.

In some embodiments, the present invention provides a compound of formula I-cc, wherein Ring A is benzo, o is 1, X¹ is —CH₂—, X² and X³ are —C(O)—, and Z¹ and Z² are carbon atoms as shown, to provide a compound of formula I-cc-1:

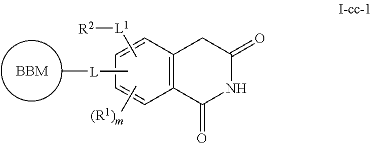

or a pharmaceutically acceptable salt thereof, wherein each of BBM, L, L¹, R¹, R², and m is as defined above and described in embodiments herein, both singly and in combination.

In some embodiments, the present invention provides a compound of formula I-cc, wherein Ring A is benzo, o is 1, X¹, X² and X³ are —C(O)—, and Z¹ and Z² are carbon atoms as shown, to provide a compound of formula I-cc-12:

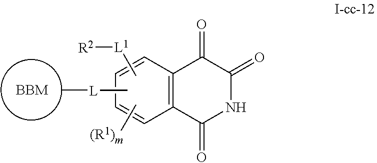

or a pharmaceutically acceptable salt thereof, wherein each of BBM, L, L¹, R¹, R², and m is as defined above and described in embodiments herein, both singly and in combination.

In some embodiments, LBM is

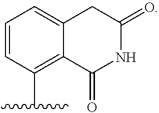 [SMILES: *c1cccc2c1C(=O)NC(=O)C2]

In some embodiments, LBM is

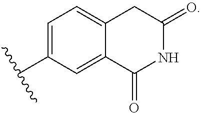 [SMILES: *c1ccc2c(c1)C(=O)NC(=O)C2]

In some embodiments, LBM is

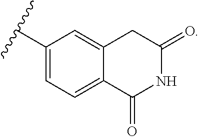 [SMILES: *c1ccc2c(c1)CC(=O)NC2=O]

In some embodiments, LBM is

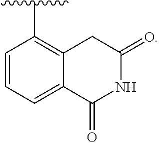 [SMILES: *c1cccc2c1CC(=O)NC2=O]

In some embodiments, LBM is

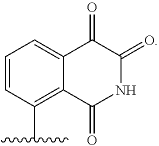 [SMILES: *c1cccc2c1C(=O)NC(=O)C2=O]

In some embodiments, LBM is

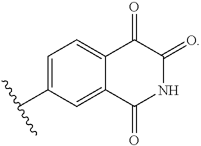 [SMILES: *c1ccc2c(c1)C(=O)NC(=O)C2=O]

In some embodiments, LBM is

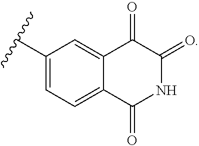 [SMILES: *c1ccc2c(c1)C(=O)C(=O)NC2=O]

In some embodiments, LBM is

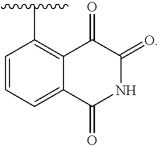 [SMILES: *c1cccc2c1C(=O)C(=O)NC2=O]

In some embodiments, LBM is selected from those in Table 1.

In certain embodiments, the present invention provides a compound of formula I, wherein LBM is a RPN13 binding moiety thereby forming a compound of formula I-o-1:

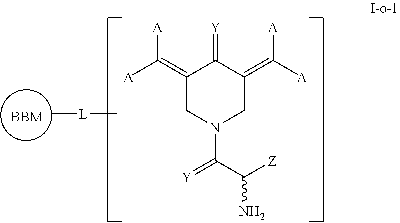

or a pharmaceutically acceptable salt thereof, wherein L and BBM are as defined above and described in embodiments herein, and wherein each of the variables A, Y, and Z is as described and defined in WO 2019/165229, the entirety of each of which is herein incorporated by reference.

In certain embodiments, the present invention provides a compound of formula I, wherein LBM is a Ubr1 binding moiety as described in Shanmugasundaram, K. et al, J. Bio. Chem. 2019, doi: 10.1074/jbc.AC119.010790, the entirety of each of which is herein incorporated by reference, thereby forming a compound of formula I-o-2 or I-o-3:

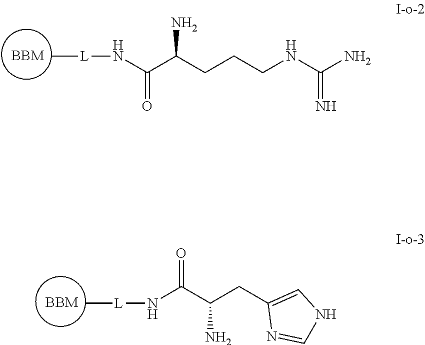

or a pharmaceutically acceptable salt thereof, wherein L and BBM are as defined above and described in embodiments herein.

In certain embodiments, the present invention provides a compound of formula I, wherein LBM is a cereblon binding moiety thereby forming a compound of formula I-o-4:

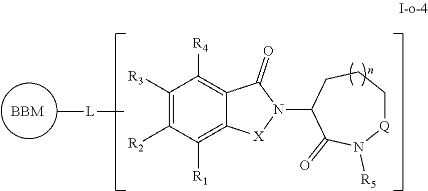

or a pharmaceutically acceptable salt thereof, wherein L and BBM are as defined above and described in embodiments herein, and wherein each of the variables R₁, R₂, R₃, R₄, R₅, Q, X, and n is as described and defined in US 2019/276474, the entirety of each of which is herein incorporated by reference.

In certain embodiments, the present invention provides a compound of formula I, wherein LBM is a cereblon E3 ubiquitin ligase binding moiety thereby forming a compound of formula I-o-5, I-o-6, I-o-7 or I-o-8:

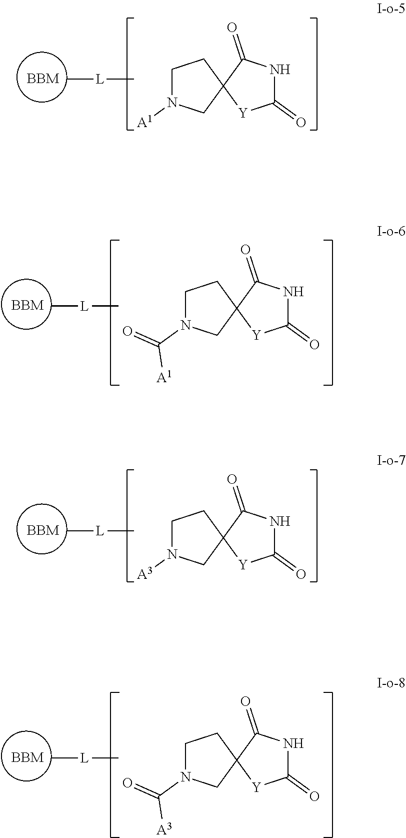

or a pharmaceutically acceptable salt thereof, wherein L and BBM are as defined above and described in embodiments herein, and wherein each of the variables Y, A¹, and A³ is as described and defined in WO 2019/236483, the entirety of each of which is herein incorporated by reference.

In certain embodiments, the present invention provides a compound of formula I, wherein LBM is human kelch-like ECH-associated protein 1 (KEAP1) of formula I-o-9:

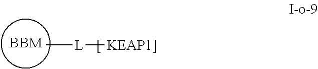

or a pharmaceutically acceptable salt thereof.

In certain embodiments, the present invention provides a compound of formula I, wherein LBM is KEAP1 binding moiety as recited in Lu et al., Euro. J. Med. Chem., 2018, 146:251-9, thereby forming a compound of formula I-o-10:

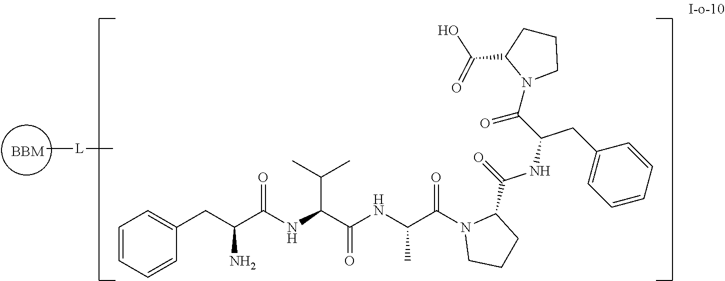

or a pharmaceutically acceptable salt thereof, wherein L and BBM are as defined above and described in embodiments herein.

In certain embodiments, the present invention provides a compound of formula I, wherein LBM is KEAP1-NRF2 binding moiety thereby forming a compound of formula I-o-11 or I-o-12:

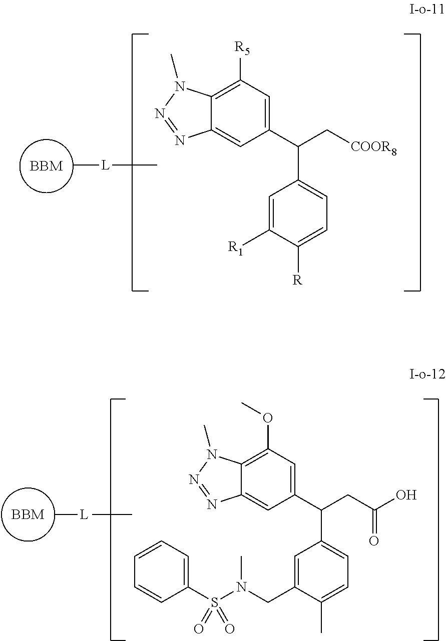

or a pharmaceutically acceptable salt thereof, wherein L and BBM are as defined above and described in embodiments herein, wherein each of the variables R, R₁, R₅, and R₈ is as described and defined in WO 2020/018788, the entirety of each of which is herein incorporated by reference.

In certain embodiments, the present invention provides a compound of formula I, wherein LBM is KEAP1-NRF2 binding moiety as recited in Tong et al., “Targeted Protein Degradation via a Covalent Reversible Degrader Based on Bardoxolone”, ChemRxiv 2020, thereby forming a compound of formula I-o-13 or I-o-14:

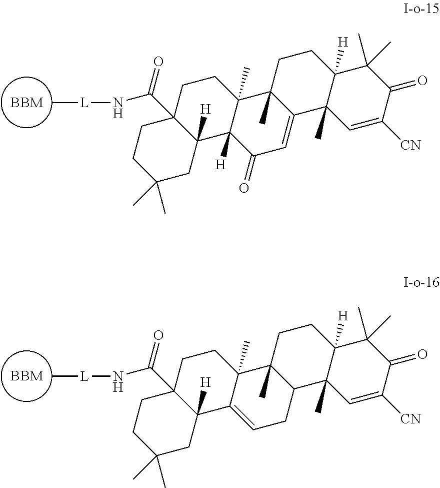

or a pharmaceutically acceptable salt thereof, wherein L and BBM are as defined above and described in embodiments herein.

### DCAF1 Binding Moiety (DBM)

In some embodiments, DIM is DBM.

In some embodiments, DBM is a DCAF1 binding moiety.

In certain embodiments, the present invention provides a compound of formula I, wherein DBM is a DCAF1 binding moiety of formula I-s:

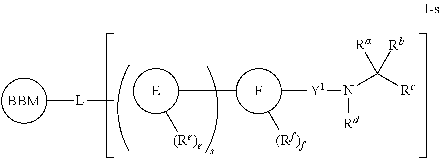

or a pharmaceutically acceptable salt thereof, wherein L and BBM are as defined and described herein, and wherein:
Ring E is phenyl, a 4-7 membered partially unsaturated carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring F is phenylenyl, a 4-10 membered partially unsaturated carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Y¹ is a C₁₋₃ hydrocarbon chain wherein each methylene is optionally substituted with —CR₂—, —CR(OR)—, —C(O)—, —C(NR)—, —C(NOR)—, —S(O)—, or —S(O)₂—;
R^{a} is an optionally substituted C₁₋₆ aliphatic or

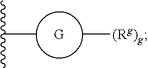

Ring G is phenyl, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur;
R^{b} is hydrogen, an optionally substituted C₁₋₆ aliphatic, phenyl, or a 5-6 membered heteroaryl with 1-4 heteroatoms independently selected from nitrogen, oxygen and sulfur, or:
   R^{a} and R^{b} are optionally taken together with their intervening atoms to form an optionally substituted 9-10 membered saturated or partially unsaturated bicyclic ring having 0-3 heteroatoms independently selected from nitrogen, oxygen, and sulfur, or:
   when Y¹ is —C(NR)—, R^{b} is optionally taken together with R of —C(NR)— with their intervening atoms to form a 5-7 membered partially unsaturated heterocyclyl with 0-1 heteroatoms, in addition to the 2 nitrogen atoms within the heterocyclyl, independently selected from nitrogen, oxygen, and sulfur;
R^{c} is —CR₂CONR₂, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur;
R^{d} is hydrogen, or:
   when R^{c} is —CR₂CONR₂, R^{d} is optionally taken together with a single R of —CR₂CONR₂ with their intervening atoms to form a 5-7 membered saturated or partially unsaturated heterocyclyl with 0-3 heteroatoms, in addition to the nitrogen atom to which R^{d} is attached, independently selected from nitrogen, oxygen, and sulfur:
R^{e}, R^{f}, and R^{g} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-7 membered saturated or partially unsaturated ring having 0-3 heteroatoms, in addition to the atom to which they are attached, independently selected from nitrogen, oxygen, and sulfur;
s is 0 or 1; and
each of e, f, and g are independently 0, 1, 2, 3, or 4;
wherein DBM is further optionally substituted with

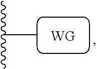

wherein

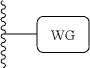

is a warhead group.

In certain embodiments, the present invention provides a compound of formula I, wherein DBM is a DCAF1 binding moiety of formula I-t:

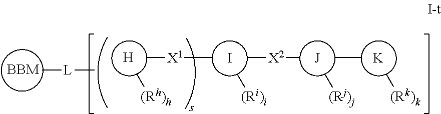

or a pharmaceutically acceptable salt thereof, wherein L and BBM are as defined and described herein, and wherein:
Ring H is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
Ring I is phenylenyl, a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring J is a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
Ring K is phenyl, naphthyl, a 9-10 membered saturated or partially unsaturated bicyclic heterocyclylenyl with 1-3 heteroatoms independently selected from nitrogen, oxygen, and sulfur, or a 5-13 membered monocyclic, bicyclic, or tricyclic heteroarylenyl with 1-5 heteroatoms independently selected from nitrogen, oxygen and sulfur;
R^{h}, Rⁱ, R^{j}, and R^{k} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂, or:
   an Rⁱ group on Ring I and an R^{j} group or Ring J are optionally taken together with their intervening atoms to form a 5-8 membered saturated or partially unsaturated ring having 0-3 heteroatoms independently selected from nitrogen, oxygen, and sulfur:
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-7 membered saturated or partially unsaturated ring having 0-3 heteroatoms, in addition to the atom to which they are attached, independently selected from nitrogen, oxygen, and sulfur:
each of X¹ and X² is independently a covalent bond, spiro-fusion between the two rings that X¹ or X² connect, —CR₂—, —CR(OR)—, —CRF—, —CF₂—, —NR—, —O—, —S—, or —S(O)₂—;
s is 0 or 1; and
each of w, x, y, and z are independently 0, 1, 2, 3, or 4;
wherein DBM is further optionally substituted with

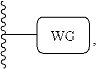

wherein

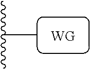

is a warhead group.

As described above and defined herein, Ring E is phenyl, a 4-7 membered partially unsaturated carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur.

In some embodiments, Ring E is phenyl. In some embodiments, Ring E is a 4-7 membered partially unsaturated carbocyclyl. In some embodiments, Ring E is a 4-7 membered partially unsaturated heterocyclyl with 1-3 heteroatoms independently selected from nitrogen, oxygen, and sulfur. In some embodiments, Ring E is a 5-9 membered monocyclic or bicyclic heteroarylenyl with 1-4 heteroatoms independently selected from nitrogen, oxygen and sulfur.

In some embodiments, Ring E is cyclobutyl, azetinyl, cyclohexyl, cyclohexenyl, tetrahydro-2H-pyranyl, pyrrolidinyl, 4,5-dihydro-1H-pyrazolyl, piperidinyl, phenyl, isoxazolyl, isothiazolyl, pyrazolyl, pyridyl, pyridazinyl, pyrimidinyl, indolyl, benzoimidazolyl, pyrazolo[1,5-a]pyridyl, or [1,2,4]triazolo[1,5-a]pyridyl.

In some embodiments, Ring E is as depicted in the compounds of Table 3, below.

As described above and defined herein, Ring F is phenylenyl, a 4-10 membered partially unsaturated carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur.

In some embodiments, Ring F is phenylenyl. In some embodiments, Ring F is a 4-10 membered partially unsaturated carbocyclylenyl. In some embodiments, Ring F is a 4-10 membered partially unsaturated heterocyclylenyl with 1-3 heteroatoms independently selected from nitrogen, oxygen, and sulfur. In some embodiments, Ring F is a 5-9 membered monocyclic or bicyclic heteroarylenyl with 1-4 heteroatoms independently selected from nitrogen, oxygen and sulfur.

In some embodiments, Ring F is cyclobutylenyl, azetinylenyl, cyclopentylenyl cyclohexyl, phenylenyl, pyrrolylenyl, imidazolylenyl, pyrazolylenyl, 1,2,3-triazolylenyl, 1,2,4-triazolylenyl, pyridylenyl, indazolyl, 4,5,6,7-tetrahydro-1H-pyrazolo[4,3-b]pyridyl, benzoimidazolyl, 3,4-dihydroquinolinyl, or 4,5,6,7-tetrahydro-1H-pyrazolo[4,3-c]pyridyl.

In some embodiments, Ring F is as depicted in the compounds of Table 3, below.

As described above and defined herein, Ring G is phenyl, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur.

In some embodiments, Ring G is phenyl. In some embodiments, Ring G is a 5-7 membered saturated or partially unsaturated carbocyclyl. In some embodiments, Ring G is a 5-7 membered saturated or partially unsaturated heterocyclyl with 1-3 heteroatoms independently selected from nitrogen, oxygen, and sulfur. In some embodiments, Ring G is a 5-6 membered heteroaryl with 1-4 heteroatoms independently selected from nitrogen, oxygen and sulfur.

In some embodiments, Ring G is cyclohexyl, cyclohexenyl, isothiazolyl, phenyl, or pyridyl.

In some embodiments, Ring G is as depicted in the compounds of Table 3, below.

As described above and defined herein, Ring H is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur.

In some embodiments, Ring H is a 3-11 membered saturated or partially unsaturated monocyclic, bicyclic, bridged bicyclic, or spirocyclic carbocyclyl. In some embodiments, Ring His a 3-11 membered saturated or partially unsaturated monocyclic, bicyclic, bridged bicyclic, or spirocyclic heterocyclyl with 1-3 heteroatoms independently selected from nitrogen, oxygen, and sulfur.

In some embodiments, Ring H is cyclopropyl, cyclobutyl, azetinyl, pyrrolidinyl, cyclohexyl, piperidinyl, piperazinyl, 3,6-dihydro-2H-pyranyl, tetrahydro-2H-pyranyl, morpholinyl, piperzinyl, 2,7-diazaspiro[3,5]nonanyl, 3,4-dihydro-2H-pyrido[3,2-b][1,4]oxazinyl, 2-oxa-5-azabicyclo[2.2.1]heptanyl, 6-oxa-3-azabicyclo[3.1.1]heptanyl, or 2-oxa-5-azabicyclo[2.2.2]octanyl.

In some embodiments, Ring H is as depicted in the compounds of Table 3, below.

As described above and defined herein, Ring I is phenylenyl, a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur.

In some embodiments, Ring I is phenylenyl. In some embodiments, Ring I is a 3-11 membered saturated or partially unsaturated monocyclic, bicyclic, bridged bicyclic, or spirocyclic carbocyclylenyl. In some embodiments, Ring I is a 3-11 membered saturated or partially unsaturated monocyclic, bicyclic, bridged bicyclic, or spirocyclic heterocyclylenyl with 1-3 heteroatoms independently selected from nitrogen, oxygen, and sulfur. In some embodiments, Ring I is a 5-9 membered monocyclic or bicyclic heteroarylenyl with 1-4 heteroatoms independently selected from nitrogen, oxygen and sulfur.

In some embodiments, Ring I is phenylenyl, imidazolylenyl, pyrazolylenyl, oxazolylenyl, thiazolylenyl, 1,2-thiazinanylenyl, pyridylenyl, pyridazinylenyl, pyrimidinylenyl, 2,6-diazaspiro[3.5]nonanylenyl, 2,3-dihydro-1H-pyrrolo[2,3-b]pyridylenyl, 2,3-dihydro-1H-pyrrolo[3,2-c]pyridylenyl, 1H-pyrrolo[2,3-b]pyridylenyl, 3H-imidazo[4,5-b]pyridylenyl, 9H-purinylenyl, 1,2,3,4-tetrahydro-1,8-naphthyridinylenyl, or 1,2,3,4-tetrahydro-1,6-naphthyridinylenyl.

In some embodiments, Ring I is as depicted in the compounds of Table 3, below.

As described above and defined herein, Ring J is a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur.

In some embodiments, Ring J is a 3-11 membered saturated or partially unsaturated monocyclic, bicyclic, bridged bicyclic, or spirocyclic carbocyclyl. In some embodiments, Ring J is a 3-11 membered saturated or partially unsaturated monocyclic, bicyclic, bridged bicyclic, or spirocyclic heterocyclyl with 1-3 heteroatoms independently selected from nitrogen, oxygen, and sulfur.

In some embodiments, Ring J is cyclohexylenyl, azetidinylenyl, pyrrolidinylenyl, imidazolylenyl, piperidinylenyl, piperzinylenyl, azepanylenyl, 8-azabicyclo[3.2.1]octanylenyl, 2-azabicyclo[3.2.1]octanylenyl, 2-azabicyclo[3.2.2]nonanylenyl, octahydro-1H-pyrrolo[3,2-b]pyridylenyl, decahydro-1,5-naphthyridinylenyl, 9-azabicyclo[3.3.1]nonanylenyl, 5-azaspiro[3.5]nonanylenyl, 2-oxa-5-azaspiro[3.5]nonanylenyl, or 2,6-diazaspiro[3.5]nonanylenyl.

In some embodiments, Ring J is as depicted in the compounds of Table 3, below.

As described above and defined herein, Ring K is phenyl, naphthyl, a 9-10 membered saturated or partially unsaturated bicyclic heterocyclylenyl with 1-3 heteroatoms independently selected from nitrogen, oxygen, and sulfur, or a 5-13 membered monocyclic, bicyclic, or tricyclic heteroarylenyl with 1-5 heteroatoms independently selected from nitrogen, oxygen and sulfur.

In some embodiments, Ring K is phenyl. In some embodiments, Ring K is naphthyl. In some embodiments, Ring K is a 9-10 membered saturated or partially unsaturated bicyclic heterocyclylenyl with 1-3 heteroatoms independently selected from nitrogen, oxygen, and sulfur. In some embodiments, Ring K is a 5-13 membered monocyclic, bicyclic, or tricyclic heteroarylenyl with 1-5 heteroatoms independently selected from nitrogen, oxygen and sulfur.

In some embodiments, Ring K is 1,2,3-triazolyl, thiazolyl, pyrazolyl, phenyl, pyridyl, pyridazinyl, pyrimidinyl, indazolyl, benzo[d]isoxazolyl, benzo[d]isothiazolyl, pyrazolo[1,5-a]pyrimidinyl, 2,3-dihydro-1H-pyrrolo[2,3-c]pyridinyl, 6,7-dihydro-5H-cyclopenta[b]pyridinyl, 2,3-dihydro-1H-pyrrolo[3,2-c]pyridinyl, naphthyl, quinolinyl, isoquinolinyl, 1,6-naphthyridinyl, phthalazinyl, quinazolinyl, 2,7-naphthyridinyl, or tetrazolo[1,5-a]quinoxalinyl.

In some embodiments, Ring K is as depicted in the compounds of Table 3, below.

As described above and defined herein, R^{a} is an optionally substituted C₁₋₆ aliphatic or

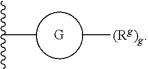

In some embodiments, R^{a} is an optionally substituted C₁₋₆ aliphatic. In some embodiments, R^{a} is

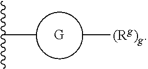

In some embodiments, Ring R^{a} is methyl.

In some embodiments, Ring R^{a} is as depicted in the compounds of Table 3, below.

As described above and defined herein, R^{b} is hydrogen, an optionally substituted C₁₋₆ aliphatic, phenyl, or a 5-6 membered heteroaryl with 1-4 heteroatoms independently selected from nitrogen, oxygen and sulfur, or R^{a} and R^{b} are optionally taken together with their intervening atoms to form an optionally substituted 9-10 membered saturated or partially unsaturated bicyclic ring having 0-3 heteroatoms independently selected from nitrogen, oxygen, and sulfur, or when Y is —C(NR)—, R^{b} is optionally taken together with R of —C(NR)— with their intervening atoms to form a 5-7 membered partially unsaturated heterocyclyl with 0-1 heteroatoms, in addition to the 2 nitrogen atoms within the heterocyclyl, independently selected from nitrogen, oxygen, and sulfur.

In some embodiments, R^{b} is hydrogen. In some embodiments, R^{b} is hydrogen is an optionally substituted C₁₋₆ aliphatic. In some embodiments, R^{b} is hydrogen is phenyl. In some embodiments, R^{b} is hydrogen is a 5-6 membered heteroaryl with 1-4 heteroatoms independently selected from nitrogen, oxygen and sulfur. In some embodiments, R^{a} and R^{b} are optionally taken together with their intervening atoms to form an optionally substituted 9-10 membered saturated or partially unsaturated bicyclic ring having 0-3 heteroatoms independently selected from nitrogen, oxygen, and sulfur. In some embodiments, when Y is —C(NR)—, R^{b} is optionally taken together with R of —C(NR)— with their intervening atoms to form a 5-7 membered partially unsaturated heterocyclyl with 0-1 heteroatoms, in addition to the 2 nitrogen atoms within the heterocyclyl, independently selected from nitrogen, oxygen, and sulfur.

In some embodiment, R^{b} is methyl, cyclopropyl, phenyl, —CO₂H, —CH₂cyclopropyl, —CH₂OH, —CH₂OMe, or —CH₂CO₂H.

In some embodiments, Ring R^{b} is as depicted in the compounds of Table 3, below.

As described above and defined herein, R^{c} is —CR₂CONR₂, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur.

In some embodiments, R^{c} is —CR₂CONR₂. In some embodiments, R^{c} is a 5-7 membered saturated or partially unsaturated carbocyclyl. In some embodiments, R^{c} is a 5-7 membered saturated or partially unsaturated heterocyclyl with 1-3 heteroatoms independently selected from nitrogen, oxygen, and sulfur. In some embodiments, R^{c} is a 5-6 membered heteroaryl with 1-4 heteroatoms independently selected from nitrogen, oxygen and sulfur.

In some embodiments, R^{c} is —CH₂CONH₂, —CH(Me)CONH₂, —CH₂CONHMe, —CH₂CONHEt, —CH₂CONHCH₂Ph, —CH₂CONHcyclopropyl, pyrrolidin-2-onyl, piperidin-2-only, or isoxazolyl.

In some embodiments, Ring R^{c} is as depicted in the compounds of Table 3, below.

As described above and defined herein, R^{d} is hydrogen, or when R^{c} is —CR₂CONR₂, R^{d} is optionally taken together with a single R of —CR₂CONR₂ with their intervening atoms to form a 5-7 membered saturated or partially unsaturated heterocyclyl with 0-3 heteroatoms, in addition to the nitrogen atom to which R^{d} is attached, independently selected from nitrogen, oxygen, and sulfur.

In some embodiments, R^{d} is hydrogen.

In some embodiments, Ring R^{d} is as depicted in the compounds of Table 3, below.

As described above and defined herein, R^{e}, R^{f}, R^{g}, R^{h}, Rⁱ, R^{j}, and R^{k} are each independently selected from hydrogen, OXO, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂, or an Rⁱ group on Ring I and an R^{j} group or Ring J are optionally taken together with their intervening atoms to form a 5-8 membered saturated or partially unsaturated ring having 0-3 heteroatoms independently selected from nitrogen, oxygen, and sulfur.

In some embodiments, one or more of R^{e}, R^{f}, R^{g}, R^{h}, Rⁱ, R^{j}, and R^{k} is hydrogen. In some embodiments, one or more of R^{e}, R^{f}, R^{g}, R^{h}, Rⁱ, R^{j}, and R^{k} is oxo. In some embodiments, one or more of R^{e}, R^{f}, R^{g}, R^{h}, Rⁱ, R^{j}, and R^{k} is R^{A}. In some embodiments, one or more of R^{e}, R^{f}, R^{g}, R^{h}, Rⁱ, R^{j}, and R^{k} is halogen. In some embodiments, one or more of R^{e}, R^{f}, R^{g}, R^{h}, Rⁱ, R^{j}, and R^{k} is —CN. In some embodiments, one or more of R^{e}, R^{f}, R^{g}, R^{h}, Rⁱ, R^{j}, and R^{k} is —NO₂. In some embodiments, one or more of R^{e}, R^{f}, R^{g}, R^{h}, Rⁱ, R^{j}, and R^{k} is —OR. In some embodiments, one or more of R^{e}, R^{f}, R^{g}, R^{h}, Rⁱ, R^{j} and R^{k} is —SR. In some embodiments, one or more of R^{e}, R^{f}, R^{g}, R^{h}, Rⁱ, R^{j} and R^{k} is —NR₂. In some embodiments, one or more of R^{e}, R^{f}, R^{g}, R^{h}, Rⁱ, R^{j}, and R^{k} is —SiR₃. In some embodiments, one or more of R^{e}, R^{f}, R^{g}, R^{h}, Rⁱ, R^{j}, and R^{k} is —S(O)₂R. In some embodiments, one or more of R^{e}, R^{f}, R^{g}, R^{h}, Rⁱ, R^{j}, and R^{k} is —S(O)₂NR₂. In some embodiments, one or more of R^{e}, R^{f}, R^{g}, R^{h}, Rⁱ, R^{j}, and R^{k} is —S(O)R. In some embodiments, one or more of R^{e}, R^{f}, R^{g}, R^{h}, Rⁱ, R^{j}, and R^{k} is —C(O)R. In some embodiments, one or more of R^{e}, R^{f}, R^{g}, R^{h}, Rⁱ, R^{j} and R^{k} is —C(O)OR. In some embodiments, one or more of R^{e}, R^{f}, R^{g}, R^{h}, Rⁱ, R^{j}, and R^{k} is —C(O)NR₂. In some embodiments, one or more of R^{e}, R^{f}, R^{g}, R^{h}, Rⁱ, R^{j}, and R^{k} is —C(O)NROR. In some embodiments, one or more of R^{e}, R^{f}, R^{g}, R^{h}, Rⁱ, R^{j}, and R^{k} is —OC(O)R. In some embodiments, one or more of R^{e}, R^{f}, R^{g}, R^{h}, Rⁱ, R^{j}, and R^{k} is —OC(O)NR₂. In some embodiments, one or more of R^{e}, R^{f}, R^{g}, R^{h}, Rⁱ, R^{j}, and R^{k} is —OP(O)R₂. In some embodiments, one or more of R^{e}, R^{f}, R^{g}, R^{h}, Rⁱ, R^{j}, and R^{k} is —OP(O)(OR)₂. In some embodiments, one or more of R^{e}, R^{f}, R^{g}, R^{h}, Rⁱ, R^{j} and R^{k} is —OP(O)(OR)NR₂. In some embodiments, one or more of R^{e}, R^{f}, R^{g}, R^{h}, Rⁱ, R^{j} and R^{k} is —OP(O)(NR₂)₂. In some embodiments, one or more of R^{e}, R^{f}, R^{g}, R^{h}, Rⁱ, R^{j} and R^{k} is —NRC(O)OR. In some embodiments, one or more of R^{e}, R^{f}, R^{g}, R^{h}, Rⁱ, R^{j}, and R^{k} is —NRC(O)R. In some embodiments, one or more of R^{e}, R^{f}, R^{g}, R^{h}, Rⁱ, R^{j}, and R^{k} is —NRC(O)N(R)₂. In some embodiments, one or more of R^{e}·R^{f}, R^{g}, R^{h}, Rⁱ, R^{j}, and R^{k} is —NRS(O)₂R. In some embodiments, one or more of R^{e}, R^{f}, R^{g}, R^{h}, Rⁱ, R^{j}, and R^{k} is —NP(O)R₂. In some embodiments, one or more of R^{e}, R^{f}, R^{g}, R^{h}, Rⁱ, R^{j}, and R^{k} is —NRP(O)(OR)₂. In some embodiments, one or more of R^{e}, R^{f}, R^{g}, R^{h}, Rⁱ, R^{j}, and R^{k} is —NRP(O)(OR)NR₂. In some embodiments, one or more of R^{e}, R^{f}, R^{g}, R^{h}, Rⁱ, R^{j}, and R^{k} is —NRP(O)(NR₂)₂. In some embodiments, one or more of R^{e}, R^{f}, R^{g}, R^{h}, Rⁱ, R^{j}, and R^{k} is —P(O)R₂. In some embodiments, one or more of R^{e}, R^{f}, R^{g}, R^{h}, Rⁱ, R^{j}, and R^{g} is —P(O)(OR)₂. In some embodiments, one or more of R^{e}, R^{f}, R^{g}, R^{h}, Rⁱ, R^{j}, and R^{k} is —P(O)(OR)NR₂. In some embodiments, one or more of R^{e}, R^{f}, R^{g}, R^{h}, Rⁱ, R^{j}, and R^{k} is —P(O)(NR₂)₂. In some embodiments, an Rⁱ group on Ring I and an R^{j} group or Ring J are taken together with their intervening atoms to form a 5-8 membered saturated or partially unsaturated ring having 0-3 heteroatoms independently selected from nitrogen, oxygen, and sulfur.

In some embodiments, R^{g} is hydrogen, oxo, fluoro, chloro, —CN, methyl, —CO₂H, —CO₂Me, —CONH₂, —C(O)CHCH₂, —OH, —OMe, —CH₂CHF₂, —CH₂OMe, —CH₂CO₂H, —CH₂SO₂Me, —CH₂CH₂O₂H, —CH₂CH₂SO₂Me, —CH₂CH₂OMe, —NHC(O)CHCH₂, tetrazolyl, or N-methyltetrazolyl.

In some embodiments, R^{f} is hydrogen, oxo, methyl, isopropyl, —CH₂cyclopropyl, —CH₂cyclopentyl, —CH₂cyclohexyl, —CH morpholinyl, —CH₂Ph, —CH thiazolyl, —CH₂pyrimidinyl, —CH₂CH₂OMe, —CH₂CH₂Ph, —C(O)Me, —C(O)CHCH₂, —C(O) Ph, —C(O)pyrimidinyl, —NH₂, —NHC(O)CHCH₂, —CH₂NHC(O)CHCH₂, —CCNHC(O)CHCH₂, —NHcyclohexyl, —NHphenyl, or —NHpyrimidinyl,

In some embodiments, R^{h} is hydrogen, oxo, fluoro, methyl, ethyl, n-propyl, b-butyl, —CH₂CHOMe, —C(O)CHCH₂, —NHC(O)CHCH₂, —N(Me)C(O)CHCH₂, —CH₂NHC(O)CHCH₂, or

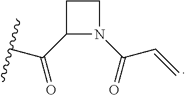 [SMILES: *C(=O)C1CCN1C(=O)C=C]

In some embodiments, R^{g} is hydrogen, oxo, fluoro, chloro, —CN, methyl, —CONH₂, —OH, or —OMe.

In some embodiments, Rⁱ is hydrogen, oxo, fluoro, chloro, methyl, —CF₃, —CH₂OH, —CN, —OH, —OMe, —NH₂, or —N(Me)CH₂CH₂CH₂N(Me)C(O)CHCH₂.

In some embodiments, R^{j} is hydrogen, oxo, fluoro, methyl, —CH₂F, —CH₂OH, —CO₂H, —C(O)NH₂, —OH, —OMe, or —S(O)₂NH₂.

In some embodiments, Rⁱ and R^{j}, are taken together by —CH₂CH₂— or —CH₂CH₂CH₂—.

In some embodiments, R^{k} is hydrogen, oxo, fluoro, chloro, —CN, methyl, isobutyl, —CF₃, —CH₂CF₃, —CH₂OH, —CH₂CO₂Me, —CH(OH)Me, —CH(NH₂)cyclopropyl, —CH₂Ph, —OH, —OMe, —OCF₃, —OiPr, OPh, —NHC(O)Me, —NHC(O)CHCH₂, —S(O)₂NH₂, 1,2,3-triazolyl, piperdinyl, N-methylpiperdinyl, phenyl, or pyridyl.

In some embodiments, R^{e}, R^{f}, R^{g}, R^{h}, Rⁱ, R^{j}, and R^{k} are as depicted in the compounds of Table 3, below.

As described above and defined herein, each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is an optionally substituted C₁₋₆ aliphatic. In some embodiments, R^{A} is an optionally substituted phenyl. In some embodiments, R^{A} is an optionally substituted 3-7 membered saturated or partially unsaturated carbocyclic. In some embodiments, R^{A} is an optionally substituted saturated or partially unsaturated heterocyclic ring having 1-2 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is C₁₋₆ alkyl (e.g., methyl, ethyl, isopropyl). In some embodiments, R^{A} is C₁₋₆ haloalkyl (e.g., —CF₃, —CHF₂).

In some embodiment, R^{A} is as depicted in the compounds of Table 3, below.

As described above and defined herein, each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atoms to form an optionally substituted 3-7 membered saturated or partially unsaturated ring having 0-3 heteroatoms, in addition to the atom to which they are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated carbocyclic. In some embodiments, R is an optionally substituted 4-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom are optionally taken together with their intervening atoms to form optionally substituted 3-7 membered saturated or partially unsaturated ring having 0-3 heteroatoms, in addition to the atom to which they are attached, independently selected from nitrogen, oxygen, and sulfur.

In some embodiment, R is as depicted in the compounds of Table 3, below.

As described above and defined herein, each of X¹ and X² is independently a is a covalent bond, spiro-fusion between the two rings that X¹ or X² connect, —CR₂—, —CR(OR)—, —CRF—, —CF₂—, —NR—, —O—, —S—, or —S(O)₂—.

In some embodiments, X¹ and/or X² is a covalent bond. In some embodiments, X¹ and/or X² is —CR₂—. In some embodiments, X¹ and/or X² is —CR(OR)—. In some embodiments, X¹ and/or X² is —CRF—. In some embodiments, X¹ and/or X² is —CF₂—. In some embodiments, X¹ and/or X² is —NR—. In some embodiments, X¹ and/or X² is —O—. In some embodiments, X¹ and/or X² is —S—. In some embodiments, X¹ and/or X² is —S(O)₂—. In some embodiments, X¹ and/or X² represents spiro-fusion between the two rings that X¹ or X² connect.

In some embodiments, X¹ is a covalent bond, —NH—, or —NMe-.

In some embodiments, X² is a covalent bond, —CH₂—, —CMe(OMe)-, —CMe(F)—, —CMe(CF₃)—, cyclopropylenyl, difluorocyclopropylenyl, —NH—, —NMe-, —N(COMe)-, —N(CF₃)—, —NEt-, —N(nPr)—, —N(nBu)-, —N(Ph)-, —N(3-pyridyl)-, —N(4-pyridyl)-, —N(SO₂Me)-, —N(CH₂CHF₂)—, —N(CH₂cyclopropyl)-, —N(CH₂Ph)-, —N(CH₂CONH₂)—, —N(CH₂SO₂Me)-, —N(CH₂CH₂CHF₂)—, —N(CH₂CH₂Ph)-, —N(CH₂CH₂CO₂H)—, —N(CH₂CH₂CONH₂)—, —N(CH₂CH₂CN)—, —N(CH₂CH₂OMe)-, —N(CH₂CH₂SO₂Me)-, —O—, —S—, or —S(O)₂—.

In some embodiments, X² represents spiro-fusion between the two rings that X² connects, e.g.,

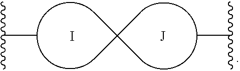

In some embodiment, X¹ and X² are as depicted in the compounds of Table 3, below.

As described above and defined herein, Y¹ is a C₁₋₃ hydrocarbon chain wherein each methylene is optionally substituted with —CR₂—, —CR(OR)—, —C(O)—, —C(NR)—, —C(NOR)—, —S(O)—, or —S(O)₂—.

In some embodiments, Y¹ is a C₁₋₃ hydrocarbon chain wherein each methylene is optionally substituted with —CR₂—, —CR(OR)—, —C(O)—, —C(NR)—, —C(NOR)—, —S(O)—, or —S(O)₂—.

In some embodiments, Y¹ is a C₁₋₃ hydrocarbon chain. In some embodiments, Y¹ is —CR₂—. In some embodiments, Y¹ is —CR(OR)—. In some embodiments, Y¹ is —C(O)—. In some embodiments, Y¹ is —C(NR)—. In some embodiments, Y¹ is —C(NOR)—. In some embodiments, Y¹ is —S(O)—. In some embodiments, Y¹ is —S(O)₂—.

In some embodiments, Y¹ is —CH₂—, —CH₂C(O)—, —NHCH₂C(O)—, —CH₂CH₂C(O)—, —CH₂CH(OH)C(O)—, —C(O)—, —C(NH)—, —C(NOH)—, —S(O)—, or —S(O)₂—.

In some embodiment, Y¹ is as depicted in the compounds of Table 3, below.

As described above and defined herein, s is 0 or 1.

In some embodiments, s is 0. In some embodiments, s is 1.

In some embodiment, s is as depicted in the compounds of Table 3, below.

As described above and defined herein, each of e, f, g, h, i, j, and k are independently 0, 1, 2, 3, or 4.

In some embodiments, e is 0. In some embodiments, e is 1. In some embodiments, e is 2. In some embodiments, e is 3. In some embodiments, e is 4.

In some embodiments, f is 0. In some embodiments, f is 1. In some embodiments, f is 2. In some embodiments, f is 3. In some embodiments, f is 4.

In some embodiments, g is 0. In some embodiments, g is 1. In some embodiments, g is 2. In some embodiments, g is 3. In some embodiments, g is 4.

In some embodiments, h is 0. In some embodiments, h is 1. In some embodiments, h is 2. In some embodiments, h is 3. In some embodiments, h is 4.

In some embodiments, i is 0. In some embodiments, i is 1. In some embodiments, i is 2. In some embodiments, i is 3. In some embodiments, i is 4.

In some embodiments, j is 0. In some embodiments, j is 1. In some embodiments, j is 2. In some embodiments, j is 3. In some embodiments, j is 4.

In some embodiments, k is 0. In some embodiments, k is 1. In some embodiments, k is 2. In some embodiments, k is 3. In some embodiments, k is 4.

In some embodiment, e, f, g, h, i, j, and k are as depicted in the compounds of Table 3, below.

In some embodiments, DBM is

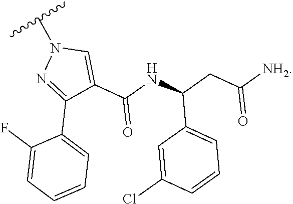 [SMILES: *n1cc(C(=O)N[C@@H](CC(N)=O)c2cccc(Cl)c2)c(-c2ccccc2F)n1]

In some embodiments, DBM is

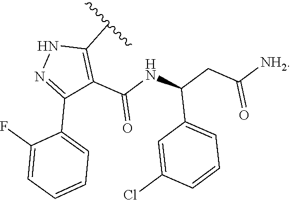 [SMILES: *c1[nH]nc(-c2ccccc2F)c1C(=O)N[C@@H](CC(N)=O)c1cccc(Cl)c1]

In some embodiments, DBM is

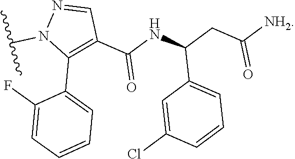 [SMILES: *n1ncc(C(=O)N[C@@H](CC(N)=O)c2cccc(Cl)c2)c1-c1ccccc1F]

In some embodiments, DBM is

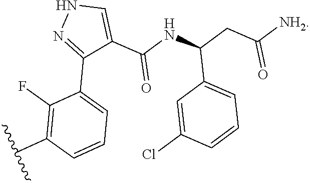 [SMILES: *c1cccc(-c2n[nH]cc2C(=O)N[C@@H](CC(N)=O)c2cccc(Cl)c2)c1F]

In some embodiments, DBM is

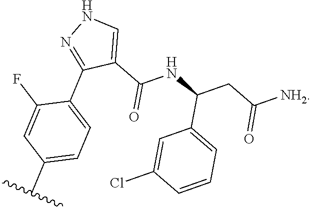 [SMILES: *c1ccc(-c2n[nH]cc2C(=O)N[C@@H](CC(N)=O)c2cccc(Cl)c2)c(F)c1]

In some embodiments, DBM is

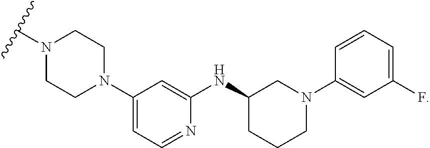 [SMILES: *N1CCN(c2ccnc(N[C@@H]3CCCN(c4cccc(F)c4)C3)c2)CC1]

In some embodiments, DBM is

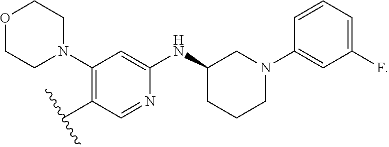 [SMILES: *c1cnc(N[C@@H]2CCCN(c3cccc(F)c3)C2)cc1N1CCOCC1]

In some embodiments, DBM is

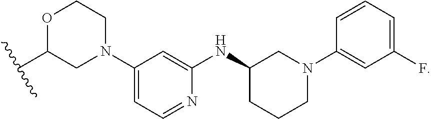 [SMILES: *C1CN(c2ccnc(N[C@@H]3CCCN(c4cccc(F)c4)C3)c2)CCO1]

In some embodiments, DBM is

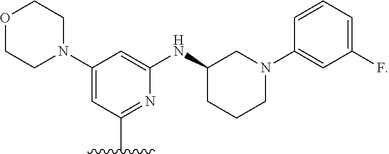 [SMILES: *c1cc(N2CCOCC2)cc(N[C@@H]2CCCN(c3cccc(F)c3)C2)n1]

In certain embodiments, the present invention provides a compound of formula I-s represented by any one of the following formulae:

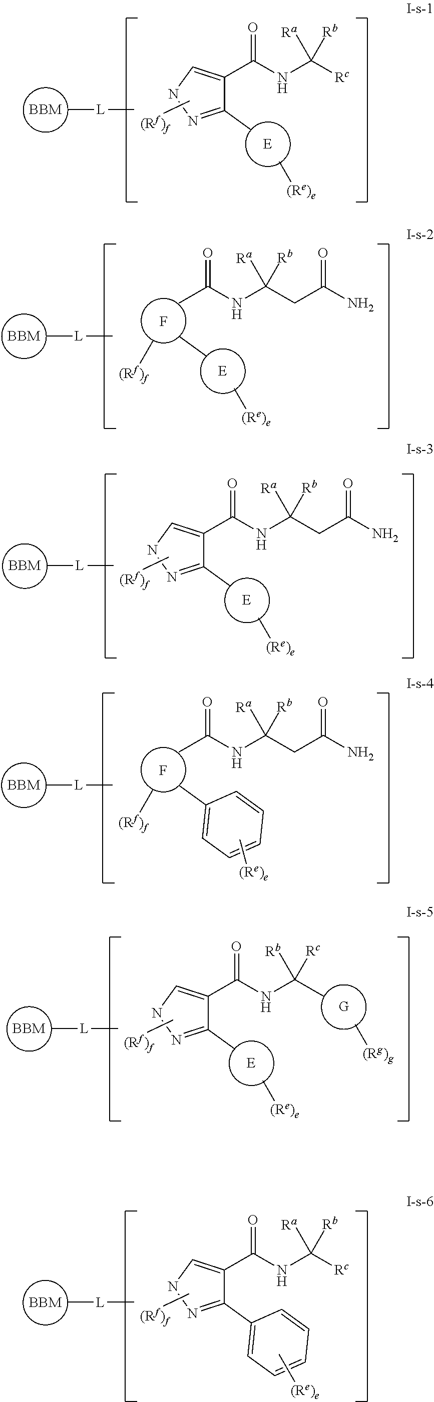

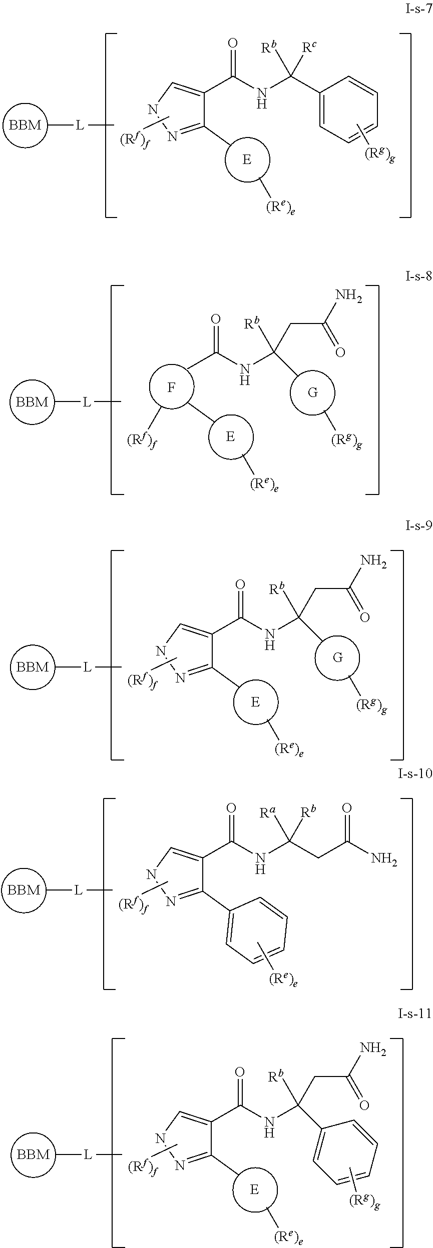

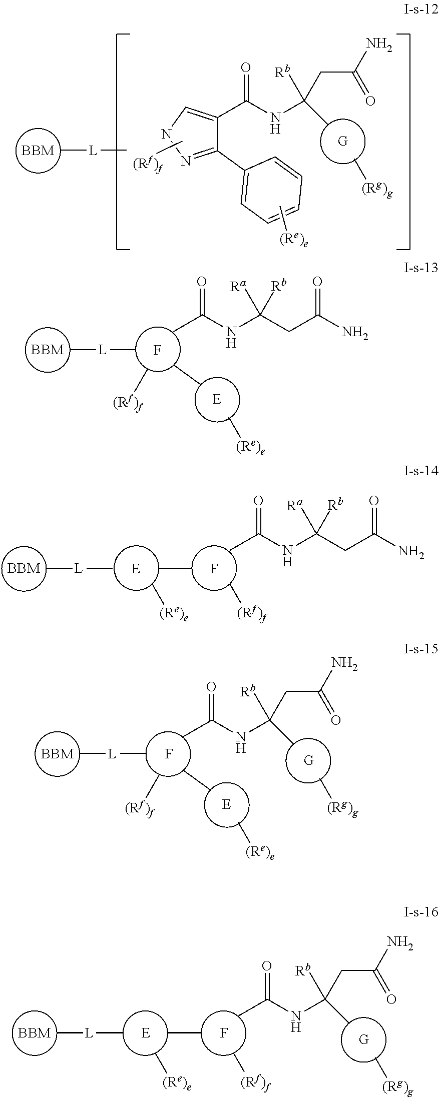

or a pharmaceutically acceptable salt thereof.

In certain embodiments, the present invention provides a compound of formula I-t represented by any one of the following formulae:

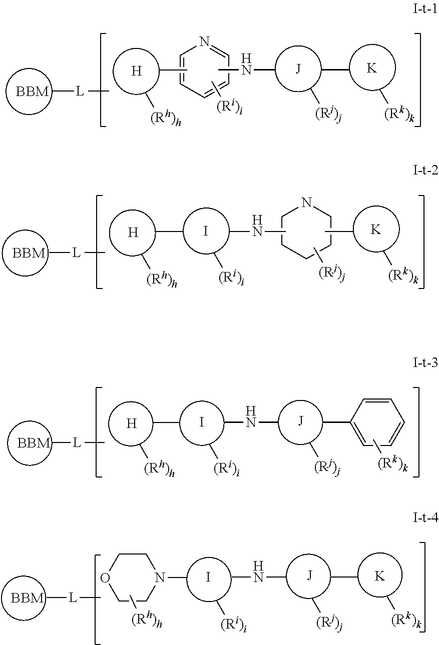

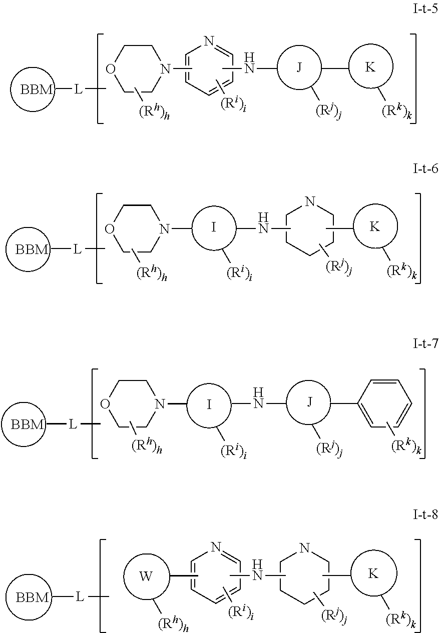

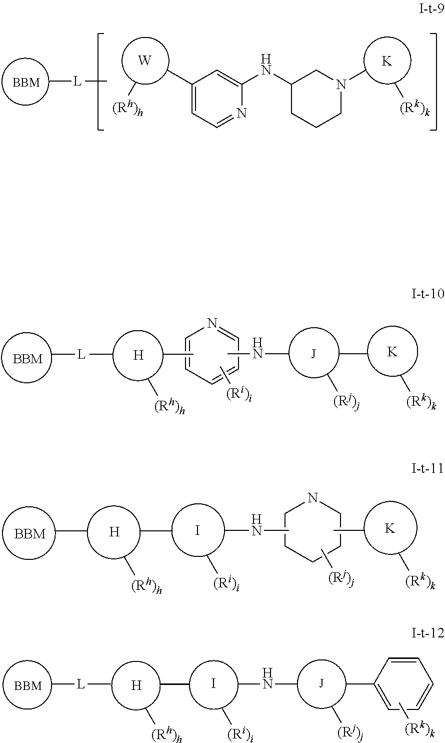

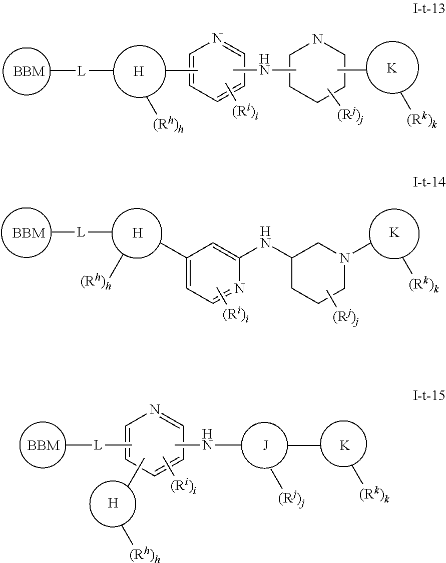

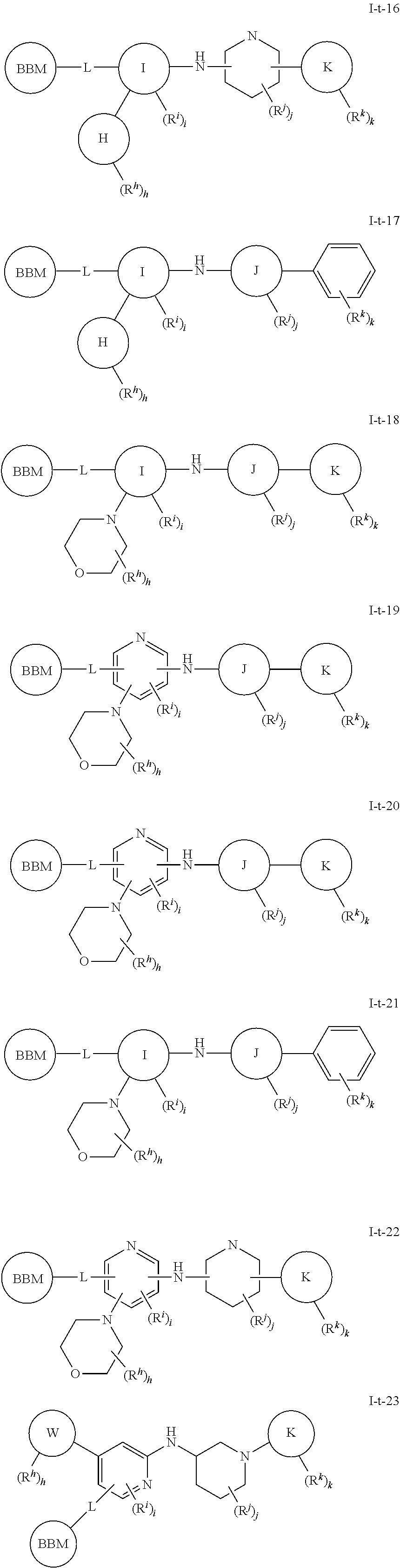

or a pharmaceutically acceptable salt thereof.

As defined above and described herein, DBM is further optionally substituted with

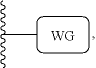

wherein

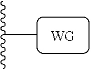

is a warhead group, attached to a modifiable carbon, oxygen, nitrogen or sulfur atom in formula I-s or I-t or substitution or replacement of any defined group in formula I-s or I-t (e.g., substitution or replacement of R^{e}, R^{f}, R^{g}, R^{h}, Rⁱ, R^{j}, or R^{k}).

In some embodiments, the warhead group is -L²-Y, wherein:
L² is a covalent bond or a bivalent C₁₋₈ saturated or unsaturated, straight or branched, hydrocarbon chain, wherein one, two, or three methylene units of L² are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—;
Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 R^{e} groups; and
each R^{e} is independently selected from -Q-Z, oxo, NO₂, halogen, CN, a suitable leaving group, or a C₁₋₆ aliphatic optionally substituted with oxo, halogen, NO₂, or CN, wherein:
   Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O, —SO—, or —SO, —N(R)C(O)—, —C(O)N(R)—, —N(R)SO₂—, or —SO₂N(R)—; and
Z is hydrogen or C₁₋₆ aliphatic optionally substituted with oxo, halogen, NO₂, or CN.

In certain embodiments, L² is a covalent bond.

In certain embodiments, L² is a bivalent C₁₋₈ saturated or unsaturated, straight or branched, hydrocarbon chain. In certain embodiments, L² is —CH₂—.

In certain embodiments, L² is a covalent bond, —CH₂—, —NH—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—.

In some embodiments, L² is a bivalent C₂₋₈ straight or branched, hydrocarbon chain wherein L² has at least one double bond and one or two additional methylene units of L² are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—.

In certain embodiments, L² is a bivalent C₂₋₈ straight or branched, hydrocarbon chain wherein L² has at least one double bond and at least one methylene unit of L² is replaced by —C(O)—, —NRC(O), —C(O)NR—, —N(R)SO₂—, —SO₂N(R)—, —S—, —S(O)—, —SO₂—, —OC(O)—, or —C(O)O—, and one or two additional methylene units of L² are optionally and independently replaced by cyclopropylene, —O—, —N(R)—, or —C(O)—.

In some embodiments, L² is a bivalent C₂₋₈ straight or branched, hydrocarbon chain wherein L² has at least one double bond and at least one methylene unit of L² is replaced by —C(O)—, and one or two additional methylene units of L² are optionally and independently replaced by cyclopropylene, —O—, —N(R)—, or —C(O)—.

As described above, in certain embodiments, L² is a bivalent C₂₋₈ straight or branched, hydrocarbon chain wherein L² has at least one double bond. One of ordinary skill in the art will recognize that such a double bond may exist within the hydrocarbon chain backbone or may be “exo” to the backbone chain and thus forming an alkylidene group. By way of example, such an L² group having an alkylidene branched chain includes —CH₂C(═CH₂)CH₂—. Thus, in some embodiments, L² is a bivalent C₂₋₈ straight or branched, hydrocarbon chain wherein L¹ has at least one alkylidenyl double bond. Exemplary L² groups include —NHC(O)C(═CH₂)CH₂—.

In certain embodiments, L² is a bivalent C₂₋₈ straight or branched, hydrocarbon chain wherein L² has at least one double bond and at least one methylene unit of L² is replaced by —C(O)—. In certain embodiments, L² is —C(O)CH═CH(CH₃)—, —C(O)CH═CHCH₂NH(CH₃)—, —C(O)CH═CH(CH₃)—, —C(O)CH═CH—, —CH₂C(O)CH═CH—, —CH₂C(O)CH═CH(CH₃)—, —CH₂CH═C(O)CH═CH—, —CH₂CH₂C(O)CH═CHCH₂—, —CH₂CH₂C(O)CH—CHCH₂NH(CH₃)—, or CH₂CH₂C(O)CH═CH(CH₃)—, or —CH(CH₃)OC(O)CH═CH—.

In certain embodiments, L² is a bivalent C₂₋₈ straight or branched, hydrocarbon chain wherein L² has at least one double bond and at least one methylene unit of L¹ is replaced by —OC(O)—.

In some embodiments, L² is a bivalent C₂₋₈ straight or branched, hydrocarbon chain wherein L² has at least one double bond and at least one methylene unit of L¹ is replaced by —NRC(O)—, —C(O)NR—, —N(R)SO₂—, —SO₂N(R)—, —S—, —S(O)—, —SO₂—, —OC(O)—, or —C(O)O—, and one or two additional methylene units of L¹ are optionally and independently replaced by cyclopropylene, —O—, —N(R)—, or —C(O)—. In some embodiments, L² is —CH₂OC(O)CH═CHCH₂—, —CH₂—OC(O)CH═CH, or CH(CH═CH₂)OC(O)CH═CH—.

In certain embodiments, L² is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-, wherein each R is independently hydrogen or optionally substituted C₁₋₆ aliphatic.

In certain embodiments, L² is —NHC(O)CH—CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)C(O)—, —NHC(O)CH═CHCH₂N(CH₃)—, —NHSO₂CH═CH—, —NHSO₂CH═CHCH₂—, —NHC(O)CH—CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-.

In some embodiments, L² is a bivalent C₂₋₈ straight or branched, hydrocarbon chain wherein L² has at least one triple bond. In certain embodiments, L² is a bivalent C₂₋₈ straight or branched, hydrocarbon chain wherein L² has at least one triple bond and one or two additional methylene units of L² are optionally and independently replaced by —NRC(O)—, —C(O)NR—, —S—, —S(O)—, —SO₂—, —C(═S)—, —C(═NR)—, —O—, —N(R)—, or —C(O)—. In some embodiments, L² has at least one triple bond and at least one methylene unit of L² is replaced by —N(R)—, —N(R)C(O)—, —C(O)—, —C(O)O—, or —OC(O)—, or —O—.

Exemplary L² groups include —C≡C—, —C≡CCH₂N(isopropyl)-, —NHC(O)C≡CCH₂CH₂—, —CH₂—C≡C≡CH₂—, —C═CCH₂O—, —CH₂C(O)C═C—, —C(O)C═C—, or —CH₂OC(═O)C═C—.

In certain embodiments, L² is a bivalent C₂₋₈ straight or branched, hydrocarbon chain wherein one methylene unit of L² is replaced by cyclopropylene and one or two additional methylene units of L² are independently replaced by —C(O)—, —NRC(O)—, —C(O)NR—, —N(R)SO₂—, or —SO₂N(R)—. Exemplary L² groups include —NHC(O)-cyclopropylene-SO₂— and —NHC(O)-cyclopropylene-.

As defined generally above, Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with at 1-4 R^{e} groups, each R^{e} is independently selected from -Q-Z, oxo, NO₂, halogen, CN, a suitable leaving group, or C₁₋₆ aliphatic, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂, —N(R)C(O)—, —C(O)N(R)—, —N(R)SO₂—, or —SO₂N(R)—; and, Z is hydrogen or C₁₋₆ aliphatic optionally substituted with oxo, halogen, NO₂, or CN.

In certain embodiments, Y is hydrogen.

In certain embodiments, Y is C₁₋₆ aliphatic optionally substituted with oxo, halogen, NO₂, or CN. In some embodiments, Y is C₂₋₆alkenyl optionally substituted with oxo, halogen, NO₂, or CN. In other embodiments, Y is C₂₋₆alkynyl optionally substituted with oxo, halogen, NO₂, or CN. In some embodiments, Y is C₂₋₆alkenyl. In other embodiments, Y is C₂₋₄ alkynyl.

In other embodiments, Y is C₁₋₆ alkyl substituted with oxo, halogen, NO₂, or CN. Such Y groups include —CH₂F, —CH₂Cl, —CH₂CN, and —CH₂NO₂.

In certain embodiments, Y is a saturated 3-6 membered monocyclic ring having 0-3 heteroatoms independently selected from nitrogen, oxygen, or sulfur, wherein Y is substituted with 1-4 R^{e} groups, wherein each R^{e} is as defined above and described herein.

In some embodiments, Y is a saturated 3-4 membered heterocyclic ring having 1 heteroatom selected from oxygen or nitrogen wherein said ring is substituted with 1-2 R^{e} groups, wherein each R^{e} is as defined above and described herein. Exemplary such rings are epoxide and oxetane rings, wherein each ring is substituted with 1-2 R^{e} groups, wherein each R^{e} is as defined above and described herein.

In other embodiments, Y is a saturated 5-6 membered heterocyclic ring having 1-2 heteroatom selected from oxygen or nitrogen wherein said ring is substituted with 1-4 R^{e} groups, wherein each R^{e} is as defined above and described herein. Such rings include piperidine and pyrrolidine, wherein each ring is substituted with 1-4 R^{e} groups, wherein each R^{e} is as defined above and described herein. In certain embodiments, Y is

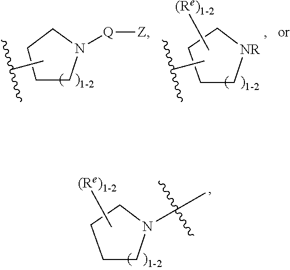

wherein each R, Q, Z, and R^{e} is as defined above and described herein.

In some embodiments, Y is a saturated 3-6 membered carbocyclic ring, wherein said ring is substituted with 1-4 R^{e} groups, wherein each R^{e} is as defined above and described herein. In certain embodiments, Y is cyclopropyl, cyclobutyl, cyclopentyl, or cyclohexyl, wherein each ring is substituted with 1-4 R^{e} groups, wherein each R^{e} is as defined above and described herein. In certain embodiments, Y is

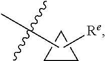

wherein R^{e} is as defined above and described herein.

In certain embodiments, Y is cyclopropyl optionally substituted with halogen, CN or NO₂.

In certain embodiments, Y is a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein.

In some embodiments, Y is a partially unsaturated 3-6 membered carbocyclic ring, wherein said ring is substituted with 1-4 R^{e} groups, wherein each R^{e} is as defined above and described herein. In some embodiments, Y is cyclopropenyl, cyclobutenyl, cyclopentenyl, or cyclohexenyl wherein each ring is substituted with 1-4 R^{e} groups, wherein each R^{e} is as defined 0-3 above and described herein. In certain embodiments, Y is

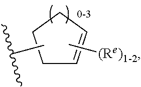

wherein each R^{e} is as defined above and described herein.

In certain embodiments, Y is a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein. In certain embodiments, Y is selected from:

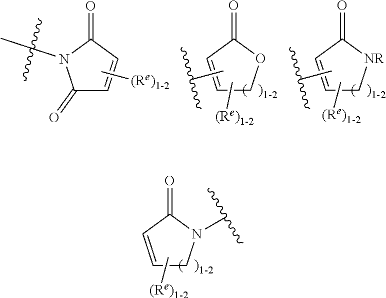

wherein each R and R^{e} is as defined above and described herein.

In certain embodiments, Y is a 6-membered aromatic ring having 0-2 nitrogens wherein said ring is substituted with 1-4 R^{e} groups, wherein each R^{e} group is as defined above and described herein. In certain embodiments, Y is phenyl, pyridyl, or pyrimidinyl, wherein each ring is substituted with 1-4 R^{e} groups, wherein each R^{e} is as defined above and described herein.

In some embodiments, Y is selected from:

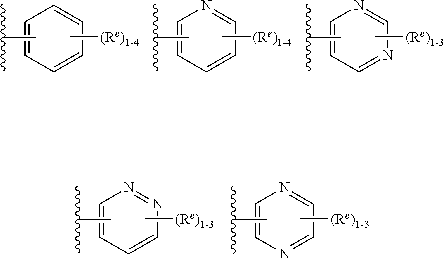

wherein each R^{e} is as defined above and described herein.

In other embodiments, Y is a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein. In some embodiments, Y is a 5 membered partially unsaturated or aryl ring having 1-3 heteroatoms independently selected from nitrogen, oxygen, and sulfur, wherein said ring is substituted with 1-4 R^{e} groups, wherein each R^{e} group is as defined above and described herein. Exemplary such rings are isoxazolyl, oxazolyl, thiazolyl, imidazolyl, pyrazolyl, pyrrolyl, furanyl, thienyl, triazole, thiadiazole, and oxadiazole, wherein each ring is substituted with 1-3 R^{e} groups, wherein each R^{e} group is as defined above and described herein. In certain embodiments, Y is selected from:

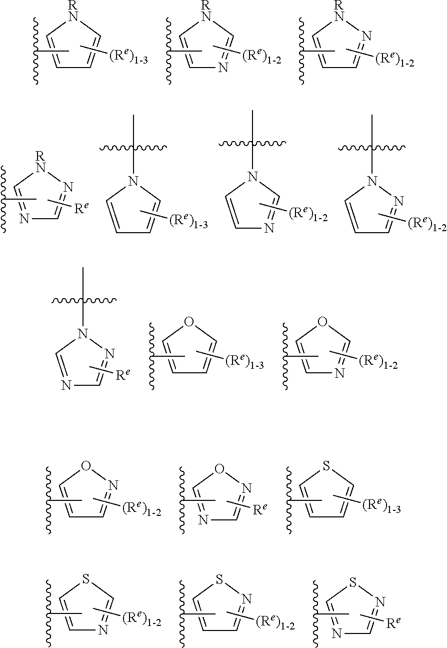

wherein each R and R^{e} is as defined above and described herein.

In certain embodiments, Y is an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein. According to another aspect, Y is a 9-10 membered bicyclic, partially unsaturated, or aryl ring having 1-3 heteroatoms independently selected from nitrogen, oxygen, or sulfur, wherein said ring is substituted with 1-4 R^{e} groups, wherein R^{e} is as defined above and described herein. Exemplary such bicyclic rings include 2,3-dihydrobenzo[d]isothiazole, wherein said ring is substituted with 1-4 R^{e} groups, wherein R^{e} is as defined above and described herein.

As defined generally above, each R^{e} group is independently selected from -Q-Z, oxo, NO₂, halogen, CN, a suitable leaving group, or C₁₋₆ aliphatic optionally substituted with oxo, halogen, NO₂, or CN, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and Z is hydrogen or C₁₋₆ aliphatic optionally substituted with oxo, halogen, NO₂, or CN.

In certain embodiments, R^{e} is C₁₋₆ aliphatic optionally substituted with oxo, halogen, NO₂, or CN. In other embodiments, R^{e} is oxo, NO₂, halogen, or CN.

In some embodiments, R^{e} is -Q-Z, wherein Q is a covalent bond and Z is hydrogen (i.e., R^{e} is hydrogen). In other embodiments, R^{e} is -Q-Z, wherein Q is a bivalent C₁₋₆ saturated or unsaturated, straight or branched, hydrocarbon chain, wherein one or two methylene units of Q are optionally and independently replaced by —NR—, —NRC(O)—, —C(O)NR—, —S—, —O—, —C(O)—, —SO—, or —SO₂—. In other embodiments, Q is a bivalent C₂₋₆ straight or branched, hydrocarbon chain having at least one double bond, wherein one or two methylene units of Q are optionally and independently replaced by —NR—, —NRC(O)—, —C(O)NR—, —S—, —O—, —C(O)—, —SO—, or —SO₂—. In certain embodiments, the Z moiety of the R^{e} group is hydrogen. In some embodiments, -Q-Z is —NHC(O)CH═CH₂ or —C(O)CH═CH₂.

In certain embodiments, each R^{e} is independently selected from oxo, NO₂, CN, fluoro, chloro, —NHC(O)CH═CH₂, —C(O)CH═CH₂, —CH₂CH═CH₂, —C═CH, —C(O)OCH₂Cl, —C(O)OCH₂F, —C(O)OCH₂CN, —C(O)CH₂Cl, —C(O)CH₂F, —C(O)CH₂CN, or —CH₂C(O)CH₃.

In certain embodiments, R^{e} is a suitable leaving group, i.e., a group that is subject to nucleophilic displacement. A “suitable leaving” is a chemical group that is readily displaced by a desired incoming chemical moiety such as the thiol moiety of a cysteine of interest. Suitable leaving groups are well known in the art, e.g., see, “Advanced Organic Chemistry,” Jerry March 5^{th} Ed., pp. 351-357, John Wiley and Sons, N.Y. Such leaving groups include, but are not limited to, halogen, alkoxy, sulphonyloxy, optionally substituted alkylsulphonyloxy, optionally substituted alkenylsulfonyloxy, optionally substituted arylsulfonyloxy, acyl, and diazonium moieties. Examples of suitable leaving groups include chloro, iodo, bromo, fluoro, acetoxy, methanesulfonyloxy (mesyloxy), tosyloxy, triflylovy, nitro-phenylsulfonyloxy (nosyloxy), and bromo-phenylsulfonyloxy (brosyloxy).

In certain embodiments, the following embodiments and combinations of -L²-Y apply:
(a) L¹ is a bivalent C₂₋₈ straight or branched, hydrocarbon chain wherein L² has at least one double bond and one or two additional methylene units of L¹ are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—; and Y is hydrogen or C₁₋₆ aliphatic optionally substituted with oxo, halogen, NO₂, or CN; or
(b) L² is a bivalent C₂₋₈ straight or branched, hydrocarbon chain wherein L² has at least one double bond and at least one methylene unit of L² is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L² are optionally and independently replaced by cyclopropylene, —O—, —N(R)—, or —C(O)—; and Y is hydrogen or C₁₋₆ aliphatic optionally substituted with oxo, halogen, NO₂, or CN; or
(c) L¹ is a bivalent C₂₋₈ straight or branched, hydrocarbon chain wherein L² has at least one double bond and at least one methylene unit of L² is replaced by —C(O)—, and one or two additional methylene units of L¹ are optionally and independently replaced by cyclopropylene, —O—, —N(R)—, or —C(O)—; and Y is hydrogen or C₁₋₆ aliphatic optionally substituted with oxo, halogen, NO₂, or CN; or
(d) L¹ is a bivalent C₂₋₈ straight or branched, hydrocarbon chain wherein L² has at least one double bond and at least one methylene unit of L² is replaced by —C(O)—; and Y is hydrogen or C₁₋₆ aliphatic optionally substituted with oxo, halogen, NO₂, or CN; or
(e) L¹ is a bivalent C₂₋₈ straight or branched, hydrocarbon chain wherein L² has at least one double bond and at least one methylene unit of L² is replaced by —OC(O)—; and Y is hydrogen or C₁₋₆ aliphatic optionally substituted with oxo, halogen, NO₂, or CN; or
(f) L² is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)—, —NRC(O)(C═N₂) C(O)—, —NRC(O)CH═CHCH₂N(CH₃)—, —NRSO₂CH═CH—, NRSO₂CH═CHCH₂, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-; wherein R is H or optionally substituted C₁₋₆ aliphatic; and Y is hydrogen or C₁₋₆ aliphatic optionally substituted with oxo, halogen, NO₂, or CN; or
(g) L² is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSOCH═CHCH₂—, —NHC(O)(C═N₂)—, —NHC(O)(C═N₂)C(O)—, —NHC(O)CH═CHCH₂N(CH₃)—, —NHSO₂CH═CH—, NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-; and Y is hydrogen or C₁₋₆ aliphatic optionally substituted with oxo, halogen, NO₂, or CN; or
(h) L² is a bivalent C₂₋₈ straight or branched, hydrocarbon chain wherein L² has at least one alkylidenyl double bond and at least one methylene unit of L² is replaced by —C(O)—, —NRC(O)—, —C(O)NR—, —N(R)SO₂, —SO₂N(R)—, —S—, —S(O)—, —SO₂—, —OC(O)—, or —C(O)O—, and one or two additional methylene units of L² are optionally and independently replaced by cyclopropylenc, —O—, —N(R)—, or —C(O)—; and Y is hydrogen or C₁₋₆ aliphatic optionally substituted with oxo, halogen, NO₂, or CN; or
(i) L² is a bivalent C₂₋₈ straight or branched, hydrocarbon chain wherein L² has at least one triple bond and one or two additional methylene units of L² are optionally and independently replaced by —NRC(O)—, —C(O)NR—, —N(R)SO₂—, —SO₂N(R)—, —S—, —S(O)—, —SO₂—, —OC(O)—, or —C(O)O—, and Y is hydrogen or C₁₋₆ aliphatic optionally substituted with oxo, halogen, NO₂, or CN; or
(j) L² is —C≡C—, —C≡CCH₂N(isopropyl)-, —NHC(O)C≡CCH₂CH₂—, —CH₂—C≡C≡CH₂—, —C≡CCH₂O—, —CH₂C(O)C≡C—, —C(O)C≡C—, or —CH₂C(═O)C≡C—; and Y is hydrogen or C₁₋₆ aliphatic optionally substituted with oxo, halogen, NO₂, or CN; or
(k) L² is a bivalent C₂₋₈ straight or branched, hydrocarbon chain wherein one methylene unit of L² is replaced by cyclopropylene and one or two additional methylene units of L² are independently replaced by —NRC(O)—, —C(O)NR—, —N(R)SO₂—, —SO₂N(R)—, —S—, —S(O)—, —SO₂—, —OC(O)—, or —C(O)O—; and Y is hydrogen or C₁₋₆ aliphatic optionally substituted with oxo, halogen, NO₂, or CN; or
(l) L² is a covalent bond and Y is selected from:
   (i) C₁₋₆ alkyl substituted with oxo, halogen, NO₂, or CN;
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or
   (vi)

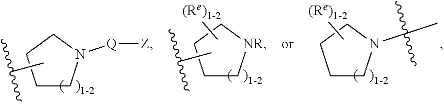

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or
(x)

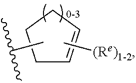

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or
   (xii)

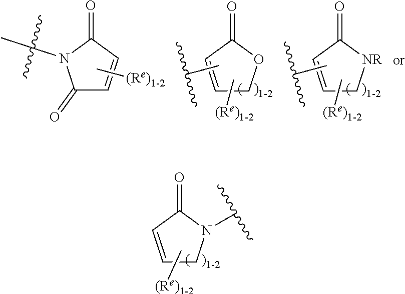

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or
(xiv)

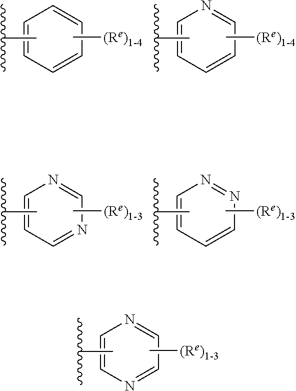

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or
(xvi)

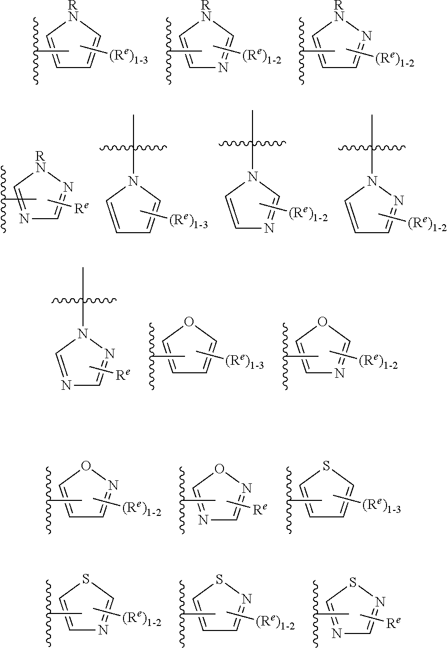

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(m) L² is —C(O)— and Y is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or
   (vi)

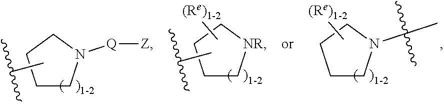

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or
(x)

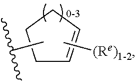

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or
(xii)

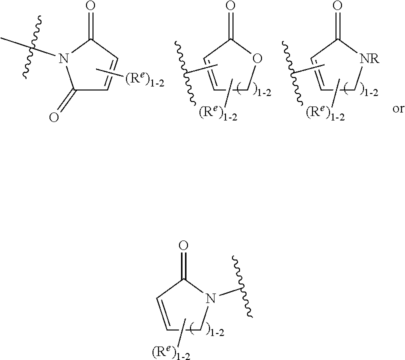

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or
(xiv)

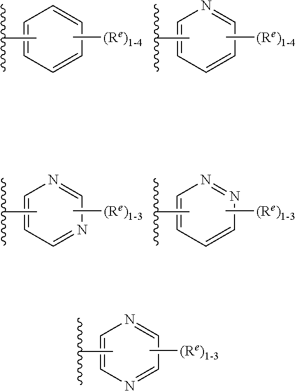

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or
(xvi)

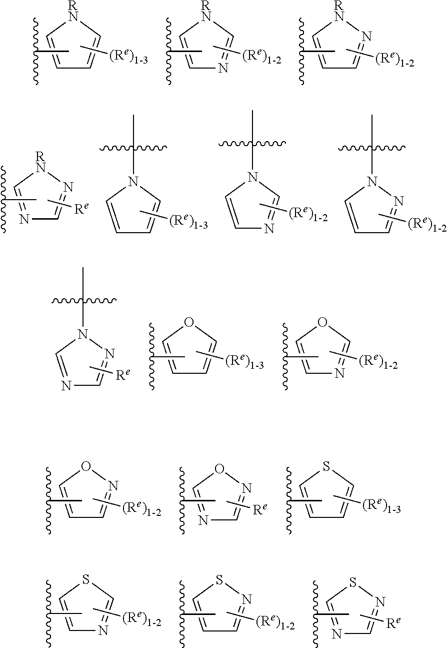

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(n) L² is —N(R)C(O)— and Y is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or
   (vi)

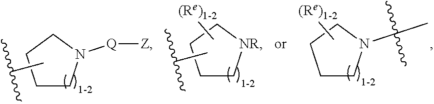

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or
(x)

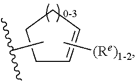

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or
(xii)

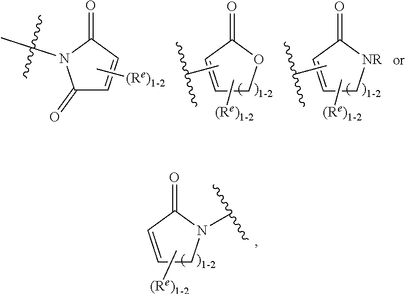

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or
(xiv)

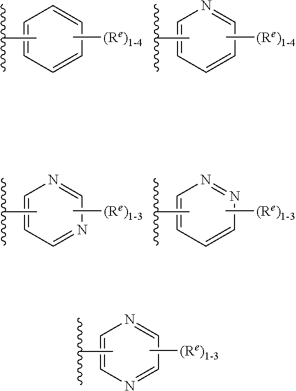

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or
(xvi)

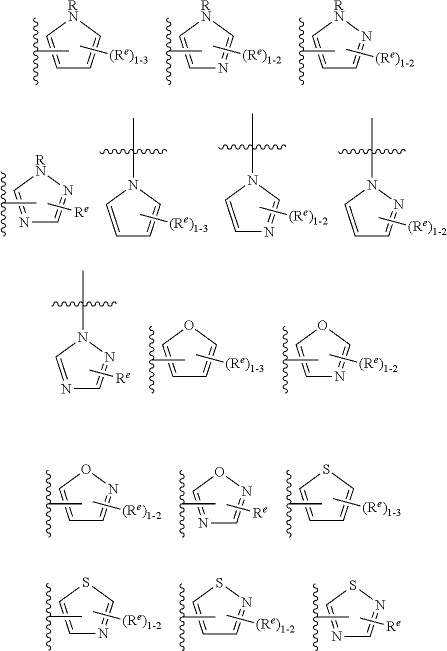

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(o) L² is a bivalent C₁₋₈ saturated or unsaturated, straight or branched, hydrocarbon chain; and Y is selected from:
   (i) C₁₋₆ alkyl substituted with oxo, halogen, NO₂, or CN;
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or
   (vi)

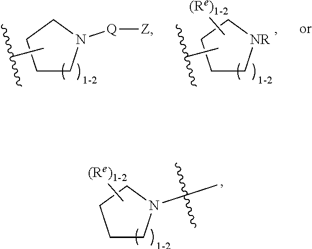

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or
(x)

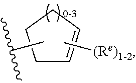

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 Rₑ groups, wherein each Rₑ is as defined above and described herein; or
(xii)

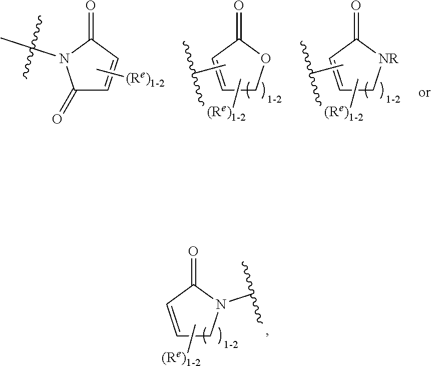

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or
(xiv)

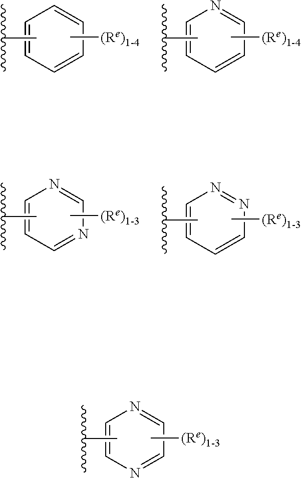

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or
(xvi)

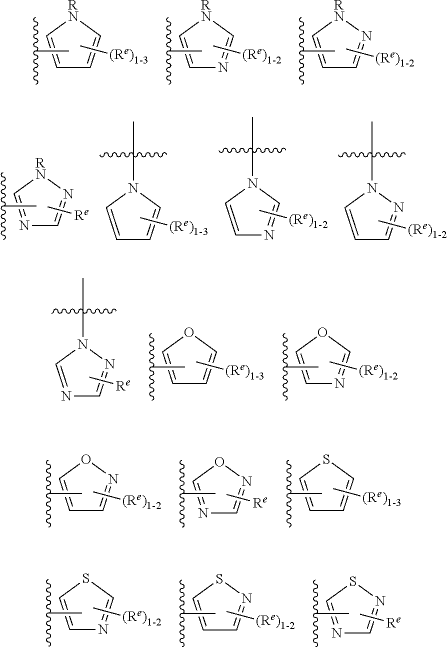

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(p) L² is a covalent bond, —CH₂—, —NH—, —C(O)—, —CH₂NH—, —NHCH₂—, —NHC(O)—, —NHC(O)CH₂OC(O)—, —CH₂NHC(O)—, —NHSO₂—, —NHSO₂CH₂, —NHC(O)CH₂OC(O)—, or —SO₂NH—; and Y is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or
   (vi)

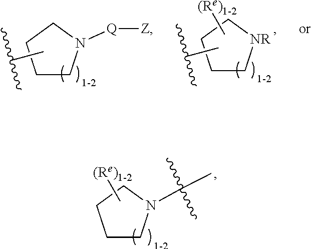

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or
(x)

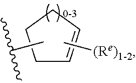

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or
   (xii)

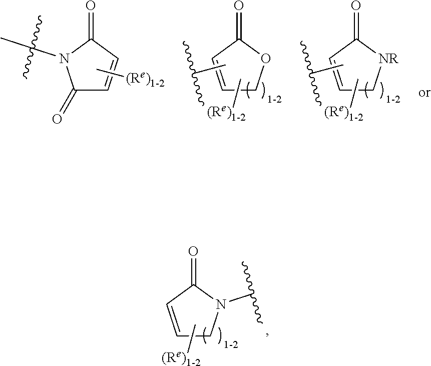

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or
(xiv)

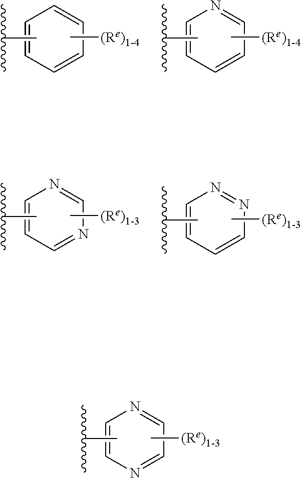

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or
(xvi)

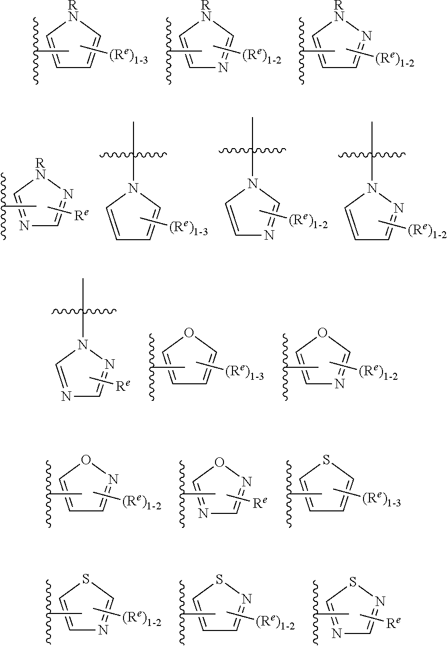

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein.

In certain embodiments, the Y group is selected from those set forth in Table 3A below, wherein each wavy line indicates the point of attachment to the rest of the molecule.

TABLE 3A
Exemplary Y groups

| | |
|---|---|
| 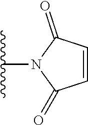 [SMILES: *N1C(=O)C=CC1=O] | a |
| 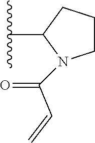 [SMILES: *C1CCCN1C(=O)C=C] | b |
| 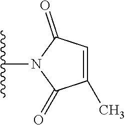 [SMILES: *N1C(=O)C=C(C)C1=O] | c |
| 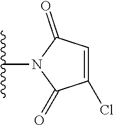 [SMILES: *N1C(=O)C=C(Cl)C1=O] | d |
| 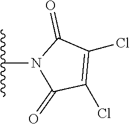 [SMILES: *N1C(=O)C(Cl)=C(Cl)C1=O] | e |
| 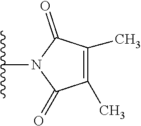 [SMILES: *N1C(=O)C(C)=C(C)C1=O] | f |
| 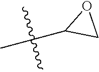 [SMILES: *C1CO1] | g |
| 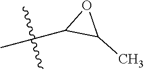 [SMILES: *C1OC1C] | h |
| 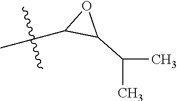 [SMILES: *C1OC1C(C)C] | i |
| 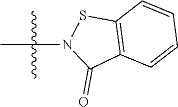 [SMILES: *n1sc2ccccc2c1=O] | j |
| 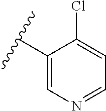 [SMILES: *c1cnccc1Cl] | k |
| 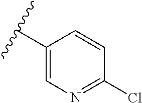 [SMILES: *c1ccc(Cl)nc1] | l |
| 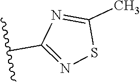 [SMILES: *c1nsc(C)n1] | m |
| 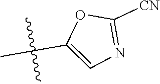 [SMILES: *c1cnc(C#N)o1] | n |
| 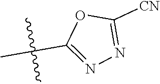 [SMILES: *c1nnc(C#N)o1] | o |
| 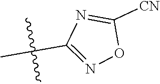 [SMILES: *c1noc(C#N)n1] | p |
| 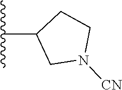 [SMILES: *C1CCN(C#N)C1] | q |
| 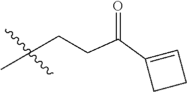 [SMILES: *CCC(=O)C1=CCC1] | r |
| 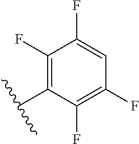 [SMILES: *c1c(F)c(F)cc(F)c1F] | s |
| 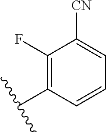 [SMILES: *c1cccc(C#N)c1F] | t |
| 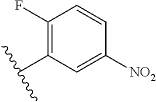 [SMILES: *c1cc([N+](=O)[O-])ccc1F] | u |
| 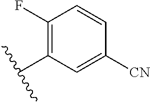 [SMILES: *c1cc(C#N)ccc1F] | v |
| 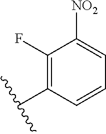 [SMILES: *c1cccc([N+](=O)[O-])c1F] | w |
| 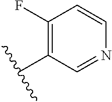 [SMILES: *c1cnccc1F] | x |
| 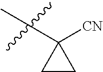 [SMILES: *C1(C#N)CC1] | y |
| 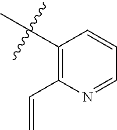 [SMILES: *c1cccnc1C=C] | z |
| 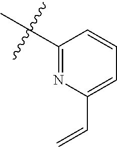 [SMILES: *c1cccc(C=C)n1] | aa |
| 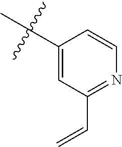 [SMILES: *c1ccnc(C=C)c1] | bb |
| 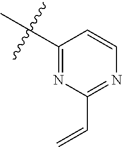 [SMILES: *c1ccnc(C=C)n1] | cc |
| 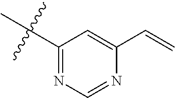 [SMILES: *c1cc(C=C)ncn1] | dd |
| 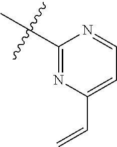 [SMILES: *c1nccc(C=C)n1] | ee |
| 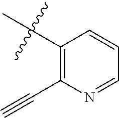 [SMILES: *c1cccnc1C#C] | ff |
| 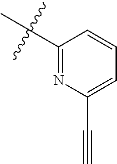 [SMILES: *c1cccc(C#C)n1] | gg |
| 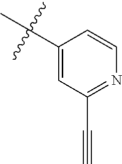 [SMILES: *c1ccnc(C#C)c1] | hh |
| 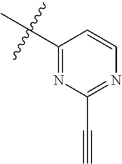 [SMILES: *c1ccnc(C#C)n1] | ii |
| 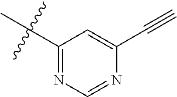 [SMILES: *c1cc(C#C)ncn1] | jj |
| 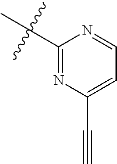 [SMILES: *c1nccc(C#C)n1] | kk |
| 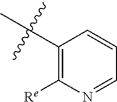 | ll |
| 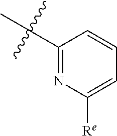 | mm |
| 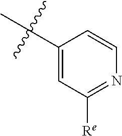 | nn |
| 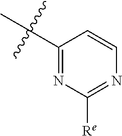 | oo |
| 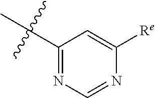 | pp |
| 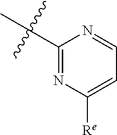 | qq |
| 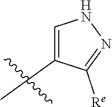 | rr |
| 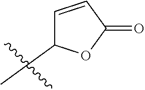 [SMILES: *C1C=CC(=O)O1] | ss |
| 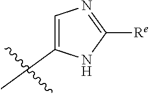 | tt |
| 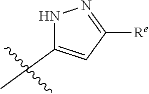 | uu |
| 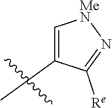 | vv |
| 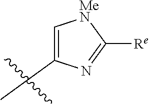 | ww |
| 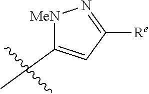 | xx |
| 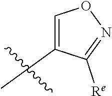 | yy |
| 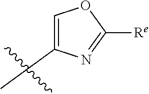 | zz |
| 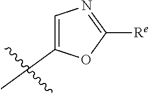 | aaa |
| 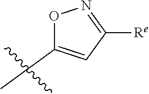 | bbb |
| 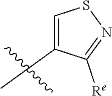 | ccc |
| 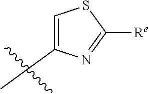 | ddd |
| 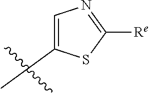 | eee |
| 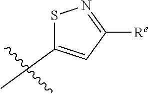 | fff |
| 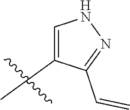 [SMILES: *c1c[nH]nc1C=C] | ggg |
| 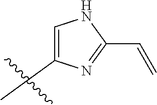 [SMILES: *c1c[nH]c(C=C)n1] | hhh |
| 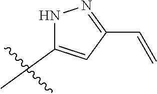 [SMILES: *c1cc(C=C)n[nH]1] | iii |
| 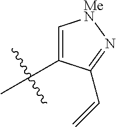 [SMILES: *c1cn(C)nc1C=C] | jjj |
| 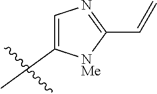 [SMILES: *c1cnc(C=C)n1C] | kkk |
| 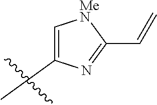 [SMILES: *c1cn(C)c(C=C)n1] | lll |
| 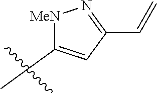 [SMILES: *c1cc(C=C)nn1C] | mmm |
| 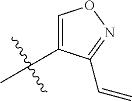 [SMILES: *c1conc1C=C] | nnn |
| 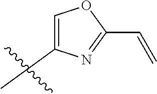 [SMILES: *c1coc(C=C)n1] | ooo |
| 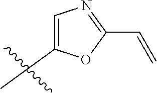 [SMILES: *c1cnc(C=C)o1] | ppp |
| 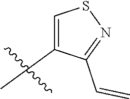 [SMILES: *c1csnc1C=C] | qqq |
| 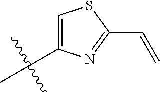 [SMILES: *c1csc(C=C)n1] | rrr |
| 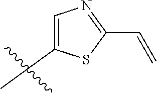 [SMILES: *c1cnc(C=C)s1] | sss |
| 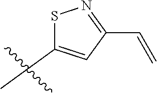 [SMILES: *c1cc(C=C)ns1] | ttt |
| 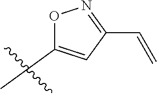 [SMILES: *c1cc(C=C)no1] | uuu |
| 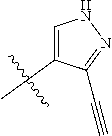 [SMILES: *c1c[nH]nc1C#C] | vvv |
| 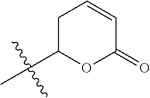 [SMILES: *C1CC=CC(=O)O1] | qqq |
| 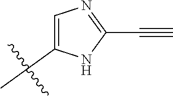 [SMILES: *c1cnc(C#C)[nH]1] | www |
| 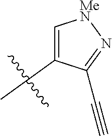 [SMILES: *c1cn(C)nc1C#C] | xxx |
| 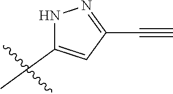 [SMILES: *c1cc(C#C)n[nH]1] | yyy |
| 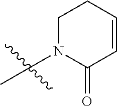 [SMILES: *N1CCC=CC1=O] | zzz |
| 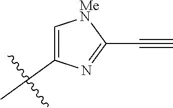 [SMILES: *c1cn(C)c(C#C)n1] | aaaa |
| 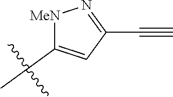 [SMILES: *c1cc(C#C)nn1C] | bbbb |
| 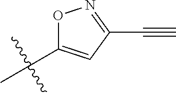 [SMILES: *c1cc(C#C)no1] | cccc |
| 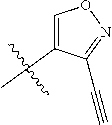 [SMILES: *c1conc1C#C] | dddd |
| 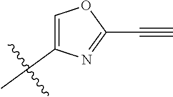 [SMILES: *c1coc(C#C)n1] | eeee |
| 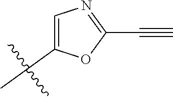 [SMILES: *c1cnc(C#C)o1] | ffff |
| 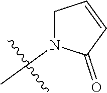 [SMILES: *N1CC=CC1=O] | gggg |
| 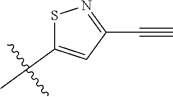 [SMILES: *c1cc(C#C)ns1] | hhhh |
| 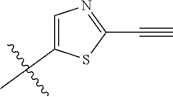 [SMILES: *c1cnc(C#C)s1] | iiii |
| 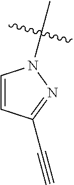 [SMILES: *n1ccc(C#C)n1] | jjjj |
| 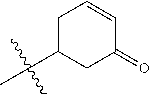 [SMILES: *C1CC=CC(=O)C1] | kkkk |
| 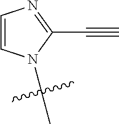 [SMILES: *n1ccnc1C#C] | llll |
| 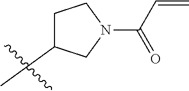 [SMILES: *C1CCN(C(=O)C=C)C1] | nnnn |
| 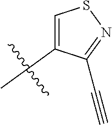 [SMILES: *c1csnc1C#C] | nnnn |
| 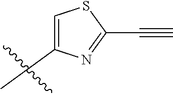 [SMILES: *c1csc(C#C)n1] | oooo |
| 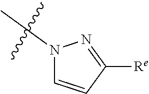 | pppp |
| 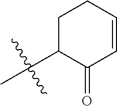 [SMILES: *C1CCC=CC1=O] | qqqq |
| 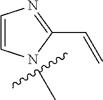 | rrrr |
| 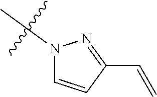 [SMILES: *n1ccc(C=C)n1] | ssss |
| 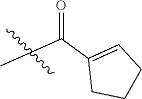 [SMILES: *C(=O)C1=CCCC1] | tttt |
| 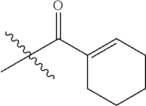 [SMILES: *C(=O)C1=CCCCC1] | uuuu |
| 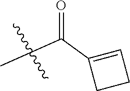 [SMILES: *C(=O)C1=CCC1] | vvvv |
| 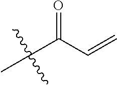 [SMILES: *C(=O)C=C] | wwww |
| 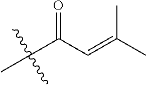 [SMILES: *C(=O)C=C(C)C] | xxxx |
| 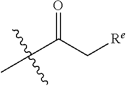 | yyyy |
| 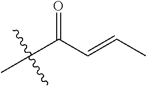 [SMILES: *C(=O)/C=C/C] | zzzz |
| 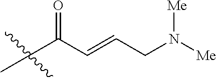 [SMILES: *C(=O)/C=C/CN(C)C] | aaaaa |
| 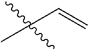 [SMILES: *C=C] | bbbbb |
| 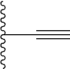 [SMILES: *C#C] | ccccc |

wherein each R^{e} is independently a suitable leaving group, NO₂, CN or oxo.

In certain embodiments, a warhead group is —C≡CH, —C≡CCH NH (isopropyl), —NHC(O)C≡CCH₂CH₃, —CH₂—C≡C≡CH₃, —C≡CCH₂OH, —CH₂C(O)C≡CH, —C(O)C≡CH, or —CH₂C(═O)C≡CH. In some embodiments, R¹ is selected from —NHC(O)CH═CH₂, —NHC(O)CH═CHCH₂N(CH₃)₂, or —CH₂NHC(O)CH—CH₂.

In certain embodiments, a warhead group is selected from those set forth in Table 3B, below, wherein each wavy line indicates the point of attachment to the rest of the molecule.

TABLE 3B
Exemplary Warhead Groups

| | |
|---|---|
| 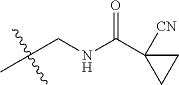 [SMILES: *CNC(=O)C1(C#N)CC1] | a |
| 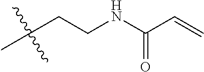 [SMILES: *CCNC(=O)C=C] | b |
| 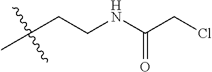 [SMILES: *CCNC(=O)CCl] | c |
| 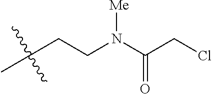 [SMILES: *CCN(C)C(=O)CCl] | d |
| 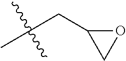 [SMILES: *CC1CO1] | e |
| 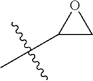 [SMILES: *C1CO1] | f |
| 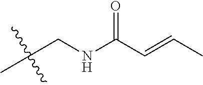 [SMILES: *CNC(=O)/C=C/C] | g |
| 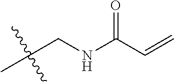 [SMILES: *CNC(=O)C=C] | h |
| 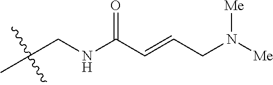 [SMILES: *CNC(=O)/C=C/CN(C)C] | i |
| 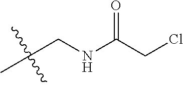 [SMILES: *CNC(=O)CCl] | j |
| 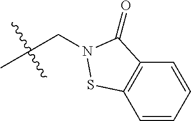 [SMILES: *Cn1sc2ccccc2c1=O] | k |
| 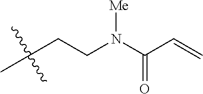 [SMILES: *CCN(C)C(=O)C=C] | l |
| 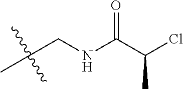 [SMILES: *CNC(=O)[C@H](C)Cl] | m |
| 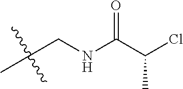 [SMILES: *CNC(=O)[C@@H](C)Cl] | n |
| 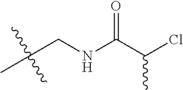 [SMILES: *CNC(=O)C(C)Cl] | o |
| 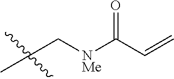 [SMILES: *CN(C)C(=O)C=C] | p |
| 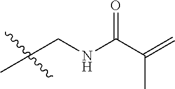 [SMILES: *CNC(=O)C(=C)C] | q |
| 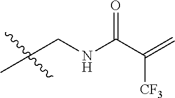 [SMILES: *CNC(=O)C(=C)C(F)(F)F] | r |
| 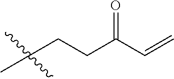 [SMILES: *CCC(=O)C=C] | s |
| 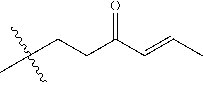 [SMILES: *CCC(=O)/C=C/C] | t |
| 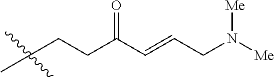 [SMILES: *CCC(=O)/C=C/CN(C)C] | u |
| 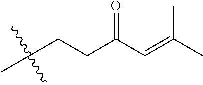 [SMILES: *CCC(=O)C=C(C)C] | v |
| 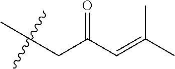 [SMILES: *CC(=O)C=C(C)C] | w |
| 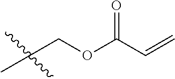 [SMILES: *COC(=O)C=C] | x |
| 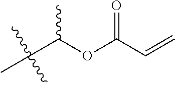 [SMILES: *C(C)OC(=O)C=C] | y |
| 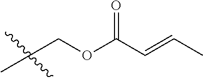 [SMILES: *COC(=O)/C=C/C] | z |
| 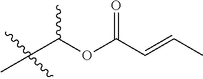 [SMILES: *C(C)OC(=O)/C=C/C] | aa |
| 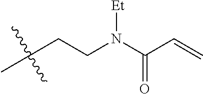 [SMILES: *CCN(CC)C(=O)C=C] | bb |
| 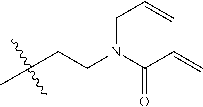 [SMILES: *CCN(CC=C)C(=O)C=C] | cc |
| 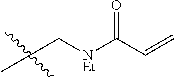 | dd |
| 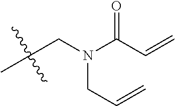 [SMILES: *CN(CC=C)C(=O)C=C] | ee |
| 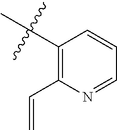 [SMILES: *c1cccnc1C=C] | ff |
| 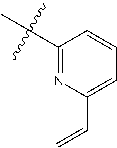 [SMILES: *c1cccc(C=C)n1] | gg |
| 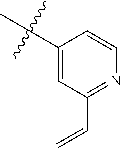 [SMILES: *c1ccnc(C=C)c1] | hh |
| 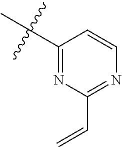 [SMILES: *c1ccnc(C=C)n1] | ii |
| 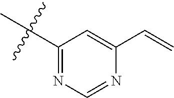 [SMILES: *c1cc(C=C)ncn1] | jj |
| 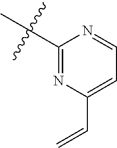 [SMILES: *c1nccc(C=C)n1] | kk |
| 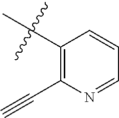 [SMILES: *c1cccnc1C#C] | ll |
| 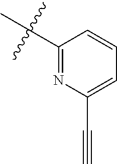 [SMILES: *c1cccc(C#C)n1] | mm |
| 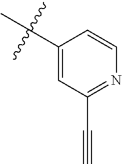 [SMILES: *c1ccnc(C#C)c1] | nn |
| 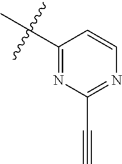 [SMILES: *c1ccnc(C#C)n1] | oo |
| 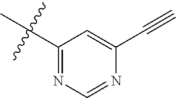 [SMILES: *c1cc(C#C)ncn1] | pp |
| 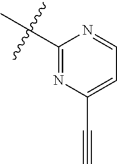 [SMILES: *c1nccc(C#C)n1] | qq |
| 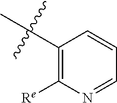 | rr |
| 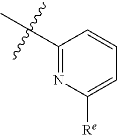 | ss |
| 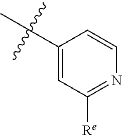 | tt |
| 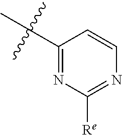 | yy |
| 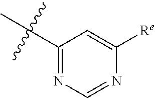 | vv |
| 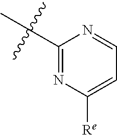 | ww |
| 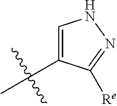 | xx |
| 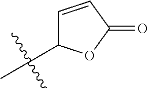 [SMILES: *C1C=CC(=O)O1] | yy |
| 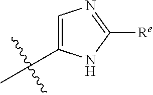 | zz |
| 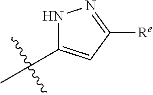 | aaa |
| 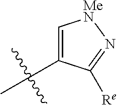 | bbb |
| 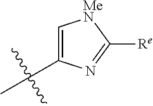 | ccc |
| 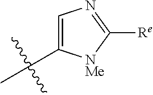 | ddd |
| 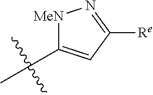 | eee |
| 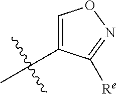 | fff |
| 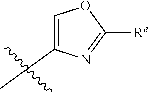 | ggg |
| 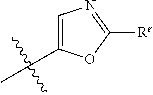 | hhh |
| 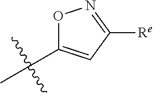 | iii |
| 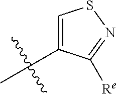 | jjj |
| 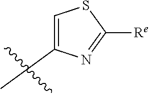 | kkk |
| 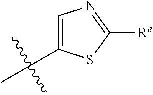 | lll |
| 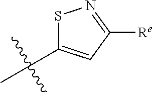 | mmm |
| 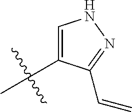 [SMILES: *c1c[nH]nc1C=C] | nnn |
| 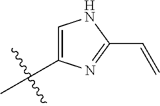 [SMILES: *c1c[nH]c(C=C)n1] | ooo |
| 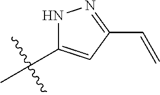 [SMILES: *c1cc(C=C)n[nH]1] | ppp |
| 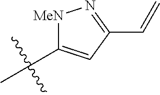 [SMILES: *c1cc(C=C)nn1C] | |
| 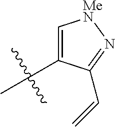 [SMILES: *c1cn(C)nc1C=C] | qqq |
| 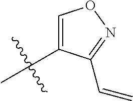 [SMILES: *c1conc1C=C] | rrr |
| 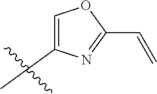 [SMILES: *c1coc(C=C)n1] | sss |
| 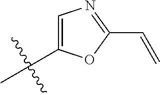 [SMILES: *c1cnc(C=C)o1] | ttt |
| 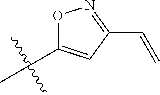 [SMILES: *c1cc(C=C)no1] | uuu |
| 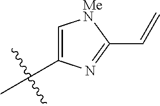 [SMILES: *c1cn(C)c(C=C)n1] | vvv |
| 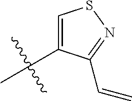 [SMILES: *c1csnc1C=C] | www |
| 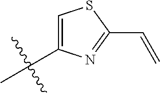 [SMILES: *c1csc(C=C)n1] | xxx |
| 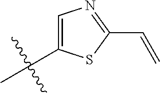 [SMILES: *c1cnc(C=C)s1] | yyy |
| 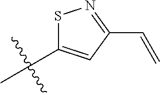 [SMILES: *c1cc(C=C)ns1] | zzz |
| 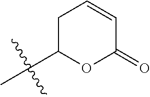 [SMILES: *C1CC=CC(=O)O1] | aaaa |
| 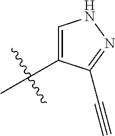 [SMILES: *c1c[nH]nc1C#C] | bbbb |
| 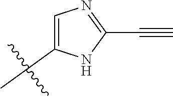 [SMILES: *c1cnc(C#C)[nH]1] | cccc |
| 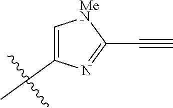 [SMILES: *c1cn(C)c(C#C)n1] | dddd |
| 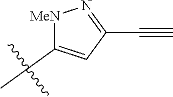 [SMILES: *c1cc(C#C)nn1C] | eeee |
| 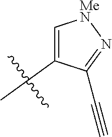 [SMILES: *c1cn(C)nc1C#C] | ffff |
| 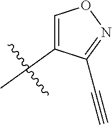 [SMILES: *c1conc1C#C] | gggg |
| 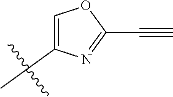 [SMILES: *c1coc(C#C)n1] | hhhh |
| 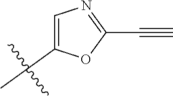 [SMILES: *c1cnc(C#C)o1] | iiii |
| 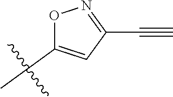 [SMILES: *c1cc(C#C)no1] | jjjj |
| 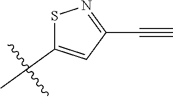 [SMILES: *c1cc(C#C)ns1] | kkkk |
| 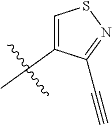 [SMILES: *c1csnc1C#C] | llll |
| 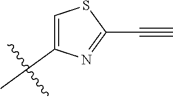 [SMILES: *c1csc(C#C)n1] | mmmm |
| 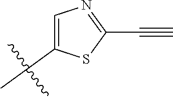 [SMILES: *c1cnc(C#C)s1] | nnnn |
| 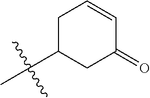 [SMILES: *C1CC=CC(=O)C1] | oooo |
| 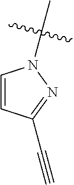 [SMILES: *n1ccc(C#C)n1] | pppp |
| 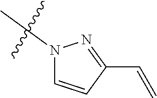 [SMILES: *n1ccc(C=C)n1] | qqqq |
| 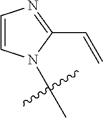 [SMILES: *n1ccnc1C=C] | rrrr |
| 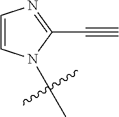 [SMILES: *n1ccnc1C#C] | ssss |
| 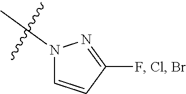 | tttt |
| 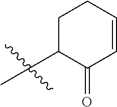 [SMILES: *C1CCC=CC1=O] | uuuu |
| 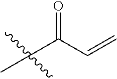 [SMILES: *C(=O)C=C] | vvvv |
| 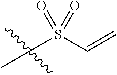 [SMILES: *S(=O)(=O)C=C] | wwww |
| 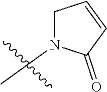 [SMILES: *N1CC=CC1=O] | xxxx |
| 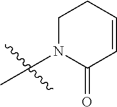 [SMILES: *N1CCC=CC1=O] | yyyy |
| 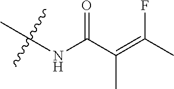 [SMILES: *NC(=O)/C(C)=C(/C)F] | zzzz |
| 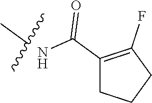 | aaaaa |
| 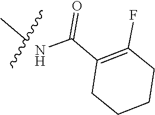 | bbbbb |
| 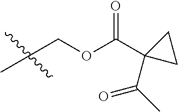 [SMILES: *COC(=O)C1(C(C)=O)CC1] | ccccc |
| 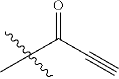 [SMILES: *C(=O)C#C] | ddddd |
| 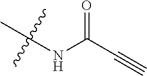 [SMILES: *NC(=O)C#C] | eeeee |
| 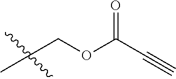 [SMILES: *COC(=O)C#C] | fffff |
| 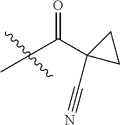 [SMILES: *C(=O)C1(C#N)CC1] | ggggg |
| 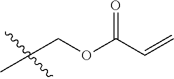 [SMILES: *COC(=O)C=C] | hhhhh |
| 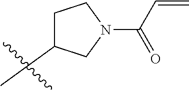 [SMILES: *C1CCN(C(=O)C=C)C1] | iiiii |
| 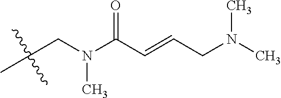 [SMILES: *CN(C)C(=O)/C=C/CN(C)C] | jjjjj |
| 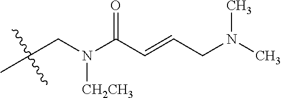 [SMILES: *CN(CC)C(=O)/C=C/CN(C)C] | kkkkk |
| 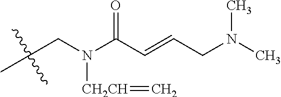 | lllll |
| 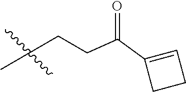 [SMILES: *CCC(=O)C1=CCC1] | mmmmm |
| 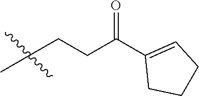 [SMILES: *CCC(=O)C1=CCCC1] | nnnnn |
| 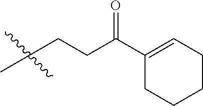 [SMILES: *CCC(=O)C1=CCCCC1] | ooooo |
| 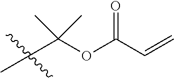 [SMILES: *C(C)(C)OC(=O)C=C] | ppppp |
| 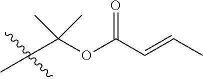 [SMILES: *C(C)(C)OC(=O)/C=C/C] | qqqqq |
| 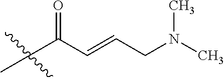 [SMILES: *C(=O)/C=C/CN(C)C] | rrrrr |
| 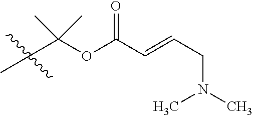 [SMILES: *C(C)(C)OC(=O)/C=C/CN(C)C] | sssss |
| 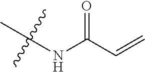 [SMILES: *NC(=O)C=C] | ttttt |
| 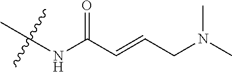 [SMILES: *NC(=O)/C=C/CN(C)C] | uuuuu |
| 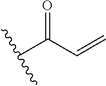 [SMILES: *C(=O)C=C] | vvvvv |
| 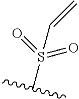 [SMILES: *S(=O)(=O)C=C] | wwwww |
| 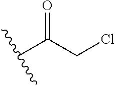 [SMILES: *C(=O)CCl] | xxxxx |
| 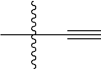 [SMILES: *C#C] | yyyyy |
| 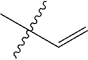 [SMILES: *C=C] | zzzzz |
| 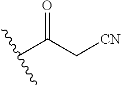 [SMILES: *C(=O)CC#N] | aaaaaa |
| 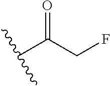 [SMILES: *C(=O)CF] | bbbbbb |
| 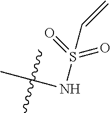 [SMILES: *NS(=O)(=O)C=C] | cccccc |
| 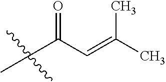 [SMILES: *C(=O)C=C(C)C] | dddddd |
| 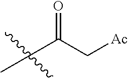 [SMILES: *C(=O)CC(C)=O] | eeeeee |
| 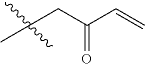 [SMILES: *CC(=O)C=C] | ffffff |
| 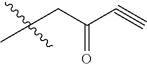 [SMILES: *CC(=O)C#C] | gggggg |
| 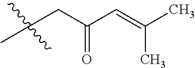 [SMILES: *CC(=O)C=C(C)C] | hhhhhh |
| 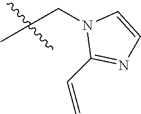 [SMILES: *Cn1ccnc1C=C] | iiiiii |
| 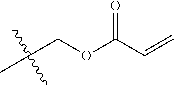 [SMILES: *COC(=O)C=C] | jjjjjj |
| 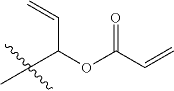 [SMILES: *C(C=C)OC(=O)C=C] | kkkkkk |
| 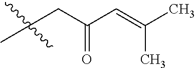 [SMILES: *CC(=O)C=C(C)C] | llllll |
| 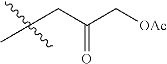 [SMILES: *CC(=O)COC(C)=O] | mmmmmm |
| 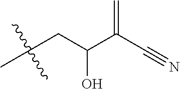 [SMILES: *CC(O)C(=C)C#N] | nnnnnn |
| 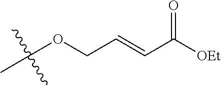 [SMILES: *OC/C=C/C(=O)OCC] | oooooo |
| 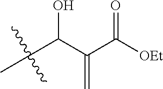 [SMILES: *C(O)C(=C)C(=O)OCC] | pppppp |
| 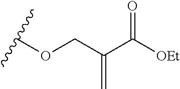 [SMILES: *OCC(=C)C(=O)OCC] | qqqqqq |
| 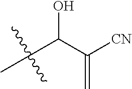 [SMILES: *C(O)C(=C)C#N] | rrrrrr |
| 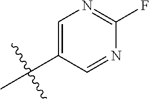 [SMILES: *c1cnc(F)nc1] | ssssss |
| 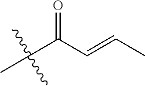 [SMILES: *C(=O)/C=C/C] | tttttt |
| 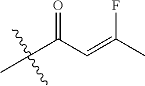 [SMILES: *C(=O)/C=C(/C)F] | uuuuuu |
| 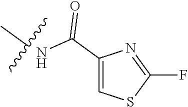 | vvvvvv |
| 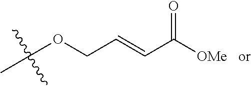 [SMILES: *OC/C=C/C(=O)OC] | wwwwww |
| 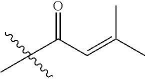 [SMILES: *C(=O)C=C(C)C] | xxxxxx |

wherein each R^{e} is independently a suitable leaving group, NO₂, CN, or oxo.

In some embodiments, Y of a warhead group is an isoxazoline compound or derivative capable of covalently binding to serine. In some embodiments, Y of a warhead group is an isoxazoline compound or derivative described in WO 2010135360, the entire content of which is incorporated herein by reference. As understood by one skilled in the art, an isoxazoline compound or derivative described in WO 2010135360, as Y of a warhead group, can covalently connect to L² of the warhead group at any reasonable position of the isoxazoline compound or derivative. In some embodiments, Y of a warhead group is:

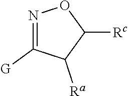

wherein G, R^{a}, and R^{c} are:

| G | R^{a} | R^{c} |
|---|---|---|
| —Br | —H | —H |
| —Cl | —H | —H |
| 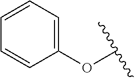 [SMILES: *Oc1ccccc1] | —H | —H |
| 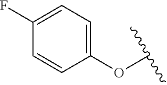 [SMILES: *Oc1ccc(F)cc1] | —H | —H |
| 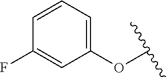 [SMILES: *Oc1cccc(F)c1] | —H | —H |
| 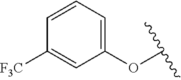 [SMILES: *Oc1cccc(C(F)(F)F)c1] | —H | —H |
| 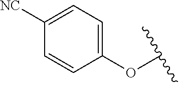 [SMILES: *Oc1ccc(C#N)cc1] | —H | —H |
| 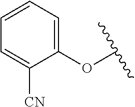 [SMILES: *Oc1ccccc1C#N] | —H | —H |
| 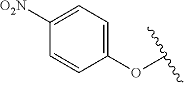 [SMILES: *Oc1ccc([N+](=O)[O-])cc1] | —H | —H |
| 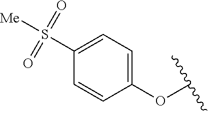 [SMILES: *Oc1ccc(S(C)(=O)=O)cc1] | —H | —H |
| 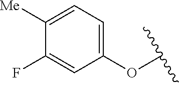 [SMILES: *Oc1ccc(C)c(F)c1] | —H | —H |
| 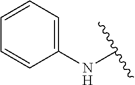 [SMILES: *Nc1ccccc1] | —H | —H |
| 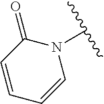 [SMILES: *n1ccccc1=O] | —H | —H |
| 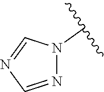 [SMILES: *n1cncn1] | —H | —H |
| 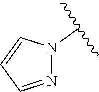 [SMILES: *n1cccn1] | —H | —H |
| 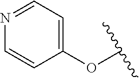 [SMILES: *Oc1ccncc1] | —H | —H |
| 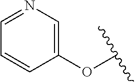 [SMILES: *Oc1cccnc1] | —H | —H |
| 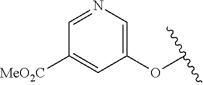 [SMILES: *Oc1cncc(C(=O)OC)c1] | —H | —H |
| 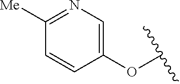 [SMILES: *Oc1ccc(C)nc1] | —H | —H |
| 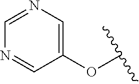 [SMILES: *Oc1cncnc1] | —H | —H |
| 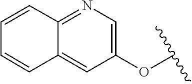 [SMILES: *Oc1cnc2ccccc2c1] | —H | —H |
| 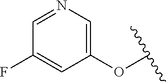 [SMILES: *Oc1cncc(F)c1] | —H | —H |
| 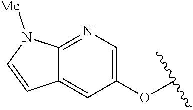 [SMILES: *Oc1cnc2c(ccn2C)c1] | —H | —H |
| —OMe | —H | —H |
| 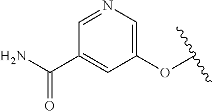 [SMILES: *Oc1cncc(C(N)=O)c1] | —H | —H |
| 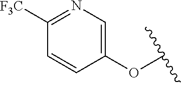 [SMILES: *Oc1ccc(C(F)(F)F)nc1] | —H | —H |
| 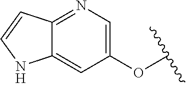 [SMILES: *Oc1cnc2cc[nH]c2c1] | —H | —H |
| 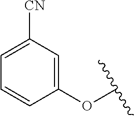 [SMILES: *Oc1cccc(C#N)c1] | —H | —H |
| 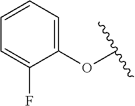 [SMILES: *Oc1ccccc1F] | —H | —H |
| 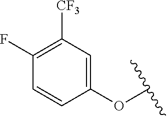 [SMILES: *Oc1ccc(F)c(C(F)(F)F)c1] | —H | —H |
| 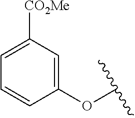 [SMILES: *Oc1cccc(C(=O)OC)c1] | —H | —H |
| 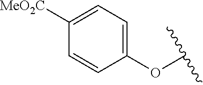 [SMILES: *Oc1ccc(C(=O)OC)cc1] | —H | —H |
| 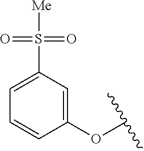 [SMILES: *Oc1cccc(S(C)(=O)=O)c1] | —H | —H |
| 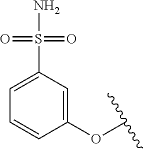 [SMILES: *Oc1cccc(S(N)(=O)=O)c1] | —H | —H |
| 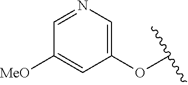 [SMILES: *Oc1cncc(OC)c1] | —H | —H |
| 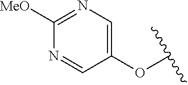 [SMILES: *Oc1cnc(OC)nc1] | —H | —H |
| 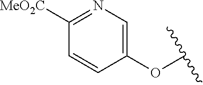 [SMILES: *Oc1ccc(C(=O)OC)nc1] | —H | —H |
| 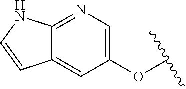 [SMILES: *Oc1cnc2[nH]ccc2c1] | —H | —H |
| 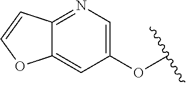 [SMILES: *Oc1cnc2ccoc2c1] | —H | —H |
| 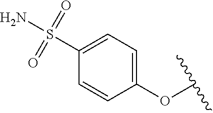 [SMILES: *Oc1ccc(S(N)(=O)=O)cc1] | —H | —H |
| 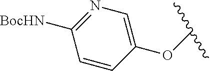 [SMILES: *Oc1ccc(NC(=O)OC(C)(C)C)nc1] | —H | —H |
| 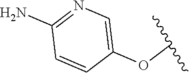 [SMILES: *Oc1ccc(N)nc1] | —H | —H |
| 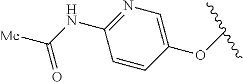 [SMILES: *Oc1ccc(NC(C)=O)nc1] | —H | —H |
| 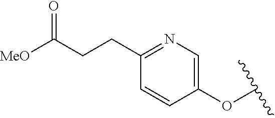 [SMILES: *Oc1ccc(CCC(=O)OC)nc1] | —H | —H |
| 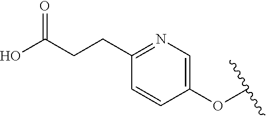 [SMILES: *Oc1ccc(CCC(=O)O)nc1] | —H | —H |
| 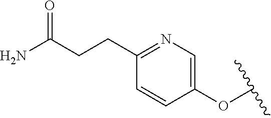 [SMILES: *Oc1ccc(CCC(N)=O)nc1] | —H | —H |
| 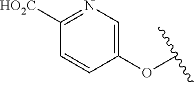 [SMILES: *Oc1ccc(C(=O)O)nc1] | —H | —H |
| 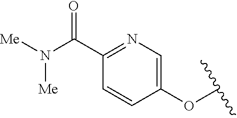 [SMILES: *Oc1ccc(C(=O)N(C)C)nc1] | —H | —H |
| 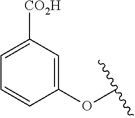 [SMILES: *Oc1cccc(C(=O)O)c1] | —H | —H |
| 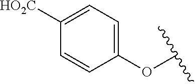 [SMILES: *Oc1ccc(C(=O)O)cc1] | —H | —H |
| 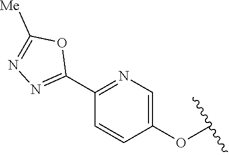 [SMILES: *Oc1ccc(-c2nnc(C)o2)nc1] | —H | —H |
| 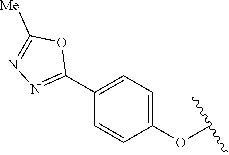 [SMILES: *Oc1ccc(-c2nnc(C)o2)cc1] | —H | —H |
| 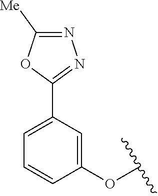 [SMILES: *Oc1cccc(-c2nnc(C)o2)c1] | —H | —H |
| 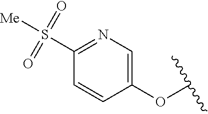 [SMILES: *Oc1ccc(S(C)(=O)=O)nc1] | —H | —H |
| 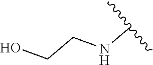 [SMILES: *NCCO] | —H | —H |
| 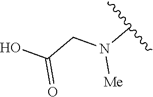 [SMILES: *N(C)CC(=O)O] | —H | —H |
| 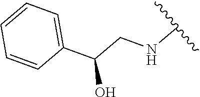 [SMILES: *NC[C@@H](O)c1ccccc1] | —H | —H |
| 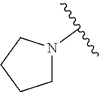 [SMILES: *N1CCCC1] | —H | —H |
| 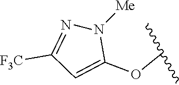 [SMILES: *Oc1cc(C(F)(F)F)nn1C] | —H | —H |
| 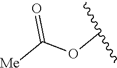 [SMILES: *OC(C)=O] | —H | —H |
| —Br | —CH₃ | —H |
| —Br | —CH₃ | —H |
| 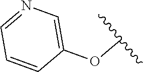 [SMILES: *Oc1cccnc1] | —CH₃ | —H |
| —Br | —H | —CH₃ |
| 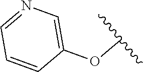 [SMILES: *Oc1cccnc1] | —H | —CH₃ |
| —Br | —H | —CF₃ |
| 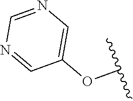 [SMILES: *Oc1cncnc1] | —H | —CF₃ |
| —Br | —H | —CH₂CH₃ |

### Lysine Mimetic

In some embodiments, DIM is LBM as described above and herein. In some embodiments, DIM is a lysine mimetic. In some embodiments, the covalent attachment of ubiquitin to BCL-XL protein is achieved through the action of a lysine mimetic. In some embodiments, upon the binding of a compound of formula I to BCL-XL, the DIM moiety that mimics a lysine undergoes ubiquitination thereby marking BCL-XL for degradation via the Ubiquitin-Proteasome Pathway (UPP).

In some embodiments, DIM is

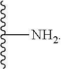 [SMILES: *N]

In some embodiments, DIM is

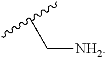 [SMILES: *CN]

In some embodiments, DIM is

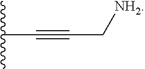 [SMILES: *C#CCN]

In some embodiments, DIM is selected from those depicted in Table 1, below.

In some embodiments, the present invention provides the compound of formula I as a compound of formula I-p-1:

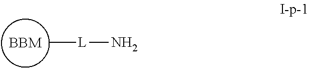

or a pharmaceutically acceptable salt thereof, wherein each of BBM and L is as defined above and described in embodiments herein, both singly and in combination.

In some embodiments, the present invention provides the compound of formula I as a compound of formula I-p-2:

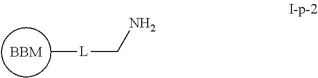

or a pharmaceutically acceptable salt thereof, wherein each of BBM and L is as defined above and described in embodiments herein, both singly and in combination.

In some embodiments, the present invention provides the compound of formula I as a compound of formula I-p-3:

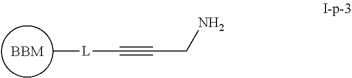

or a pharmaceutically acceptable salt thereof, wherein each of BBM and L is as defined above and described in embodiments herein, both singly and in combination.

In certain embodiments, the present invention provides a compound of formula I, wherein DIM is a lysine mimetic

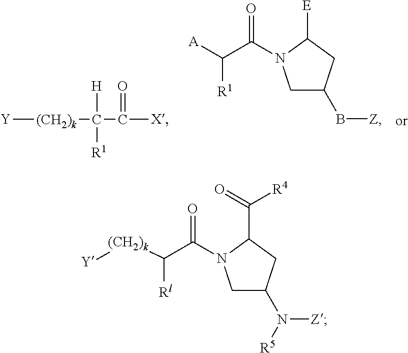

thereby forming a compound of Formulae I-q-1. I-q-2, or I-q-3, respectively:

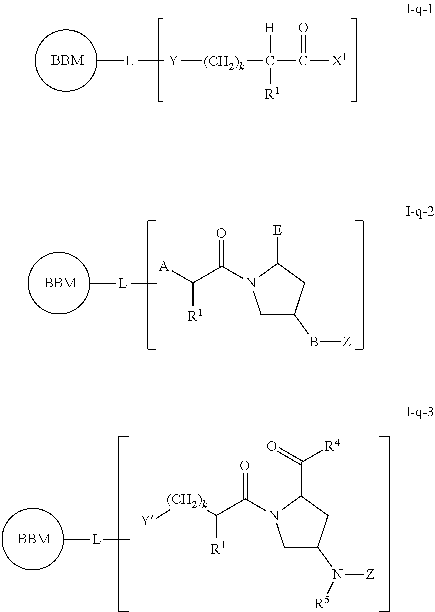

or a pharmaceutically acceptable salt thereof, wherein L and BBM are as defined above and described in embodiments herein, and wherein each of the variables R¹, R⁴, R⁵, A, B, E, Y, Y′, Z, Z′, and k are as defined and described in U.S. Pat. No. 7,622,496, the entirety of each of which is herein incorporated by reference.

### Hydrogen Atom

In some embodiments, DIM is a hydrogen atom. In some embodiments, the covalent attachment of ubiquitin to BCL-XL protein is achieved through a provided compound wherein DIM is a hydrogen atom. In some embodiments, upon the binding of a compound of formula I to BCL-XL, the DIM moiety being hydrogen effectuates ubiquitination thereby marking BCL-XL for degradation via the Ubiquitin-Proteasome Pathway (UPP).

In some embodiments, DIM is selected from those depicted in Table 1, below.

In some embodiments, the present invention provides the compound of formula I wherein DIM is a hydrogen atom, thereby forming a compound of formula I-r:

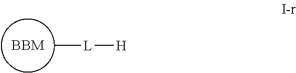

or a pharmaceutically acceptable salt thereof, wherein each of BBM and L is as defined above and described in embodiments herein, both singly and in combination.

### Linker (L)

As defined above and described herein, L is a bivalent moiety that connects to BBM to DIM.

In some embodiments, L is a bivalent moiety that connects BBM to DIM. In some embodiments, L is a bivalent moiety that connects BBM to LBM. In some embodiments, L is a bivalent moiety that connects BBM to a lysine mimetic.

In some embodiments, L is a covalent bond or a bivalent, saturated or partially unsaturated, straight or branched C₁₋₅₀ hydrocarbon chain, wherein 0-10 methylene units of L are independently replaced by —C(D)(H)—, —C(D)₂-, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

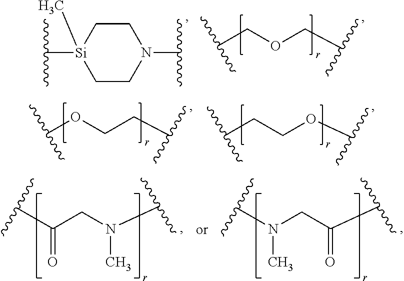

wherein each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur, wherein r is 0, 1, 2, 3, 4, 5, 6, 7, 8, 9, or 10, and wherein R is as defined and described herein.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 4-11 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 4-11 membered saturated or partially unsaturated spiro heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, -Cy- is

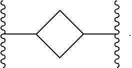 [SMILES: *C1CC(*)C1]

In some embodiments, -Cy- is

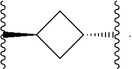 [SMILES: *[C@H]1C[C@H](*)C1]

In some embodiments, -Cy- is

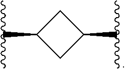 [SMILES: *[C@H]1C[C@@H](*)C1]

In some embodiments, -Cy- is

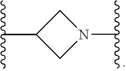 [SMILES: *C1CN(*)C1]

In some embodiments, -Cy- is

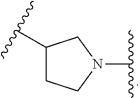 [SMILES: *C1CCN(*)C1]

In some embodiments, -Cy- is

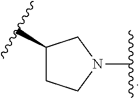 [SMILES: *[C@@H]1CCN(*)C1]

In some embodiments, -Cy- is

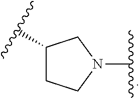 [SMILES: *[C@H]1CCN(*)C1]

In some embodiments, -Cy- is

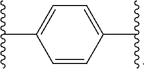 [SMILES: *c1ccc(*)cc1]

In some embodiments, -Cy- is

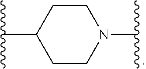 [SMILES: *C1CCN(*)CC1]

In some embodiments, -Cy- is

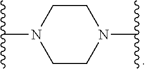 [SMILES: *N1CCN(*)CC1]

In some embodiments, -Cy- is

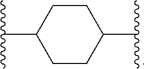 [SMILES: *C1CCC(*)CC1]

In some embodiments, -Cy- is

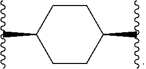 [SMILES: *[C@H]1CC[C@@H](*)CC1]

In some embodiments, -Cy- is

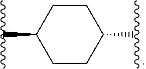 [SMILES: *[C@H]1CC[C@H](*)CC1]

In some embodiments, -Cy- is

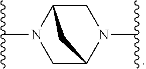 [SMILES: *N1C[C@@H]2C[C@H]1CN2*]

In some embodiments, -Cy- is

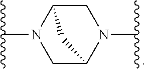 [SMILES: *N1C[C@H]2C[C@@H]1CN2*]

In some embodiments, -Cy- is

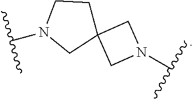 [SMILES: *N1CCC2(C1)CN(*)C2]

In some embodiments, -Cy- is

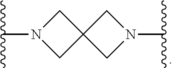 [SMILES: *N1CC2(C1)CN(*)C2]

In some embodiments, -Cy- is

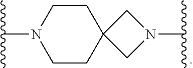 [SMILES: *N1CCC2(CC1)CN(*)C2]

In some embodiments, -Cy- is

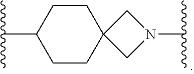 [SMILES: *C1CCC2(CC1)CN(*)C2]

In some embodiments, -Cy- is

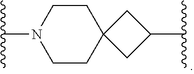 [SMILES: *C1CC2(CCN(*)CC2)C1]

In some embodiments, -Cy- is

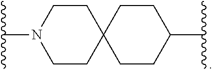 [SMILES: *C1CCC2(CC1)CCN(*)CC2]

In some embodiments, -Cy- is

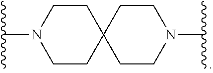 [SMILES: *N1CCC2(CC1)CCN(*)CC2]

In some embodiments, -Cy- is selected from those as depicted in the compounds of Table 1, below.

In some embodiments, L is —NR—(C₁₋₁₀ aliphatic)-. In some embodiments, L is —(C₁₋₁₀ aliphatic)-NR—(C₁₋₁₀aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀ aliphatic)-. In some embodiments, L is —(C₁₋₁₀ aliphatic)-CONR—(CH₂CH₂O)₁₋₁₀CH₂CH₂—. In some embodiments, L is -Cy-CONR—(C₁₋₁₀ aliphatic)-. In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy-CONR—(C₁₋₁₀ aliphatic)-. In some embodiments, L is -Cy-(C₁₋₁₀ aliphatic)-CONR-Cy-(C₁₋₁₀ aliphatic)-.

In some embodiments, L is —NRCO—(C₁₋₁₀ aliphatic)-. In some embodiments, L is —(C₁₋₁₀ aliphatic)-NRCO—(C₁₋₁₀aliphatic)-. In some embodiments, L is —(C₁₋₁₀ aliphatic)-NRCO—(CH₂CH₂O)₁₋₁₀CH₂CH₂—. In some embodiments, L is -Cy-NRCO—(C₁₋₁₀ aliphatic)-. In some embodiments, L is -Cy-(C₁₋₁₀ aliphatic)-NRCO—. In some embodiments, L is -Cy-(C₁₋₁₀ aliphatic)-NRCO—(C₁₋₁₀ aliphatic)-. In some embodiments, L is —(C₁₋₁₀ aliphatic)-Cy-NRCO—(C₁₋₁₀ aliphatic)-. In some embodiments, L is —(C₁₋₁₀ aliphatic)-Cy-(C₁₋₁₀ aliphatic)-NRCO—. In some embodiments, L is —(C₁₋₁₀ aliphatic)-Cy-(C₁₋₁₀ aliphatic)-NRCO—(C₁₋₁₀ aliphatic)-. In some embodiments, L is -Cy-(C₁₋₁₀ aliphatic)-Cy-NRCO—. In some embodiments, L is -Cy-(C₁₋₁₀ aliphatic)-NRCO-Cy-. In some embodiments, L is -Cy-(C₁₋₁₀ aliphatic)-Cy-NRCO—(C₁₋₁₀ aliphatic)-. In some embodiments, L is -Cy-(C₁₋₁₀ aliphatic)-NRCO-Cy-(C₁₋₁₀ aliphatic)-.

In some embodiments, L is —O—(C₁₋₁₀ aliphatic)-. In some embodiments, L is —(C₁₋₁₀ aliphatic)-O—(C₁₋₁₀ aliphatic)-. In some embodiments, L is —(C₁₋₁₀ aliphatic)-O—(CH₂CH₂O)₁₋₁₀CH₂CH₂—. In some embodiments, L is -Cy-O—(C₁₋₁₀ aliphatic)-. In some embodiments, L is -Cy-(C₁₋₁₀ aliphatic)-O—. In some embodiments, L is -Cy-(C₁₋₁₀ aliphatic)-O—(C₁₋₁₀ aliphatic)-. In some embodiments, L is —(C₁₋₁₀ aliphatic)-Cy-O—(C₁₋₁₀ aliphatic)-. In some embodiments, L is —(C₁₋₁₀ aliphatic)-Cy-(C₁₋₁₀ aliphatic)-O—. In some embodiments, L is —(C₁₋₁₀ aliphatic)-Cy-(C₁₋₁₀ aliphatic)-O—(C₁₋₁₀ aliphatic)-. In some embodiments, L is -Cy-(C₁₋₁₀ aliphatic)-Cy-O—. In some embodiments, L is -Cy-(C₁₋₁₀ aliphatic)-O-Cy-. In some embodiments, L is -Cy-(C₁₋₁₀ aliphatic)-Cy-O—(C₁₋₁₀ aliphatic)-. In some embodiments, L is -Cy-(C₁₋₁₀ aliphatic)-O-Cy-(C₁₋₁₀ aliphatic)-.

In some embodiments, L is -Cy-(C₁₋₁₀aliphatic)-. In some embodiments, L is —(C₁₋₁₀ aliphatic)-Cy-(C₁₋₁₀ aliphatic)-. In some embodiments, L is —(C₁₋₁₀ aliphatic)-Cy-(CH₂CH₂O)₁₋₁₀CH₂CH₂—. In some embodiments, L is -Cy-(C₁₋₁₀ aliphatic)-Cy-. In some embodiments, L is -Cy-(C₁₋₁₀ aliphatic)-Cy-(C₁₋₁₀ aliphatic)-. In some embodiments, L is -Cy-(C₁₋₁₀ aliphatic)-Cy-(C₁₋₁₀ aliphatic)-Cy-. In some embodiments, L is —(C₁₋₁₀ aliphatic)-Cy-(C₁₋₁₀ aliphatic)-Cy-(C₁₋₁₀ aliphatic)-.

In some embodiments, L is —NR—(CH₂)₁₋₁₀—. In some embodiments, L is —(CH₂)₁₋₁₀—NR—(CH₂)₁₋₁₀—. In some embodiments, L is —(CH₂)₁₋₁₀—NR—(CH₂CH₂O)₁₋₁₀CH₂CH₂—. In some embodiments, L is -Cy-NR—(CH₂)₁₋₁₀—. In some embodiments, L is -Cy-(CH₂)₁₋₁₀—NR—. In some embodiments, L is -Cy-(CH₂)₁₋₁₀—NR—(CH₂)₁₋₁₀—. In some embodiments, L is —(CH₂)₁₋₁₀-Cy-NR—(CH₂)₁₋₁₀—. In some embodiments, L is —(CH₂)₁₋₁₀-Cy-(CH₂)₁₋₁₀—NR—. In some embodiments, L is —(CH₂)₁₋₁₀-Cy-(CH₂)₁₋₁₀—NR—(CH₂)₁₋₁₀—. In some embodiments, L is -Cy-(CH₂)₁₋₁₀-Cy-NR—. In some embodiments, L is -Cy-(CH₂)₁₋₁₀—NR-Cy-. In some embodiments, L is -Cy-(CH₂)₁₋₁₀-Cy-NR—(CH₂)₁₋₁₀—. In some embodiments, L is -Cy-(CH₂)₁₋₁₀—NR-Cy-(CH₂)₁₋₁₀—.

In some embodiments, L is —CONR—(CH₂)₁₋₁₀—. In some embodiments, L is —(CH₂)₁₋₁₀—CONR—(CH₂)₁₋₁₀—. In some embodiments, L is —(CH₂)₁₋₁₀—CONR—(CH₂CH₂O)₁₋₁₀CH₂CH₂—. In some embodiments, L is -Cy-CONR—(CH₂)₁₋₁₀—. In some embodiments, L is -Cy-(CH₂)₁₋₁₀—CONR—. In some embodiments, L is -Cy-(CH₂)₁₋₁₀—CONR—(CH₂)₁₋₁₀—. In some embodiments, L is —(CH₂)₁₋₁₀-Cy-CONR—(CH₂)₁₋₁₀—. In some embodiments, L is —(CH₂)₁₋₁₀-Cy-(CH₂)₁₋₁₀—CONR—. In some embodiments, L is —(CH₂)₁₋₁₀-Cy-(CH₂)₁₋₁₀—CONR—(CH₂)₁₋₁₀—. In some embodiments, L is -Cy-(CH₂)₁₋₁₀-Cy-CONR—. In some embodiments, L is -Cy-(CH₂)₁₋₁₀—CONR-Cy-. In some embodiments, L is -Cy-(CH₂)₁₋₁₀-Cy-CONR—(CH₂)₁₋₁₀—. In some embodiments, L is -Cy-(CH₂)₁₋₁₀—CONR-Cy-(CH₂)₁₋₁₀—.

In some embodiments, L is —NRCO—(CH₂)₁₋₁₀—. In some embodiments, L is —(CH₂)₁₋₁₀—NRCO—(CH₂)₁₋₁₀—. In some embodiments, L is —(CH₂)₁₋₁₀—NRCO—(CH₂CH₂O)₁₋₁₀CH₂CH₂—. In some embodiments, L is -Cy-NRCO—(CH₂)₁₋₁₀—. In some embodiments, L is -Cy-(CH₂)₁₋₁₀—NRCO—. In some embodiments, L is -Cy-(CH₂)₁₋₁₀—NRCO—(CH₂)₁₋₁₀—. In some embodiments, L is —(CH₂)₁₋₁₀-Cy-NRCO—(CH₂)₁₋₁₀—. In some embodiments, L is —(CH₂)₁₋₁₀-Cy-(CH₂)₁₋₁₀—NRCO—. In some embodiments, L is —(CH₂)₁₋₁₀-Cy-(CH₂)₁₋₁₀—NRCO—(CH₂)₁₋₁₀—. In some embodiments, L is -Cy-(CH₂)₁₋₁₀-Cy-NRCO—. In some embodiments, L is -Cy-(CH₂)₁₋₁₀—NRCO-Cy-. In some embodiments, L is -Cy-(CH₂)₁₋₁₀-Cy-NRCO—(CH₂)₁₋₁₀—. In some embodiments, L is -Cy-(CH₂)₁₋₁₀—NRCO-Cy-(CH₂)₁₋₁₀—.

In some embodiments, L is —O—(CH₂)₁₋₁₀—. In some embodiments, L is —(CH₂)₁₋₁₀—O—(CH₂)₁₋₁₀—. In some embodiments, L is —(CH₂)₁₋₁₀—O—(CH₂CH₂O)₁₋₁₀CH₂CH₂—. In some embodiments, L is -Cy-O—(CH₂)₁₋₁₀—. In some embodiments, L is -Cy-(CH₂)₁₋₁₀—O—. In some embodiments, L is -Cy-(CH₂)₁₋₁₀—O—(CH₂)₁₋₁₀—. In some embodiments, L is —(CH₂)₁₋₁₀-Cy-O—(CH₂)₁₋₁₀—. In some embodiments, L is —(CH₂)₁₋₁₀—Cy-(CH₂)₁₋₁₀—O—. In some embodiments, L is —(CH₂)₁₋₁₀-Cy-(CH₂)₁₋₁₀—O—(CH₂)₁₋₁₀—. In some embodiments, L is -Cy-(CH₂)₁₋₁₀-Cy-O—. In some embodiments, L is -Cy-(CH₂)₁₋₁₀—O-Cy-. In some embodiments, L is -Cy-(CH₂)₁₋₁₀-Cy-O—(CH₂)₁₋₁₀—. In some embodiments, L is -Cy-(CH₂)₁₋₁₀—O-Cy-(CH₂)₁₋₁₀—.

In some embodiments, L is -Cy-(CH₂)₁₋₁₀—. In some embodiments, L is —(CH₂)₁₋₁₀-Cy-(CH₂)₁₋₁₀—. In some embodiments, L is —(CH₂)₁₋₁₀-Cy-(CH₂CH₂O)₁₋₁₀CH₂CH₂—. In some embodiments, L is -Cy-(CH₂)₁₋₁₀-Cy-. In some embodiments, L is -Cy-(CH₂)₁₋₁₀-Cy-(CH₂)₁₋₁₀—. In some embodiments, L is -Cy-(CH₂)₁₋₁₀-Cy-(CH₂)₁₋₁₀-Cy-. In some embodiments, L is —(CH₂)₁₋₁₀-Cy-(CH₂)₁₋₁₀-Cy-(CH₂)₁₋₁₀—.

In some embodiments, L is

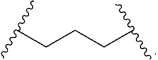 [SMILES: *CCC*]

In some embodiments, L is

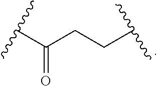 [SMILES: *CCC(*)=O]

In some embodiments, L is

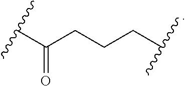 [SMILES: *CCCC(*)=O]

In some embodiments, L is

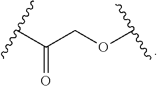 [SMILES: *OCC(*)=O]

In some embodiments, L is

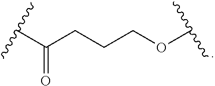 [SMILES: *OCCCC(*)=O]

In some embodiments, L is

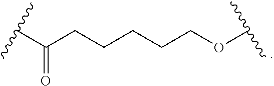 [SMILES: *OCCCCCC(*)=O]

In some embodiments, L is

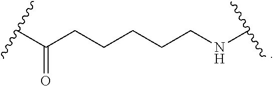 [SMILES: *NCCCCCC(*)=O]

In some embodiments, L is

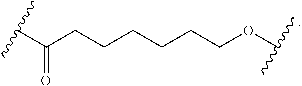 [SMILES: *OCCCCCCC(*)=O]

In some embodiments, L is

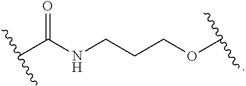 [SMILES: *OCCCNC(*)=O]

In some embodiments, L is

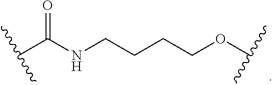 [SMILES: *OCCCCNC(*)=O]

In some embodiments, L is

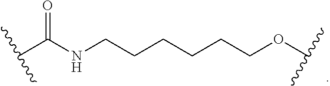 [SMILES: *OCCCCCCNC(*)=O]

In some embodiments, L is

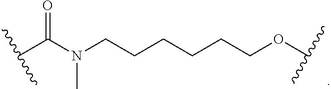 [SMILES: *OCCCCCCN(C)C(*)=O]

In some embodiments, L is

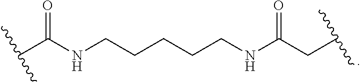 [SMILES: *CC(=O)NCCCCCNC(*)=O]

In some embodiments, L is

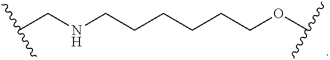 [SMILES: *CNCCCCCCO*]

In some embodiments, L is

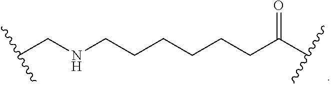 [SMILES: *CNCCCCCCC(*)=O]

In some embodiments, L is

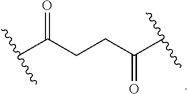 [SMILES: *C(=O)CCC(*)=O]

In some embodiments, L is

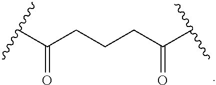 [SMILES: *C(=O)CCCC(*)=O]

In some embodiments, L is

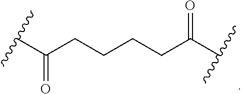 [SMILES: *C(=O)CCCCC(*)=O]

In some embodiments, L is

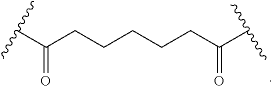 [SMILES: *C(=O)CCCCCC(*)=O]

In some embodiments, L is

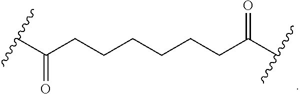 [SMILES: *C(=O)CCCCCCC(*)=O]

In some embodiments, L is

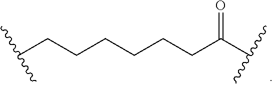 [SMILES: *CCCCCCC(*)=O]

In some embodiments, L is

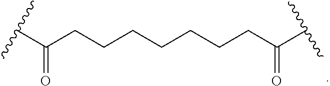 [SMILES: *C(=O)CCCCCCCC(*)=O]

In some embodiments, L is

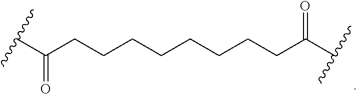 [SMILES: *C(=O)CCCCCCCCC(*)=O]

In some embodiments, L is

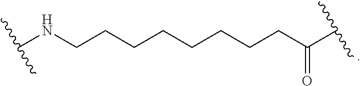 [SMILES: *NCCCCCCCCC(*)=O ; "."]

In some embodiments, L is

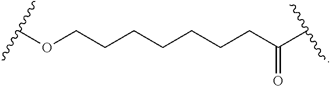 [SMILES: *OCCCCCCCC(*)=O]

In some embodiments, L is

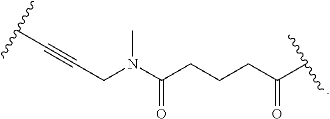 [SMILES: *C#CCN(C)C(=O)CCCC(*)=O]

In some embodiments, L is

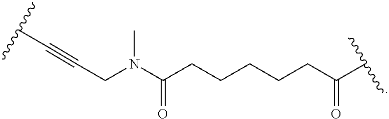 [SMILES: *C#CCN(C)C(=O)CCCCCC(*)=O]

In some embodiments, L is

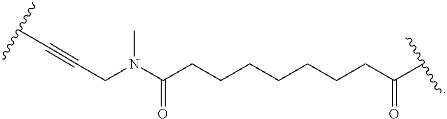 [SMILES: *C#CCN(C)C(=O)CCCCCCCC(*)=O]

In some embodiments, L is

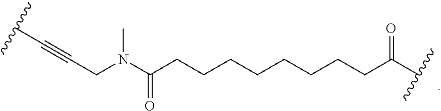 [SMILES: *C#CCN(C)C(=O)CCCCCCCCC(*)=O]

In some embodiments, L is

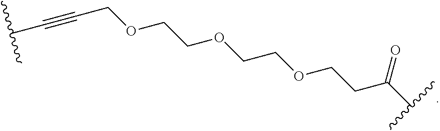 [SMILES: *C#CCOCCOCCOCCC(*)=O]

In some embodiments, L is

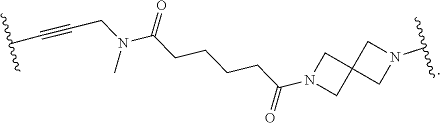 [SMILES: *C#CCN(C)C(=O)CCCCC(=O)N1CC2(CN(*)C2)C1]

In some embodiments, L is

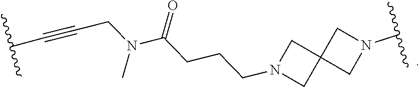 [SMILES: *C#CCN(C)C(=O)CCCN1CC2(CN(*)C2)C1]

In some embodiments, L is

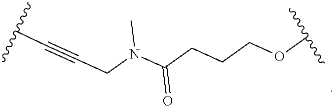 [SMILES: *C#CCN(C)C(=O)CCCO*]

In some embodiments, L is

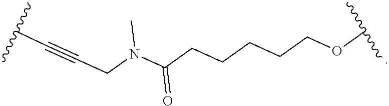 [SMILES: *C#CCN(C)C(=O)CCCCCO*]

In some embodiments, L is

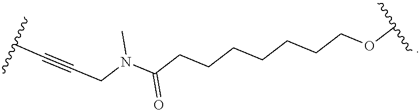 [SMILES: *C#CCN(C)C(=O)CCCCCCCO*]

In some embodiments, L is

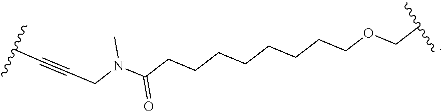 [SMILES: *C#CCN(C)C(=O)CCCCCCCCOC*]

In some embodiments, L is

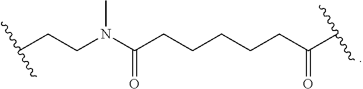 [SMILES: *CCN(C)C(=O)CCCCCC(*)=O]

In some embodiments, L is

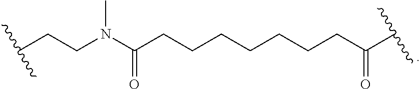 [SMILES: *CCN(C)C(=O)CCCCCCCC(*)=O]

In some embodiments, L is

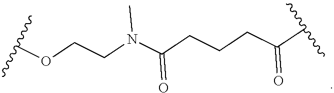 [SMILES: *OCCN(C)C(=O)CCCC(*)=O]

In some embodiments, L is

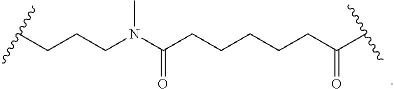 [SMILES: *CCCN(C)C(=O)CCCCCC(*)=O]

In some embodiments, L is

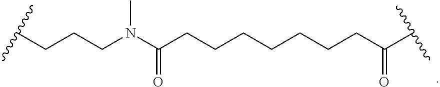 [SMILES: *CCCN(C)C(=O)CCCCCCCC(*)=O]

In some embodiments, L is

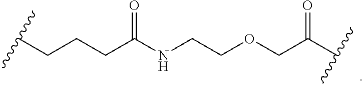 [SMILES: *CCCC(=O)NCCOCC(*)=O]

In some embodiments, L is

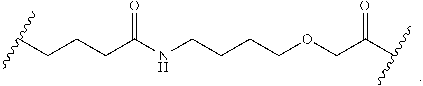 [SMILES: *CCCC(=O)NCCCCOCC(*)=O]

In some embodiments, L is

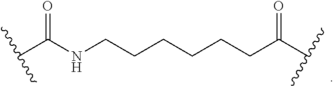 [SMILES: *C(=O)CCCCCCNC(*)=O]

In some embodiments, L is

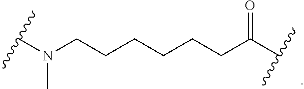 [SMILES: *C(=O)CCCCCCN(*)C]

In some embodiments, L is

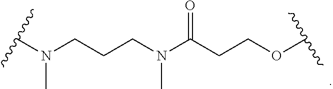 [SMILES: *OCCC(=O)N(C)CCCN(*)C]

In some embodiments, L is

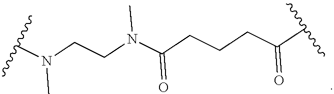 [SMILES: *C(=O)CCCC(=O)N(C)CCN(*)C]

In some embodiments, L is

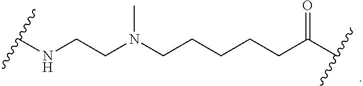 [SMILES: *NCCN(C)CCCCCC(*)=O]

In some embodiments, L is

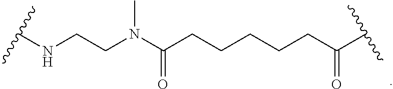 [SMILES: *NCCN(C)C(=O)CCCCCC(*)=O]

In some embodiments, L is

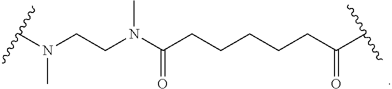 [SMILES: *C(=O)CCCCCC(=O)N(C)CCN(*)C]

In some embodiments, L is

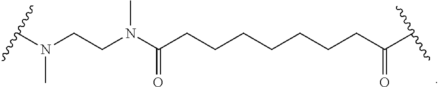 [SMILES: *C(=O)CCCCCCCC(=O)N(C)CCN(*)C]

In some embodiments, L is

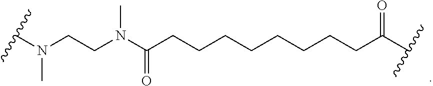 [SMILES: *C(=O)CCCCCCCCC(=O)N(C)CCN(*)C]

In some embodiments, L is

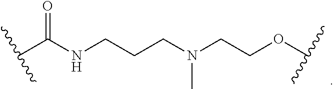 [SMILES: *OCCN(C)CCCNC(*)=O]

In some embodiments, L is

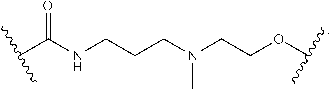 [SMILES: *OCCN(C)CCCNC(*)=O]

In some embodiments, L is

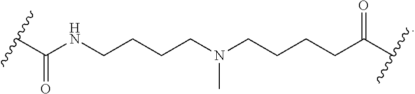 [SMILES: *C(=O)CCCCN(C)CCCCNC(*)=O]

In some embodiments, L is

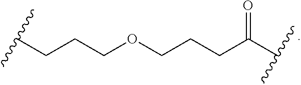 [SMILES: *CCCOCCCC(*)=O]

In some embodiments, L is

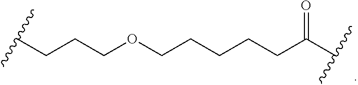 [SMILES: *CCCOCCCCCC(*)=O]

In some embodiments, L is

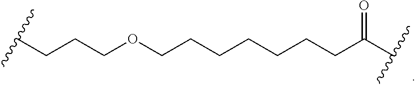 [SMILES: *CCCOCCCCCCCC(*)=O]

In some embodiments, L is —O—. In some embodiments, L is

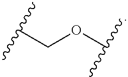 [SMILES: *CO*]

In some embodiments, L is

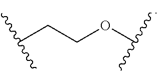 [SMILES: *CCO*]

In some embodiments, L is

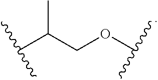 [SMILES: *OCC(*)C]

In some embodiments, L is

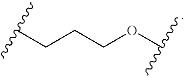 [SMILES: *CCCO*]

In some embodiments, L is

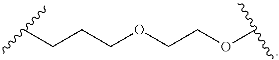 [SMILES: *CCCOCCO*]

In some embodiments, L is

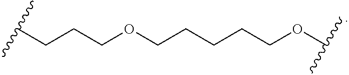 [SMILES: *CCCOCCCCCO*]

In some embodiments, L is

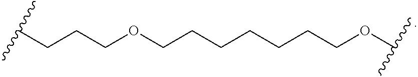 [SMILES: *CCCOCCCCCCCO*]

In some embodiments, L is

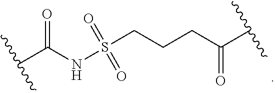 [SMILES: *C(=O)CCCS(=O)(=O)NC(*)=O]

In some embodiments, L is

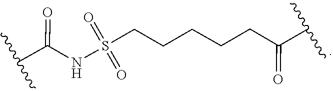 [SMILES: *C(=O)CCCCCS(=O)(=O)NC(*)=O]

In some embodiments, L is

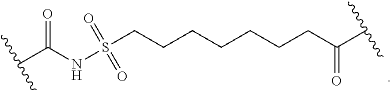 [SMILES: *C(=O)CCCCCCCS(=O)(=O)NC(*)=O]

In some embodiments, L is

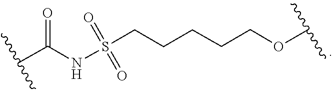 [SMILES: *OCCCCCS(=O)(=O)NC(*)=O]

In some embodiments, L is

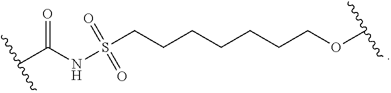 [SMILES: *OCCCCCCCS(=O)(=O)NC(*)=O]

In some embodiments, L is

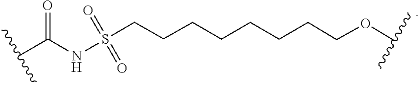 [SMILES: *OCCCCCCCCS(=O)(=O)NC(*)=O]

In some embodiments, L is

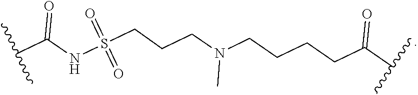 [SMILES: *C(=O)CCCCN(C)CCCS(=O)(=O)NC(*)=O]

In some embodiments, L is

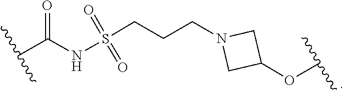 [SMILES: *OC1CN(CCCS(=O)(=O)NC(*)=O)C1]

In some embodiments, L is

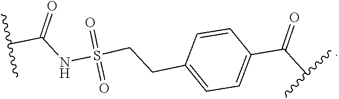 [SMILES: *C(=O)NS(=O)(=O)CCc1ccc(C(*)=O)cc1]

In some embodiments, L is

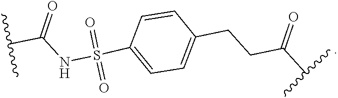 [SMILES: *C(=O)CCc1ccc(S(=O)(=O)NC(*)=O)cc1]

In some embodiments, L is

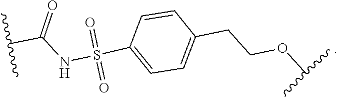 [SMILES: *OCCc1ccc(S(=O)(=O)NC(*)=O)cc1]

In some embodiments, L is

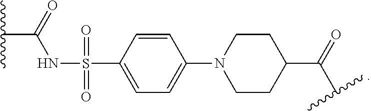 [SMILES: *C(=O)NS(=O)(=O)c1ccc(N2CCC(C(*)=O)CC2)cc1]

In some embodiments, L is

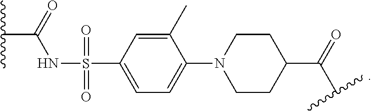 [SMILES: *C(=O)NS(=O)(=O)c1ccc(N2CCC(C(*)=O)CC2)c(C)c1]

In some embodiments, L is

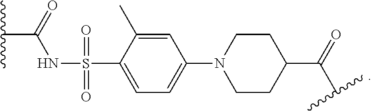 [SMILES: *C(=O)NS(=O)(=O)c1ccc(N2CCC(C(*)=O)CC2)cc1C]

In some embodiments, L is

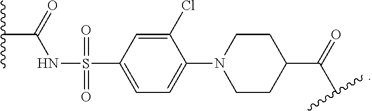 [SMILES: *C(=O)NS(=O)(=O)c1ccc(N2CCC(C(*)=O)CC2)c(Cl)c1]

In some embodiments, L is

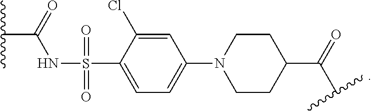 [SMILES: *C(=O)NS(=O)(=O)c1ccc(N2CCC(C(*)=O)CC2)cc1Cl]

In some embodiments, L is

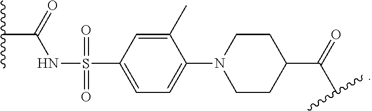 [SMILES: *C(=O)NS(=O)(=O)c1ccc(N2CCC(C(*)=O)CC2)c(C)c1]

In some embodiments, L is

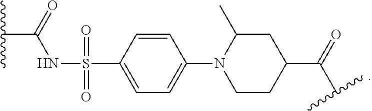 [SMILES: *C(=O)NS(=O)(=O)c1ccc(N2CCC(C(*)=O)CC2C)cc1]

In some embodiments, L is

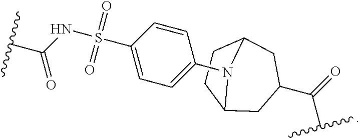 [SMILES: *C(=O)NS(=O)(=O)c1ccc(N2C3CCC2CC(C(*)=O)C3)cc1]

In some embodiments, L is

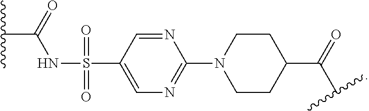 [SMILES: *C(=O)NS(=O)(=O)c1cnc(N2CCC(C(*)=O)CC2)nc1]

In some embodiments, L is

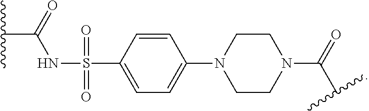 [SMILES: *C(=O)NS(=O)(=O)c1ccc(N2CCN(C(*)=O)CC2)cc1]

In some embodiments, L is

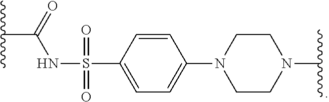 [SMILES: *C(=O)NS(=O)(=O)c1ccc(N2CCN(*)CC2)cc1]

In some embodiments, L is

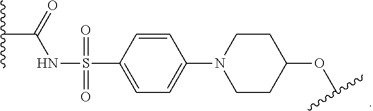 [SMILES: *OC1CCN(c2ccc(S(=O)(=O)NC(*)=O)cc2)CC1]

In some embodiments, L is

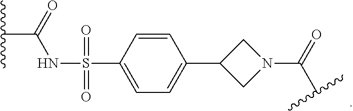 [SMILES: *C(=O)NS(=O)(=O)c1ccc(C2CN(C(*)=O)C2)cc1]

In some embodiments, L is

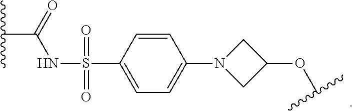 [SMILES: *OC1CN(c2ccc(S(=O)(=O)NC(*)=O)cc2)C1]

In some embodiments, L is

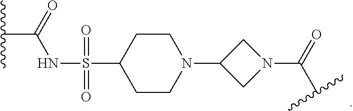 [SMILES: *C(=O)NS(=O)(=O)C1CCN(C2CN(C(*)=O)C2)CC1]

In some embodiments, L is

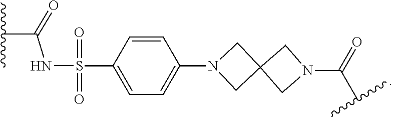 [SMILES: *C(=O)NS(=O)(=O)c1ccc(N2CC3(CN(C(*)=O)C3)C2)cc1]

In some embodiments, L is

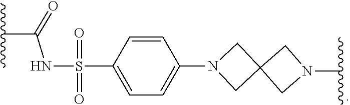 [SMILES: *C(=O)NS(=O)(=O)c1ccc(N2CC3(CN(*)C3)C2)cc1]

In some embodiments, L is

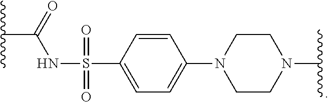 [SMILES: *C(=O)NS(=O)(=O)c1ccc(N2CCN(*)CC2)cc1]

In some embodiments, L is

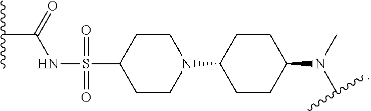 [SMILES: *C(=O)NS(=O)(=O)C1CCN([C@H]2CC[C@H](N(*)C)CC2)CC1]

In some embodiments, L is

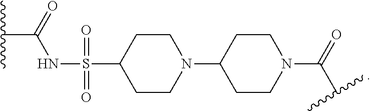 [SMILES: *C(=O)NS(=O)(=O)C1CCN(C2CCN(C(*)=O)CC2)CC1]

In some embodiments, L is

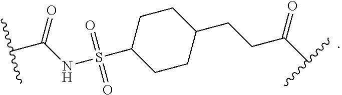 [SMILES: *C(=O)CCC1CCC(S(=O)(=O)NC(*)=O)CC1]

In some embodiments, L is

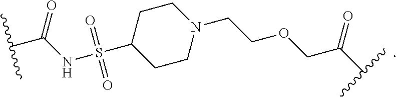 [SMILES: *C(=O)COCCN1CCC(S(=O)(=O)NC(*)=O)CC1]

In some embodiments, L is

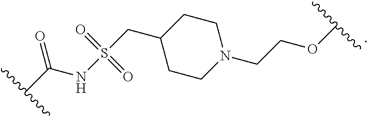 [SMILES: *OCCN1CCC(CS(=O)(=O)NC(*)=O)CC1]

In some embodiments, L is

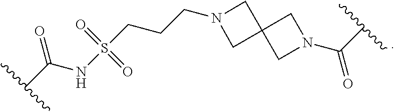 [SMILES: *C(=O)NS(=O)(=O)CCCN1CC2(C1)CN(C(*)=O)C2]

In some embodiments, L is

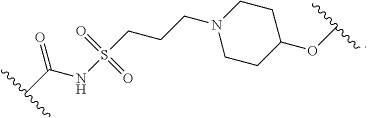 [SMILES: *OC1CCN(CCCS(=O)(=O)NC(*)=O)CC1]

In some embodiments, Lis

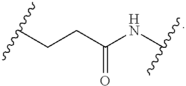 [SMILES: *CCC(=O)N*]

In some embodiments, L is

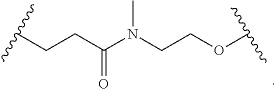 [SMILES: *CCC(=O)N(C)CCO*]

In some embodiments, L is

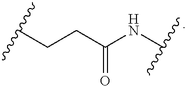 [SMILES: *CCC(=O)N*]

In some embodiments, L is

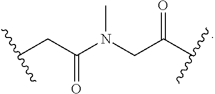 [SMILES: *CC(=O)N(C)CC(*)=O]

In some embodiments, L is

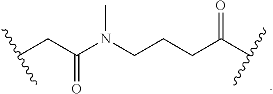 [SMILES: *CC(=O)N(C)CCCC(*)=O]

In some embodiments, L is

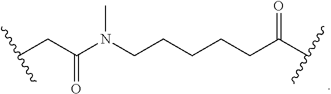 [SMILES: *CC(=O)N(C)CCCCCC(*)=O]

In some embodiments, L is

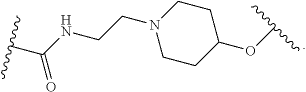 [SMILES: *OC1CCN(CCNC(*)=O)CC1]

In some embodiments, L is

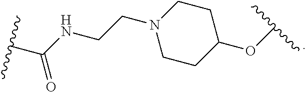 [SMILES: *OC1CCN(CCNC(*)=O)CC1]

In some embodiments, L is

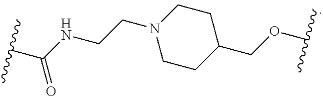 [SMILES: *OCC1CCN(CCNC(*)=O)CC1]

In some embodiments, L is

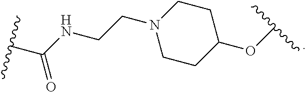 [SMILES: *OC1CCN(CCNC(*)=O)CC1]

In some embodiments, L is

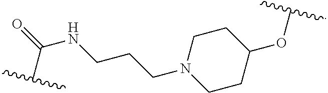 [SMILES: *OC1CCN(CCCNC(*)=O)CC1]

In some embodiments, L is

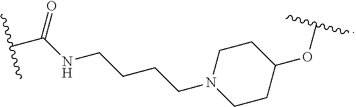 [SMILES: *OC1CCN(CCCCNC(*)=O)CC1]

In some embodiments, L is

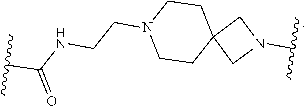 [SMILES: *C(=O)NCCN1CCC2(CC1)CN(*)C2]

In some embodiments, L is

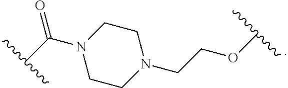 [SMILES: *OCCN1CCN(C(*)=O)CC1]

In some embodiments, L is

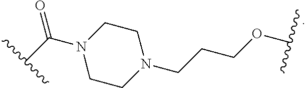 [SMILES: *OCCCN1CCN(C(*)=O)CC1]

In some embodiments, L is

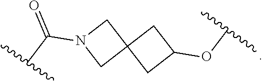 [SMILES: *OC1CC2(C1)CN(C(*)=O)C2]

In some embodiments, L is

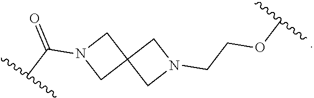 [SMILES: *OCCN1CC2(C1)CN(C(*)=O)C2]

In some embodiments, L is

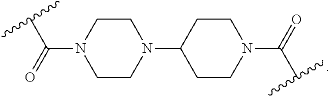 [SMILES: *C(=O)N1CCC(N2CCN(C(*)=O)CC2)CC1]

In some embodiments, L is

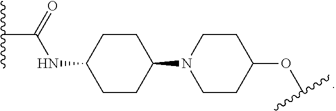 [SMILES: *OC1CCN([C@H]2CC[C@H](NC(*)=O)CC2)CC1]

In some embodiments, L is

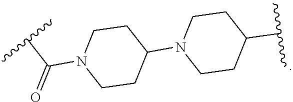 [SMILES: *C(=O)N1CCC(N2CCC(*)CC2)CC1]

In some embodiments, L is

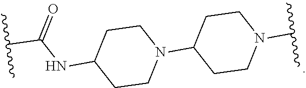 [SMILES: *C(=O)NC1CCN(C2CCN(*)CC2)CC1]

In some embodiments, L is

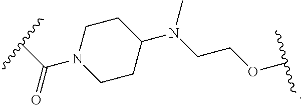 [SMILES: *OCCN(C)C1CCN(C(*)=O)CC1]

In some embodiments, L is

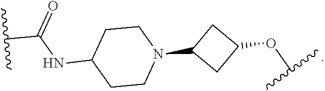

In some embodiments, L is

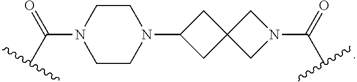 [SMILES: *C(=O)N1CCN(C2CC3(C2)CN(C(*)=O)C3)CC1]

In some embodiments, L is

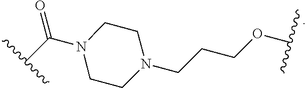 [SMILES: *OCCCN1CCN(C(*)=O)CC1]

In some embodiments, L is

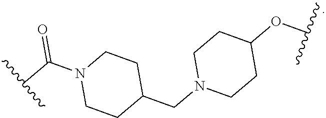 [SMILES: *OC1CCN(CC2CCN(C(*)=O)CC2)CC1]

In some embodiments, L is

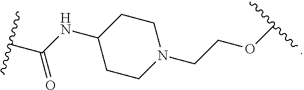 [SMILES: *OCCN1CCC(NC(*)=O)CC1]

In some embodiments, L is

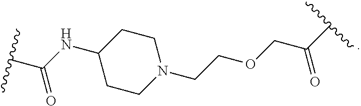 [SMILES: *C(=O)COCCN1CCC(NC(*)=O)CC1]

In some embodiments, L is

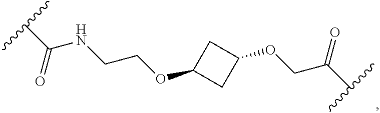 [SMILES: *C(=O)CO[C@H]1C[C@H](OCCNC(*)=O)C1]

In some embodiments, L is

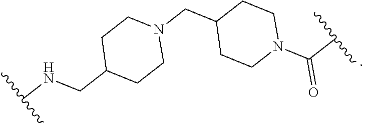 [SMILES: *NCC1CCN(CC2CCN(C(*)=O)CC2)CC1]

In some embodiments, L is

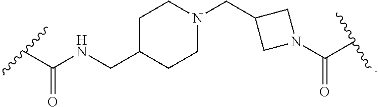 [SMILES: *C(=O)NCC1CCN(CC2CN(C(*)=O)C2)CC1]

In some embodiments, L is

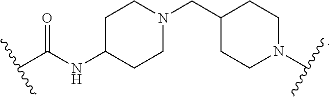 [SMILES: *C(=O)NC1CCN(CC2CCN(*)CC2)CC1]

In some embodiments, L is

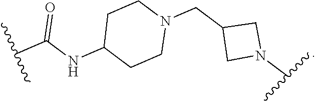 [SMILES: *C(=O)NC1CCN(CC2CN(*)C2)CC1]

In some embodiments, L is

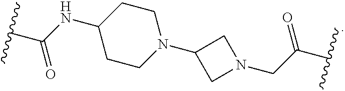 [SMILES: *C(=O)CN1CC(N2CCC(NC(*)=O)CC2)C1]

In some embodiments, L is

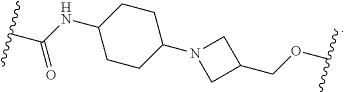 [SMILES: *OCC1CN(C2CCC(NC(*)=O)CC2)C1]

In some embodiments, L is

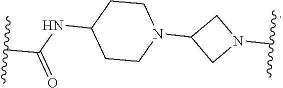 [SMILES: *C(=O)NC1CCN(C2CN(*)C2)CC1]

In some embodiments, L is

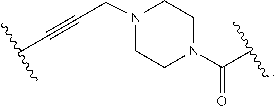 [SMILES: *C#CCN1CCN(C(*)=O)CC1]

In some embodiments, L is

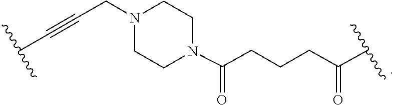 [SMILES: *C#CCN1CCN(C(=O)CCCC(*)=O)CC1]

In some embodiments, L is

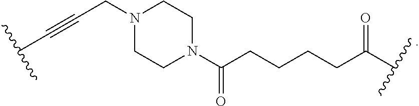 [SMILES: *C#CCN1CCN(C(=O)CCCCC(*)=O)CC1]

In some embodiments, L is

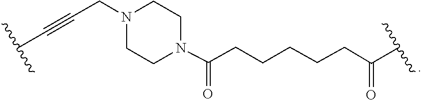 [SMILES: *C#CCN1CCN(C(=O)CCCCCC(*)=O)CC1]

In some embodiments, L is

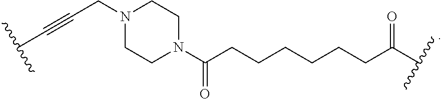 [SMILES: *C#CCN1CCN(C(=O)CCCCCCC(*)=O)CC1]

In some embodiments, L is

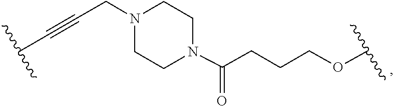 [SMILES: *C#CCN1CCN(C(=O)CCCO*)CC1]

In some embodiments, L is

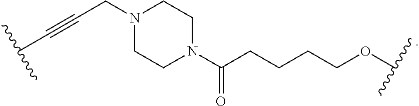 [SMILES: *C#CCN1CCN(C(=O)CCCCO*)CC1]

In some embodiments, L is

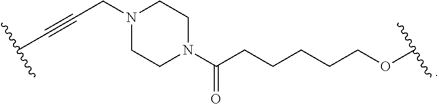 [SMILES: *C#CCN1CCN(C(=O)CCCCCO*)CC1]

In some embodiments, L is

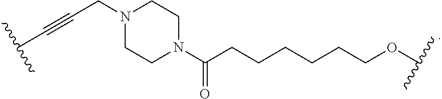 [SMILES: *C#CCN1CCN(C(=O)CCCCCCO*)CC1]

In some embodiments, L is

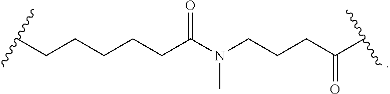 [SMILES: *CCCCCC(=O)N(C)CCCC(*)=O]

In some embodiments, L is

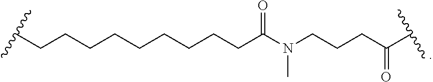 [SMILES: *CCCCCCCCCC(=O)N(C)CCCC(*)=O]

In some embodiments, L is

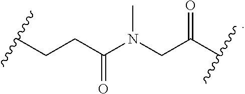 [SMILES: *CCC(=O)N(C)CC(*)=O]

In some embodiments, L is

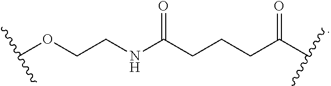 [SMILES: *OCCNC(=O)CCCC(*)=O]

In some embodiments, L is

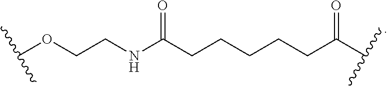 [SMILES: *OCCNC(=O)CCCCCC(*)=O]

In some embodiments, L is

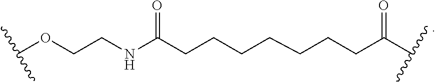 [SMILES: *OCCNC(=O)CCCCCCCC(*)=O]

In some embodiments, L is

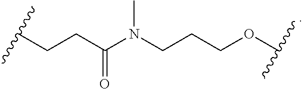 [SMILES: *CCC(=O)N(C)CCCO*]

In some embodiments, L is

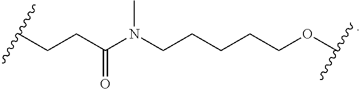 [SMILES: *CCC(=O)N(C)CCCCCO*]

In some embodiments, L is

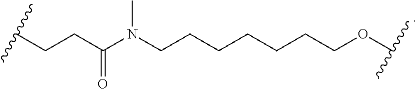 [SMILES: *CCC(=O)N(C)CCCCCCCO*]

In some embodiments, L is

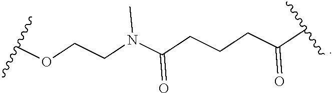 [SMILES: *OCCN(C)C(=O)CCCC(*)=O]

In some embodiments, L is

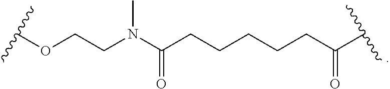 [SMILES: *OCCN(C)C(=O)CCCCCC(*)=O]

In some embodiments, L is

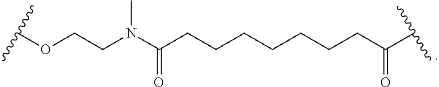 [SMILES: *OCCN(C)C(=O)CCCCCCCC(*)=O]

In some embodiments, L is

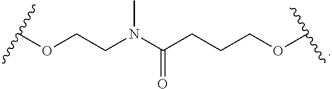 [SMILES: *OCCCC(=O)N(C)CCO*]

In some embodiments, L is

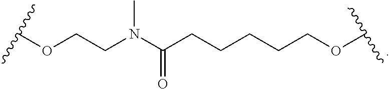 [SMILES: *OCCCCCC(=O)N(C)CCO*]

In some embodiments, L is

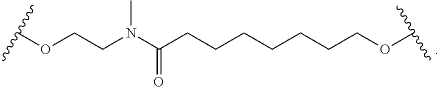 [SMILES: *OCCCCCCCC(=O)N(C)CCO*]

In some embodiments, L is

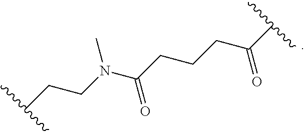 [SMILES: *CCN(C)C(=O)CCCC(*)=O]

In some embodiments, L is

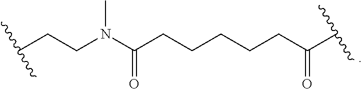 [SMILES: *CCN(C)C(=O)CCCCCC(*)=O]

In some embodiments, L is

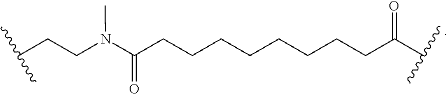 [SMILES: *CCN(C)C(=O)CCCCCCCCC(*)=O]

In some embodiments, L is

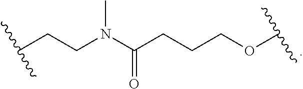 [SMILES: *CCN(C)C(=O)CCCO*]

In some embodiments, L is

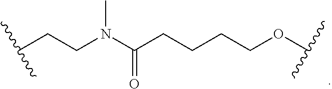 [SMILES: *CCN(C)C(=O)CCCCO*]

In some embodiments, L is

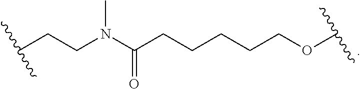 [SMILES: *CCN(C)C(=O)CCCCCO*]

In some embodiments, L is

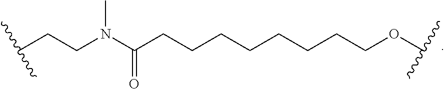 [SMILES: *CCN(C)C(=O)CCCCCCCCO*]

In some embodiments, L is

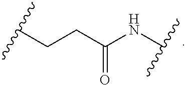 [SMILES: *CCC(=O)N*]

In some embodiments, L is

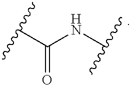 [SMILES: *NC(*)=O]

In some embodiments, L is

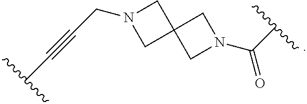 [SMILES: *C#CCN1CC2(C1)CN(C(*)=O)C2]

In some embodiments, L is

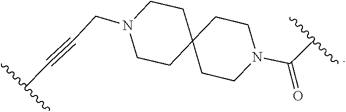 [SMILES: *C#CCN1CCC2(CC1)CCN(C(*)=O)CC2]

In some embodiments, L is

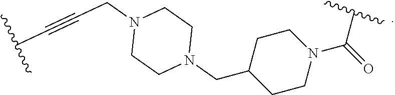 [SMILES: *C#CCN1CCN(CC2CCN(C(*)=O)CC2)CC1]

In some embodiments, L is

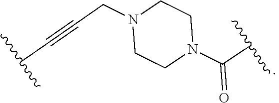 [SMILES: *C#CCN1CCN(C(*)=O)CC1]

In some embodiments, L is

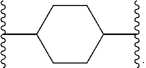 [SMILES: *C1CCC(*)CC1]

In some embodiments, L is

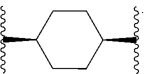 [SMILES: *[C@H]1CC[C@@H](*)CC1]

In some embodiments, L is

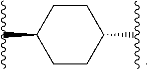 [SMILES: *[C@H]1CC[C@H](*)CC1]

In some embodiments, L is

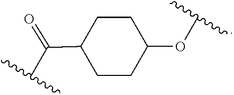 [SMILES: *OC1CCC(C(*)=O)CC1]

In some embodiments, L is

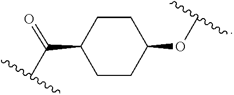 [SMILES: *O[C@H]1CC[C@@H](C(*)=O)CC1]

In some embodiments, L is

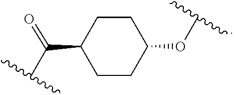 [SMILES: *O[C@H]1CC[C@H](C(*)=O)CC1]

In some embodiments, L is

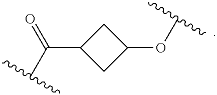 [SMILES: *OC1CC(C(*)=O)C1]

In some embodiments, L is

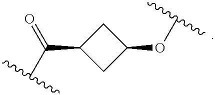 [SMILES: *O[C@H]1C[C@@H](C(*)=O)C1]

In some embodiments, L is

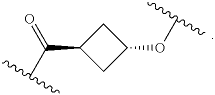 [SMILES: *O[C@H]1C[C@H](C(*)=O)C1]

In some embodiments, L is

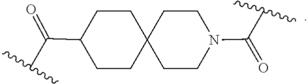 [SMILES: *C(=O)C1CCC2(CC1)CCN(C(*)=O)CC2]

In some embodiments, L is

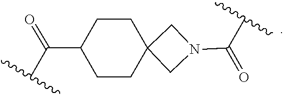 [SMILES: *C(=O)C1CCC2(CC1)CN(C(*)=O)C2]

In some embodiments, L is

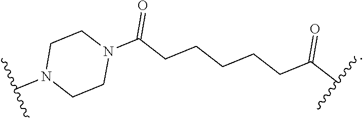 [SMILES: *C(=O)CCCCCC(=O)N1CCN(*)CC1]

In some embodiments, L is

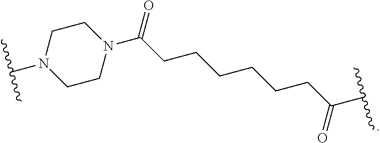 [SMILES: *C(=O)CCCCCCC(=O)N1CCN(*)CC1]

In some embodiments, L is

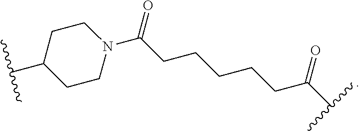 [SMILES: *C(=O)CCCCCC(=O)N1CCC(*)CC1]

In some embodiments, L is

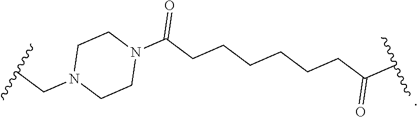 [SMILES: *CN1CCN(C(=O)CCCCCCC(*)=O)CC1]

In some embodiments, L is

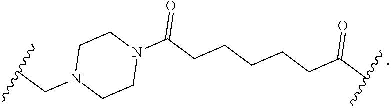 [SMILES: *CN1CCN(C(=O)CCCCCC(*)=O)CC1]

In some embodiments, L is

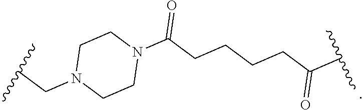 [SMILES: *CN1CCN(C(=O)CCCCC(*)=O)CC1]

In some embodiments, L is

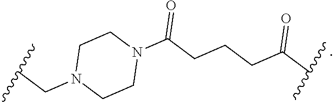 [SMILES: *CN1CCN(C(=O)CCCC(*)=O)CC1]

In some embodiments, L is

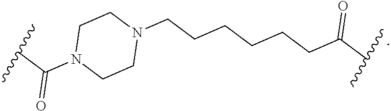 [SMILES: *C(=O)CCCCCCN1CCN(C(*)=O)CC1]

In some embodiments, L is

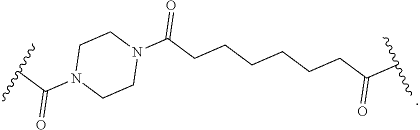 [SMILES: *C(=O)CCCCCCC(=O)N1CCN(C(*)=O)CC1]

In some embodiments, L is

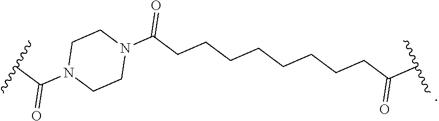 [SMILES: *C(=O)CCCCCCCCC(=O)N1CCN(C(*)=O)CC1]

In some embodiments, L is

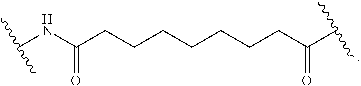 [SMILES: *NC(=O)CCCCCCCC(*)=O]

In some embodiments, L is

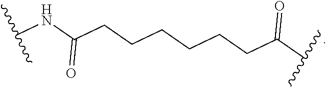 [SMILES: *NC(=O)CCCCCCC(*)=O]

In some embodiments, L is

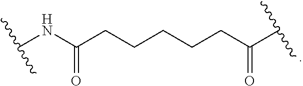 [SMILES: *NC(=O)CCCCCC(*)=O]

In some embodiments, L is

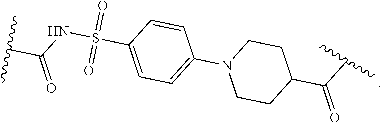 [SMILES: *C(=O)NS(=O)(=O)c1ccc(N2CCC(C(*)=O)CC2)cc1]

In some embodiments, L is

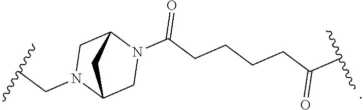 [SMILES: *CN1C[C@H]2C[C@@H]1CN2C(=O)CCCCC(*)=O]

In some embodiments, L is

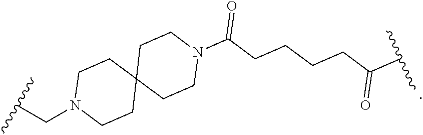 [SMILES: *CN1CCC2(CC1)CCN(C(=O)CCCCC(*)=O)CC2]

In some embodiments, L is

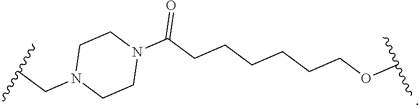 [SMILES: *CN1CCN(C(=O)CCCCCCO*)CC1]

In some embodiments, L is

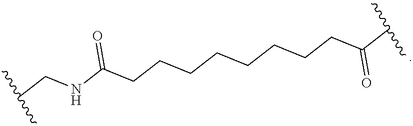 [SMILES: *CNC(=O)CCCCCCCCC(*)=O]

In some embodiments, L is

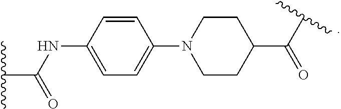 [SMILES: *C(=O)Nc1ccc(N2CCC(C(*)=O)CC2)cc1]

In some embodiments, L is

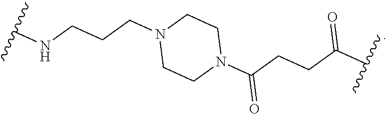 [SMILES: *NCCCN1CCN(C(=O)CCC(*)=O)CC1]

In some embodiments, L is

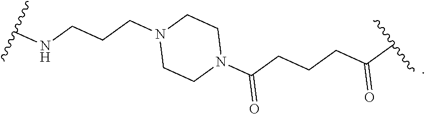 [SMILES: *NCCCN1CCN(C(=O)CCCC(*)=O)CC1]

In some embodiments, L is

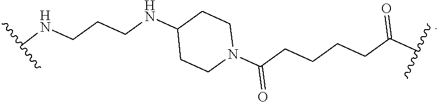 [SMILES: *NCCCNC1CCN(C(=O)CCCCC(*)=O)CC1]

In some embodiments, L is

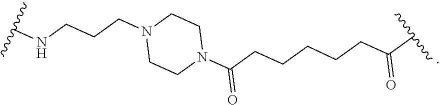 [SMILES: *NCCCN1CCN(C(=O)CCCCCC(*)=O)CC1]

In some embodiments, L is

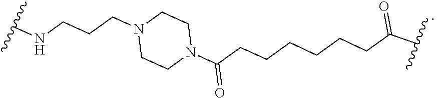 [SMILES: *NCCCN1CCN(C(=O)CCCCCCC(*)=O)CC1]

In some embodiments, L is

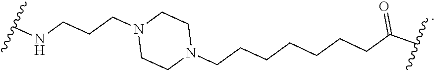 [SMILES: *NCCCN1CCN(CCCCCCCC(*)=O)CC1]

In some embodiments, L is

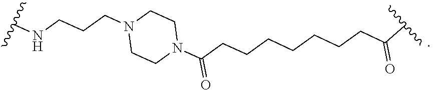 [SMILES: *NCCCN1CCN(C(=O)CCCCCCCC(*)=O)CC1]

In some embodiments, L is

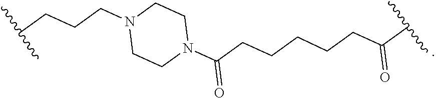 [SMILES: *CCCN1CCN(C(=O)CCCCCC(*)=O)CC1]

In some embodiments, L is

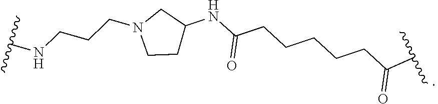 [SMILES: *NCCCN1CCC(NC(=O)CCCCCC(*)=O)C1]

In some embodiments, L is

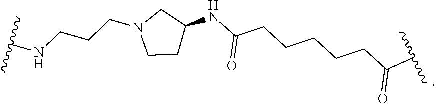 [SMILES: *NCCCN1CC[C@H](NC(=O)CCCCCC(*)=O)C1]

In some embodiments, L is

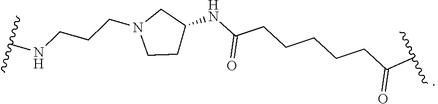 [SMILES: *NCCCN1CC[C@@H](NC(=O)CCCCCC(*)=O)C1]

In some embodiments, L is

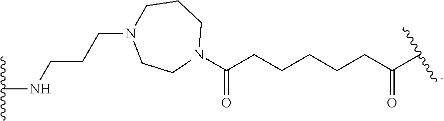 [SMILES: *NCCCN1CCCN(C(=O)CCCCCC(*)=O)CC1]

In some embodiments, L is

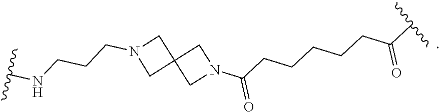 [SMILES: *NCCCN1CC2(C1)CN(C(=O)CCCCCC(*)=O)C2]

In some embodiments, L is

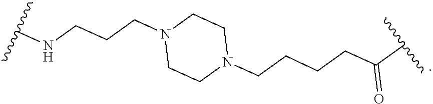 [SMILES: *NCCCN1CCN(CCCCC(*)=O)CC1]

In some embodiments, L is

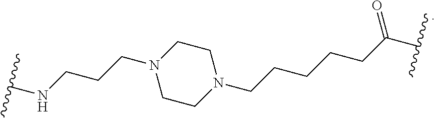 [SMILES: *NCCCN1CCN(CCCCCC(*)=O)CC1]

In some embodiments, L is

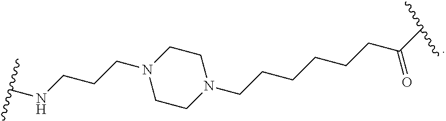 [SMILES: *NCCCN1CCN(CCCCCCC(*)=O)CC1]

In some embodiments, L is

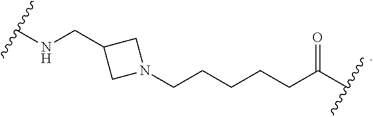 [SMILES: *NCC1CN(CCCCCC(*)=O)C1]

In some embodiments, L is

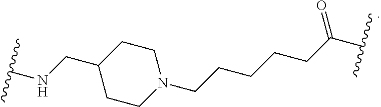 [SMILES: *NCC1CCN(CCCCCC(*)=O)CC1]

In some embodiments, L is

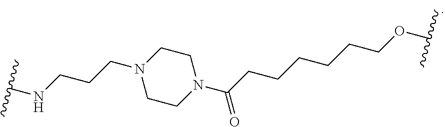 [SMILES: *NCCCN1CCN(C(=O)CCCCCCO*)CC1]

In some embodiments, L is

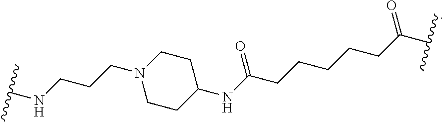 [SMILES: *NCCCN1CCC(NC(=O)CCCCCC(*)=O)CC1]

In some embodiments, L is

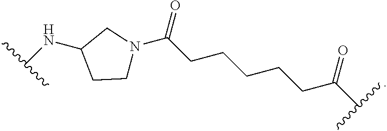 [SMILES: *NC1CCN(C(=O)CCCCCC(*)=O)C1]

In some embodiments, L is

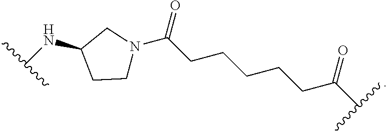 [SMILES: *N[C@@H]1CCN(C(=O)CCCCCC(*)=O)C1]

In some embodiments, L is

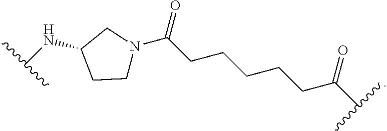 [SMILES: *N[C@H]1CCN(C(=O)CCCCCC(*)=O)C1]

In some embodiments, L is

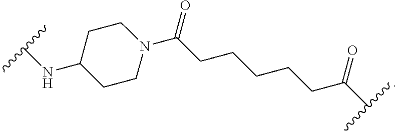 [SMILES: *NC1CCN(C(=O)CCCCCC(*)=O)CC1]

In some embodiments, L is

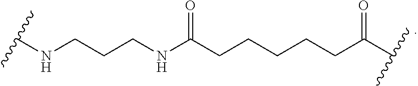 [SMILES: *NCCCNC(=O)CCCCCC(*)=O]

In some embodiments, L is

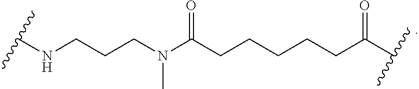 [SMILES: *NCCCN(C)C(=O)CCCCCC(*)=O]

In some embodiments, L is

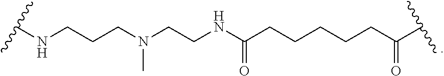 [SMILES: *NCCCN(C)CCNC(=O)CCCCCC(*)=O]

In some embodiments, L is

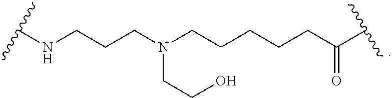 [SMILES: *NCCCN(CCO)CCCCCC(*)=O]

In some embodiments, L is

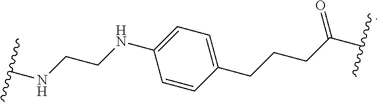 [SMILES: *NCCNc1ccc(CCCC(*)=O)cc1]

In some embodiments, L is

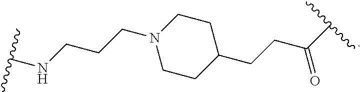 [SMILES: *NCCCN1CCC(CCC(*)=O)CC1]

In some embodiments, L is

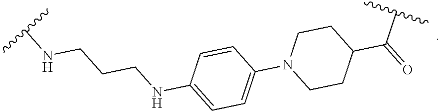 [SMILES: *NCCCNc1ccc(N2CCC(C(*)=O)CC2)cc1]

In some embodiments, L is

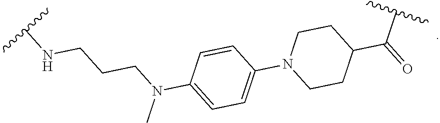 [SMILES: *NCCCN(C)c1ccc(N2CCC(C(*)=O)CC2)cc1]

In some embodiments, L is

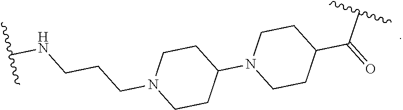 [SMILES: *NCCCN1CCC(N2CCC(C(*)=O)CC2)CC1]

In some embodiments, L is

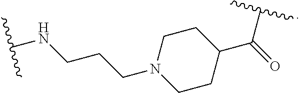 [SMILES: *NCCCN1CCC(C(*)=O)CC1]

In some embodiments, L is

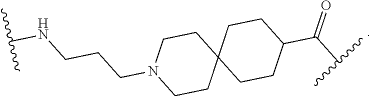 [SMILES: *NCCCN1CCC2(CCC(C(*)=O)CC2)CC1]

In some embodiments, L is

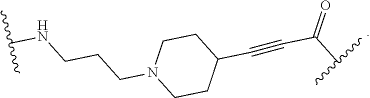 [SMILES: *NCCCN1CCC(C#CC(*)=O)CC1]

In some embodiments, L is

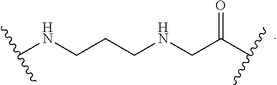 [SMILES: *NCCCNCC(*)=O]

In some embodiments, L is selected from those depicted in Table B, below.

In some embodiments, L is selected from those depicted in Table 1, below.

Without limitation, the point of attachment of L to BBM and DIM can be, for example when L is

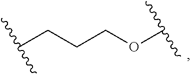 [SMILES: *CCCO*]

either

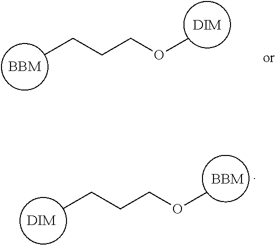

In some embodiments, a provided compound or pharmaceutically acceptable salt thereof, is selected from those wherein BBM is

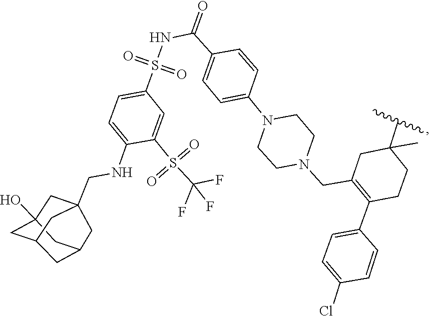 [SMILES: *C1(C)CCC(c2ccc(Cl)cc2)=C(CN2CCN(c3ccc(C(=O)NS(=O)(=O)c4ccc(NCC56CC7CC(CC(O)(C7)C5)C6)c(S(=O)(=O)C(F)(F)F)c4)cc3)CC2)C1]

LBM is selected from any of those in Table A below, and L is selected from any of those in Table B below.

In some embodiments, a provided compound or pharmaceutically acceptable salt thereof, is selected from those wherein BBM is

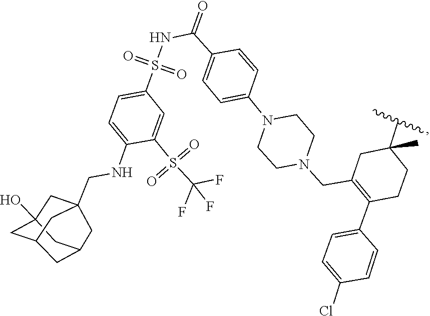 [SMILES: *[C@]1(C)CCC(c2ccc(Cl)cc2)=C(CN2CCN(c3ccc(C(=O)NS(=O)(=O)c4ccc(NCC56CC7CC(CC(O)(C7)C5)C6)c(S(=O)(=O)C(F)(F)F)c4)cc3)CC2)C1]

LBM is selected from any of those in Table A below, and L is selected from any of those in Table B below.

In some embodiments, a provided compound or pharmaceutically acceptable salt thereof, is selected from those wherein BBM is

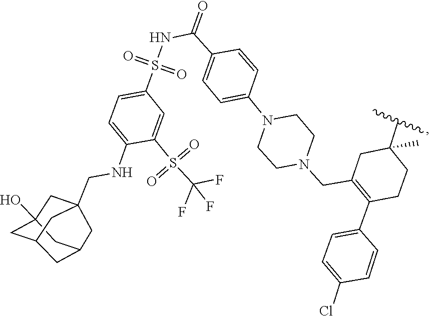 [SMILES: *[C@@]1(C)CCC(c2ccc(Cl)cc2)=C(CN2CCN(c3ccc(C(=O)NS(=O)(=O)c4ccc(NCC56CC7CC(CC(O)(C7)C5)C6)c(S(=O)(=O)C(F)(F)F)c4)cc3)CC2)C1]

LBM is selected from any of those in Table A below, and L is selected from any of those in Table B below.

In some embodiments, a provided compound or pharmaceutically acceptable salt thereof, is selected from those wherein BBM is

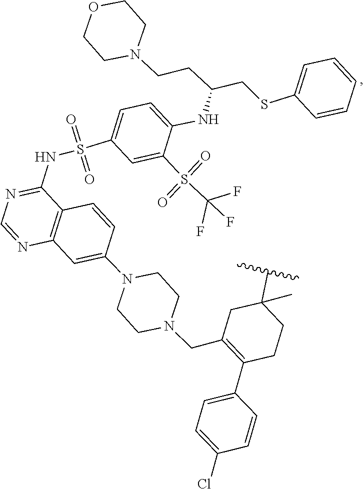 [SMILES: *C1(C)CCC(c2ccc(Cl)cc2)=C(CN2CCN(c3ccc4c(NS(=O)(=O)c5ccc(N[C@H](CCN6CCOCC6)CSc6ccccc6)c(S(=O)(=O)C(F)(F)F)c5)ncnc4c3)CC2)C1]

LBM is selected from any of those in Table A below, and L is selected from any of those in Table B below.

In some embodiments, a provided compound or pharmaceutically acceptable salt thereof, is selected from those wherein BBM is

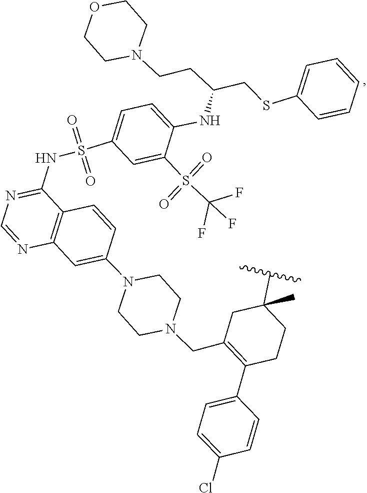 [SMILES: *[C@]1(C)CCC(c2ccc(Cl)cc2)=C(CN2CCN(c3ccc4c(NS(=O)(=O)c5ccc(N[C@H](CCN6CCOCC6)CSc6ccccc6)c(S(=O)(=O)C(F)(F)F)c5)ncnc4c3)CC2)C1]

LBM is selected from any of those in Table A below, and L is selected from any of those in Table B below.

In some embodiments, a provided compound or pharmaceutically acceptable salt thereof, is selected from those wherein BBM is

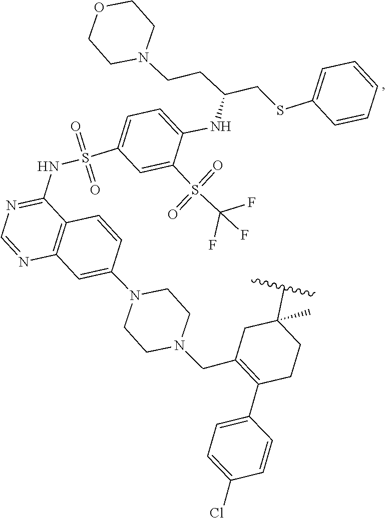 [SMILES: *[C@@]1(C)CCC(c2ccc(Cl)cc2)=C(CN2CCN(c3ccc4c(NS(=O)(=O)c5ccc(N[C@H](CCN6CCOCC6)CSc6ccccc6)c(S(=O)(=O)C(F)(F)F)c5)ncnc4c3)CC2)C1]

LBM is selected from any of those in Table A below, and L is selected from any of those in Table B below.

In some embodiments, a provided compound or pharmaceutically acceptable salt thereof, is selected from those wherein BBM is

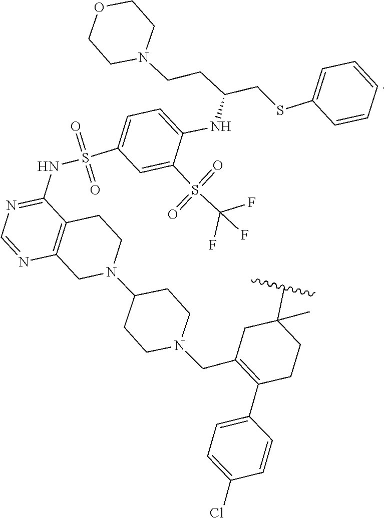 [SMILES: *C1(C)CCC(c2ccc(Cl)cc2)=C(CN2CCC(N3CCc4c(ncnc4NS(=O)(=O)c4ccc(N[C@H](CCN5CCOCC5)CSc5ccccc5)c(S(=O)(=O)C(F)(F)F)c4)C3)CC2)C1]

LBM is selected from any of those in Table A below, and L is selected from any of those in Table B below.

In some embodiments, a provided compound or pharmaceutically acceptable salt thereof, is selected from those wherein BBM is

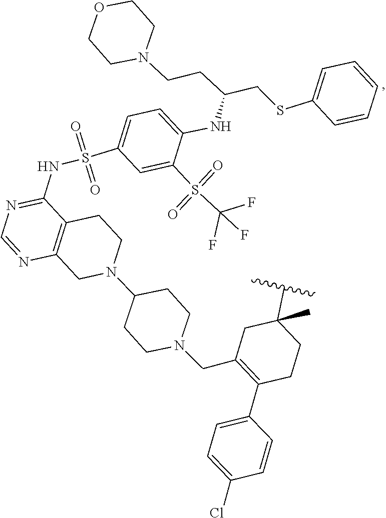 [SMILES: *[C@]1(C)CCC(c2ccc(Cl)cc2)=C(CN2CCC(N3CCc4c(ncnc4NS(=O)(=O)c4ccc(N[C@H](CCN5CCOCC5)CSc5ccccc5)c(S(=O)(=O)C(F)(F)F)c4)C3)CC2)C1]

LBM is selected from any of those in Table A below, and L is selected from any of those in Table B below.

In some embodiments, a provided compound or pharmaceutically acceptable salt thereof, is selected from those wherein BBM is

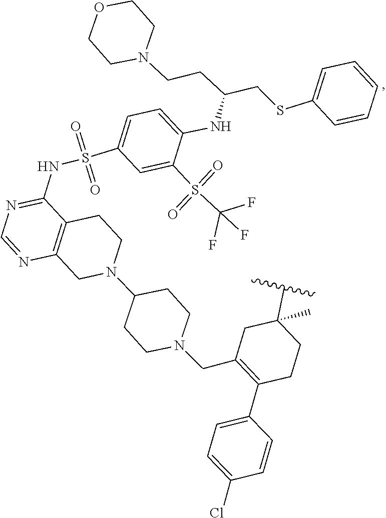 [SMILES: *[C@@]1(C)CCC(c2ccc(Cl)cc2)=C(CN2CCC(N3CCc4c(ncnc4NS(=O)(=O)c4ccc(N[C@H](CCN5CCOCC5)CSc5ccccc5)c(S(=O)(=O)C(F)(F)F)c4)C3)CC2)C1]

LBM is selected from any of those in Table A below, and L is selected from any of those in Table B below.

In some embodiments, a provided compound or pharmaceutically acceptable salt thereof, is selected from those wherein BBM is

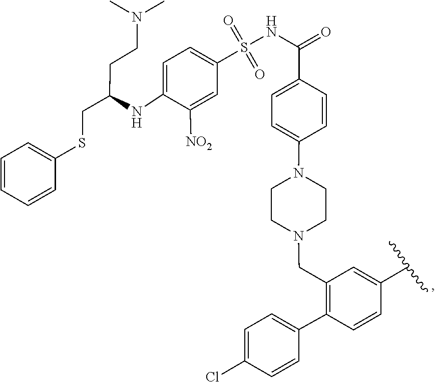 [SMILES: *c1ccc(-c2ccc(Cl)cc2)c(CN2CCN(c3ccc(C(=O)NS(=O)(=O)c4ccc(N[C@H](CCN(C)C)CSc5ccccc5)c([N+](=O)[O-])c4)cc3)CC2)c1]

LBM is selected from any of those in Table A below, and L is selected from any of those in Table B below.

In some embodiments, a provided compound or pharmaceutically acceptable salt thereof, is selected from those wherein BBM is

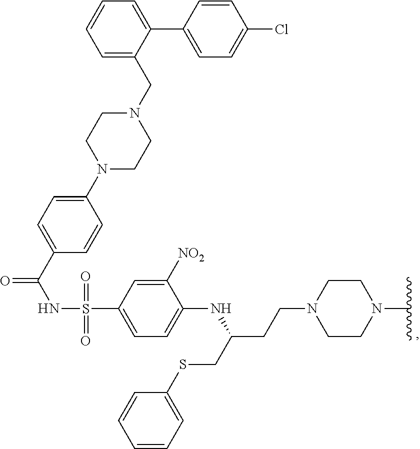 [SMILES: *N1CCN(CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])CC1]

LBM is selected from any of those in Table A below, and L is selected from any of those in Table B below.

In some embodiments, a provided compound or pharmaceutically acceptable salt thereof, is selected from those wherein BBM is

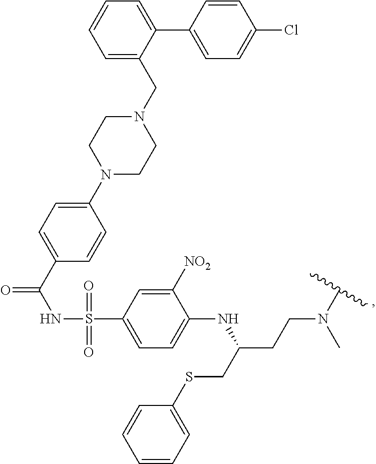 [SMILES: *N(C)CC[C@H](CSc1ccccc1)Nc1ccc(S(=O)(=O)NC(=O)c2ccc(N3CCN(Cc4ccccc4-c4ccc(Cl)cc4)CC3)cc2)cc1[N+](=O)[O-]]

LBM is selected from any of those in Table A below, and L is selected from any of those in Table B below.

In some embodiments, a provided compound or pharmaceutically acceptable salt thereof, is selected from those wherein BBM is

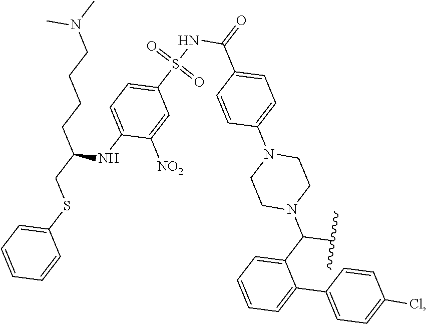 [SMILES: *C(c1ccccc1-c1ccc(Cl)cc1)N1CCN(c2ccc(C(=O)NS(=O)(=O)c3ccc(N[C@H](CCCCN(C)C)CSc4ccccc4)c([N+](=O)[O-])c3)cc2)CC1]

LBM is selected from any of those in Table A below, and L is selected from any of those in Table B below.

In some embodiments, a provided compound or pharmaceutically acceptable salt thereof, is selected from those wherein BBM is

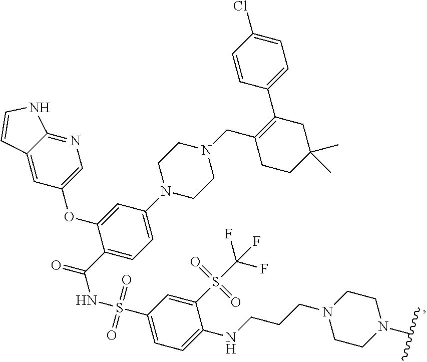 [SMILES: *N1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

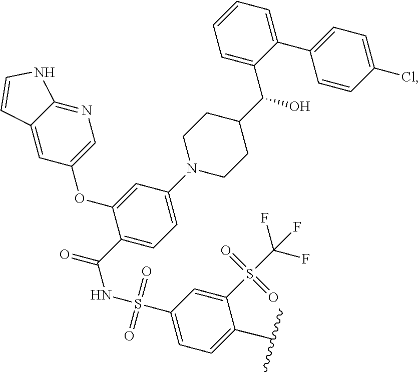 [SMILES: *c1ccc(S(=O)(=O)NC(=O)c2ccc(N3CCC([C@@H](O)c4ccccc4-c4ccc(Cl)cc4)CC3)cc2Oc2cnc3[nH]ccc3c2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

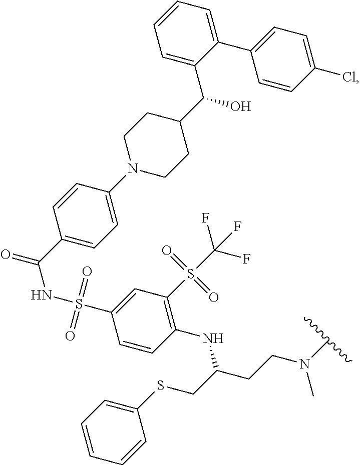

LBM is selected from any of those in Table A below, and L is selected from any of those in Table B below.

In some embodiments, a provided compound or pharmaceutically acceptable salt thereof, is selected from those wherein BBM is

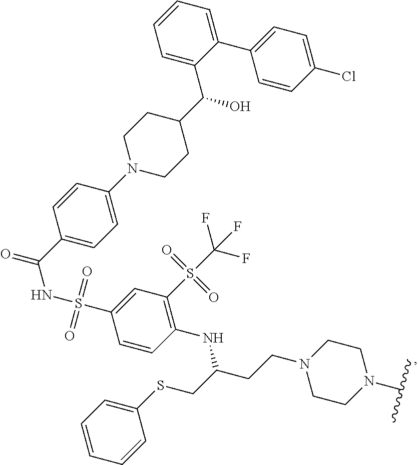

LBM is selected from any of those in Table A below, and L is selected from any of those in Table B below.

In some embodiments, a provided compound or pharmaceutically acceptable salt thereof, is selected from those wherein BBM is

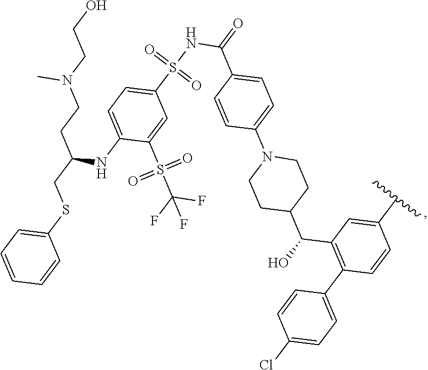 [SMILES: *c1ccc(-c2ccc(Cl)cc2)c([C@H](O)C2CCN(c3ccc(C(=O)NS(=O)(=O)c4ccc(N[C@H](CCN(C)CCO)CSc5ccccc5)c(S(=O)(=O)C(F)(F)F)c4)cc3)CC2)c1]

LBM is selected from any of those in Table A below, and L is selected from any of those in Table B below.

In some embodiments, a provided compound or pharmaceutically acceptable salt thereof, is selected from those wherein BBM is

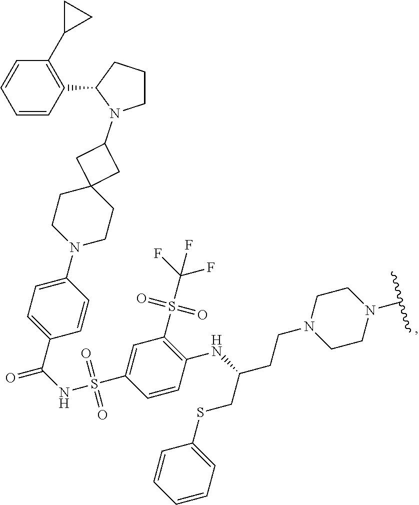 [SMILES: *N1CCN(CC[C@H](CSc2ccccc2)Nc2ccc(S(=O)(=O)NC(=O)c3ccc(N4CCC5(CC4)CC(N4CCC[C@H]4c4ccccc4C4CC4)C5)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

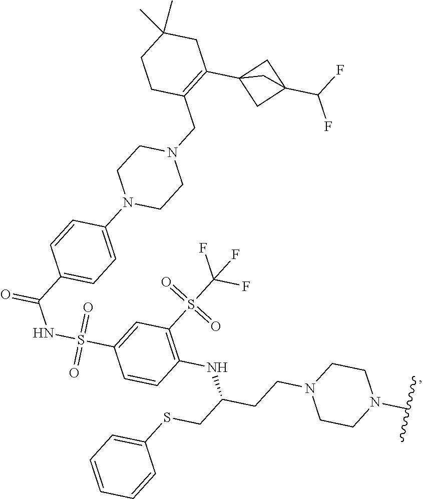 [SMILES: *N1CCN(CC[C@H](CSc2ccccc2)Nc2ccc(S(=O)(=O)NC(=O)c3ccc(N4CCN(CC5=C(C67CC(C(F)F)(C6)C7)CC(C)(C)CC5)CC4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

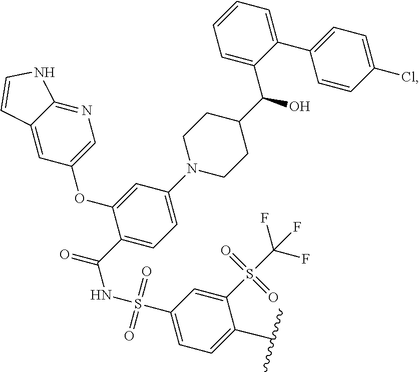 [SMILES: *c1ccc(S(=O)(=O)NC(=O)c2ccc(N3CCC([C@H](O)c4ccccc4-c4ccc(Cl)cc4)CC3)cc2Oc2cnc3[nH]ccc3c2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

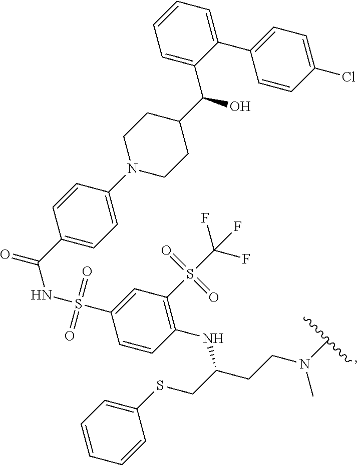 [SMILES: *N(C)CC[C@H](CSc1ccccc1)Nc1ccc(S(=O)(=O)NC(=O)c2ccc(N3CCC([C@H](O)c4ccccc4-c4ccc(Cl)cc4)CC3)cc2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

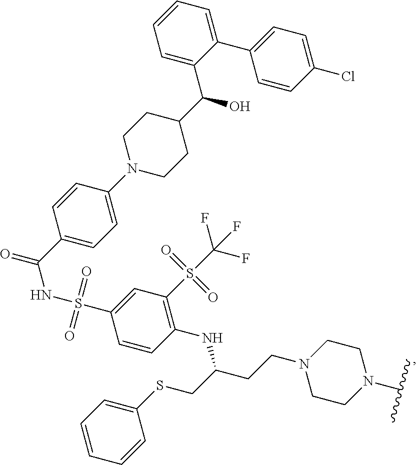 [SMILES: *N1CCN(CC[C@H](CSc2ccccc2)Nc2ccc(S(=O)(=O)NC(=O)c3ccc(N4CCC([C@H](O)c5ccccc5-c5ccc(Cl)cc5)CC4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

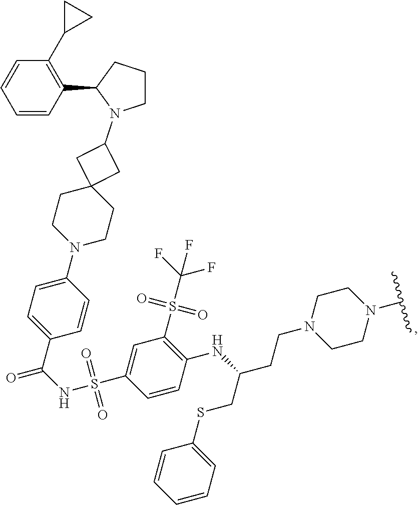 [SMILES: *N1CCN(CC[C@H](CSc2ccccc2)Nc2ccc(S(=O)(=O)NC(=O)c3ccc(N4CCC5(CC4)CC(N4CCC[C@@H]4c4ccccc4C4CC4)C5)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

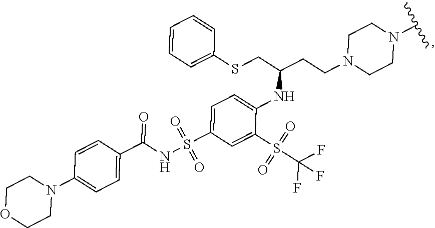 [SMILES: *N1CCN(CC[C@H](CSc2ccccc2)Nc2ccc(S(=O)(=O)NC(=O)c3ccc(N4CCOCC4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

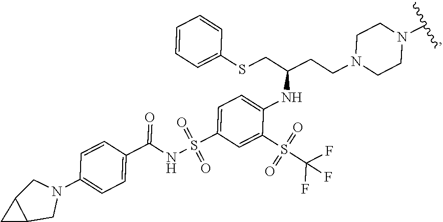 [SMILES: *N1CCN(CC[C@H](CSc2ccccc2)Nc2ccc(S(=O)(=O)NC(=O)c3ccc(N4CC5CC5C4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

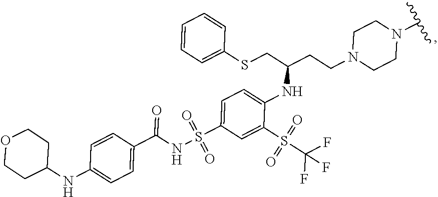 [SMILES: *N1CCN(CC[C@H](CSc2ccccc2)Nc2ccc(S(=O)(=O)NC(=O)c3ccc(NC4CCOCC4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

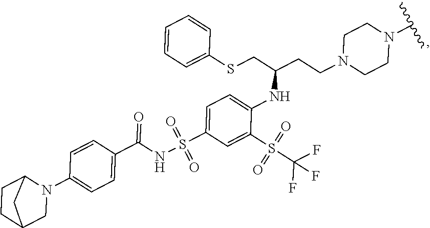 [SMILES: *N1CCN(CC[C@H](CSc2ccccc2)Nc2ccc(S(=O)(=O)NC(=O)c3ccc(N4CC5CCC4C5)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

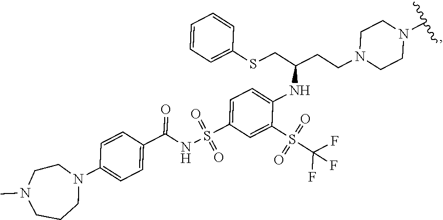 [SMILES: *N1CCN(CC[C@H](CSc2ccccc2)Nc2ccc(S(=O)(=O)NC(=O)c3ccc(N4CCCN(C)CC4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

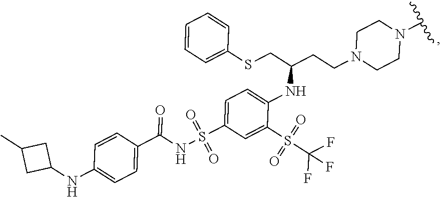 [SMILES: *N1CCN(CC[C@H](CSc2ccccc2)Nc2ccc(S(=O)(=O)NC(=O)c3ccc(NC4CC(C)C4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

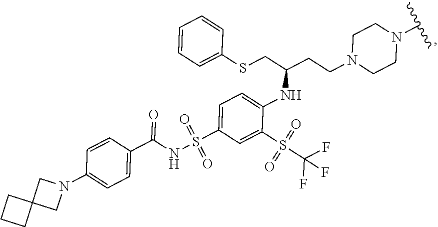 [SMILES: *N1CCN(CC[C@H](CSc2ccccc2)Nc2ccc(S(=O)(=O)NC(=O)c3ccc(N4CC5(CCC5)C4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

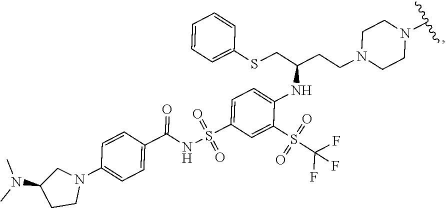 [SMILES: *N1CCN(CC[C@H](CSc2ccccc2)Nc2ccc(S(=O)(=O)NC(=O)c3ccc(N4CC[C@@H](N(C)C)C4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

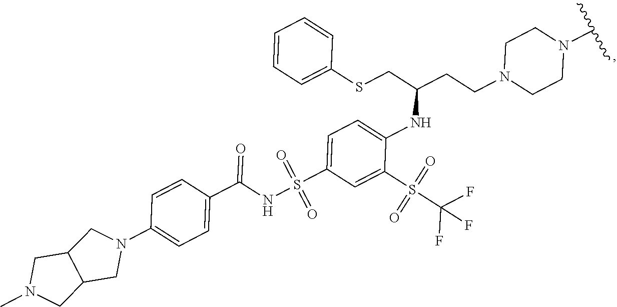 [SMILES: *N1CCN(CC[C@H](CSc2ccccc2)Nc2ccc(S(=O)(=O)NC(=O)c3ccc(N4CC5CN(C)CC5C4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

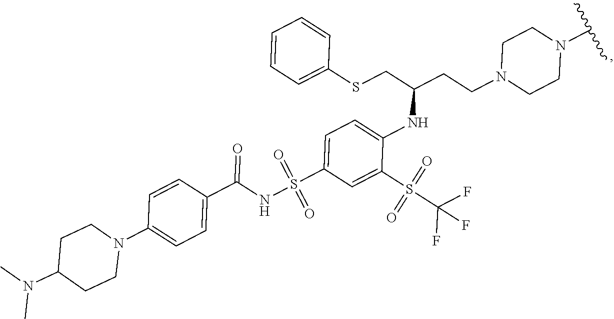 [SMILES: *N1CCN(CC[C@H](CSc2ccccc2)Nc2ccc(S(=O)(=O)NC(=O)c3ccc(N4CCC(N(C)C)CC4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

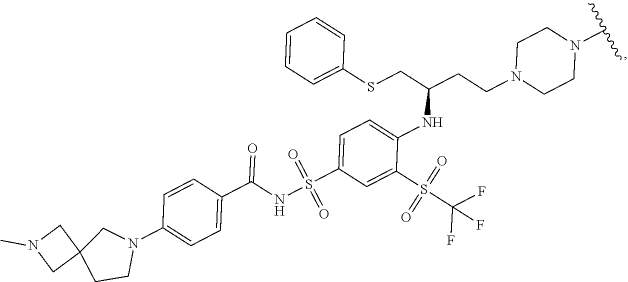 [SMILES: *N1CCN(CC[C@H](CSc2ccccc2)Nc2ccc(S(=O)(=O)NC(=O)c3ccc(N4CCC5(CN(C)C5)C4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

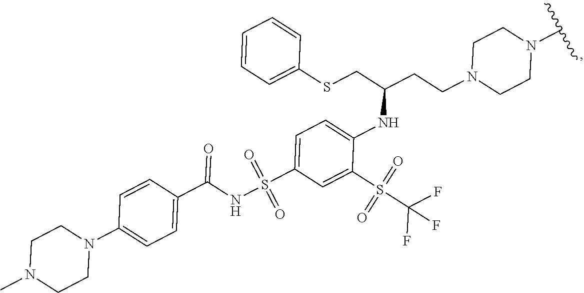 [SMILES: *N1CCN(CC[C@H](CSc2ccccc2)Nc2ccc(S(=O)(=O)NC(=O)c3ccc(N4CCN(C)CC4)cc3)cc2S(=O)(=O)C(F)(F)F)CC1]

LBM is selected from any of those in Table A below, and L is selected from any of those in Table B below.

In some embodiments, a provided compound or pharmaceutically acceptable salt thereof, is selected from those wherein BBM is

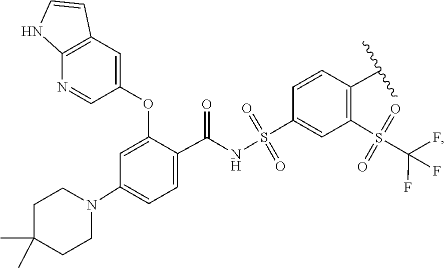 [SMILES: *c1ccc(S(=O)(=O)NC(=O)c2ccc(N3CCC(C)(C)CC3)cc2Oc2cnc3[nH]ccc3c2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

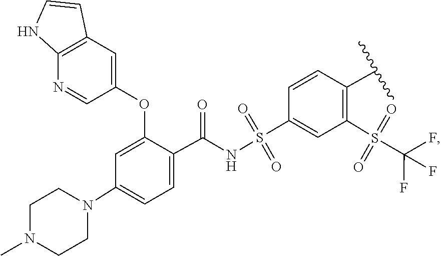 [SMILES: *c1ccc(S(=O)(=O)NC(=O)c2ccc(N3CCN(C)CC3)cc2Oc2cnc3[nH]ccc3c2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

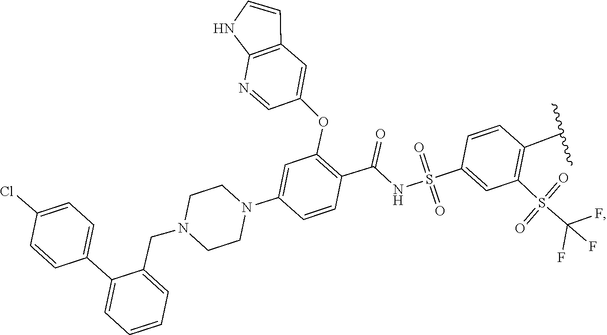 [SMILES: *c1ccc(S(=O)(=O)NC(=O)c2ccc(N3CCN(Cc4ccccc4-c4ccc(Cl)cc4)CC3)cc2Oc2cnc3[nH]ccc3c2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

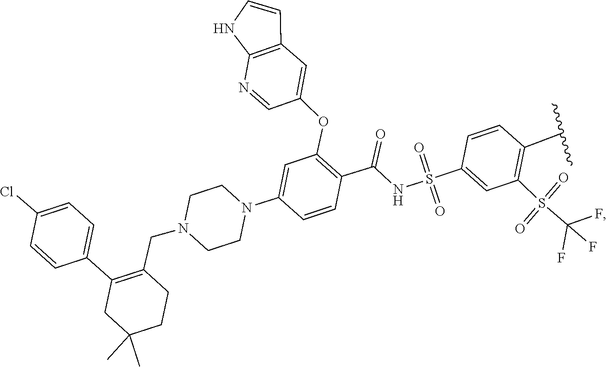 [SMILES: *c1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

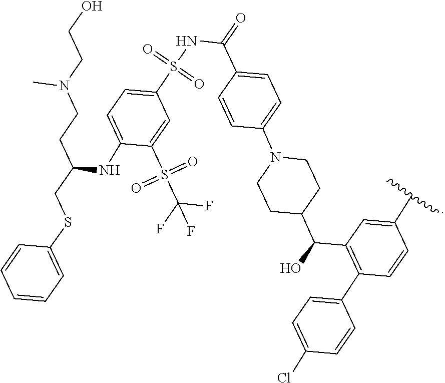 [SMILES: *c1ccc(-c2ccc(Cl)cc2)c([C@@H](O)C2CCN(c3ccc(C(=O)NS(=O)(=O)c4ccc(N[C@H](CCN(C)CCO)CSc5ccccc5)c(S(=O)(=O)C(F)(F)F)c4)cc3)CC2)c1]

LBM is selected from any of those in Table A below, and L is selected from any of those in Table B below.

In some embodiments, a provided compound or pharmaceutically acceptable salt thereof, is selected from those wherein BBM is

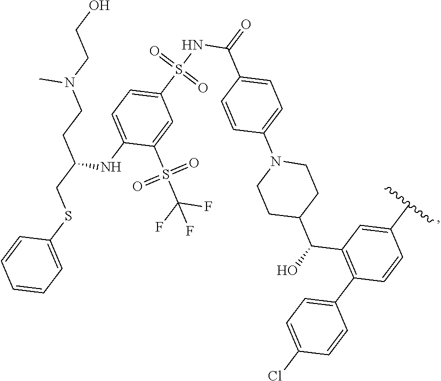 [SMILES: *c1ccc(-c2ccc(Cl)cc2)c([C@H](O)C2CCN(c3ccc(C(=O)NS(=O)(=O)c4ccc(N[C@@H](CCN(C)CCO)CSc5ccccc5)c(S(=O)(=O)C(F)(F)F)c4)cc3)CC2)c1]

LBM is selected from any of those in Table A below, and L is selected from any of those in Table B below.

In some embodiments, a provided compound or pharmaceutically acceptable salt thereof, is selected from those wherein BBM is

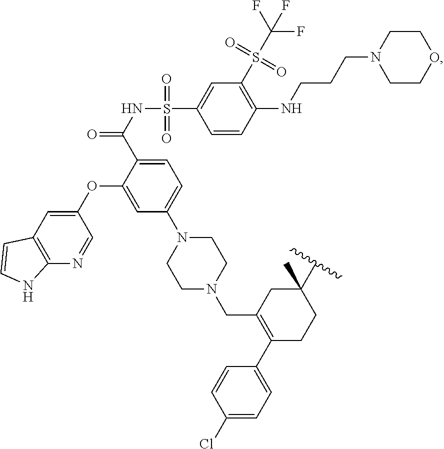 [SMILES: *[C@]1(C)CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1]

LBM is selected from any of those in Table A below, and L is selected from any of those in Table B below.

In some embodiments, a provided compound or pharmaceutically acceptable salt thereof, is selected from those wherein BBM is

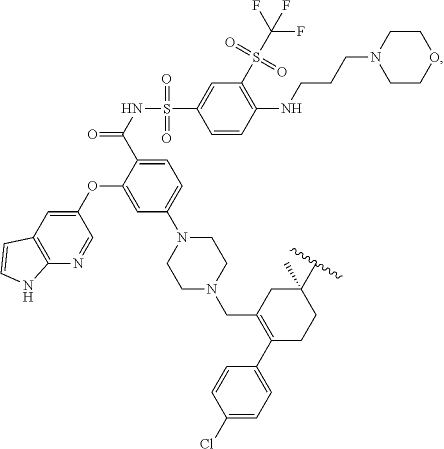 [SMILES: *[C@@]1(C)CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1]

LBM is selected from any of those in Table A below, and L is selected from any of those in Table B below.

In some embodiments, a provided compound or pharmaceutically acceptable salt thereof, is selected from those wherein BBM is

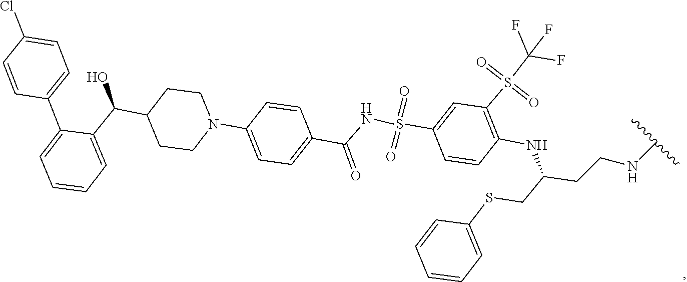 [SMILES: *NCC[C@H](CSc1ccccc1)Nc1ccc(S(=O)(=O)NC(=O)c2ccc(N3CCC([C@H](O)c4ccccc4-c4ccc(Cl)cc4)CC3)cc2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

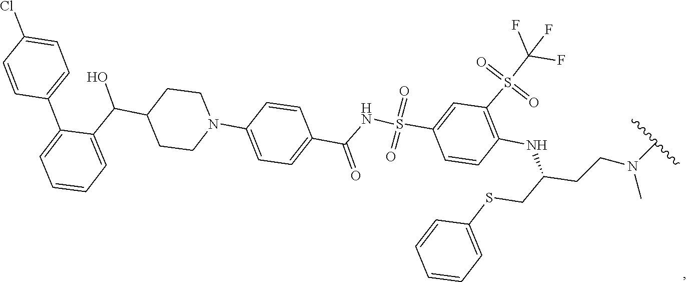 [SMILES: *N(C)CC[C@H](CSc1ccccc1)Nc1ccc(S(=O)(=O)NC(=O)c2ccc(N3CCC(C(O)c4ccccc4-c4ccc(Cl)cc4)CC3)cc2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

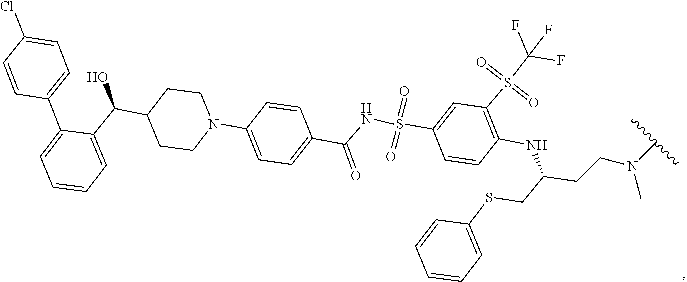 [SMILES: *N(C)CC[C@H](CSc1ccccc1)Nc1ccc(S(=O)(=O)NC(=O)c2ccc(N3CCC([C@H](O)c4ccccc4-c4ccc(Cl)cc4)CC3)cc2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

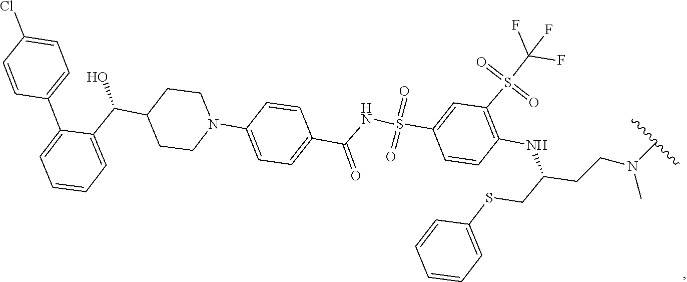 [SMILES: *N(C)CC[C@H](CSc1ccccc1)Nc1ccc(S(=O)(=O)NC(=O)c2ccc(N3CCC([C@@H](O)c4ccccc4-c4ccc(Cl)cc4)CC3)cc2)cc1S(=O)(=O)C(F)(F)F]

LBM is selected from any of those in Table A below, and L is selected from any of those in Table B below.

In some embodiments, a provided compound or pharmaceutically acceptable salt thereof, is selected from those wherein BBM is

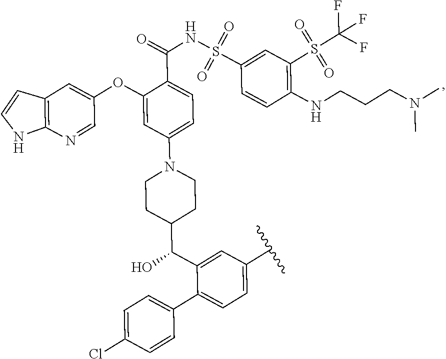 [SMILES: *c1ccc(-c2ccc(Cl)cc2)c([C@H](O)C2CCN(c3ccc(C(=O)NS(=O)(=O)c4ccc(NCCCN(C)C)c(S(=O)(=O)C(F)(F)F)c4)c(Oc4cnc5[nH]ccc5c4)c3)CC2)c1]

LBM is selected from any of those in Table A below, and L is selected from any of those in Table B below.

In some embodiments, a provided compound or pharmaceutically acceptable salt thereof, is selected from those wherein BBM is

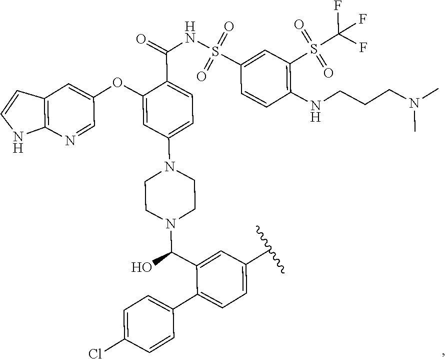 [SMILES: *c1ccc(-c2ccc(Cl)cc2)c([C@@H](O)N2CCN(c3ccc(C(=O)NS(=O)(=O)c4ccc(NCCCN(C)C)c(S(=O)(=O)C(F)(F)F)c4)c(Oc4cnc5[nH]ccc5c4)c3)CC2)c1]

LBM is selected from any of those in Table A below, and L is selected from any of those in Table B below.

TABLE A
Exemplified E3 Ligase Binding Moiety (LBM)

| | |
|---|---|
| 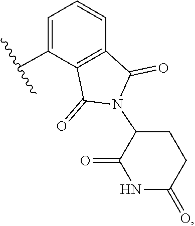 [SMILES: *c1cccc2c1C(=O)N(C1CCC(=O)NC1=O)C2=O] | (a) |
| 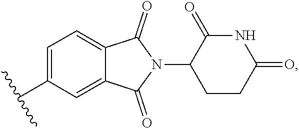 [SMILES: *c1ccc2c(c1)C(=O)N(C1CCC(=O)NC1=O)C2=O] | (b) |
| 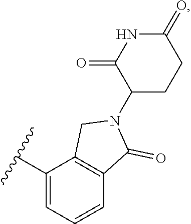 [SMILES: *c1cccc2c1CN(C1CCC(=O)NC1=O)C2=O] | (c) |
| 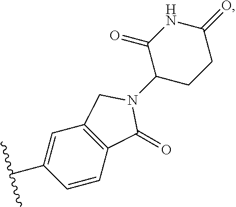 [SMILES: *c1ccc2c(c1)CN(C1CCC(=O)NC1=O)C2=O] | (d) |
| 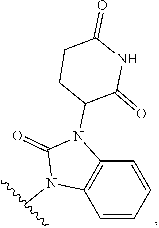 [SMILES: *n1c(=O)n(C2CCC(=O)NC2=O)c2ccccc21] | (e) |
| 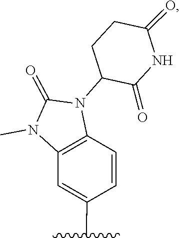 [SMILES: *c1ccc2c(c1)n(C)c(=O)n2C1CCC(=O)NC1=O] | (f) |
| 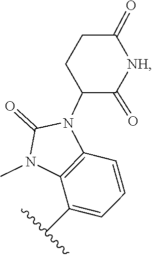 [SMILES: *c1cccc2c1n(C)c(=O)n2C1CCC(=O)NC1=O] | (g) |
| 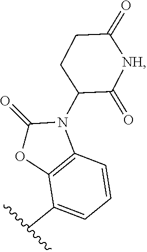 [SMILES: *c1cccc2c1oc(=O)n2C1CCC(=O)NC1=O] | (h) |
| 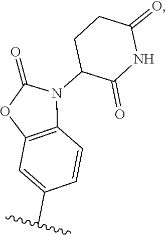 [SMILES: *c1ccc2c(c1)oc(=O)n2C1CCC(=O)NC1=O] | (i) |
| 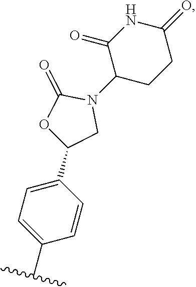 [SMILES: *c1ccc([C@H]2CN(C3CCC(=O)NC3=O)C(=O)O2)cc1] | (j) |
| 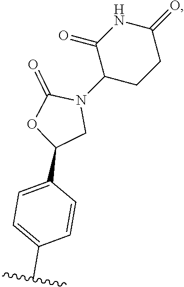 [SMILES: *c1ccc([C@@H]2CN(C3CCC(=O)NC3=O)C(=O)O2)cc1] | (k) |
| 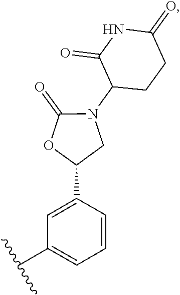 [SMILES: *c1cccc([C@H]2CN(C3CCC(=O)NC3=O)C(=O)O2)c1] | (l) |
| 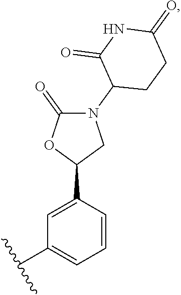 [SMILES: *c1cccc([C@@H]2CN(C3CCC(=O)NC3=O)C(=O)O2)c1] | (m) |
| 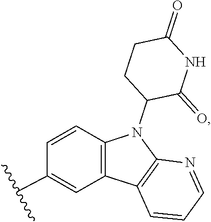 [SMILES: *c1ccc2c(c1)c1cccnc1n2C1CCC(=O)NC1=O] | (n) |
| 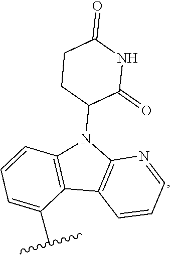 [SMILES: *c1cccc2c1c1cccnc1n2C1CCC(=O)NC1=O] | (o) |
| 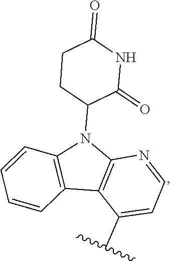 [SMILES: *c1ccnc2c1c1ccccc1n2C1CCC(=O)NC1=O] | (p) |
| 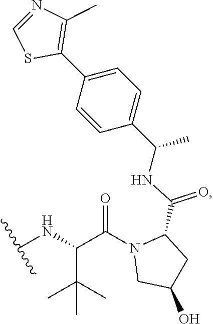 [SMILES: *N[C@H](C(=O)N1C[C@H](O)C[C@H]1C(=O)N[C@@H](C)c1ccc(-c2scnc2C)cc1)C(C)(C)C] | (q) |
| 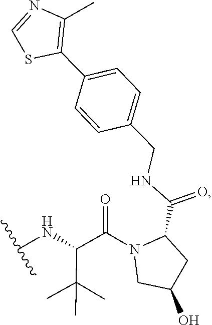 [SMILES: *N[C@H](C(=O)N1C[C@H](O)C[C@H]1C(=O)NCc1ccc(-c2scnc2C)cc1)C(C)(C)C] | (r) |
| 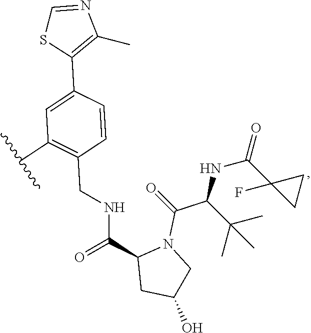 [SMILES: *c1cc(-c2scnc2C)ccc1CNC(=O)[C@@H]1C[C@@H](O)CN1C(=O)[C@@H](NC(=O)C1(F)CC1)C(C)(C)C] | (s) |
| 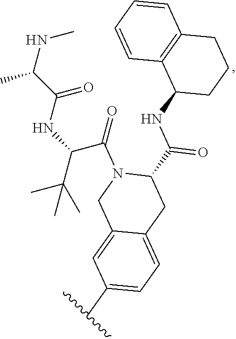 [SMILES: *c1ccc2c(c1)CN(C(=O)[C@@H](NC(=O)[C@H](C)NC)C(C)(C)C)[C@H](C(=O)N[C@@H]1CCCc3ccccc31)C2] | (t) |
| 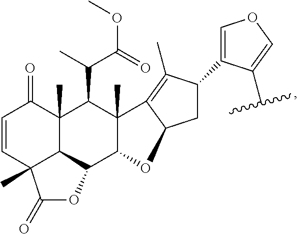 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45] | (u) |
| 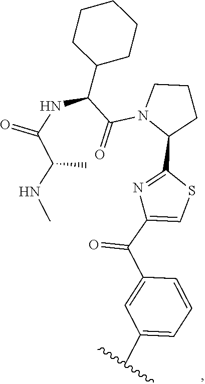 [SMILES: *c1cccc(C(=O)c2csc([C@@H]3CCCN3C(=O)[C@@H](NC(=O)[C@H](C)NC)C3CCCCC3)n2)c1] | (v) |
| 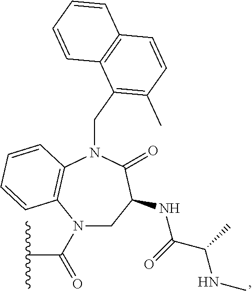 [SMILES: *C(=O)N1C[C@H](NC(=O)[C@H](C)NC)C(=O)N(Cc2c(C)ccc3ccccc23)c2ccccc21] | (w) |
| 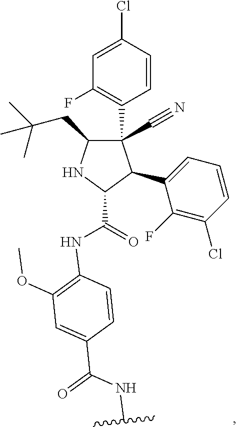 [SMILES: *NC(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1] | (x) |
| 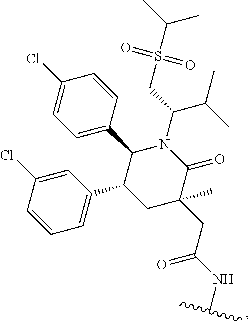 [SMILES: *NC(=O)C[C@@]1(C)C[C@H](c2cccc(Cl)c2)[C@@H](c2ccc(Cl)cc2)N([C@H](CS(=O)(=O)C(C)C)C(C)C)C1=O] | (y) |
| 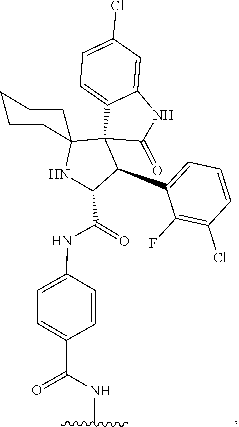 [SMILES: *NC(=O)c1ccc(NC(=O)[C@@H]2NC3(CCCCC3)[C@@]3(C(=O)Nc4cc(Cl)ccc43)[C@H]2c2cccc(Cl)c2F)cc1] | (z) |
| 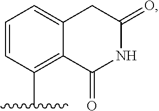 [SMILES: *c1cccc2c1C(=O)NC(=O)C2] | (aa) |
| 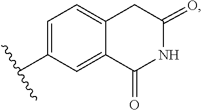 [SMILES: *c1ccc2c(c1)C(=O)NC(=O)C2] | (bb) |
| 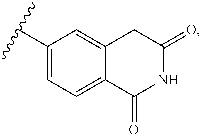 [SMILES: *c1ccc2c(c1)CC(=O)NC2=O] | (cc) |
| 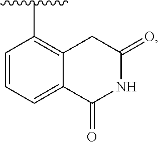 [SMILES: *c1cccc2c1CC(=O)NC2=O] | (dd) |
| 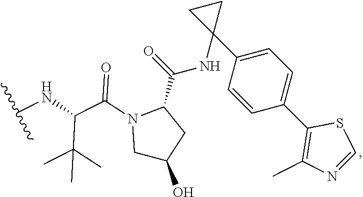 [SMILES: *N[C@H](C(=O)N1C[C@H](O)C[C@H]1C(=O)NC1(c2ccc(-c3scnc3C)cc2)CC1)C(C)(C)C] | (ee) |
| 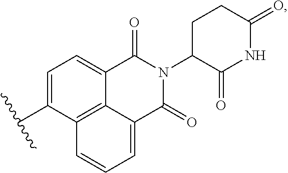 [SMILES: *c1ccc2c3c(cccc13)C(=O)N(C1CCC(=O)NC1=O)C2=O] | (ff) |
| 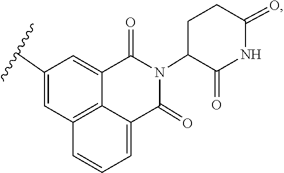 [SMILES: *c1cc2c3c(cccc3c1)C(=O)N(C1CCC(=O)NC1=O)C2=O] | (gg) |
| 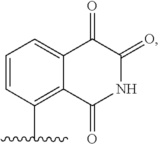 [SMILES: *c1cccc2c1C(=O)NC(=O)C2=O] | (hh) |
| 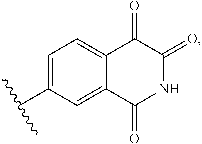 [SMILES: *c1ccc2c(c1)C(=O)NC(=O)C2=O] | (ii) |
| 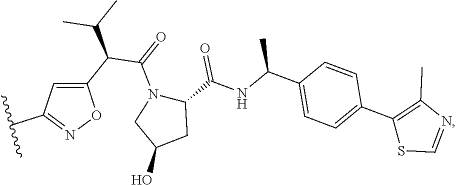 [SMILES: *c1cc([C@H](C(=O)N2C[C@H](O)C[C@H]2C(=O)N[C@@H](C)c2ccc(-c3scnc3C)cc2)C(C)C)on1] | (jj) |
| 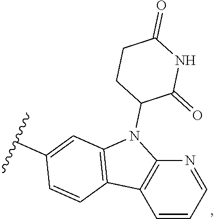 [SMILES: *c1ccc2c3cccnc3n(C3CCC(=O)NC3=O)c2c1] | (kk) |
| 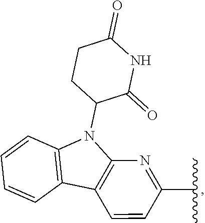 [SMILES: *c1ccc2c3ccccc3n(C3CCC(=O)NC3=O)c2n1] | (ll) |
| 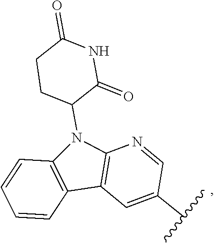 [SMILES: *c1cnc2c(c1)c1ccccc1n2C1CCC(=O)NC1=O] | (mm) |
| 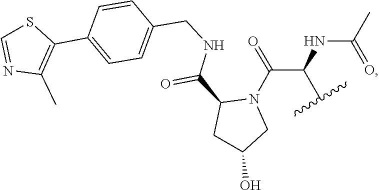 [SMILES: *[C@H](NC(C)=O)C(=O)N1C[C@H](O)C[C@H]1C(=O)NCc1ccc(-c2scnc2C)cc1] | (nn) |
| 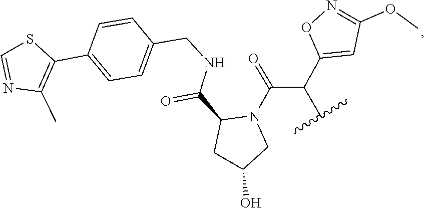 [SMILES: *C(C(=O)N1C[C@H](O)C[C@H]1C(=O)NCc1ccc(-c2scnc2C)cc1)c1cc(OC)no1] | (oo) |
| 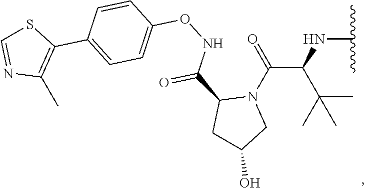 [SMILES: *N[C@H](C(=O)N1C[C@H](O)C[C@H]1C(=O)NOc1ccc(-c2scnc2C)cc1)C(C)(C)C] | (pp) |
| 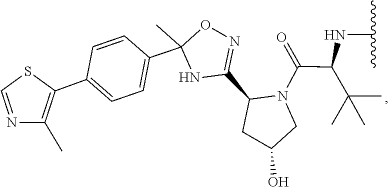 [SMILES: *N[C@H](C(=O)N1C[C@H](O)C[C@H]1C1=NOC(C)(c2ccc(-c3scnc3C)cc2)N1)C(C)(C)C] | (qq) |
| 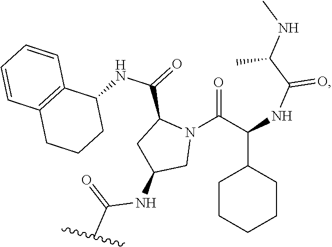 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1] | (rr) |
| 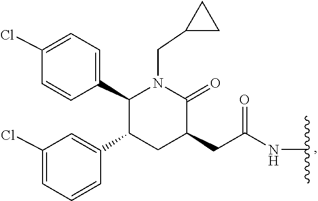 [SMILES: *NC(=O)C[C@H]1C[C@H](c2cccc(Cl)c2)[C@@H](c2ccc(Cl)cc2)N(CC2CC2)C1=O] | (ss) |
| 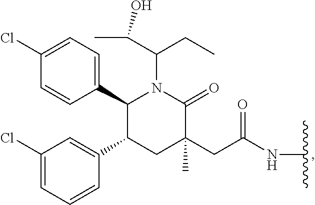 [SMILES: *NC(=O)C[C@@]1(C)C[C@H](c2cccc(Cl)c2)[C@@H](c2ccc(Cl)cc2)N(C(CC)[C@H](C)O)C1=O] | (tt) |
| 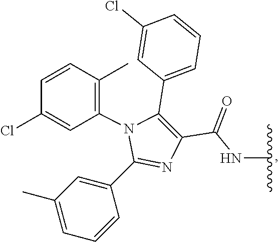 [SMILES: *NC(=O)c1nc(-c2cccc(C)c2)n(-c2cc(Cl)ccc2C)c1-c1cccc(Cl)c1] | (uu) |
| 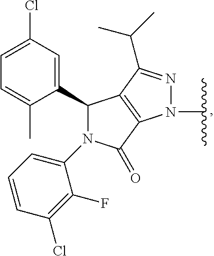 [SMILES: *n1nc(C(C)C)c2c1C(=O)N(c1cccc(Cl)c1F)[C@H]2c1cc(Cl)ccc1C] | (vv) |
| 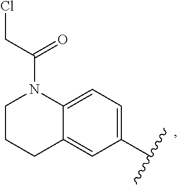 [SMILES: *c1ccc2c(c1)CCCN2C(=O)CCl] | (ww) |
| 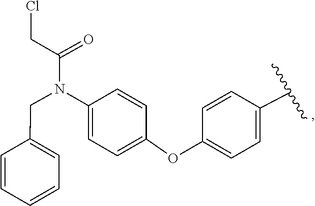 [SMILES: *c1ccc(Oc2ccc(N(Cc3ccccc3)C(=O)CCl)cc2)cc1] | (xx) |
| 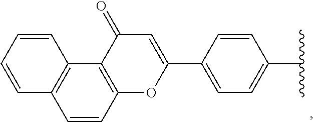 [SMILES: *c1ccc(-c2cc(=O)c3c(ccc4ccccc43)o2)cc1] | (vv) |
| 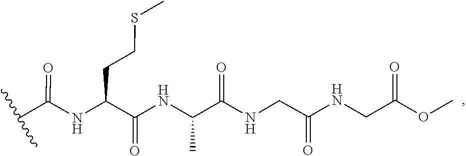 [SMILES: *C(=O)N[C@@H](CCSC)C(=O)N[C@@H](C)C(=O)NCC(=O)NCC(=O)OC] | (zz) |
| 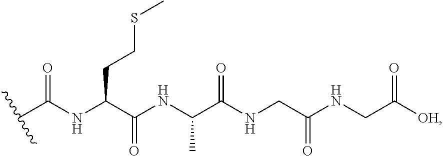 [SMILES: *C(=O)N[C@@H](CCSC)C(=O)N[C@@H](C)C(=O)NCC(=O)NCC(=O)O] | (aaa) |
| 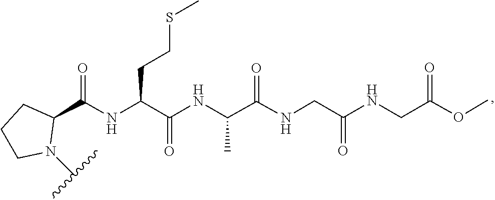 [SMILES: *N1CCC[C@H]1C(=O)N[C@@H](CCSC)C(=O)N[C@@H](C)C(=O)NCC(=O)NCC(=O)OC] | (bbb) |
| 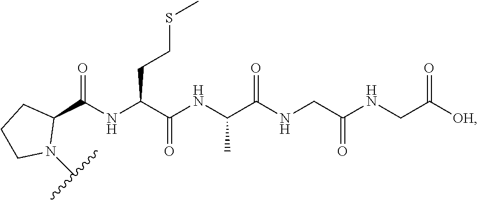 [SMILES: *N1CCC[C@H]1C(=O)N[C@@H](CCSC)C(=O)N[C@@H](C)C(=O)NCC(=O)NCC(=O)O] | (ccc) |
| 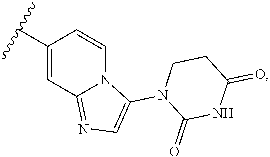 [SMILES: *c1ccn2c(N3CCC(=O)NC3=O)cnc2c1] | (ddd) |
| 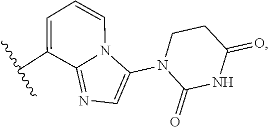 [SMILES: *c1cccn2c(N3CCC(=O)NC3=O)cnc12] | (eee) |
| 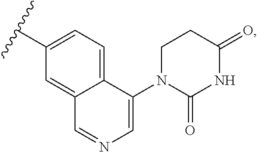 [SMILES: *c1ccc2c(N3CCC(=O)NC3=O)cncc2c1] | (fff) |
| 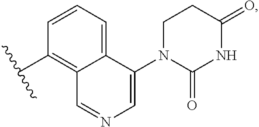 [SMILES: *c1cccc2c(N3CCC(=O)NC3=O)cncc12] | (ggg) |
| 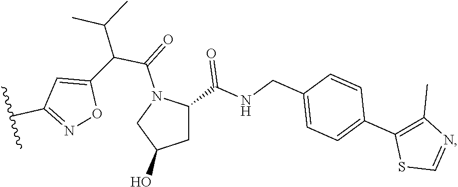 [SMILES: *c1cc(C(C(=O)N2C[C@H](O)C[C@H]2C(=O)NCc2ccc(-c3scnc3C)cc2)C(C)C)on1] | (hhh) |
| 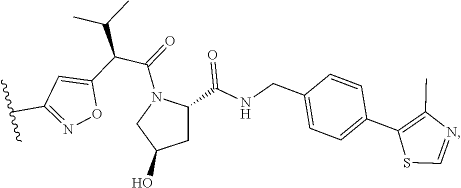 [SMILES: *c1cc([C@H](C(=O)N2C[C@H](O)C[C@H]2C(=O)NCc2ccc(-c3scnc3C)cc2)C(C)C)on1] | (iii) |
| 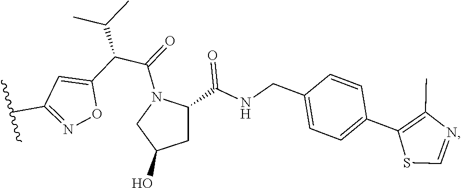 [SMILES: *c1cc([C@@H](C(=O)N2C[C@H](O)C[C@H]2C(=O)NCc2ccc(-c3scnc3C)cc2)C(C)C)on1] | (jjj) |
| 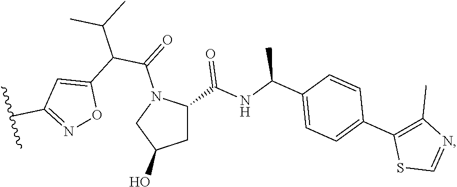 [SMILES: *c1cc(C(C(=O)N2C[C@H](O)C[C@H]2C(=O)N[C@@H](C)c2ccc(-c3scnc3C)cc2)C(C)C)on1] | (kkk) |
| 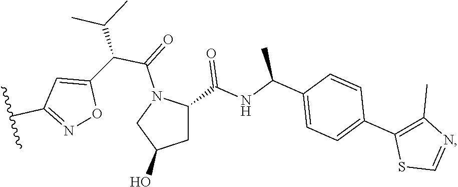 [SMILES: *c1cc([C@@H](C(=O)N2C[C@H](O)C[C@H]2C(=O)N[C@@H](C)c2ccc(-c3scnc3C)cc2)C(C)C)on1] | (lll) |
| 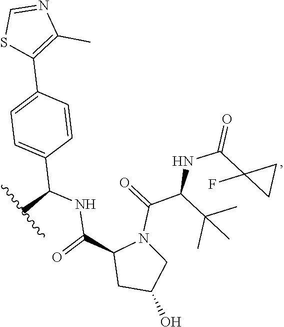 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1] | (mmm) |
| 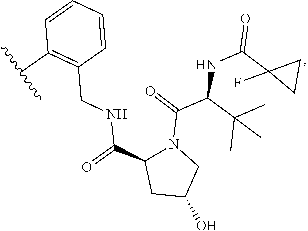 [SMILES: *c1ccccc1CNC(=O)[C@@H]1C[C@@H](O)CN1C(=O)[C@@H](NC(=O)C1(F)CC1)C(C)(C)C] | (nnn) |
| 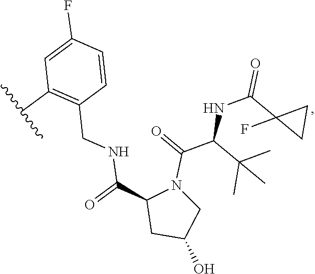 [SMILES: *c1cc(F)ccc1CNC(=O)[C@@H]1C[C@@H](O)CN1C(=O)[C@@H](NC(=O)C1(F)CC1)C(C)(C)C] | (ooo) |
| 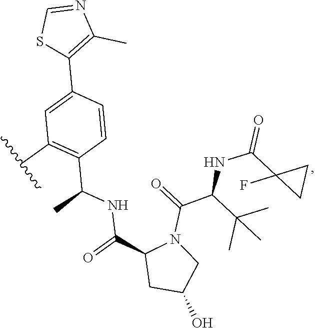 [SMILES: *c1cc(-c2scnc2C)ccc1[C@H](C)NC(=O)[C@@H]1C[C@@H](O)CN1C(=O)[C@@H](NC(=O)C1(F)CC1)C(C)(C)C] | (ppp) |
| 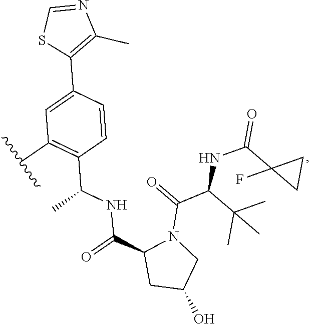 [SMILES: *c1cc(-c2scnc2C)ccc1[C@@H](C)NC(=O)[C@@H]1C[C@@H](O)CN1C(=O)[C@@H](NC(=O)C1(F)CC1)C(C)(C)C] | (qqq) |
| 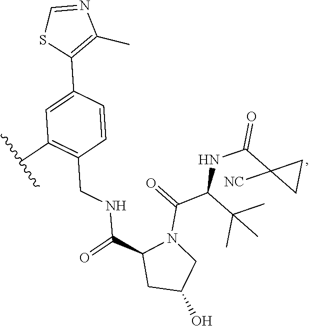 [SMILES: *c1cc(-c2scnc2C)ccc1CNC(=O)[C@@H]1C[C@@H](O)CN1C(=O)[C@@H](NC(=O)C1(C#N)CC1)C(C)(C)C] | (rrr) |
| 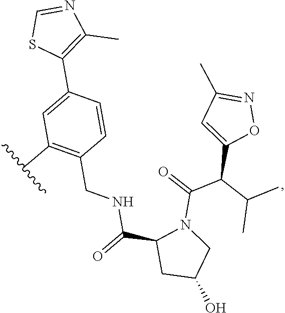 [SMILES: *c1cc(-c2scnc2C)ccc1CNC(=O)[C@@H]1C[C@@H](O)CN1C(=O)[C@@H](c1cc(C)no1)C(C)C] | (sss) |
| 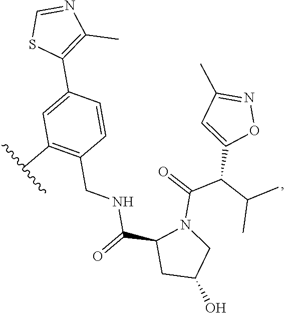 [SMILES: *c1cc(-c2scnc2C)ccc1CNC(=O)[C@@H]1C[C@@H](O)CN1C(=O)[C@H](c1cc(C)no1)C(C)C] | (ttt) |
| 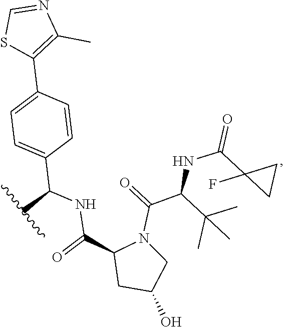 [SMILES: *[C@H](NC(=O)[C@@H]1C[C@@H](O)CN1C(=O)[C@@H](NC(=O)C1(F)CC1)C(C)(C)C)c1ccc(-c2scnc2C)cc1] | (uuu) |
| 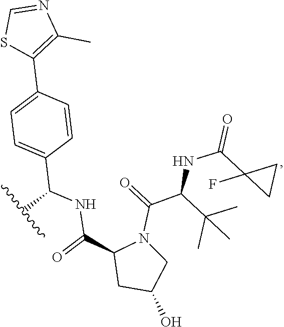 [SMILES: *[C@@H](NC(=O)[C@@H]1C[C@@H](O)CN1C(=O)[C@@H](NC(=O)C1(F)CC1)C(C)(C)C)c1ccc(-c2scnc2C)cc1] | (vvv) |
| 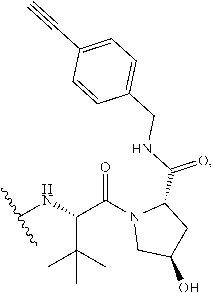 [SMILES: *N[C@H](C(=O)N1C[C@H](O)C[C@H]1C(=O)NCc1ccc(C#C)cc1)C(C)(C)C] | (www) |
| 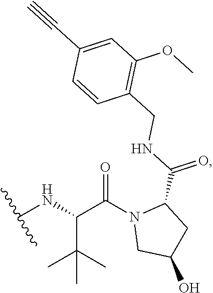 [SMILES: *N[C@H](C(=O)N1C[C@H](O)C[C@H]1C(=O)NCc1ccc(C#C)cc1OC)C(C)(C)C] | (xxx) |
| 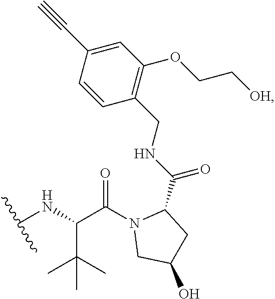 [SMILES: *N[C@H](C(=O)N1C[C@H](O)C[C@H]1C(=O)NCc1ccc(C#C)cc1OCCO)C(C)(C)C] | (yyy) |
| 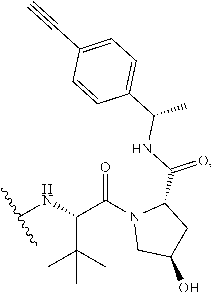 [SMILES: *N[C@H](C(=O)N1C[C@H](O)C[C@H]1C(=O)N[C@@H](C)c1ccc(C#C)cc1)C(C)(C)C] | (zzz) |
| 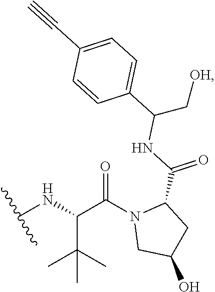 [SMILES: *N[C@H](C(=O)N1C[C@H](O)C[C@H]1C(=O)NC(CO)c1ccc(C#C)cc1)C(C)(C)C] | (aaaa) |
| 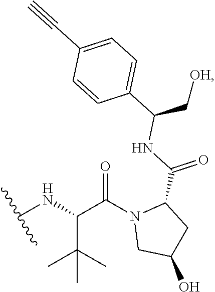 [SMILES: *N[C@H](C(=O)N1C[C@H](O)C[C@H]1C(=O)N[C@H](CO)c1ccc(C#C)cc1)C(C)(C)C] | (bbbb) |
| 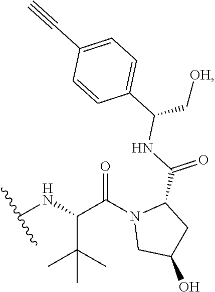 [SMILES: *N[C@H](C(=O)N1C[C@H](O)C[C@H]1C(=O)N[C@@H](CO)c1ccc(C#C)cc1)C(C)(C)C] | (cccc) |
| 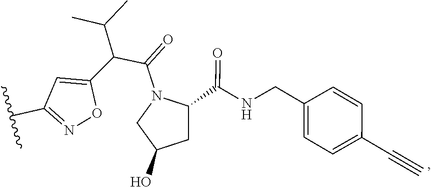 [SMILES: *c1cc(C(C(=O)N2C[C@H](O)C[C@H]2C(=O)NCc2ccc(C#C)cc2)C(C)C)on1] | (dddd) |
| 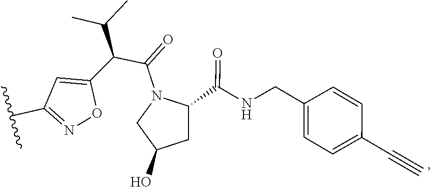 [SMILES: *c1cc([C@H](C(=O)N2C[C@H](O)C[C@H]2C(=O)NCc2ccc(C#C)cc2)C(C)C)on1] | (eeee) |
| 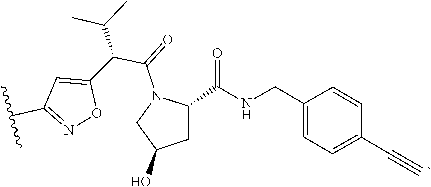 [SMILES: *c1cc([C@@H](C(=O)N2C[C@H](O)C[C@H]2C(=O)NCc2ccc(C#C)cc2)C(C)C)on1] | (ffff) |
| 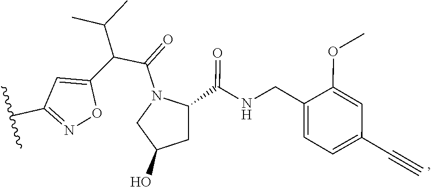 [SMILES: *c1cc(C(C(=O)N2C[C@H](O)C[C@H]2C(=O)NCc2ccc(C#C)cc2OC)C(C)C)on1] | (gggg) |
| 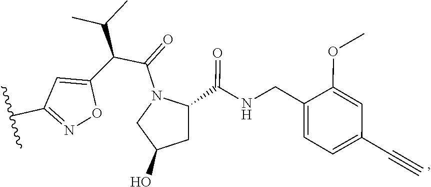 [SMILES: *c1cc([C@H](C(=O)N2C[C@H](O)C[C@H]2C(=O)NCc2ccc(C#C)cc2OC)C(C)C)on1] | (hhhh) |
| 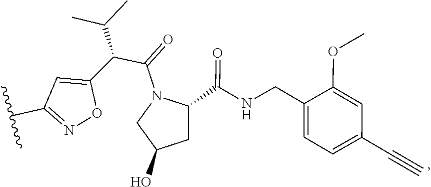 [SMILES: *c1cc([C@@H](C(=O)N2C[C@H](O)C[C@H]2C(=O)NCc2ccc(C#C)cc2OC)C(C)C)on1] | (iiii) |
| 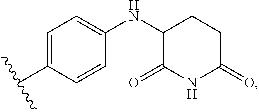 [SMILES: *c1ccc(NC2CCC(=O)NC2=O)cc1] | (jjjj) |
| 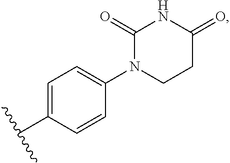 [SMILES: *c1ccc(N2CCC(=O)NC2=O)cc1] | (kkkk) |
| 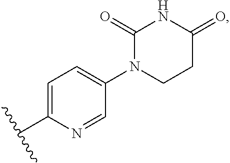 [SMILES: *c1ccc(N2CCC(=O)NC2=O)cn1] | (llll) |
| 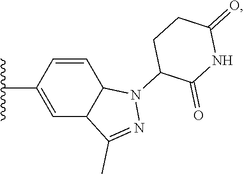 [SMILES: *C1=CC2C(C)=NN(C3CCC(=O)NC3=O)C2C=C1] | (mmmm) |
| 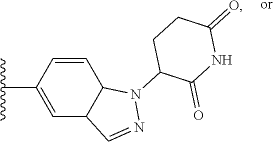 [SMILES: *C1=CC2C=NN(C3CCC(=O)NC3=O)C2C=C1 ; "or"] | (nnnn) |
| 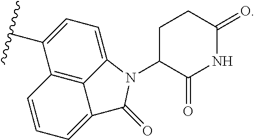 [SMILES: *c1ccc2c3c(cccc13)C(=O)N2C1CCC(=O)NC1=O] | (oooo) |

TABLE B
Exemplified Linkers (L)

| | |
|---|---|
| 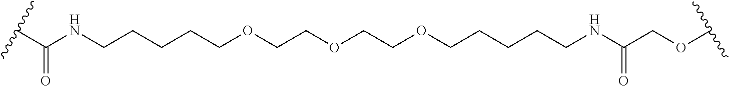 [SMILES: *OCC(=O)NCCCCCOCCOCCOCCCCCNC(*)=O] | (1) |
| 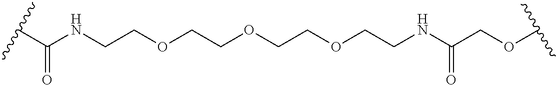 [SMILES: *OCC(=O)NCCOCCOCCOCCNC(*)=O] | (2) |
| 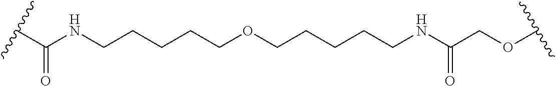 [SMILES: *OCC(=O)NCCCCCOCCCCCNC(*)=O] | (3) |
| 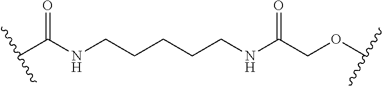 [SMILES: *OCC(=O)NCCCCCNC(*)=O] | (4) |
| 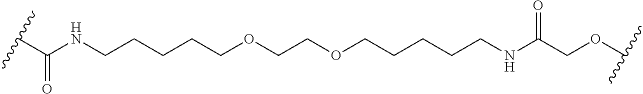 [SMILES: *OCC(=O)NCCCCCOCCOCCCCCNC(*)=O] | (5) |
| 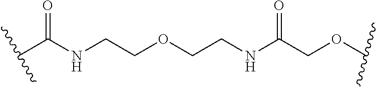 [SMILES: *OCC(=O)NCCOCCNC(*)=O] | (6) |
| 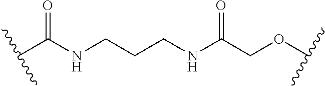 [SMILES: *OCC(=O)NCCCNC(*)=O] | (7) |
| 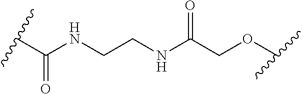 [SMILES: *OCC(=O)NCCNC(*)=O] | (8) |
| 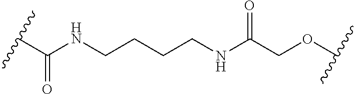 [SMILES: *OCC(=O)NCCCCNC(*)=O] | (9) |
| 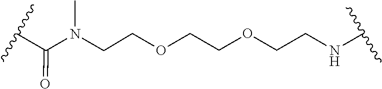 [SMILES: *NCCOCCOCCN(C)C(*)=O] | (10) |
| 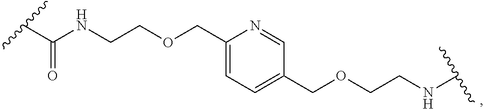 [SMILES: *NCCOCc1ccc(COCCNC(*)=O)nc1] | (11) |
| 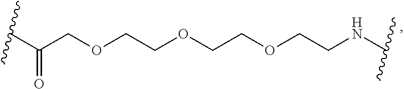 [SMILES: *NCCOCCOCCOCC(*)=O] | (12) |
| 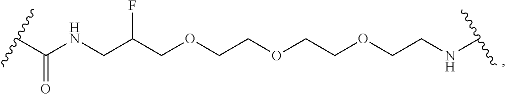 [SMILES: *NCCOCCOCCOCC(F)CNC(*)=O] | (13) |
| 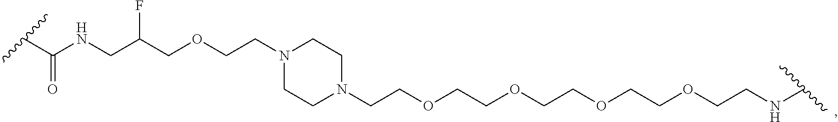 [SMILES: *NCCOCCOCCOCCOCCN1CCN(CCOCC(F)CNC(*)=O)CC1] | (14) |
| 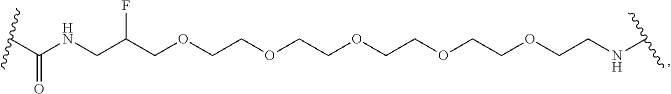 [SMILES: *NCCOCCOCCOCCOCCOCC(F)CNC(*)=O] | (15) |
| 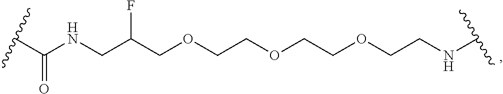 [SMILES: *NCCOCCOCCOCC(F)CNC(*)=O] | (16) |
| 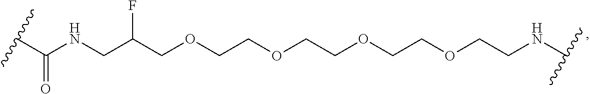 [SMILES: *NCCOCCOCCOCCOCC(F)CNC(*)=O] | (17) |
| 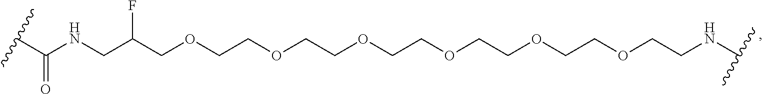 [SMILES: *NCCOCCOCCOCCOCCOCCOCC(F)CNC(*)=O] | (18) |
| 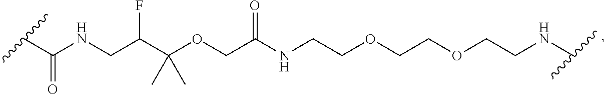 [SMILES: *NCCOCCOCCNC(=O)COC(C)(C)C(F)CNC(*)=O] | (19) |
| 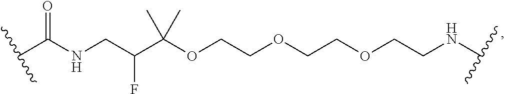 [SMILES: *NCCOCCOCCOC(C)(C)C(F)CNC(*)=O] | (20) |
| 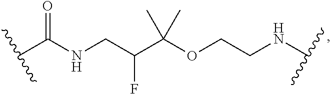 [SMILES: *NCCOC(C)(C)C(F)CNC(*)=O] | (21) |
| 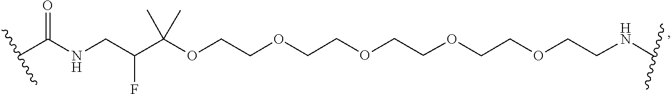 [SMILES: *NCCOCCOCCOCCOCCOC(C)(C)C(F)CNC(*)=O] | (22) |
| 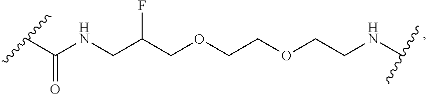 [SMILES: *NCCOCCOCC(F)CNC(*)=O] | (23) |
| 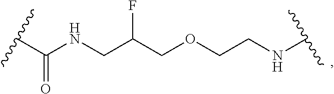 [SMILES: *NCCOCC(F)CNC(*)=O] | (24) |
| 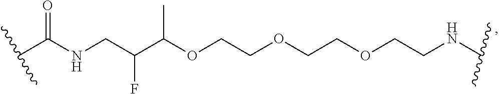 [SMILES: *NCCOCCOCCOC(C)C(F)CNC(*)=O] | (25) |
| 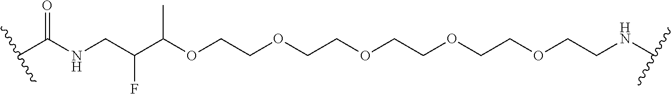 [SMILES: *NCCOCCOCCOCCOCCOC(C)C(F)CNC(*)=O] | (26) |
| 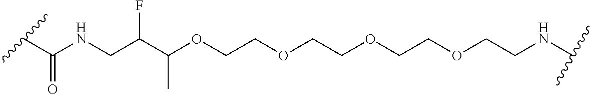 [SMILES: *NCCOCCOCCOCCOC(C)C(F)CNC(*)=O] | (27) |
| 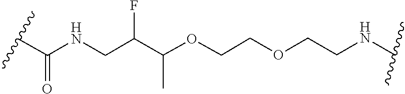 [SMILES: *NCCOCCOC(C)C(F)CNC(*)=O] | (28) |
| 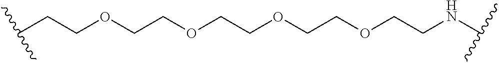 [SMILES: *CCOCCOCCOCCOCCN*] | (29) |
| 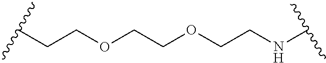 [SMILES: *CCOCCOCCN*] | (30) |
| 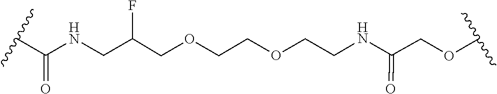 [SMILES: *OCC(=O)NCCOCCOCC(F)CNC(*)=O] | (31) |
| 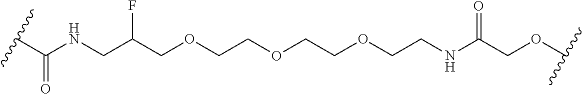 [SMILES: *OCC(=O)NCCOCCOCCOCC(F)CNC(*)=O] | (32) |
| 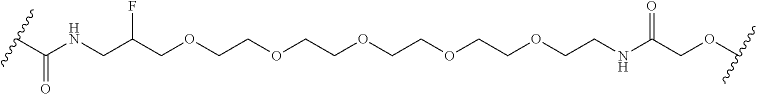 [SMILES: *OCC(=O)NCCOCCOCCOCCOCCOCC(F)CNC(*)=O] | (33) |
| 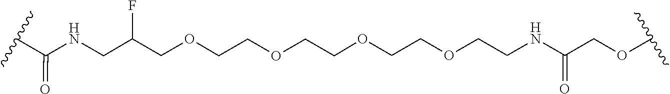 [SMILES: *OCC(=O)NCCOCCOCCOCCOCC(F)CNC(*)=O] | (34) |
| 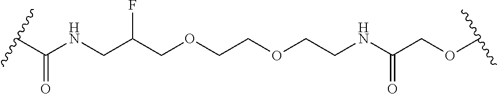 [SMILES: *OCC(=O)NCCOCCOCC(F)CNC(*)=O] | (35) |
| 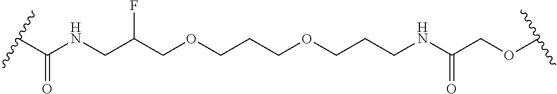 [SMILES: *OCC(=O)NCCCOCCCOCC(F)CNC(*)=O] | (36) |
| 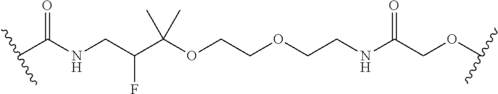 [SMILES: *OCC(=O)NCCOCCOC(C)(C)C(F)CNC(*)=O] | (37) |
| 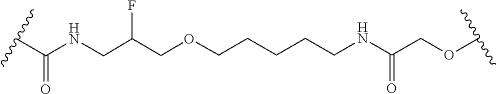 [SMILES: *OCC(=O)NCCCCCOCC(F)CNC(*)=O] | (38) |
| 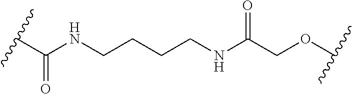 [SMILES: *OCC(=O)NCCCCNC(*)=O] | (39) |
| 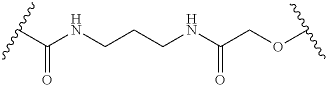 [SMILES: *OCC(=O)NCCCNC(*)=O] | (40) |
| 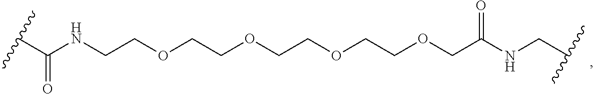 [SMILES: *CNC(=O)COCCOCCOCCOCCNC(*)=O] | (41) |
| 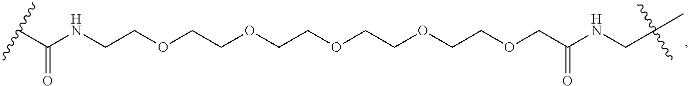 [SMILES: *CNC(=O)COCCOCCOCCOCCOCCNC(*)=O] | (42) |
| 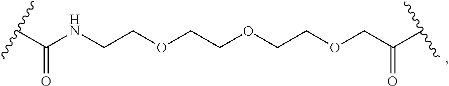 [SMILES: *C(=O)COCCOCCOCCNC(*)=O] | (43) |
| 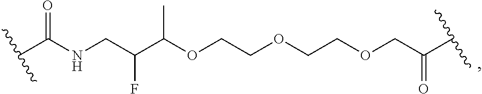 [SMILES: *C(=O)COCCOCCOC(C)C(F)CNC(*)=O] | (44) |
| 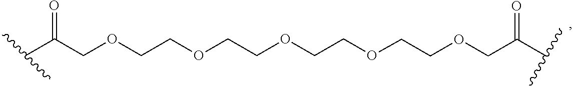 [SMILES: *C(=O)COCCOCCOCCOCCOCC(*)=O] | (45) |
| 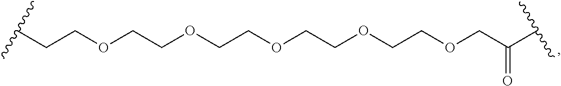 [SMILES: *CCOCCOCCOCCOCCOCC(*)=O] | (46) |
| 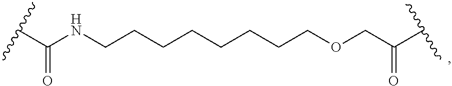 [SMILES: *C(=O)COCCCCCCCCNC(*)=O] | (47) |
| 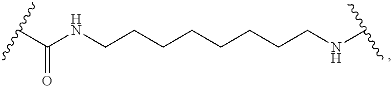 [SMILES: *NCCCCCCCCNC(*)=O] | (49) |
| 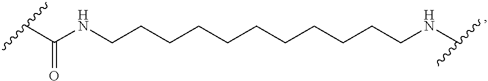 [SMILES: *NCCCCCCCCCCCNC(*)=O] | (50) |
| 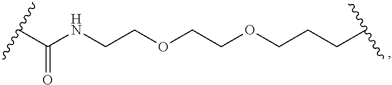 [SMILES: *CCCOCCOCCNC(*)=O] | (51) |
| 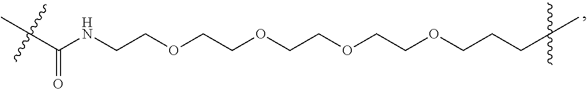 [SMILES: *CCCOCCOCCOCCOCCNC(*)=O] | (52) |
| 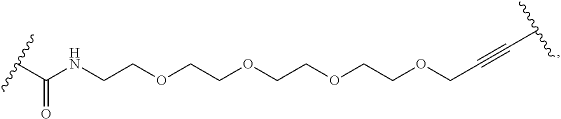 [SMILES: *C#CCOCCOCCOCCOCCNC(*)=O] | (53) |
| 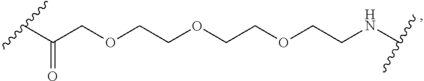 [SMILES: *NCCOCCOCCOCC(*)=O] | (54) |
| 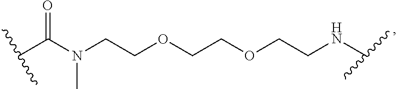 [SMILES: *NCCOCCOCCN(C)C(*)=O] | (55) |
| 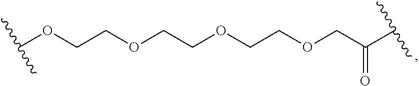 [SMILES: *OCCOCCOCCOCC(*)=O ; ","] | (56) |
| 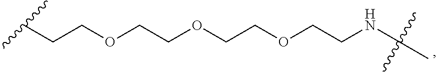 [SMILES: *CCOCCOCCOCCN*] | (57) |
| 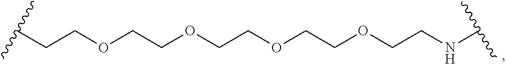 [SMILES: *CCOCCOCCOCCOCCN*] | (58) |
| 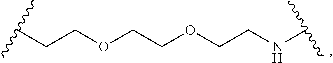 [SMILES: *CCOCCOCCN*] | (59) |
| 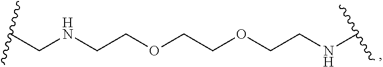 [SMILES: *CNCCOCCOCCN*] | (60) |
| 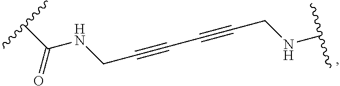 [SMILES: *NCC#CC#CCNC(*)=O] | (61) |
| 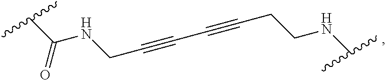 [SMILES: *NCCC#CC#CCNC(*)=O] | (62) |
| 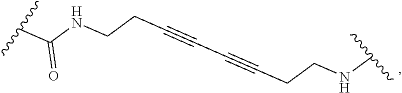 [SMILES: *NCCC#CC#CCCNC(*)=O] | (63) |
| 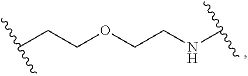 [SMILES: *CCOCCN*] | (64) |
| 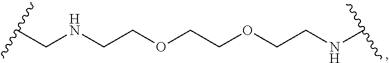 [SMILES: *CNCCOCCOCCN*] | (65) |
| 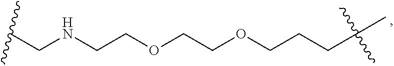 [SMILES: *CCCOCCOCCNC*] | (66) |
| 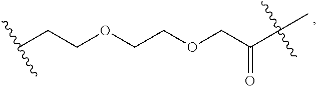 [SMILES: *CCOCCOCC(*)=O] | (67) |
| 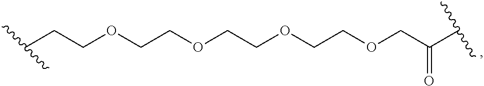 [SMILES: *CCOCCOCCOCCOCC(*)=O] | (68) |
| 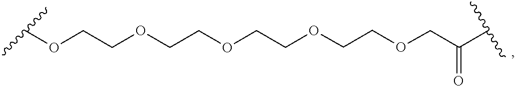 [SMILES: *OCCOCCOCCOCCOCC(*)=O] | (69) |
| 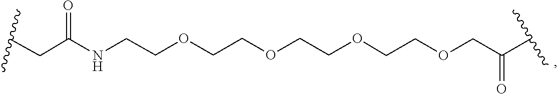 [SMILES: *CC(=O)NCCOCCOCCOCCOCC(*)=O] | (70) |
| 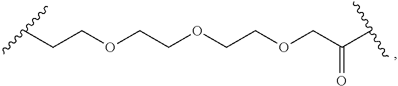 [SMILES: *CCOCCOCCOCC(*)=O] | (71) |
| 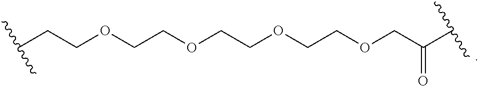 [SMILES: *CCOCCOCCOCCOCC(*)=O ; "."] | (72) |
| 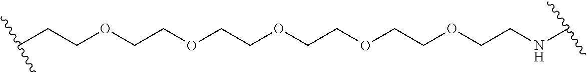 [SMILES: *CCOCCOCCOCCOCCOCCN*] | (73) |
| 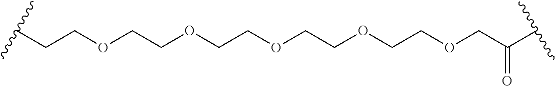 [SMILES: *CCOCCOCCOCCOCCOCC(*)=O] | (74) |
| 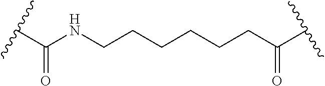 [SMILES: *C(=O)CCCCCCNC(*)=O] | (75) |
| 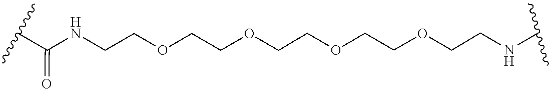 [SMILES: *NCCOCCOCCOCCOCCNC(*)=O] | (76) |
| 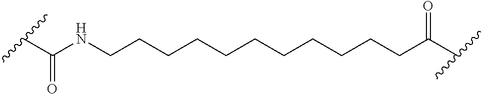 [SMILES: *C(=O)CCCCCCCCCCCNC(*)=O] | (77) |
| 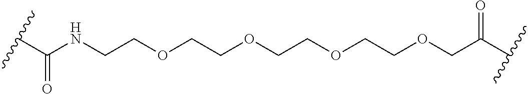 [SMILES: *C(=O)COCCOCCOCCOCCNC(*)=O] | (78) |
| 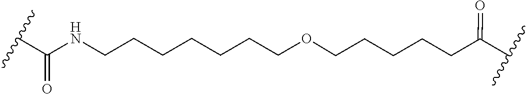 [SMILES: *C(=O)CCCCCOCCCCCCCNC(*)=O] | (79) |
| 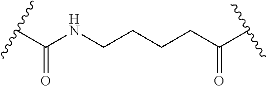 [SMILES: *C(=O)CCCCNC(*)=O] | (80) |
| 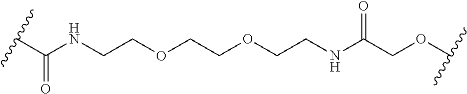 [SMILES: *OCC(=O)NCCOCCOCCNC(*)=O] | (81) |
| 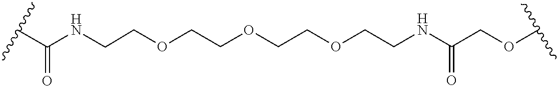 [SMILES: *OCC(=O)NCCOCCOCCOCCNC(*)=O] | (82) |
| 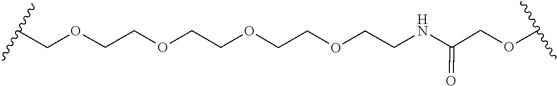 [SMILES: *COCCOCCOCCOCCNC(=O)CO*] | (83) |
| 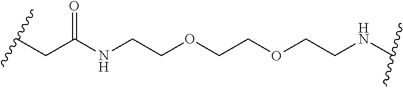 [SMILES: *CC(=O)NCCOCCOCCN*] | (84) |
| 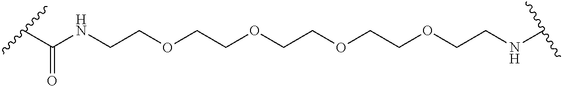 [SMILES: *NCCOCCOCCOCCOCCNC(*)=O] | (85) |
| 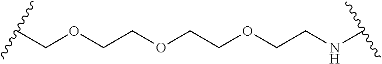 [SMILES: *COCCOCCOCCN*] | (86) |
| 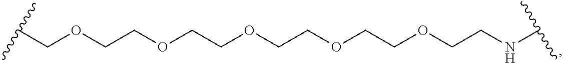 [SMILES: *COCCOCCOCCOCCOCCN*] | (87) |
| 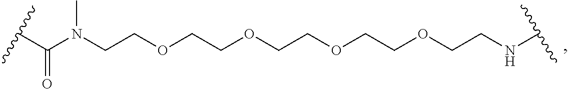 [SMILES: *NCCOCCOCCOCCOCCN(C)C(*)=O] | (88) |
| 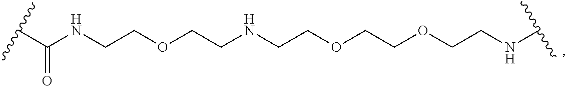 [SMILES: *NCCOCCOCCNCCOCCNC(*)=O] | (89) |
| 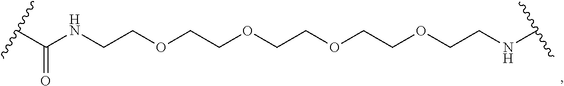 [SMILES: *NCCOCCOCCOCCOCCNC(*)=O] | (90) |
| 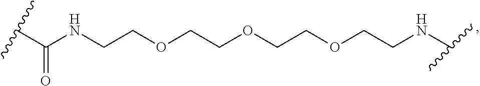 [SMILES: *NCCOCCOCCOCCNC(*)=O] | (91) |
| 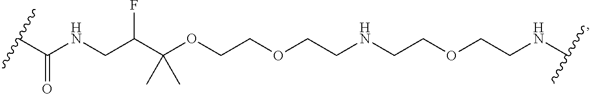 [SMILES: *NCCOCCNCCOCCOC(C)(C)C(F)CNC(*)=O ; ","] | (92) |
| 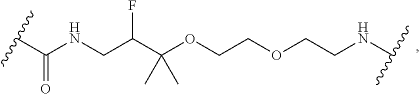 [SMILES: *NCCOCCOC(C)(C)C(F)CNC(*)=O] | (93) |
| 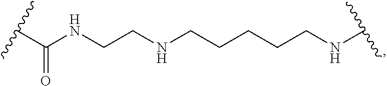 [SMILES: *NCCCCCNCCNC(*)=O] | (94) |
| 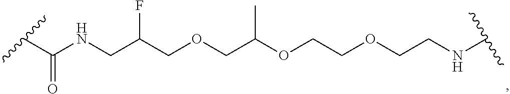 [SMILES: *NCCOCCOC(C)COCC(F)CNC(*)=O] | (95) |
| 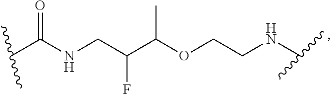 [SMILES: *NCCOC(C)C(F)CNC(*)=O] | (96) |
| 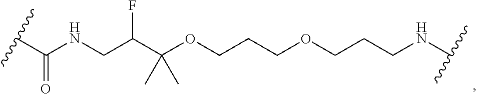 [SMILES: *NCCCOCCCOC(C)(C)C(F)CNC(*)=O] | (97) |
| 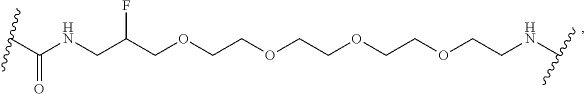 [SMILES: *NCCOCCOCCOCCOCC(F)CNC(*)=O] | (98) |
| 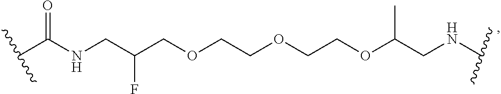 [SMILES: *NCC(C)OCCOCCOCC(F)CNC(*)=O] | (99) |
| 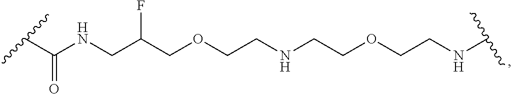 [SMILES: *NCCOCCNCCOCC(F)CNC(*)=O] | (100) |
| 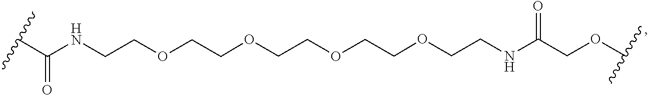 [SMILES: *OCC(=O)NCCOCCOCCOCCOCCNC(*)=O] | (101) |
| 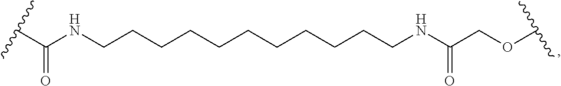 [SMILES: *OCC(=O)NCCCCCCCCCCCNC(*)=O] | (102) |
| 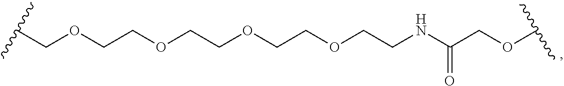 [SMILES: *COCCOCCOCCOCCNC(=O)CO*] | (103) |
| 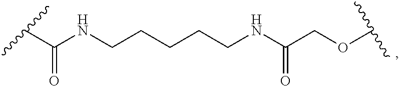 [SMILES: *OCC(=O)NCCCCCNC(*)=O] | (104) |
| 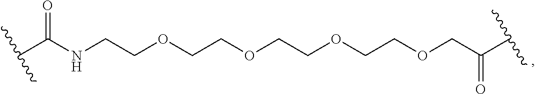 [SMILES: *C(=O)COCCOCCOCCOCCNC(*)=O] | (105) |
| 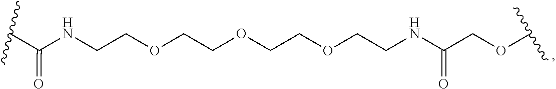 [SMILES: *OCC(=O)NCCOCCOCCOCCNC(*)=O] | (106) |
| 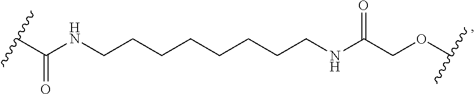 [SMILES: *OCC(=O)NCCCCCCCCNC(*)=O] | (107) |
| 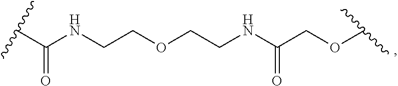 [SMILES: *OCC(=O)NCCOCCNC(*)=O] | (108) |
| 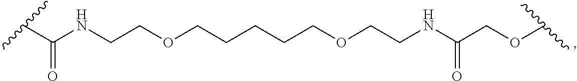 [SMILES: *OCC(=O)NCCOCCCCCOCCNC(*)=O] | (109) |
| 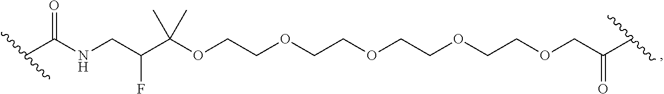 [SMILES: *C(=O)COCCOCCOCCOCCOC(C)(C)C(F)CNC(*)=O] | (110) |
| 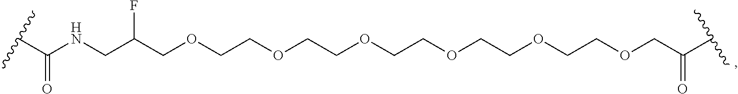 [SMILES: *C(=O)COCCOCCOCCOCCOCCOCC(F)CNC(*)=O] | (111) |
| 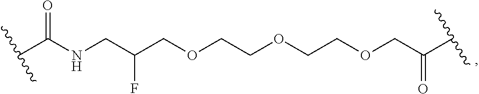 [SMILES: *C(=O)COCCOCCOCC(F)CNC(*)=O] | (112) |
| 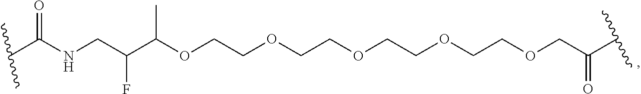 [SMILES: *C(=O)COCCOCCOCCOCCOC(C)C(F)CNC(*)=O] | (113) |
| 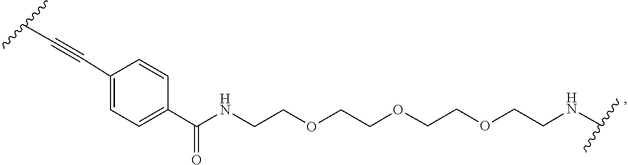 [SMILES: *C#Cc1ccc(C(=O)NCCOCCOCCOCCN*)cc1] | (114) |
| 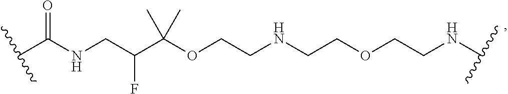 [SMILES: *NCCOCCNCCOC(C)(C)C(F)CNC(*)=O] | (115) |
| 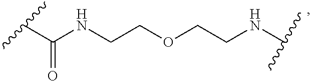 [SMILES: *NCCOCCNC(*)=O] | (116) |
| 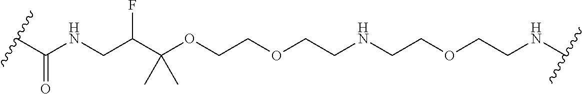 [SMILES: *NCCOCCNCCOCCOC(C)(C)C(F)CNC(*)=O] | (117) |
| 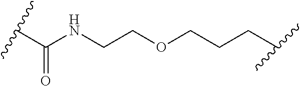 [SMILES: *CCCOCCNC(*)=O] | (118) |
| 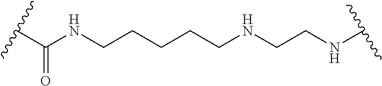 [SMILES: *NCCNCCCCCNC(*)=O] | |
| 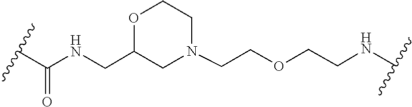 [SMILES: *NCCOCCN1CCOC(CNC(*)=O)C1] | (119) |
| 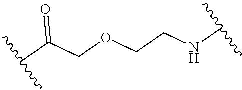 [SMILES: *NCCOCC(*)=O] | |
| 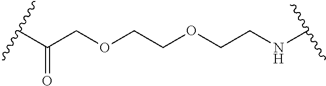 [SMILES: *NCCOCCOCC(*)=O] | (120) |
| 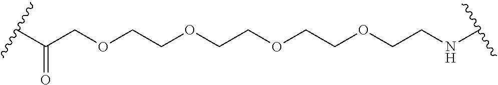 [SMILES: *NCCOCCOCCOCCOCC(*)=O] | (121) |
| 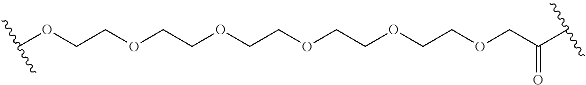 [SMILES: *OCCOCCOCCOCCOCCOCC(*)=O] | (122) |
| 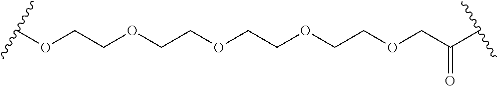 [SMILES: *OCCOCCOCCOCCOCC(*)=O] | (123) |
| 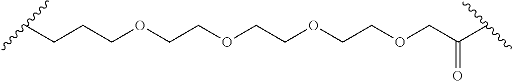 [SMILES: *CCCOCCOCCOCCOCC(*)=O] | (124) |
| 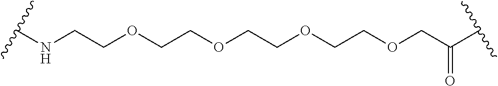 [SMILES: *NCCOCCOCCOCCOCC(*)=O] | (125) |
| 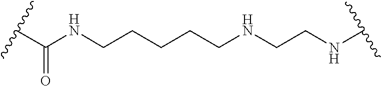 [SMILES: *NCCNCCCCCNC(*)=O] | (126) |
| 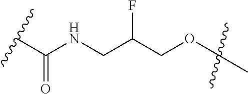 [SMILES: *OCC(F)CNC(*)=O] | |
| 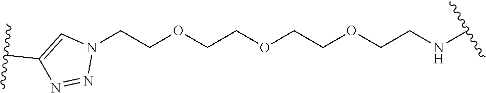 [SMILES: *NCCOCCOCCOCCn1cc(*)nn1] | (127) |
| 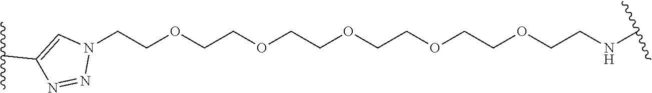 [SMILES: *NCCOCCOCCOCCOCCOCCn1cc(*)nn1] | (128) |
| 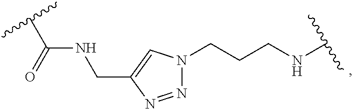 [SMILES: *NCCCn1cc(CNC(*)=O)nn1] | (129) |
| 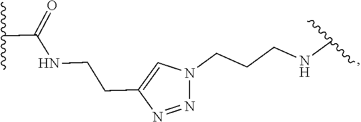 [SMILES: *NCCCn1cc(CCNC(*)=O)nn1] | (130) |
| 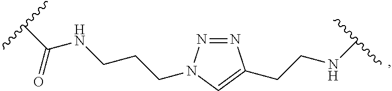 [SMILES: *NCCc1cn(CCCNC(*)=O)nn1] | (131) |
| 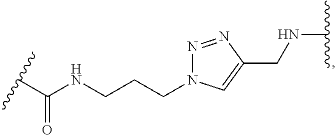 [SMILES: *NCc1cn(CCCNC(*)=O)nn1] | (132) |
| 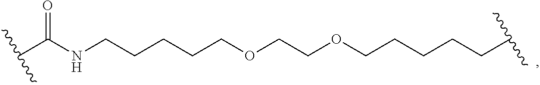 [SMILES: *CCCCCOCCOCCCCCNC(*)=O] | (133) |
| 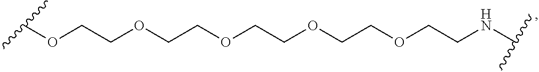 [SMILES: *NCCOCCOCCOCCOCCO*] | (134) |
| 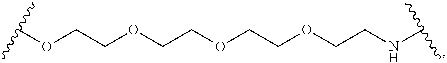 [SMILES: *NCCOCCOCCOCCO*] | (135) |
| 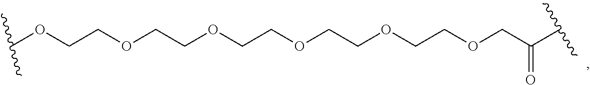 [SMILES: *OCCOCCOCCOCCOCCOCC(*)=O] | (136) |
| 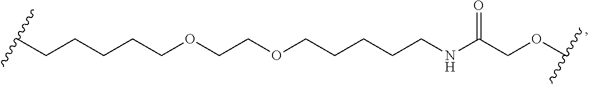 [SMILES: *CCCCCOCCOCCCCCNC(=O)CO*] | (137) |
| 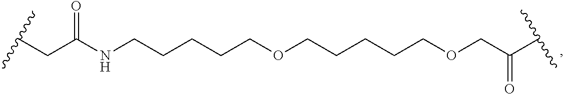 [SMILES: *CC(=O)NCCCCCOCCCCCOCC(*)=O] | (138) |
| 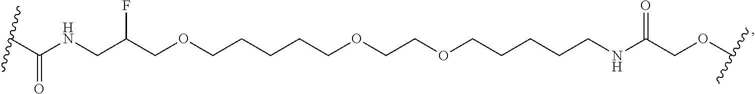 [SMILES: *OCC(=O)NCCCCCOCCOCCCCCOCC(F)CNC(*)=O] | (139) |
| 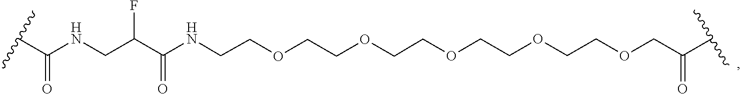 [SMILES: *C(=O)COCCOCCOCCOCCOCCNC(=O)C(F)CNC(*)=O] | (140) |
| 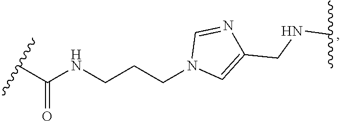 [SMILES: *NCc1cn(CCCNC(*)=O)cn1] | (141) |
| 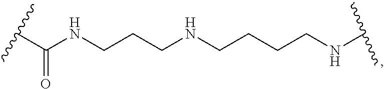 [SMILES: *NCCCCNCCCNC(*)=O] | (142) |
| 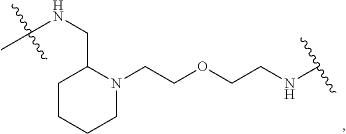 [SMILES: *NCCOCCN1CCCCC1CN*] | (143) |
| 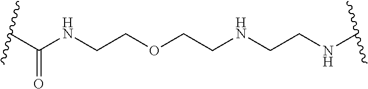 [SMILES: *NCCNCCOCCNC(*)=O] | (144) |
| 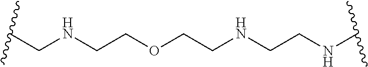 [SMILES: *CNCCOCCNCCN*] | (145) |
| 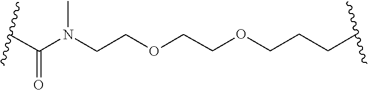 [SMILES: *CCCOCCOCCN(C)C(*)=O] | (146) |
| 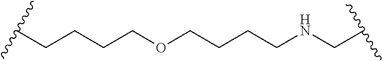 [SMILES: *CCCCOCCCCNC*] | (147) |
| 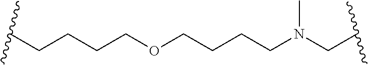 [SMILES: *CCCCOCCCCN(C)C*] | (148) |
| 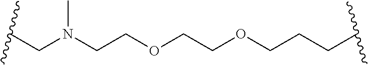 [SMILES: *CCCOCCOCCN(C)C*] | (149) |
| 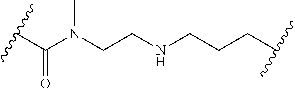 [SMILES: *CCCNCCN(C)C(*)=O] | (150) |
| 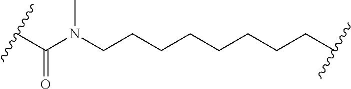 [SMILES: *CCCCCCCCN(C)C(*)=O] | (151) |
| 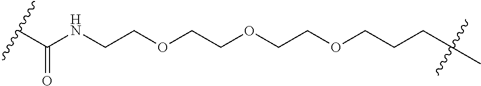 [SMILES: *CCCOCCOCCOCCNC(*)=O] | (152) |
| 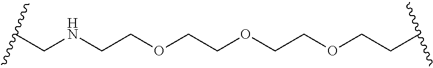 [SMILES: *CCOCCOCCOCCNC*] | (153) |
| 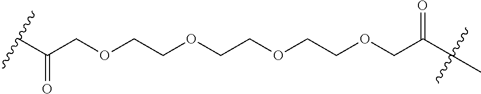 [SMILES: *C(=O)COCCOCCOCCOCC(*)=O] | (154) |
| 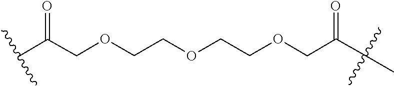 [SMILES: *C(=O)COCCOCCOCC(*)=O] | (155) |
| 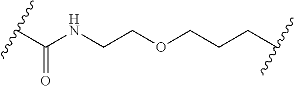 [SMILES: *CCCOCCNC(*)=O] | (156) |
| 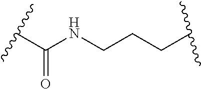 [SMILES: *CCCNC(*)=O] | (157) |
| 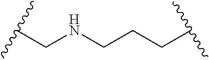 [SMILES: *CCCNC*] | (158) |
| 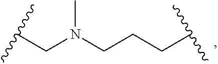 [SMILES: *CCCN(C)C*] | (159) |
| 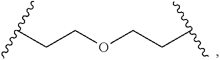 [SMILES: *CCOCC*] | (160) |
| 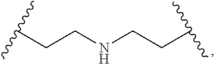 [SMILES: *CCNCC*] | (161) |
| 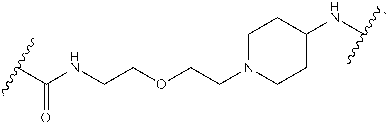 [SMILES: *NC1CCN(CCOCCNC(*)=O)CC1] | (162) |
| 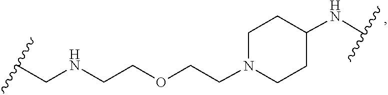 [SMILES: *CNCCOCCN1CCC(N*)CC1] | (163) |
| 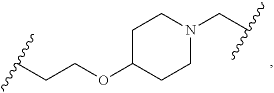 [SMILES: *CCOC1CCN(C*)CC1] | (164) |
| 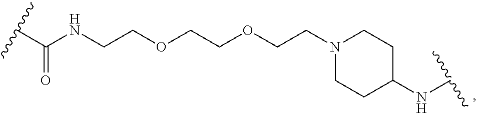 [SMILES: *NC1CCN(CCOCCOCCNC(*)=O)CC1] | (165) |
| 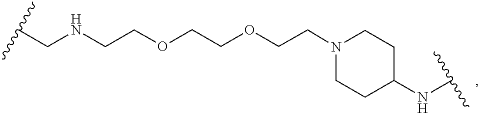 [SMILES: *CNCCOCCOCCN1CCC(N*)CC1] | (166) |
| 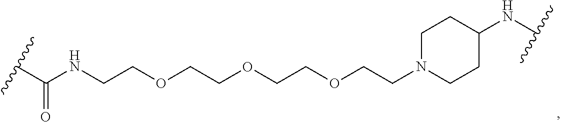 [SMILES: *NC1CCN(CCOCCOCCOCCNC(*)=O)CC1] | (167) |
| 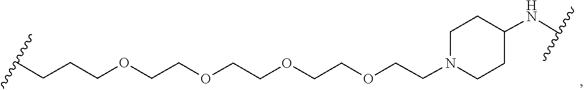 [SMILES: *CCCOCCOCCOCCOCCN1CCC(N*)CC1] | (168) |
| 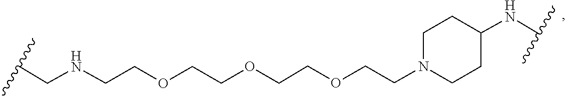 [SMILES: *CNCCOCCOCCOCCN1CCC(N*)CC1] | (169) |
| 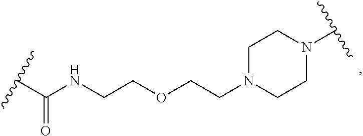 [SMILES: *C(=O)NCCOCCN1CCN(*)CC1] | (170) |
| 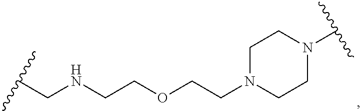 [SMILES: *CNCCOCCN1CCN(*)CC1] | (171) |
| 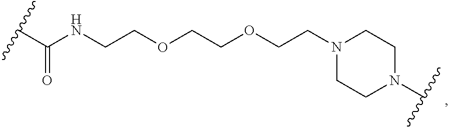 [SMILES: *C(=O)NCCOCCOCCN1CCN(*)CC1] | (172) |
| 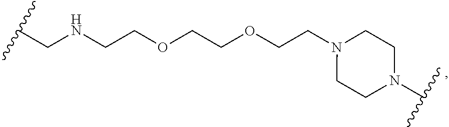 [SMILES: *CNCCOCCOCCN1CCN(*)CC1] | (173) |
| 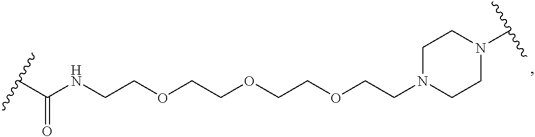 | (174) |
| 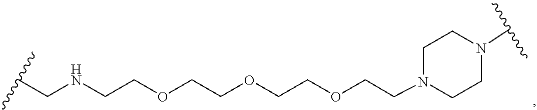 | (175) |
| 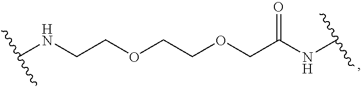 | (176) |
| 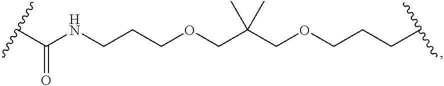 | (177) |
| 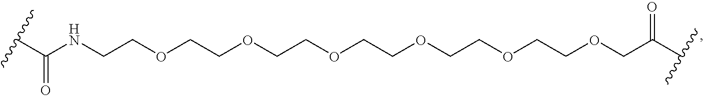 | (178) |
| 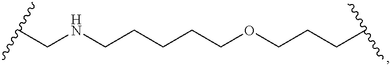 | (179) |
| 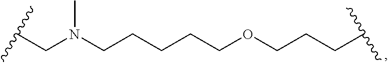 | (180) |
| 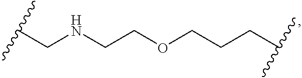 | (181) |
| 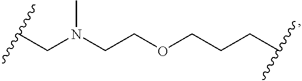 | (182) |
| 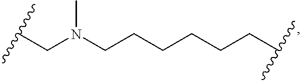 | (183) |
| 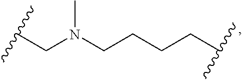 | (184) |
| 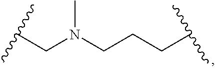 | (185) |
| 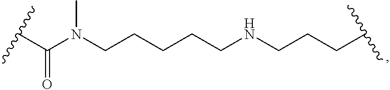 | (186) |
| 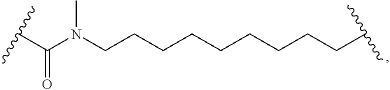 | (187) |
| 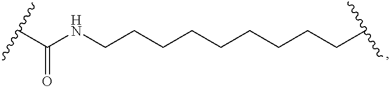 | (188) |
| 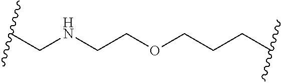 [SMILES: *CCCOCCNC*] | (189) |
| 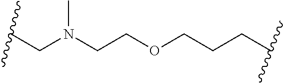 [SMILES: *CCCOCCN(C)C*] | (190) |
| 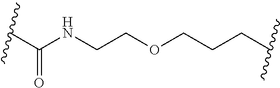 [SMILES: *CCCOCCNC(*)=O] | (191) |
| 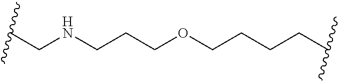 [SMILES: *CCCCOCCCNC*] | (192) |
| 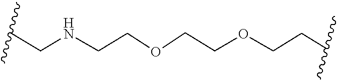 [SMILES: *CCOCCOCCNC*] | (193) |
| 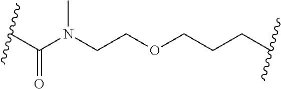 [SMILES: *CCCOCCN(C)C(*)=O] | (194) |
| 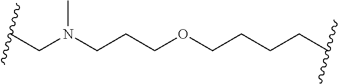 [SMILES: *CCCCOCCCN(C)C*] | (195) |
| 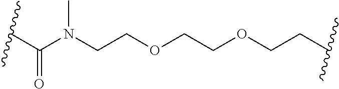 [SMILES: *CCOCCOCCN(C)C(*)=O] | (196) |
| 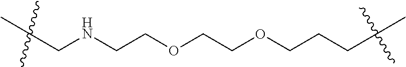 [SMILES: *CCCOCCOCCNC*] | (197) |
| 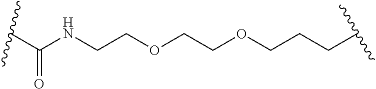 [SMILES: *CCCOCCOCCNC(*)=O] | (198) |
| 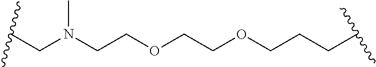 [SMILES: *CCCOCCOCCN(C)C*] | (199) |
| 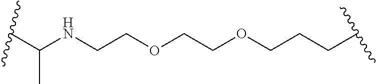 [SMILES: *CCCOCCOCCNC(*)C] | (200) |
| 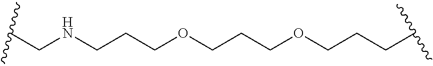 [SMILES: *CCCOCCCOCCCNC*] | (201) |
| 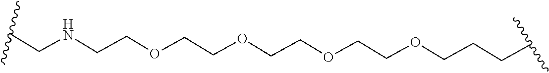 [SMILES: *CCCOCCOCCOCCOCCNC*] | (202) |
| 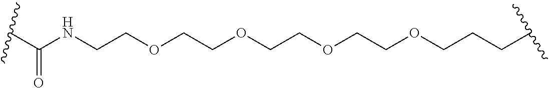 [SMILES: *CCCOCCOCCOCCOCCNC(*)=O] | (203) |
| 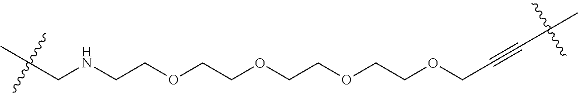 [SMILES: *C#CCOCCOCCOCCOCCNC*] | (204) |
| 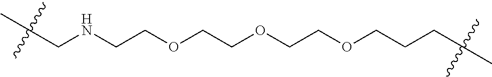 [SMILES: *CCCOCCOCCOCCNC*] | (205) |
| 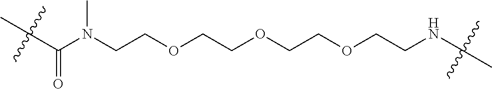 [SMILES: *NCCOCCOCCOCCN(C)C(*)=O] | (206) |
| 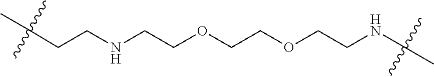 [SMILES: *CCNCCOCCOCCN*] | (207) |
| 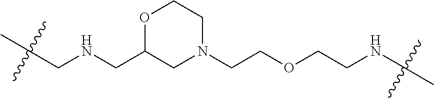 [SMILES: *CNCC1CN(CCOCCN*)CCO1] | (208) |
| 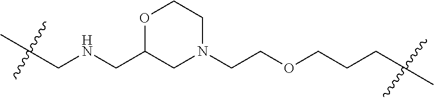 [SMILES: *CCCOCCN1CCOC(CNC*)C1] | (209) |
| 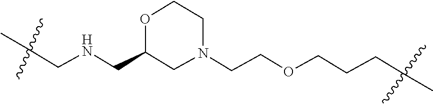 [SMILES: *CCCOCCN1CCO[C@H](CNC*)C1] | (210) |
| 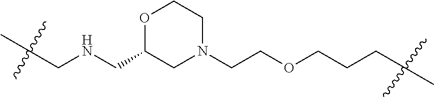 [SMILES: *CCCOCCN1CCO[C@@H](CNC*)C1] | (211) |
| 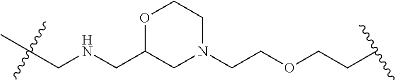 [SMILES: *CCOCCN1CCOC(CNC*)C1] | (212) |
| 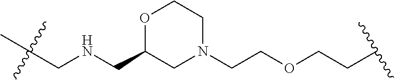 [SMILES: *CCOCCN1CCO[C@H](CNC*)C1] | (213) |
| 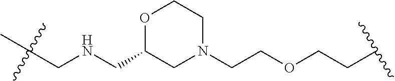 [SMILES: *CCOCCN1CCO[C@@H](CNC*)C1] | (214) |
| 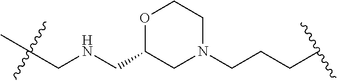 [SMILES: *CCCN1CCO[C@@H](CNC*)C1] | (215) |
| 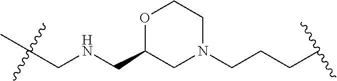 [SMILES: *CCCN1CCO[C@H](CNC*)C1] | (216) |
| 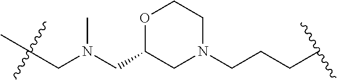 [SMILES: *CCCN1CCO[C@@H](CN(C)C*)C1] | (217) |
| 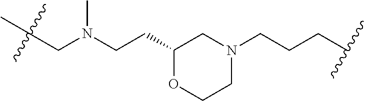 [SMILES: *CCCN1CCO[C@H](CCN(C)C*)C1] | (218) |
| 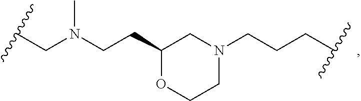 [SMILES: *CCCN1CCO[C@@H](CCN(C)C*)C1] | (219) |
| 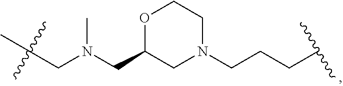 [SMILES: *CCCN1CCO[C@H](CN(C)C*)C1] | (220) |
| 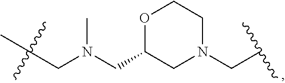 [SMILES: *CN(C)C[C@H]1CN(C*)CCO1] | (221) |
| 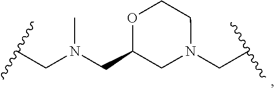 [SMILES: *CN(C)C[C@@H]1CN(C*)CCO1] | (222) |
| 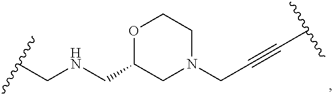 [SMILES: *C#CCN1CCO[C@@H](CNC*)C1] | (223) |
| 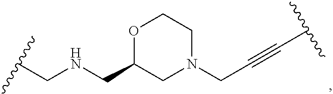 [SMILES: *C#CCN1CCO[C@H](CNC*)C1] | (224) |
| 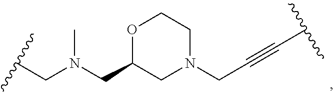 [SMILES: *C#CCN1CCO[C@H](CN(C)C*)C1] | (225) |
| 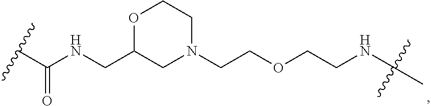 [SMILES: *NCCOCCN1CCOC(CNC(*)=O)C1] | (226) |
| 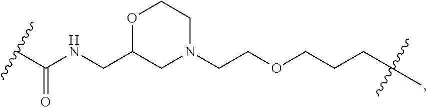 [SMILES: *CCCOCCN1CCOC(CNC(*)=O)C1] | (227) |
| 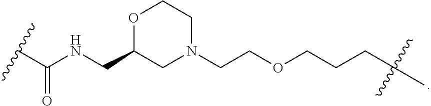 [SMILES: *CCCOCCN1CCO[C@H](CNC(*)=O)C1] | (228) |
| 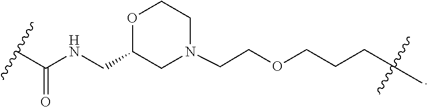 [SMILES: *CCCOCCN1CCO[C@@H](CNC(*)=O)C1] | (229) |
| 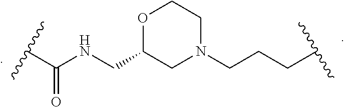 [SMILES: *CCCN1CCO[C@@H](CNC(*)=O)C1] | (230) |
| 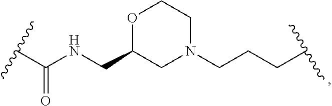 [SMILES: *CCCN1CCO[C@H](CNC(*)=O)C1] | (231) |
| 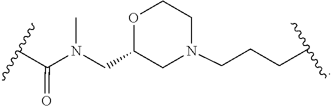 [SMILES: *CCCN1CCO[C@@H](CN(C)C(*)=O)C1] | (232) |
| 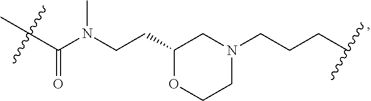 [SMILES: *CCCN1CCO[C@H](CCN(C)C(*)=O)C1] | (233) |
| 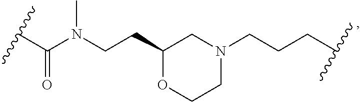 [SMILES: *CCCN1CCO[C@@H](CCN(C)C(*)=O)C1] | (234) |
| 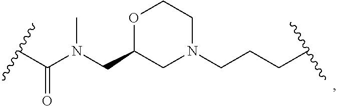 [SMILES: *CCCN1CCO[C@H](CN(C)C(*)=O)C1] | (235) |
| 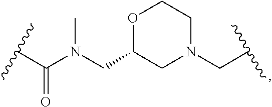 [SMILES: *CN1CCO[C@@H](CN(C)C(*)=O)C1] | (236) |
| 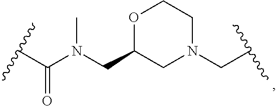 [SMILES: *CN1CCO[C@H](CN(C)C(*)=O)C1] | (237) |
| 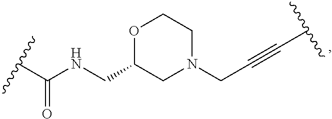 [SMILES: *C#CCN1CCO[C@@H](CNC(*)=O)C1] | (238) |
| 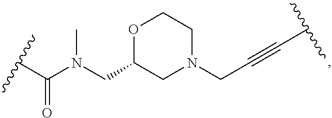 [SMILES: *C#CCN1CCO[C@@H](CN(C)C(*)=O)C1] | (239) |
| 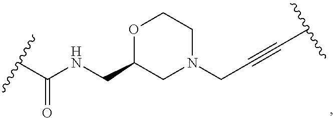 [SMILES: *C#CCN1CCO[C@H](CNC(*)=O)C1] | (240) |
| 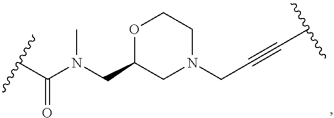 [SMILES: *C#CCN1CCO[C@H](CN(C)C(*)=O)C1] | (241) |
| 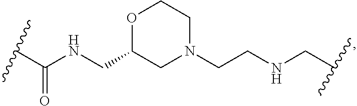 [SMILES: *CNCCN1CCO[C@@H](CNC(*)=O)C1] | (242) |
| 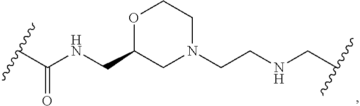 [SMILES: *CNCCN1CCO[C@H](CNC(*)=O)C1] | (243) |
| 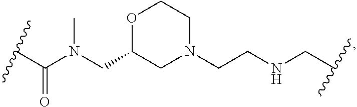 [SMILES: *CNCCN1CCO[C@@H](CN(C)C(*)=O)C1] | (244) |
| 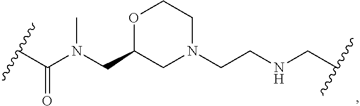 [SMILES: *CNCCN1CCO[C@H](CN(C)C(*)=O)C1] | (245) |
| 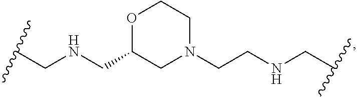 [SMILES: *CNCCN1CCO[C@@H](CNC*)C1] | (246) |
| 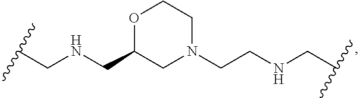 [SMILES: *CNCCN1CCO[C@H](CNC*)C1] | (247) |
| 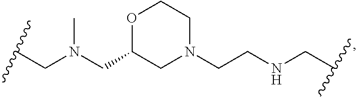 [SMILES: *CNCCN1CCO[C@@H](CN(C)C*)C1] | (248) |
| 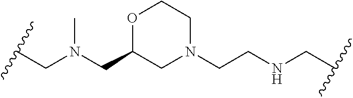 [SMILES: *CNCCN1CCO[C@H](CN(C)C*)C1] | (249) |
| 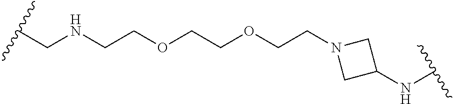 [SMILES: *CNCCOCCOCCN1CC(N*)C1] | (250) |
| 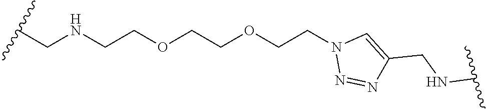 [SMILES: *CNCCOCCOCCn1cc(CN*)nn1] | (251) |
| 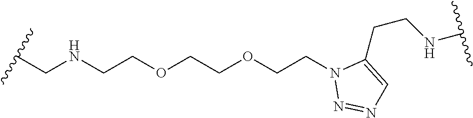 [SMILES: *CNCCOCCOCCn1nncc1CCN*] | (252) |
| 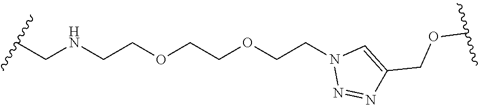 [SMILES: *CNCCOCCOCCn1cc(CO*)nn1] | (253) |
| 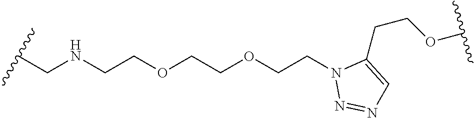 [SMILES: *CNCCOCCOCCn1nncc1CCO*] | (254) |
| 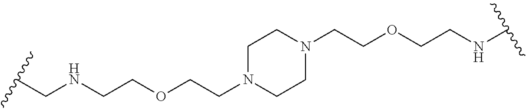 [SMILES: *CNCCOCCN1CCN(CCOCCN*)CC1] | (255) |
| 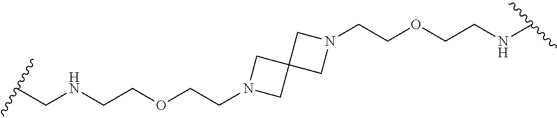 [SMILES: *CNCCOCCN1CC2(CN(CCOCCN*)C2)C1] | (256) |
| 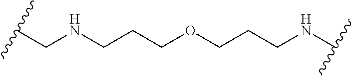 [SMILES: *CNCCCOCCCN*] | (257) |
| 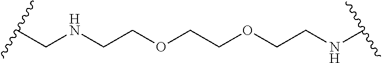 [SMILES: *CNCCOCCOCCN*] | (258) |
| 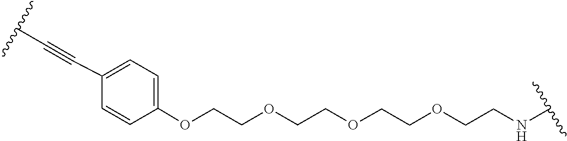 [SMILES: *C#Cc1ccc(OCCOCCOCCOCCN*)cc1] | (259) |
| 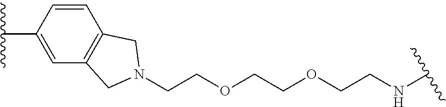 [SMILES: *NCCOCCOCCN1Cc2ccc(*)cc2C1] | (260) |
| 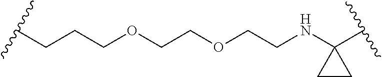 [SMILES: *CCCOCCOCCNC1(*)CC1] | (261) |
| 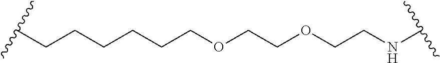 [SMILES: *CCCCCCOCCOCCN*] | (262) |
| 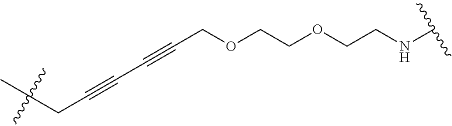 [SMILES: *CC#CC#CCOCCOCCN*] | (263) |
| 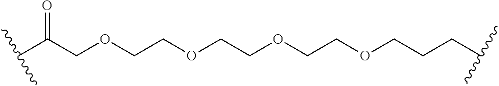 [SMILES: *CCCOCCOCCOCCOCC(*)=O] | (264) |
| 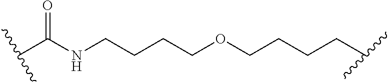 [SMILES: *CCCCOCCCCNC(*)=O] | (265) |
| 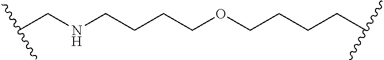 [SMILES: *CCCCOCCCCNC*] | (266) |
| 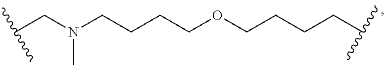 [SMILES: *CCCCOCCCCN(C)C*] | (267) |
| 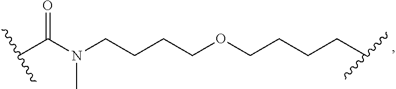 [SMILES: *CCCCOCCCCN(C)C(*)=O] | (268) |
| 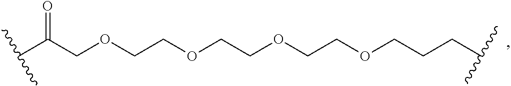 [SMILES: *CCCOCCOCCOCCOCC(*)=O] | (269) |
| 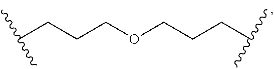 [SMILES: *CCCOCCC*] | (270) |
| 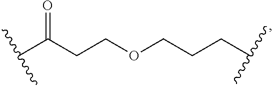 [SMILES: *CCCOCCC(*)=O] | (271) |
| 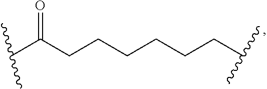 [SMILES: *CCCCCCC(*)=O] | (272) |
| 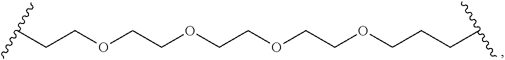 [SMILES: *CCCOCCOCCOCCOCC*] | (273) |
| 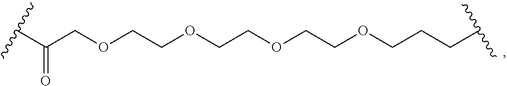 [SMILES: *CCCOCCOCCOCCOCC(*)=O] | (274) |
| 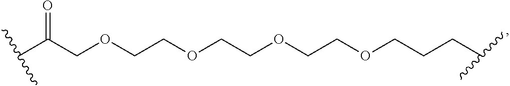 [SMILES: *CCCOCCOCCOCCOCC(*)=O] | (275) |
| 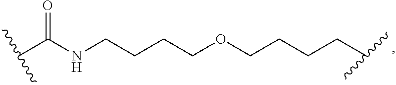 [SMILES: *CCCCOCCCCNC(*)=O] | (276) |
| 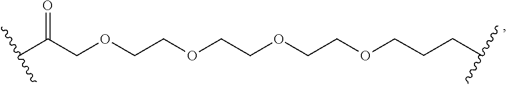 [SMILES: *CCCOCCOCCOCCOCC(*)=O] | (277) |
| 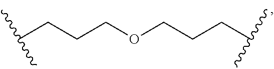 [SMILES: *CCCOCCC*] | (278) |
| 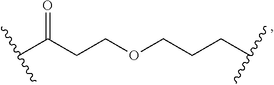 [SMILES: *CCCOCCC(*)=O] | (279) |
| 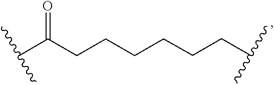 [SMILES: *CCCCCCC(*)=O] | (280) |
| 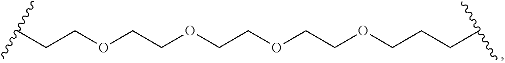 [SMILES: *CCCOCCOCCOCCOCC*] | (281) |
| 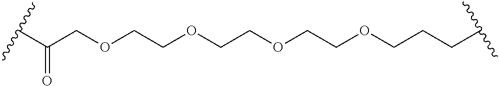 [SMILES: *CCCOCCOCCOCCOCC(*)=O] | (282) |
| 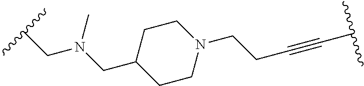 [SMILES: *C#CCCN1CCC(CN(C)C*)CC1] | (283) |
| 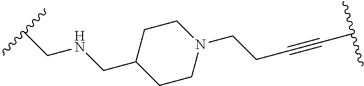 [SMILES: *C#CCCN1CCC(CNC*)CC1] | (284) |
| 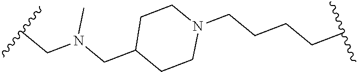 [SMILES: *CCCCN1CCC(CN(C)C*)CC1] | (285) |
| 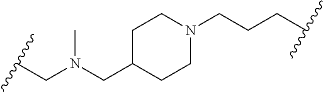 [SMILES: *CCCN1CCC(CN(C)C*)CC1] | (286) |
| 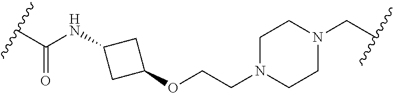 [SMILES: *CN1CCN(CCO[C@H]2C[C@H](NC(*)=O)C2)CC1] | (287) |
| 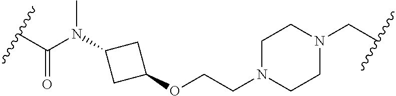 [SMILES: *CN1CCN(CCO[C@H]2C[C@H](N(C)C(*)=O)C2)CC1] | (288) |
| 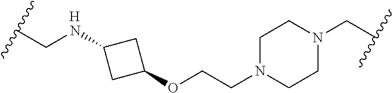 | (289) |
| 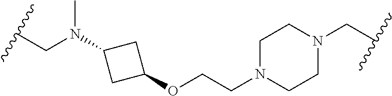 [SMILES: *CN1CCN(CCO[C@H]2C[C@H](N(C)C*)C2)CC1] | (290) |
| 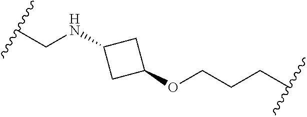 [SMILES: *CCCO[C@H]1C[C@H](NC*)C1] | (291) |
| 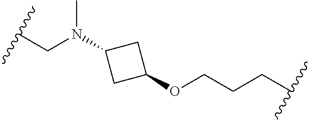 [SMILES: *CCCO[C@H]1C[C@H](N(C)C*)C1] | (292) |
| 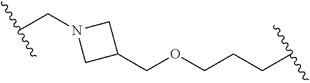 [SMILES: *CCCOCC1CN(C*)C1] | (293) |
| 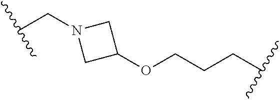 [SMILES: *CCCOC1CN(C*)C1] | (294) |
| 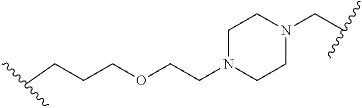 [SMILES: *CCCOCCN1CCN(C*)CC1] | (295) |
| 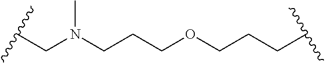 [SMILES: *CCCOCCCN(C)C*] | (296) |
| 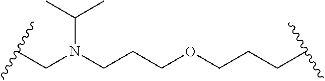 [SMILES: *CCCOCCCN(C*)C(C)C] | (297) |
| 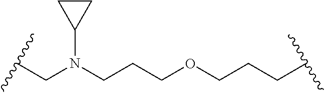 [SMILES: *CCCOCCCN(C*)C1CC1] | (298) |
| 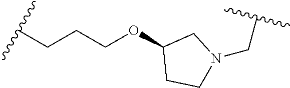 [SMILES: *CCCO[C@@H]1CCN(C*)C1] | (299) |
| 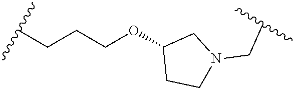 [SMILES: *CCCO[C@H]1CCN(C*)C1] | (300) |
| 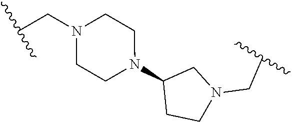 [SMILES: *CN1CCN([C@@H]2CCN(C*)C2)CC1] | (301) |
| 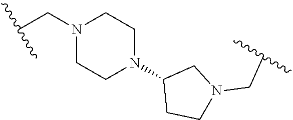 [SMILES: *CN1CCN([C@H]2CCN(C*)C2)CC1] | (302) |
| 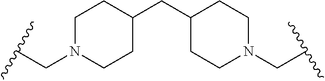 [SMILES: *CN1CCC(CC2CCN(C*)CC2)CC1] | (303) |
| 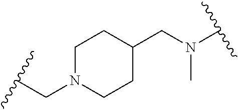 [SMILES: *CN1CCC(CN(*)C)CC1] | (304) |
| 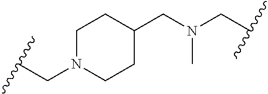 [SMILES: *CN(C)CC1CCN(C*)CC1] | (305) |
| 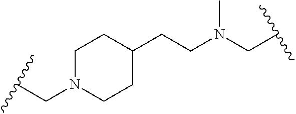 [SMILES: *CN(C)CCC1CCN(C*)CC1] | (306) |
| 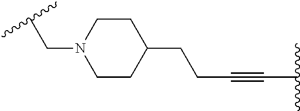 [SMILES: *C#CCCC1CCN(C*)CC1] | (307) |
| 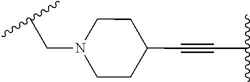 [SMILES: *C#CC1CCN(C*)CC1] | (308) |
| 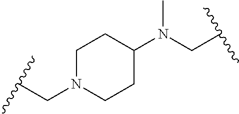 [SMILES: *CN1CCC(N(C)C*)CC1] | (309) |
| 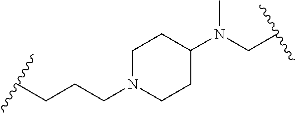 [SMILES: *CCCN1CCC(N(C)C*)CC1] | (310) |
| 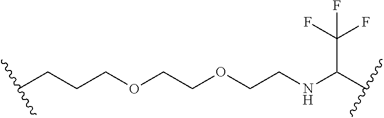 [SMILES: *CCCOCCOCCNC(*)C(F)(F)F] | (311) |
| 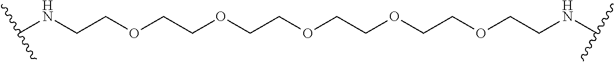 [SMILES: *NCCOCCOCCOCCOCCOCCN*] | (312) |
| 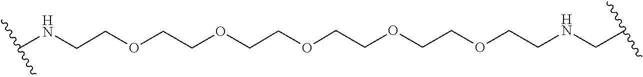 [SMILES: *CNCCOCCOCCOCCOCCOCCN*] | (313) |
| 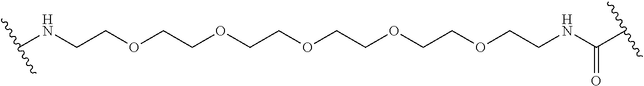 [SMILES: *NCCOCCOCCOCCOCCOCCNC(*)=O] | (314) |
| 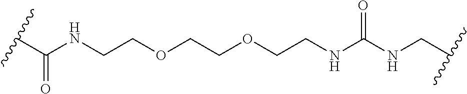 [SMILES: *CNC(=O)NCCOCCOCCNC(*)=O] | (315) |
| 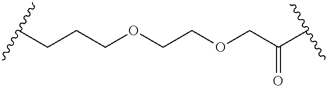 [SMILES: *CCCOCCOCC(*)=O] | (316) |
| 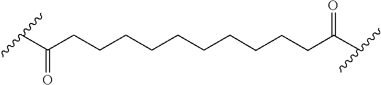 [SMILES: *C(=O)CCCCCCCCCCC(*)=O] | (317) |
| 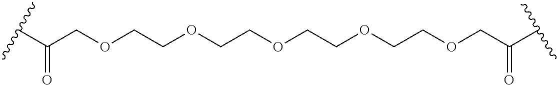 [SMILES: *C(=O)COCCOCCOCCOCCOCC(*)=O] | (318) |
| 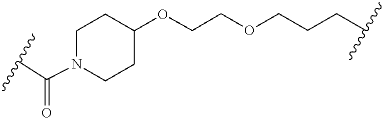 [SMILES: *CCCOCCOC1CCN(C(*)=O)CC1] | (319) |
| 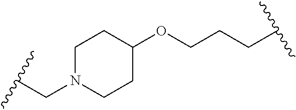 [SMILES: *CCCOC1CCN(C*)CC1] | (320) |
| 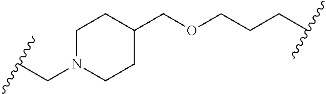 [SMILES: *CCCOCC1CCN(C*)CC1] | (321) |
| 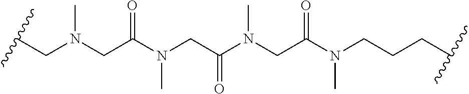 [SMILES: *CCCN(C)C(=O)CN(C)C(=O)CN(C)C(=O)CN(C)C*] | (322) |
| 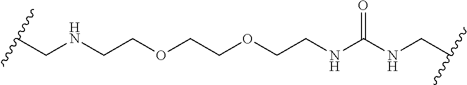 [SMILES: *CNCCOCCOCCNC(=O)NC*] | (323) |
| 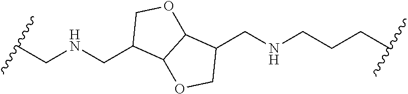 [SMILES: *CCCNCC1COC2C(CNC*)COC12] | (324) |
| 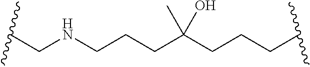 [SMILES: *CCCC(C)(O)CCCNC*] | (325) |
| 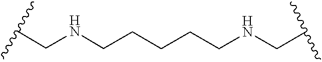 [SMILES: *CNCCCCCNC*] | (326) |
| 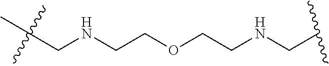 [SMILES: *CNCCOCCNC*] | (327) |
| 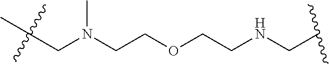 [SMILES: *CNCCOCCN(C)C*] | (328) |
| 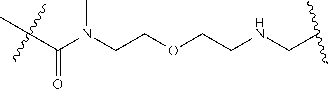 [SMILES: *CNCCOCCN(C)C(*)=O] | (329) |
| 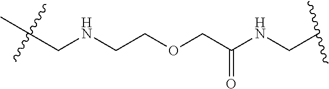 [SMILES: *CNCCOCC(=O)NC*] | (330) |
| 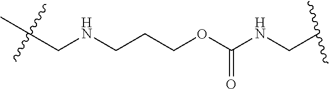 [SMILES: *CNCCCOC(=O)NC*] | (331) |
| 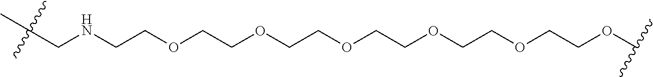 [SMILES: *CNCCOCCOCCOCCOCCOCCO*] | (332) |
| 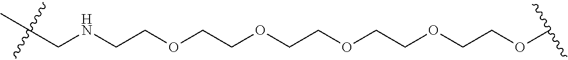 [SMILES: *CNCCOCCOCCOCCOCCO*] | (333) |
| 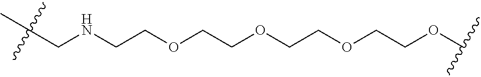 [SMILES: *CNCCOCCOCCOCCO*] | (334) |
| 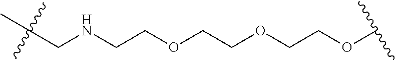 [SMILES: *CNCCOCCOCCO*] | (335) |
| 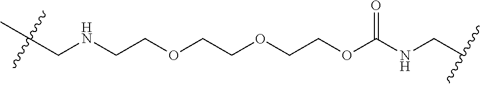 [SMILES: *CNCCOCCOCCOC(=O)NC*] | (336) |
| 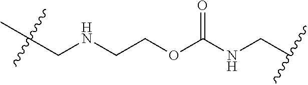 [SMILES: *CNCCOC(=O)NC*] | (337) |
| 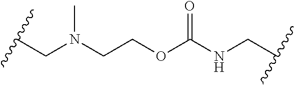 [SMILES: *CNC(=O)OCCN(C)C*] | (338) |
| 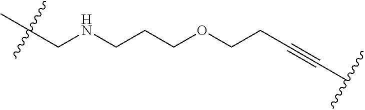 [SMILES: *C#CCCOCCCNC*] | (339) |
| 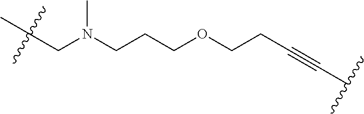 [SMILES: *C#CCCOCCCN(C)C*] | (340) |
| 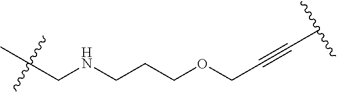 [SMILES: *C#CCOCCCNC*] | (341) |
| 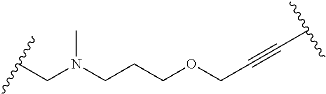 [SMILES: *C#CCOCCCN(C)C*] | (342) |
| 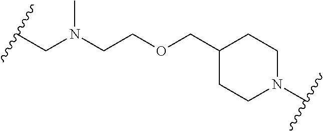 [SMILES: *CN(C)CCOCC1CCN(*)CC1] | (343) |
| 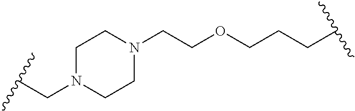 [SMILES: *CCCOCCN1CCN(C*)CC1] | (344) |
| 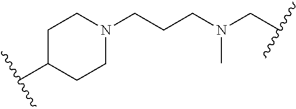 [SMILES: *CN(C)CCCN1CCC(*)CC1] | (345) |
| 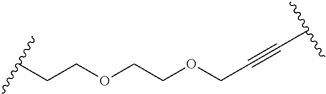 [SMILES: *C#CCOCCOCC*] | (346) |
| 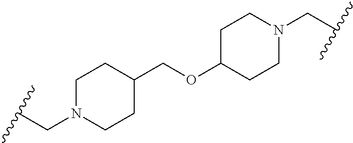 [SMILES: *CN1CCC(COC2CCN(C*)CC2)CC1] | (347) |
| 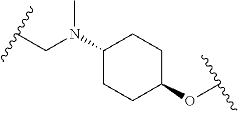 [SMILES: *CN(C)[C@H]1CC[C@H](O*)CC1] | (348) |
| 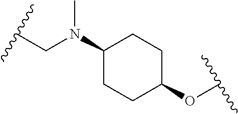 [SMILES: *CN(C)[C@H]1CC[C@@H](O*)CC1] | (349) |
| 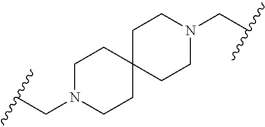 [SMILES: *CN1CCC2(CC1)CCN(C*)CC2] | (350) |
| 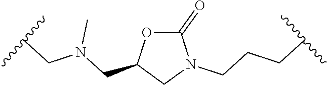 [SMILES: *CCCN1C[C@@H](CN(C)C*)OC1=O] | (351) |
| 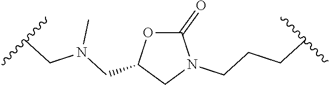 [SMILES: *CCCN1C[C@H](CN(C)C*)OC1=O] | (352) |
| 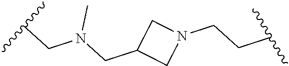 [SMILES: *CCN1CC(CN(C)C*)C1] | (353) |
| 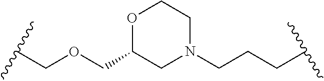 [SMILES: *CCCN1CCO[C@@H](COC*)C1] | (354) |
| 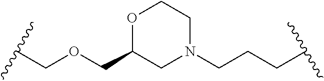 [SMILES: *CCCN1CCO[C@H](COC*)C1] | (355) |
| 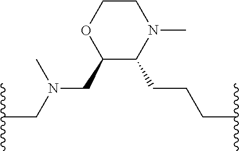 [SMILES: *CCC[C@@H]1[C@@H](CN(C)C*)OCCN1C] | (356) |
| 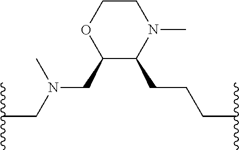 [SMILES: *CCC[C@H]1[C@@H](CN(C)C*)OCCN1C] | (357) |
| 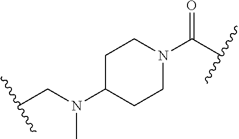 [SMILES: *CN(C)C1CCN(C(*)=O)CC1] | (358) |
| 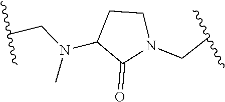 [SMILES: *CN1CCC(N(C)C*)C1=O] | (359) |
| 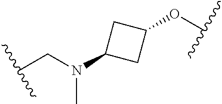 [SMILES: *CN(C)[C@H]1C[C@H](O*)C1] | (360) |
| 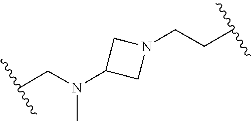 [SMILES: *CCN1CC(N(C)C*)C1] | (361) |
| 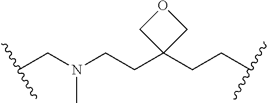 [SMILES: *CCC1(CCN(C)C*)COC1] | (362) |
| 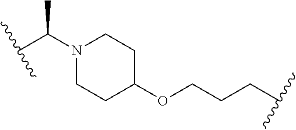 [SMILES: *CCCOC1CCN([C@@H](*)C)CC1] | (363) |
| 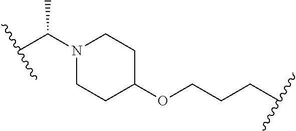 [SMILES: *CCCOC1CCN([C@H](*)C)CC1] | (364) |
| 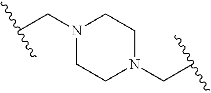 [SMILES: *CN1CCN(C*)CC1] | (365) |
| 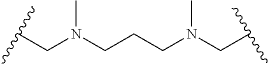 [SMILES: *CN(C)CCCN(C)C*] | (366) |
| 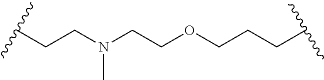 [SMILES: *CCCOCCN(C)CC*] | (367) |
| 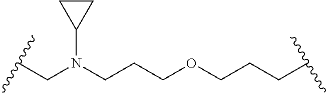 [SMILES: *CCCOCCCN(C*)C1CC1] | (368) |
| 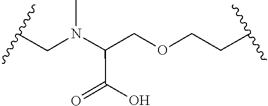 [SMILES: *CCOCC(C(=O)O)N(C)C*] | (369) |
| 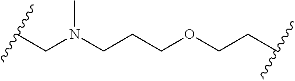 [SMILES: *CCOCCCN(C)C*] | (370) |
| 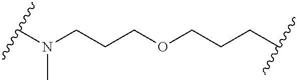 [SMILES: *CCCOCCCN(*)C] | (371) |
| 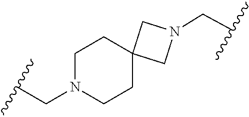 [SMILES: *CN1CCC2(CC1)CN(C*)C2] | (372) |
| 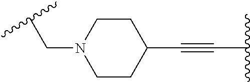 [SMILES: *C#CC1CCN(C*)CC1] | (373) |
| 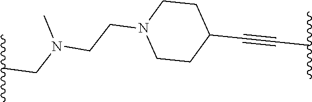 [SMILES: *C#CC1CCN(CCN(C)C*)CC1] | (374) |
| 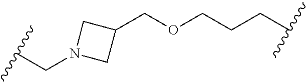 [SMILES: *CCCOCC1CN(C*)C1] | (375) |
| 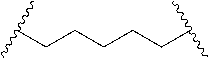 [SMILES: *CCCCC*] | (376) |
| 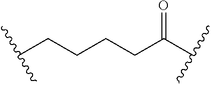 [SMILES: *CCCCC(*)=O] | (377) |
| 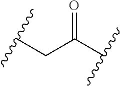 [SMILES: *CC(*)=O] | (378) |
| 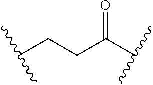 [SMILES: *CCC(*)=O] | (379) |
| 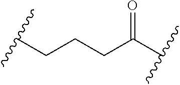 [SMILES: *CCCC(*)=O] | (380) |
| 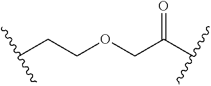 [SMILES: *CCOCC(*)=O] | (381) |
| 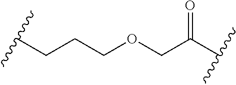 [SMILES: *CCCOCC(*)=O] | (382) |
| 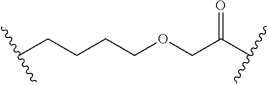 [SMILES: *CCCCOCC(*)=O] | (383) |
| 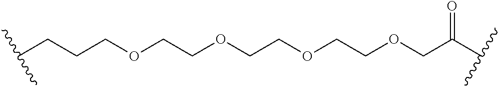 [SMILES: *CCCOCCOCCOCCOCC(*)=O] | (384) |
| 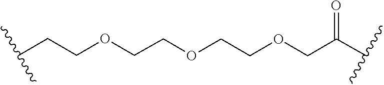 [SMILES: *CCOCCOCCOCC(*)=O] | (385) |
| 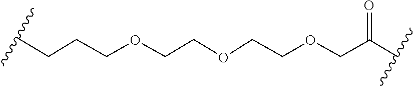 [SMILES: *CCCOCCOCCOCC(*)=O] | (386) |
| 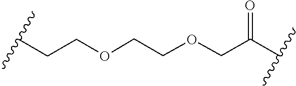 [SMILES: *CCOCCOCC(*)=O] | (387) |
| 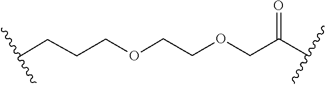 [SMILES: *CCCOCCOCC(*)=O] | (388) |
| 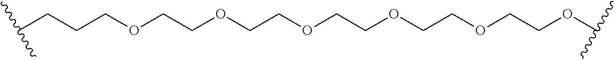 [SMILES: *CCCOCCOCCOCCOCCOCCO*] | (389) |
| 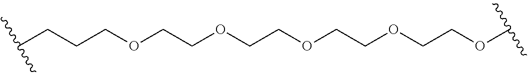 [SMILES: *CCCOCCOCCOCCOCCO*] | (390) |
| 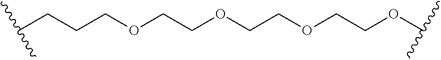 [SMILES: *CCCOCCOCCOCCO*] | (391) |
| 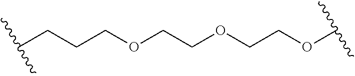 [SMILES: *CCCOCCOCCO*] | (392) |
| 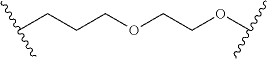 [SMILES: *CCCOCCO*] | (393) |
| 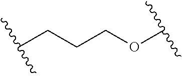 [SMILES: *CCCO*] | (394) |
| 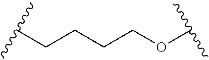 [SMILES: *CCCCO*] | (395) |
| 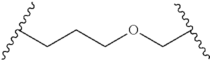 [SMILES: *CCCOC*] | (396) |
| 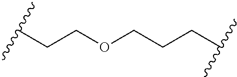 [SMILES: *CCCOCC*] | (397) |
| 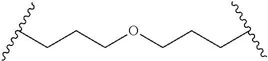 [SMILES: *CCCOCCC*] | (398) |
| 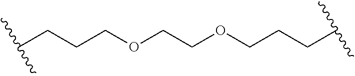 [SMILES: *CCCOCCOCCC*] | (399) |
| 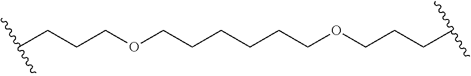 [SMILES: *CCCOCCCCCCOCCC*] | (400) |
| 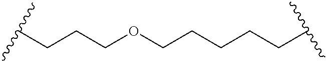 [SMILES: *CCCCCOCCC*] | (401) |
| 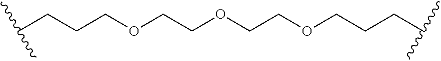 [SMILES: *CCCOCCOCCOCCC*] | (402) |
| 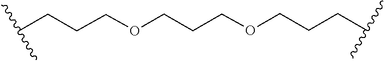 [SMILES: *CCCOCCCOCCC*] | (403) |
| 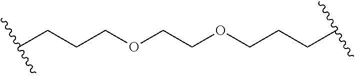 [SMILES: *CCCOCCOCCC*] | (404) |
| 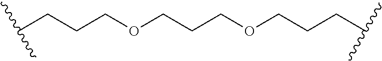 [SMILES: *CCCOCCCOCCC*] | (405) |
| 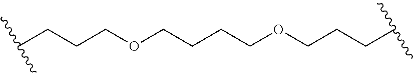 [SMILES: *CCCOCCCCOCCC*] | (406) |
| 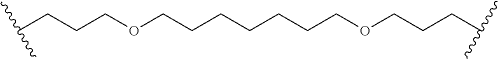 [SMILES: *CCCOCCCCCCCOCCC*] | (407) |
| 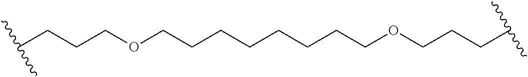 [SMILES: *CCCOCCCCCCCCOCCC*] | (408) |
| 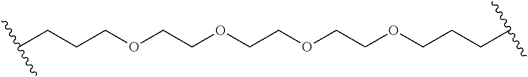 [SMILES: *CCCOCCOCCOCCOCCC*] | (409) |
| 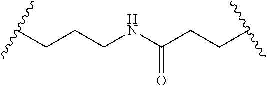 [SMILES: *CCCNC(=O)CC*] | (410) |
| 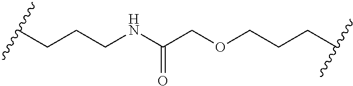 [SMILES: *CCCNC(=O)COCCC*] | (411) |
| 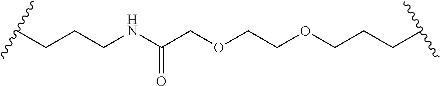 [SMILES: *CCCNC(=O)COCCOCCC*] | (412) |
| 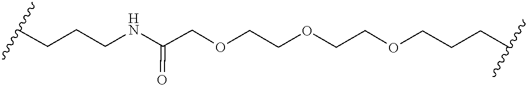 [SMILES: *CCCNC(=O)COCCOCCOCCC*] | (413) |
| 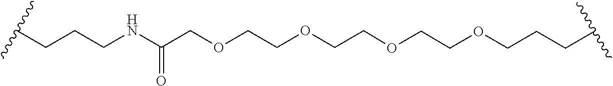 [SMILES: *CCCNC(=O)COCCOCCOCCOCCC*] | (414) |
| 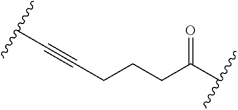 [SMILES: *C#CCCCC(*)=O] | (415) |
| 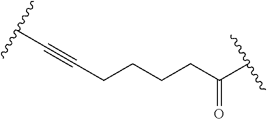 [SMILES: *C#CCCCCC(*)=O] | (416) |
| 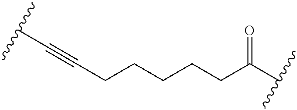 [SMILES: *C#CCCCCCC(*)=O] | (417) |
| 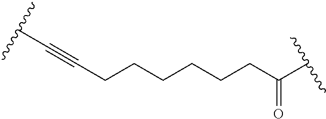 [SMILES: *C#CCCCCCCC(*)=O] | (418) |
| 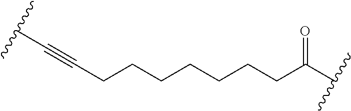 [SMILES: *C#CCCCCCCCC(*)=O] | (419) |
| 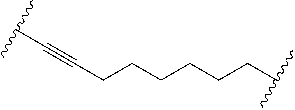 [SMILES: *C#CCCCCCC*] | (420) |
| 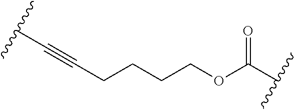 [SMILES: *C#CCCCCOC(*)=O] | (421) |
| 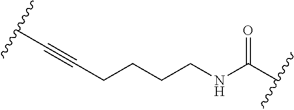 [SMILES: *C#CCCCCNC(*)=O] | (422) |
| 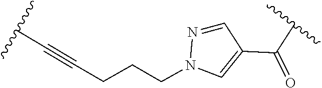 [SMILES: *C#CCCCn1cc(C(*)=O)cn1] | (423) |
| 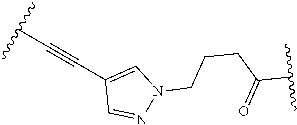 [SMILES: *C#Cc1cnn(CCCC(*)=O)c1] | (424) |
| 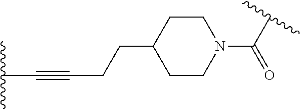 [SMILES: *C#CCCC1CCN(C(*)=O)CC1] | (425) |
| 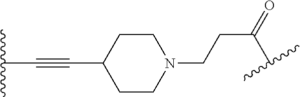 [SMILES: *C#CC1CCN(CCC(*)=O)CC1] | (426) |
| 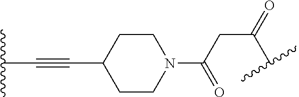 [SMILES: *C#CC1CCN(C(=O)CC(*)=O)CC1] | (427) |
| 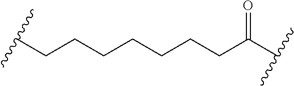 [SMILES: *CCCCCCCC(*)=O] | (428) |
| 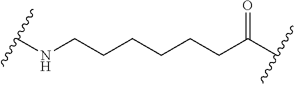 [SMILES: *NCCCCCCC(*)=O] | (429) |
| 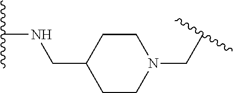 [SMILES: *CN1CCC(CN*)CC1] | (430) |
| 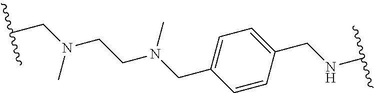 [SMILES: *CN(C)CCN(C)Cc1ccc(CN*)cc1] | (431) |
| 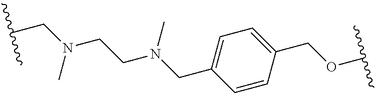 [SMILES: *CN(C)CCN(C)Cc1ccc(CO*)cc1] | (432) |
| 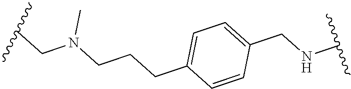 [SMILES: *CN(C)CCCc1ccc(CN*)cc1] | (433) |
| 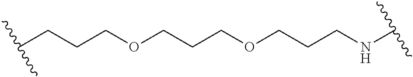 [SMILES: *CCCOCCCOCCCN*] | (434) |
| 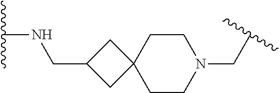 [SMILES: *CN1CCC2(CC1)CC(CN*)C2] | (435) |
| 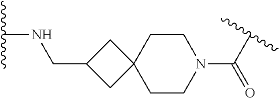 [SMILES: *NCC1CC2(CCN(C(*)=O)CC2)C1] | (436) |
| 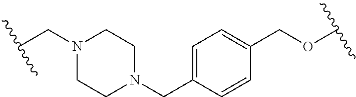 [SMILES: *CN1CCN(Cc2ccc(CO*)cc2)CC1] | (437) |
| 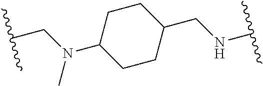 [SMILES: *CN(C)C1CCC(CN*)CC1] | (438) |
| 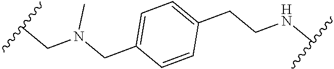 [SMILES: *CN(C)Cc1ccc(CCN*)cc1] | (439) |
| 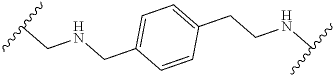 [SMILES: *CNCc1ccc(CCN*)cc1] | (440) |
| 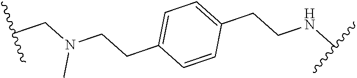 [SMILES: *CN(C)CCc1ccc(CCN*)cc1] | (441) |
| 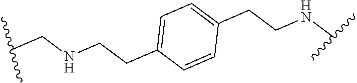 [SMILES: *CNCCc1ccc(CCN*)cc1] | (442) |
| 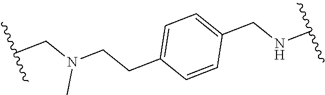 [SMILES: *CN(C)CCc1ccc(CN*)cc1] | (443) |
| 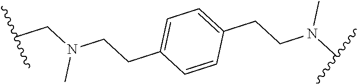 [SMILES: *CN(C)CCc1ccc(CCN(*)C)cc1] | (444) |
| 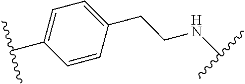 [SMILES: *NCCc1ccc(*)cc1] | (445) |
| 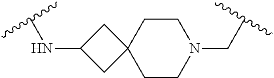 [SMILES: *CN1CCC2(CC1)CC(N*)C2] | (446) |
| 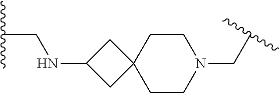 [SMILES: *CNC1CC2(CCN(C*)CC2)C1] | (447) |
| 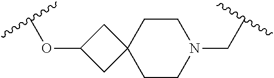 [SMILES: *CN1CCC2(CC1)CC(O*)C2] | (448) |
| 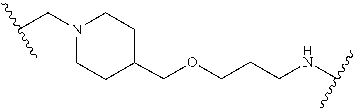 [SMILES: *CN1CCC(COCCCN*)CC1] | (449) |
| 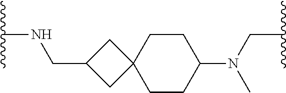 [SMILES: *CN(C)C1CCC2(CC1)CC(CN*)C2] | (450) |
| 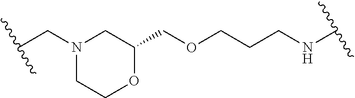 [SMILES: *CN1CCO[C@@H](COCCCN*)C1] | (451) |
| 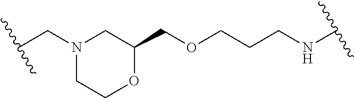 [SMILES: *CN1CCO[C@H](COCCCN*)C1] | (452) |
| 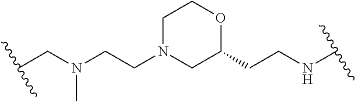 [SMILES: *CN(C)CCN1CCO[C@H](CCN*)C1] | (453) |
| 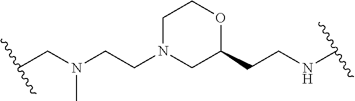 [SMILES: *CN(C)CCN1CCO[C@@H](CCN*)C1] | (454) |
| 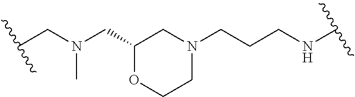 [SMILES: *CN(C)C[C@@H]1CN(CCCN*)CCO1] | (455) |
| 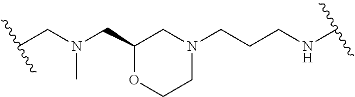 [SMILES: *CN(C)C[C@H]1CN(CCCN*)CCO1] | (456) |
| 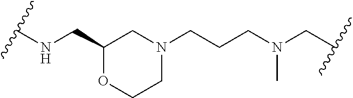 [SMILES: *CN(C)CCCN1CCO[C@@H](CN*)C1] | (457) |
| 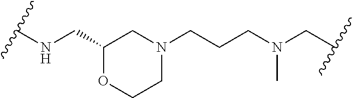 [SMILES: *CN(C)CCCN1CCO[C@H](CN*)C1] | (458) |
| 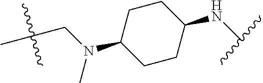 [SMILES: *CN(C)[C@H]1CC[C@@H](N*)CC1] | (459) |
| 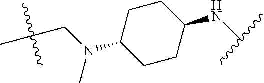 [SMILES: *CN(C)[C@H]1CC[C@H](N*)CC1] | (460) |
| 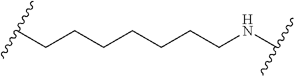 [SMILES: *CCCCCCCN*] | (461) |
| 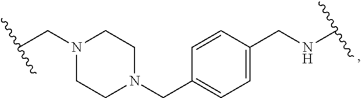 [SMILES: *CN1CCN(Cc2ccc(CN*)cc2)CC1 ; ","] | (462) |
| 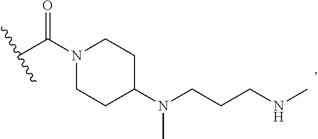 [SMILES: *C(=O)N1CCC(N(C)CCCNC)CC1] | (463) |
| 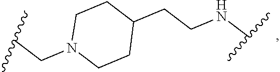 [SMILES: *CN1CCC(CCN*)CC1] | (464) |
| 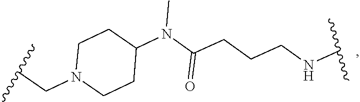 [SMILES: *CN1CCC(N(C)C(=O)CCCN*)CC1] | (465) |
| 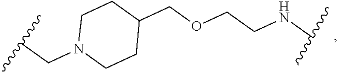 [SMILES: *CN1CCC(COCCN*)CC1] | (466) |
| 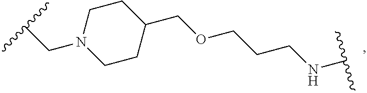 [SMILES: *CN1CCC(COCCCN*)CC1] | (467) |
| 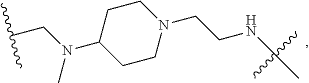 [SMILES: *CN(C)C1CCN(CCN*)CC1] | (468) |
| 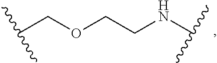 [SMILES: *COCCN*] | (469) |
| 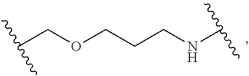 [SMILES: *COCCCN*] | (470) |
| 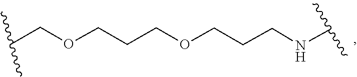 [SMILES: *COCCCOCCCN*] | (471) |
| 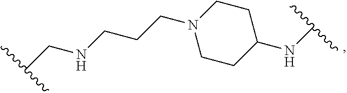 [SMILES: *CNCCCN1CCC(N*)CC1] | (472) |
| 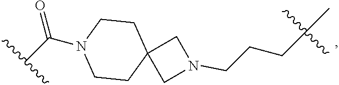 [SMILES: *CCCN1CC2(CCN(C(*)=O)CC2)C1] | (473) |
| 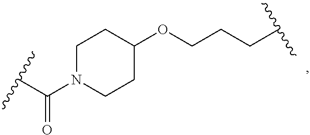 [SMILES: *CCCOC1CCN(C(*)=O)CC1] | (474) |
| 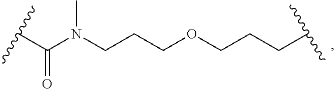 [SMILES: *CCCOCCCN(C)C(*)=O] | (475) |
| 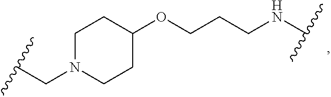 [SMILES: *CN1CCC(OCCCN*)CC1] | (476) |
| 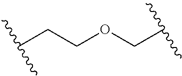 [SMILES: *CCOC*] | (477) |
| 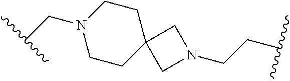 [SMILES: *CCN1CC2(CCN(C*)CC2)C1] | (478) |
| 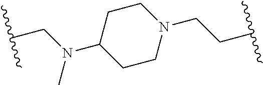 [SMILES: *CCN1CCC(N(C)C*)CC1] | (479) |
| 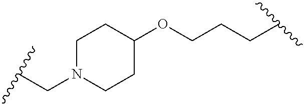 [SMILES: *CCCOC1CCN(C*)CC1] | (480) |
| 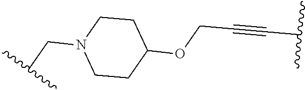 [SMILES: *C#CCOC1CCN(C*)CC1] | (481) |
| 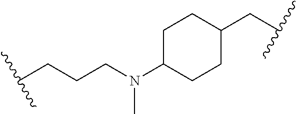 [SMILES: *CCCN(C)C1CCC(C*)CC1] | (482) |
| 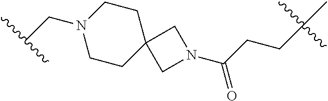 [SMILES: *CCC(=O)N1CC2(CCN(C*)CC2)C1] | (483) |
| 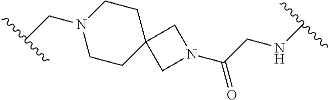 [SMILES: *CN1CCC2(CC1)CN(C(=O)CN*)C2] | (484) |
| 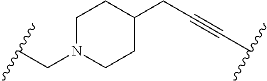 [SMILES: *C#CCC1CCN(C*)CC1] | (485) |
| 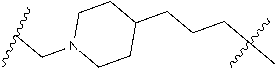 [SMILES: *CCCC1CCN(C*)CC1] | (486) |
| 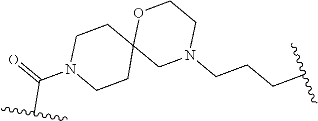 [SMILES: *CCCN1CCOC2(CCN(C(*)=O)CC2)C1] | (487) |
| 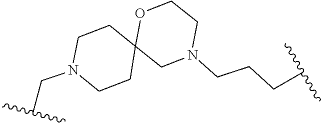 [SMILES: *CCCN1CCOC2(CCN(C*)CC2)C1] | (488) |
| 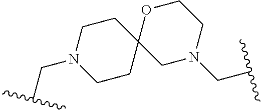 [SMILES: *CN1CCC2(CC1)CN(C*)CCO2] | (489) |
| 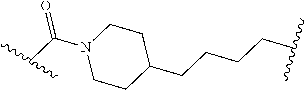 [SMILES: *CCCCC1CCN(C(*)=O)CC1] | (490) |
| 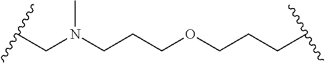 [SMILES: *CCCOCCCN(C)C*] | (491) |
| 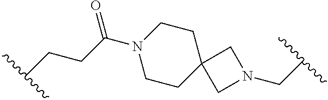 [SMILES: *CCC(=O)N1CCC2(CC1)CN(C*)C2] | (492) |
| 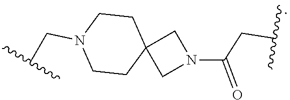 [SMILES: *CC(=O)N1CC2(CCN(C*)CC2)C1] | (493) |
| 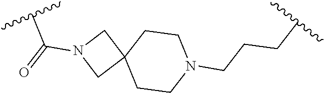 [SMILES: *CCCN1CCC2(CC1)CN(C(*)=O)C2] | (494) |
| 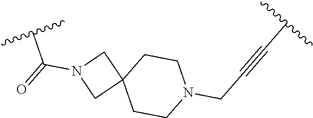 [SMILES: *C#CCN1CCC2(CC1)CN(C(*)=O)C2] | (495) |
| 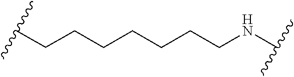 [SMILES: *CCCCCCCN*] | (496) |
| 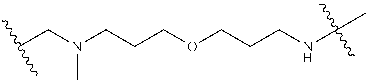 [SMILES: *CN(C)CCCOCCCN*] | (497) |
| 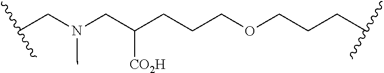 [SMILES: *CCCOCCCC(CN(C)C*)C(=O)O] | (498) |
| 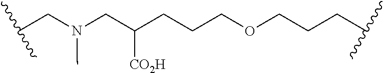 [SMILES: *CCCOCCCC(CN(C)C*)C(=O)O] | (499) |
| 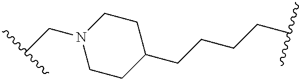 [SMILES: *CCCCC1CCN(C*)CC1] | (500) |
| 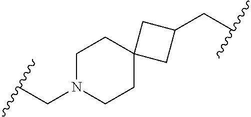 [SMILES: *CC1CC2(CCN(C*)CC2)C1] | (501) |
| 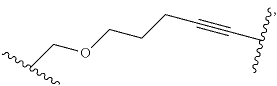 [SMILES: *C#CCCCOC*] | (502) |
| 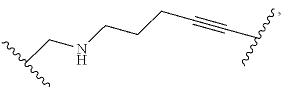 [SMILES: *C#CCCCNC*] | (503) |
| 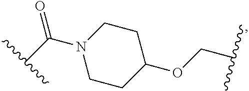 [SMILES: *COC1CCN(C(*)=O)CC1] | (504) |
| 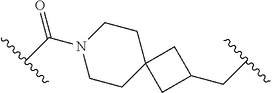 [SMILES: *CC1CC2(CCN(C(*)=O)CC2)C1] | (505) |
| 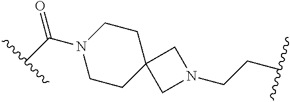 [SMILES: *CCN1CC2(CCN(C(*)=O)CC2)C1] | (506) |
| 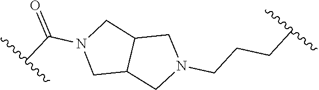 [SMILES: *CCCN1CC2CN(C(*)=O)CC2C1] | (507) |
| 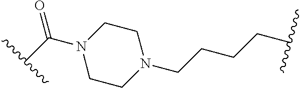 [SMILES: *CCCCN1CCN(C(*)=O)CC1] | (508) |
| 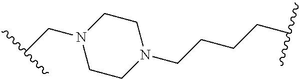 [SMILES: *CCCCN1CCN(C*)CC1] | (509) |
| 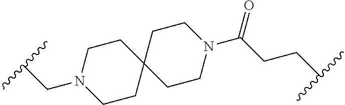 [SMILES: *CCC(=O)N1CCC2(CCN(C*)CC2)CC1] | (510) |
| 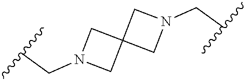 [SMILES: *CN1CC2(C1)CN(C*)C2] | (511) |
| 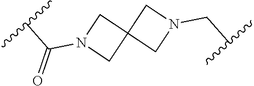 [SMILES: *CN1CC2(C1)CN(C(*)=O)C2] | (512) |
| 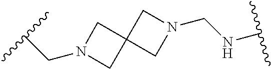 [SMILES: *CN1CC2(C1)CN(CN*)C2] | (513) |
| 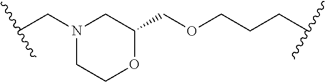 [SMILES: *CCCOC[C@H]1CN(C*)CCO1] | (514) |
| 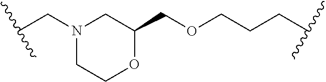 [SMILES: *CCCOC[C@@H]1CN(C*)CCO1] | (515) |
| 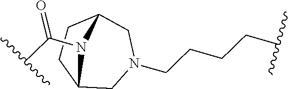 [SMILES: *CCCCN1C[C@H]2CC[C@@H](C1)N2C(*)=O] | (516) |
| 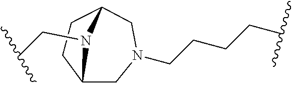 [SMILES: *CCCCN1C[C@H]2CC[C@@H](C1)N2C*] | (517) |
| 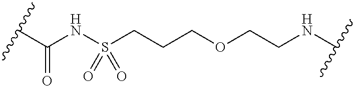 [SMILES: *NCCOCCCS(=O)(=O)NC(*)=O] | (518) |
| 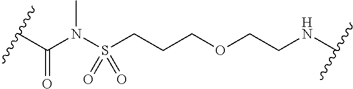 [SMILES: *NCCOCCCS(=O)(=O)N(C)C(*)=O] | (519) |
| 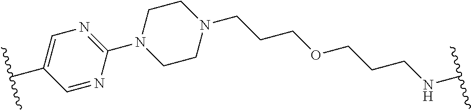 [SMILES: *NCCCOCCCN1CCN(c2ncc(*)cn2)CC1] | (520) |
| 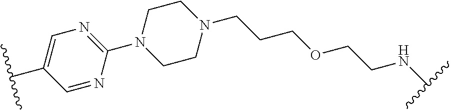 [SMILES: *NCCOCCCN1CCN(c2ncc(*)cn2)CC1] | (521) |
| 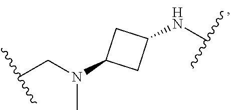 [SMILES: *CN(C)[C@H]1C[C@H](N*)C1] | (522) |
| 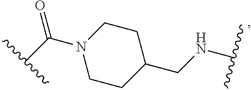 [SMILES: *NCC1CCN(C(*)=O)CC1] | (523) |
| 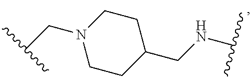 [SMILES: *CN1CCC(CN*)CC1] | (524) |
| 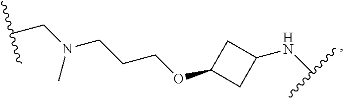 | (525) |
| 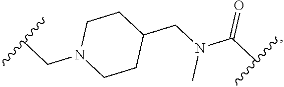 [SMILES: *CN1CCC(CN(C)C(*)=O)CC1] | (526) |
| 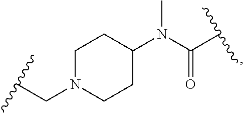 [SMILES: *CN1CCC(N(C)C(*)=O)CC1] | (527) |
| 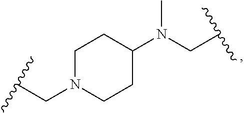 [SMILES: *CN1CCC(N(C)C*)CC1] | (528) |
| 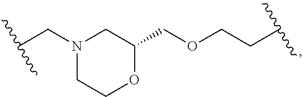 [SMILES: *CCOC[C@H]1CN(C*)CCO1] | (529) |
| 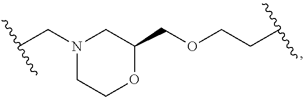 [SMILES: *CCOC[C@@H]1CN(C*)CCO1] | (530) |
| 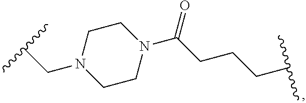 [SMILES: *CCCC(=O)N1CCN(C*)CC1] | (531) |
| 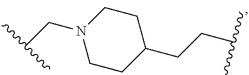 [SMILES: *CCC1CCN(C*)CC1] | (532) |
| 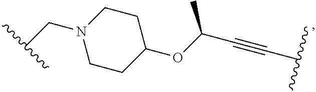 [SMILES: *C#C[C@H](C)OC1CCN(C*)CC1] | (533) |
| 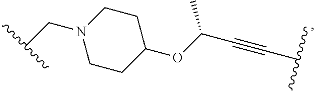 [SMILES: *C#C[C@@H](C)OC1CCN(C*)CC1] | (534) |
| 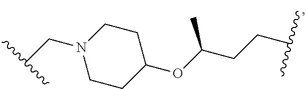 [SMILES: *CC[C@H](C)OC1CCN(C*)CC1] | (535) |
| 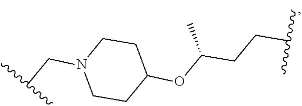 [SMILES: *CC[C@@H](C)OC1CCN(C*)CC1] | (536) |
| 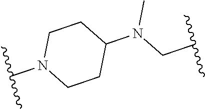 [SMILES: *CN(C)C1CCN(*)CC1] | (537) |
| 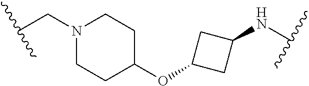 [SMILES: *CN1CCC(O[C@H]2C[C@H](N*)C2)CC1] | (538) |
| 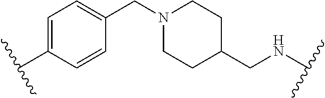 [SMILES: *NCC1CCN(Cc2ccc(*)cc2)CC1] | (539) |
| 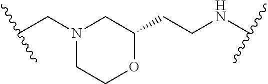 [SMILES: *CN1CCO[C@@H](CCN*)C1] | (540) |
| 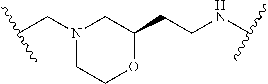 [SMILES: *CN1CCO[C@H](CCN*)C1] | (541) |
| 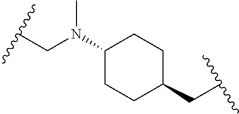 [SMILES: *CN(C)[C@H]1CC[C@H](C*)CC1] | (542) |
| 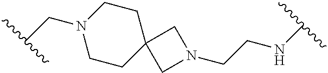 [SMILES: *CN1CCC2(CC1)CN(CCN*)C2] | (543) |
| 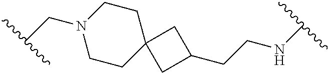 [SMILES: *CN1CCC2(CC1)CC(CCN*)C2] | (544) |
| 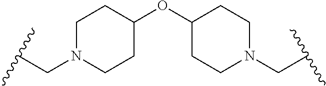 [SMILES: *CN1CCC(OC2CCN(C*)CC2)CC1] | (545) |
| 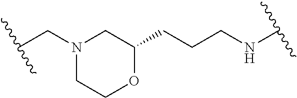 [SMILES: *CN1CCO[C@@H](CCCN*)C1] | (546) |
| 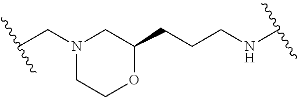 [SMILES: *CN1CCO[C@H](CCCN*)C1] | (547) |
| 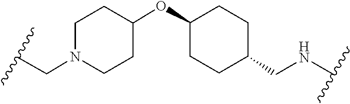 [SMILES: *CN1CCC(O[C@H]2CC[C@H](CN*)CC2)CC1] | (548) |
| 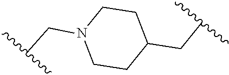 [SMILES: *CC1CCN(C*)CC1] | (549) |
| 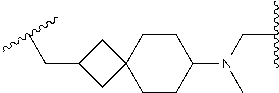 [SMILES: *CC1CC2(CCC(N(C)C*)CC2)C1] | (550) |
| 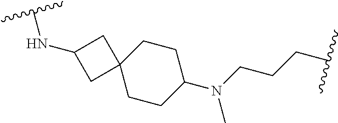 [SMILES: *CCCN(C)C1CCC2(CC1)CC(N*)C2] | (551) |
| 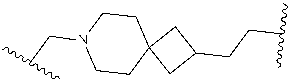 [SMILES: *CCC1CC2(CCN(C*)CC2)C1] | (552) |
| 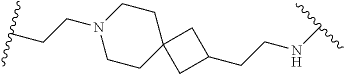 [SMILES: *CCN1CCC2(CC1)CC(CCN*)C2] | (553) |
| 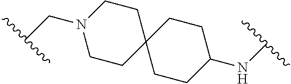 [SMILES: *CN1CCC2(CCC(N*)CC2)CC1] | (554) |
| 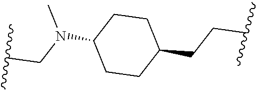 [SMILES: *CC[C@H]1CC[C@H](N(C)C*)CC1] | (555) |
| 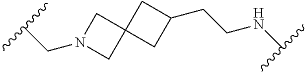 [SMILES: *CN1CC2(CC(CCN*)C2)C1] | (556) |
| 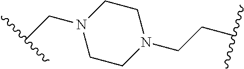 [SMILES: *CCN1CCN(C*)CC1] | (557) |
| 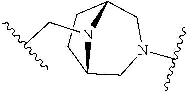 [SMILES: *CN1[C@@H]2CC[C@H]1CN(*)C2] | (558) |
| 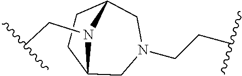 [SMILES: *CCN1C[C@H]2CC[C@@H](C1)N2C*] | (559) |
| 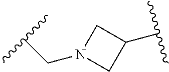 [SMILES: *CN1CC(*)C1] | (560) |
| 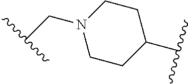 [SMILES: *CN1CCC(*)CC1] | (561) |
| 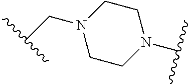 [SMILES: *CN1CCN(*)CC1] | (562) |
| 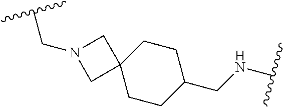 [SMILES: *CN1CC2(CCC(CN*)CC2)C1] | (563) |
| 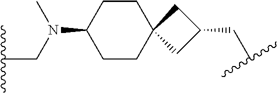 | (564) |
| 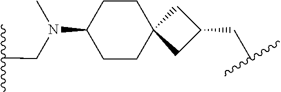 | (565) |
| 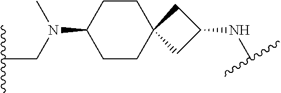 | (566) |
| 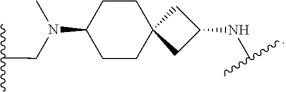 | (567) |
| 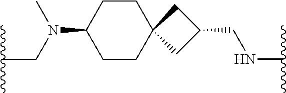 | (568) |
| 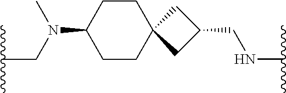 | (569) |
| 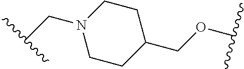 [SMILES: *CN1CCC(CO*)CC1] | (570) |
| 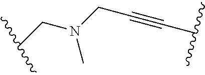 [SMILES: *C#CCN(C)C*] | (571) |
| 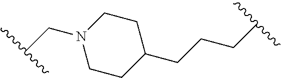 [SMILES: *CCCC1CCN(C*)CC1] | (572) |
| 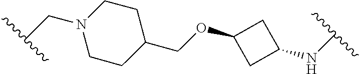 [SMILES: *CN1CCC(CO[C@H]2C[C@H](N*)C2)CC1] | (573) |
| 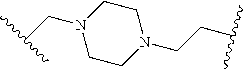 [SMILES: *CCN1CCN(C*)CC1] | (574) |
| 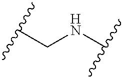 [SMILES: *CN*] | (575) |
| 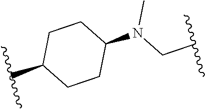 [SMILES: *CN(C)[C@H]1CC[C@@H](*)CC1] | (576) |
| 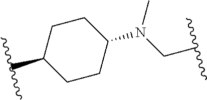 [SMILES: *CN(C)[C@H]1CC[C@H](*)CC1] | (577) |
| 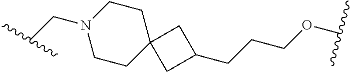 [SMILES: *CN1CCC2(CC1)CC(CCCO*)C2] | (578) |
| 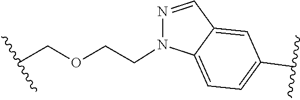 [SMILES: *COCCn1ncc2cc(*)ccc21] | (579) |
| 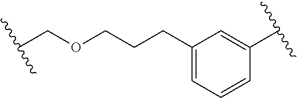 [SMILES: *COCCCc1cccc(*)c1] | (580) |
| 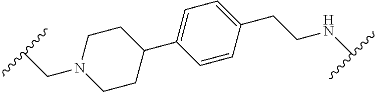 [SMILES: *CN1CCC(c2ccc(CCN*)cc2)CC1] | (581) |
| 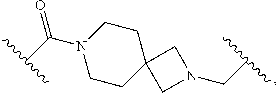 [SMILES: *CN1CC2(CCN(C(*)=O)CC2)C1] | (582) |
| 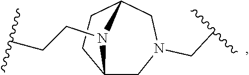 [SMILES: *CCN1[C@@H]2CC[C@H]1CN(C*)C2] | (583) |
| 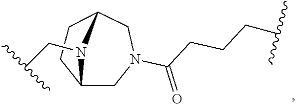 [SMILES: *CCCC(=O)N1C[C@H]2CC[C@@H](C1)N2C*] | (584) |
| 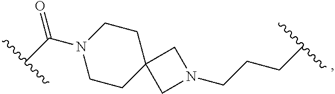 [SMILES: *CCCN1CC2(CCN(C(*)=O)CC2)C1] | (585) |
| 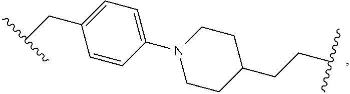 [SMILES: *CCC1CCN(c2ccc(C*)cc2)CC1] | (586) |
| 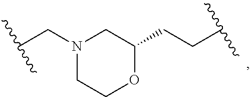 [SMILES: *CC[C@H]1CN(C*)CCO1] | (587) |
| 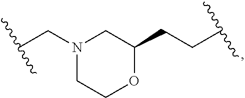 [SMILES: *CC[C@@H]1CN(C*)CCO1] | (588) |
| 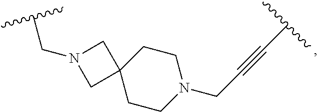 [SMILES: *C#CCN1CCC2(CC1)CN(C*)C2] | (589) |
| 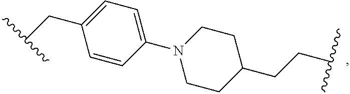 [SMILES: *CCC1CCN(c2ccc(C*)cc2)CC1] | (590) |
| 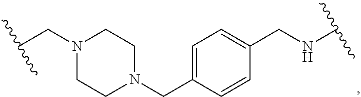 [SMILES: *CN1CCN(Cc2ccc(CN*)cc2)CC1] | (591) |
| 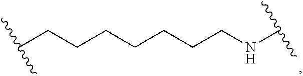 [SMILES: *CCCCCCCN*] | (592) |
| 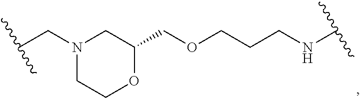 [SMILES: *CN1CCO[C@@H](COCCCN*)C1] | (593) |
| 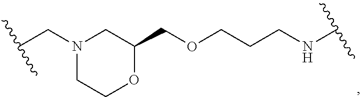 [SMILES: *CN1CCO[C@H](COCCCN*)C1] | (594) |
| 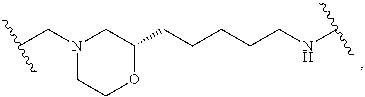 [SMILES: *CN1CCO[C@@H](CCCCCN*)C1] | (595) |
| 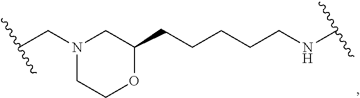 [SMILES: *CN1CCO[C@H](CCCCCN*)C1] | (596) |
| 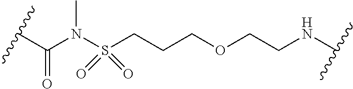 [SMILES: *NCCOCCCS(=O)(=O)N(C)C(*)=O] | (597) |
| 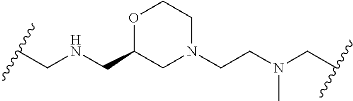 [SMILES: *CNC[C@@H]1CN(CCN(C)C*)CCO1] | (598) |
| 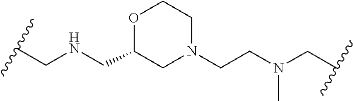 [SMILES: *CNC[C@H]1CN(CCN(C)C*)CCO1] | (599) |
| 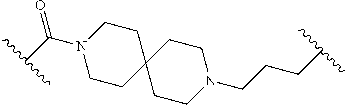 [SMILES: *CCCN1CCC2(CC1)CCN(C(*)=O)CC2] | (600) |
| 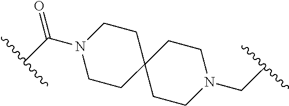 [SMILES: *CN1CCC2(CC1)CCN(C(*)=O)CC2] | (601) |
| 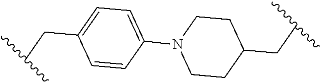 [SMILES: *Cc1ccc(N2CCC(C*)CC2)cc1] | (602) |
| 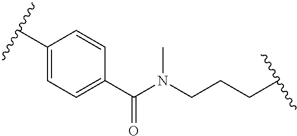 [SMILES: *CCCN(C)C(=O)c1ccc(*)cc1] | (603) |
| 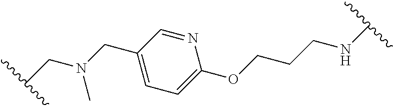 [SMILES: *CN(C)Cc1ccc(OCCCN*)nc1] | (604) |
| 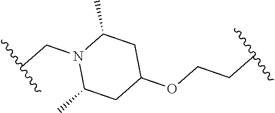 [SMILES: *CCOC1C[C@@H](C)N(C*)[C@@H](C)C1] | (605) |
| 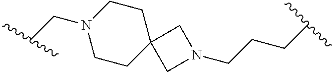 [SMILES: *CCCN1CC2(CCN(C*)CC2)C1] | (606) |
| 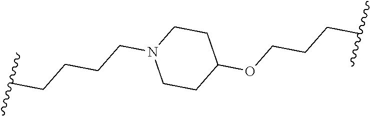 [SMILES: *CCCCN1CCC(OCCC*)CC1] | (607) |
| 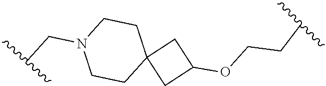 [SMILES: *CCOC1CC2(CCN(C*)CC2)C1] | (608) |
| 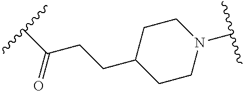 [SMILES: *C(=O)CCC1CCN(*)CC1] | (609) |
| 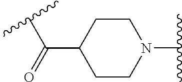 [SMILES: *C(=O)C1CCN(*)CC1] | (610) |
| 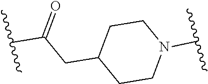 [SMILES: *C(=O)CC1CCN(*)CC1] | (611) |
| 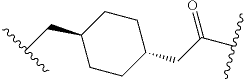 [SMILES: *C[C@H]1CC[C@H](CC(*)=O)CC1] | (612) |
| 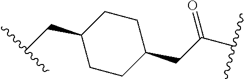 [SMILES: *C[C@H]1CC[C@@H](CC(*)=O)CC1] | (613) |
| 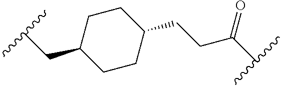 [SMILES: *C[C@H]1CC[C@H](CCC(*)=O)CC1] | (614) |
| 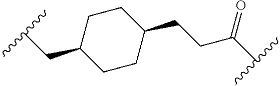 [SMILES: *C[C@H]1CC[C@@H](CCC(*)=O)CC1] | (615) |
| 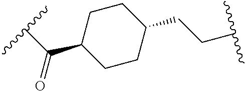 [SMILES: *CC[C@H]1CC[C@H](C(*)=O)CC1] | (616) |
| 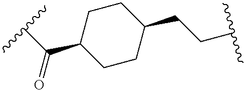 [SMILES: *CC[C@H]1CC[C@@H](C(*)=O)CC1] | (617) |
| 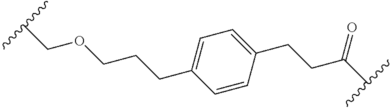 [SMILES: *COCCCc1ccc(CCC(*)=O)cc1] | (618) |
| 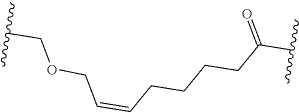 [SMILES: *COC/C=C\CCCCC(*)=O] | (619) |
| 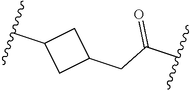 [SMILES: *C(=O)CC1CC(*)C1] | (620) |
| 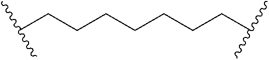 [SMILES: *CCCCCCC*] | (621) |
| 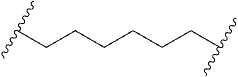 [SMILES: *CCCCCC*] | (622) |
| 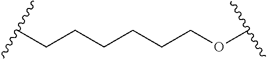 [SMILES: *CCCCCCO*] | (623) |
| 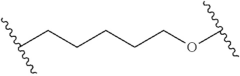 [SMILES: *CCCCCO*] | (624) |
| 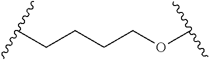 [SMILES: *CCCCO*] | (625) |
| 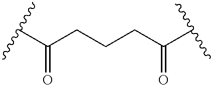 [SMILES: *C(=O)CCCC(*)=O] | (626) |
| 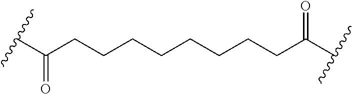 [SMILES: *C(=O)CCCCCCCCC(*)=O] | (627) |
| 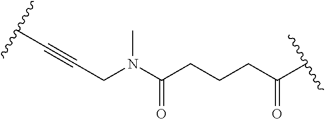 [SMILES: *C#CCN(C)C(=O)CCCC(*)=O] | (628) |
| 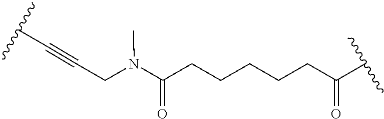 [SMILES: *C#CCN(C)C(=O)CCCCCC(*)=O] | (629) |
| 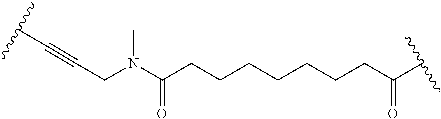 [SMILES: *C#CCN(C)C(=O)CCCCCCCC(*)=O] | (630) |
| 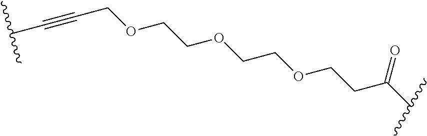 [SMILES: *C#CCOCCOCCOCCC(*)=O] | (631) |
| 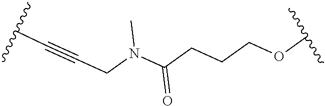 [SMILES: *C#CCN(C)C(=O)CCCO*] | (632) |
| 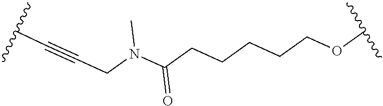 [SMILES: *C#CCN(C)C(=O)CCCCCO*] | (633) |
| 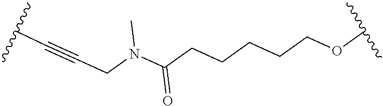 [SMILES: *C#CCN(C)C(=O)CCCCCO*] | (634) |
| 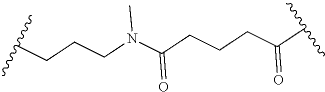 [SMILES: *CCCN(C)C(=O)CCCC(*)=O] | (635) |
| 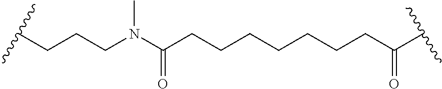 [SMILES: *CCCN(C)C(=O)CCCCCCCC(*)=O] | (636) |
| 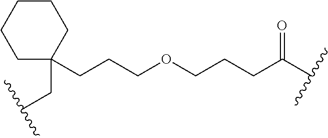 [SMILES: *CC1(CCCOCCCC(*)=O)CCCCC1] | (637) |
| 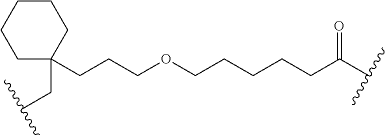 [SMILES: *CC1(CCCOCCCCCC(*)=O)CCCCC1] | (638) |
| 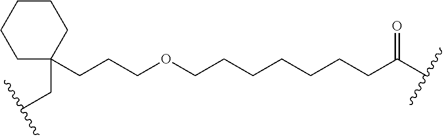 [SMILES: *CC1(CCCOCCCCCCCC(*)=O)CCCCC1] | (639) |
| 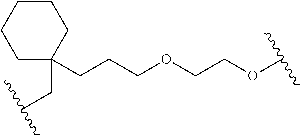 [SMILES: *CC1(CCCOCCO*)CCCCC1] | (640) |
| 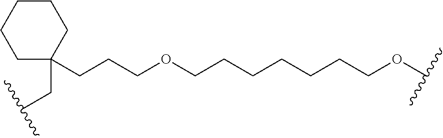 [SMILES: *CC1(CCCOCCCCCCCO*)CCCCC1] | (641) |
| 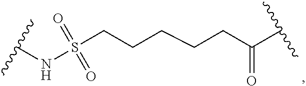 [SMILES: *NS(=O)(=O)CCCCCC(*)=O] | (642) |
| 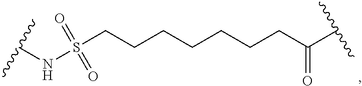 [SMILES: *NS(=O)(=O)CCCCCCCC(*)=O] | (643) |
| 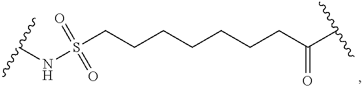 [SMILES: *NS(=O)(=O)CCCCCCCC(*)=O] | (644) |
| 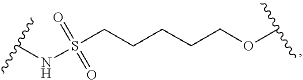 [SMILES: *NS(=O)(=O)CCCCCO*] | (645) |
| 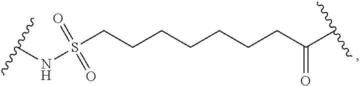 [SMILES: *NS(=O)(=O)CCCCCCCC(*)=O] | (646) |
| 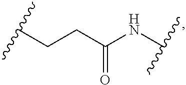 [SMILES: *CCC(=O)N*] | (647) |
| 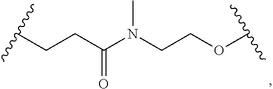 [SMILES: *CCC(=O)N(C)CCO*] | (648) |
| 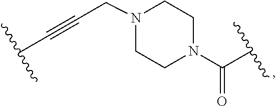 [SMILES: *C#CCN1CCN(C(*)=O)CC1] | (649) |
| 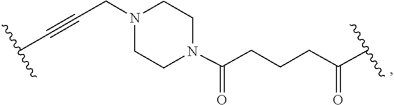 [SMILES: *C#CCN1CCN(C(=O)CCCC(*)=O)CC1] | (650) |
| 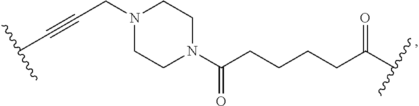 [SMILES: *C#CCN1CCN(C(=O)CCCCC(*)=O)CC1] | (651) |
| 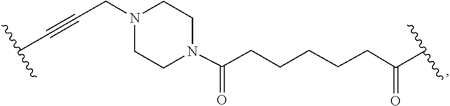 [SMILES: *C#CCN1CCN(C(=O)CCCCCC(*)=O)CC1] | (652) |
| 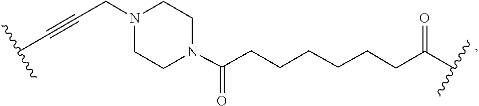 [SMILES: *C#CCN1CCN(C(=O)CCCCCCC(*)=O)CC1] | (653) |
| 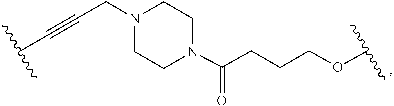 [SMILES: *C#CCN1CCN(C(=O)CCCO*)CC1] | (654) |
| 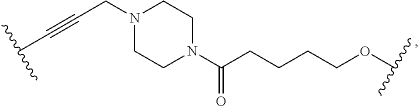 [SMILES: *C#CCN1CCN(C(=O)CCCCO*)CC1] | (655) |
| 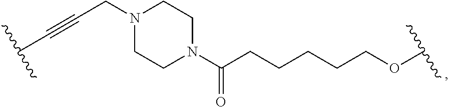 [SMILES: *C#CCN1CCN(C(=O)CCCCCO*)CC1] | (656) |
| 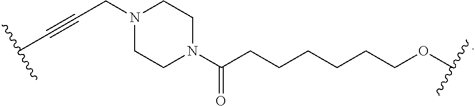 [SMILES: *C#CCN1CCN(C(=O)CCCCCCO*)CC1] | (657) |
| 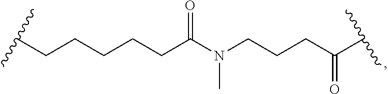 [SMILES: *CCCCCC(=O)N(C)CCCC(*)=O] | (658) |
| 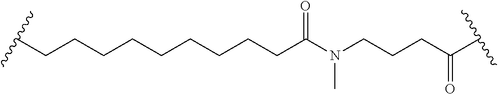 [SMILES: *CCCCCCCCCC(=O)N(C)CCCC(*)=O] | (659) |
| 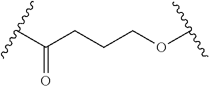 [SMILES: *OCCCC(*)=O] | (660) |
| 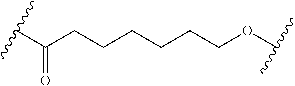 [SMILES: *OCCCCCCC(*)=O] | (661) |
| 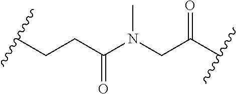 [SMILES: *CCC(=O)N(C)CC(*)=O] | (662) |
| 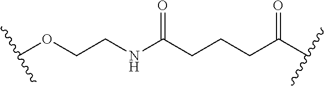 [SMILES: *OCCNC(=O)CCCC(*)=O] | (663) |
| 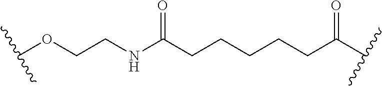 [SMILES: *OCCNC(=O)CCCCCC(*)=O] | (664) |
| 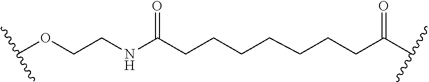 [SMILES: *OCCNC(=O)CCCCCCCC(*)=O] | (665) |
| 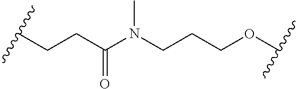 [SMILES: *CCC(=O)N(C)CCCO*] | (666) |
| 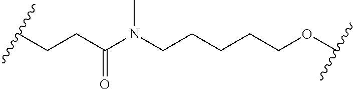 [SMILES: *CCC(=O)N(C)CCCCCO*] | (667) |
| 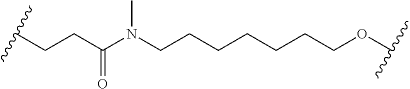 [SMILES: *CCC(=O)N(C)CCCCCCCO*] | (668) |
| 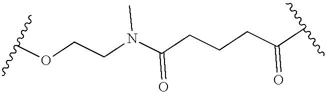 [SMILES: *OCCN(C)C(=O)CCCC(*)=O] | (669) |
| 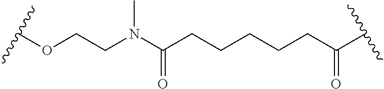 [SMILES: *OCCN(C)C(=O)CCCCCC(*)=O] | (670) |
| 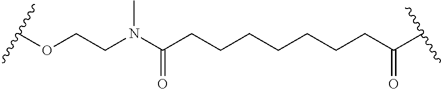 [SMILES: *OCCN(C)C(=O)CCCCCCCC(*)=O] | (671) |
| 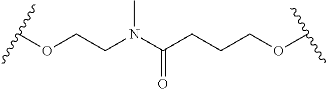 [SMILES: *OCCCC(=O)N(C)CCO*] | (672) |
| 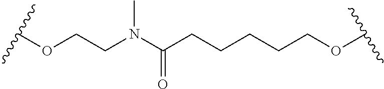 [SMILES: *OCCCCCC(=O)N(C)CCO*] | (673) |
| 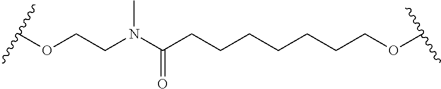 [SMILES: *OCCCCCCCC(=O)N(C)CCO*] | (674) |
| 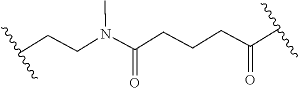 [SMILES: *CCN(C)C(=O)CCCC(*)=O] | (675) |
| 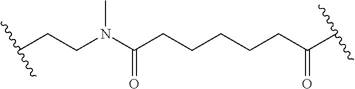 [SMILES: *CCN(C)C(=O)CCCCCC(*)=O] | (676) |
| 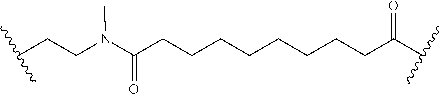 [SMILES: *CCN(C)C(=O)CCCCCCCCC(*)=O] | (677) |
| 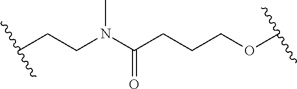 [SMILES: *CCN(C)C(=O)CCCO*] | (678) |
| 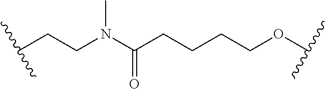 [SMILES: *CCN(C)C(=O)CCCCO*] | (679) |
| 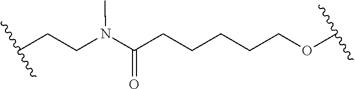 [SMILES: *CCN(C)C(=O)CCCCCO*] | (680) |
| 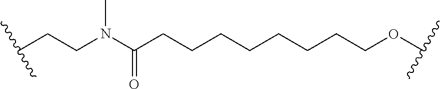 [SMILES: *CCN(C)C(=O)CCCCCCCCO*] | (681) |
| 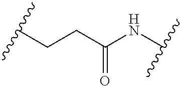 [SMILES: *CCC(=O)N*] | (682) |
| 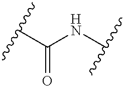 [SMILES: *NC(*)=O] | (683) |
| 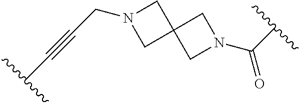 [SMILES: *C#CCN1CC2(C1)CN(C(*)=O)C2] | (684) |
| 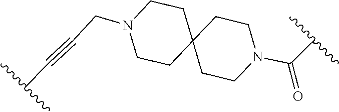 [SMILES: *C#CCN1CCC2(CC1)CCN(C(*)=O)CC2] | (685) |
| 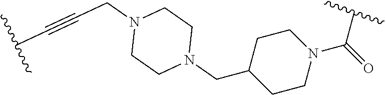 [SMILES: *C#CCN1CCN(CC2CCN(C(*)=O)CC2)CC1] | (686) |
| 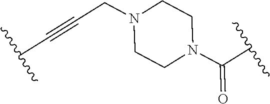 [SMILES: *C#CCN1CCN(C(*)=O)CC1] | (687) |
| 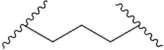 [SMILES: *CCC*] | (688) |
| 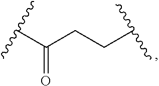 [SMILES: *CCC(*)=O] | (689) |
| 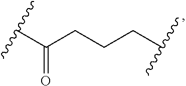 [SMILES: *CCCC(*)=O] | (690) |
| 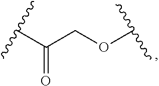 [SMILES: *OCC(*)=O] | (691) |
| 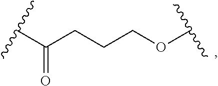 [SMILES: *OCCCC(*)=O] | (692) |
| 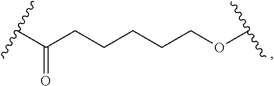 [SMILES: *OCCCCCC(*)=O] | (693) |
| 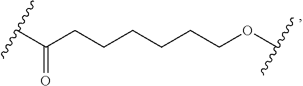 [SMILES: *OCCCCCCC(*)=O] | (694) |
| 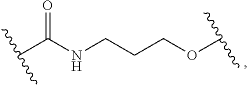 [SMILES: *OCCCNC(*)=O] | (695) |
| 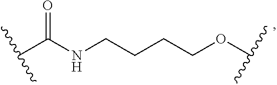 [SMILES: *OCCCCNC(*)=O] | (696) |
| 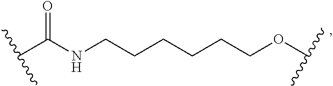 [SMILES: *OCCCCCCNC(*)=O] | (697) |
| 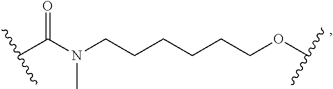 [SMILES: *OCCCCCCN(C)C(*)=O] | (698) |
| 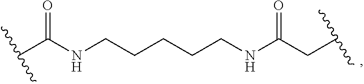 [SMILES: *CC(=O)NCCCCCNC(*)=O] | (699) |
| 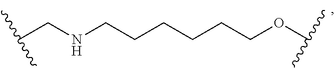 [SMILES: *CNCCCCCCO*] | (700) |
| 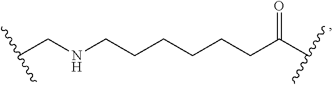 [SMILES: *CNCCCCCCC(*)=O] | (701) |
| 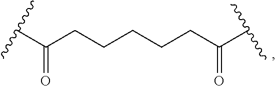 [SMILES: *C(=O)CCCCCC(*)=O] | (702) |
| 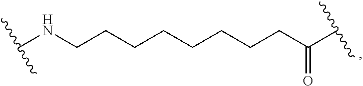 [SMILES: *NCCCCCCCCC(*)=O] | (703) |
| 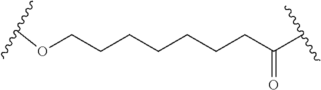 [SMILES: *OCCCCCCCC(*)=O] | (704) |
| 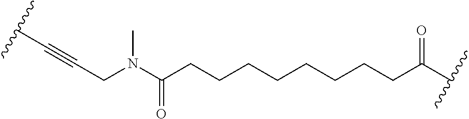 [SMILES: *C#CCN(C)C(=O)CCCCCCCCC(*)=O] | (705) |
| 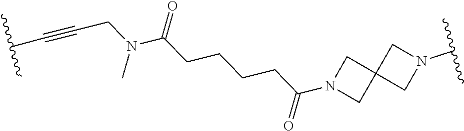 [SMILES: *C#CCN(C)C(=O)CCCCC(=O)N1CC2(CN(*)C2)C1] | (706) |
| 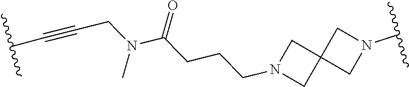 [SMILES: *C#CCN(C)C(=O)CCCN1CC2(CN(*)C2)C1] | (707) |
| 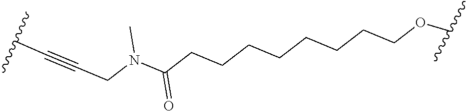 [SMILES: *C#CCN(C)C(=O)CCCCCCCCO*] | (708) |
| 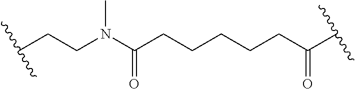 [SMILES: *CCN(C)C(=O)CCCCCC(*)=O] | (709) |
| 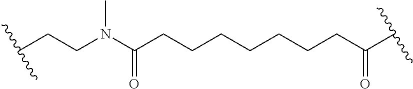 [SMILES: *CCN(C)C(=O)CCCCCCCC(*)=O] | (710) |
| 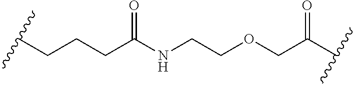 [SMILES: *CCCC(=O)NCCOCC(*)=O] | (711) |
| 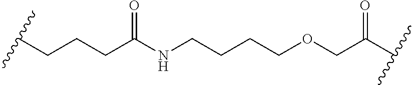 [SMILES: *CCCC(=O)NCCCCOCC(*)=O] | (712) |
| 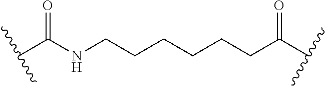 [SMILES: *C(=O)CCCCCCNC(*)=O] | (713) |
| 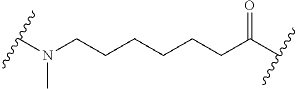 [SMILES: *C(=O)CCCCCCN(*)C] | (714) |
| 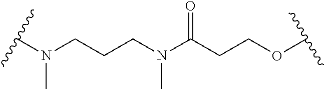 [SMILES: *OCCC(=O)N(C)CCCN(*)C] | (715) |
| 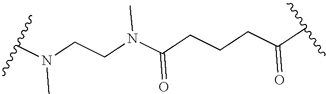 [SMILES: *C(=O)CCCC(=O)N(C)CCN(*)C] | (716) |
| 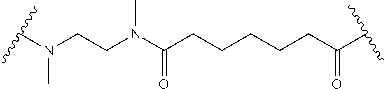 [SMILES: *C(=O)CCCCCC(=O)N(C)CCN(*)C] | (717) |
| 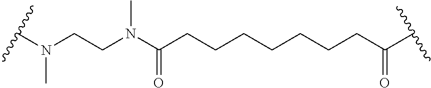 [SMILES: *C(=O)CCCCCCCC(=O)N(C)CCN(*)C] | (718) |
| 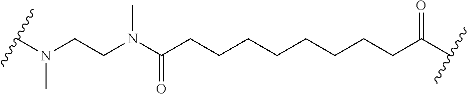 [SMILES: *C(=O)CCCCCCCCC(=O)N(C)CCN(*)C] | (719) |
| 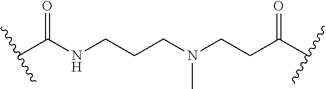 [SMILES: *C(=O)CCN(C)CCCNC(*)=O] | (720) |
| 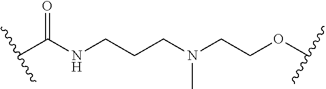 [SMILES: *OCCN(C)CCCNC(*)=O] | (721) |
| 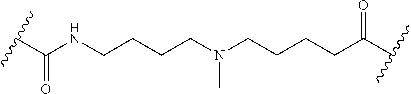 [SMILES: *C(=O)CCCCN(C)CCCCNC(*)=O] | (722) |
| 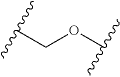 [SMILES: *CO*] | (723) |
| 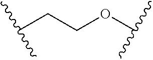 [SMILES: *CCO*] | (724) |
| 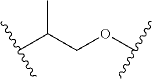 [SMILES: *OCC(*)C] | (725) |
| 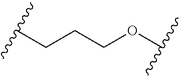 [SMILES: *CCCO*] | (726) |
| 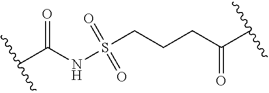 [SMILES: *C(=O)CCCS(=O)(=O)NC(*)=O] | (727) |
| 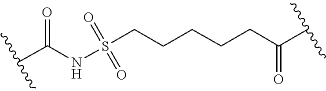 [SMILES: *C(=O)CCCCCS(=O)(=O)NC(*)=O] | (728) |
| 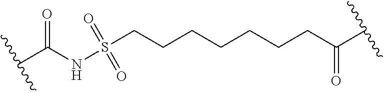 [SMILES: *C(=O)CCCCCCCS(=O)(=O)NC(*)=O] | (729) |
| 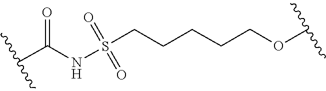 [SMILES: *OCCCCCS(=O)(=O)NC(*)=O] | (730) |
| 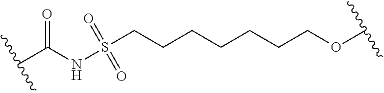 [SMILES: *OCCCCCCCS(=O)(=O)NC(*)=O] | (731) |
| 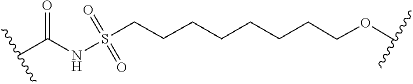 [SMILES: *OCCCCCCCCS(=O)(=O)NC(*)=O] | (732) |
| 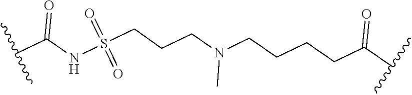 [SMILES: *C(=O)CCCCN(C)CCCS(=O)(=O)NC(*)=O] | (733) |
| 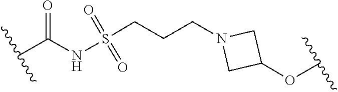 [SMILES: *OC1CN(CCCS(=O)(=O)NC(*)=O)C1] | (734) |
| 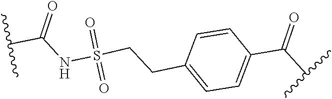 [SMILES: *C(=O)NS(=O)(=O)CCc1ccc(C(*)=O)cc1] | (735) |
| 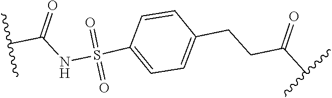 [SMILES: *C(=O)CCc1ccc(S(=O)(=O)NC(*)=O)cc1] | (736) |
| 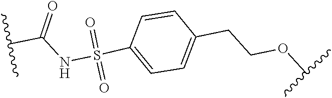 [SMILES: *OCCc1ccc(S(=O)(=O)NC(*)=O)cc1] | (737) |
| 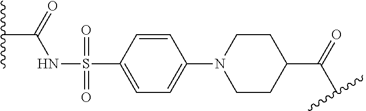 [SMILES: *C(=O)NS(=O)(=O)c1ccc(N2CCC(C(*)=O)CC2)cc1] | (738) |
| 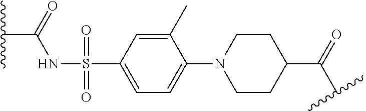 [SMILES: *C(=O)NS(=O)(=O)c1ccc(N2CCC(C(*)=O)CC2)c(C)c1] | (739) |
| 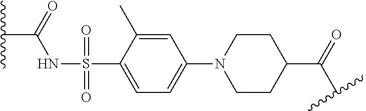 [SMILES: *C(=O)NS(=O)(=O)c1ccc(N2CCC(C(*)=O)CC2)cc1C] | (740) |
| 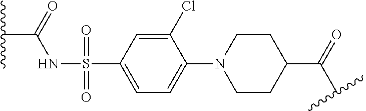 [SMILES: *C(=O)NS(=O)(=O)c1ccc(N2CCC(C(*)=O)CC2)c(Cl)c1] | (741) |
| 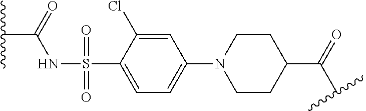 [SMILES: *C(=O)NS(=O)(=O)c1ccc(N2CCC(C(*)=O)CC2)cc1Cl] | (742) |
| 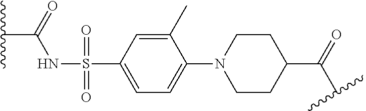 [SMILES: *C(=O)NS(=O)(=O)c1ccc(N2CCC(C(*)=O)CC2)c(C)c1] | (743) |
| 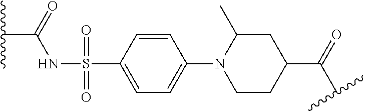 [SMILES: *C(=O)NS(=O)(=O)c1ccc(N2CCC(C(*)=O)CC2C)cc1] | (744) |
| 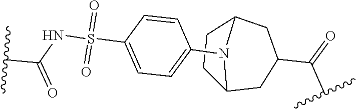 [SMILES: *C(=O)NS(=O)(=O)c1ccc(N2C3CCC2CC(C(*)=O)C3)cc1] | (745) |
| 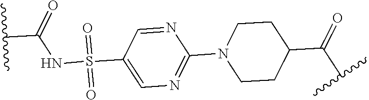 [SMILES: *C(=O)NS(=O)(=O)c1cnc(N2CCC(C(*)=O)CC2)nc1] | (746) |
| 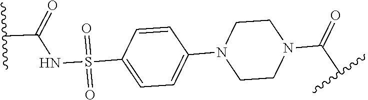 [SMILES: *C(=O)NS(=O)(=O)c1ccc(N2CCN(C(*)=O)CC2)cc1] | (747) |
| 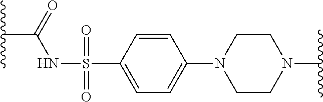 [SMILES: *C(=O)NS(=O)(=O)c1ccc(N2CCN(*)CC2)cc1] | (748) |
| 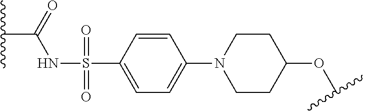 [SMILES: *OC1CCN(c2ccc(S(=O)(=O)NC(*)=O)cc2)CC1] | (749) |
| 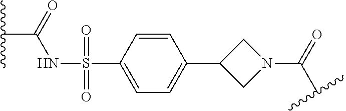 [SMILES: *C(=O)NS(=O)(=O)c1ccc(C2CN(C(*)=O)C2)cc1] | (750) |
| 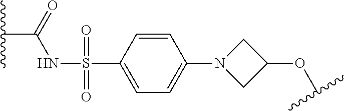 [SMILES: *OC1CN(c2ccc(S(=O)(=O)NC(*)=O)cc2)C1] | (751) |
| 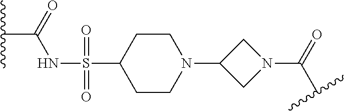 [SMILES: *C(=O)NS(=O)(=O)C1CCN(C2CN(C(*)=O)C2)CC1] | (752) |
| 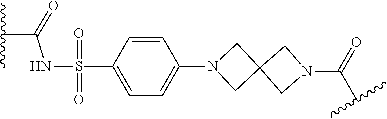 [SMILES: *C(=O)NS(=O)(=O)c1ccc(N2CC3(CN(C(*)=O)C3)C2)cc1] | (753) |
| 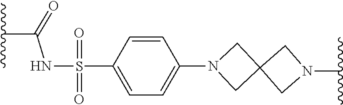 [SMILES: *C(=O)NS(=O)(=O)c1ccc(N2CC3(CN(*)C3)C2)cc1] | (754) |
| 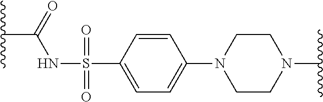 [SMILES: *C(=O)NS(=O)(=O)c1ccc(N2CCN(*)CC2)cc1] | (755) |
| 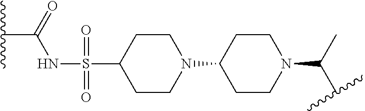 | (756) |
| 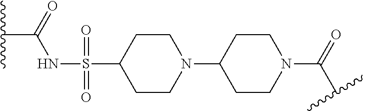 [SMILES: *C(=O)NS(=O)(=O)C1CCN(C2CCN(C(*)=O)CC2)CC1] | (757) |
| 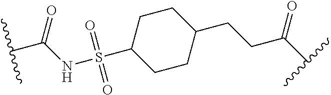 [SMILES: *C(=O)CCC1CCC(S(=O)(=O)NC(*)=O)CC1] | (758) |
| 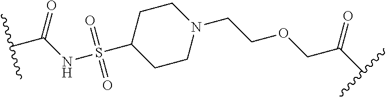 [SMILES: *C(=O)COCCN1CCC(S(=O)(=O)NC(*)=O)CC1] | (759) |
| 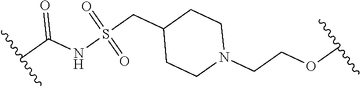 [SMILES: *OCCN1CCC(CS(=O)(=O)NC(*)=O)CC1] | (760) |
| 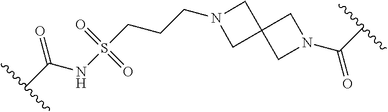 [SMILES: *C(=O)NS(=O)(=O)CCCN1CC2(C1)CN(C(*)=O)C2] | (761) |
| 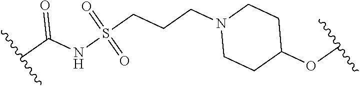 [SMILES: *OC1CCN(CCCS(=O)(=O)NC(*)=O)CC1] | (762) |
| 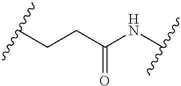 [SMILES: *CCC(=O)N*] | (763) |
| 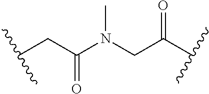 [SMILES: *CC(=O)N(C)CC(*)=O] | (764) |
| 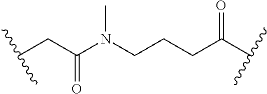 [SMILES: *CC(=O)N(C)CCCC(*)=O] | (765) |
| 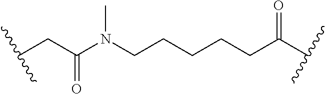 [SMILES: *CC(=O)N(C)CCCCCC(*)=O] | (766) |
| 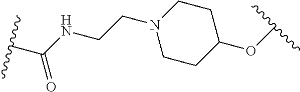 [SMILES: *OC1CCN(CCNC(*)=O)CC1] | (767) |
| 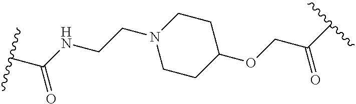 [SMILES: *C(=O)COC1CCN(CCNC(*)=O)CC1] | (768) |
| 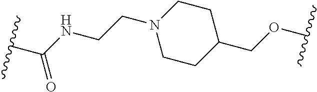 [SMILES: *OCC1CCN(CCNC(*)=O)CC1] | (769) |
| 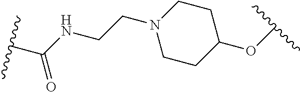 [SMILES: *OC1CCN(CCNC(*)=O)CC1] | (770) |
| 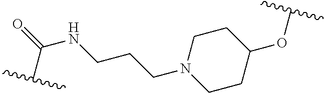 [SMILES: *OC1CCN(CCCNC(*)=O)CC1] | (771) |
| 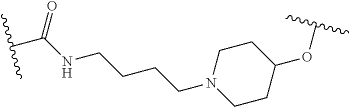 [SMILES: *OC1CCN(CCCCNC(*)=O)CC1] | (772) |
| 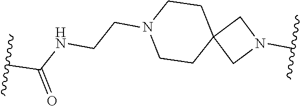 [SMILES: *C(=O)NCCN1CCC2(CC1)CN(*)C2] | (773) |
| 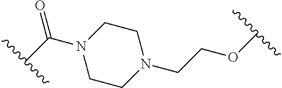 [SMILES: *OCCN1CCN(C(*)=O)CC1] | (774) |
| 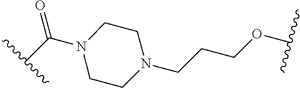 [SMILES: *OCCCN1CCN(C(*)=O)CC1] | (775) |
| 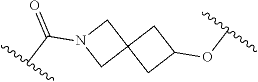 [SMILES: *OC1CC2(C1)CN(C(*)=O)C2] | (776) |
| 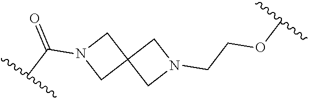 [SMILES: *OCCN1CC2(C1)CN(C(*)=O)C2] | (777) |
| 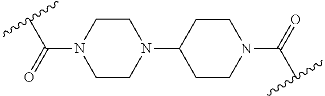 [SMILES: *C(=O)N1CCC(N2CCN(C(*)=O)CC2)CC1] | (778) |
| 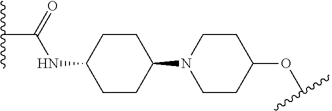 [SMILES: *OC1CCN([C@H]2CC[C@H](NC(*)=O)CC2)CC1] | (779) |
| 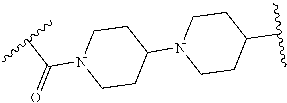 [SMILES: *C(=O)N1CCC(N2CCC(*)CC2)CC1] | (780) |
| 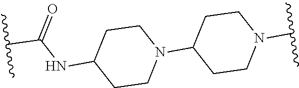 [SMILES: *C(=O)NC1CCN(C2CCN(*)CC2)CC1] | (781) |
| 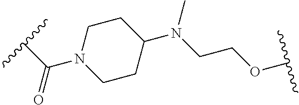 [SMILES: *OCCN(C)C1CCN(C(*)=O)CC1] | (782) |
| 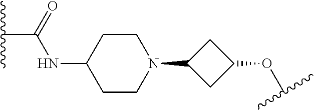 | (783) |
| 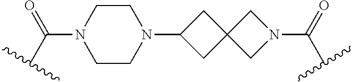 [SMILES: *C(=O)N1CCN(C2CC3(C2)CN(C(*)=O)C3)CC1] | (784) |
| 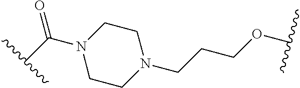 [SMILES: *OCCCN1CCN(C(*)=O)CC1] | (785) |
| 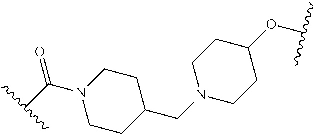 [SMILES: *OC1CCN(CC2CCN(C(*)=O)CC2)CC1] | (786) |
| 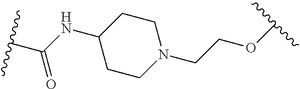 [SMILES: *OCCN1CCC(NC(*)=O)CC1] | (787) |
| 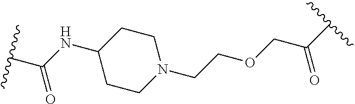 [SMILES: *C(=O)COCCN1CCC(NC(*)=O)CC1] | (788) |
| 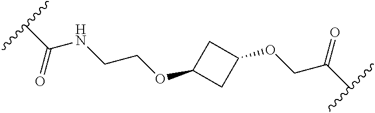 [SMILES: *C(=O)CO[C@H]1C[C@H](OCCNC(*)=O)C1] | (789) |
| 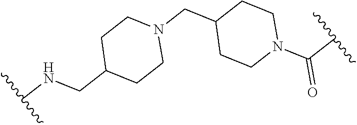 [SMILES: *NCC1CCN(CC2CCN(C(*)=O)CC2)CC1] | (790) |
| 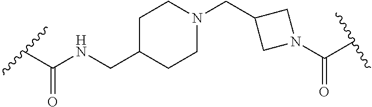 [SMILES: *C(=O)NCC1CCN(CC2CN(C(*)=O)C2)CC1] | (791) |
| 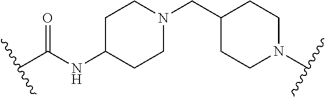 [SMILES: *C(=O)NC1CCN(CC2CCN(*)CC2)CC1] | (792) |
| 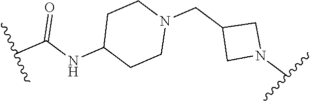 [SMILES: *C(=O)NC1CCN(CC2CN(*)C2)CC1] | (793) |
| 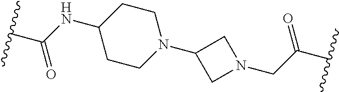 [SMILES: *C(=O)CN1CC(N2CCC(NC(*)=O)CC2)C1] | (794) |
| 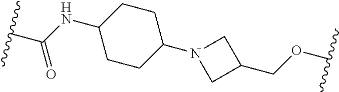 [SMILES: *OCC1CN(C2CCC(NC(*)=O)CC2)C1] | (795) |
| 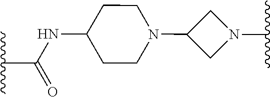 [SMILES: *C(=O)NC1CCN(C2CN(*)C2)CC1] | (796) |
| 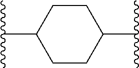 [SMILES: *C1CCC(*)CC1] | (797) |
| 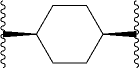 [SMILES: *[C@H]1CC[C@@H](*)CC1] | (798) |
| 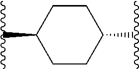 [SMILES: *[C@H]1CC[C@H](*)CC1] | (799) |
| 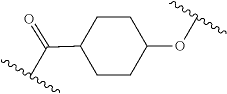 [SMILES: *OC1CCC(C(*)=O)CC1] | (800) |
| 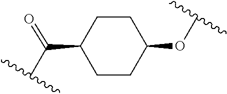 [SMILES: *O[C@H]1CC[C@@H](C(*)=O)CC1] | (801) |
| 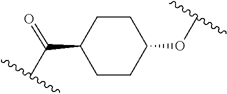 [SMILES: *O[C@H]1CC[C@H](C(*)=O)CC1] | (802) |
| 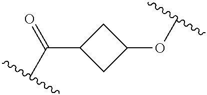 [SMILES: *OC1CC(C(*)=O)C1] | (803) |
| 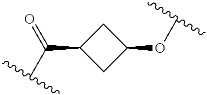 [SMILES: *O[C@H]1C[C@@H](C(*)=O)C1] | (804) |
| 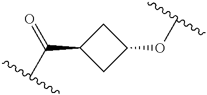 [SMILES: *O[C@H]1C[C@H](C(*)=O)C1] | (805) |
| 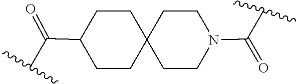 [SMILES: *C(=O)C1CCC2(CC1)CCN(C(*)=O)CC2] | (806) |
| 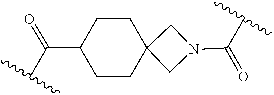 [SMILES: *C(=O)C1CCC2(CC1)CN(C(*)=O)C2] | (807) |
| 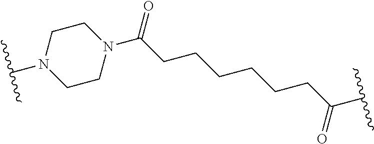 [SMILES: *C(=O)CCCCCCC(=O)N1CCN(*)CC1] | (808) |
| or any one of the linkers described in the Linker section above. | |

In some embodiments, the present invention provides a compound having BBM described and disclosed herein. LBM set forth in Table A above, and a linker set forth in Table B above, or a pharmaceutically acceptable salt thereof.

Exemplary compounds of the invention are set forth in Table 1, below.

TABLE 1
Exemplary Compounds

| I-# | Structure |
|---|---|
| I-1 | 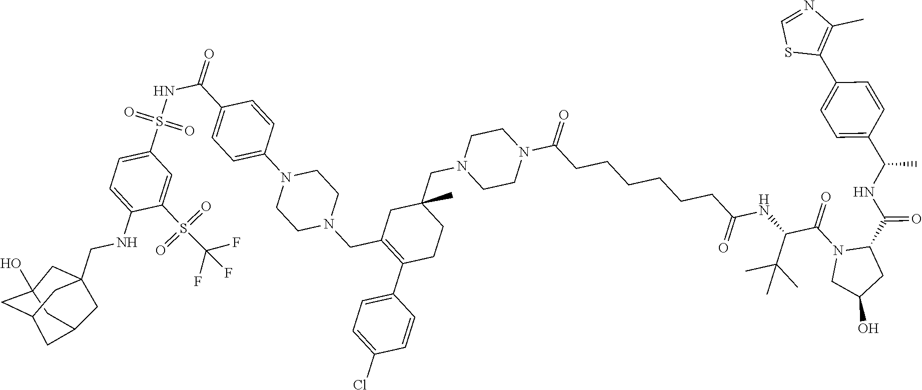 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NCC78CC9CC(CC(O)(C9)C7)C8)c(S(=O)(=O)C(F)(F)F)c6)cc5)CC4)C3)CC2)C(C)(C)C)cc1] |
| I-2 | 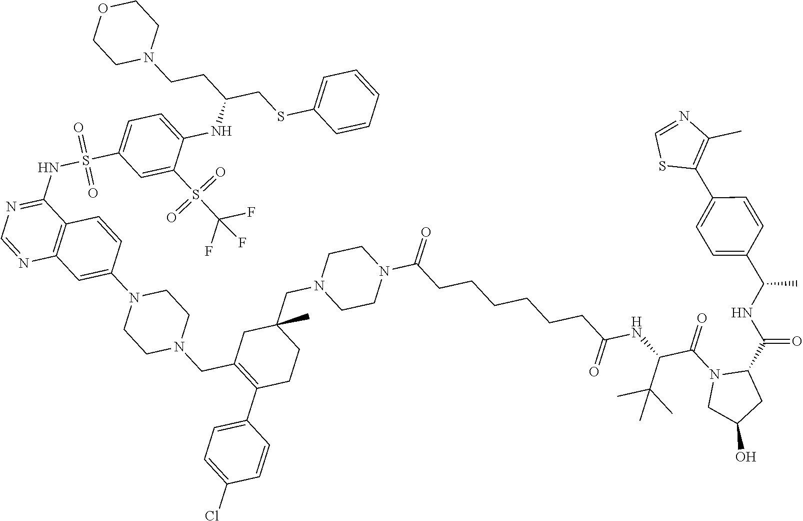 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc6c(NS(=O)(=O)c7ccc(N[C@H](CCN8CCOCC8)CSc8ccccc8)c(S(=O)(=O)C(F)(F)F)c7)ncnc6c5)CC4)C3)CC2)C(C)(C)C)cc1] |
| I-3 | 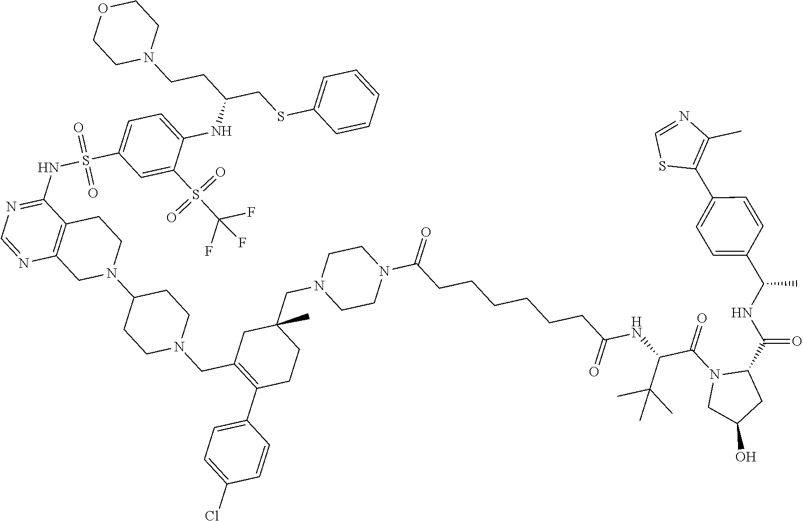 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCC(N5CCc6c(ncnc6NS(=O)(=O)c6ccc(N[C@H](CCN7CCOCC7)CSc7ccccc7)c(S(=O)(=O)C(F)(F)F)c6)C5)CC4)C3)CC2)C(C)(C)C)cc1] |
| I-4 | 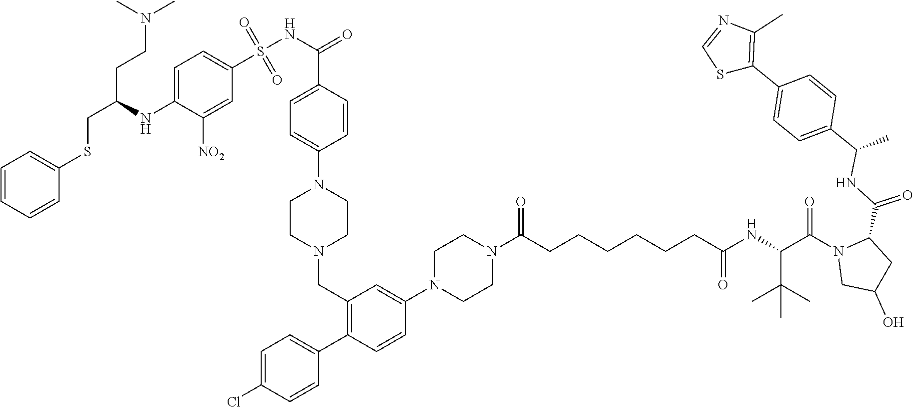 [SMILES: Cc1ncsc1-c1ccc([C@H](C)NC(=O)[C@@H]2CC(O)CN2C(=O)[C@@H](NC(=O)CCCCCCC(=O)N2CCN(c3ccc(-c4ccc(Cl)cc4)c(CN4CCN(c5ccc(C(=O)NS(=O)(=O)c6ccc(N[C@H](CCN(C)C)CSc7ccccc7)c([N+](=O)[O-])c6)cc5)CC4)c3)CC2)C(C)(C)C)cc1] |
| I-5 | 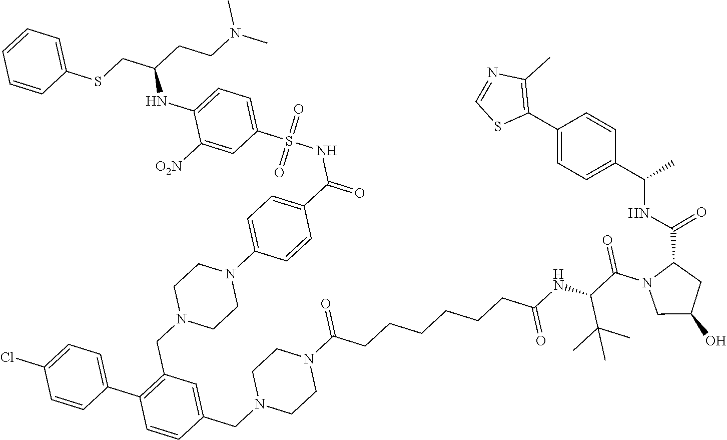 [SMILES: Cc1ncsc1-c1ccc([C@H](C)NC(=O)[C@@H]2C[C@@H](O)CN2C(=O)[C@@H](NC(=O)CCCCCCC(=O)N2CCN(Cc3ccc(-c4ccc(Cl)cc4)c(CN4CCN(c5ccc(C(=O)NS(=O)(=O)c6ccc(N[C@H](CCN(C)C)CSc7ccccc7)c([N+](=O)[O-])c6)cc5)CC4)c3)CC2)C(C)(C)C)cc1] |
| I-6 | 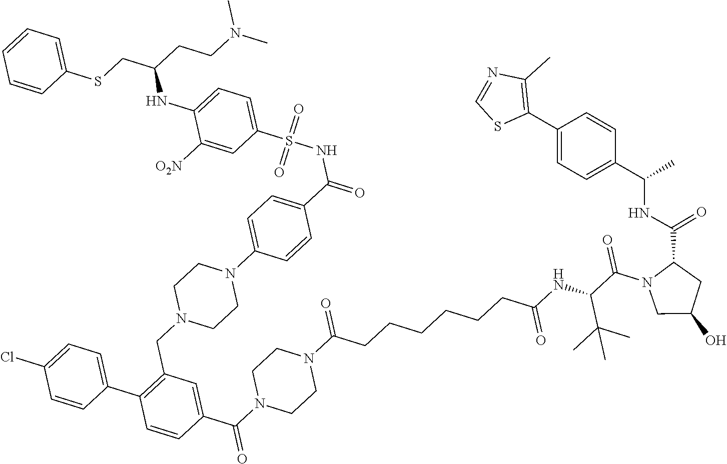 [SMILES: Cc1ncsc1-c1ccc([C@H](C)NC(=O)[C@@H]2C[C@@H](O)CN2C(=O)[C@@H](NC(=O)CCCCCCC(=O)N2CCN(C(=O)c3ccc(-c4ccc(Cl)cc4)c(CN4CCN(c5ccc(C(=O)NS(=O)(=O)c6ccc(N[C@H](CCN(C)C)CSc7ccccc7)c([N+](=O)[O-])c6)cc5)CC4)c3)CC2)C(C)(C)C)cc1] |
| I-7 | 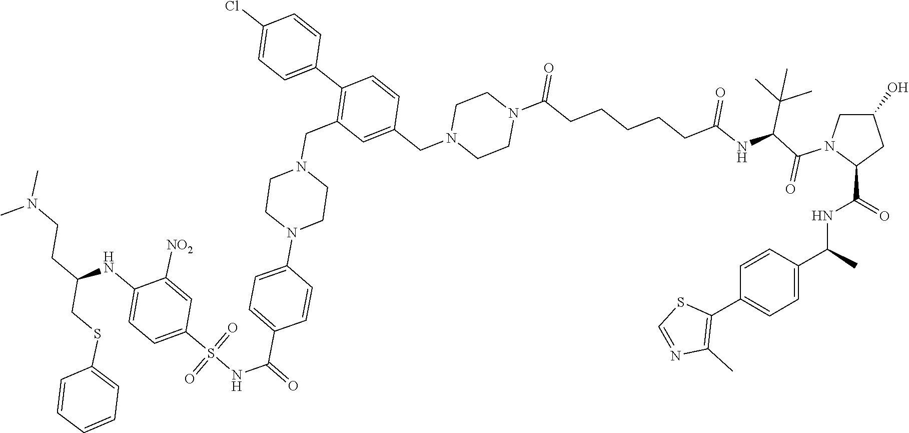 [SMILES: Cc1ncsc1-c1ccc([C@H](C)NC(=O)[C@@H]2C[C@@H](O)CN2C(=O)[C@@H](NC(=O)CCCCCC(=O)N2CCN(Cc3ccc(-c4ccc(Cl)cc4)c(CN4CCN(c5ccc(C(=O)NS(=O)(=O)c6ccc(N[C@H](CCN(C)C)CSc7ccccc7)c([N+](=O)[O-])c6)cc5)CC4)c3)CC2)C(C)(C)C)cc1] |
| I-8 | 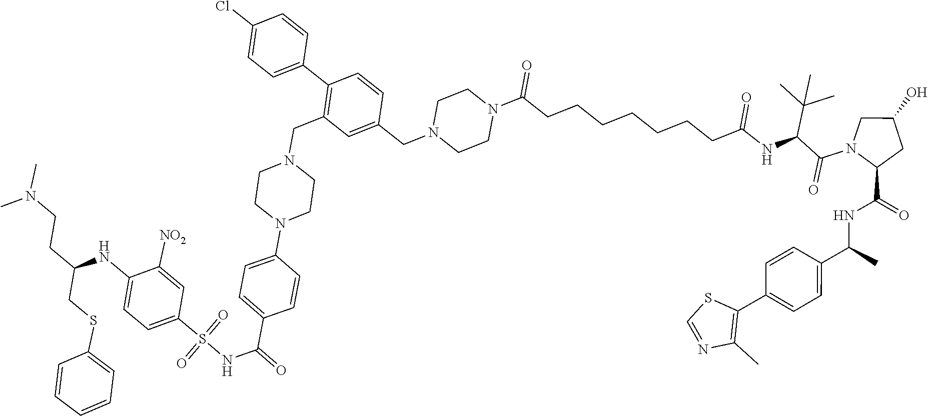 [SMILES: Cc1ncsc1-c1ccc([C@H](C)NC(=O)[C@@H]2C[C@@H](O)CN2C(=O)[C@@H](NC(=O)CCCCCCCC(=O)N2CCN(Cc3ccc(-c4ccc(Cl)cc4)c(CN4CCN(c5ccc(C(=O)NS(=O)(=O)c6ccc(N[C@H](CCN(C)C)CSc7ccccc7)c([N+](=O)[O-])c6)cc5)CC4)c3)CC2)C(C)(C)C)cc1] |
| I-9 | 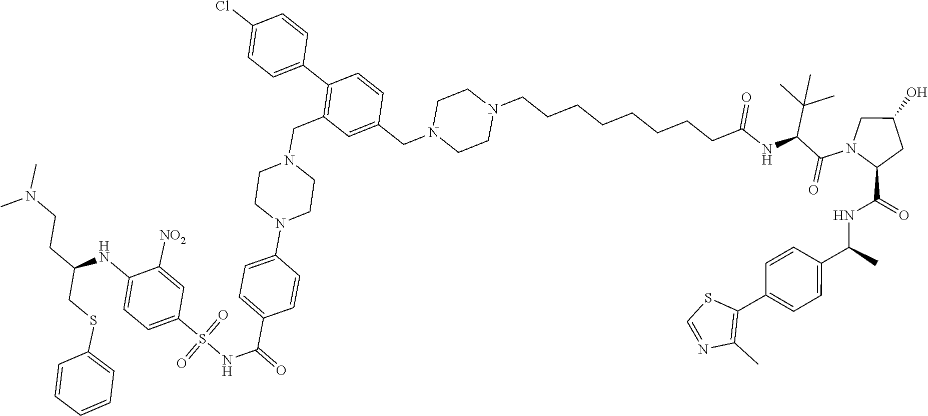 [SMILES: Cc1ncsc1-c1ccc([C@H](C)NC(=O)[C@@H]2C[C@@H](O)CN2C(=O)[C@@H](NC(=O)CCCCCCCCN2CCN(Cc3ccc(-c4ccc(Cl)cc4)c(CN4CCN(c5ccc(C(=O)NS(=O)(=O)c6ccc(N[C@H](CCN(C)C)CSc7ccccc7)c([N+](=O)[O-])c6)cc5)CC4)c3)CC2)C(C)(C)C)cc1] |
| I-10 | 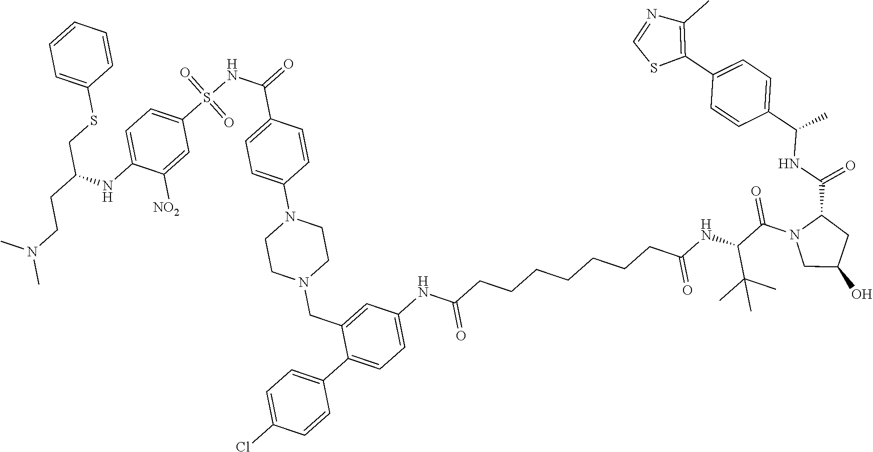 [SMILES: Cc1ncsc1-c1ccc([C@H](C)NC(=O)[C@@H]2C[C@@H](O)CN2C(=O)[C@@H](NC(=O)CCCCCCCC(=O)Nc2ccc(-c3ccc(Cl)cc3)c(CN3CCN(c4ccc(C(=O)NS(=O)(=O)c5ccc(N[C@H](CCN(C)C)CSc6ccccc6)c([N+](=O)[O-])c5)cc4)CC3)c2)C(C)(C)C)cc1] |
| I-11 | 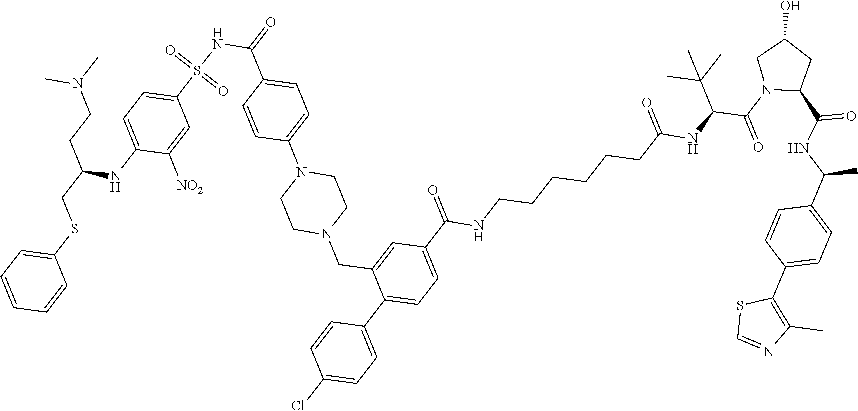 [SMILES: Cc1ncsc1-c1ccc([C@H](C)NC(=O)[C@@H]2C[C@@H](O)CN2C(=O)[C@@H](NC(=O)CCCCCCNC(=O)c2ccc(-c3ccc(Cl)cc3)c(CN3CCN(c4ccc(C(=O)NS(=O)(=O)c5ccc(N[C@H](CCN(C)C)CSc6ccccc6)c([N+](=O)[O-])c5)cc4)CC3)c2)C(C)(C)C)cc1] |
| I-12 | 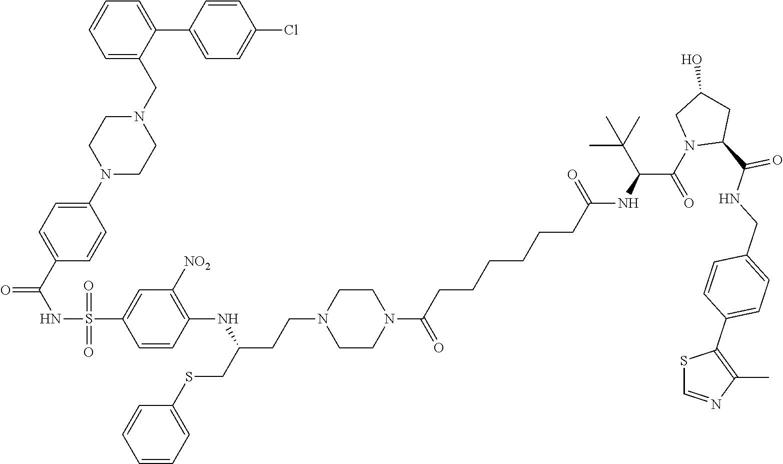 [SMILES: Cc1ncsc1-c1ccc(CNC(=O)[C@@H]2C[C@@H](O)CN2C(=O)[C@@H](NC(=O)CCCCCCC(=O)N2CCN(CC[C@H](CSc3ccccc3)Nc3ccc(S(=O)(=O)NC(=O)c4ccc(N5CCN(Cc6ccccc6-c6ccc(Cl)cc6)CC5)cc4)cc3[N+](=O)[O-])CC2)C(C)(C)C)cc1] |
| I-13 | 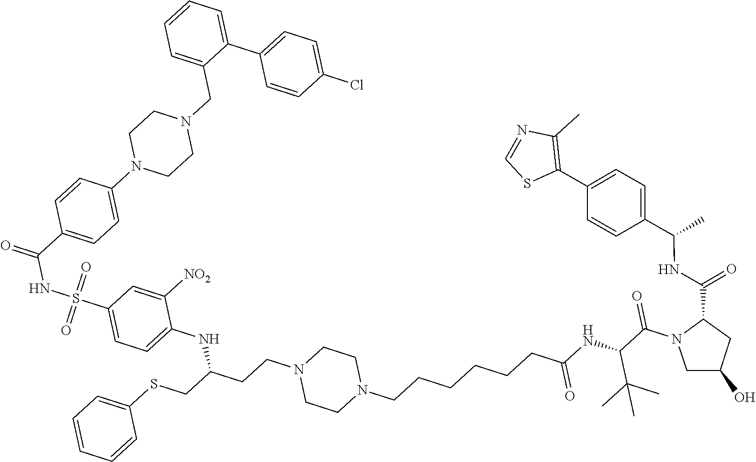 [SMILES: Cc1ncsc1-c1ccc([C@H](C)NC(=O)[C@@H]2C[C@@H](O)CN2C(=O)[C@@H](NC(=O)CCCCCCN2CCN(CC[C@H](CSc3ccccc3)Nc3ccc(S(=O)(=O)NC(=O)c4ccc(N5CCN(Cc6ccccc6-c6ccc(Cl)cc6)CC5)cc4)cc3[N+](=O)[O-])CC2)C(C)(C)C)cc1] |
| I-14 | 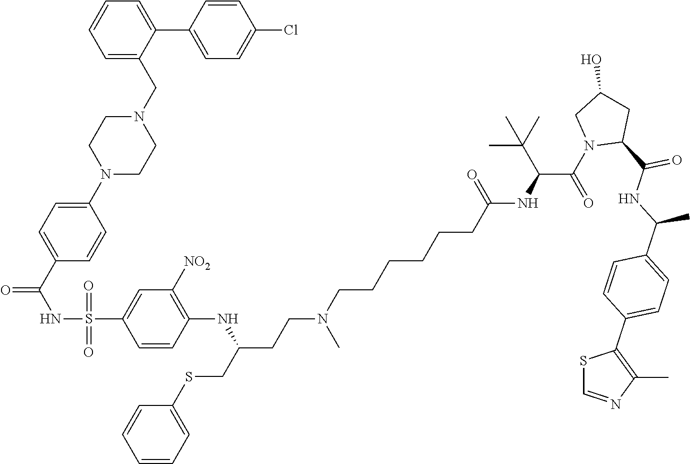 [SMILES: Cc1ncsc1-c1ccc([C@H](C)NC(=O)[C@@H]2C[C@@H](O)CN2C(=O)[C@@H](NC(=O)CCCCCCN(C)CC[C@H](CSc2ccccc2)Nc2ccc(S(=O)(=O)NC(=O)c3ccc(N4CCN(Cc5ccccc5-c5ccc(Cl)cc5)CC4)cc3)cc2[N+](=O)[O-])C(C)(C)C)cc1] |
| I-15 | 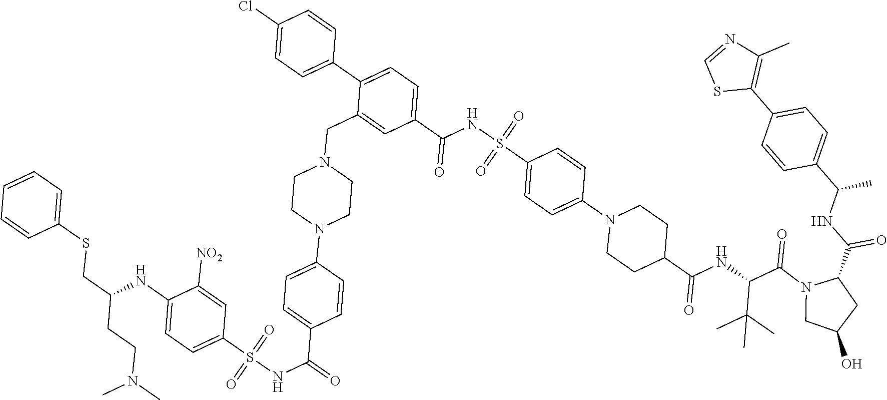 [SMILES: Cc1ncsc1-c1ccc([C@H](C)NC(=O)[C@@H]2C[C@@H](O)CN2C(=O)[C@@H](NC(=O)C2CCN(c3ccc(S(=O)(=O)NC(=O)c4ccc(-c5ccc(Cl)cc5)c(CN5CCN(c6ccc(C(=O)NS(=O)(=O)c7ccc(N[C@H](CCN(C)C)CSc8ccccc8)c([N+](=O)[O-])c7)cc6)CC5)c4)cc3)CC2)C(C)(C)C)cc1] |
| I-16 | 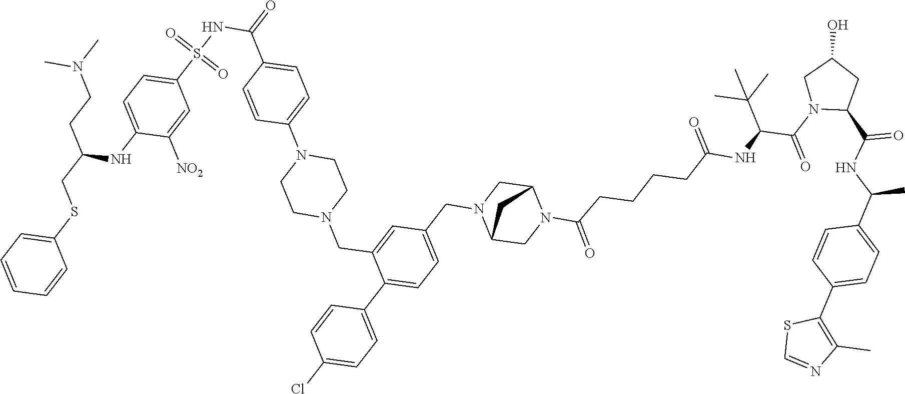 [SMILES: Cc1ncsc1-c1ccc([C@H](C)NC(=O)[C@@H]2C[C@@H](O)CN2C(=O)[C@@H](NC(=O)CCCCC(=O)N2C[C@H]3C[C@@H]2CN3Cc2ccc(-c3ccc(Cl)cc3)c(CN3CCN(c4ccc(C(=O)NS(=O)(=O)c5ccc(N[C@H](CCN(C)C)CSc6ccccc6)c([N+](=O)[O-])c5)cc4)CC3)c2)C(C)(C)C)cc1] |
| I-17 | 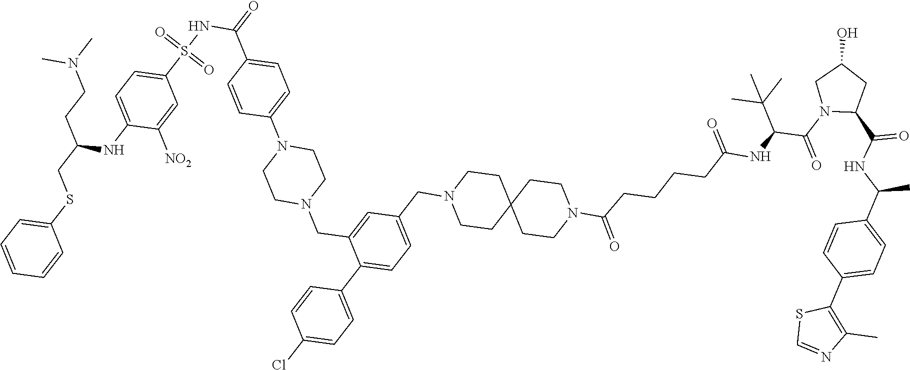 [SMILES: Cc1ncsc1-c1ccc([C@H](C)NC(=O)[C@@H]2C[C@@H](O)CN2C(=O)[C@@H](NC(=O)CCCCC(=O)N2CCC3(CCN(Cc4ccc(-c5ccc(Cl)cc5)c(CN5CCN(c6ccc(C(=O)NS(=O)(=O)c7ccc(N[C@H](CCN(C)C)CSc8ccccc8)c([N+](=O)[O-])c7)cc6)CC5)c4)CC3)CC2)C(C)(C)C)cc1] |
| I-18 | 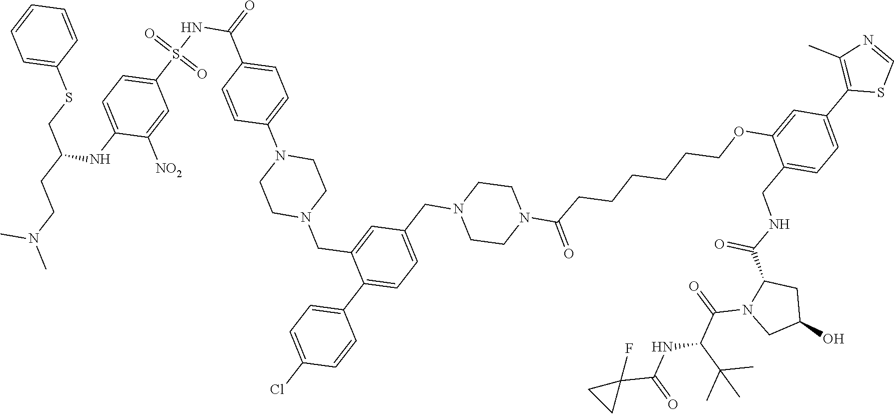 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCC(=O)N2CCN(Cc3ccc(-c4ccc(Cl)cc4)c(CN4CCN(c5ccc(C(=O)NS(=O)(=O)c6ccc(N[C@H](CCN(C)C)CSc7ccccc7)c([N+](=O)[O-])c6)cc5)CC4)c3)CC2)c1] |
| I-19 | 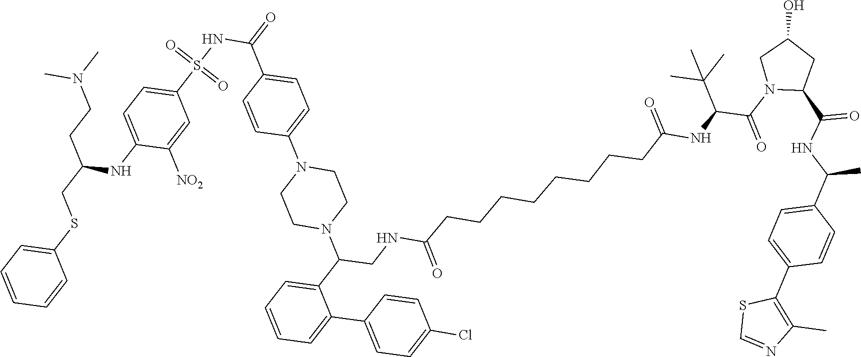 [SMILES: Cc1ncsc1-c1ccc([C@H](C)NC(=O)[C@@H]2C[C@@H](O)CN2C(=O)[C@@H](NC(=O)CCCCCCCCC(=O)NCC(c2ccccc2-c2ccc(Cl)cc2)N2CCN(c3ccc(C(=O)NS(=O)(=O)c4ccc(N[C@H](CCN(C)C)CSc5ccccc5)c([N+](=O)[O-])c4)cc3)CC2)C(C)(C)C)cc1] |
| I-20 | 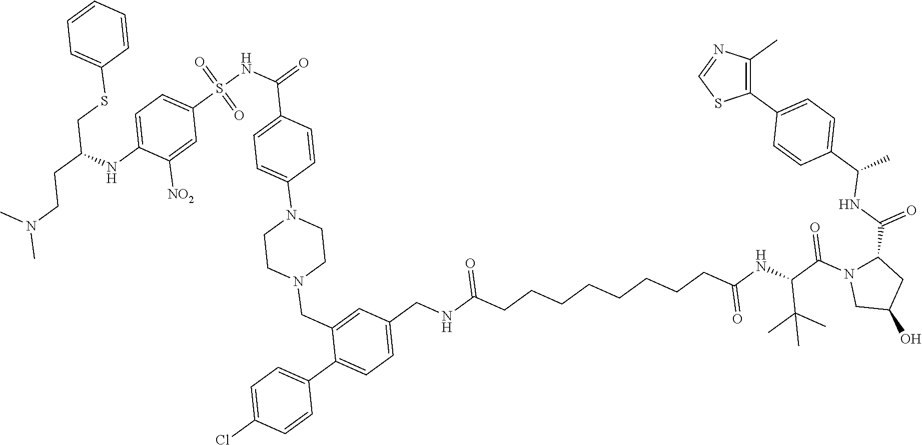 [SMILES: Cc1ncsc1-c1ccc([C@H](C)NC(=O)[C@@H]2C[C@@H](O)CN2C(=O)[C@@H](NC(=O)CCCCCCCCC(=O)NCc2ccc(-c3ccc(Cl)cc3)c(CN3CCN(c4ccc(C(=O)NS(=O)(=O)c5ccc(N[C@H](CCN(C)C)CSc6ccccc6)c([N+](=O)[O-])c5)cc4)CC3)c2)C(C)(C)C)cc1] |
| I-21 | 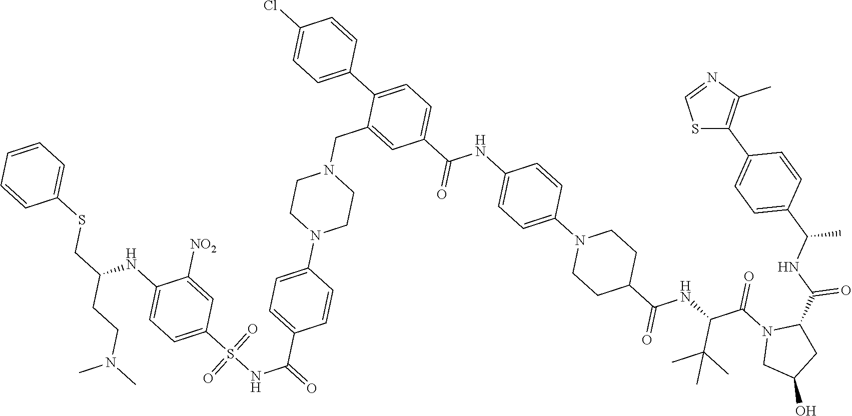 [SMILES: Cc1ncsc1-c1ccc([C@H](C)NC(=O)[C@@H]2C[C@@H](O)CN2C(=O)[C@@H](NC(=O)C2CCN(c3ccc(NC(=O)c4ccc(-c5ccc(Cl)cc5)c(CN5CCN(c6ccc(C(=O)NS(=O)(=O)c7ccc(N[C@H](CCN(C)C)CSc8ccccc8)c([N+](=O)[O-])c7)cc6)CC5)c4)cc3)CC2)C(C)(C)C)cc1] |
| I-22 | 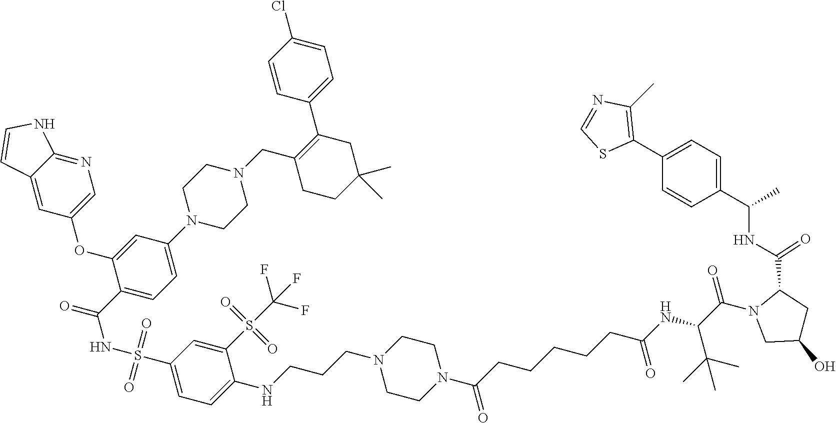 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-23 | 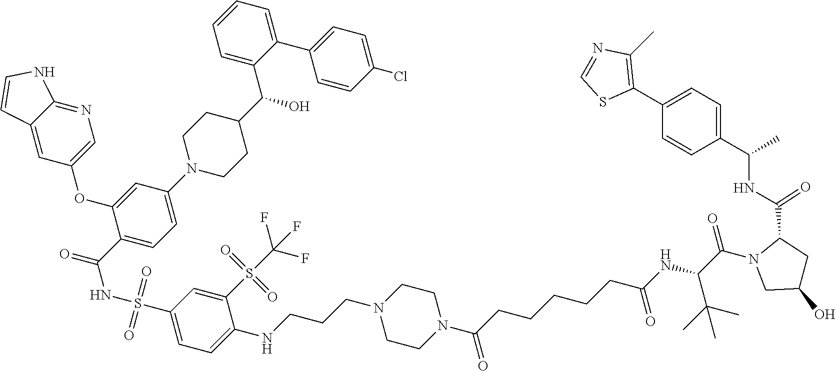 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCC([C@@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-24 | 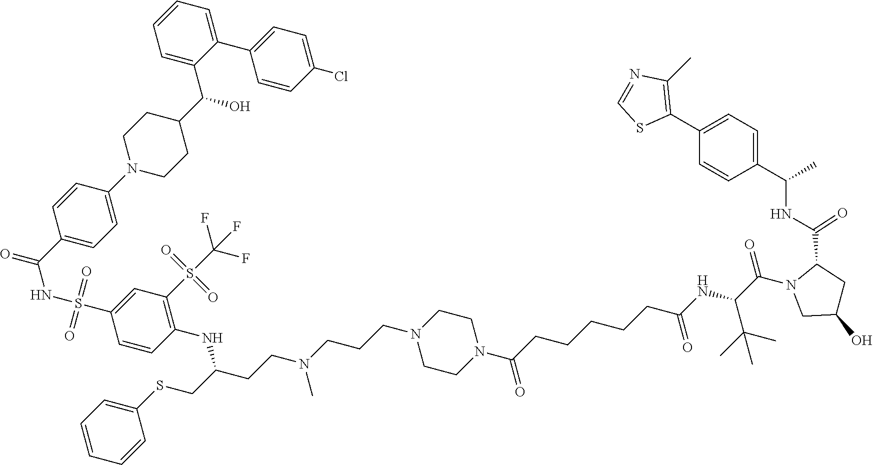 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCN(C)CC[C@H](CSc3ccccc3)Nc3ccc(S(=O)(=O)NC(=O)c4ccc(N5CCC([C@@H](O)c6ccccc6-c6ccc(Cl)cc6)CC5)cc4)cc3S(=O)(=O)C(F)(F)F)CC2)C(C)(C)C)cc1] |
| I-25 | 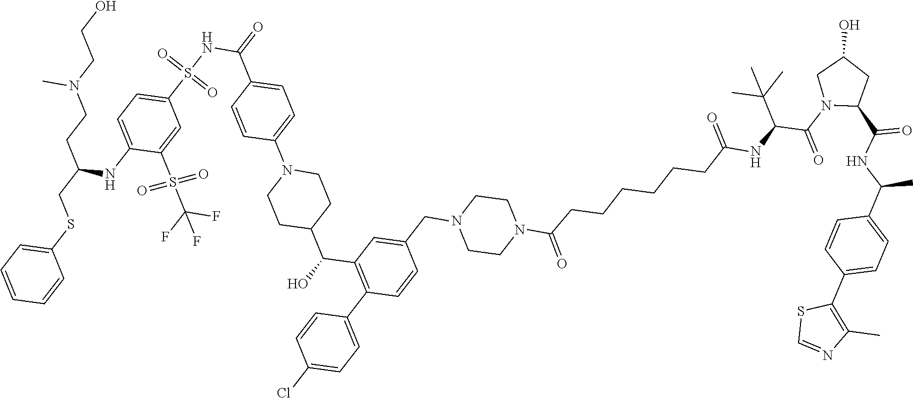 [SMILES: Cc1ncsc1-c1ccc([C@H](C)NC(=O)[C@@H]2C[C@@H](O)CN2C(=O)[C@@H](NC(=O)CCCCCCC(=O)N2CCN(Cc3ccc(-c4ccc(Cl)cc4)c([C@H](O)C4CCN(c5ccc(C(=O)NS(=O)(=O)c6ccc(N[C@H](CCN(C)CCO)CSc7ccccc7)c(S(=O)(=O)C(F)(F)F)c6)cc5)CC4)c3)CC2)C(C)(C)C)cc1] |
| I-26 | 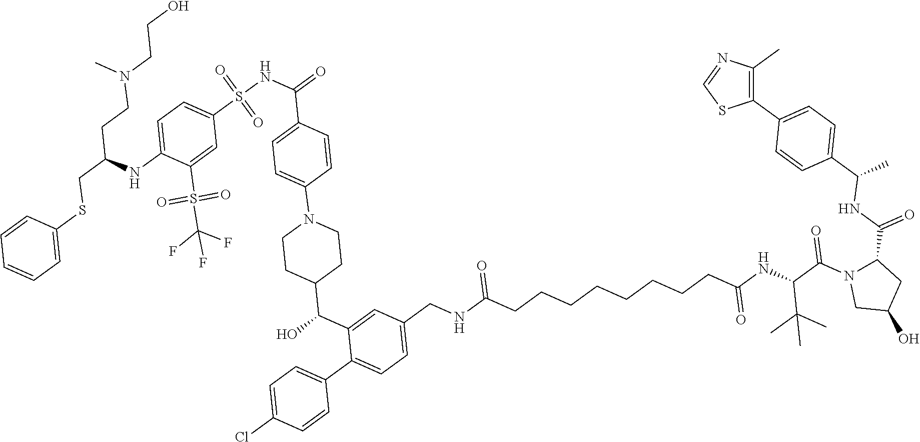 [SMILES: Cc1ncsc1-c1ccc([C@H](C)NC(=O)[C@@H]2C[C@@H](O)CN2C(=O)[C@@H](NC(=O)CCCCCCCCC(=O)NCc2ccc(-c3ccc(Cl)cc3)c([C@H](O)C3CCN(c4ccc(C(=O)NS(=O)(=O)c5ccc(N[C@H](CCN(C)CCO)CSc6ccccc6)c(S(=O)(=O)C(F)(F)F)c5)cc4)CC3)c2)C(C)(C)C)cc1] |
| I-27 | 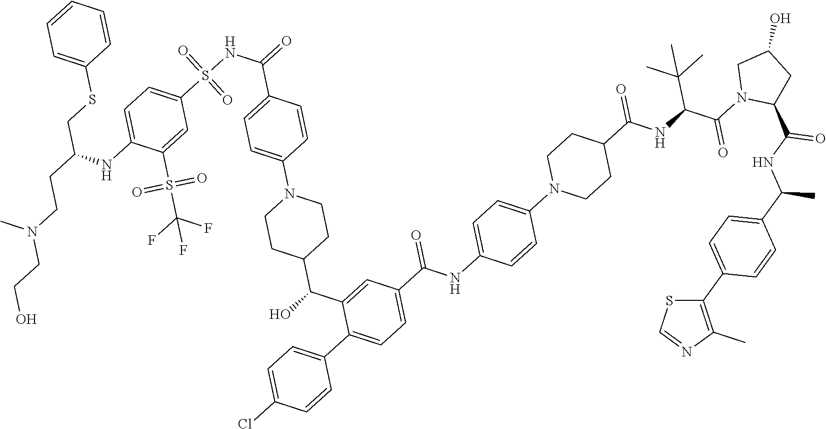 [SMILES: Cc1ncsc1-c1ccc([C@H](C)NC(=O)[C@@H]2C[C@@H](O)CN2C(=O)[C@@H](NC(=O)C2CCN(c3ccc(NC(=O)c4ccc(-c5ccc(Cl)cc5)c([C@H](O)C5CCN(c6ccc(C(=O)NS(=O)(=O)c7ccc(N[C@H](CCN(C)CCO)CSc8ccccc8)c(S(=O)(=O)C(F)(F)F)c7)cc6)CC5)c4)cc3)CC2)C(C)(C)C)cc1] |
| I-28 | 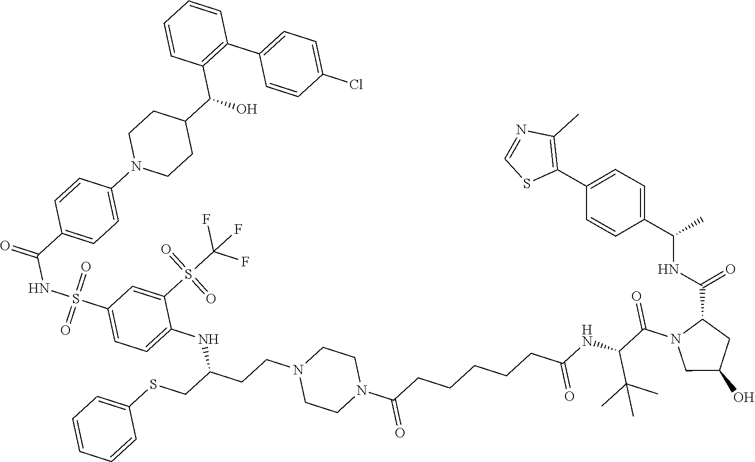 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCC([C@@H](O)c6ccccc6-c6ccc(Cl)cc6)CC5)cc4)cc3S(=O)(=O)C(F)(F)F)CC2)C(C)(C)C)cc1] |
| I-29 | 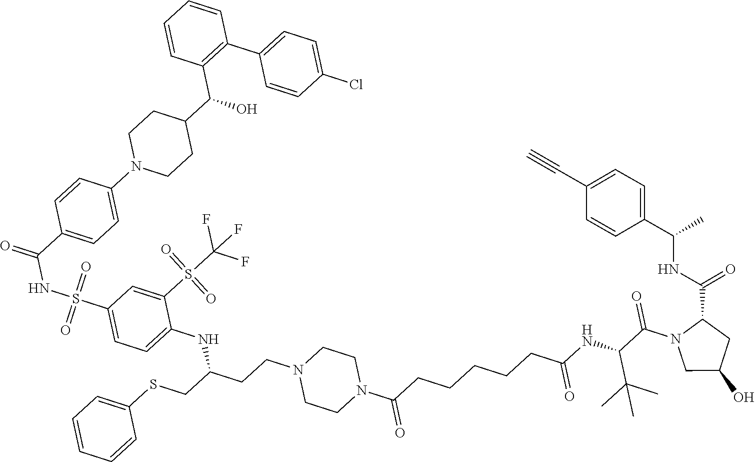 [SMILES: C#Cc1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCC([C@@H](O)c6ccccc6-c6ccc(Cl)cc6)CC5)cc4)cc3S(=O)(=O)C(F)(F)F)CC2)C(C)(C)C)cc1] |
| I-30 | 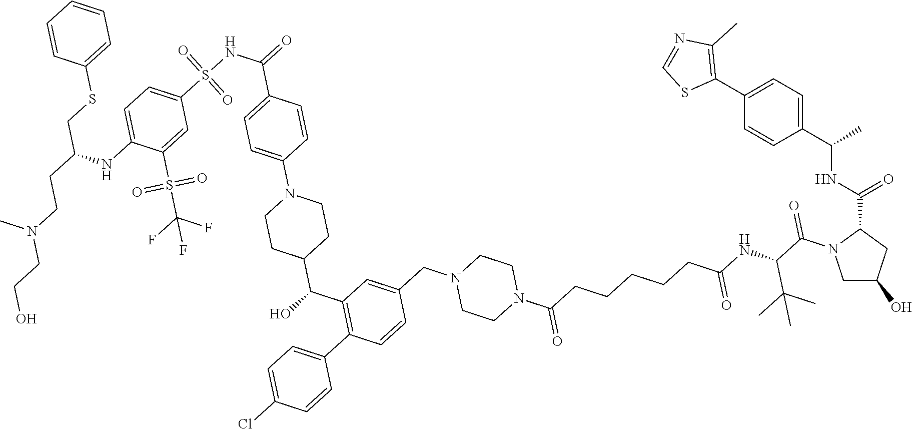 [SMILES: Cc1ncsc1-c1ccc([C@H](C)NC(=O)[C@@H]2C[C@@H](O)CN2C(=O)[C@@H](NC(=O)CCCCCC(=O)N2CCN(Cc3ccc(-c4ccc(Cl)cc4)c([C@H](O)C4CCN(c5ccc(C(=O)NS(=O)(=O)c6ccc(N[C@H](CCN(C)CCO)CSc7ccccc7)c(S(=O)(=O)C(F)(F)F)c6)cc5)CC4)c3)CC2)C(C)(C)C)cc1] |
| I-31 | 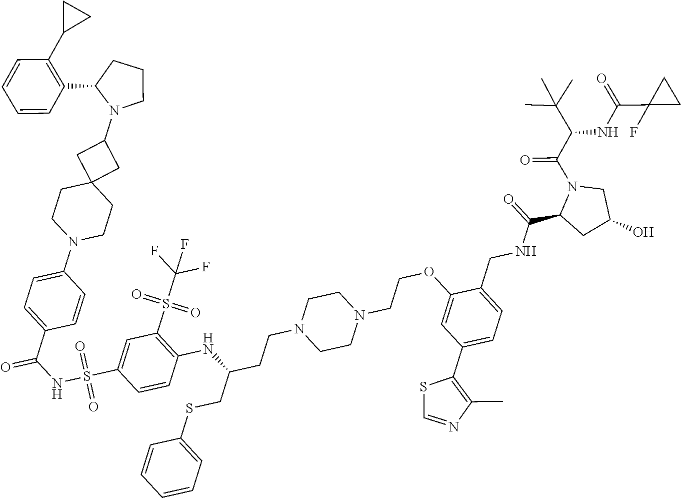 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCN(CC[C@H](CSc3ccccc3)Nc3ccc(S(=O)(=O)NC(=O)c4ccc(N5CCC6(CC5)CC(N5CCC[C@H]5c5ccccc5C5CC5)C6)cc4)cc3S(=O)(=O)C(F)(F)F)CC2)c1] |
| I-32 | 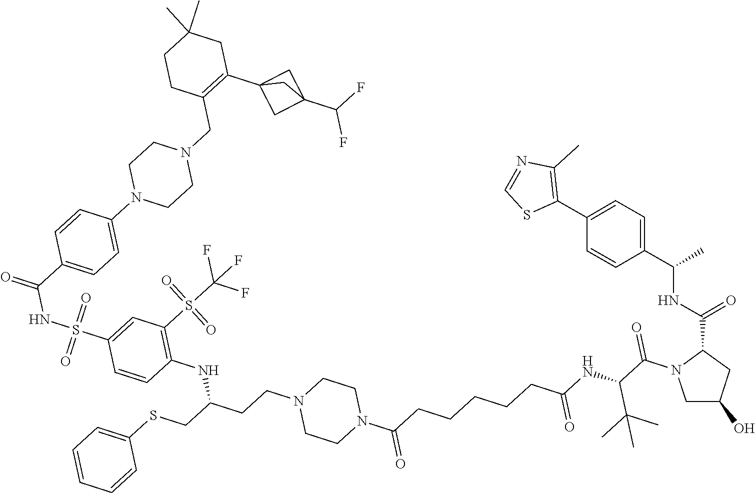 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(C78CC(C(F)F)(C7)C8)CC(C)(C)CC6)CC5)cc4)cc3S(=O)(=O)C(F)(F)F)CC2)C(C)(C)C)cc1] |
| I-33 | 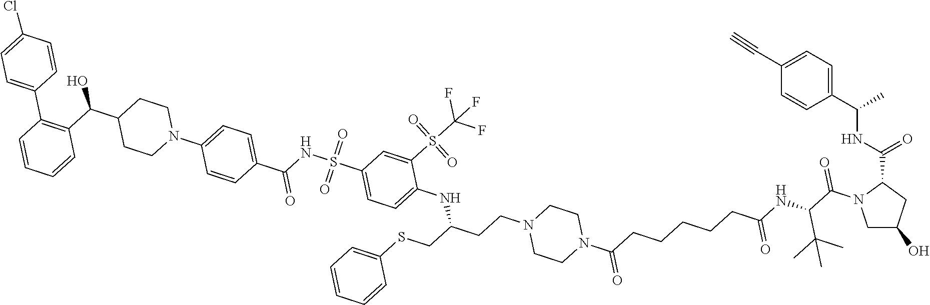 [SMILES: C#Cc1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCC([C@H](O)c6ccccc6-c6ccc(Cl)cc6)CC5)cc4)cc3S(=O)(=O)C(F)(F)F)CC2)C(C)(C)C)cc1] |
| I-34 | 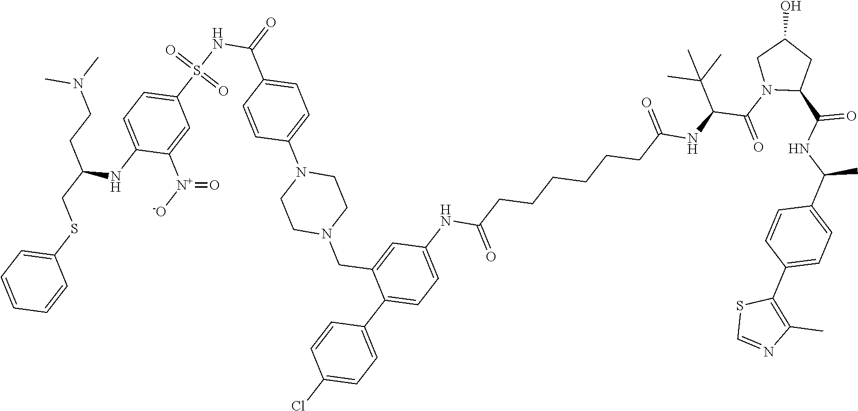 |
| I-35 | 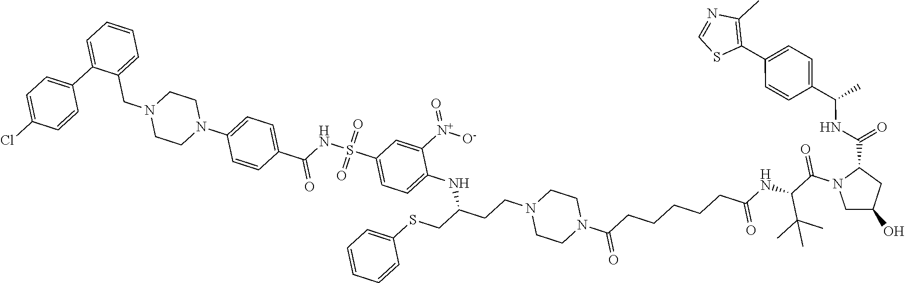 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(Cc6ccccc6-c6ccc(Cl)cc6)CC5)cc4)cc3[N+](=O)[O-])CC2)C(C)(C)C)cc1] |
| I-36 | 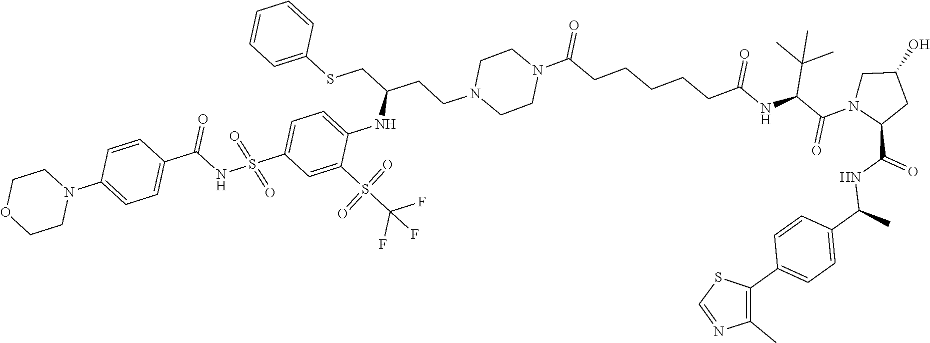 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCOCC5)cc4)cc3S(=O)(=O)C(F)(F)F)CC2)C(C)(C)C)cc1] |
| I-37 | 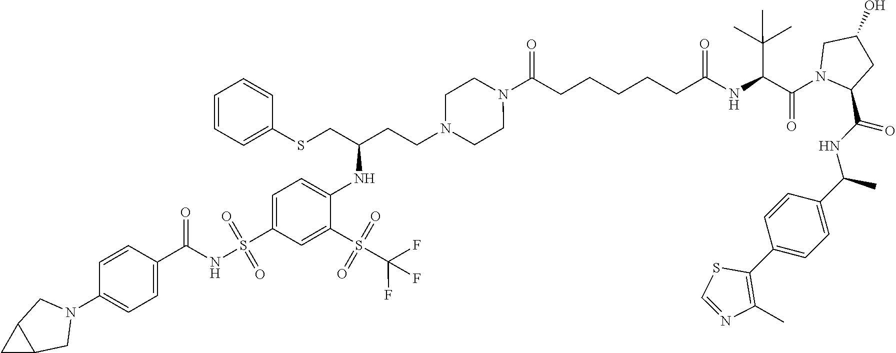 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CC6CC6C5)cc4)cc3S(=O)(=O)C(F)(F)F)CC2)C(C)(C)C)cc1] |
| I-38 | 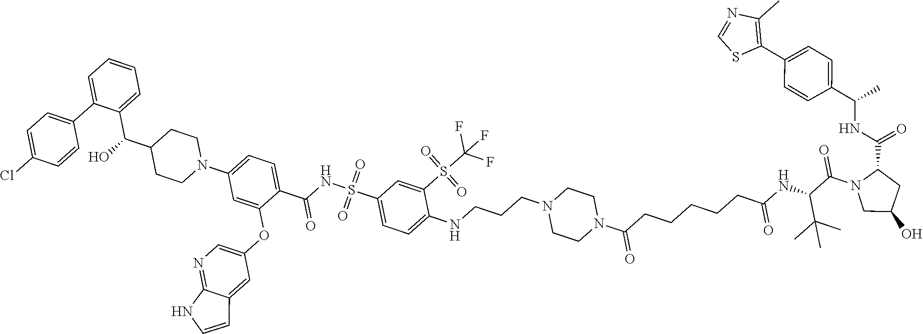 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCC([C@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-39 | 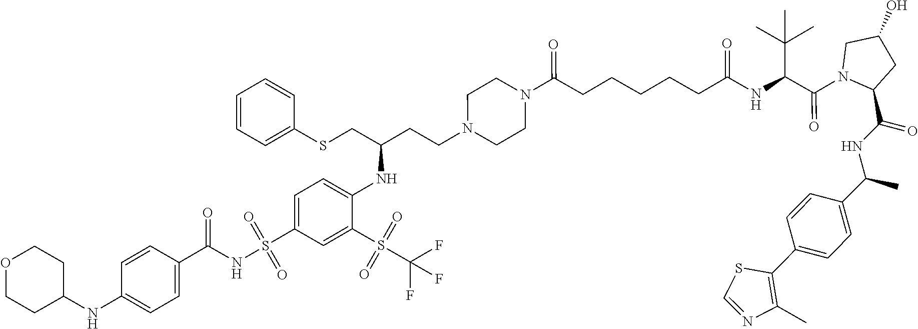 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(NC5CCOCC5)cc4)cc3S(=O)(=O)C(F)(F)F)CC2)C(C)(C)C)cc1] |
| I-40 | 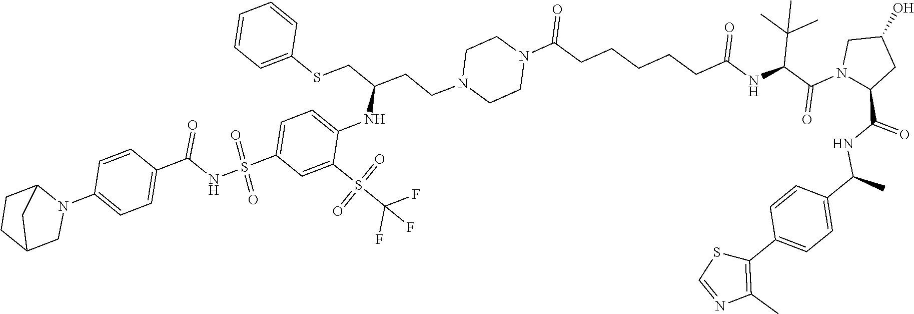 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CC6CCC5C6)cc4)cc3S(=O)(=O)C(F)(F)F)CC2)C(C)(C)C)cc1] |
| I-41 | 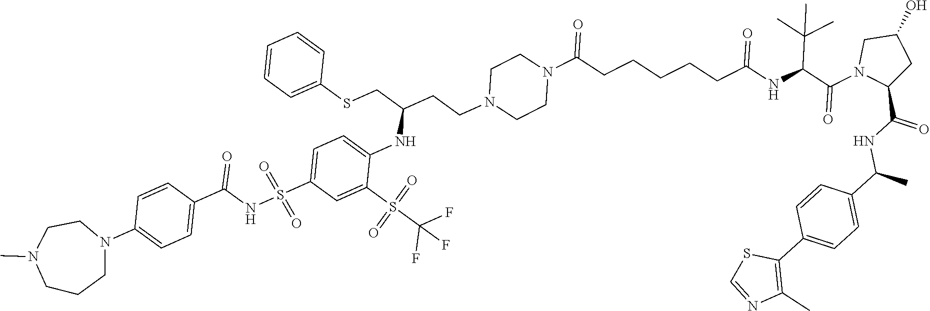 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCCN(C)CC5)cc4)cc3S(=O)(=O)C(F)(F)F)CC2)C(C)(C)C)cc1] |
| I-42 | 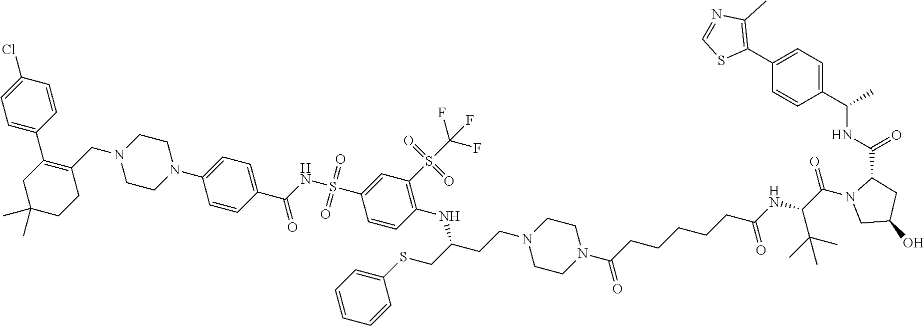 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CC(C)(C)CC6)CC5)cc4)cc3S(=O)(=O)C(F)(F)F)CC2)C(C)(C)C)cc1] |
| I-43 | 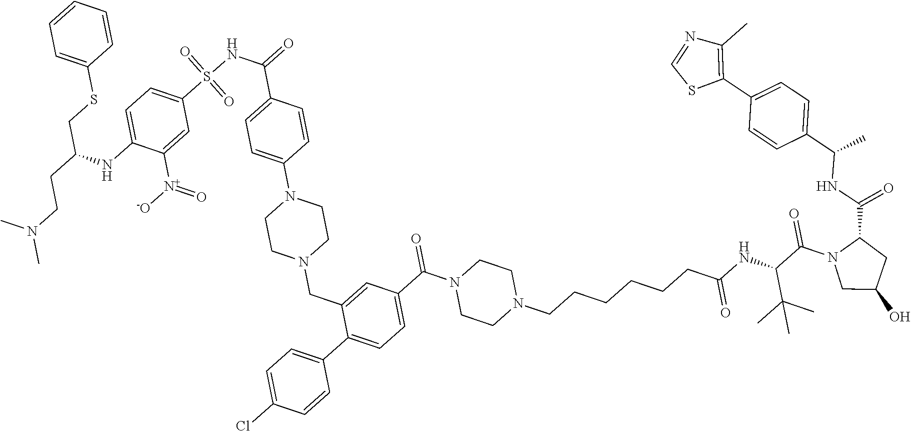 |
| I-44 | 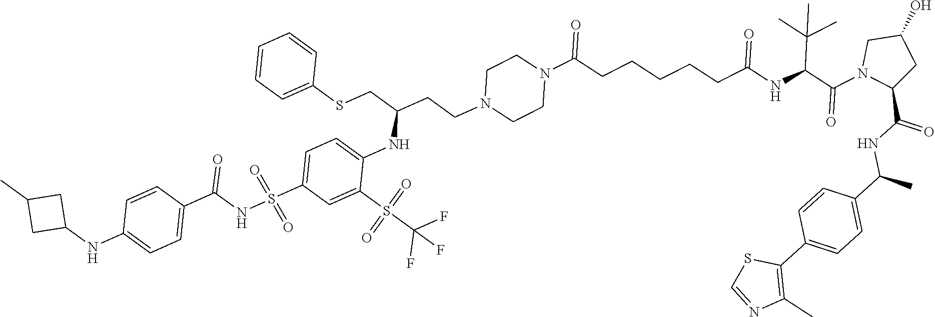 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(NC5CC(C)C5)cc4)cc3S(=O)(=O)C(F)(F)F)CC2)C(C)(C)C)cc1] |
| I-45 | 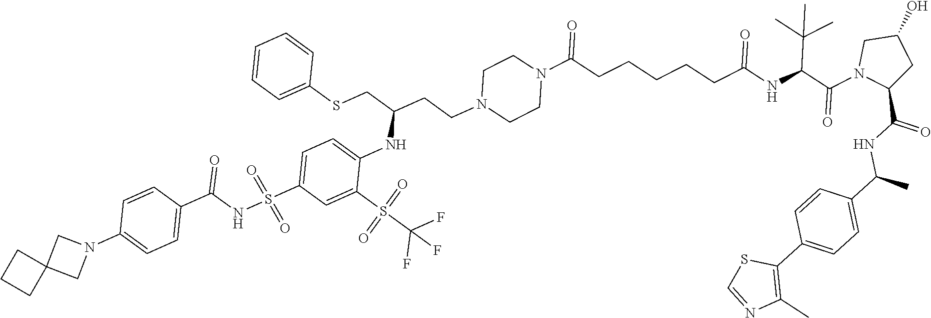 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CC6(CCC6)C5)cc4)cc3S(=O)(=O)C(F)(F)F)CC2)C(C)(C)C)cc1] |
| I-46 | 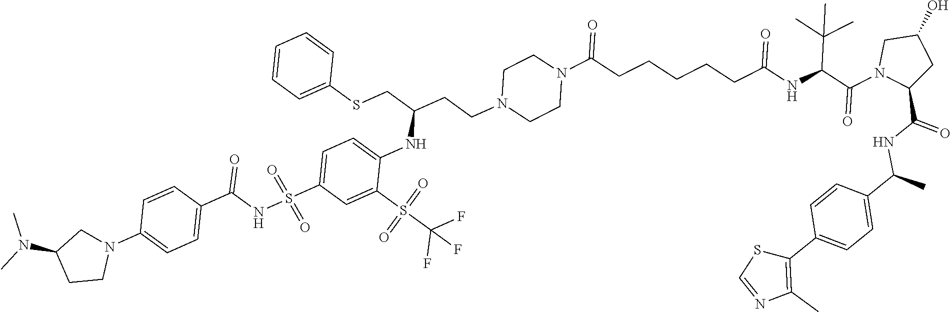 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CC[C@@H](N(C)C)C5)cc4)cc3S(=O)(=O)C(F)(F)F)CC2)C(C)(C)C)cc1] |
| I-47 | 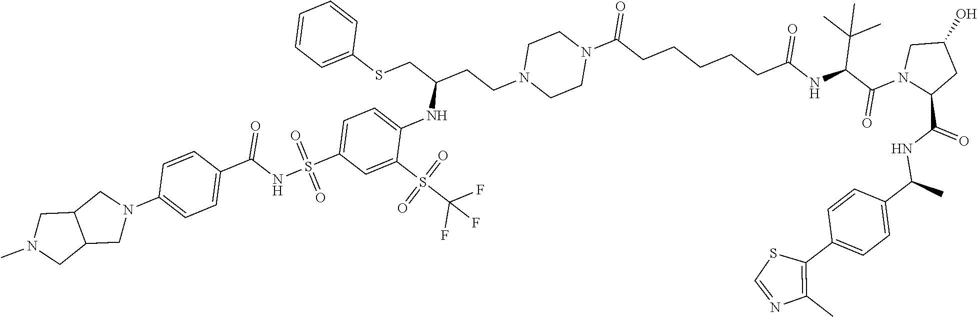 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CC6CN(C)CC6C5)cc4)cc3S(=O)(=O)C(F)(F)F)CC2)C(C)(C)C)cc1] |
| I-48 | 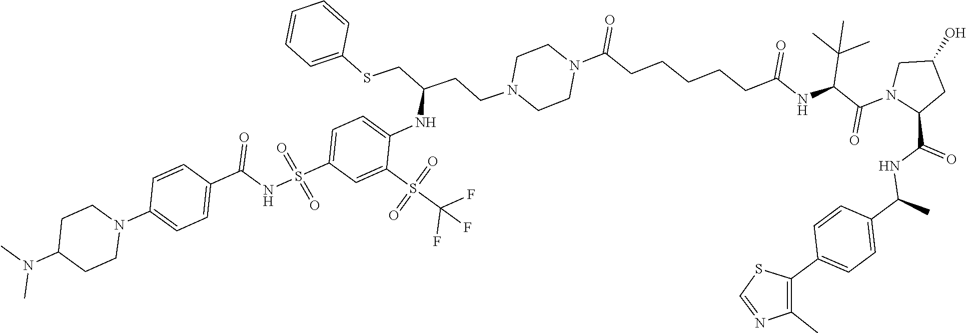 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCC(N(C)C)CC5)cc4)cc3S(=O)(=O)C(F)(F)F)CC2)C(C)(C)C)cc1] |
| I-49 | 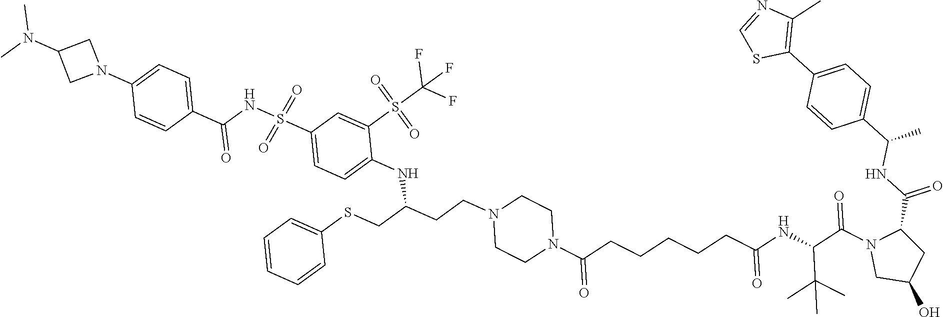 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CC(N(C)C)C5)cc4)cc3S(=O)(=O)C(F)(F)F)CC2)C(C)(C)C)cc1] |
| I-50 | 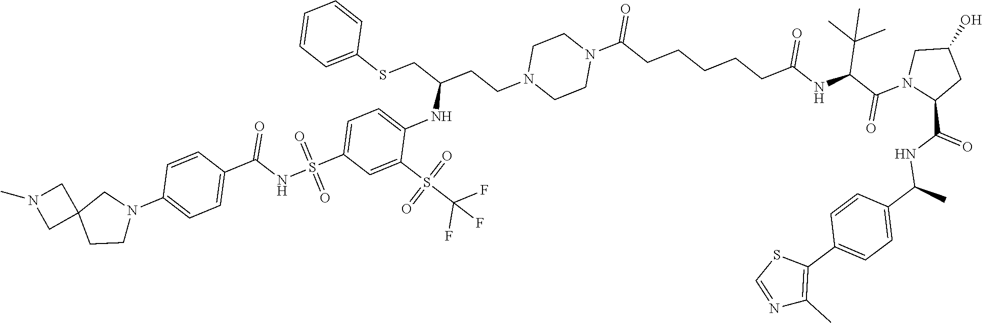 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCC6(CN(C)C6)C5)cc4)cc3S(=O)(=O)C(F)(F)F)CC2)C(C)(C)C)cc1] |
| I-51 | 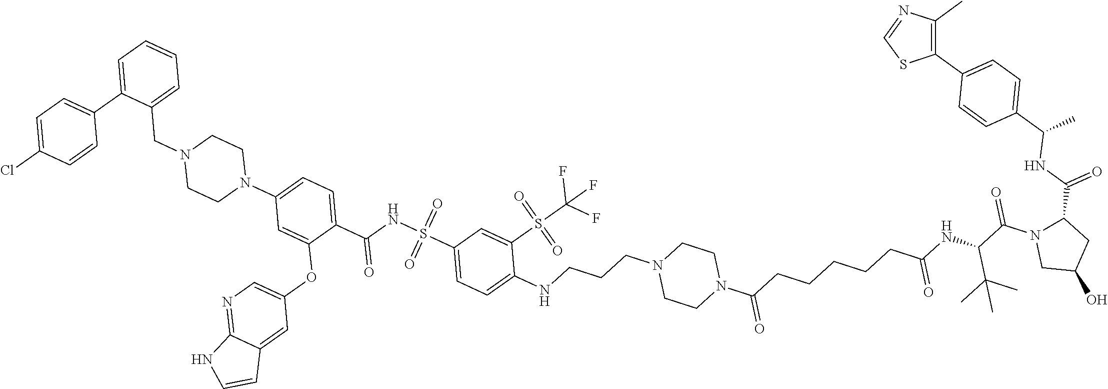 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(Cc6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-52 | 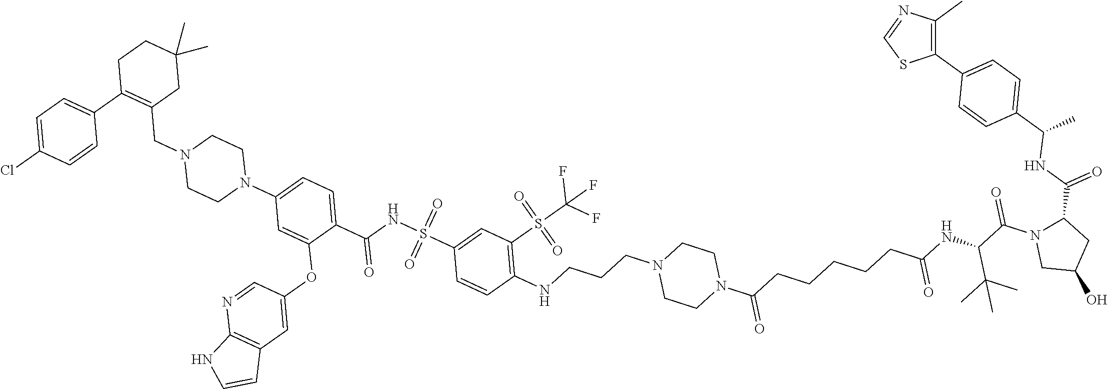 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CCC(C)(C)C6)CC5)cc4Oc4cnc5[nH]ccc5c4)cc3S(=O)(=O)C(F)(F)F)CC2)C(C)(C)C)cc1] |
| I-53 | 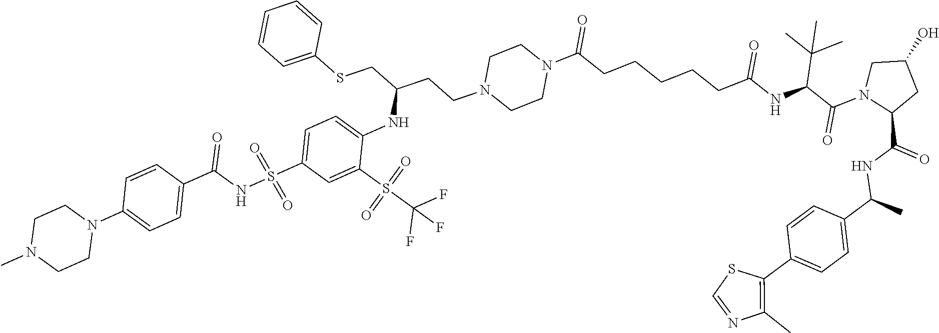 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(C)CC5)cc4)cc3S(=O)(=O)C(F)(F)F)CC2)C(C)(C)C)cc1] |
| I-54 | 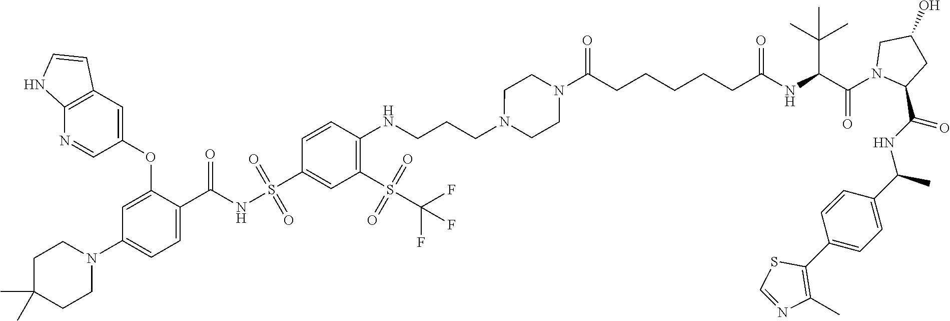 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCC(C)(C)CC5)cc4Oc4cnc5[nH]ccc5c4)cc3S(=O)(=O)C(F)(F)F)CC2)C(C)(C)C)cc1] |
| I-55 | 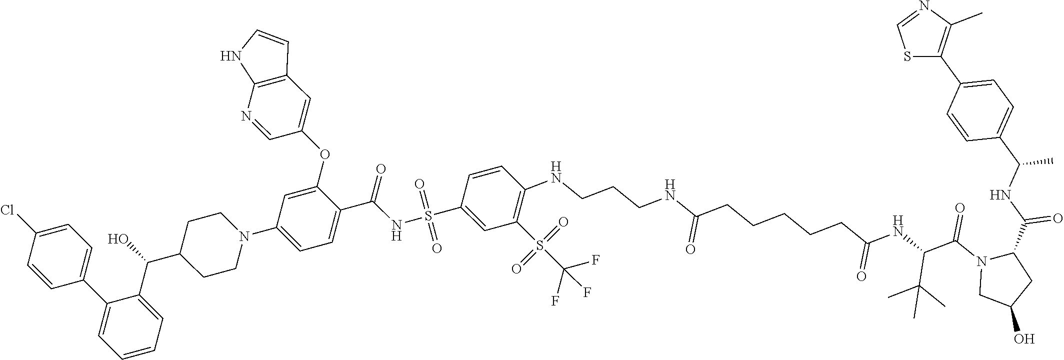 [SMILES: Cc1ncsc1-c1ccc([C@H](C)NC(=O)[C@@H]2C[C@@H](O)CN2C(=O)[C@@H](NC(=O)CCCCCC(=O)NCCCNc2ccc(S(=O)(=O)NC(=O)c3ccc(N4CCC([C@@H](O)c5ccccc5-c5ccc(Cl)cc5)CC4)cc3Oc3cnc4[nH]ccc4c3)cc2S(=O)(=O)C(F)(F)F)C(C)(C)C)cc1] |
| I-56 | 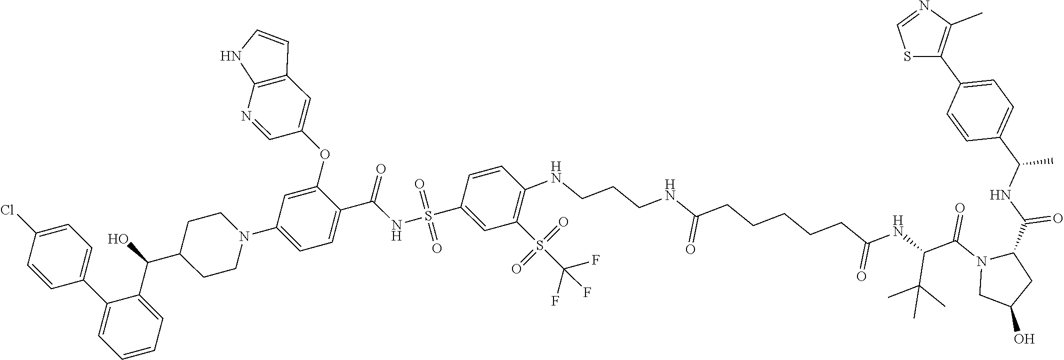 [SMILES: Cc1ncsc1-c1ccc([C@H](C)NC(=O)[C@@H]2C[C@@H](O)CN2C(=O)[C@@H](NC(=O)CCCCCC(=O)NCCCNc2ccc(S(=O)(=O)NC(=O)c3ccc(N4CCC([C@H](O)c5ccccc5-c5ccc(Cl)cc5)CC4)cc3Oc3cnc4[nH]ccc4c3)cc2S(=O)(=O)C(F)(F)F)C(C)(C)C)cc1] |
| I-57 | 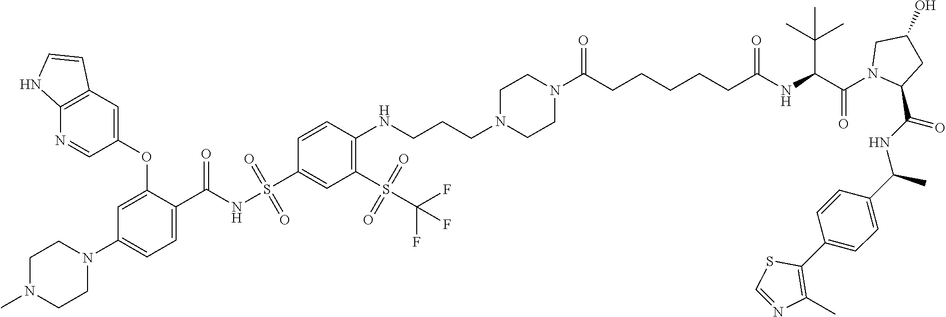 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(C)CC5)cc4Oc4cnc5[nH]ccc5c4)cc3S(=O)(=O)C(F)(F)F)CC2)C(C)(C)C)cc1] |
| I-58 | 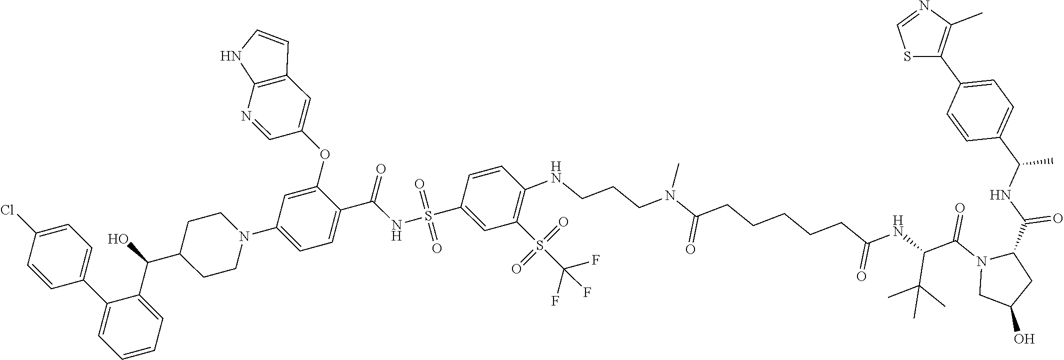 [SMILES: Cc1ncsc1-c1ccc([C@H](C)NC(=O)[C@@H]2C[C@@H](O)CN2C(=O)[C@@H](NC(=O)CCCCCC(=O)N(C)CCCNc2ccc(S(=O)(=O)NC(=O)c3ccc(N4CCC([C@H](O)c5ccccc5-c5ccc(Cl)cc5)CC4)cc3Oc3cnc4[nH]ccc4c3)cc2S(=O)(=O)C(F)(F)F)C(C)(C)C)cc1] |
| I-59 | 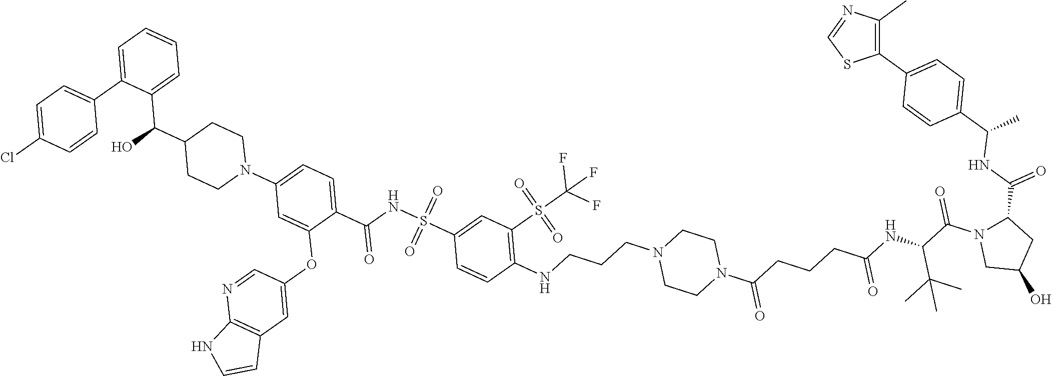 [SMILES: Cc1ncsc1-c1ccc([C@H](C)NC(=O)[C@@H]2C[C@@H](O)CN2C(=O)[C@@H](NC(=O)CCCC(=O)N2CCN(CCCNc3ccc(S(=O)(=O)NC(=O)c4ccc(N5CCC([C@@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-60 | 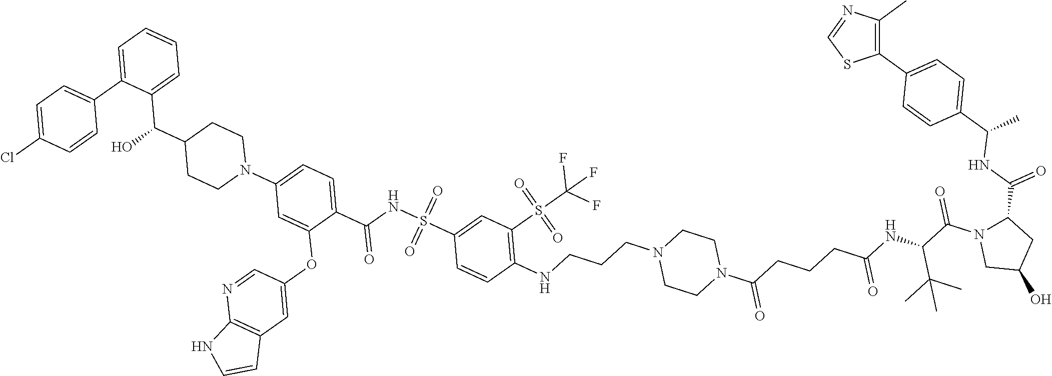 [SMILES: Cc1ncsc1-c1ccc([C@H](C)NC(=O)[C@@H]2C[C@@H](O)CN2C(=O)[C@@H](NC(=O)CCCC(=O)N2CCN(CCCNc3ccc(S(=O)(=O)NC(=O)c4ccc(N5CCC([C@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-61 | 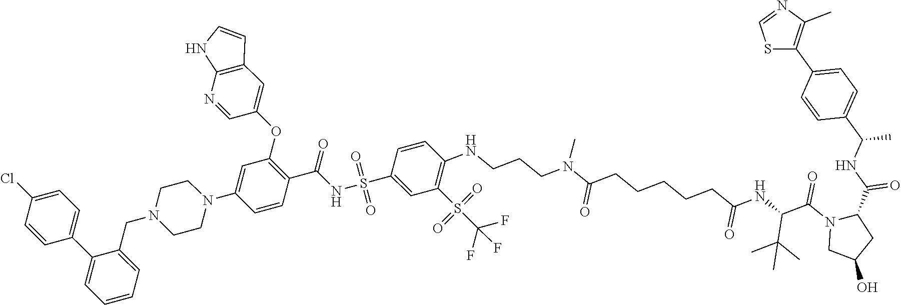 [SMILES: Cc1ncsc1-c1ccc([C@H](C)NC(=O)[C@@H]2C[C@@H](O)CN2C(=O)[C@@H](NC(=O)CCCCCC(=O)N(C)CCCNc2ccc(S(=O)(=O)NC(=O)c3ccc(N4CCN(Cc5ccccc5-c5ccc(Cl)cc5)CC4)cc3Oc3cnc4[nH]ccc4c3)cc2S(=O)(=O)C(F)(F)F)C(C)(C)C)cc1] |
| I-62 | 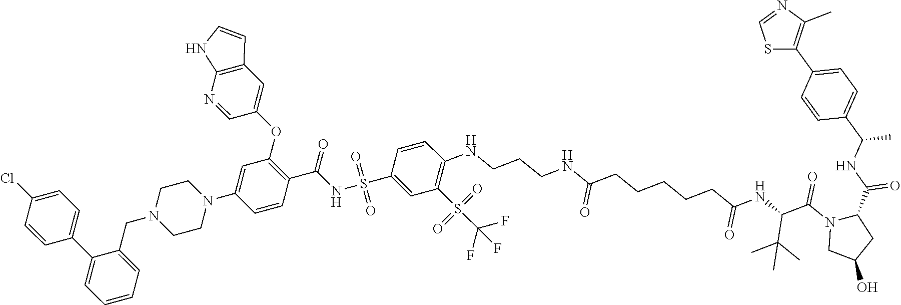 [SMILES: Cc1ncsc1-c1ccc([C@H](C)NC(=O)[C@@H]2C[C@@H](O)CN2C(=O)[C@@H](NC(=O)CCCCCC(=O)NCCCNc2ccc(S(=O)(=O)NC(=O)c3ccc(N4CCN(Cc5ccccc5-c5ccc(Cl)cc5)CC4)cc3Oc3cnc4[nH]ccc4c3)cc2S(=O)(=O)C(F)(F)F)C(C)(C)C)cc1] |
| I-63 | 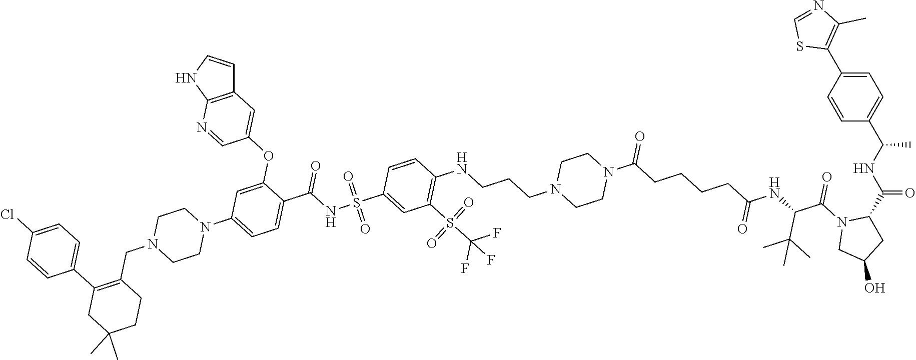 [SMILES: Cc1ncsc1-c1ccc([C@H](C)NC(=O)[C@@H]2C[C@@H](O)CN2C(=O)[C@@H](NC(=O)CCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-64 | 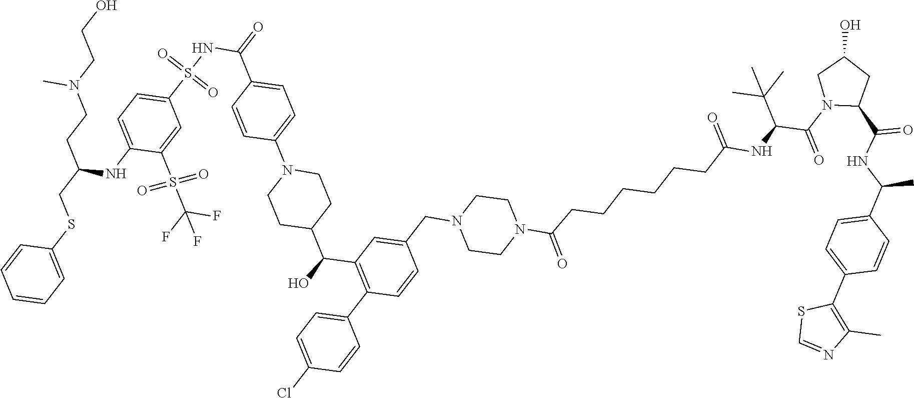 [SMILES: Cc1ncsc1-c1ccc([C@H](C)NC(=O)[C@@H]2C[C@@H](O)CN2C(=O)[C@@H](NC(=O)CCCCCCC(=O)N2CCN(Cc3ccc(-c4ccc(Cl)cc4)c([C@@H](O)C4CCN(c5ccc(C(=O)NS(=O)(=O)c6ccc(N[C@H](CCN(C)CCO)CSc7ccccc7)c(S(=O)(=O)C(F)(F)F)c6)cc5)CC4)c3)CC2)C(C)(C)C)cc1] |
| I-65 | 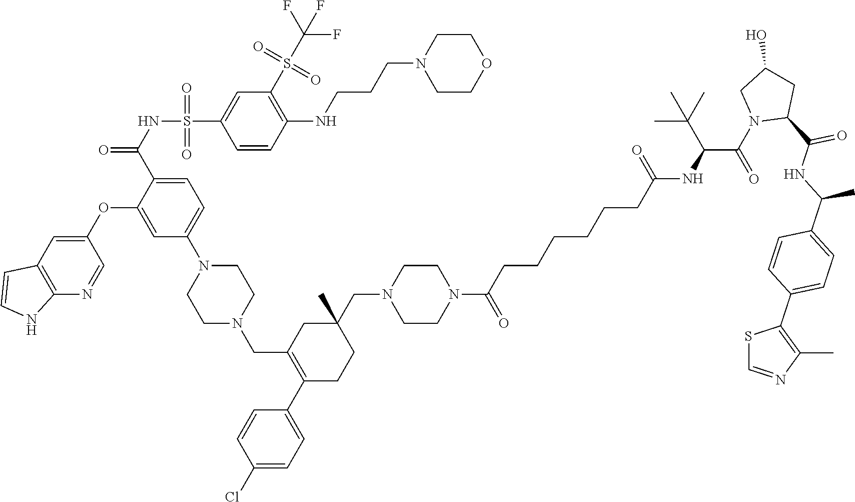 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-66 | 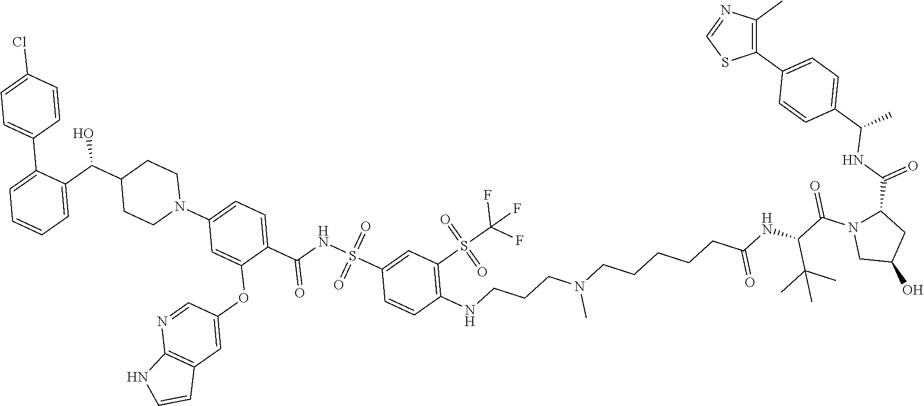 [SMILES: Cc1ncsc1-c1ccc([C@H](C)NC(=O)[C@@H]2C[C@@H](O)CN2C(=O)[C@@H](NC(=O)CCCCCN(C)CCCNc2ccc(S(=O)(=O)NC(=O)c3ccc(N4CCC([C@@H](O)c5ccccc5-c5ccc(Cl)cc5)CC4)cc3Oc3cnc4[nH]ccc4c3)cc2S(=O)(=O)C(F)(F)F)C(C)(C)C)cc1] |
| I-67 | 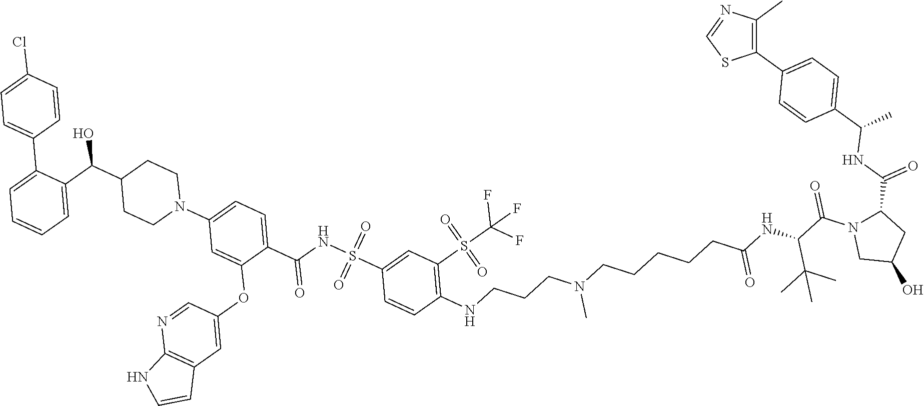 [SMILES: Cc1ncsc1-c1ccc([C@H](C)NC(=O)[C@@H]2C[C@@H](O)CN2C(=O)[C@@H](NC(=O)CCCCCN(C)CCCNc2ccc(S(=O)(=O)NC(=O)c3ccc(N4CCC([C@H](O)c5ccccc5-c5ccc(Cl)cc5)CC4)cc3Oc3cnc4[nH]ccc4c3)cc2S(=O)(=O)C(F)(F)F)C(C)(C)C)cc1] |
| I-68 | 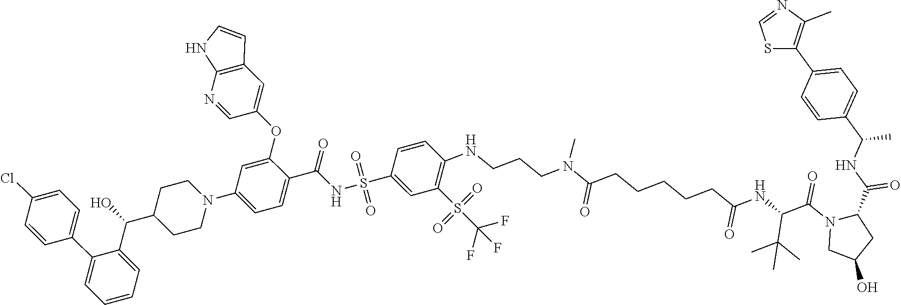 [SMILES: Cc1ncsc1-c1ccc([C@H](C)NC(=O)[C@@H]2C[C@@H](O)CN2C(=O)[C@@H](NC(=O)CCCCCC(=O)N(C)CCCNc2ccc(S(=O)(=O)NC(=O)c3ccc(N4CCC([C@@H](O)c5ccccc5-c5ccc(Cl)cc5)CC4)cc3Oc3cnc4[nH]ccc4c3)cc2S(=O)(=O)C(F)(F)F)C(C)(C)C)cc1] |
| I-69 | 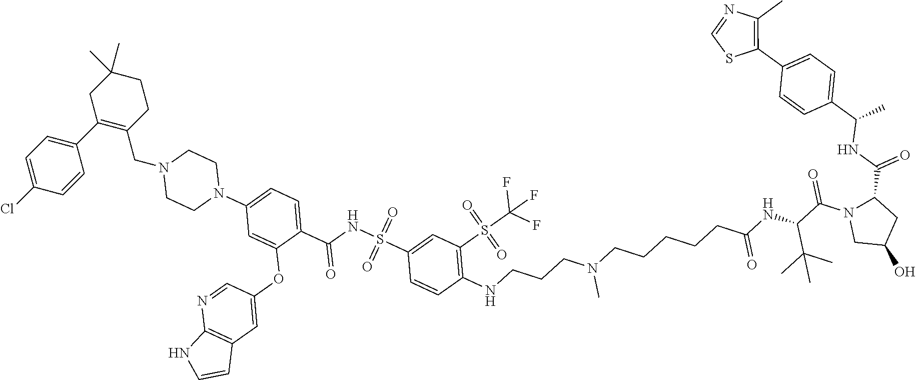 [SMILES: Cc1ncsc1-c1ccc([C@H](C)NC(=O)[C@@H]2C[C@@H](O)CN2C(=O)[C@@H](NC(=O)CCCCCN(C)CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-70 | 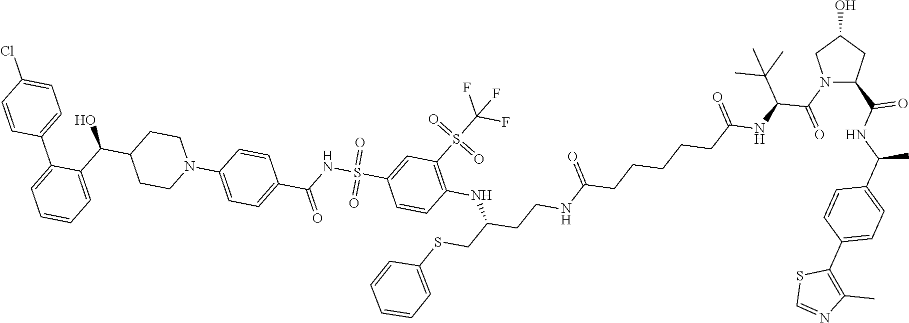 [SMILES: Cc1ncsc1-c1ccc([C@H](C)NC(=O)[C@@H]2C[C@@H](O)CN2C(=O)[C@@H](NC(=O)CCCCCC(=O)NCC[C@H](CSc2ccccc2)Nc2ccc(S(=O)(=O)NC(=O)c3ccc(N4CCC([C@H](O)c5ccccc5-c5ccc(Cl)cc5)CC4)cc3)cc2S(=O)(=O)C(F)(F)F)C(C)(C)C)cc1] |
| I-71 | 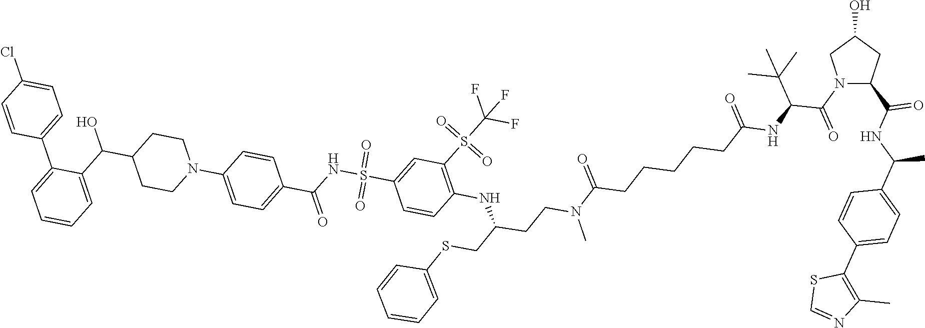 [SMILES: Cc1ncsc1-c1ccc([C@H](C)NC(=O)[C@@H]2C[C@@H](O)CN2C(=O)[C@@H](NC(=O)CCCCCC(=O)N(C)CC[C@H](CSc2ccccc2)Nc2ccc(S(=O)(=O)NC(=O)c3ccc(N4CCC(C(O)c5ccccc5-c5ccc(Cl)cc5)CC4)cc3)cc2S(=O)(=O)C(F)(F)F)C(C)(C)C)cc1] |
| I-72 | 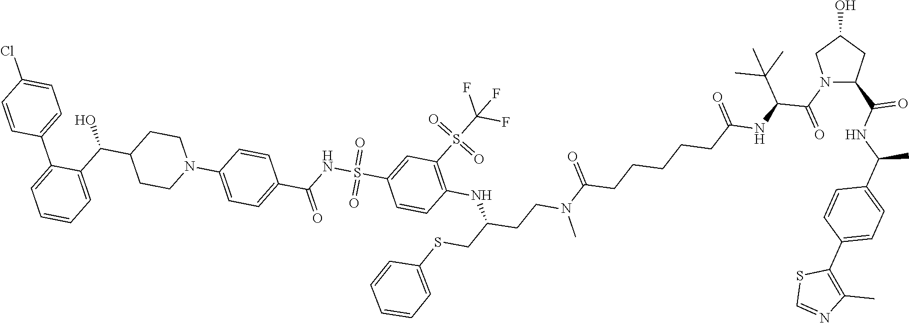 [SMILES: Cc1ncsc1-c1ccc([C@H](C)NC(=O)[C@@H]2C[C@@H](O)CN2C(=O)[C@@H](NC(=O)CCCCCC(=O)N(C)CC[C@H](CSc2ccccc2)Nc2ccc(S(=O)(=O)NC(=O)c3ccc(N4CCC([C@@H](O)c5ccccc5-c5ccc(Cl)cc5)CC4)cc3)cc2S(=O)(=O)C(F)(F)F)C(C)(C)C)cc1] |
| I-73 | 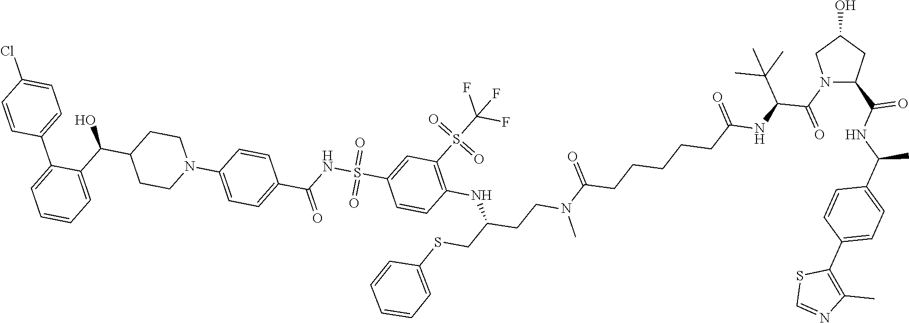 [SMILES: Cc1ncsc1-c1ccc([C@H](C)NC(=O)[C@@H]2C[C@@H](O)CN2C(=O)[C@@H](NC(=O)CCCCCC(=O)N(C)CC[C@H](CSc2ccccc2)Nc2ccc(S(=O)(=O)NC(=O)c3ccc(N4CCC([C@H](O)c5ccccc5-c5ccc(Cl)cc5)CC4)cc3)cc2S(=O)(=O)C(F)(F)F)C(C)(C)C)cc1] |
| I-74 | 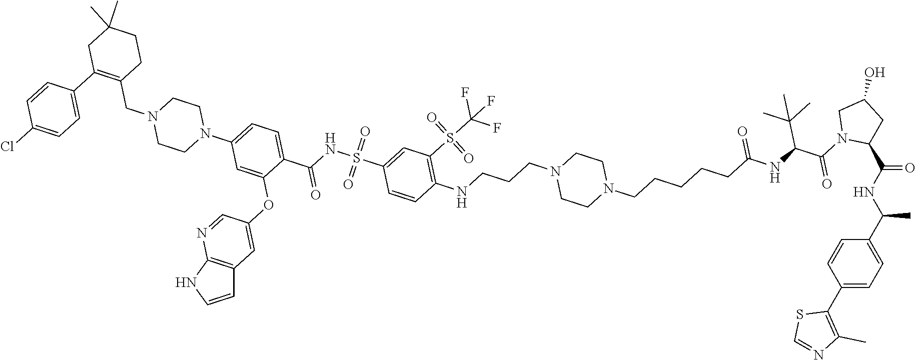 [SMILES: Cc1ncsc1-c1ccc([C@H](C)NC(=O)[C@@H]2C[C@@H](O)CN2C(=O)[C@@H](NC(=O)CCCCCN2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-75 | 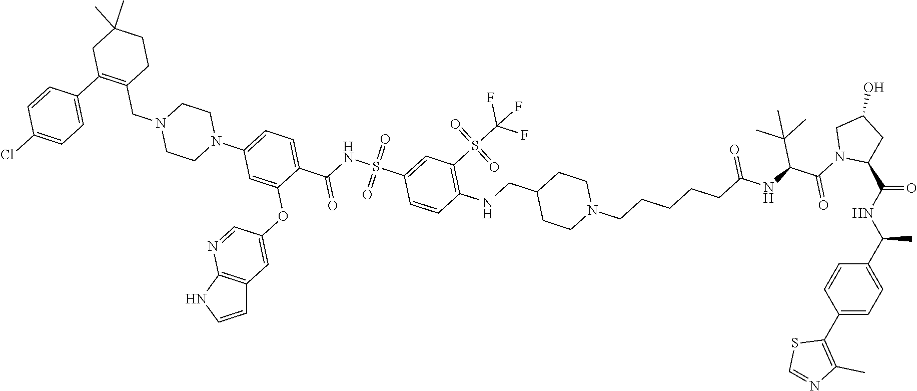 [SMILES: Cc1ncsc1-c1ccc([C@H](C)NC(=O)[C@@H]2C[C@@H](O)CN2C(=O)[C@@H](NC(=O)CCCCCN2CCC(CNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-76 | 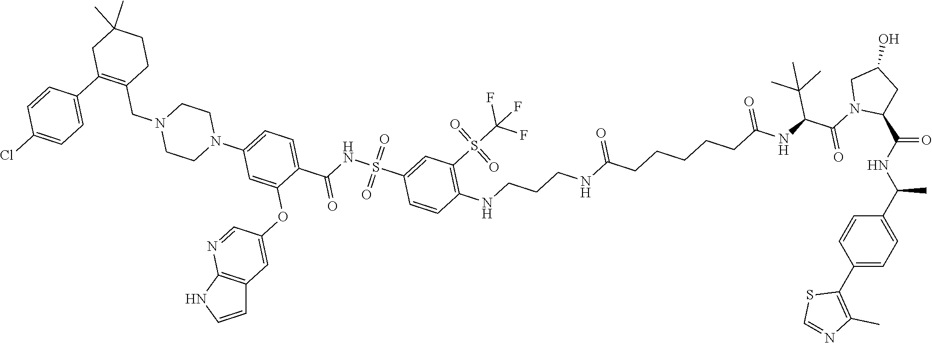 [SMILES: Cc1ncsc1-c1ccc([C@H](C)NC(=O)[C@@H]2C[C@@H](O)CN2C(=O)[C@@H](NC(=O)CCCCCC(=O)NCCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-77 | 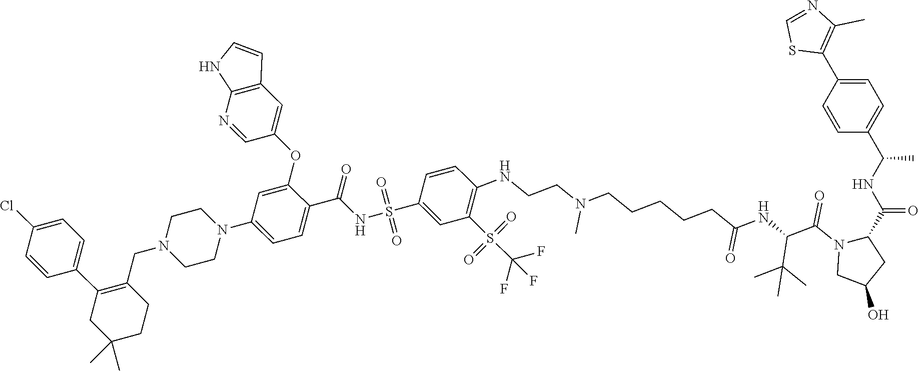 [SMILES: Cc1ncsc1-c1ccc([C@H](C)NC(=O)[C@@H]2C[C@@H](O)CN2C(=O)[C@@H](NC(=O)CCCCCN(C)CCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-78 | 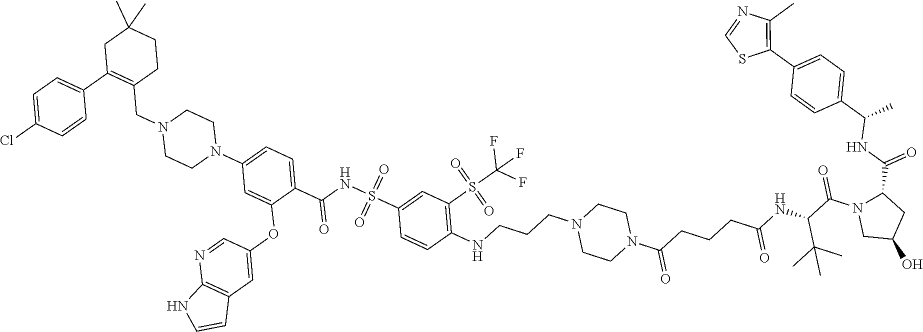 [SMILES: Cc1ncsc1-c1ccc([C@H](C)NC(=O)[C@@H]2C[C@@H](O)CN2C(=O)[C@@H](NC(=O)CCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-79 | 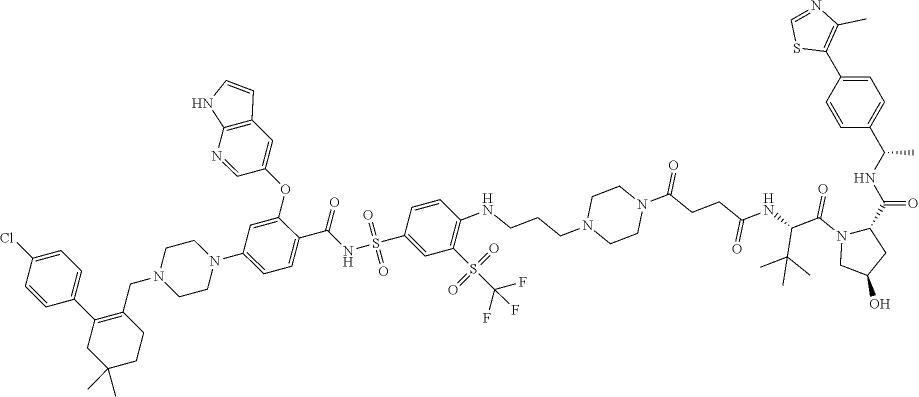 [SMILES: Cc1ncsc1-c1ccc([C@H](C)NC(=O)[C@@H]2C[C@@H](O)CN2C(=O)[C@@H](NC(=O)CCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-80 | 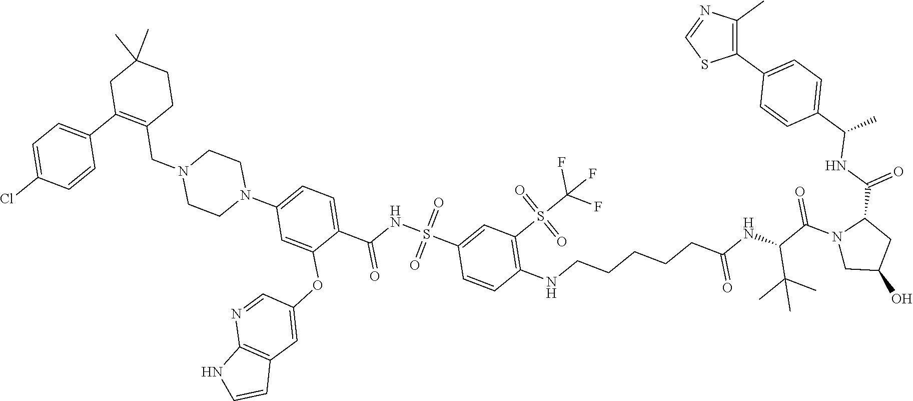 [SMILES: Cc1ncsc1-c1ccc([C@H](C)NC(=O)[C@@H]2C[C@@H](O)CN2C(=O)[C@@H](NC(=O)CCCCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-81 | 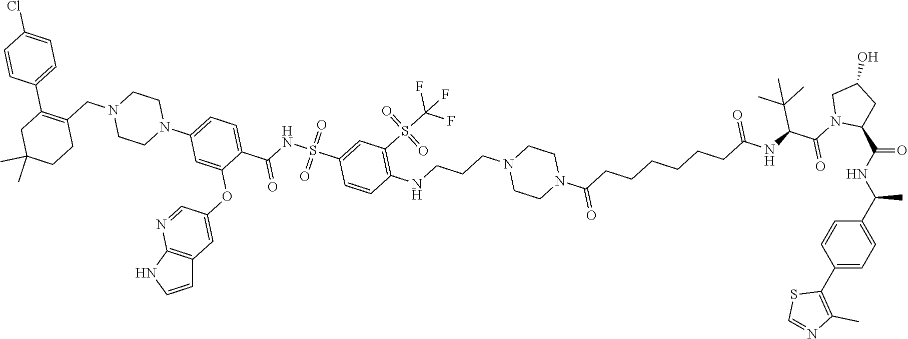 [SMILES: Cc1ncsc1-c1ccc([C@H](C)NC(=O)[C@@H]2C[C@@H](O)CN2C(=O)[C@@H](NC(=O)CCCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-82 | 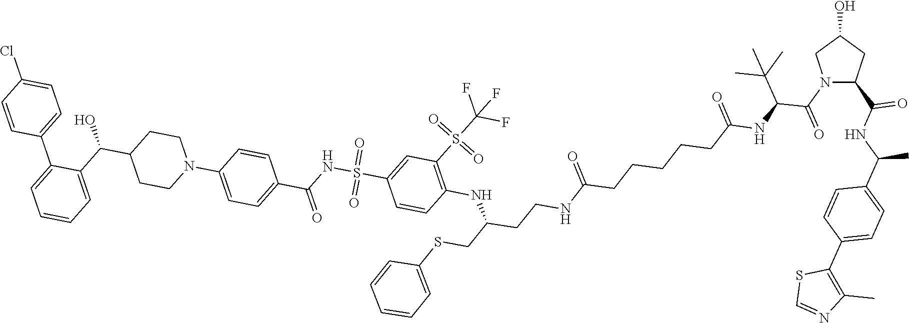 [SMILES: Cc1ncsc1-c1ccc([C@H](C)NC(=O)[C@@H]2C[C@@H](O)CN2C(=O)[C@@H](NC(=O)CCCCCC(=O)NCC[C@H](CSc2ccccc2)Nc2ccc(S(=O)(=O)NC(=O)c3ccc(N4CCC([C@@H](O)c5ccccc5-c5ccc(Cl)cc5)CC4)cc3)cc2S(=O)(=O)C(F)(F)F)C(C)(C)C)cc1] |
| I-83 | 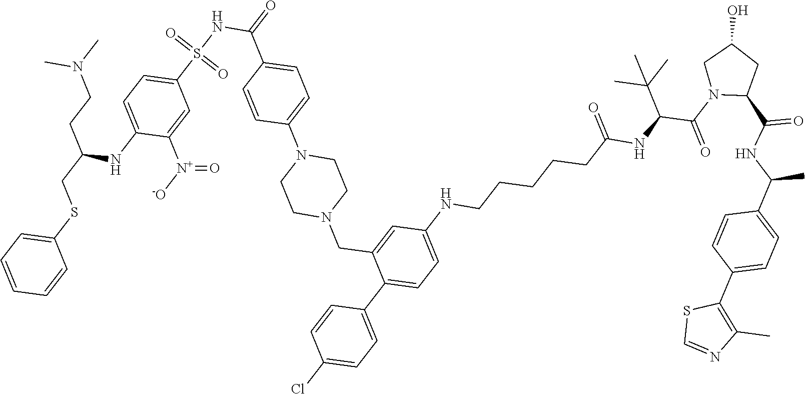 |
| I-84 | 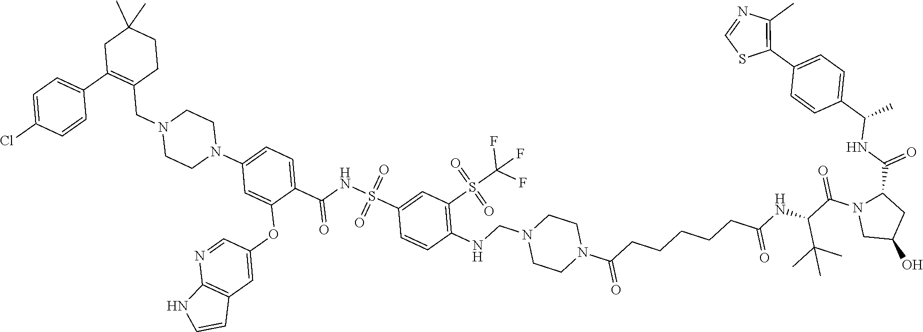 [SMILES: Cc1ncsc1-c1ccc([C@H](C)NC(=O)[C@@H]2C[C@@H](O)CN2C(=O)[C@@H](NC(=O)CCCCCC(=O)N2CCN(CNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-85 | 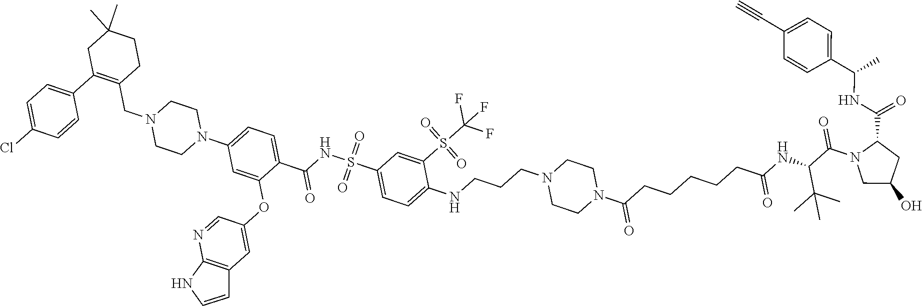 [SMILES: C#Cc1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-86 | 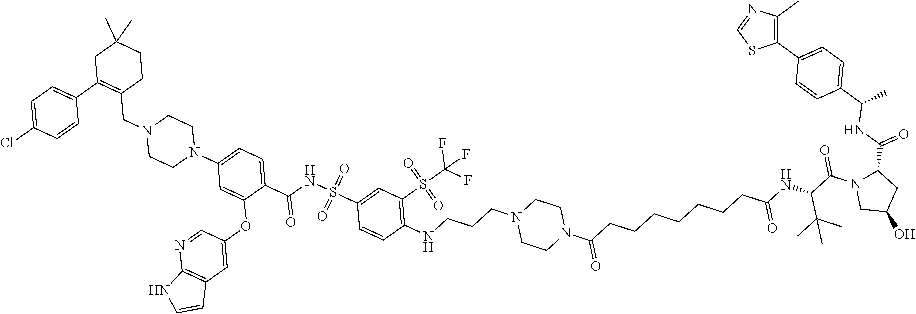 [SMILES: Cc1ncsc1-c1ccc([C@H](C)NC(=O)[C@@H]2C[C@@H](O)CN2C(=O)[C@@H](NC(=O)CCCCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-87 | 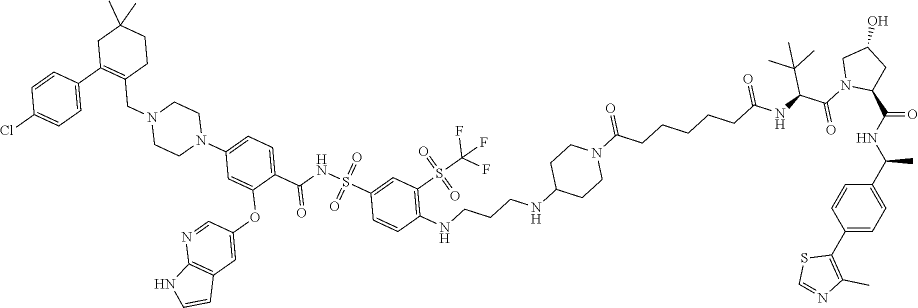 [SMILES: Cc1ncsc1-c1ccc([C@H](C)NC(=O)[C@@H]2C[C@@H](O)CN2C(=O)[C@@H](NC(=O)CCCCCC(=O)N2CCC(NCCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-88 | 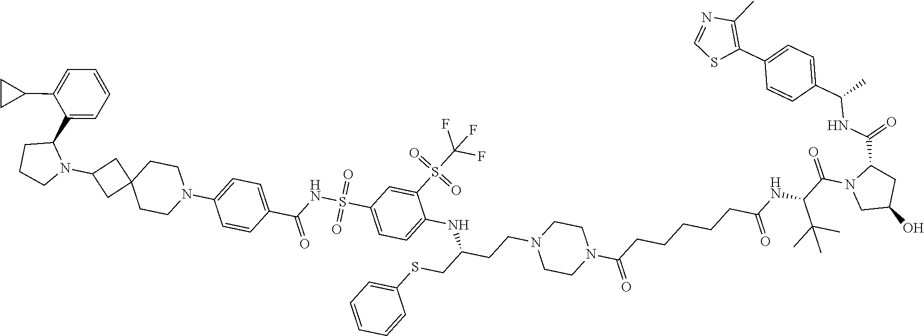 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCC6(CC5)CC(N5CCC[C@H]5c5ccccc5C5CC5)C6)cc4)cc3S(=O)(=O)C(F)(F)F)CC2)C(C)(C)C)cc1] |
| I-89 | 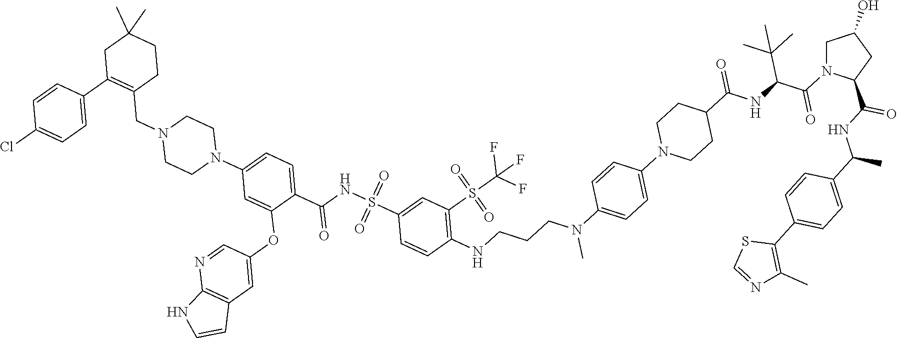 [SMILES: Cc1ncsc1-c1ccc([C@H](C)NC(=O)[C@@H]2C[C@@H](O)CN2C(=O)[C@@H](NC(=O)C2CCN(c3ccc(N(C)CCCNc4ccc(S(=O)(=O)NC(=O)c5ccc(N6CCN(CC7=C(c8ccc(Cl)cc8)CC(C)(C)CC7)CC6)cc5Oc5cnc6[nH]ccc6c5)cc4S(=O)(=O)C(F)(F)F)cc3)CC2)C(C)(C)C)cc1] |
| I-90 | 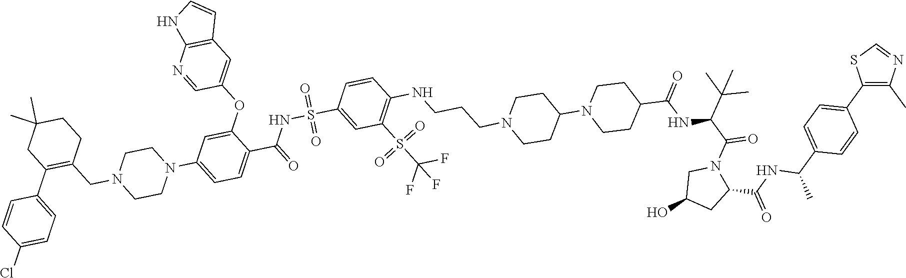 [SMILES: Cc1ncsc1-c1ccc([C@H](C)NC(=O)[C@@H]2C[C@@H](O)CN2C(=O)[C@@H](NC(=O)C2CCN(C3CCN(CCCNc4ccc(S(=O)(=O)NC(=O)c5ccc(N6CCN(CC7=C(c8ccc(Cl)cc8)CC(C)(C)CC7)CC6)cc5Oc5cnc6[nH]ccc6c5)cc4S(=O)(=O)C(F)(F)F)CC3)CC2)C(C)(C)C)cc1] |
| I-91 | 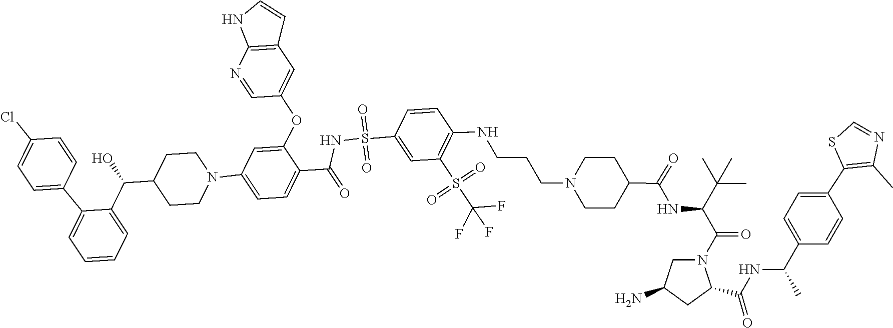 [SMILES: Cc1ncsc1-c1ccc([C@H](C)NC(=O)[C@@H]2C[C@@H](N)CN2C(=O)[C@@H](NC(=O)C2CCN(CCCNc3ccc(S(=O)(=O)NC(=O)c4ccc(N5CCC([C@@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-92 | 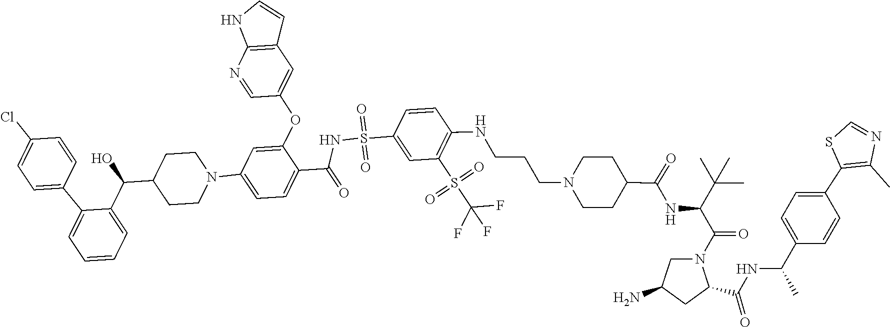 [SMILES: Cc1ncsc1-c1ccc([C@H](C)NC(=O)[C@@H]2C[C@@H](N)CN2C(=O)[C@@H](NC(=O)C2CCN(CCCNc3ccc(S(=O)(=O)NC(=O)c4ccc(N5CCC([C@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-93 | 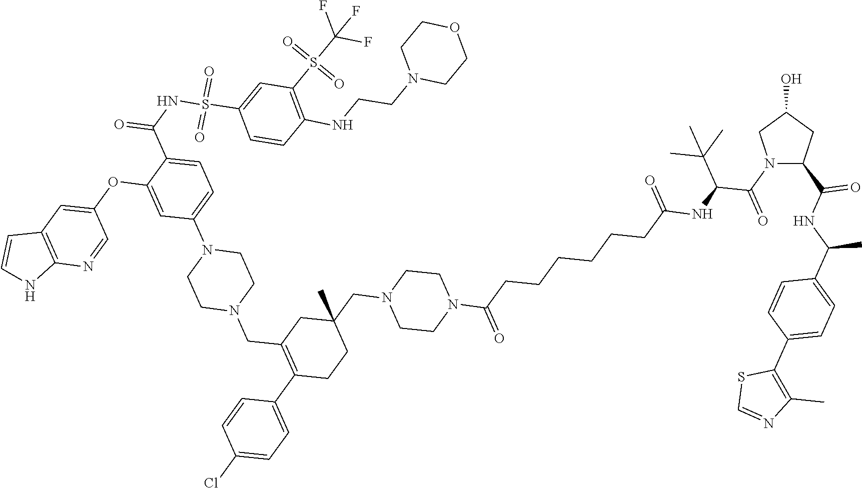 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NCCN7CCOCC7)c(S(=O)(=O)C(F)(F)F)c6)c(Oc6cnc7[nH]ccc7c6)c5)CC4)C3)CC2)C(C)(C)C)cc1] |
| I-94 | 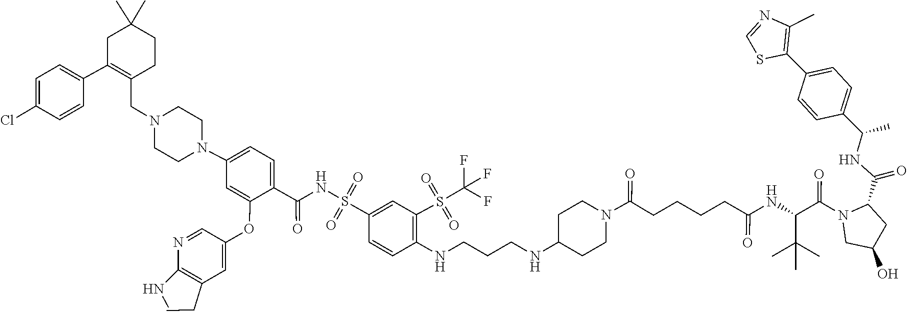 [SMILES: CCc1cc(Oc2cc(N3CCN(CC4=C(c5ccc(Cl)cc5)CC(C)(C)CC4)CC3)ccc2C(=O)NS(=O)(=O)c2ccc(NCCCNC3CCN(C(=O)CCCCC(=O)N[C@H](C(=O)N4C[C@H](O)C[C@H]4C(=O)N[C@@H](C)c4ccc(-c5scnc5C)cc4)C(C)(C)C)CC3)c(S(=O)(=O)C(F)(F)F)c2)cnc1NC] |
| I-95 | 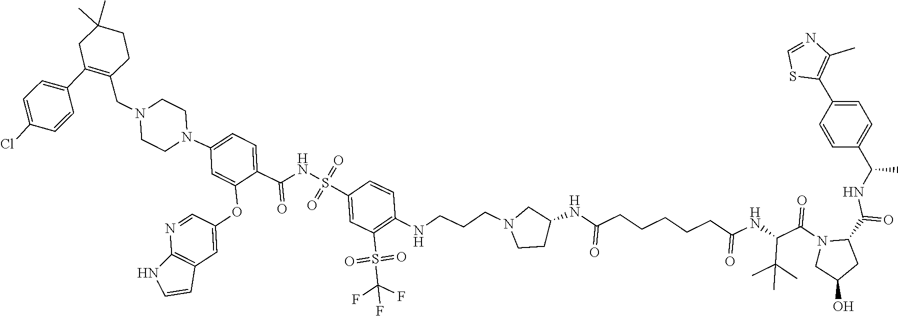 [SMILES: Cc1ncsc1-c1ccc([C@H](C)NC(=O)[C@@H]2C[C@@H](O)CN2C(=O)[C@@H](NC(=O)CCCCCC(=O)N[C@@H]2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)C2)C(C)(C)C)cc1] |
| I-96 | 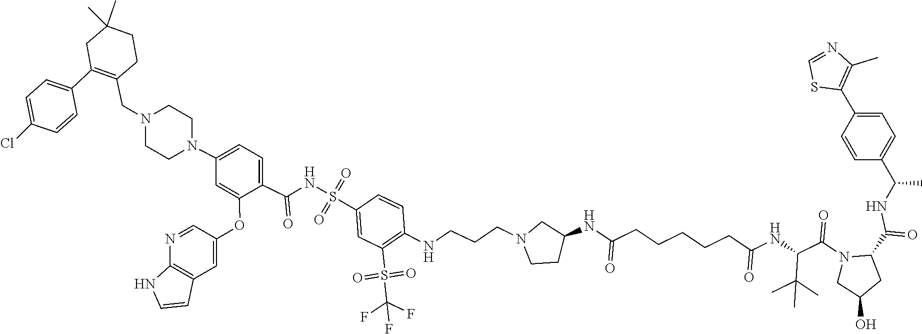 [SMILES: Cc1ncsc1-c1ccc([C@H](C)NC(=O)[C@@H]2C[C@@H](O)CN2C(=O)[C@@H](NC(=O)CCCCCC(=O)N[C@H]2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)C2)C(C)(C)C)cc1] |
| I-97 | 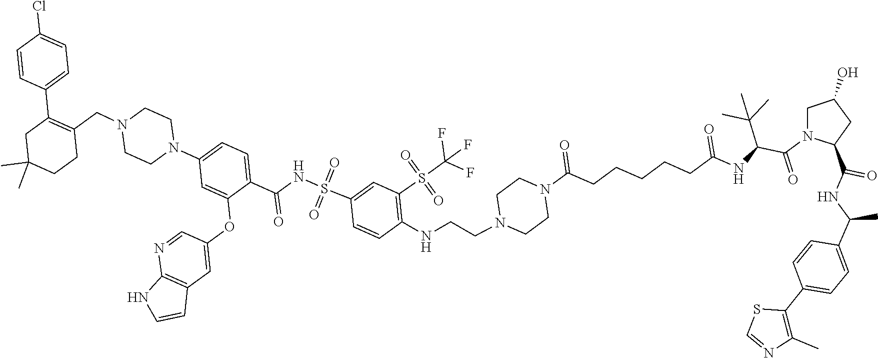 [SMILES: Cc1ncsc1-c1ccc([C@H](C)NC(=O)[C@@H]2C[C@@H](O)CN2C(=O)[C@@H](NC(=O)CCCCCC(=O)N2CCN(CCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-98 | 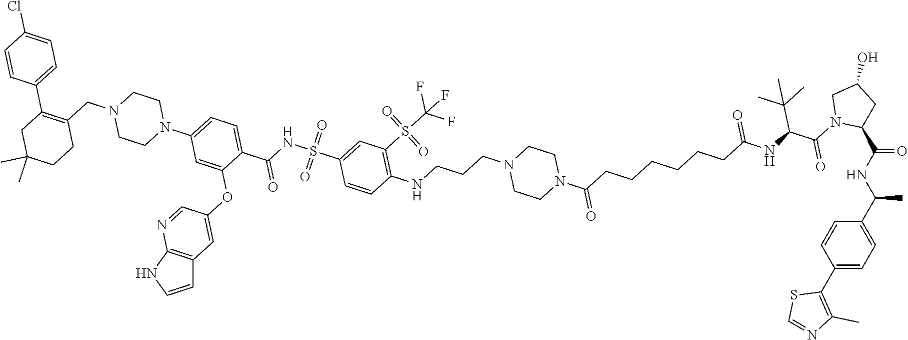 [SMILES: Cc1ncsc1-c1ccc([C@H](C)NC(=O)[C@@H]2C[C@@H](O)CN2C(=O)[C@@H](NC(=O)CCCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-99 | 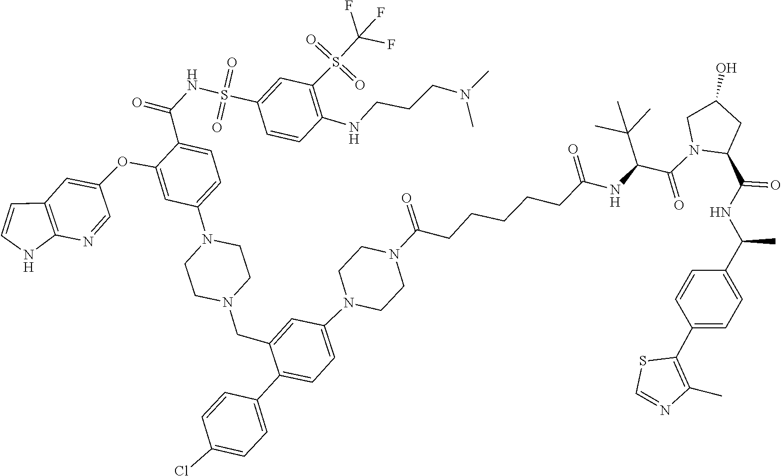 [SMILES: Cc1ncsc1-c1ccc([C@H](C)NC(=O)[C@@H]2C[C@@H](O)CN2C(=O)[C@@H](NC(=O)CCCCCC(=O)N2CCN(c3ccc(-c4ccc(Cl)cc4)c(CN4CCN(c5ccc(C(=O)NS(=O)(=O)c6ccc(NCCCN(C)C)c(S(=O)(=O)C(F)(F)F)c6)c(Oc6cnc7[nH]ccc7c6)c5)CC4)c3)CC2)C(C)(C)C)cc1] |
| I-100 | 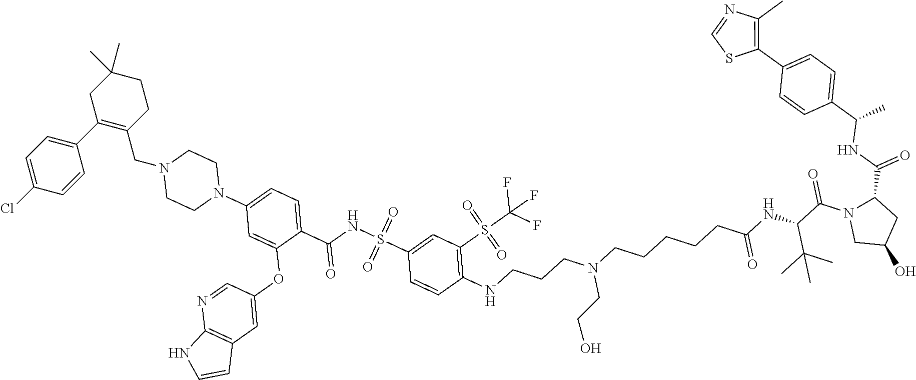 [SMILES: Cc1ncsc1-c1ccc([C@H](C)NC(=O)[C@@H]2C[C@@H](O)CN2C(=O)[C@@H](NC(=O)CCCCCN(CCO)CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-101 | 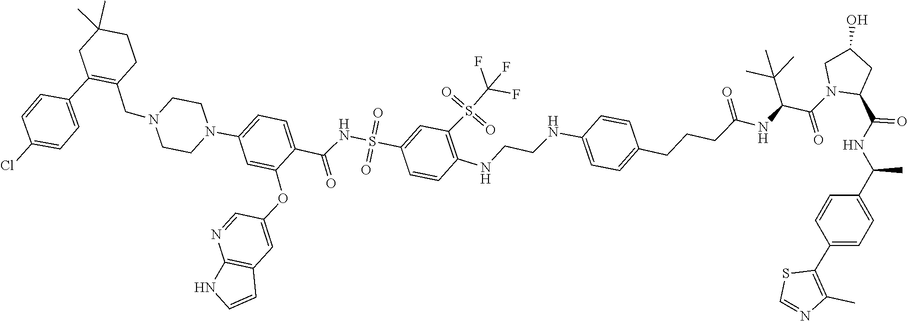 [SMILES: Cc1ncsc1-c1ccc([C@H](C)NC(=O)[C@@H]2C[C@@H](O)CN2C(=O)[C@@H](NC(=O)CCCc2ccc(NCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)cc2)C(C)(C)C)cc1] |
| I-102 | 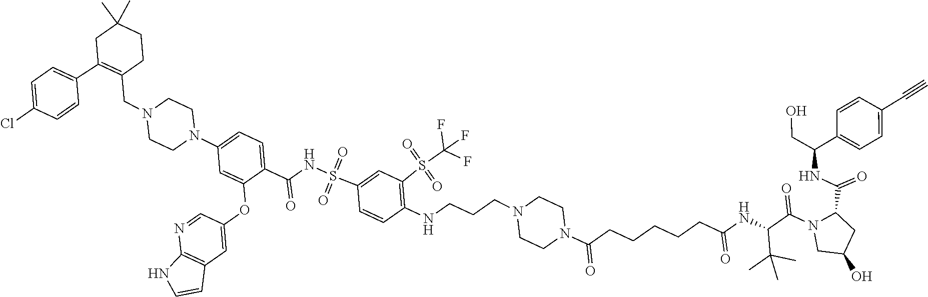 [SMILES: C#Cc1ccc([C@H](CO)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-103 | 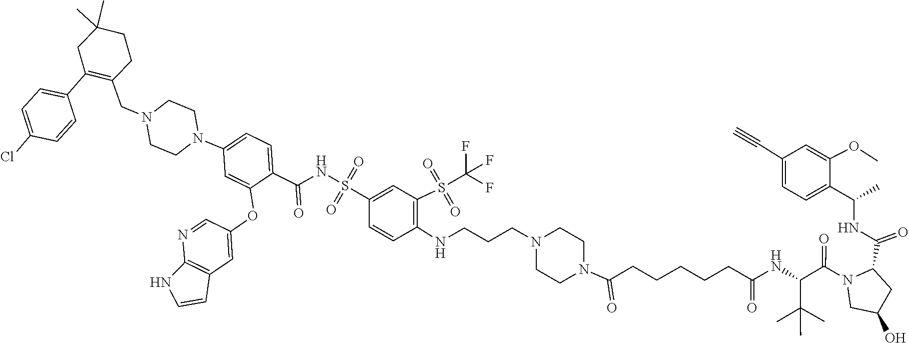 [SMILES: C#Cc1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)c(OC)c1] |
| I-104 | 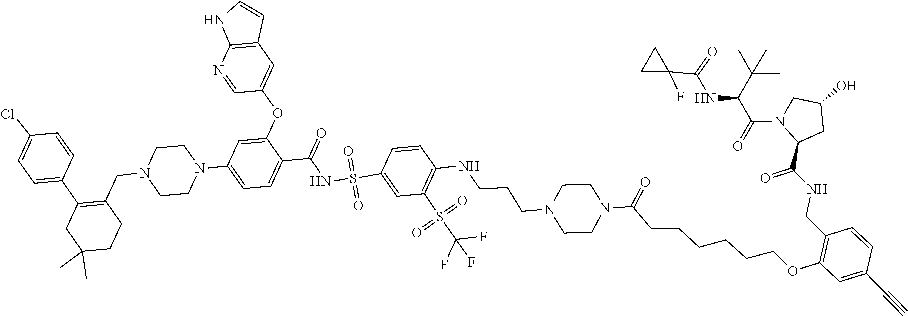 [SMILES: C#Cc1ccc(CNC(=O)[C@@H]2C[C@@H](O)CN2C(=O)[C@@H](NC(=O)C2(F)CC2)C(C)(C)C)c(OCCCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)c1] |
| I-105 | 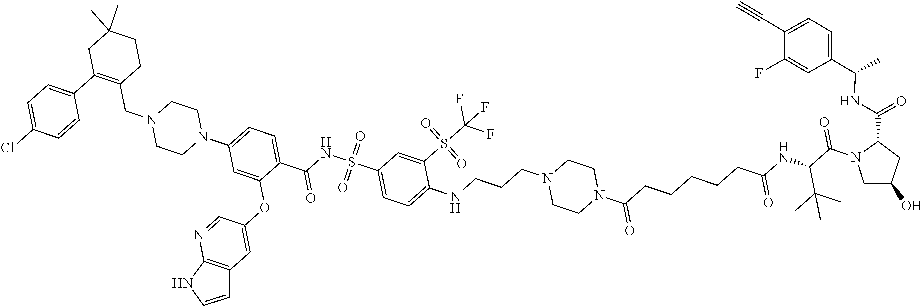 [SMILES: C#Cc1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1F] |
| I-106 | 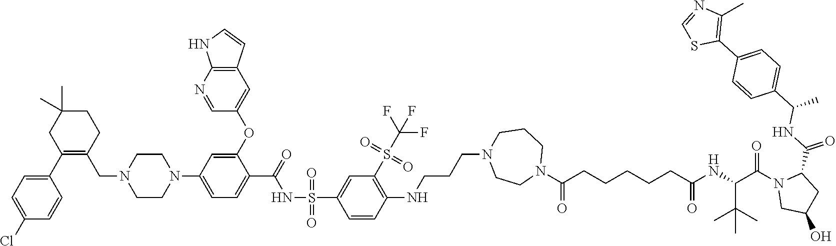 [SMILES: Cc1ncsc1-c1ccc([C@H](C)NC(=O)[C@@H]2C[C@@H](O)CN2C(=O)[C@@H](NC(=O)CCCCCC(=O)N2CCCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-107 | 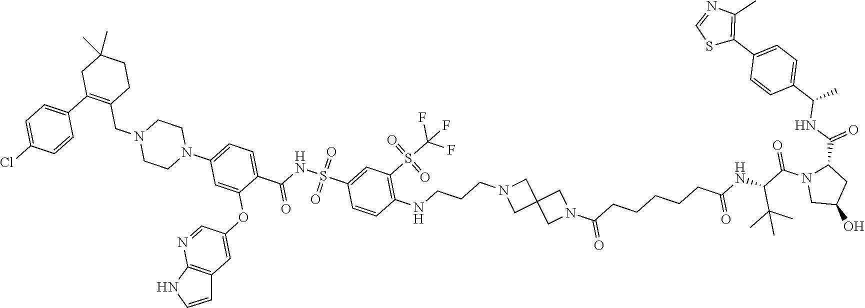 [SMILES: Cc1ncsc1-c1ccc([C@H](C)NC(=O)[C@@H]2C[C@@H](O)CN2C(=O)[C@@H](NC(=O)CCCCCC(=O)N2CC3(CN(CCCNc4ccc(S(=O)(=O)NC(=O)c5ccc(N6CCN(CC7=C(c8ccc(Cl)cc8)CC(C)(C)CC7)CC6)cc5Oc5cnc6[nH]ccc6c5)cc4S(=O)(=O)C(F)(F)F)C3)C2)C(C)(C)C)cc1] |
| I-108 | 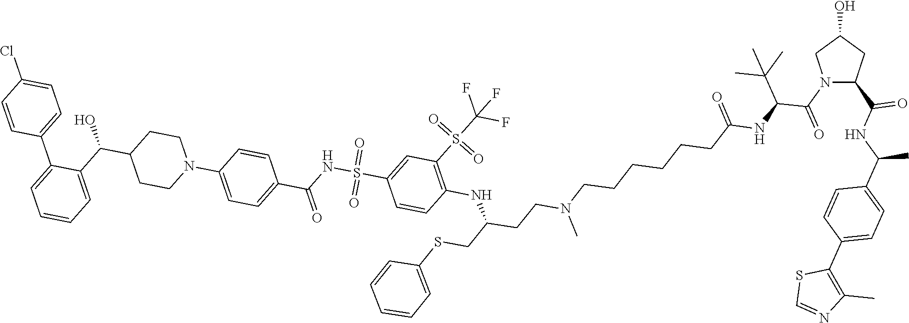 [SMILES: Cc1ncsc1-c1ccc([C@H](C)NC(=O)[C@@H]2C[C@@H](O)CN2C(=O)[C@@H](NC(=O)CCCCCCN(C)CC[C@H](CSc2ccccc2)Nc2ccc(S(=O)(=O)NC(=O)c3ccc(N4CCC([C@@H](O)c5ccccc5-c5ccc(Cl)cc5)CC4)cc3)cc2S(=O)(=O)C(F)(F)F)C(C)(C)C)cc1] |
| I-109 | 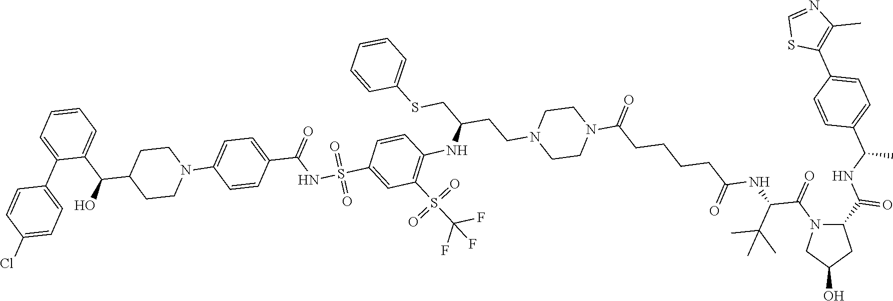 [SMILES: Cc1ncsc1-c1ccc([C@H](C)NC(=O)[C@@H]2C[C@@H](O)CN2C(=O)[C@@H](NC(=O)CCCCC(=O)N2CCN(CC[C@H](CSc3ccccc3)Nc3ccc(S(=O)(=O)NC(=O)c4ccc(N5CCC([C@@H](O)c6ccccc6-c6ccc(Cl)cc6)CC5)cc4)cc3S(=O)(=O)C(F)(F)F)CC2)C(C)(C)C)cc1] |
| I-110 | 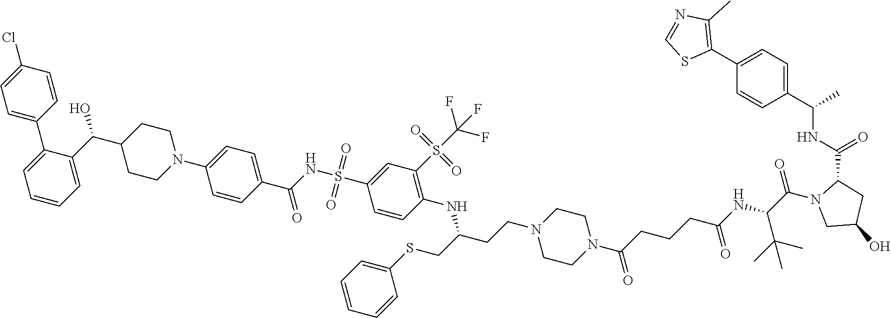 [SMILES: Cc1ncsc1-c1ccc([C@H](C)NC(=O)[C@@H]2C[C@@H](O)CN2C(=O)[C@@H](NC(=O)CCCC(=O)N2CCN(CC[C@H](CSc3ccccc3)Nc3ccc(S(=O)(=O)NC(=O)c4ccc(N5CCC([C@@H](O)c6ccccc6-c6ccc(Cl)cc6)CC5)cc4)cc3S(=O)(=O)C(F)(F)F)CC2)C(C)(C)C)cc1] |
| I-111 | 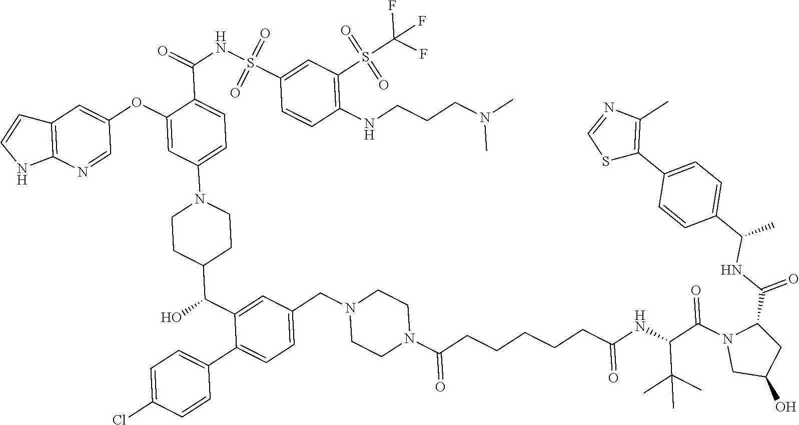 [SMILES: Cc1ncsc1-c1ccc([C@H](C)NC(=O)[C@@H]2C[C@@H](O)CN2C(=O)[C@@H](NC(=O)CCCCCC(=O)N2CCN(Cc3ccc(-c4ccc(Cl)cc4)c([C@H](O)C4CCN(c5ccc(C(=O)NS(=O)(=O)c6ccc(NCCCN(C)C)c(S(=O)(=O)C(F)(F)F)c6)c(Oc6cnc7[nH]ccc7c6)c5)CC4)c3)CC2)C(C)(C)C)cc1] |
| I-112 | 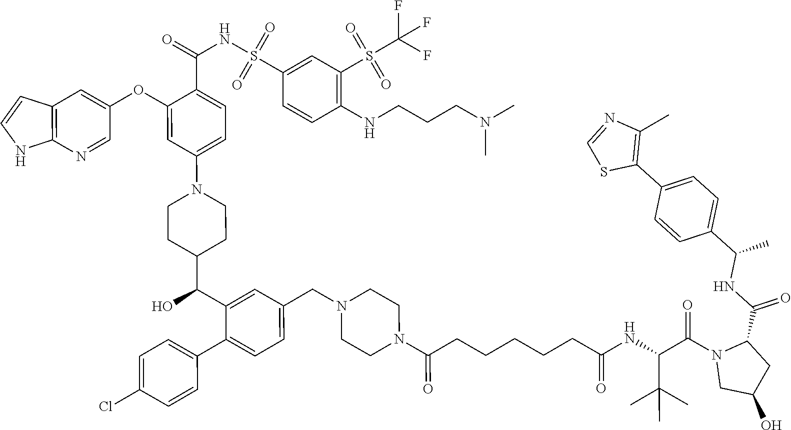 [SMILES: Cc1ncsc1-c1ccc([C@H](C)NC(=O)[C@@H]2C[C@@H](O)CN2C(=O)[C@@H](NC(=O)CCCCCC(=O)N2CCN(Cc3ccc(-c4ccc(Cl)cc4)c([C@@H](O)C4CCN(c5ccc(C(=O)NS(=O)(=O)c6ccc(NCCCN(C)C)c(S(=O)(=O)C(F)(F)F)c6)c(Oc6cnc7[nH]ccc7c6)c5)CC4)c3)CC2)C(C)(C)C)cc1] |
| I-113 | 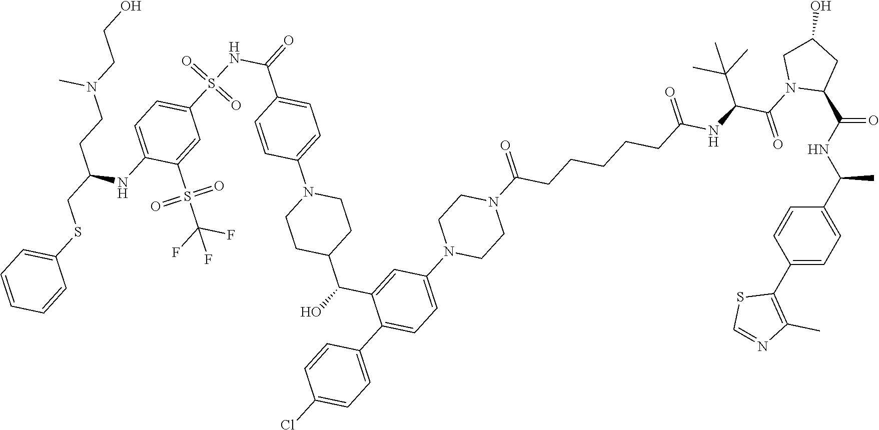 [SMILES: Cc1ncsc1-c1ccc([C@H](C)NC(=O)[C@@H]2C[C@@H](O)CN2C(=O)[C@@H](NC(=O)CCCCCC(=O)N2CCN(c3ccc(-c4ccc(Cl)cc4)c([C@H](O)C4CCN(c5ccc(C(=O)NS(=O)(=O)c6ccc(N[C@H](CCN(C)CCO)CSc7ccccc7)c(S(=O)(=O)C(F)(F)F)c6)cc5)CC4)c3)CC2)C(C)(C)C)cc1] |
| I-114 | 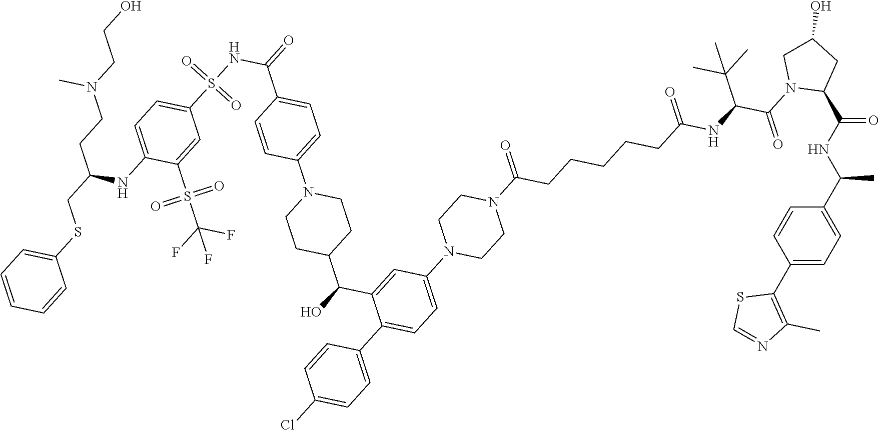 [SMILES: Cc1ncsc1-c1ccc([C@H](C)NC(=O)[C@@H]2C[C@@H](O)CN2C(=O)[C@@H](NC(=O)CCCCCC(=O)N2CCN(c3ccc(-c4ccc(Cl)cc4)c([C@@H](O)C4CCN(c5ccc(C(=O)NS(=O)(=O)c6ccc(N[C@H](CCN(C)CCO)CSc7ccccc7)c(S(=O)(=O)C(F)(F)F)c6)cc5)CC4)c3)CC2)C(C)(C)C)cc1] |
| I-115 | 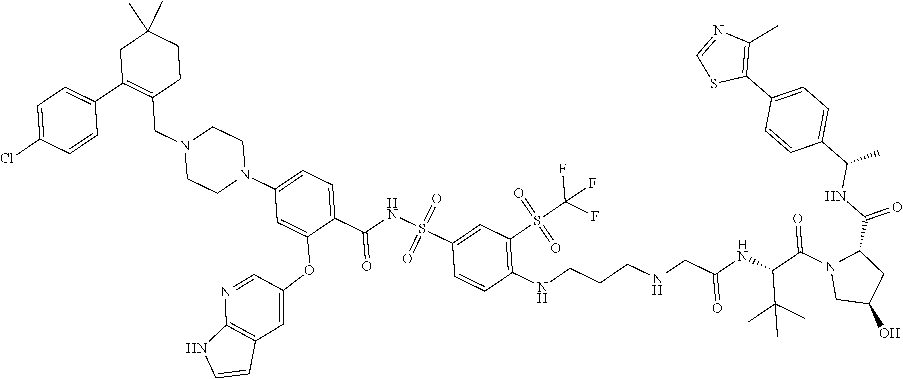 [SMILES: Cc1ncsc1-c1ccc([C@H](C)NC(=O)[C@@H]2C[C@@H](O)CN2C(=O)[C@@H](NC(=O)CNCCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-116 | 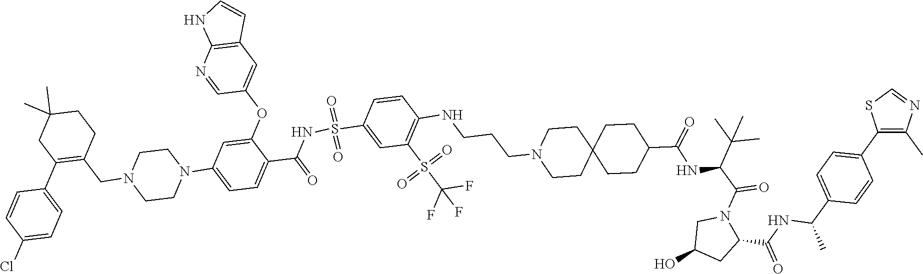 [SMILES: Cc1ncsc1-c1ccc([C@H](C)NC(=O)[C@@H]2C[C@@H](O)CN2C(=O)[C@@H](NC(=O)C2CCC3(CC2)CCN(CCCNc2ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc2S(=O)(=O)C(F)(F)F)CC3)C(C)(C)C)cc1] |
| I-117 | 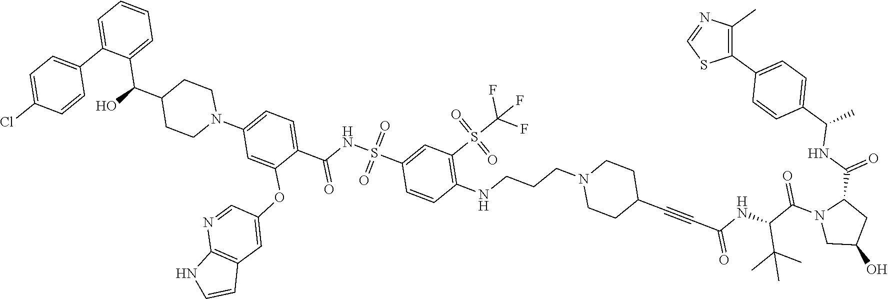 [SMILES: Cc1ncsc1-c1ccc([C@H](C)NC(=O)[C@@H]2C[C@@H](O)CN2C(=O)[C@@H](NC(=O)C#CC2CCN(CCCNc3ccc(S(=O)(=O)NC(=O)c4ccc(N5CCC([C@@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-118 | 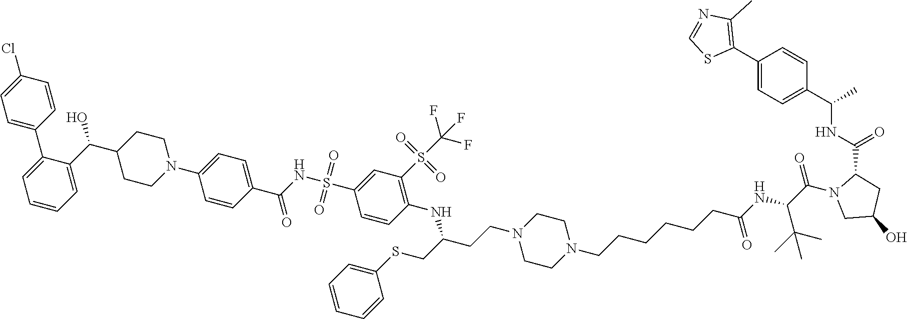 [SMILES: Cc1ncsc1-c1ccc([C@H](C)NC(=O)[C@@H]2C[C@@H](O)CN2C(=O)[C@@H](NC(=O)CCCCCCN2CCN(CC[C@H](CSc3ccccc3)Nc3ccc(S(=O)(=O)NC(=O)c4ccc(N5CCC([C@@H](O)c6ccccc6-c6ccc(Cl)cc6)CC5)cc4)cc3S(=O)(=O)C(F)(F)F)CC2)C(C)(C)C)cc1] |
| I-119 | 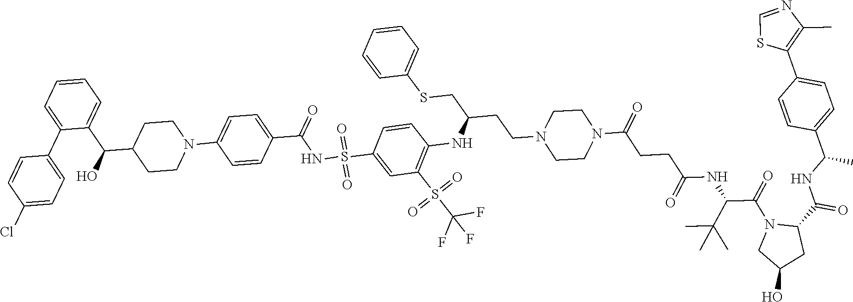 [SMILES: Cc1ncsc1-c1ccc([C@H](C)NC(=O)[C@@H]2C[C@@H](O)CN2C(=O)[C@@H](NC(=O)CCC(=O)N2CCN(CC[C@H](CSc3ccccc3)Nc3ccc(S(=O)(=O)NC(=O)c4ccc(N5CCC([C@@H](O)c6ccccc6-c6ccc(Cl)cc6)CC5)cc4)cc3S(=O)(=O)C(F)(F)F)CC2)C(C)(C)C)cc1] |
| I-120 | 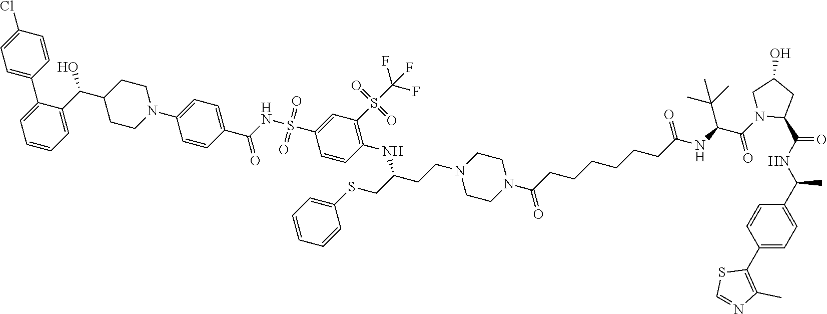 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC[C@H](CSc3ccccc3)Nc3ccc(S(=O)(=O)NC(=O)c4ccc(N5CCC([C@@H](O)c6ccccc6-c6ccc(Cl)cc6)CC5)cc4)cc3S(=O)(=O)C(F)(F)F)CC2)C(C)(C)C)cc1] |
| I-121 | 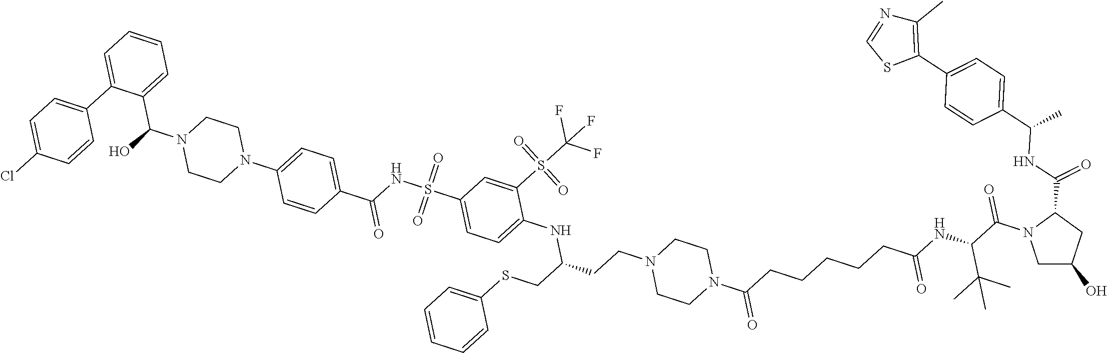 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN([C@@H](O)c6ccccc6-c6ccc(Cl)cc6)CC5)cc4)cc3S(=O)(=O)C(F)(F)F)CC2)C(C)(C)C)cc1] |
| I-122 | 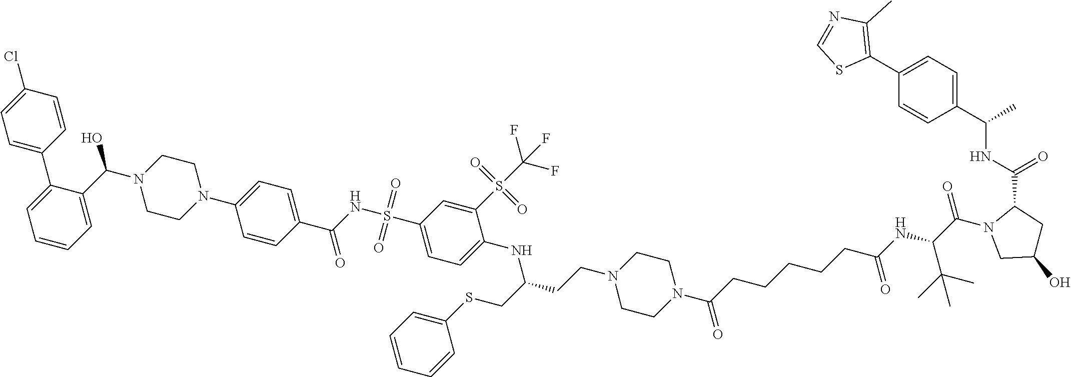 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN([C@H](O)c6ccccc6-c6ccc(Cl)cc6)CC5)cc4)cc3S(=O)(=O)C(F)(F)F)CC2)C(C)(C)C)cc1] |
| I-123 | 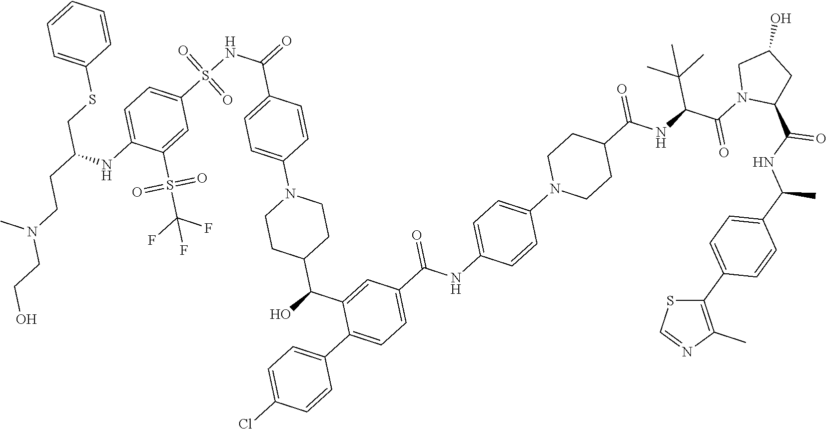 [SMILES: Cc1ncsc1-c1ccc([C@H](C)NC(=O)[C@@H]2C[C@@H](O)CN2C(=O)[C@@H](NC(=O)C2CCN(c3ccc(NC(=O)c4ccc(-c5ccc(Cl)cc5)c([C@@H](O)C5CCN(c6ccc(C(=O)NS(=O)(=O)c7ccc(N[C@H](CCN(C)CCO)CSc8ccccc8)c(S(=O)(=O)C(F)(F)F)c7)cc6)CC5)c4)cc3)CC2)C(C)(C)C)cc1] |
| I-124 | 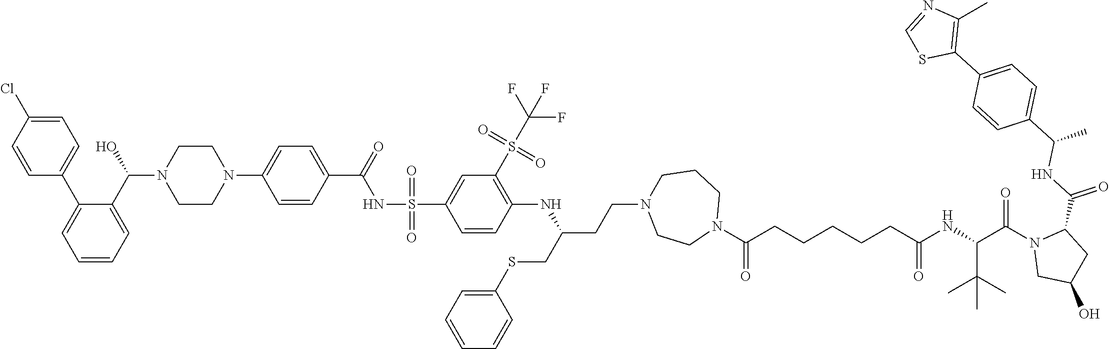 [SMILES: Cc1ncsc1-c1ccc([C@H](C)NC(=O)[C@@H]2C[C@@H](O)CN2C(=O)[C@@H](NC(=O)CCCCCC(=O)N2CCCN(CC[C@H](CSc3ccccc3)Nc3ccc(S(=O)(=O)NC(=O)c4ccc(N5CCN([C@@H](O)c6ccccc6-c6ccc(Cl)cc6)CC5)cc4)cc3S(=O)(=O)C(F)(F)F)CC2)C(C)(C)C)cc1] |
| I-125 | 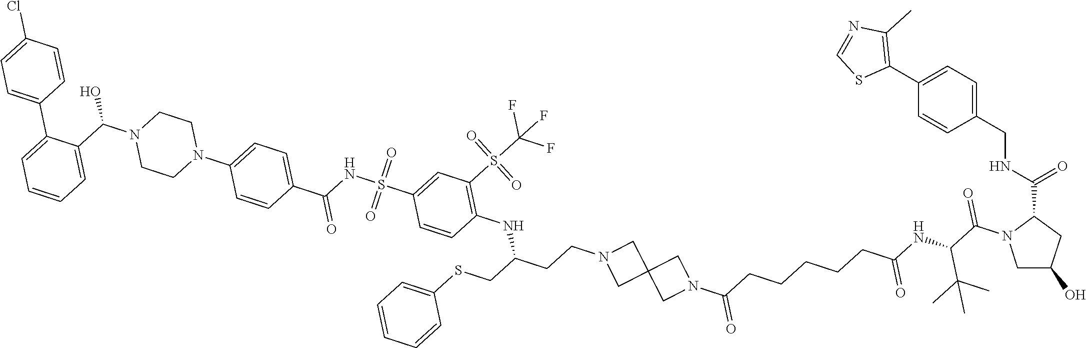 [SMILES: Cc1ncsc1-c1ccc(CNC(=O)[C@@H]2C[C@@H](O)CN2C(=O)[C@@H](NC(=O)CCCCCC(=O)N2CC3(CN(CC[C@H](CSc4ccccc4)Nc4ccc(S(=O)(=O)NC(=O)c5ccc(N6CCN([C@@H](O)c7ccccc7-c7ccc(Cl)cc7)CC6)cc5)cc4S(=O)(=O)C(F)(F)F)C3)C2)C(C)(C)C)cc1] |
| I-126 | 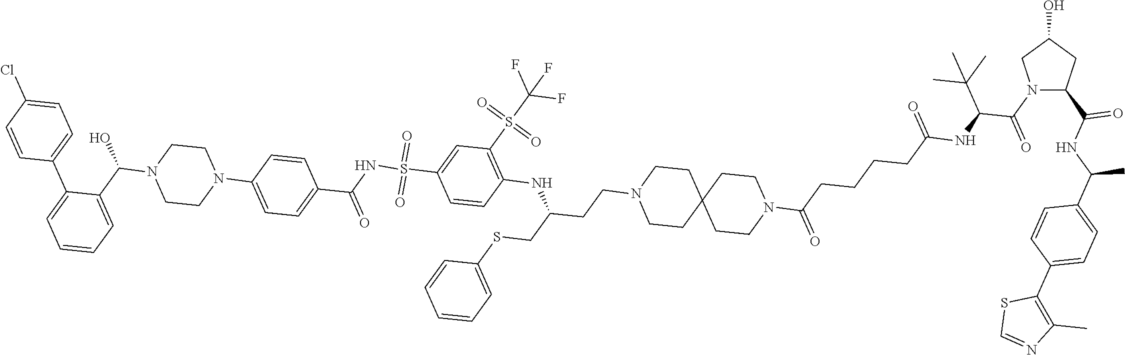 [SMILES: Cc1ncsc1-c1ccc([C@H](C)NC(=O)[C@@H]2C[C@@H](O)CN2C(=O)[C@@H](NC(=O)CCCCC(=O)N2CCC3(CCN(CC[C@H](CSc4ccccc4)Nc4ccc(S(=O)(=O)NC(=O)c5ccc(N6CCN([C@@H](O)c7ccccc7-c7ccc(Cl)cc7)CC6)cc5)cc4S(=O)(=O)C(F)(F)F)CC3)CC2)C(C)(C)C)cc1] |
| I-127 | 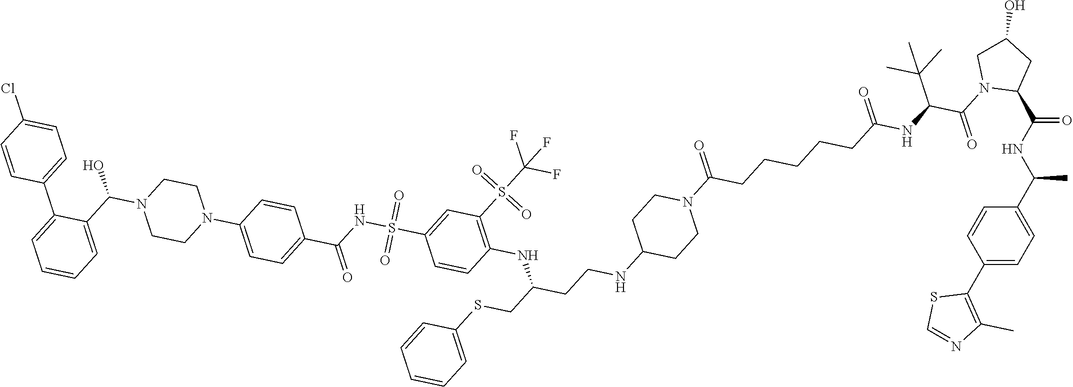 [SMILES: Cc1ncsc1-c1ccc([C@H](C)NC(=O)[C@@H]2C[C@@H](O)CN2C(=O)[C@@H](NC(=O)CCCCCC(=O)N2CCC(NCC[C@H](CSc3ccccc3)Nc3ccc(S(=O)(=O)NC(=O)c4ccc(N5CCN([C@@H](O)c6ccccc6-c6ccc(Cl)cc6)CC5)cc4)cc3S(=O)(=O)C(F)(F)F)CC2)C(C)(C)C)cc1] |
| I-128 | 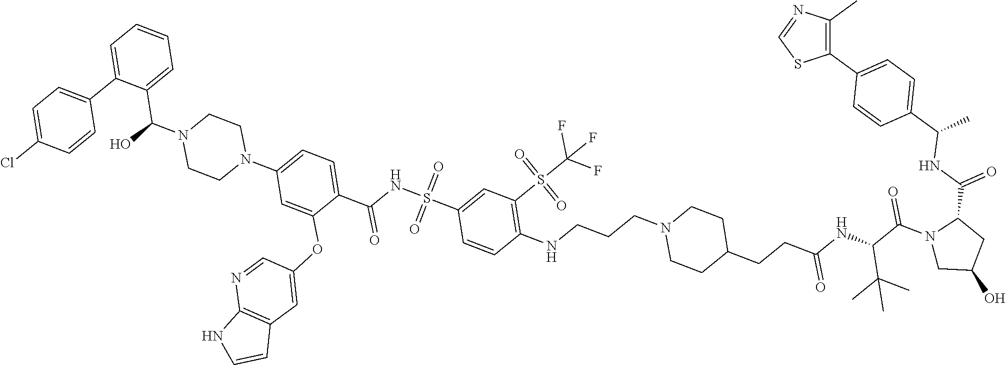 [SMILES: Cc1ncsc1-c1ccc([C@H](C)NC(=O)[C@@H]2C[C@@H](O)CN2C(=O)[C@@H](NC(=O)CCC2CCN(CCCNc3ccc(S(=O)(=O)NC(=O)c4ccc(N5CCN([C@@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-129 | 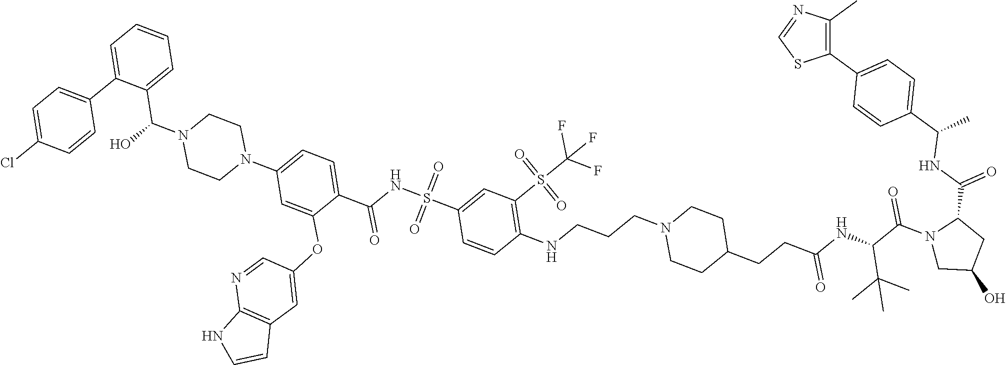 [SMILES: Cc1ncsc1-c1ccc([C@H](C)NC(=O)[C@@H]2C[C@@H](O)CN2C(=O)[C@@H](NC(=O)CCC2CCN(CCCNc3ccc(S(=O)(=O)NC(=O)c4ccc(N5CCN([C@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-130 | 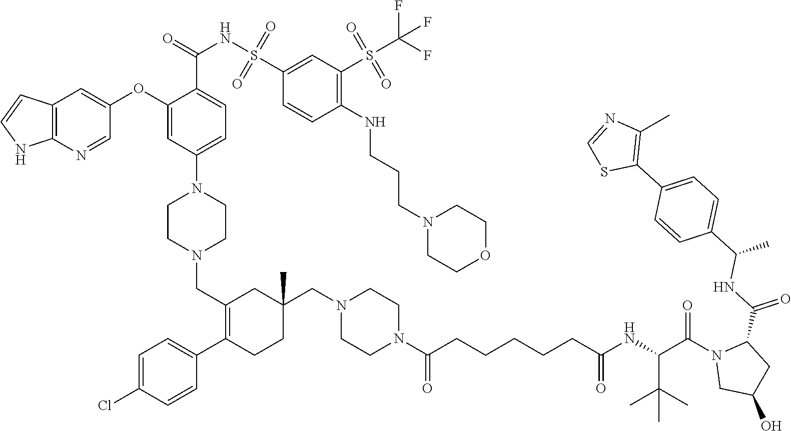 [SMILES: Cc1ncsc1-c1ccc([C@H](C)NC(=O)[C@@H]2C[C@@H](O)CN2C(=O)[C@@H](NC(=O)CCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-131 | 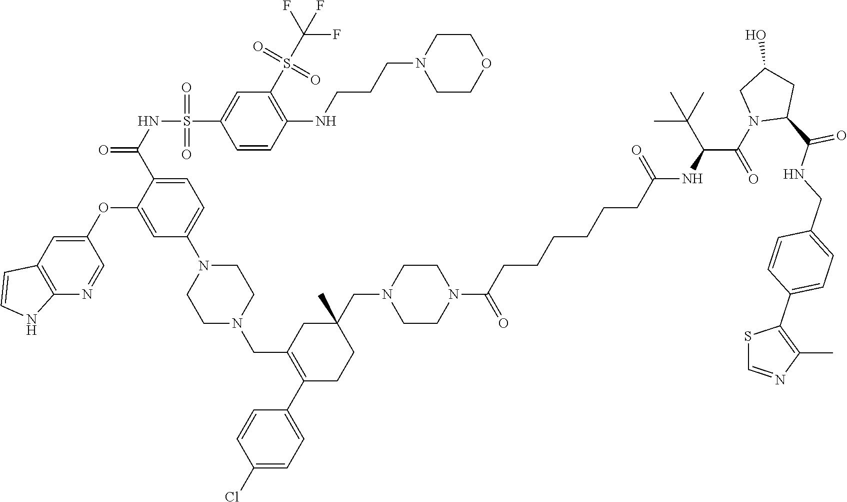 [SMILES: Cc1ncsc1-c1ccc(CNC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-132 | 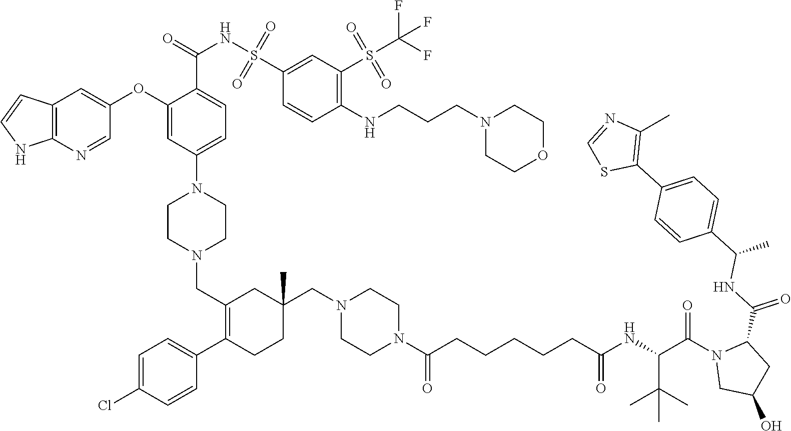 [SMILES: Cc1ncsc1-c1ccc([C@H](C)NC(=O)[C@@H]2C[C@@H](O)CN2C(=O)[C@@H](NC(=O)CCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-133 | 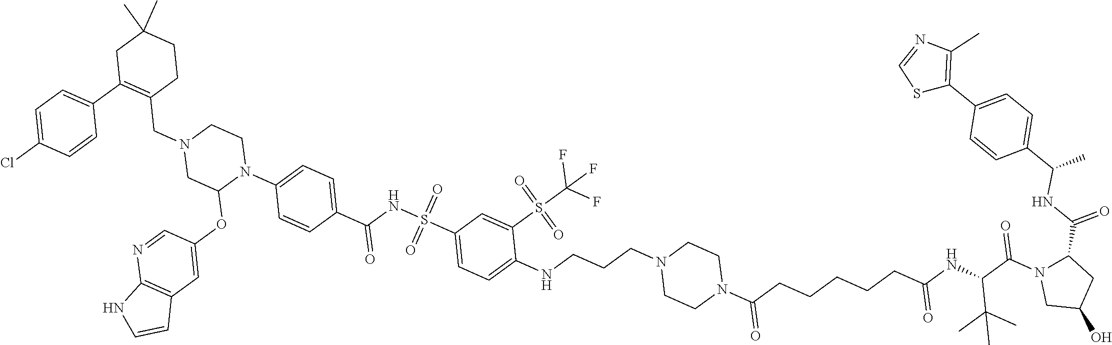 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CC(C)(C)CC6)CC5Oc5cnc6[nH]ccc6c5)cc4)cc3S(=O)(=O)C(F)(F)F)CC2)C(C)(C)C)cc1] |
| I-134 | 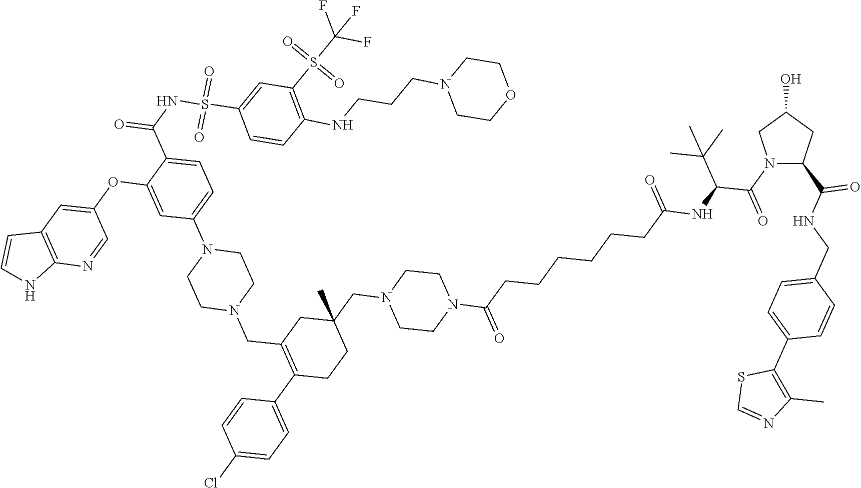 [SMILES: Cc1ncsc1-c1ccc(CNC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-135 | 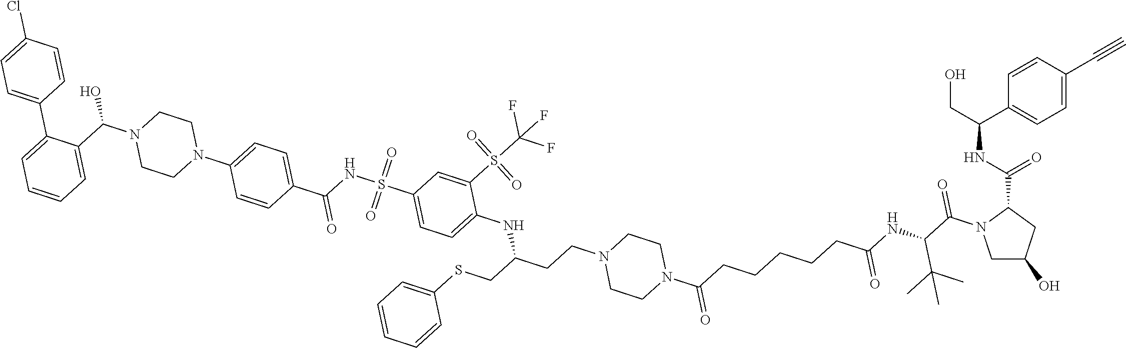 [SMILES: C#Cc1ccc([C@H](CO)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN([C@@H](O)c6ccccc6-c6ccc(Cl)cc6)CC5)cc4)cc3S(=O)(=O)C(F)(F)F)CC2)C(C)(C)C)cc1] |
| I-136 | 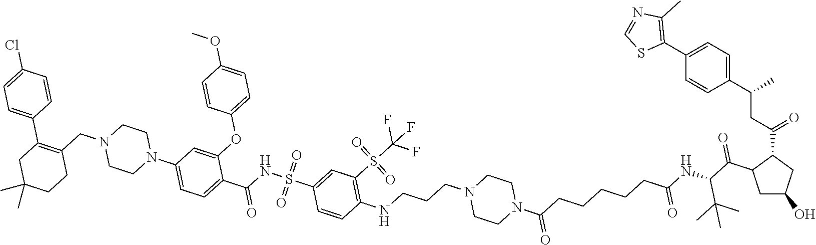 [SMILES: COc1ccc(Oc2cc(N3CCN(CC4=C(c5ccc(Cl)cc5)CC(C)(C)CC4)CC3)ccc2C(=O)NS(=O)(=O)c2ccc(NCCCN3CCN(C(=O)CCCCCC(=O)N[C@H](C(=O)C4C[C@H](O)C[C@H]4C(=O)C[C@@H](C)c4ccc(-c5scnc5C)cc4)C(C)(C)C)CC3)c(S(=O)(=O)C(F)(F)F)c2)cc1] |
| I-137 | 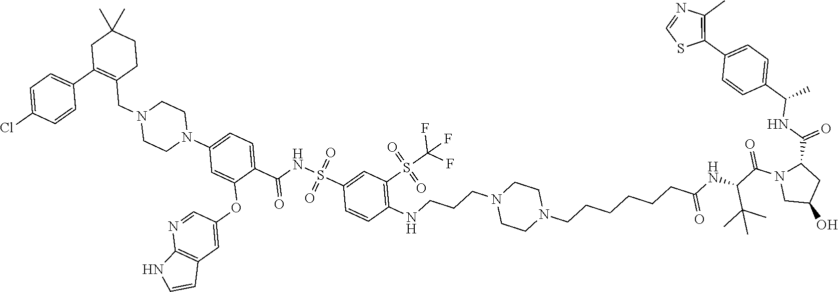 [SMILES: Cc1ncsc1-c1ccc([C@H](C)NC(=O)[C@@H]2C[C@@H](O)CN2C(=O)[C@@H](NC(=O)CCCCCCN2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-138 | 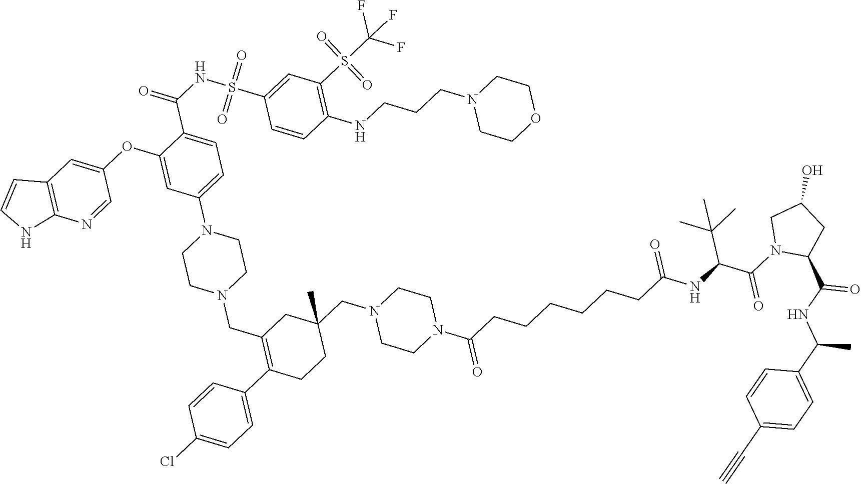 [SMILES: C#Cc1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-139 | 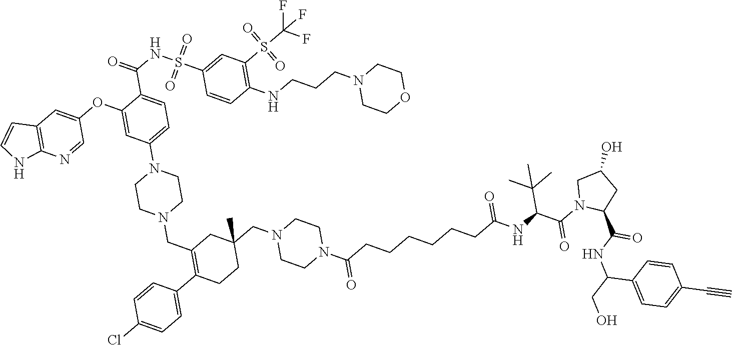 [SMILES: C#Cc1ccc(C(CO)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-140 | 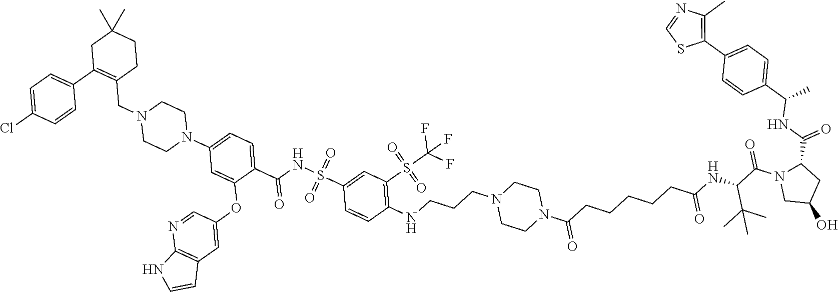 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-141 | 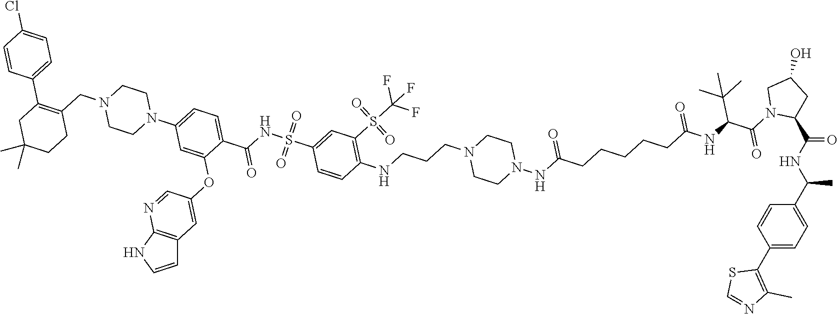 [SMILES: Cc1ncsc1-c1ccc([C@H](C)NC(=O)[C@@H]2C[C@@H](O)CN2C(=O)[C@@H](NC(=O)CCCCCC(=O)NN2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-142 | 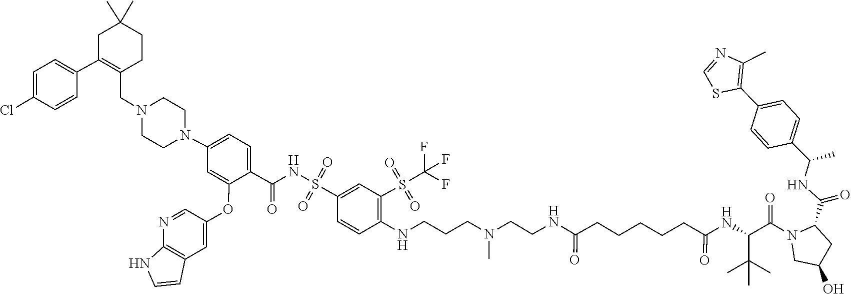 [SMILES: Cc1ncsc1-c1ccc([C@H](C)NC(=O)[C@@H]2C[C@@H](O)CN2C(=O)[C@@H](NC(=O)CCCCCC(=O)NCCN(C)CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-143 | 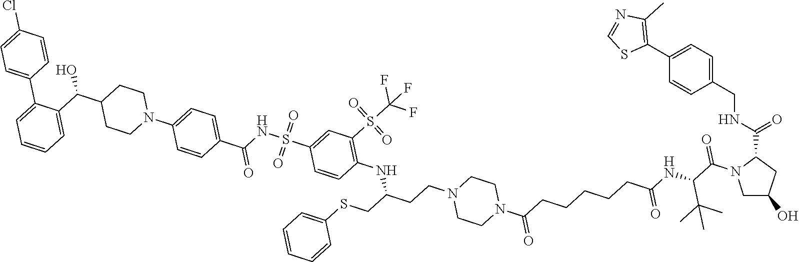 [SMILES: Cc1ncsc1-c1ccc(CNC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCC([C@@H](O)c6ccccc6-c6ccc(Cl)cc6)CC5)cc4)cc3S(=O)(=O)C(F)(F)F)CC2)C(C)(C)C)cc1] |
| I-144 | 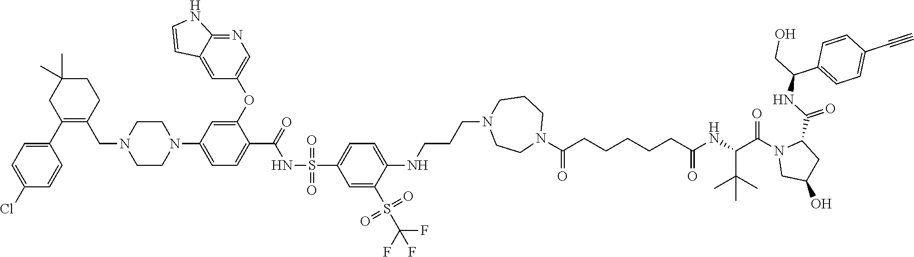 [SMILES: C#Cc1ccc([C@H](CO)NC(=O)[C@@H]2C[C@@H](O)CN2C(=O)[C@@H](NC(=O)CCCCCC(=O)N2CCCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-145 | 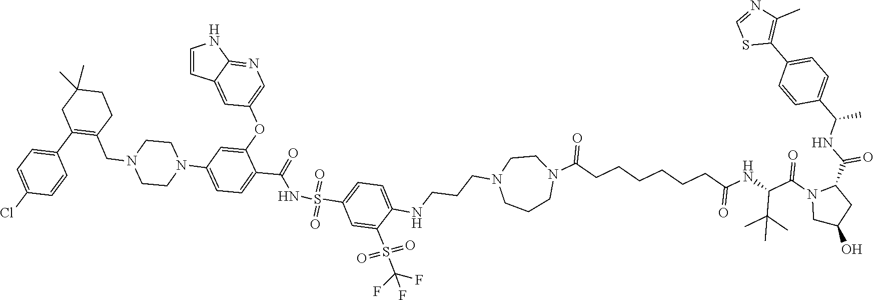 [SMILES: Cc1ncsc1-c1ccc([C@H](C)NC(=O)[C@@H]2C[C@@H](O)CN2C(=O)[C@@H](NC(=O)CCCCCCC(=O)N2CCCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-146 | 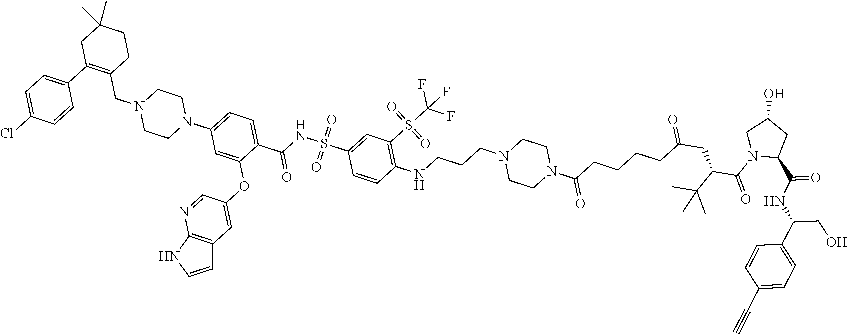 [SMILES: C#Cc1ccc([C@@H](CO)NC(=O)[C@@H]2C[C@@H](O)CN2C(=O)[C@@H](CC(=O)CCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-147 | 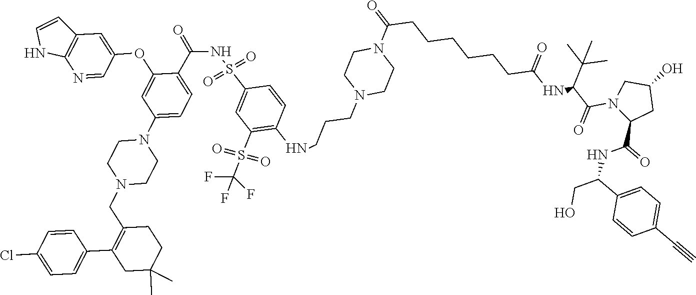 [SMILES: C#Cc1ccc([C@H](CO)NC(=O)[C@@H]2C[C@@H](O)CN2C(=O)[C@@H](NC(=O)CCCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-148 | 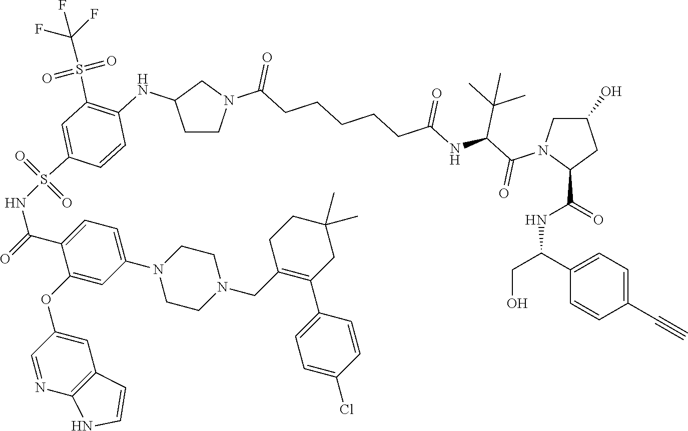 [SMILES: C#Cc1ccc([C@H](CO)NC(=O)[C@@H]2C[C@@H](O)CN2C(=O)[C@@H](NC(=O)CCCCCC(=O)N2CCC(Nc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)C2)C(C)(C)C)cc1] |
| I-149 | 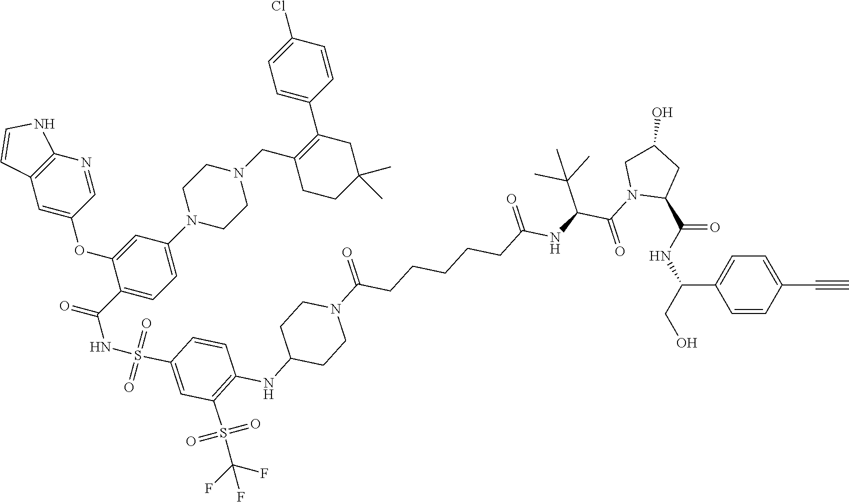 [SMILES: C#Cc1ccc([C@H](CO)NC(=O)[C@@H]2C[C@@H](O)CN2C(=O)[C@@H](NC(=O)CCCCCC(=O)N2CCC(Nc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-150 | 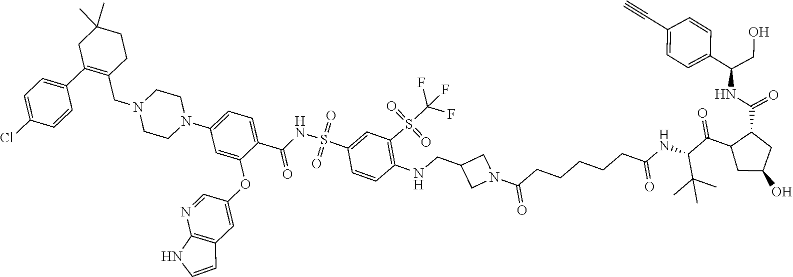 [SMILES: C#Cc1ccc([C@@H](CO)NC(=O)[C@@H]2C[C@@H](O)CC2C(=O)[C@@H](NC(=O)CCCCCC(=O)N2CC(CNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)C2)C(C)(C)C)cc1] |
| I-151 | 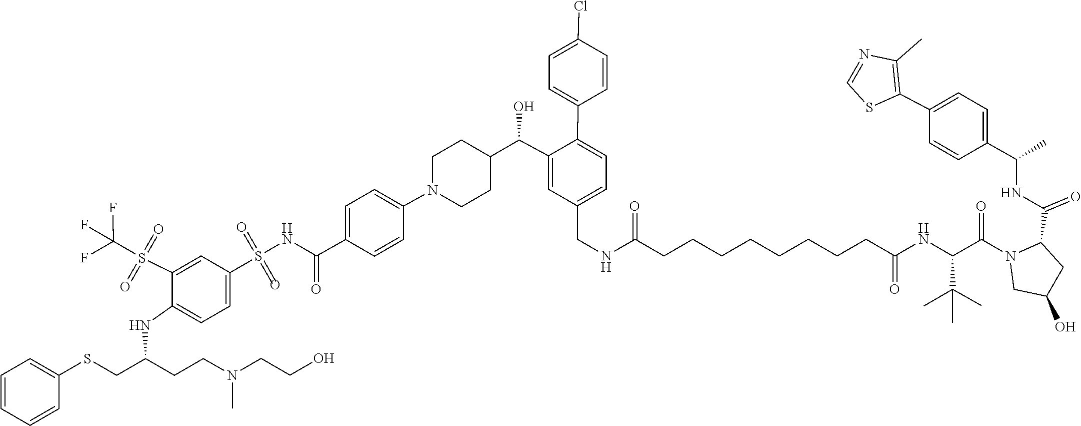 |
| I-152 | 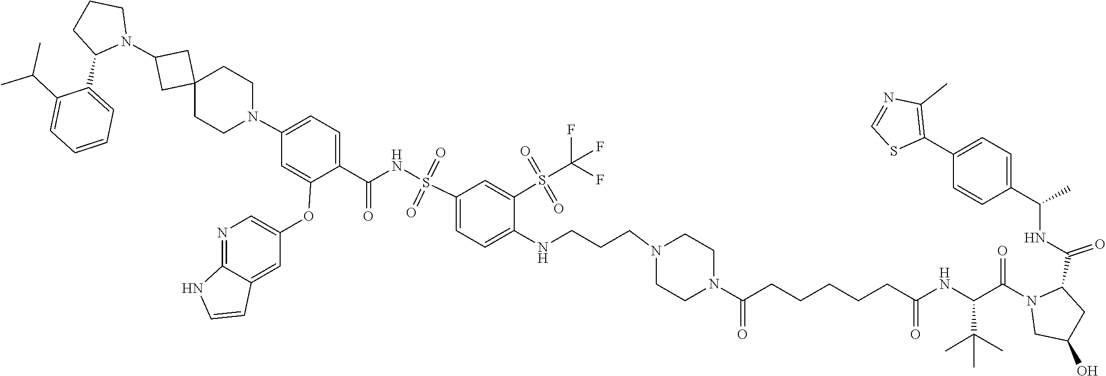 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCC6(CC5)CC(N5CCC[C@H]5c5ccccc5C(C)C)C6)cc4Oc4cnc5[nH]ccc5c4)cc3S(=O)(=O)C(F)(F)F)CC2)C(C)(C)C)cc1] |
| I-153 | 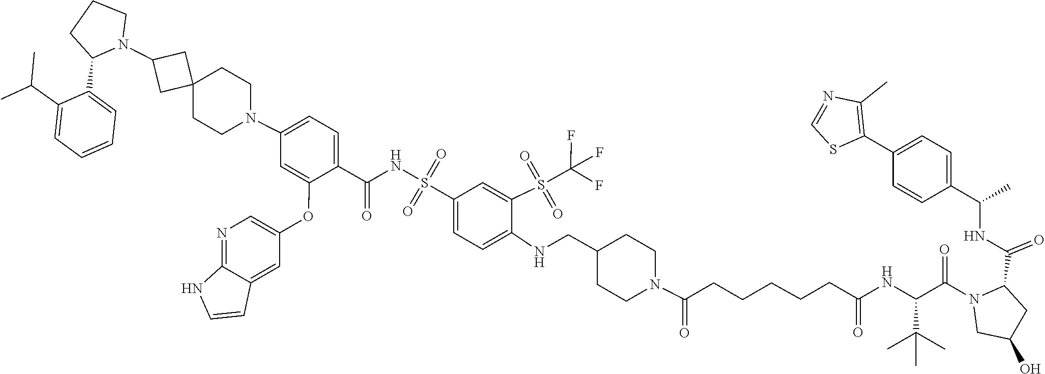 [SMILES: Cc1ncsc1-c1ccc([C@H](C)NC(=O)[C@@H]2C[C@@H](O)CN2C(=O)[C@@H](NC(=O)CCCCCC(=O)N2CCC(CNc3ccc(S(=O)(=O)NC(=O)c4ccc(N5CCC6(CC5)CC(N5CCC[C@H]5c5ccccc5C(C)C)C6)cc4Oc4cnc5[nH]ccc5c4)cc3S(=O)(=O)C(F)(F)F)CC2)C(C)(C)C)cc1] |
| I-154 | 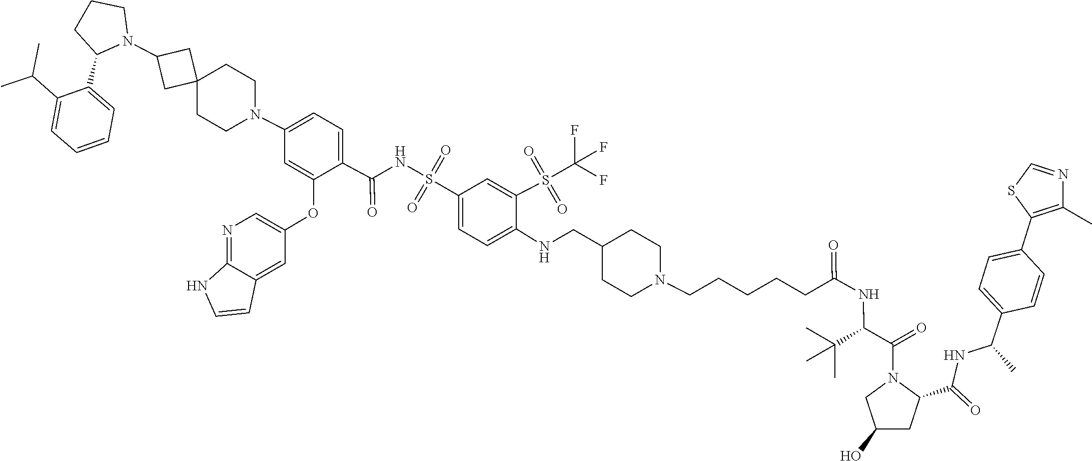 [SMILES: Cc1ncsc1-c1ccc([C@H](C)NC(=O)[C@@H]2C[C@@H](O)CN2C(=O)[C@@H](NC(=O)CCCCCN2CCC(CNc3ccc(S(=O)(=O)NC(=O)c4ccc(N5CCC6(CC5)CC(N5CCC[C@H]5c5ccccc5C(C)C)C6)cc4Oc4cnc5[nH]ccc5c4)cc3S(=O)(=O)C(F)(F)F)CC2)C(C)(C)C)cc1] |
| I-155 | 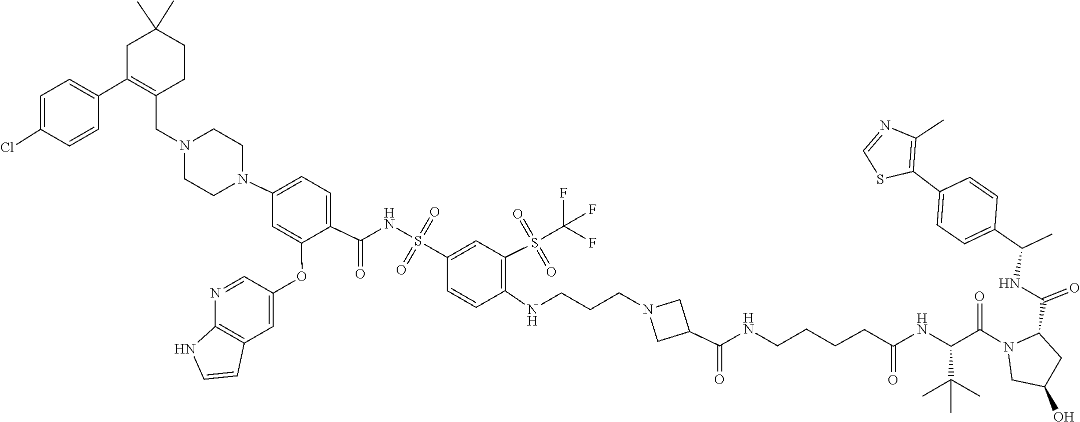 [SMILES: Cc1ncsc1-c1ccc([C@H](C)NC(=O)[C@@H]2C[C@@H](O)CN2C(=O)[C@@H](NC(=O)CCCCNC(=O)C2CN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)C2)C(C)(C)C)cc1] |
| I-156 | 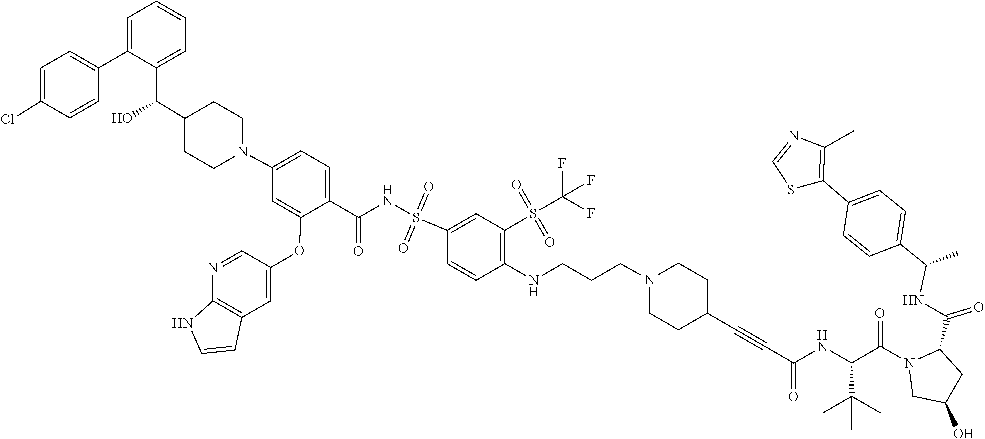 [SMILES: Cc1ncsc1-c1ccc([C@H](C)NC(=O)[C@@H]2C[C@@H](O)CN2C(=O)[C@@H](NC(=O)C#CC2CCN(CCCNc3ccc(S(=O)(=O)NC(=O)c4ccc(N5CCC([C@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-157 | 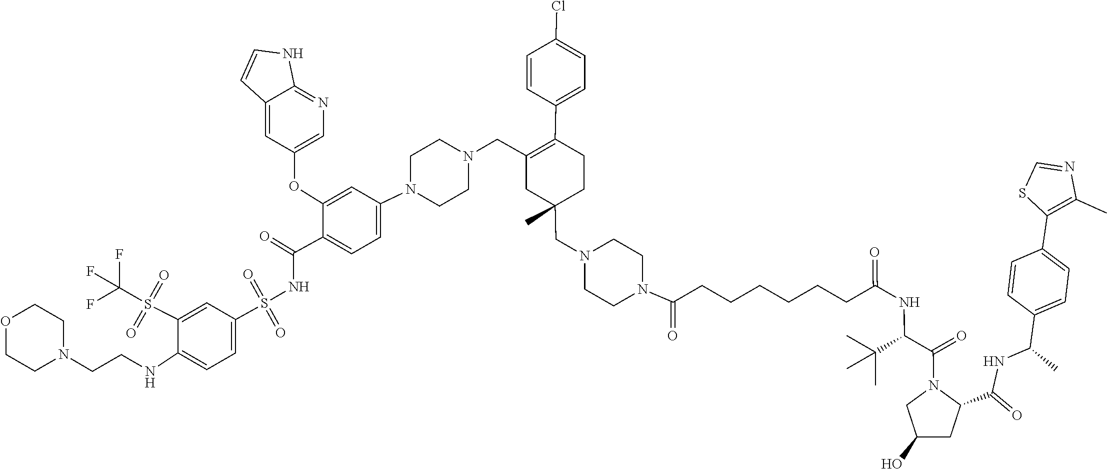 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@@]3(C)CCC(c4ccc(Cl)cc4)=C(CN4CCN(c5ccc(C(=O)NS(=O)(=O)c6ccc(NCCN7CCOCC7)c(S(=O)(=O)C(F)(F)F)c6)c(Oc6cnc7[nH]ccc7c6)c5)CC4)C3)CC2)C(C)(C)C)cc1] |
| I-158 | 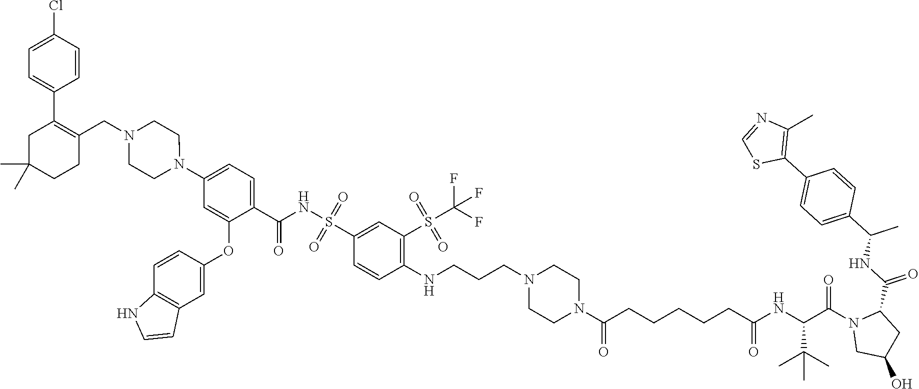 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4ccc5[nH]ccc5c4)cc3S(=O)(=O)C(F)(F)F)CC2)C(C)(C)C)cc1] |
| I-159 | 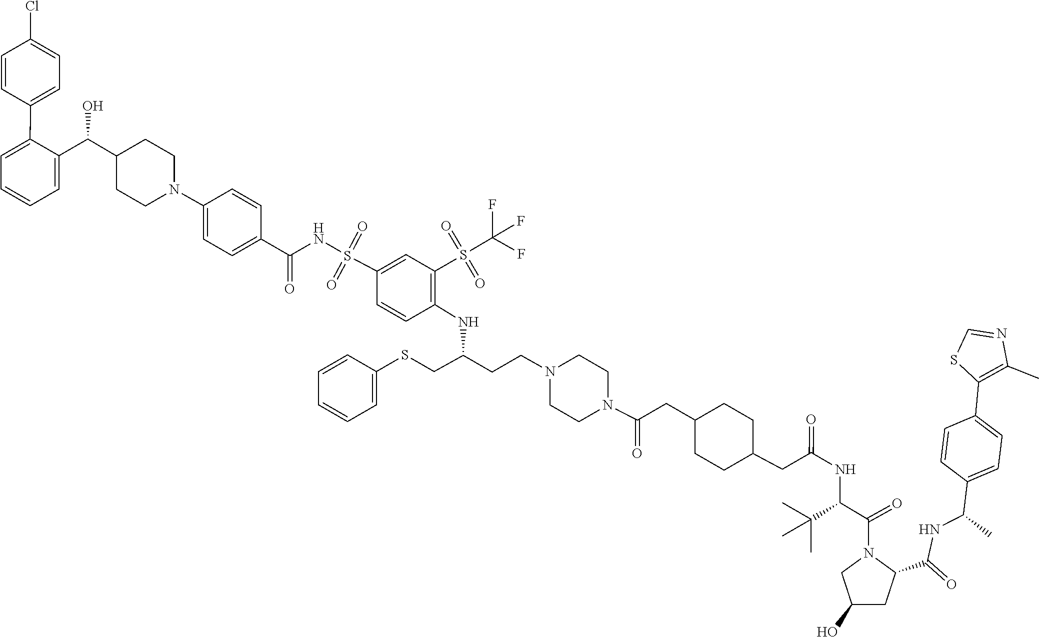 [SMILES: Cc1ncsc1-c1ccc([C@H](C)NC(=O)[C@@H]2C[C@@H](O)CN2C(=O)[C@@H](NC(=O)CC2CCC(CC(=O)N3CCN(CC[C@H](CSc4ccccc4)Nc4ccc(S(=O)(=O)NC(=O)c5ccc(N6CCC([C@@H](O)c7ccccc7-c7ccc(Cl)cc7)CC6)cc5)cc4S(=O)(=O)C(F)(F)F)CC3)CC2)C(C)(C)C)cc1] |
| I-160 | 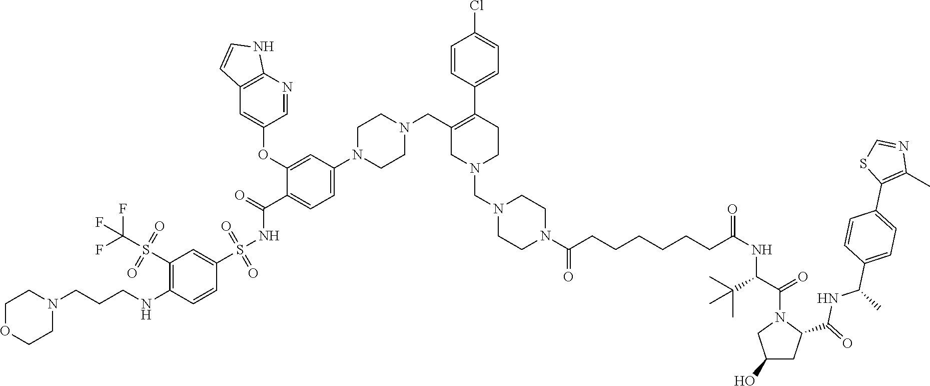 [SMILES: Cc1ncsc1-c1ccc([C@H](C)NC(=O)[C@@H]2C[C@@H](O)CN2C(=O)[C@@H](NC(=O)CCCCCCC(=O)N2CCN(CN3CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-161 | 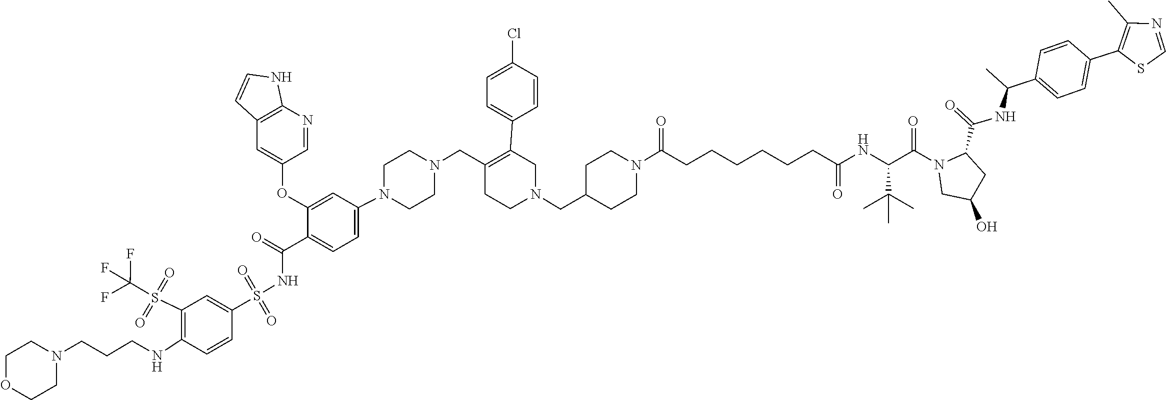 [SMILES: Cc1ncsc1-c1ccc([C@H](C)NC(=O)[C@@H]2C[C@@H](O)CN2C(=O)[C@@H](NC(=O)CCCCCCC(=O)N2CCC(CN3CCC(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)=C(c4ccc(Cl)cc4)C3)CC2)C(C)(C)C)cc1] |
| I-162 | 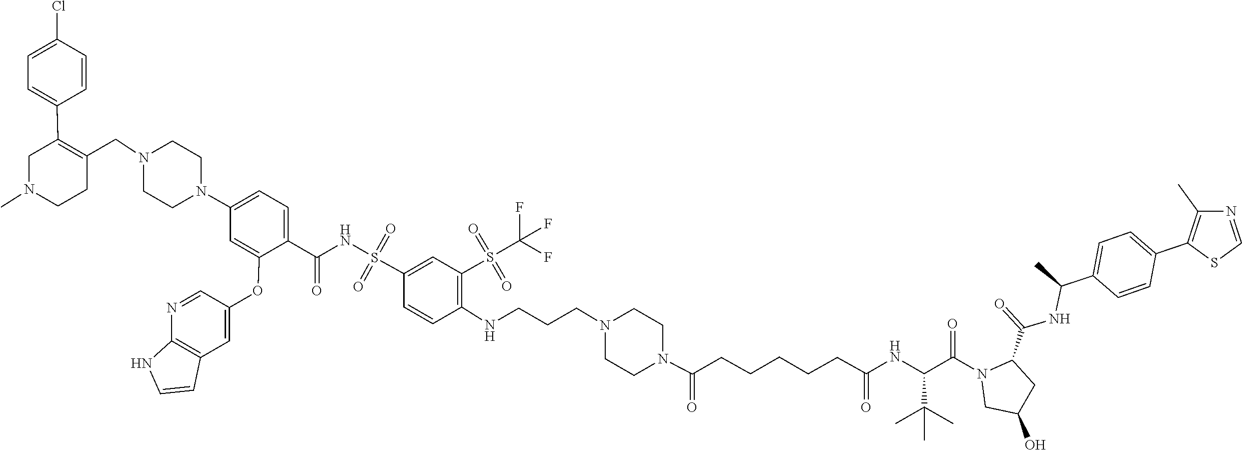 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CN(C)CC6)CC5)cc4Oc4cnc5[nH]ccc5c4)cc3S(=O)(=O)C(F)(F)F)CC2)C(C)(C)C)cc1] |
| I-163 | 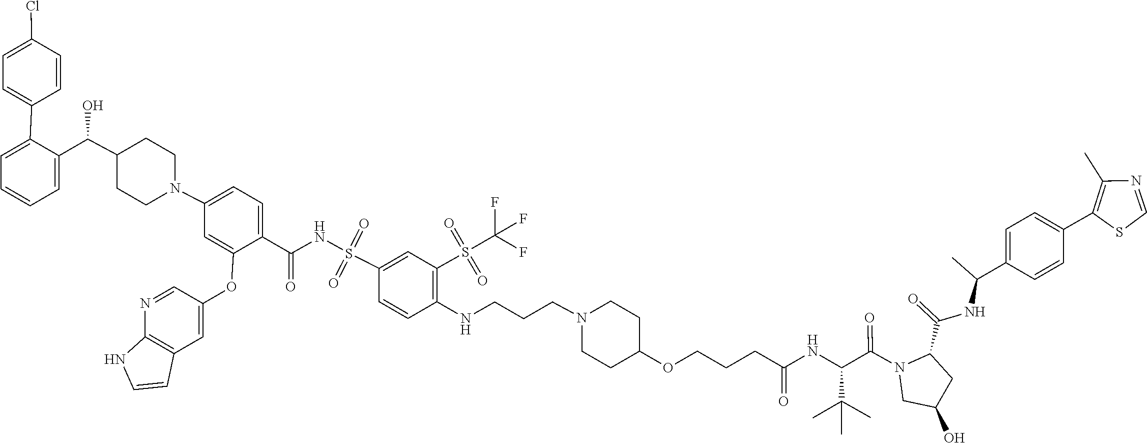 [SMILES: Cc1ncsc1-c1ccc([C@H](C)NC(=O)[C@@H]2C[C@@H](O)CN2C(=O)[C@@H](NC(=O)CCCOC2CCN(CCCNc3ccc(S(=O)(=O)NC(=O)c4ccc(N5CCC([C@@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-164 | 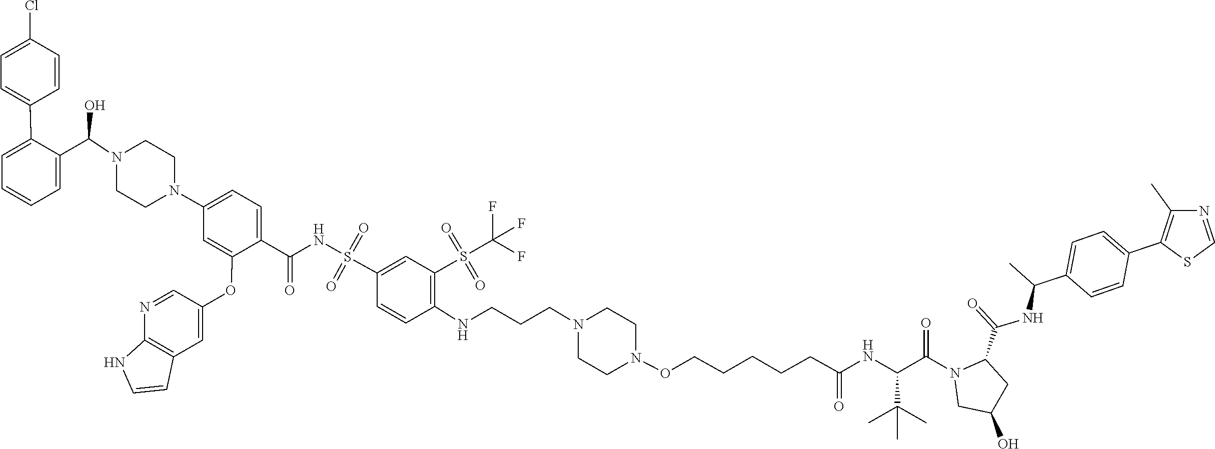 [SMILES: Cc1ncsc1-c1ccc([C@H](C)NC(=O)[C@@H]2C[C@@H](O)CN2C(=O)[C@@H](NC(=O)CCCCCON2CCN(CCCNc3ccc(S(=O)(=O)NC(=O)c4ccc(N5CCN([C@H](O)c6ccccc6-c6ccc(Cl)cc6)CC5)cc4Oc4cnc5[nH]ccc5c4)cc3S(=O)(=O)C(F)(F)F)CC2)C(C)(C)C)cc1] |
| I-165 | 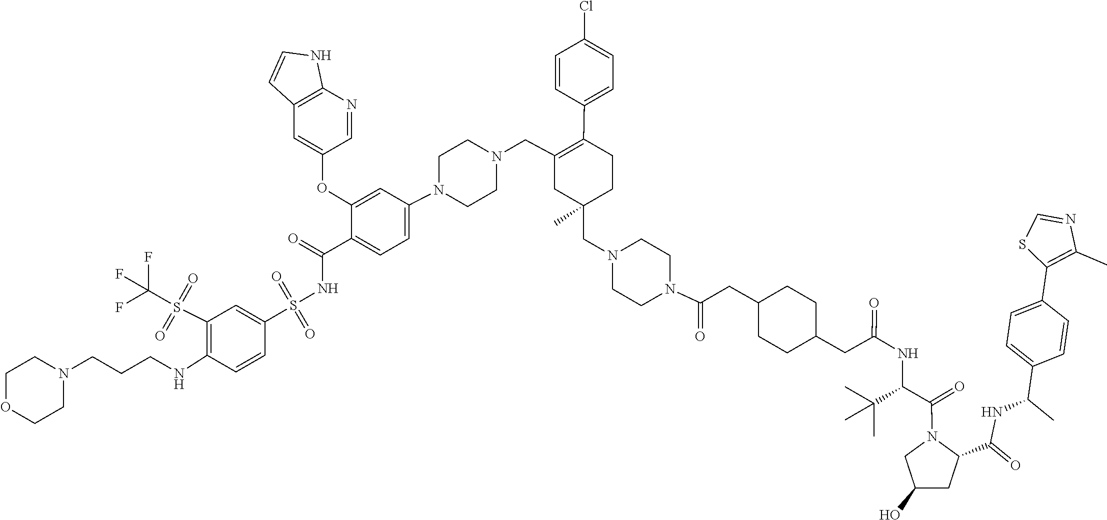 [SMILES: Cc1ncsc1-c1ccc([C@H](C)NC(=O)[C@@H]2C[C@@H](O)CN2C(=O)[C@@H](NC(=O)CC2CCC(CC(=O)N3CCN(C[C@]4(C)CCC(c5ccc(Cl)cc5)=C(CN5CCN(c6ccc(C(=O)NS(=O)(=O)c7ccc(NCCCN8CCOCC8)c(S(=O)(=O)C(F)(F)F)c7)c(Oc7cnc8[nH]ccc8c7)c6)CC5)C4)CC3)CC2)C(C)(C)C)cc1] |
| I-166 | 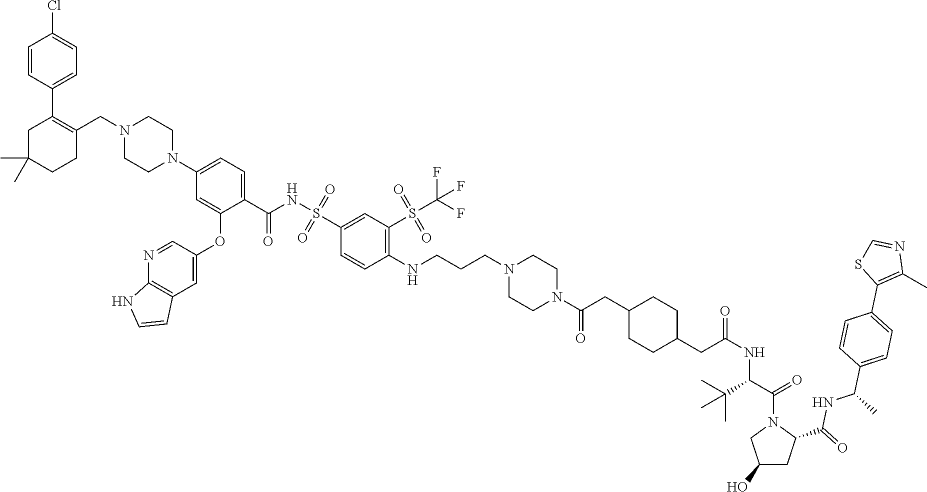 |
| I-167 | 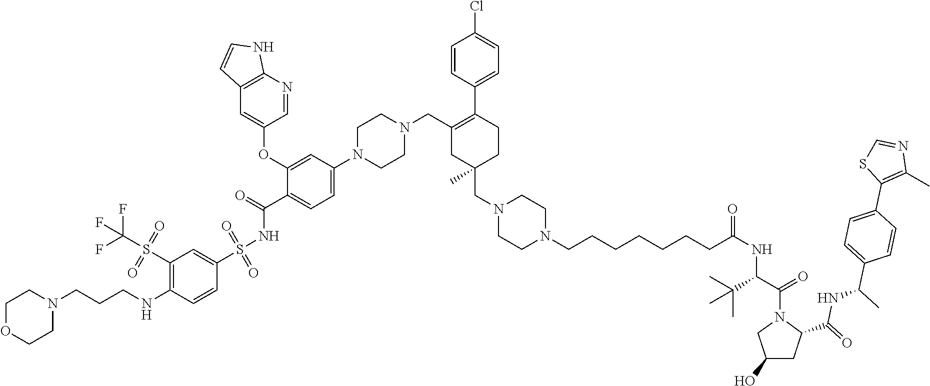 [SMILES: Cc1ncsc1-c1ccc([C@H](C)NC(=O)[C@@H]2C[C@@H](O)CN2C(=O)[C@@H](NC(=O)CCCCCCCN2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-168 | 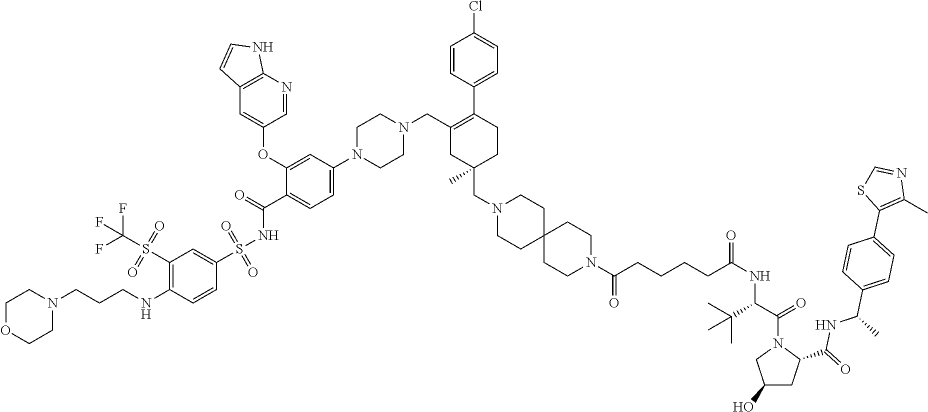 [SMILES: Cc1ncsc1-c1ccc([C@H](C)NC(=O)[C@@H]2C[C@@H](O)CN2C(=O)[C@@H](NC(=O)CCCCC(=O)N2CCC3(CCN(C[C@]4(C)CCC(c5ccc(Cl)cc5)=C(CN5CCN(c6ccc(C(=O)NS(=O)(=O)c7ccc(NCCCN8CCOCC8)c(S(=O)(=O)C(F)(F)F)c7)c(Oc7cnc8[nH]ccc8c7)c6)CC5)C4)CC3)CC2)C(C)(C)C)cc1] |
| I-169 | 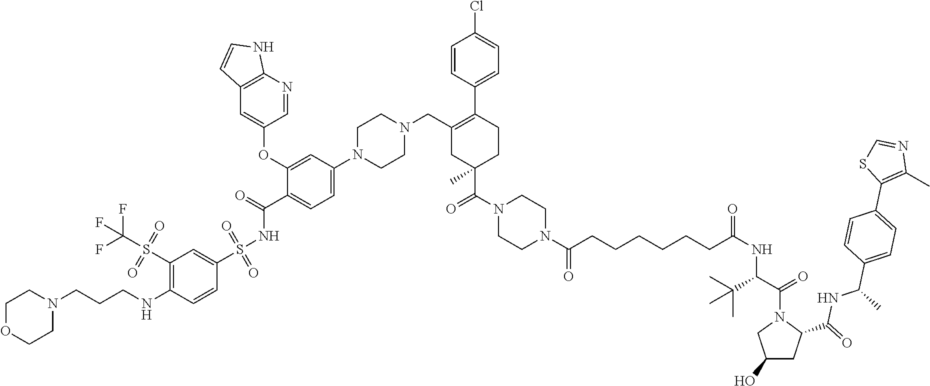 [SMILES: Cc1ncsc1-c1ccc([C@H](C)NC(=O)[C@@H]2C[C@@H](O)CN2C(=O)[C@@H](NC(=O)CCCCCCC(=O)N2CCN(C(=O)[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-170 | 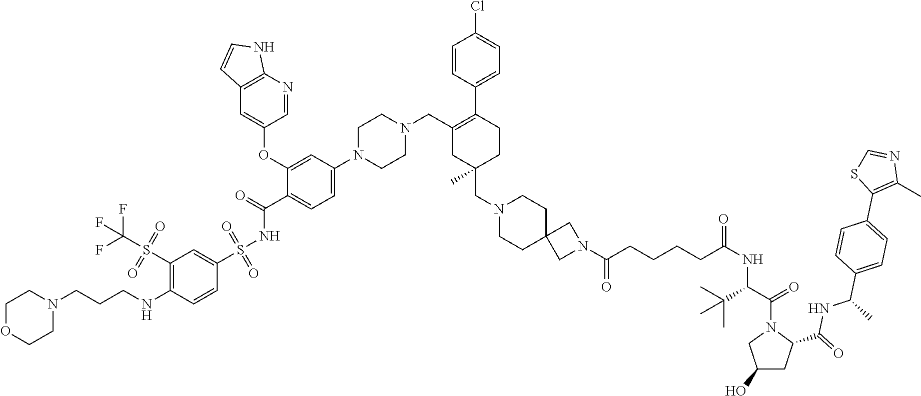 [SMILES: Cc1ncsc1-c1ccc([C@H](C)NC(=O)[C@@H]2C[C@@H](O)CN2C(=O)[C@@H](NC(=O)CCCCC(=O)N2CC3(CCN(C[C@]4(C)CCC(c5ccc(Cl)cc5)=C(CN5CCN(c6ccc(C(=O)NS(=O)(=O)c7ccc(NCCCN8CCOCC8)c(S(=O)(=O)C(F)(F)F)c7)c(Oc7cnc8[nH]ccc8c7)c6)CC5)C4)CC3)C2)C(C)(C)C)cc1] |
| I-171 | 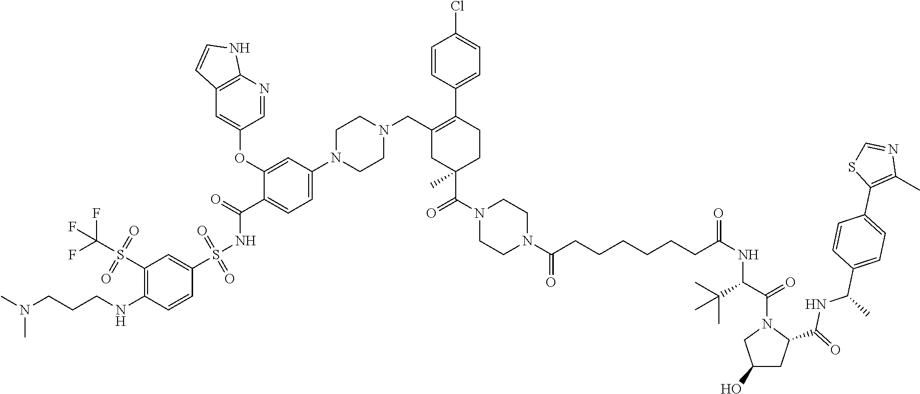 |
| I-172 | 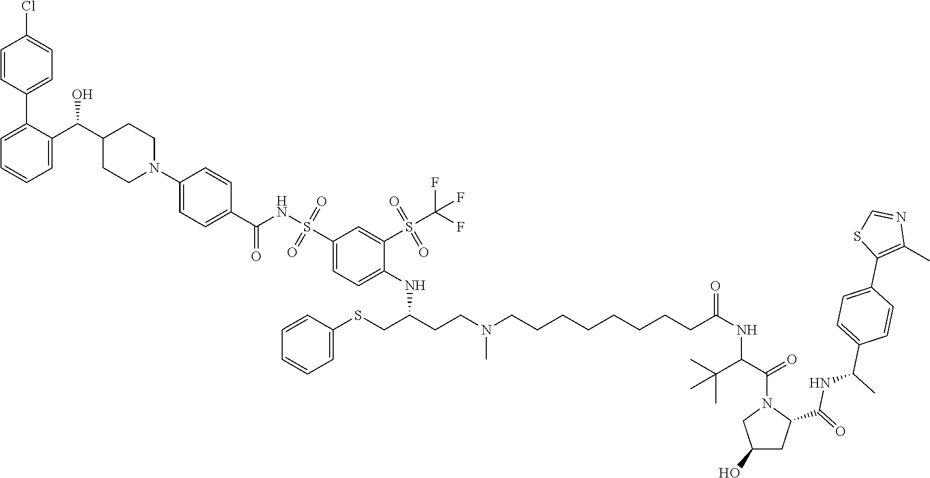 [SMILES: Cc1ncsc1-c1ccc([C@H](C)NC(=O)[C@@H]2C[C@@H](O)CN2C(=O)C(NC(=O)CCCCCCCCN(C)CC[C@H](CSc2ccccc2)Nc2ccc(S(=O)(=O)NC(=O)c3ccc(N4CCC([C@@H](O)c5ccccc5-c5ccc(Cl)cc5)CC4)cc3)cc2S(=O)(=O)C(F)(F)F)C(C)(C)C)cc1] |
| I-173 | 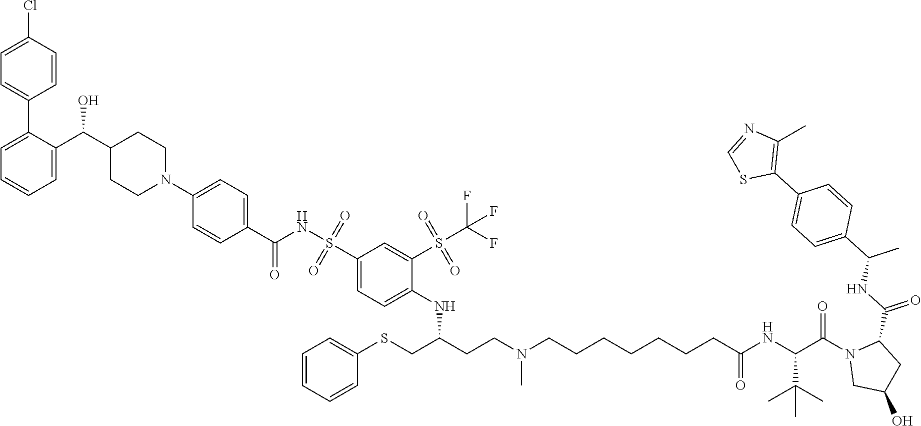 [SMILES: Cc1ncsc1-c1ccc([C@H](C)NC(=O)[C@@H]2C[C@@H](O)CN2C(=O)[C@@H](NC(=O)CCCCCCCN(C)CC[C@H](CSc2ccccc2)Nc2ccc(S(=O)(=O)NC(=O)c3ccc(N4CCC([C@@H](O)c5ccccc5-c5ccc(Cl)cc5)CC4)cc3)cc2S(=O)(=O)C(F)(F)F)C(C)(C)C)cc1] |
| I-174 | 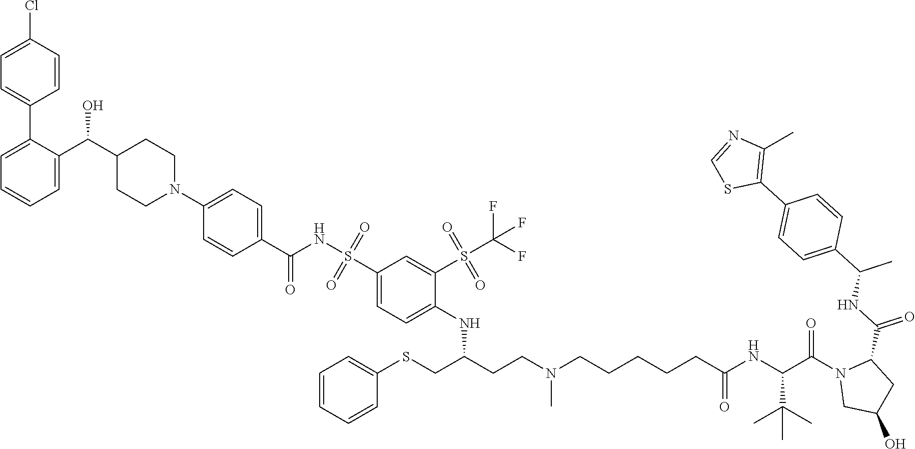 [SMILES: Cc1ncsc1-c1ccc([C@H](C)NC(=O)[C@@H]2C[C@@H](O)CN2C(=O)[C@@H](NC(=O)CCCCCN(C)CC[C@H](CSc2ccccc2)Nc2ccc(S(=O)(=O)NC(=O)c3ccc(N4CCC([C@@H](O)c5ccccc5-c5ccc(Cl)cc5)CC4)cc3)cc2S(=O)(=O)C(F)(F)F)C(C)(C)C)cc1] |
| I-175 | 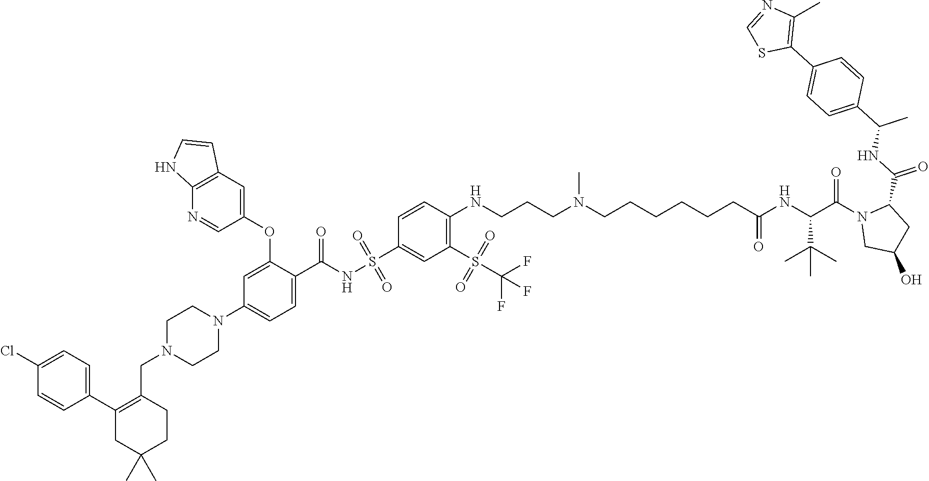 [SMILES: Cc1ncsc1-c1ccc([C@H](C)NC(=O)[C@@H]2C[C@@H](O)CN2C(=O)[C@@H](NC(=O)CCCCCCN(C)CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-176 | 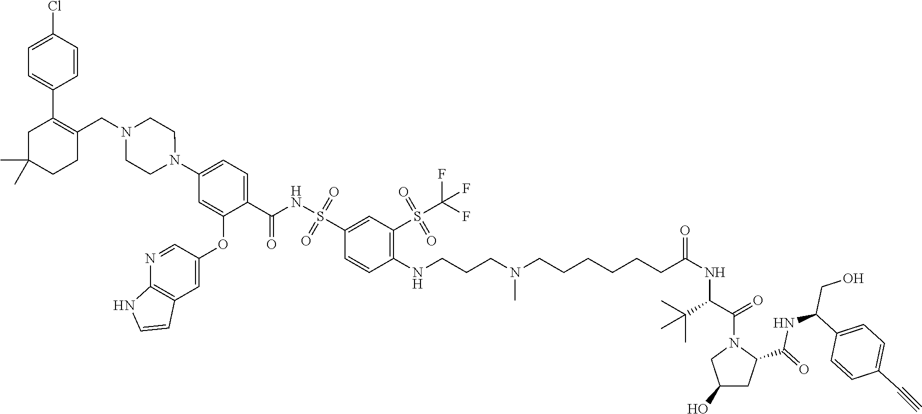 [SMILES: C#Cc1ccc([C@H](CO)NC(=O)[C@@H]2C[C@@H](O)CN2C(=O)[C@@H](NC(=O)CCCCCCN(C)CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-177 | 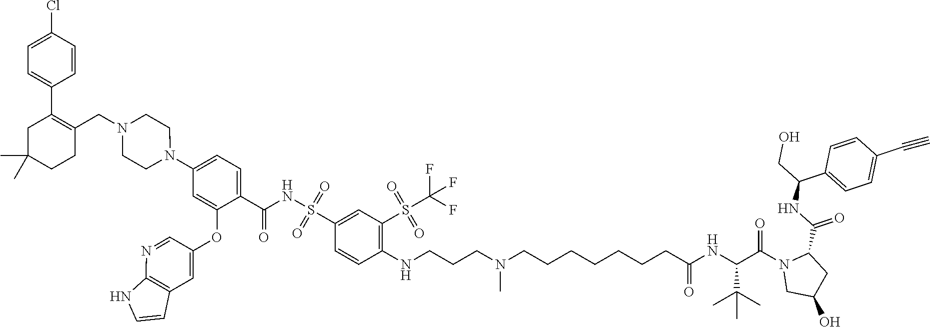 [SMILES: C#Cc1ccc([C@H](CO)NC(=O)[C@@H]2C[C@@H](O)CN2C(=O)[C@@H](NC(=O)CCCCCCCN(C)CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-178 | 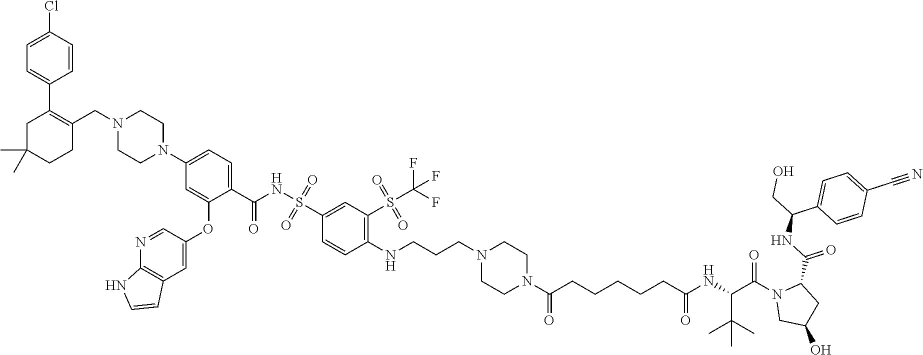 [SMILES: CC1(C)CCC(CN2CCN(c3ccc(C(=O)NS(=O)(=O)c4ccc(NCCCN5CCN(C(=O)CCCCCC(=O)N[C@H](C(=O)N6C[C@H](O)C[C@H]6C(=O)N[C@@H](CO)c6ccc(C#N)cc6)C(C)(C)C)CC5)c(S(=O)(=O)C(F)(F)F)c4)c(Oc4cnc5[nH]ccc5c4)c3)CC2)=C(c2ccc(Cl)cc2)C1] |
| I-179 | 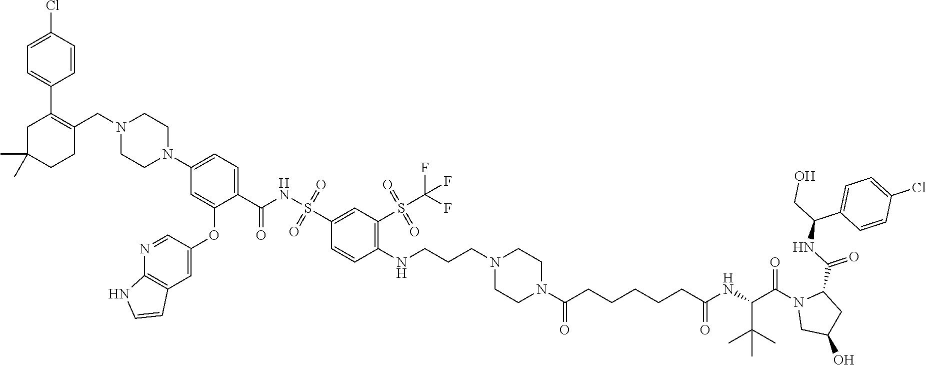 [SMILES: CC1(C)CCC(CN2CCN(c3ccc(C(=O)NS(=O)(=O)c4ccc(NCCCN5CCN(C(=O)CCCCCC(=O)N[C@H](C(=O)N6C[C@H](O)C[C@H]6C(=O)N[C@@H](CO)c6ccc(Cl)cc6)C(C)(C)C)CC5)c(S(=O)(=O)C(F)(F)F)c4)c(Oc4cnc5[nH]ccc5c4)c3)CC2)=C(c2ccc(Cl)cc2)C1] |
| I-180 | 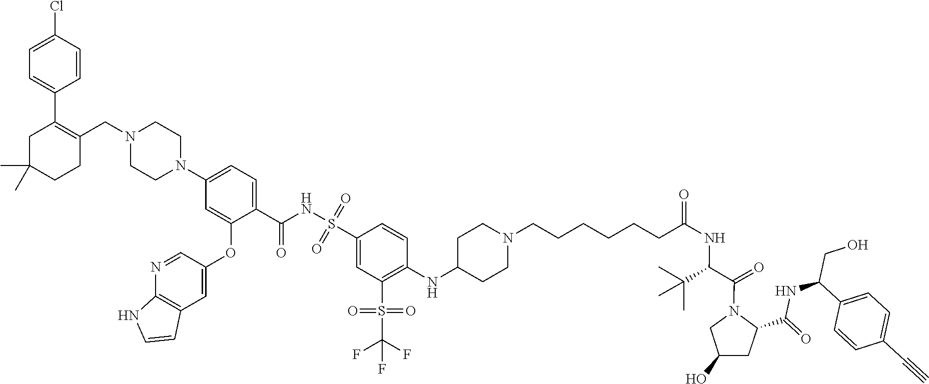 [SMILES: C#Cc1ccc([C@H](CO)NC(=O)[C@@H]2C[C@@H](O)CN2C(=O)[C@@H](NC(=O)CCCCCCN2CCC(Nc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-181 | 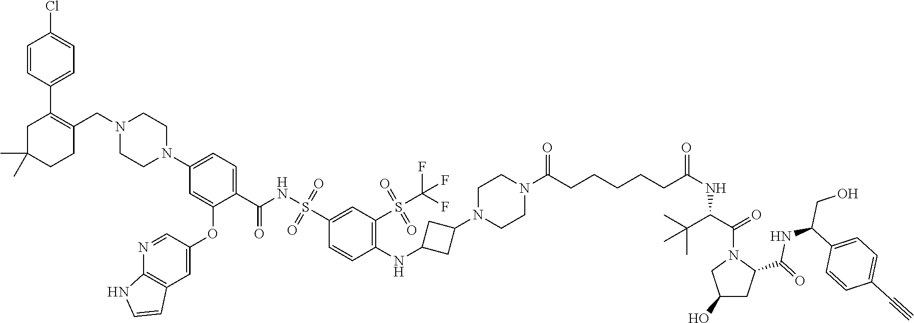 [SMILES: C#Cc1ccc([C@H](CO)NC(=O)[C@@H]2C[C@@H](O)CN2C(=O)[C@@H](NC(=O)CCCCCC(=O)N2CCN(C3CC(Nc4ccc(S(=O)(=O)NC(=O)c5ccc(N6CCN(CC7=C(c8ccc(Cl)cc8)CC(C)(C)CC7)CC6)cc5Oc5cnc6[nH]ccc6c5)cc4S(=O)(=O)C(F)(F)F)C3)CC2)C(C)(C)C)cc1] |
| I-182 | 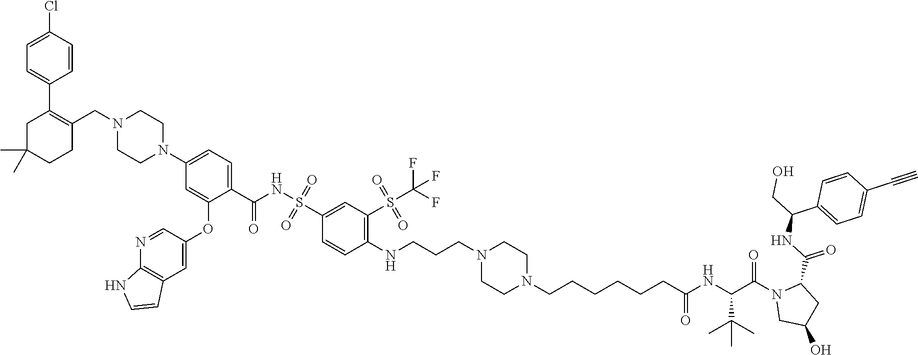 [SMILES: C#Cc1ccc([C@H](CO)NC(=O)[C@@H]2C[C@@H](O)CN2C(=O)[C@@H](NC(=O)CCCCCCN2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-183 | 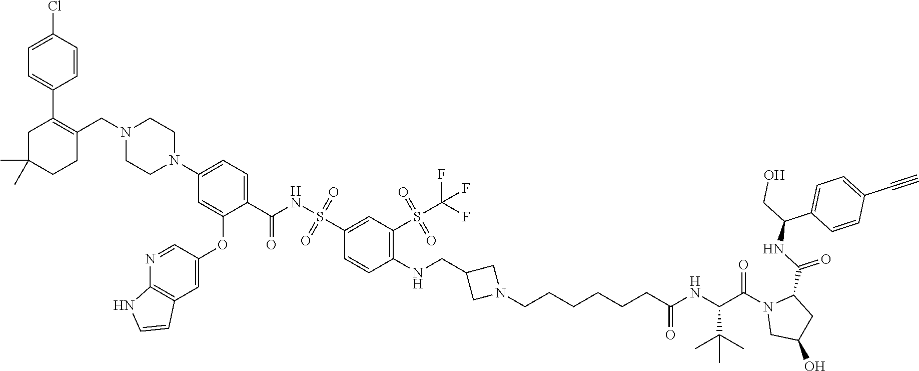 [SMILES: C#Cc1ccc([C@H](CO)NC(=O)[C@@H]2C[C@@H](O)CN2C(=O)[C@@H](NC(=O)CCCCCCN2CC(CNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)C2)C(C)(C)C)cc1] |
| I-184 | 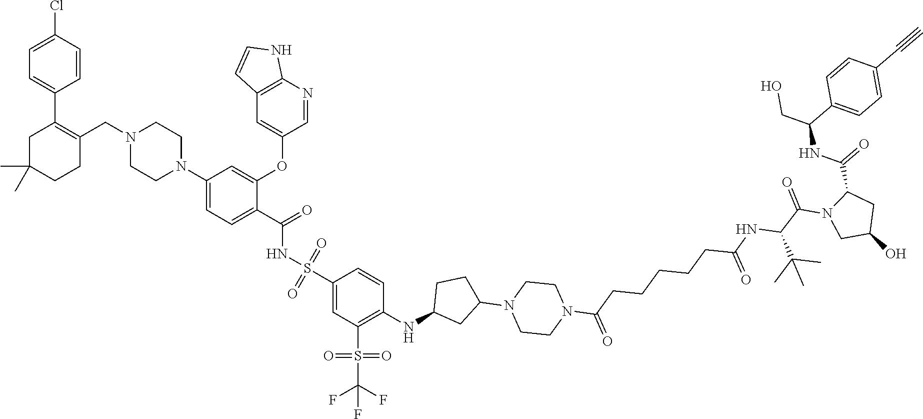 [SMILES: C#Cc1ccc([C@H](CO)NC(=O)[C@@H]2C[C@@H](O)CN2C(=O)[C@@H](NC(=O)CCCCCC(=O)N2CCN(C3CC[C@H](Nc4ccc(S(=O)(=O)NC(=O)c5ccc(N6CCN(CC7=C(c8ccc(Cl)cc8)CC(C)(C)CC7)CC6)cc5Oc5cnc6[nH]ccc6c5)cc4S(=O)(=O)C(F)(F)F)C3)CC2)C(C)(C)C)cc1] |
| I-185 | 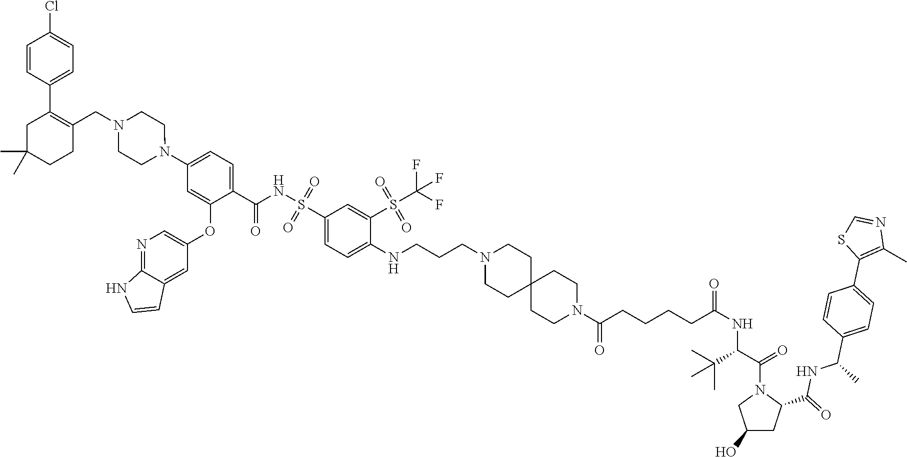 |
| I-186 | 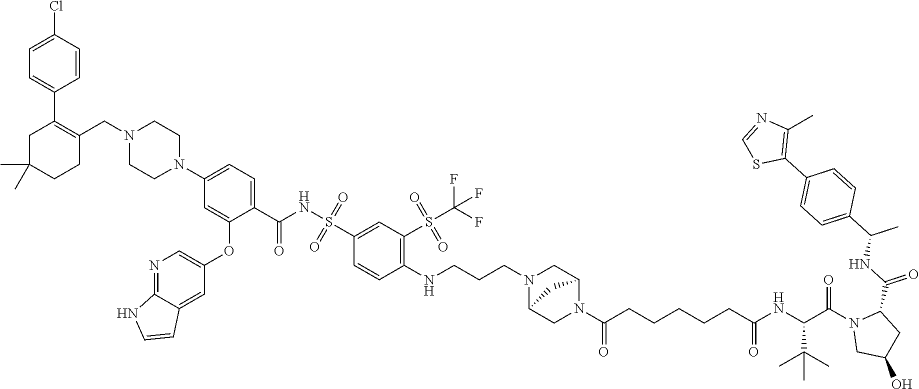 [SMILES: Cc1ncsc1-c1ccc([C@H](C)NC(=O)[C@@H]2C[C@@H](O)CN2C(=O)[C@@H](NC(=O)CCCCCC(=O)N2C[C@@H]3C[C@H]2CN3CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-187 | 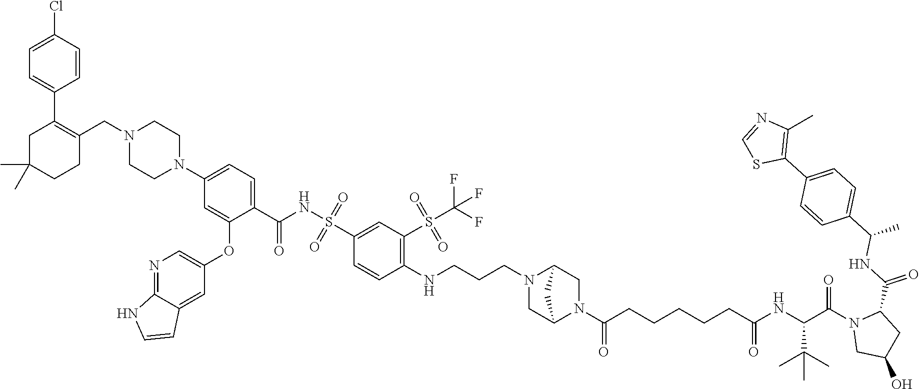 [SMILES: Cc1ncsc1-c1ccc([C@H](C)NC(=O)[C@@H]2C[C@@H](O)CN2C(=O)[C@@H](NC(=O)CCCCCC(=O)N2C[C@H]3C[C@@H]2CN3CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C(C)(C)C)cc1] |
| I-188 | 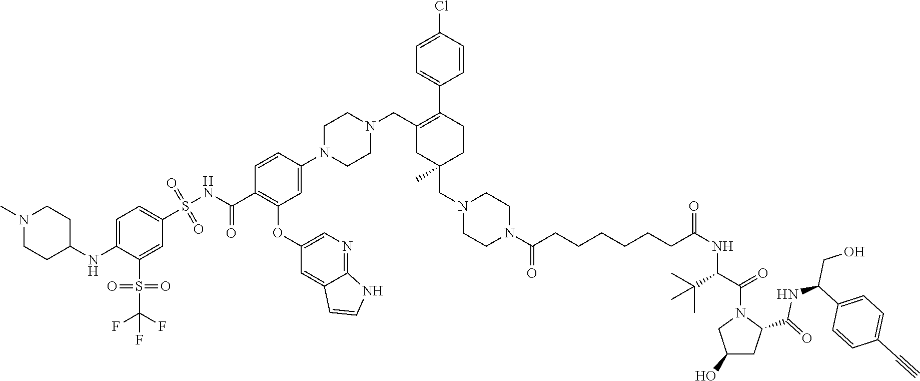 [SMILES: C#Cc1ccc([C@H](CO)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NC7CCN(C)CC7)c(S(=O)(=O)C(F)(F)F)c6)c(Oc6cnc7[nH]ccc7c6)c5)CC4)C3)CC2)C(C)(C)C)cc1] |
| I-189 | 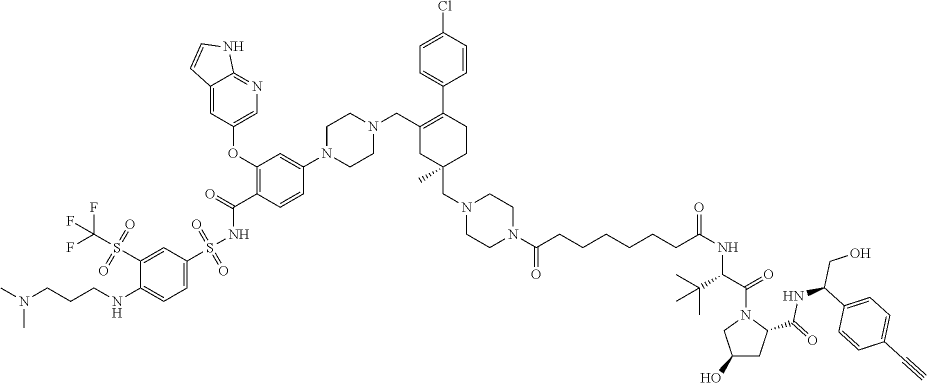 [SMILES: C#Cc1ccc([C@H](CO)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NCCCN(C)C)c(S(=O)(=O)C(F)(F)F)c6)c(Oc6cnc7[nH]ccc7c6)c5)CC4)C3)CC2)C(C)(C)C)cc1] |
| I-190 | 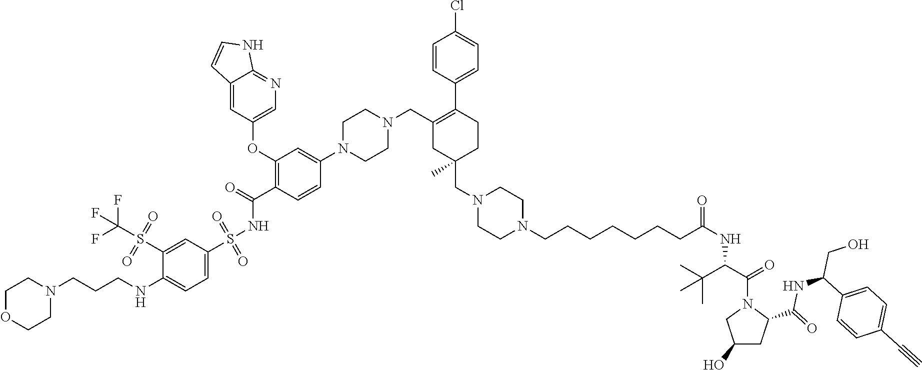 [SMILES: C#Cc1ccc([C@H](CO)NC(=O)[C@@H]2C[C@@H](O)CN2C(=O)[C@@H](NC(=O)CCCCCCCN2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-191 | 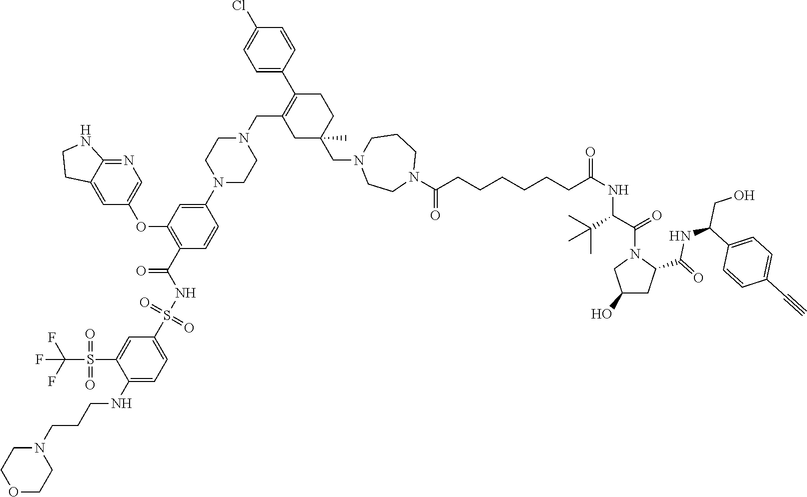 [SMILES: C#Cc1ccc([C@H](CO)NC(=O)[C@@H]2C[C@@H](O)CN2C(=O)[C@@H](NC(=O)CCCCCCC(=O)N2CCCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7c(c6)CCN7)c5)CC4)C3)CC2)C(C)(C)C)cc1] |
| I-192 | 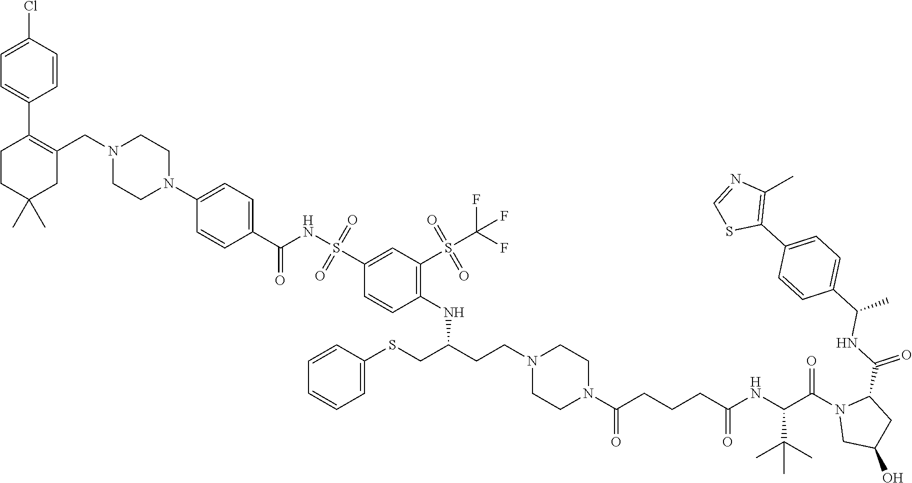 [SMILES: Cc1ncsc1-c1ccc([C@H](C)NC(=O)[C@@H]2C[C@@H](O)CN2C(=O)[C@@H](NC(=O)CCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1] |
| I-193 | 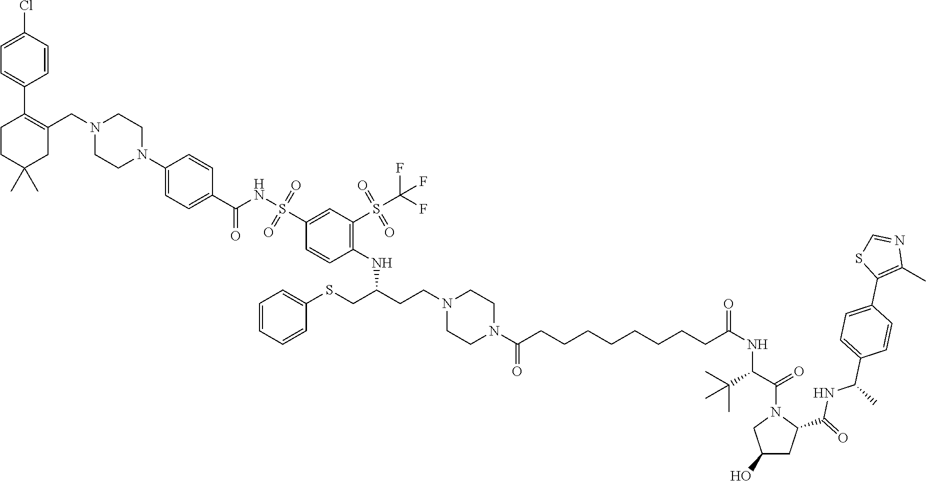 [SMILES: Cc1ncsc1-c1ccc([C@H](C)NC(=O)[C@@H]2C[C@@H](O)CN2C(=O)[C@@H](NC(=O)CCCCCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1] |
| I-194 | 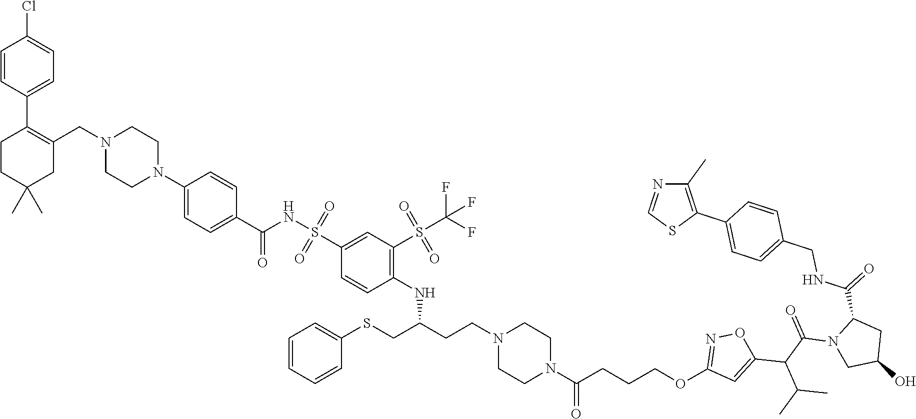 [SMILES: Cc1ncsc1-c1ccc(CNC(=O)[C@@H]2C[C@@H](O)CN2C(=O)C(c2cc(OCCCC(=O)N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)no2)C(C)C)cc1] |
| I-195 | 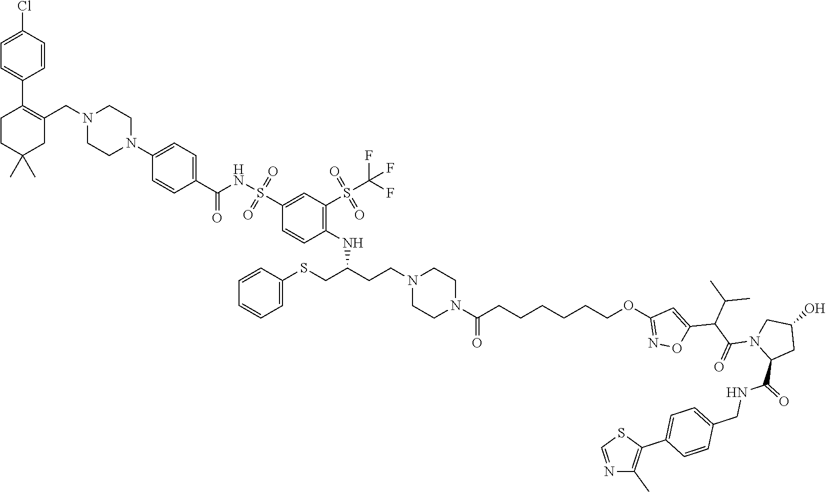 [SMILES: Cc1ncsc1-c1ccc(CNC(=O)[C@@H]2C[C@@H](O)CN2C(=O)C(c2cc(OCCCCCCC(=O)N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)no2)C(C)C)cc1] |
| I-196 | 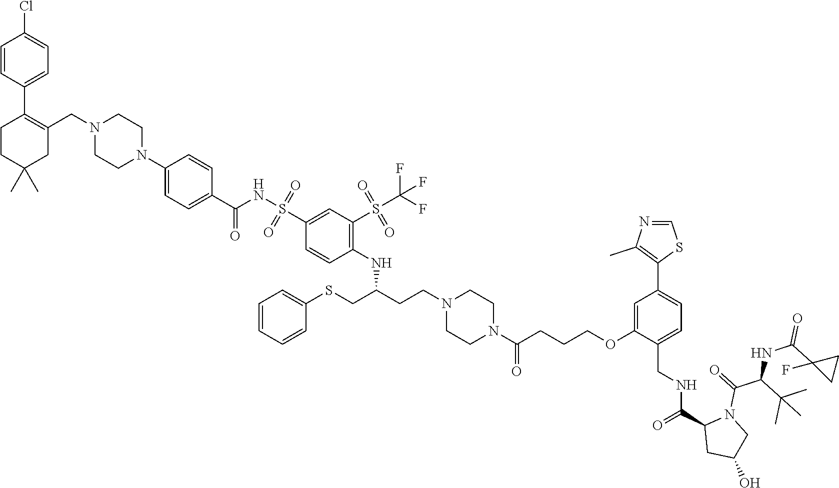 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-197 | 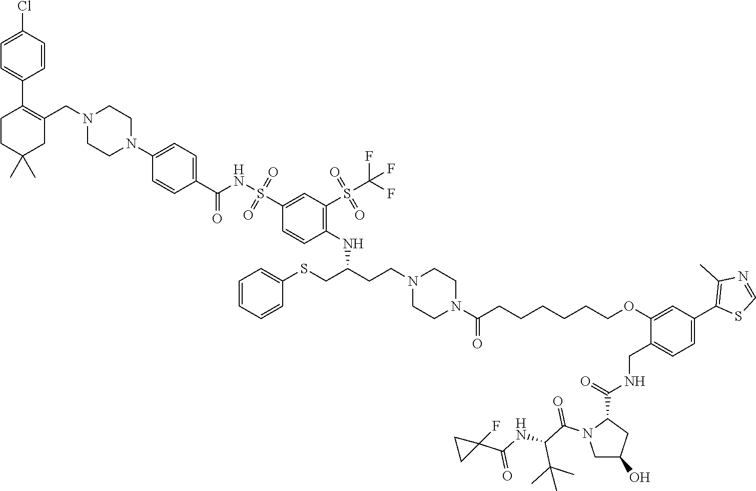 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-198 | 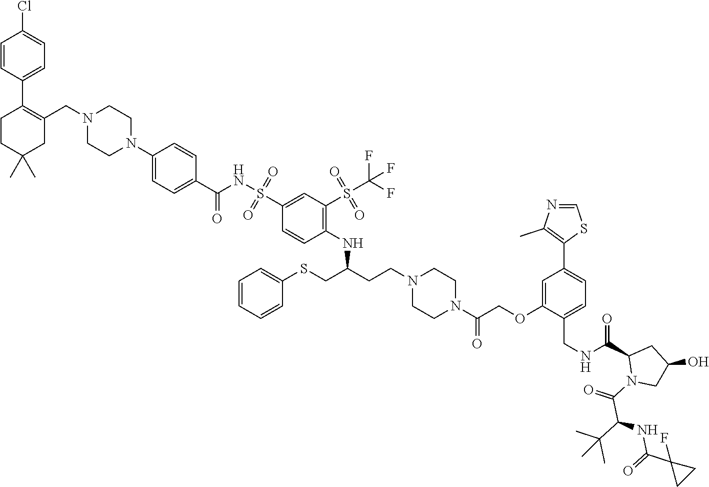 [SMILES: Cc1ncsc1-c1ccc(CNC(=O)[C@H]2C[C@@H](O)CN2C(=O)[C@@H](NC(=O)C2(F)CC2)C(C)(C)C)c(OCC(=O)N2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-199 | 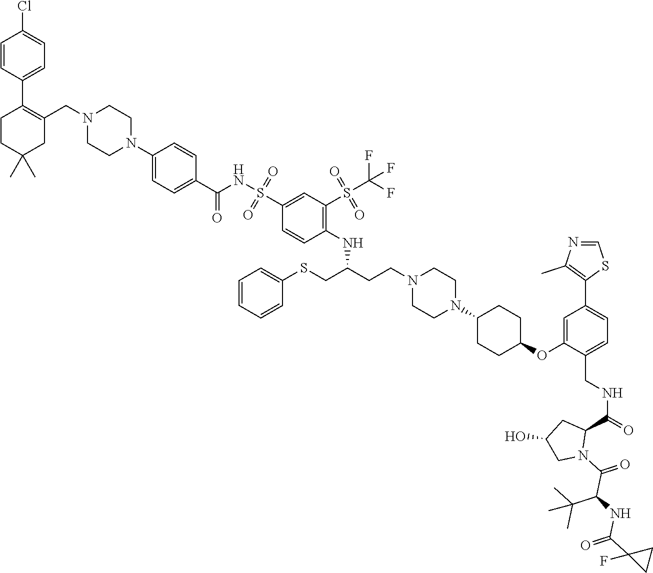 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@H](N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)c1] |
| I-200 | 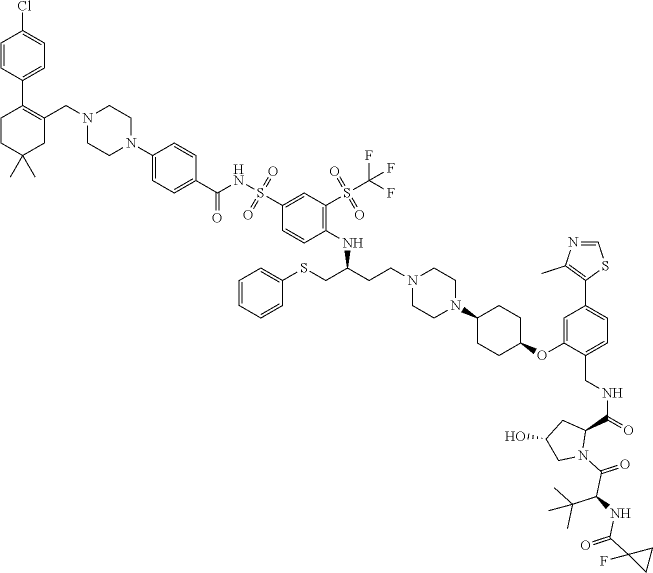 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@@H](N3CCN(CC[C@@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)c1] |
| I-201 | 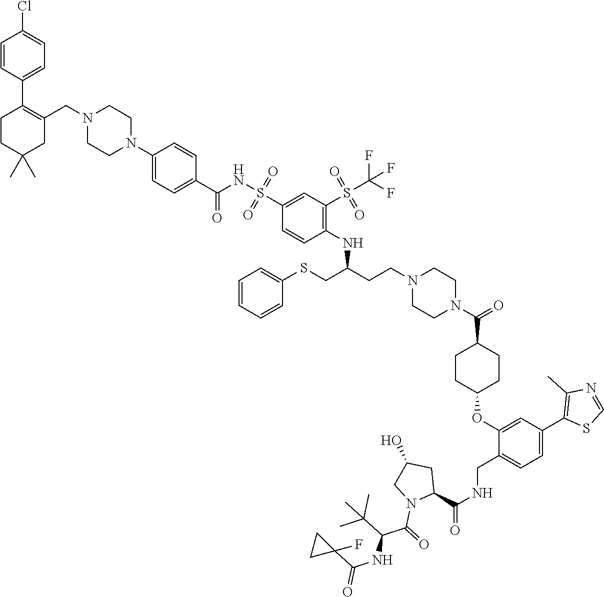 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@H](C(=O)N3CCN(CC[C@@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)c1] |
| I-202 | 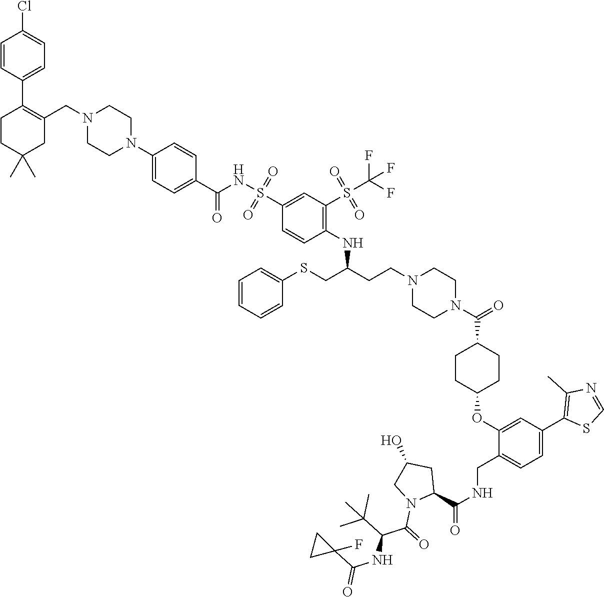 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2CC[C@@H](C(=O)N3CCN(CC[C@@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)c1] |
| I-203 | 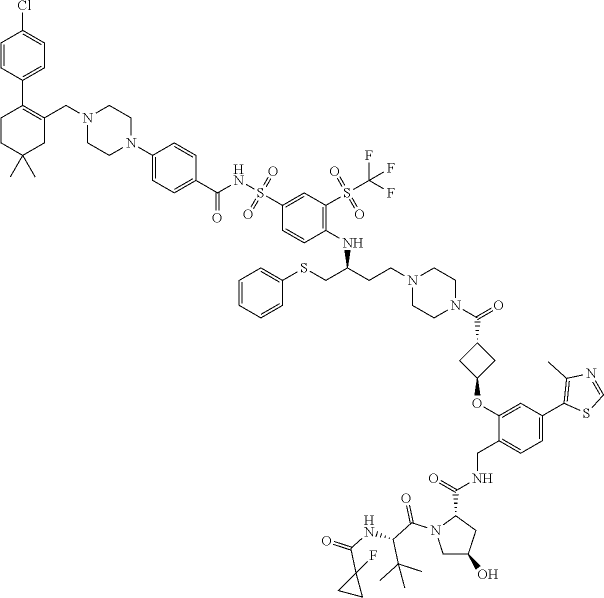 [SMILES: Cc1ncsc1-c1ccc(CNC(=O)[C@@H]2C[C@@H](O)CN2C(=O)[C@@H](NC(=O)C2(F)CC2)C(C)(C)C)c(O[C@H]2C[C@H](C(=O)N3CCN(CC[C@@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)C2)c1] |
| I-204 | 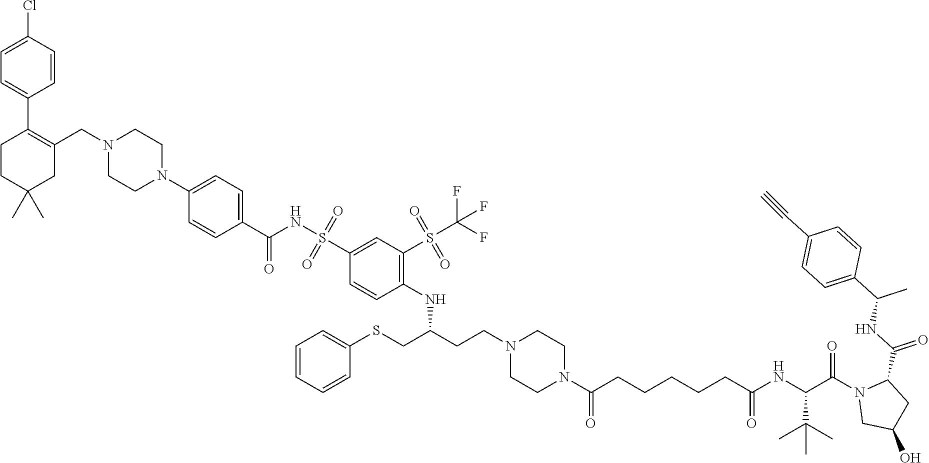 [SMILES: C#Cc1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1] |
| I-205 | 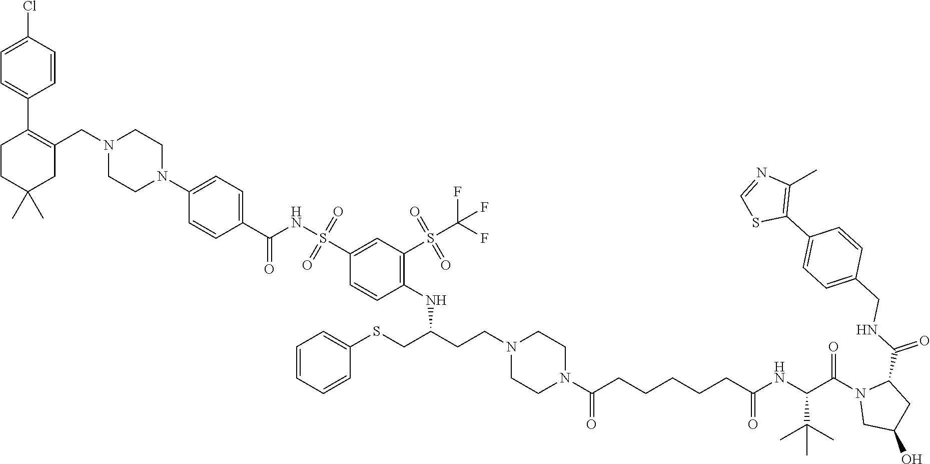 [SMILES: Cc1ncsc1-c1ccc(CNC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1] |
| I-206 | 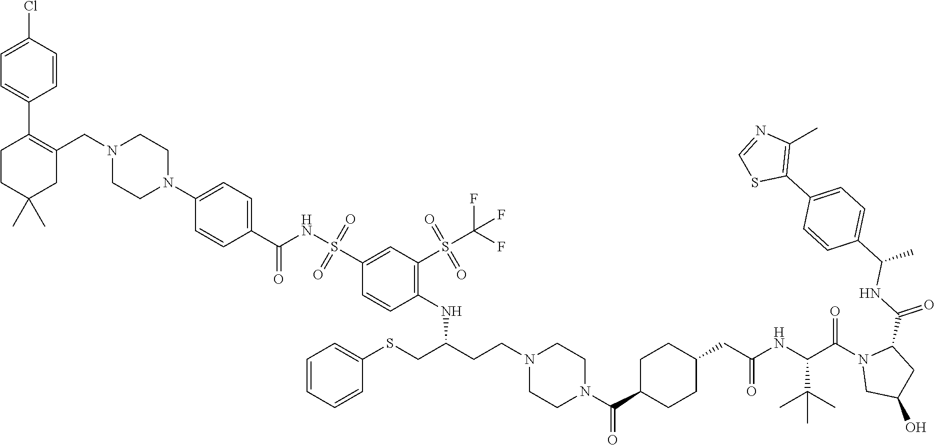 [SMILES: Cc1ncsc1-c1ccc([C@H](C)NC(=O)[C@@H]2C[C@@H](O)CN2C(=O)[C@@H](NC(=O)C[C@H]2CC[C@H](C(=O)N3CCN(CC[C@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)C(C)(C)C)cc1] |
| I-207 | 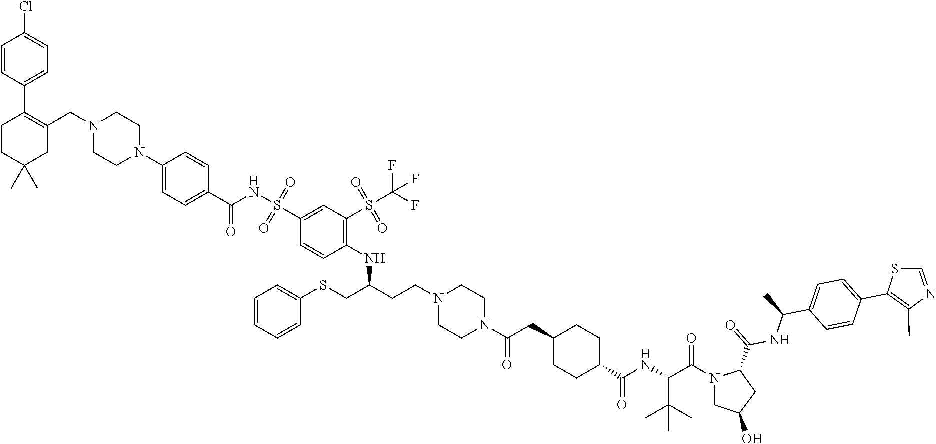 [SMILES: Cc1ncsc1-c1ccc([C@H](C)NC(=O)[C@@H]2C[C@@H](O)CN2C(=O)[C@@H](NC(=O)[C@H]2CC[C@H](CC(=O)N3CCN(CC[C@@H](CSc4ccccc4)Nc4ccc(S(=O)(=O)NC(=O)c5ccc(N6CCN(CC7=C(c8ccc(Cl)cc8)CCC(C)(C)C7)CC6)cc5)cc4S(=O)(=O)C(F)(F)F)CC3)CC2)C(C)(C)C)cc1] |
| I-208 | 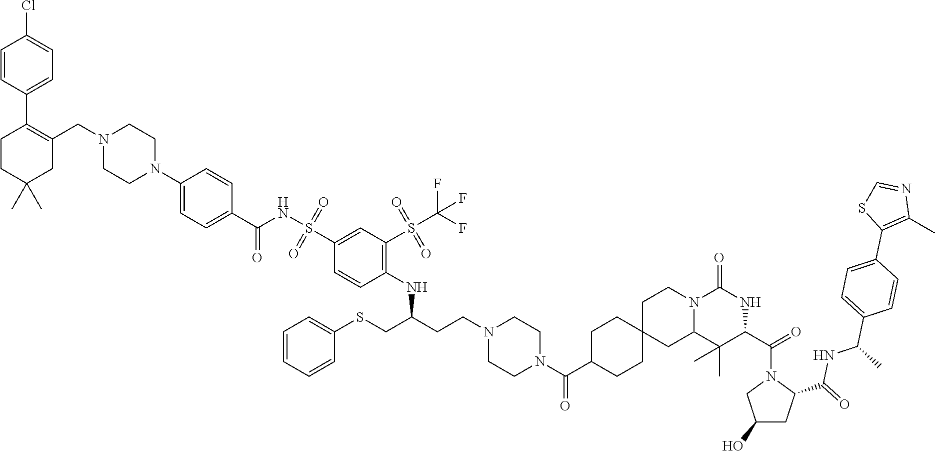 |
| I-209 | 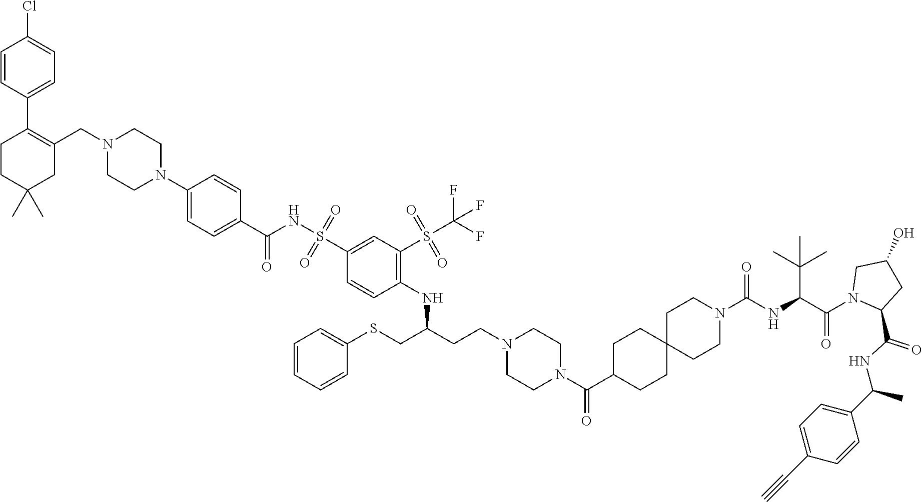 [SMILES: C#Cc1ccc([C@H](C)NC(=O)[C@@H]2C[C@@H](O)CN2C(=O)[C@@H](NC(=O)N2CCC3(CCC(C(=O)N4CCN(CC[C@@H](CSc5ccccc5)Nc5ccc(S(=O)(=O)NC(=O)c6ccc(N7CCN(CC8=C(c9ccc(Cl)cc9)CCC(C)(C)C8)CC7)cc6)cc5S(=O)(=O)C(F)(F)F)CC4)CC3)CC2)C(C)(C)C)cc1] |
| I-210 | 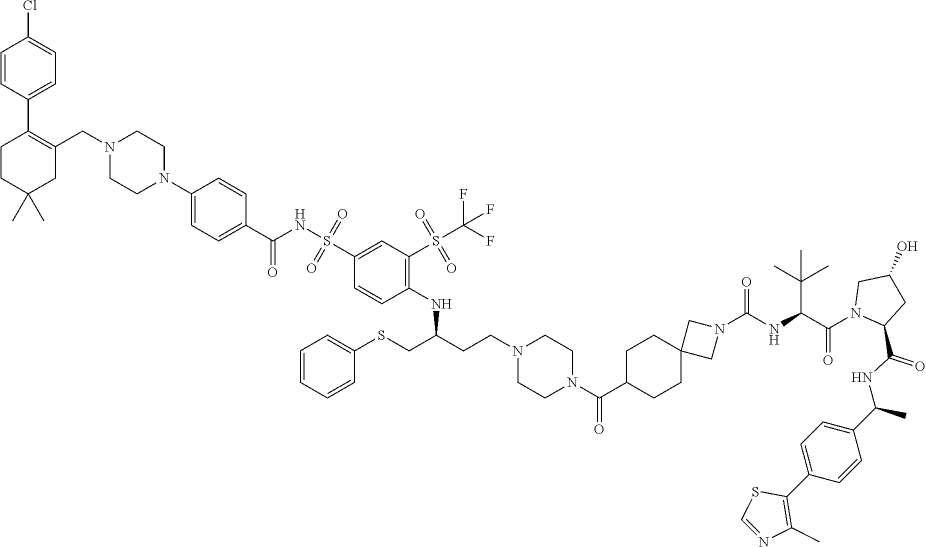 [SMILES: Cc1ncsc1-c1ccc([C@H](C)NC(=O)[C@@H]2C[C@@H](O)CN2C(=O)[C@@H](NC(=O)N2CC3(CCC(C(=O)N4CCN(CC[C@@H](CSc5ccccc5)Nc5ccc(S(=O)(=O)NC(=O)c6ccc(N7CCN(CC8=C(c9ccc(Cl)cc9)CCC(C)(C)C8)CC7)cc6)cc5S(=O)(=O)C(F)(F)F)CC4)CC3)C2)C(C)(C)C)cc1] |
| I-211 | 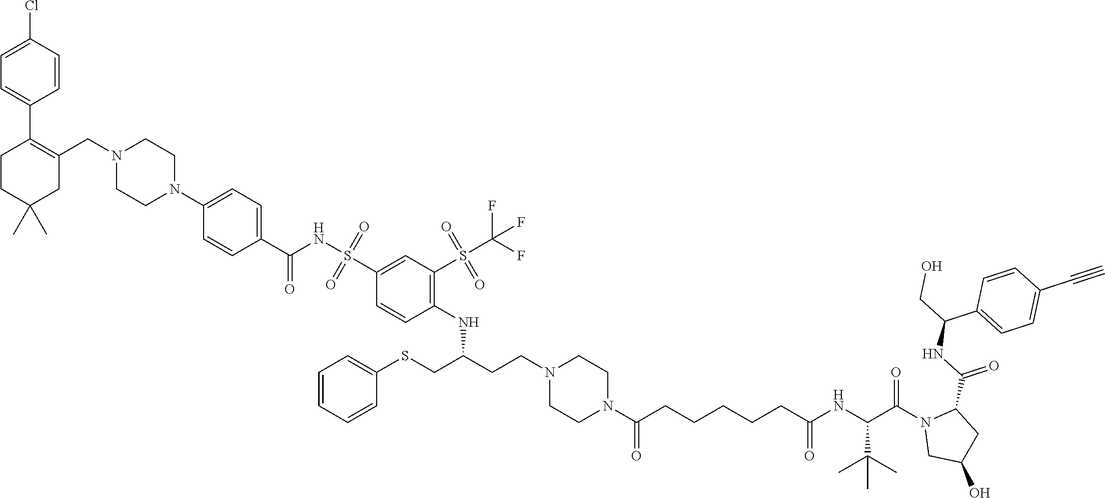 [SMILES: C#Cc1ccc([C@H](CO)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)(C)C)cc1] |
| I-212 | 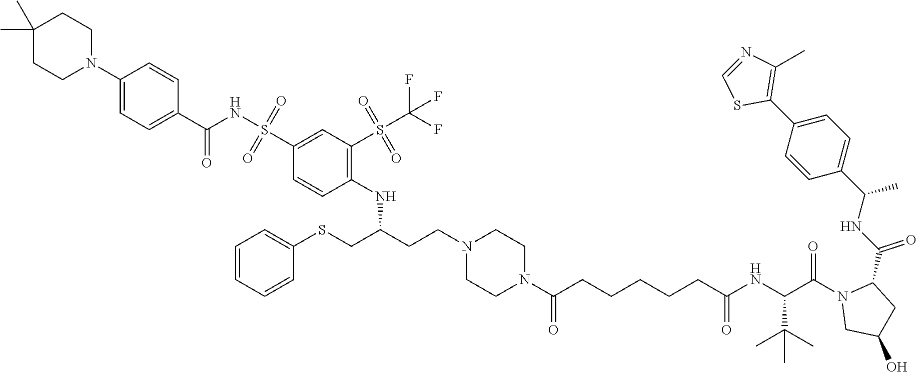 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@H](CSc3ccccc3)Nc3ccc(S(=O)(=O)NC(=O)c4ccc(N5CCC(C)(C)CC5)cc4)cc3S(=O)(=O)C(F)(F)F)CC2)C(C)(C)C)cc1] |
| I-213 | 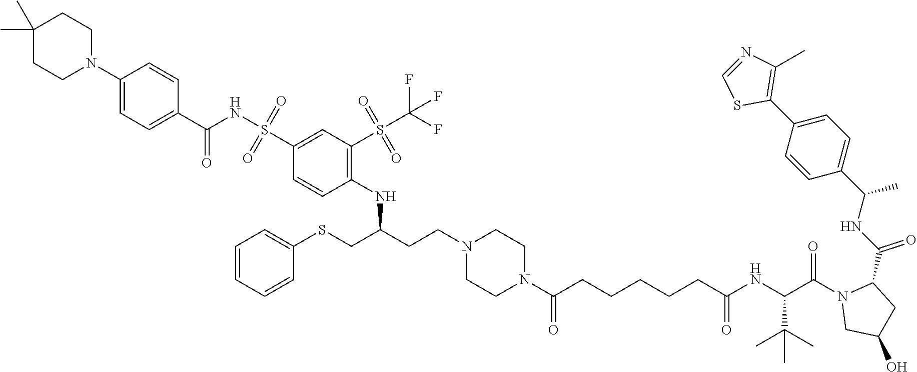 [SMILES: Cc1ncsc1-c1ccc([C@H](C)NC(=O)[C@@H]2C[C@@H](O)CN2C(=O)[C@@H](NC(=O)CCCCCC(=O)N2CCN(CC[C@@H](CSc3ccccc3)Nc3ccc(S(=O)(=O)NC(=O)c4ccc(N5CCC(C)(C)CC5)cc4)cc3S(=O)(=O)C(F)(F)F)CC2)C(C)(C)C)cc1] |
| I-214 | 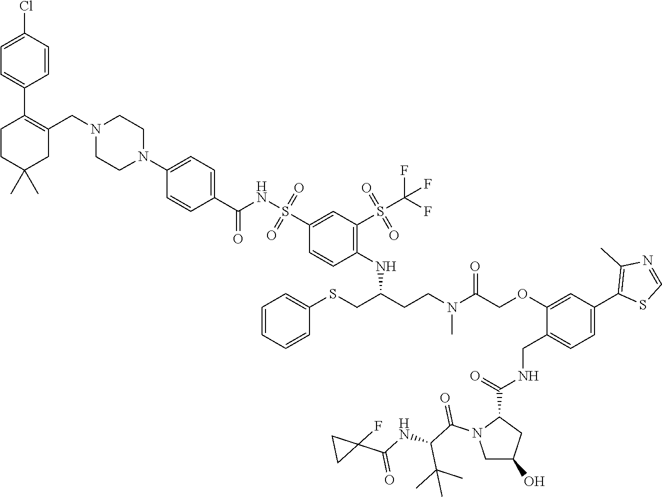 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(=O)N(C)CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)c1] |
| I-215 | 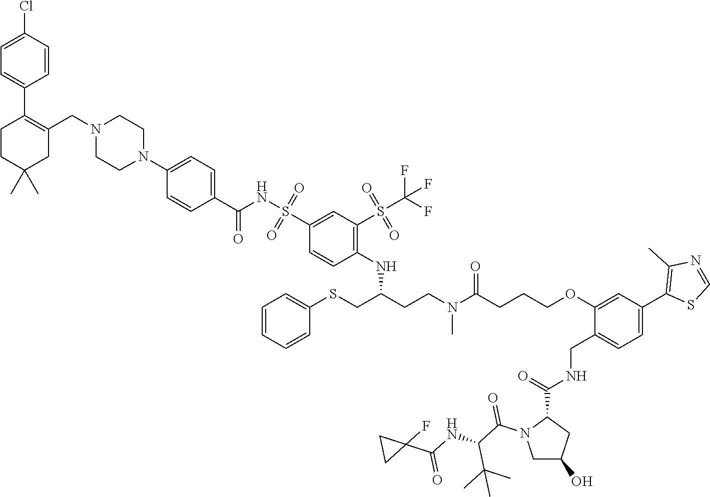 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCC(=O)N(C)CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)c1] |
| I-216 | 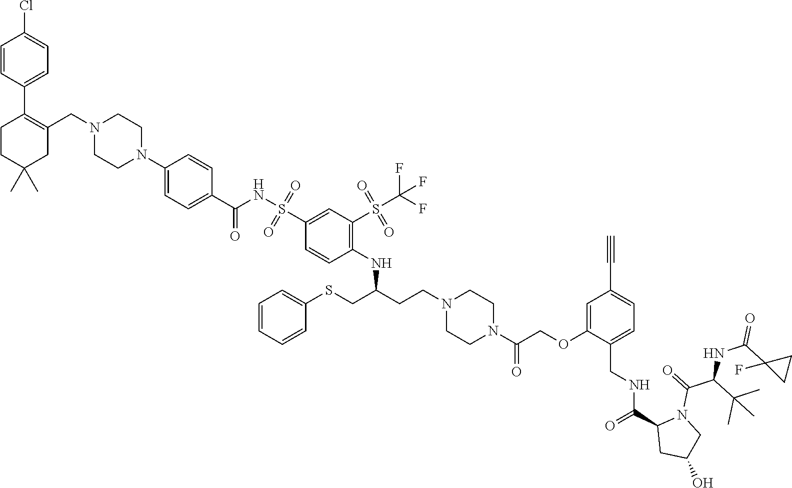 [SMILES: C#Cc1ccc(CNC(=O)[C@@H]2C[C@@H](O)CN2C(=O)[C@@H](NC(=O)C2(F)CC2)C(C)(C)C)c(OCC(=O)N2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-217 | 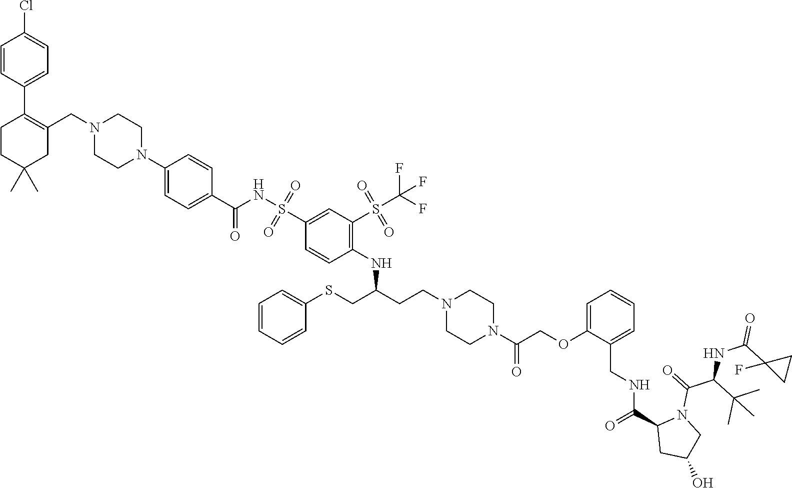 [SMILES: CC1(C)CCC(c2ccc(Cl)cc2)=C(CN2CCN(c3ccc(C(=O)NS(=O)(=O)c4ccc(N[C@@H](CCN5CCN(C(=O)COc6ccccc6CNC(=O)[C@@H]6C[C@@H](O)CN6C(=O)[C@@H](NC(=O)C6(F)CC6)C(C)(C)C)CC5)CSc5ccccc5)c(S(=O)(=O)C(F)(F)F)c4)cc3)CC2)C1] |
| I-218 | 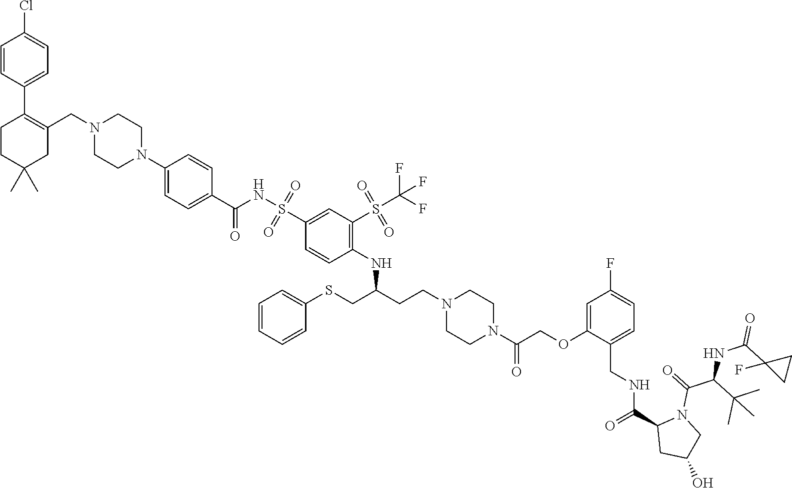 [SMILES: CC1(C)CCC(c2ccc(Cl)cc2)=C(CN2CCN(c3ccc(C(=O)NS(=O)(=O)c4ccc(N[C@@H](CCN5CCN(C(=O)COc6cc(F)ccc6CNC(=O)[C@@H]6C[C@@H](O)CN6C(=O)[C@@H](NC(=O)C6(F)CC6)C(C)(C)C)CC5)CSc5ccccc5)c(S(=O)(=O)C(F)(F)F)c4)cc3)CC2)C1] |
| I-219 | 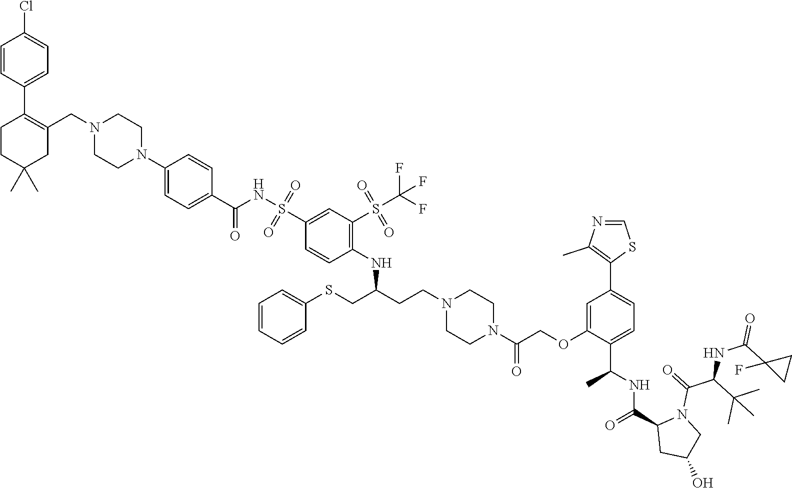 [SMILES: Cc1ncsc1-c1ccc([C@H](C)NC(=O)[C@@H]2C[C@@H](O)CN2C(=O)[C@@H](NC(=O)C2(F)CC2)C(C)(C)C)c(OCC(=O)N2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-220 | 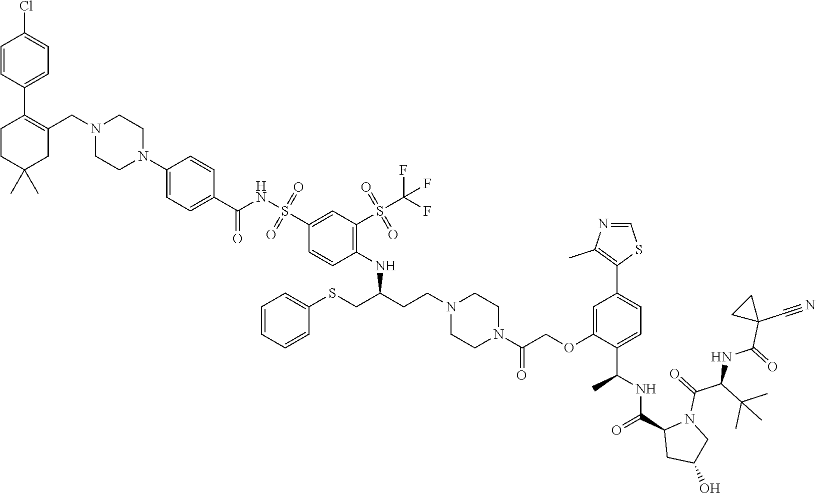 [SMILES: Cc1ncsc1-c1ccc([C@H](C)NC(=O)[C@@H]2C[C@@H](O)CN2C(=O)[C@@H](NC(=O)C2(C#N)CC2)C(C)(C)C)c(OCC(=O)N2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-221 | 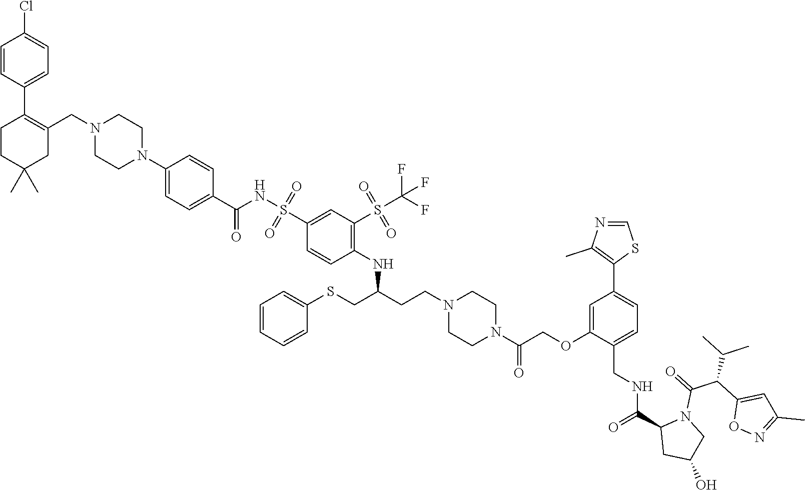 [SMILES: Cc1cc([C@H](C(=O)N2C[C@H](O)C[C@H]2C(=O)NCc2ccc(-c3scnc3C)cc2OCC(=O)N2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)C)on1] |
| I-222 | 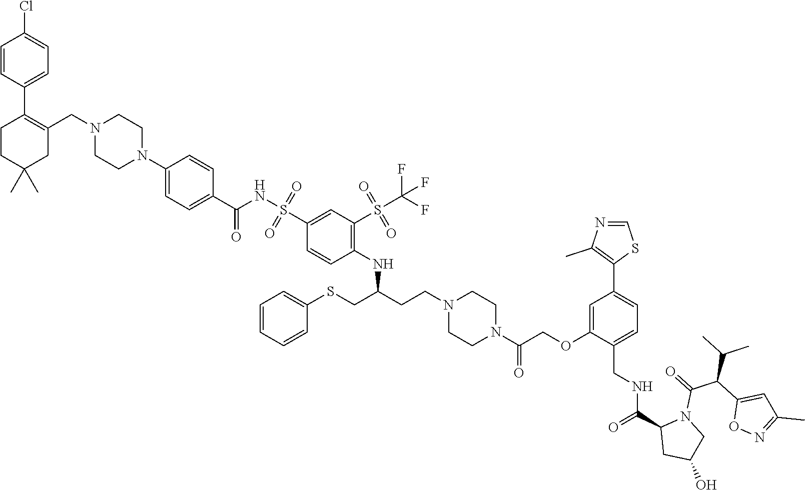 [SMILES: Cc1cc([C@@H](C(=O)N2C[C@H](O)C[C@H]2C(=O)NCc2ccc(-c3scnc3C)cc2OCC(=O)N2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)C)on1] |
| I-223 | 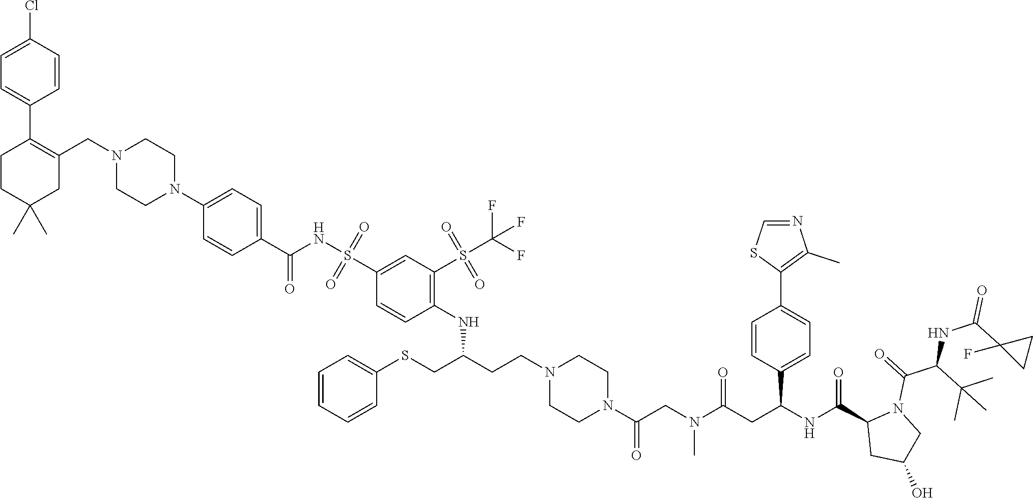 [SMILES: Cc1ncsc1-c1ccc([C@H](CC(=O)N(C)CC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@@H]2C[C@@H](O)CN2C(=O)[C@@H](NC(=O)C2(F)CC2)C(C)(C)C)cc1] |
| I-224 | 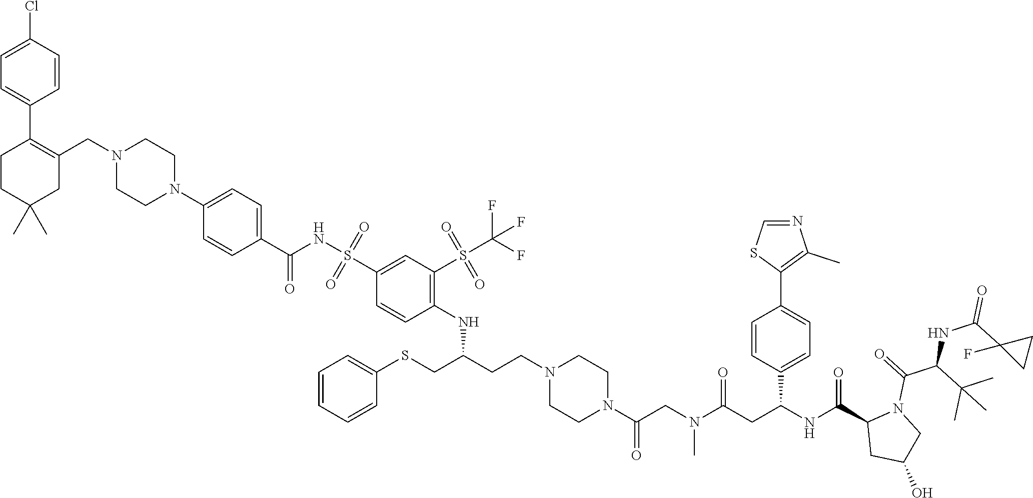 [SMILES: Cc1ncsc1-c1ccc([C@@H](CC(=O)N(C)CC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@@H]2C[C@@H](O)CN2C(=O)[C@@H](NC(=O)C2(F)CC2)C(C)(C)C)cc1] |
| I-225 | 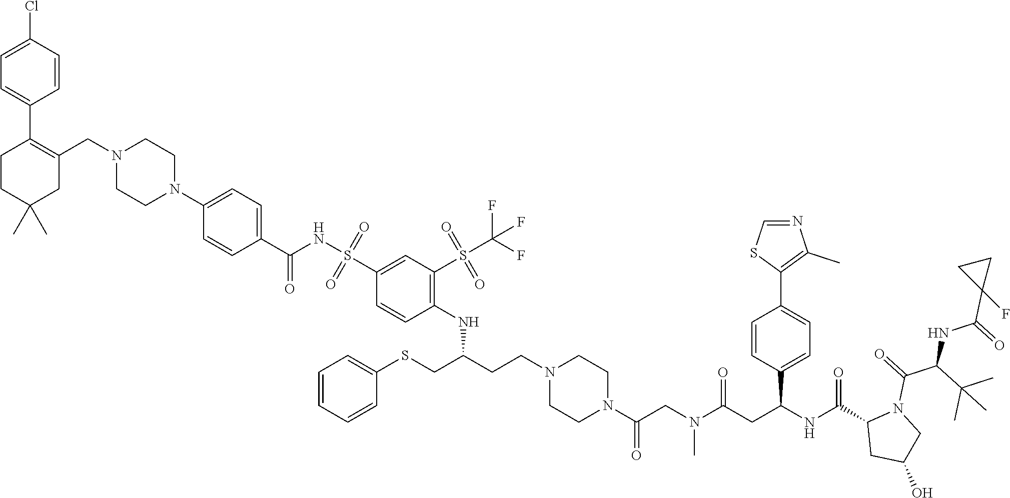 [SMILES: Cc1ncsc1-c1ccc([C@H](CC(=O)N(C)CC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@H]2C[C@@H](O)CN2C(=O)[C@@H](NC(=O)C2(F)CC2)C(C)(C)C)cc1] |
| I-226 | 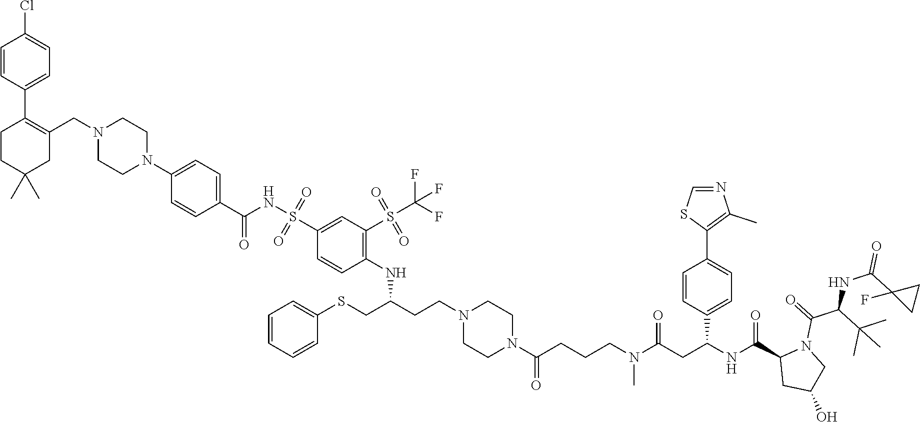 [SMILES: Cc1ncsc1-c1ccc([C@@H](CC(=O)N(C)CCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@@H]2C[C@@H](O)CN2C(=O)[C@@H](NC(=O)C2(F)CC2)C(C)(C)C)cc1] |
| I-227 | 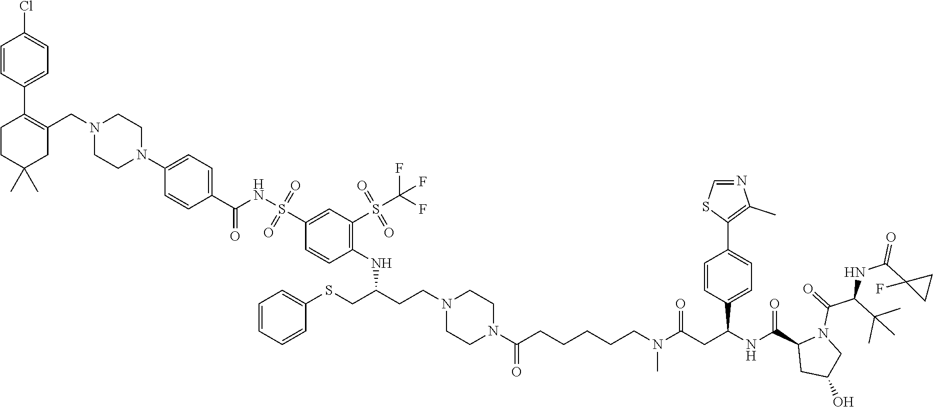 [SMILES: Cc1ncsc1-c1ccc([C@H](CC(=O)N(C)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@@H]2C[C@@H](O)CN2C(=O)[C@@H](NC(=O)C2(F)CC2)C(C)(C)C)cc1] |
| I-228 | 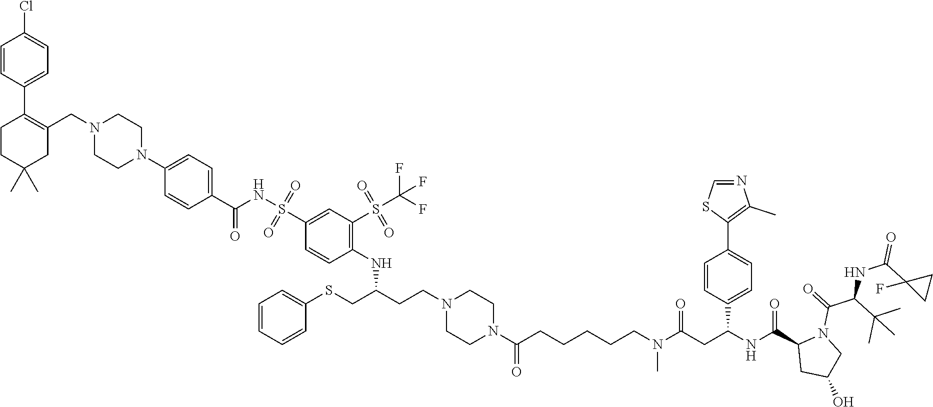 [SMILES: Cc1ncsc1-c1ccc([C@@H](CC(=O)N(C)CCCCCC(=O)N2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)NC(=O)[C@@H]2C[C@@H](O)CN2C(=O)[C@@H](NC(=O)C2(F)CC2)C(C)(C)C)cc1] |
| I-229 | 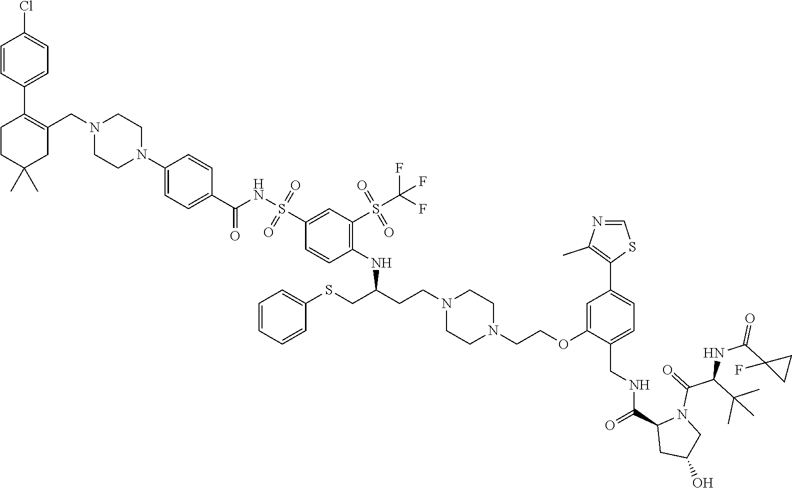 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-230 | 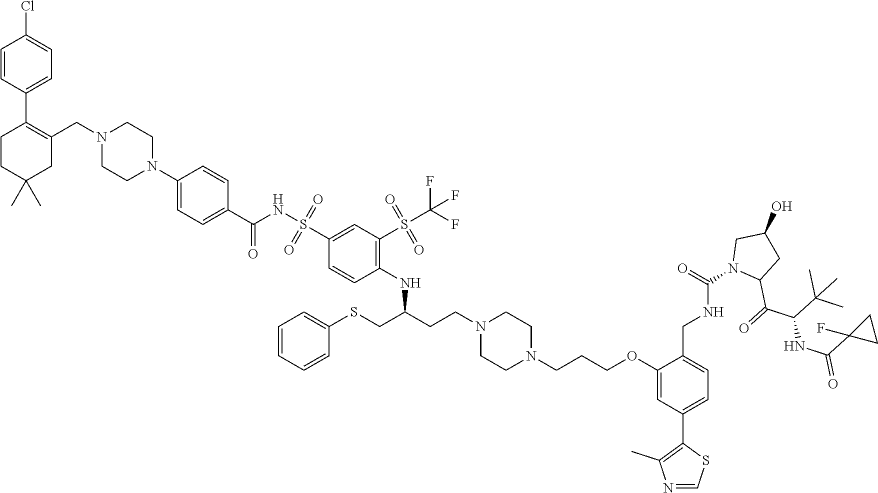 |
| I-231 | 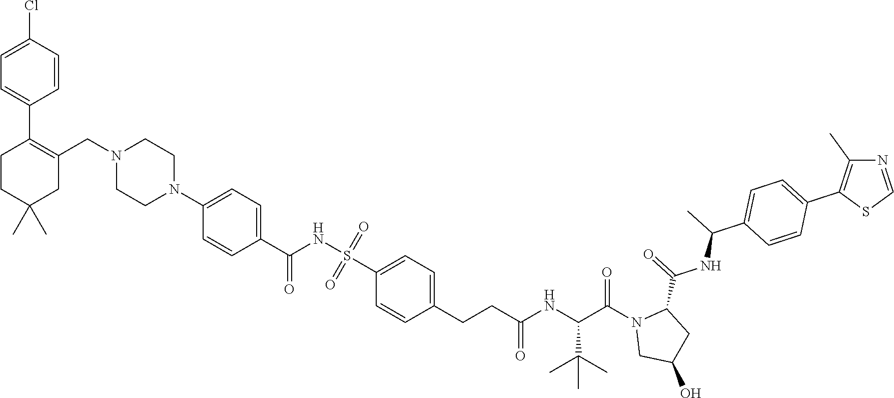 [SMILES: Cc1ncsc1-c1ccc([C@H](C)NC(=O)[C@@H]2C[C@@H](O)CN2C(=O)[C@@H](NC(=O)CCc2ccc(S(=O)(=O)NC(=O)c3ccc(N4CCN(CC5=C(c6ccc(Cl)cc6)CCC(C)(C)C5)CC4)cc3)cc2)C(C)(C)C)cc1] |
| I-232 | 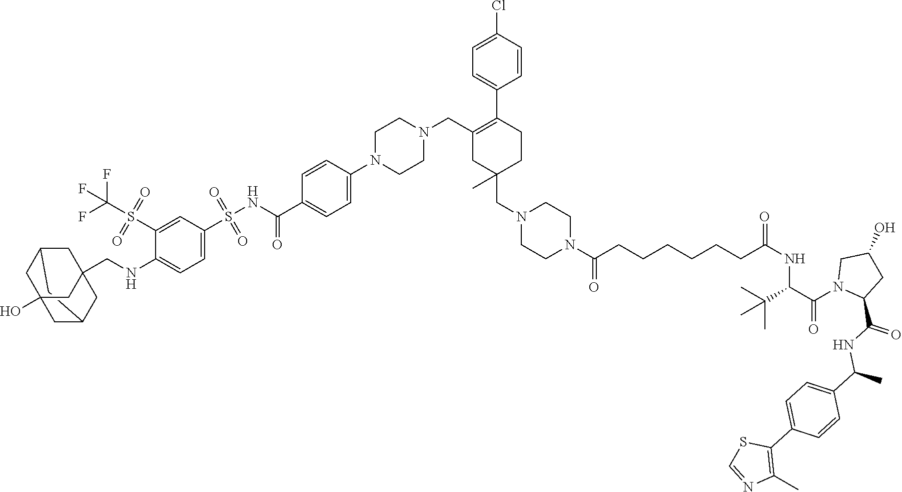 [SMILES: Cc1ncsc1-c1ccc([C@H](C)NC(=O)[C@@H]2C[C@@H](O)CN2C(=O)[C@@H](NC(=O)CCCCCCC(=O)N2CCN(CC3(C)CCC(c4ccc(Cl)cc4)=C(CN4CCN(c5ccc(C(=O)NS(=O)(=O)c6ccc(NCC78CC9CC(CC(O)(C9)C7)C8)c(S(=O)(=O)C(F)(F)F)c6)cc5)CC4)C3)CC2)C(C)(C)C)cc1] |
| I-233 | 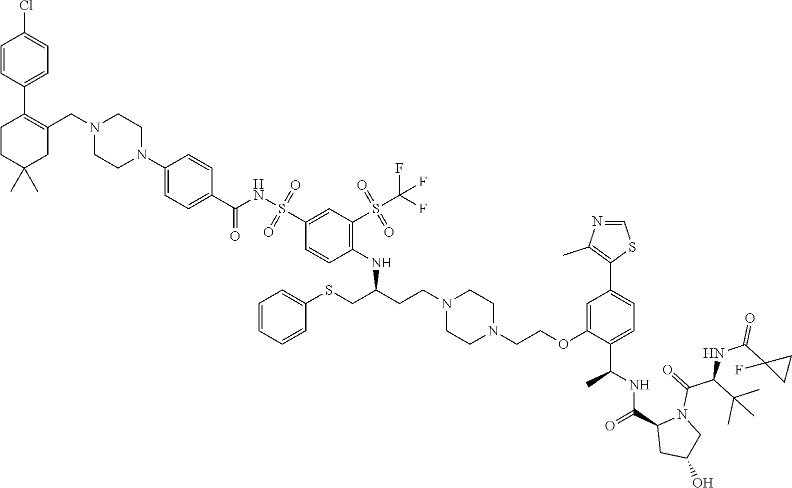 [SMILES: Cc1ncsc1-c1ccc([C@H](C)NC(=O)[C@@H]2C[C@@H](O)CN2C(=O)[C@@H](NC(=O)C2(F)CC2)C(C)(C)C)c(OCCN2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-234 | 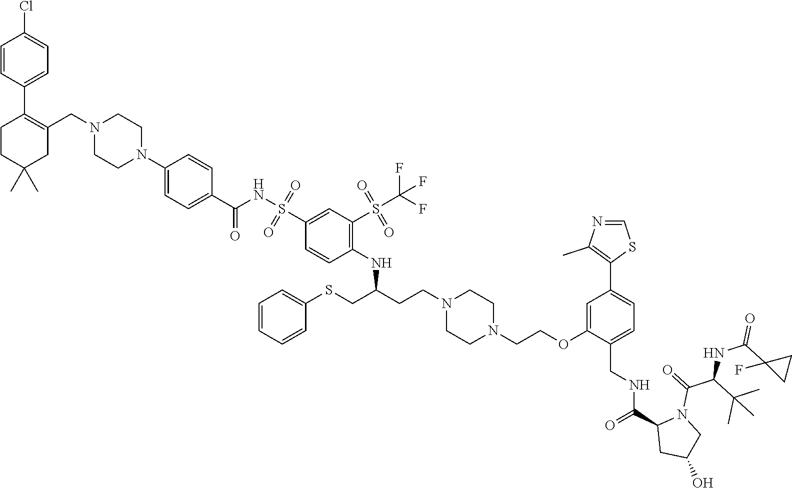 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-235 | 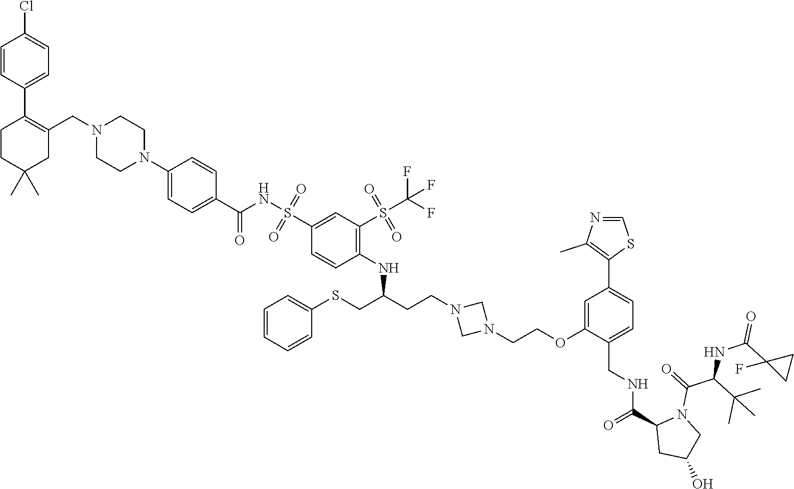 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)C2)c1] |
| I-236 | 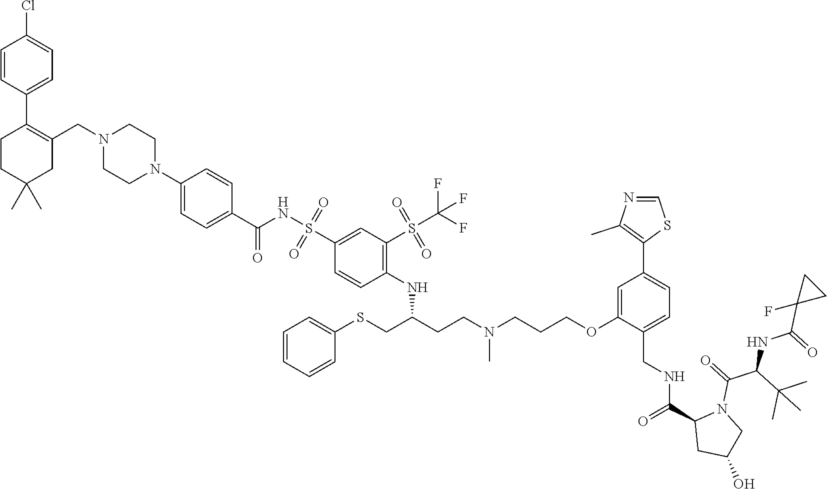 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCN(C)CC[C@H](CSc2ccccc2)Nc2ccc(S(=O)(=O)NC(=O)c3ccc(N4CCN(CC5=C(c6ccc(Cl)cc6)CCC(C)(C)C5)CC4)cc3)cc2S(=O)(=O)C(F)(F)F)c1] |
| I-237 | 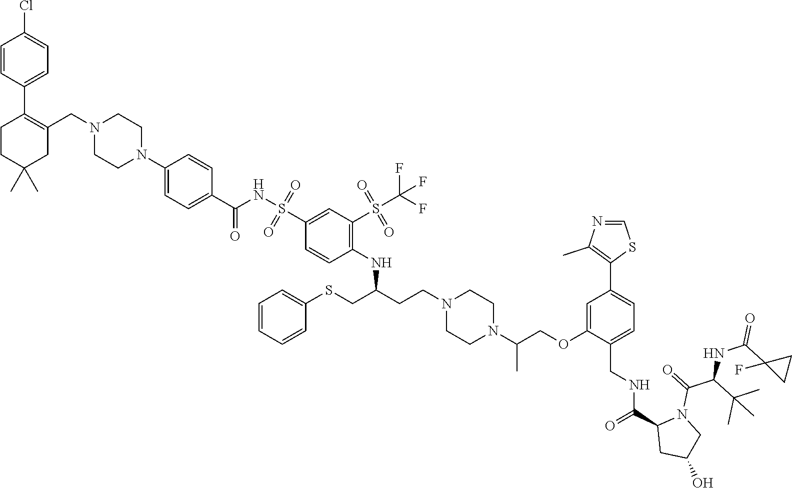 [SMILES: Cc1ncsc1-c1ccc(CNC(=O)[C@@H]2C[C@@H](O)CN2C(=O)[C@@H](NC(=O)C2(F)CC2)C(C)(C)C)c(OCC(C)N2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-238 | 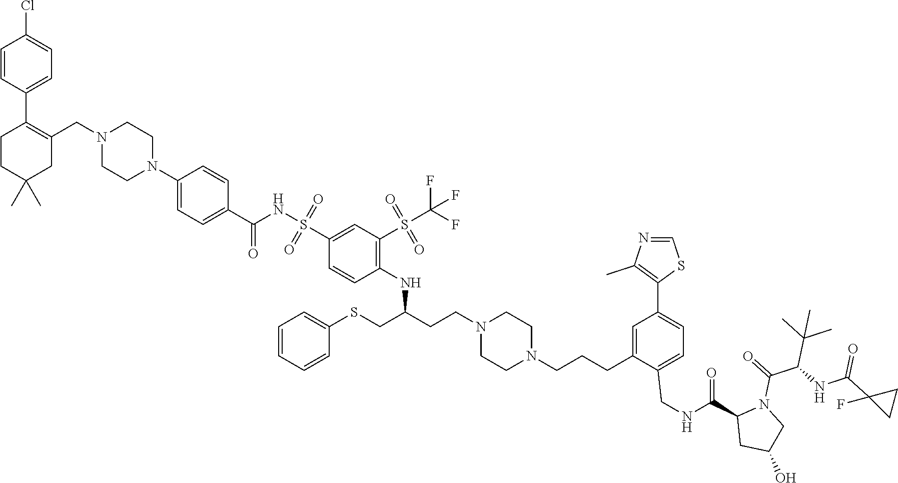 [SMILES: Cc1ncsc1-c1ccc(CNC(=O)[C@@H]2C[C@@H](O)CN2C(=O)[C@@H](NC(=O)C2(F)CC2)C(C)(C)C)c(CCCN2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-239 | 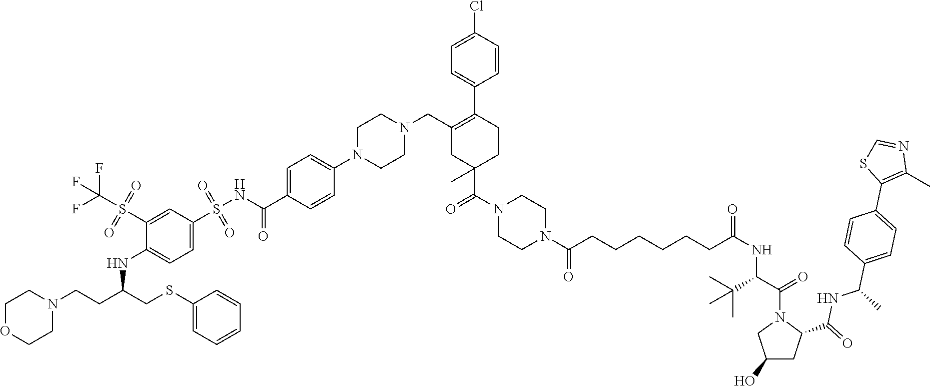 [SMILES: Cc1ncsc1-c1ccc([C@H](C)NC(=O)[C@@H]2C[C@@H](O)CN2C(=O)[C@@H](NC(=O)CCCCCCC(=O)N2CCN(C(=O)C3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] |
| I-240 | 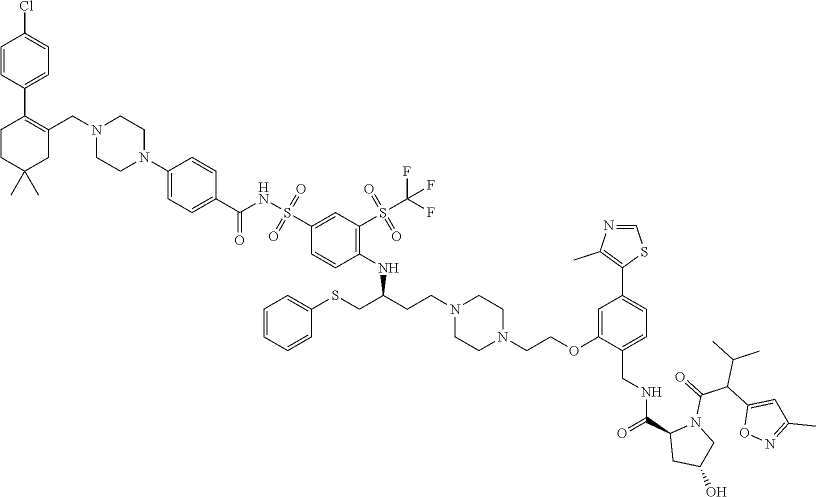 [SMILES: Cc1cc(C(C(=O)N2C[C@H](O)C[C@H]2C(=O)NCc2ccc(-c3scnc3C)cc2OCCN2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)C(C)C)on1] |
| I-241 | 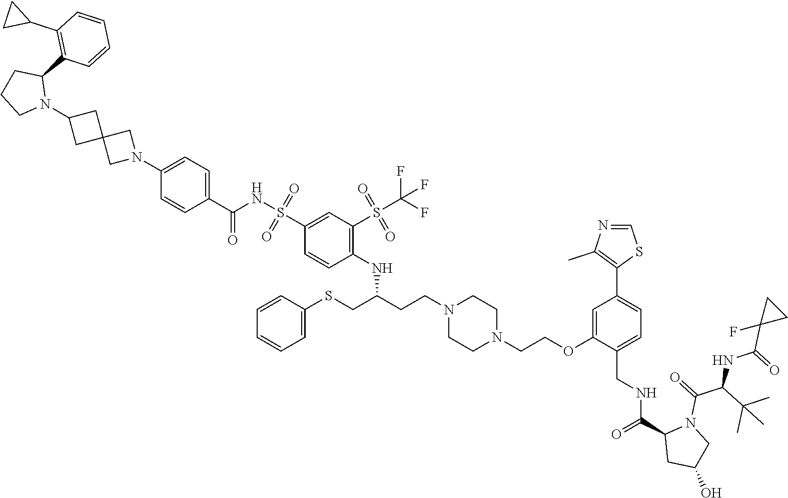 [SMILES: Cc1ncsc1-c1ccc(CNC(=O)[C@@H]2C[C@@H](O)CN2C(=O)[C@@H](NC(=O)C2(F)CC2)C(C)(C)C)c(OCCN2CCN(CC[C@H](CSc3ccccc3)Nc3ccc(S(=O)(=O)NC(=O)c4ccc(N5CC6(CC(N7CCC[C@H]7c7ccccc7C7CC7)C6)C5)cc4)cc3S(=O)(=O)C(F)(F)F)CC2)c1] |
| I-242 | 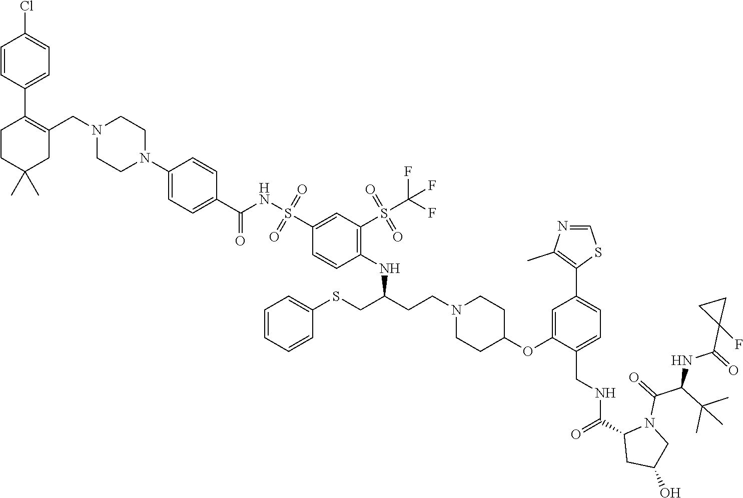 [SMILES: Cc1ncsc1-c1ccc(CNC(=O)[C@H]2C[C@@H](O)CN2C(=O)[C@@H](NC(=O)C2(F)CC2)C(C)(C)C)c(OC2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-243 | 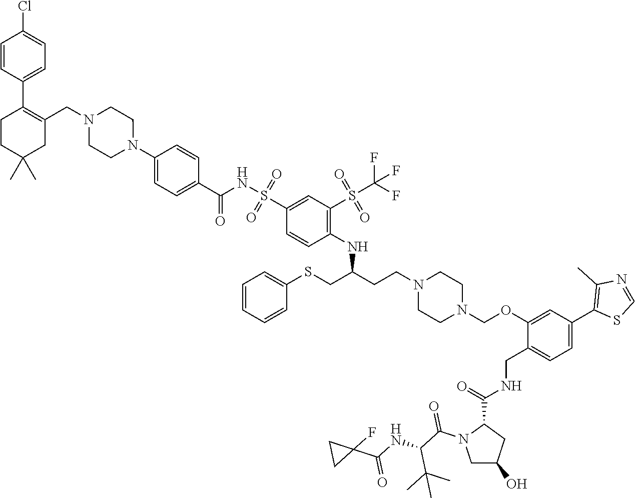 [SMILES: Cc1ncsc1-c1ccc(CNC(=O)[C@@H]2C[C@@H](O)CN2C(=O)[C@@H](NC(=O)C2(F)CC2)C(C)(C)C)c(OCN2CCN(CC[C@@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1] |
| I-245 | 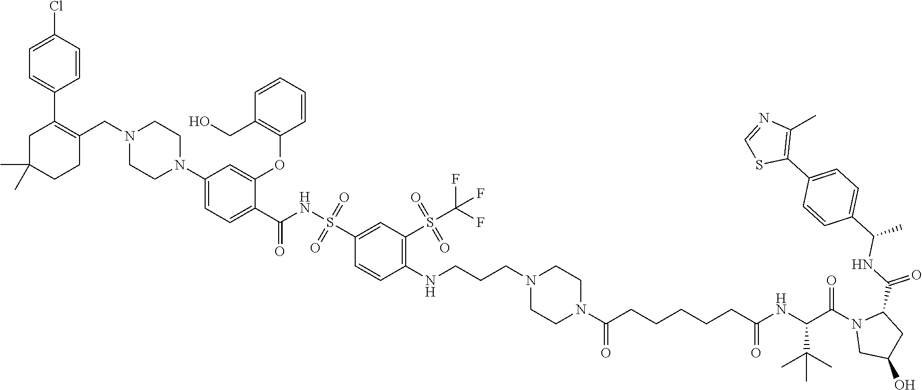 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4ccccc4CO)cc3S(=O)(=O)C(F)(F)F)CC2)C(C)(C)C)cc1] |
| I-246 | 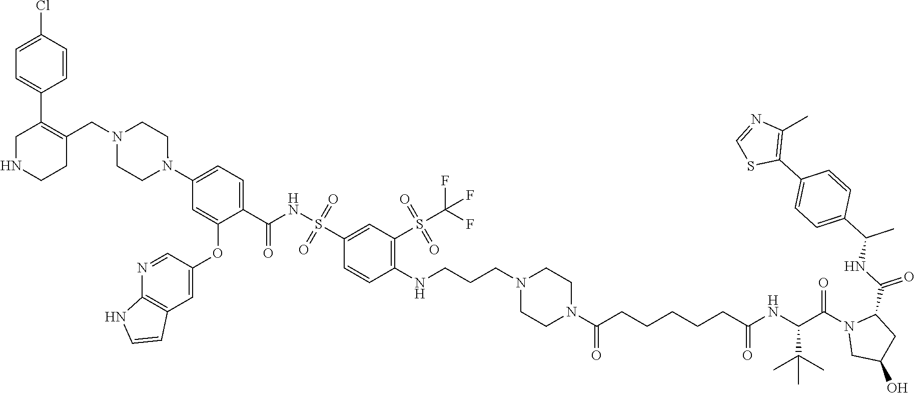 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CNCC6)CC5)cc4Oc4cnc5[nH]ccc5c4)cc3S(=O)(=O)C(F)(F)F)CC2)C(C)(C)C)cc1] |
| I-247 | 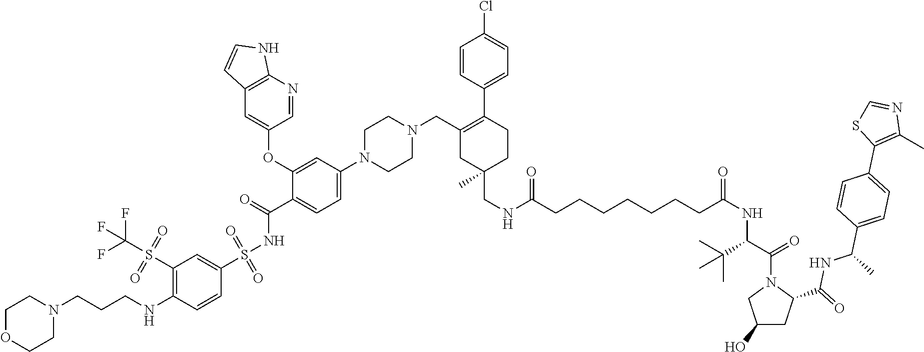 [SMILES: Cc1ncsc1-c1ccc([C@H](C)NC(=O)[C@@H]2C[C@@H](O)CN2C(=O)[C@@H](NC(=O)CCCCCCCC(=O)NC[C@]2(C)CCC(c3ccc(Cl)cc3)=C(CN3CCN(c4ccc(C(=O)NS(=O)(=O)c5ccc(NCCCN6CCOCC6)c(S(=O)(=O)C(F)(F)F)c5)c(Oc5cnc6[nH]ccc6c5)c4)CC3)C2)C(C)(C)C)cc1] |
| I-248 | 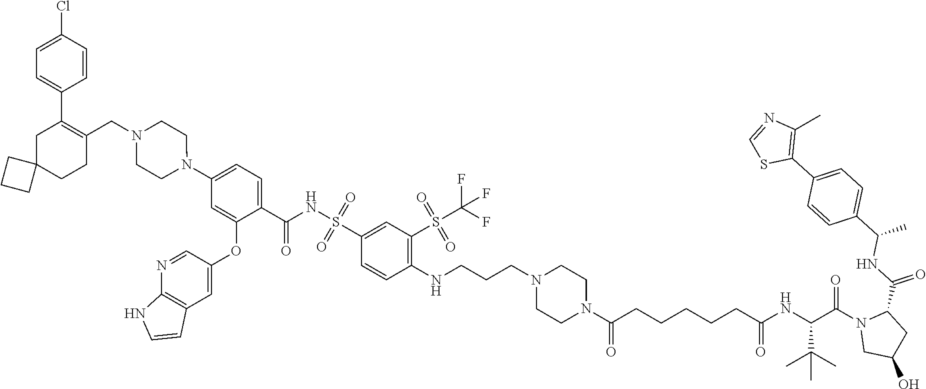 [SMILES: Cc1ncsc1-c1ccc([C@H](C)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CC7(CCC7)CC6)CC5)cc4Oc4cnc5[nH]ccc5c4)cc3S(=O)(=O)C(F)(F)F)CC2)C(C)(C)C)cc1] |
| I-249 | 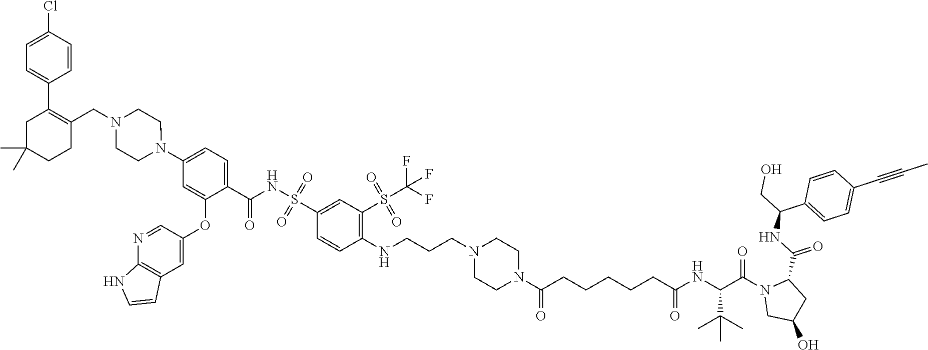 [SMILES: CC#Cc1ccc([C@H](CO)NC(=O)[C@@H]2C[C@@H](O)CN2C(=O)[C@@H](NC(=O)CCCCCC(=O)N2CCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-250 | 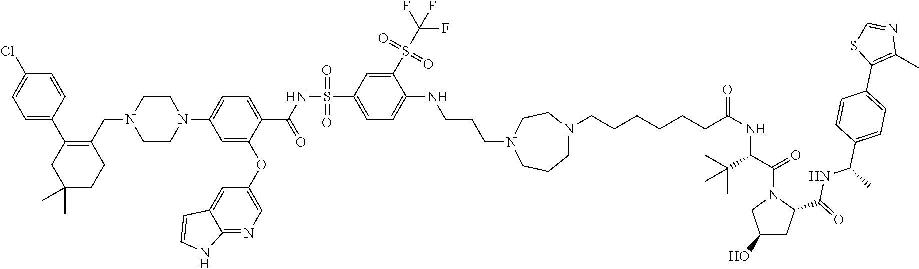 [SMILES: Cc1ncsc1-c1ccc([C@H](C)NC(=O)[C@@H]2C[C@@H](O)CN2C(=O)[C@@H](NC(=O)CCCCCCN2CCCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-251 | 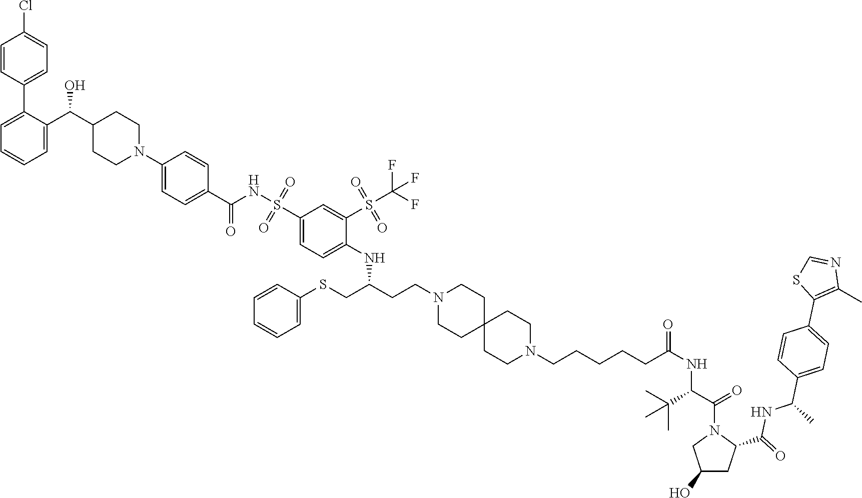 [SMILES: Cc1ncsc1-c1ccc([C@H](C)NC(=O)[C@@H]2C[C@@H](O)CN2C(=O)[C@@H](NC(=O)CCCCCN2CCC3(CC2)CCN(CC[C@H](CSc2ccccc2)Nc2ccc(S(=O)(=O)NC(=O)c4ccc(N5CCC([C@@H](O)c6ccccc6-c6ccc(Cl)cc6)CC5)cc4)cc2S(=O)(=O)C(F)(F)F)CC3)C(C)(C)C)cc1] |
| I-252 | 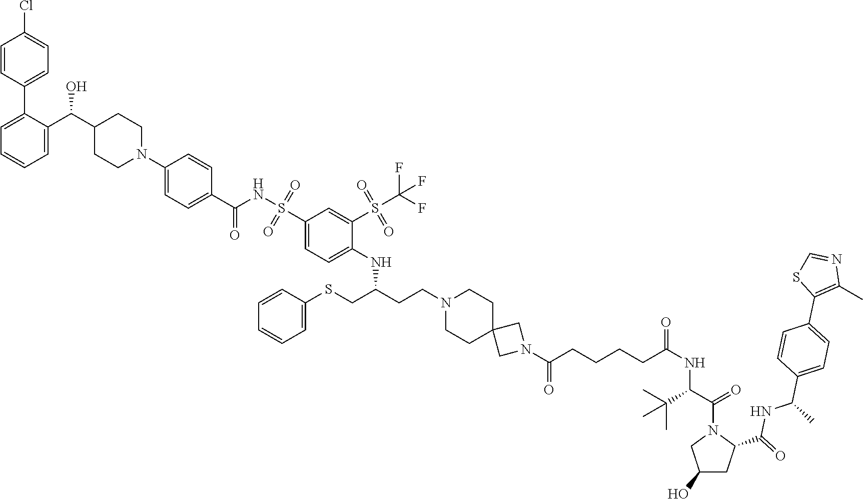 [SMILES: Cc1ncsc1-c1ccc([C@H](C)NC(=O)[C@@H]2C[C@@H](O)CN2C(=O)[C@@H](NC(=O)CCCCC(=O)N2CC3(CCN(CC[C@H](CSc4ccccc4)Nc4ccc(S(=O)(=O)NC(=O)c5ccc(N6CCC([C@@H](O)c7ccccc7-c7ccc(Cl)cc7)CC6)cc5)cc4S(=O)(=O)C(F)(F)F)CC3)C2)C(C)(C)C)cc1] |
| I-253 | 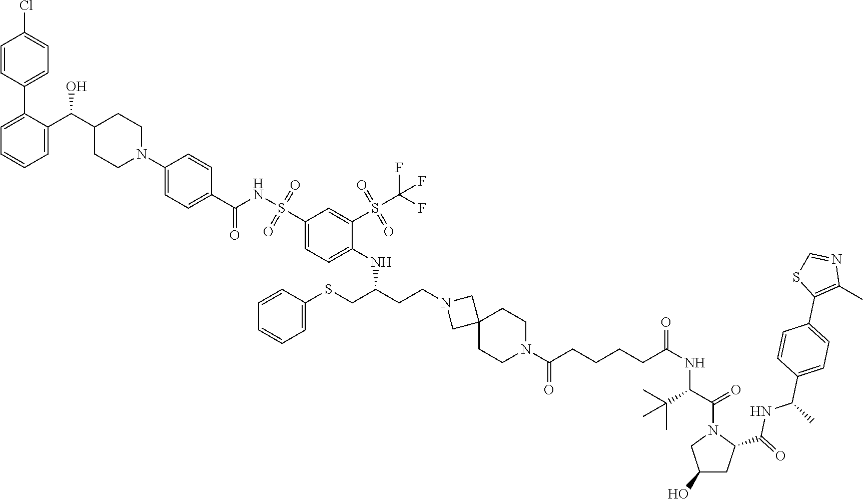 [SMILES: Cc1ncsc1-c1ccc([C@H](C)NC(=O)[C@@H]2C[C@@H](O)CN2C(=O)[C@@H](NC(=O)CCCCC(=O)N2CCC3(CC2)CN(CC[C@H](CSc2ccccc2)Nc2ccc(S(=O)(=O)NC(=O)c4ccc(N5CCC([C@@H](O)c6ccccc6-c6ccc(Cl)cc6)CC5)cc4)cc2S(=O)(=O)C(F)(F)F)C3)C(C)(C)C)cc1] |
| I-254 | 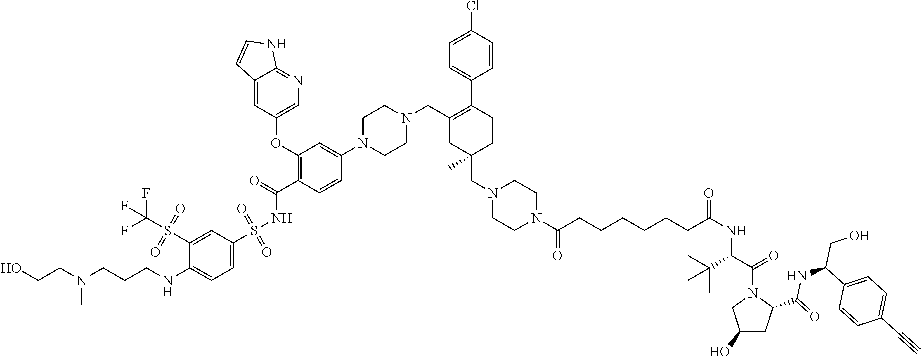 [SMILES: C#Cc1ccc([C@H](CO)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NCCCN(C)CCO)c(S(=O)(=O)C(F)(F)F)c6)c(Oc6cnc7[nH]ccc7c6)c5)CC4)C3)CC2)C(C)(C)C)cc1] |
| I-255 | 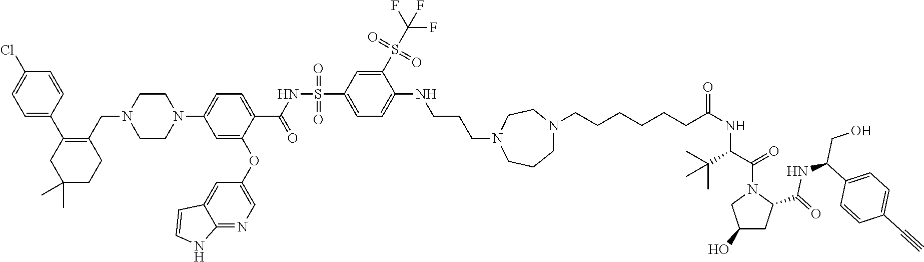 [SMILES: C#Cc1ccc([C@H](CO)NC(=O)[C@@H]2C[C@@H](O)CN2C(=O)[C@@H](NC(=O)CCCCCCN2CCCN(CCCNc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-256 | 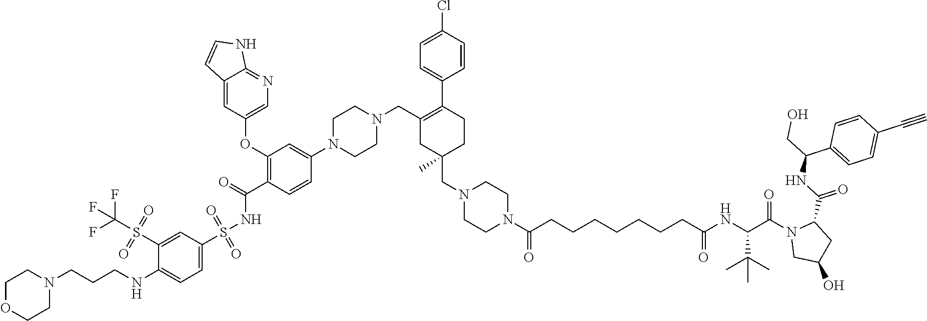 [SMILES: C#Cc1ccc([C@H](CO)NC(=O)[C@@H]2C[C@@H](O)CN2C(=O)[C@@H](NC(=O)CCCCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-257 | 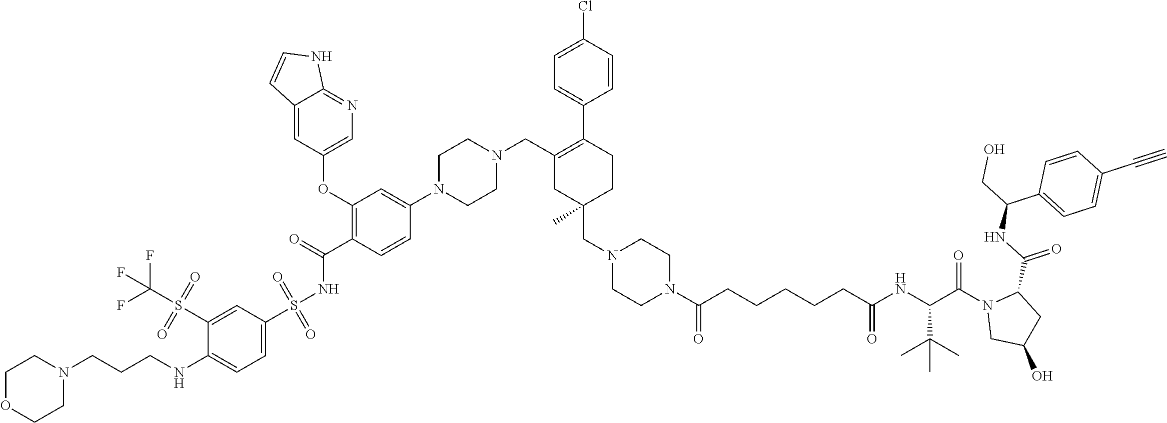 [SMILES: C#Cc1ccc([C@H](CO)NC(=O)[C@@H]2C[C@@H](O)CN2C(=O)[C@@H](NC(=O)CCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-258 | 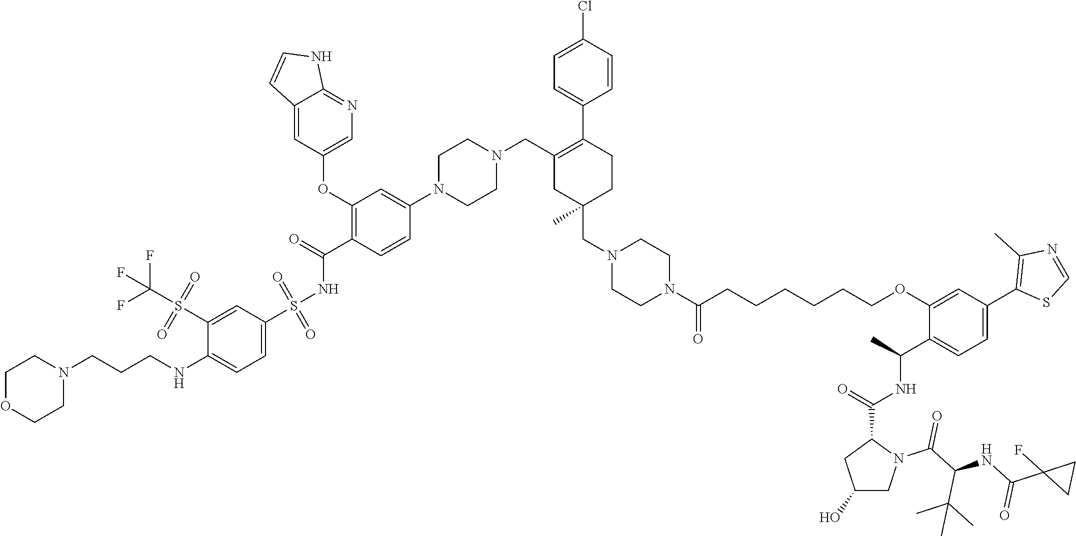 [SMILES: Cc1ncsc1-c1ccc([C@H](C)NC(=O)[C@H]2C[C@@H](O)CN2C(=O)[C@@H](NC(=O)C2(F)CC2)C(C)(C)C)c(OCCCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)c1] |
| I-259 | 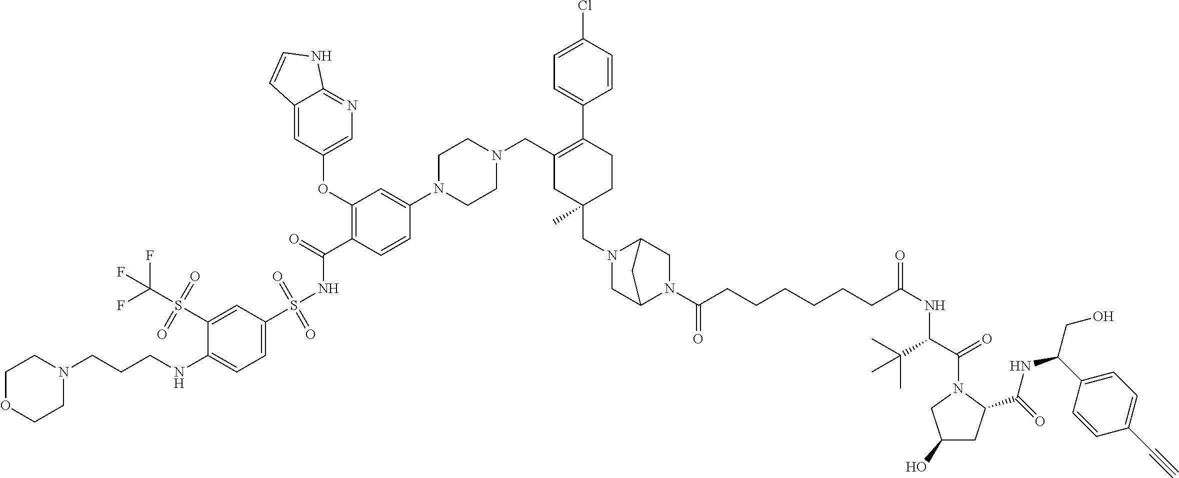 [SMILES: C#Cc1ccc([C@H](CO)NC(=O)[C@@H]2C[C@@H](O)CN2C(=O)[C@@H](NC(=O)CCCCCCC(=O)N2CC3CC2CN3C[C@]2(C)CCC(c3ccc(Cl)cc3)=C(CN3CCN(c4ccc(C(=O)NS(=O)(=O)c5ccc(NCCCN6CCOCC6)c(S(=O)(=O)C(F)(F)F)c5)c(Oc5cnc6[nH]ccc6c5)c4)CC3)C2)C(C)(C)C)cc1] |
| I-260 | 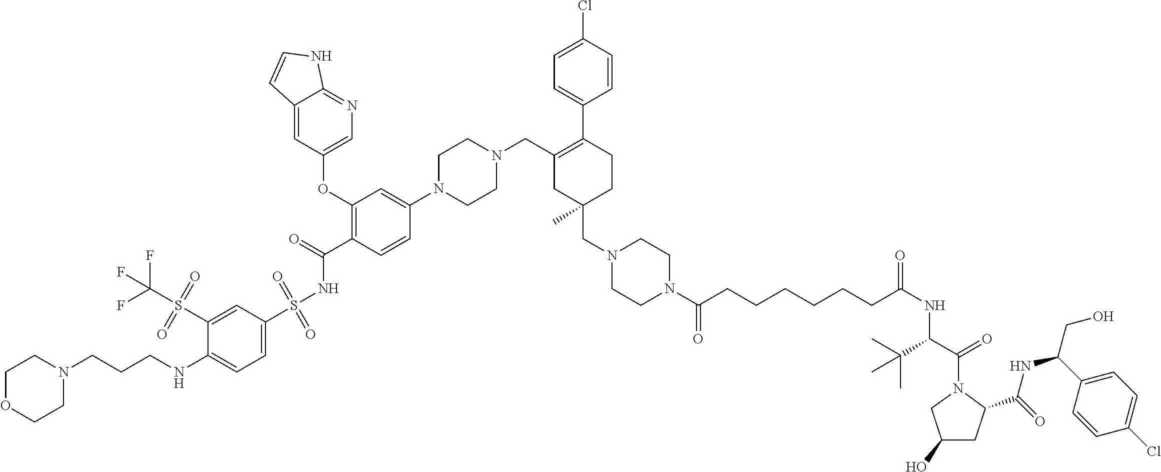 [SMILES: CC(C)(C)[C@H](NC(=O)CCCCCCC(=O)N1CCN(C[C@]2(C)CCC(c3ccc(Cl)cc3)=C(CN3CCN(c4ccc(C(=O)NS(=O)(=O)c5ccc(NCCCN6CCOCC6)c(S(=O)(=O)C(F)(F)F)c5)c(Oc5cnc6[nH]ccc6c5)c4)CC3)C2)CC1)C(=O)N1C[C@H](O)C[C@H]1C(=O)N[C@@H](CO)c1ccc(Cl)cc1] |
| I-261 | 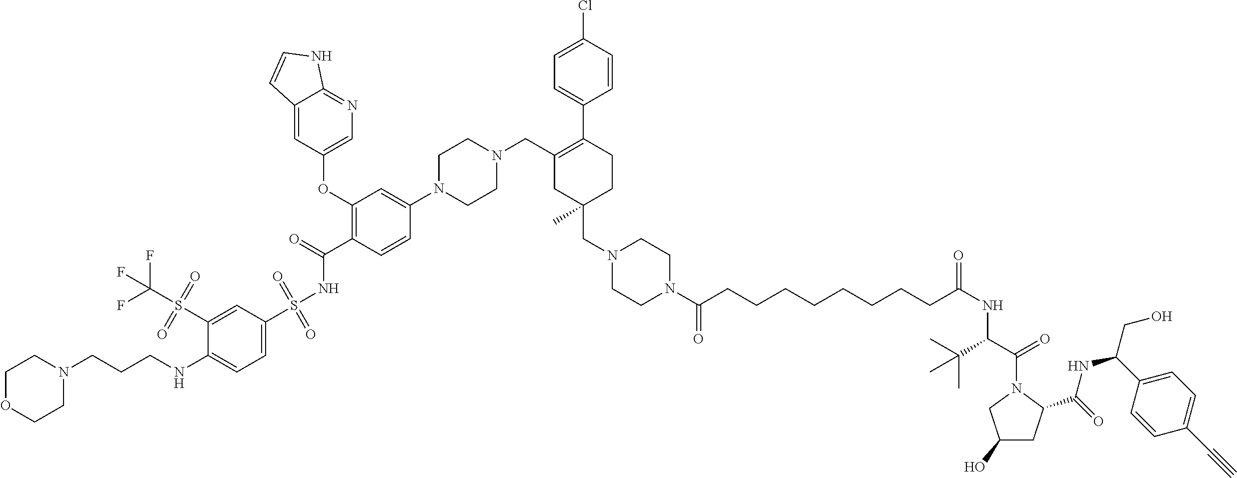 [SMILES: C#Cc1ccc([C@H](CO)NC(=O)[C@@H]2C[C@@H](O)CN2C(=O)[C@@H](NC(=O)CCCCCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-262 | 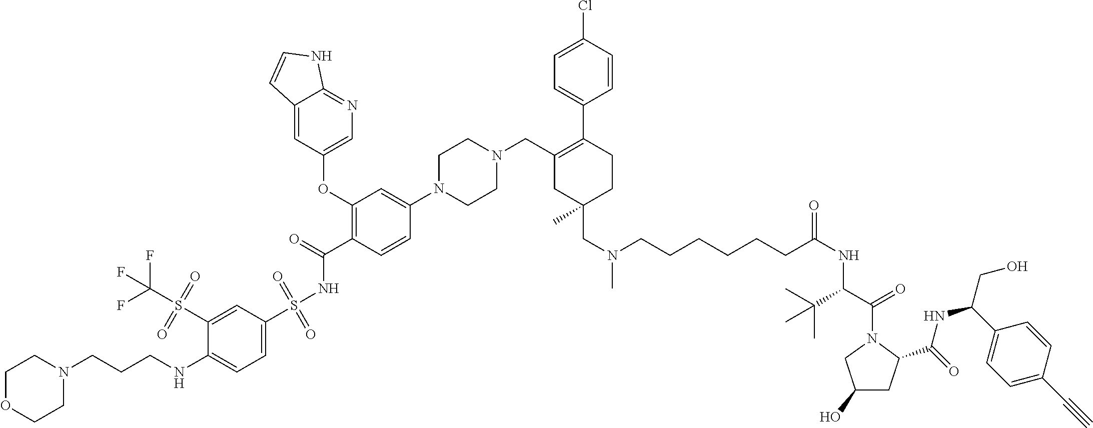 [SMILES: C#Cc1ccc([C@H](CO)NC(=O)[C@@H]2C[C@@H](O)CN2C(=O)[C@@H](NC(=O)CCCCCCN(C)C[C@]2(C)CCC(c3ccc(Cl)cc3)=C(CN3CCN(c4ccc(C(=O)NS(=O)(=O)c5ccc(NCCCN6CCOCC6)c(S(=O)(=O)C(F)(F)F)c5)c(Oc5cnc6[nH]ccc6c5)c4)CC3)C2)C(C)(C)C)cc1] |
| I-263 | 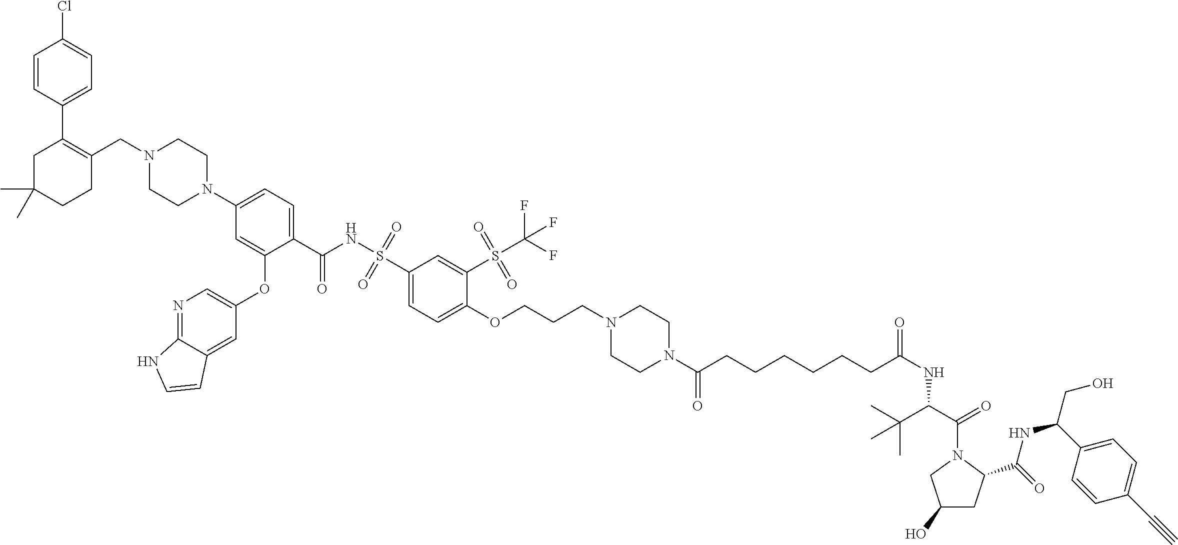 [SMILES: C#Cc1ccc([C@H](CO)NC(=O)[C@@H]2C[C@@H](O)CN2C(=O)[C@@H](NC(=O)CCCCCCC(=O)N2CCN(CCCOc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-264 | 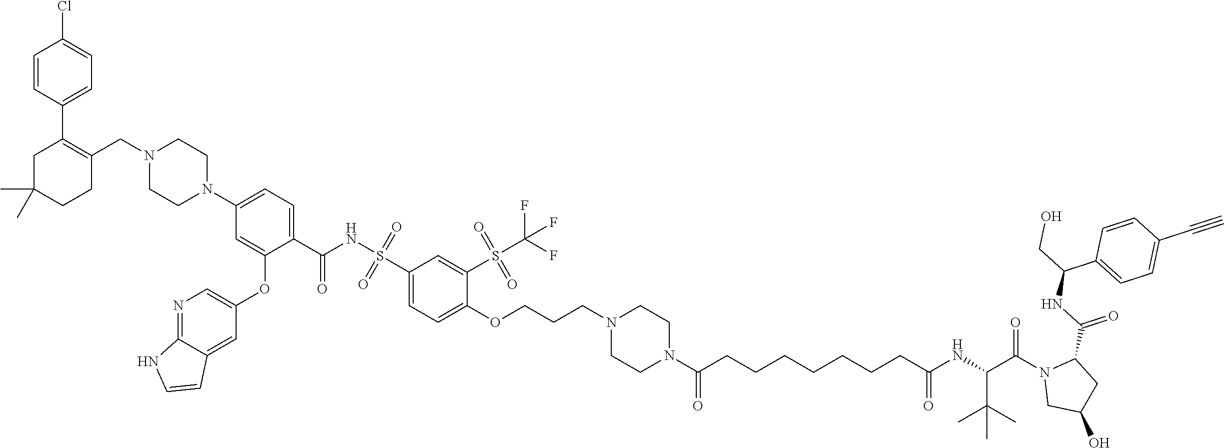 [SMILES: C#Cc1ccc([C@H](CO)NC(=O)[C@@H]2C[C@@H](O)CN2C(=O)[C@@H](NC(=O)CCCCCCCC(=O)N2CCN(CCCOc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-265 | 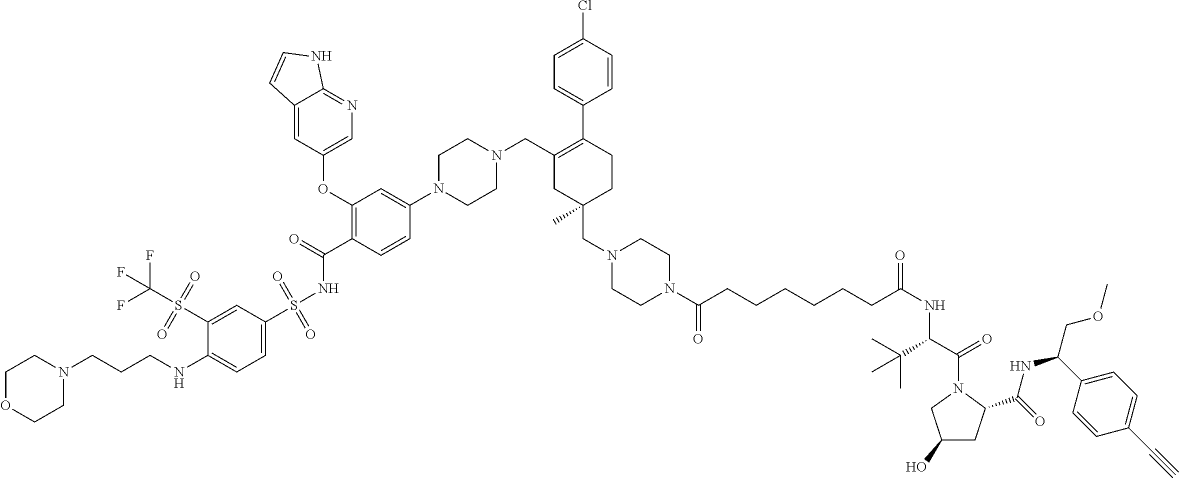 [SMILES: C#Cc1ccc([C@H](COC)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)cc1] |
| I-266 | 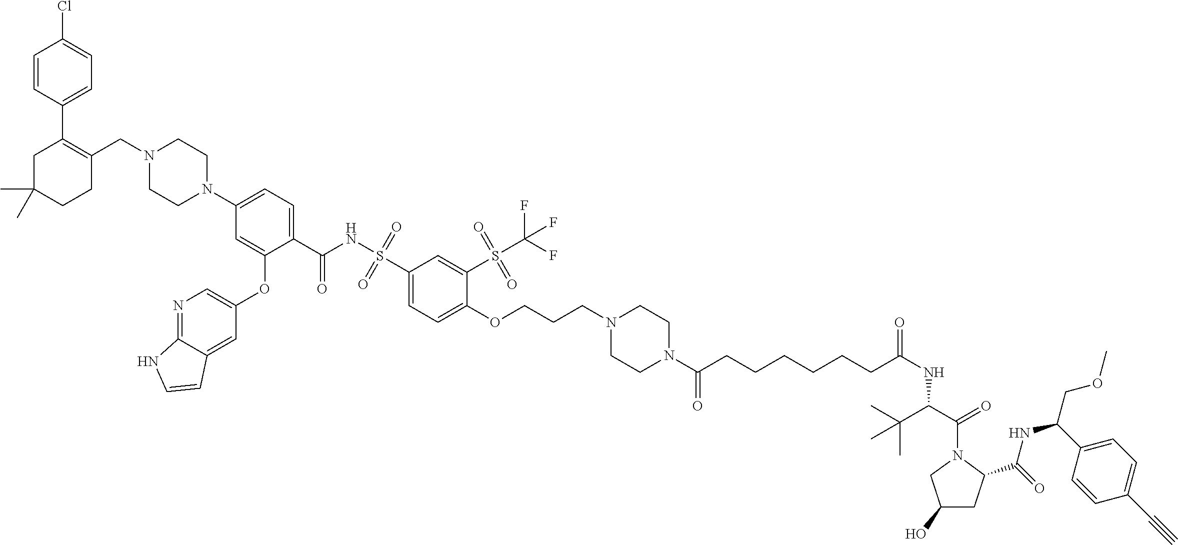 [SMILES: C#Cc1ccc([C@H](COC)NC(=O)[C@@H]2C[C@@H](O)CN2C(=O)[C@@H](NC(=O)CCCCCCC(=O)N2CCN(CCCOc3ccc(S(=O)(=O)NC(=O)c4ccc(N5CCN(CC6=C(c7ccc(Cl)cc7)CC(C)(C)CC6)CC5)cc4Oc4cnc5[nH]ccc5c4)cc3S(=O)(=O)C(F)(F)F)CC2)C(C)(C)C)cc1] |
| I-267 | 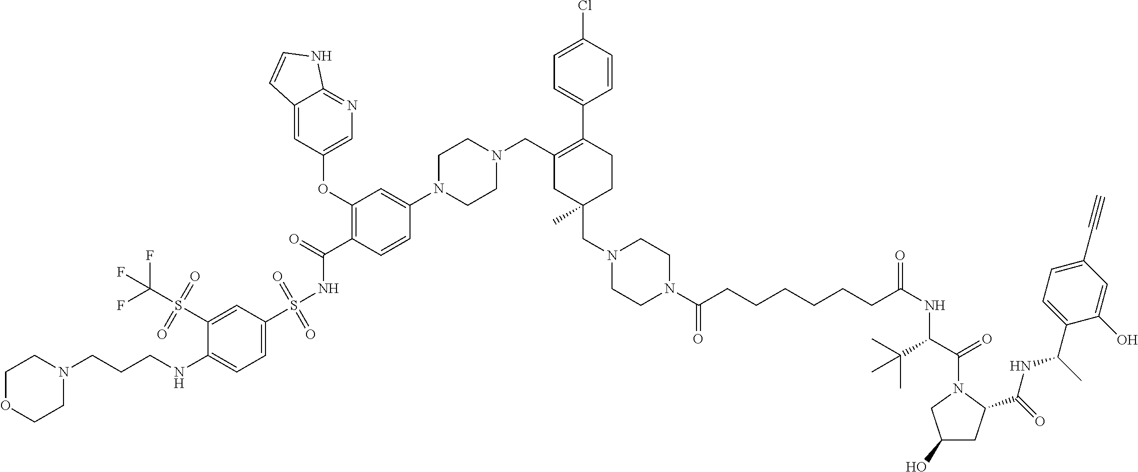 [SMILES: C#Cc1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)C(C)(C)C)c(O)c1] |
| I-268 | 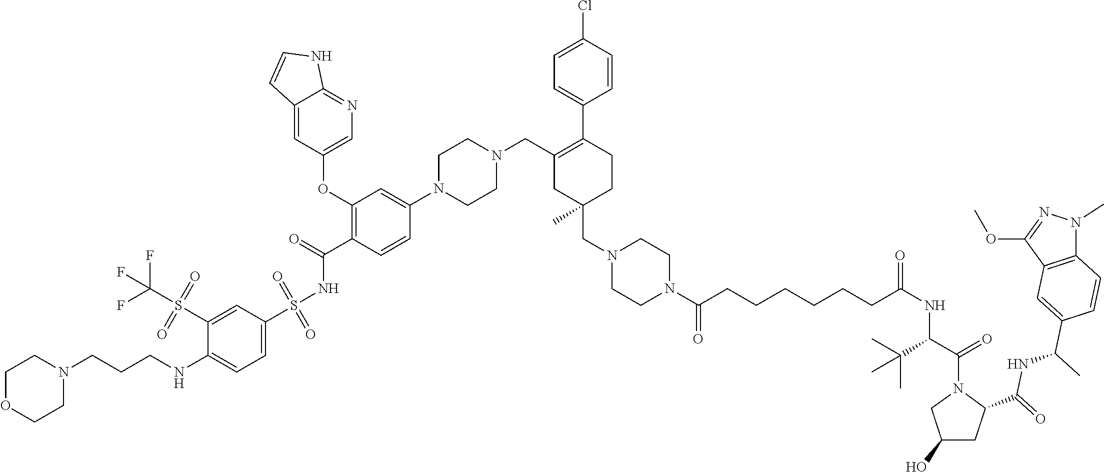 [SMILES: COc1nn(C)c2ccc([C@H](C)NC(=O)[C@@H]3C[C@@H](O)CN3C(=O)[C@@H](NC(=O)CCCCCCC(=O)N3CCN(C[C@]4(C)CCC(c5ccc(Cl)cc5)=C(CN5CCN(c6ccc(C(=O)NS(=O)(=O)c7ccc(NCCCN8CCOCC8)c(S(=O)(=O)C(F)(F)F)c7)c(Oc7cnc8[nH]ccc8c7)c6)CC5)C4)CC3)C(C)(C)C)cc12] |
| I-269 | 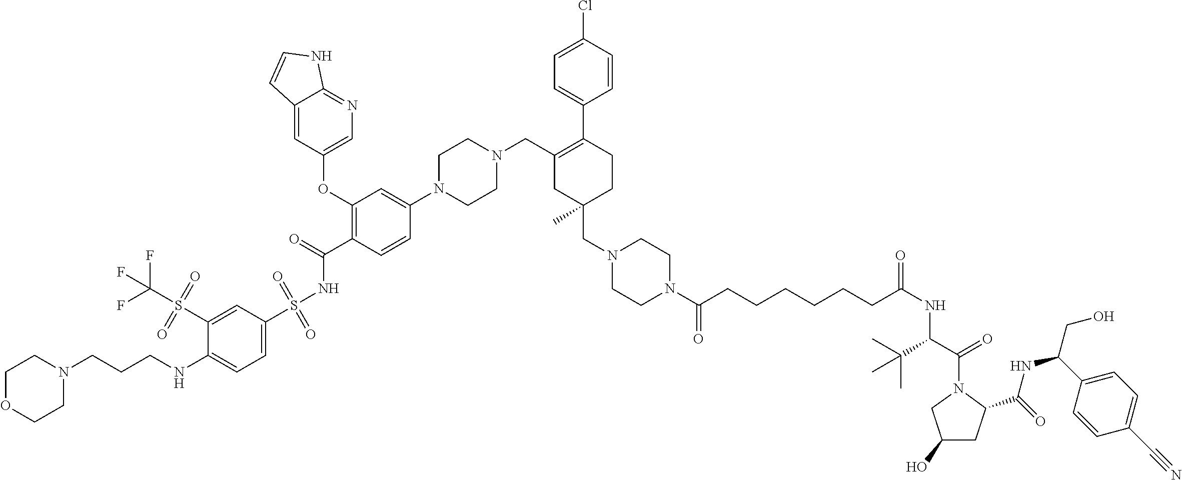 [SMILES: CC(C)(C)[C@H](NC(=O)CCCCCCC(=O)N1CCN(C[C@]2(C)CCC(c3ccc(Cl)cc3)=C(CN3CCN(c4ccc(C(=O)NS(=O)(=O)c5ccc(NCCCN6CCOCC6)c(S(=O)(=O)C(F)(F)F)c5)c(Oc5cnc6[nH]ccc6c5)c4)CC3)C2)CC1)C(=O)N1C[C@H](O)C[C@H]1C(=O)N[C@@H](CO)c1ccc(C#N)cc1] |
| I-270 | 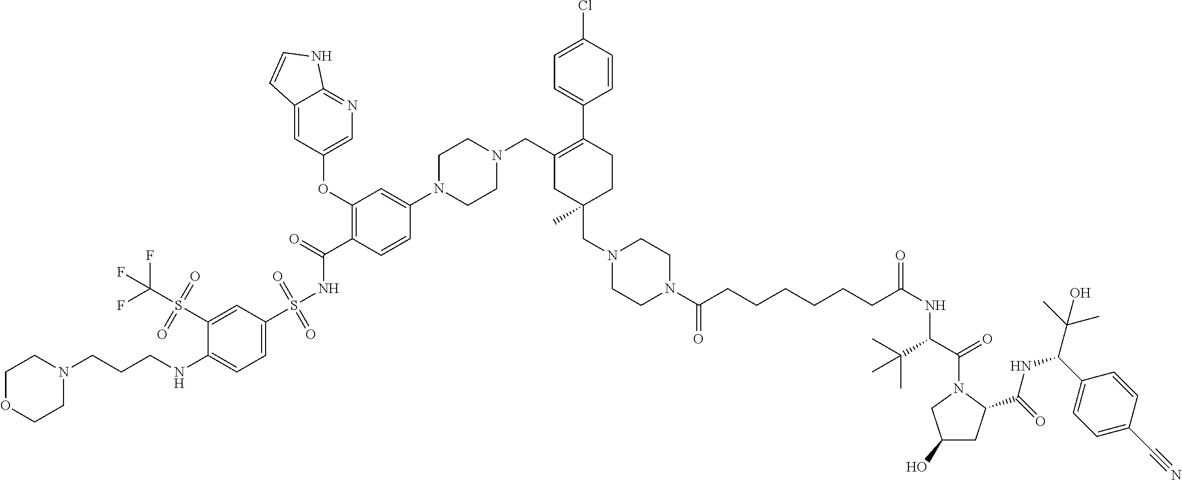 [SMILES: CC(C)(C)[C@H](NC(=O)CCCCCCC(=O)N1CCN(C[C@]2(C)CCC(c3ccc(Cl)cc3)=C(CN3CCN(c4ccc(C(=O)NS(=O)(=O)c5ccc(NCCCN6CCOCC6)c(S(=O)(=O)C(F)(F)F)c5)c(Oc5cnc6[nH]ccc6c5)c4)CC3)C2)CC1)C(=O)N1C[C@H](O)C[C@H]1C(=O)N[C@@H](c1ccc(C#N)cc1)C(C)(C)O] |
| I-271 | 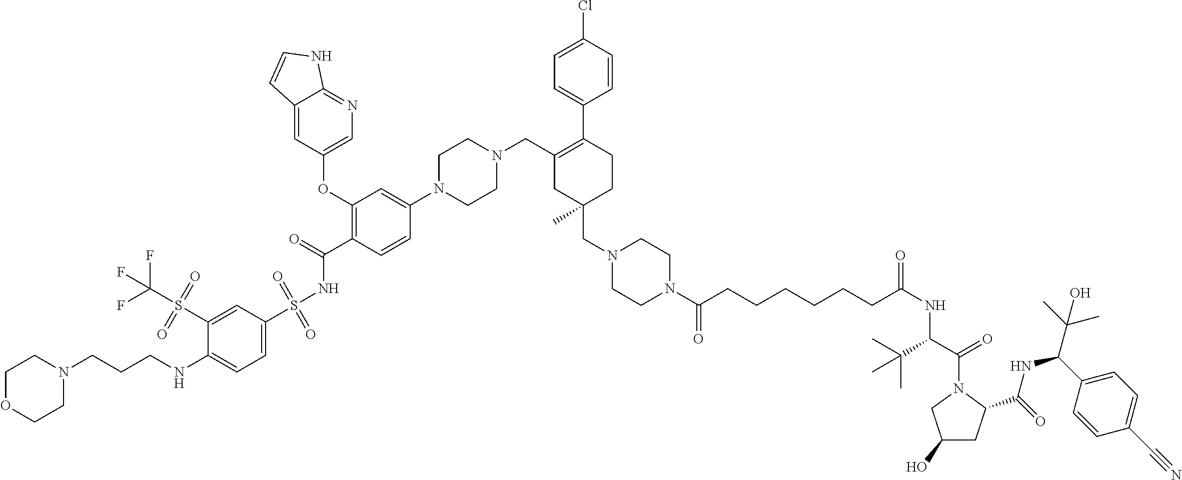 [SMILES: CC(C)(C)[C@H](NC(=O)CCCCCCC(=O)N1CCN(C[C@]2(C)CCC(c3ccc(Cl)cc3)=C(CN3CCN(c4ccc(C(=O)NS(=O)(=O)c5ccc(NCCCN6CCOCC6)c(S(=O)(=O)C(F)(F)F)c5)c(Oc5cnc6[nH]ccc6c5)c4)CC3)C2)CC1)C(=O)N1C[C@H](O)C[C@H]1C(=O)N[C@H](c1ccc(C#N)cc1)C(C)(C)O] |

In some embodiments, the present invention provides a compound set forth in Table 1, above, or a pharmaceutically acceptable salt thereof.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions. Mechanisms, and Structure, M. B. Smith and J. March 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio) pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis. T. W. Greene and P. G. M. Wuts, 3^{rd} edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In the schemes below, where a provided compound is formed having a reactive moiety (e.g., amine, alcohol, etc.), it is not shown but it is generally appreciated and well known by those having ordinary skill in the art that the reactivity of said reactive moiety may be masked by employing a suitable protecting group that can thereafter be removed in situ or during a separate synthetic step.

In certain embodiments, compounds of the present invention are generally prepared according to Scheme 1 set forth below:

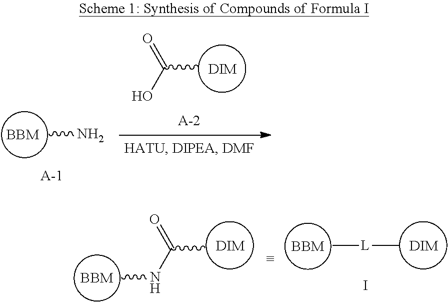

As depicted in Scheme 1, above, amine A-1 is coupled to acid A-2 using the coupling agent HATU in the presence of the base DIPEA in DMF to form a compound of formula I with a linker comprising an amide bond. The squiggly bond, [symbol] , represents the portion of the linker between BBM and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 2 set forth below:

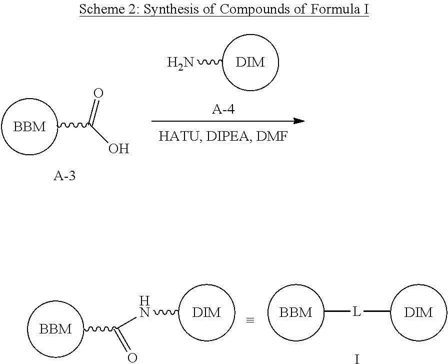

As depicted in Scheme 2, above, acid A-3 is coupled to amine A-4 using the coupling agent HATU in the presence of the base DIPEA in DMF to form a compound of formula I with a linker comprising an amide bond. The squiggly bond, [symbol] , represents the portion of the linker between BBM and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 3 set forth below:

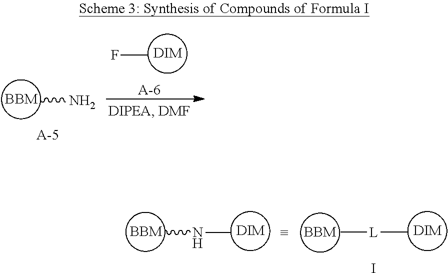

As depicted in Scheme 3, above, an S_{N}Ar displacement of fluoride A-6 by amine A-5 is effected in the presence of the base DIPEA in DMF to form a compound of formula I with a linker comprising a secondary amine. The squiggly bond, [symbol] , represents the portion of the linker between BBM and the terminal amino group of A-5.

In certain embodiments, compounds of the present invention are generally prepared according to Scheme 4 set forth below:

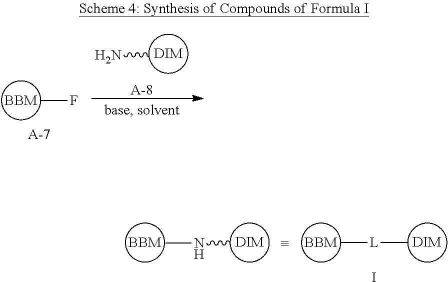

As depicted in Scheme 4, above, an S_{N}Ar displacement of fluoride A-7 by amine A-8 is effected in the presence of the base DIPEA in DMF to form a compound of formula I with a linker comprising a secondary amine. The squiggly bond, [symbol] , represents the portion of the linker between DIM and the terminal amino group of A-8.

In certain embodiments, compounds of the present invention are generally prepared according to Scheme 5 set forth below:

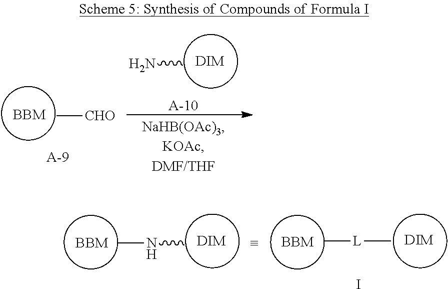

As depicted in Scheme 5, above, reductive amination of the mixture of aldehyde A-9 and amine A-10 is effected in the presence of NaHB(OAc)₃ and KOAc in DMF/THF to form a compound of formula I with a linker comprising a secondary amine. The squiggly bond, [symbol] , represents the portion of the linker between DIM and the terminal amino group of A-8.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the invention are described below in the Exemplification.

### 5. Uses, Formulation and Administration

### Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably degrade and/or inhibit BCL-XL and BCL-2 protein, or a mutant thereof, in a biological sample or in a patient. In certain embodiments, the amount of compound in compositions of this invention is such that is effective to measurably degrade and/or inhibit BCL-XL and BCL-2 protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitorily active metabolite or residue thereof” means that a metabolite or residue thereof is also an inhibitor of BCL-XL and BCL-2 protein, or a mutant thereof.

As used herein, the term “degratorily active metabolite or residue thereof” means that a metabolite or residue thereof is also a degrader of BCL-XL and BCL-2 protein, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water. Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic. pH adjusted sterile saline, or, preferably, as solutions in isotonic. pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### Uses of Compounds and Pharmaceutically Acceptable Compositions

Compounds and compositions described herein are generally useful for the degradation and/or inhibition of BCL-XL and BCL-2 protein activity.

As used herein, the terms “BCL-XL and BCL-2 mediated” disorders, diseases, and/or conditions as used herein means any disease or other deleterious condition in which BCL-XL and BCL-2, or mutants thereof, are known to play a role. Accordingly, another embodiment of the present invention relates to treating or lessening the severity of one or more diseases in which BCL-XL and BCL-2, or mutants thereof, are known to play a role. For example in some embodiments, the BCL-XL and BCL-2 mediated disorders, diseases, and/or conditions is cancer, autoimmune disease, or inflammation.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

The term “biological sample”, as used herein, includes, without limitation, cell cultures or extracts thereof: biopsied material obtained from a mammal or extracts thereof; and blood, saliva, urine, feces, semen, tears, or other body fluids or extracts thereof.

In some embodiments, the present invention provides a method of degrading BCL-XL and BCL-2 protein or mutants thereof in a biological sample. The method comprises contacting the biological sample with a therapeutically effective amount of a provided compound or a therapeutically acceptable salt thereof.

Inhibition and/or degradation of BCL-XL and BCL-2 protein or mutants thereof activity in a biological sample is useful for a variety of purposes that are known to one of skill in the art. Examples of such purposes include, but are not limited to, blood transfusion, organ-transplantation, biological specimen storage, and biological assays.

In some embodiments, the present invention provides a method of degrading BCL-XL and BCL-2 protein or mutants thereof in a patient in need thereof. The method comprises administering to the patient in need thereof a therapeutically effective amount of a provided compound or a therapeutically acceptable salt thereof.

In some embodiments, the present invention provides compounds that have pro-apoptotic properties. The ability to reactivate the apoptotic process in cells is of major therapeutic interest in the treatment of cancer, immune diseases, autoimmune diseases. In particular, the compounds according to the present invention are useful in the treatment of chemoresistant or radioresistant cancers. In another embodiment, the compounds of the invention may be used for treating diseases or conditions characterized by an excess or a deregulated activity of platelets, especially pro-thrombotic conditions.

In some embodiments, the present invention provides a method of killing one or more cancers in a patient in need thereof. The method comprises administering to the patient in need thereof a therapeutically effective amount of a provided compound or a therapeutically acceptable salt thereof. In some embodiments, the method of killing one or more cancers in a patient in need thereof comprises degradation BCL-XL, and BCL-2 protein or mutants thereof.

In some embodiments, the invention provides a method of treating cancer in a patient in need thereof comprising administering to the patient a therapeutically acceptable amount of a provided compound or a therapeutically acceptable salt thereof.

In some embodiments, the invention provides a method of selectively killing one or more cancer cells in a sample, the method comprising contacting the sample with an effective amount of a provided compound or a therapeutically acceptable salt thereof, or a pharmaceutically acceptable composition thereof. In another aspect, the present invention encompasses a method of selectively killing one or more cancer cells in a patient in need thereof, the method comprising administering to the patient in need thereof a provided compound or a therapeutically acceptable salt thereof, or a pharmaceutically acceptable composition thereof.

By selectively killing one or more cancer cells is meant a composition of the invention does not appreciably kill non-cancer cells at the same concentration. In some embodiments, a provided compound or a therapeutically acceptable salt thereof has reduced platelet toxicity and retained or improved toxicity in cancer cells when compared to BCL-XL and BCL-2 inhibitors.

In some embodiments, the method comprises killing one or more cancer cells in a patient with cancer. As such, the methods of the disclosure may be used to treat a tumor derived from a neoplasm or a cancer. The neoplasm may be malignant or benign, the cancer may be primary or metastatic; the neoplasm or cancer may be early stage or late stage. Non-limiting examples of neoplasms or cancers that may be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas, breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, choriocarcinoma, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, glioblastoma, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell renal pelvis transitional cell cancer, urethral cancer, uterine cancer (endometrial), uterine sarcoma, vaginal cancer, visual pathway and hypothalamic glioma (childhood), vulvar cancer. Waldenström macroglobulinemia, and Wilms tumor (childhood).

In certain embodiments, a cancer is selected from synovial sarcoma, Burkitt lymphoma, Hodgkin lymphoma, multiple myeloma, neuroblastoma, glioblastoma, small cell lung cancer, pancreatic cancer, hepatocellular (liver) cancer, endometrial cancer, ovarian cancer, cervical cancer, breast cancer, prostate cancer, bladder cancer, melanoma, rhabdomyosarcoma, osteosarcoma/malignant fibrous histiocytoma of bone, choriocarcinoma, kidney cancer (renal cell cancer), thyroid cancer, and leukemias (acute lymphoblastic, acute myeloid, chronic lymphocytic, and chronic myelogenous).

In some embodiments, the BCL-XL and BCL-2 mediated disorders, diseases, and/or conditions is an autoimmune disease. In some embodiments, the present invention provides a method of treating an autoimmune disease in a patient in need thereof, comprising administering to the patient a provided compound or a therapeutically acceptable salt thereof.

In some embodiments, the autoimmune disease is rheumatoid arthritis, psoriatic arthritis, juvenile idiopathic arthritis, multiple sclerosis, systemic lupus erythematosus, myasthenia gravis, juvenile onset diabetes, glomerulonephritis, autoimmune thyroiditis, Behcet's disease, Crohn's disease, ulcerative colitis, bullous pemphigoid, sarcoidosis, psoriasis, ichthyosis, Graves ophthalmopathy, psoriasis, psoriasis inflammatory bowel disease, or asthma.

In some embodiments, the BCL-XL and BCL-2 mediated disorders, diseases, and/or conditions is inflammation. In some embodiments, the present invention provides a method of treating inflammation in a patient in need thereof, comprising administering to the patient a provided compound or a therapeutically acceptable salt thereof.

In some embodiments, the inflammation is an inflammatory condition associated with lupus crythmatosus, psoriasis, psoriatic arthritis, lupus nephritis, rheumatoid arthritis, multiple sclerosis, ulcerative colitis, myasthenia gravis, immune thrombocytopeniarpura (ITP), thrombotic thrombocytopenia purpura (TTP), Grave's disease, Hashimoto's thyroiditis, Crohn's disease, autoimmune hemolytic anemias, insulin dependent diabetes mellitus, glomerulonephritis, rheumatic fever, osteoarthritis, gouty arthritis, dermatitis, bronchitis, rhinitis, asthma, Sjogrens' syndrome, meningitis, adrenoleukodystrophy, CNS vasculitis, mitochondrial myopathies, Amyotrophic Lateral Sclerosis, or Alzheimer's disease.

As discussed in more detail herein, some embodiments provide methods for treating and/or prevention of various inflammatory disorders, diseases and conditions. Such inflammatory disorders, diseases and conditions include, without limitation, systemic autoimmune diseases such as, for example, lupus erythematosus, rheumatoid arthritis, multiple sclerosis, and psoriasis; and organ specific autoimmune diseases such as, for example, ulcerative colitis, myasthenia gravis, Grave's disease, Hashimoto's thyroiditis, Crohn's disease, lupus nephritis, autoimmune hemolytic anemias, ITP, TTP, insulin dependent diabetes mellitus, glomerulonephritis, and rheumatic fever. Other inflammatory diseases that may be treated in accordance with this disclosure include, without limitation, other inflammatory arthritic conditions such as psoriatic arthritis, osteoarthritis and gouty arthritis, as well as other inflammatory conditions such as conjunctivitis, dermatitis, bronchitis, rhinitis etc., brought about by injury, allergies, infections, microorganisms, trauma, or physical or chemical agents. The treatment of inflammatory aspects of asthma, Sjogrens' syndrome, meningitis, adrenoleukodystrophy, CNS vasculitis, mitochondrial myopathies, Amyotrophic Lateral Sclerosis, Alzheimer's disease, or tumors is also contemplated as part of this disclosure. Examples of mitochondrial myopathies include MELAS syndrome, MERF syndrome, Leber's disease, Wernicke's encephalopathy. Rett syndrome, homocystinuria, hyperprolinemia, nonketotic hyperglycinemia, hydroxybutyric aminoaciduria, sulfite oxidase deficiency, and combined systems disease (B 12 deficiency). In association with such prevention and/or treatment, articles of manufacture, compositions, methods of use, and medical treatments by the compounds described herein are also provided.

In some embodiments, a provided compound is an anti-senolytic agent. Accordingly in some embodiments, the present invention provides a method of treating an age-related disease including various fibrotic diseases, osteoarthritis, and kidney diseases. In some embodiments, the age-related disease is diabetes, obesity, cardiac dysfunction, vascular hyporeactivity/calcification, AV fistulae, frailty, age-related muscle loss (sarcopenia), chemotherapy complication, radiation complication, cancer, bone marrow transplant complication, organ transplantation complication, myeloma/monoclonal gammopathy of undetermined significance (MGUS), age-related cognitive dysfunction, Alzheimer's disease, Parkinson's disease, or amyotrophic lateral sclerosis. See e.g., Kirkland and Tchkonia. “Senolytic drugs: From discovery to translation.” Journal of Internal Medicine 2020, 288 (5):518-536.

It is believed that a provided compound or a pharmaceutically acceptable salt thereof may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

Co-Administration with One or More Other Therapeutic Agent(s)

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination.” “combined.” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours apart.

In one embodiment, the present invention provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-II, -I” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclominc (Bentyl®), Singulair®, beta-2 agonists such as albuterol (Ventolin® HFA, Proventil®; HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Acrobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating gout comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmunc®), leflunomide (Arava®) and “anti-TNF” agents such as etanercept (Enbrelk®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil®, HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Acrobid®), Afviar®, Symbicort®, and Dulera®, cromolyn sodium (Intal®), methylxanthines such as theophylline (Theo-Dur®, Thcolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Acrobid®), Afviar®, Symbicort®, and Dulera®.

In some embodiments, the present invention provides a method of treating HIV comprising administering to a patient in need thereof a provided compound and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In another embodiment, the present invention provides a method of treating a hematological malignancy comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a provided compound and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a provided compound and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenström's macroglobulinemia comprising administering to a patient in need thereof a provided compound and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®), Celgene); panobinostat (Farydak®, Novartis); belinostat (Beleodaq®), Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®), Pfizer); ribociclib (Kisqali®, Novartis); abemaciclib (Ly2835219, Eli Lilly); and trilaciclib (GIT28, G1 Therapeutics).

In some embodiments, one or more other therapeutic agent is a folic acid inhibitor. Approved folic acid inhibitors useful in the present invention include pemetrexed (Alimta®), Eli Lilly).

In some embodiments, one or more other therapeutic agent is a CC chemokine receptor 4 (CCR4) inhibitor, CCR4 inhibitors being studied that may be useful in the present invention include mogamulizumab (Poteligeo®), Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®), GlaxoSmithKline); obinutuzumab (anti-CD20, Gazyvak, Genentech), ibritumomab (anti-CD20 and Yttrium-90, Zevalink, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Scattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech) and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, the present invention provides a method of treating cancer (e.g., leukemias, lymphomas, etc.) comprising administering to a patient in need thereof a provided compound and one or more BCL-2 inhibitors.

In some embodiments, one or more other therapeutic agent is a MCL-1 inhibitor. MCL-1 inhibitors which may be used in the present invention include marinopyrole A (Maritoclax), VU661013, AZD5991, S63845, S64315 (MIK665), A-1210477, and UMI-77.

In some embodiments, the present invention provides a method of treating cancer (e.g., leukemias, lymphomas, etc.) comprising administering to a patient in need thereof a provided compound and one or more MCL-1 inhibitors.

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFß). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFß trap compound (NCT02699515); and (NCT02517398). M7824 is comprised of a fully human IgG1 antibody against PD-L1 fused to the extracellular domain of human TGF-beta receptor II, which functions as a TGFß “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE, gpNMB is a protein overexpressed by multiple tumor types associated with cancer cells ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics: temozolomide (Temodal®); kinesin spindle protein inhibitors, such as SB715992 or SB743921 from GlaxoSmithKline, or pentamidine/chlorpromazine from CombinatoRx; MEK inhibitors such as selumetinib (Kosclugo®) from Array BioPharma, AZd₆244 from AstraZeneca, PD181461 from Pfizer and leucovorin.

In some embodiments, the present invention provides a method of treating Alzheimer's disease comprising administering to a patient in need thereof a provided compound and one or more additional therapeutic agents selected from donepezil (Aricept®), rivastigmine (Excelon®), galantamine (Razadyne®), tacrine (Cognex®), and memantine (Namenda®).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CecNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Erwinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi); VEGFR inhibitors, such as regorafenib (Stivarga®, Bayer), vandetanib (Caprelsa®, AstraZeneca), axitinib (Inlyta®, Pfizer), and lenvatinib (Lenvima®, Eisai); Raf inhibitors, such as sorafenib (Nexavar®, Bayer AG and Onyx), dabrafenib (Tafinlar®, Novartis), and vemurafenib (Zelboraf®, Genentech/Roche); MEK inhibitors, such as selumetinib (Kosclugo®, AstraZeneca), binimetinib (Mektovi®, Array BioPharma), cobimetanib (Cotellic®, Exelexis/Genentech/Roche), and trametinib (Mekinist®, Novartis); Bcr-Abl tyrosine kinase inhibitors, such as imatinib (Gleevec®, Novartis), nilotinib (Tasigna®, Novartis), dasatinib (Sprycel®, BristolMyersSquibb), bosutinib (Bosulif®, Pfizer), and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as lenvatinib (Lenvima®, Easai), sunitinib (Sutent®, Pfizer), pontatinib (Iclusig®, Ariad), and pazopanib (Votrient®, Novartis); ALK inhibitors such as crizotinib (Xalkori®, Pfizer), ceritinib (Zykadia®, Novartis), brigatinib (Alunbrig®, Ariad), lorlatinib (Lorbrena®, Pfizer), and alectinib (Alecenza®, Genentech/Roche); Bruton's tyrosine kinase inhibitors, such as ibrutinib (Imbruvica®, Pharmacyclics/Janssen); Flt3 receptor inhibitors, such as midostaurin (Rydapt®, Novartis) and gilteritinib (Xospata®, Astellas); FGFR inhibitors such as pemigatinib (Pemazyre®, Incyte), nintedanib (Ofev®, Bochringer Ingelheim), and erdafitinib (Balversa®, Janssen Pharmaceuticals); RET inhibitors such as selpercatinib (Retivmo®, Eli Lilly), pralsctinib (Gavreto®, Blueprint Medicines); KIT inhibitors such as ripretinib (Qinlock®, Deciphera Pharmaceuticals), and avapritinib (Ayvakit®, Blueprint Medicines).

In some embodiments, the present invention provides a method of treating cancer comprising administering to a patient in need thereof a provided compound and one or more additional therapeutic agents selected from tyrosine kinase inhibitors or RAS-RAF-MEK-ERK pathway inhibitors.

In some embodiments, the present invention provides a method of treating cancer comprising administering to a patient in need thereof a provided compound and one or more FGFR inhibitors.

In some embodiments, the present invention provides a method of treating cancer comprising administering to a patient in need thereof a provided compound and one or more RET inhibitors.

In some embodiments, the present invention provides a method of treating cancer comprising administering to a patient in need thereof a provided compound and one or more KIT inhibitors.

In some embodiments, the present invention provides a method of treating cancer comprising administering to a patient in need thereof a provided compound and one or more ALK inhibitors.

In some embodiments, the present invention provides a method of treating cancer comprising administering to a patient in need thereof a provided compound and one or more RAS inhibitors.

In some embodiments, the present invention provides a method of treating cancer comprising administering to a patient in need thereof a provided compound and one or more MEK inhibitors.

Other kinase inhibitors and VEGF-R antagonists that are in development and may be used in the present invention include tivozanib (Aveo Pharmaceuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs. host disease comprising administering to a patient in need thereof a provided compound and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenia purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenia purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis. Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder. (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenia states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenia purpura, Waldenstrom macroglobulinemia, myasthenia gravis. Hashimoto's thyroiditis, atopic dermatitis, degenerative joint disease, vitiligo, autoimmune hypopituitarism, Guillain-Barre syndrome, Behcet's disease, scleroderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound and a PI3K inhibitor, wherein the disease is selected from a cancer, a neurodegenerative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, scleredoma, Wegener granulomatosis, dermatomyositis, chronic active hepatitis, myasthenia gravis. Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure. Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™ Letrozole is marketed under the trade names Femara™ or Femar™ Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™ Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate, Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caclyx™), daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed, under the trade name Zavedos™, Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel: vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “BCL-2 inhibitor” as used herein includes, but is not limited to compounds having inhibitory activity against B-cell lymphoma 2 protein (BCL-2), including but not limited to ABT-199, ABT-731, ABT-737, apogossypol, Ascenta's pan-BCL-2 inhibitors, curcumin (and analogs thereof), dual BCL-2/BCL-XL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the BCL-2 inhibitor is a small molecule therapeutic. In some embodiments the BCL-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; Ilmofosine; RO 318220 and RO 320432; GO 6976; Isis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); l) compounds targeting, decreasing or inhibiting the activity of the epidermal growth factor family of receptor tyrosine kinases (EGFR1 ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idclalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib. XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806. WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™, Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™),); carfilzomib (Kyprolis®, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino, 17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4th Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl) phthalazine or a pharmaceutically acceptable salt thereof. 1-(4-chloroanilino)-4-(4-pyridylmethyl) phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTBR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin aIβ2, FAS, FASL, RELT, DR6, TROY, NGFR.

In some embodiments, an immuno-oncology agent is a cytokine that inhibits T cell activation (e.g., IL-6, IL-10, TGF-B, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2.3)-dioxygenase (IDO) antagonist. In some embodiments, an IDO antagonist is selected from cpacadostat (INCB024360, Incytc); indoximod (NLG-8189, New Link Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygick, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (Pexa Vec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil: TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8 T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15; sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood. “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLOS ONE 12 (8): e0183390, the contents of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands, B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM. TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736). MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675.206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma, AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453, TSR-022 (Tesaro) is an anti-TIM-3 antibody which is being studied in solid tumors (NCT02817633), LY3321367 (Eli Lilly) is an anti-TIM-3 antibody which is being studied in solid tumors (NCT03099109), MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321, BMS-986016 (Bristol-Myers Squibb), an anti-LAG-3 antibody, is being studied in glioblastoma and gliosarcoma (NCT02658981), REGN3767 (Regeneron), is also an anti-LAG-3 antibody, and is being studied in malignancies (NCT03005782), IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgGI, acts by binding human CD47, and preventing it from delivering its “do not cat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

In some embodiments, stat3 inhibition/degradation can significantly enhance CDN-induced STING signaling and antitumor immunity (Pei et al., Can. Lett. 2019, 450:110).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### Abbreviations

Ac: acetyl
AcOH: acetic acid
ACN: acetonitrile
Ad: adamantly
AIBN: 2,2′-azo bisisobutyronitrile
Anhyd: anhydrous
Aq: aqueous
B₂Pin₂: bis(pinacolato)diboron-4,4,4′,4′,5,5,5′,5′-octamethyl-2,2′-bi (1,3,2-dioxaborolane)
BINAP: 2,2′-bis(diphenylphosphino)-1,1′-binaphthyl
BH₃: borane
Bn: benzyl
Boc: tert-butoxycarbonyl
Boc₂O: di-tert-butyl dicarbonate
BPO: benzoyl peroxide
ⁿBuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DPPA: diphenylphosphoryl azide
dppf: 1,1′-bis(diphenylphosphino) ferrocene
EDC or EDCI: 1-(3-dimethylaminopropyl)-3-ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-yl) uranium hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
m-CPBA: meta-chloroperbenzoic acid
M: molar
MeCN: acetonitrile
MeOH: methanol
Me₂S: dimethyl sulfide
MeONa: sodium methylate
MeI: iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-methylmorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
PPh₃: triphenylphosphine
PyBOP: (Benzotriazol-1-yloxy)tripyrrolidinophosphonium hexafluorophosphate
Rel: relative
R.T. or rt: room temperature
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAI: tetrabutylammonium iodide
TEA: triethylamine
Tf: trifluoromethanesulfonate
TfAA, TFMSA or Tf₂O: trifluoromethanesulfonic anhydride
TFA: trifluoracetic acid
TIPS: triisopropylsilyl
THF: tetrahydrofuran
THP: tetrahydropyran
TLC: thin layer chromatography
TMEDA: tetramethylethylenediamine
pTSA: para-toluenesulfonic acid
wt: weight
Xantphos: 4,5-bis(diphenylphosphino)-9,9-dimethylxanthene

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

TABLE 2
Analytical instruments

| | |
|---|---|
| LCMS | Shimadzu UFLC MS: LCMS-2020 |
| | Agilent Technologies 1200 series MS: |
| | Agilent Technologies 6110 |
| | Agilent Technologies 1200 series MS: |
| | LC/MSD VL |
| NMR | BRUKER AVANCE III/400; Frequency (MHz) |
| | 400.13; Nucleus: 1H; Number of Transients: 8 |
| Prep-HPLC | Gilson GX-281 systems: instruments |
| | GX-A, GX-B, GX-C, GX-D, GX-E, |
| | GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH⁺] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH⁺] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH₃·H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19) mm, 5μ. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

The synthesis of Intermediates A, D, C, E, F, O, P, W, AF, AT, AW, AX, AZ, BA, BB, BK, BL, BO, CB, CK, DN, DP, and IG may be carried out as described in WO2023044046, the entirety of which are incorporated herein by reference.

### Example 1: Synthesis of Intermediates of Compounds of Formula I

### Synthesis of (2S,4R)-1-((S)-2-amino-3,3-dimethylbutanoyl)-N—((S)-1-(4-ethynylphenyl)ethyl)-4-hydroxypyrrolidine-2-carboxamide (Intermediate PZ) and Phenyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)carbamate (Intermediate BM)

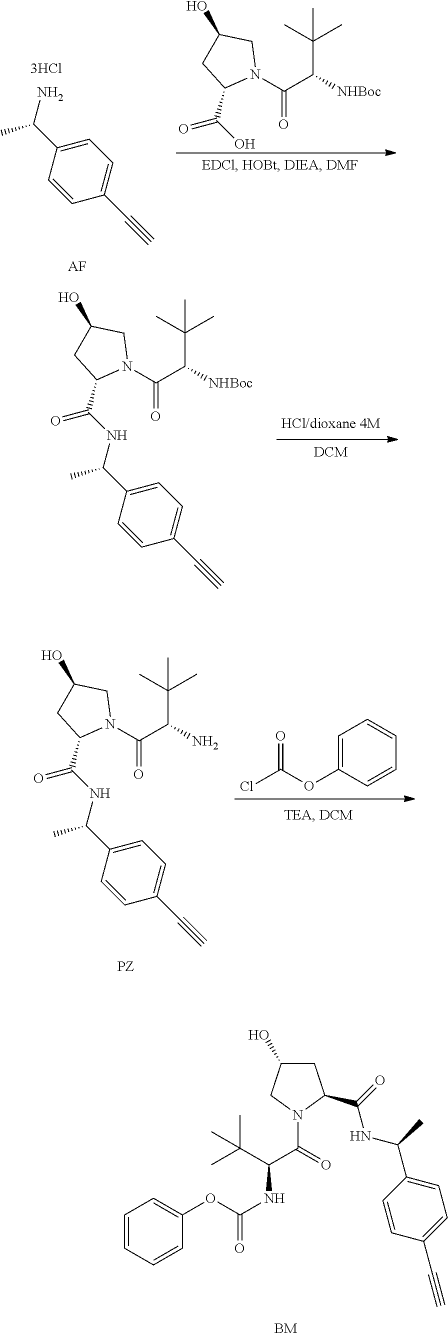

### Step 1—Tert-butyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)carbamate

To a solution of Tert-butyl (S)-(1-(4-ethynylphenyl)ethyl)carbamate (47.7 g, 187 mmol, 3 HCl, Intermediate AF) in DMF (400 mL) was added DIEA (66.7 g, 516 mmol) and the mixture was stirred at 25° C. for 0.25 hrs. Then to the mixture was added (2S,4R)-1-((S)-2- ((tert-butoxycarbonyl)amino)-3,3-dimethylbutanoyl)-4-hydroxypyrrolidine-2-carboxylic acid (43.0 g, 124 mmol), EDCI (28.7 g, 149 mmol) and HOBt (20.2 g, 149 mmol), then the mixture was stirred at 25° C. for 3 hrs. On completion, the mixture was poured into water (3.00 L), and extracted with EtOAc (1.00 L×4). The organic layer was washed with water (2.00 L×4), brine (1.50 L), dried over Na₂SO₄, filtered and concentrated. The residue was purified by silica gel chromatography eluted with Petroleum ether/Ethyl acetate (1/0 to 0/1) to give the title compound (45.0 g) as a light yellow solid. LC-MS (ESI⁺) m/z 472.3 (M+H)⁺. ¹H NMR (400 MHz, CDCl₃) δ 7.37 (br d, J=7.70 Hz, 1H), 7.27 (s, 2H), 7.08 (d, J=8.07 Hz, 2H), 5.07 (br d, J=9.05 Hz, 1H), 4.84 (quin, J=7.06 Hz, 1H), 4.52 (t, J=7.89 Hz, 1H), 4.29 (br s, 1H), 4.02 (d, J=9.17 Hz, 1H), 3.84 (br d, J=11.25 Hz, 1H), 3.24-3.35 (m, 2H), 2.86 (s, 1H), 2.19-2.33 (m, 1H), 1.94 (s, 1H), 1.18-1.30 (m, 12H), 0.83 (s, 9H).

### Step 2—(2S,4R)-1-((S)-2-amino-3,3-dimethylbutanoyl)-N—((S)-1-(4-ethynylphenyl)ethyl)-4-hydroxypyrrolidine-2-carboxamide

To a solution of Tert-butyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)carbamate (40.0 g, 84.8 mmol) in DCM (400 mL) was added HCl/dioxane (4 M, 26.0 mL) at 0-10° C., then the mixture was stirred at 10-20° C. for 16 hrs. On completion, the mixture was added dropwise into n-heptane (2.00 L). The precipitate was then filtered to give the filter cake. The compound was then dissolved in DCM (400 mL), and dissociated with basic resin (200 g) by stirring at 20° C. for 2 hrs. The mixture was filtered and concentrated to give a residue. The residue was purified by prep-HPLC(NH₃·H₂O condition) and freeze-dried to give the title compound (19.8 g, 97% yield) as a white solid. LC-MS (ESI⁺) m/z 372.1 (M+H)⁺. ¹H NMR (400 MHz, CDCl₃) δ 7.73 (br d, J=6.40 Hz, 1H), 7.46 (d, J=8.16 Hz, 2H), 7.27 (s, 2H), 5.04 (br t, J=7.15 Hz, 1H), 4.74 (br t, J=7.72 Hz, 1H), 4.46 (br s, 1H), 3.75 (br d, J=10.92 Hz, 1H), 3.59 (br dd, J=11.04, 3.76 Hz, 1H), 3.42 (br s, 1H), 3.07 (s, 1H), 2.36 (br d, J=5.65 Hz, 1H), 2.04-2.15 (m, 1H), 1.45 (d, J=6.90 Hz, 3H), 1.02 (s, 9H).

### Step 3—Phenyl ((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)carbamate

To a solution of (2S,4R)-1-[(2S)-2-amino-3,3-dimethyl-butanox]-N-[(1S)-1-(4-ethynylphenyl)ethyl]-4-hydroxy-pyrrolidine-2-carboxamide (50 mg, 134 μmol) and phenyl carbonochloridate (21.0 mg, 134 μmol) in DCM (0.5 mL) was added TEA (40.8 mg, 403 μmol). The mixture was stirred at 25° C. for 2 hr. On completion, the mixture was quenched by H₂O (5 mL) and extracted with EtOAc (5 mL×3). The combined organic layers were washed with brine (5 mL×2), dried over Na₂SO₄, filtered and concentrated under reduced pressure to give the title compound (60 mg) as a white solid. LC-MS (ESI⁺) m/z 492.4 (M+H)⁺.

### Synthesis of (2S,4R)-methyl 1-((S)-2-amino-3,3-dimethylbutanoyl)-4-hydroxypyrrolidine-2-carboxylate (Intermediate CH)

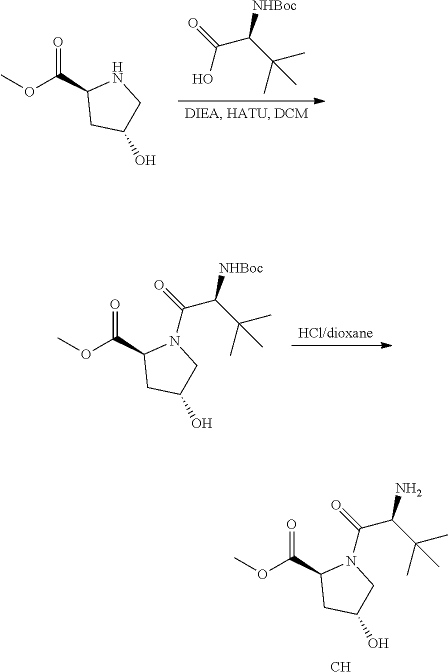

### Step 1—(2S,4R)-methyl1-((S)-2-((tert-butoxycarbonyl)amino)-3,3-dimethylbutanoyl)-4-hydroxypyrrolidine-2-carboxylate

To a solution of (2S,4R)-methyl 4-hydroxypyrrolidine-2-carboxylate (10.0 g, 68.9 mmol. CAS #1499-56-5), (S)-2-((tert-butoxycarbonyl)amino)-3,3-dimethylbutanoic acid (15.9 g, 68.9 mmol, CAS #62965-35-9) in DCM (200 mL) was added HATU (39.3 g, 103 mmol) and DIEA (44.5 g, 344 mmol). The mixture was stirred at 25° C. for 12 h. On completion, the reaction mixture was partitioned between solvent DCM (100 mL×2) and H₂O (20 mL×2). The organic phase was separated, washed with brine (100 mL×2), dried over Na₂SO₄, filtered and concentrated under reduced pressure to give a residue. The residue was purified by column chromatography (SiO₂, Petroleum ether/Ethyl acetate=20/1 to 0/1) to give the title compound (20.0 g, 81% yield) as a brown oil. LC-MS (ESI⁺) m/z 359.0 (M+H)⁺.

### Step 2—(2S,4R)-methyl 1-((S)-2-amino-3,3-dimethylbutanoyl)-4-hydroxypyrrolidine-2-carboxylate

To a solution of (2S,4R)-methyl 1-((S)-2-((tert-butoxycarbonyl)amino)-3,3-dimethylbutanoyl)-4-hydroxypyrrolidine-2-carboxylate (20.0 g, 55.8 mmol) in DCM (40 mL) was added dropwise HCl/dioxane (4 M, 37.8 mL). The resulting mixture was stirred at 20° C. for 5 h. On completion, the reaction mixture was evaporated to give the title compound (17.0 g) as a white solid. LC-MS (ESI⁺) m/z 259.1 (M+H)⁺.

### Synthesis of (2S,4R)-1-((S)-2-(1-fluorocyclopropanecarboxamido)-3,3-dimethylbutanoyl)-4-hydroxypyrrolidine-2-carboxylic Acid (Intermediate CI)

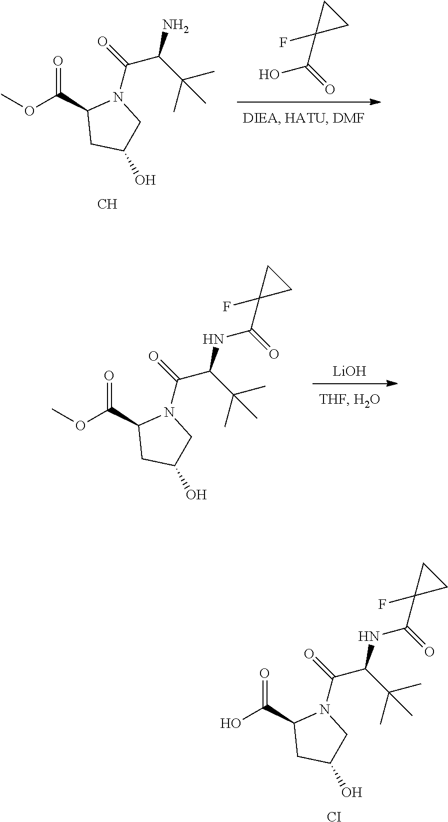

### Step 1—(2S,4R)-methyl 1-((S)-2-(1-fluorocyclopropanecarboxamido)-3,3-dimethylbutanoyl)-4-hydroxypyrrolidine-2-carboxylate

To a solution of (2S,4R)-methyl 1-((S)-2-amino-3,3-dimethylbutanoyl)-4-hydroxypyrrolidine-2-carboxylate (17.0 g, 57.7 mmol, HCl salt, Intermediate CH) and 1-fluorocyclopropanecarboxylic acid (6.00 g, 57.7 mmol) in DMF (170 mL) was added HATU (26.3 g, 69.2 mmol) and DIEA (74.5 g, 577 mmol). The mixture was stirred at 25° C. for 12 h. On completion, the reaction mixture was partitioned between solvent EtOAc (500 mL×4) and H₂O (250 mL). The organic phase was separated, washed with brine (100 mL×5), dried over Na₂SO₄, filtered and concentrated under reduced pressure to give a residue. The crude product was purified by column chromatography (SiO₂, Petroleum ether/Ethyl acetate=5/1 to 3/1) to give the title compound (11.0 g, 55% yield) as a brown oil. LC-MS (ESI⁺) m/z 345.2 (M+H)⁺.

### Step 2—(2S,4R)-1-((S)-2-(1-fluorocyclopropanecarboxamido)-3,3-dimethylbutanoyl)-4-hydroxypyrrolidine-2-carboxylic Acid

To a solution of (2S,4R)-methyl 1-((S)-2-(1-fluorocyclopropanecarboxamido)-3,3-dimethylbutanoyl)-4-hydroxypyrrolidine-2-carboxylate (1.00 g, 2.90 mmol) in THF (10 mL) was added LiOH·H₂O (366 mg, 8.71 mmol) in H₂O (5 mL). The mixture was stirred at 25° C. for 2 h. On completion, the pH of the reaction mixture was adjusted to 4˜3 by addition 2 M HCl, then the mixture was extracted with EtOAc (10 mL×10). The organic phase was separated, dried over Na₂SO₄, filtered and concentrated under reduced pressure to give the title compound (720 mg) as a white oil.

### Synthesis of (S)-2-(1-aminoethyl)-5-(4-methylthiazol-5-yl)phenol (Intermediate DV)

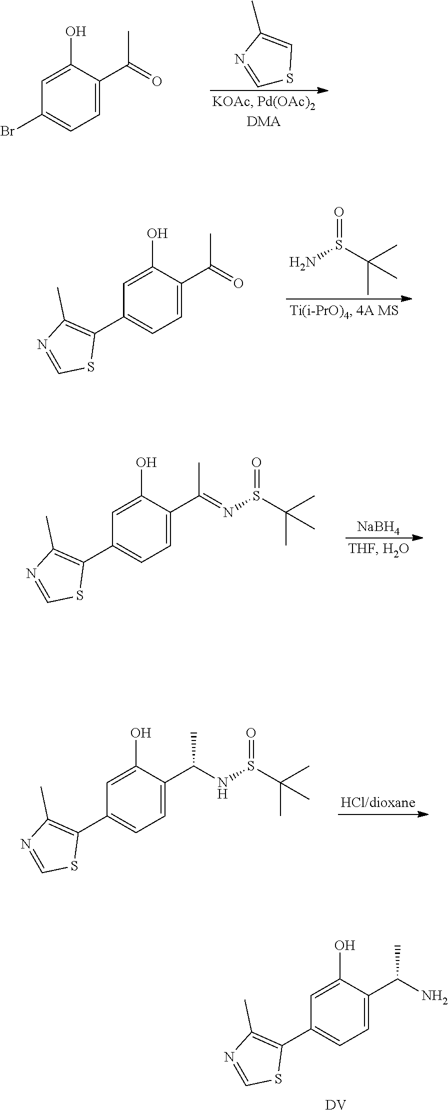

### Step 1—1-(2-Hydroxy-4-(4-methylthiazol-5-yl)phenyl)ethanone

To a solution of 1-(4-bromo-2-hydroxy-phenyl)ethanone (20.0 g, 93.0 mmol), 4-methylthiazole (18.4 g, 186 mmol) and KOAc (18.3 g, 186 mmol) in DMA (140 mL) was added Pd(OAc)₂ (1.04 g, 4.65 mmol) under N₂. Then the mixture was stirred at 120° C. for 12 h. On completion, the reaction mixture was poured into ice water (300 mL) and extracted with ethyl acetate (300 mL×3). The combined organic layers were washed with brine (100 mL×5), dried over sodium sulfate, filtered and concentrated under reduced pressure to give a residue. The residue was purified by column chromatography (SiO₂, Petroleum ether/Ethyl acetate=40/1 to 10/1) to afford title compound (20.0 g, 92% yield) as a yellow solid. ¹H NMR (400 MHz, CHLOROFORM-d) δ=12.37 (s, 1H), 8.74 (s, 1H), 7.79 (d, J=8.4 Hz, 1H), 7.10 (d, J=1.6 Hz, 1H), 7.01 (dd, J=1.6, 8.4 Hz, 1H), 2.67 (s, 3H), 2.61 (s, 3H).

### Step 2—(R,E)-N-(1-(2-hydroxy-4-(4-methylthiazol-5-yl)phenyl)ethylidene)-2-methylpropane-2-sulfinamide

To a solution of 1-(2-hydroxy-4-(4-methylthiazol-5-yl)phenyl)ethanone (500 mg, 2.14 mmol), (R)-2-methylpropane-2-sulfinamide (779 mg, 6.43 mmol) and 4 Å molecular sieves (500 mg) in 2-methyltetrahydrofuran (10 mL) was added Ti(i-PrO)₄ (1.83 g, 6.43 mmol) at 0° C. under N₂. Then the mixture was heated to 80° C. for 2 h. On completion, the reaction mixture was diluted with EtOAc (50 mL) and H₂O (10 mL) and filtered. The mixture was diluted with water (10 mL), and extracted with EtOAc (20 mL×3). The combined organic layers were washed with brine (20 mL×3), dried over Na₂SO₄ and evaporated. The residue was purified by column chromatography (SiO₂, Petroleum ether/Ethyl acetate=3/1 to 2/1) to afford title compound (4 g, 56% yield) as a yellow solid. LC-MS (ESI⁺) m/z 337.0 (M+H)⁺.

### Step 3—(R)—N—((S)-1-(2-hydroxy-4-(4-methylthiazol-5-yl)phenyl)ethyl)-2-methylpropane-2-sulfinamide

To a solution of (R,E)-N-(1-(2-hydroxy-4-(4-methylthiazol-5-yl)phenyl)ethylidene)-2-methylpropane-2-sulfinamide (4.00 g, 11.9 mmol) in THF (39.2 mL) and H₂O (0.8 mL) was added NaBH₄ (1.85 g, 48.9 mmol) at −50° C. under N₂. The mixture was stirred at 25° C. for 12 h and 40° C. for 12 h. On completion, the reaction mixture was quenched with H₂O (50 mL), and extracted with EtOAc (50 mL×3). The combined organic layers were washed with brine (40 mL×3), dried over Na₂SO₄ and evaporated. The residue was purified by prep-HPLC (column: Phenomenex luna C18 250*50 mm*15 um; mobile phase: [water (NH3H2O)-ACN]; B %: 15%-55%, 21 min) and reversed-phase HPLC (ACN/0.1% NH₃·H₂O=50%) to afford title compound (1.3 g, 32% yield) as a white solid. ¹H NMR (400 MHz, CHLOROFORM-d) δ=8.65 (s, 1H), 8.45 (br s, 1H), 7.20 (d, J=8.0 Hz, 1H), 7.00 (d, J=1.6 Hz, 1H), 6.92 (dd, J=1.6, 8.0 Hz, 1H), 4.65 (dd, J=5.2, 6.8 Hz, 1H), 4.14 (d, J=5.2 Hz, 1H), 2.51 (s, 3H), 1.62 (d, J=6.8 Hz, 3H), 1.28 (s, 9H).

### Step 4—(S)-2-(1-aminoethyl)-5-(4-methylthiazol-5-yl)phenol

To a solution of (R)—N—((S)-1-(2-hydroxy-4-(4-methylthiazol-5-yl)phenyl)ethyl)-2-methylpropane-2-sulfinamide (1.10 g, 3.25 mmol) in dioxane (1.2 mL) was added HCl/dioxane (4 M, 11.0 mL). The mixture was stirred at 25° C. for 1 h. On completion, the reaction mixture was evaporated. The crude product was purified by reversed-phase HPLC (ACN/0.1% HCl=0%) to afford title compound (550 mg, HCl salt) as a white solid. LC-MS (ESI⁻) m/z 235.1 (M+H)⁺.

### Synthesis of 2-(2-((S)-1-((2S,4R)-1-((S)-2-(1-fluorocyclopropanecarboxamido)-3,3-dimethylbutanoyl)-4-hydroxypyrrolidine-2-carboxamido)ethyl)-5-(4-methylthiazol-5-yl)phenoxy)acetic Acid (Intermediate DW)

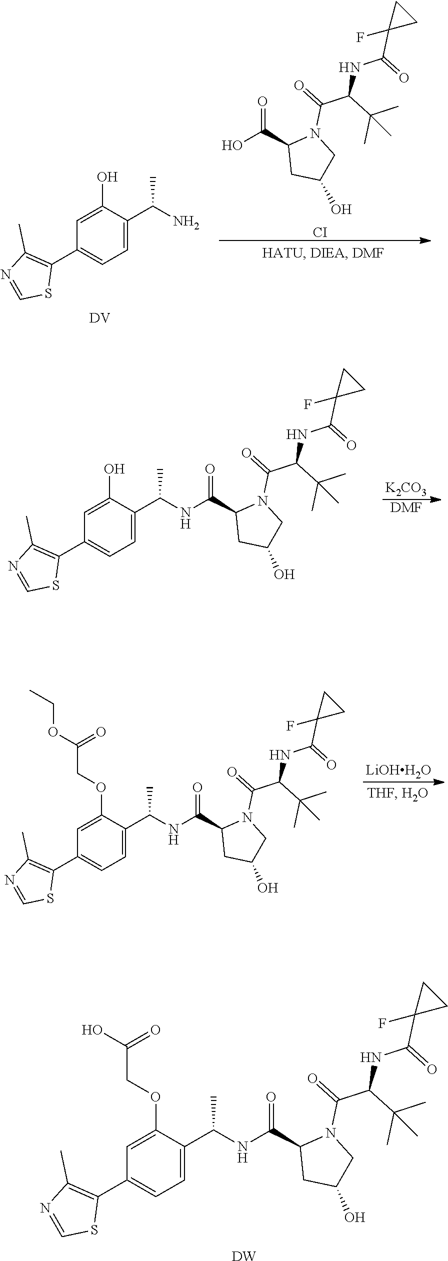

### Step 1—(2S,4R)-1-((S)-2-(1-fluorocyclopropanecarboxamido)-3,3-dimethylbutanoyl)-4-hydroxy-N—((S)-1-(2-hydroxy-4-(4-methylthiazol-5-yl)phenyl)ethyl)pyrrolidine-2-carboxamide

A solution of (2S,4R)-1-((S)-2-(1-fluorocyclopropanecarboxamido)-3,3-dimethylbutanoyl)-4-hydroxypyrrolidine-2-carboxylic acid (1.03 g, 3.12 mmol, Intermediate CI) and HATU (1.37 g, 3.60 mmol) in DMF (6.5 mL) was stirred for 0.5 h at 25° C. Then a mixture of DIEA (1.55 g, 12.0 mmol) and (S)-2-(1-aminoethyl)-5-(4-methylthiazol-5-yl)phenol (650 mg, 2.40 mmol, HCl salt, Intermediate DV) in DMF (3.2 mL) was added. The mixture was stirred at 25° C. for 12 h. On completion, the reaction mixture was purified by reversed-phase HPLC without work up. The crude product was purified by reversed-phase HPLC (ACN/0.1% FA condition=50% to 60%) to afford title compound (950 mg, 72% yield) as a yellow solid, ¹H NMR (400 MHz, DMSO-d₆) δ=9.75 (s, 1H), 8.94 (s, 1H), 8.43 (d, J=7.6 Hz, 1H), 8.13 (s, 1H), 7.48 (d, J=8.0 Hz, 1H), 7.27 (br dd, J=2.0, 9.2 Hz, 1H), 6.89 (d, J=1.6 Hz, 1H), 6.84-6.78 (m, 1H), 5.15 (d, J=3.6 Hz, 1H), 4.96 (t, J=7.2 Hz, 1H), 4.60-4.51 (m, 2H), 4.34 (br s, 1H), 3.63-3.55 (m, 2H), 2.44 (s, 3H), 2.09-2.01 (m, 1H), 1.92 (ddd, J=4.4, 8.4, 12.8 Hz, 1H), 1.39 (br d, J=8.8 Hz, 1H), 1.31 (d, J=6.8 Hz, 3H), 1.21 (br dd, J=3.6, 8.8 Hz, 2H), 0.96 (br d, J=6.8 Hz, 1H), 0.89 (s, 9H).

### Step 2—Ethyl 2-(2-((S)-1-((2S,4R)-1-((S)-2-(1-fluorocyclopropanecarboxamido)-3,3-dimethylbutanoyl)-4-hydroxypyrrolidine-2-carboxamido)ethyl)-5-(4-methylthiazol-5-yl)phenoxy)acetate

To a solution of (2S,4R)-1-((S)-2-(1-fluorocyclopropanecarboxamido)-3,3-dimethylbutanoyl)-4-hydroxy-N—((S)-1-(2-hydroxy-4-(4-methylthiazol-5-yl)phenyl)ethyl)pyrrolidine-2-carboxamide (200 mg, 366 μmol) and K₂CO₃ (152 mg, 1.10 mmol) in DMF (2 mL) was added ethyl 2-bromoacetate (91.7 mg, 549 μmol). Then the mixture was stirred at 60° C. for 12 h. On completion, the reaction mixture was diluted with water (10 mL), and extracted with EtOAc (15 mL×3). The combined organic layers were washed with brine (5 mL×4), dried over Na₂SO₄ and evaporated to afford the title compound (240 mg) as a yellow oil. LC-MS (ESI⁺) m/z 633.3 (M+H)⁺.

### Step 3—2-(2-((S)-1-((2S,4R)-1-((S)-2-(1-fluorocyclopropanecarboxamido)-3,3-dimethylbutanoyl)-4-hydroxypyrrolidine-2-carboxamido)ethyl)-5-(4-methylthiazol-5-yl)phenoxy)acetic Acid

To a solution of ethyl 2-(2-((S)-1-((2S,4R)-1-((S)-2-(1-fluorocyclopropanecarboxamido)-3,3-dimethylbutanoyl)-4-hydroxypyrrolidine-2-carboxamido)ethyl)-5-(4-methylthiazol-5-yl)phenoxy)acetate (240 mg, 379 μmol) in THF (2 mL) and H₂O (1 mL) was added LiOH·H₂O (53.0 mg, 1.26 mmol). Then the mixture was stirred at 25° C. for 2 h. On completion, the reaction mixture was diluted with water (5 mL) and the pH was adjusted to 4˜3 by addition 2 M HCl, then extracted with EtOAc (10 mL×3). The combined organic layers were washed with brine (4 mL×3), dried over Na₂SO₄ and evaporated to afford the title compound (200 mg) as a yellow oil. ¹H NMR (400 MHz, DMSO-d₆) β=13.29-12.88 (m, 1H), 8.97 (s, 1H), 8.49 (br d, J=7.6 Hz, 1H), 7.57 (d, J=7.6 Hz, 1H), 7.30-7.26 (m, 1H), 6.96 (d, J=8.0 Hz, 1H), 6.90 (s, 1H), 5.03 (br t, J=6.8 Hz, 1H), 4.81 (d, J=5.2 Hz, 2H), 4.58-4.53 (m, 2H), 4.37-4.33 (m, 1H), 3.61-3.55 (m, 2H), 2.44 (s, 3H), 2.09-2.03 (m, 1H), 1.96-1.91 (m, 1H), 1.38-1.32 (m, 5H), 1.24-1.19 (m, 3H), 0.88 (s, 9H).

### Synthesis of (R)-tert-butyl 4-(3-amino-4-(phenylthio)butyl)piperazine-1-carboxylate (Intermediate KK)

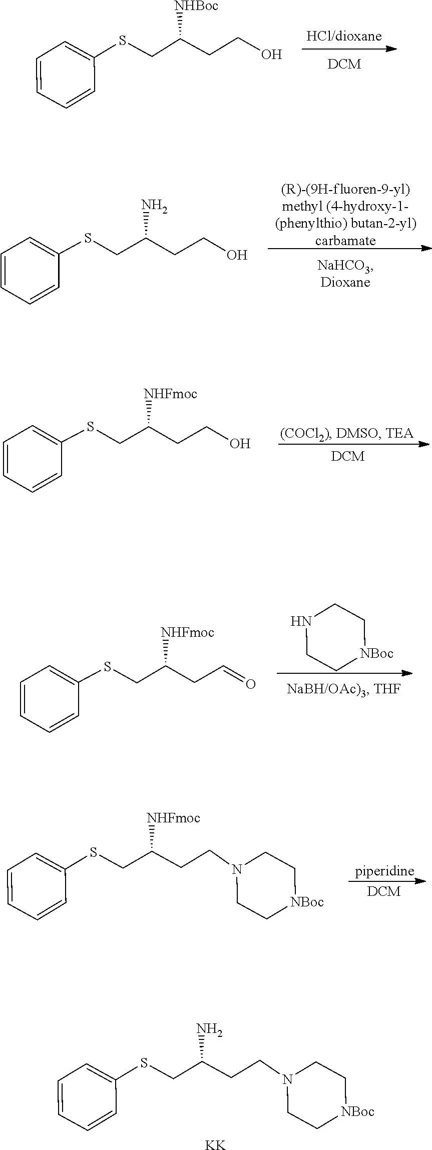

### Step 1—(R)-3-amino-4-(phenylthio)butan-1-ol

To a solution of tert-butyl N-[(1R)-3-hydroxy-1-(phenylsulfanylmethyl)propyl]carbamate (100 mg, 336 μmol, CAS #1240408-71-2) in DCM (1 mL) was added HCl/dioxane (4 M, 0.1 mL). The mixture was stirred at 25° C. for 16 hrs. On completion, the reaction mixture was concentrated under reduced pressure to remove solvent to give the title compound (50 mg, HCl salt) as a yellow solid. LC-MS (ESI⁺) m/z 181.1 (M+H)⁺.

### Step 2—(R)-(9H-fluoren-9-yl)methyl (4-hydroxy-1-(phenylthio)butan-2-yl)carbamate

To a solution of (R)-3-amino-4-(phenylthio)butan-1-ol (700 mg, 3 mmol, HCl) in dioxane (14 mL) was added (2,5-dioxopyrrolidin-1-yl) 9H-fluoren-9-ylmethyl carbonate (1.21 g, 3.59 mmol) and NaHCO₃ (755 mg, 8.98 mmol) at 0° C. The mixture was stirred at 25° C. for 16 hrs. On completion, the reaction mixture was quenched with H₂O (50 mL) at 25° C. and then diluted with EA (50 mL) and extracted with EA (30 mL×2). The combined organic layers were washed with aqueous NaCl (50 mL×2), dried over Na₂SO₄, filtered and concentrated under reduced pressure to give a residue. The residue was purified by column chromatography (SiO₂, Petroleum ether/Ethyl acetate=100/1 to 1/1) to give the title compound (1.2 g, 96% yield) as white solid. LC-MS (ESI⁺) m/z 420.2 (M+H)⁺.

### Step 3—(R)-(9H-fluoren-9-yl)methyl (4-oxo-1-(phenylthio)butan-2-yl)carbamate

To a solution of DMSO (447 mg, 5.72 mmol) in DCM (30 mL) was added dropwise (COCl)₂ (726 mg, 5.72 mmol) at −70° C. over 0.5 hour. After addition, the mixture was stirred at this temperature for 0.5 hour, and then (R)-(9H-fluoren-9-yl)methyl (4-hydroxy-1-(phenylthio)butan-2-yl)carbamate (1.2 g. 2.86 mmol) in DCM (5 mL) was added dropwise at −70° C. The resulting mixture was stirred at −70° C. for 1 hr and then TEA (1.45 g, 14.3 mmol) was added dropwise at 0° C. The resulting mixture was stirred at 0° C. for 2 hrs. On completion, the reaction mixture was quenched with ice-water 20 mL at 0° C., and then diluted with H₂O (20 mL) and extracted with DCM (20 mL×3). The combined organic layers dried over Na₂SO₄, filtered and concentrated under reduced pressure to give the title compound (1.4 g) as a white solid. LC-MS (ESI⁺) m/z 418.3 (M+H)⁺.

### Step 4—(R)-tert-butyl 4-(3-((((9H-fluoren-9-yl)methoxy) carbonyl)amino)-4-(phenylthio)butyl)piperazine-1-carboxylate

To a solution of 9H-fluoren-9-ylmethyl N-[(1R)-3-oxo-1-(phenylsulfanylmethyl)propyl]carbamate (1.3 g, 3.11 mmol) and tert-butyl piperazine-1-carboxylate (579 mg, 3.11 mmol, CAS #143238-38-4) in THF (20 mL) was added NaBH(OAc)₃ (725 mg, 3.42 mmol). The mixture was then stirred at 25° C. for 2 hrs. On completion, the reaction mixture was quenched with H₂O (10 mL) at 25° C., and then diluted with EA (20 mL) and extracted with EA (50 mL×2). The combined organic layers were dried over Na₂SO₄, filtered and concentrated under reduced pressure to give the title compound (2 g) as yellow solid. LC-MS (ESI⁺) m/z 588.0 (M+H)⁺.

### Step 5—(R)-tert-butyl 4-(3-amino-4-(phenylthio)butyl)piperazine-1-carboxylate

To a solution of (R)-tert-butyl 4-(3-((((9H-fluoren-9-yl)methoxy) carbonyl)amino)-4-(phenylthio)butyl)piperazine-1-carboxylate (900 mg, 1.53 mmol) in DMF (10 mL) was added piperidine (1.55 g, 18.2 mmol). The mixture was then stirred at 25° C. for 4 hrs. On completion, the reaction mixture was quenched with H₂O (5 mL) at 25° C. The mixture was purified by prep-HPLC (FA condition) to give the title compound (470 mg, 84% yield) as a white solid. LC-MS (ESI⁺) m/z 366.1 (M+H)⁺.

### (R)-tert-butyl 4-(4-(phenylthio)-3-((4-sulfamoyl-2-((trifluoromethyl)sulfonyl)phenyl)amino)butyl)piperazine-1-carboxylate (Intermediate KL)

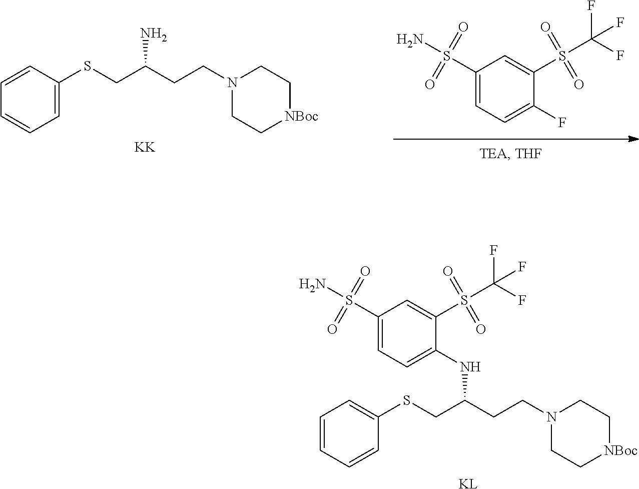

To a solution of (R)-tert-butyl 4-(3-amino-4-(phenylthio)butyl)piperazine-1-carboxylate (430 mg, 1.18 mmol, Intermediate KK) in DMF (10 mL) was added DIEA (760 mg, 5.88 mmol) and 4-fluoro-3-(trifluoromethylsulfonyl)benzenesulfonamide (433 mg, 1.41 mmol, CAS #1027345-08-9). The mixture was stirred at 25° C. for 16 hr. On completion, the reaction mixture was quenched with H₂O (10 mL) at 25° C. The mixture was purified by prep-HPLC (FA condition, 10% ACN-95% ACN, 30 min) to give the title compound (450 mg, 59% yield) as a white solid. LC-MS (ESI⁺) m/z 653.3 (M+H)⁺.

### Synthesis of (R)-ethyl 4-(4-((4′-chloro-[1,1′-biphenyl]-2-yl)(hydroxy)methyl)piperidin-1-yl)benzoate (Intermediate KM) and (S)-ethyl 4-(4-((4′-chloro-[1,1′-biphenyl]-2-yl)(hydroxy)methyl)piperidin-1-yl)benzoate (Intermediate KN)

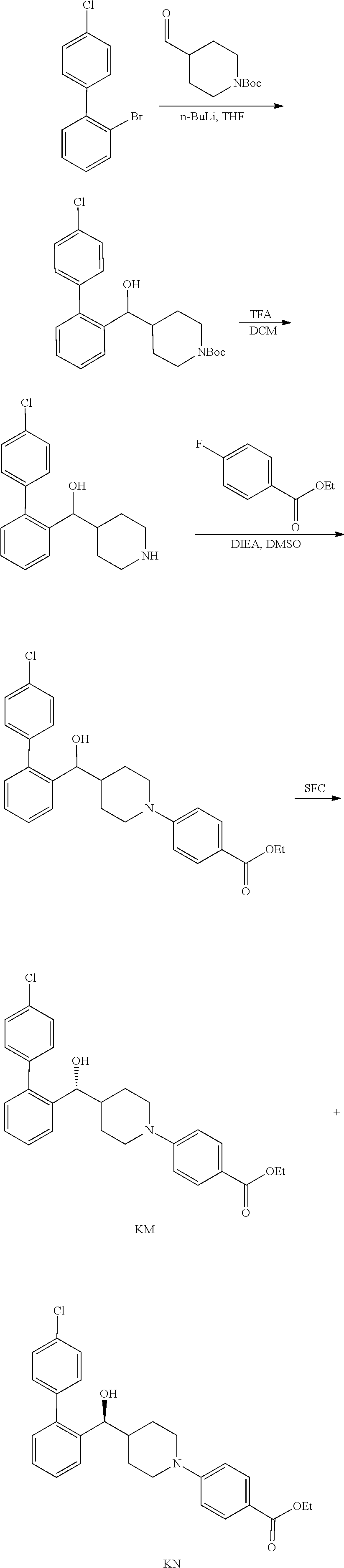

### Step 1—Tert-butyl 4-((4′-chloro-[1,1′-biphenyl]-2-yl)(hydroxy)methyl)piperidine-1-carboxylate

To a solution of 1-bromo-2-(4-chlorophenyl)benzene (5 g, 18.69 mmol, CAS #179526-95-5) in THF (100 mL) was added dropwise n-BuLi (2.5 M, 7.48 mL) at −78° C. After addition, the mixture was stirred at this temperature for 1 hr, and then tert-butyl 4-formylpiperidine-1-carboxylate (4.38 g, 20.6 mmol, CAS #137076-22-3) was added dropwise at −78° C. The resulting mixture was then stirred at −78° C. for 1 hr. On completion, the reaction mixture was quenched with NH₄Cl (50 mL) at −10° C., and then diluted with H₂O (50 mL) and extracted with EA (100 mL). The combined organic layers were washed with brine (45 mL), dried over Na₂SO₄, filtered and concentrated under reduced pressure to give a residue. The residue was purified by column chromatography (SiO₂, Petroleum ether/Ethyl acetate=100/1 to 3/1) to give the title compound (6.8 g, 72% yield) as white solid. LC-MS (ESI⁺) m/z 328.1 (M-56+H+16)⁺.

### Step 2—(4′-Chloro-[1,1′-biphenyl]-2-yl)(piperidin-4-yl) methanol

To a solution of tert-butyl 4-[[2-(4-chlorophenyl)phenyl]-hydroxy-methyl]piperidine-1-carboxylate (6 g, 20 mmol) in DCM (50 mL) and MeOH (2 mL) was added TFA (25.0 mL). The mixture was stirred at 25° C. for 2 hrs. On completion, the reaction mixture was concentrated under reduced pressure to remove the solvent. The residue was then diluted with EA (20 mL), washed with aq. NaHCO₃ (20 mL×2), dried over Na₂SO₄, filtered and concentrated under reduced pressure to give the title compound (5 g) as a white solid. LC-MS (ESI⁺) m/z 302.1 (M+H)⁺.

### Step 3—Ethyl 4-(4-((4′-chloro-[1,1′-biphenyl]-2-yl)(hydroxy)methyl)piperidin-1-yl)benzoate

To a solution of [2-(4-chlorophenyl)phenyl]-(4-piperidyl) methanol (770 mg, 2.55 mmol) and ethyl 4-fluorobenzoate (858 mg, 5.10 mmol, CAS #451-46-7) in DMSO (20 mL) was added DIEA (1.65 g, 12.8 mmol) and the mixture was stirred at 120° C. for 24 hrs. Next, TEA (1.29 g, 12.7 mmol) was added and the mixture was stirred at 120° C. for 12 hrs. On completion, the reaction mixture was quenched with aq. NH₄Cl (10 mL) at 0° C., and then diluted with EA (20 mL) and extracted with EA (30 mL×2). The combined organic layers were washed with aqueous NaCl (30 mL×2), dried over Na₂SO₄, filtered and concentrated under reduced pressure to give a residue. The residue was purified by column chromatography (SiO₂, Petroleum ether/Ethyl acetate=10/1 to 1/1) to give the title compound (2 g) as yellow solid. LC-MS (ESI⁺) m/z 450.2 (M+H)⁺.

### Step 4—(R)-ethyl 4-(4-((4′-chloro-[1,1′-biphenyl]-2-yl)(hydroxy)methyl)piperidin-1-yl)benzoate and (S)-ethyl 4-(4-((4′-chloro-[1,1′-biphenyl]-2-yl)(hydroxy)methyl)piperidin-1-yl)benzoate

Ethyl 4-(4-((4′-chloro-[1,1′-biphenyl]-2-yl)(hydroxy)methyl)piperidin-1-yl)benzoate was separated by SFC (condition: Column:Chiralpak IG-3 50×4.6 mm I.D., 3 um; Mobile phase: Phase A for CO2, and Phase B for IPA+ACN (0.05% DEA); Isocratic elution: IPA+ACN (0.05% DEA) Flow rate: 3 mL/min; Detector: PDA; Column Temp: 35C; Back Pressure: 100 Bar) to give (R)-ethyl 4-(4-((4′-chloro-[1,1′-biphenyl]-2-yl)(hydroxy)methyl)piperidin-1-yl)benzoate (270 mg, 40% yield) as a yellow solid (¹H NMR (400 MHz, CDCl₃-d) δ=7.89 (d, J=9.0 Hz, 2H), 7.61 (dd, J=1.0, 8.0 Hz, 1H), 7.46 (dt, J=1.4, 7.6 Hz, 1H), 7.42-7.33 (m, 3H), 7.25 (br s, 3H), 6.88-6.74 (m, 2H), 4.51 (dd, J=2.4, 8.0 Hz, 1H), 4.32 (q, J=7.0 Hz, 2H), 3.87 (br d, J=13.0 Hz, 1H), 3.69 (br d, J=12.0 Hz, 1H), 2.84-2.56 (m, 2H), 2.16-2.02 (m, 1H), 1.91-1.72 (m, 2H), 1.40-1.30 (m, 4H), 1.10 (br s, 2H) and (S)-ethyl 4-(4-((4′-chloro-[1,1′-biphenyl]-2-yl)(hydroxy)methyl)piperidin-1-yl)benzoate (290 mg, 45% yield) as a yellow solid (¹H NMR (400 MHz, CDCl₃-d) δ=7.89 (d, J=9.0 Hz, 2H), 7.64-7.58 (m, 1H), 7.46 (dt, J=1.2, 7.6 Hz, 1H), 7.41-7.33 (m, 3H), 7.25-7.19 (m, 3H), 6.88-6.73 (m, 2H), 4.51 (br d, J=8.2 Hz, 1H), 4.32 (q, J=7.2 Hz, 2H), 3.92-3.60 (m, 2H), 2.84-2.57 (m, 2H), 2.19-2.06 (m, 1H), 1.91-1.70 (m, 2H), 1.36 (t, J=7.2 Hz, 3H), 1.23-1.02 (m, 2H)). LC-MS (ESI⁺) m/z 450.4 (M+H)⁺ for both enantiomers. Absolute stereochemistry of the enantiomers was assigned arbitrarily.

### Synthesis of (S)-4-(4-((4′-chloro-[1,1′-biphenyl]-2-yl)(hydroxy)methyl)piperidin-1-yl)benzoic Acid (Intermediate KO)

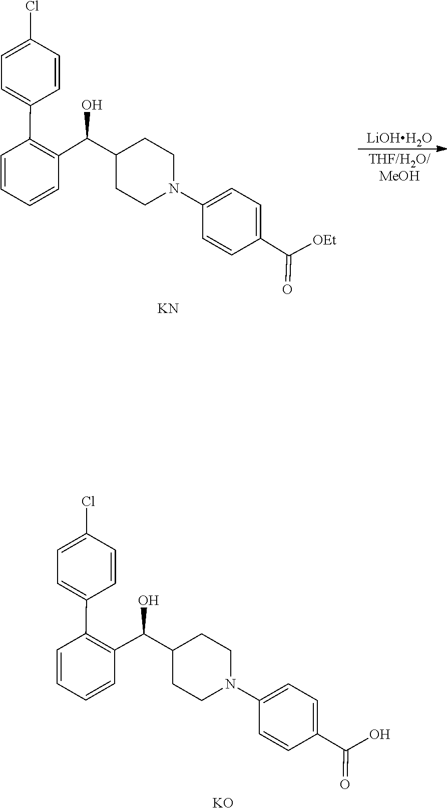 [SMILES: CCOC(=O)c1ccc(N2CCC([C@H](O)c3ccccc3-c3ccc(Cl)cc3)CC2)cc1 ; "LiOH·H2O" ; "KN" ; O=C(O)c1ccc(N2CCC([C@H](O)c3ccccc3-c3ccc(Cl)cc3)CC2)cc1 ; "KO"]

To a solution of ethyl 4-[4-[(S)-[2-(4-chlorophenyl)phenyl]-hydroxy-methyl]-1-piperidyl]benzoate (270 mg, 600 μmol, Intermediate KN) in THF (4 mL) and MeOH (1 mL) and H₂O (1 mL) was added LiOH·H₂O (251 mg, 6.00 mmol). The mixture was stirred at 50° C. for 16 hrs. On completion, the reaction mixture was concentrated under reduced pressure to remove THF and MeOH. The residue was diluted with H₂O (20 mL) and pH was adjusted to 2-3 by HCl (1 M), and extracted with EA (50 mL×2). The combined organic layers were washed with aqueous NaCl (40 mL×2), dried over Na₂SO₄, filtered and concentrated under reduced pressure to give the title compound (300 mg) as yellow oil. LC-MS (ESI⁺) m/z 422.0 (M+H)⁺.

### 4-(4-((S)-(4′-chloro-[1,1′-biphenyl]-2-yl)(hydroxy)methyl) piperidin-1-yl)-N-((4-(((R)-1-(phenylthio)-4-(piperazin-1-yl)butan-2-yl)amino)-3-((trifluoromethyl)sulfonyl)phenyl)sulfonyl)benzamide (Intermediate KP)

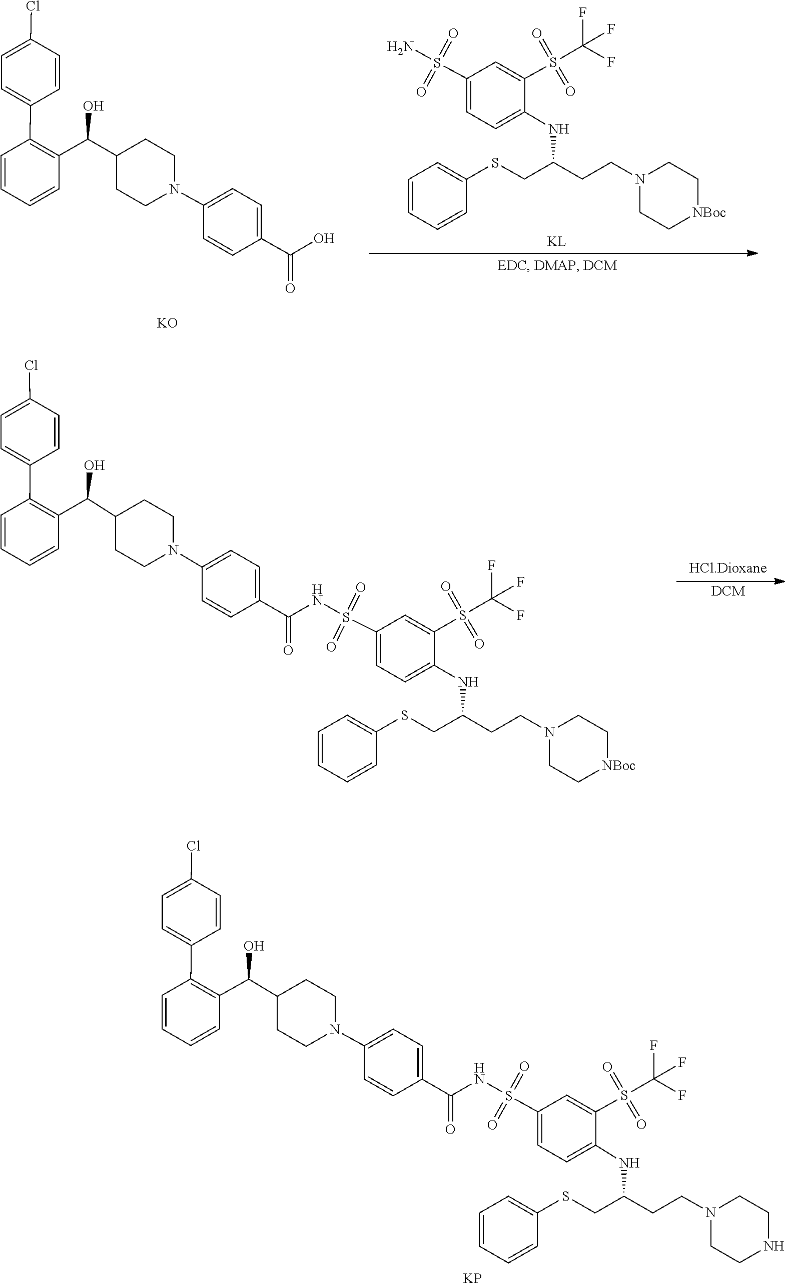

### Step 1—Tert-butyl 4-((R)-3-((4-(N-(4-(4-((S)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)piperazine-1-carboxylate

To a solution of 4-[4-[(S)-[2-(4-chlorophenyl)phenyl]-hydroxy-methyl]-1-piperidyl]benzoic acid (200 mg, 474 μmol, Intermediate KO) in dry DCM (5 mL) was added EDC (147 mg, 948 μmol) and tert-butyl 4-[(3R)-4-phenylsulfanyl-3-[4-sulfamoyl-2-(trifluoromethylsulfonyl)anilino]butyl]piperazine-1-carboxylate (309 mg, 474 μmol, Intermediate KL). Next, DMAP (145 mg, 1.19 mmol) was added, and the mixture was stirred at 25° C. for 16 hrs. On completion, the reaction mixture was quenched with H₂O (2 mL) at 25° C. The mixture was purified by prep-HPLC (FA condition) to give the title compound (350 mg, 70% yield) as white solid. LC-MS (ESI⁺) m/z 1056.6 (M+H)⁺.

### Step 2—4-(4-((S)-(4′-chloro-[1,1′-biphenyl]-2-yl)(hydroxy)methyl)piperidin-1-yl)-N-((4-(((R)-1-(phenylthio)-4-(piperazin-1-yl)butan-2-yl)amino)-3-((trifluoromethyl)sulfonyl)phenyl)sulfonyl)benzamide

To a solution of tert-butyl 4-[(3R)-3-[4-[[4-[4-[(S)-[2-(4-chlorophenyl)phenyl]-hydroxy-methyl]-1-piperidyl]benzoyl]sulfamoyl]-2-(trifluoromethylsulfonyl)anilino]-4-phenylsulfanyl-butyl]piperazine-1-carboxylate (350 mg, 331 μmol) in DCM (5 mL) was added HCl/dioxane (4 M, 1 mL). The mixture was then stirred at 25° C. for 2 hrs. On completion, the reaction mixture was concentrated under reduced pressure to give the title compound (350 mg, HCl) as white solid. LC-MS (ESI⁺) m/z 956.2 (M+H)⁺.

### Synthesis of 7-(((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)amino)-7-oxoheptanoic Acid (Intermediate KQ)

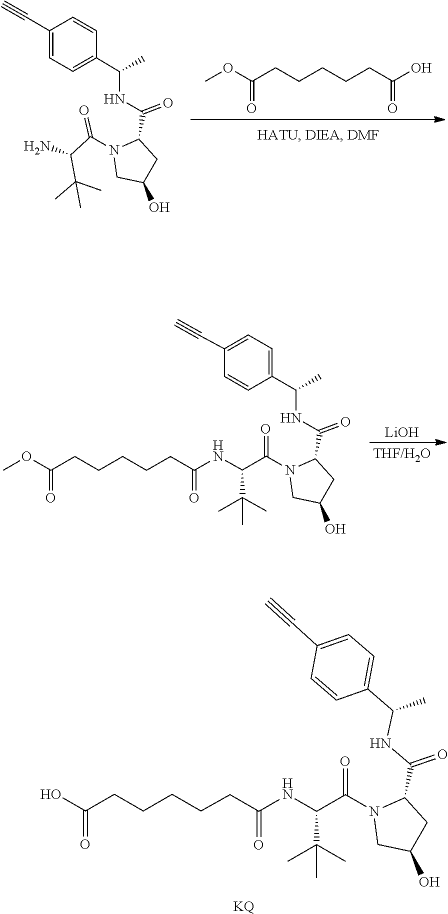

### Step 1—Ethyl 7-(((S)-1-((2S,4R)-2-(((S)-1-(4-ethynylphenyl)ethyl)carbamoyl)-4-hydroxypyrrolidin-1-yl)-3,3-dimethyl-1-oxobutan-2-yl)amino)-7-oxoheptanoate

To a solution of (2S,4R)-1-[(2S)-2-amino-3,3-dimethyl-butanoyl]-N-[(1S)-1-(4-ethynylphenyl)ethyl]-4-hydroxy-pyrrolidine-2-carboxamide (900 mg, 2.42 mmol, synthesized via Steps 1-2 of Intermediate BM) in DMF (20 mL) was added DIEA (939 mg, 7.27 mmol, 1.27 mL) and HATU (1.38 g, 3.63 mmol). Next, 7-ethoxy-7-oxo-heptanoic acid (547 mg, 2.91 mmol, CAS #33018-91-6) was added and the mixture was stirred at 25° C. for 3 hrs. On completion, the reaction mixture was diluted with H₂O (30 mL) and extracted with EA (20 mL×3). The combined organic layer was dried over Na₂SO₄, filtered and concentrated under reduced pressure to give the title compound (1.4 g) as a white solid. LC-MS (ESI⁺) m/z 542.3 (M+H)⁺.

### Step 2—7-(((S)-1-((2S,4R)-2-[[(S)-1-(4-ethynylphenyl)ethyl)carbamoyl)-4-hydroxypyrrolidin-1-yl)-3,3-dimethyl-1-oxobutan-2-yl)amino)-7-oxoheptanoic Acid

To a solution of ethyl 7-[[[(1S)-1-[(2S,4R)-2-[[(1S)-1-(4-ethynylphenyl)ethyl]carbamoyl]-4-hydroxy-pyrrolidine-1-carbonyl]-2,2-dimethyl-propyl]amino]-7-oxo-heptanoate (1.3 g, 2.40 mmol) in THF (20 mL) and H₂O (2 mL) was added LiOH (287 mg, 12.0 mmol). The mixture was stirred at 20° C. for 16 hrs. On completion, the reaction mixture was quenched with H₂O (10 mL) then hydrochloric acid (5 mL, 1 mol/L). A solid precipitated which was then filtered and dried under reduced pressure to give the crude product. The crude product was purified by reversed-phase HPLC (0.1% FA condition) to give the title compound (0.9 g) as a white solid. LC-MS (ESI⁺) m/z 514.4 (M+H)⁺.

### Synthesis of (R)-4-(4-((4′-chloro-[1,1′-biphenyl]-2-yl)(hydroxy)methyl)piperidin-1-yl)benzoic Acid (Intermediate KR)

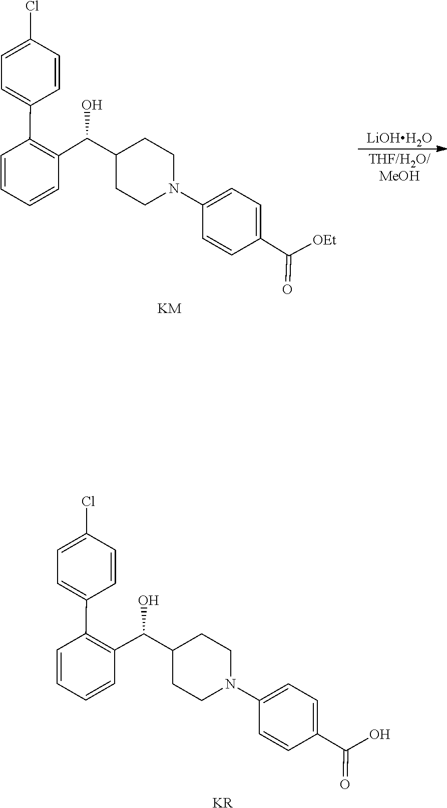

To a solution of ethyl 4-[4-[(R)-[2-(4-chlorophenyl)phenyl]-hydroxy-methyl]-1-piperidyl]benzoate (270 mg, 600 μmol, Intermediate KM) in THF (4 mL), MeOH (1 mL) and H₂O (1 mL) was added LiOH·H₂O (251 mg, 6.00 mmol). The mixture was stirred at 50° C. for 5 hrs. On completion, the reaction mixture was concentrated under reduced pressure to remove THF and MeOH. The residue was diluted with H₂O (20 mL) and the pH was adjusted to 2-3 with HCl (1 M), then extracted with EA (50 mL×2). The combined organic layers were washed with aqueous NaCl (40 mL×2), dried over Na₂SO₄, filtered and concentrated under reduced pressure to give a residue (290 mg) as a yellow solid. LC-MS (ESI⁺) m/z 422.0 (M+H)⁺.

### 4-(4-((R)-(4′-chloro-[1,1′-biphenyl]-2-yl)(hydroxy)methyl)piperidin-1-yl)-N-((4-(((R)-1-(phenylthio)-4-(piperazin-1-yl)butan-2-yl)amino)-3-((trifluoromethyl)sulfonyl)phenyl)sulfonyl)benzamide (Intermediate KS)

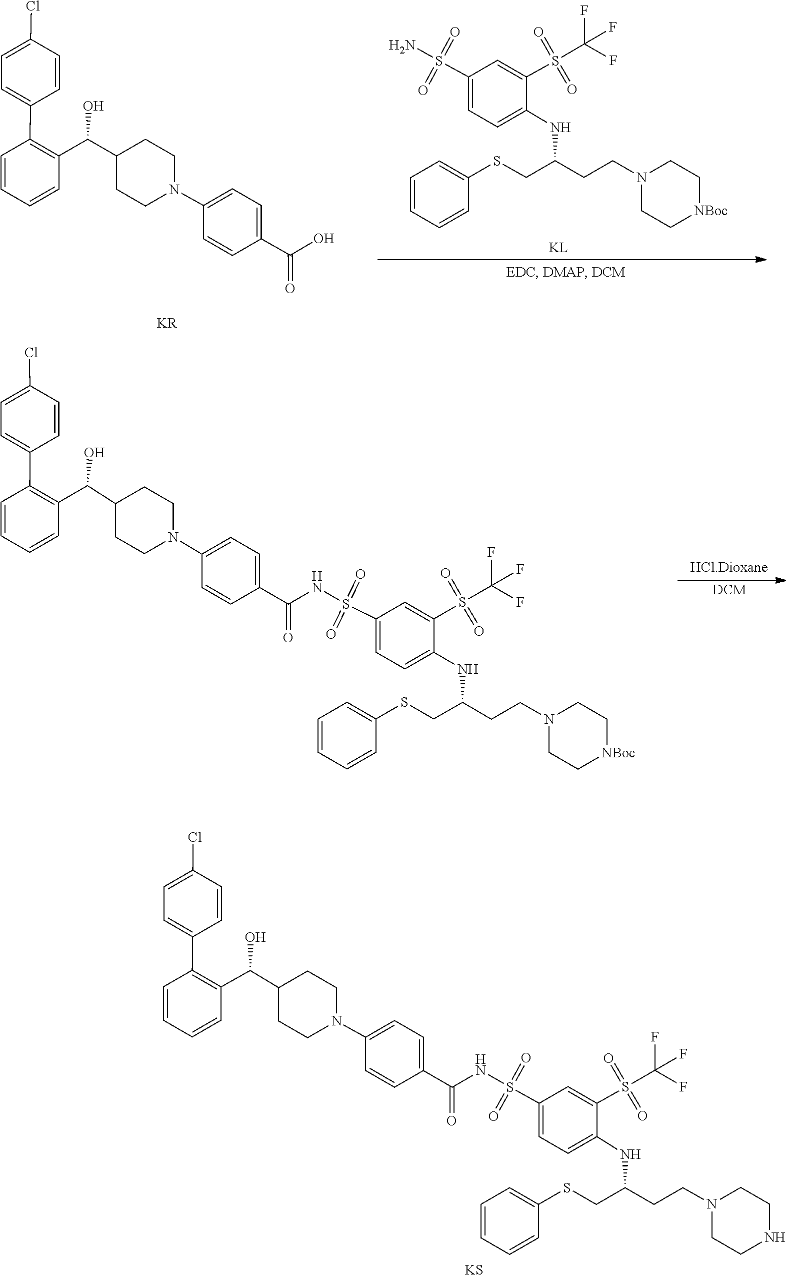

### Step 1—Tert-butyl 4-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)piperazine-1-carboxylate

To a solution of (R)-4-(4-((4′-chloro-[1,1′-biphenyl]-2-yl)(hydroxy)methyl)piperidin-1-yl)benzoic acid (280 mg, 664 μmol, Intermediate KR) and tert-butyl 4-[(3R)-4-phenylsulfanyl-3-[4-sulfamoyl-2-(trifluoromethylsulfonyl)anilino]butyl]piperazine-1-carboxylate (433 mg, 663 μmol, Intermediate KL) in DCM (2 mL) was added EDC (206 mg, 1.33 mmol) and DMAP (203 mg, 1.66 mmol). The mixture was then stirred at 25° C. for 16 hrs. On completion, the reaction mixture was concentrated under reduced pressure to give the title compound (270 mg, 39% yield) as yellow solid. LC-MS (ESI⁺) m/z 1056.5 (M+H)⁺.

### Step 2—4-(4-((R)-(4′-chloro-[1,1′-biphenyl]-2-yl)(hydroxy)methyl)piperidin-1-yl)-N-((4-(((R)-1-(phenylthio)-4-(piperazin-1-yl)butan-2-yl)amino)-3-((trifluoromethyl)sulfonyl)phenyl)sulfonyl)benzamide

To a solution of tert-butyl 4-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)piperazine-1-carboxylate (250 mg, 237 μmol) in DCM (5 mL) was added HCl/dioxane (4 M, 60 uL). The mixture was then stirred at 25° C. for 2 hrs. On completion, the reaction mixture was concentrated under reduced pressure to give the title compound (250 mg. HCl salt) as a white solid. LC-MS (ESI⁺) m/z 956.3 (M+H)⁺.

### Synthesis of 7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoic Acid (Intermediate KT)

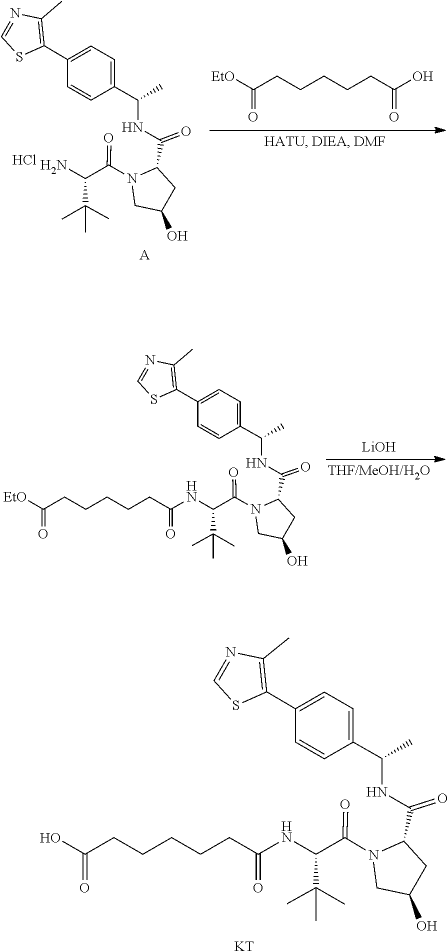

### Step 1—Ethyl 7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoate

To a solution of 7-ethoxy-7-oxo-heptanoic acid (635 mg, 3.37 mmol, CAS #33018-91-6) in DMF (20 mL) was added HATU (1.41 g, 3.71 mmol), DIEA (4.36 g, 33.7 mmol) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide hydrochloride (1.5 g, 3.37 mmol, Intermediate A). Then the reaction was stirred at 0° C. for 10 min. On completion, the reaction mixture was quenched with H₂O (20 mL) at 25° C. and then extracted with EA (100 mL×2). The combined organic layers were washed with brine (100 mL×2), dried over Na₂SO₄, filtered and the filtrate was concentrated under reduced pressure to give a residue. The residue was purified by column chromatography (SiO₂, Petroleum ether/Ethyl acetate=1/0 to 10/1) to give the title compound (2.5 g) as a white solid. LC-MS (ESI⁺) m/z 615.3. (M+H)⁺.

### Step 2—7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoic Acid

To a solution of ethyl 7-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-7-oxo-heptanoate (500 mg, 813 μmol) in THF (2.5 mL), H₂O (0.5 mL) and MeOH (2.5 mL) was added LiOH·H₂O (171 mg, 4.07 mmol). Then the mixture was stirred at 25° C. for 2 hrs. On completion, the reaction mixture was quenched with H₂O (10 mL) at 25° C., and 1M HCl solution was added until the pH=7, then extracted with DCM (20 mL×3). The combined organic layers were washed with brine (50 mL×2), dried over Na₂SO₄, filtered and the filtrate was concentrated under reduced pressure to give the title compound (300 mg) as a white solid. LC-MS (ESI⁺) m/z 587.5. (M+H)⁺.

### Synthesis of Tert-butyl (R)-4-((4′-chloro-2-(hydroxy(1-(4-(methoxycarbonyl)phenyl)piperidin-4-yl)methyl)-[1,1′-biphenyl]-4-yl)methyl)piperazine-1-carboxylate (Intermediate KU) and tert-butyl (S)-4-((4′-chloro-2-(hydroxy(1-(4-(methoxycarbonyl)phenyl)piperidin-4-yl)methyl)-[1,1′-biphenyl]-4-yl)methyl)piperazine-1-carboxylate (Intermediate KV)

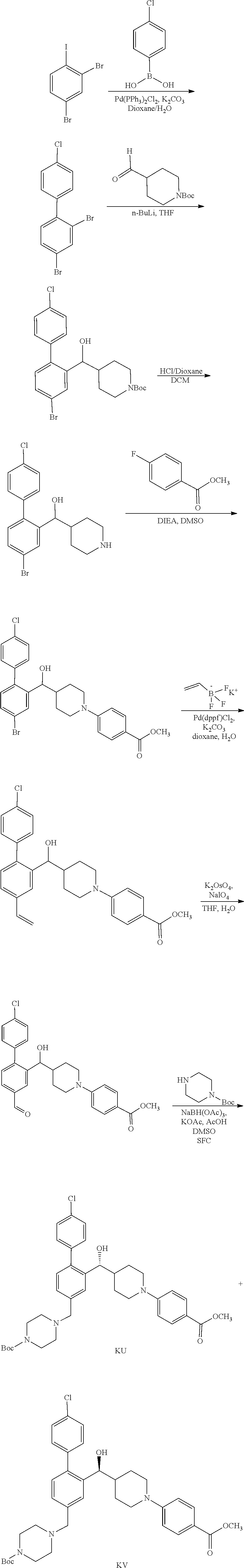

### Step 1—2,4-Dibromo-4′-chloro-1,1′-biphenyl

To a solution of 2,4-dibromo-1-iodobenzene (20.0 g, 55.2 mmol, CAS #19393-94-3), (4-chlorophenyl)boronic acid (12.1 g, 77.3 mmol, CAS #1679-18-1), Pd(PPh₃)₂Cl₂ (1.94 g, 2.76 mmol), and K₂CO₃ (19.1 g, 138 mmol) in H₂O (40 mL) and DME (160 mL) was degassed and purged with N₂ three times. Then the mixture was stirred at 80° C. for 12 hours under N₂ atmosphere. On completion, the mixture was concentrated under reduced pressure and purified by column chromatography (SiO₂, Petroleum ether/Ethyl acetate=1/0) to give the compound (17 g, 88% yield) as a white solid. LC-MS (ESI⁺) m/z 634.8 (M+H)⁺.

### Step 2—Tert-butyl 4-((4-bromo-4′-chloro-[1,1′-biphenyl]-2-yl)(hydroxy)methyl)piperidine-1-carboxylate

A solution of 2,4-dibromo-1-(4-chlorophenyl)benzene (10 g, 28.8 mmol) in THF (170 mL) was purged with N₂ for 3 times and cooled to −78° C., then n-butyllithium (2.5 M, 10.3 mL) was added and the mixture was stirred for 1 hr at −78° C. Then tert-butyl 4-formylpiperidine-1-carboxylate (6.77 g, 31.7 mmol, CAS #137076-22-3) was added and the mixture was stirred at −78° C. for 1 hr. On completion, the reaction mixture was quenched with NH₄Cl (200 mL) at 0° C., and then diluted with H₂O (30 mL) and extracted with EA (400 mL). The combined organic layer was dried over Na₂SO₄, filtered and concentrated under reduced pressure to give a residue. The residue was purified by prep-HPLC (FA condition) to give the title compound (8 g, 45% yield, FA) as a white solid. LC-MS (ESI⁺) m/z 407.9 (M+H)⁺.

### Step 3—(4-Bromo-4′-chloro-[1,1′-biphenyl]-2-yl)(piperidin-4-yl)methanol

To a solution of tert-butyl 4-((4-bromo-4′-chloro-[1,1′-biphenyl]-2-yl)(hydroxy)methyl)piperidine-1-carboxylate (8 g, 16.6 mmol) in DCM (200 mL) was added HCl/dioxane (4 M, 12.48 mL). The mixture was stirred at 25° C. for 2 hrs. On completion, the reaction mixture was concentrated under reduced pressure to give the title compound (6.9 g, HCl) as a white solid. LC-MS (ESI⁺) m/z 382.2 (M+H)⁺.

### Step 4—Ethyl 4-(4-((4-bromo-4′-chloro-[1,1′-biphenyl]-2-yl)(hydroxy)methyl)piperidin-1-yl)benzoate

A mixture of (4-bromo-4′-chloro-[1,1′-biphenyl]-2-yl)(piperidin-4-yl)methanol (6.9 g, 18 mmol), methyl 4-fluorobenzoate (4.19 g, 27.1 mmol, CAS #403-33-8), and DIEA (35.1 g, 271 mmol, 47.3 mL) in DMSO (120 mL) was degassed and purged with N₂ three times. Then the mixture was stirred at 120° C. for 12 hrs under N₂ atmosphere. On completion, the residue was diluted with H₂O (120 mL) and extracted with EA (120 mL×3). The combined organic layers were washed with aqueous NaCl (240 mL×3), dried over Na₂SO₄, filtered and concentrated under reduced pressure to give a residue. The residue was purified by column chromatography (SiO₂, Petroleum ether/Ethyl acetate=1/0 to 20/1) to give the title compound (4.5 g, 44% yield) as a white solid. LC-MS (ESI⁺) m/z 515.8 (M+H)⁺.

### Step 5—Ethyl 4-(4-((4′-chloro-4-vinyl-[1,1′-biphenyl]-2-yl)(hydroxy)methyl)piperidin-1-yl)benzoate

To a solution of methyl 4-(4-((4-bromo-4′-chloro-[1,1′-biphenyl]-2-yl)(hydroxy)methyl)piperidin-1-yl)benzoate (4.19 g, 8.13 mmol), potassium trifluoro (vinyl) borate (1.63 g, 12.2 mmol, CAS #13682-77-4), Pd(dppf)Cl₂ (594 mg, 813 μmol), and K₂CO₃ (3.37 g, 24.3 mmol) in dioxane (45 mL) and H₂O (7 mL) was degassed and purged with N₂ three times. Then the mixture was stirred at 80° C. for 5 hrs under N₂ atmosphere. The reaction mixture was concentrated under reduced pressure to give a residue. The residue was purified by column chromatography (SiO₂, Petroleum ether/Ethyl acetate=1/0 to 5/1) to give the title compound (2.8 g, 65% yield) as a yellow solid. LC-MS (ESI⁺) m/z 462.2 (M+H)⁺.

### Step 6—Ethyl 4-(4-((4′-chloro-4-formyl-[1,1′-biphenyl]-2-yl)(hydroxy)methyl)piperidin-1-yl)benzoate

To a solution of methyl 4-(4-((4′-chloro-4-vinyl-[1,1′-biphenyl]-2-yl)(hydroxy)methyl)piperidin-1-yl)benzoate (1.4 g, 3.03 mmol), K₂OsO₄·2H₂O (78.1 mg, 212 μmol), and NaIO₄ (3.24 g, 15.1 mmol, 839 uL) in THF (15 mL) and H₂O (3 mL) was degassed and purged with N₂ three times. Then the mixture was stirred at 25° C. for 1 hr under N₂ atmosphere. On completion, the reaction mixture was concentrated in vacuo to give the title compound (1.4 g) as a white solid. LC-MS (ESI⁺) m/z 464.0 (M+H)⁺.

### Step 7—Tert-butyl (R)-4-((4′-chloro-2-(hydroxy(1-(4-(methoxycarbonyl)phenyl)piperidin-4-yl)methyl)-[1,1′-biphenyl]-4-yl)methyl)piperazine-1-carboxylate and tert-butyl (S)-4-((4′-chloro-2-(hydroxy(1-(4-(methoxycarbonyl)phenyl)piperidin-4-yl)methyl)-[1,1′-biphenyl]-4-yl)methyl)piperazine-1-carboxylate

To a solution of tert-butyl piperazine-1-carboxylate (353 mg, 1.90 mmol, CAS #143238-38-4) in DMSO (10 mL) was added KOAc (253 mg, 2.59 mmol), AcOH (310 mg, 5.17 mmol, 295 uL) and methyl 4-(4-((4′-chloro-4-formyl-[1,1′-biphenyl]-2-yl)(hydroxy)methyl)piperidin-1-yl)benzoate (800 mg, 1.72 mmol) at 25° C. and the mixture was stirred for 1 hr. Then NaBH(OAc)₃ (913 mg, 4.31 mmol) was added at 0° C. and the mixture was stirred at 25° C. for 1 hr. On completion, the reaction mixture was concentrated under reduced pressure and purified by SFC (column: DAICEL CHIRALPAK AS (250 mm×30 mm, 10 um); mobile phase: [0.1% NH₃H₂O ETOH]; B %: 30%-30%, C6.7; 81 min) to give tert-butyl (R)-4-((4′-chloro-2-(hydroxy(1-(4-(methoxycarbonyl)phenyl)piperidin-4-yl)methyl)-[1,1′-biphenyl]-4-yl)methyl)piperazine-1-carboxylate (300 mg, 26% yield, 95% purity) as a yellow solid and tert-butyl (S)-4-((4′-chloro-2-(hydroxy(1-(4-(methoxycarbonyl)phenyl)piperidin-4-yl)methyl)-[1,1′-biphenyl]-4-yl)methyl)piperazine-1-carboxylate (300 mg, 25% yield). LC-MS (ESI⁺) m/z 634.2 (M+H)⁺. The absolute stereochemistry of the enantiomers was assigned arbitrarily.

### (S)-4-(4-((4-((4-(tert-butoxycarbonyl)piperazin-1-yl)methyl)-4′-chloro-[1,1′-biphenyl]-2-yl)(hydroxy)methyl)piperidin-1-yl)benzoic Acid (Intermediate KW)

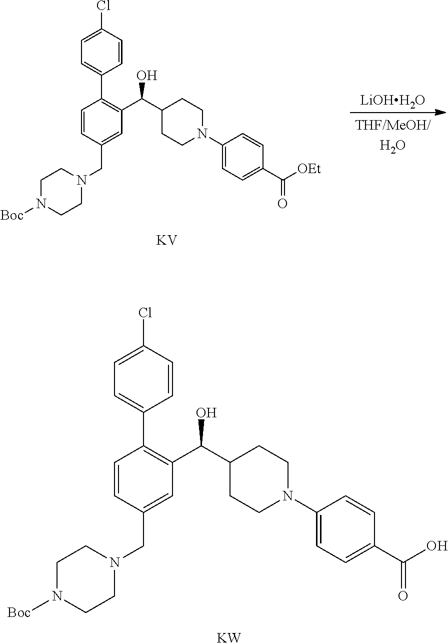 [SMILES: CCOC(=O)c1ccc(N2CCC([C@H](O)c3cc(CN4CCN(C(=O)OC(C)(C)C)CC4)ccc3-c3ccc(Cl)cc3)CC2)cc1 ; "LiOH·H2O" ; "KV" ; CC(C)(C)OC(=O)N1CCN(Cc2ccc(-c3ccc(Cl)cc3)c([C@@H](O)C3CCN(c4ccc(C(=O)O)cc4)CC3)c2)CC1 ; "KW"]

To a solution of tert-butyl (S)-4-((4′-chloro-2-((1-(4-(ethoxycarbonyl)phenyl)piperidin-4-yl)(hydroxy)methyl)-[1,1′-biphenyl]-4-yl)methyl)piperazine-1-carboxylate (300 mg, 473 μmol Intermediate KV) in THF (2 mL), H₂O (0.5 mL) and MeOH (0.5 mL) was added LiOH·H₂O (79.3 mg, 1.89 mmol). The mixture was then stirred at 50° C. for 12 hrs. On completion, HCl (1N) was added to the mixture until the pH=6, then the mixture was diluted with water (5 mL) and extracted with ethyl acetate/dichloromethane (5×3 mL). The combined organic layers were dried over anhydrous sodium sulfate, filtered and the filtrate was concentrated in vacuo to give the title compound (290 mg) as a white solid. LC-MS (ESI+) m/z 621.3 (M+H)⁺.

### Synthesis of (R)—N1-(2-((tert-butyldimethylsilyl)oxy)ethyl)-N1-methyl-4-(phenylthio)butane-1,3-diamine (Intermediate KX)

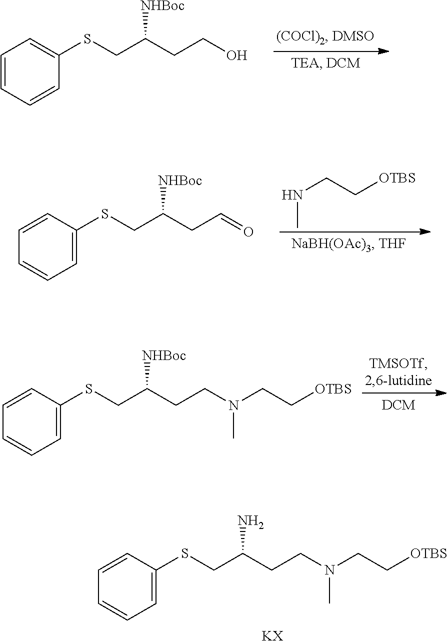

### Step 1—(R)-tert-butyl (4-oxo-1-(phenylthio)butan-2-yl)carbamate

To a solution of DMSO (525 mg, 6.72 mmol, CAS #1240408-71-2) in DCM (20 mL) was added dropwise oxalyl dichloride (853 mg, 6.72 mmol) at −70° C. over 30 min. After addition, the mixture was stirred at this temperature for 10 min, and then tert-butyl N-[(1R)-3-hydroxy-1-(phenylsulfanylmethyl)propyl]carbamate (1 g, 3.36 mmol) in DCM (5 mL) was added dropwise at −70° C. The resulting mixture was stirred at −70° C. for 1 hr and then TEA (1.70 g, 16.8 mmol) was added dropwise at 0° C. The resulting mixture was stirred at 0° C. for 20 min. On completion, the reaction mixture was quenched by addition of ice-water (20 mL) at 0° C., and then diluted with H₂O (20 mL) and extracted with DCM (20 mL×3). The combined organic layers dried over Na₂SO₄, filtered and the filtrate was concentrated under reduced pressure to give a residue. The residue was purified by column chromatography (SiO₂, Petroleum ether/Ethyl acetate=10/1 to 3/1) to give the title compound (730 mg) as a white solid. ¹H NMR (400 MHz, CHLOROFORM-d) δ=9.69 (s, 1H), 7.43-7.38 (m, 2H), 7.31 (t, J=7.6 Hz, 2H), 7.23 (d, J=7.4 Hz, 1H), 5.09-4.85 (m, 1H), 4.32-4.05 (m, 1H), 3.31-3.00 (m, 2H), 2.88-2.75 (m, 2H), 1.43 (s, 10H).

### Step 2—(R)-tert-butyl (4-((2-((tert-butyldimethylsilyl)oxy)ethyl)(methyl)amino)-1-(phenylthio)butan-2-yl)carbamate

To a solution of (R)-tert-butyl (4-oxo-1-(phenylthio)butan-2-yl)carbamate and 2-[tert-butyl(dimethyl)silyl]oxy-N-methyl-ethanamine (444 mg, 2.35 mmol, CAS #204580-28-9) in THF (2 mL) was added NaBH(OAc)₃ (452 mg, 2.13 mmol). The mixture was stirred at 25° C. for 2 hrs. On completion, the reaction mixture was quenched with H₂O (20 mL) at 0° C. and then diluted with H₂O (10 mL) and extracted with EA (30 mL×2). The combined organic layer was dried over Na₂SO₄, filtered and the filtrate was concentrated under reduced pressure to give the tittle compound (1.1 g) as a brown solid. LC-MS (ESI+) m/z 469.5 (M+H)⁺.

### Step 3—(R)—N1-(2-((tert-butyldimethylsilyl)oxy)ethyl)-N1-methyl-4-(phenylthio)butane-1,3-diamine

To a solution of (R)-tert-butyl (4-((2-((tert-butyldimethylsilyl)oxy)ethyl)(methyl)amino)-1-(phenylthio)butan-2-yl)carbamate (1 g, 2.13 mmol) in DCM (10 mL) was added trimethylsilyl trifluoromethanesulfonate (2.37 g, 10.7 mmol) and 2,6-dimethylpyridine (3.43 g, 32.0 mmol) at 0° C. The mixture was then stirred at 25° C. for 24 hrs. On completion, the reaction mixture was quenched with H₂O (30 mL) at 25° C., and extracted with DCM (50 mL). The combined organic layer was washed with H₂O (30 mL×2), dried over Na₂SO₄, filtered and the filtrate was concentrated under reduced pressure to give the title compound (3 g) as a white solid. LC-MS (ESI+) m/z 369.1 (M+H)⁺.

### Synthesis of (R)-4-((4-((2-((tert-butyldimethylsilyl)oxy)ethyl)(methyl)amino)-1-(phenylthio)butan-2-yl)amino)-3-((trifluoromethyl)sulfonyl)benzenesulfonamide (Intermediate KY)

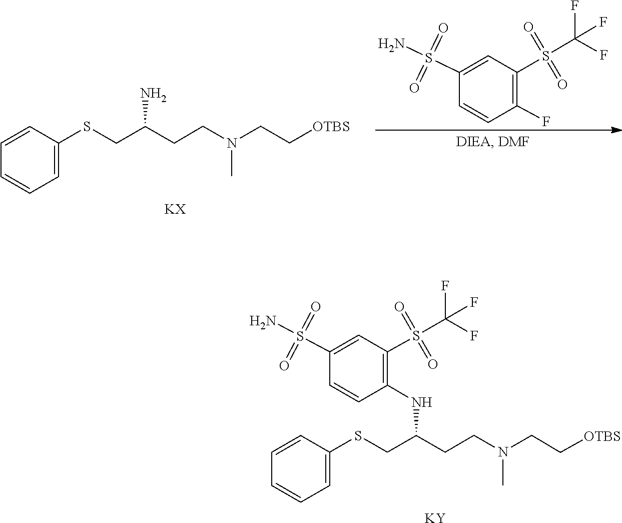

To a solution of (R)—N1-(2-((tert-butyldimethylsilyl)oxy)ethyl)-N1-methyl-4-(phenylthio)butane-1,3-diamine (2.96 g, 8.04 mmol, Intermediate KX) in DMF (30 mL) was added DIEA (2.73 g, 21.2 mmol) and 4-fluoro-3-(trifluoromethylsulfonyl)benzenesulfonamide (1.3 g, 4.23 mmol, CAS #1027345-08-9). The mixture was then stirred at 25° C. for 16 hrs. On completion, the reaction mixture was quenched with H₂O (10 mL) at 25° C. The residue was purified by prep-HPLC (FA condition, 10% ACN-95% ACN, 30 min) to give the title compound (600 mg, 22% yield) as a brown oil. LC-MS (ESI+) m/z 656.3 (M+H)⁺.

### 4-(4-((S)-(4′-chloro-4-(piperazin-1-ylmethyl)-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide (Intermediate KZ)

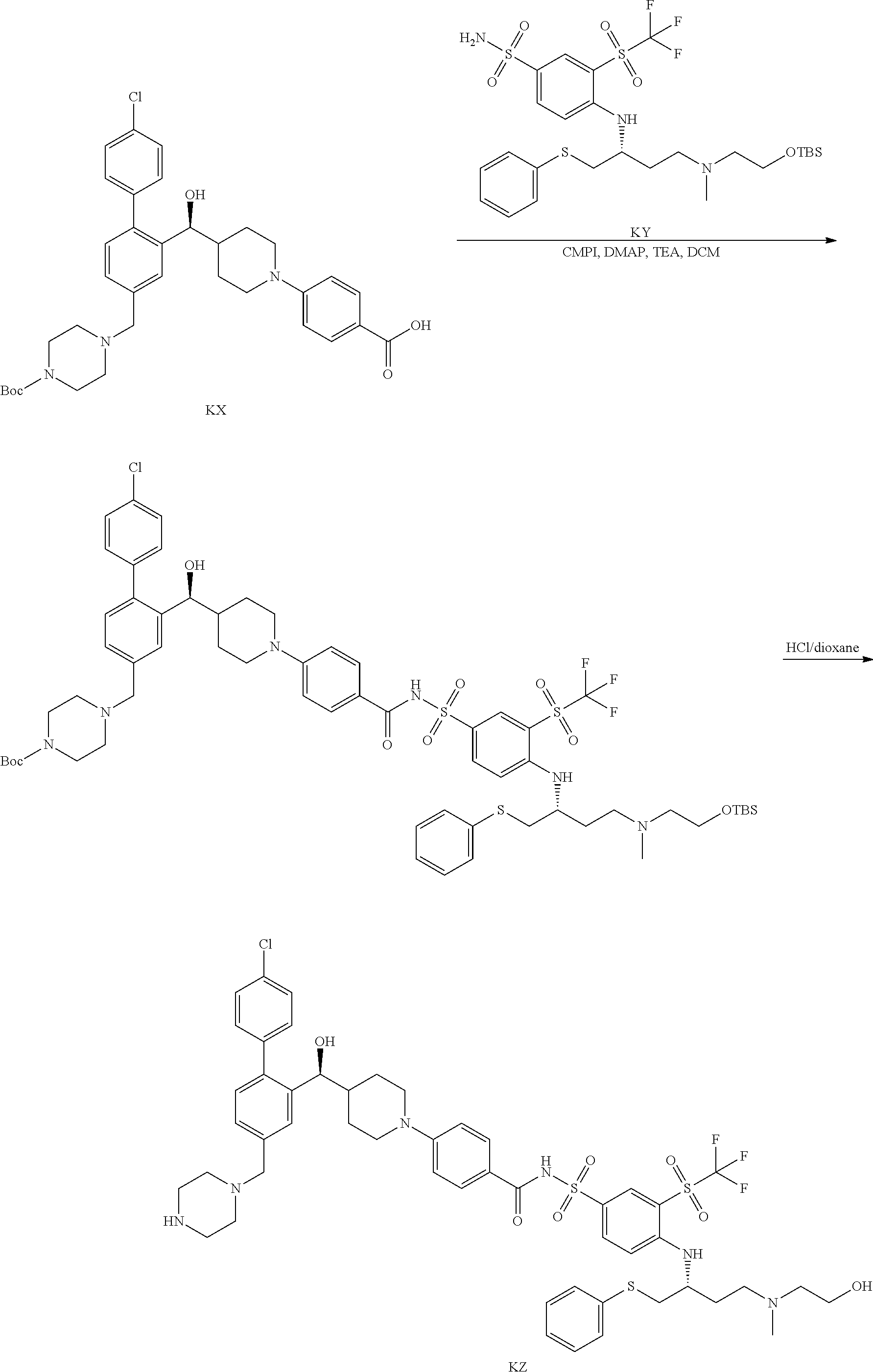

### Step 1—Tert-butyl 4-((2-((S)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)methyl)piperazine-1-carboxylate

To a solution of (S)-4-(4-((4-((4-(tert-butoxycarbonyl)piperazin-1-yl)methyl)-4′-chloro-[1,1′-biphenyl]-2-yl)(hydroxy)methyl)piperidin-1-yl)benzoic acid (50.0 mg, 80.6 μmol, Intermediate KW) in DCM (2 mL) was added 2-chloro-1-methyl-pyridin-1-ium iodide (30.9 mg, 120 μmol), TEA (24.4 mg, 241 μmol, 33.6 uL), DMAP (9.85 mg, 80.6 μmol) and (R)-4-((4-((2-((tert-butyldimethylsilyl)oxy)ethyl)(methyl)amino)-1-(phenylthio)butan-2-yl)amino)-3-((trifluoromethyl)sulfonyl)benzenesulfonamide (52.8 mg, 80.6 μmol, Intermediate KY). The mixture was then stirred at 25° C. for 2 hrs. On completion, the reaction mixture was concentrated under reduced pressure to give a residue. The residue was purified by prep-HPLC (0.1% FA condition) to give the title compound (80 mg, 57% yield, FA) as a white solid. LC-MS (ESI+) m/z 1259.4 (M+H)⁺.

### Step 2—4-(4-((S)-(4′-chloro-4-(piperazin-1-ylmethyl)-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide

To a solution of tert-butyl 4-((2-((S)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)methyl)piperazine-1-carboxylate (80.0 mg, 63.5 μmol) in DCM (2 mL) was added HCl/dioxane (4 M, 15.9 uL). The mixture was then stirred at 25° C. for 1 hr. On completion, the reaction mixture was concentrated under reduced pressure to give a residue. The residue was purified by prep-HPLC (column: Welch Xtimate C18 150×25 mm×5 um; mobile phase: [water (ammonia hydroxide v/v)-ACN]; B %: 20%-50%, 2 min) to give the title compound (55 mg, 83% yield) as an off-white solid. ¹H NMR (400 MHz, DMSO-d₆) δ=8.03 (d, J=2.0 Hz, 1H), 7.89 (dd, J=2.0, 9.2 Hz, 1H), 7.67 (d, J=8.8 Hz, 2H), 7.52 (s, 1H), 7.49 (d, J=8.4 Hz, 2H), 7.37-7.16 (m, 8H), 7.11 (d, J=7.6 Hz, 1H), 6.85-6.77 (m, 2H). 6.71 (d, J=9.2 Hz, 2H), 5.23 (d, J=4.4 Hz, 1H), 4.46-4.31 (m, 2H), 3.99 (d, J=5.6 Hz, 1H), 3.71 (d, J=12.0 Hz, 1H), 3.59 (s, 2H), 3.54 (s, 1H), 3.43-3.40 (m, 2H), 3.27 (d, J=5.6 Hz, 2H), 3.23-3.18 (m, 2H), 3.06 (s, 4H), 2.58-2.52 (m, 4H), 2.46-2.32 (m, 6H), 2.14 (s, 3H), 1.94-1.82 (m, 2H), 1.74-1.65 (m, 1H), 1.62-1.53 (m, 1H), 1.19-1.07 (m, 1H), 1.05-0.97 (m, 1H), 0.96-0.81 (m, 1H). LC-MS (ESI+) m/z 1045.3 (M+H)⁺.

### Synthesis of Tert-butyl 4-(3-((4-sulfamoyl-2-((trifluoromethyl)sulfonyl)phenyl)amino)propyl)piperazine-1-carboxylate (Intermediate LA)

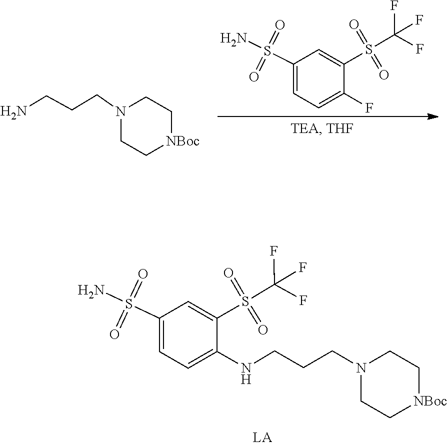 [SMILES: CC(C)(C)OC(=O)N1CCN(CCCN)CC1 ; NS(=O)(=O)c1ccc(F)c(S(=O)(=O)C(F)(F)F)c1 ; CC(C)(C)OC(=O)N1CCN(CCCNc2ccc(S(N)(=O)=O)cc2S(=O)(=O)C(F)(F)F)CC1]

To a solution of 4-fluoro-3-((trifluoromethyl)sulfonyl)benzenesulfonamide (1.26 g, 4.11 mmol, CAS #1027345-08-9) in THF (10 mL) was added tert-butyl 4-(3-aminopropyl)piperazine-1-carboxylate (1 g, 4.11 mmol, CAS #373608-48-1) and TEA (1.25 g, 12.3 mmol, 1.72 mL). The mixture was then stirred at 50° C. for 12 hrs. On completion, the mixture was diluted with H₂O (50 mL), and extracted with EtOAc (30 mL×3). The combined organic layers were washed with brine (20 mL×3), dried over anhydrous Na₂SO₄, filtered and the filtrate was concentrated to give the title compound (2.3 g) as a yellow oil. LC-MS (ESI⁺) m/z 531.2 (M+H)⁺.

### 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-(piperazin-1-yl)propyl)amino)-3-((trifluoromethyl)sulfonyl)phenyl)sulfonyl)benzamide (Intermediate LB)

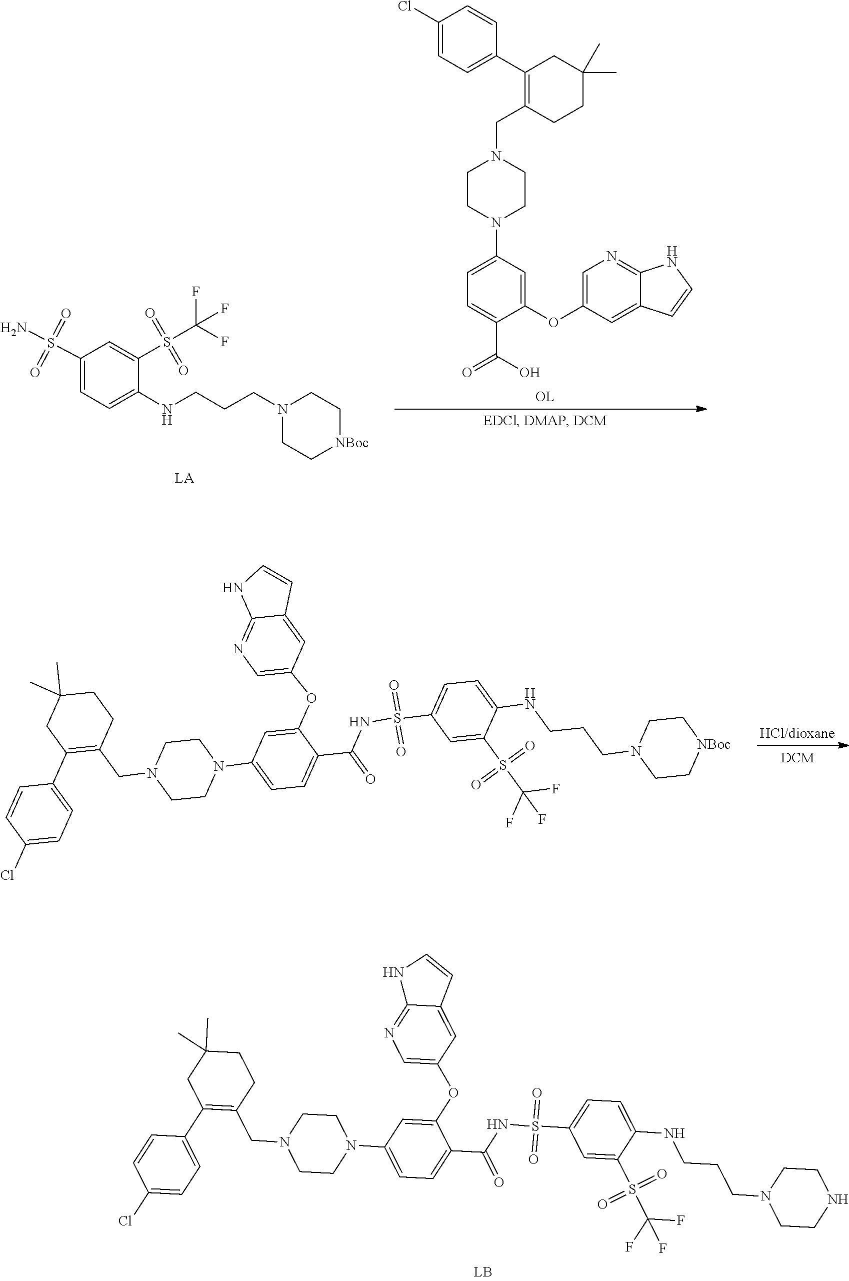 [SMILES: CC(C)(C)OC(=O)N1CCN(CCCNc2ccc(S(N)(=O)=O)cc2S(=O)(=O)C(F)(F)F)CC1 ; CC1(C)CCC(CN2CCN(c3ccc(C(=O)O)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(NCCCN5CCN(C(=O)OC(C)(C)C)CC5)c(S(=O)(=O)C(F)(F)F)c4)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(NCCCN5CCNCC5)c(S(=O)(=O)C(F)(F)F)c4)c(Oc4cnc5[nH]ccc5c4)c3)CC2)=C(c2ccc(Cl)cc2)C1]

### Step 1—Tert-butyl 4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazine-1-carboxylate

To a solution of tert-butyl 4-(3-((4-sulfamoyl-2-((trifluoromethyl)sulfonyl)phenyl)amino)propyl)piperazine-1-carboxylate (204 mg, 385 μmol, Intermediate LA) in DCM (4 mL) was added 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (200 mg, 350 μmol, Intermediate OL), DMAP (107 mg, 875 μmol) and EDC (109 mg, 700 μmol, 124 μL). Then the mixture was stirred at 30° C. for 6 hrs. On completion, the mixture was concentrated to give a crude product and then purified by reversed-phase HPLC (0.1% FA condition) to give the title compound (500 mg, 94% yield) as a gray solid. LC-MS (ESI⁺) m/z 542.5 (M/2+H)⁺.

### Step 2—2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-(piperazin-1-yl)propyl)amino)-3-(trifluoromethyl)sulfonyl)phenyl)sulfonyl)benzamide

To a solution of tert-butyl 4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazine-1-carboxylate (100 mg, 92.3 μmol) in DCM (4 mL) was added HCl/dioxane (4 M, 1 mL), then the mixture was stirred at 20° C. for 1 hr. On completion, the mixture was concentrated to give a crude product. The crude product was purified by reversed-phase HPLC (0.1% FA condition) to give the title compound (45 mg, 49% yield) as a white solid. ¹H NMR (400 MHz, DMSO-d₆) δ ppm 11.52 (br s, 1H), 8.18 (s, 1H), 7.97 (d, J=2.0 Hz, 1H), 7.92 (d, J=2.8 Hz, 1H), 7.74 (dd, J=9.2, 2.0 Hz, 1H), 7.57 (d, J=8.8 Hz, 1H), 7.42 (t, J=2.8 Hz, 1H), 7.32-7.36 (m, 2H), 7.27 (d, J=2.4 Hz, 1H), 7.03-7.07 (m, 2H), 6.96 (br t, J=5.2 Hz. 1H), 6.73 (d, J=9.2 Hz, 1H), 6.61 (dd, J=8.8, 2.4 Hz, 1H), 6.31 (dd, J=3.2, 2.0 Hz, 1H), 6.25 (d, J=2.0 Hz, 1H), 3.29 (br d, J=6.0 Hz, 4H), 3.03-3.06 (m, 4H), 3.00 (br s, 4H), 2.73 (s, 2H), 2.54 (br s, 2H), 2.38 (br t, J=6.4 Hz, 2H), 2.20 (br s, 4H), 2.16 (br s, 2H), 1.96 (br s, 2H), 1.68 (quin, J=6.4 Hz, 2H), 1.39 (br t, J=6.4 Hz, 2H), 0.93 (s, 6H). LC-MS (ESI⁺) m/z 983.6 (M+H)⁺.

### Synthesis of (R)-4-((4-(dimethylamino)-1-(phenylthio)butan-2-yl)amino)-3-nitrobenzenesulfonamide (Intermediate LC)

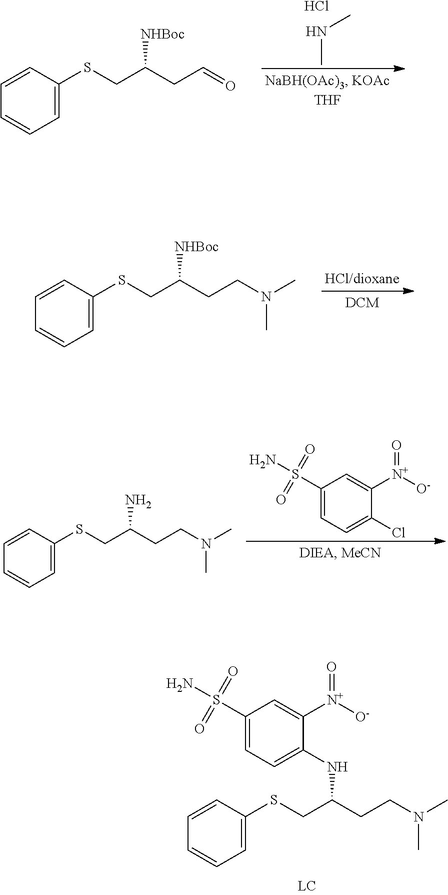

### Step 1—(R)-tert-butyl (4-(dimethylamino)-1-(phenylthio)butan-2-yl)carbamate

To a solution of (R)-tert-butyl (4-oxo-1-(phenylthio)butan-2-yl)carbamate (5 g, 16.9 mmol, synthesized via Step 1 of Intermediate KX) in THF (50 mL) was added dimethylamine hydrochloride (2.76 g, 33.9 mmol, 3.10 mL, HCl), NaBH(OAc)₃ (8.97 g, 42.3 mmol) and KOAc (2.49 g, 25.4 mmol). The mixture was then stirred at 20° C. for 1 hr. On completion, the mixture was diluted with H₂O (100 mL) and extracted with DCM (100 mL×3), dried over anhydrous Na₂SO₄ and concentrated to give the title compound (6 g) as light yellow oil. LC-MS (ESI⁺) m/z 325.2 (M+H)⁺.

### Step 2—(R)—N1,N1-dimethyl-4-(phenylthio)butane-1,3-diamine

To a solution of (R)-tert-butyl (4-(dimethylamino)-1-(phenylthio)butan-2-yl)carbamate (3.2 g, 9.86 mmol) in DCM (30 mL) was added HCl/EtOAc (4 M, 10 mL). The mixture was then stirred at 25° C. for 1 hr. On completion, the mixture was concentrated to give the title compound (3 g. HCl) as a yellow solid. LC-MS (ESI⁺) m/z 225.1 (M+H)⁺.

### Step 3—(R)-4-((4-(dimethylamino)-1-(phenylthio)butan-2-yl)amino)-3-nitrobenzenesulfonamide

To a solution of (R)—N1,N1-dimethyl-4-(phenylthio)butane-1,3-diamine (3.3 g, 12.7 mmol, HCl) in ACN (60 mL) was added DIEA (8.18 g, 63.3 mmol, 11.0 mL) and 4-chloro-3-nitrobenzenesulfonamide (3.59 g, 15.2 mmol, CAS #97-09-6). The mixture was stirred at 80° C. for 12 hrs. On completion, the mixture was concentrated to give the crude product. The crude product was then purified by reversed-phase HPLC (0.1% NH₄HCO₃ condition) to give the title compound (1.4 g) as a light yellow solid. LC-MS (ESI⁺) m/z 425.1 (M+H)⁺.

### Synthesis of Methyl 4-(4-((4-bromo-4′-chloro-[1,1′-biphenyl]-2-yl)methyl)piperazin-1-yl)benzoate (Intermediate LD)

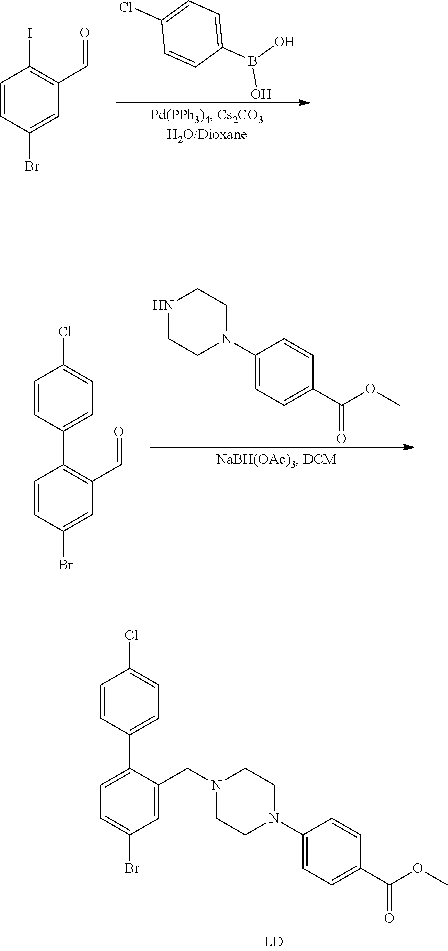 [SMILES: O=Cc1cc(Br)ccc1I ; OB(O)c1ccc(Cl)cc1 ; O=Cc1cc(Br)ccc1-c1ccc(Cl)cc1 ; COC(=O)c1ccc(N2CCNCC2)cc1 ; COC(=O)c1ccc(N2CCN(Cc3cc(Br)ccc3-c3ccc(Cl)cc3)CC2)cc1]

### Step 1—4-bromo-4′-chloro-[1,1′-biphenyl]-2-carbaldehyde

A mixture of 5-bromo-2-iodobenzaldehyde (4.8 g, 15.4 mmol, CAS #689291-89-2), (4-chlorophenyl)boronic acid (2.66 g, 16.9 mmol, CAS #1679-18-1), Pd(PPh₃)₄ (356 mg, 308 μmol), and Cs₂CO₃ (10.0 g, 30.8 mmol) in H₂O (12 mL) and dioxane (60 mL) was degassed and purged with N₂ three times. Then the mixture was stirred at 90° C. for 4 hours under N₂ atmosphere. On completion, the reaction mixture was diluted with water (50 mL) and extracted by ethyl acetate (2×50 mL). The combined organic layers were washed by brine (100 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 1/1) to give the title compound (3.9 g, 84% yield) as a white solid. LC-MS (ESI⁺) m/z 294.7 (M+H)⁺.

### Step 2—Methyl 4-(4-((4-bromo-4′-chloro-[1,1′-biphenyl]-2-yl)methyl)piperazin-1-yl)benzoate

To a solution of 4-bromo-4′-chloro-[1,1′-biphenyl]-2-carbaldehyde (3.9 g, 13.2 mmol) and methyl 4-(piperazin-1-yl)benzoate (2.64 g, 12.0 mmol, CAS #163210-97-7) in DCM (2 mL) was added NaBH(OAc)₃ (2.54 g, 12.0). The mixture was then stirred at 25° C. for 16 hrs. On completion, the reaction mixture was filtered and the filtrate was concentrated under reduced pressure to give a residue. The residue was purified by column chromatography (SiO₂, Petroleum ether/Ethyl acetate=1/0 to 1/1) to give the title compound (3.2 g, 53% yield) as a white solid. LC-MS (ESI⁺) m/z 501.1 (M+H)⁺.

### Synthesis of 4-(4-((4-(4-(tert-butoxycarbonyl)piperazin-1-yl)-4′-chloro-[1,1′-biphenyl]-2-yl)methyl)piperazin-1-yl)benzoic Acid (Intermediate LE)

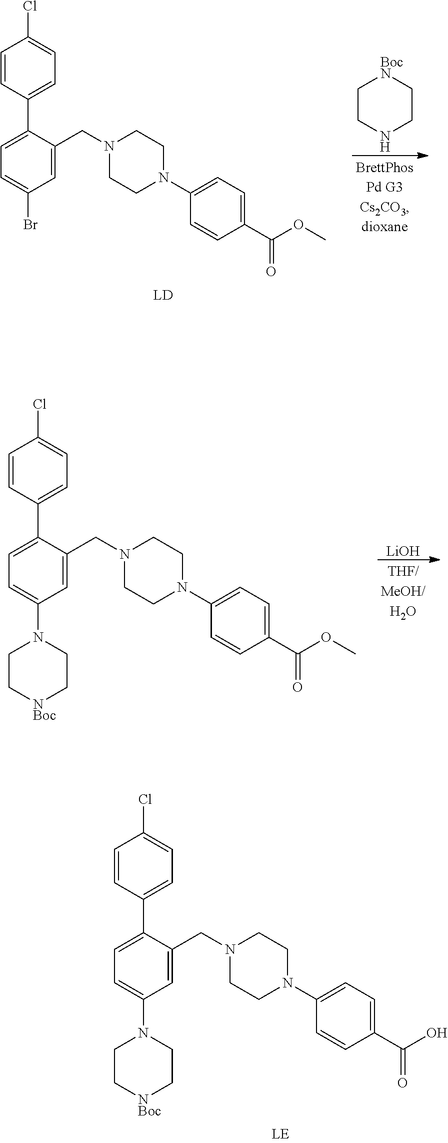 [SMILES: COC(=O)c1ccc(N2CCN(Cc3cc(Br)ccc3-c3ccc(Cl)cc3)CC2)cc1 ; CC(C)(C)OC(=O)N1CCNCC1 ; COC(=O)c1ccc(N2CCN(Cc3cc(N4CCN(C(=O)OC(C)(C)C)CC4)ccc3-c3ccc(Cl)cc3)CC2)cc1 ; "LiOH" ; CC(C)(C)OC(=O)N1CCN(c2ccc(-c3ccc(Cl)cc3)c(CN3CCN(c4ccc(C(=O)O)cc4)CC3)c2)CC1]

### Step 1—Tert-butyl 4-(4′-chloro-2-((4-(4-(methoxycarbonyl)phenyl)piperazin-1-yl)methyl)-[1,1′-biphenyl]-4-yl)piperazine-1-carboxylate

A mixture of methyl 4-(4-((4-bromo-4′-chloro-[1,1′-biphenyl]-2-yl)methyl)piperazin-1-yl)benzoate (500 mg, 1.00 mmol, Intermediate LD), tert-butyl piperazine-1-carboxylate (373 mg, 2.00 mmol, CAS #57260-71-6), BrettPhos Pd G3 (90.7 mg, 100 μmol), and Cs₂CO₃ (652 mg, 2.00 mmol) in dioxane (10 mL) was degassed and purged with N₂ three times. Then the mixture was stirred at 90° C. for 12 hrs under N₂ atmosphere. On completion, the mixture was concentrated to give a residue. The residue was purified by column chromatography (SiO₂, Petroleum ether/Ethyl acetate=10/1 to 5/1) to give the title compound (500 mg, 78% yield) as a yellow solid. LC-MS (ESI⁺) m/z 605.3 (M+H)⁺.

### Step 2—4-(4-((4-(4-(Tert-butoxycarbonyl)piperazin-1-yl)-4′-chloro-[1,1′-biphenyl]-2-yl)methyl)piperazin-1-yl)benzoic Acid

To a solution of tert-butyl 4-(4′-chloro-2-((4-(4-(methoxycarbonyl)phenyl)piperazin-1-yl)methyl)-[1,1′-biphenyl]-4-yl)piperazine-1-carboxylate (500 mg, 826 μmol) in THF (8 mL), MeOH (2 mL) and H₂O (2 mL) was added LiOH·H₂O (173 mg, 4.13 mmol). The mixture was then stirred at 50° C. for 12 hrs. On completion, the mixture was adjusted to pH=4 by adding 1M HCl solution. Then the mixture was extracted with DCM (30 mL×3), dried over anhydrous Na₂SO₄ and concentrated to give the title compound (330 mg) as a white solid. LC-MS (ESI⁺) m/z 591.0 (M+H)⁺.

### Synthesis of (R)-4-(4-((4′-chloro-4-(piperazin-1-yl)-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide (Intermediate LF)

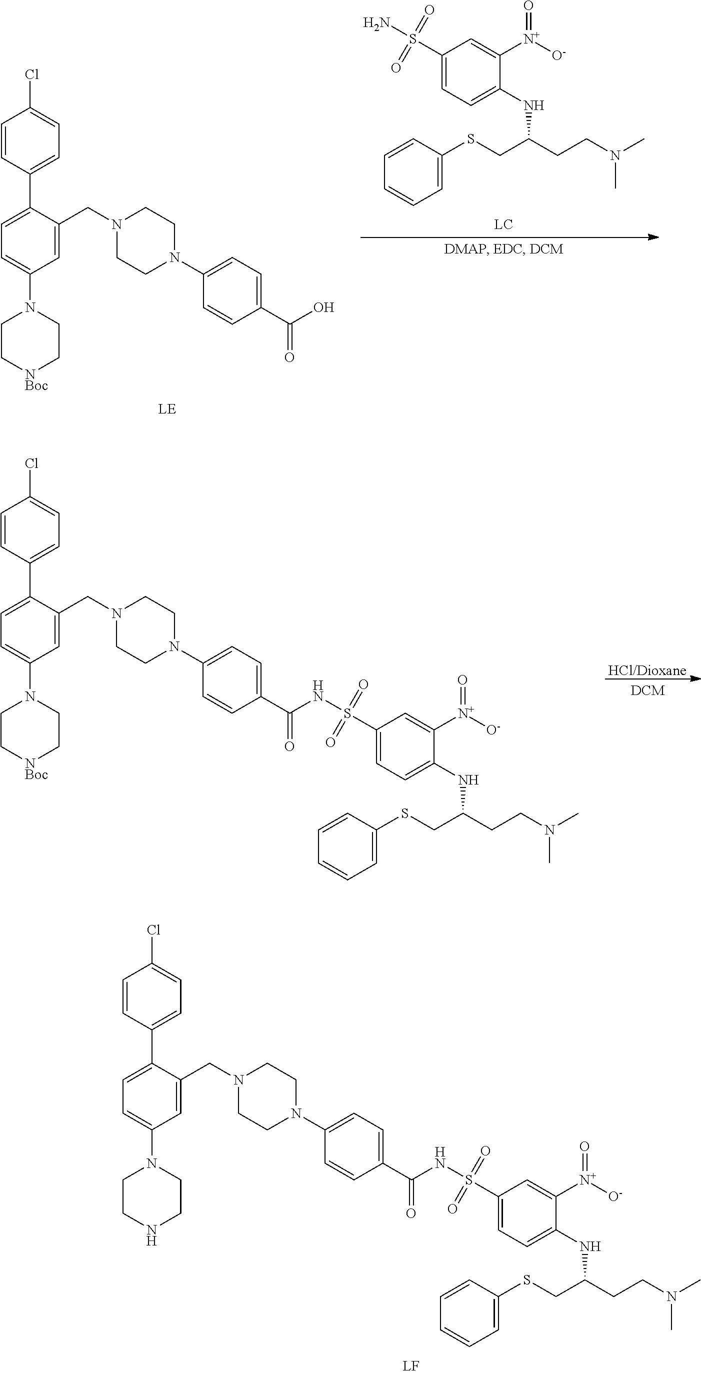 [SMILES: CC(C)(C)OC(=O)N1CCN(c2ccc(-c3ccc(Cl)cc3)c(CN3CCN(c4ccc(C(=O)O)cc4)CC3)c2)CC1 ; CN(C)CC[C@H](CSc1ccccc1)Nc1ccc(S(N)(=O)=O)cc1[N+](=O)[O-] ; CN(C)CC[C@H](CSc1ccccc1)Nc1ccc(S(=O)(=O)NC(=O)c2ccc(N3CCN(Cc4cc(N5CCN(C(=O)OC(C)(C)C)CC5)ccc4-c4ccc(Cl)cc4)CC3)cc2)cc1[N+](=O)[O-] ; "HCl/Dioxane" ; CN(C)CC[C@H](CSc1ccccc1)Nc1ccc(S(=O)(=O)NC(=O)c2ccc(N3CCN(Cc4cc(N5CCNCC5)ccc4-c4ccc(Cl)cc4)CC3)cc2)cc1[N+](=O)[O-]]

### Step 1—(R)-tert-butyl 4-(4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)piperazine-1-carboxylate

To a solution of 4-(4-((4-(4-(tert-butoxycarbonyl)piperazin-1-yl)-4′-chloro-[1,1′-biphenyl]-2-yl)methyl)piperazin-1-yl)benzoic acid (150 mg, 254 μmol, Intermediate LE) in DCM (4 mL) was added EDC (78.8 mg, 508 μmol, 89.8 uL) and DMAP (77.5 mg, 634 μmol). Then (R)-4 ((4-(dimethylamino)-1-(phenylthio)butan-2-yl)amino)-3-nitrobenzenesulfonamide (108 mg, 254 μmol, Intermediate LC) was added and the mixture was stirred at 30° C. for 6 hrs. On completion, the mixture was concentrated to give the crude product which was purified by reversed-phase HPLC (0.1% FA condition) to give the title compound (120 mg, 43% yield, FA) as a yellow solid. LC-MS (ESI⁺) m/z 499.2 (M/2+H)⁺.

### Step 2—(R)-4-(4-((4′-chloro-4-(piperazin-1-yl)-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide

To a solution of (R)-tert-butyl 4-(4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)piperazine-1-carboxylate (120 mg, 115 μmol, FA) in DCM (8 mL) was added HCl/EtOAc (4 M, 2 mL). The mixture was stirred at 20° C. for 2 hrs. On completion, the mixture was concentrated to give the title compound (150 mg, HCl) as a yellow solid. LC-MS (ESI⁺) m/z 897.3 (M+H)⁺.

### Synthesis of 8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoic Acid (Intermediate LG)

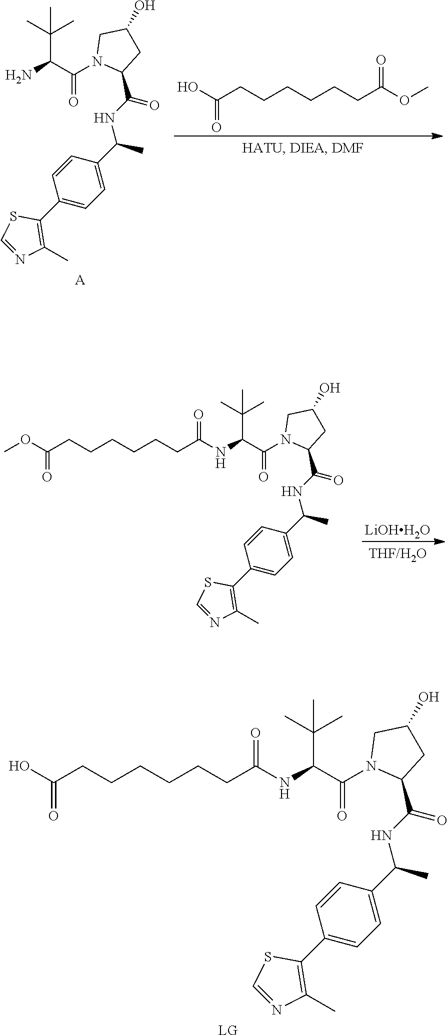

### Step 1—Methyl 8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoate

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (500 mg, 1.12 mmol, Intermediate A) in DMF (10 mL) was added DIEA (1.45 g, 11.25 mmol, 1.96 mL). Next, 8-methoxy-8-oxo-octanoic acid (211.68 mg, 1.12 mmol, 201.60 uL, CAS #3946-32-5) and HATU (470.38 mg, 1.24 mmol) was added at 0° C. and the mixture was stirred at 0° C. for 30 min. On completion, the reaction mixture was concentrated under reduced pressure to remove solvent. The crude product was purified by reversed-phase HPLC (0.1% FA condition) to give the title compound (550 mg, 80% yield) was obtained as a white solid. LC-MS (ESI⁺) m/z 615.3 (M+H)⁺.

### Step 2—8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoic Acid

To a solution of methyl 8-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-8-oxo-octanoate (500 mg, 813.28 μmol) in THF (5 mL) and H₂O (0.5 mL) was added LiOH H₂O (170.64 mg, 4.07 mmol). The mixture was stirred at 20° C. for 5 hr. On completion, the reaction mixture was concentrated under reduced pressure to remove the solvent. The crude product was purified by reversed-phase HPLC (0.1% FA condition) to give the title compound (480 mg, 98% yield) as a white solid. LC-MS (ESI⁺) m/z 601.6 (M+H)⁺.

### Synthesis of Tert-butyl 4-(4-bromo-3-formylbenzoyl)piperazine-1-carboxylate (Intermediate LH)

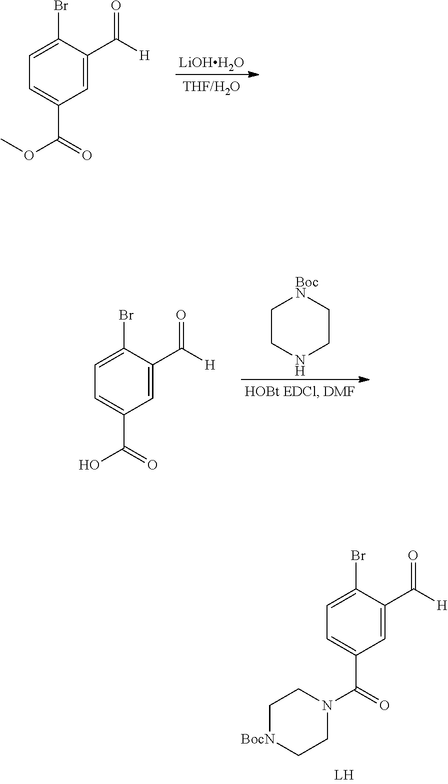

### Step 1—4-Bromo-3-formylbenzoic Acid

To a solution of methyl 4-bromo-3-formyl-benzoate (1 g, 4.11 mmol, CAS #858124-35-3) in THF (10 mL) and H₂O (1 mL) was added LiOH H₂O (492 mg, 20.5 mmol). The mixture was then stirred at 20° C. for 16 hrs. On completion, the reaction mixture was quenched with H₂O (10 mL). Then hydrochloric acid (8 mL, 1 mol/L) was added which precipitated a solid. The solid was filtered and dried under reduced pressure to give the give the title compound (800 mg) as white solid. LC-MS (ESI⁺) m/z 228 (M+H)⁺.

### Step 2—Tert-butyl 4-(4-bromo-3-formylbenzoyl)piperazine-1-carboxylate

To a solution of 4-bromo-3-formyl-benzoic acid (700 mg, 3.06 mmol) in DMF (12 mL) was added tert-butyl piperazine-1-carboxylate (378 mg, 1.70 mmol, HCl, CAS #143238-38-4). Then HOBt (917.76 mg, 6.79 mmol) and EDCI (1.63 g, 8.49 mmol) was added and the mixture was stirred at 20° C. for 5 hrs. On completion, the reaction mixture was concentrated under reduced pressure to remove solvent. The crude product was purified by reversed-phase HPLC (0.1% FA condition) give the title compound (340 mg, 50% yield) as a white solid. LC-MS (ESI⁺) m/z 296 (M-Boc)⁺.

### Synthesis of 4-(4-((4-(4-(Tert-butoxycarbonyl)piperazine-1-carbonyl)-4′-chloro-[1,1′-biphenyl]-2-yl)methyl)piperazin-1-yl)benzoic Acid (Intermediate LI)

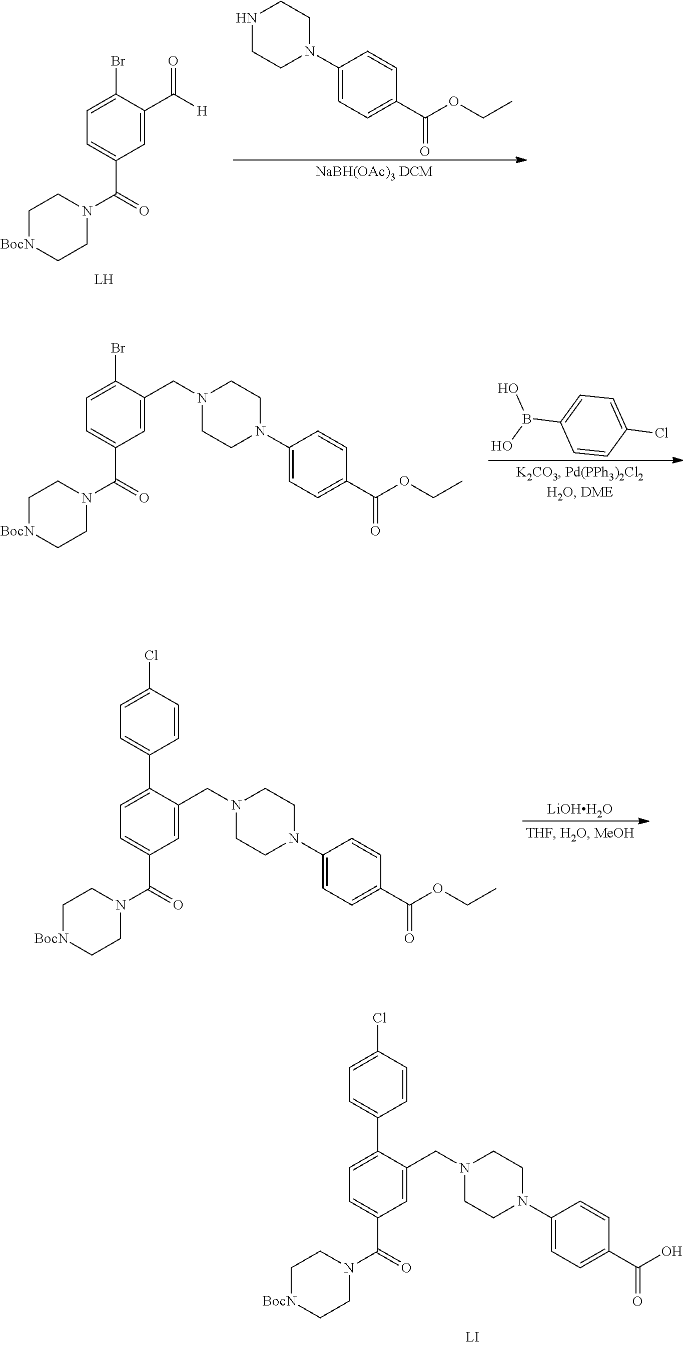

### Step 1—Tert-butyl 4-(4-bromo-3-((4-(4-(ethoxycarbonyl)phenyl)piperazin-1-yl)methyl)benzoyl)piperazine-1-carboxylate

To a solution of tert-butyl 4-(4-bromo-3-formyl-benzoyl)piperazine-1-carboxylate (290 mg, 730 μmol, Intermediate LH) in DCM (6 mL) was added ethyl 4-piperazin-1-ylbenzoate (188 mg, 802 μmol, CAS #80518-57-6) and NaBH(OAc) 3 (309 mg, 1.46 mmol). The mixture was stirred at 20° C. for 16 hr. On completion, the reaction mixture was quenched with H₂O (6 mL) at 20° C., and then extracted with DCM (10 mL×3). The combined organic layers were washed with H₂O (5 mL×3), dried over Na₂SO₄, filtered and concentrated under reduced pressure to give the crude product. The residue was purified by column chromatography (SiO₂, Petroleum ether/Ethyl acetate=3/1 to 1/1) to give the title compound (310 mg, 69% yield) as a white solid. LC-MS (ESI⁺) m/z 617.4 (M+H)⁺.

### Step 2—Tert-butyl 4-(4′-chloro-2-((4-(4-(ethoxycarbonyl)phenyl)piperazin-1-yl)methyl)-[1,1′-biphenyl]-4-carbonyl)piperazine-1-carboxylate

To a solution of tert-butyl 4-[4-bromo-3-[[4-(4-ethoxycarbonylphenyl)piperazin-1-yl]methyl]benzoyl]piperazine-1-carboxylate (250 mg, 406 μmol) in H₂O (1.5 mL) and DME (6 mL) was added (4-chlorophenyl)boronic acid (63.5 mg, 406 μmol, CAS #1678-18-1), Pd(PPh₃)₂Cl₂ (28.5 mg, 40.6 μmol) and K₂CO₃ (168 mg, 1.22 mmol). The mixture was degassed and purged with N₂ three times, and then the mixture was stirred at 80° C. for 16 hrs under N₂ atmosphere. On completion, the reaction mixture was partitioned between H₂O and DME. The organic phase was separated, washed with EA (10 mL×3), dried over Na₂SO₄, filtered and concentrated under reduced pressure to give a residue. The residue was purified by column chromatography (SiO₂, Petroleum ether/Ethyl acetate=10/1 to 1/1 R_{f}=0.2) to give the title compound (160 mg, 61% yield) as a white solid. LC-MS (ESI⁺) m/z 647.4 (M+H)⁺.

### Step 3—4-(4-((4-(4-(tert-butoxycarbonyl)piperazine-1-carbonyl)-4′-chloro-[1,1′-biphenyl]-2-yl)methyl)piperazin-1-yl)benzoic Acid

To a solution of tert-butyl 4-[[4-(4-chlorophenyl)-3-[4-(4-ethoxycarbonylphenyl)piperazin-1-yl]methyl]benzoyl]piperazine-1-carboxylate (150 mg, 231 μmol) in THF (2 mL) and H₂O (0.5 mL) and MeOH (0.5 mL) was added LiOH·H₂O (48.6 mg, 1.16 mmol). The mixture was stirred at 25° C. for 16 hrs. On completion, the reaction mixture was concentrated under reduced pressure to remove solvent. Then H₂O (10 mL) was added to the followed by hydrochloric acid (5 ml, 1 mol/L) where a solid precipitated. The reaction mixture was then partitioned between EA (30 mL) and H₂O (20 mL). The organic phase was separated, washed with H₂O (5 mL×3), dried over Na₂SO₄, filtered and concentrated under reduced pressure to give the crude product. The crude product was purified by reversed-phase HPLC (0.1% FA condition) to give the title compound (50 mg, 35% yield) as a white solid. LC-MS (ESI⁺) m/z 619.5 (M+H)⁺.

### Synthesis of (R)-4-(4-((4′-chloro-4-(piperazine-1-carbonyl)-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide (Intermediate LJ)

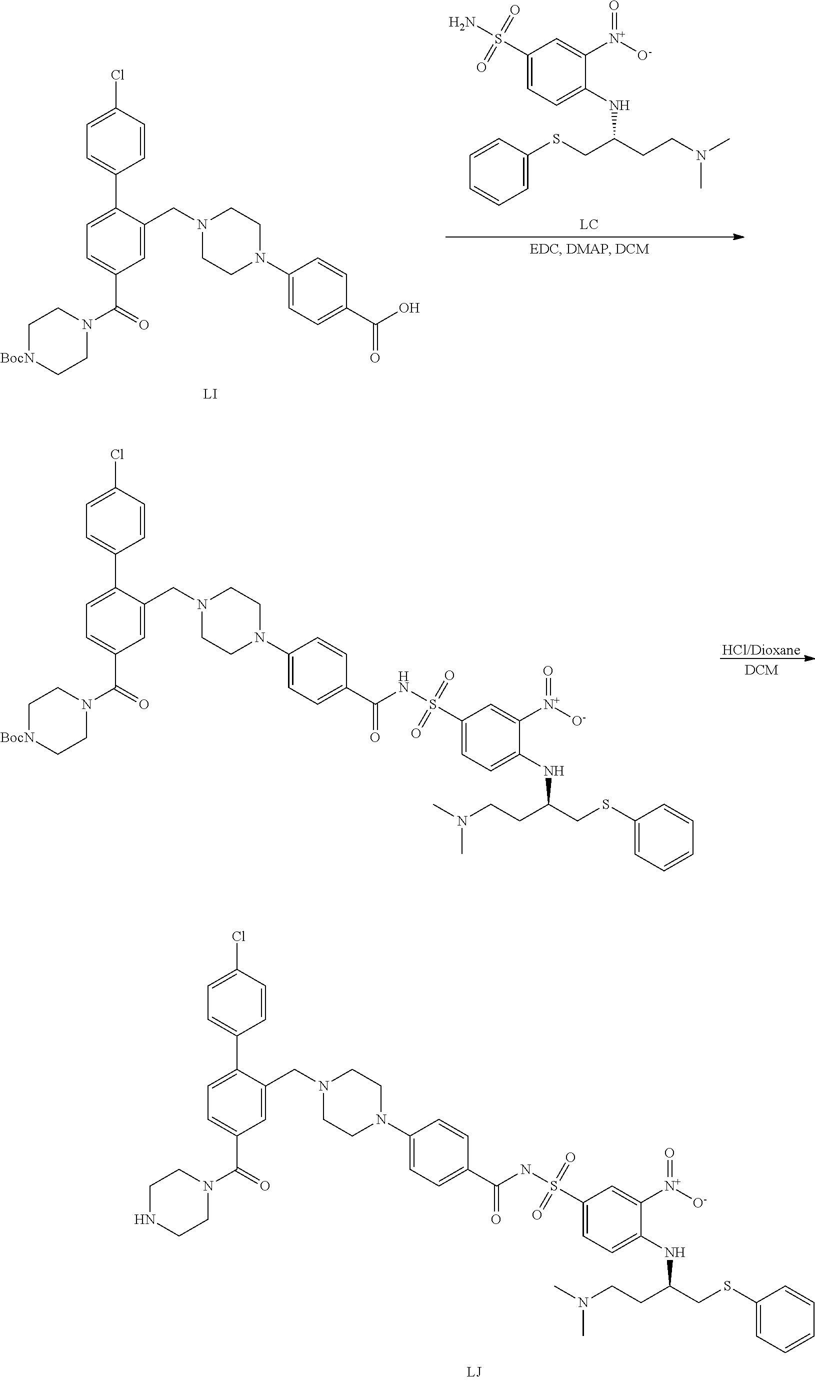

### Step 1—(R)-tert-butyl 4-(4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-carbonyl)piperazine-1-carboxylate

To a solution of 4-[4-[[5-(4-tert-butoxycarbonylpiperazine-1-carbonyl)-2-(4-chlorophenyl)phenyl]methyl]piperazin-1-yl]benzoic acid (50 mg, 80.7 μmol, Intermediate LI) in DCM (1 mL) was added EDC (25.0 mg, 161 μmol, 28.5 uL) and 4-[[(1R)-3-(dimethylamino)-1-(phenylsulfanylmethyl)propyl]amino]-3-nitro-benzenesulfonamide (37.7 mg, 88.8 μmol, Intermediate LC). Then DMAP (24.6 mg, 201 μmol) was added and the mixture was stirred at 25° C. for 16 hrs. On completion, the reaction mixture was quenched with H₂O (1 mL) at 20° C., and the reaction mixture was partitioned between H₂O and DCM. The organic phase was separated, washed with H₂O (2 mL×3), dried over Na₂SO₄, filtered and concentrated under reduced pressure to give the crude product. The crude product was purified by reversed-phase HPLC (0.1% FA condition) to give the title compound (26 mg, 31% yield) as a yellow solid. LC-MS (ESI⁺) m/z 1025.7 (M+H)⁺.

### Step 2—(R)-4-(4-((4′-chloro-4-(piperazine-1-carbonyl)-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide

To a solution of tert-butyl 4-[4-(4-chlorophenyl)-3-[[4-[4-[[4-[[(1R)-3-(dimethylamino)-1-(phenylsulfanylmethyl)propyl]amino]-3-nitro-phenyl]sulfonylcarbamoyl]phenyl]piperazin-1-yl]methyl]benzoyl]piperazine-1-carboxylate (26 mg, 25 μmol) in DCM (4 mL) was added HCl/dioxane (4 M, 6.34 uL). The mixture was stirred at 25° C. for 1 hr. On completion, the reaction mixture was concentrated under reduced pressure to remove solvent give the crude product. The crude product was purified by reversed-phase HPLC (0.1% HCl condition) to give the title compound (14 mg, 57% yield, HCl) as a yellow solid. LC-MS (ESI⁺) m/z 925.9 (M+H)⁺.

### Synthesis of 4-(4-((4-((4-(tert-butoxycarbonyl)piperazin-1-yl)methyl)-4′-chloro-[1,1′-biphenyl]-2-yl)methyl)piperazin-1-yl)benzoic Acid (Intermediate LK)

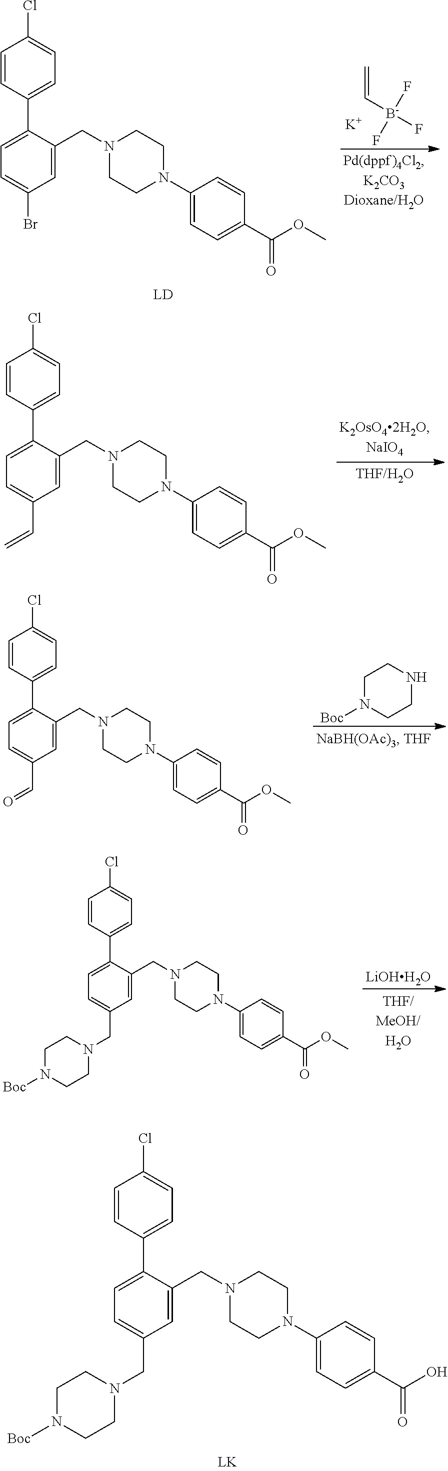

### Step 1—Methyl 4-(4-((4′-chloro-4-vinyl-[1,1′-biphenyl]-2-yl)methyl)piperazin-1-yl)benzoate

To a solution of methyl 4-(4-((4-bromo-4′-chloro-[1,1′-biphenyl]-2-yl)methyl)piperazin-1-yl)benzoate (860 mg, 1.72 mmol, Intermediate LD) and potassium trifluoro (vinyl) borate (345 mg, 2.58 mmol, CAS #13682-77-4) in dioxane (20 mL) and H₂O (5 mL) was added Pd(dppf)Cl₂ (125 mg, 172 μmol) and Cs₂CO₃ (1.68 g, 5.16 mmol). The mixture was then stirred at 80° C. for 5 hrs. On completion, the reaction mixture was quenched with water (50 mL) and extracted with ethyl acetate (2×25 mL). The combined organic layers were washed with brine (50 mL), dried over anhydrous sodium sulfate, filtered and the filtrate was concentrated in vacuo to give the crude residue. The residue was purified by column chromatography (SiO₂, Petroleum ether/Ethyl acetate=1/0 to 1/1) to give the title compound (681 mg, 89% yield,) as a white solid. LC-MS (ESI⁺) m/z 447.2 (M+H)⁺.

### Step 2—Methyl 4-(4-((4′-chloro-4-formyl-[1,1′-biphenyl]-2-yl)methyl)piperazin-1-yl)benzoate

To a solution of methyl 4-(4-((4′-chloro-4-vinyl-[1,1′-biphenyl]-2-yl)methyl)piperazin-1-yl)benzoate (680 mg, 1.52 mmol) in THF (20 mL) and H₂O (5 mL) was added Potassiumosmate(VI)dihydrate (39.2 mg, 106 μmol, CAS #10022-66-9) and sodium periodate (1.63 g, 7.61 mmol, CAS #7790-28-5). The mixture was then stirred at 25° C. for 2 hrs. On completion, the reaction mixture was quenched with aqueous Na₂SO₃ (20 mL) and then extracted with ethyl acetate (30 mL×3). The combined organic layers were washed by brine (60 mL) and dried over anhydrous sodium sulfate, filtered. The filtrate was concentrated in vacuo to give the title compound (800 mg) as a yellow solid. LC-MS (ESI⁺) m/z 449.3 (M+H)⁺.

### Step 3—Tert-butyl 4-((4′-chloro-2-((4-(4-(methoxycarbonyl)phenyl)piperazin-1-yl)methyl)-[1,1′-biphenyl]-4-yl)methyl)piperazine-1-carboxylate

To a solution of methyl 4-(4-((4′-chloro-4-formyl-[1,1′-biphenyl]-2-yl)methyl)piperazin-1-yl)benzoate (780 mg, 1.74 mmol) and tert-butyl piperazine-1-carboxylate (485 mg, 2.61 mmol) in DCM (20 mL) was added NaBH(OAc)₃ (368 mg, 1.74 mmol). The mixture was stirred at 25° C. for 12 hrs. On completion, the reaction mixture was quenched with water (2 mL) and extracted by dichloromethane (5 mL). The combined organic layers were washed by brine (10 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 1/1).to give the title compound (600 mg, 55% yield) as a white solid. LC-MS (ESI⁺) m/z 447.2 (M+H)⁺. LC-MS (ESI⁺) m/z 619.7 (M+H)⁺.

### Step 4—4-(4-((4-((4-(Tert-butoxycarbonyl)piperazin-1-yl)methyl)-4′-chloro-[1,1′-biphenyl]-2-yl)methyl)piperazin-1-yl)benzoic Acid

To a solution of tert-butyl 4-((4′-chloro-2-((4-(4-(methoxycarbonyl)phenyl)piperazin-1-yl)methyl)-[1,1′-biphenyl]-4-yl)methyl)piperazine-1-carboxylate (600 mg, 969 μmol) in THF (12 mL), MeOH (3 mL) and H₂O (3 mL) was added LiOH·H₂O (203 mg, 4.85 mmol). The mixture was stirred at 50° C. for 24 hrs. On completion, the reaction mixture was added HCl (1N) until the pH=6.0, then diluted by water (5 mL) and extracted by dichloromethane (2×20 mL). The combined organic layers were dried over anhydrous sodium sulfate, filtered and the filtrate was concentrated in vacuo to give the title compound (560 mg) as a pink solid. LC-MS (ESI⁺) m/z 605.4 (M+H)⁺.

### Synthesis of (R)-4-(4-((4′-chloro-4-(piperazin-1-ylmethyl)-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide (Intermediate LL)

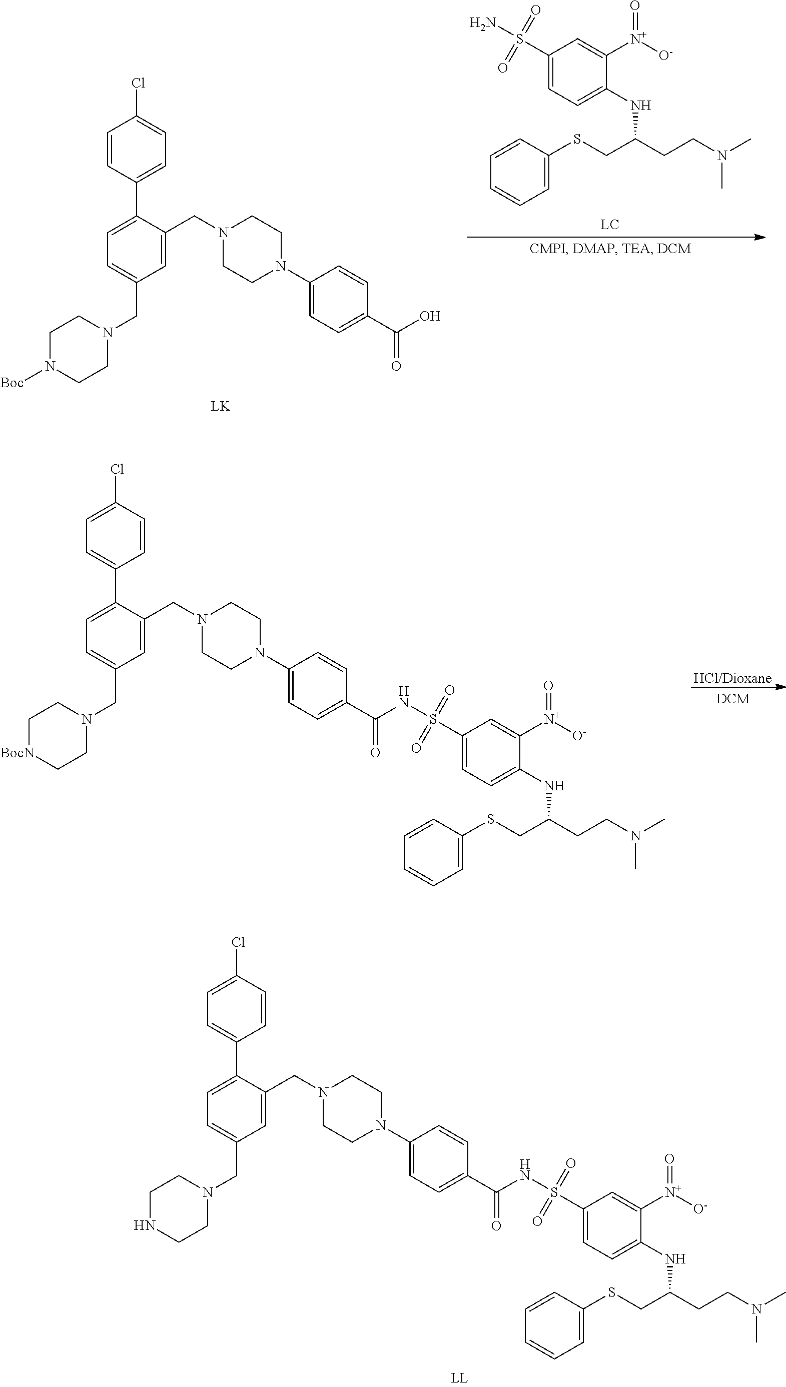 [SMILES: CC(C)(C)OC(=O)N1CCN(Cc2ccc(-c3ccc(Cl)cc3)c(CN3CCN(c4ccc(C(=O)O)cc4)CC3)c2)CC1 ; CN(C)CC[C@H](CSc1ccccc1)Nc1ccc(S(N)(=O)=O)cc1[N+](=O)[O-] ; CN(C)CC[C@H](CSc1ccccc1)Nc1ccc(S(=O)(=O)NC(=O)c2ccc(N3CCN(Cc4cc(CN5CCN(C(=O)OC(C)(C)C)CC5)ccc4-c4ccc(Cl)cc4)CC3)cc2)cc1[N+](=O)[O-] ; "HCl/Dioxane" ; CN(C)CC[C@H](CSc1ccccc1)Nc1ccc(S(=O)(=O)NC(=O)c2ccc(N3CCN(Cc4cc(CN5CCNCC5)ccc4-c4ccc(Cl)cc4)CC3)cc2)cc1[N+](=O)[O-]]

### Step 1—(R)-tert-butyl 4-((4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)methyl)piperazine-1-carboxylate

To a solution of 4-(4-((4-((4-(tert-butoxycarbonyl)piperazin-1-yl)methyl)-4′-chloro-[1,1′-biphenyl]-2-yl)methyl)piperazin-1-yl)benzoic acid (160 mg, 264 μmol, Intermediate LK) in DCM (2 mL) was added DMAP (32.3 mg, 264 μmol) 2-chloro-1-methyl-pyridin-1-ium iodide (101 mg, 396 μmol) TEA (80.2 mg, 793 μmol, 110 uL). Then the mixture was stirred for 5 min, and (R)-4-((4-(dimethylamino)-1-(phenylthio)butan-2-yl)amino)-3-nitrobenzenesulfonamide (168 mg, 396 μmol, Intermediate LC) was added. The mixture was stirred at 25° C. for 12 hrs. On completion, the reaction mixture was concentrated under reduced pressure to remove the solvent. The crude was then purified by reversed-phase HPLC (0.1% FA condition) to give the title compound (200 mg, 72% yield, FA) as a yellow solid LC-MS (ESI⁺) m/z 1011.9 (M+H)⁺.

### Step 2—(R)-4-(4-((4′-chloro-4-(piperazin-1-ylmethyl)-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide

To a solution of (R)-tert-butyl 4-((4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)methyl)piperazine-1-carboxylate (200 mg, 189 μmol, FA) in DCM (10 mL) was added HCl/dioxane (4 M, 1 mL). The mixture was then stirred at 25° C. for 0.2 hr. On completion, the reaction mixture was concentrated under reduced pressure to remove solvent and give the title compound (200 mg, HCl) as a yellow solid. LC-MS (ESI⁺) m/z 911.5 (M+H)⁺.

### Synthesis of (2S,4R)-1-((S)-2-(1-fluorocyclopropanecarboxamido)-3,3-dimethylbutanoyl)-4-hydroxy-N-(4-(4-methylthiazol-5-yl)-2-(2-oxoethoxy)benzyl)pyrrolidine-2-carboxamide (Intermediate LM)

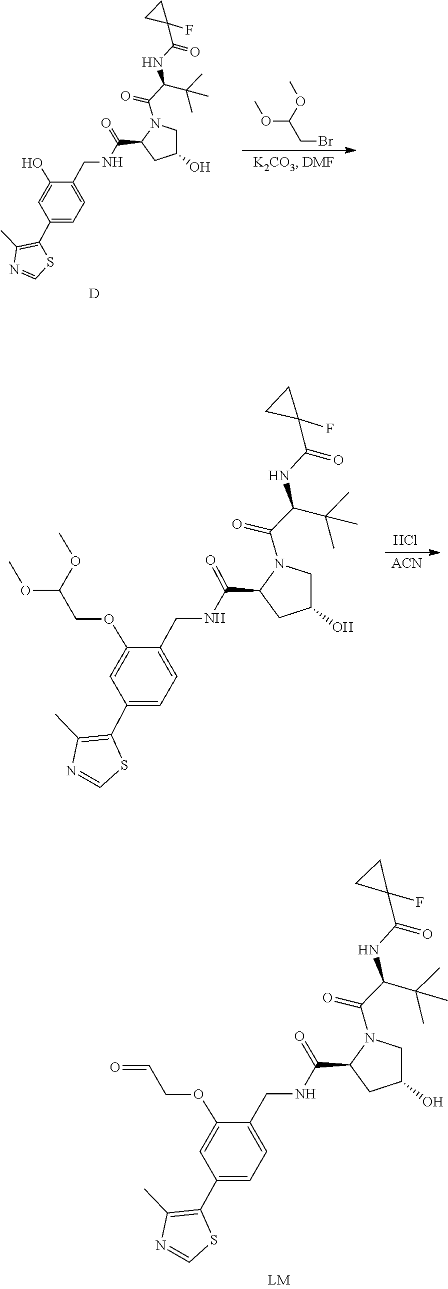

### Step 1—(2S,4R)—N-(2-(2,2-dimethoxyethoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide

To a solution of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (150 mg, 281 μmol, Intermediate D) and 2-bromo-1,1-dimethoxyethane (143 mg, 845 μmol, 99.2 uL) in DMF (1.5 mL) was added K₂CO₃ (117 mg, 845 μmol). The mixture was then stirred at 70° C. for 12 h. On completion, The reaction mixture was poured into ice water (5 mL) and extracted with EtOAc (5 mL×3). The combined organic layers were washed with brine (5 mL×3), dried over sodium sulfate, filtered and concentrated under reduced pressure to give a residue. The residue was purified by column chromatography (SiO₂, Petroleum ether/Ethyl acetate=1/0 to 0/1) to give the title compound (130 mg, 74% yield) as a white solid. ¹H NMR (400 MHz, DMSO-d₆) δ 8.98 (s, 1H), 8.48 (br t, J=5.8 Hz, 1H), 7.41 (d, J=7.8 Hz, 1H), 7.29 (dd, J=2.4, 8.8 Hz, 1H), 7.06 (d, J=1.12 Hz, 1H), 7.00-6.96 (m, 1H), 5.17 (d, J=3.6 Hz, 1H), 4.76-4.69 (m, 1H), 4.60 (d, J=9.2 Hz, 1H), 4.51 (t, J=8.4 Hz, 1H), 4.38-4.27 (m, 2H), 4.24-4.16 (m, 1H), 4.07 (d, J=5.2 Hz, 2H), 3.68-3.57 (m, 2H), 3.39-3.36 (m, 6H), 2.46 (s, 3H), 2.14-2.05 (m, 1H), 1.96-1.87 (m, 1H), 1.42-1.31 (m, 2H), 1.24-1.16 (m, 2H), 0.96 (s, 9H).

### Step 2—(2S,4R)-1-((S)-2-(1-fluorocyclopropanecarboxamido)-3,3-dimethylbutanoyl)-4-hydroxy-N-(4-(4-methylthiazol-5-yl)-2-(2-oxoethoxy)benzyl)pyrrolidine-2-carboxamide

To a solution of (2S,4R)—N-(2-(2,2-dimethoxyethoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (120 mg, 193 μmol) in ACN (1 mL) was added HCl (2 M, 1.56 mL). The mixture was then stirred at 50° C. for 12 h. On completion, the reaction mixture was poured into NaHCO₃ (sat, aq, 5 mL) and extracted with EtOAc (5 mL×3). The combined organic layers were washed with brine (5 mL×3), dried over sodium sulfate, filtered and concentrated under reduced pressure to give the title compound (90 mg) as a white solid. LC-MS (ESI⁺) m/z 575.23 (M+H)⁺.

### Synthesis of (S)-4-(2-(2-(2-cyclopropylphenyl)pyrrolidin-1-yl)-7-azaspiro[3.5]nonan-7-yl)benzoic Acid (Intermediate LN)

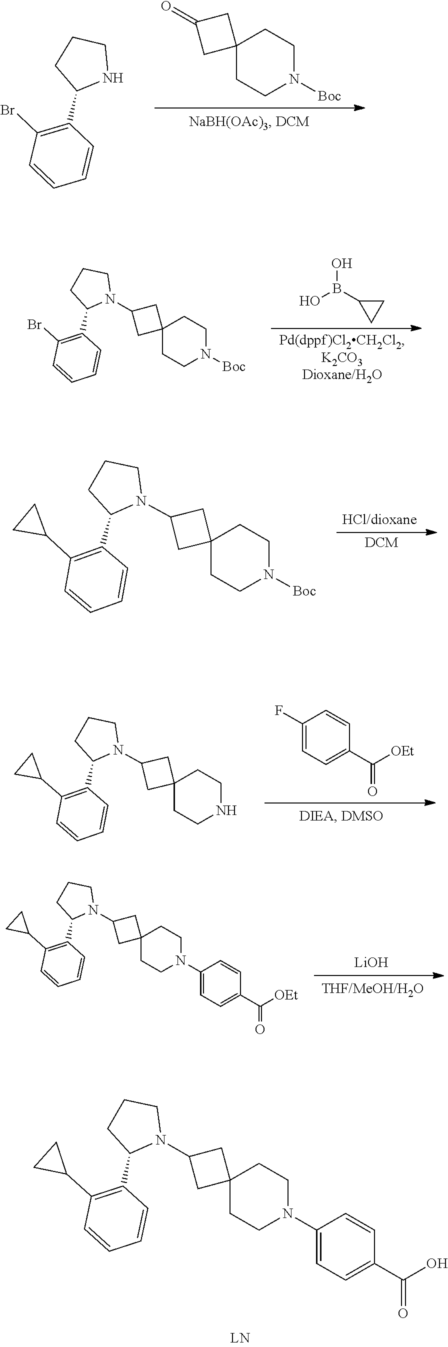

### Step 1—(S)-tert-butyl 2-(2-(2-bromophenyl)pyrrolidin-1-yl)-7-azaspiro[3.5]nonane-7-carboxylate

To a solution of (S)-2-(2-bromophenyl)pyrrolidine (1.7 g, 7.52 mmol, CAS #1217680-26-6), tert-butyl 2-oxo-7-azaspiro[3.5]nonane-7-carboxylate (1.80 g, 7.52 mmol, CAS #203661-69-2) in DCM (17 mL) was added NaBH(OAc)₃ (3.20 g, 15.1 mmol), then the mixture was stirred at 25° C. for 2 hrs. On completion, the mixture was concentrated under reduced pressure and purified by column chromatography (SiO₂, Petroleum ether/Ethylacetate=0/1 to Dichloromethane/Methanol=20/1) to give the compound (3.2 g, 95% yield) as a brown oil. LC-MS (ESI⁺) m/z 449.3 (M+H)⁺.

### Step 2—(S)-tert-butyl 2-(2-(2-cyclopropylphenyl)pyrrolidin-1-yl)-7-azaspiro[3.5]nonane-7-carboxylate

To a solution of tert-butyl (S)-2-(2-(2-bromophenyl)pyrrolidin-1-yl)-7-azaspiro[3.5]nonane-7-carboxylate (3 g, 6.68 mmol) and cyclopropylboronic acid (2.87 g, 33.3 mmol, CAS #411235-57-9) in H₂O (3 mL) and dioxane (27 mL) was added K₂CO₃ (5.54 g, 40.0 mmol) and Pd(dppf)Cl₂·CH₂Cl₂ (545 mg, 667 μmol) under N₂, Then the mixture was stirred at 100° C. for 4 hrs. On completion, the reaction was poured into water (30 mL) and extracted with EtOAc (50 mL×3). The combined organic phase was washed with brine (50 mL×2), dried over Na₂SO₄, filtered and the filtrate was concentrated to give a residue. The residue was purified by column chromatography (SiO₂, Petroleum ether:Ethyl Acetate=5:1) to give the title compound (2.1 g, 77% yield) as a white solid. LC-MS (ESI⁺) m/z 411.9 (M+H)⁺.

### Step 3—(S)-2-(2-(2-cyclopropylphenyl)pyrrolidin-1-yl)-7-azaspiro[3.5]nonane

To tert-butyl (S)-2-(2-(2-cyclopropylphenyl)pyrrolidin-1-yl)-7-a solution of azaspiro[3.5]nonane-7-carboxylate (2 g, 4.87 mmol) in DCM (20 mL) was added HCl/dioxane (4 M, 20 mL). The mixture was then stirred at 25° C. for 2 hrs. On completion, the reaction mixture was concentrated under reduced pressure to give the title compound (2.2 g) as a brown oil. LC-MS (ESI⁺) m/z 311.3 (M+H)⁺.

### Step 4—(S)-ethyl 4-(2-(2-(2-cyclopropylphenyl)pyrrolidin-1-yl)-7-azaspiro[3.5]nonan-7-yl)benzoate

A mixture of (S)-2-(2-(2-cyclopropylphenyl)pyrrolidin-1-yl)-7-azaspiro[3.5]nonane (1.5 g, 4.83 mmol), ethyl 4-fluorobenzoate (3.25 g, 19.3 mmol, CAS #451-46-7), and DIEA (9.37 g, 72.4 mmol, 12.6 mL) in DMSO (40 mL) was degassed and purged with N₂ three times. Then the mixture was stirred at 120° C. for 12 hrs under N₂ atmosphere. On completion, the reaction mixture was diluted with H₂O (40 mL) and extracted with EA (40 mL×3). The combined organic layers were washed with brine (40 mL×3), dried over Na₂SO₄, filtered and the filtrate was concentrated under reduced pressure to give a residue. The residue was purified by column chromatography (SiO₂, Petroleum ether/Ethyl acetate=1/0 to 1/1) to give the title compound (2.2 g) as a black solid. LC-MS (ESI⁺) m/z 459.9 (M+H)⁺.

### Step 5—(S)-4-(2-(2-(2-cyclopropylphenyl)pyrrolidin-1-yl)-7-azaspiro[3.5]nonan-7-yl)benzoic Acid

To a solution of ethyl(S)-4-(2-(2-(2-cyclopropylphenyl)pyrrolidin-1-yl)-7-azaspiro[3.5]nonan-7-yl)benzoate (1 g, 2.18 mmol) in H₂O (3 mL), THF (12 mL) and MeOH (3 mL) was added LiOH·H₂O (274 mg, 6.54 mmol). The mixture was then stirred at 25° C. for 12 hrs. On completion, the reaction mixture was concentrated under reduced pressure to give a residue. The residue was purified by prep-HPLC (FA condition) to give the title compound (200 mg, 18% yield) as a white solid. LC-MS (ESI⁺) m/z 431.2 (M+H)⁺.

### Synthesis of 4-(2-((S)-2-(2-cyclopropylphenyl)pyrrolidin-1-yl)-7-azaspiro[3.5]nonan-7-yl)-N-((4-((R)-1-(phenylthio)-4-(piperazin-1-yl)butan-2-yl)amino)-3-((trifluoromethyl)sulfonyl)phenyl)sulfonyl)benzamide (Intermediate LO)

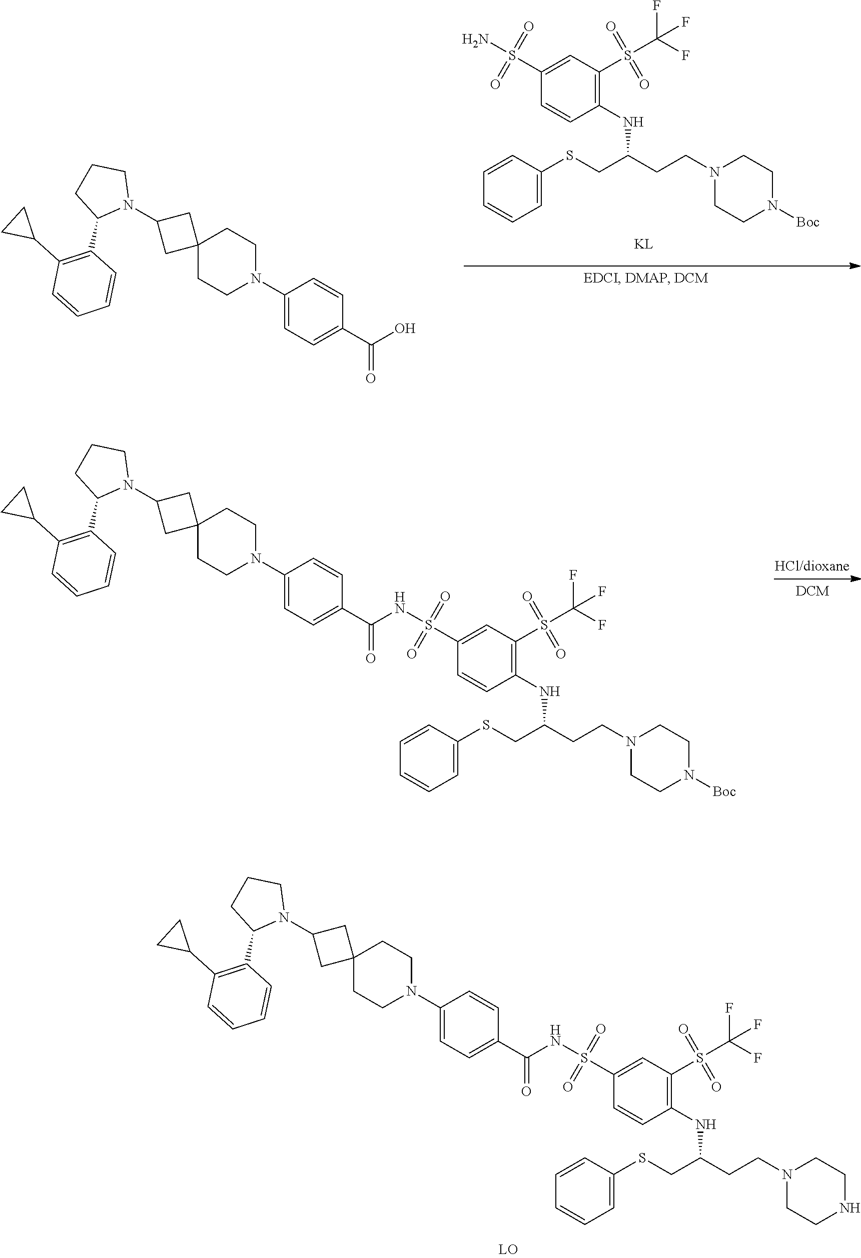 [SMILES: O=C(O)c1ccc(N2CCC3(CC2)CC(N2CCC[C@H]2c2ccccc2C2CC2)C3)cc1 ; CC(C)(C)OC(=O)N1CCN(CC[C@H](CSc2ccccc2)Nc2ccc(S(N)(=O)=O)cc2S(=O)(=O)C(F)(F)F)CC1 ; CC(C)(C)OC(=O)N1CCN(CC[C@H](CSc2ccccc2)Nc2ccc(S(=O)(=O)NC(=O)c3ccc(N4CCC5(CC4)CC(N4CCC[C@H]4c4ccccc4C4CC4)C5)cc3)cc2S(=O)(=O)C(F)(F)F)CC1 ; O=C(NS(=O)(=O)c1ccc(N[C@H](CCN2CCNCC2)CSc2ccccc2)c(S(=O)(=O)C(F)(F)F)c1)c1ccc(N2CCC3(CC2)CC(N2CCC[C@H]2c2ccccc2C2CC2)C3)cc1]

### Step 1—Tert-butyl 4-((R)-3-((4-(N-(4-(2-((S)-2-(2-cyclopropylphenyl)pyrrolidin-1-yl)-7-azaspiro[3.5]nonan-7-yl)benzoyl)sulfamoyl)-2-((trifluoromethyl)sulfonyl)phenyl)amino)-4-(phenylthio)butyl)piperazine-1-carboxylate

To a solution of (S)-4-(2-(2-(2-cyclopropylphenyl)pyrrolidin-1-yl)-7-azaspiro[3.5]nonan-7-yl)benzoic acid (50 mg, 116 μmol, Intermediate LN) in DCM (1 mL) was added EDCI (29.6 mg, 154 μmol), DMAP (23.6 mg, 193 μmol) and tert-butyl (R)-4-(4-(phenylthio)-3-((4-sulfamoyl-2-((trifluoromethyl)sulfonyl)phenyl)amino)butyl)piperazine-1-carboxylate (50.5 mg, 77.4 μmol, Intermediate KL). The mixture was then stirred at 20° C. for 2 hrs. On completion, the reaction mixture was concentrated under reduced pressure to give a residue. The residue was purified by prep-HPLC (FA condition) to give the title compound (60 mg, 33% yield) as a white solid. LC-MS (ESI+) m/z 1065.8 (M+H)⁺.

### Step 2—4-(2-((S)-2-(2-cyclopropylphenyl)pyrrolidin-1-yl)-7-azaspiro[3.5]nonan-7-yl)-N-((4-(((R)-1-(phenylthio)-4-(piperazin-1-yl)butan-2-yl)amino)-3-((trifluoromethyl)sulfonyl)phenyl)sulfonyl)benzamide

To a solution of tert-butyl 4-((R)-3-((4-(N-(4-(2-((S)-2-(2-cyclopropylphenyl)pyrrolidin-1-yl)-7-azaspiro[3.5]nonan-7-yl)benzoyl)sulfamoyl)-2-((trifluoromethyl)sulfonyl)phenyl)amino)-4-(phenylthio)butyl)piperazine-1-carboxylate (60 mg, 56.3 μmol) in DCM (1 mL) was added HCl/Dioxane (4 M, 14.0 uL). The mixture was then stirred at 25° C. for 2 hrs. On completion, the reaction mixture was concentrated under reduced pressure to give the title compound (40 mg, HCl) as a yellow solid. LC-MS (ESI+) m/z 965.4 (M+H)⁺.

### Synthesis of 9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoic Acid (Intermediate LP)

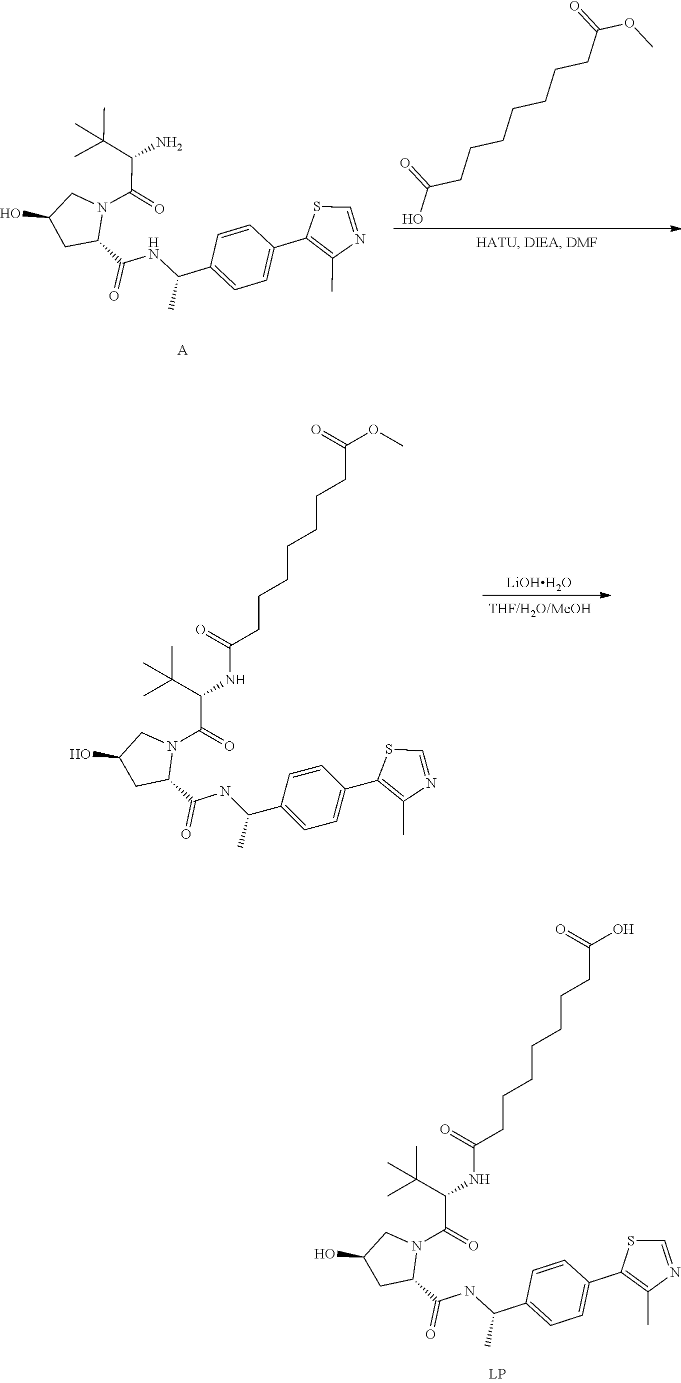

### Step 1—Methyl 9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoate

To a solution of 9-methoxy-9-oxononanoic acid (273 mg, 1.35 mmol, CAS #2104-19-0) in DMF (10 mL) was added (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (500 mg, 1.12 mmol, Intermediate A), HATU (470 mg, 1.24 mmol) and DIEA (1.45 g, 11.3 mmol, 1.96 mL). The mixture was then stirred at 25° C. for 2 hrs. On completion, the reaction mixture was partitioned between H₂O (40 mL) and DMF (10 mL), then extracted by ethyl acetate (150 mL). The organic layer was then dried over anhydrous sodium sulfate, filtered and the filtrate was concentrated under reduced pressure to give a residue. The residue was purified by column chromatography (SiO₂, Petroleum ether/Ethyl acetate=2/1 to 0/1, DCM:EA=1:1) to give the title compound (540 mg, 76% yield) as a white solid. LC-MS (ESI⁺) m/z 629.6 (M+H)⁺.

### Step 2—9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoic Acid

To a solution of methyl 9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoate (500 mg, 795 μmol) in THF (8 mL), MeOH (2 mL), and H₂O (2 mL) was added LiOH·H₂O (167 mg, 3.98 mmol). The mixture was then stirred at 25° C. for 12 hrs. On completion, the reaction mixture was quenched with 1M HCl at 25° C. until the pH=5, and then diluted with water (5 mL) and extracted with dichloromethane (25 mL). The organic layer was dried over anhydrous sodium sulfate, filtered and the filtrate was concentrated in vacuo to give the title compound (430 mg) as a white solid. LC-MS (ESI⁺) m/z 615.3 (M+H)⁺.

### Synthesis of 4-(4-((4-((Tert-butoxycarbonyl)amino)-4′-chloro-[1,1′-biphenyl]-2-yl)methyl)piperazin-1-yl)benzoic Acid (Intermediate LO)

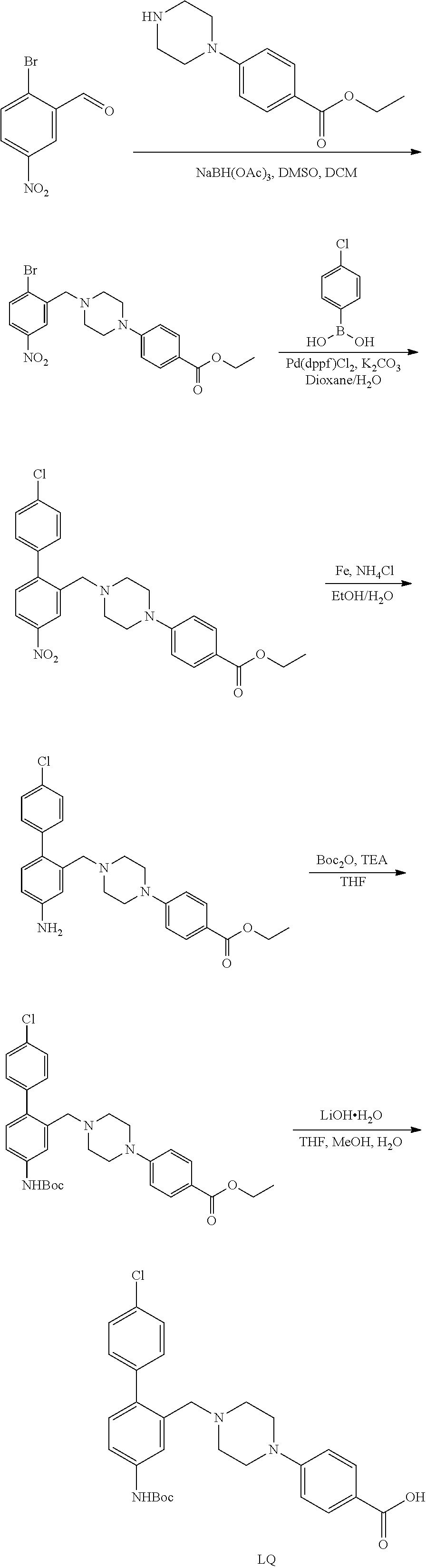

### Step 1—Ethyl 4-(4-(2-bromo-5-nitrobenzyl)piperazin-1-yl)benzoate

To a solution of 2-bromo-5-nitrobenzaldehyde (3 g, 13.0 mmol, CAS #84459-32-5) and ethyl 4-(piperazin-1-yl)benzoate (3.67 g, 15.7 mmol, CAS #80518-57-6) in DCM (80 mL) was added NaBH(OAc) 3 (6.91 g, 32.6 mmol). The mixture was then stirred at 25° C. for 12 hrs. On completion, the reaction mixture was quenched with water (100 mL) at 25° C., and then filtered to give a filter cake. The filter cake was triturated with H₂O at 25° C. for 15 min then filtered and dried to give the tittle compound (4 g) as a yellow solid. LC-MS (ESI⁺) m/z 450.2 (M+H)⁺.

### Step 2—Ethyl 4-(4-((4′-chloro-4-nitro-[1,1′-biphenyl]-2-yl)methyl)piperazin-1-yl)benzoate

A mixture of ethyl 4-(4-(2-bromo-5-nitrobenzyl)piperazin-1-yl)benzoate (4 g, 8.92 mmol). (4-chlorophenyl)boronic acid (1.53 g, 9.81 mmol, CAS #1679-18-1), Pd(dppf)Cl₂ (653 mg, 892 μmol), and K₂CO₃ (3.70 g, 26.8 mmol) in H₂O (40 mL) and dioxane (10 mL) was degassed and purged with N₂ three times. Then the mixture was stirred at 60° C. for 2 hrs under N atmosphere. On completion, the reaction mixture was concentrated under reduced pressure to give the crude product. The crude product was purified by column chromatography (SiO₂, Petroleum ether/Ethyl acetate=10/1 to 4/1) to give the tittle compound (3 g, 64% yield) as a brown solid. LC-MS (ESI⁺) m/z 480.3 (M+H)⁺.

### Step 3—Ethyl 4-(4-((4-amino-4′-chloro-[1,1′-biphenyl]-2-yl)methyl)piperazin-1-yl)benzoate

To a solution of ethyl 4-(4-((4′-chloro-4-nitro-[1,1′-biphenyl]-2-yl)methyl)piperazin-1-yl)benzoate (3 g, 6.25 mmol) in EtOH (40 mL) and H₂O (15 mL) was added NH₂Cl (1.34 g, 25.0 mmol) and Fc (1.40 g, 25.0 mmol). The mixture was then stirred at 50° C. for 12 hrs. On completion, the reaction mixture was filtered and the filtrate was concentrated under reduced pressure to give a crude product. The crude product was purified by column chromatography (SiO₂, Petroleum ether/Ethyl acetate=5/1 to 3/1. DCM:MeOH=20:1) to give the title compound (2 g, 61% yield) as a brown solid. LC-MS (ESI⁺) m/z 450.1 (M+H)⁺.

### Step 4—Ethyl 4-(4-((4-((tert-butoxycarbonyl)amino)-4′-chloro-[1,1′-biphenyl]-2-yl)methyl)piperazin-1-yl)benzoate

A mixture of ethyl 4-(4-((4-amino-4′-chloro-[1,1′-biphenyl]-2-yl)methyl)piperazin-1-yl)benzoate (600 mg, 1.33 mmol). TEA (405 mg, 4.00 mmol, 557 uL), and Boc₂O (873 mg, 4.00 mmol, 920 uL) in THF (15 mL) was degassed and purged with N₂ three times. Then the mixture was stirred at 0° C. for 12 hrs under N₂ atmosphere. On completion, the reaction mixture was concentrated under reduced pressure to give a crude product. The crude product was purified by column chromatography (SiO₂, Petroleum ether/Ethyl acetate=10/1 to 5/1) to give the title compound (550 mg, 73% yield) as a gray solid. LC-MS (ESI⁺) m/z 550.4 (M+H)⁺.

### Step 5—4-(4-((4-((tert-butoxycarbonyl)amino)-4′-chloro-[1,1′-biphenyl]-2-yl)methyl)piperazin-1-yl)benzoic Acid

To a solution of ethyl 4-(4-((4-((tert-butoxycarbonyl)amino)-4′-chloro-[1,1′-biphenyl]-2-yl)methyl)piperazin-1-yl)benzoate (300 mg, 545 μmol) in THF (3 mL) was added LiOH·H₂O (114 mg. 2.73 mmol). MeOH (3 mL) and H₂O (2 mL). The mixture was stirred at 50° C. for 12 hrs. On completion, the reaction mixture was quenched with 1M HCl at 25° C. until the pH=5, and then diluted with water (20 mL) and extracted with dichloromethane (60 mL). The organic layer was then concentrated under reduced pressure to give the title compound (240 mg) as a gray solid. LC-MS (ESI⁺) m/z 522.5 (M+H)⁺.

### Synthesis of (R)-4-(4-((4-amino-4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide (Intermediate LR)

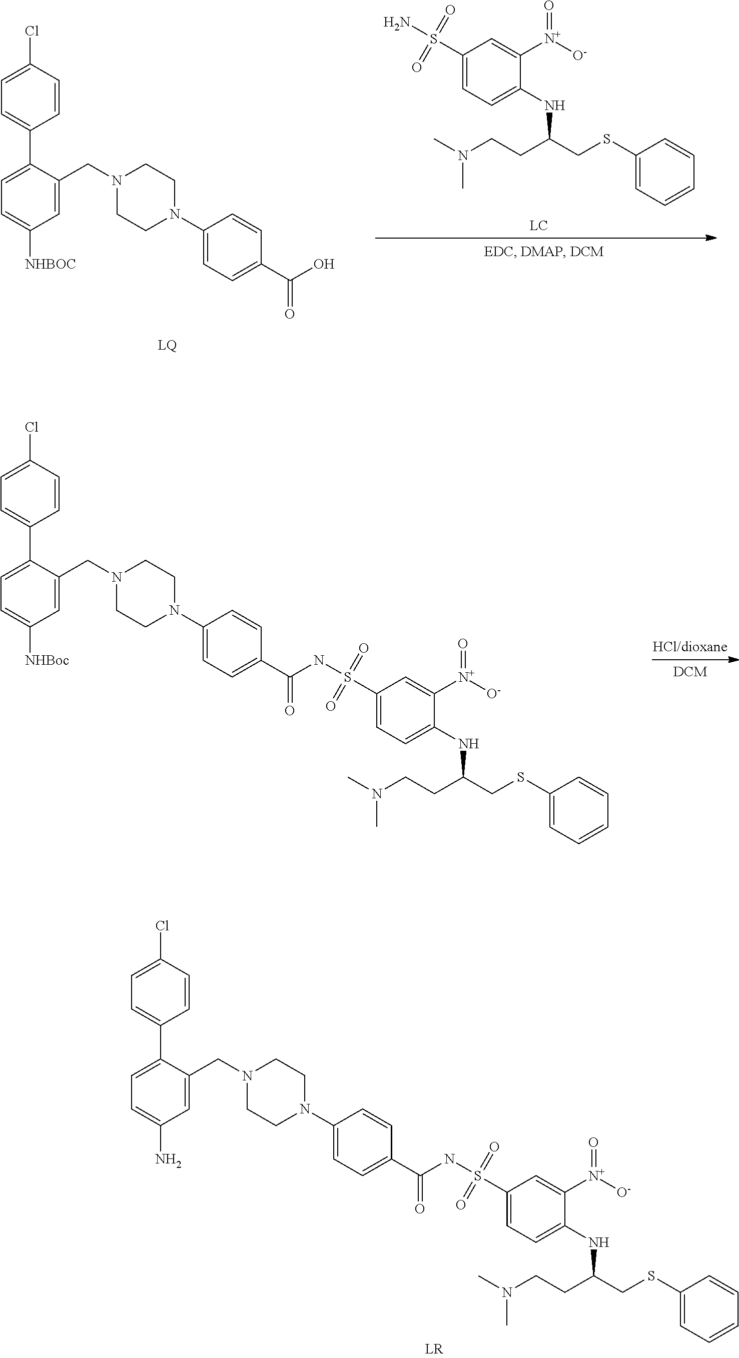

### Step 1—(R)-tert-butyl (4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)carbamate

To a solution of 4-(4-((4-((tert-butoxycarbonyl)amino)-4′-chloro-[1,1′-biphenyl]-2-yl)methyl)piperazin-1-yl)benzoic acid (240 mg, 460 μmol, Intermediate LQ) in DCM (6 mL) was added (R)-4-((4-(dimethylamino)-1-(phenylthio)butan-2-yl)amino)-3-nitrobenzenesulfonamide (180 mg, 424 μmol, Intermediate LC), DMAP (130 mg, 1.06 mmol) and EDC (165 mg, 1.06 mmol, 187 uL). The mixture was then stirred at 25° C. for 12 hrs. On completion, the reaction mixture was concentrated under reduced pressure to give a residue, the residue was purified by reversed-phase HPLC (0.1% FA condition) to give the title compound (100 mg, 24% yield, FA) as a yellow solid. LC-MS (ESI⁺) m/z 928.7 (M+H)⁺.

### Step 2—(R)-4-(4-((4-amino-4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide

To a solution of (R)-tert-butyl (4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)carbamate (95 mg, 102 μmol) in DCM (1 mL) was added HCl/dioxane (4 M, 3.80 mL). The mixture was stirred at 25° C. for 1 hr. On completion, the reaction mixture was concentrated under reduced pressure to give the title compound (100 mg) as a yellow solid. LC-MS (ESI⁺) m/z 828.5 (M+H)⁺.

### Synthesis of 4-(4-((4′-chloro-4-(methoxycarbonyl)-[1,1′-biphenyl]-2-yl)methyl)piperazin-1-yl)benzoic Acid (Intermediate LS)

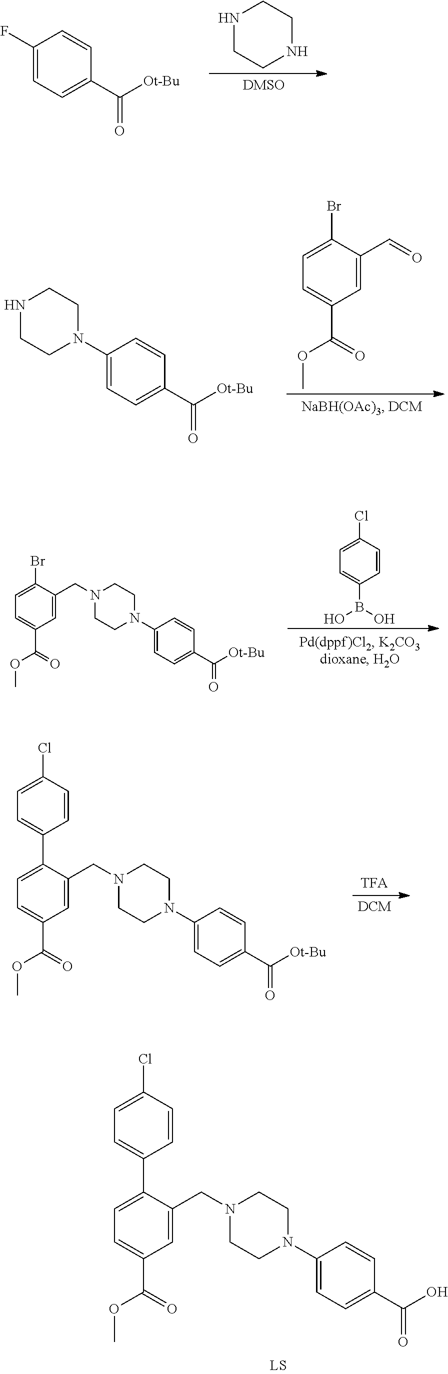

### Step 1—Tert-butyl 4-(piperazin-1-yl)benzoate

To a solution of piperazine (2.90 g, 33.6 mmol, CAS #110-85-0) in DMSO (40 mL) was added tert-butyl 4-fluorobenzoate (3 g, 15.3 mmol, CAS #58656-98-7). The mixture was stirred at 120° C. for 48 hrs. On completion, water (80 mL) was added to the mixture which caused precipitation of the solid. Then the mixture was filtered and the filter cake was dried to give the title compound (2.52 g, 55% yield) as a brown solid. LC-MS (ESI⁺) m/z 262.9 (M+H)⁺.

### Step 2—Methyl 4-bromo-3-((4-(4-(tert-butoxycarbonyl)phenyl)piperazin-1-yl)methyl)benzoate

To a solution of methyl 4-bromo-3-formylbenzoate (2.45 g, 10.1 mmol, CAS #858124-35-3) in DCM (20 mL) was added NaBH(OAc)₃ (2.13 g, 10.1 mmol) and tert-butyl 4-(piperazin-1-yl)benzoate (2.4 g, 9.15 mmol). The mixture was stirred at 25° C. for 12 hrs. On completion, PE (40 mL) was added to the reaction mixture which caused precipitation of a solid. The mixture was filtered, then the filter cake was dried to give the title compound (2.14 g, 45% yield) as a brown solid. LC-MS (ESI⁺) m/z 491.0 (M+H)⁺.

### Step 3—Methyl 2-((4-(4-(tert-butoxycarbonyl)phenyl)piperazin-1-yl)methyl)-4′-chloro-[1,1′-biphenyl]-4-carboxylate

To a solution of methyl 4-bromo-3-((4-(4-(tert-butoxycarbonyl)phenyl)piperazin-1-yl)methyl)benzoate (2 g, 4.09 mmol) in dioxane (16 mL) and H₂O (4 mL) was added (4-chlorophenyl)boronic acid (959 mg, 6.13 mmol, CAS #1679-18-1), K₂CO₃ (1.69 g, 12.3 mmol) and Pd(dppf)Cl₂ (299 mg, 409 μmol). The mixture was then stirred at 55° C. for 2 hrs under N₂ atmosphere. On completion, the residue was diluted with H₂O (30 mL) and extracted with DCM (30 mL×3). Then the reaction mixture was concentrated under reduced pressure to remove solvent. The residue was purified by column chromatography (SiO₂, Petroleum ether/Ethyl acetate=10/1 to 8/1) to give the title compound (1.82 g, 83% yield) as a yellow solid. LC-MS (ESI⁺) m/z 521.3 (M+H)⁺.

### Step 4—4-(4-((4′-Chloro-4-(methoxycarbonyl)-[1,1′-biphenyl]-2-yl)methyl)piperazin-1-yl)benzoic Acid

To a solution of methyl 2-((4-(4-(tert-butoxycarbonyl)phenyl)piperazin-1-yl)methyl)-4′-chloro-[1,1′-biphenyl]-4-carboxylate (1.7 g, 3.26 mmol) in DCM (50 mL) was added TFA (7.81 g, 69 mmol, 5.07 mL). The mixture was then stirred at 20-30° C. for 12 hrs. On completion, the reaction mixture was concentrated under reduced pressure to remove the solvent. Then the crude product was purified by reversed-phase HPLC (0.1% FA condition) to give the title compound (1.47 g, 91% yield) as a white solid. LC-MS (ESI⁺) m/z 465.2 (M+H)⁺.

### Synthesis of (R)-4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-carboxylic Acid (Intermediate LT)

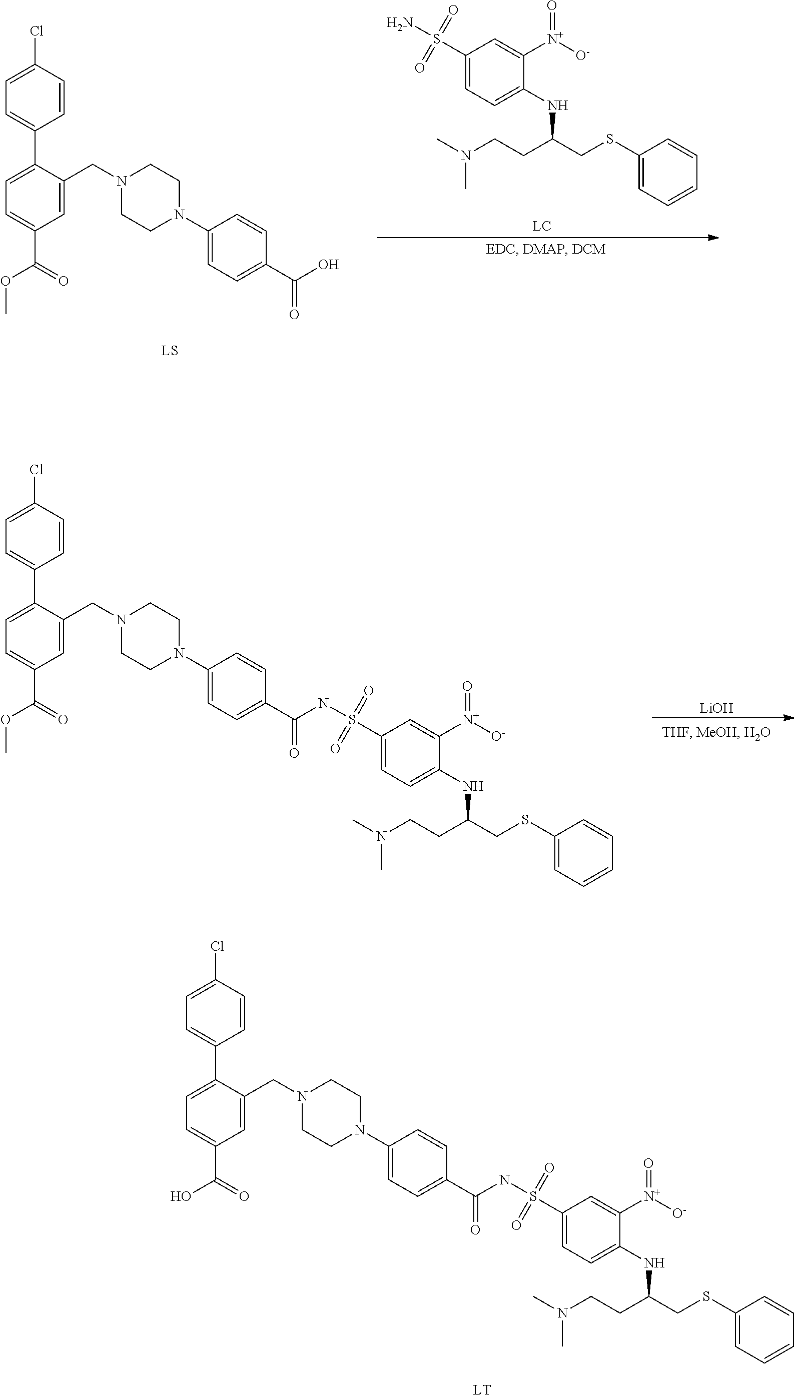

### Step 1—(R)-methyl 4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-carboxylate

To a solution of 4-(4-((4′-chloro-4-(methoxycarbonyl)-[1,1′-biphenyl]-2-yl)methyl)piperazin-1-yl)benzoic acid (300 mg, 645 μmol, Intermediate LS) in DCM (6 mL) was added EDC (200 mg, 1.29 mmol, 228 uL), (R)-4-((4-(dimethylamino)-1-(phenylthio)butan-2-yl)amino)-3-nitrobenzenesulfonamide (274 mg, 645 μmol, Intermediate LC) and DMAP (197 mg, 1.61 mmol). The mixture was then stirred at 30° C. for 12 hrs. On completion, the reaction mixture was concentrated under reduced pressure to remove solvent. Then the crude product was purified by reversed-phase HPLC (0.1% FA condition) to give the title compound (210 mg, 37% yield) as an orange solid. LC-MS (ESI⁺) m/z 871.3 (M+H)⁺.

### Step 2—(R)-4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-carboxylic Acid

To a solution of (R)-methyl 4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-carboxylate (100 mg, 115 μmol) in THF (3 mL), MeOH (3 mL) and H₂O (1.2 mL) was added LiOH·H₂O (24.1 mg, 574 μmol). Then the mixture was stirred at 50° C. for 12 hrs. On completion, the reaction mixture was concentrated under reduced pressure to remove solvent. Then the residue was diluted with H₂O (30 mL) and extracted with DCM (30 mL×3). The combined organic layers were washed with sat. NaCl (30 mL×3) and concentrated to give the title compound (90 mg, 90% yield) as an orange solid. LC-MS (ESI⁺) m/z 857.5 (M+H)⁺.

### Synthesis of (2S,4R)-1-((S)-2-(7-aminoheptanamido)-3,3-dimethylbutanoyl)-4-hydroxy-N—((S)-1-(4-(4-methylthiazol-5-yl)phenyl)ethyl)pyrrolidine-2-carboxamide (Intermediate LU)

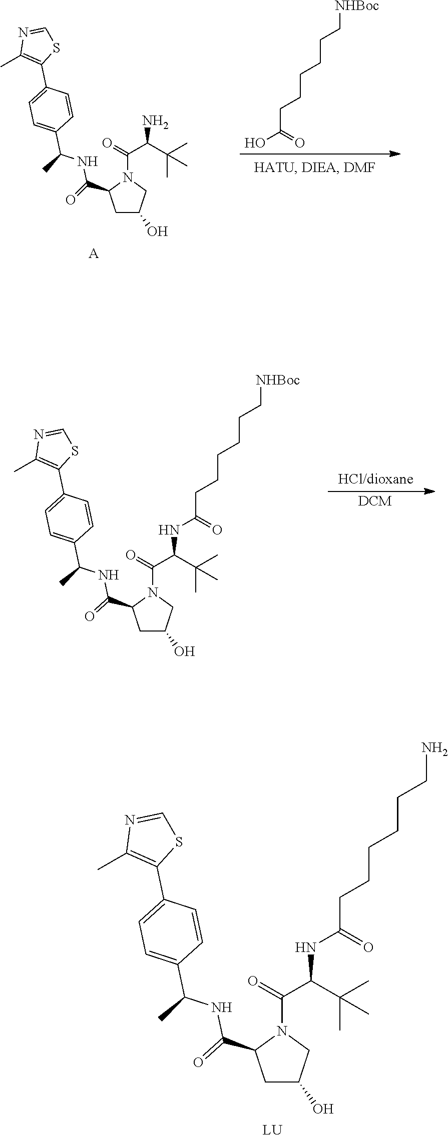

### Step 1—Tert-butyl (7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)carbamate

To a solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (362 mg, 815 μmol, Intermediate A) in DMF (4 mL) was added DIEA (1.05 g, 8.15 mmol, 1.42 mL), 7-(tert-butoxycarbonylamino)heptanoic acid (200 mg, 815 μmol, CAS #60142-89-4) and HATU (341 mg, 897 μmol). The mixture was stirred at 0° C. for 10 min. On completion, the reaction mixture was concentrated under reduced pressure to remove solvent. Then the residue was purified by RPLC (basic condition) to give the title compound (409 mg, 74% yield) as a white solid. LC-MS (ESI⁺) m/z 672.6 (M+H)⁺.

### Step 2—(2S,4R)-1-((S)-2-(7-aminoheptanamido)-3,3-dimethylbutanoyl)-4-hydroxy-N—((S)-1-(4-(4-methylthiazol-5-yl)phenyl)ethyl)pyrrolidine-2-carboxamide

To a solution of tert-butyl (7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptyl)carbamate (100 mg, 149 μmol) in DCM (2 mL) was added HCl/dioxane (4 M, 0.5 mL). The mixture was then stirred at 25° C. for 1 hr. On completion, the reaction mixture was concentrated under reduced pressure to remove solvent to give the title compound (60 mg) as a yellow solid. LC-MS (ESI⁺) m/z 572.3 (M+H)⁺.

### Synthesis of (2S,4R)-1-((S)-3,3-dimethyl-2-(8-oxooctanamido)butanoyl)-4-hydroxy-N—((S)-1-(4-(4-methylthiazol-5-yl)phenyl)ethyl)pyrrolidine-2-carboxamide (Intermediate LV)

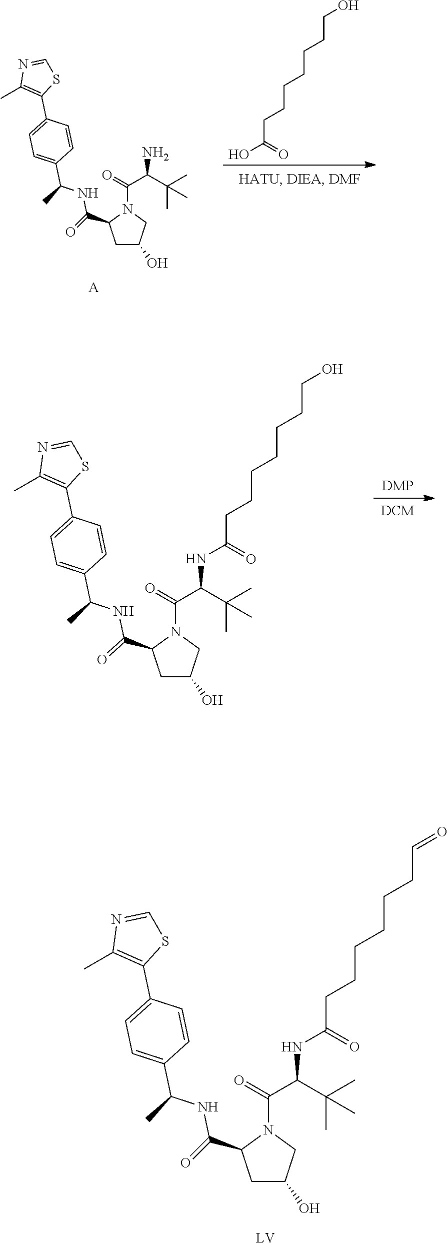

### Step 1—(2S,4R)-4-hydroxy-1-((S)-2-(8-hydroxyoctanamido)-3,3-dimethylbutanoyl)-N—((S)-1-(4-(4-methylthiazol-5-yl)phenyl)ethyl)pyrrolidine-2-carboxamide

To a solution of 8-hydroxyoctanoic acid (200 mg, 1.25 mmol, CAS #764-89-6) in DMF (4 mL) was added DIEA (1.61 g, 12.5 mmol), HATU (522 mg, 1.37 mmol) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (555 mg, 1.25 mmol, Intermediate A). The mixture was then stirred at 0° C. for 10 min. On completion, the reaction mixture was concentrated in vacuo to remove solvent. Then the crude residue was purified by reversed-phase HPLC (0.1% ammonium hydroxide) to give the title compound (341 mg, 46% yield) as a white solid. LC-MS (ESI⁺) m/z 587.3 (M+H)⁺.

### Step 2—(2S,4R)-1-((S)-3,3-dimethyl-2-(8-oxooctanamido)butanoyl)-4-hydroxy-N—((S)-1-(4-(4-methylthiazol-5-yl)phenyl)ethyl)pyrrolidine-2-carboxamide

To a solution of (2S,4R)-4-hydroxy-1-((S)-2-(8-hydroxyoctanamido)-3,3-dimethylbutanoyl)-N—((S)-1-(4-(4-methylthiazol-5-yl)phenyl)ethyl)pyrrolidine-2-carboxamide (100 mg, 170 μmol) in DCM (1 mL) was added DMP (108 mg, 256 μmol). Then the mixture was stirred at 25° C. for 30 min. On completion, the reaction mixture was diluted by water (2 mL) and extracted by dichloromethane (3×2 mL). The combined organic layers were washed by brine (6 mL) and dried over anhydrous sodium sulfate, filtered and the filtrate was concentrated in vacuo to give the title compound (90 mg) as a colorless oil. LC-MS (ESI⁺) m/z 585.5 (M+H)⁺.

### Synthesis of (R)-tert-butyl 4-(3-((2-nitro-4-sulfamoylphenyl)amino)-4-(phenylthio)butyl)piperazine-1-carboxylate (Intermediate LW)

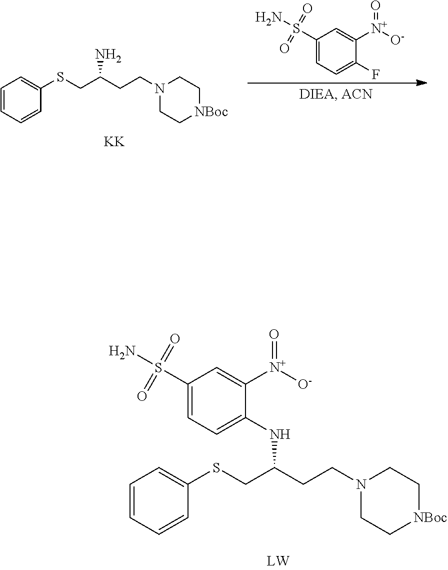 [SMILES: CC(C)(C)OC(=O)N1CCN(CC[C@@H](N)CSc2ccccc2)CC1 ; NS(=O)(=O)c1ccc(F)c([N+](=O)[O-])c1 ; "KK" ; CC(C)(C)OC(=O)N1CCN(CC[C@H](CSc2ccccc2)Nc2ccc(S(N)(=O)=O)cc2[N+](=O)[O-])CC1]

### Step 1—(R)-tert-butyl 4-(3-((2-nitro-4-sulfamoylphenyl)amino)-4-(phenylthio)butyl)piperazine-1-carboxylate (Intermediate LW)

To a solution of (R)-tert-butyl 4-(3-amino-4-(phenylthio)butyl)piperazine-1-carboxylate (400 mg, 972 μmol, FA, Intermediate KK) and 4-fluoro-3-nitrobenzenesulfonamide (256 mg, 1.17 mmol, CAS #406233-31-6) in ACN (10 mL) was added DIEA (628 mg, 4.86 mmol) at 25° C., then the mixture was stirred at 80° C. for 15 hrs. On completion, the reaction mixture was concentrated in vacuo to give the crude residue. The crude product was purified by reversed-phase HPLC (0.8 g/L ammonium bicarbonate) to give the title compound (195 mg, 35% yield) as a yellow solid. LC-MS (ESI⁺) m/z 565.8. (M+H)⁺.

### Synthesis of 4′-Chloro-[1,1′-biphenyl]-2-carbaldehyde (Intermediate LX)

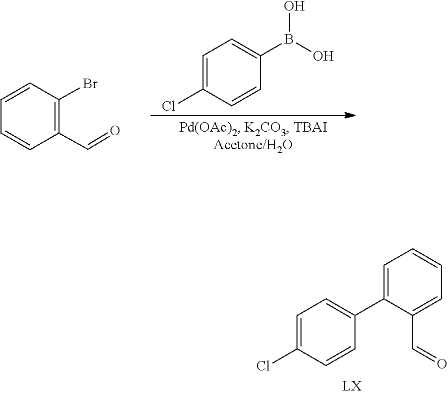 [SMILES: O=Cc1ccccc1Br ; OB(O)c1ccc(Cl)cc1 ; O=Cc1ccccc1-c1ccc(Cl)cc1]

### Step 1—4′-Chloro-[1,1′-biphenyl]-2-carbaldehyde (Intermediate LX)

A mixture of 2-bromobenzaldehyde (20 g, 108 mmol, 12.5 mL, CAS #6630-33-7), (4-chlorophenyl)boronic acid (16.9 g, 108 mmol, CAS #1679-18-1), tetra-(n-butyl) ammonium iodide (399 mg, 1.08 mmol, CAS #311-28-4), K₂CO₃ (44.8 g, 324 mmol) and Pd(OAc)₂ (1.21 g, 5.40 mmol, CAS #3375-31-3) in acetone (10 mL) and H₂O (10 mL) was degassed and purged with N₂ three times. Then the mixture was stirred at 40° C. for 1 hr under N₂ atmosphere. On completion, the reaction mixture was quenched with water (20 mL) and extracted by Ethyl acetate (2×50 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=100/1 to 20/1) to give the title compound (18 g, 77% yield) as a white solid. LC-MS (ESI⁺) m/z 217.2 (M+H)⁺.

### Synthesis of 4-(4-((4′-Chloro-[1,1′-biphenyl]-2-yl)methyl)piperazin-1-yl)benzoic Acid (Intermediate LY)

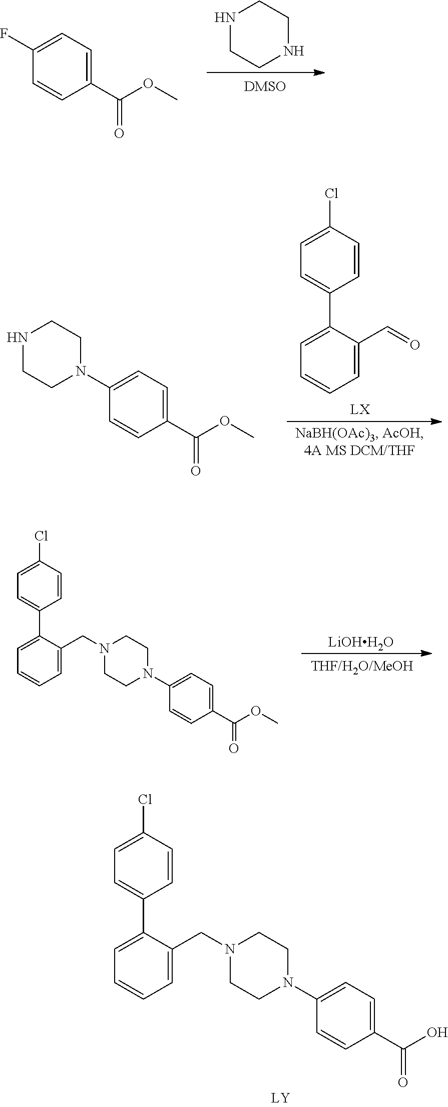

### Step 1—Methyl 4-(piperazin-1-yl)benzoate

A solution of methyl 4-fluorobenzoate (25 g, 162 mmol, CAS #403-33-8) and piperazine (16 g, 194 mmol, CAS #110-85-0) in DMSO (250 mL) was stirred at 125° C. for 12 hrs. On completion, the reaction mixture was diluted with water (1000 mL) and extracted by ethyl acetate (3×1000 mL). Then sat. NaHCO₃ was added into the solution until the pH was 8. Then the mixture was filtered and the filter cake was dried in vacuo to give the title compound (4.7 g) as a white solid. LC-MS (ESI⁺) m/z 221.1 (M+1)⁺.

### Step 2—Methyl 4-(4-((4′-chloro-[1,1′-biphenyl]-2-yl)methyl)piperazin-1-yl)benzoate

To a solution of methyl 4-(piperazin-1-yl)benzoate (5 g, 22.7 mmol) and 4′-chloro-[1,1′-biphenyl]-2-carbaldehyde (5.41 g, 24.9 mmol, Intermediate LX) in THF (40 mL) and DCM (40 mL) was added AcOH (1.36 g, 22.7 mmol), NaBH(OAc)₃ (19.2 g, 90.8 mmol) and 4 Å molecular sieves (3 g) at 25° C. Then the mixture was stirred at 40° C. for 16 hrs. On completion, the reaction mixture was quenched with water (50 mL) and extracted by ethyl acetate (3×30 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=5/1 to 3/1) to give the title compound (4.7 g, 48% yield) as a white solid. LC-MS (ESI⁺) m/z 421.4 (M+1)⁺.

### Step 3—4-(4-((4′-Chloro-[1,1′-biphenyl]-2-yl)methyl)piperazin-1-yl)benzoic Acid

To a solution of methyl 4-(4-((4′-chloro-[1,1′-biphenyl]-2-yl)methyl)piperazin-1-yl)benzoate (1 g, 2.38 mmol) in H₂O (2 mL), THF (8 mL) and MeOH (2 mL) was added LiOH·H₂O (996 mg, 23.7 mmol) at 25° C. Then the mixture was stirred at 50° C. for 12 hrs. On completion, the reaction mixture was added HCl (1N) until the pH was 5, then diluted by water (10 mL) and extracted by ethyl acetate (3×15 mL). The combined organic layers were dried over anhydrous sodium sulfate, filtered and the filtrate was concentrated in vacuo to give the title compound (1 g) as a white solid. LC-MS (ESI⁺) m/z 407.4 (M+1)⁺.

### Synthesis of (R)-4-(4-((4′-chloro-[1,1′-biphenyl]-2-yl)methyl)piperazin-1-yl)-N-((3-nitro-4-((1-(phenylthio)-4-(piperazin-1-yl)butan-2-yl)amino)phenyl)sulfonyl)benzamide (Intermediate LZ)

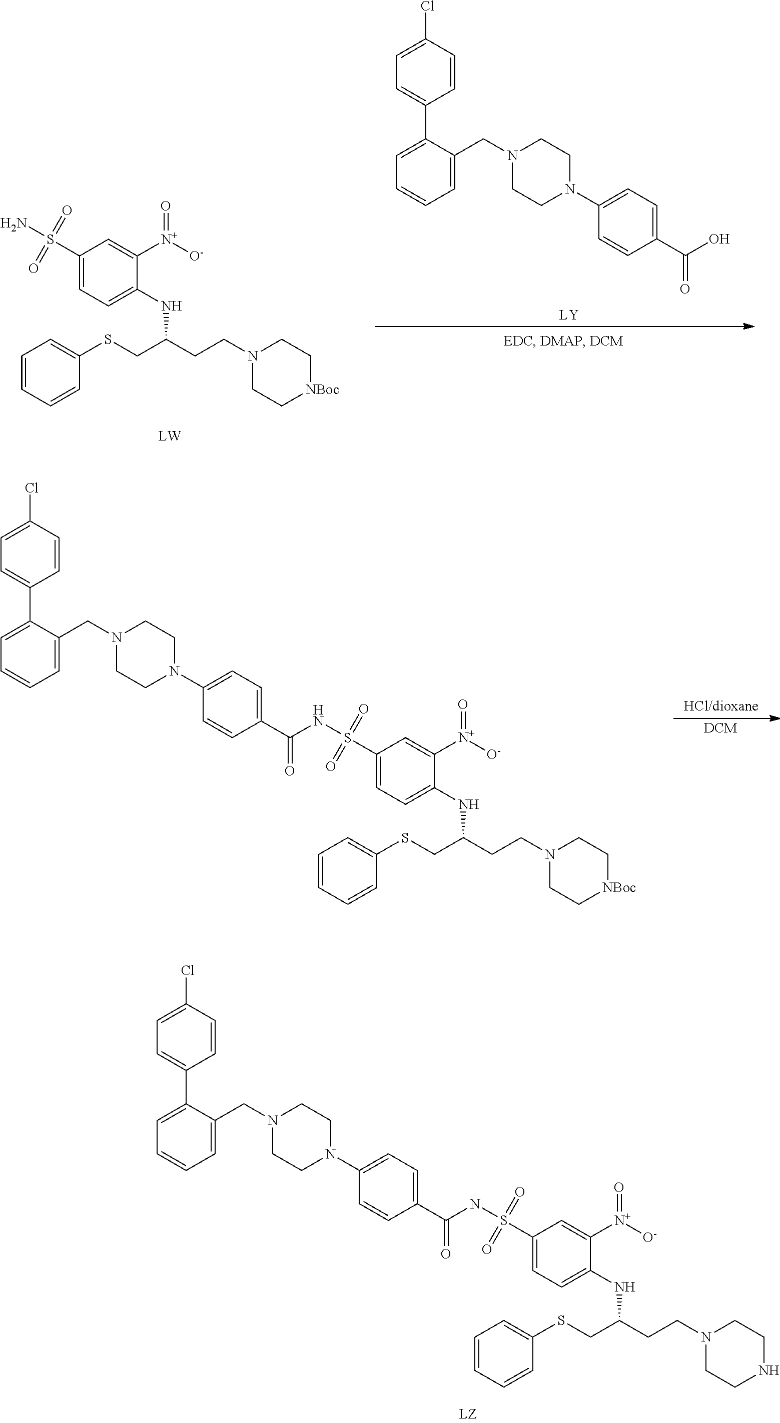

### Step 1—(R)-tert-butyl 4-(3-((4-(N-(4-(4-((4′-chloro-[1,1′-biphenyl]-2-yl)methyl)piperazin-1-yl)benzoyl)sulfamoyl)-2-nitrophenyl)amino)-4-(phenylthio)butyl)piperazine-1-carboxylate

To a solution of 4-(4-((4′-chloro-[1,1′-biphenyl]-2-yl)methyl)piperazin-1-yl)benzoic acid (184 mg, 453 μmol, Intermediate LY) in DCM (1 mL) was added EDC (93.9 mg, 605 μmol) and DMAP (92.4 mg, 756 μmol). Then (R)-tert-butyl 4-(3-((2-nitro-4-sulfamoylphenyl)amino)-4-(phenylthio)butyl)piperazine-1-carboxylate (185 mg, 302 μmol, FA, Intermediate LW) was added at 25° C. and the mixture was stirred at 30° C. for 4 hrs. On completion, the reaction mixture was concentrated in vacuo to give the crude residue. The crude product was purified by reversed-phase HPLC (0.1% FA condition) to give the title compound (200 mg, 64% yield, FA) as a yellow solid. LC-MS (ESI⁺) m/z 954.0. (M+H)⁺.

### Step 2—(R)-4-(4-((4′-chloro-[1,1′-biphenyl]-2-yl)methyl)piperazin-1-yl)-N-((3-nitro-4-((1-(phenylthio)-4-(piperazin-1-yl)butan-2-yl)amino)phenyl)sulfonyl)benzamide

To a solution of tert-butyl 4-[(3R)-3-[4-[[4-[4-[[2-(4-chlorophenyl)phenyl]methyl]piperazin-1-yl]benzoyl]sulfamoyl]-2-nitro-anilino]-4-phenylsulfanyl-butyl]piperazine-1-carboxylate (190 mg, 190 μmol, FA) in DCM (4 mL) was added HCl/dioxane (4 M, 0.8 mL) at 25° C., then the mixture was stirred at 25° C. for 1 hr. On completion, the reaction mixture was concentrated in vacuo to give the title compound (220 mg, HCl) as a yellow solid. LC-MS (ESI⁺) m/z 854.5. (M+H)⁺.

### Synthesis of (2S,4R)-1-((S)-3,3-dimethyl-2-(7-oxoheptanamido)butanoyl)-4-hydroxy-N—((S)-1-(4-(4-methylthiazol-5-yl)phenyl)ethyl)pyrrolidine-2-carboxamide (Intermediate MA)

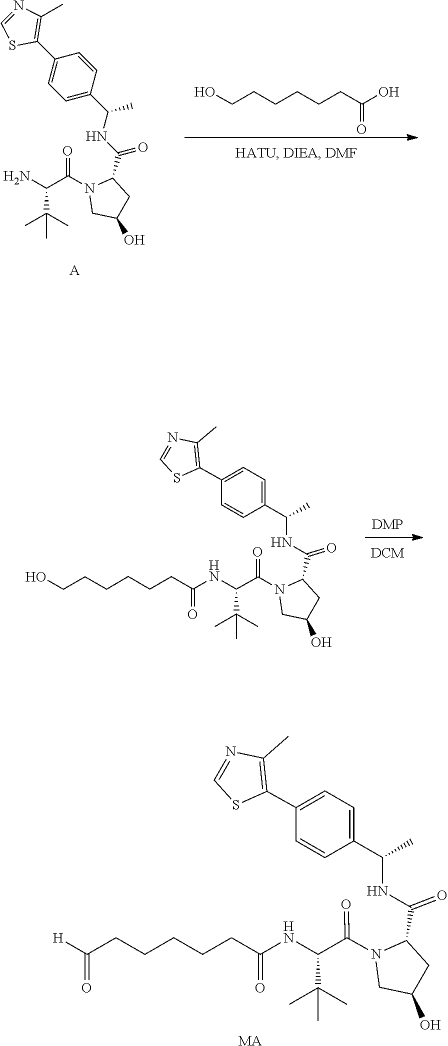

### Step 1—(2S,4R)-4-hydroxy-1-((S)-2-(7-hydroxyheptanamido)-3,3-dimethylbutanoyl)-N—((S)-1-(4-(4-methylthiazol-5-yl)phenyl)ethyl)pyrrolidine-2-carboxamide

To a solution of 7-hydroxyheptanoic acid (247 mg, 1.69 mmol, CAS #3710-42-7) in DMF (8 mL) was added DIEA (1.45 g, 11.3 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (500 mg, 1.12 mmol, Intermediate A) and HATU (470 mg, 1.24 mmol). The mixture was then stirred at 0° C. for 10 min. On completion, the reaction mixture was concentrated in vacuo to remove the solvent. Then the crude residue was purified by reversed-phase HPLC (0.1% FA condition) to give the title compound (230 mg, 36% yield, FA) as a white solid. LC-MS (ESI⁺) m/z 573.4 (M+H)⁺.

### Step 2—(2S,4R)-1-((S)-3,3-dimethyl-2-(7-oxoheptanamido)butanoyl)-4-hydroxy-N—((S)-1-(4-(4-methylthiazol-5-yl)phenyl)ethyl)pyrrolidine-2-carboxamide

To a solution of (2S,4R)-4-hydroxy-1-((S)-2-(7-hydroxyheptanamido)-3,3-dimethylbutanoyl)-N—((S)-1-(4-(4-methylthiazol-5-yl)phenyl)ethyl)pyrrolidine-2-carboxamide (100 mg, 175 μmol) in DCM (1 mL) was added DMP (111 mg, 262 μmol). The mixture was then stirred at 25° C. for 30 min. On completion, the reaction mixture was concentrated in vacuo to remove the solvent, then the reaction mixture was diluted by water (3 mL) and extracted by dichloromethane (3×3 mL). The combined organic layers were washed by NaHCO₃ (3 mL) and dried over anhydrous sodium sulfate, filtered and the filtrate was concentrated in vacuo to give the title compound (90 mg) as a white oil. LC-MS (ESI⁺) m/z 571.5 (M+H)⁺.

### Synthesis of (R)-tert-butyl (3-amino-4-(phenylthio)butyl)(methyl)carbamate (Intermediate MB)

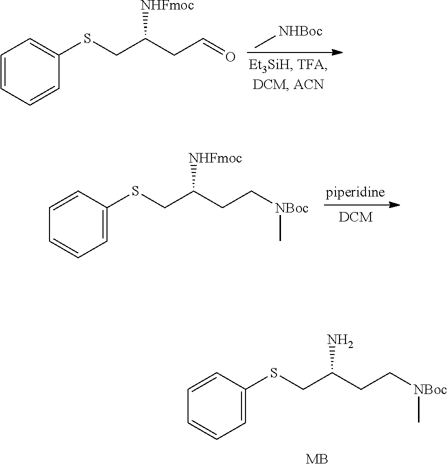

### Step 1—(R)—N-((4-((1-(phenylthio)-4-(piperazin-1-yl)butan-2-yl)amino)-3-((trifluoromethyl)sulfonyl)phenyl)sulfonyl)-4-(2-azaspiro[3,3]heptan-2-yl)benzamide

To a solution of (R)-(9H-fluoren-9-yl)methyl (4-oxo-1-(phenylthio)butan-2-yl)carbamate (3.5 g, 8.38 mmol, synthesized via Steps 1-3 of Intermediate KK) in DCM (20 mL) and ACN (60 mL) was added tert-butyl N-methylcarbamate (2.20 g, 16.8 mmol, CAS #16066-84-5), Et₃SiH (4.87 g, 41.9 mmol, 6.69 mL) and TFA (2.87 g, 25.2 mmol, 1.86 mL) at 0° C. Then the reaction was stirred at 25° C. for 12 hrs. On completion, the reaction mixture was quenched with H₂O (20 mL) at 25° C., and then extracted with DCM (3×50 mL). The combined organic layers were washed with brine (2×100 mL), dried over anhydrous sodium sulfate, filtered and concentrated under reduced pressure to give a residue. The residue was purified by column chromatography (SiO₂, Petroleum ether/Ethyl acetate=1/0 to 5/1) to give the title compound (2 g, 43% yield) as a yellow solid. LC-MS (ESI⁺) m/z 533.1. (M+H)⁺.

### Step 2—(R)-tert-butyl (3-amino-4-(phenylthio)butyl)(methyl)carbamate

To a solution of (R)-tert-butyl (3-((((9H-fluoren-9-yl)methoxy) carbonyl)amino)-4-(phenylthio)butyl)(methyl)carbamate (2 g, 3.75 mmol) in DCM (15 mL) was added piperidine (3.84 g, 45.1 mmol, 4.45 mL). The reaction was stirred at 25° C. for 2 hrs. On completion, the reaction mixture was concentrated under reduced pressure to give a residue. The residue was purified by reversed-phase HPLC (0.1% FA condition) to give the title compound (900 mg, 75% yield) as a yellow oil. LC-MS (ESI⁺) m/z 311.1. (M+H)⁺.

### Synthesis of (R)-tert-butyl methyl (3-((2-nitro-4-sulfamoylphenyl)amino)-4-(phenylthio)butyl)carbamate (Intermediate MC)

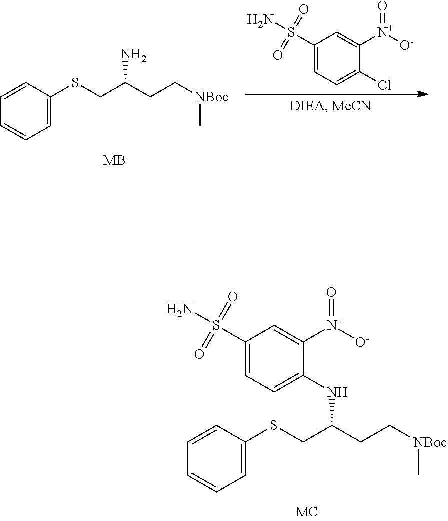 [SMILES: CN(CC[C@@H](N)CSc1ccccc1)C(=O)OC(C)(C)C ; NS(=O)(=O)c1ccc(Cl)c([N+](=O)[O-])c1 ; CN(CC[C@H](CSc1ccccc1)Nc1ccc(S(N)(=O)=O)cc1[N+](=O)[O-])C(=O)OC(C)(C)C]

To a solution of tert-butyl (R)-tert-butyl (3-amino-4-(phenylthio)butyl)(methyl)carbamate (500 mg, 1.61 mmol, Intermediate MB) in ACN (10 mL) was added DIEA (1.04 g, 8.05 mmol, 1.40 mL) and 4-chloro-3-nitro-benzenesulfonamide (572 mg, 2.42 mmol, CAS #97-09-6). The mixture was stirred at 80° C. for 12 hrs. On completion, the reaction mixture was concentrated under reduced pressure to give a residue. The residue was purified by column chromatography (SiO₂, Petroleum ether/Ethyl acetate=10/1 to 2/1 to DCM:MeOH=20:1) to give the title compound (300 mg, 22% yield) as a yellow oil. LC-MS (ESI⁺) m/z 411.1. (M+H)⁺.

### Synthesis of (R)-4-(4-((4′-chloro-[1,1′-biphenyl]-2-yl)methyl)piperazin-1-yl)-N-((4-((4-(methylamino)-1-(phenylthio)butan-2-yl)amino)-3-nitrophenyl)sulfonyl)benzamide (Intermediate MD)

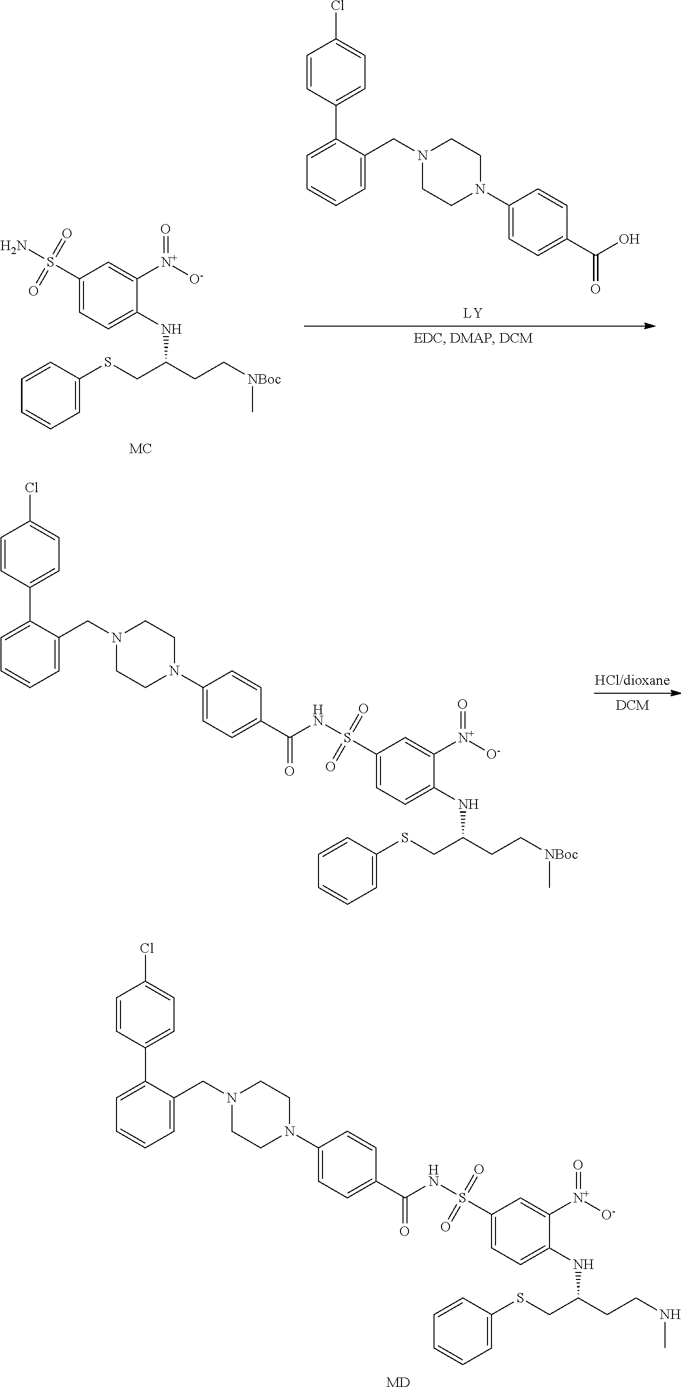

### Step 1—(R)-tert-butyl (3-((4-(N-(4-(4-((4′-chloro-[1,1′-biphenyl]-2-yl)methyl)piperazin-1-yl)benzoyl)sulfamoyl)-2-nitrophenyl)amino)-4-(phenylthio)butyl)(methyl)carbamate

To a solution of 4-(4-((4′-chloro-[1,1′-biphenyl]-2-yl)methyl)piperazin-1-yl)benzoic acid (254 mg, 625 μmol, Intermediate LY) in DCM (6 mL) was added DMAP (69.4 mg, 568 μmol), CMPI (218 mg, 852 μmol), TEA (172 mg, 1.70 mmol, 237 uL) and (R)-tert-butyl methyl (3-((2-nitro-4-sulfamoylphenyl)amino)-4-(phenylthio)butyl)carbamate (290 mg, 568 μmol, Intermediate MC). The reaction was then stirred at 25° C. for 2 hrs. On completion, the reaction mixture was concentrated under reduced pressure to give a residue. The residue was purified by reversed-phase HPLC (0.8 g/L ammonium bicarbonate condition) to give the title compound (140 mg, 26% yield) as a yellow solid. LC-MS (ESI⁺) m/z 899.4. (M+H)⁺.

### Step 2—(R)-4-(4-((4′-chloro-[1,1′-biphenyl]-2-yl)methyl)piperazin-1-yl)-N-((4-((4-(methylamino)-1-(phenylthio)butan-2-yl)amino)-3-nitrophenyl)sulfonyl)benzamide

To a solution of (R)-tert-butyl (3-((4-(N-(4-(4-((4′-chloro-[1,1′-biphenyl]-2-yl)methyl)piperazin-1-yl)benzoyl)sulfamoyl)-2-nitrophenyl)amino)-4-(phenylthio)butyl)(methyl)carbamate (80 mg, 88.9 μmol) in DCM (2 mL) was added HCl/dioxane (4 M, 0.5 mL). The reaction was stirred at 25° C. for 1 hr. On completion, the reaction mixture was concentrated under reduced pressure to give the title compound (80 mg, HCl) as a yellow solid. LC-MS (ESI⁺) m/z 799.5. (M+H)⁺.

### Synthesis of Methyl (R)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoate (Intermediate ME) and methyl(S)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoate (Intermediate MF)

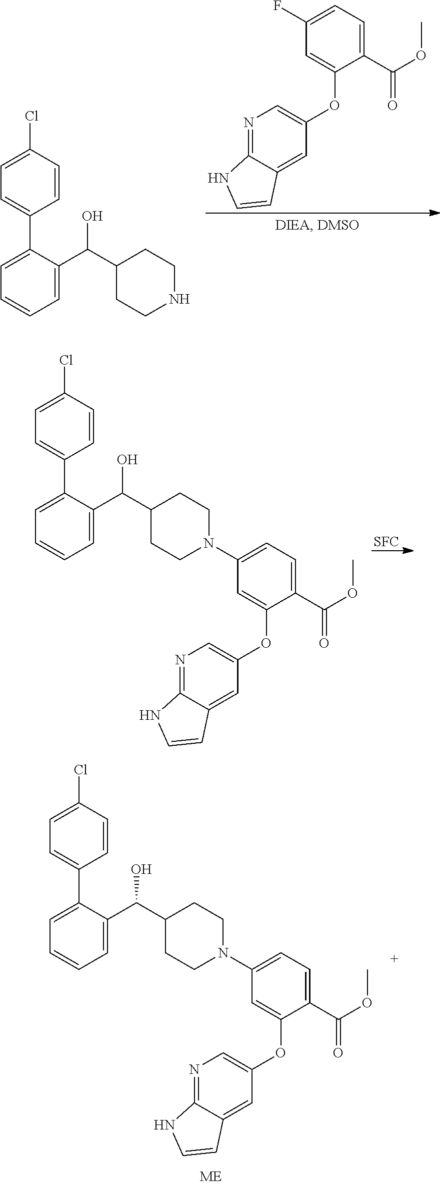

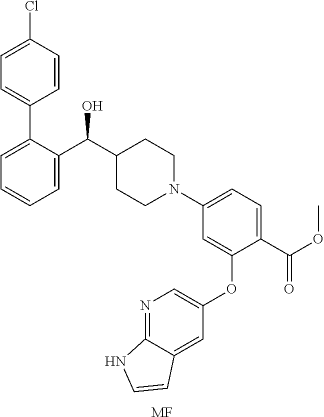

### Step 1—Methyl 2-((1H-pyrrolo[2,3-b]pyridin-5-yl)oxy)-4-(4-((4′-chloro-[1,1′-biphenyl]-2-yl)(hydroxy)methyl)piperidin-1-yl)benzoate

To a solution of (4′-chloro-[1,1′-biphenyl]-2-yl)(piperidin-4-yl) methanol (1.3 g, 4.31 mmol, synthesized via Step 1 of Intermediate KM) and methyl 2-((1H-pyrrolo[2,3-b]pyridin-5-yl)oxy)-4-fluorobenzoate (1.85 g, 6.46 mmol, CAS #1235865-75-4) in DMSO (50 mL) was added DIEA (8.35 g, 64.6 mmol, 11.3 mL). The mixture was then stirred at 120° C. for 12 hrs. On completion, the reaction mixture was diluted with water (50 mL) and extracted by ethyl acetate (3×50 mL). The combined organic layers were washed by brine (450 mL) and dried over anhydrous sodium sulfate, filtered and the filtrate was concentrated in vacuo to give the crude residue. The residue was purified by column chromatography (SiO₂, Petroleum ether:Ethyl acetate=1/0 to 2/1) to give the title compound (740 mg, 26% yield) as a white solid. LC-MS (ESI⁺) m/z 568.1 (M+H)⁺.

### Step 2—Methyl (R)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoate and methyl(S)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoate

Methyl 2-((1H-pyrrolo[2,3-b]pyridin-5-yl)oxy)-4-(4-((4′-chloro-[1,1′-biphenyl]-2-yl)(hydroxy)methyl)piperidin-1-yl)benzoate was purified by SFC (column: DAICEL CHIRALCEL OD (250 mm×30 mm×10 um); mobile phase: [0.1% NH₃H₂O IPA]; B %: 40%-40%, A5; 22 min) to give methyl (R)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoate (295 mg, 512 μmol, 39% yield) and methyl(S)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoate 305 mg, 530 μmol, 41% yield as white solids. LC-MS (ESI⁺) m/z 568.3 (M+H)⁺. The absolute stereochemistry of the enantiomers was assigned arbitrarily.

### Synthesis of (R)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoic Acid (Intermediate MG)

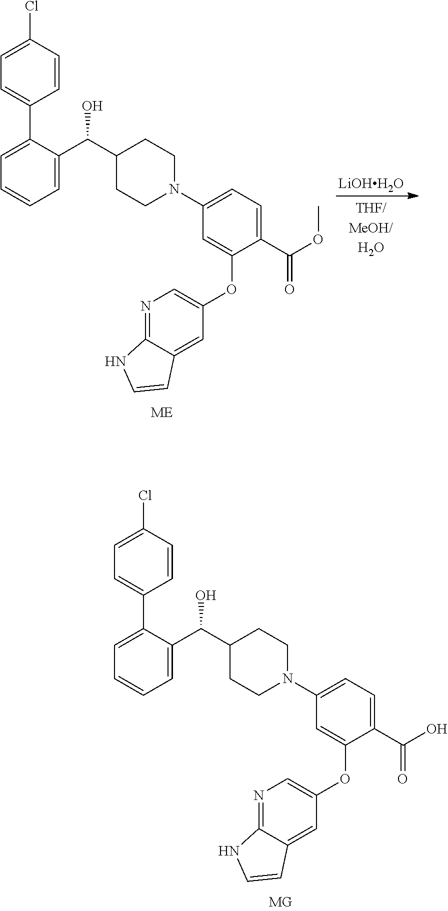

To a solution of methyl (R)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoate (295 mg, 519 μmol, Intermediate ME) in THF (4 mL), MeOH (1 mL) and H₂O (1 mL) was added LiOH·H₂O (109 mg, 2.60 mmol). The mixture was stirred at 50° C. for 12 hrs. On completion, HCl (1 M) was added to the mixture until the pH was 6.0 and extracted by ethyl acetate (2×25 mL). The combined organic layers were dried over anhydrous sodium sulfate, filtered and the filtrate was concentrated in vacuo to give the title compound (260 mg) as a yellow solid. LC-MS (ESI⁺) m/z 554.1 (M+H)⁺.

### Synthesis of (R)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3-(piperazin-1-yl)propyl)amino)-3-((trifluoromethyl)sulfonyl)phenyl)sulfonyl)benzamide (Intermediate MH)

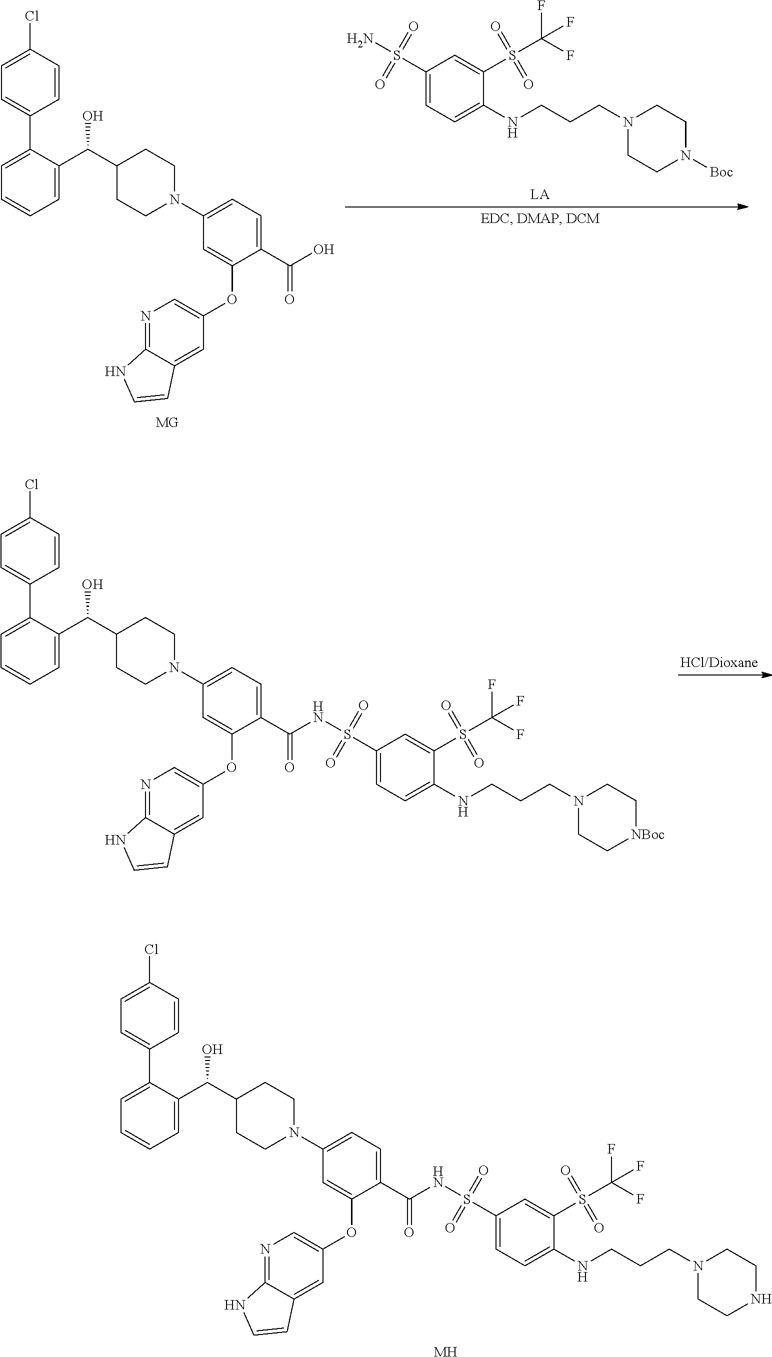 [SMILES: O=C(O)c1ccc(N2CCC([C@@H](O)c3ccccc3-c3ccc(Cl)cc3)CC2)cc1Oc1cnc2[nH]ccc2c1 ; CC(C)(C)OC(=O)N1CCN(CCCNc2ccc(S(N)(=O)=O)cc2S(=O)(=O)C(F)(F)F)CC1 ; CC(C)(C)OC(=O)N1CCN(CCCNc2ccc(S(=O)(=O)NC(=O)c3ccc(N4CCC([C@@H](O)c5ccccc5-c5ccc(Cl)cc5)CC4)cc3Oc3cnc4[nH]ccc4c3)cc2S(=O)(=O)C(F)(F)F)CC1 ; O=C(NS(=O)(=O)c1ccc(NCCCN2CCNCC2)c(S(=O)(=O)C(F)(F)F)c1)c1ccc(N2CCC([C@@H](O)c3ccccc3-c3ccc(Cl)cc3)CC2)cc1Oc1cnc2[nH]ccc2c1 ; "MH"]

### Step 1—Tert-butyl (R)-4-(3-((4-(N-(2-((1H-pyrrolo[2,3-b]pyridin-5-yl)oxy)-4-(4-((4′-chloro-[1,1′-biphenyl]-2-yl)(hydroxy)methyl)piperidin-1-yl)benzoyl)sulfamoyl)-2-((trifluoromethyl)sulfonyl)phenyl)amino)propyl)piperazine-1-carboxylate

To a solution of (R)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoic acid (200 mg, 361 μmol, Intermediate MG) in DCM (4 mL) was added EDC (280 mg, 1.80 mmol, 319.50 uL), tert-butyl 4-(3-((4-sulfamoyl-2-((trifluoromethyl)sulfonyl)phenyl)amino)propyl)piperazine-1-carboxylate (192 mg, 361 μmol, Intermediate LA) and DMAP (221 mg, 1.80 mmol). The mixture was then stirred at 25° C. for 12 hrs. On completion, the reaction mixture was concentrated under reduced pressure and purified by reverse phase (FA condition) to give the title compound (100 mg, 24% yield, FA) as a yellow solid. LC-MS (ESI⁺) m/z 1066.4 (M+H)⁺.

### Step 2—(R)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3-(piperazin-1-yl)propyl)amino)-3-((trifluoromethyl)sulfonyl)phenyl)sulfonyl)benzamide

To a solution of tert-butyl (R)-4-(3-((4-(N-(2-((1H-pyrrolo[2,3-b]pyridin-5-yl)oxy)-4-(4-((4′-chloro-[1,1′-biphenyl]-2-yl)(hydroxy)methyl)piperidin-1-yl)benzoyl)sulfamoyl)-2-((trifluoromethyl)sulfonyl)phenyl)amino)propyl)piperazine-1-carboxylate (100 mg, 93.8 μmol) in DCM (2 mL) was added HCl/Dioxane (4 M, 70.3 uL). The mixture was stirred at 25° C. for 0.5 hr. On completion, the reaction mixture was concentrated in vacuo to give the title compound (80 mg) as a yellow solid. LC-MS (ESI⁺) m/z 996.7 (M+H)⁺.

### Synthesis of (S)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoic Acid (Intermediate MI)

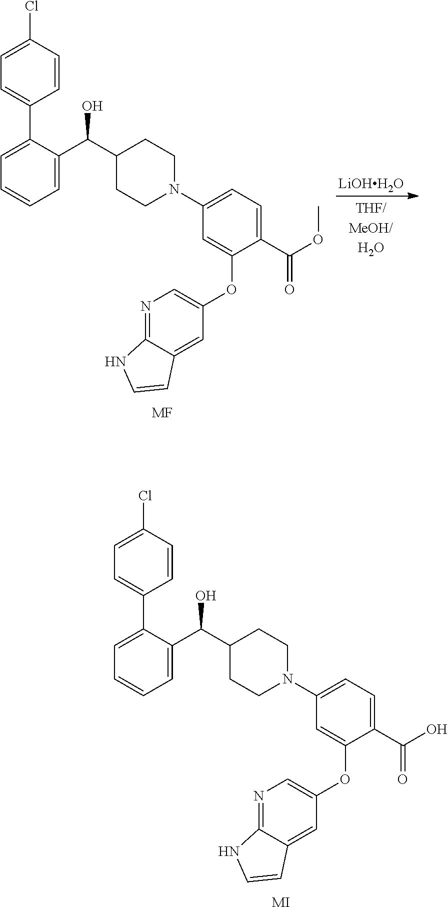

### Step 1—(S)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoic Acid (Intermediate MI)

To a solution of methyl 4-[4-[(S)-[2-(4-chlorophenyl)phenyl]-hydroxy-methyl]-1-piperidyl]-2-(1H-pyrrolo[2,3-b]pyridin-5-yloxy)benzoate (305 mg, 537 μmol) in THF (4 mL), MeOH (1 mL) and H₂O (1 mL) was added LiOH·H₂O (113 mg, 2.68 mmol). The mixture was stirred at 50° C. for 12 hrs. On completion, the reaction mixture was quenched with H₂O (5 mL) and extracted with EA (5 mL×3). The combined organic layers were dried over Na₂SO₄, filtered and the filtrate was concentrated to give the title product (260 mg) as yellow a solid. LC-MS (ESI⁺) m/z 554.1 (M+H)⁺.

### Synthesis of (S)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3-(piperazin-1-yl)propyl)amino)-3-((trifluoromethyl)sulfonyl)phenyl)sulfonyl)benzamide (Intermediate MJ)

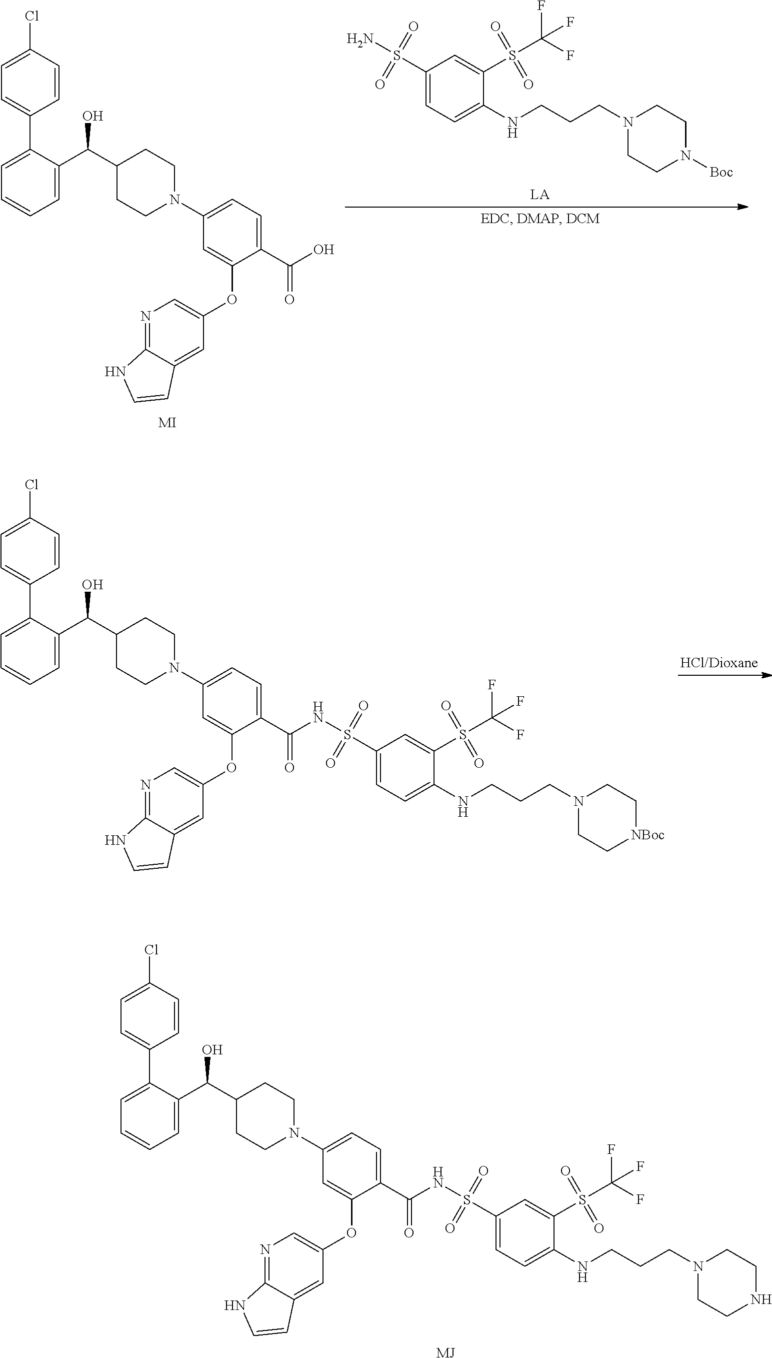

### Step 1—Tert-butyl 4-[3-[4-[[4-[4-[(S)-[2-(4-chlorophenyl)phenyl]-hydroxy-methyl]-1-piperidyl]-2-(1H-pyrrolo[2,3-b]pyridin-5-yloxy)benzoyl]sulfamoyl]-2-(trifluoromethylsulfonyl)anilino]propyl]piperazine-1-carboxylate

To a solution of 4-[4-[(S)-[2-(4-chlorophenyl)phenyl]-hydroxy-methyl]-1-piperidyl]-2-(1H-pyrrolo[2,3-b]pyridin-5-yloxy)benzoic acid (200 mg, 360 μmol, Intermediate MI) in DCM (4 mL) was added EDC (280.20 mg, 1.80 mmol), tert-butyl 4-[3-[4-sulfamoyl-2-(trifluoromethylsulfonyl)anilino]propyl]piperazine-1-carboxylate (191.53 mg, 360.99 μmol, Intermediate LA) and DMAP (220.51 mg, 1.80 mmol). The mixture was then stirred at 25° C. for 12 hrs. On completion, the reaction was concentrated directly and the residue was purified by reversed phase (0.1% FA) to give the title compound (100 mg, 25% yield) as a yellow solid. LC-MS (ESI⁺) m/z 1066.4 (M+H)⁺.

### Step 2—(S)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3-(piperazin-1-yl)propyl)amino)-3-((trifluoromethyl)sulfonyl)phenyl)sulfonyl)benzamide

To a solution of tert-butyl 4-[3-[4-[[4-[4-[(S)-[2-(4-chlorophenyl)phenyl]-hydroxy-methyl]-1-piperidyl]-2-(1H-pyrrolo[2,3-b]pyridin-5-yloxy)benzoyl]sulfamoyl]-2-(trifluoromethylsulfonyl) anilino]propyl]piperazine-1-carboxylate (100 mg, 93.7 μmol) in DCM (2 mL) was added HCl/Dioxane (4 M, 70.3 uL). The mixture was then stirred at 25° C. for 0.5 hrs. On completion, the reaction was concentrated to give the title compound (80 mg, HCl salt) as a yellow solid. LC-MS (ESI⁺) m/z 966.7 (M+H)⁺.

### Synthesis of 6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoic Acid (Intermediate MK)

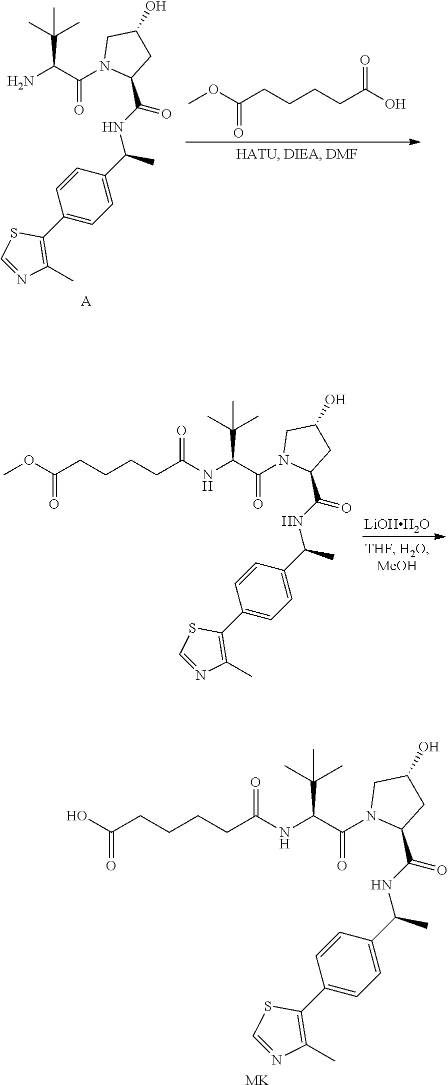

### Step 1—Methyl 6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoate

A solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (500 mg, 1.12 mmol, Intermediate A), 6-methoxy-6-oxohexanoic acid (215 mg, 1.34 mmol, 199 uL, CAS #627-91-8), HATU (511 mg, 1.34 mmol), and DIEA (434 mg, 3.36 mmol, 585 uL) in DMF (5 mL) was stirred at 25° C. for 12 hrs. On completion, the reaction mixture was quenched with water (10 mL) and extracted with ethyl acetate (2×10 mL). The combined organic layers were washed by brine (10 mL) and dried over anhydrous sodium sulfate, filtered and the filtrate was concentrated in vacuo to give the title compound (600 mg) as a yellow solid. LC-MS (ESI⁺) m/z 587.3 (M+1)⁺.

### Step 2—6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoic Acid

A solution of methyl 6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoate (200 mg, 340.87 μmol) and LiOH·H₂O (71.5 mg, 1.70 mmol) in THF (0.5 mL), H₂O (0.5 mL), MeOH (0.5 mL) was stirred at 25° C. for 12 hrs. On completion, the reaction mixture was quenched with water (10 mL) and extracted with ethyl acetate (2×10 mL). The combined organic layers were washed with brine (10 mL), dried over anhydrous sodium sulfate, filtered and the filtrate was concentrated in vacuo to give the title compound (600 mg) as a yellow solid. LC-MS (ESI⁺) m/z 587.3 (M+1)⁺.

### Synthesis of (2S,4R)-1-((S)-3,3-dimethyl-2-(6-oxo-6-(4-oxopiperidin-1-yl) hexanamido)butanoyl)-4-hydroxy-N—((S)-1-(4-(4-methylthiazol-5-yl)phenyl)ethyl)pyrrolidine-2-carboxamide (Intermediate ML)

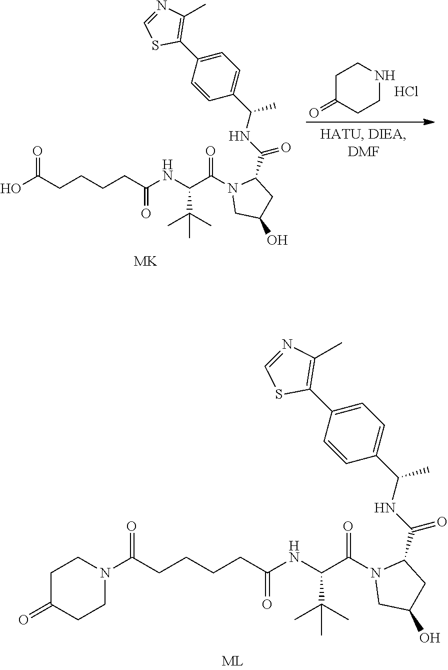

### Step 1—(2S,4R)-1-((S)-3,3-dimethyl-2-(6-oxo-6-(4-oxopiperidin-1-yl) hexanamido)butanoyl)-4-hydroxy-N—((S)-1-(4-(4-methylthiazol-5-yl)phenyl)ethyl)pyrrolidine-2-carboxamide (Intermediate ML)

To a solution of 6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoic acid (150 mg, 261 μmol, Intermediate MK) in DMF (1 mL) was added DIEA (169 mg, 1.31 mmol, 228 uL), HATU (119 mg, 314 μmol) and piperidin-4-one (51.9 mg, 382 μmol, HCl). The mixture was then stirred at 0-25° C. for 15 min. On completion, the reaction mixture was concentrated under reduced pressure to give a residue. The residue was purified by reversed-phase HPLC (0.8 g/L ammonium bicarbonate) to give the title compound (120 mg, 70% yield) as a white solid. LC-MS (ESI⁺) m/z 654.6 (M+H)⁺.

### Synthesis of 4-(4-((4-(((1R,4R)-5-(tert-butoxycarbonyl)-2,5-diazabicyclo[2.2.1]heptan-2-yl)methyl)-4′-chloro-[1,1′-biphenyl]-2-yl)methyl)piperazin-1-yl)benzoic Acid (Intermediate MM)

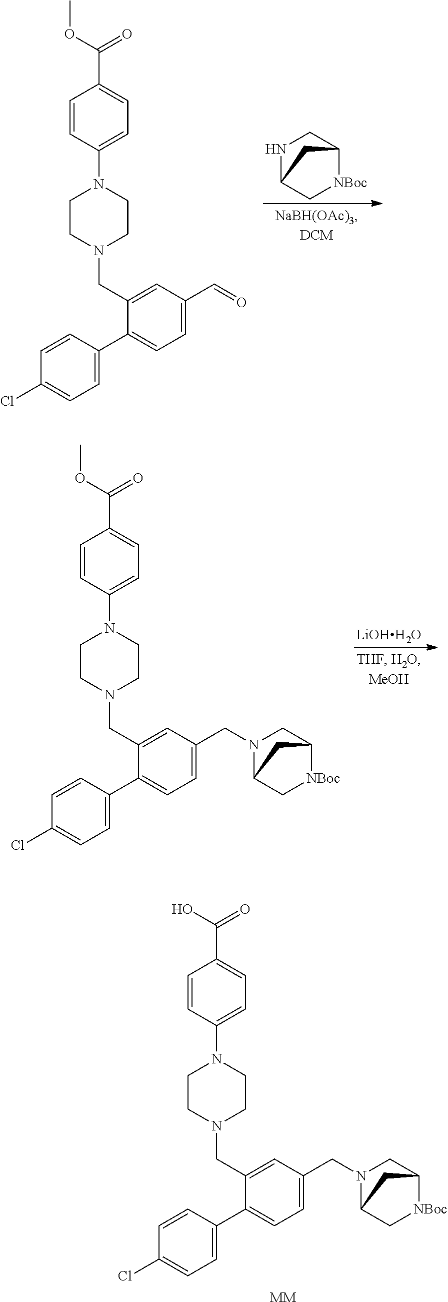

### Step 1—(1R,4R)-tert-butyl 5-((4′-chloro-2-((4-(4-(methoxycarbonyl)phenyl)piperazin-1-yl)methyl)-[1,1′-biphenyl]-4-yl)methyl)-2,5-diazabicyclo[2.2.1]heptane-2-carboxylate

To a solution of (1R,4R)-tert-butyl 2,5-diazabicyclo[2.2.1]heptane-2-carboxylate (159 mg, 802 μmol, CAS #134003-84-2) in DCM (5 mL) was added NaBH(OAc)₃ (283 mg, 1.34 mmol) and methyl 4-(4-((4′-chloro-4-formyl-[1,1′-biphenyl]-2-yl)methyl)piperazin-1-yl)benzoate (300 mg, 535 μmol, synthesized via Steps 1-2 of Intermediate LK). The reaction was then stirred at 25° C. for 12 hrs. On completion, the reaction mixture was quenched with MeOH (2 mL), and then concentrated under reduced pressure to give a residue. The residue was purified by column chromatography (SiO₂, Petroleum ether/Ethyl acetate=2/1 to 0/1) to give the title compound (230 mg, 67% yield) as a colorless solid. LC-MS (ESI⁺) m/z 631.5. (M+H)⁺.

### Step 2—4-(4-((4-(((1R,4R)-5-(tert-butoxycarbonyl)-2,5-diazabicyclo[2.2.1]heptan-2-yl)methyl)-4′-chloro-[1,1′-biphenyl]-2-yl)methyl)piperazin-1-yl)benzoic Acid

To a solution of (1R,4R)-tert-butyl 5-((4′-chloro-2-((4-(4-(methoxycarbonyl)phenyl)piperazin-1-yl)methyl)-[1,1′-biphenyl]-4-yl)methyl)-2,5-diazabicyclo[2.2.1]heptane-2-carboxylate (230 mg, 364 μmol) in THF (1 mL), H₂O (0.4 mL) and MeOH (1 mL) was added LiOH·H₂O (76.5 mg, 1.82 mmol). The mixture was then stirred at 50° C. for 12 hrs. On completion, the reaction mixture was added HCl (1N) until the pH was 6.0, then diluted by water (20 mL) and extracted by dichloromethane (3×20 mL). The combined organic layers were dried over anhydrous sodium sulfate, filtered and the filtrate was concentrated in vacuo to give the title compound (200 mg) as a white solid. LC-MS (ESI⁺) m/z 617.5. (M+H)⁺.

### Synthesis of 4-(4-((4-((1R,4R)-2,5-diazabicyclo[2.2.1]heptan-2-ylmethyl)-4′-chloro-[1,1′-biphenyl]-2-yl)methyl)piperazin-1-yl)-N-(4-(((R)-4-(dimethylamino)-1-(phenylthio)butan-2-yl)amino)-3-nitrophenyl)sulfonyl)benzamide (Intermediate MN)

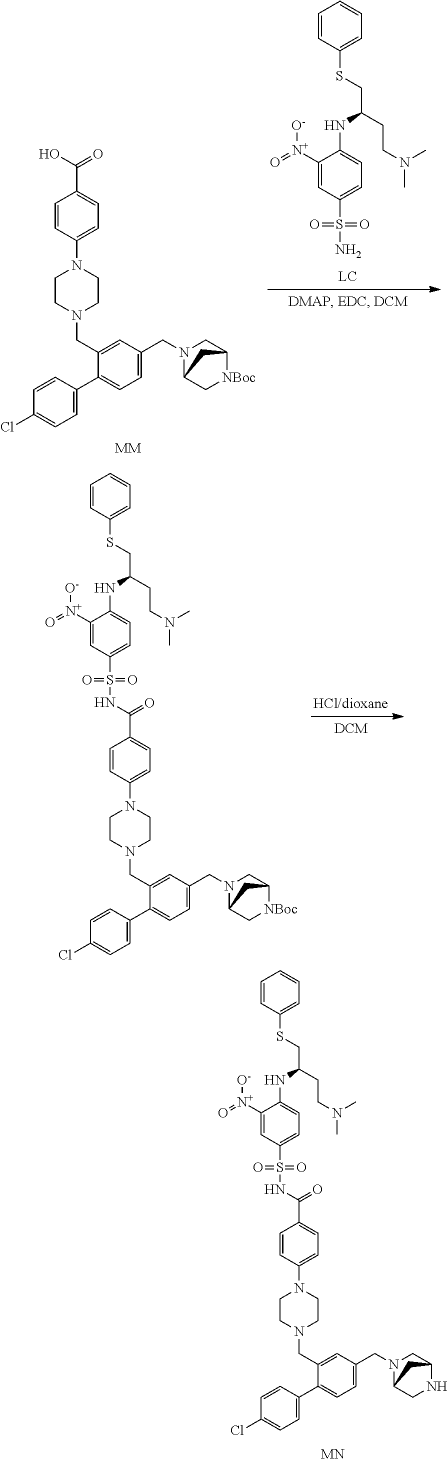

### Step 1—(1R,4R)-tert-butyl 5-((4′-chloro-2-((4-(4-(((4-(((R)-4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)methyl)-2,5-diazabicyclo[2.2.1]heptane-2-carboxylate

To a solution of 4-(4-((4-(((1R,4R)-5-(tert-butoxycarbonyl)-2,5-diazabicyclo[2.2.1]heptan-2-yl)methyl)-4′-chloro-[1,1′-biphenyl]-2-yl)methyl)piperazin-1-yl)benzoic acid (160 mg, 259 μmol, Intermediate MM) in DCM (1 mL) was added DMAP (79.2 mg, 648 μmol), EDC (80.5 mg, 518 μmol, 92 uL) and (R)-4-((4-(dimethylamino)-1-(phenylthio)butan-2-yl)amino)-3-nitrobenzenesulfonamide (165 mg, 389 μmol, Intermediate LC). The mixture was stirred at 25° C. for 12 hrs. On completion, the reaction mixture was concentrated under reduced pressure to give a residue. The residue was purified by reversed-phase HPLC (0.8 g/L ammonium bicarbonate) to give the title compound (50 mg, 18% yield) as a yellow solid. LC-MS (ESI⁺) m/z 1023.7. (M+H)⁺.

### Step 2—4-(4-((4-((1R,4R)-2,5-diazabicyclo[2.2.1]heptan-2-ylmethyl)-4′-chloro-[1,1′-biphenyl]-2-yl)methyl)piperazin-1-yl)-N-((4-(((R)-4-(dimethylamino)-1-(phenylthio)butan-2-yl)amino)-3-nitrophenyl)sulfonyl)benzamide

To a solution of (1R,4R)-tert-butyl 5-((4′-chloro-2-((4-(4-(((4-(((R)-4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)methyl)-2,5-diazabicyclo[2.2.1]heptane-2-carboxylate (45 mg, 44.0 μmol) in DCM (1 mL) was added HCl/dioxane (4 M, 11.0 uL). The mixture was stirred at 25° C. for 1 hr. On completion, the reaction mixture was concentrated under reduced pressure to give the title compound (40 mg, HCl) as a yellow solid. LC-MS (ESI⁺) m/z 923.5. (M+H)⁺.

### Synthesis of 4-(4-((4-((9-(Tert-butoxycarbonyl)-3,9-diazaspiro[5.5]undecan-3-yl)methyl)-4′-chloro-[1,1′-biphenyl]-2-yl)methyl)piperazin-1-yl)benzoic Acid (Intermediate MO)

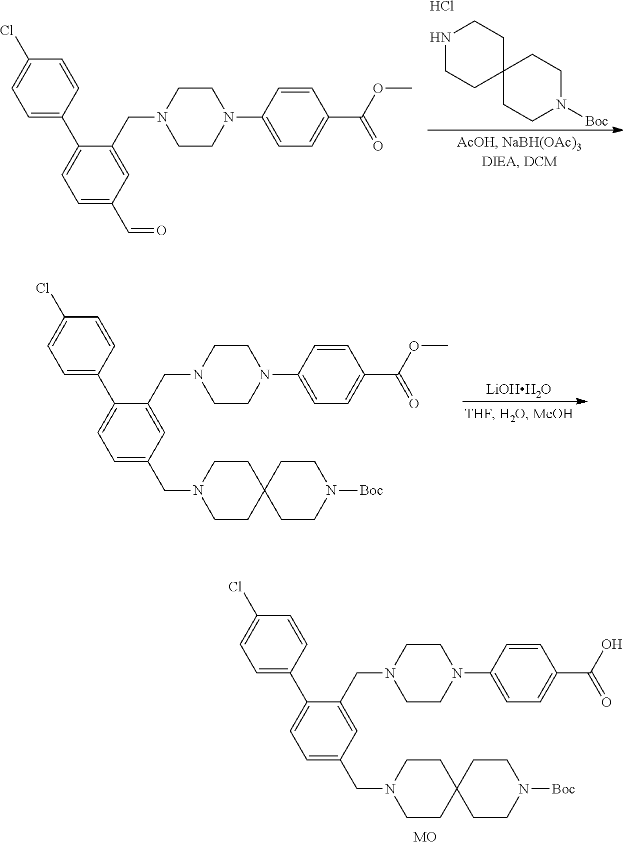

### Step 1—Tert-butyl 9-((4′-chloro-2-((4-(4-(methoxycarbonyl)phenyl)piperazin-1-yl)methyl)-[1,1′-biphenyl]-4-yl)methyl)-3,9-diazaspiro[5.5]undecane-3-carboxylate

A solution of methyl 4-(4-((4′-chloro-4-formyl-[1,1′-biphenyl]-2-yl)methyl)piperazin-1-yl)benzoate (100 mg, 223 μmol, synthesized via Steps 1-2 of Intermediate LK), tert-butyl 3,9-diazaspiro[5.5]undecane-3-carboxylate hydrochloride (77.7 mg, 267 μmol, HCl, CAS #173405-78-2), DIEA (86.4 mg, 668 μmol, 116 uL), AcOH (6.69 mg, 111 μmol, 6.37 uL), and NaBH(OAc)₃ (142 mg, 668 μmol) in DCM (1 mL) was stirred at 25° C. for 12 hrs. On completion, the reaction mixture was quenched with water (10 mL) and extracted by ethyl acetate (2×10 mL). The combined organic layers were washed with brine (10 mL) and dried over anhydrous sodium sulfate, filtered and the filtrate was concentrated in vacuo. The residue was purified by column chromatography (SiO₂, Dichloromethan:Methanol=50/1 to 10/1) to give the title compound (120 mg) as a white solid. LC-MS (ESI⁺) m/z 687.4 (M+H)⁺.

### Step 2—4-(4-((4-((9-(Tert-butoxycarbonyl)-3,9-diazaspiro[5.5]undecan-3-yl)methyl)-4′-chloro-[1,1′-biphenyl]-2-yl)methyl)piperazin-1-yl)benzoic Acid

A solution of tert-butyl 9-((4′-chloro-2-((4-(4-(methoxycarbonyl)phenyl)piperazin-1-yl)methyl)-[1,1′-biphenyl]-4-yl)methyl)-3,9-diazaspiro[5.5]undecane-3-carboxylate (100 mg, 145 μmol), LiOH·H₂O (30.5 mg, 727 μmol) in THF (0.5 mL), H₂O (0.5 mL), MeOH (0.5 mL) was stirred at 25° C. for 12 hrs. On completion, HCl (1N) was added to the reaction mixture until the pH=7, then the mixture was diluted with water (10 mL) and extracted with ethyl acetate/dichloromethane (2×10 mL). The combined organic layers were dried over anhydrous sodium sulfate, filtered and the filtrate was concentrated in vacuo to give the title compound (100 mg) as a light yellow solid. ¹H NMR (400 MHz, DMSO-d6) δ=11.03-10.78 (m, 1H), 8.34-8.22 (m, 1H), 7.84-7.75 (m, 3H), 7.55 (d, J=8.4 Hz, 2H), 7.46-7.36 (m, 3H), 6.94 (d, J=9.2 Hz, 2H), 4.37 (s, 2H), 3.85 (d, J=11.2 Hz, 2H), 3.30 (s, 4H), 3.20 (s, 2H), 2.93 (d, J=5.6 Hz, 2H), 1.83-1.73 (m, 4H), 1.56 (s, 2H), 1.38 (s, 9H), 1.27 (d, J=5.2 Hz, 2H).

### Synthesis of (R)-4-(4-((4-((3,9-diazaspiro[5.5]undecan-3-yl)methyl)-4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide (Intermediate MP)

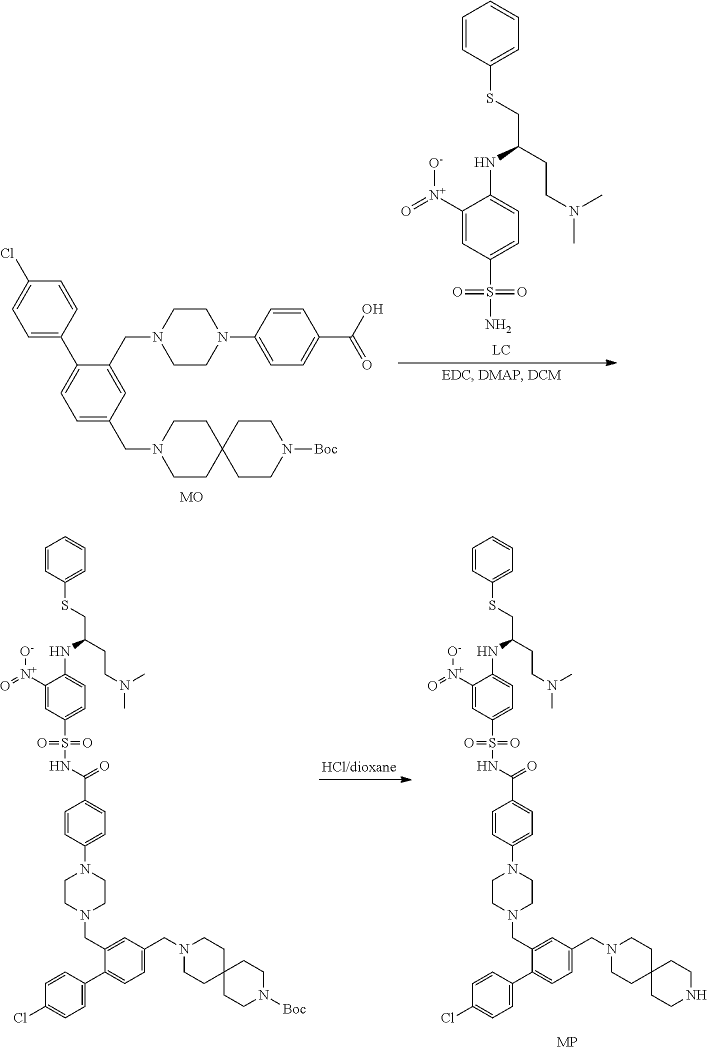 [SMILES: CC(C)(C)OC(=O)N1CCC2(CCN(Cc3ccc(-c4ccc(Cl)cc4)c(CN4CCN(c5ccc(C(=O)O)cc5)CC4)c3)CC2)CC1 ; CN(C)CC[C@H](CSc1ccccc1)Nc1ccc(S(N)(=O)=O)cc1[N+](=O)[O-] ; CN(C)CC[C@H](CSc1ccccc1)Nc1ccc(S(=O)(=O)NC(=O)c2ccc(N3CCN(Cc4cc(CN5CCC6(CC5)CCN(C(=O)OC(C)(C)C)CC6)ccc4-c4ccc(Cl)cc4)CC3)cc2)cc1[N+](=O)[O-] ; CN(C)CC[C@H](CSc1ccccc1)Nc1ccc(S(=O)(=O)NC(=O)c2ccc(N3CCN(Cc4cc(CN5CCC6(CCNCC6)CC5)ccc4-c4ccc(Cl)cc4)CC3)cc2)cc1[N+](=O)[O-]]

### Step 1—Tert-butyl (R)-9-((4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)methyl)-3,9-diazaspiro[5.5]undecane-3-carboxylate

A solution of (R)-4-((4-(dimethylamino)-1-(phenylthio)butan-2-yl)amino)-3-nitrobenzenesulfonamide (66.7 mg, 59.4 μmol, Intermediate LC), 4-(4-((4-((9-(tert-butoxycarbonyl)-3,9-diazaspiro[5.5]undecan-3-yl)methyl)-4′-chloro-[1,1′-biphenyl]-2-yl)methyl)piperazin-1-yl)benzoic acid (30.3 mg, 71.3 μmol, Intermediate MO), EDC (18.4 mg, 119 μmol, 21.0 uL), and DMAP (18.1 mg, 148 μmol) in DCM (0.5 mL) was stirred at 25° C. for 12 hrs. On completion, the reaction mixture was quenched with sat, NH₄Cl (10 mL) and extracted by dichloromethane (2×10 mL). The combined organic layers were washed by brine (20 mL) and dried over anhydrous sodium sulfate, filtered and the filtrate was concentrated in vacuo to give the crude residue. The residue was purified by column chromatography prep-TLC (SiO₂, Dichloromethan:Methanol=10/1) to give the title compound (60.0 mg) as a yellow solid. LC-MS (ESI⁺) m/z 540.3 (M+H)⁺.

### Step 2—(R)-4-(4-((4-((3,9-diazaspiro[5.5]undecan-3-yl)methyl)-4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide

A solution of tert-butyl (R)-9-((4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)methyl)-3,9-diazaspiro[5.5]undecane-3-carboxylate (33.1 mg, 30.6 μmol) in HCl/dioxane (0.2 mL) was stirred at 25° C. for 1 hr. On completion, the reaction mixture was concentrated in vacuo to give the title compound (30 mg). LC-MS (ESI⁺) m/z 911.4 (M+H)⁺.

### Synthesis of (R)-1-(4-(N-(4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-carbonyl)sulfamoyl)phenyl)piperidine-4-carboxylic Acid (Intermediate MQ)

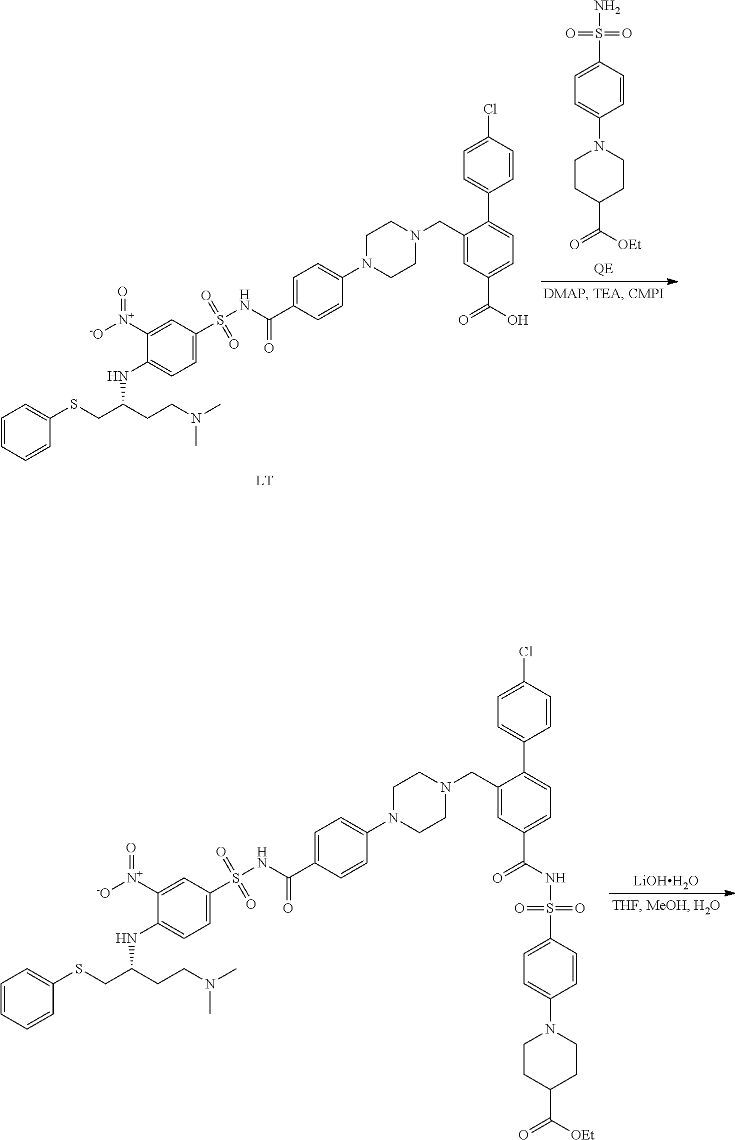

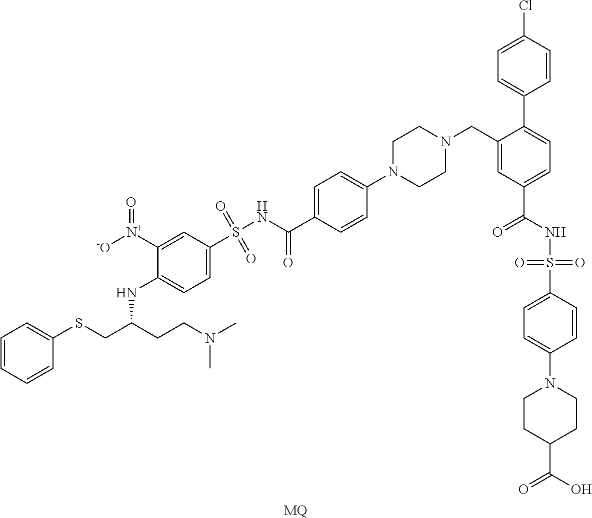

### Step 1—(R)-ethyl 1-(4-(N-(4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-carbonyl)sulfamoyl)phenyl)piperidine-4-carboxylate

To a solution of (R)-4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-carboxylic acid (120 mg, 140 μmol, Intermediate LT) in DCM (3 mL) was added 2-chloro-1-methylpyridinium iodide (53.6 mg, 210 μmol, CAS #14338-32-0), DMAP (17.1 mg, 140 μmol), TEA (42.5 mg, 420 μmol) and ethyl 1-(4-sulfamoylphenyl)piperidine-4-carboxylate (65.4 mg, 210 μmol, Intermediate QE). The mixture was then stirred at 25° C. for 12 hrs. On completion, the reaction mixture was concentrated in vacuo to remove the solvent. Then the crude residue was purified by reversed-phase HPLC (0.1% FA condition) to give the title compound (142 mg, 85% yield) as a yellow solid. LC-MS (ESI⁺) m/z 576.7 (M/2+H)⁺.

### Step 2—(R)-1-(4-(N-(4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-carbonyl)sulfamoyl)phenyl)piperidine-4-carboxylic Acid

To a solution of (R)-ethyl 1-(4-(N-(4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-carbonyl)sulfamoyl)phenyl)piperidine-4-carboxylate (140 mg, 122 μmol) in THF (4 mL), MeOH (4 mL) and H₂O (1.6 mL) was added LiOH·H₂O (40.8 mg, 972 μmol). The mixture was then stirred at 50° C. for 2 hrs. On completion, the reaction mixture was concentrated in vacuo to remove the solvent. Then the reaction mixture was diluted with water (30 mL) and extracted by dichloromethane (3×30 mL). The combined organic layers were washed with brine (60 mL), dried over anhydrous sodium sulfate, filtered and the filtrate was concentrated in vacuo to give the title compound (120 mg) as a white solid. LC-MS (ESI⁺) m/z 563.4 (M/2+H)⁺.

### Synthesis of Tert-butyl 4-(3-bromopropyl)piperazine-1-carboxylate (Intermediate MR)

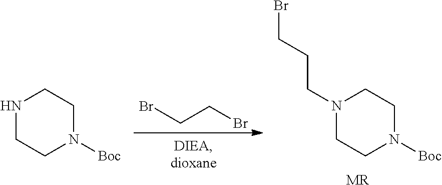

To a solution of tert-butyl piperazine-1-carboxylate (2 g, 10.7 mmol, CAS #57260-71-6) and 1,2-dibromoethane (6.05 g, 32.2 mmol, CAS #106-93-4) in dioxane (10 mL) was added DIEA (2.78 g, 21.5 mmol). The mixture was then stirred at 90° C. for 12 hrs. On completion, the mixture was concentrated to give a residue and purified by column chromatography (SiO₂, Petroleum ether/Ethyl acetate=1/0 to 1:1) to give the title compound (1.7 g, 52% yield) as a yellow solid. LC-MS (ESI⁺) m/z 307.0 (M+H)⁺.

### Synthesis of Tert-butyl (R)-(3-amino-4-(phenylthio)butyl)(methyl)carbamate (Intermediate MS)

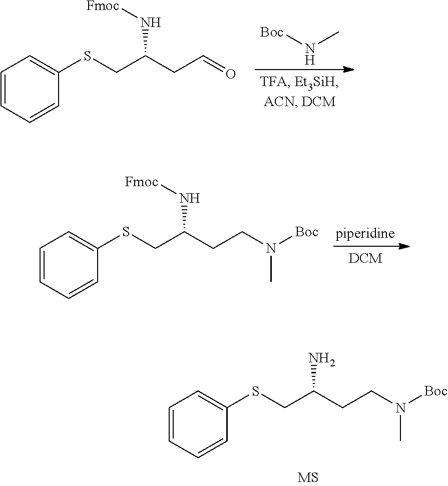

### Step 1—Tert-butyl (R)-(3-((((9H-fluoren-9-yl)methoxy) carbonyl)amino)-4-(phenylthio)butyl)(methyl)carbamate

To a solution of (9H-fluoren-9-yl)methyl (R)-(4-oxo-1-(phenylthio)butan-2-yl)carbamate (6.0 g, 14.4 mmol, synthesized via Steps 1-3 of Intermediate KK) in ACN (60 mL) and DCM (20 mL) was added tert-butyl methylcarbamate (3.77 g, 28.7 mmol, CAS #16066-84-5), Et₃SiH (8.35 g, 71.9 mmol) and TFA (4.92 g, 43.1 mmol) at 0° C. Then the mixture was stirred at 25° C. for 12 hrs. On completion, the mixture was concentrated to give a residue and purified by column chromatography (SiO₂, Petroleum ether/Ethyl acetate=1/0 to 1:1) to give the title compound (5.5 g, 68% yield) as a white solid. LC-MS (ESI⁺) m/z 433.4 (M-99)⁺.

### Step 2—Tert-butyl (R)-(3-amino-4-(phenylthio)butyl)(methyl)carbamate

To a solution of tert-butyl (R)-(3-((((9H-fluoren-9-yl)methoxy) carbonyl)amino)-4-(phenylthio)butyl)(methyl)carbamate (5.5 g, 10.3 mmol) in DCM (50 mL) was added piperidine (4.40 g, 51.6 mmol, 5.10 mL). The mixture was then stirred at 25° C. for 12 hrs. On completion, the mixture was concentrated to give a residue and purified by reversed-phase HPLC (0.8 g/L ammonium bicarbonate) to give the title compound (2.6 g, 76% yield) as a yellow oil. LC-MS (ESI⁺) m/z 310.9 (M+H)⁺.

### Synthesis of Tert-butyl (R)-methyl (4-(phenylthio)-3-((4-sulfamoyl-2-((trifluoromethyl)sulfonyl)phenyl)amino)butyl)carbamate (Intermediate MT)

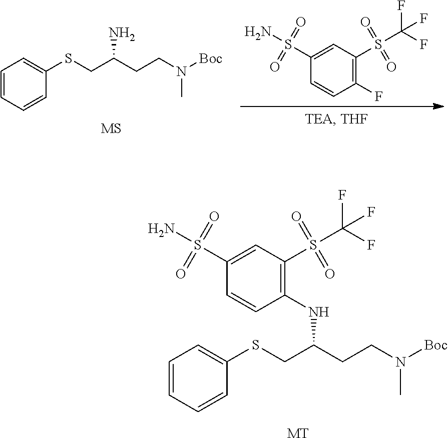

To a solution of tert-butyl (R)-(3-amino-4-(phenylthio)butyl)(methyl)carbamate (1.4 g, 4.51 mmol, Intermediate MS) in ACN (20 mL) was added DIEA (2.91 g, 22.6 mmol) and 4-fluoro-3-((trifluoromethyl)sulfonyl)benzenesulfonamide (2.08 g, 6.76 mmol, CAS #1027345-08-9). The mixture was stirred at 80° C. for 6 hrs. On completion, the mixture was concentrated to give a residue and purified by column chromatography (SiO₂, Petroleum ether/Ethyl acetate=10/1 to 1:1) to give the title compound (1.2 g, 31% yield) as a white solid. LC-MS (ESI⁺) m/z 620.1 (M+23)⁺.

### Synthesis of 4-(4-((R)-(4′-chloro-[1,1′-biphenyl]-2-yl)(hydroxy)methyl)piperidin-1-yl)-N-((4-(((R)-4-(methylamino)-1-(phenylthio)butan-2-yl)amino)-3-((trifluoromethyl)sulfonyl)phenyl)sulfonyl)benzamide (Intermediate MU)

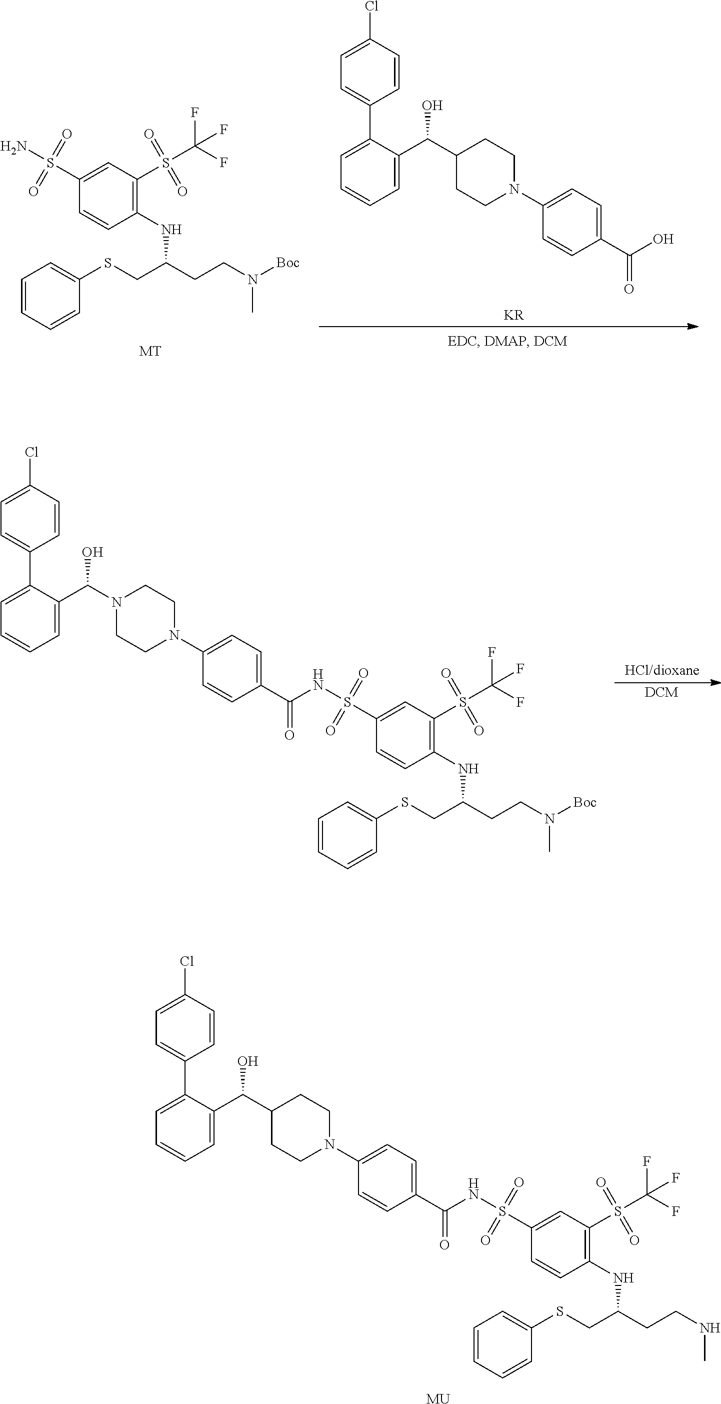

### Step 1—Tert-butyl ((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)carbamate

To a solution of (R)-4-(4-((4′-chloro-[1,1′-biphenyl]-2-yl)(hydroxy)methyl)piperidin-1-yl)benzoic acid (409 mg, 970 μmol, Intermediate KR) in DCM (8 mL) was added DMAP (296 mg, 2.43 mmol), EDC (301 mg, 1.94 mmol) and tert-butyl (R)-methyl (4-(phenylthio)-3-((4-sulfamoyl-2-((trifluoromethyl)sulfonyl)phenyl)amino)butyl)carbamate (580 mg, 970 μmol, Intermediate MT). The mixture was then stirred at 40° C. for 2 hrs. On completion, the mixture was concentrated to give a residue and purified by reversed-phase HPLC (0.1% FA condition) to give the title compound (700 mg, 69% yield) as a white solid. LC-MS (ESI⁺) m/z 1001.3 (M+H)⁺.

### Step 2—4-(4-((R)-(4′-chloro-[1,1′-biphenyl]-2-yl)(hydroxy)methyl)piperidin-1-yl)-N-((4-(((R)-4-(methylamino)-1-(phenylthio)butan-2-yl)amino)-3-((trifluoromethyl)sulfonyl)phenyl)sulfonyl)benzamide

To a solution of tert-butyl ((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)carbamate (700 mg, 699 μmol) in DCM (10 mL) was added HCl/Dioxane (8 M, 1 mL). The mixture was then stirred at 25° C. for 10 min. On completion, the mixture was concentrated to remove the organic solvent and give the title compound (750 mg, HCl) as a yellow solid. LC-MS (ESI⁺) m/z 901.1 (M+H)⁺.

### Synthesis of 4-(4-((R)-(4′-chloro-[1,1′-biphenyl]-2-yl)(hydroxy)methyl)piperidin-1-yl)-N-((4-(((R)-4-(methyl (3-(piperazin-1-yl)propyl)amino)-1-(phenylthio)butan-2-yl)amino)-3-((trifluoromethyl)sulfonyl)phenyl)sulfonyl)benzamide (Intermediate MV)

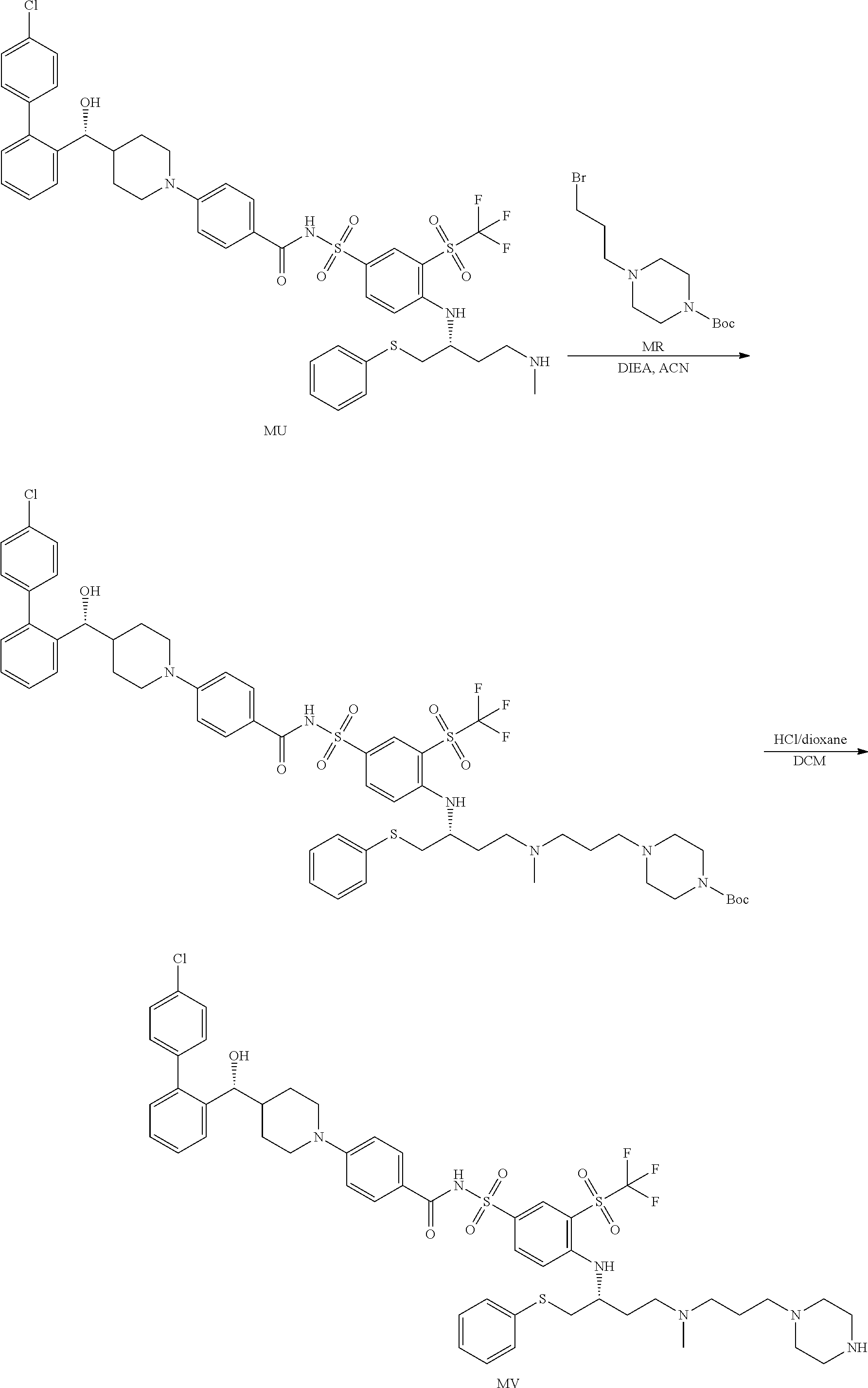

### Step 1—Tert-butyl 4-(3-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)propyl)piperazine-1-carboxylate

A mixture of 4-(4-((R)-(4′-chloro-[1,1′-biphenyl]-2-yl)(hydroxy)methyl)piperidin-1-yl)-N-((4-(((R)-4-(methylamino)-1-(phenylthio)butan-2-yl)amino)-3-((trifluoromethyl)sulfonyl)phenyl)sulfonyl)benzamide (700 mg, 777 μmol, Intermediate MU), tert-butyl 4-(3-bromopropyl)piperazine-1-carboxylate (477 mg, 1.55 mmol, Intermediate MR), KI (19.3 mg, 116 μmol), and DIEA (502 mg, 3.88 mmol) in ACN (10 mL) was stirred at 50° C. for 12 hrs. On completion, the mixture was concentrated to give a residue and purified by column chromatography (SiO₂, Dichloromethane: Methanol=10/1) to give the title compound (700 mg, 79% yield) as a yellow solid. LC-MS (ESI⁺) m/z 1127.6 (M+23)⁺.

### Step 2—4-(4-((R)-(4′-chloro-[1,1′-biphenyl]-2-yl)(hydroxy)methyl)piperidin-1-yl)-N-((4-(((R)-4-(methyl (3-(piperazin-1-yl)propyl)amino)-1-(phenylthio)butan-2-yl)amino)-3-((trifluoromethyl)sulfonyl)phenyl)sulfonyl)benzamide

To a solution of tert-butyl 4-(3-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)propyl)piperazine-1-carboxylate (200 mg, 177 μmol) in DCM (2 mL) was added HCl/dioxane (4 M, 200 uL) at 25° C. then the stirred for 2 hrs. On completion, the mixture was concentrated to give the title compound (200 mg, HCl) as a white solid. LC-MS (ESI⁺) m/z 514.9 (M/2+H)⁺.

### Synthesis of 4-(4-((S)-(4′-chloro-[1,1′-biphenyl]-2-yl)(hydroxy)methyl)piperidin-1-yl)-N-((4-(((R)-4-(methyl (3-(piperazin-1-yl)propyl)amino)-1-(phenylthio)butan-2-yl)amino)-3-(trifluoromethyl)sulfonyl)phenyl)sulfonyl)benzamide (Intermediate MW)

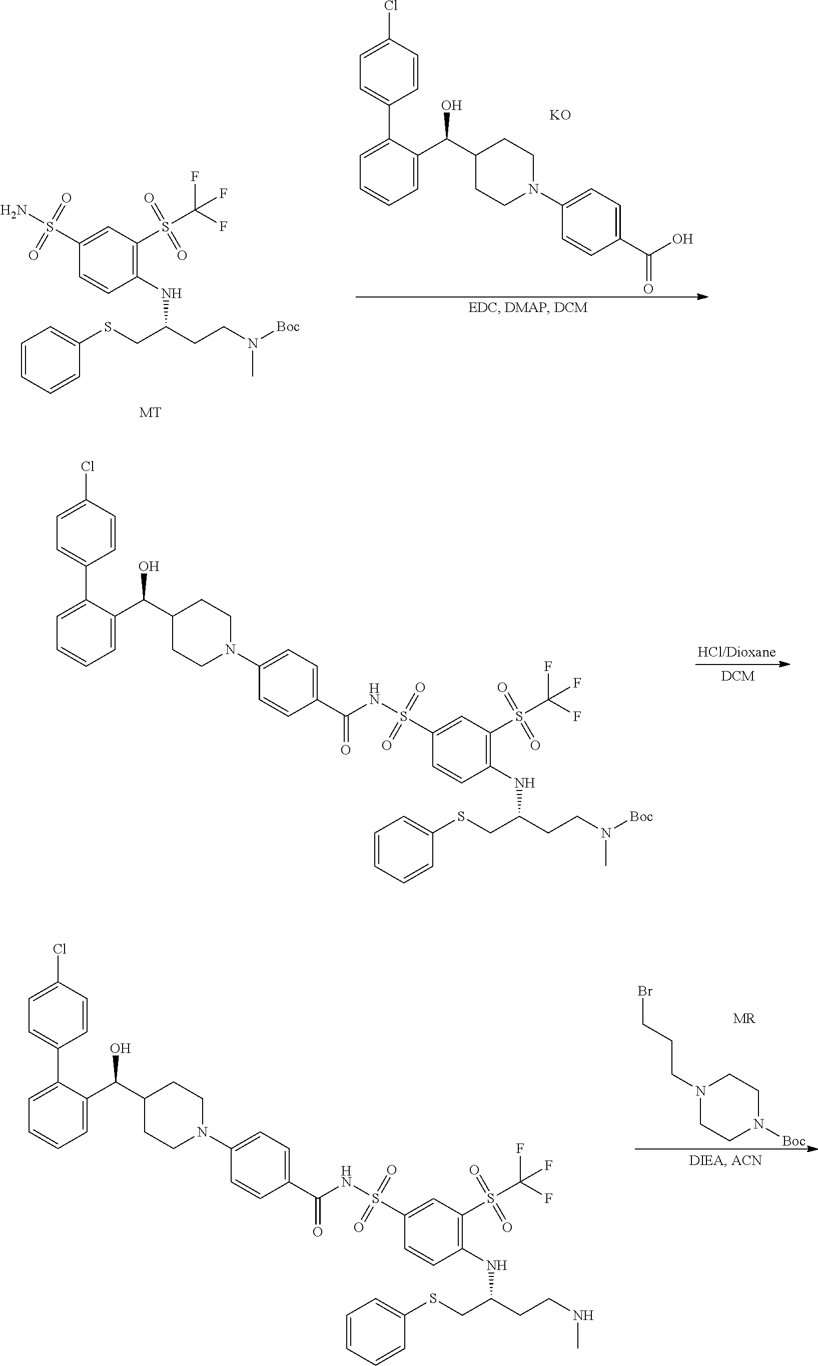

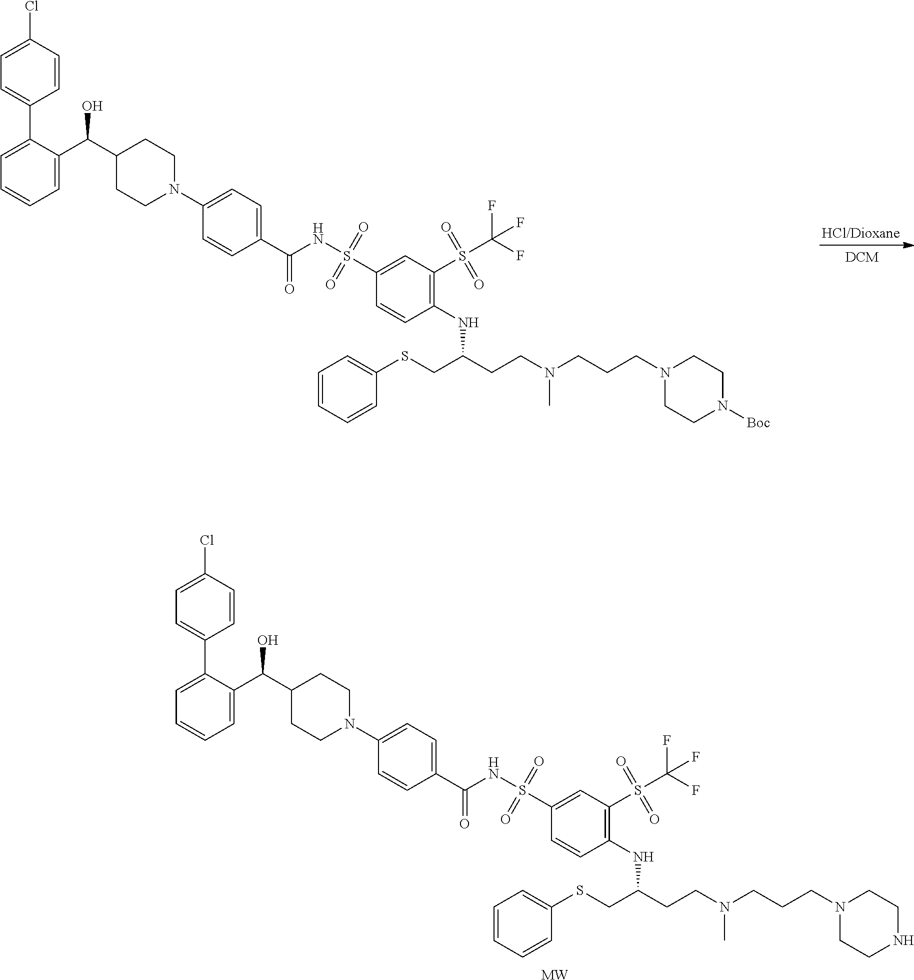

### Step 1—Tert-butyl ((R)-3-((4-(N-(4-(4-((S)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)carbamate

To a solution of (S)-4-(4-((4′-chloro-[1,1′-biphenyl]-2-yl)(hydroxy)methyl)piperidin-1-yl)benzoic acid (353 mg, 836 μmol, Intermediate KO) in DCM (4 mL) was added DMAP (255 mg, 2.09 mmol), EDC (260 mg, 1.67 mmol) and tert-butyl (R)-methyl (4-(phenylthio)-3-((4-sulfamoyl-2-((trifluoromethyl)sulfonyl)phenyl)amino)butyl)carbamate (500 mg, 836 μmol, Intermediate MT). The mixture was stirred at 40° C. for 2 hrs. On completion, the mixture was concentrated to give a residue and purified by reversed-phase HPLC (0.1% FA condition) to give the title compound (600 mg, 64% yield) as a white solid. LC-MS (ESI⁺) m/z 1001.2 (M+H)⁺.

### Step 2—4-(4-((S)-(4′-chloro-[1,1′-biphenyl]-2-yl)(hydroxy)methyl)piperidin-1-yl)-N-((4-(((R)-4-(methylamino)-1-(phenylthio)butan-2-yl)amino)-3-((trifluoromethyl)sulfonyl)phenyl)sulfonyl)benzamide

To a solution of tert-butyl ((R)-3-((4-(N-(4-(4-((S)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)carbamate (600 mg, 599 μmol) in DCM (10 mL) was added HCl/Dioxane (4 M, 2 mL). The mixture was stirred at 25° C. for 12 hrs. On completion, the mixture was concentrated to give the title compound (700 mg, HCl) as a white solid. LC-MS (ESI⁺) m/z 901.1 (M+H)⁺.

### Step 3—Tert-butyl 4-(3-(((R)-3-((4-(N-(4-(4-((S)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)propyl)piperazine-1-carboxylate

A mixture of 4-(4-((R)-(4′-chloro-[1,1′-biphenyl]-2-yl)(hydroxy)methyl)piperidin-1-yl)-N-((4-(((R)-4-(methylamino)-1-(phenylthio)butan-2-yl)amino)-3-((trifluoromethyl)sulfonyl)phenyl)sulfonyl)benzamide (700 mg, 776 μmol), tert-butyl 4-(3-bromopropyl)piperazine-1-carboxylate (477 mg, 1.55 mmol, Intermediate MR), KI (19.3 mg, 116 μmol), and DIEA (502 mg, 3.88 mmol) in ACN (10 mL) was stirred at 50° C. for 12 hrs. On completion, the mixture was concentrated to give a residue and purified by column chromatography (SiO₂, Dichloromethan:Methanol=10/1) to give the title compound (500 mg, 57% yield) as a yellow solid. LC-MS (ESI⁺) m/z 1127.6 (M+23)⁺.

### Step 4—4-(4-((S)-(4′-chloro-[1,1′-biphenyl]-2-yl)(hydroxy)methyl)piperidin-1-yl)-N-((4-(((R)-4-(methyl (3-(piperazin-1-yl)propyl)amino)-1-(phenylthio)butan-2-yl)amino)-3-((trifluoromethyl)sulfonyl)phenyl)sulfonyl)benzamide

To a solution of tert-butyl 4-(3-(((R)-3-((4-(N-(4-(4-((S)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)propyl)piperazine-1-carboxylate (200 mg, 177 μmol) in DCM (2 mL) was added HCl/dioxane (4 M, 200 uL) and the mixture was stirred at 25° C. for 2 hrs. On completion, the mixture was concentrated to give the title compound (200 mg, HCl) as a white solid. LC-MS (ESI⁺) m/z 514.5 (M/2+H)⁺.

### Synthesis of (R)-4-(4-((4-((4-(tert-butoxycarbonyl)piperazin-1-yl)methyl)-4′-chloro-[1,1′-biphenyl]-2-yl)(hydroxy)methyl)piperidin-1-yl)benzoic Acid (Intermediate MX)

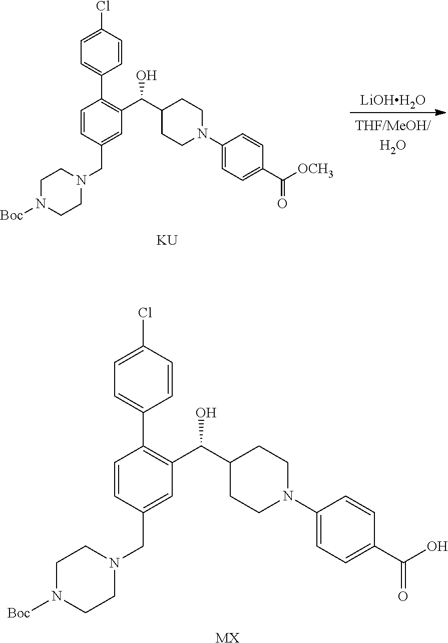

To a solution of (R)-tert-butyl 4-((4′-chloro-2-(hydroxy(1-(4-(methoxycarbonyl)phenyl)piperidin-4-yl)methyl)-[1,1′-biphenyl]-4-yl)methyl)piperazine-1-carboxylate (300 mg, 473 μmol, Intermediate KU) in THF (2 mL), H₂O (0.5 mL) and MeOH (0.5 mL) was added LiOH·H₂O (79.3 mg, 1.89 mmol). The mixture was then stirred at 50° C. for 12 hrs. On completion, the reaction mixture was concentrated under reduced pressure to give a residue. The residue was purified by prep-HPLC (neutral condition) to give the title compound (290 mg, 98% yield) as a white solid. LC-MS (ESI⁺) m/z 620.5 (M+H)⁺.

### Synthesis of 4-(4-((R)-(4′-chloro-4-(piperazin-1-ylmethyl)-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide (Intermediate MY)

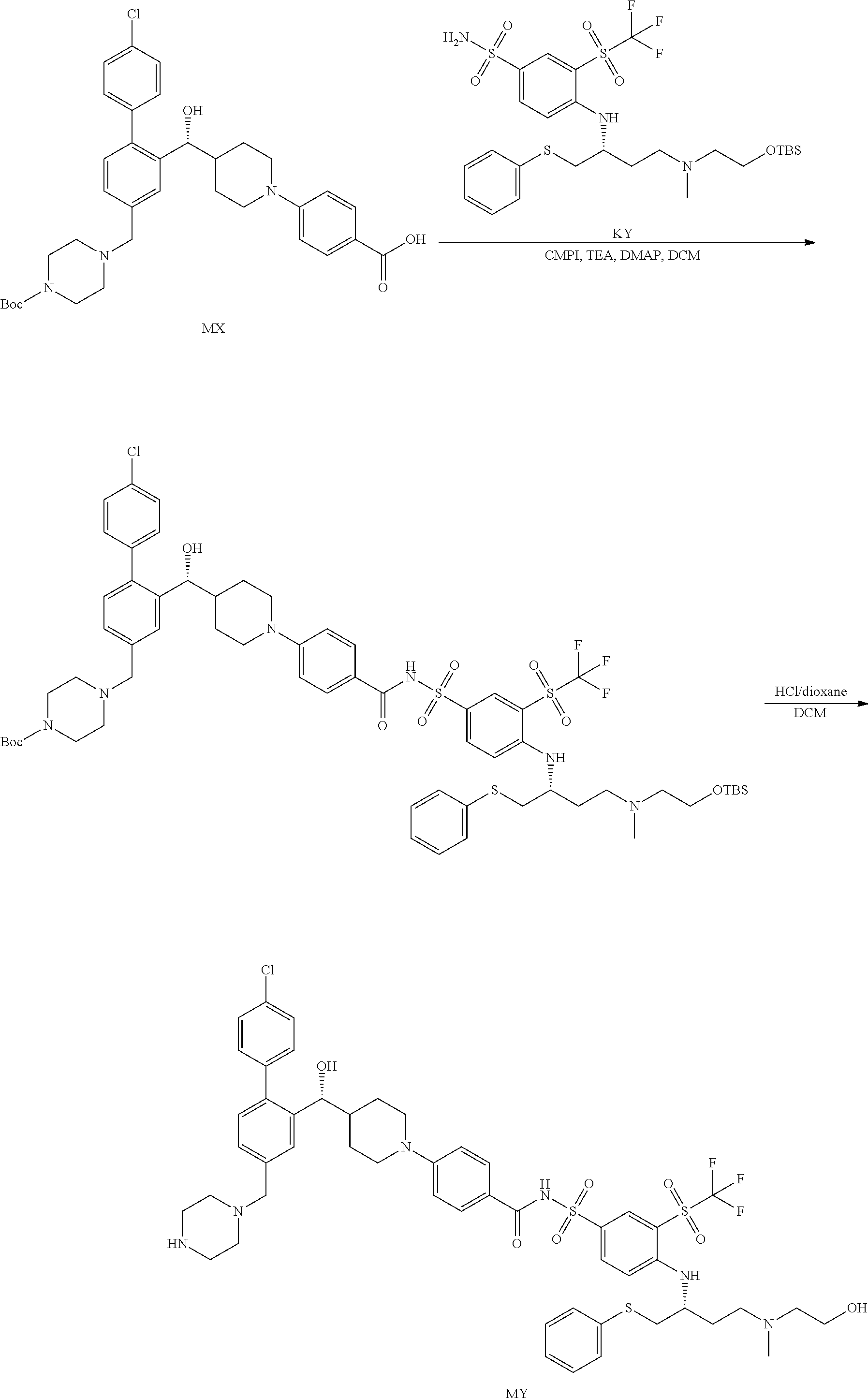

### Step 1—Tert-butyl 4-((2-((R)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)methyl)piperazine-1-carboxylate

To a solution of (R)-4-(4-((4-((4-(tert-butoxycarbonyl)piperazin-1-yl)methyl)-4′-chloro-[1,1′-biphenyl]-2-yl)(hydroxy)methyl)piperidin-1-yl)benzoic acid (100 mg, 161 μmol, Intermediate MX) in DCM (2 mL) was added 2-chloro-1-methyl-pyridin-1-ium;iodide (61.7 mg, 241 μmol). TEA (48 mg, 483 μmol, 67.3 uL), DMAP (19.7 mg, 161 μmol) and (R)-4-((4-((2-((tert-butyldimethylsilyl)oxy)ethyl)(methyl)amino)-1-(phenylthio)butan-2-yl)amino)-3-((trifluoromethyl)sulfonyl)benzenesulfonamide (105 mg, 161 μmol, Intermediate KY). The mixture was then stirred at 25° C. for 2 hrs. On completion, the reaction mixture was concentrated under reduced pressure to give a residue. The residue was purified by prep-HPLC (0.1% FA condition) to give the title compound (200 mg, 78% yield) as a white solid. LC-MS (ESI⁺) m/z 1259.5 (M+H)⁺.

### Step 2—4-(4-((R)-(4′-chloro-4-(piperazin-1-ylmethyl)-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide

To a solution of tert-butyl 4-((2-((R)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)methyl)piperazine-1-carboxylate (80 mg, 63 μmol) in DCM (1 mL) was added HCl/dioxane (4 M, 15.9 uL). The mixture was then stirred at 25° C. for 2 hrs. On completion, the reaction mixture was concentrated under reduced pressure to give a residue. The residue was purified by prep-HPLC (column: Welch Xtimate C18 150×25 mm×5 um; mobile phase: [water (ammonia hydroxide v/v)-ACN]; B %: 20%-50%, 2 min) to give the title compound (50 mg, 74% yield) as a white solid. ¹H NMR (400 MHz, DMSO-d₆) δ=8.03 (d, J=2.0 Hz, 1H), 7.89 (dd, J=2.0, 9.2 Hz, 1H), 7.67 (d, J=8.8 Hz, 2H), 7.52 (s, 1H), 7.49 (d, J=8.4 Hz, 2H), 7.37-7.16 (m, 8H), 7.11 (d, J=7.6 Hz, 1H), 6.85-6.77 (m, 2H), 6.71 (d, J=9.2 Hz, 2H), 5.23 (d, J=4.4 Hz, 1H), 4.46-4.31 (m, 2H), 3.99 (d, J=5.6 Hz, 1H), 3.71 (d, J=12.0 Hz, 1H), 3.59 (s, 2H), 3.54 (s, 1H), 3.43-3.40 (m, 2H), 3.27 (d, J=5.6 Hz, 2H), 3.23-3.18 (m, 2H), 3.06 (s, 4H), 2.58-2.52 (m, 4H), 2.46-2.32 (m, 6H), 2.14 (s, 3H), 1.94-1.82 (m, 2H), 1.74-1.65 (m, 1H), 1.62-1.53 (m, 1H), 1.19-1.07 (m, 1H), 1.05-0.97 (m, 1H), 0.96-0.81 (m, 1H). LC-MS (ESI⁺) m/z 1044.2 (M+H)⁺.

### Synthesis of 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoic Acid (Intermediate MZ)

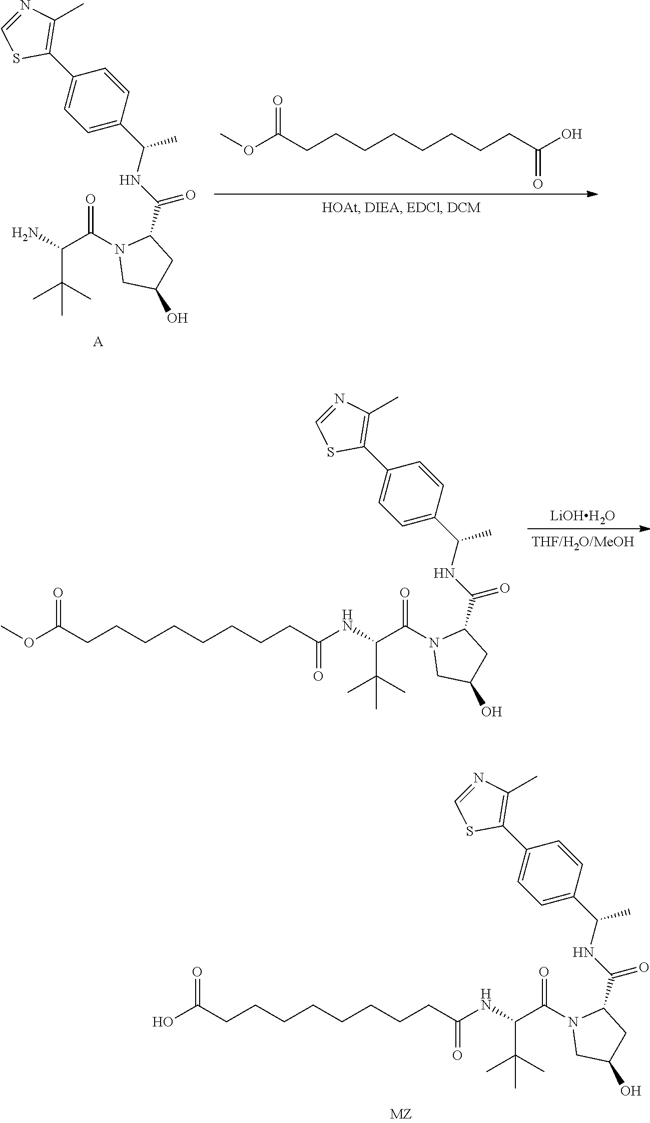

### Step 1—Methyl 10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoate

To a solution of 10-methoxy-10-oxo-decanoic acid (1.02 g, 4.72 mmol, CAS #818-88-2) in DCM (12 mL) was added HOAt (1.61 g, 11.8 mmol), DIEA (6.10 g, 47.2 mmol) and EDCI (2.26 g, 11.8 mmol). Then (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (2.1 g, 4.72 mmol, Intermediate A) was added and the mixture was stirred at 25° C. for 2 hrs. On completion, the reaction mixture was concentrated in vacuo to give the product. The crude product was purified by reversed-phase HPLC (0.1% FA condition) to the title compound (1.6 g, 46% yield, FA) as an off-white solid. LC-MS (ESI⁺) m/z 643.5 (M+H)⁺.

### Step 2—10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoic Acid

To a solution of methyl 10-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-10-oxo-decanoate (1.6 g, 2.49 mmol) in MeOH (3 mL), THF (12 mL) and H₂O (3 mL) was added LiOH H₂O (522 mg, 12.44 mmol). The mixture was then stirred at 25° C. for 2 hrs. On completion, the reaction mixture was quenched with 1M HCl at 25° C. until the pH=5, and then diluted with H₂O (10 mL) and extracted with dichloromethane (60 mL). The combined organic layers were dried over anhydrous sodium sulfate, filtered and the filtrate was concentrated under reduced pressure to give the title compound (1.6 g) as a white solid. LC-MS (ESI⁺) m/z 629.4 (M+H)⁺.

### Synthesis of Methyl (R)-4-(4-((4-(((tert-butoxycarbonyl)amino)methyl)-4′-chloro-[1,1′-biphenyl]-2-yl)(hydroxy)piperidin-1-yl)benzonate (Intermediate NA) and methyl (S)-4-(4-((4-(((tert-butoxycarbonyl)amino)methyl)-4′-chloro-[1,1′-biphenyl]-2-yl)(hydroxy)methyl)piperidin-1-yl)benzoate (Intermediate NB)

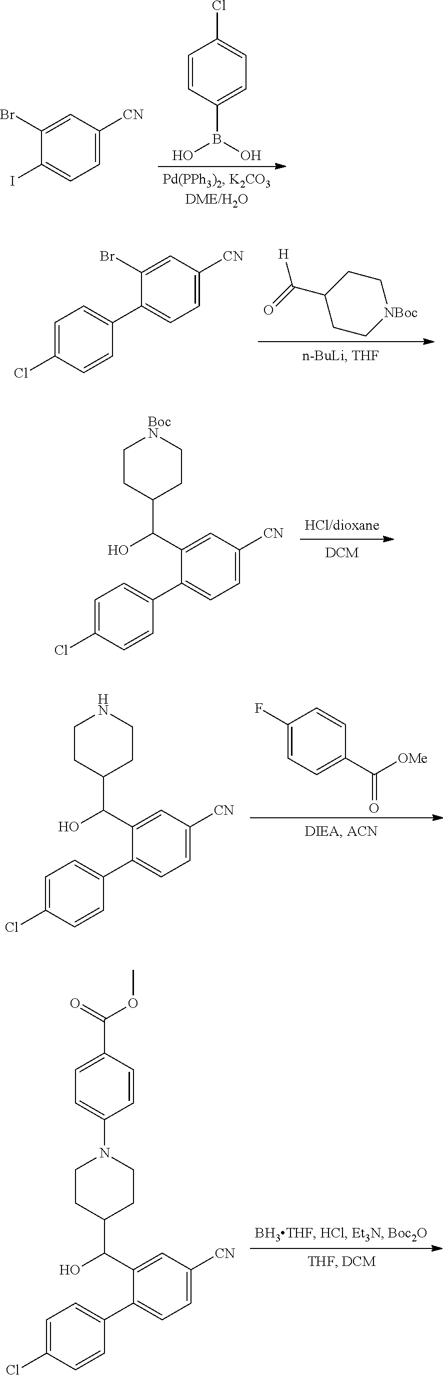

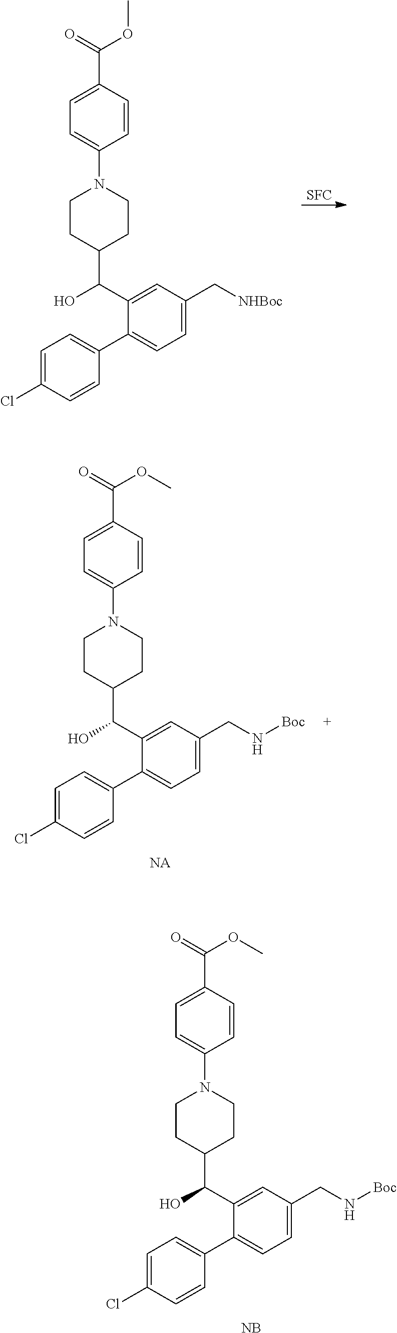 [SMILES: COC(=O)c1ccc(N2CCC(C(O)c3cc(CNC(=O)OC(C)(C)C)ccc3-c3ccc(Cl)cc3)CC2)cc1 ; "SFC" ; COC(=O)c1ccc(N2CCC([C@@H](O)c3cc(CNC(=O)OC(C)(C)C)ccc3-c3ccc(Cl)cc3)CC2)cc1 ; "NA" ; COC(=O)c1ccc(N2CCC([C@H](O)c3cc(CNC(=O)OC(C)(C)C)ccc3-c3ccc(Cl)cc3)CC2)cc1 ; "NB"]

### Step 1—2-Bromo-4′-chloro-[1,1′-biphenyl]-4-carbonitrile

To a solution of 3-bromo-4-iodobenzonitrile (10 g, 32.4 mmol, CAS #1000577-94-5) in H₂O (30 mL) and DME (150 mL) was added K₂CO₃ (11.2 g, 81.1 mmol), (4-chlorophenyl)boronic acid (6.09 g. 38.9 mmol, CAS #1679-18-1) and Pd(PPh₃)₂Cl₂ (1.14 g, 1.62 mmol). The mixture was then stirred at 80° C. for 3 hrs under N₂ atmosphere. On completion, the reaction mixture was quenched with H₂O (100 mL) at 25° C., and then extracted with DCM (3×200 mL). The combined organic layers were washed with brine (2×200 mL), dried over anhydrous sodium sulfate, filtered and the filtrate was concentrated under reduced pressure to give a residue. The residue was purified by column chromatography (SiO₂, Petroleum ether/DCM=1:0 to 10:1) to give the title compound (9 g, 81% yield) as a white solid. ¹H NMR (400 MHz, DMSO-d₆) δ=8.30 (d, J=1.6 Hz, 1H), 7.92 (dd, J=1.6, 8.0 Hz, 1H), 7.58-7.52 (m, 3H), 7.46-7.40 (m, 2H).

### Step 2—Tert-butyl 4-((4′-chloro-4-cyano-[1,1′-biphenyl]-2-yl)(hydroxy)methyl)piperidine-1-carboxylate

To a solution of 2-bromo-4′-chloro-[1,1′-biphenyl]-4-carbonitrile (12 g, 41.0 mmol) in THF (160 mL) was added n-BuLi (2.5 M, 24.6 mL) in −78° C. The mixture was then stirred at −78° C. in a 500 cc. three-necked round-bottomed flask under N₂ atmosphere for 1 hr. Then tert-butyl 4-formylpiperidine-1-carboxylate (8.75 g, 41.0 mmol, CAS #137076-22-3) was added at −78° C. The mixture was then stirred at −78° C. under N₂ atmosphere for 1 hr. On completion, the reaction mixture was quenched by NH₄Cl (50 mL) and extracted by ethyl acetate (3×150 mL). The combined organic layers were washed by brine (200 mL) and dried over anhydrous sodium sulfate, filtered and the filtrate was concentrated in vacuo to give the crude residue. The residue was purified by column chromatography (SiO₂, Petroleum ether:Ethyl acetate=10/1 to 2/1) to give the title compound (8 g, 34% yield) as a brown solid. LC-MS (ESI⁺) m/z 449.1 (M+H)⁺.

### Step 3—4′-Chloro-2-(hydroxy (piperidin-4-yl)methyl)-[1,1′-biphenyl]-4-carbonitrile

To a solution of tert-butyl 4-((4′-chloro-4-cyano-[1,1′-biphenyl]-2-yl)(hydroxy)methyl)piperidine-1-carboxylate (8 g, 18.7 mmol) in DCM (80 mL) was added HCl/dioxane (4 M, 4.68 mL). The mixture was then stirred at 25° C. for 1 hr. On completion, the reaction mixture was concentrated under reduced pressure to give the title compound (9 g) as a brown solid. LC-MS (ESI⁺) m/z 327.2 (M+H)⁺.

### Step 4—Methyl 4-(4-((4′-chloro-4-cyano-[1,1′-biphenyl]-2-yl)(hydroxy)methyl)piperidin-1-yl)benzoate

To a solution of 4′-chloro-2-(hydroxy (piperidin-4-yl)methyl)-[1,1′-biphenyl]-4-carbonitrile (9 g, 27.5 mmol) in DMSO (120 mL) was added DIEA (17.8 g, 137 mmol, 23.9 mL) and methyl 4-fluorobenzoate (6.37 g, 41.3 mmol, 5.35 M, CAS #403-33-8). The mixture was stirred at 120° C. for 12 hrs. On completion, the reaction mixture was quenched with water (150 mL) and extracted with ethyl acetate (3× 200 mL). The combined organic layers were washed with brine (300 mL) and dried over anhydrous sodium sulfate, filtered and the filtrate was concentrated in vacuo to give the crude residue. The residue was purified by column chromatography (SiO₂, Petroleum ether/Ethyl acetate=10/1 to 2/1) to give the title compound (2 g, 13% yield) as a brown oil LC-MS (ESI⁺) m/z 461.2 (M+H)⁺.

### Step 5—Methyl 4-(4-((4-(((tert-butoxycarbonyl)amino)methyl)-4′-chloro-[1,1′-biphenyl]-2-yl)(hydroxy)methyl)piperidin-1-yl)benzoate

To a solution of methyl 4-(4-((4′-chloro-4-cyano-[1,1′-biphenyl]-2-yl)(hydroxy)methyl)piperidin-1-yl)benzoate (1.8 g, 3.90 mmol) in THF (30 mL) was added BH₃. THF (1 M, 11.7 mL) dropwise in a three-necked bottle under nitrogen atmosphere. The reaction was stirred at 75° C. for 2 hrs. Then the mixture was allowed to cool to rt and HCl (2 M, 10.8 mL) was added dropwise. The mixture was then stirred at 75° C. for 1 hr. The solvents were then removed under vacuum and residue was suspended in DCM (60 mL). Then Et₃N (908 mg, 8.98 mmol, 1.25 mL) and Boc₂O (2.56 g, 11.7 mmol, 2.69 mL) were added and the reaction was stirred at 25° C. for 1.5 hrs. On completion, the reaction mixture was quenched by water (100 mL) and extracted by dichloromethane (3×150 mL). The combined organic layers were washed by brine (100 mL) and dried over anhydrous sodium sulfate, filtered and the filtrate was concentrated in vacuo to give the crude residue. The residue was purified by column chromatography (SiO₂, Petroleum ether:Ethyl acetate-10/1 to 5/1) to give the title compound (770 mg, 34% yield) as yellow solid. LC-MS (ESI⁺) m/z 565.2 (M+H)⁺.

### Step 6—Methyl (R)-4-(4-((4-(((tert-butoxycarbonyl)amino)methyl)-4′-chloro-[1,1′-biphenyl]-2-yl)(hydroxy)methyl)piperidin-1-yl)benzoate and methyl(S)-4-(4-((4-(((tert-butoxycarbonyl)amino)methyl)-4′-chloro-[1,1′-biphenyl]-2-yl)(hydroxy)methyl)piperidin-1-yl)benzoate

Methyl 4-(4-((4-(((tert-butoxycarbonyl)amino)methyl)-4′-chloro-[1,1′-biphenyl]-2-yl)(hydroxy)methyl)piperidin-1-yl)benzoate was purified by SFC (column: DAICEL CHIRALCEL OX (250 mm*30 mm, 10 um); mobile phase: [Neu-ETOH]; B %: 40%-40%, A4; 60 min) to give methyl (R)-4-(4-((4-(((tert-butoxycarbonyl)amino)methyl)-4′-chloro-[1,1′-biphenyl]-2-yl)(hydroxy)methyl)piperidin-1-yl)benzoate (80 mg, 10% yield) as a pink solid LC-MS (ESI⁺) m/z 565.3 (M+H)⁺ and methyl(S)-4-(4-((4-(((tert-butoxycarbonyl)amino)methyl)-4′-chloro-[1,1′-biphenyl]-2-yl)(hydroxy)methyl)piperidin-1-yl)benzoate (100 mg, 12% yield) as a pink solid. LC-MS (ESI⁺) m/z 565.3 (M+H)⁺ for both isomers. The absolute stereochemistry of the enantiomers was assigned arbitrarily.

### Synthesis of (R)-4-(4-((4-(((tert-butoxycarbonyl)amino)methyl)-4′-chloro-[1,1′-biphenyl]-2-yl)(hydroxy)methyl)piperidin-1-yl)benzoic Acid (Intermediate NC)

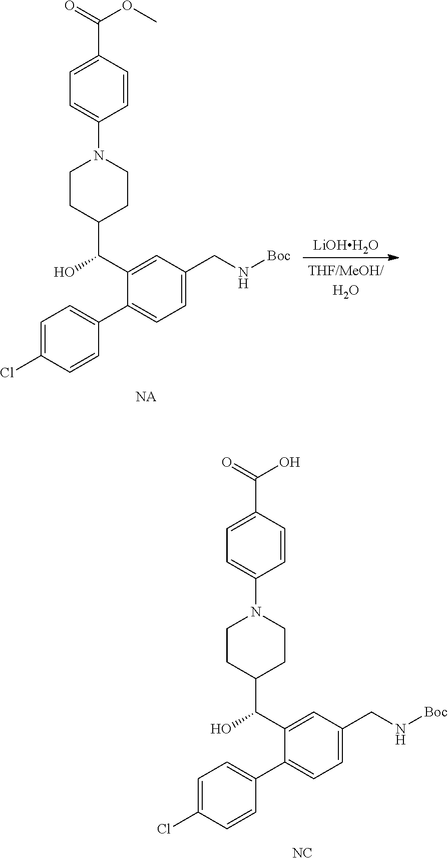

To a solution of methyl (R)-4-(4-((4-(((tert-butoxycarbonyl)amino)methyl)-4′-chloro-[1,1′-biphenyl]-2-yl)(hydroxy)methyl)piperidin-1-yl)benzoate (80 mg, 141 μmol, Intermediate NA) in THF (0.5 mL), MeOH (0.5 mL) and H₂O (0.2 mL) was added LiOH·H₂O (29.7 mg, 707 μmol). The mixture was then stirred at 50° C. for 2 hrs. On completion, HCl (1N) was added to the reaction mixture until the pH was 5, then the mixture was diluted with water (2 mL) and extracted by dichloromethane (3×9 mL), The combined organic layers were dried over anhydrous sodium sulfate, filtered and the filtrate was concentrated in vacuo to give the title compound (80 mg) as a pink solid. LC-MS (ESI+) m/z 551.2 (M+H)+.

### Synthesis of 4-(4-((S)-(4-(aminomethyl)-4′-chloro-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide (Intermediate ND)

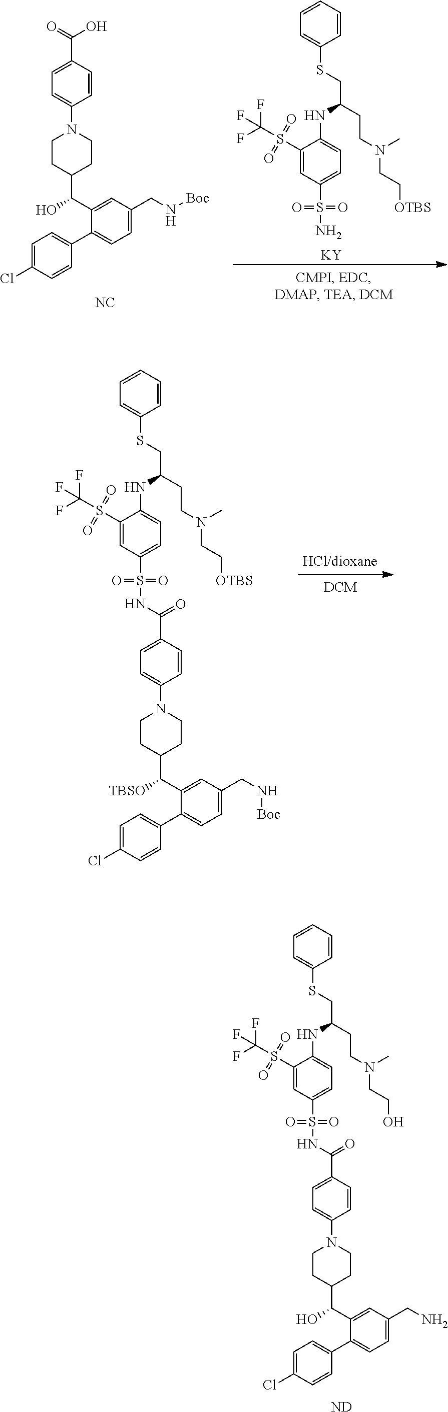

### Step 1—Tert-butyl ((2-((R)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)methyl)carbamate

To a solution of (R)-4-(4-((4-(((tert-butoxycarbonyl)amino)methyl)-4′-chloro-[1,1′-biphenyl]-2-yl)(hydroxy)methyl)piperidin-1-yl)benzoic acid (60 mg, 109 μmol, Intermediate NC) in DCM (4 mL) was added DMAP (13.3 mg, 109 μmol), TEA (16.5 mg, 163 μmol, 22.7 uL) and CMPI (41.7 mg, 163 μmol). Then (R)-4-((4-((2-((tert-butyldimethylsilyl)oxy)ethyl)(methyl)amino)-1-(phenylthio)butan-2-yl)amino)-3-((trifluoromethyl)sulfonyl)benzenesulfonamide (78.5 mg, 119 μmol, Intermediate KY) was added and the mixture was stirred at 25° C. for 1 hr. On completion, the mixture was concentrated to give the crude product. The crude product was purified by reversed-phase HPLC (0.1% FA condition) to give the title compound (90 mg, 63% yield) as a white solid. LC-MS (ESI⁺) m/z 1190.0 (M+H)⁺.

### Step 2—4-(4-((S)-(4-(aminomethyl)-4′-chloro-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide

To a solution of tert-butyl ((2-((R)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)methyl)carbamate (90 mg, 75.7 μmol) in DCM (1 mL) was added HCl/dioxane (4 M, 600 uL). The mixture was then stirred at 25° C. for 2 hrs. On completion, the reaction mixture was concentrated under reduced pressure to give the title compound (90 mg. HCl) as a white solid. LC-MS (ESI+) m/z 974.4 (M+H)⁺.

### Synthesis of (S)-4-(4-((4-(((tert-butoxycarbonyl)amino)methyl)-4′-chloro-[1,1′-biphenyl]-2-yl)(hydroxy)methyl)piperidin-1-yl)benzoic Acid (Intermediate NE)

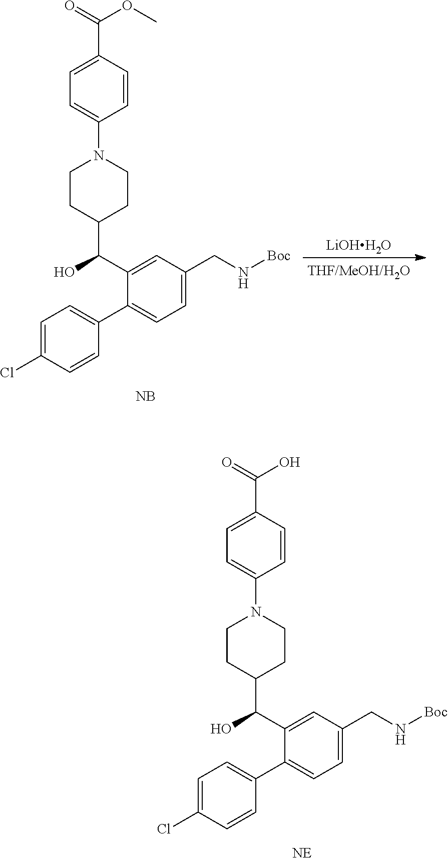

To a solution of methyl 4-[4-[(S)-[5-[(tert-butoxycarbonylamino)methyl]-2-(4-chlorophenyl)phenyl]-hydroxy-methyl]-1-piperidyl]benzoate (80 mg, 141 μmol, Intermediate NB) in THF (0.5 mL), MeOH (0.5 mL) and H₂O (0.2 mL) was added LiOH·H₂O (29.7 mg, 707 μmol). The mixture was then stirred at 50° C. for 2 hrs. On completion, HCl (1N) was added to the reaction mixture until the pH was 5, then the mixture was diluted with water (2 mL) and extracted with dichloromethane (3×9 mL), The combined organic layers were dried over anhydrous sodium sulfate, filtered and the filtrate was concentrated in vacuo to give the title compound (80 mg) as a pink solid. LC-MS (ESI⁺) m/z 551.2 (M+H)⁺.

### Synthesis of 4-(4-((S)-(4-(aminomethyl)-4′-chloro-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide (Intermediate NF)

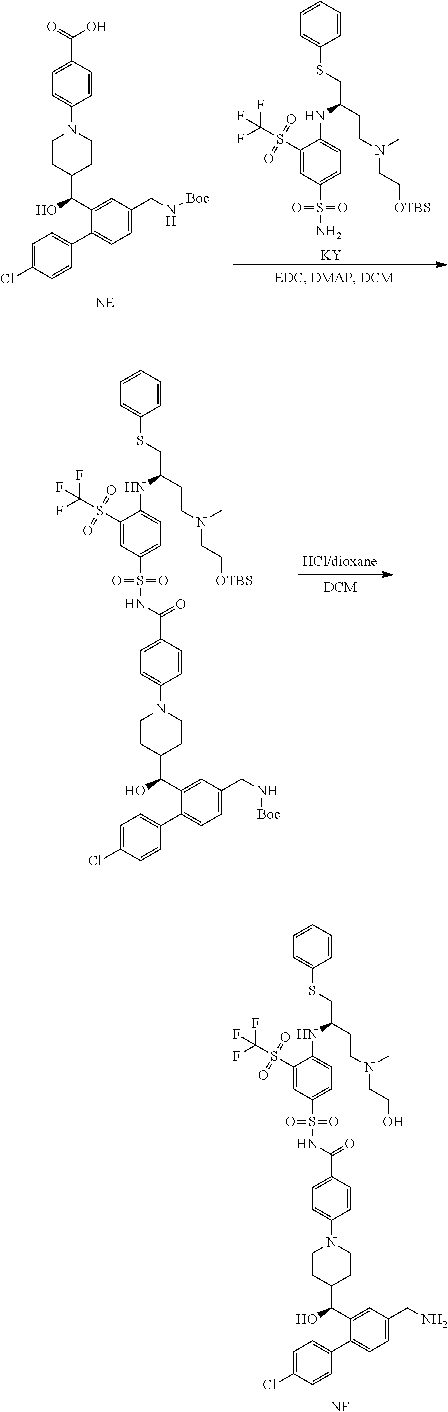

### Step 1—Tert-butyl ((2-((S)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)methyl)carbamate

To a solution of 4-[4-[(S)-[5-[(tert-butoxycarbonylamino)methyl]-2-(4-chlorophenyl)phenyl]-hydroxy-methyl]-1-piperidyl]benzoic acid (60 mg, 109 μmol, Intermediate NE) in DCM (2 mL) was added EDC (50.7 mg, 327 μmol) and DMAP (39.9 mg, 327 μmol). Then 4-[[(1R)-3-[2-[tert-butyl(dimethyl)silyl]oxyethyl-methyl-amino]-1-(phenylsulfanylmethyl)propyl]amino]-3-(trifluoromethylsulfonyl)benzenesulfonamide (71.4 mg, 109 μmol, Intermediate KY) was added and the mixture was stirred at 40° C. for 2 hrs. On completion, the reaction mixture was concentrated under reduced pressure to give a crude product. The crude product was purified by reversed-phase HPLC (0.1% HCl condition) to give the title compound (40 mg, 27% yield) as a pink solid. LC-MS (ESI⁺) m/z 1190.0 (M+H)⁺.

### Step 2—4-(4-((S)-(4-(aminomethyl)-4′-chloro-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide

To a solution of tert-butyl N-[[3-[(S)-[1-[4-[[4-[[(1R)-3-[2-[tert-butyl(dimethyl)silyl]oxyethyl-methyl-amino]-1-(phenylsulfanylmethyl)propyl]amino]-3-(trifluoromethylsulfonyl)phenyl]sulfonylcarbamoyl]phenyl]-4-piperidyl]-hydroxy-methyl]-4-(4-chlorophenyl)phenyl]methyl]carbamate (30 mg, 25.2 μmol) in DCM (1 mL) was added HCl/dioxane (4 M, 600 uL). The mixture was then stirred at 25° C. for 0.5 hrs. On completion, the reaction mixture was concentrated under reduced pressure to give the title compound (30 mg. HCl) as a white solid. LC-MS (ESI⁺) m/z 974.4 (M+H)⁺.

### Synthesis of Ethyl (S)-1-(4-(2-((1-(4-(tert-butoxycarbonyl)phenyl)piperidin-4-yl)((tert-butyldimethylsilyl)oxy)methyl)-4′-chloro-[1,1′-biphenyl]-4-carboxamido)phenyl)piperidine-4-carboxylate (Intermediate NG) and ethyl (R)-1-(4-(2-((1-(4-(tert-butoxycarbonyl)phenyl)piperidin-4-yl) ((tert-butyldimethylsilyl)oxy)methyl)-4′-chloro-[1,1′-biphenyl]-4-carboxamido)phenyl)piperidine-4-carboxylate (Intermediate NH)

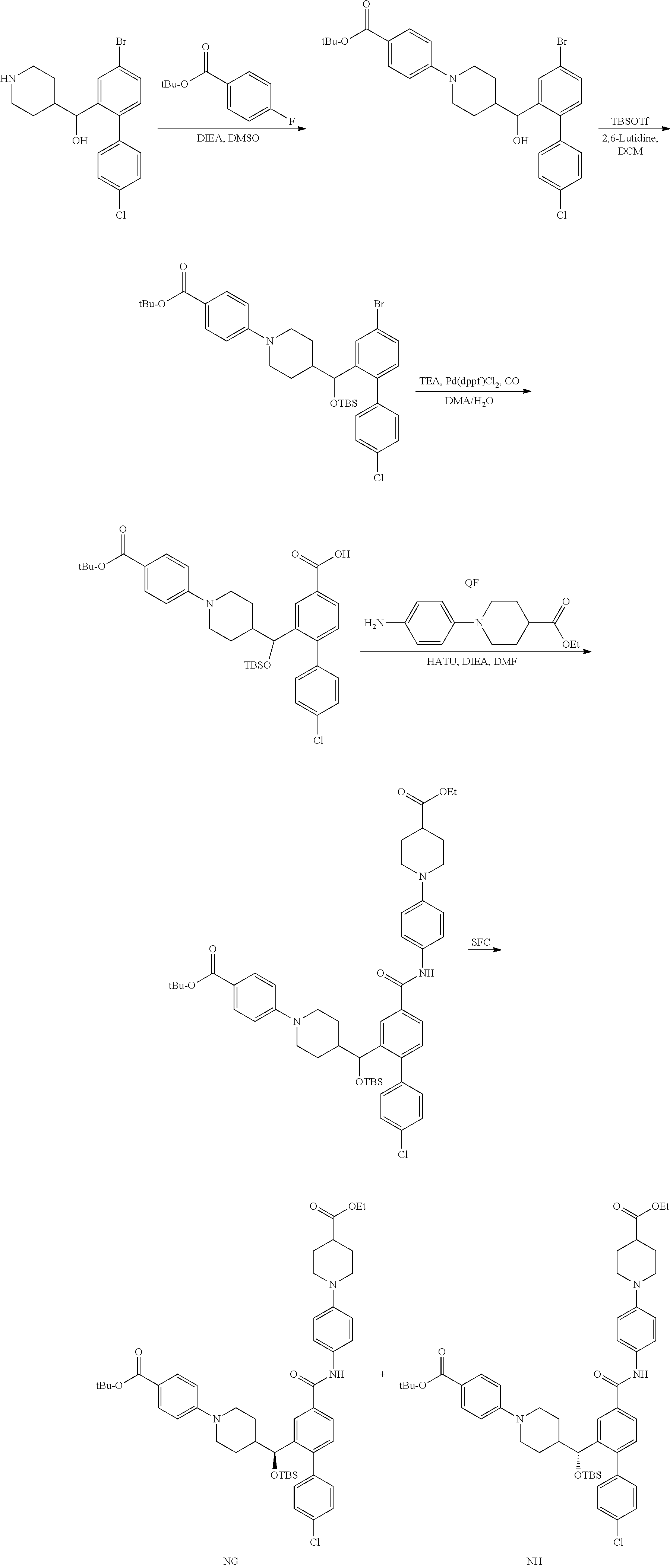

### Step 1—Tert-butyl 4-(4-((4-bromo-4′-chloro-[1,1′-biphenyl]-2-yl)(hydroxy)methyl)piperidin-1-yl)benzoate

To a solution of [5-bromo-2-(4-chlorophenyl)phenyl]-(4-piperidyl) methanol (4.2 g, 10.1 mmol, HCl, synthesized via Steps 1-3 of Intermediate KU) in DMSO (50 mL) was added DIEA (13.0 g, 101 mmol) and tert-butyl 4-fluorobenzoate (3.95 g, 20.14 mmol, CAS #8656-98-7). The mixture was then stirred at 120° C. for 48 hrs. On completion, the mixture was diluted with H₂O (100 mL), and extracted with ethyl acetate (3×100 mL). The combined organic layer was washed with brine (200 mL), dried over anhydrous sodium sulfate, filtered and the filtrate was concentrated in vacuo to give the crude residue. The residue was purified by column chromatography (SiO₂, Petroleum ether/Ethyl acetate=1/0 to 1:1) to give the title compound (1.7 g, 52% yield) as a yellow solid. LC-MS (ESI+) m/z 307.0 (M+H)⁺.

### Step 2—Tert-butyl 4-(4-((4-bromo-4′-chloro-[1,1′-biphenyl]-2-yl)((tert-butyldimethylsilyl)oxy)methyl)piperidin-1-yl)benzoate

To a solution of tert-butyl 4-(4-((4-bromo-4′-chloro-[1,1′-biphenyl]-2-yl)(hydroxy)methyl)piperidin-1-yl)benzoate (3.2 g, 5.75 mmol) in DCM (10 mL) was added TBSCl (1.30 g, 8.62 mmol), TEA (1.74 g, 17.2 mmol) and DMAP (140 mg, 1.15 mmol). The mixture was stirred at 25° C. for 12 hrs. On completion, the mixture was concentrated to give a residue and purified by column chromatography (SiO₂, Petroleum ether/Ethyl acetate=1/0 to 10:1) to give the title compound (1.5 g, 36% yield) as a yellow oil. LC-MS (ESI+) m/z 672.4 (M+H)⁺.

### Step 3—2-((1-(4-(Tert-butoxycarbonyl)phenyl)piperidin-4-yl)(tert-butyldimethylsilyl)oxy)methyl)-4′-chloro-[1,1′-biphenyl]-4-carboxylic Acid

A mixture of 4-(4-((4-bromo-4′-chloro-[1,1′-biphenyl]-2-yl)((tert-tert-butyl butyldimethylsilyl)oxy)methyl)piperidin-1-yl)benzoate (500 mg, 745 μmol), Pd(dppf)Cl₂ (54.5 mg, 74.5 μmol) and TEA (226 mg, 2.23 mmol, 311 uL) in DMA (10 mL) and H₂O (5 mL) then the mixture was stirred at 120° C. for 12 hrs under CO (2.5 MPa) atmosphere. On completion, the mixture was diluted with H₂O (10 mL), and extracted with ethyl acetate (50 mL). The combined organic layer was washed with brine (20 mL), dried over anhydrous sodium sulfate, filtered and the filtrate was concentrated in vacuo to give the crude residue. The residue was purified by column chromatography (SiO₂, Petroleum ether/Ethyl acetate=10/1 to 5:1) to give the title compound (260 mg, 55% yield) as a white solid. LC-MS (ESI+) m/z 636.5 (M+H)⁺.

### Step 4—Ethyl 1-(4-(2-((1-(4-(tert-butoxycarbonyl)phenyl)piperidin-4-yl)((tert-butyldimethylsilyl)oxy)methyl)-4′-chloro-[1,1′-biphenyl]-4-carboxamido)phenyl)piperidine-4-carboxylate

To a solution of 2-((1-(4-(tert-butoxycarbonyl)phenyl)piperidin-4-yl)((tert-butyldimethylsilyl)oxy)methyl)-4′-chloro-[1,1′-biphenyl]-4-carboxylic acid (260 mg, 409 μmol) in DMF (4 ml) was added DIEA (132 mg, 1.02 mmol), HATU (202 mg, 531 μmol) and ethyl 1-(4-aminophenyl)piperidine-4-carboxylate (152 mg, 613 μmol, Intermediate QF). The mixture was then stirred at 25° C. for 4 hrs. On completion, the mixture was diluted with H₂O (10 mL), and extracted with ethyl acetate (5 mL). The combined organic layer was washed with brine (10 mL), dried over anhydrous sodium sulfate, filtered and the filtrate was concentrated in vacuo to give the crude residue. The residue was purified by column chromatography (SiO₂, Petroleum ether/Ethyl acetate=10/1 to 5/1) to give the title compound (360 mg, 99% yield) as a yellow solid. LC-MS (ESI⁺) m/z 866.4 (M+H)⁺.

### Step 5—Ethyl (S)-1-(4-(2-((1-(4-(tert-butoxycarbonyl)phenyl)piperidin-4-yl) (tert-butyldimethylsilyl)oxy)methyl)-4′-chloro-[1,1′-biphenyl]-4-carboxamido)phenyl)piperidine-4-carboxylate and ethyl (R)-1-(4-(2-((1-(4-(tert-butoxycarbonyl)phenyl)piperidin-4-yl)((tert-butyldimethylsilyl)oxy)methyl)-4′-chloro-[1,1′-biphenyl]-4-carboxamido)phenyl)piperidine-4-carboxylate

Ethyl 1-(4-(2-((1-(4-(tert-butoxycarbonyl)phenyl)piperidin-4-yl)((tert-butyldimethyl-silyl)oxy)methyl)-4′-chloro-[1,1′-biphenyl]-4-carboxamido)phenyl)piperidine-4-carboxylate (400 mg) was purified by SFC (column: (s,s) WHELK-O1 (250 mm×30 mm, 5 um); mobile phase: [ACN/MeOH (0.1% NH₃H₂O)]; B %: 50%-50%, A3.9; 80 min) to give ethyl(S)-1-(4-(2-((1-(4-(tert-butoxycarbonyl)phenyl)piperidin-4-yl)((tert-butyldimethylsilyl)oxy)methyl)-4′-chloro-[1,1′-biphenyl]-4-carboxamido)phenyl)piperidine-4-carboxylate (140 mg, 35% yield) and ethyl (R)-1-(4-(2-((1-(4-(tert-butoxycarbonyl)phenyl)piperidin-4-yl)((tert-butyldimethylsilyl)oxy)methyl)-4′-chloro-[1,1′-biphenyl]-4-carboxamido)phenyl)piperidine-4-carboxylate (150 mg, 37% yield) as a yellow solids. LC-MS (ESI⁺) m/z 866.5 (M+H)⁺ for both enantiomers. Absolute stereochemistry of the enantiomers was assigned arbitrarily.

### Synthesis of (S)-4-(4-((4′-chloro-4-((4-(4-(ethoxycarbonyl)piperidin-1-yl)phenyl)carbamoyl)-[1,1′-biphenyl]-2-yl)(hydroxy)methyl)piperidin-1-yl)benzoic Acid (Intermediate NI)

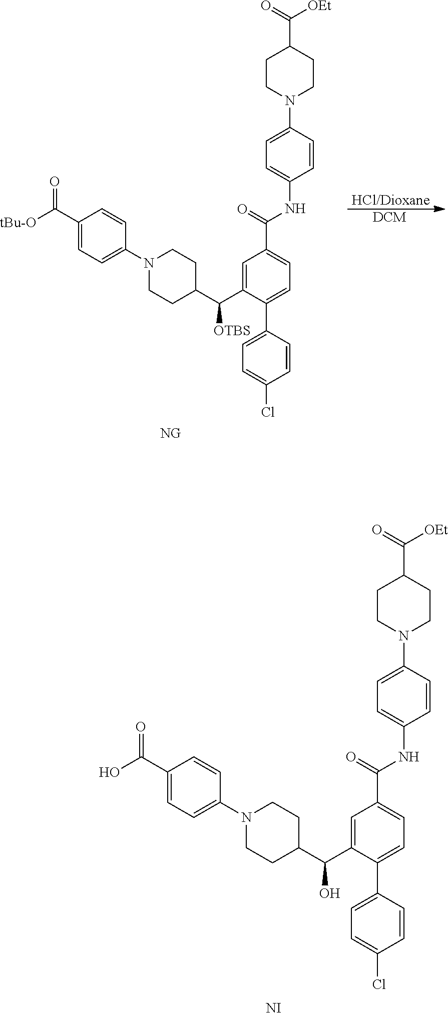

To a solution of ethyl(S)-1-(4-(2-((1-(4-(tert-butoxycarbonyl)phenyl)piperidin-4-yl)((tert-butyldimethylsilyl)oxy)methyl)-4′-chloro-[1,1′-biphenyl]-4-carboxamido)phenyl)piperidine-4-carboxylate (150 mg, 173 μmol, Intermediate NG) in DCM (2 mL) was added HCl/Dioxane (4 M, 1945 uL). The mixture was then stirred at 25° C. for 2 hrs. On completion, the mixture was concentrated to remove organic solvent to give the title compound (120 mg, HCl) as a yellow solid. LC-MS (ESI⁺) m/z 163.2 (M+H)⁺.

### Synthesis of 1-(4-(4′-chloro-2-((S)-hydroxy(1-(4-(((4-(((R)-4-((2-hydroxyethyl)(methyl)amino)-1-(phenylthio)butan-2-yl)amino)-3-((trifluoromethyl)sulfonyl)phenyl)sulfonyl)carbamoyl)phenyl)piperidin-4-yl)methyl)-[1,1′-biphenyl]-4-carboxamido)phenyl)piperidine-4-carboxylic Acid (Intermediate NJ)

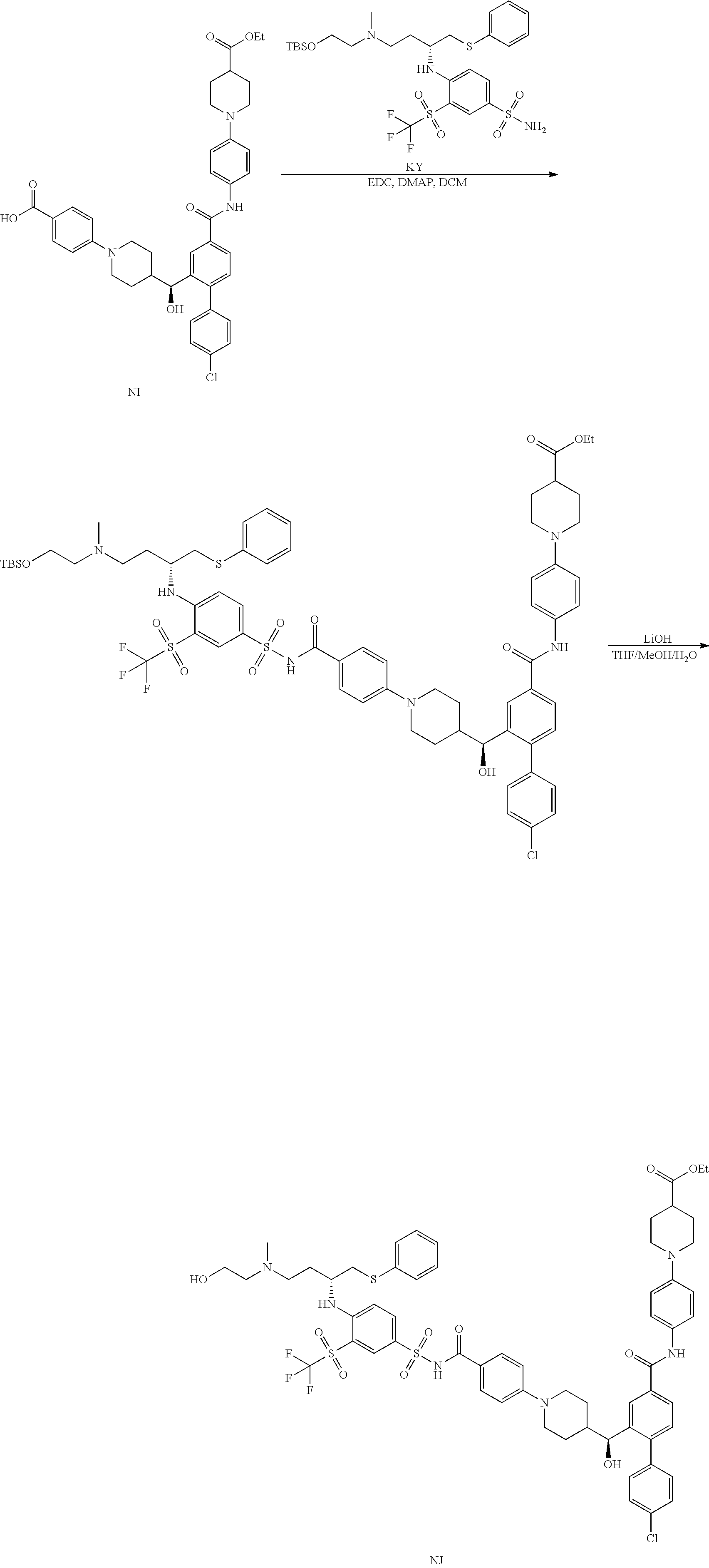

### Step 1—Ethyl 1-(4-(2-((S)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-carboxamido)phenyl)piperidine-4-carboxylate

To a solution of (S)-4-(4-((4′-chloro-4-((4-(4-(ethoxycarbonyl)piperidin-1-yl)phenyl)carbamoyl)-[1,1′-biphenyl]-2-yl)(hydroxy)methyl)piperidin-1-yl)benzoic acid (100 mg, 144 μmol, Intermediate NI) in DCM (4 mL) was added DMAP (52.6 mg, 431 μmol), EDC (44.6 mg, 287 μmol), and (R)-4-((4-((2-((tert-butyldimethylsilyl)oxy)ethyl)(methyl)amino)-1-(phenylthio)butan-2-yl)amino)-3-((trifluoromethyl)sulfonyl)benzenesulfonamide (94.2 mg, 144 μmol, Intermediate KY). The mixture was then stirred at 25° C. for 2 hrs. On completion, the mixture was concentrated to give a residue and purified by reversed-phase HPLC (0.1% FA condition) to give the title compound (90 mg, 31% yield) as a yellow oil. LC-MS (ESI⁺) m/z 1333.4 (M+H)⁺.

### Step 2—1-(4-(4′-Chloro-2-((S)-hydroxy(1-(4-(((4-(((R)-4-((2-hydroxyethyl)(methyl)amino)-1-(phenylthio)butan-2-yl)amino)-3-((trifluoromethyl)sulfonyl)phenyl)sulfonyl)carbamoyl)phenyl)piperidin-4-yl)methyl)-[1,1′-biphenyl]-4-carboxamido)phenyl)piperidine-4-carboxylic Acid

To a solution of ethyl 1-(4-(2-((S)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-carboxamido)phenyl)piperidine-4-carboxylate (40 mg, 30.0 μmol) in THF (2 mL). MeOH (0.5 mL) and H₂O (0.5 mL) was added LiOH·H₂O (3.77 mg, 90.0 μmol). The mixture was then stirred at 40° C. for 12 hrs. On completion, HCl (1N) was added to the reaction mixture until the pH was 6.0, then diluted by water (5 mL) and extracted by dichloromethane (10 mL). The combined organic layers were dried over anhydrous sodium sulfate, filtered and the filtrate was concentrated in vacuo to give the title compound (30 mg) as a white solid. LC-MS (ESI⁺) m/z 596.7 (M/2+H)⁺.

### Synthesis of (R)-4-(4-((4′-chloro-4-((4-(4-(ethoxycarbonyl)piperidin-1-yl)phenyl)carbamoyl)-[1,1′-biphenyl]-2-yl)(hydroxy)methyl)piperidin-1-yl)benzoic Acid (Intermediate NK)

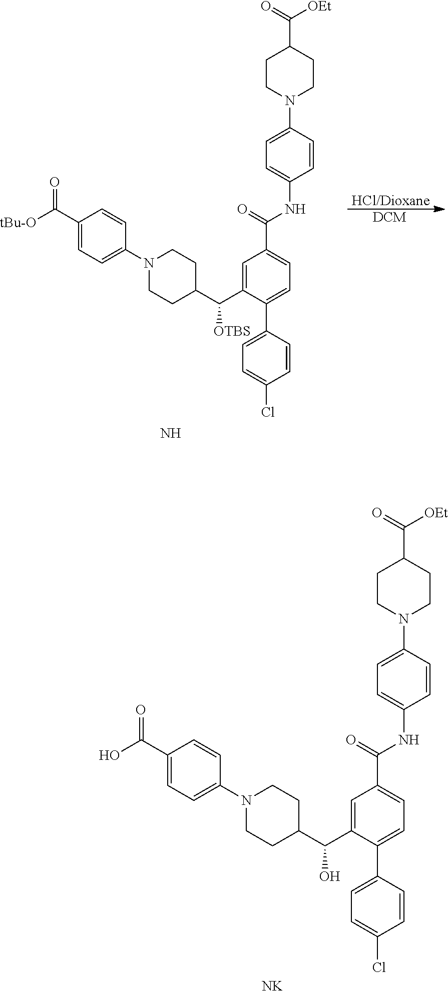

To a solution of ethyl 1-[4-[[3-[(R)-[1-(4-tert-butoxycarbonylphenyl)-4-piperidyl]-[tert-butyl(dimethyl)silyl]oxy-methyl]-4-(4-chlorophenyl)benzoyl]amino]phenyl]piperidine-4-carboxylate (150 mg, 173 μmol, Intermediate NH) in DCM (2 mL) was added HCl/Dioxane (4 M, 194.73 uL). The mixture was then stirred at 25° C. for 2 hrs. On completion, the reaction solution was concentrated to give the title compound (120 mg) as a yellow solid. LC-MS (ESI⁺) m/z 696.5 (M+H)⁺.

### Synthesis of 1-(4-(4′-Chloro-2-((R)-hydroxy(1-(4-(((4-(((R)-4-((2-hydroxyethyl)(methyl)amino)-1-(phenylthio)butan-2-yl)amino)-3-((trifluoromethyl)sulfonyl)phenyl)sulfonyl)carbamoyl)phenyl)piperidin-4-yl)methyl)-[1,1′-biphenyl]-4-carboxamido)phenyl)piperidine-4-carboxylic Acid (Intermediate NL)

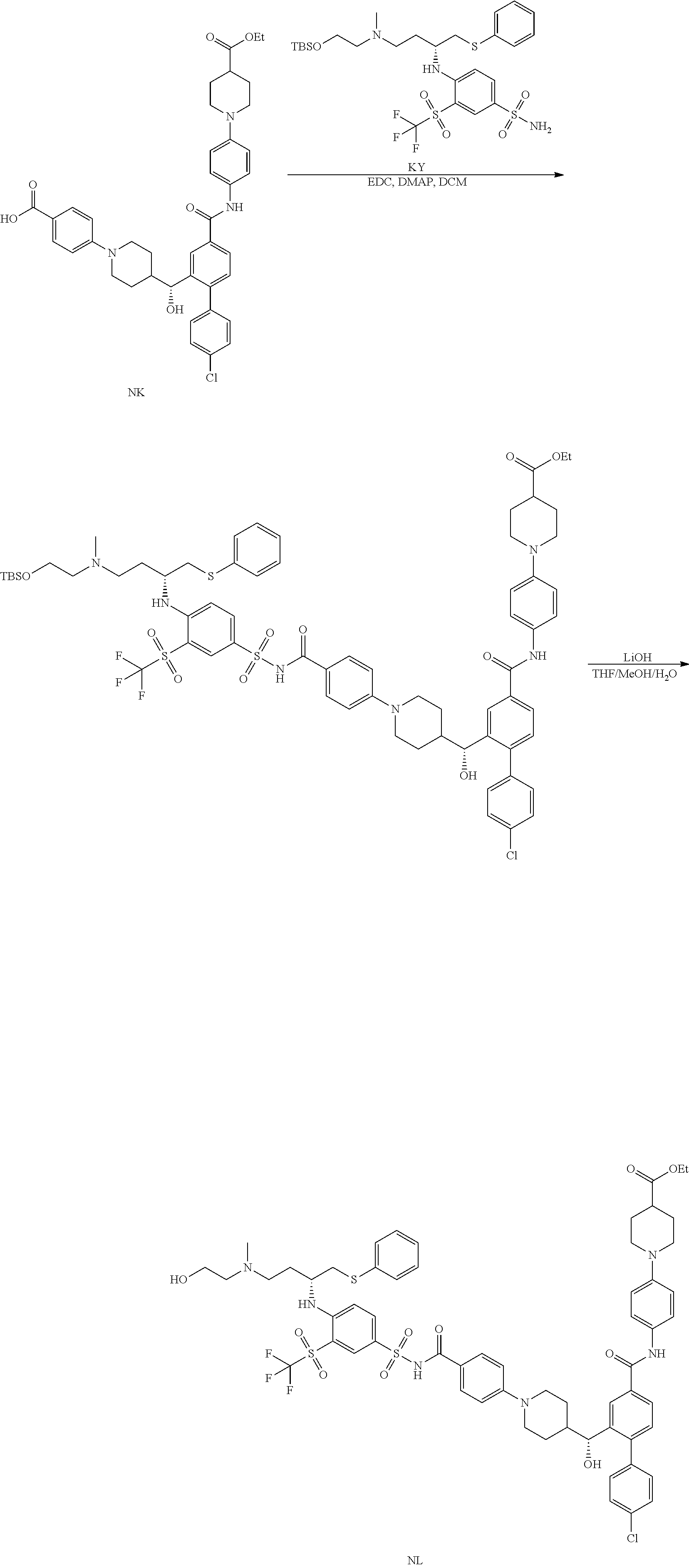

### Step 1—Ethyl 1-(4-(2-((R)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl) sulfonyl)carbamoyl)phenyl)piperidin-4-yl)(hydroxy)methyl)-4′-chloro-[1,1′-biphenyl]-4-carboxamido)phenyl)piperidine-4-carboxylate

To a solution of (R)-4-(4-((4′-chloro-4-((4-(4-(ethoxycarbonyl)piperidin-1-yl)phenyl)carbamoyl)-[1,1′-biphenyl]-2-yl)(hydroxy)methyl)piperidin-1-yl)benzoic acid (120 mg, 172 μmol, Intermediate NK) in DCM (4 mL) was added DMAP (63.2 mg, 517 μmol) EDC (53.5 mg, 345 μmol) and (R)-4-((4-((2-((tert-butyldimethylsilyl)oxy)ethyl)(methyl)amino)-1-(phenylthio)butan-2-yl)amino)-3-((trifluoromethyl)sulfonyl)benzenesulfonamide (113 mg, 172 μmol, Intermediate KY). The mixture was then stirred at 25° C. for 2 hrs. On completion, the mixture was concentrated to give a residue and purified by reversed-phase HPLC (0.1% FA condition) to give the title compound (90 mg, 38% yield) as a yellow oil. LC-MS (ESI+) m/z 667.8 (M/2+H)⁺.

### Step 2—1-(4-(4′-Chloro-2-((R)-hydroxy(1-(4-(((4-(((R)-4-((2-hydroxyethyl)(methyl)amino)-1-(phenylthio)butan-2-yl)amino)-3-((trifluoromethyl)sulfonyl)phenyl)sulfonyl)carbamoyl)phenyl)piperidin-4-yl)methyl)-[1,1′-biphenyl]-4-carboxamido)phenyl)piperidine-4-carboxylic Acid

To a solution of ethyl 1-(4-(2-((R)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-carboxamido)phenyl)piperidine-4-carboxylate (40 mg, 30.0 μmol) in THF (2 mL), MeOH (0.5 mL) and H₂O (0.5 mL) was added LiOH·H₂O (3.77 mg, 90.0 μmol). The mixture was then stirred at 40° C. for 12 hrs. On completion, to the reaction mixture was added HCl (1N) until the pH was 6.0, then diluted by water (5 mL) and extracted with dichloromethane (10 mL). The combined organic layers were dried over anhydrous sodium sulfate, filtered and the filtrate was concentrated in vacuo to give the title compound (30 mg) as a white solid. LC-MS (ESI+) m/z 1191.4 (M+H)⁺.

### Synthesis of 4-(4-((4-(((tert-butoxycarbonyl)amino)methyl)-4′-chloro-[1,1′-biphenyl]-2-yl)methyl)piperazin-1-yl)benzoic Acid (Intermediate NM)

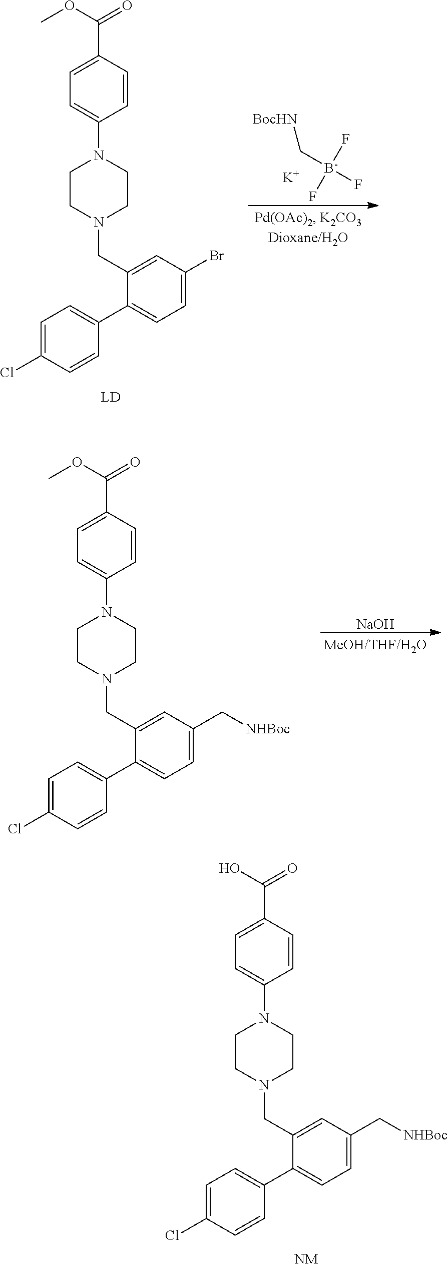 [SMILES: COC(=O)c1ccc(N2CCN(Cc3cc(Br)ccc3-c3ccc(Cl)cc3)CC2)cc1 ; [K+] ; CC(C)(C)OC(=O)NC[B-](F)(F)F ; COC(=O)c1ccc(N2CCN(Cc3cc(CNC(=O)OC(C)(C)C)ccc3-c3ccc(Cl)cc3)CC2)cc1 ; "NaOH" ; CC(C)(C)OC(=O)NCc1ccc(-c2ccc(Cl)cc2)c(CN2CCN(c3ccc(C(=O)O)cc3)CC2)c1]

### Step 1—Methyl 4-(4-((4-(((tert-butoxycarbonyl)amino)methyl)-4′-chloro-[1,1′-biphenyl]-2-yl)methyl)piperazin-1-yl)benzoate

A mixture of methyl 4-(4-((4-bromo-4′-chloro-[1,1′-biphenyl]-2-yl)methyl)piperazin-1-yl)benzoate (2 g, 2.40 mmol, Intermediate LD), potassium (((tert-butoxycarbonyl)amino)methyl)trifluoroborate (740 mg, 3.12 mmol, CAS #1314538-55-0), Pd(OAc)₂ (53.9 mg, 240 μmol), bis(1-adamantyl)-butyl-phosphane (172 mg, 480 μmol) and K₂CO₃ (830 mg, 6.00 mmol) in dioxane (15 mL) and H₂O (5 mL) was degassed and purged with N₂ three times. Then the mixture was stirred at 80° C. for 20 hrs under N₂ atmosphere. On completion, the reaction mixture was quenched/diluted with water (50 mL) and extracted by ethyl acetate (3× 40 mL). The combined organic layers were washed with brine (120 mL) and dried over anhydrous sodium sulfate, filtered and the filtrate was concentrated in vacuo to give the crude residue. The residue was purified by column chromatography (SiO₂, Petroleum ether/Ethyl acetate=10/1 to 3/1) to give the title compound (860 mg, 60% yield) as a white solid. LC-MS (ESI⁺) m/z 550.3 (M+H)⁺.

### Step 2—4-(4-((4-(((Tert-butoxycarbonyl)amino)methyl)-4′-chloro-[1,1′-biphenyl]-2-yl)methyl)piperazin-1-yl)benzoic Acid

To a solution of methyl 4-(4-((4-(((tert-butoxycarbonyl)amino)methyl)-4′-chloro-[1,1′-biphenyl]-2-yl)methyl)piperazin-1-yl)benzoate (850 mg, 1.55 mmol) in THF (8 mL), MeOH (8 mL) and H₂O (4 mL) was added NaOH (124 mg, 3.09 mmol). The mixture was then stirred at 50° C. for 12 hrs. On completion, HCl (1N) was added to the reaction mixture until the pH was 4.0, then diluted by water (10 mL) and extracted by dichloromethane (3×30 mL). The combined organic layers were dried over anhydrous sodium sulfate, filtered and the filtrate was concentrated in vacuo to give the title compound (750 mg) as a gray solid. LC-MS (ESI⁺) m/z 536.2 (M+H)⁺.

### Synthesis of (R)-4-(4-((4-(aminomethyl)-4′-chloro-[1,1′-biphenyl]-2-yl)methyl)piperazin-1-yl)-N-((4-((4-(dimethylamino)-1-(phenylthio)butan-2-yl)amino)-3-nitrophenyl) sulfonyl)benzamide (Intermediate NN)

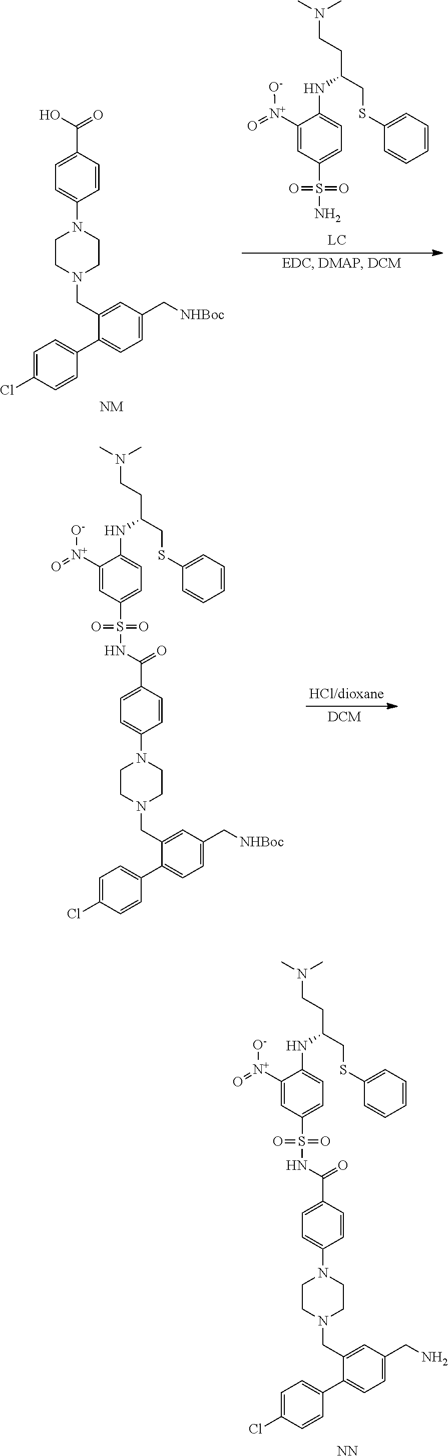

### Step 1—(R)-tert-butyl ((4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)methyl)carbamate

To a solution of 4-(4-((4-(((tert-butoxycarbonyl)amino)methyl)-4′-chloro-[1,1′-biphenyl]-2-yl)methyl)piperazin-1-yl)benzoic acid (152 mg, 283 μmol, Intermediate NM) in DCM (10 mL) was added EDC (73.1 mg, 471 μmol), DMAP (71.9 mg, 589 μmol) and (R)-4-((4-(dimethylamino)-1-(phenylthio)butan-2-yl)amino)-3-nitrobenzenesulfonamide (100 mg, 236 μmol, Intermediate LC). The mixture was then stirred at 25° C. for 3 hrs. On completion, the mixture was concentrated to give the crude residue and purified by reversed-phase HPLC (0.1% FA condition) to give the title compound (160 mg, 68% yield) as a yellow solid. LC-MS (ESI⁺) m/z 942.3 (M+H)⁺.

### Step 2—(R)-4-(4-((4-(aminomethyl)-4′-chloro-[1,1′-biphenyl]-2-yl)methyl)piperazin-1-yl)-N-((4-((4-(dimethylamino)-1-(phenylthio)butan-2-yl)amino)-3-nitrophenyl)sulfonyl)benzamide

To a solution of (R)-tert-butyl ((4′-chloro-2-((4-(4-(((4-((4-(dimethylamino)-1-(phenylthio)butan-2-yl)amino)-3-nitrophenyl)sulfonyl)carbamoyl)phenyl)piperazin-1-yl)methyl)-[1,1′-biphenyl]-4-yl)methyl)carbamate (130 mg, 138 μmol) in DCM (2.5 mL) was added TFA (5.01 g, 43.9 mmol). The mixture was then stirred at 20° C. for 1 hr. On completion, the mixture was concentrated to give the title compound (120 mg, HCl) as a yellow solid. LC-MS (ESI⁺) m/z 842.2 (M+H)⁺.

### Synthesis of 4-(5-Methylhexahydropyrrolo[3,4-c]pyrrol-2 (1H)-yl)benzoic Acid (Intermediate NO)

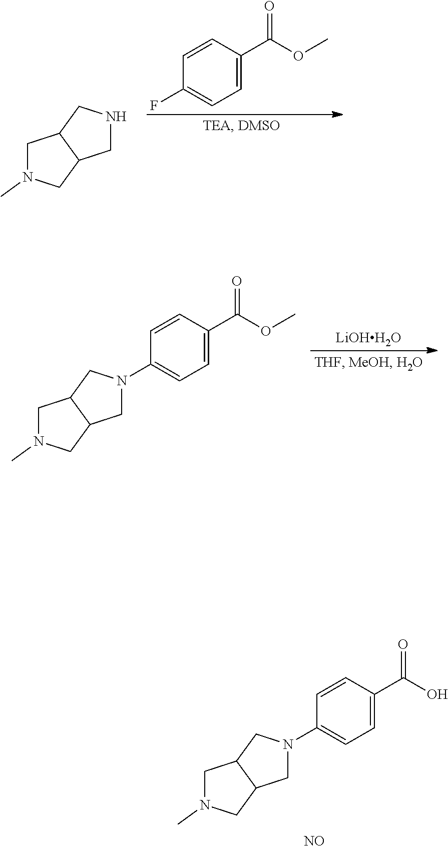

### Step 1—Methyl 4-(5-methylhexahydropyrrolo[3,4-c]pyrrol-2 (1H)-yl)benzoate

To a solution of methyl 4-fluorobenzoate (611 mg, 3.96 mmol, CAS #403-33-8) in DMSO (6 mL) was added 2-methyloctahydropyrrolo[3,4-c]pyrrole (500 mg, 3.96 mmol, CAS #86732-28-7) and TEA (2.00 g, 19.81 mmol). The mixture was then stirred at 120° C. for 16 hrs. On completion, the reaction mixture was diluted with water (20 mL) and extracted by ethyl acetate (3×20 mL). The combined organic layers were washed with brine (60 mL) and dried over anhydrous sodium sulfate, filtered and the filtrate was concentrated in vacuo to give the crude residue. Then the residue was purified by column chromatography (SiO₂, Dichloromethan:Methanol=10/1 to 8/1) to give the title compound (700 mg, 54% yield) as a red solid. LC-MS (ESI⁺) m/z 261.1 (M+H)⁺.

### Step 2—4-(5-Methylhexahydropyrrolo[3,4-c]pyrrol-2 (1H)-yl)benzoic Acid

To a solution of methyl 4-(5-methylhexahydropyrrolo[3,4-c]pyrrol-2 (1H)-yl)benzoate (500 mg, 1.92 mmol) in THF (2 mL), MeOH (2 mL) and H₂O (0.8 mL) was added LiOH·H₂O (806 mg, 19.2 mmol). The mixture was then stirred at 50° C. for 12 hrs. On completion, the reaction mixture was concentrated in vacuo to remove the solvent. The crude residue was purified by reversed-phase HPLC (0.1% HCl condition) to give the title compound (200 mg, 41% yield, HCl) as a pink solid. LC-MS (ESI⁺) m/z 247.2 (M+H)⁺.

### Synthesis of 4-(5-Methylhexahydropyrrolo[3,4-c]pyrrol-2 (1H)-yl)-N-((4-(((R)-1-(phenylthio)-4-(piperazin-1-yl)butan-2-yl)amino)-3-((trifluoromethyl)sulfonyl)phenyl)sulfonyl)benzamide (Intermediate NP)

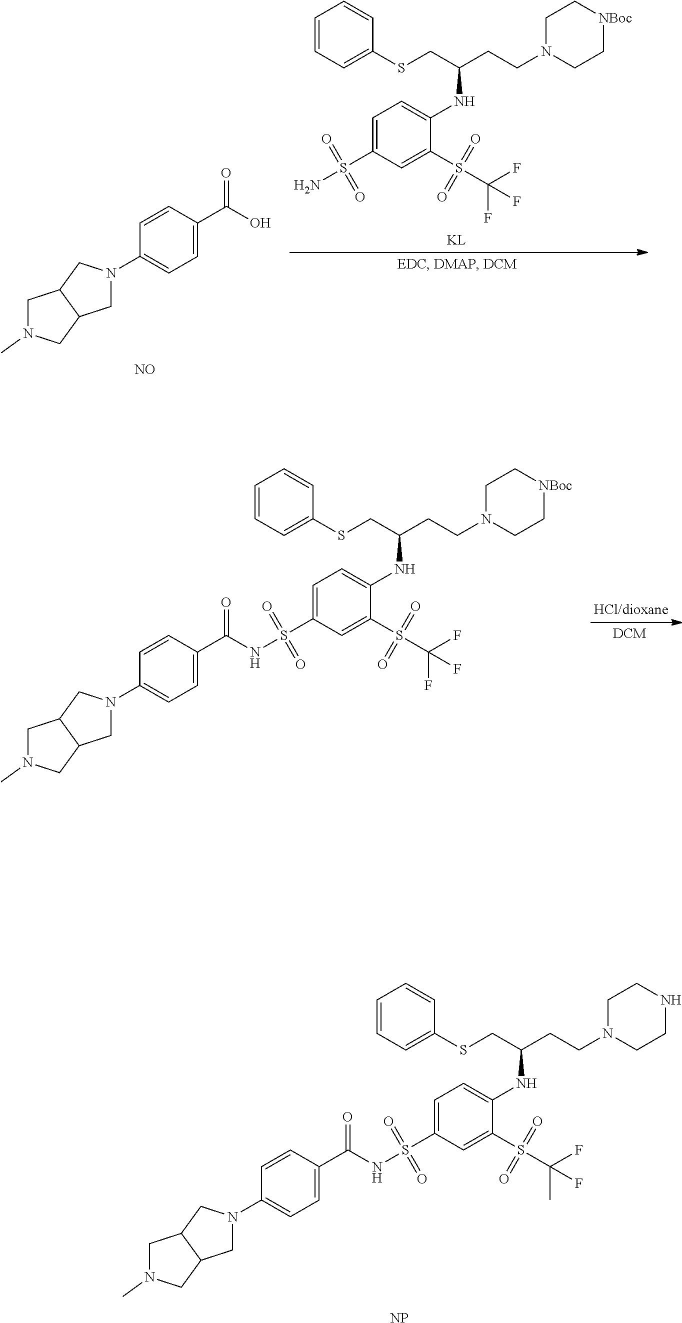 [SMILES: CN1CC2CN(c3ccc(C(=O)O)cc3)CC2C1 ; CC(C)(C)OC(=O)N1CCN(CC[C@H](CSc2ccccc2)Nc2ccc(S(N)(=O)=O)cc2S(=O)(=O)C(F)(F)F)CC1 ; "NO" ; CN1CC2CN(c3ccc(C(=O)NS(=O)(=O)c4ccc(N[C@H](CCN5CCN(C(=O)OC(C)(C)C)CC5)CSc5ccccc5)c(S(=O)(=O)C(F)(F)F)c4)cc3)CC2C1 ; CN1CC2CN(c3ccc(C(=O)NS(=O)(=O)c4ccc(N[C@H](CCN5CCNCC5)CSc5ccccc5)c(S(=O)(=O)C(C)(F)F)c4)cc3)CC2C1 ; "NP"]

### Step 1—Tert-butyl 4-((3R)-3-((4-(N-(4-(5-methylhexahydropyrrolo[3,4-c]pyrrol-2 (1H)-yl)benzoyl)sulfamoyl)-2-((trifluoromethyl)sulfonyl)phenyl)amino)-4-(phenylthio)butyl)piperazine-1-carboxylate

To a solution of 4-(5-methylhexahydropyrrolo[3,4-c]pyrrol-2 (1H)-yl)benzoic acid (43.3 mg, 153 μmol, HCl, Intermediate NO) in DCM (4 mL) was added DMAP (46.8 mg, 383 μmol), EDC (47.6 mg, 306 μmol) and (R)-tert-butyl 4-(4-(phenylthio)-3-((4-sulfamoyl-2-((trifluoromethyl)sulfonyl)phenyl)amino)butyl)piperazine-1-carboxylate (100 mg, 153 μmol, Intermediate KL). Then the mixture was stirred at 25° C. for 6 hrs. On completion, the reaction mixture was concentrated in vacuo to remove the solvent. Then the crude residue was purified by reversed-phase HPLC (0.1% ammonium hydroxide) to give the title compound (120 mg, 88% yield) as a white solid. LC-MS (ESI⁺) m/z 881.8 (M+H)⁺.

### Step 2—4-(5-Methylhexahydropyrrolo[3,4-c]pyrrol-2 (1H)-yl)-N-((4-(((R)-1-(phenylthio)-4-(piperazin-1-yl)butan-2-yl)amino)-3-((trifluoromethyl)sulfonyl)phenyl)sulfonyl)benzamide

To a solution of tert-butyl 4-((3R)-3-((4-(N-(4-(5-methylhexahydropyrrolo[3,4-c]pyrrol-2(1H)-yl)benzoyl)sulfamoyl)-2-((trifluoromethyl)sulfonyl)phenyl)amino)-4-(phenylthio)butyl)piperazine-1-carboxylate (40 mg, 45.4 μmol) in DCM (4 mL) was added HCl/dioxane (4 M, 0.8 mL). The mixture was then stirred at 25° C. for 1 hr. On completion, the reaction mixture was concentrated in vacuo to give title compound (32 mg, HCl) as a white solid. LC-MS (ESI⁺) m/z 781.8 (M+H)⁺.

### Synthesis of 2-((1H-pyrrolo[2,3-b]pyridin-5-yl)oxy)-4-(4-((4′-chloro-[1,1′-biphenyl]-2-yl)methyl)piperazin-1-yl)benzoic Acid (Intermediate NO)

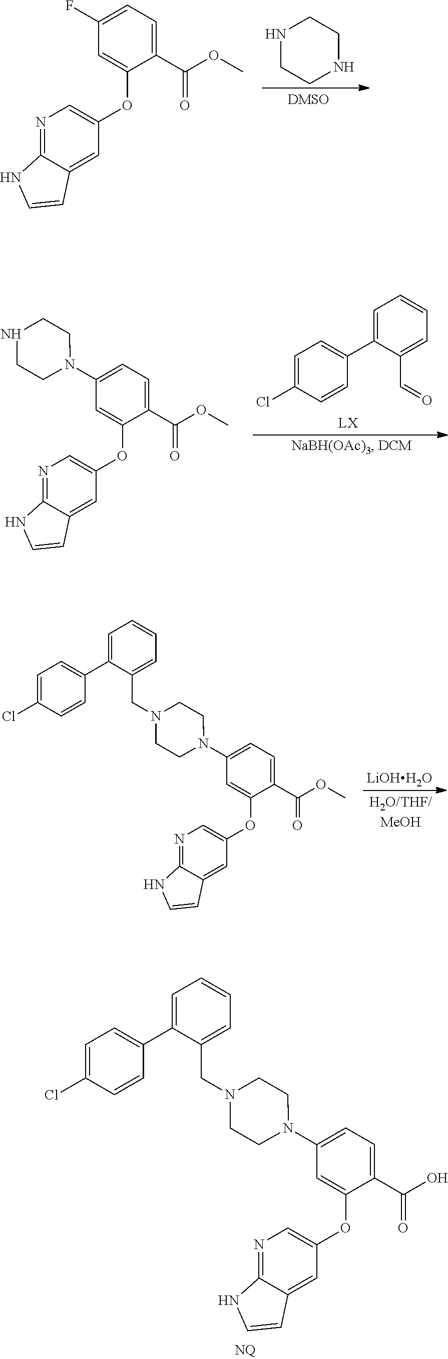

### Step 1—Methyl 2-((1H-pyrrolo[2,3-b]pyridin-5-yl)oxy)-4-(piperazin-1-yl)benzoate

To a solution of methyl 2-((1H-pyrrolo[2,3-b]pyridin-5-yl)oxy)-4-fluorobenzoate (2 g, 6.99 mmol, CAS #1235865-75-4) in DMSO (20 mL) was added piperazine (722 mg, 8.38 mmol, CAS #110-85-0), then the reaction was stirred at 50° C. for 16 hrs. On completion, the reaction was diluted by water (10 mL). Then the mixture was filtered and the filter cake was purified by reversed-phase HPLC (0.1% FA condition) to give the title compound (1.8 g, 64% yield, FA) as a white solid. LC-MS (ESI⁺) m/z 353.2 (M+H)⁺.

### Step 2—Methyl 2-((1H-pyrrolo[2,3-b]pyridin-5-yl)oxy)-4-(4-((4′-chloro-[1,1′-biphenyl]-2-yl)methyl)piperazin-1-yl)benzoate

To a solution of methyl 2-((1H-pyrrolo[2,3-b]pyridin-5-yl)oxy)-4-(piperazin-1-yl)benzoate (500 mg, 1.25 mmol, FA) and 4′-chloro-[1,1′-biphenyl]-2-carbaldehyde (271 mg, 1.25 mmol, Intermediate LX) in DCM (10 mL) was added NaBH(OAc)₃ (265 mg, 1.25 mmol). The mixture was then stirred at 40° C. for 12 hrs. On completion, the reaction mixture was quenched with water (2 mL) and extracted by dichloromethane (2×5 mL). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 1/1) to give the title compound (500 mg, 71% yield) as a colorless oil. LC-MS (ESI⁺) m/z 553.4 (M+H)⁺.

### Step 3—2-((1H-pyrrolo[2,3-b]pyridin-5-yl)oxy)-4-(4-((4′-chloro-[1,1′-biphenyl]-2-yl)methyl)piperazin-1-yl)benzoic Acid

To a solution of methyl 2-((1H-pyrrolo[2,3-b]pyridin-5-yl)oxy)-4-(4-((4′-chloro-[1,1′-biphenyl]-2-yl)methyl)piperazin-1-yl)benzoate (500 mg, 904 μmol) in THF (4 mL), H₂O (2 mL) and MeOH (2 mL) was added LiOH·H₂O (189 mg, 4.52 mmol). The mixture was then stirred at 50° C. for 12 hrs. On completion, HCl (1N) was added to the reaction mixture until the pH was 6.0, then diluted by water (5 mL) and extracted by dichloromethane (2×10 mL). The combined organic layers were dried over anhydrous sodium sulfate, filtered and the filtrate was concentrated in vacuo to give the title compound (450 mg) as a white solid LC-MS (ESI⁺) m/z 539.5 (M+H)⁺.

### Synthesis of 2-((1H-pyrrolo[2,3-b]pyridin-5-yl)oxy)-4-(4-((4′-chloro-[1,1′-biphenyl]-2-yl)methyl)piperazin-1-yl)-N-((4-((3-(piperazin-1-yl)propyl)amino)-3-((trifluoromethyl)sulfonyl)phenyl)sulfonyl)benzamide (Intermediate NR)

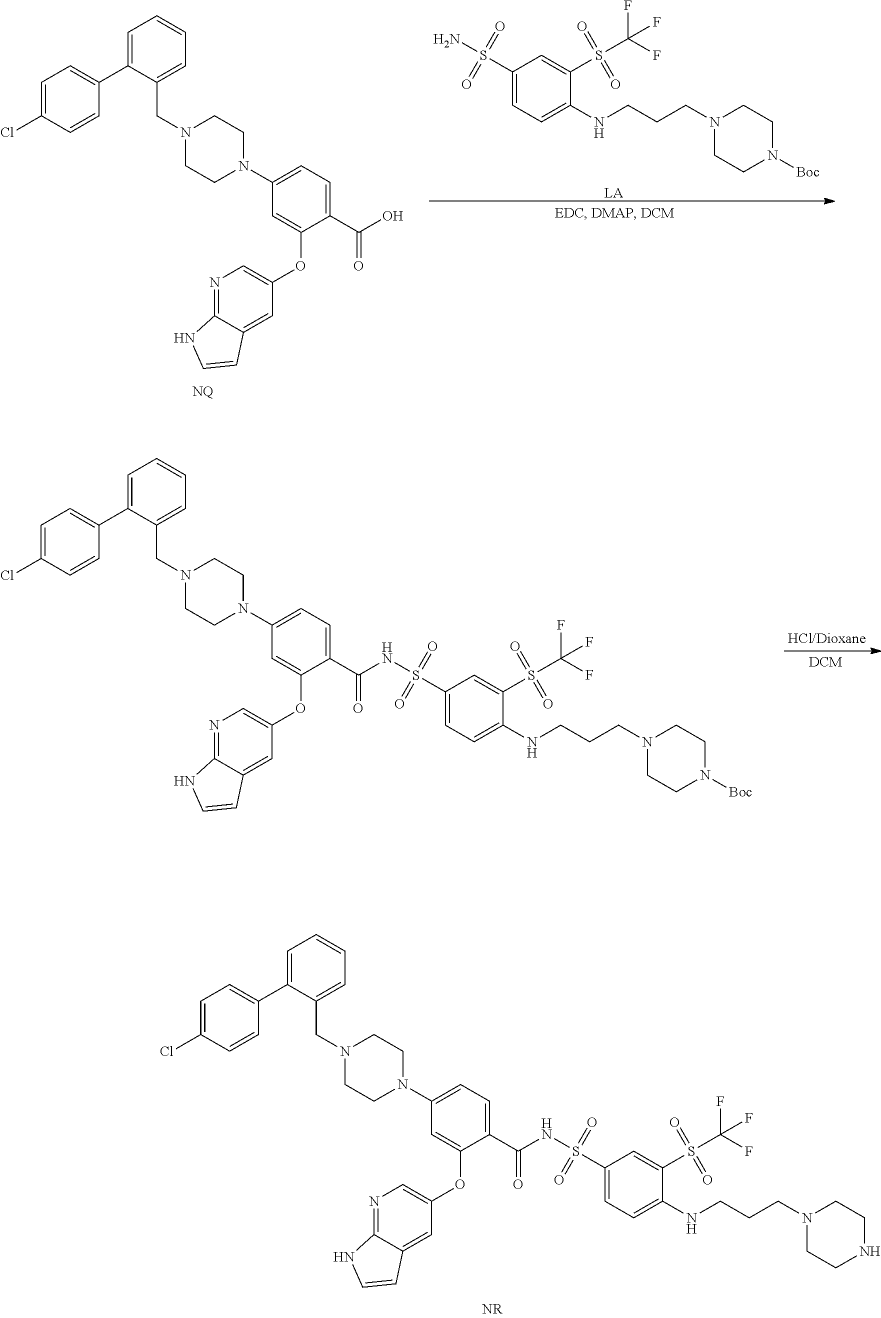

### Step 1—Tert-butyl 4-(3-((4-(N-(2-((1H-pyrrolo[2,3-b]pyridin-5-yl)oxy)-4-(4-((4′-chloro-[1,1′-biphenyl]-2-yl)methyl)piperazin-1-yl)benzoyl)sulfamoyl)-2-((trifluoromethyl)sulfonyl)phenyl)amino)propyl)piperazine-1-carboxylate

To a solution of 2-((1H-pyrrolo[2,3-b]pyridin-5-yl)oxy)-4-(4-((4′-chloro-[1,1′-biphenyl]-2-yl)methyl)piperazin-1-yl)benzoic acid (300 mg, 556 μmol, Intermediate NQ) in DCM (2 mL) was added EDC (172 mg, 1.11 mmol, 197 uL). Then the mixture was stirred for 30 min, and tert-butyl 4-(3-((4-sulfamoyl-2-((trifluoromethyl)sulfonyl)phenyl)amino)propyl)piperazine-1-carboxylate (295 mg, 556 μmol, Intermediate LA) and DMAP (169 mg, 1.39 mmol) was added. Then the mixture was stirred at 25° C. for 3 hrs. On completion, the reaction mixture was concentrated under reduced pressure to remove solvent and purified by prep-HPLC (0.8 g/L ammonium bicarbonate) to give the title compound (250 mg, 40% yield) as a white solid. LC-MS (ESI⁺) m/z 1052.3 (M+H)⁺.

### Step 2—2-((1H-pyrrolo[2,3-b]pyridin-5-yl)oxy)-4-(4-((4′-chloro-[1,1′-biphenyl]-2-yl)methyl)piperazin-1-yl)-N-((4-((3-(piperazin-1-yl)propyl)amino)-3-((trifluoromethyl)sulfonyl)phenyl)sulfonyl)benzamide

To a solution of tert-butyl 4-(3-((4-(N-(2-((1H-pyrrolo[2,3-b]pyridin-5-yl)oxy)-4-(4-((4′-chloro-[1,1′-biphenyl]-2-yl)methyl)piperazin-1-yl)benzoyl)sulfamoyl)-2-((trifluoromethyl)sulfonyl)phenyl)amino)propyl)piperazine-1-carboxylate (250 mg, 223 μmol) in DCM (10 mL) was added HCl/dioxane (4 M, 1 mL). The mixture was stirred at 25° C. for 1 hr. On completion, the reaction mixture was concentrated under reduced pressure to remove solvent to give the title compound (200 mg, 91% yield, HCl) as a white solid. LC-MS (ESI⁺) m/z 951.2 (M+H)⁺.

### Synthesis of 4-((3-Morpholinopropyl)amino)-3-(trifluoromethyl)sulfonyl)benzenesulfonamide (Intermediate NS)

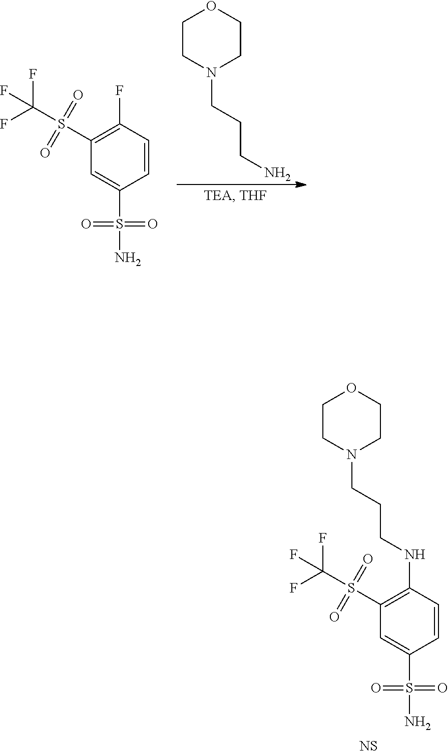 [SMILES: NS(=O)(=O)c1ccc(F)c(S(=O)(=O)C(F)(F)F)c1 ; NCCCN1CCOCC1 ; NS(=O)(=O)c1ccc(NCCCN2CCOCC2)c(S(=O)(=O)C(F)(F)F)c1 ; "NS"]

To a solution of 3-morpholinopropan-1-amine (1 g, 6.93 mmol, CAS #123-00-2) in THF (20 mL) was added TEA (2.10 g, 20.8 mmol) and 4-fluoro-3-((trifluoromethyl)sulfonyl)benzenesulfonamide (2.13 g, 6.93 mmol, CAS #1027345-08-9). The mixture was then stirred at 60° C. for 4 hrs. On completion, the mixture was diluted with H₂O (50 mL), and extracted with EtOAc (30 mL×3). The combined organic layer was washed with brine (20 mL×3), dried over anhydrous Na₂SO₄, filtered and concentrated to give the title compound (3 g) as a white solid. LC-MS (ESI⁺) m/z 432.3 (M+H)⁺.

### Synthesis of (R)-(4′-chloro-6-(chloromethyl)-4-methyl-2,3,4,5-tetrahydro-[1,1′-biphenyl]-4-yl) methanol (Intermediate NT)

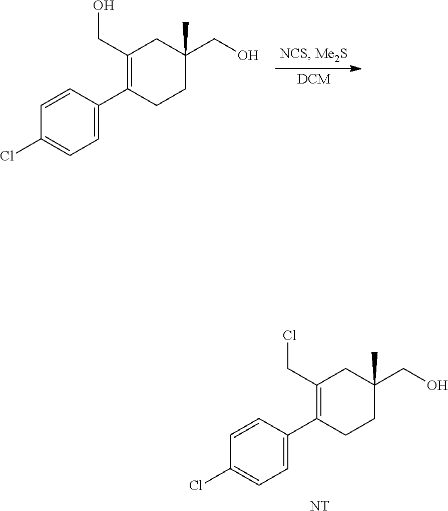

To a solution of (R)-(4′-chloro-4-methyl-3,4,5,6-tetrahydro-[1,1′-biphenyl]-2,4-diyl)dimethanol (5 g, 18.7 mmol, CAS #2471970-48-4) in DCM (50 mL) was added NCS (2.88 g, 21.6 mmol) and then Me₂S (1.34 g, 21.6 mmol) was added dropwise at −30° C. The mixture was stirred at 0° C. for 1 hr. On completion, the mixture was quenched with water (60 mL), and extracted with DCM (60 mL×3). The combined organic layers were washed with brine (10 mL×3), dried over Na₂SO₄ and evaporated. The water phase was quenched with NaClO solution (10 mL) and then discarded. The residue was purified by column chromatography (SiO₂, Petroleum ether/Ethyl acetate=10/1 to 2/1) to give the title compound (6 g, 65% yield) as a colorless oil. ¹H NMR (400 MHz, CHLOROFORM-d) δ ppm 7.25 (d, J=8.4 Hz, 2H), 7.06-7.10 (m, 2H), 3.82 (s, 2H), 3.39 (s, 2H), 2.25 (br t, J=6.4 Hz, 2H), 1.52-1.66 (m, 3H), 1.38-1.48 (m, 1H), 1.18 (t, J=7.2 Hz, 1H), 0.94 (s, 3H).

### Synthesis of (R)-2-((1H-pyrrolo[2,3-b]pyridin-5-yl)oxy)-4-(4-((4-((4-(tert-butoxycarbonyl)piperazin-1-yl)methyl)-4′-chloro-4-methyl-3,4,5,6-tetrahydro-[1,1′-biphenyl]-2-yl)methyl)piperazin-1-yl)benzoic Acid (Intermediate NU)

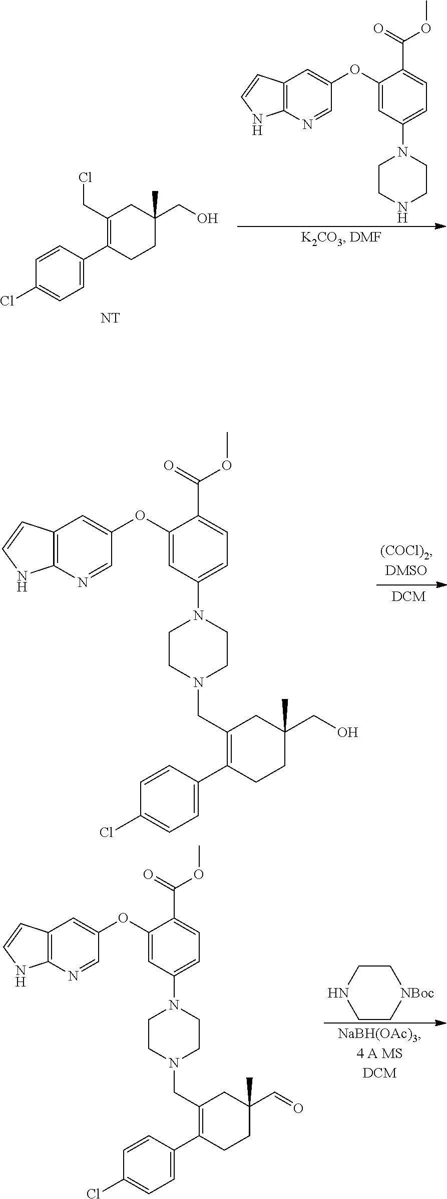

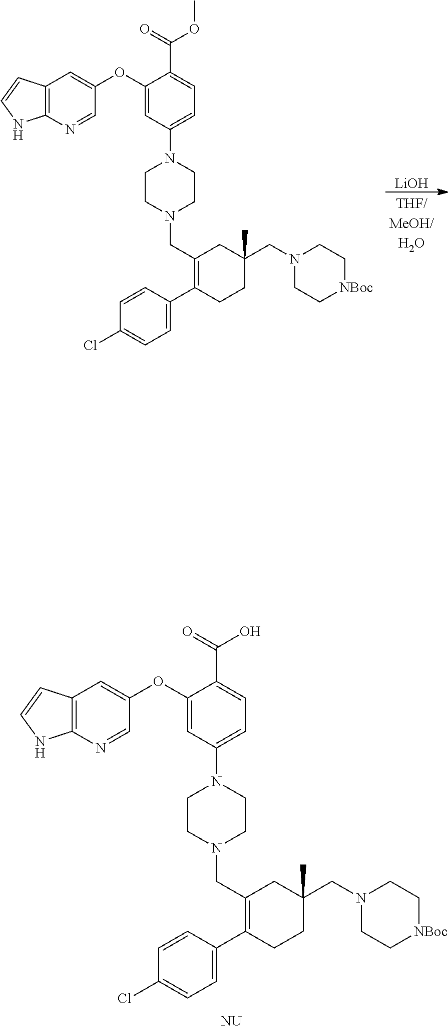

### Step 1—(R)-methyl 2-((1H-pyrrolo[2,3-b]pyridin-5-yl)oxy)-4-(4-((4′-chloro-4-(hydroxymethyl)-4-methyl-3,4,5,6-tetrahydro-[1,1′-biphenyl]-2-yl)methyl)piperazin-1-yl)benzoate

To a solution of methyl 2-((1H-pyrrolo[2,3-b]pyridin-5-yl)oxy)-4-(piperazin-1-yl)benzoate (1.8 g, 5.11 mmol, synthesized via Step 1 of Intermediate NQ) in DMF (20 mL) was added K₂CO₃ (1.76 g, 12.8 mmol) and (R)-(4′-chloro-6-(chloromethyl)-4-methyl-2,3,4,5-tetrahydro-[1,1′-biphenyl]-4-yl) methanol (2.19 g, 7.66 mmol, Intermediate NT). The mixture was then stirred at 25° C. for 12 hrs. On completion, the mixture was diluted with H₂O (100 mL), and extracted with EtOAc (50 mL×3. The combined organic layer was washed with brine (40 mL×3), dried over anhydrous Na₂SO₄, filtered and concentrated to give a residue. The residue was purified by column chromatography (SiO₂, Petroleum ether/Ethyl acetate=10/1 to 1/1) to give the title compound (2.2 g, 70% yield) as a white solid. LC-MS (ESI⁺) m/z 601.4 (M+H)⁺.

### Step 2—(R)-methyl 2-((1H-pyrrolo[2,3-b]pyridin-5-yl)oxy)-4-(4-((4′-chloro-4-formyl-4-methyl-3,4,5,6-tetrahydro-[1,1′-biphenyl]-2-yl)methyl)piperazin-1-yl)benzoate

To a solution of DMSO (572 mg, 7.32 mmol) in DCM (50 mL) was added dropwise oxalyl dichloride (929 mg, 7.32 mmol) at −70° C. for 0.5 hr. After addition, the mixture was stirred at this temperature for 0.4 h, and then (R)-methyl 2-((1H-pyrrolo[2,3-b]pyridin-5-yl)oxy)-4-(4-((4′-chloro-4-(hydroxymethyl)-4-methyl-3,4,5,6-tetrahydro-[1,1′-biphenyl]-2-yl)methyl)piperazin-1-yl)benzoate (2.2 g. 3.66 mmol) in DCM (10 mL) was added dropwise at −70° C. The resulting mixture was stirred at −70° C. for 1 hour and then TEA (1.85 g, 18.3 mmol) was added dropwise at 0° C. The resulting mixture was then stirred at 0° C. for 0.1 hr. On completion, the mixture was diluted with H₂O (50 mL), and extracted with DCM (50 mL×3). The combined organic layer was dried over anhydrous Na₂SO₄, filtered and concentrated to give the title compound (2.2 g) as a green solid. LC-MS (ESI⁺) m/z 599.5 (M+H)⁺.

### Step 3—(R)-tert-butyl 4-((6-((4-(3-((1H-pyrrolo[2,3-b]pyridin-5-yl)oxy)-4-(methoxycarbonyl)phenyl)piperazin-1-yl)methyl)-4′-chloro-4-methyl-2,3,4,5-tetrahydro-[1,1′-biphenyl]-4-yl)methyl)piperazine-1-carboxylate

To a solution of (R)-methyl 2-((1H-pyrrolo[2,3-b]pyridin-5-yl)oxy)-4-(4-((4′-chloro-4-formyl-4-methyl-3,4,5,6-tetrahydro-[1,1′-biphenyl]-2-yl)methyl)piperazin-1-yl)benzoate (2.2 g, 3.67 mmol) in DCM (30 mL) was added tert-butyl piperazine-1-carboxylate (820 mg, 4.41 mmol, CAS #57260-71-6) and 4 Å molecular sieves (3 g) and the mixture was stirred at 20° C. for 1 hr. Then NaBH(OAc)₃ (1.95 g, 9.18 mmol) was added and the mixture was stirred at 20° C. for 11 hrs. On completion, the mixture was filtered and the filtrate was concentrated to give the crude product. The product was purified by reversed-phase HPLC (0.1% NH₃·H₂O) to give the title compound (500 mg, 15% yield) as a white solid. LC-MS (ESI⁺) m/z 769.5 (M+H)⁺.

### Step 4—(R)-2-((1H-pyrrolo[2,3-b]pyridin-5-yl)oxy)-4-(4-((4-((4-(tert-butoxycarbonyl)piperazin-1-yl)methyl)-4′-chloro-4-methyl-3,4,5,6-tetrahydro-[1,1′-biphenyl]-2-yl)methyl)piperazin-1-yl)benzoic Acid

To a solution of (R)-tert-butyl 4-((6-((4-(3-((1H-pyrrolo[2,3-b]pyridin-5-yl)oxy)-4-(methoxycarbonyl)phenyl)piperazin-1-yl)methyl)-4′-chloro-4-methyl-2,3,4,5-tetrahydro-[1,1′-biphenyl]-4-yl)methyl)piperazine-1-carboxylate (460 mg, 597 μmol) in THF (8 mL). H₂O (4 mL) and MeOH (8 mL) was added LiOH·H₂O (125 mg, 2.99 mmol). The mixture was then stirred at 50° C. for 12 hrs. On completion, the mixture was under reduced pressure to remove THF and MeOH, then the pH was adjusted to 3-4 with 1N HCl solution, and extracted with DCM (20 ml×3). The combined organic layer was dried over Na₂SO₄, filtered and concentrated to give the title compound (450 mg) as a white solid. LC-MS (ESI⁺) m/z 755.3 (M+H)⁺.

### Synthesis of (R)-2-((1H-pyrrolo[2,3-b]pyridin-5-yl)oxy)-4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide (Intermediate NV)

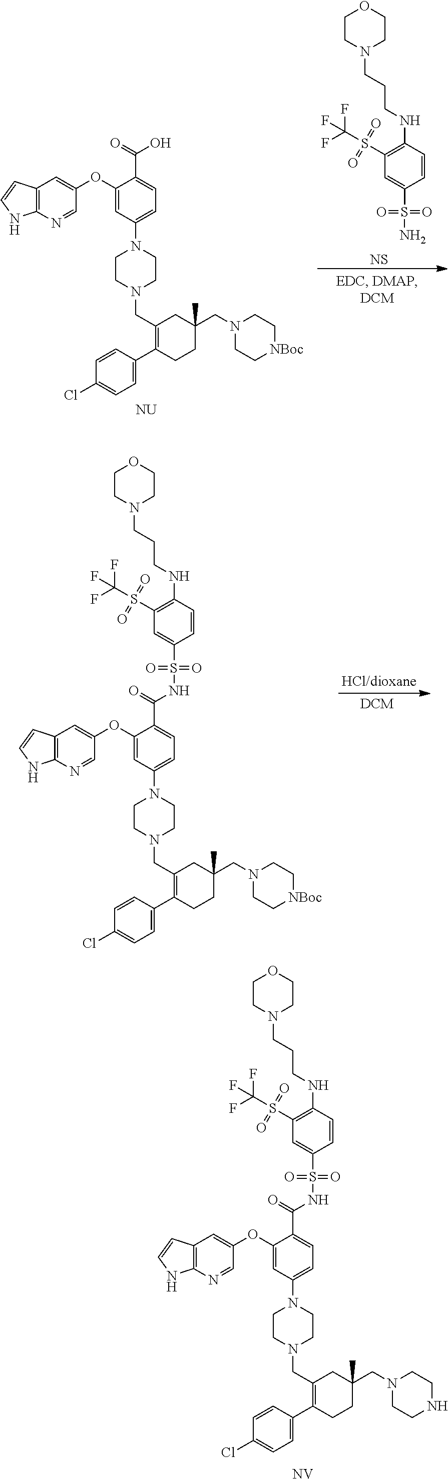

### Step 1—(R)-tert-butyl 4-((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate

To a solution of (R)-2-((1H-pyrrolo[2,3-b]pyridin-5-yl)oxy)-4-(4-((4-((4-(tert-butoxycarbonyl)piperazin-1-yl)methyl)-4′-chloro-4-methyl-3,4,5,6-tetrahydro-[1,1′-biphenyl]-2-yl)methyl)piperazin-1-yl)benzoic acid (200 mg, 265 μmol, Intermediate NU) in DCM (10 mL) was added EDC (144 mg, 927 μmol) and DMAP (146 mg, 1.19 mmol). Then 4-((3-morpholinopropyl)amino)-3-((trifluoromethyl)sulfonyl)benzenesulfonamide (137 mg, 318 μmol. Intermediate NS) was added and the mixture was stirred at 25° C. for 3 hrs. On completion, the mixture was diluted with H₂O (10 mL), and washed with 1M HCl (2 mL). The DCM phase was separated, dried over anhydrous Na₂SO₄, filtered and concentrated to give a residue. The residue was purified by column chromatography (SiO₂, Petroleum ether/Ethyl acetate=1/1 to DCM:MeOH=15:1) to give the title compound (260 mg, 81% yield) as a white solid. LC-MS (ESI⁺) m/z 1169.2 (M+H)⁺.

### Step 2—(R)-2-((1H-pyrrolo[2,3-b]pyridin-5-yl)oxy)-4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide

To a solution of (R)-tert-butyl 4-((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (260 mg, 222 μmol) in DCM (10 mL) was added HCl/dioxane (4 M, 2.5 mL). The mixture was then stirred at 20° C. for 1 hr. On completion, the mixture was concentrated to give the title compound (240 mg, HCl) as a white solid. LC-MS (ESI⁺) m/z 1068.5 (M+H)⁺.

### Synthesis of (R)-2-((1H-pyrrolo[2,3-b]pyridin-5-yl)oxy)-4-(4-((4-((4-(tert-butoxycarbonyl)piperazin-1-yl)methyl)-4′-chloro-[1,1′-biphenyl]-2-yl)(hydroxy)methyl)piperidin-1-yl)benzoic Acid (Intermediate NW)

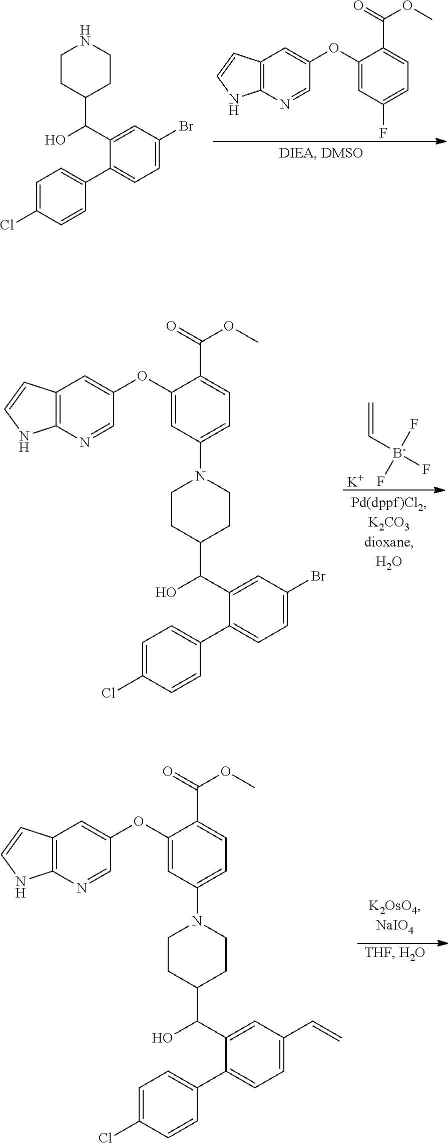

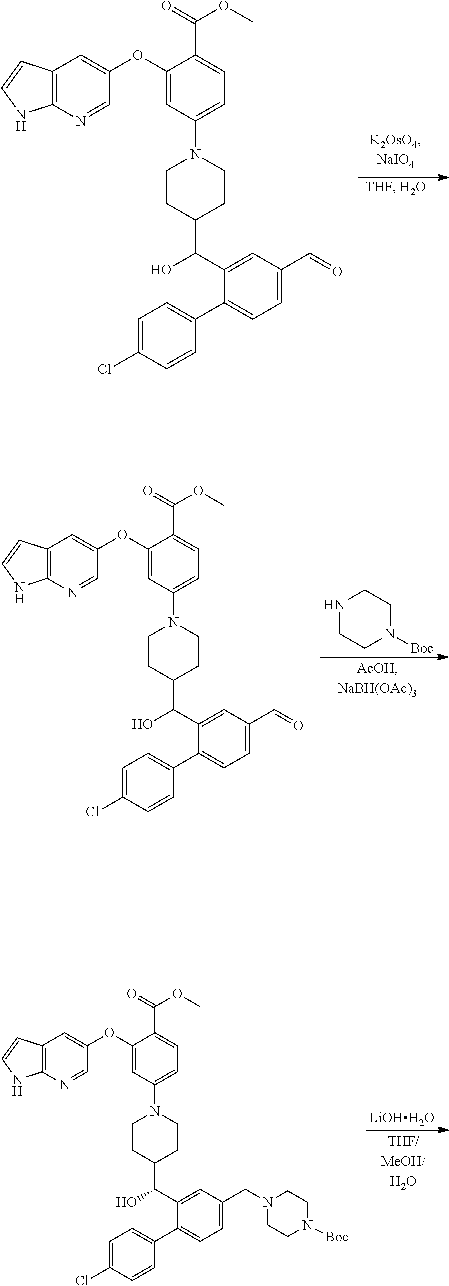 [SMILES: COC(=O)c1ccc(N2CCC(C(O)c3cc(C=O)ccc3-c3ccc(Cl)cc3)CC2)cc1Oc1cnc2[nH]ccc2c1 ; COC(=O)c1ccc(N2CCC(C(O)c3cc(C=O)ccc3-c3ccc(Cl)cc3)CC2)cc1Oc1cnc2[nH]ccc2c1 ; CC(C)(C)OC(=O)N1CCNCC1 ; COC(=O)c1ccc(N2CCC([C@@H](O)c3cc(CN4CCN(C(=O)OC(C)(C)C)CC4)ccc3-c3ccc(Cl)cc3)CC2)cc1Oc1cnc2[nH]ccc2c1 ; "LiOH·H2O"]

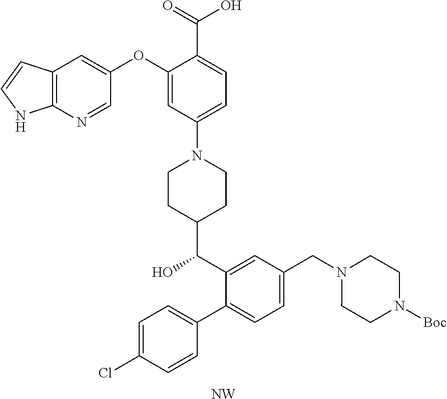

### Step 1—Methyl 2-((1H-pyrrolo[2,3-b]pyridin-5 chloro-[1,1′-biphenyl]-2-yl)(hydroxy)methylpiperidin-1-yl)benzoate

To a solution of [5-bromo-2-(4-chlorophenyl)phenyl]-(4-piperidyl) methanol (4 g, 9.59 mmol, HCl, synthesized via Steps 1-3 of Intermediate KU) in DMSO (50 mL) was added DIEA (12.4 g, 95.9 mmol) and methyl 4-fluoro-2-(1H-pyrrolo[2,3-b]pyridin-5-yloxy)benzoate (2.74 g, 9.59 mmol, CAS #1235865-75-4). The mixture was then stirred at 120° C. for 12 hrs. On completion, the reaction mixture was quenched with H₂O (100 mL) and extracted with EtOAc (100 mL×3). The combined organic layers were washed with brine (50 mL×2), dried over Na₂SO₄ filtered and the filtrate was concentrated under reduced pressure to give a residue. The residue was purified by column chromatography (SiO₂, Petroleum ether/Ethyl acetate=10/1 to 5/1) to give the title compound (2.7 g, 42% yield) as a yellow solid. LC-MS (ESI+) m/z 647.9 (M+H)⁺.

### Step 2—Methyl 2-((1H-pyrrolo[2,3-b]pyridin-5-yl)oxy)-4-(4-((4′-chloro-4-vinyl-[1,1′-biphenyl]-2-yl)(hydroxy)methyl)piperidin-1-yl)benzoate

To a solution of methyl 4-[4-[[5-bromo-2-(4-chlorophenyl)phenyl]-hydroxy-methyl]-1-piperidyl]-2-(1H-pyrrolo[2,3-b]pyridin-5-yloxy)benzoate (2.7 g, 4.17 mmol), potassium trifluoro (vinyl) boranuide (838 mg, 6.26 mmol, CAS #13682-77-4), Pd(dppf)Cl₂ (305 mg, 417 μmol), and K₂CO₃ (1.73 g, 12.5 mmol) in dioxane (12 mL) and H₂O (3 mL) was degassed and purged with N₂ three times. Then the mixture was stirred at 80° C. for 2 hrs under N₂ atmosphere. On completion, the reaction mixture was concentrated under reduced pressure to give a residue. The residue was purified by column chromatography (SiO₂, Petroleum ether/Ethyl acetate=1/0 to 1/1) to give the title compound (2.2 g, 83% yield) as a black solid. LC-MS (ESI+) m/z 594.2 (M+H)⁺.

### Step 3—Methyl 2-((1H-pyrrolo[2,3-b]pyridin-5-yl)oxy)-4-(4-((4′-chloro-4-formyl-[1,1′-biphenyl]-2-yl)(hydroxy)methyl)piperidin-1-yl)benzoate

To a solution of methyl 4-[4-[[2-(4-chlorophenyl)-5-vinyl-phenyl]-hydroxy-methyl]-1-piperidyl]-2-(1H-pyrrolo[2,3-b]pyridin-5-yloxy)benzoate (2.2 g, 3.70 mmol), K₂OsO₄·2H₂O (95 mg, 259 μmol, CAS #10022-66-9), and NaIO₄ (3.96 g, 18.5 mmol, CAS #7790-28-5) in THF (15 mL) and H₂O (3 mL) was degassed and purged with N₂ three times. Then the mixture was stirred at 25° C. for 1 hr under N₂ atmosphere. On completion, the reaction mixture was concentrated under reduced pressure to give a residue. The residue was purified by prep-HPLC (neutral condition) to give the title compound (700 mg, 32% yield) as a white solid. LC-MS (ESI+) m/z 596.2 (M+H)⁺.

### Step 4—Tert-butyl (R)-4-((2-((1-(3-((1H-pyrrolo[2,3-b]pyridin-5-yl)oxy)-4-(methoxycarbonyl)phenyl)piperidin-4-yl)(hydroxy)methyl)-4′-chloro-[1,1′-biphenyl]-4-yl)methyl)piperazine-1-carboxylate

To a solution of tert-butyl piperazine-1-carboxylate (248 mg, 1.33 mmol) in DMSO (10 mL) was added KOAc (163 mg, 1.67 mmol), AcOH (199 mg, 3.33 mmol, 190 uL) and methyl 4-[4-[[2-(4-chlorophenyl)-5-formyl-phenyl]-hydroxy-methyl]-1-piperidyl]-2-(1H-pyrrolo[2,3-b]pyridin-5-yloxy)benzoate (660 mg, 1.11 mmol) at 25° C. for 1 hr. Then NaBH(OAc)₃ (588 mg, 2.78 mmol) was added at 0° C. and the mixture was stirred at 25° C. for 1 hr. On completion, the reaction mixture was concentrated under reduced pressure to give a residue. The residue was purified by column chromatography (SiO₂, Petroleum ether/Ethyl acetate=1/0 to 0/1) to give the title compound (600 mg, 63% yield) as a yellow oil. LC-MS (ESI+) m/z 766.6 (M+H)⁺.

### Step 5—(R)-2-((1H-pyrrolo[2,3-b]pyridin-5-yl)oxy)-4-(4-((4-((4-(tert-butoxycarbonyl)piperazin-1-yl)methyl)-4′-chloro-[1,1′-biphenyl]-2-yl)(hydroxy)methyl)piperidin-1-yl)benzoic Acid

To a solution of tert-butyl 4-[[4-(4-chlorophenyl)-3-[(R)-hydroxy-[1-[4-methoxycarbonyl-3-(1H-pyrrolo[2,3-b]pyridin-5-yloxy)phenyl]-4-piperidyl]methyl]phenyl]methyl]piperazine-1-carboxylate (200 mg, 260 μmol) in THF (2 mL), MeOH (0.5 mL) and H₂O (0.5 mL) was added LiOH·H₂O (32 mg, 782 μmol). The mixture was then stirred at 40° C. for 12 hrs. On completion, the reaction mixture was concentrated under reduced pressure to give a residue. The residue was purified by prep-HPLC (neutral condition) to give the title compound (120 mg, 57% yield) as a white solid. LC-MS (ESI⁺) m/z 752.3 (M+H)⁺.

### Synthesis of 4-((3-(Dimethylamino)propyl)amino)-3-((trifluoromethyl)sulfonyl)benzenesulfonamide (Intermediate NX)

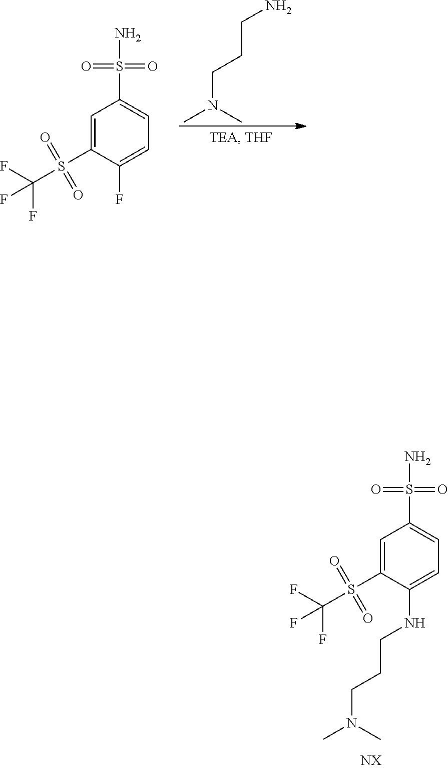

A mixture of 4-fluoro-3-((trifluoromethyl)sulfonyl)benzenesulfonamide (500 mg, 1.63 mmol, CAS #1027345-08-9), N,N-dimethylpropane-1,3-diamine (199 mg, 1.95 mmol, 244 μL, CAS #109-55-7), and TEA (494 mg, 4.88 mmol, 680 uL) in THF (5 mL) was degassed and purged with N₂ three times. Then the mixture was stirred at 50° C. for 12 hrs under N₂ atmosphere. On completion, the reaction mixture was concentrated under reduced pressure to give a residue. The residue was purified by column chromatography (SiO₂, DCM:MeOH=20/1 to 10/1) to give the title compound (504 mg, 79% yield) as a yellow solid. LC-MS (ESI⁺) m/z 390.0 (M+H)⁺.

### Synthesis of (R)-2-((1H-pyrrolo[2,3-b]pyridin-5-yl)oxy)-4-(4-((4′-chloro-4-(piperazin-1-ylmethyl)-[1,1′-biphenyl]-2-yl)(hydroxy)methyl)piperidin-1-yl)-N-((4-((3-(dimethylamino)propyl)amino)-3-((trifluoromethyl)sulfonyl)phenyl)sulfonyl)benzamide (Intermediate NY)

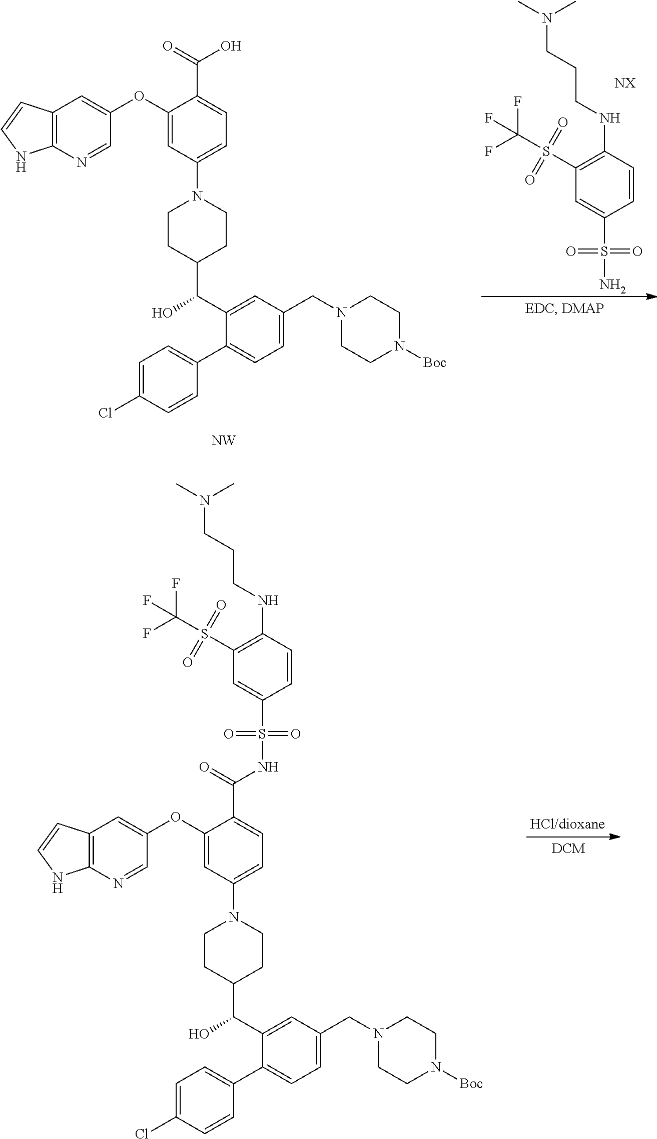

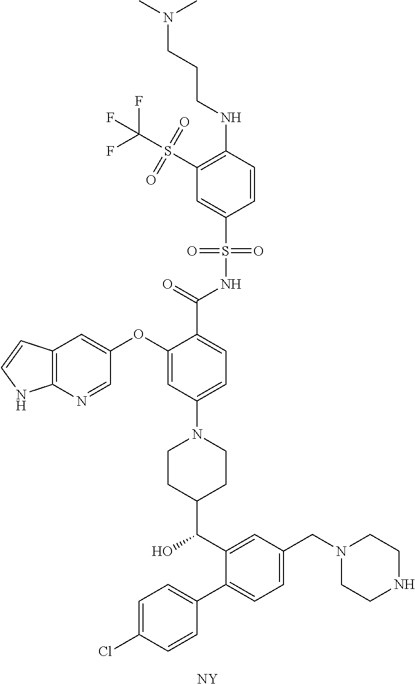

### Step 1—tert-butyl (R)-4-((2-((1-(3-((1H-pyrrolo[2,3-b]pyridin-5-yl)oxy)-4-(((4-((3-(dimethylamino)propyl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)methyl)piperazine-1-carboxylate

To a solution of 4-[4-[(R)-[5-[(4-tert-butoxycarbonylpiperazin-1-yl)methyl]-2-(4-chlorophenyl)phenyl]-hydroxy-methyl]-1-piperidyl]-2-(1H-pyrrolo[2,3-b]pyridin-5-yloxy)benzoic acid (70 mg, 93 μmol, Intermediate NW) in DCM (1 mL) was added EDC (36.1 mg, 232 μmol, 41.1 uL), DMAP (28 mg, 232 μmol) and 4-[3-(dimethylamino)propylamino]-3-(trifluoromethylsulfonyl)benzenesulfonamide (36 mg, 93 μmol, Intermediate NX). The mixture was then stirred at 25° C. for 2 hrs. On completion, the reaction mixture was concentrated under reduced pressure to give a residue. The residue was purified by prep-HPLC (neutral condition) to give the title compound (100 mg, 87% yield) as a white solid. LC-MS (ESI+) m/z 1123.3 (M+H)⁺.

### Step 2—(R)-2-((1H-pyrrolo[2,3-b]pyridin-5-yl)oxy)-4-(4-((4′-chloro-4-(piperazin-1-ylmethyl)-[1,1′-biphenyl]-2-yl)(hydroxy)methyl)piperidin-1-yl)-N-((4-((3-(dimethylamino)propyl)amino)-3-((trifluoromethyl)sulfonyl)phenyl)sulfonyl)benzamide

To a solution of tert-butyl 4-[[4-(4-chlorophenyl)-3-[(R)-[1-[4-[[4-[3-(dimethylamino)propylamino]-3-(trifluoromethylsulfonyl)phenyl]sulfonylcarbamoyl]-3-(1H-pyrrolo[2,3-b]pyridin-5-yloxy)phenyl]-4-piperidyl]-hydroxy-methyl]phenyl]methyl]piperazine-1-carboxylate (75 mg, 66 μmol) in DCM (1 mL) was added HCl/dioxane (4 M, 16.6 uL). The mixture was then stirred at 25° C. for 1.5 hrs. On completion, the reaction mixture was concentrated under reduced pressure to give a title compound (65 mg, HCl) as a white solid. LC-MS (ESI+) m/z 1023.0 (M+H)⁺.

### Synthesis of Tert-butyl methyl (3-((4-sulfamoyl-2-((trifluoromethyl)sulfonyl)phenyl)amino)propyl)carbamate (Intermediate NZ)

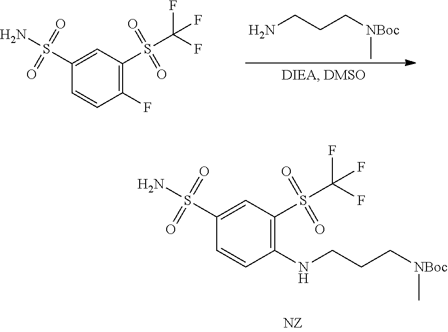

To a solution of 4-fluoro-3-((trifluoromethyl)sulfonyl)benzenesulfonamide (2 g, 6.51 mmol, CAS #1027345-08-9) and tert-butyl (3-aminopropyl)(methyl)carbamate (1.23 g, 6.51 mmol, CAS #150349-36-3) in THF (20 mL) was added TEA (1.98 g, 19.5 mmol). The mixture was stirred at 50° C. for 12 hrs. On completion, the reaction mixture was diluted by water (60 mL) and extracted by ethyl acetate (3×50 mL). The combined organic layers were washed by brine (150 mL) and dried over anhydrous sodium sulfate, filtered and the filtrate was concentrated in vacuo to give the title compound (3 g) as a white solid. LC-MS (ESI⁺) m/z 376.3 (M+H)⁺.

### Synthesis of (S)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3-(methylamino)propyl)amino)-3-((trifluoromethyl)sulfonyl)phenyl)sulfonyl)benzamide (Intermediate OA)

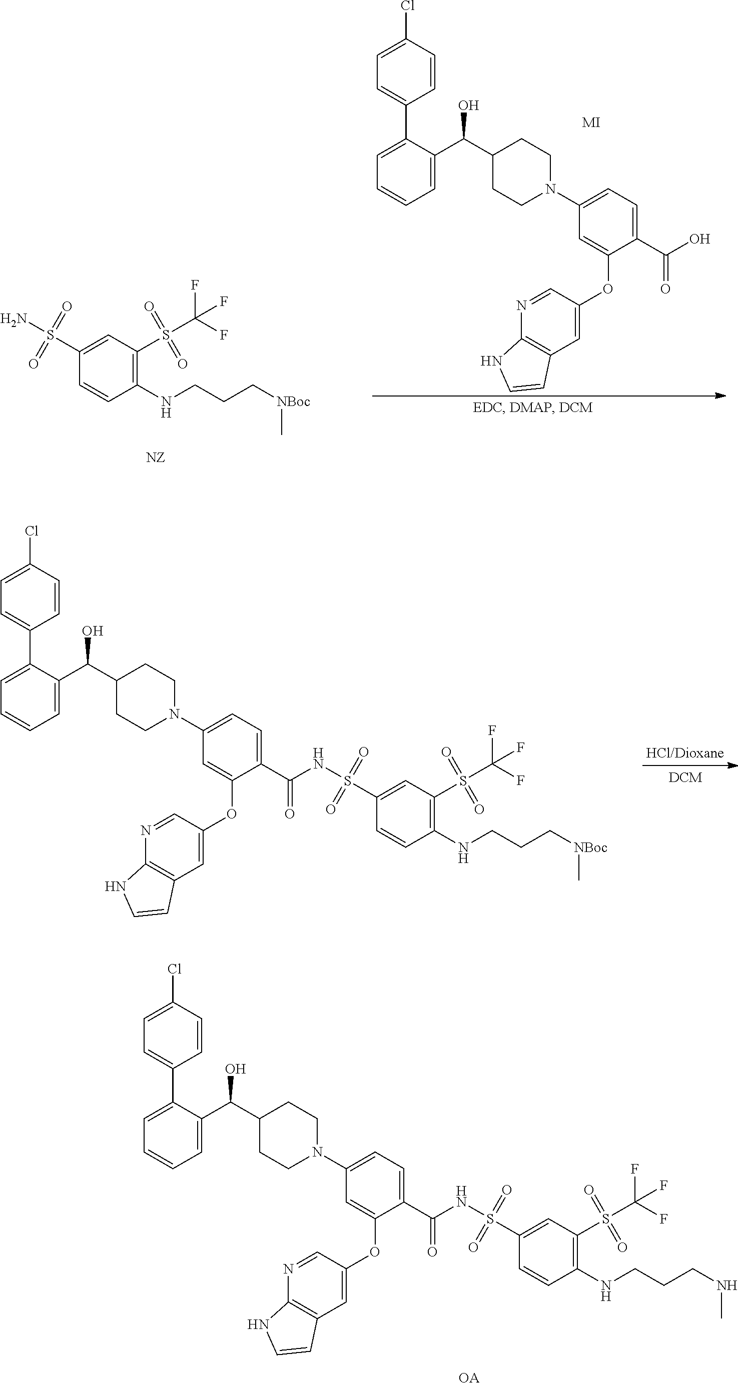

### Step 1—(S)-tert-butyl (3-((4-(N-(2-((1H-pyrrolo[2,3-b]pyridin-5-yl)oxy)-4-(4-((4′-chloro-[1,1′-biphenyl]-2-yl)(hydroxy)methyl)piperidin-1-yl)benzoyl)sulfamoyl)-2-((trifluoromethyl)sulfonyl)phenyl)amino)propyl)(methyl)carbamate

To a solution of (S)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoic acid (300 mg, 541 μmol, Intermediate MI) in DCM (3 mL) was added tert-butyl methyl (3-((4-sulfamoyl-2-((trifluoromethyl)sulfonyl)phenyl)amino)propyl)carbamate (309 mg, 650 μmol, Intermediate NZ), EDC (210 mg, 1.35 mmol) and DMAP (165 mg, 1.35 mmol). The mixture was then stirred at 25° C. for 4 hrs. On completion, the reaction mixture was concentrated under reduced pressure to give a crude product. The crude product was purified by reversed-phase HPLC (0.1% FA condition) to give the title compound (350 mg, 50% yield) as a gray solid. LC-MS (ESI⁺) m/z 1013.0 (M+H)⁺.

### Step 2—(S)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3-(methylamino)propyl)amino)-3-((trifluoromethyl)sulfonyl)phenyl)sulfonyl)benzamide

To a solution of (S)-tert-butyl (3-((4-(N-(2-((1H-pyrrolo[2,3-b]pyridin-5-yl)oxy)-4-(4-((4′-chloro-[1,1′-biphenyl]-2-yl)(hydroxy)methyl)piperidin-1-yl)benzoyl)sulfamoyl)-2-((trifluoromethyl)sulfonyl)phenyl)amino)propyl)(methyl)carbamate (280 mg, 277 μmol) in DCM (5 mL) was added HCl/dioxane (4 M, 1 mL). The mixture was then stirred at 25° C. for 2 hrs. On completion, the reaction mixture was concentrated under reduced pressure to give the crude product. The crude product was extracted by DCM (20 mL) and concentrated in vacuo to give the title compound (320 mg. HCl) as a white solid. LC-MS (ESI⁺) m/z 912.2 (M+H)⁺.

### Synthesis of (2S,4R)-1-((S)-3,3-dimethyl-2-(6-oxohexanamido)butanoyl)-4-hydroxy-N—((S)-1-(4-(4-methylthiazol-5-yl)phenyl)ethyl)pyrrolidine-2-carboxamide (Intermediate OB)

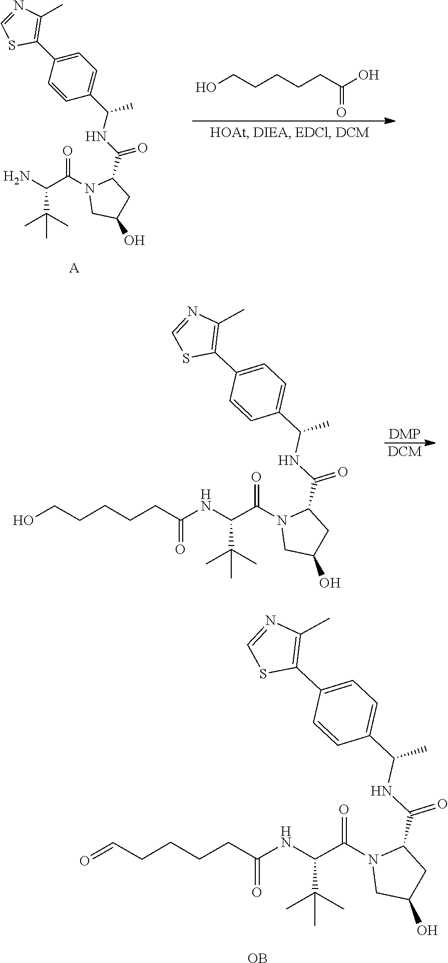

### Step 1—(2S,4R)-4-hydroxy-1-((S)-2-(6-hydroxyhexanamido)-3,3-dimethylbutanoyl)-N—((S)-1-(4-(4-methylthiazol-5-yl)phenyl)ethyl)pyrrolidine-2-carboxamide

To a solution of 6-hydroxyhexanoic acid (618 mg, 4.68 mmol, CAS #1191-25-9), in DCM (5 mL) was added EDCI (1.49 g, 7.80 mmol), DIEA (4.03 g, 31.2 mmol), (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (1.5 g, 3.12 mmol, HCl, Intermediate A) and HOAt (1.06 g, 7.80 mmol, 1.09 mL). The mixture was stirred at 25° C. for 1 hr. On completion, the reaction mixture was concentrated under reduced pressure to remove solvent to give the crude product. The crude product was purified by reversed-phase HPLC (0.1% FA condition) to give the title compound (900 mg, 51% yield) as a white solid. LC-MS (ESI⁺) m/z 559.4 (M+H)⁺.

### Step 2—(2S,4R)-1-((S)-3,3-dimethyl-2-(6-oxohexanamido)butanoyl)-4-hydroxy-N—((S)-1-(4-(4-methylthiazol-5-yl)phenyl)ethyl)pyrrolidine-2-carboxamide

To a solution of (2S,4R)-4-hydroxy-1-((S)-2-(6-hydroxyhexanamido)-3,3-dimethylbutanoyl)-N—((S)-1-(4-(4-methylthiazol-5-yl)phenyl)ethyl)pyrrolidine-2-carboxamide (250 mg, 447 μmol) in DCM (2 mL) was added DMP (285 mg, 671 μmol). The mixture was then stirred at 25° C. for 0.5 hrs. On completion, the reaction mixture was filtered to give a filtrate. The filtrate was washed with NaHCO₃ (30 mL), dried over anhydrous sodium sulfate, filtered and the filtrate was concentrated under reduced pressure to give the title compound (200 mg) as a white solid. LC-MS (ESI⁺) m/z 557.5 (M+H)⁺.

### Synthesis of Tert-butyl (3-((4-sulfamoyl-2-((trifluoromethyl)sulfonyl)phenyl)amino)propyl) carbamate (Intermediate OC)

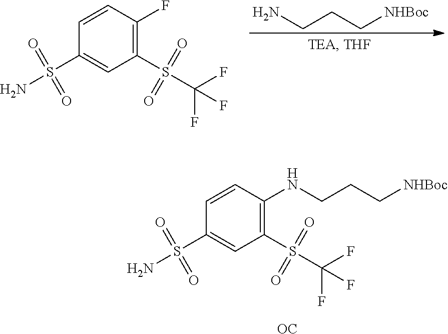 [SMILES: NS(=O)(=O)c1ccc(F)c(S(=O)(=O)C(F)(F)F)c1 ; CC(C)(C)OC(=O)NCCCN ; CC(C)(C)OC(=O)NCCCNc1ccc(S(N)(=O)=O)cc1S(=O)(=O)C(F)(F)F ; "OC"]

To a solution of 4-fluoro-3-((trifluoromethyl)sulfonyl)benzenesulfonamide (2.0 g, 6.5 mmol, CAS #1027345-08-9) in THF (20 mL) was added TEA (1.98 g, 19.5 mmol, 2.72 mL) and tert-butyl (3-aminopropyl)carbamate (1.25 g, 7.16 mmol, 1.25 mL, CAS #75178-96-0). The mixture was then stirred at 50° C. for 12 hrs. On completion, the reaction mixture was quenched with H₂O (20 mL) at 25° C., and then extracted by ethyl acetate (80 mL×3). The combined organic layers were washed by brine (200 mL) and dried over anhydrous sodium sulfate, filtered and the filtrate was concentrated in vacuo to give the title compound (2.5 g) as a white solid. LC-MS (ESI⁺) m/z 406.1. (M+H)⁺.

### Synthesis of (R)-2-((1H-pyrrolo[2,3-b]pyridin-5-yl)oxy)-N-((4-((3-aminopropyl)amino)-3-((trifluoromethyl)sulfonyl)phenyl)sulfonyl)-4-(4-((4′-chloro-[1,1′-biphenyl]-2-yl)(hydroxy)methyl)piperidin-1-yl)benzamide (Intermediate OD)

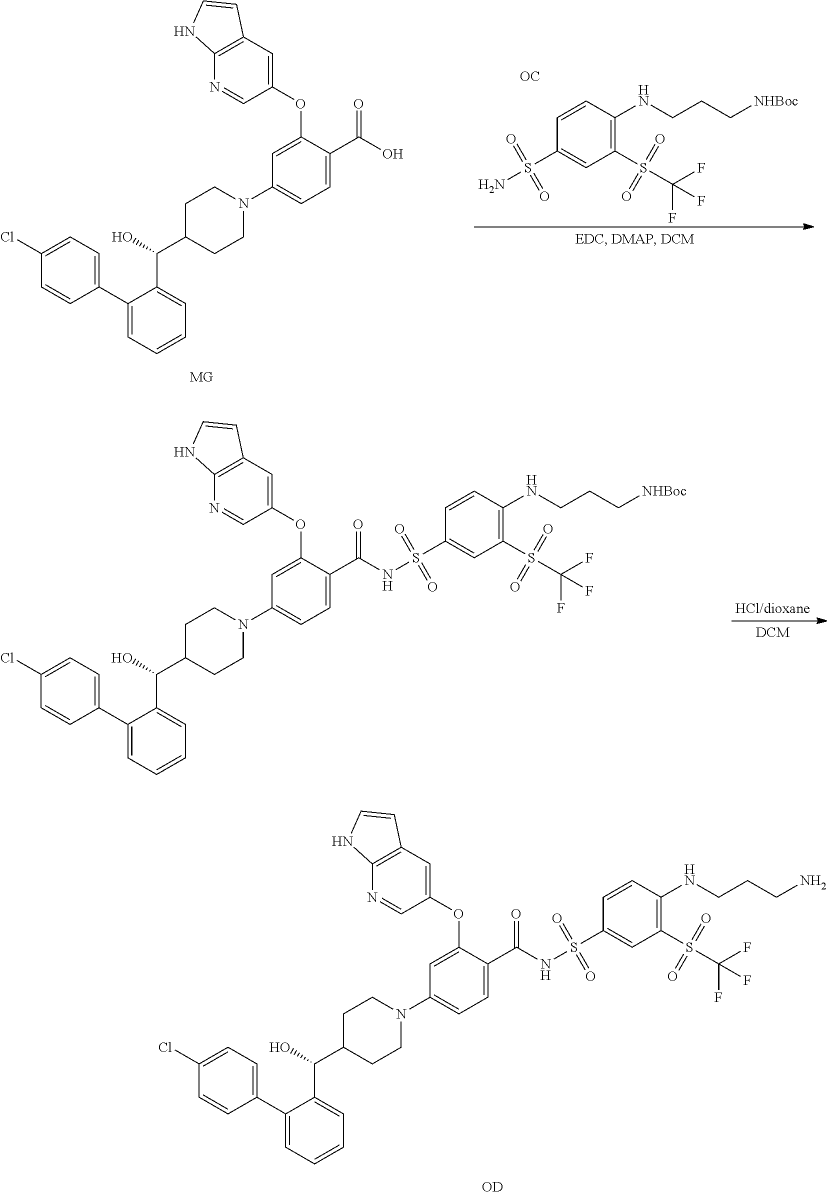 [SMILES: O=C(O)c1ccc(N2CCC([C@@H](O)c3ccccc3-c3ccc(Cl)cc3)CC2)cc1Oc1cnc2[nH]ccc2c1 ; CC(C)(C)OC(=O)NCCCNc1ccc(S(N)(=O)=O)cc1S(=O)(=O)C(F)(F)F ; "OC" ; CC(C)(C)OC(=O)NCCCNc1ccc(S(=O)(=O)NC(=O)c2ccc(N3CCC([C@@H](O)c4ccccc4-c4ccc(Cl)cc4)CC3)cc2Oc2cnc3[nH]ccc3c2)cc1S(=O)(=O)C(F)(F)F ; NCCCNc1ccc(S(=O)(=O)NC(=O)c2ccc(N3CCC([C@@H](O)c4ccccc4-c4ccc(Cl)cc4)CC3)cc2Oc2cnc3[nH]ccc3c2)cc1S(=O)(=O)C(F)(F)F ; "OD"]

### Step 1—(R)-tert-butyl (3-((4-(N-(2-((1H-pyrrolo[2,3-b]pyridin-5-yl)oxy)-4-(4-((4′-chloro-[1,1′-biphenyl]-2-yl)(hydroxy)methyl)piperidin-1-yl)benzoyl)sulfamoyl)-2-((trifluoromethyl)sulfonyl)phenyl)amino)propyl)carbamate

To a solution of (R)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoic acid (200 mg, 361 μmol, Intermediate MG) in DCM (4 mL) was added DMAP (110 mg, 902 μmol), EDC (112 mg, 722 μmol, 128 uL) and tert-butyl (3-((4-sulfamoyl-2-((trifluoromethyl)sulfonyl)phenyl)amino)propyl)carbamate (200 mg, 433 μmol, Intermediate OC). The mixture was then stirred at 25° C. for 2 hrs. On completion, the reaction mixture was concentrated under reduced pressure to give a residue. The residue was purified by reversed-phase HPLC (0.8 g/L ammonium bicarbonate condition) to give the title compound (130 mg, 36% yield) as a white solid. LC-MS (ESI⁺) m/z 998.2. (M+H)⁺.

### Step 2—(R)-2-((1H-pyrrolo[2,3-b]pyridin-5-yl)oxy)-N-((4-((3-aminopropyl)amino)-3-((trifluoromethyl)sulfonyl)phenyl)sulfonyl)-4-(4-((4′-chloro-[1,1′-biphenyl]-2-yl)(hydroxy)methyl)piperidin-1-yl)benzamide

To a solution of (R)-tert-butyl (3-((4-(N-(2-((1H-pyrrolo[2,3-b]pyridin-5-yl)oxy)-4-(4-((4′-chloro-[1,1′-biphenyl]-2-yl)(hydroxy)methyl)piperidin-1-yl)benzoyl)sulfamoyl)-2-((trifluoromethyl)sulfonyl)phenyl)amino)propyl)carbamate (130 mg, 130 μmol) in DCM (2 mL) was added HCl/dioxane (4 M, 0.5 mL). The reaction was then stirred at 25° C. for 1 hr. On completion, the reaction mixture was concentrated under reduced pressure to give the title compound (120 mg) as a white solid. LC-MS (ESI⁺) m/z 897.1. (M+H)⁺.

### Synthesis of (S)-2-((1H-pyrrolo[2,3-b]pyridin-5-yl)oxy)-N-((4-((3-aminopropyl)amino)-3-((trifluoromethyl)sulfonyl)phenyl)sulfonyl)-4-(4-((4′-chloro-[1,1′-biphenyl]-2-yl)(hydroxy)methyl)piperidin-1-yl)benzamide (Intermediate QE)

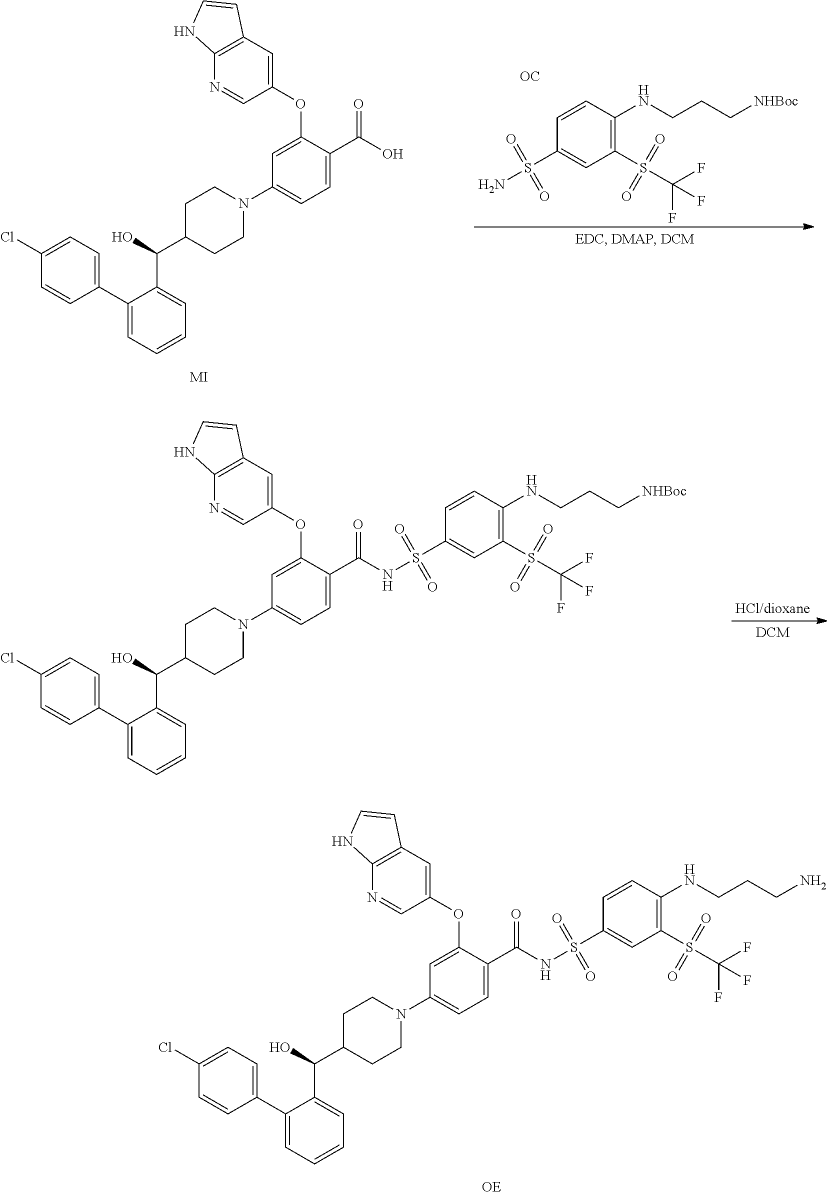

### Step 1—Tert-butyl (S)-(3-((4-(N-(2-((1H-pyrrolo[2,3-b]pyridin-5-yl)oxy)-4-(4-((4′-chloro-[1,1′-biphenyl]-2-yl)(hydroxy)methyl)piperidin-1-yl)benzoyl)sulfamoyl)-2-((trifluoromethyl)sulfonyl)phenyl)amino)propyl)carbamate

To a solution of (S)-2-((1H-pyrrolo[2,3-b]pyridin-5-yl)oxy)-4-(4-((4′-chloro-[1,1′-biphenyl]-2-yl)(hydroxy)methyl)piperidin-1-yl)benzoic acid (200 mg, 361 μmol, Intermediate MI) in DCM (4 mL) was added DMAP (110 mg, 902 μmol), EDC (112 mg, 722 μmol, 128 uL) and tert-butyl (3-((4-sulfamoyl-2-((trifluoromethyl)sulfonyl)phenyl)amino)propyl)carbamate (200 mg, 433 μmol, Intermediate OC). The mixture was stirred at 25° C. for 12 hrs. On completion, the reaction mixture was concentrated under reduced pressure to give a residue. The residue was purified by reversed-phase HPLC (0.1% FA condition) to give the title compound (200 mg, 50% yield) as a white solid. LC-MS (ESI⁺) m/z 997.1. (M+H)⁺.

### Step 2—(S)-2-((1H-pyrrolo[2,3-b]pyridin-5-yl)oxy)-N-((4-((3-aminopropyl)amino)-3-((trifluoromethyl)sulfonyl)phenyl)sulfonyl)-4-(4-((4′-chloro-[1,1′-biphenyl]-2-yl)(hydroxy)methyl)piperidin-1-yl)benzamide

To a solution of tert-butyl (S)-(3-((4-(N-(2-((1H-pyrrolo[2,3-b]pyridin-5-yl)oxy)-4-(4-((4′-chloro-[1,1′-biphenyl]-2-yl)(hydroxy)methyl)piperidin-1-yl)benzoyl)sulfamoyl)-2-((trifluoromethyl)sulfonyl)phenyl)amino)propyl)carbamate (200 mg, 180 μmol) in DCM (2 mL) was added HCl/dioxane (4 M, 0.14 mL). The reaction was stirred at 25° C. for 2 hrs. On completion, the reaction mixture was concentrated under reduced pressure to give the title compound (200 mg) as a white solid. LC-MS (ESI⁺) m/z 896.9. (M+H)⁺.

### Synthesis of 5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic Acid (Intermediate OF)

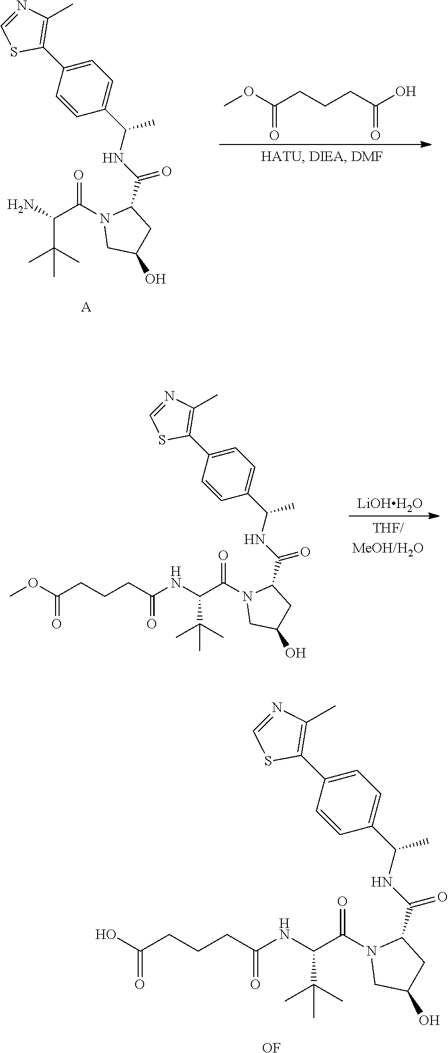

### Step 1—Methyl 5-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoate

To a solution of 5-methoxy-5-oxo-pentanoic acid (328 mg, 2.25 mmol, CAS #1501-27-5) in DMF (10 mL) was added HATU (1.03 g, 2.70 mmol), DIEA (1.45 g, 11.2 mmol, 1.96 mL) and (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (1 g, 2.25 mmol, Intermediate A). The mixture was then stirred at 25° C. for 1 hr. On completion, the reaction mixture was diluted with H₂O (15 mL) and extracted with EA (30 mL×3). The combined organic layers dried over Na₂SO₄, filtered and concentrated under reduced pressure to give the title compound (1 g) as a yellow solid. LC-MS (ESI⁺) m/z 573.4 (M+H)⁺.

### Step 2—5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic Acid

To a solution of methyl 5-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-5-oxo-pentanoate (1 g, 1.75 mmol) in THF (8 mL), MeOH (2 mL) and H₂O (2 mL) was added LiOH·H₂O (293 mg, 6.98 mmol). The mixture was then stirred at 40° C. for 12 hrs. On completion, the reaction mixture was concentrated under reduced pressure to give a residue, then HCl (1M) was added until the pH=7. The reaction mixture was diluted with H₂O (15 mL) and extracted with EA (30 mL×3). The combined organic layers dried over Na₂SO₄, filtered and concentrated under reduced pressure to give the title compound (970 mg) as a white solid. LC-MS (ESI+) m/z 559.5 (M+H)⁺.

### Synthesis of 2-((1H-pyrrolo[2,3-b]pyridin-5-yl)oxy)-4-(4-methylpiperazin-1-yl)benzoic Acid (Intermediate OG)

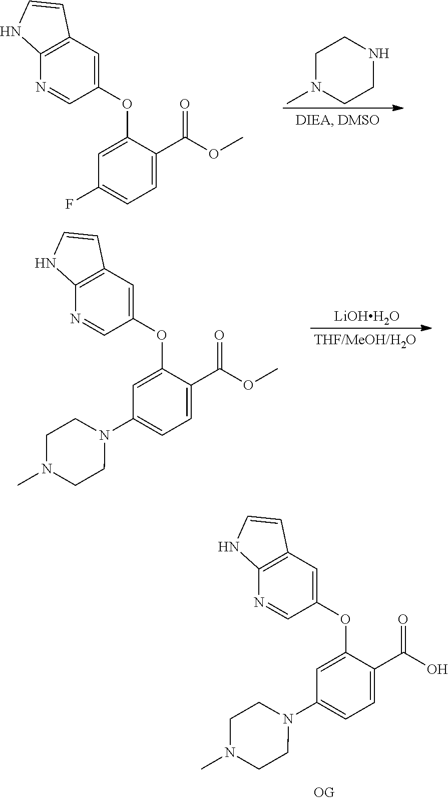

### Step 1—Methyl 2-((1H-pyrrolo[2,3-b]pyridin-5-yl)oxy)-4-(4-methylpiperazin-1-yl)benzoate

To a solution of 1-methylpiperazine (2 g, 19 mmol, 2.21 MI, CAS #109-01-3), methyl 4-fluoro-2-(7H-pyrrolo[2,3-b]pyridin-5-yloxy)benzoate (5.72 g, 19.9 mmol, CAS #1235865-75-4), and DIEA (12.9 g, 99 mmol, 17.3 mL) in DMSO (20 mL) was degassed and purged with N₂ three times. Then the mixture was stirred at 120° C. for 12 hrs under N₂ atmosphere. On completion, the crude product was triturated with H₂O at 25° C. for 15 min. Then the mixture was filtered and the filtrate cake was dried under reduced pressure to give the title compound (5 g) as a white solid. LC-MS (ESI+) m/z 367.3 (M+H)⁺.

### Step 2—2-((1H-pyrrolo[2,3-b]pyridin-5-yl)oxy)-4-(4-methylpiperazin-1-yl)benzoic Acid

To a solution of methyl 4-(4-methylpiperazin-1-yl)-2-(7H-pyrrolo[2,3-b]pyridin-5-yloxy)benzoate (2.00 g, 5.46 mmol) in THF (4 mL), MeOH (1 mL) and H₂O (1 mL) was added LiOH·H₂O (916 mg, 21.8 mmol). The mixture was then stirred at 40° C. for 12 hrs. On completion, the reaction mixture was concentrated under reduced pressure to give a residue. The residue was purified by prep-HPLC (neutral condition) to give the title compound (1.8 g, 91% yield) as a white solid. LC-MS (ESI+) m/z 353.2 (M+H)⁺.

### Synthesis of 2-((1H-pyrrolo[2,3-b]pyridin-5-yl)oxy)-4-(4-methylpiperazin-1-yl)-N-((4-((3-(piperazin-1-yl)propyl)amino)-3-((trifluoromethyl)sulfonyl)phenyl)sulfonyl)benzamide (Intermediate OH)

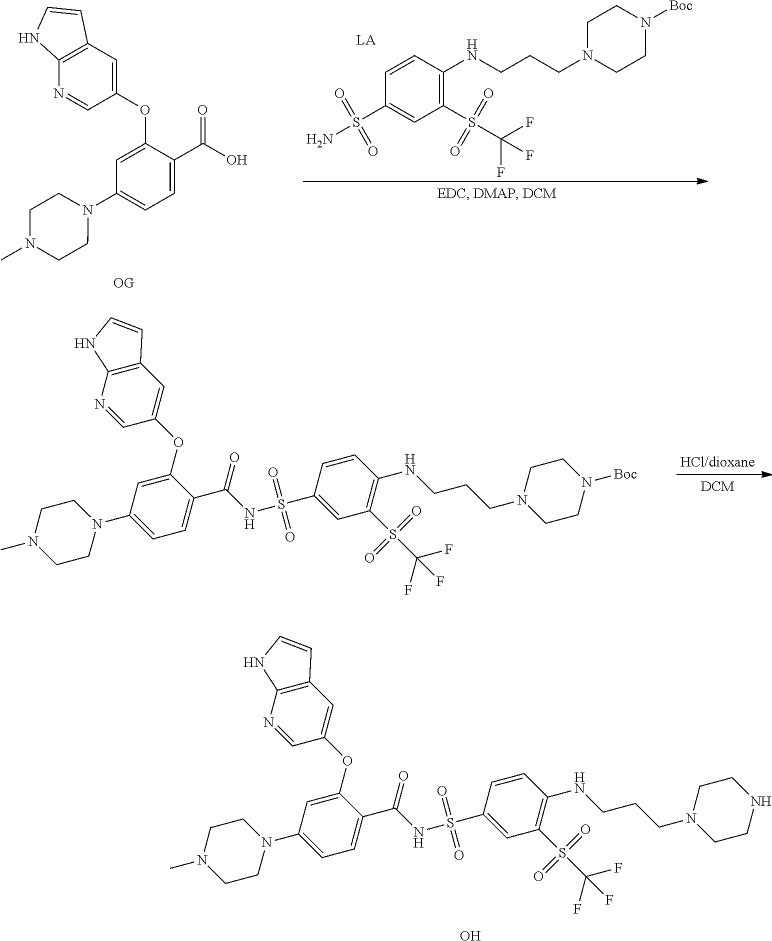 [SMILES: CN1CCN(c2ccc(C(=O)O)c(Oc3cnc4[nH]ccc4c3)c2)CC1 ; CC(C)(C)OC(=O)N1CCN(CCCNc2ccc(S(N)(=O)=O)cc2S(=O)(=O)C(F)(F)F)CC1 ; CN1CCN(c2ccc(C(=O)NS(=O)(=O)c3ccc(NCCCN4CCN(C(=O)OC(C)(C)C)CC4)c(S(=O)(=O)C(F)(F)F)c3)c(Oc3cnc4[nH]ccc4c3)c2)CC1 ; CN1CCN(c2ccc(C(=O)NS(=O)(=O)c3ccc(NCCCN4CCNCC4)c(S(=O)(=O)C(F)(F)F)c3)c(Oc3cnc4[nH]ccc4c3)c2)CC1 ; O]

### Step 1—Tert-butyl 4-(3-((4-(N-(2-((1H-pyrrolo[2,3-b]pyridin-5-yl)oxy)-4-(4-methylpiperazin-1-yl)benzoyl)sulfamoyl)-2-((trifluoromethyl)sulfonyl)phenyl)amino)propyl)piperazine-1-carboxylate

To a solution of 4-(4-methylpiperazin-1-yl)-2-(7H-pyrrolo[2,3-b]pyridin-5-yloxy)benzoic acid (500 mg, 1.42 mmol, Intermediate OG) in DCM (3 mL) was added EDC (550 mg, 3.55 mmol, 627 uL). DMAP (433 mg, 3.55 mmol) and tert-butyl 4-[3-[4-sulfamoyl-2-(trifluoromethylsulfonyl)anilino]propyl]piperazine-1-carboxylate (752 mg, 1.42 mmol, Intermediate LA). The mixture was then stirred at 25° C. for 2 hrs. On completion, the reaction mixture was concentrated under reduced pressure to give a residue. The residue was purified by prep-HPLC (basic condition) to give the title compound (1 g, 79% yield) as a white solid. LC-MS (ESI+) m/z 865.3 (M+H)⁺.

### Step 2—2-((1H-pyrrolo[2,3-b]pyridin-5-yl)oxy)-4-(4-methylpiperazin-1-yl)-N-((4-((3-(piperazin-1-yl)propyl)amino)-3-((trifluoromethyl)sulfonyl)phenyl)sulfonyl)benzamide

To a solution of tert-butyl 4-[3-[4-[[4-(4-methylpiperazin-1-yl)-2-(1H-pyrrolo[2,3-b]pyridin-5-yloxy)benzoyl]sulfamoyl]-2-(trifluoromethylsulfonyl)anilino]propyl]piperazine-1-carboxylate (100 mg, 115 μmol) in DCM (2 mL) was added HCl/dioxane (4 M, 28.9 uL). The mixture was then stirred at 25° C. for 1 hr. On completion, the reaction mixture was concentrated under reduced pressure to give the title compound (80 mg, HCl) as a white solid. LC-MS (ESI+) m/z 765.2 (M+H)⁺.

### Synthesis of Tert-butyl 4-(4′-chloro-2-formyl-[1,1′-biphenyl]-4-yl)piperazine-1-carboxylate (Intermediate OI)

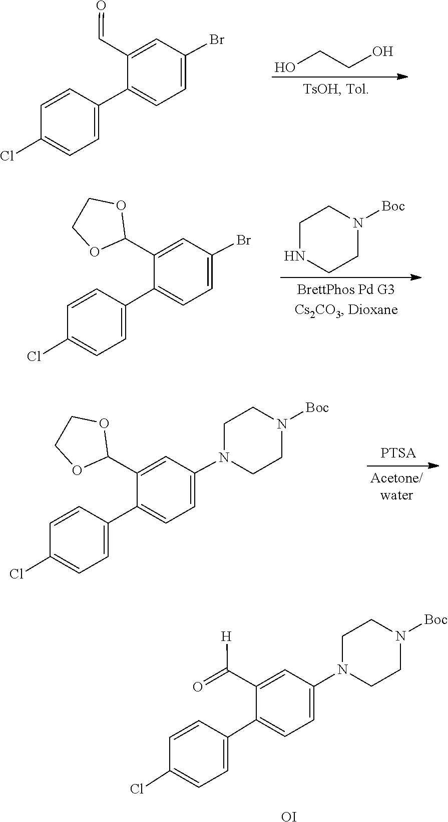

### Step 1—2-(4-Bromo-4′-chloro-[1,1′-biphenyl]-2-yl)-1,3-dioxolane

To a solution of 4-bromo-4′-chloro-[1,1′-biphenyl]-2-carbaldehyde (500 mg, 1.69 mmol, synthesized via Step 1 of Intermediate LD) and ethylene glycol (525 mg, 8.45 mmol) in toluene (5 mL) was added 4-methylbenzenesulfonic acid (29.1 mg, 169 μmol). The mixture was then stirred at 128° C. for 16 hours. On completion, the reaction mixture was quenched with water (10 mL) and extracted with ethyl acetate (2× 10 mL). The combined organic layers were washed with brine (20 mL) and dried over anhydrous sodium sulfate, filtered and the filtrate was concentrated in vacuo to give the title compound (3.2 g, 53% yield) as a white solid. ¹H NMR (400 MHz, DMSO-d₆) δ=7.77 (d, J=2.0 Hz, 1H), 7.67 (dd, J=2.0, 8.3 Hz, 1H), 7.53 (d, J=8.4 Hz, 2H), 7.43-7.38 (m, 2H), 7.26 (d, J=8.4 Hz, 1H), 5.51-5.47 (m, 1H), 4.12-4.06 (m, 2H), 3.90-3.85 (m, 2H).

### Step 2—Tert-butyl 4-(4′-chloro-2-(1,3-dioxolan-2-yl)-[1,1′-biphenyl]-4-yl)piperazine-1-carboxylate

A mixture of 2-(4-bromo-4′-chloro-[1,1′-biphenyl]-2-yl)-1,3-dioxolane (430 mg, 1.27 mmol), tert-butyl piperazine-1-carboxylate (235.82 mg, 1.27 mmol, CAS #143238-38-4), BrettPhos Pd G3 (114 mg, 126 μmol), and Cs₂CO₃ (825 mg, 2.53 mmol) in dioxane (20 mL) was degassed and purged with N₂ three times. Then the mixture was stirred at 70° C. for 7 hr under N₂ atmosphere. On completion, the reaction mixture was quenched with water (10 mL) and extracted by dichloromethane (2×10 mL). The combined organic layers were washed by brine (20 mL) and dried over anhydrous sodium sulfate, filtered and the filtrate was concentrated in vacuo to give the crude residue. The crude residue was purified by reversed-phase HPLC (0.1% FA condition) to give the title compound (170 mg, 30% yield) as a yellow solid. LC-MS (ESI⁺) m/z 446.1 (M+H)⁺.

### Step 3—Tert-butyl 4-(4′-chloro-2-formyl-[1,1′-biphenyl]-4-yl)piperazine-1-carboxylate

To a solution of tert-butyl 4-(4′-chloro-2-(1,3-dioxolan-2-yl)-[1,1′-biphenyl]-4-yl)piperazine-1-carboxylate (170 mg, 382 μmol) in acetone (4 mL) and Water (0.5 mL) was added paratoluene sulfonic acid (197 ug, 1.15 μmol, CAS #104-15-4). The mixture was stirred at 30° C. for 16 hrs. On completion, the reaction mixture was concentrated under reduced pressure to remove solvent. The residue was extracted with dichloromethane (2×10 mL). The combined organic layers were washed with brine (20 mL) and dried over anhydrous sodium sulfate, filtered and the filtrate was concentrated in vacuo to give the title compound (150 mg) as yellow oil. LC-MS (ESI⁺) m/z 423.1 (M+Na)⁺.

### Synthesis of 2-((1H-pyrrolo[2,3-b]pyridin-5-yl)oxy)-4-(4-((4-(4-(tert-butoxycarbonyl)piperazin-1-yl)-4′-chloro-[1,1′-biphenyl]-2-yl)methyl)piperazin-1-yl)benzoic Acid (Intermediate OJ)

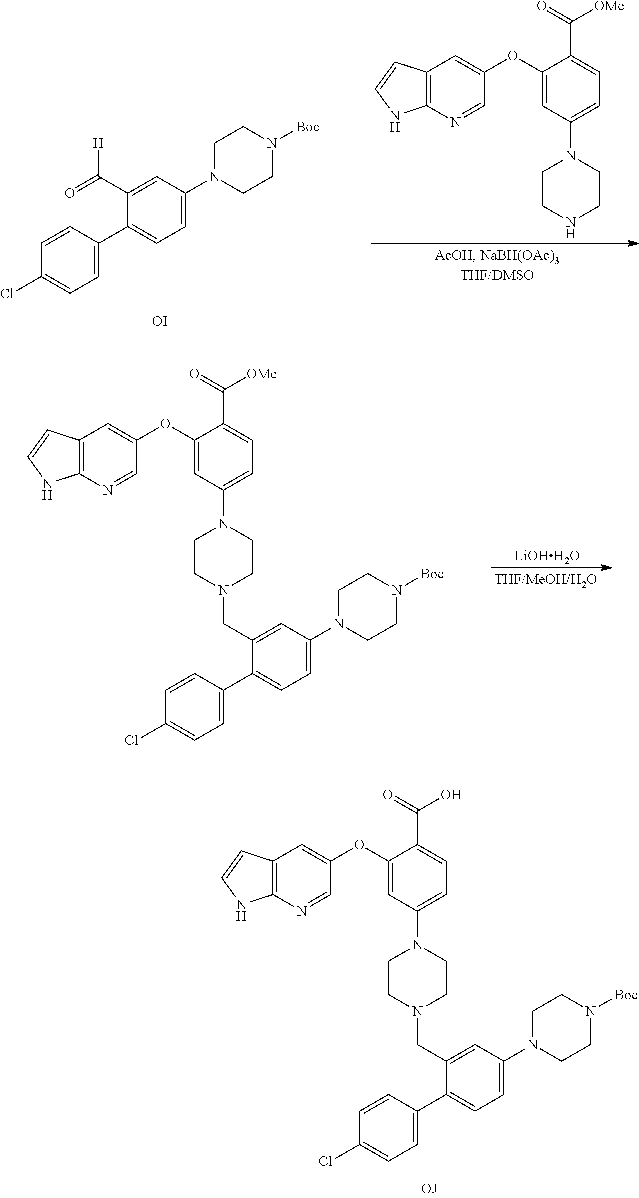 [SMILES: CC(C)(C)OC(=O)N1CCN(c2ccc(-c3ccc(Cl)cc3)c(C=O)c2)CC1 ; COC(=O)c1ccc(N2CCNCC2)cc1Oc1cnc2[nH]ccc2c1 ; "OI" ; COC(=O)c1ccc(N2CCN(Cc3cc(N4CCN(C(=O)OC(C)(C)C)CC4)ccc3-c3ccc(Cl)cc3)CC2)cc1Oc1cnc2[nH]ccc2c1 ; "LiOH·H2O" ; CC(C)(C)OC(=O)N1CCN(c2ccc(-c3ccc(Cl)cc3)c(CN3CCN(c4ccc(C(=O)O)c(Oc5cnc6[nH]ccc6c5)c4)CC3)c2)CC1]

### Step 1—Tert-butyl 4-(2-((4-(3-((1H-pyrrolo[2,3-b]pyridin-5-yl)oxy)-4-(methoxycarbonyl)phenyl)piperazin-1-yl)methyl)-4′-chloro-[1,1′-biphenyl]-4-yl)piperazine-1-carboxylate

Synthesis of To a solution of tert-butyl 4-(4′-chloro-2-formyl-[1,1′-biphenyl]-4-yl)piperazine-1-carboxylate (150 mg, 374 μmol, Intermediate OI) and methyl 2-((1H-pyrrolo[2,3-b]pyridin-5-yl)oxy)-4-(piperazin-1-yl)benzoate (131 mg, 374 μmol, synthesized via Step 1 of Intermediate NQ) in THF (1 mL) and DMSO (1 mL) was added AcOH (22.5 mg, 374 μmol, 21.4 uL) and 4 Å molecular sieves (100 mg). The mixture was then stirred at 25° C. for 5 min. Next, NaBH(OAc)₃ (79.3 mg, 374 μmol) was added and the reaction mixture was stirred at 25° C. for 12 hrs. On completion, the reaction mixture was filtered and concentrated under reduced pressure to give a residue. The residue was purified by reversed-phase HPLC (0.1% FA condition) to give the title compound (50 mg, 18% yield) as a white solid. LC-MS (ESI⁺) m/z 737.5 (M+H)⁺.

### Step 2—2-((1H-pyrrolo[2,3-b]pyridin-5-yl)oxy)-4-(4-((4-(4-(tert-butoxycarbonyl)piperazin-1-yl)-4′-chloro-[1,1′-biphenyl]-2-yl)methyl)piperazin-1-yl)benzoic Acid

To a solution of tert-butyl 4-(2-((4-(3-((1H-pyrrolo[2,3-b]pyridin-5-yl)oxy)-4-(methoxycarbonyl)phenyl)piperazin-1-yl)methyl)-4′-chloro-[1,1′-biphenyl]-4-yl)piperazine-1-carboxylate (80 mg, 108 μmol) in THF (4 mL), H₂O (1 mL) and MeOH (1 mL) was added LiOH·H₂O (22.7 mg, 542 μmol). The mixture was then stirred at 40° C. for 12 hrs. On completion, HCl (1N) was added to the reaction mixture until the pH was 5, then the mixture was diluted with water (10 mL) and extracted with dichloromethane (2×10 mL). The combined organic layers were dried over anhydrous sodium sulfate, filtered and the filtrate was concentrated in vacuo to give the title compound (80 mg. HCl) as white solid. LC-MS (ESI⁺) m/z 723.3 (M+H)⁺.

### Synthesis of 2-((1H-pyrrolo[2,3-b]pyridin-5-yl)oxy)-4-(4-((4′-chloro-4-(piperazin-1-yl)-[1,1′-biphenyl]-2-yl)methyl)piperazin-1-yl)-N-((4-((3-(dimethylamino)propyl)amino)-3-((trifluoromethyl)sulfonyl)phenyl)sulfonyl)benzamide (Intermediate OK)

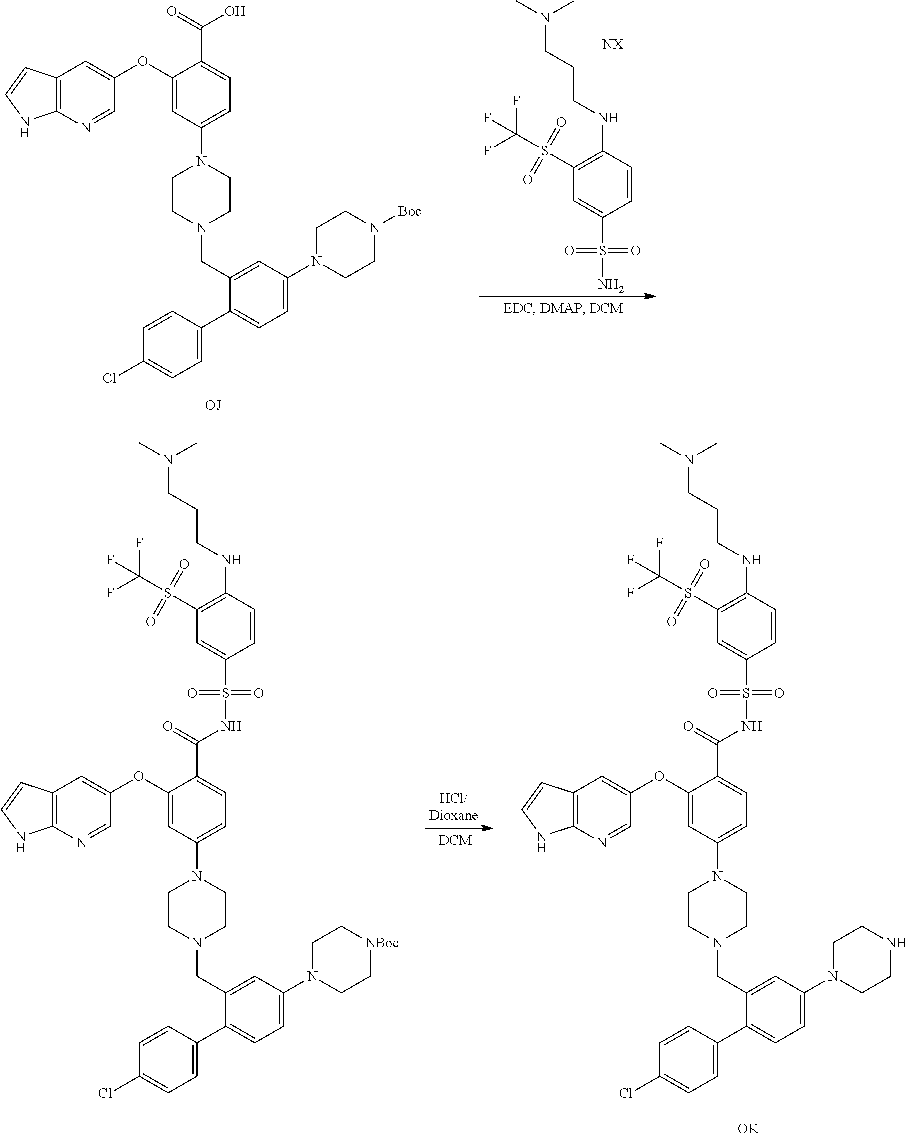 [SMILES: CC(C)(C)OC(=O)N1CCN(c2ccc(-c3ccc(Cl)cc3)c(CN3CCN(c4ccc(C(=O)O)c(Oc5cnc6[nH]ccc6c5)c4)CC3)c2)CC1 ; CN(C)CCCNc1ccc(S(N)(=O)=O)cc1S(=O)(=O)C(F)(F)F ; CN(C)CCCNc1ccc(S(=O)(=O)NC(=O)c2ccc(N3CCN(Cc4cc(N5CCN(C(=O)OC(C)(C)C)CC5)ccc4-c4ccc(Cl)cc4)CC3)cc2Oc2cnc3[nH]ccc3c2)cc1S(=O)(=O)C(F)(F)F ; CN(C)CCCNc1ccc(S(=O)(=O)NC(=O)c2ccc(N3CCN(Cc4cc(N5CCNCC5)ccc4-c4ccc(Cl)cc4)CC3)cc2Oc2cnc3[nH]ccc3c2)cc1S(=O)(=O)C(F)(F)F ; "OK"]

### Step 1—Tert-butyl 4-(2-((4-(3-((1H-pyrrolo[2,3-b]pyridin-5-yl)oxy)-4-(((4-((3-(dimethylamino)propyl)amino)-3-((trifluoromethyl)sulfonyl)phenyl)sulfonyl)carbamoyl)phenyl)piperazin-1-yl)methyl)-4′-chloro-[1,1′-biphenyl]-4-yl)piperazine-1-carboxylate

To a solution of 2-((1H-pyrrolo[2,3-b]pyridin-5-yl)oxy)-4-(4-((4-(4-(tert-butoxycarbonyl)piperazin-1-yl)-4′-chloro-[1,1′-biphenyl]-2-yl)methyl)piperazin-1-yl)benzoic acid (80 mg, 105 μmol, HCl, Intermediate OJ) in DCM (5 mL) was added EDC (32.6 mg, 210 μmol, 37.2 uL). Then the mixture was stirred for 30 min, 4-((3-(dimethylamino)propyl)amino)-3-((trifluoromethyl)sulfonyl)benzenesulfonamide (41.0 mg, 105 μmol, Intermediate NX) and DMAP (32.1 mg, 263 μmol) was added. The mixture was then stirred at 25° C. for 2 hrs. On completion, the reaction mixture was concentrated under reduced pressure to remove solvent and purified by reversed-phase HPLC (0.1% FA condition) to give the title compound (90 mg, 78 μmol, 75% yield, FA) as a white solid. LC-MS (ESI⁺) m/z 1096.4 (M+H)⁺.

### Step 2—2-((1H-pyrrolo[2,3-b]pyridin-5-yl)oxy)-4-(4-((4′-chloro-4-(piperazin-1-yl)-[1,1′-biphenyl]-2-yl)methyl)piperazin-1-yl)-N-((4-((3-(dimethylamino)propyl)amino)-3-((trifluoromethyl)sulfonyl)phenyl)sulfonyl)benzamide

To a solution of tert-butyl 4-(2-((4-(3-((1H-pyrrolo[2,3-b]pyridin-5-yl)oxy)-4-(((4-((3-(dimethylamino)propyl)amino)-3-((trifluoromethyl)sulfonyl)phenyl)sulfonyl)carbamoyl)phenyl)piperazin-1-yl)methyl)-4′-chloro-[1,1′-biphenyl]-4-yl)piperazine-1-carboxylate (90 mg, 78.9 μmol, FA) in DCM (8 mL) was added HCl/dioxane (4 M, 1.08 mL). The mixture was stirred at 25° C. for 1 hour. On completion, the reaction mixture was concentrated under reduced pressure to remove solvent to give the title compound (80 mg, crude, HCl) as yellow solid. LC-MS (ESI⁺) m/z 996.0 (M+H)⁺.

### Synthesis of 4-(4-((4′-Chloro-5,5-dimethyl-3,4,5,6-tetrahydro-[1,1′-biphenyl]-2-yl)methyl)piperazin-1-yl)benzoic Acid (Intermediate OL)

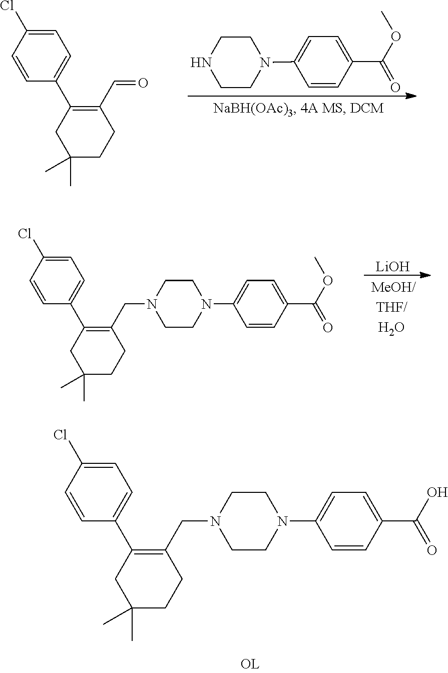

### Step 1—Methyl 4-(4-((4′-chloro-5,5-dimethyl-3,4,5,6-tetrahydro-[1,1′-biphenyl]-2-yl)methyl)piperazin-1-yl)benzoate

To a solution of 4′-chloro-5,5-dimethyl-3,4,5,6-tetrahydro-[1,1′-biphenyl]-2-carbaldehyde (2.00 g, 8.04 mmol, CAS #1228837 May 5) in DCM (30 mL) was added 4 Å molecular sieves (4 g) and methyl 4-(piperazin-1-yl)benzoate (2.66 g, 12.1 mmol, CAS #163210-97-7). The mixture was then stirred at 20° C. for 1 hr, then NaBH(OAc)₃ (3.41 g, 16.1 mmol) was added and the mixture was stirred at 20° C. for 1 hr. On completion, the mixture was filtered and organic phase was diluted with H₂O (50 mL), and extracted with DCM (30 mL×3). The combined organic layer was dried over anhydrous Na₂SO₄, filtered to give a residue and purified by column chromatography (SiO₂, Petroleum ether/Ethyl acetate=10/1 to 3/1) to give the title compound (2.7 g, 70% yield) as a white solid. LC-MS (ESI⁺) m/z 453.3 (M+H)⁺.

### Step 2—4-(4-((4′-Chloro-5,5-dimethyl-3,4,5,6-tetrahydro-[1,1′-biphenyl]-2-yl)methyl)piperazin-1-yl)benzoic Acid

To a solution of methyl 4-(4-((4′-chloro-5,5-dimethyl-3,4,5,6-tetrahydro-[1,1′-biphenyl]-2-yl)methyl)piperazin-1-yl)benzoate (2.7 g, 6.0 mmol) in THF (10 mL), MeOH (10 mL) and H₂O (4 mL) was added LiOH·H₂O (1.25 g, 29.8 mmol). The mixture was then stirred at 50° C. for 12 hrs. On completion, the mixture was concentrated to remove organic solvent and the pH was adjusted to 4 by adding 1M HCl solution, then extracted with DCM (150 mL×3). The combined organic layer was dried over anhydrous Na₂SO₄, filtered and concentrated to give the title compound (2.6 g) as a light yellow solid. LC-MS (ESI⁺) m/z 439.3 (M+H)⁺.

### Synthesis of (R)-4-(4-((4′-chloro-5,5-dimethyl-3,4,5,6-tetrahydro-[1,1′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide (Intermediate OM)

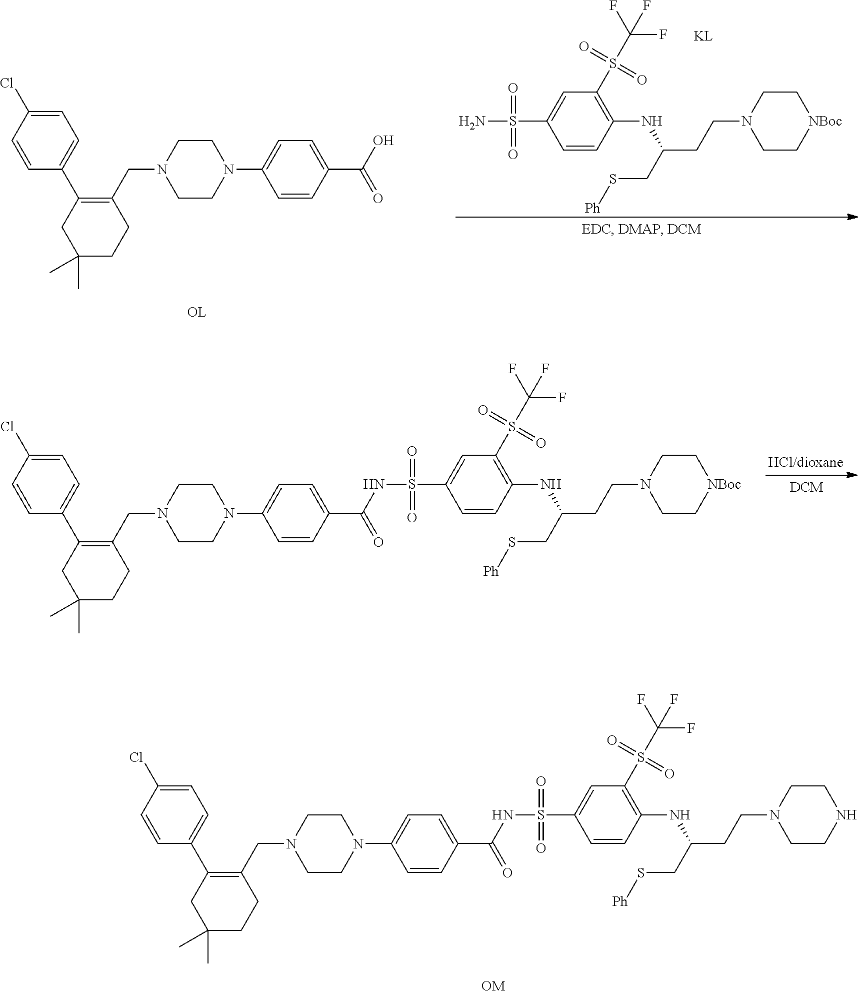 [SMILES: CC1(C)CCC(CN2CCN(c3ccc(C(=O)O)cc3)CC2)=C(c2ccc(Cl)cc2)C1 ; CC(C)(C)OC(=O)N1CCN(CC[C@H](CSc2ccccc2)Nc2ccc(S(N)(=O)=O)cc2S(=O)(=O)C(F)(F)F)CC1 ; CC1(C)CCC(CN2CCN(c3ccc(C(=O)NS(=O)(=O)c4ccc(N[C@H](CCN5CCN(C(=O)OC(C)(C)C)CC5)CSc5ccccc5)c(S(=O)(=O)C(F)(F)F)c4)cc3)CC2)=C(c2ccc(Cl)cc2)C1 ; CC1(C)CCC(CN2CCN(c3ccc(C(=O)NS(=O)(=O)c4ccc(N[C@H](CCN5CCNCC5)CSc5ccccc5)c(S(=O)(=O)C(F)(F)F)c4)cc3)CC2)=C(c2ccc(Cl)cc2)C1]

### Step 1—(R)-tert-butyl 4-(3-((4-(N-(4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carboxylate

To a solution of 4-(4-((4′-chloro-5,5-dimethyl-3,4,5,6-tetrahydro-[1,1′-biphenyl]-2-yl)methyl)piperazin-1-yl)benzoic acid (672 mg, 1.53 mmol, Intermediate OL) in DCM (15 mL) was added EDC (475.03 mg, 3.06 mmol) and DMAP (467 mg, 3.83 mmol). Next, (R)-tert-butyl 4-(4-(phenylthio)-3-((4-sulfamoyl-2-((trifluoromethyl)sulfonyl)phenyl)amino)butyl)piperazine-1-carboxylate (1 g, 1.53 mmol, Intermediate KL) was added and the mixture was stirred at 25° C. for 12 hrs. On completion, the mixture was concentrated to give a residue and purified by reversed-phase HPLC (0.1% FA condition) to give the title compound (1.1 g, 66% yield) as a light yellow solid. LC-MS (ESI⁺) m/z 537.7 (M/2+H)⁺.

### Step 2—(R)-4-(4-((4′-chloro-5,5-dimethyl-3,4,5,6-tetrahydro-[1,1′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide

To a solution of (R)-tert-butyl 4-(3-((4-(N-(4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carboxylate (1.00 g, 931 μmol) in DCM (4 mL) was added HCl/dioxane (4 M, 5.00 mL). The mixture was then stirred at 20° C. for 2 hrs. On completion, the mixture was concentrated to give the title compound (1 g, HCl) as a white solid. LC-MS (ESI⁺) m/z 973.4 (M+H)⁺.

### Synthesis of Tert-butyl (R)-4-(4′-chloro-2-(hydroxy(1-(4-(methoxycarbonyl)phenyl)piperidin-4-yl)methyl)-[1,1′-biphenyl]-4-yl)piperazine-1-carboxylate (Intermediate ON) and tert-butyl (S)-4-(4′-chloro-2-(hydroxy(1-(4-(methoxycarbonyl)phenyl)piperidin-4-yl)methyl)-[1,1′-biphenyl]-4-yl)piperazine-1-carboxylate (Intermediate 00)

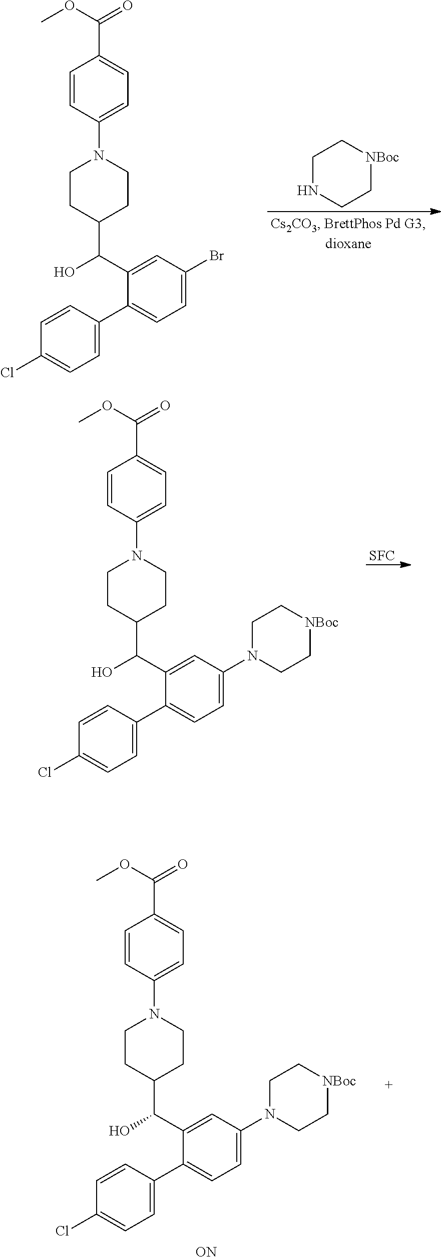 [SMILES: COC(=O)c1ccc(N2CCC(C(O)c3cc(Br)ccc3-c3ccc(Cl)cc3)CC2)cc1 ; CC(C)(C)OC(=O)N1CCNCC1 ; COC(=O)c1ccc(N2CCC(C(O)c3cc(N4CCN(C(=O)OC(C)(C)C)CC4)ccc3-c3ccc(Cl)cc3)CC2)cc1 ; "SFC" ; COC(=O)c1ccc(N2CCC([C@@H](O)c3cc(N4CCN(C(=O)OC(C)(C)C)CC4)ccc3-c3ccc(Cl)cc3)CC2)cc1 ; "ON"]

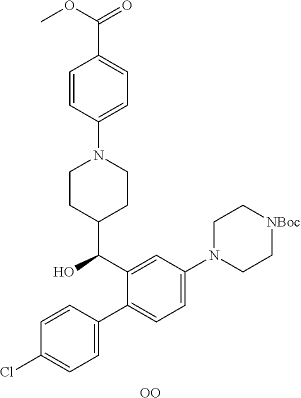 [SMILES: COC(=O)c1ccc(N2CCC([C@H](O)c3cc(N4CCN(C(=O)OC(C)(C)C)CC4)ccc3-c3ccc(Cl)cc3)CC2)cc1 ; "OO"]

### Step 1—Tert-butyl 4-(4′-chloro-2-(hydroxy(1-(4-(methoxycarbonyl)phenyl)piperidin-4-yl)methyl)-[1,1′-biphenyl]-4-yl)piperazine-1-carboxylate

A mixture of methyl 4-(4-((4-bromo-4′-chloro-[1,1′-biphenyl]-2-yl)(hydroxy)methyl)piperidin-1-yl)benzoate (1 g, 1.94 mmol, synthesized via Steps 1˜4 of Intermediate KU), tert-butyl piperazine-1-carboxylate (724 mg, 3.88 mmol, CAS #143238-38-4), BrettPhos Pd G3 (264 mg, 291 μmol, CAS #1470372-59-8), and Cs₂CO₃ (1.27 g, 3.88 mmol) in dioxane (10 mL) was degassed and purged with N₂ for three times. Then the mixture was stirred at 60° C. for 8 hrs under N₂ atmosphere. On completion, the reaction mixture was poured into water (30 mL) and extracted with ethyl acetate (30 mL×3). The combined organic layers were dried over anhydrous sodium sulfate, filtered and the filtrate was concentrated in vacuo to give the crude residue. The residue was purified by column chromatography (SiO₂, Petroleum ether/Ethyl acetate=10/1 to 3/1) to give the title compound (600 mg, 47% yield) as a white solid. LC-MS (ESI⁺) m/z 620.3 (M+H)⁺.

### Step 2—Tert-butyl (R)-4-(4′-chloro-2-(hydroxy(1-(4-(methoxycarbonyl)phenyl)piperidin-4-yl)methyl)-[1,1′-biphenyl]-4-yl)piperazine-1-carboxylate and tert-butyl (S)-4-(4′-chloro-2-(hydroxy(1-(4-(methoxycarbonyl)phenyl)piperidin-4-yl)methyl)-[1,1′-biphenyl]-4-yl)piperazine-1-carboxylate

Tert-butyl 4-(4′-chloro-2-(hydroxy(1-(4-(methoxycarbonyl)phenyl)piperidin-4-yl)methyl)-[1,1′-biphenyl]-4-yl)piperazine-1-carboxylate (650 mg) in MeOH (2 mL) was purified by reversed-phase HPLC (column: DAICEL CHIRALPAK AD (250 mm×30 mm, 10 um); mobile phase: [EtOH/ACN]; B %: 50%-50%, A3.6; 100 min) to give tert-butyl (R)-4-(4′-chloro-2-(hydroxy(1-(4-(methoxycarbonyl)phenyl)piperidin-4-yl)methyl)-[1,1′-biphenyl]-4-yl)piperazine-1-carboxylate (260 mg, 70% yield) and tert-butyl (S)-4-(4′-chloro-2-(hydroxy(1-(4-(methoxycarbonyl)phenyl)piperidin-4-yl)methyl)-[1,1′-biphenyl]-4-yl)piperazine-1-carboxylate (40 mg, 26% yield) as a white solids. LC-MS (ESI⁺) m/z 620.5 (M+H)⁺ for both isomers. Absolute stereochemistry of the enantiomers was assigned arbitrarily.

### Synthesis of Tert-butyl 4-((R)-3-((4-(N-(4-((R)-3-(dimethylamino)pyrrolidin-1-yl)benzoyl)sulfamoyl)-2-((trifluoromethyl)sulfonyl)phenyl)amino)-4-(phenylthio)butyl)piperazine-1-carboxylate (Intermediate OP)

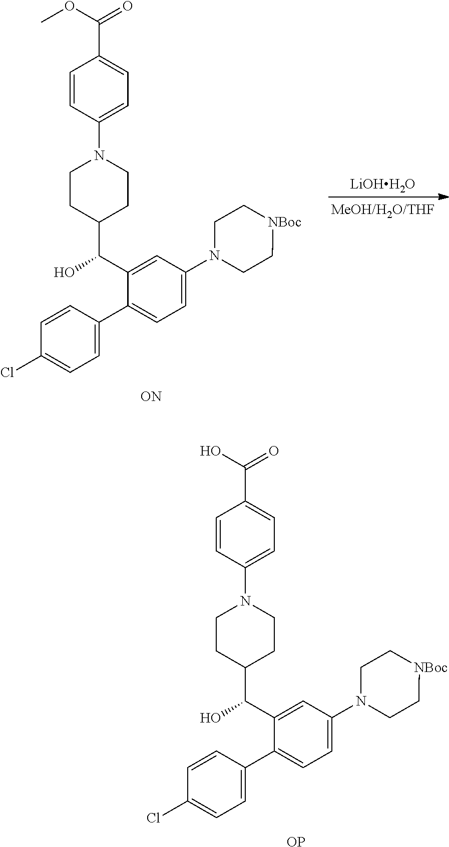 [SMILES: COC(=O)c1ccc(N2CCC([C@@H](O)c3cc(N4CCN(C(=O)OC(C)(C)C)CC4)ccc3-c3ccc(Cl)cc3)CC2)cc1 ; "LiOH·H2O" ; "ON" ; CC(C)(C)OC(=O)N1CCN(c2ccc(-c3ccc(Cl)cc3)c([C@H](O)C3CCN(c4ccc(C(=O)O)cc4)CC3)c2)CC1 ; "OP"]

To a solution of (R)-tert-butyl 4-(4′-chloro-2-(hydroxy(1-(4-(methoxycarbonyl)phenyl)piperidin-4-yl)methyl)-[1,1′-biphenyl]-4-yl)piperazine-1-carboxylate (260 mg, 419 μmol, Intermediate ON) in MeOH (0.5 mL), THF (2 mL) and H₂O (0.5 mL) was added LiOH·H₂O (88.0 mg, 2.10 mmol). The mixture was stirred at 50° C. for 12 hrs. On completion, the reaction mixture was added HCl (1N) until the pH=4, then the mixture was diluted with water (2 mL) and extracted with dichloromethane (5×3 mL). The combined organic layers were dried over anhydrous sodium sulfate, filtered and the filtrate was concentrated in vacuo to give the title compound (250 mg) as a white solid. LC-MS (ESI⁺) m/z 606.2 (M+H)⁺.

### Synthesis of 4-(4-((R)-(4′-chloro-4-(piperazin-1-yl)-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide (Intermediate OQ)

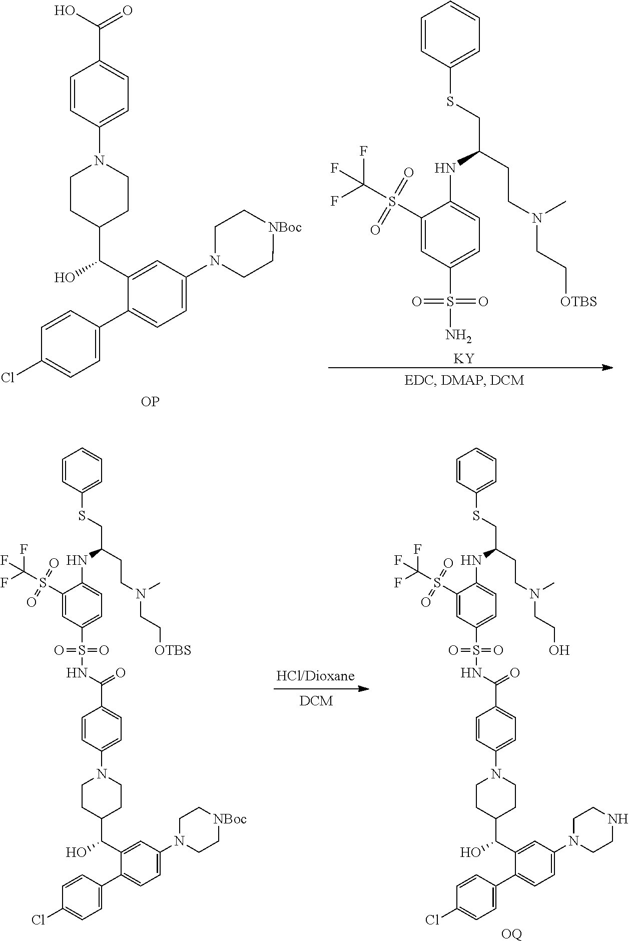

### Step 1—Tert-butyl 4-(2-((R)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)piperazine-1-carboxylate

To a solution of (R)-4-(4-((4-(4-(tert-butoxycarbonyl)piperazin-1-yl)-4′-chloro-[1,1′-biphenyl]-2-yl)(hydroxy)methyl)piperidin-1-yl)benzoic acid (250 mg, 412 μmol, Intermediate OP) in DCM (4 mL) was added (R)-4-((4-((2-((tert-butyldimethylsilyl)oxy)ethyl)(methyl)amino)-1-(phenylthio)butan-2-yl)amino)-3-((trifluoromethyl)sulfonyl)benzenesulfonamide (271 mg, 412 μmol, Intermediate KY), EDC (160 mg, 1.03 mmol) and DMAP (126 mg, 1.03 mmol). The mixture was then stirred at 25° C. for 2 hrs. On completion, the reaction mixture was filtered to give a filtrate. The filtrate was purified by reversed-phase HPLC (0.1% FA condition) to give the title compound (180 mg, 32% yield) as a white solid. LC-MS (ESI⁺) m/z 1244.0 (M+H)⁺.

### Step 2—4-(4-((R)-(4′-chloro-4-(piperazin-1-yl)-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide

To a solution of tert-butyl 4-(2-((R)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)piperazine-1-carboxylate (160 mg, 129 μmol) in DCM (5 mL) was added HCl/dioxane (4 M, 1 mL). The mixture was then stirred at 25° C. for 2 hrs. On completion, the reaction mixture was concentrated under reduced pressure to give the title compound (180 mg, HCl) as a white solid. LC-MS (ESI⁺) m/z 1029.2 (M+H)⁺.

### Synthesis of (S)-4-(4-((4-(4-(tert-butoxycarbonyl)piperazin-1-yl)-4′-chloro-[1,1′-biphenyl]-2-yl)(hydroxy)methyl)piperidin-1-yl)benzoic Acid (Intermediate OR)

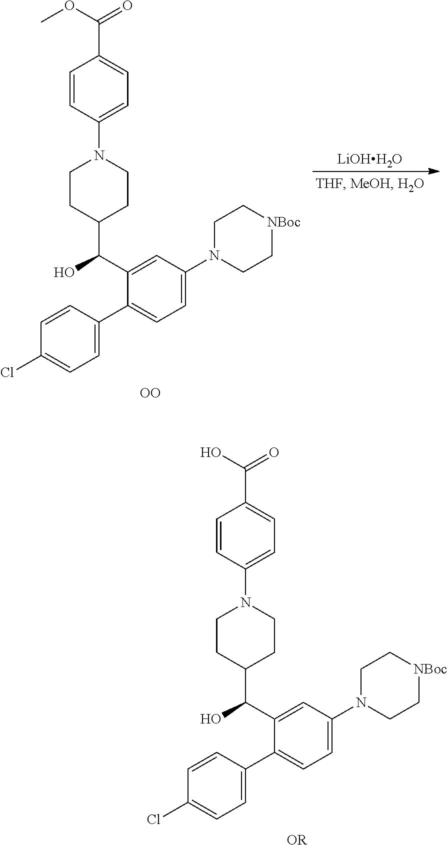 [SMILES: COC(=O)c1ccc(N2CCC([C@H](O)c3cc(N4CCN(C(=O)OC(C)(C)C)CC4)ccc3-c3ccc(Cl)cc3)CC2)cc1 ; "LiOH·H2O" ; "OO" ; CC(C)(C)OC(=O)N1CCN(c2ccc(-c3ccc(Cl)cc3)c([C@@H](O)C3CCN(c4ccc(C(=O)O)cc4)CC3)c2)CC1 ; "OR"]

To a solution of tert-butyl (S)-4-(4′-chloro-2-(hydroxy(1-(4-(methoxycarbonyl)phenyl)piperidin-4-yl)methyl)-[1,1′-biphenyl]-4-yl)piperazine-1-carboxylate (260 mg, 419 μmol, Intermediate OO) in THF (2 mL), MeOH (0.5 mL) and H₂O (0.5 mL) was added LiOH·H₂O (87.9 mg, 2.1 mmol). The mixture was then stirred at 50° C. for 12 hrs. On completion, the reaction mixture was concentrated in vacuo to remove solvent, then the reaction mixture was diluted with water (10 mL) and extracted by dichloromethane (3×10 mL). The combined organic layers were washed by brine (30 mL) and dried over anhydrous sodium sulfate, filtered and the filtrate was concentrated in vacuo to give the title compound (240 mg) as a white solid. LC-MS (ESI⁺) m/z 606.5 (M+H)⁺.

### Synthesis of 4-(4-((S)-(4′-chloro-4-(piperazin-1-yl)-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide (Intermediate OS)

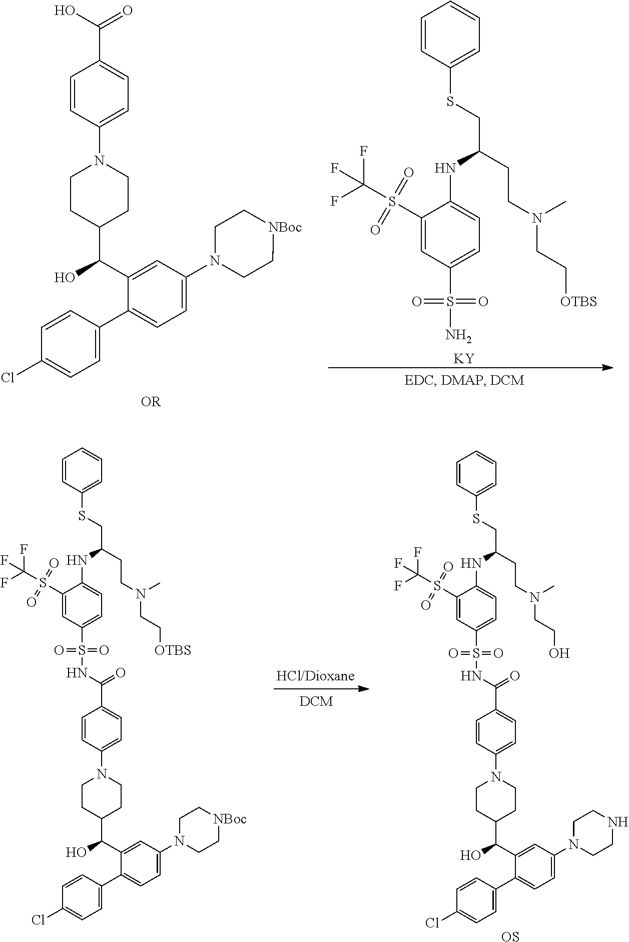

### Step 1—Tert-butyl 4-(2-((S)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)piperazine-1-carboxylate

To a solution of (S)-4-(4-((4-(4-(tert-butoxycarbonyl)piperazin-1-yl)-4′-chloro-[1,1′-biphenyl]-2-yl)(hydroxy)methyl)piperidin-1-yl)benzoic acid (220 mg, 362.9 μmol, Intermediate OR) in DCM (2 mL) was added DMAP (111 mg, 907 μmol), EDC (113 mg, 726 μmol) and (R)-4-((4-((2-((tert-butyldimethylsilyl)oxy)ethyl)(methyl)amino)-1-(phenylthio)butan-2-yl)amino)-3-((trifluoromethyl)sulfonyl)benzenesulfonamide (238 mg, 363 μmol, Intermediate KY). The mixture was then stirred at 30° C. for 3 hrs. On completion, the reaction mixture was concentrated in vacuo to remove solvent. Then the crude residue was purified by reversed-phase HPLC (0.1% FA condition) to give the title compound as a white solid (340 mg, 72% yield, FA) as a white solid. LC-MS (ESI⁺) m/z 1244.0 (M+H)⁺.

### Step 2—4-(4-((S)-(4′-chloro-4-(piperazin-1-yl)-[1,1′-biphenyl]-2-yl)(hydroxy)methyl)piperidin-1-yl)-N-((4-(((R)-4-((2-hydroxyethyl)(methyl)amino)-1-(phenylthio)butan-2-yl)amino)-3-((trifluoromethyl)sulfonyl)phenyl)sulfonyl)benzamide

To a solution of tert-butyl 4-(2-((S)-(1-(4-(((4-(((R)-4-((2-((tert-butyldimethylsilyl)oxy)ethyl)(methyl)amino)-1-(phenylthio)butan-2-yl)amino)-3-((trifluoromethyl)sulfonyl)phenyl)sulfonyl)carbamoyl)phenyl)piperidin-4-yl)(hydroxy)methyl)-4′-chloro-[1,1′-biphenyl]-4-yl)piperazine-1-carboxylate (340 mg, 264 μmol) in DCM (4 mL) was added HCl/dioxane (4 M, 1 mL). The mixture was then stirred at 25° C. for 2 hrs. On completion, the reaction mixture was concentrated in vacuo to give the title compound (260 mg) as a white solid. LC-MS (ESI⁺) m/z 1029.3 (M+H)⁺.

### Synthesis of Tert-butyl (S)-(4-(phenylthio)-3-((4-sulfamoyl-2-((trifluoromethyl)sulfonyl)phenyl)amino)butyl)carbamate (Intermediate OT)

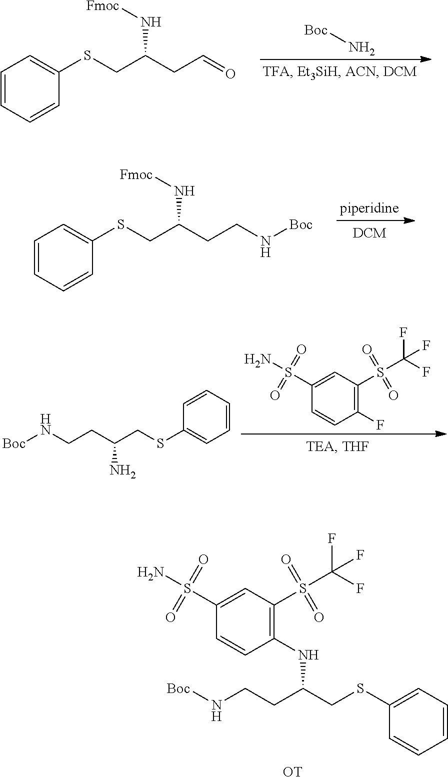

### Step 1—(9H-fluoren-9-yl)methyl tert-butyl (4-(phenylthio)butane-1,3-diyl)(R)-dicarbamate

To a solution of (9H-fluoren-9-yl)methyl (R)-(4-oxo-1-(phenylthio)butan-2-yl)carbamate (2.4 g, 5.75 mmol, synthesized via Steps 1-3 of Intermediate KK) in DCM (5 mL) and MeCN (15 mL) was added tert-butyl carbamate (2.02 g, 17.24 mmol, CAS #4248-19-5), Et₃SiH (3.34 g, 28.7 mmol, 4.59 mL) and TFA (1.97 g, 17.2 mmol, 1.28 mL) at 0° C. then the reaction was stirred at 25° C. for 12 hrs. On completion, the reaction mixture was quenched with H₂O (20 mL) at 25° C., and then extracted with DCM (3×50 mL). The combined organic layers were washed with brine (2×100 mL), dried over anhydrous sodium sulfate, filtered and concentrated under reduced pressure to give a residue. The residue was purified by column chromatography (SiO₂, Petroleum ether/Ethyl acetate=1/0 to 5/1) to give the title compound (1.3 g, 41% yield) as a yellow solid. LC-MS (ESI⁺) m/z 419.1. (M+H)⁺.

### Step 2—Tert-butyl (R)-(3-amino-4-(phenylthio)butyl)carbamate

To a solution of (9H-fluoren-9-yl)methyl tert-butyl (4-(phenylthio)butane-1,3-diyl) (R)-dicarbamate (1.3 g, 2.51 mmol) in DCM (4 mL) was added piperidine (2.56 g, 30.1 mmol, 2.97 mL). The reaction was stirred at 25° C. for 2 hrs. On completion, the reaction mixture was concentrated under reduced pressure to give a residue. The residue was purified by prep-HPLC (0.1% FA condition) to give the title compound (370 mg, 47% yield) as a yellow oil. LC-MS (ESI⁺) m/z 297.2. (M+H)⁺.

### Step 3—Tert-butyl (S)-(4-(phenylthio)-3-((4-sulfamoyl-2-((trifluoromethyl)sulfonyl)phenyl)amino)butyl)carbamate

To a solution of tert-butyl tert-butyl (R)-(3-amino-4-(phenylthio)butyl)carbamate (370 mg, 1.25 mmol) in ACN (8 mL) was added DIEA (807 mg, 6.24 mmol, 1.09 mL) and 4-fluoro-3-((trifluoromethyl)sulfonyl)benzenesulfonamide (575 mg, 1.87 mmol, CAS #1027345-08-9). The mixture was then stirred at 80° C. for 12 hrs. On completion, the reaction mixture was concentrated under reduced pressure to give a residue. The residue was purified by prep-HPLC (0.8 g/L ammonium bicarbonate) to give the title compound (560 mg, 75% yield) as a white solid. LC-MS (ESI⁺) m/z 583.3. (M+H)⁺.

### Synthesis of N-((4-(((R)-4-amino-1-(phenylthio)butan-2-yl)amino)-3-((trifluoromethyl)sulfonyl)phenyl)sulfonyl)-4-(4-((R)-(4′-chloro-[1,1′-biphenyl]-2-yl)(hydroxy)methyl)piperidin-1-yl)benzamide (Intermediate OU)

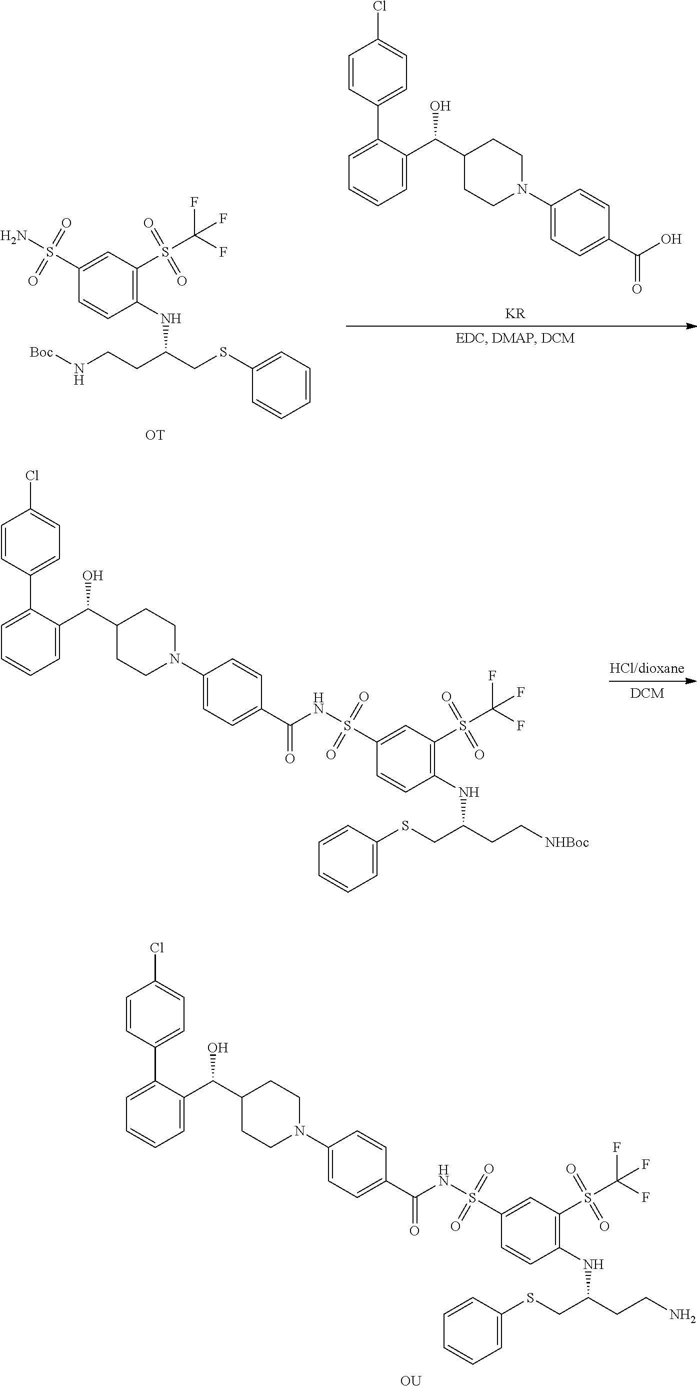

### Step 1—Tert-butyl ((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)carbamate

To a solution of (R)-4-(4-((4′-chloro-[1,1′-biphenyl]-2-yl)(hydroxy)methyl)piperidin-1-yl)benzoic acid (80 mg, 190 μmol, Intermediate KR) in DCM (1 mL) was added DMAP (23.2 mg, 190 μmol), CMPI (72.7 mg, 284 μmol), TEA (57.6 mg, 569 μmol, 79.2 uL) and tert-butyl (S)-(4-(phenylthio)-3-((4-sulfamoyl-2-((trifluoromethyl)sulfonyl)phenyl)amino)butyl)carbamate (111 mg, 190 μmol, Intermediate OT). The reaction was then stirred at 25° C. for 2 hrs. On completion, the reaction mixture was concentrated under reduced pressure to give a residue. The residue was purified by prep-HPLC (0.8 g/L ammonium bicarbonate) to give the title compound (90 mg, 48% yield) as a yellow solid. LC-MS (ESI⁺) m/z 987.7. (M+H)⁺.

### Step 2—N-((4-(((R)-4-amino-1-(phenylthio)butan-2-yl)amino)-3-((trifluoromethyl)sulfonyl)phenyl)sulfonyl)-4-(4-((R)-(4′-chloro-[1,1′-biphenyl]-2-yl)(hydroxy)methyl)piperidin-1-yl)benzamide

To a solution of tert-butyl ((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)carbamate (90 mg, 91.1 μmol) in DCM (2 mL) was added HCl/dioxane (4 M, 22.8 uL). The reaction was then stirred at 25° C. for 30 mins. On completion, the reaction mixture was concentrated under reduced pressure to give the title compound (85 mg) as a white solid. LC-MS (ESI⁺) m/z 888.2. (M+H)⁺.

### Synthesis of N-((4-(((R)-4-amino-1-(phenylthio)butan-2-yl)amino)-3-((trifluoromethyl)sulfonyl)phenyl)sulfonyl)-4-(4-((S)-(4′-chloro-[1,1′-biphenyl]-2-yl)(hydroxy)methyl)piperidin-1-yl)benzamide (Intermediate OV)

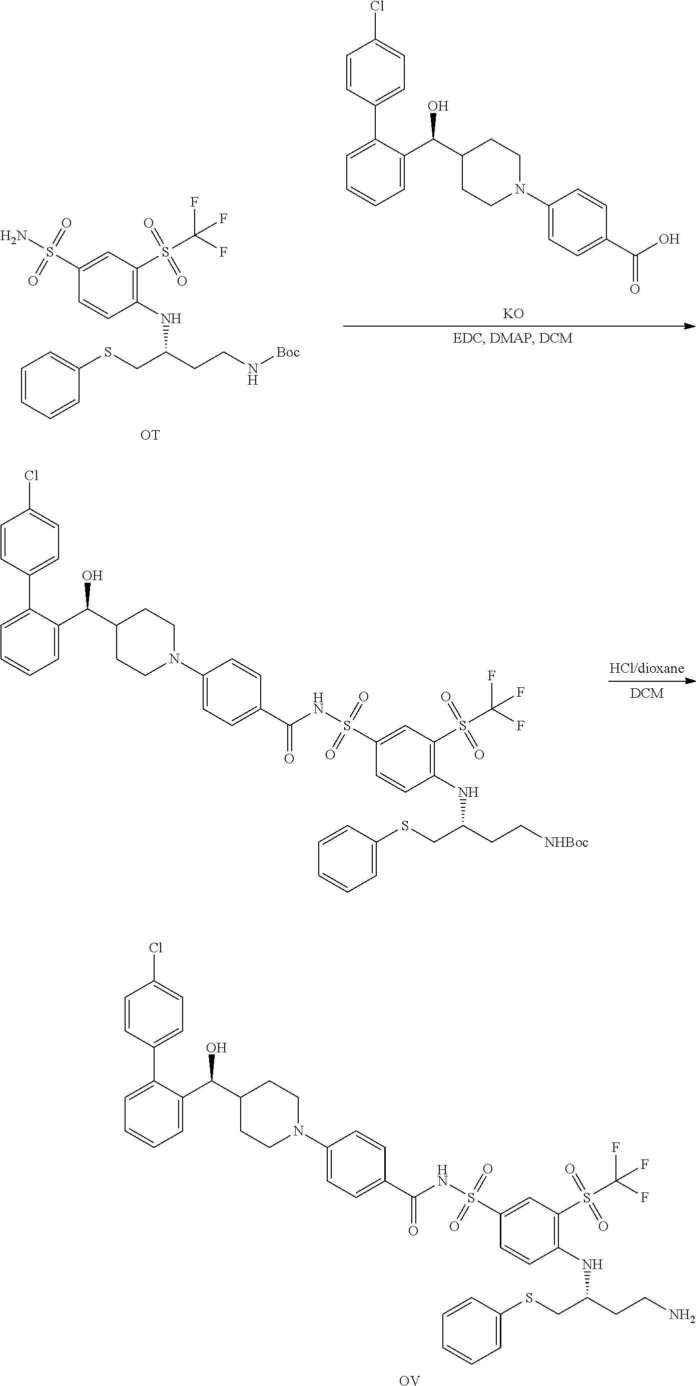

### Step 1—Tert-butyl ((R)-3-((4-(N-(4-(4-((S)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)carbamate

To a solution of (S)-4-(4-((4′-chloro-[1,1′-biphenyl]-2-yl)(hydroxy)methyl)piperidin-1-yl)benzoic acid (80 mg, 189 μmol, Intermediate KO) in DCM (2 mL) was added DMAP (23.1 mg, 189 μmol) and TEA (57.6 mg, 569 μmol), CMPI (121 mg, 474 μmol) and tert-butyl (R)-(4-(phenylthio)-3-((4-sulfamoyl-2-((trifluoromethyl)sulfonyl)phenyl)amino)butyl)carbamate (111 mg, 190 μmol, Intermediate OT). The mixture was then stirred at 25° C. for 1 hr. On completion, the reaction mixture was concentrated in vacuo to remove solvent. Then the crude residue was purified by reversed-phase HPLC (0.1% FA condition) to give the title compound (90 mg, 48% yield, FA) as a white solid. LC-MS (ESI+) m/z 987.6 (M+H)⁺.

### Step 2—N-((4-(((R)-4-amino-1-(phenylthio)butan-2-yl)amino)-3-((trifluoromethyl)sulfonyl)phenyl)sulfonyl)-4-(4-((S)-(4′-chloro-[1,1′-biphenyl]-2-yl)(hydroxy)methyl)piperidin-1-yl)benzamide

To a solution of tert-butyl ((R)-3-((4-(N-(4-(4-((S)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)carbamate (90 mg, 91.1 μmol) in DCM (2 mL) was added HCl/dioxane (4 M, 0.5 mL). The mixture was then stirred at 25° C. for 2 hrs. On completion, the reaction mixture was concentrated in vacuo to give the title compound (80 mg, HCl) as a white solid. LC-MS (ESI⁺) m/z 887.3 (M+H)⁺.

### Synthesis of (R)-tert-butyl methyl (4-(phenylthio)-3-((4-sulfamoyl-2-((trifluoromethyl)sulfonyl)phenyl)amino)butyl)carbamate (Intermediate OW)

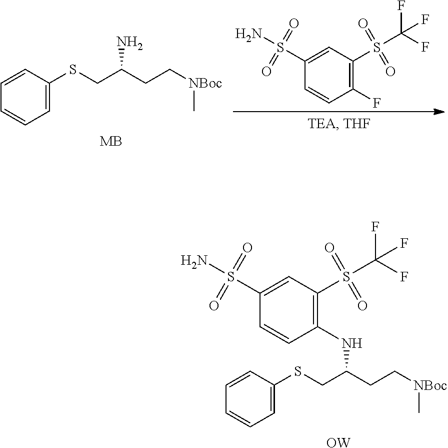

To a solution of (R)-tert-butyl (3-amino-4-(phenylthio)butyl)(methyl)carbamate (400 mg, 1.29 mmol, Intermediate MB) in ACN (2 mL) was added 4-fluoro-3-((trifluoromethyl)sulfonyl)benzenesulfonamide (594 mg, 1.93 mmol, CAS #1027345-08-9) and DIEA (833 mg, 6.44 mmol, 1.12 mL). The reaction was then stirred at 80° C. for 12 hrs. On completion, the reaction mixture was concentrated under reduced pressure to give a residue. The residue was purified reversed-phase HPLC (0.1% FA condition) to give the title compound (350 mg, 41% yield) as a white solid. LC-MS (ESI⁺) m/z 498.2. (M+H)⁺.

### Synthesis of 4-(4-((R)-(4′-chloro-[1,1′-biphenyl]-2-yl)(hydroxy)methyl)piperidin-1-yl)-N-((4-(((R)-4-(methylamino)-1-(phenylthio)butan-2-yl)amino)-3-((trifluoromethyl)sulfonyl)phenyl)sulfonyl)benzamide (Intermediate OX)

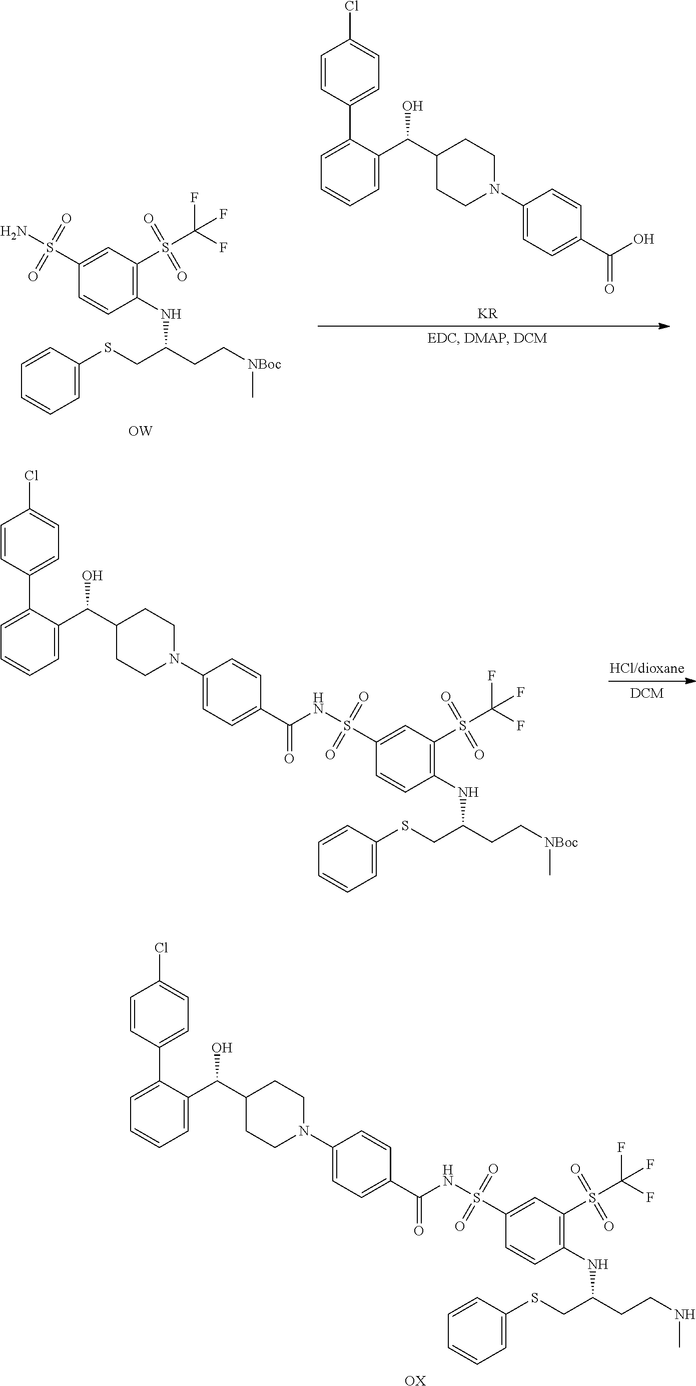

### Step 1—Tert-butyl ((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)carbamate

To a solution of (R)-4-(4-((4′-chloro-[1,1′-biphenyl]-2-yl)(hydroxy)methyl)piperidin-1-yl)benzoic acid (84.7 mg, 201 μmol, Intermediate KR) in DCM (1 mL) was added DMAP (61.3 mg, 502 μmol), EDC (62.3 mg, 402 μmol, 71.1 uL) and (R)-tert-butyl methyl (4-(phenylthio)-3-((4-sulfamoyl-2-((trifluoromethyl)sulfonyl)phenyl)amino)butyl)carbamate (120 mg, 201 μmol, Intermediate OW). The mixture was then stirred at 40° C. for 2 hrs. On completion, the reaction mixture was concentrated under reduced pressure to give a residue. The residue was purified by reversed-phase HPLC (0.1% FA condition) to give the title compound (100 mg, 50% yield) as a yellow solid. LC-MS (ESI⁺) m/z 1001.5. (M+H)⁺.

### Step 2—4-(4-((R)-(4′-chloro-[1,1′-biphenyl]-2-yl)(hydroxy)methyl)piperidin-1-yl)-N-((4-(((R)-4-(methylamino)-1-(phenylthio)butan-2-yl)amino)-3-((trifluoromethyl)sulfonyl)phenyl)sulfonyl)benzamide

To a solution of tert-butyl ((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)carbamate (100 mg, 100 μmol) in DCM (2 mL) was added HCl/dioxane (4 M, 25.0 uL). The reaction was then stirred at 25° C. for 30 mins. On completion, the reaction mixture was concentrated under reduced pressure to give the title compound (95 mg) as a white solid. LC-MS (ESI⁺) m/z 901.4. (M+H)⁺.

### Synthesis of 4-(4-((S)-(4′-chloro-[1,1′-biphenyl]-2-yl)(hydroxy)methyl)piperidin-1-yl)-N-((4-(((R)-4-(methylamino)-1-(phenylthio)butan-2-yl)amino)-3-((trifluoromethyl)sulfonyl)phenyl)sulfonyl)benzamide (Intermediate OY)

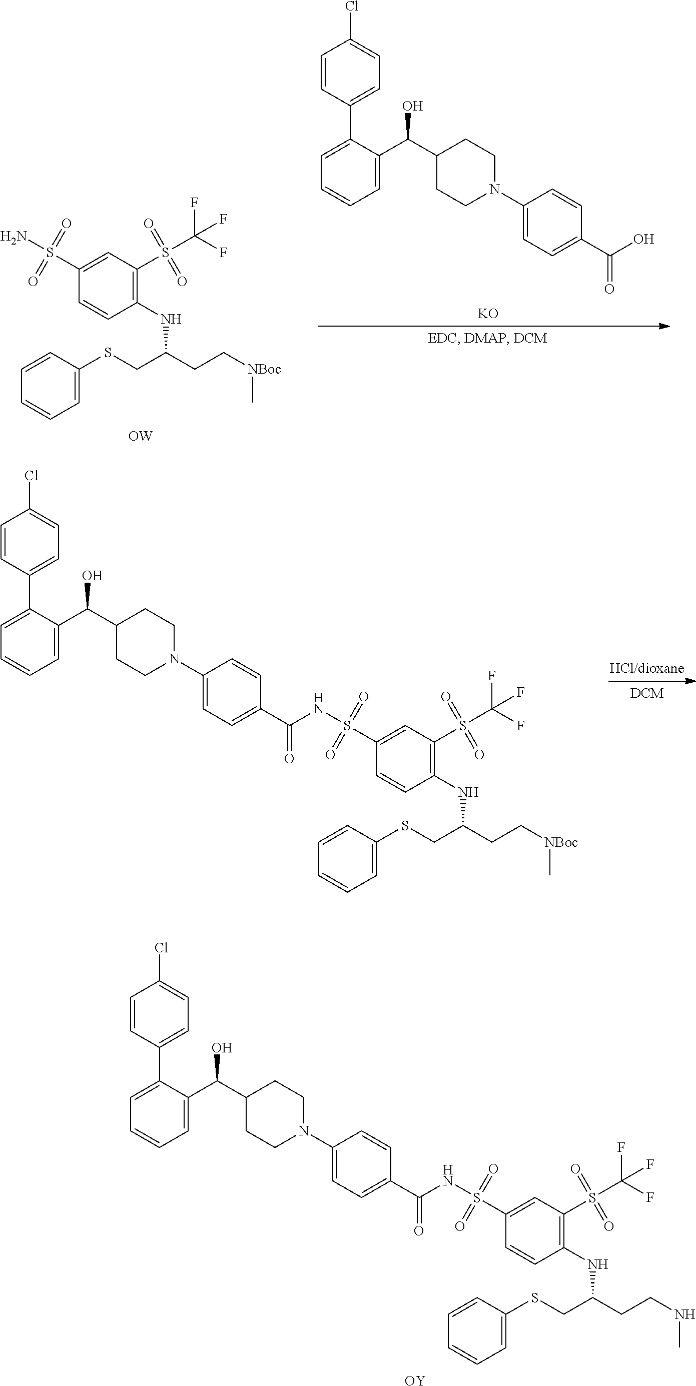

### Step 1—tert-butyl ((R)-3-((4-(N-(4-(4-((S)-(4′-chloro-[1,1′-biphenyl]-2-yl)(hydroxy)methyl)piperidin-1-yl)benzoyl)sulfamoyl)-2-(trifluoromethyl)sulfonyl)phenyl)amino)-4-(phenylthio)butyl)(methyl)carbamate

To a solution of (S)-4-(4-((4′-chloro-[1,1′-biphenyl]-2-yl)(hydroxy)methyl)piperidin-1-yl)benzoic acid (96.2 mg, 228 μmol, Intermediate KO) in DCM (2 mL) was added DMAP (69.6 mg, 570 μmol), EDC (70.8 mg, 456 μmol) and (R)-tert-butyl methyl (4-(phenylthio)-3-((4-sulfamoyl-2-((trifluoromethyl)sulfonyl)phenyl)amino)butyl)carbamate (150 mg, 250 μmol, Intermediate OW). The mixture was then stirred at 40° C. for 1 hr. On completion, the reaction mixture was concentrated under reduced pressure to give the crude product. The crude product was purified by reversed-phase HPLC (0.1% FA condition) to give the title compound (110 mg, 46% yield, FA) as a white solid. LC-MS (ESI+) m/z 1001.5 (M+H)⁺.

### Step 2—4-(4-((S)-(4′-chloro-[1,1′-biphenyl]-2-yl)(hydroxy)methyl)piperidin-1-yl)-N-((4-(((R)-4-(methylamino)-1-(phenylthio)butan-2-yl)amino)-3-((trifluoromethyl)sulfonyl)phenyl)sulfonyl)benzamide

To a solution of tert-butyl ((R)-3-((4-(N-(4-(4-((S)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)carbamate (110 mg, 110 μmol) in DCM (2 mL) was added HCl/dioxane (4 M, 1 mL). The reaction was then stirred at 25° C. for 2 hrs. On completion, the reaction mixture was concentrated under reduced pressure to give the title compound (100 mg) as a white solid. LC-MS (ESI⁺) m/z 901.2. (M+H)⁺.

### Synthesis of 2-((1H-pyrrolo[2,3-b]pyridin-5-yl)oxy)-4-(4-((4′-chloro-[1,1′-biphenyl]-2-yl)methyl)piperazin-1-yl)-N-((4-((3-(methylamino)propyl)amino)-3-((trifluoromethyl)sulfonyl)phenyl)sulfonyl)benzamide (Intermediate OZ)

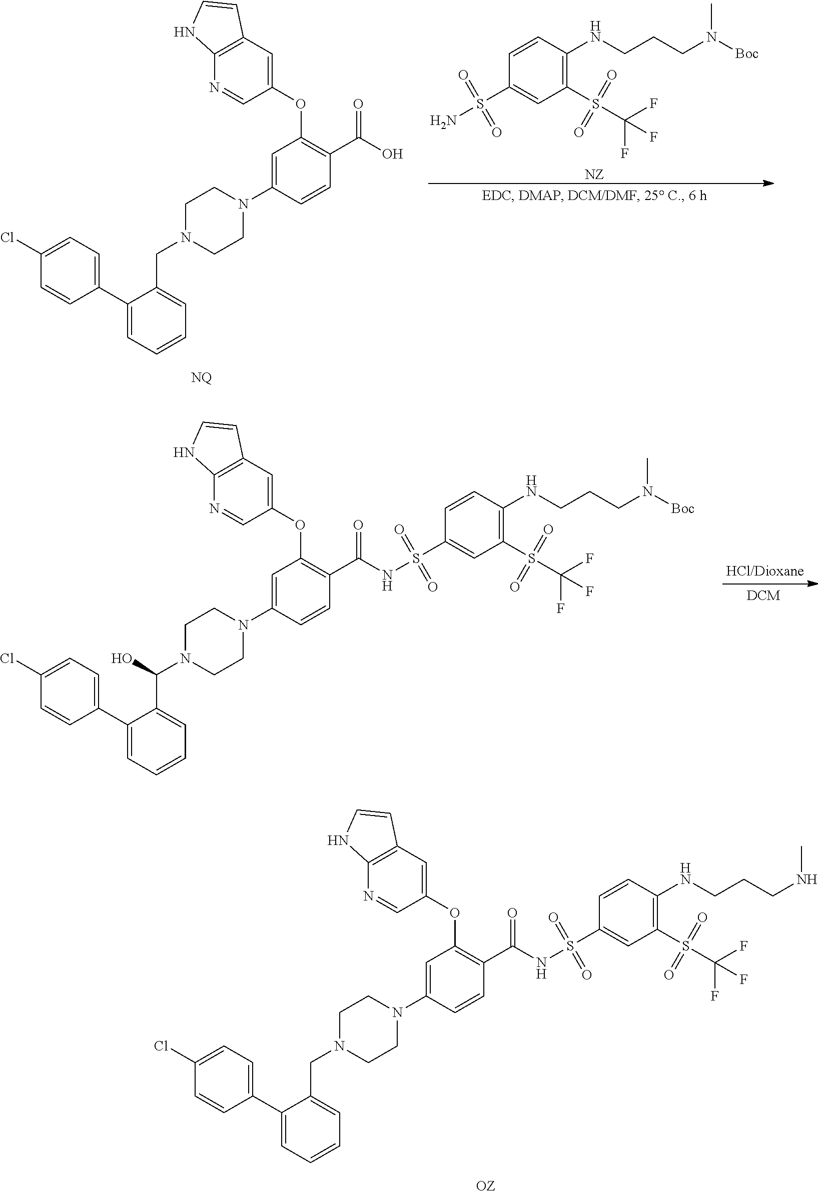

### Step 1—Tert-butyl (3-((4-(N-(2-((1H-pyrrolo[2,3-b]pyridin-5-yl)oxy)-4-(4-((4′-chloro-[1,1′-biphenyl]-2-yl)methyl)piperazin-1-yl)benzoyl)sulfamoyl)-2-((trifluoromethyl)sulfonyl)phenyl)amino)propyl)(methyl)carbamate

To a solution of 2-((1H-pyrrolo[2,3-b]pyridin-5-yl)oxy)-4-(4-((4′-chloro-[1,1′-biphenyl]-2-yl)methyl)piperazin-1-yl)benzoic acid (100 mg, 185 μmol, Intermediate NQ) in DCM (5 mL) and DMF (2 mL) was added EDC (57.6 mg, 371 μmol, 65.6 uL) Then the mixture was stirred for 30 min. Next, tert-butyl methyl (3-((4-sulfamoyl-2-((trifluoromethyl)sulfonyl)phenyl)amino)propyl)carbamate (88.2 mg, 185 μmol, Intermediate NZ) and DMAP (56.6 mg, 463 μmol) was added and the mixture was stirred at 25° C. for 5.5 hrs. On completion, was reaction mixture was concentrated under reduced pressure to remove solvent and purified by reversed-phase HPLC (0.1% FA condition) to give the title compound (100 mg, 52% yield, FA) as a white solid. LC-MS (ESI⁺) m/z 996.5 (M+H)⁺.

### Step 2—2-((1H-pyrrolo[2,3-b]pyridin-5-yl)oxy)-4-(4-((4′-chloro-[1,1′-biphenyl]-2-yl)methyl)piperazin-1-yl)-N-((4-((3-(methylamino)propyl)amino)-3-((trifluoromethyl)sulfonyl)phenyl)sulfonyl)benzamide

To a solution of tert-butyl (3-((4-(N-(2-((1H-pyrrolo[2,3-b]pyridin-5-yl)oxy)-4-(4-((4′-chloro-[1,1′-biphenyl]-2-yl)methyl)piperazin-1-yl)benzoyl)sulfamoyl)-2-((trifluoromethyl)sulfonyl)phenyl)amino)propyl)(methyl)carbamate (100 mg, 95.9 μmol, FA) in DCM (10 mL) was added HCl/dioxane (4 M, 1 mL). The mixture was then stirred at 25° C. for 3 hrs. On completion, the reaction mixture was concentrated under reduced pressure to give the title compound (100 mg, HCl) as white solid. LC-MS (ESI⁺) m/z 896.4 (M+H)⁺.

### Synthesis of 2-((1H-pyrrolo[2,3-b]pyridin-5-yl)oxy)-N-((4-((3-aminopropyl)amino)-3-((trifluoromethyl)sulfonyl)phenyl)sulfonyl)-4-(4-((4′-chloro-[1,1′-biphenyl]-2-yl)methyl)piperazin-1-yl)benzamide (Intermediate PA)

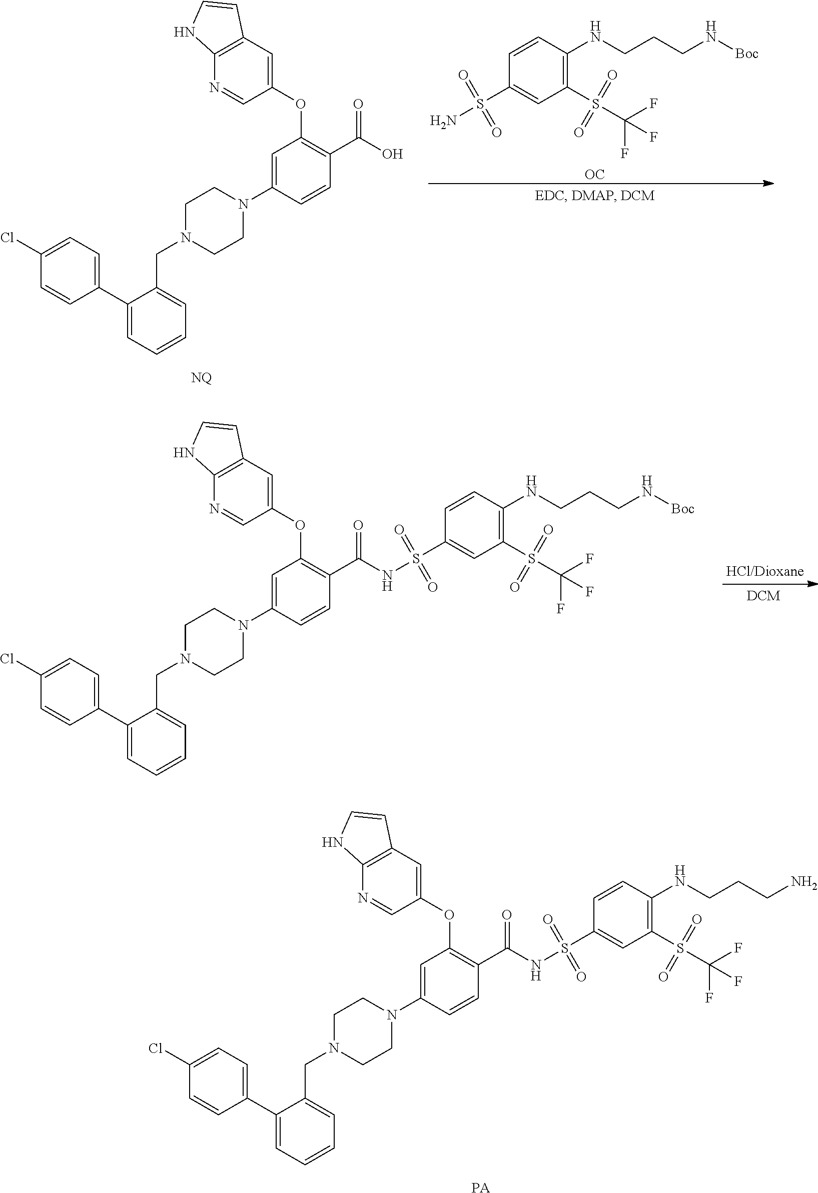

### Step 1—Tert-butyl (3-((4-(N-(2-((1H-pyrrolo[2,3-b]pyridin-5-yl)oxy)-4-(4-((4′-chloro-[1,1′-biphenyl]-2-yl)methyl)piperazin-1-yl)benzoyl)sulfamoyl)-2-((trifluoromethyl)sulfonyl)phenyl)amino)propyl)carbamate

To a solution of 4-[4-[[2-(4-chlorophenyl)phenyl]methyl]piperazin-1-yl]-2-(1H-pyrrolo[2,3-b]pyridin-5-yloxy)benzoic acid (100 mg, 185 μmol, Intermediate NQ) in DCM (5 mL) and DMF (2 mL) was added EDC (57.6 mg, 371 μmol). Then the mixture was stirred for 30 min and tert-butyl N-[3-[4-sulfamoyl-2-(trifluoromethylsulfonyl)anilino]propyl]carbamate (85.6 mg, 185 μmol, Intermediate OC) and DMAP (56.6 mg, 463 μmol) was added. The mixture was then stirred at 25° C. for 5.5 hrs. On completion, the reaction mixture was concentrated under reduced pressure to remove solvent and purified by reversed-phase HPLC (0.1% FA condition) to give the title compound (100 mg, 52% yield, FA) as a white solid. LC-MS (ESI⁺) m/z 982.2 (M+H)⁺.

### Step 2—2-((1H-pyrrolo[2,3-b]pyridin-5-yl)oxy)-N-((4-((3-aminopropyl)amino)-3-((trifluoromethyl)sulfonyl)phenyl)sulfonyl)-4-(4-((4′-chloro-[1,1′-biphenyl]-2-yl)methyl)piperazin-1-yl)benzamide

To a solution of tert-butyl N-[3-[4-[[4-[4-[[2-(4-chlorophenyl)phenyl]methyl]piperazin-1-yl]-2-(1H-pyrrolo[2,3-b]pyridin-5-yloxy)benzoyl]sulfamoyl]-2-(trifluoromethylsulfonyl)anilino]propyl]carbamate (100 mg, 97.2 μmol, FA) in DCM (10 mL) was added HCl/dioxane (4 M, 1 mL). The mixture was then stirred at 25° C. for 1 hr. On completion, the reaction mixture was concentrated under reduced pressure to give the title compound (100 mg, HCl) as white solid. LC-MS (ESI⁺) m/z 882.2 (M+H)⁺.

### Synthesis of (S)-4-(2-(2-(2-cyclopropylphenyl)pyrrolidin-1-yl)-7-azaspiro[3.5]nonan-7-yl)benzoic Acid (Intermediate PB)

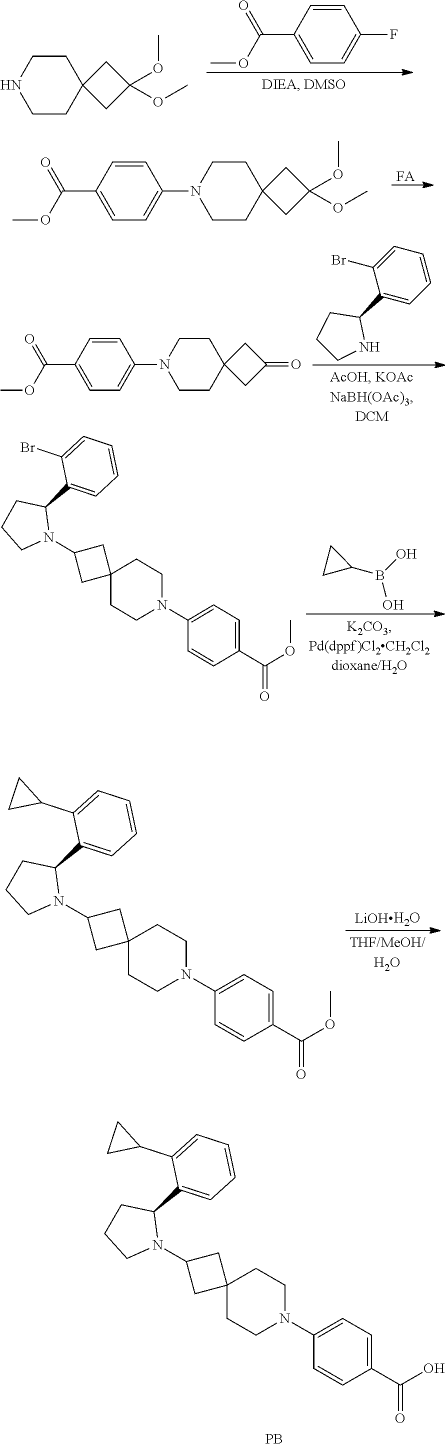

### Step 1—Methyl 4-(2,2-dimethoxy-7-azaspiro[3.5]nonan-7-yl)benzoate

To a solution of methyl 4-fluorobenzoate (1.87 g, 12.1 mmol, CAS #403-33-8) in DMSO (30 mL) was added DIEA (15.7 g, 121 mmol, 21.1 mL) and 2,2-dimethoxy-7-azaspiro[3.5]nonane (1.5 g, 8.10 mmol, CAS #1638766-92-3). The mixture was then stirred at 120° C. for 12 hrs. On completion, the reaction mixture was concentrated under reduced pressure to give a residue. The residue was purified by column chromatography (SiO₂, Petroleum ether/Ethyl acetate=1/0 to 1/1) to give the title compound (700 mg, 25% yield) as a white solid. LC-MS (ESI+) m/z 491.3 (M+H)⁺.

### Step 2—Methyl 4-(2-oxo-7-azaspiro[3.5]nonan-7-yl)benzoate

To a solution of methyl 4-(2,2-dimethoxy-7-azaspiro[3.5]nonan-7-yl)benzoate (630 mg, 1.97 mmol) in DCM (6 mL) was added FA (1.97 mmol). The mixture was stirred at 40° C. for 2 hrs. On completion, the reaction mixture was concentrated under reduced pressure to give a residue. The residue was purified by prep-HPLC (HCl condition) to give the title compound (530 mg) as a white solid. LC-MS (ESI+) m/z 274.2 (M+H)⁺.

### Step 3—Methyl(S)-4-(2-(2-(2-bromophenyl)pyrrolidin-1-yl)-7-azaspiro[3.5]nonan-7-yl)benzoate

To a solution of (S)-2-(2-bromophenyl)pyrrolidine (526 mg, 2.33 mmol, from CAS #1217680-27-7) in DMSO (10 mL) was added AcOH (349 mg, 5.82 mmol, 332 uL), KOAc (285 mg, 2.91 mmol) and methyl 4-(2-oxo-7-azaspiro[3.5]nonan-7-yl)benzoate (530 mg, 1.94 mmol) at 25° C. for 1 hr. Then NaBH(OAc)₃ (1.03 g, 4.85 mmol) was added at 0° C. The mixture was then stirred at 25° C. for 1 hr. On completion, the reaction mixture was concentrated under reduced pressure to give the title compound (940 mg) as a yellow solid. LC-MS (ESI+) m/z 485.0 (M+H)⁺.

### Step 4—Methyl(S)-4-(2-(2-(2-cyclopropylphenyl)pyrrolidin-1-yl)-7-azaspiro[3.5]nonan-7-yl)benzoate

To a solution of cyclopropylboronic acid (177 mg, 2.07 mmol, from CAS #411235-57-9), methyl 4-[2-[(2S)-2-(2-bromophenyl)pyrrolidin-1-yl]-7-azaspiro[3.5]nonan-7-yl]benzoate (200 mg, 413 μmol), K₂CO₃ (371 mg, 2.69 mmol), and Pd(dppf)Cl₂·CH₂Cl₂ (33.7 mg, 41.3 μmol) in dioxane (2.7 mL) and H₂O (0.3 mL) was degassed and purged with N₂ three times. Then the mixture was stirred at 100° C. for 2 hrs under N₂ atmosphere. On completion, the reaction mixture was concentrated under reduced pressure to give the title compound (100 mg) as a black oil. LC-MS (ESI+) m/z 445.1 (M+H)⁺.

### Step 5—(S)-4-(2-(2-(2-cyclopropylphenyl)pyrrolidin-1-yl)-7-azaspiro[3.5]nonan-7-yl)benzoic Acid

To a solution of methyl(S)-4-(2-(2-(2-cyclopropylphenyl)pyrrolidin-1-yl)-7-azaspiro[3.5]nonan-7-yl)benzoate (100 mg, 224 μmol) in THF (2 mL), MeOH (0.5 mL) and H₂O (0.5 mL) was added LiOH·H₂O (28 mg, 674 μmol). The mixture was then stirred at 40° C. for 12 hrs. On completion, the reaction mixture was concentrated under reduced pressure to give the title compound (90 mg) as a white solid. LC-MS (ESI+) m/z 431.2 (M+H)⁺.

### Synthesis of 4-(2-((S)-2-(2-cyclopropylphenyl)pyrrolidin-1-yl)-7-azaspiro[3.5]nonan-7-yl)-N-((4-((R)-1-(phenylthio)-4-(piperazin-1-yl)butan-2-yl)amino)-3-((trifluoromethyl)sulfonyl)phenyl)sulfonyl)benzamide (Intermediate PC)

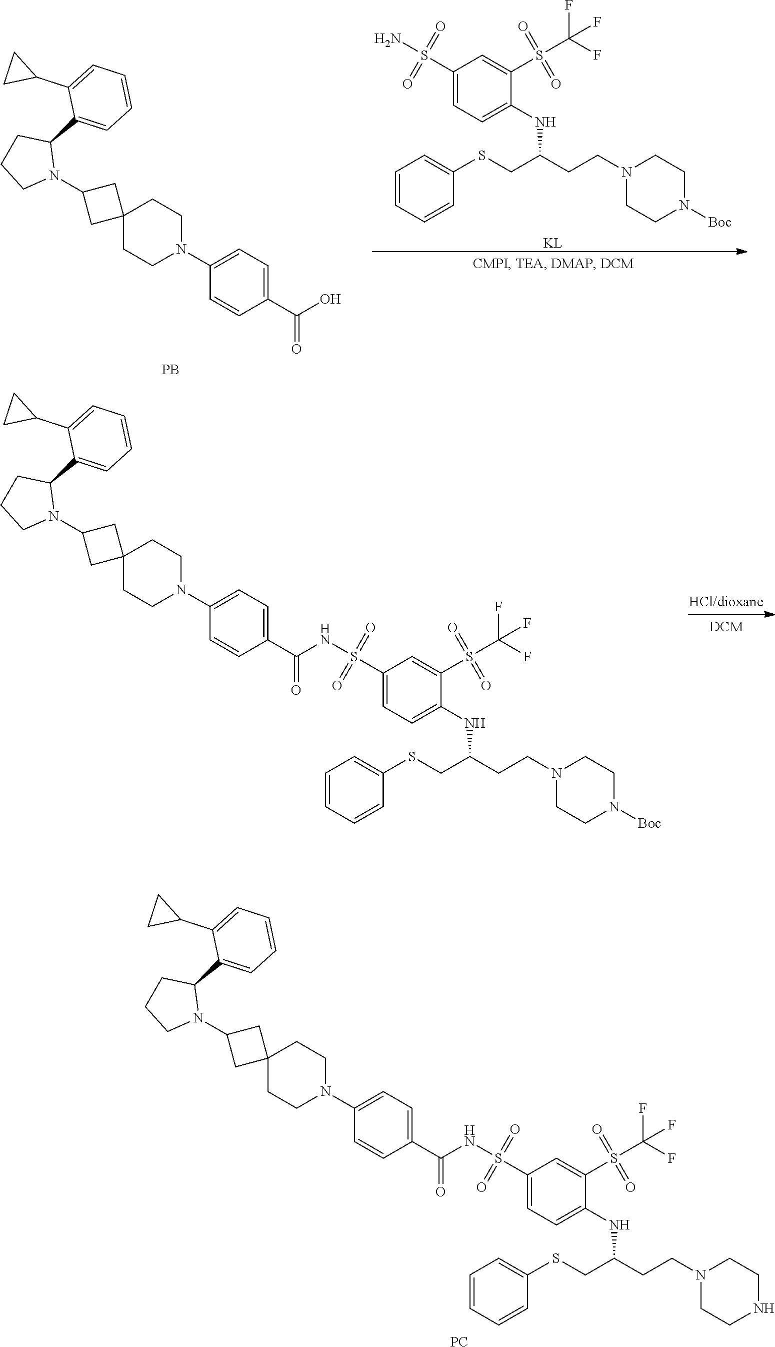

### Step 1—Tert-butyl 4-((R)-3-((4-(N-(4-(2-((S)-2-(2-cyclopropylphenyl)pyrrolidin-1-yl)-7-azaspiro[3.5]nonan-7-yl)benzoyl)sulfamoyl)-2-((trifluoromethyl)sulfonyl)phenyl)amino)-4-(phenylthio)butyl)piperazine-1-carboxylate

To a solution of 4-[2-[(2S)-2-(2-cyclopropylphenyl)pyrrolidin-1-yl]-7-azaspiro[3.5]nonan-7-yl]benzoic acid (86 mg, 199 μmol, Intermediate PB) in DCM (1 mL) was added DMAP (22 mg, 181 μmol), CMPI (69 mg, 272 μmol) TEA (55 mg, 544 μmol, 75 uL) and tert-butyl 4-[(3R)-4-phenylsulfanyl-3-[4-sulfamoyl-2-(trifluoromethylsulfonyl)anilino]butyl]piperazine-1-carboxylate (118 mg, 181 μmol, Intermediate KL). The mixture was then stirred at 25° C. for 2 hrs. On completion, the reaction mixture was concentrated under reduced pressure to give a residue. The residue was purified by prep-HPLC (neutral condition) to give the title compound (80 mg, 35% yield) as a white solid. LC-MS (ESI+) m/z 1065.4 (M+H)⁺.

### Step 2—4-(2-((S)-2-(2-cyclopropylphenyl)pyrrolidin-1-yl)-7-azaspiro[3.5]nonan-7-yl)-N-((4-(((R)-1-(phenylthio)-4-(piperazin-1-yl)butan-2-yl)amino)-3-((trifluoromethyl)sulfonyl)phenyl)sulfonyl)benzamide

To a solution of tert-butyl 4-((R)-3-((4-(N-(4-(2-((S)-2-(2-cyclopropylphenyl)pyrrolidin-1-yl)-7-azaspiro[3.5]nonan-7-yl)benzoyl)sulfamoyl)-2-((trifluoromethyl)sulfonyl)phenyl)amino)-4-(phenylthio)butyl)piperazine-1-carboxylate (70 mg, 65 μmol) in DCM (1 mL) was added HCl/dioxane (4 M, 16.4 uL). The mixture was then stirred at 25° C. for 2 hrs. On completion, the reaction mixture was concentrated under reduced pressure to give the title compound (60 mg, HCl) as a white solid. LC-MS (ESI+) m/z 965.4 (M+H)⁺.

### Synthesis of (S)-2-(2-(2-isopropylphenyl)pyrrolidin-1-yl)-7-azaspiro[3.5]nonane (Intermediate PD)

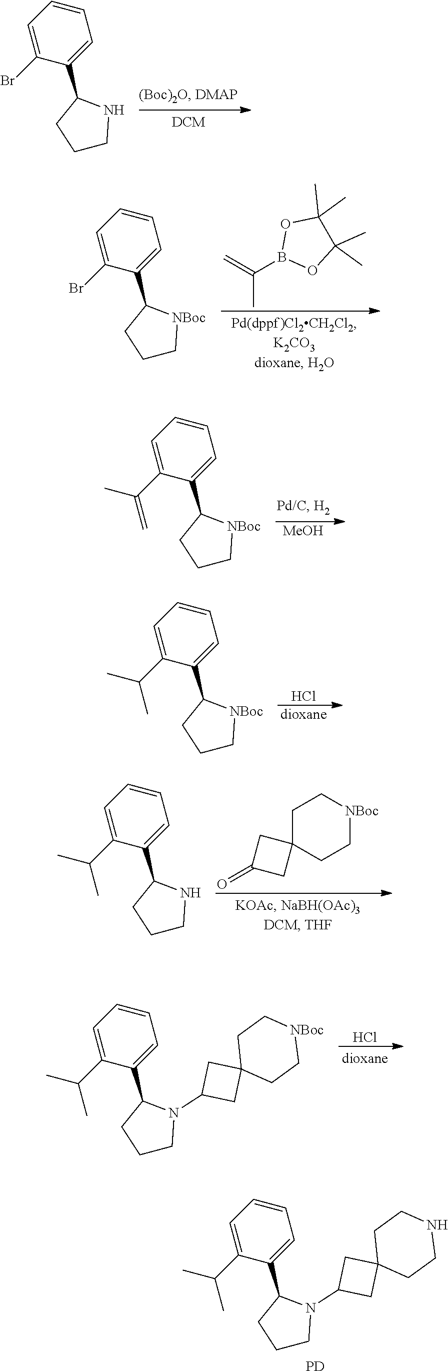

### Step 1—Tert-butyl (S)-2-(2-bromophenyl)pyrrolidine-1-carboxylate

To a solution of (2S)-2-(2-bromophenyl)pyrrolidine (2.00 g, 8.85 mmol, CAS #1217680-27-7) in anhydrous DCM (20.0 mL) was added (Boc)₂O (193 mg, 885 μmol) and DMAP (1.30 g, 10.6 mmol) at 0° C. under nitrogen atmosphere, then the reaction was stirred at 25° C. for 2 hrs under nitrogen atmosphere. On completion, the mixture was concentrated under reduced pressure and purified by column chromatography (SiO₂, Petroleum ether:Ethyl acetate=10/1 to 7/1) to give the title compound (2.10 g, 69% yield) as a white solid. LC-MS (ESI⁺) m/z 349.8 (M+Na)⁺. ¹H NMR (400 MHz, CDCl₃) δ=7.44 (dd, J=8.00, 1.13 Hz, 1H), 7.15-7.23 (m, 1H), 7.04-7.10 (m, 1H), 6.98-7.03 (m, 1H), 4.99-5.21 (m, 1H), 3.52-3.67 (m, 2H), 2.24-2.40 (m, 1H), 1.71-1.87 (m, 3H), 1.11 (s, 9H).

### Step 2—Tert-butyl (S)-2-(2-(prop-1-en-2-yl)phenyl)pyrrolidine-1-carboxylate

A mixture of 2-isopropenyl-4,4,5,5-tetramethyl-1,3,2-dioxaborolane (2.47 g, 14.7 mmol), tert-butyl (2S)-2-(2-bromophenyl)pyrrolidine-1-carboxylate (2.00 g, 6.13 mmol, CAS #126726-62-3), Pd(dppf)Cl₂ (501 mg, 686 μmol), and K₂CO₃ (2.54 g, 18.4 mmol) in dioxane (32 mL) and H₂O (8 mL) was degassed and purged with N₂ three times. Then the mixture was stirred at 80° C. for 16 hrs under N₂ atmosphere. On completion, the reaction mixture was extracted with ethyl acetate (4×10 mL). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether/Ethyl acetate=1/50 to 20/1) to give the title compound (1.75 g, 5.78 mmol, 94.4% yield,) as a white solid. ¹H NMR (400 MHz, CDCl₃) δ=7.19 (d, J=1.75 Hz, 1H), 7.17-7.25 (m, 1H), 7.13-7.17 (m, 1H), 7.13-7.16 (m, 1H), 5.24 (q, J=1.66 Hz, 1H), 4.93-5.02 (m, 1H), 4.82-4.89 (m, 1H), 3.57-3.76 (m, 2H), 2.25-2.38 (m, 1H), 1.92-2.00 (m, 1H), 1.72-1.89 (m, 2H), 1.59 (d, J=4.75 Hz, 2H), 1.01-1.39 (m, 9H).

### Step 3—(4-Bromo-4′-chloro-[1,1′-biphenyl]-2-yl)(piperidin-4-yl) methanol

To a solution of tert-butyl (2S)-2-(2-isopropenylphenyl)pyrrolidine-1-carboxylate (1.75 g, 5.78 mmol) in anhydrous methanol (8.0 mL) and water (8.0 mL) was added Pd/C (61.5 mg, 0.578 mmol) at 25° C. under nitrogen atmosphere, the reaction was then stirred at 25° C. for 5 hrs under nitrogen atmosphere. On completion, the reaction was filtered through kieselguhr very carefully. The filtrate was then concentrated in vacuo to give the title compound (1.75 g).

### Step 4—(S)-2-(2-isopropylphenyl)pyrrolidine

To a solution of tert-butyl (2S)-2-(2-isopropylphenyl)pyrrolidine-1-carboxylate (1.75 g, 6.05 mmol) in anhydrous DCM (20 mL) was added HCl/dioxane (4 M, 15.12 mL) at 25° C. under nitrogen atmosphere, then the reaction was stirred at 25° C. for 1 hr under nitrogen atmosphere. On completion, the reaction mixture was concentrated in vacuo to give the title compound (1.1 g) as a white solid. LC-MS (ESI⁺) m/z 190.3 (M+H)⁺.

### Step 5—Tert-butyl (S)-2-(2-(2-isopropylphenyl)pyrrolidin-1-yl)-7-azaspiro[3.5]nonane-7-carboxylate

To a solution of tert-butyl 2-oxo-7-azaspiro[3.5]nonane-7-carboxylate (1.53 g, 6.39 mmol. CAS #203661-69-2) and (2S)-2-(2-isopropylphenyl)pyrrolidine (1.10 g, 5.81 mmol) in anhydrous DCM (10 mL) and THF (10 mL) was added KOAc (1.14 g, 11.6 mmol) at 25° C. under nitrogen atmosphere. The reaction was stirred at 25° C. for 1 hr under nitrogen atmosphere, then NaBH(OAc)₃ (3.69 g, 17.4 mmol) was added at 25° C., and the mixture was stirred the reaction for another 1 hr. On completion, the reaction mixture was quenched with water (5 mL) and extracted with dichloromethane (3×10 mL). The combined organic layers were washed with brine (5 mL) and dried over anhydrous sodium sulfate, filtered and the filtrate was concentrated in vacuo to give the crude residue. The residue was purified by column chromatography (SiO₂, Dichloromethan:Methanol=30/1 to 20/1) to give the title compound (2.1 g, 84% yield) as a colorless oil. LC-MS (ESI⁺) m/z 413.3 (M+H)⁺. ¹H NMR (400 MHz, CDCl₃) δ=7.22-7.29 (m, 4H), 3.27-3.35 (m, 2H), 3.15 (t, J=5.07 Hz, 3H), 3.09 (d, J=4.88 Hz, 3H), 2.15-2.23 (m, 1H), 2.02-2.09 (m, 1H), 1.70-1.86 (m, 2H), 1.58-1.67 (m, 1H), 1.39-1.50 (m, 6H), 1.33 (s, 9H), 1.30 (s, 2H), 1.25 (d, J=6.63 Hz, 3H), 1.13 (d, J=6.75 Hz, 3H).

### Step 6—(S)-2-(2-(2-isopropylphenyl)pyrrolidin-1-yl)-7-azaspiro[3.5]nonane

To a solution of tert-butyl 2-[(2S)-2-(2-isopropylphenyl)pyrrolidin-1-yl]-7-azaspiro[3.5]nonane-7-carboxylate (2.05 g, 4.97 mmol) in anhydrous DCM (30 mL) was added HCl/dioxane (4 M, 34.3 mL) at 25° C. under nitrogen atmosphere, the reaction was stirred at 25° C. for 2 hrs under nitrogen atmosphere. On completion, the reaction mixture was concentrated in vacuo to give the title compound (2.1 g) as a colorless oil. LC-MS (ESI⁺) m/z 313.3 (M+H)⁺.

### Synthesis of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic Acid (Intermediate PE)

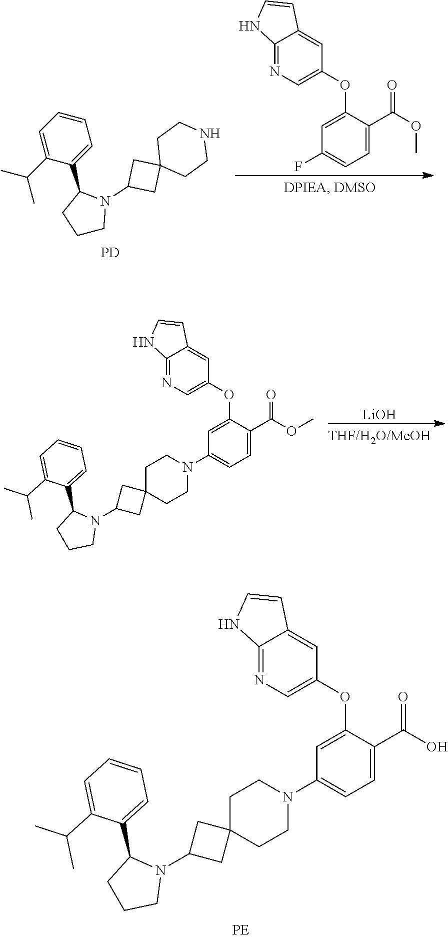

### Step 1—Methyl(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoate

To a solution of 2-[(2S)-2-(2-isopropylphenyl)pyrrolidin-1-yl]-7-azaspiro[3.5]nonane (1.80 g, 5.76 mmol, Intermediate PD) in anhydrous DMSO (10 mL) was added DIPEA (7.44 g, 57.6 mmol, 10.0 mL) and methyl 4-fluoro-2-(1H-pyrrolo[2,3-b]pyridin-5-yloxy)benzoate (4.95 g, 17.3 mmol, CAS #1235865-75-4) at 25° C. under nitrogen atmosphere. Then the reaction was stirred at 120° C. for 48 hrs under nitrogen atmosphere. On completion, the reaction mixture was diluted with water (5 mL) and extracted with dichloromethane (4×10 mL). The combined organic layers were washed with water (3×5 mL) and dried over anhydrous sodium sulfate, filtered and the filtrate was concentrated in vacuo to give the crude residue. The crude residue was purified by reversed-phase HPLC (0.1% ammonium hydroxide) to give the title compound (0.436 g, 43% yield) as a brown solid. LC-MS (ESI⁺) m/z 539.3 (M+H)⁺.

### Step 2—(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic Acid

A mixture of methyl 4-[2-[(2S)-2-(2-isopropylphenyl)pyrrolidin-1-yl]-7-azaspiro[3.5]nonan-7-yl]-2-(1H-pyrrolo[2,3-b]pyridin-5-yloxy)benzoate (0.500 g, 864 μmol), LiOH·H₂O (181 mg, 4.32 mmol) in THF (4 mL), H₂O (2 mL) and MeOH (4 mL), and then the reaction was stirred at 50° C. for 12 hrs under nitrogen atmosphere. On completion, the reaction mixture was added HCl (1 M) until the pH=5, then the mixture was diluted with water (3 mL) and extracted with dichloromethane (5×10 mL). The combined organic layers were dried over anhydrous sodium sulfate, filtered and the filtrate was concentrated in vacuo to give the title compound (0.5 g) as a brown solid. LC-MS (ESI⁺) m/z 565.3 (M+H)⁺. ¹H NMR (400 MHz, CDCl₃) δ=11.61 (s, 1H), 7.98 (d, J=2.50 Hz, 1H), 7.69-7.82 (m, 1H), 7.42-7.60 (m, 2H), 7.38 (d, J=2.13 Hz, 2H), 7.21-7.28 (m, 1H), 7.08-7.18 (m, 1H), 6.68-6.80 (m, 1H), 6.30-6.42 (m, 2H), 3.57-3.69 (m, 3H), 3.11 (s, 3H), 3.02 (s, 2H), 1.93 (s, 1H), 1.70-1.84 (m, 4H), 1.38-1.51 (m, 5H), 1.18 (d, J=9.38 Hz, 6H).

### Synthesis of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((4-((3-(piperazin-1-yl)propyl)amino)-3-((trifluoromethyl)sulfonyl)phenyl)sulfonyl)benzamide (Intermediate PF)

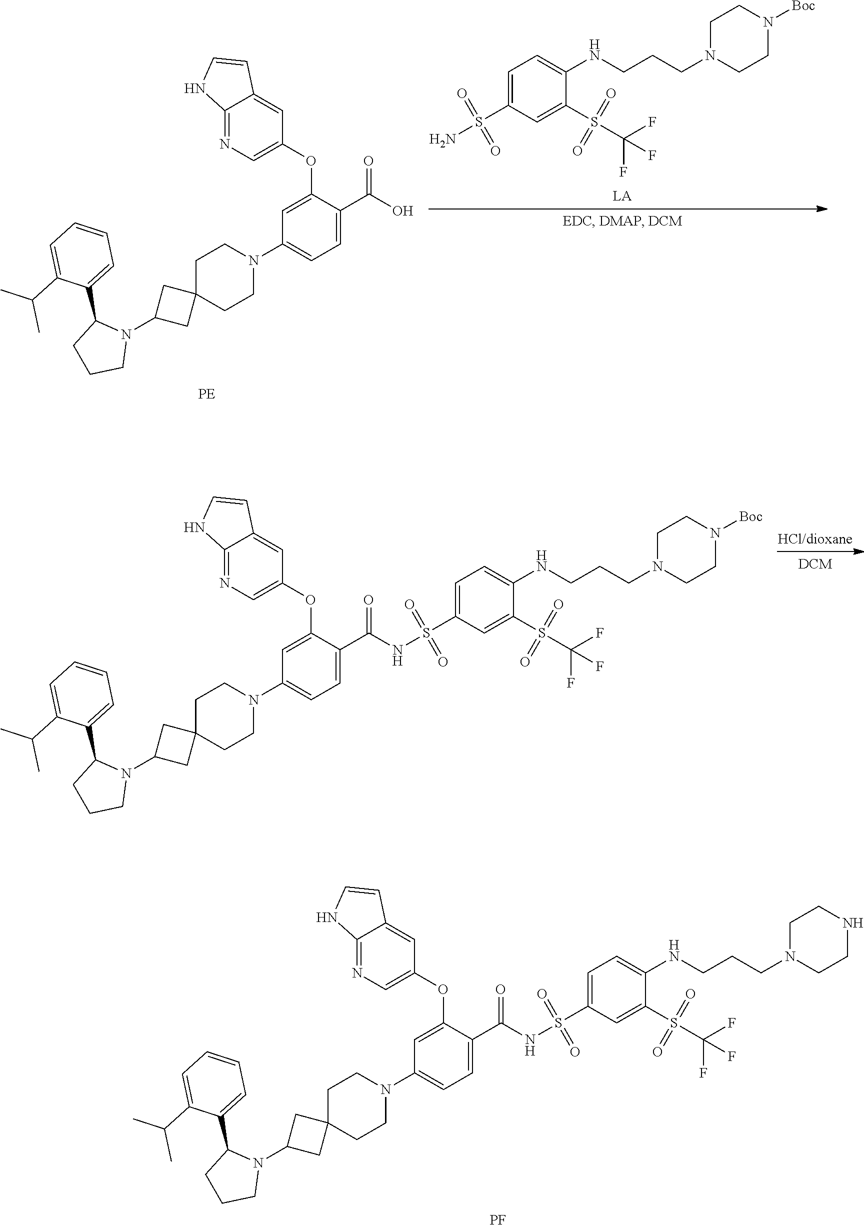 [SMILES: CC(C)c1ccccc1[C@@H]1CCCN1C1CC2(CCN(c3ccc(C(=O)O)c(Oc4cnc5[nH]ccc5c4)c3)CC2)C1 ; CC(C)(C)OC(=O)N1CCN(CCCNc2ccc(S(N)(=O)=O)cc2S(=O)(=O)C(F)(F)F)CC1 ; CC(C)c1ccccc1[C@@H]1CCCN1C1CC2(CCN(c3ccc(C(=O)NS(=O)(=O)c4ccc(NCCCN5CCN(C(=O)OC(C)(C)C)CC5)c(S(=O)(=O)C(F)(F)F)c4)c(Oc4cnc5[nH]ccc5c4)c3)CC2)C1 ; CC(C)c1ccccc1[C@@H]1CCCN1C1CC2(CCN(c3ccc(C(=O)NS(=O)(=O)c4ccc(NCCCN5CCNCC5)c(S(=O)(=O)C(F)(F)F)c4)c(Oc4cnc5[nH]ccc5c4)c3)CC2)C1 ; "PF"]

### Step 1—Tert-butyl (S)-4-(3-((4-(N-(2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoyl)sulfamoyl)-2-((trifluoromethyl)sulfonyl)phenyl)amino)propyl)piperazine-1-carboxylate

To a of tert-butyl 4-[3-[4-sulfamoyl-2-solution (trifluoromethylsulfonyl)anilino]propyl]piperazine-1-carboxylate (470 mg, 885 μmol, Intermediate LA) in anhydrous DCM (6 mL) and 4-[2-[(2S)-2-(2-isopropylphenyl)pyrrolidin-1-yl]-7-azaspiro[3.5]nonan-7-yl]-2-(1H-pyrrolo[2,3-b]pyridin-5-yloxy)benzoic acid (0.5 g, 885 μmol, Intermediate PE) was added DMAP (270 mg, 2.21 mmol) and EDC (274 mg, 1.77 mmol, 313 uL) at 25° C. under nitrogen atmosphere. Then the reaction was stirred at 25° C. for 12 hrs under nitrogen atmosphere. On completion, the reaction mixture was diluted with water (1 mL) and extracted with dichloromethane (3×2 mL). The combined organic layers were washed with brine (2 mL) and dried over anhydrous sodium sulfate, filtered and the filtrate was concentrated in vacuo to give the crude residue. The crude residue was purified by reversed-phase HPLC (0.1% FA condition) to give the title compound (0.436 g, 43% yield) as a brown solid. LC-MS (ESI⁺) m/z 1259.5 (M+H)⁺. ¹H NMR (400 MHz, DMSO-d₆) δ=11.57-11.81 (m, 1H), 8.06-8.11 (m, 1H), 7.98-8.04 (m, 1H), 7.82-7.90 (m, 1H), 7.39-7.59 (m, 4H), 7.10-7.26 (m, 3H), 6.86-6.99 (m, 1H), 6.60-6.70 (m, 1H), 6.37 (s, 1H), 6.15-6.22 (m, 1H), 3.01 (s, 1H), 2.93 (s, 2H), 2.27-2.39 (m, 7H), 1.67-1.75 (m, 3H), 1.36 (s, 3H), 1.10-1.26 (m, 7H).

### Step 2—(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((4-((3-(piperazin-1-yl)propyl)amino)-3-((trifluoromethyl)sulfonyl)phenyl)sulfonyl)benzamide

To a solution of tert-butyl 4-[3-[4-[[4-[2-[(2S)-2-(2-isopropylphenyl)pyrrolidin-1-yl]-7-azaspiro[3.5]nonan-7-yl]-2-(1H-pyrrolo[2,3-b]pyridin-5-yloxy)benzoyl]sulfamoyl]-2-(trifluoromethylsulfonyl)anilino]propyl]piperazine-1-carboxylate (0.3 g, 278.48 μmol) in DCM (8 mL) was added HCl/dioxane (4 M, 3 mL) at 25° C. under nitrogen atmosphere, the reaction was then stirred at 25° C. for 1 hrs under nitrogen atmosphere. On completion, the reaction mixture was concentrated under reduced pressure to give the title compound (0.29 g) as a white solid. LC-MS (ESI⁺) m/z 489.4 (M+H)⁺.

### Synthesis of Tert-butyl 4-(((4-sulfamoyl-2-((trifluoromethyl)sulfonyl)phenyl)amino)methyl) piperidine-1-carboxylate (Intermediate PG)

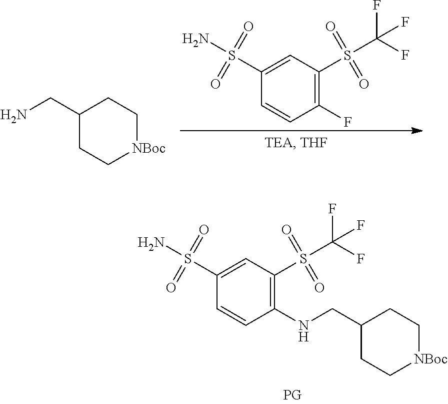 [SMILES: CC(C)(C)OC(=O)N1CCC(CN)CC1 ; NS(=O)(=O)c1ccc(F)c(S(=O)(=O)C(F)(F)F)c1 ; CC(C)(C)OC(=O)N1CCC(CNc2ccc(S(N)(=O)=O)cc2S(=O)(=O)C(F)(F)F)CC1]

To a solution of tert-butyl 4-(aminomethyl)piperidine-1-carboxylate (1 g, 4.67 mmol, CAS #144222-22-0) in THF (15 mL) was added TEA (1.42 g, 14.0 mmol, 1.95 mL) and 4-fluoro-3-((trifluoromethyl)sulfonyl)benzenesulfonamide (1.43 g, 4.67 mmol, CAS #1027345-08-9). The mixture was stirred at 60° C. for 5 hrs. On completion, the reaction mixture was diluted with H₂O (30 mL) and extracted with EA (30 mL×3). The combined organic layers were dried over Na₂SO₄, filtered and concentrated under reduced pressure to give the title compound (2.30 g) as a yellow solid. LC-MS (ESI⁺) m/z 402.5 (M+H-Boc)⁺.

### Synthesis of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((4-((piperidin-4-ylmethyl)amino)-3-((trifluoromethyl)sulfonyl)phenyl) sulfonyl)benzamide (Intermediate PH)

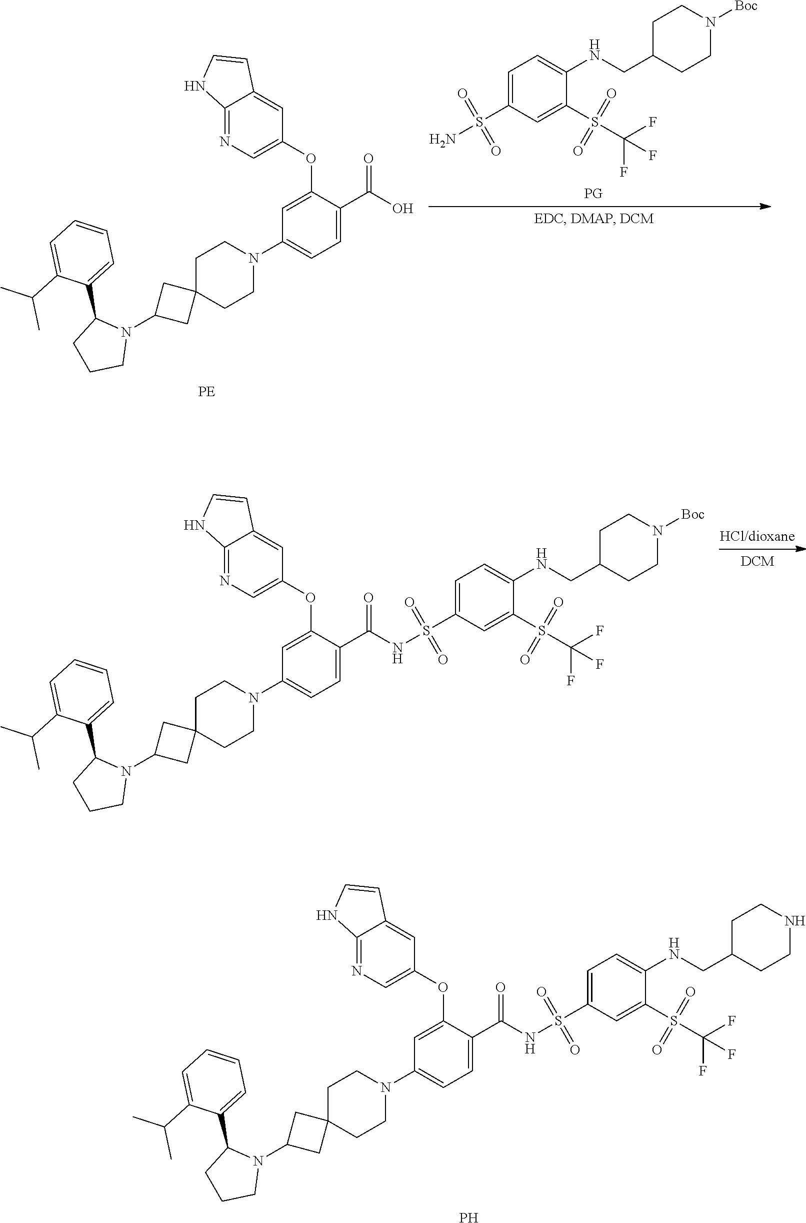

### Step 1—Tert-butyl (S)-4-(((4-(N-(2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoyl)sulfamoyl)-2-((trifluoromethyl)sulfonyl)phenyl)amino)methyl)piperidine-1-carboxylate

To a solution of (S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoic acid (428 mg, 758 μmol, Intermediate PE) in DCM (5 mL) was added EDC (294 mg, 1.89 mmol), and DMAP (231 mg, 1.89 mmol). Then tert-butyl 4-(((4-sulfamoyl-2-((trifluoromethyl)sulfonyl)phenyl)amino)methyl)piperidine-1-carboxylate (380 mg, 758 μmol, Intermediate PG) was added and the mixture was stirred at 25° C. for 12 hrs. On completion, the reaction mixture was concentrated under reduced pressure to give the crude product. The crude product was purified by reversed-phase HPLC (0.1% HCl condition) to give the title compound (300 mg, 30% yield) as a pink solid. LC-MS (ESI⁺) m/z 1048.4 (M+H)⁺.

### Step 2—(S)-2-((1H-pyrrolo[2,3-b]pyridin-5-yl)oxy)-4-(2-(2-(2-isopropylphenyl)pyrrolidin-1-yl)-7-azaspiro[3.5]nonan-7-yl)-N-((4-((piperidin-4-ylmethyl)amino)-3-((trifluoromethyl)sulfonyl)phenyl)sulfonyl)benzamide

To a solution of tert-butyl (S)-4-(((4-(N-(2-((1H-pyrrolo[2,3-b]pyridin-5-yl)oxy)-4-(2-(2-(2-isopropylphenyl)pyrrolidin-1-yl)-7-azaspiro[3.5]nonan-7-yl)benzoyl)sulfamoyl)-2-((trifluoromethyl)sulfonyl)phenyl)amino)methyl)piperidine-1-carboxylate (300 mg, 286 μmol) in DCM (5 mL) was added HCl/dioxane (4 M, 71.5 uL). The mixture was then stirred at 25° C. for 2 hrs. On completion, the reaction mixture was concentrated under reduced pressure to give the title compound (300 mg, HCl) as a gray solid. LC-MS (ESI⁺) m/z 948.5 (M+H)⁺.

### Synthesis of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((piperidin-4-ylmethyl)amino)-3-(trifluoromethyl)sulfonyl)phenyl)sulfonyl)benzamide (Intermediate PI)

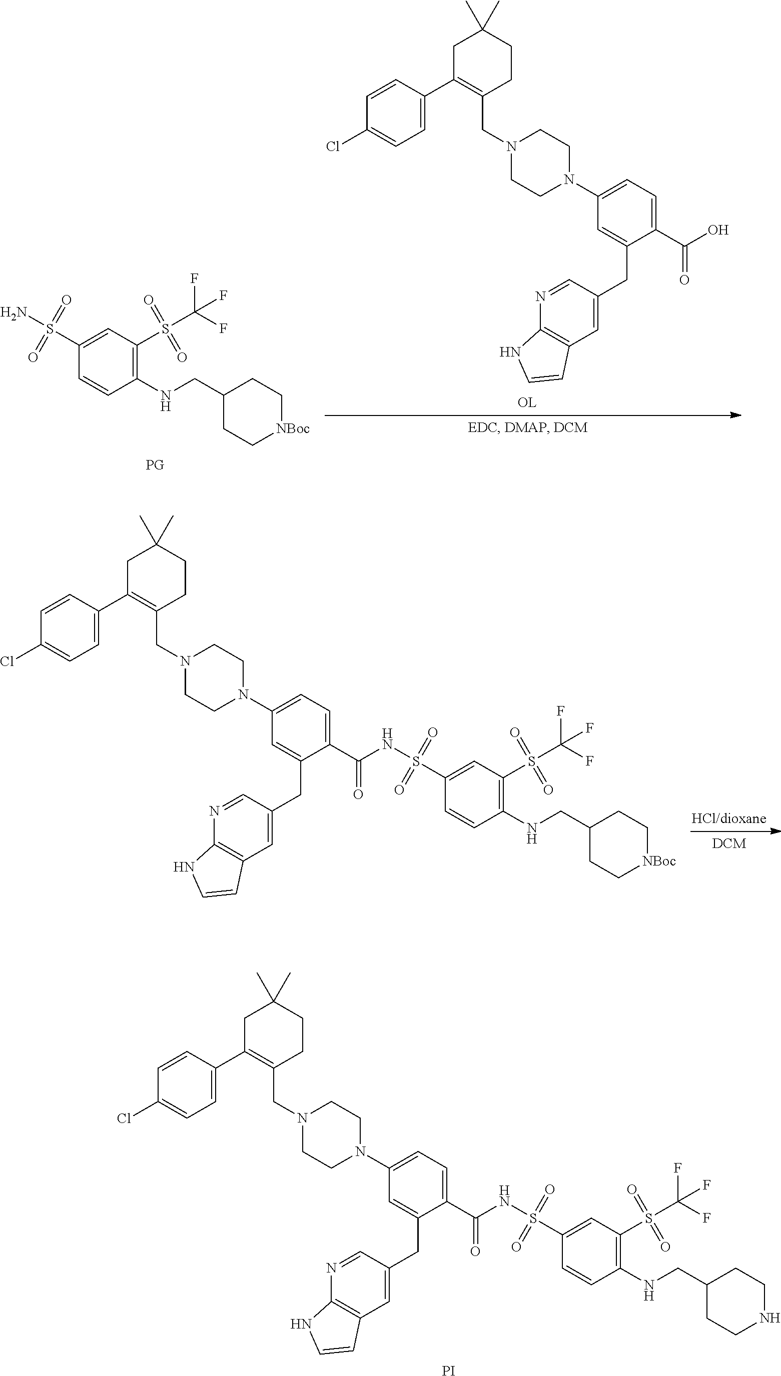 [SMILES: CC(C)(C)OC(=O)N1CCC(CNc2ccc(S(N)(=O)=O)cc2S(=O)(=O)C(F)(F)F)CC1 ; CC1(C)CCC(CN2CCN(c3ccc(C(=O)O)c(Cc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(NCC5CCN(C(=O)OC(C)(C)C)CC5)c(S(=O)(=O)C(F)(F)F)c4)c(Cc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(NCC5CCNCC5)c(S(=O)(=O)C(F)(F)F)c4)c(Cc4cnc5[nH]ccc5c4)c3)CC2)=C(c2ccc(Cl)cc2)C1 ; "PI"]

### Step 1—Tert-butyl 4-(((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)methyl)piperidine-1-carboxylate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (200 mg, 350 μmol, Intermediate OL) in DCM (5 mL) was added EDC (1361 mg, 875 μmol), DMAP (107 mg, 875 μmol), and tert-butyl 4-(((4-sulfamoyl-2-((trifluoromethyl)sulfonyl)phenyl)amino)methyl)piperidine-1-carboxylate (211 mg, 420 μmol, Intermediate PG). The mixture was then stirred at 20° C. for 12 hrs. On completion, the reaction mixture was washed with H₂O (5 mL×3), and the organic layer was concentrated under reduced pressure to give a residue. The residue was purified by prep-HPLC (neutral condition) to give the title compound (170 mg, 45% yield) as a yellow solid. LC-MS (ESI⁺) m/z 1054.4 (M+H)⁺.

### Step 2—2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((piperidin-4-ylmethyl)amino)-3-((trifluoromethyl)sulfonyl)phenyl)sulfonyl)benzamide

To a solution of tert-butyl 4-(((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)methyl)piperidine-1-carboxylate (170 mg, 161 μmol) in DCM (8 mL) was added HCl/dioxane (4 M, 2 mL). The mixture was then stirred at 25° C. for 1 hr. On completion, the reaction mixture was concentrated under reduced pressure to give a residue to give the title compound (230 mg, HCl) as a light yellow solid. LC-MS (ESI⁺) m/z 956.1 (M+H)⁺.

### Synthesis of 2-((1H-pyrrolo[2,3-b]pyridin-5-yl)oxy)-N-((4-((3-aminopropyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide (Intermediate PJ)

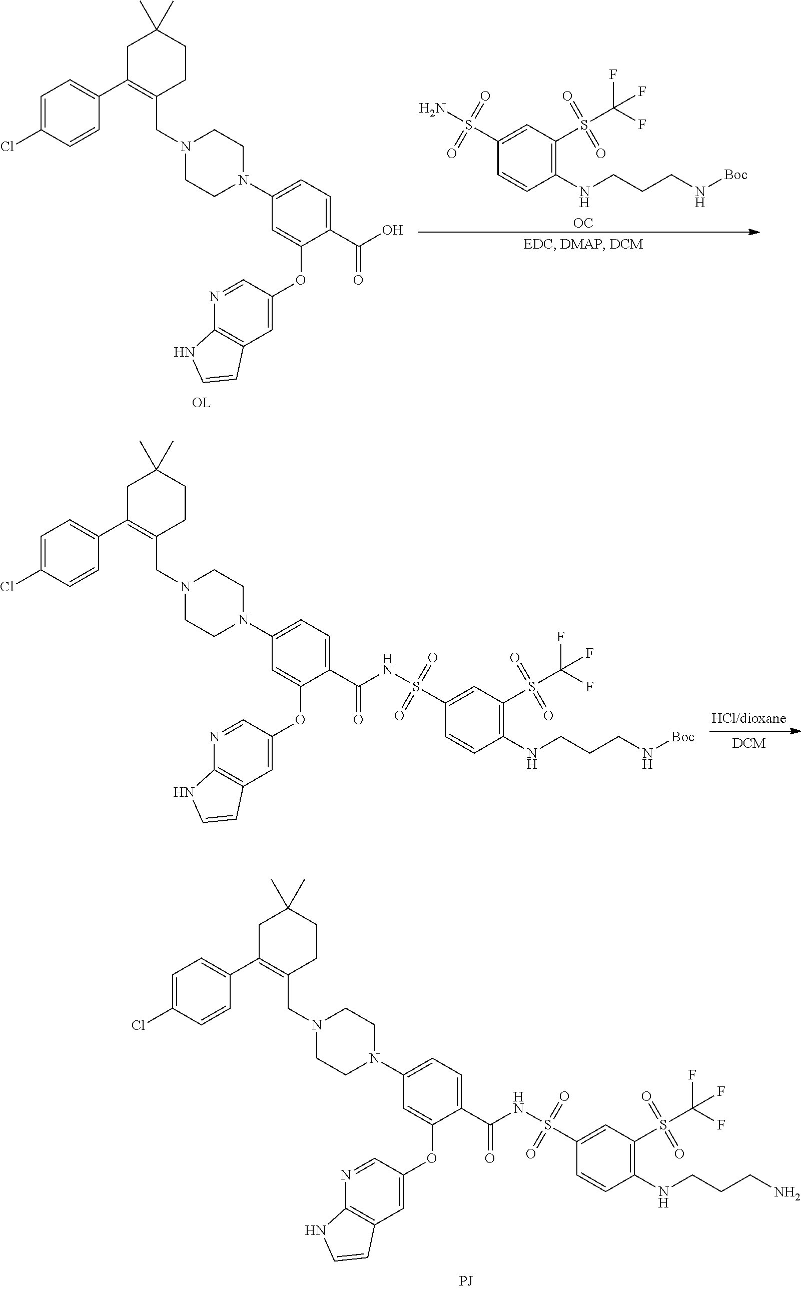

### Step 1—Tert-butyl (3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)carbamate

A solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (300 mg, 525 μmol, Intermediate OL), tert-butyl (3-((4-sulfamoyl-2-((trifluoromethyl)sulfonyl)phenyl)amino)propyl)carbamate (242 mg, 525 μmol, Intermediate OC), EDC (163 mg, 1.05 mmol, 185.97 uL), and DMAP (160 mg, 1.31 mmol) in DCM (3 mL) was stirred at 25° C. for 12 h. On completion, the mixture was concentrated in vacuo to give a residue. The residue was purified by prep-HPLC (0.8 g/L ammonium bicarbonate) to give the title compound (108 mg) as a yellow solid. LC-MS (ESI⁺) m/z 1014.2 (M+H)⁺.

### Step 2—2-((1H-pyrrolo[2,3-b]pyridin-5-yl)oxy)-N-((4-((3-aminopropyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide

A solution of tert-butyl (3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)carbamate (108 mg, 106 μmol) in HCl/dioxane (0.1 mL) and DCM (1 mL) was stirred at 25° C. for 2 h. On completion, the mixture was concentrated in vacuo to give the title compound (100 mg) as a white solid. ¹H NMR (400 MHz, DMSO-d₆) δ=11.81 (s, 1H), 11.72 (s, 1H), 10.71 (d, J=2.0 Hz, 1H), 8.18 (d, J=2.0 Hz, 1H), 8.07 (d, J=2.4 Hz, 1H), 7.62 (d, J=2.4 Hz, 1H), 7.55 (t, J=2.8 Hz, 1H), 7.52-7.44 (m, 2H), 7.39 (d, J=8.0 Hz, 2H), 7.15 (d, J=9.6 Hz, 1H), 7.09 (d, J=8.4 Hz, 2H), 6.72 (d, J=9.4 Hz, 1H), 6.44 (s, 1H), 6.24 (s, 1H), 3.54-3.45 (m, 5H), 3.31-3.22 (m, 4H), 2.84-2.63 (m, 5H), 2.34 (s, 2H), 2.00 (s, 2H), 1.86-1.79 (m, 2H), 1.43 (t, J=6.0 Hz, 2H), 0.93 (s, 6H).

### Synthesis of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-(methylamino)propyl)amino)-3-((trifluoromethyl)sulfonyl)phenyl)sulfonyl)benzamide (Intermediate PK)

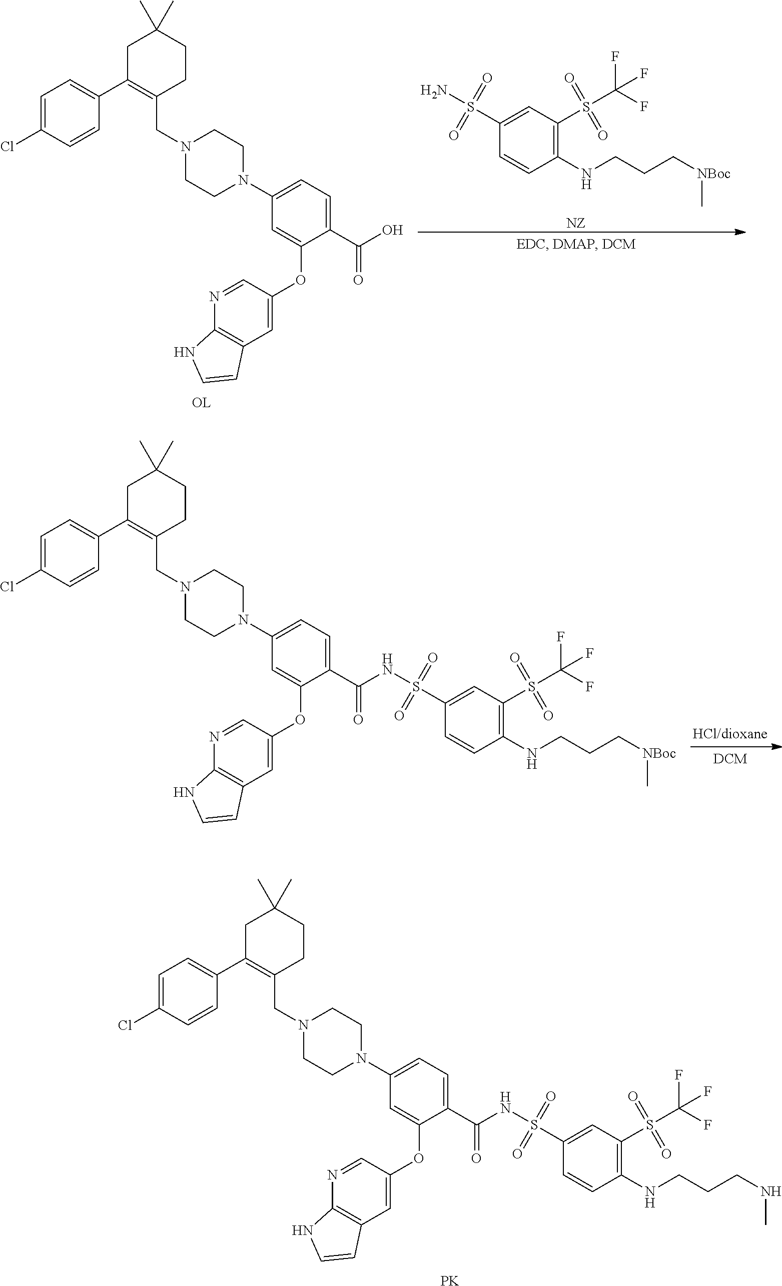 [SMILES: CC1(C)CCC(CN2CCN(c3ccc(C(=O)O)c(Oc4cnc5[nH]ccc5c4)c3)CC2)=C(c2ccc(Cl)cc2)C1 ; CN(CCCNc1ccc(S(N)(=O)=O)cc1S(=O)(=O)C(F)(F)F)C(=O)OC(C)(C)C ; "NZ" ; CN(CCCNc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1S(=O)(=O)C(F)(F)F)C(=O)OC(C)(C)C ; CNCCCNc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1S(=O)(=O)C(F)(F)F ; "PK"]

### Step 1—Tert-butyl (3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)(methyl)carbamate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (300 mg, 525 μmol Intermediate OL) in DCM (4 mL) was added tert-butyl methyl (3-((4-sulfamoyl-2-((trifluoromethyl)sulfonyl)phenyl)amino)propyl)carbamate (300 mg, 631 μmol, Intermediate NZ), EDC (245 mg, 1.58 mmol) and DMAP (193 mg, 1.58 mmol). The mixture was then stirred at 25° C. for 4 hrs. On completion, the reaction mixture was concentrated under reduced pressure to give a crude product. The crude product was purified by reversed-phase HPLC (0.1% FA condition) to give the title compound (200 mg, 25% yield) as a white solid. LC-MS (ESI⁺) m/z 1029.9 (M+2)⁺.

### Step 2 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-(methylamino)propyl)amino)-3-((trifluoromethyl)sulfonyl)phenyl)sulfonyl)benzamide

To a solution of tert-butyl (3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)(methyl)carbamate (180 mg, 175 μmol) in DCM (5 mL) was added HCl/dioxane (4 M, 1 mL). The mixture was then stirred at 25° C. for 2 hrs. On completion, the reaction mixture was concentrated in vacuo to give the title compound (200 mg, HCl) as a white solid. LC-MS (ESI⁺) m/z 929.0 (M+2)⁺.

### Synthesis of Tert-butyl methyl (2-((4-sulfamoyl-2-((trifluoromethyl)sulfonyl)phenyl)amino)ethyl)carbamate (Intermediate PL)

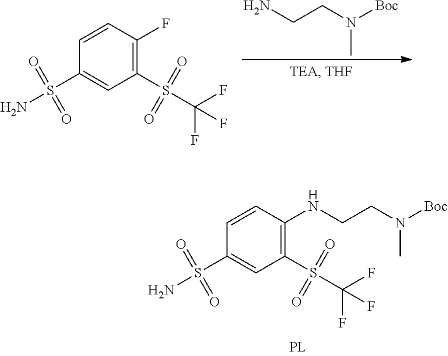 [SMILES: NS(=O)(=O)c1ccc(F)c(S(=O)(=O)C(F)(F)F)c1 ; CN(CCN)C(=O)OC(C)(C)C ; CN(CCNc1ccc(S(N)(=O)=O)cc1S(=O)(=O)C(F)(F)F)C(=O)OC(C)(C)C]

A solution of 4-fluoro-3-((trifluoromethyl)sulfonyl)benzenesulfonamide (882 mg, 2.87 mmol, CAS #1027345-08-9), tert-butyl (2-aminoethyl)(methyl)carbamate (500 mg, 2.87 mmol, 513 uL, CAS #121492-06-6), and TEA (871 mg, 8.61 mmol, 1.20 mL) in THF (9 mL) was stirred at 50° C. for 12 hrs. On completion, the reaction mixture was quenched with water (10 mL) and extracted with ethyl acetate (3×10 mL). The combined organic layers were washed with brine (20 mL) and dried over anhydrous sodium sulfate, filtered and the filtrate was concentrated in vacuo to give the title compound (1.28 g) as a white solid. LC-MS (ESI⁺) m/z 484.1 (M+Na)⁺.

### Synthesis of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((2-(methylamino)ethyl)amino)-3-((trifluoromethyl)sulfonyl)phenyl)sulfonyl)benzamide (Intermediate PM)

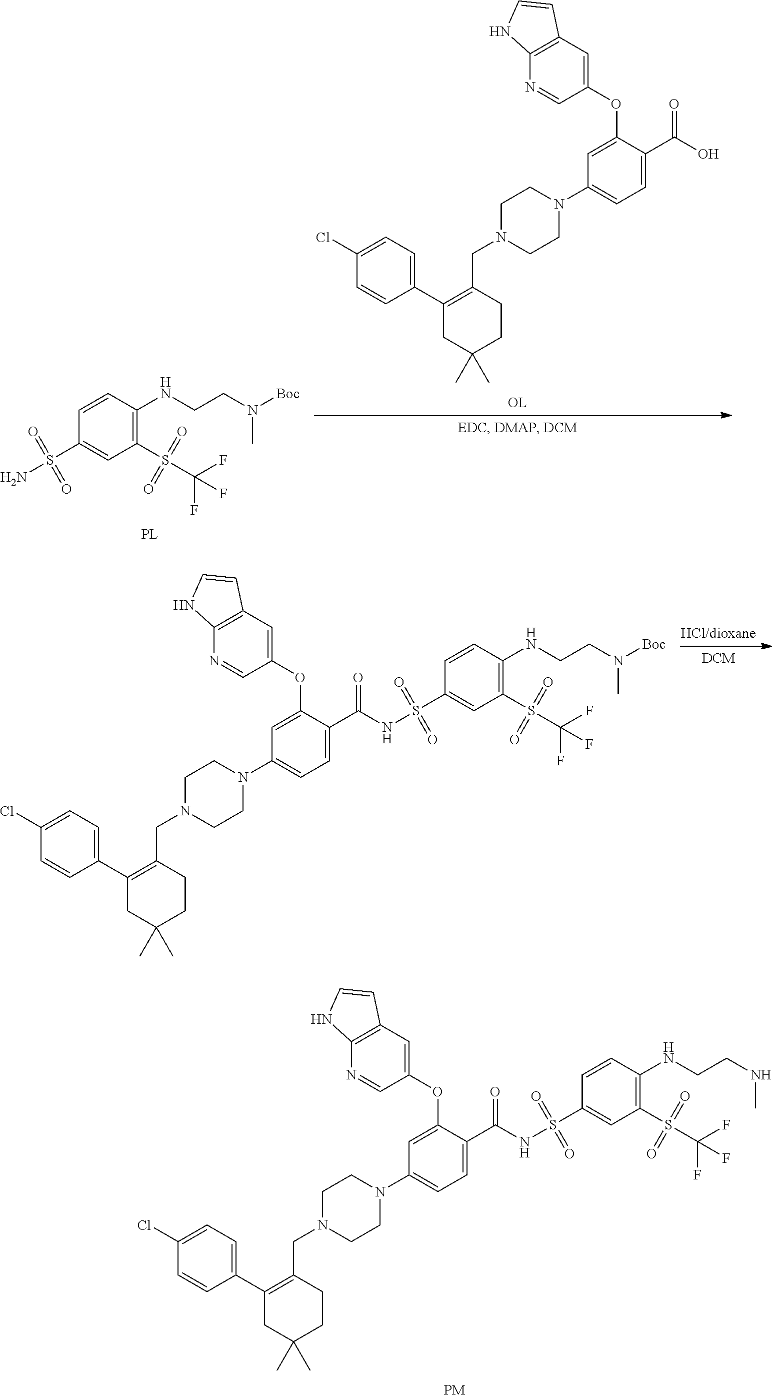 [SMILES: CC1(C)CCC(CN2CCN(c3ccc(C(=O)O)c(Oc4cnc5[nH]ccc5c4)c3)CC2)=C(c2ccc(Cl)cc2)C1 ; CN(CCNc1ccc(S(N)(=O)=O)cc1S(=O)(=O)C(F)(F)F)C(=O)OC(C)(C)C ; CN(CCNc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1S(=O)(=O)C(F)(F)F)C(=O)OC(C)(C)C ; CNCCNc1ccc(S(=O)(=O)NC(=O)c2ccc(N3CCN(CC4=C(c5ccc(Cl)cc5)CC(C)(C)CC4)CC3)cc2Oc2cnc3[nH]ccc3c2)cc1S(=O)(=O)C(F)(F)F]

### Step 1—Tert-butyl (2-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)ethyl)(methyl)carbamate

A solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (371 mg, 650 μmol, Intermediate OL), tert-butyl methyl (2-((4-sulfamoyl-2-((trifluoromethyl)sulfonyl)phenyl)amino)ethyl)carbamate (200 mg, 433 μmol, Intermediate PL), EDC (135 mg, 867 μmol, 153 uL), and DMAP (132 mg, 1.08 mmol) in DCM (2 mL) was stirred at 25° C. for 12 hrs. On completion, the mixture was concentrated in vacuo to give the residue. The crude residue was purified by reversed-phase HPLC (0.8 g/L ammonium bicarbonate) to give the title compound (256 mg) as a light white solid. LC-MS (ESI⁺) m/z 1014.9 (M+H)⁺.

### Step 2—2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((2-(methylamino)ethyl)amino)-3-((trifluoromethyl)sulfonyl)phenyl)sulfonyl)benzamide

A solution of tert-butyl (2-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)ethyl)(methyl)carbamate (250 mg, 246 μmol) in DCM (2 mL) and HCl/dioxane (2 mL) was stirred at 25° C. for 2 hrs. On completion, the mixture was concentrated in vacuo to give the title compound (200 mg, HCl) as a yellow solid. ¹H NMR (400 MHz, DMSO-d₆) δ=11.91 (s, 1H), 11.76 (s, 1H), 10.88 (d, J=2.8 Hz, 1H), 9.17 (s, 2H), 8.19 (d, J=2.0 Hz, 1H), 8.09 (d, J=2.4 Hz, 1H), 8.01-7.92 (m, 1H), 7.67 (d, J=2.0 Hz, 1H), 7.58-7.48 (m, 3H), 7.41-7.33 (m, 3H), 7.09 (d, J=8.0 Hz, 2H), 6.72 (d, J=8.8 Hz, 1H), 6.46 (s, 1H), 6.26 (s, 1H), 5.75 (s, 1H), 3.76 (d, J=6.8 Hz, 2H), 3.62 (d, J=13.6 Hz, 2H), 3.53 (s, 2H), 3.38-3.21 (m, 5H), 3.04 (d, J=5.6 Hz, 2H), 2.68 (d, J=10.0 Hz, 2H), 2.56 (t, J=5.2 Hz, 3H), 2.36 (s, 2H), 2.00 (s, 2H), 1.43 (t, J=6.0 Hz, 2H), 1.25-1.16 (m, 1H), 0.93 (s, 6H).

### Synthesis of (2S,4R)-1-((S)-15-(tert-butyl)-2,2,3,3-tetramethyl-13-oxo-4-oxa-7,14-diaza-3-silahexadecan-16-oyl)-4-hydroxy-N—((S)-1-(4-(4-methylthiazol-5-yl)phenyl)ethyl)pyrrolidine-2-carboxamide (Intermediate PN)

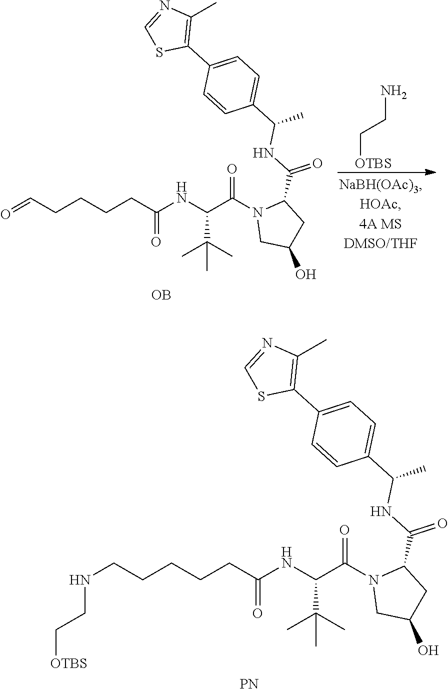

To a solution of 2-[tert-butyl(dimethyl)silyl]oxyethanamine (189 mg, 1.08 mmol, CAS #101711-55-1) in DMSO (2 mL) and THF (1 mL) was added (2S,4R)-1-[(2S)-3,3-dimethyl-2-(6-oxohexanoylamino)butanoyl]-4-hydroxy-N-[(1S)-1-[4-(4-methylthiazol-5-yl)phenyl]ethyl]pyrrolidine-2-carboxamide (300 mg, 539 μmol, Intermediate OB), 4 Å molecular sieves (50 mg) and HOAc (97.0 mg, 1.62 mmol). The mixture was then stirred at 25° C. for 12 hrs. Then NaBH(OAc)₃ (228 mg, 1.08 mmol) was added and the mixture was stirred at 25° C. for 2 hrs. On completion, the reaction mixture was filtered to give a filtrate. The filtrate was purified by reversed-phase HPLC (0.8 g/L ammonium bicarbonate) to give the tittle compound (130 mg, 30% yield) as a white solid. LC-MS (ESI⁺) m/z 716.6 (M+H)⁺.

### Synthesis of 4-((3,3-Diethoxypropyl)amino)-3-((trifluoromethyl)sulfonyl)benzenesulfonamide (Intermediate PO)

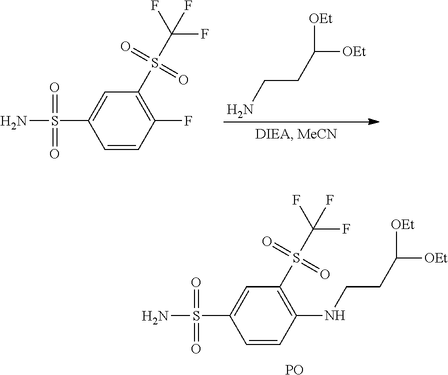

To a solution of 4-fluoro-3-((trifluoromethyl)sulfonyl)benzenesulfonamide (209 mg, 679 μmol, CAS #1027345-08-9) in MeCN (2 mL) was added DIEA (263 mg, 2.04 mmol, 355 uL) and 3,3-diethoxypropan-1-amine (100 mg, 679 μmol, CAS #41365-75-7). The mixture was stirred at 50° C. for 2 hrs. On completion, the mixture was concentrated to give the title compound (300 mg) as a white solid. LC-MS (ESI⁺) m/z 457.1 (M+Na)⁺.

### Synthesis of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-oxopropyl)amino)-3-((trifluoromethyl)sulfonyl)phenyl)sulfonyl)benzamide (Intermediate PP)

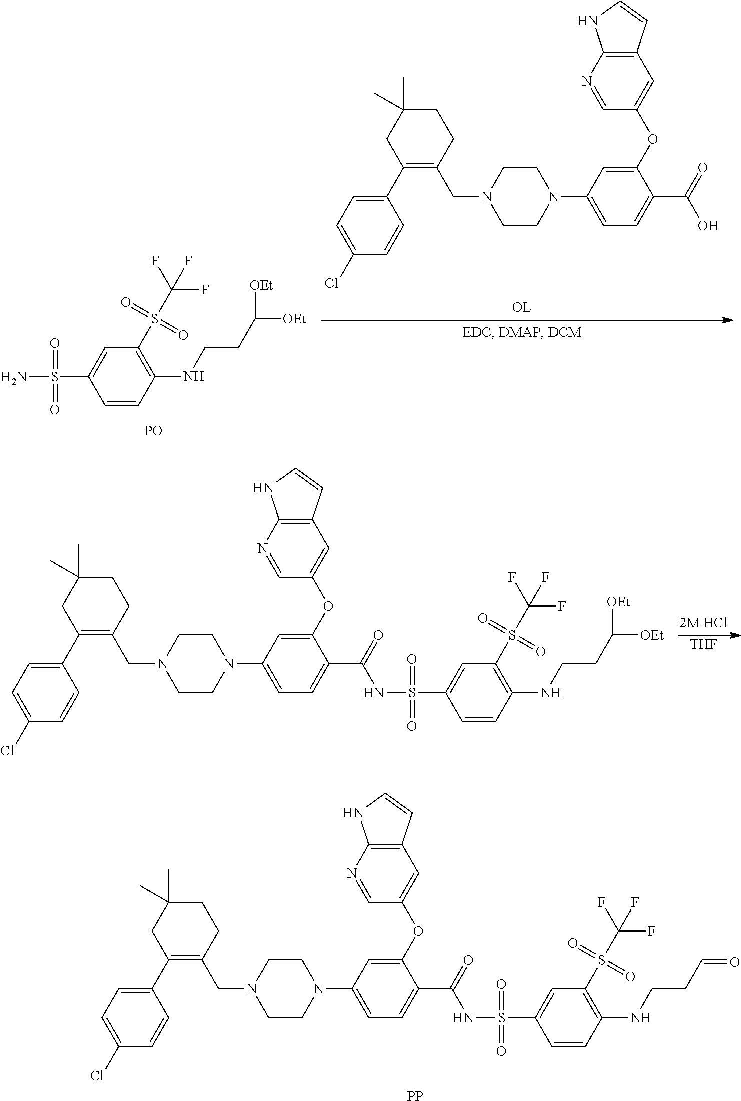

### Step 1—2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3,3-diethoxypropyl)amino)-3-((trifluoromethyl)sulfonyl)phenyl)sulfonyl)benzamide

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (4 g, 7.00 mmol, Intermediate OL) in dichloromethane (40 mL) was added DMAP (2.57 g, 21.0 mmol), then EDC (2.72 g, 17.5 mmol, 3.10 mL) and 4-((3,3-diethoxypropyl)amino)-3-((trifluoromethyl)sulfonyl)benzenesulfonamide (3.04 g, 7.00 mmol, Intermediate PO) was added. The mixture was stirred at 25° C. for 1 hr. On completion, the mixture was concentrated to give the crude product. The product was purified by reversed-phase HPLC (0.1% FA condition) to give the title compound (5.5 g, 80% yield) as a white solid. LC-MS (ESI⁺) m/z 989.0 (M+H)⁺.

### Step 2—2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-oxopropyl)amino)-3-((trifluoromethyl)sulfonyl)phenyl)sulfonyl)benzamide

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3,3-diethoxypropyl)amino)-3-((trifluoromethyl)sulfonyl)phenyl)sulfonyl)benzamide (1.6 g, 1.62 mmol) in THF (10 mL) was added HCl (2 M, 10 mL). The mixture was then stirred at 40° C. for 2 hrs. On completion, the mixture was adjusted pH to 7 with NaHCO₃ solution, then extracted with dichloromethane (30 mL×3). The combined organic layer was dried over anhydrous Na₂SO₄, filtered and the filtrate was concentrated to give the title compound (800 mg, 25% yield) as a brown solid. LC-MS (ESI⁺) m/z 913.4 (M+H)⁺.

### Synthesis of Methyl 4-(4-((2-((4-sulfamoyl-2-((trifluoromethyl)sulfonyl)phenyl)amino)ethyl)amino)phenyl)butanoate (Intermediate PO)

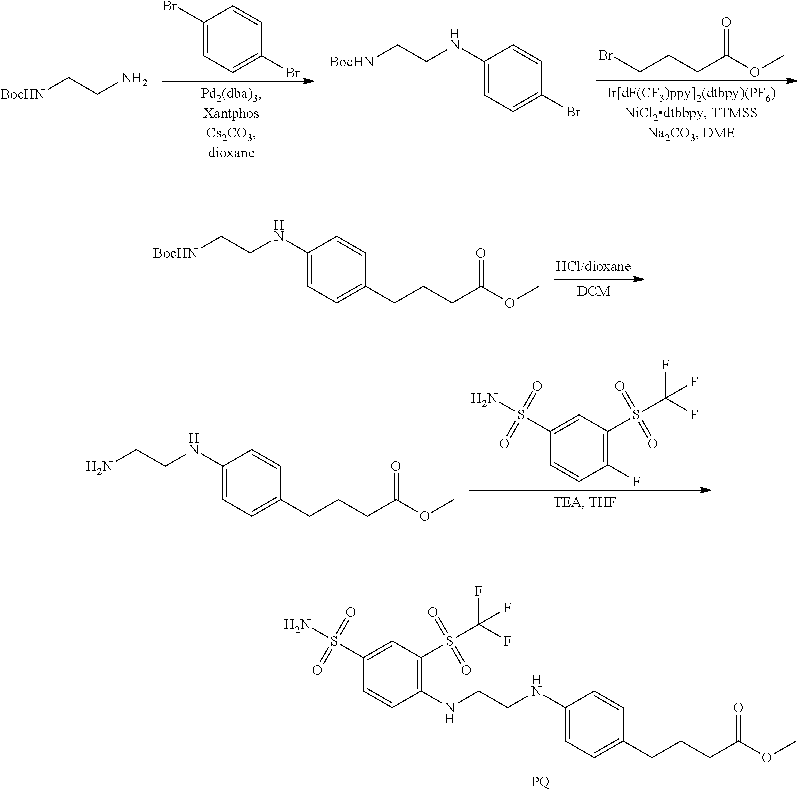

### Step 1—Tert-butyl (2-((4-bromophenyl)amino)ethyl)carbamate

To a solution of tert-butyl N-(2-aminoethyl)carbamate (2.72 g, 16.9 mmol, CAS #57260-73-8) in dioxane (100 mL) was added Cs₂CO₃ (11.0 g, 33.9 mmol), 1,4-dibromobenzene (4 g, 16.9 mmol, 2.17 mL), Xantphos (1.18 g, 2.03 mmol) and Pd₂(dba)₃ (776 mg, 847 μmol). The mixture was then stirred at 90° C. for 12 hrs. On completion, the reaction mixture was concentrated under reduced pressure to give a residue. The residue was purified by column chromatography (SiO₂, Petroleum ether/Ethyl acetate=1/0 to 2/1) to give the title compound (1.8 g, 25% yield) as a white solid. LC-MS (ESI⁺) m/z 260.9 (M+H)⁺.

### Step 2—Methyl 4-(4-((2-((tert-butoxycarbonyl)amino)ethyl)amino)phenyl)butanoate

To a solution of tert-butyl N-[2-(4-bromoanilino)ethyl]carbamate (1.6 g, 5.08 mmol), methyl 4-bromobutanoate (1.19 g, 6.60 mmol), Ir[dF(CF₃)ppy]₂(dtbpy)(PF₆) (56.9 mg, 50.7 μmol), NiCl₂·dtbbpy (30.3 mg, 76.1 μmol), TTMSS (1.26 g, 5.08 mmol, 1.57 mL) and Na₂CO₃ (1.08 g, 10.1 mmol) in DME (25 mL) was degassed and purged with N₂ three times. Then the mixture was stirred at 25° C. for 14 hrs under N₂ atmosphere. On completion, the reaction mixture was concentrated under reduced pressure to give a residue. The residue was purified by prep-HPLC (FA condition) to give the title compound (200 mg, 9% yield, FA) as a black solid. LC-MS (ESI⁺) m/z 281.2 (M+H)⁺.

### Step 3—Methyl 4-(4-((2-aminoethyl)amino)phenyl)butanoate

To a solution of methyl 4-(4-((2-((tert-butoxycarbonyl)amino)ethyl)amino)phenyl)butanoate (250 mg, 743 μmol) in DCM (2 mL) was added HCl/dioxane (2.5 M, 297 uL). The mixture was stirred at 25° C. for 1.5 hrs. On completion, the reaction mixture was concentrated under reduced pressure to give the title compound (175 mg. HCl) as a white solid. LC-MS (ESI+) m/z 237.0 (M+H)⁺.

### Step 4—Methyl 4-(4-((2-((4-sulfamoyl-2-((trifluoromethyl)sulfonyl)phenyl)amino)ethyl)amino)phenyl)butanoate

To a solution of 4-fluoro-3-(trifluoromethylsulfonyl)benzenesulfonamide (221 mg, 719 μmol, CAS #1027345-08-9) and methyl 4-[4-(2-aminoethylamino)phenyl]butanoate (170 mg, 719 μmol) in THF (3 mL) was added TEA (218 mg, 2.16 mmol, 300 uL). The mixture was then stirred at 50° C. and 12 hrs. On completion, the reaction mixture was concentrated under reduced pressure to give a residue. The residue was purified by prep-HPLC (neutral condition) to give the title compound (180 mg, 47% yield) as a white solid. LC-MS (ESI+) m/z 524.0 (M+H)⁺.

### Synthesis of 4-(4-((2-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)ethyl)amino)phenyl)butanoic Acid (Intermediate PR)

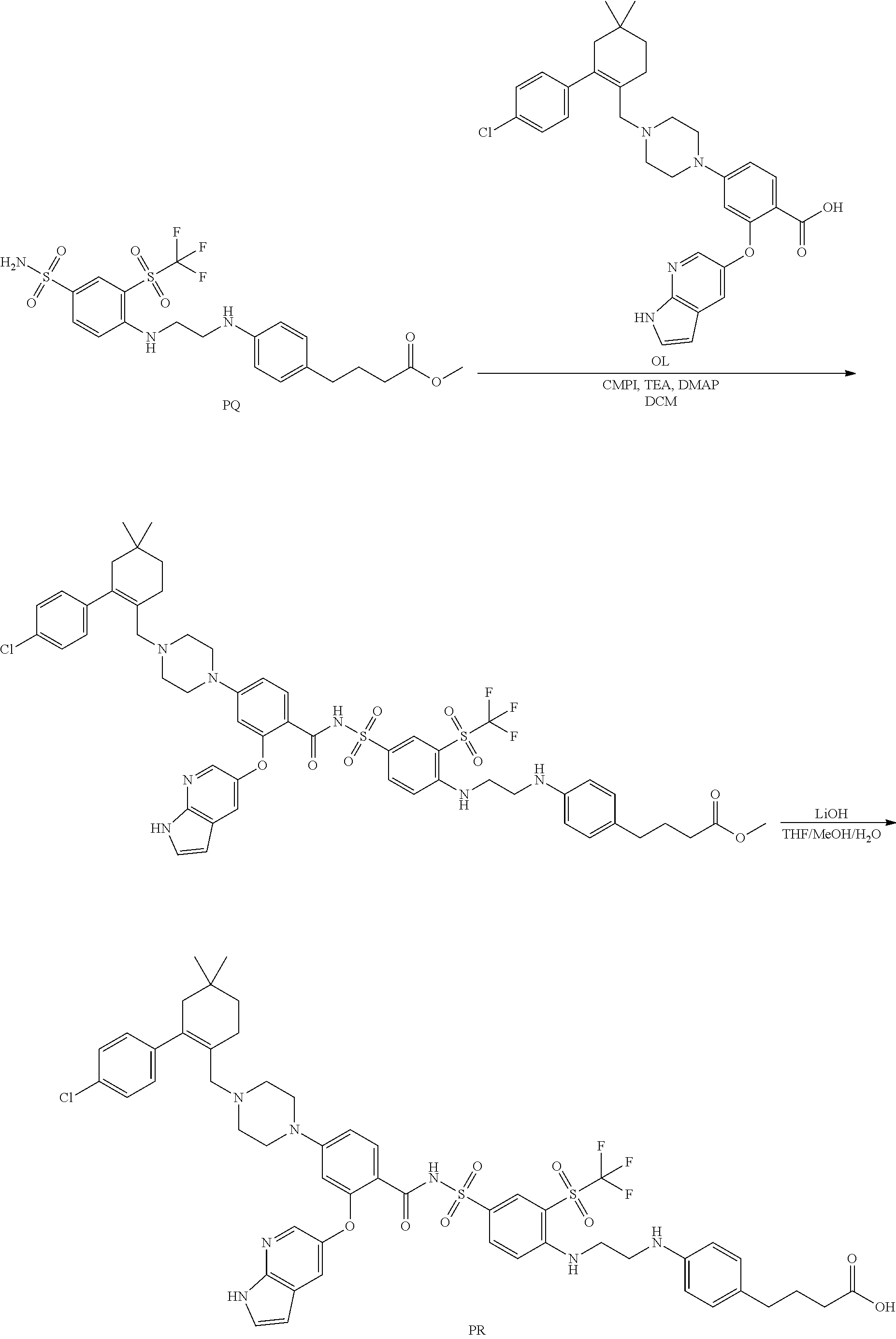

### Step 1—Methyl 4-(4-((2-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)ethyl)amino)phenyl)butanoate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (152 mg, 267 μmol, Intermediate OL) in DCM (3 mL) was added CMPI (102 mg, 401 μmol), TEA (81 mg, 802 μmol, 111 uL), DMAP (32 mg, 267 μmol) and methyl 4-(4-((2-((4-sulfamoyl-2-((trifluoromethyl)sulfonyl)phenyl)amino)ethyl)amino)phenyl)butanoate (140 mg, 267 μmol, Intermediate PQ). The mixture was then stirred at 25° C. for 2 hrs. On completion, the reaction mixture was concentrated under reduced pressure to give a residue. The residue was purified by prep-HPLC (neutral condition) to give the title compound (140 mg, 38% yield) as a white solid. LC-MS (ESI+) m/z 1077.4 (M+H)⁺.

### Step 2—4-(4-((2-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)ethyl)amino)phenyl)butanoic Acid

To a solution of methyl 4-(4-((2-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)ethyl)amino)phenyl)butanoate (140 mg, 130 μmol) in THF (2 mL), MeOH (0.5 mL) and H₂O (0.5 mL) was added LiOH·H₂O (16.3 mg, 390 μmol). The mixture was then stirred at 40° C. for 12 hrs. On completion, the reaction mixture was concentrated under reduced pressure to give a residue. The residue was purified by prep-HPLC (neutral condition) to give the title compound (100 mg, 71% yield) as a white solid. LC-MS (ESI+) m/z 571.3 (M+H)⁺.

### Synthesis of (2S,4R)-1-((S)-2-(2-aminoacetamido)-3,3-dimethylbutanoyl)-4-hydroxy-N—((S)-1-(4-(4-methylthiazol-5-yl)phenyl)ethyl)pyrrolidine-2-carboxamide (Intermediate PS)

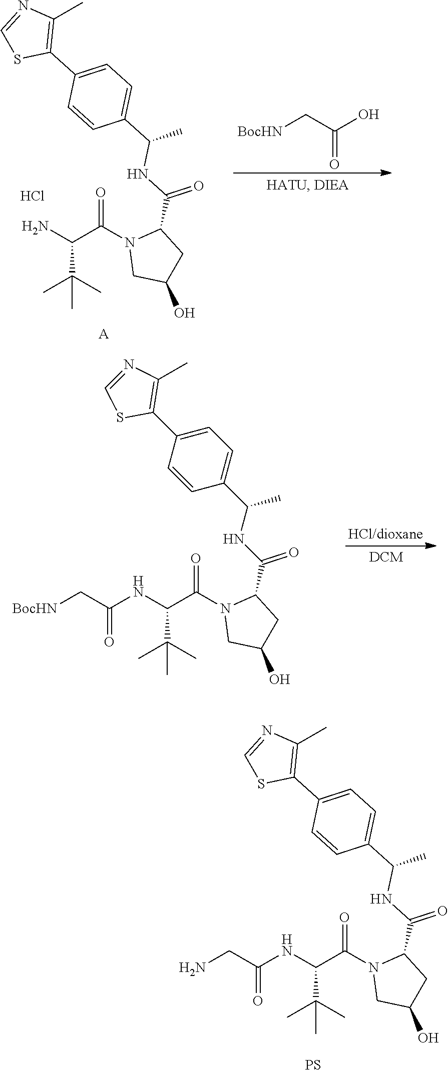

### Step 1—Tert-butyl (2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)carbamate

To a solution of (tert-butoxycarbonyl)glycine (164 mg, 935 μmol, CAS #4530-20-5) in DMF (10 mL) was added HATU (285 mg, 748 μmol) and DIEA (806 mg, 6.24 mmol, 1.09 mL). Then (2S,4R)-1—((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (300 mg, 624 μmol, HCl, Intermediate A) was added and the mixture was stirred at 0° C. for 10 min. On completion, the mixture was diluted with H₂O (50 mL), and extracted with ethyl acetate (30 mL×3). The combined organic layer was washed with brine (20 mL×3), dried over anhydrous Na₂SO₄, filtered and concentrated to give the title compound (450 mg) as a yellow gum. LC-MS (ESI⁺) m/z 602.5 (M+H)⁺.

### Step 2—(2S,4R)-1-((S)-2-(2-aminoacetamido)-3,3-dimethylbutanoyl)-4-hydroxy-N—((S)-1-(4-(4-methylthiazol-5-yl)phenyl)ethyl)pyrrolidine-2-carboxamide

To a solution of tert-butyl (2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)carbamate (400 mg, 665 μmol) in dichloromethane (10 mL) was added HCl/dioxane (4 M, 2.5 mL). The mixture was stirred at 20° C. for 1 hr. On completion, the mixture was concentrated to give the title compound (400 mg, HCl) as a yellow solid. LC-MS (ESI⁺) m/z 502.4 (M+H)⁺.

### Synthesis of 4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoic Acid (Intermediate PT)

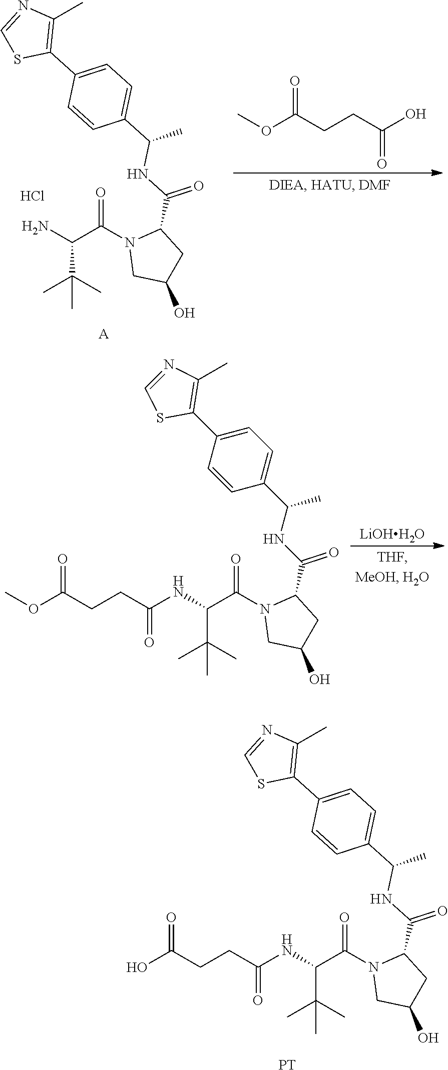

### Step 1—Methyl 4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoate

To a solution of 4-methoxy-4-oxobutanoic acid (302 mg, 2.3 mmol, CAS #3878-55-5) in DMF (15 mL) was added DIEA (2.9 g, 22.9 mmol), HATU (956 mg, 2.5 mmol) and (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride (1.1 g, 2.3 mmol, Intermediate A). The mixture was then stirred at 0° C. for 10 minutes. On completion, the reaction mixture was diluted with water (45 mL) and extracted by ethyl acetate (3×30 mL). The combined organic layers were washed by brine (90 mL) and dried over anhydrous sodium sulfate, filtered and the filtrate was concentrated in vacuo to give the crude residue. Then the crude residue was purified by column chromatography (SiO₂, Petroleum ether:Ethyl acetate=20/1 to 10/1) to give the title compound (1.22 g, 76% yield) as a white solid. LC-MS (ESI+) m/z 559.4 (M+H)⁺.

### Step 2—4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoic Acid

To a solution of methyl 4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoate (1.2 g, 2.2 mmol) in THF (5 mL), MeOH (5 mL) and H₂O (2 mL) was added LiOH·H₂O (901 mg, 21.5 mmol). The mixture was then stirred at 50° C. for 1 hr. On completion, the reaction mixture was concentrated in vacuo to give the crude residue. Then the crude residue was purified by reversed-phase HPLC (0.1% FA condition) to give the title compound (750 mg, 62% yield) as a white solid. LC-MS (ESI⁺) m/z 545.4 (M+H)⁺.

### Synthesis of Ethyl 1-(4-(methyl (3-((4-sulfamoyl-2-((trifluoromethyl)sulfonyl)phenyl)amino)propyl)amino)phenyl)piperidine-4-carboxylate (Intermediate PU)

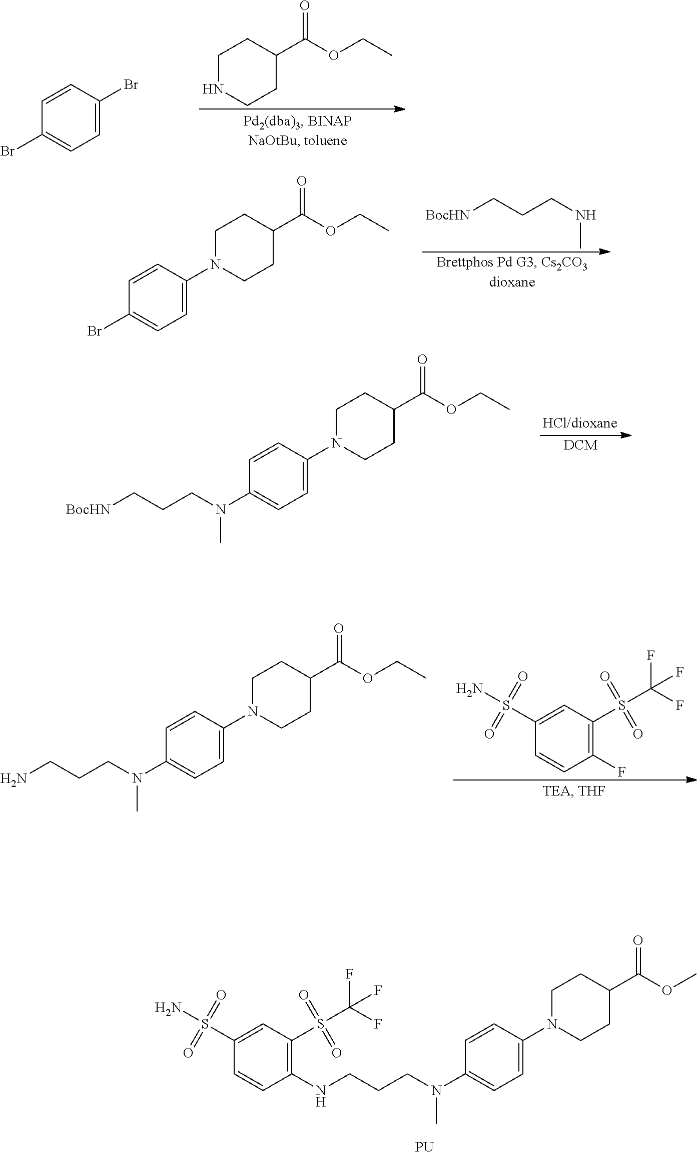

### Step 1—Ethyl 1-(4-bromophenyl)piperidine-4-carboxylate

A mixture of 1,4-dibromobenzene (3 g, 12.7 mmol, CAS #106-37-6), ethyl piperidine-4-carboxylate (2.20 g, 13.9 mmol, CAS #1126-09-6), Pd₂(dba)₃ (419 mg, 457 μmol), BINAP (712 mg, 1.14 mmol) and NaOtBu (1.65 g, 17.1 mmol) in toluene (50 mL) was degassed and purged with N₂ three times. Then the mixture was stirred at 85° C. for 1.5 hrs under N₂ atmosphere. On completion, the reaction mixture was filtered and the filtrate was concentrated in vacuo to give the crude residue. The residue was purified by column chromatography (SiO₂, Petroleum ether/Ethyl acetate=1/0 to 20/1) to give the title compound (1.5 g, 35% yield,) as a yellow oil. LC-MS (ESI⁺) m/z 314.3 (M+H)⁺.

### Step 2—Ethyl 1-(4-((3-((tert-butoxycarbonyl)amino)propyl)(methyl)amino)phenyl)piperidine-4-carboxylate

A mixture of ethyl 1-(4-bromophenyl)piperidine-4-carboxylate (1.1 g, 3.52 mmol), tert-butyl (3-(methylamino)propyl)carbamate (995 mg, 5.29 mmol, CAS #442514-22-9), BrettPhos Pd G3 (319 mg, 352 μmol, CAS #1470372-59-8), Cs₂CO₃ (2.30 g, 7.05 mmol) in dioxane (30 mL) was degassed and purged with N₂ three times. Then the mixture was stirred at 85° C. for 15 hrs under N₂ atmosphere. On completion, the reaction mixture was filtered and the filtrate was concentrated in vacuo to give the crude residue. The residue was purified by column chromatography (SiO₂, Petroleum ether/Ethyl acetate=1/0 to 1/1) to give the title compound (780 mg, 31% yield) as a yellow solid. LC-MS (ESI⁺) m/z 420.5 (M+H)⁺.

### Step 3—Ethyl 1-(4-((3-aminopropyl)(methyl)amino)phenyl)piperidine-4-carboxylate

To a solution of ethyl 1-(4-((3-((tert-butoxycarbonyl)amino)propyl)(methyl)amino)phenyl)piperidine-4-carboxylate (780 mg, 1.86 mmol) in DCM (10 mL) was added HCl/dioxane (4 M, 1 mL). The mixture was stirred at 20° C. for 2 hrs. On completion, the reaction mixture was concentrated in vacuo to give the crude residue. The residue was purified by reversed-phase HPLC (0.1% FA condition) to give a compound (350 mg, 56% yield, FA) as a pink solid: LC-MS (ESI⁺) m/z 319.8 (M+H)⁺.

### Step 4—Ethyl 1-(4-(methyl (3-((4-sulfamoyl-2-((trifluoromethyl)sulfonyl)phenyl)amino)propyl)amino)phenyl)piperidine-4-carboxylate

To a solution of ethyl 1-(4-((3-aminopropyl)(methyl)amino)phenyl)piperidine-4-carboxylate (350 mg, 957 μmol, FA) and 4-fluoro-3-((trifluoromethyl)sulfonyl)benzenesulfonamide (294 mg, 957 μmol, CAS #1027345-08-9) in THF (8 mL) was added TEA (484 mg, 4.79 mmol). The mixture was stirred at 60° C. for 2 hrs. On completion, the reaction mixture was concentrated under reduced pressure to give a residue. The residue was purified by column chromatography (SiO₂, Petroleum ether/Ethyl acetate=1/1 to 0/1) to give the title compound (410 mg, 67% yield) as a yellow solid. LC-MS (ESI⁺) m/z 607.2 (M+H)⁺.

### Synthesis of 1-(4-((3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)(methyl)amino)phenyl)piperidine-4-carboxylic Acid (Intermediate PV)

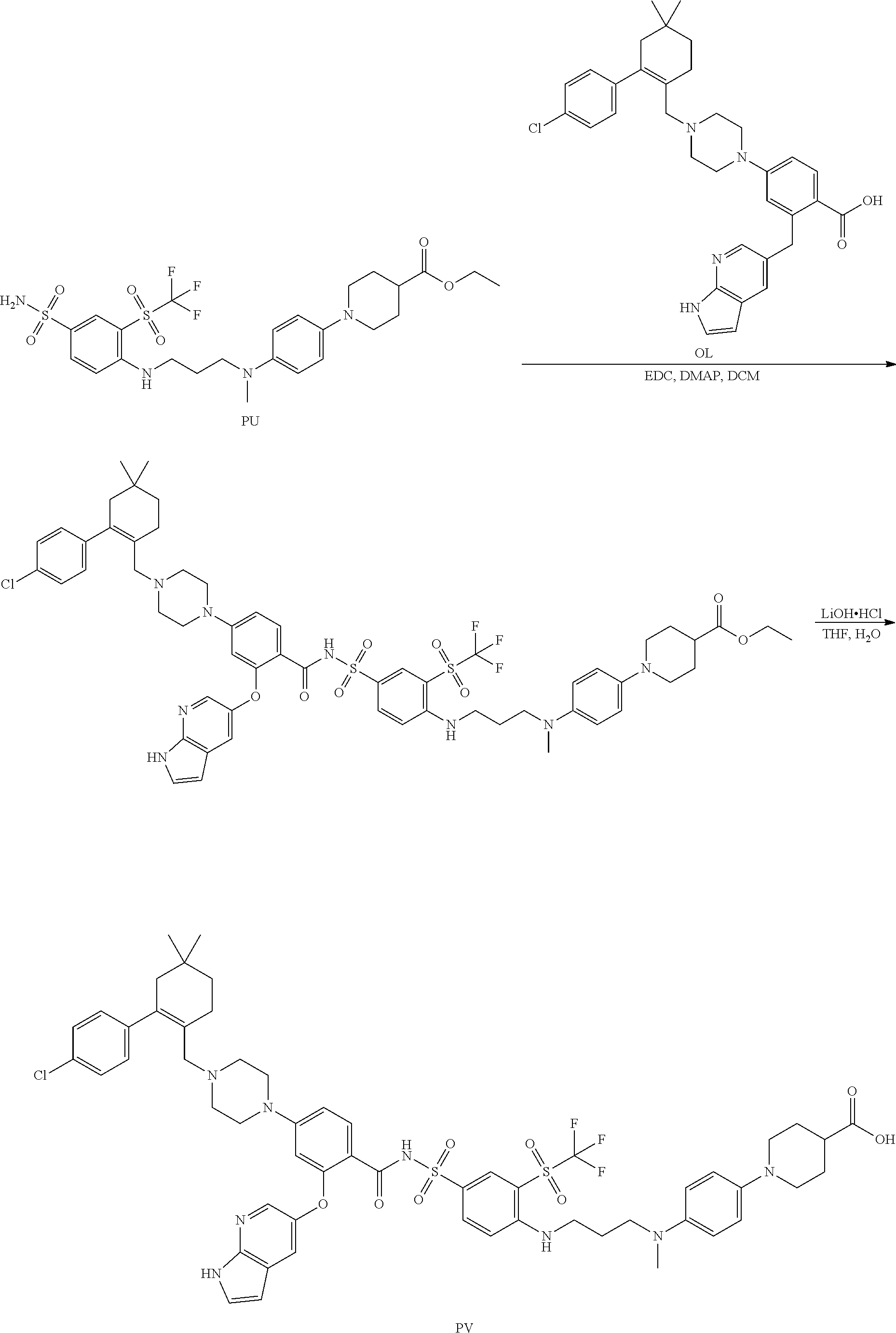

### Step 1—Ethyl 1-(4-((3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)(methyl)amino)phenyl)piperidine-4-carboxylate

To a solution of ethyl 1-(4-(methyl (3-((4-sulfamoyl-2-((trifluoromethyl)sulfonyl)phenyl)amino)propyl)amino)phenyl)piperidine-4-carboxylate (200 mg, 329 μmol, Intermediate PU) and 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (207 mg, 362 μmol, Intermediate OL) in DCM (5 mL) was added EDC (127 mg, 824 μmol) and DMAP (100 mg, 824 μmol). The mixture was stirred at 25° C. for 1 hr. On completion, the reaction mixture was concentrated under reduced pressure to give a residue. The residue was purified by reversed-phase HPLC (0.1% FA condition) to give a title compound (250 mg, 65% yield, FA) as a blue solid. LC-MS (ESI⁺) m/z 580.9 (½ M+H)⁺.

### Step 2—1-(4-((3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)(methyl)amino)phenyl)piperidine-4-carboxylic Acid

To a solution of ethyl 1-(4-((3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)(methyl)amino)phenyl)piperidine-4-carboxylate (250 mg, 215 μmol) in H₂O (1 mL) and THF (4 mL) was added LiOH·H₂O (45.2 mg, 1.08 mmol). The mixture was stirred at 25° C. for 1 hr. On completion, HCl (1N) was added to the reaction mixture until the pH=6.0, then the mixture was diluted with water (25 mL) and extracted with ethyl acetate (3×30 mL). The combined organic layers were dried over anhydrous sodium sulfate, filtered and the filtrate was concentrated in vacuo to give the title compound (240 mg) as a blue solid. LC-MS (ESI⁺) m/z 566.5 (1/2 M+H)⁺.

### Synthesis of Methyl 5-(azetidine-3-carboxamido)pentanoate (Intermediate PW)

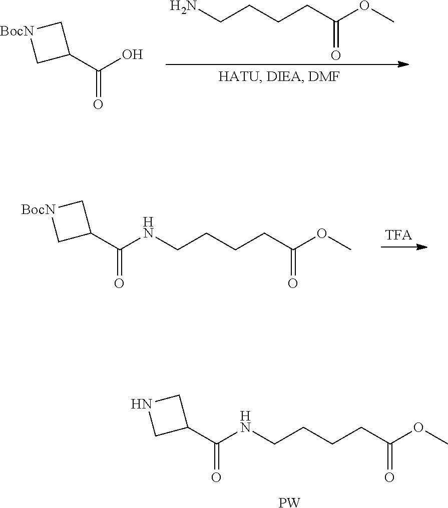

### Step 1—Tert-butyl 3-((5-methoxy-5-oxopentyl)carbamoyl)azetidine-1-carboxylate

To a solution of 1-(tert-butoxycarbonyl)azetidine-3-carboxylic acid (1 g, 4.97 mmol, CAS #142253-55-2) in DMF (10 mL) was added DIEA (6.42 g, 49.7 mmol), HATU (2.08 g, 5.47 mmol) and methyl 5-aminopentanoate (652 mg, 4.97 mmol, CAS #63984-02-1). The mixture was then stirred at 0° C. for 10 mins. On completion, the reaction mixture was diluted by water (20 mL) and extracted by dichloromethane (20×3 mL). The combined organic layers were washed by brine (60 mL), dried over anhydrous sodium sulfate, filtered and the filtrate was concentrated in vacuo to give the crude residue. Then the crude residue was purified by column chromatography (SiO₂, Petroleum ether:Ethyl acetate=1/1 to 0/1) to give the title compound (1.16 g, 67% yield) as a yellow oil. LC-MS (ESI⁺) m/z 259.1 (M+H-55)⁺.

### Step 2—Methyl 5-(azetidine-3-carboxamido)pentanoate

To a solution of tert-butyl 3-((5-methoxy-5-oxopentyl)carbamoyl)azetidine-1-carboxylate (200 mg, 636 μmol) in TFA (2 mL). The mixture was stirred at 25° C. for 1 hr. On completion, the reaction mixture was concentrated in vacuo to give the title compound (130 mg) as a colorless oil. LC-MS (ESI⁺) m/z 215.1 (M+H)⁺.

### Synthesis of 5-(1-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)azetidine-3-carboxamido) pentanoic Acid (Intermediate PX)

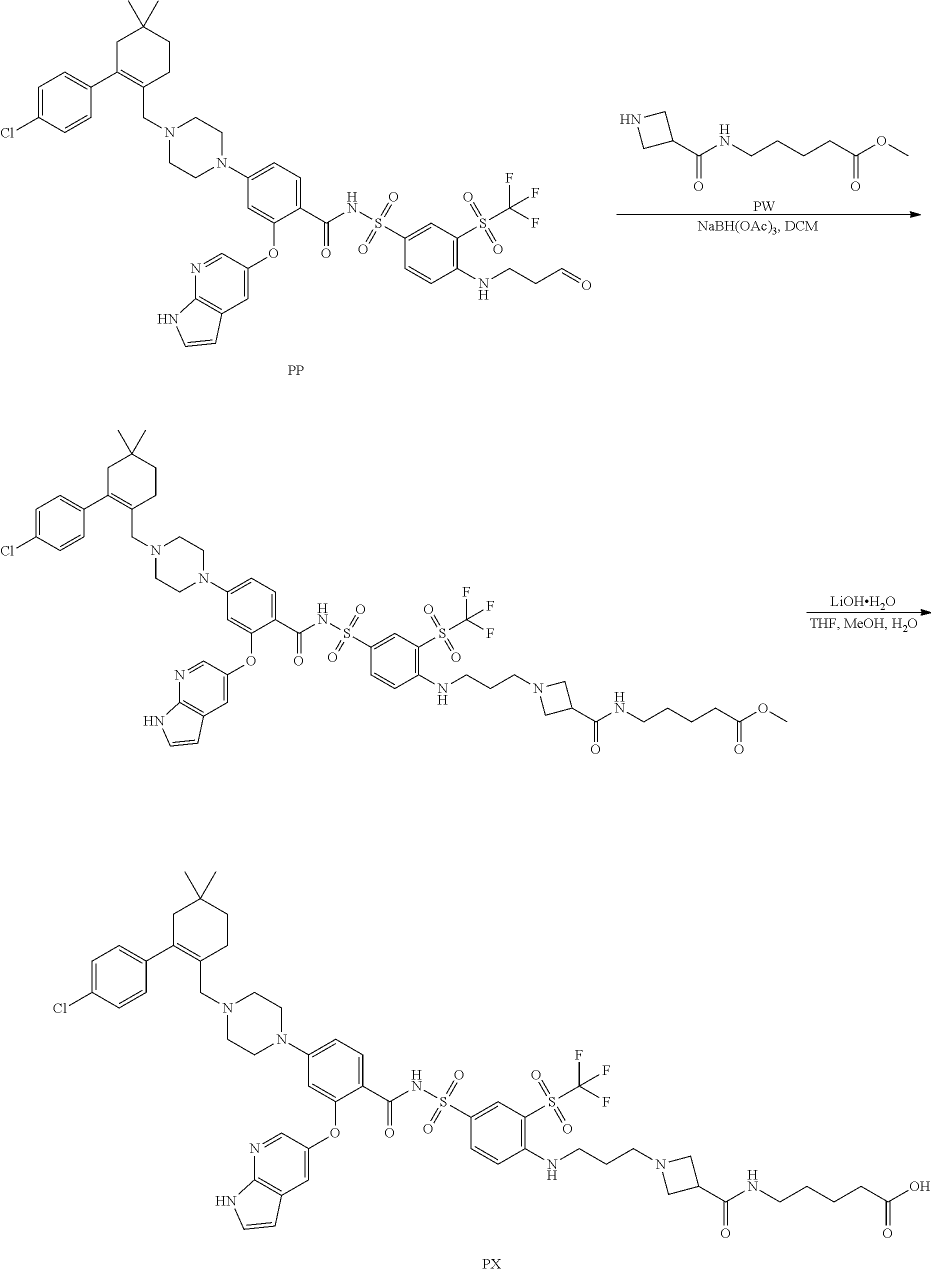

### Step 1—Methyl 5-(1-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)azetidine-3-carboxamido)pentanoate

To a solution of methyl 5-(azetidine-3-carboxamido)pentanoate (46.9 mg, 219 μmol, Intermediate PW) in DCM (2 mL) was added NaBH(OAc)₃ (60.3 mg, 285 μmol) and 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-oxopropyl)amino)-3-((trifluoromethyl)sulfonyl)phenyl)sulfonyl)benzamide (200 mg, 219 μmol, Intermediate PP). The mixture was then stirred at 25° C. for 2 hrs. On completion, the reaction mixture was diluted with water (5 mL) and extracted with ethyl acetate/dichloromethane (3×5 mL). The combined organic layers were washed with brine (15 mL), dried over anhydrous sodium sulfate, filtered and the filtrate was concentrated in vacuo to give the crude residue. Then the crude residue was purified by reversed-phase HPLC (0.1% FA condition) to give the title compound (74 mg, 30% yield) as a white solid. LC-MS (ESI⁺) m/z 1111.3 (M+H)⁺.

### Step 2—5-(1-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)azetidine-3-carboxamido) pentanoic Acid

To a solution of methyl 5-(1-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)azetidine-3-carboxamido)pentanoate (70 mg, 62.9 μmol) in THF (1 mL), MeOH (1 mL) and H₂O (0.4 mL) was added LiOH·H₂O (5.28 mg, 126 μmol). The mixture was stirred at 40° C. for 1 hr. On completion, the reaction mixture was diluted with water (5 mL) and extracted by ethyl acetate/dichloromethane (3×5 mL). The combined organic layers were washed with brine (15 mL), dried over anhydrous sodium sulfate, filtered and the filtrate was concentrated in vacuo to give the title compound (60 mg) as a white solid. LC-MS (ESI⁺) m/z 1097.6 (M+H)⁺.

### Synthesis of 7-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-7-oxoheptanoic Acid (Intermediate PY)

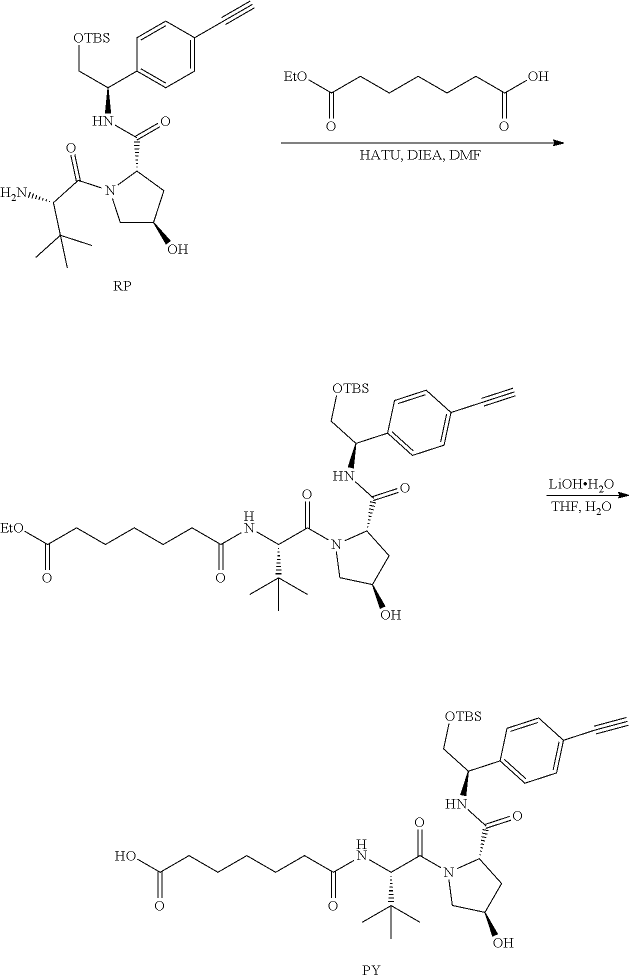

### Step 1—Ethyl 7-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-7-oxoheptanoate

To a solution of (2S,4R)-1-((S)-2-amino-3,3-dimethylbutanoyl)-N—((R)-2-((tert-butyldimethylsilyl)oxy)-1-(4-ethynylphenyl)ethyl)-4-hydroxypyrrolidine-2-carboxamide (500 mg, 997 μmol, Intermediate RP) and 7-ethoxy-7-oxoheptanoic acid (188 mg, 997 μmol, CAS #33018-91-6) in DMF (5 mL) was added HATU (568 mg, 1.49 mmol) and DIEA (644 mg, 4.98 mmol, 868 uL). The mixture was then stirred at 25° C. for 4 h. On completion, the reaction mixture was poured into ice water (5 mL) and extracted with ethyl acetate (5 mL×3). The combined organic layers were washed with brine (5 mL×3), dried over sodium sulfate, filtered and the filtrate was concentrated under reduced pressure to give a residue. The residue was purified by flash silica gel chromatography (ISCO®; 12 g SepaFlash® Silica Flash Column, Eluent of 0˜50% Ethyl acetate/Petroleum ether gradient (@ 70 mL/min) to afford the title compound (380 mg, 56% yield) as a red solid. LC-MS (ESI⁻) m/z 672.6 (M+H)⁺.

### Step 2—7-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-7-oxoheptanoic Acid

To a solution of ethyl 7-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-7-oxoheptanoate (380 mg, 566 μmol) in THF (3 mL) and H₂O (1 mL) was added LiOH·H₂O (71.2 mg, 1.70 mmol). The mixture was then stirred at 25° C. for 12 h. On completion, the reaction mixture was poured into water (4 mL) and extracted with DCM (4 mL×3). The aqueous phase was acidified with 1N HCl to pH=4 then the mixture was extracted with DCM (4 mL×3). The combined organic layers were washed with brine (5 mL×3), dried over Na₂SO₄, filtered and concentrated under reduced pressure to give the title compound (80 mg) as a yellow oil. LC-MS (ESI⁺) m/z 644.6 (M+H)⁺.

### Synthesis of 7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)-7-oxoheptanoic Acid (Intermediate QA)

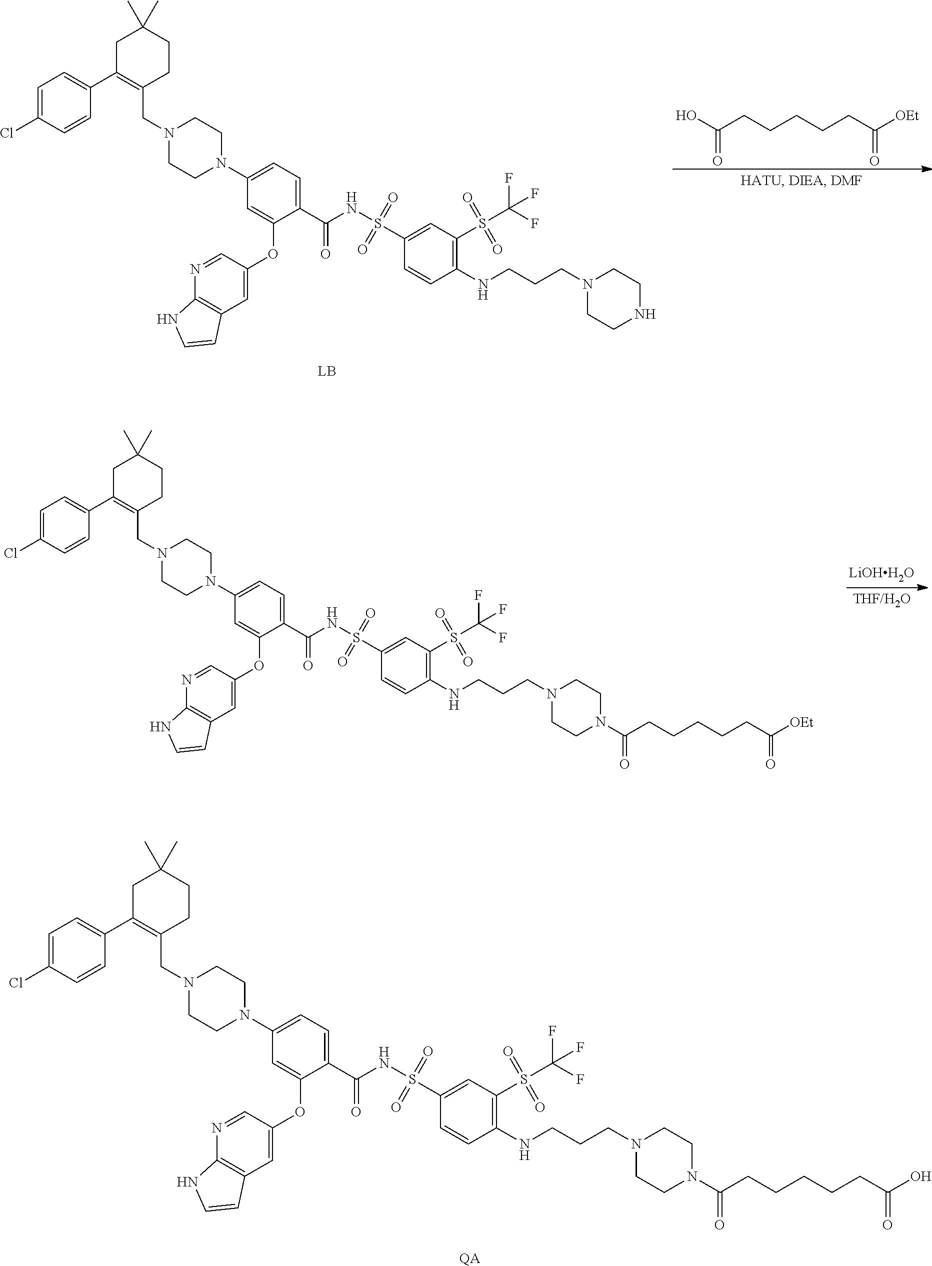

### Step 1—Ethyl 7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)-7-oxoheptanoate

To a solution of 7-ethoxy-7-oxo-heptanoic acid (55.4 mg, 294 μmol, CAS #33018-91-6) in DMF (4 mL) was added HATU (121 mg, 319 μmol) and DIEA (158 mg, 1.23 mmol). The mixture was cooled to 0° C., and 4-[4-[[2-(4-chlorophenyl)-4,4-dimethyl-cyclohexen-1-yl]methyl]piperazin-1-yl]-N-[4-(3-piperazin-1-ylpropylamino)-3-(trifluoromethylsulfonyl)phenyl]sulfonyl-2-(1H-pyrrolo[2,3-b]pyridin-5-yloxy)benzamide (250 mg, 245 μmol, HCl, Intermediate LB) was added. The resulting mixture was stirred at 0° C. for 0.5 hr. On completion, the reaction mixture was diluted with H₂O (8 mL) and extracted with EA (10 mL×3). The combined organic layers were concentrated under reduced pressure to give a residue. The residue was purified by column chromatography (SiO₂, DCM/MeOH=1/0 to 10/1) to give the title compound (400 mg, 85% yield) as a yellow oil. LC-MS (ESI⁺) m/z 1153.5 (M+H)⁺.

### Step 2—7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)-7-oxoheptanoic Acid

To a solution of ethyl 7-[4-[3-[4-[[4-[4-[[2-(4-chlorophenyl)-4,4-dimethyl-cyclohexen-1-yl]methyl]piperazin-1-yl]-2-(1H-pyrrolo[2,3-b]pyridin-5-yloxy)benzoyl]sulfamoyl]-2-(trifluoromethylsulfonyl)anilino]propyl]piperazin-1-yl]-7-oxo-heptanoate (400 mg, 208 μmol) in THF (4 mL) and H₂O (4 mL) was added LiOH·H₂O (43.7 mg, 1.04 mmol). The mixture was stirred at 25° C. for 2 hrs. On completion, the reaction mixture was washed with EA (5 mL×2), and the aqueous phase was added 1M HCl to adjust the pH to 6, and then the mixture was extracted with EA (5 mL×3). The combined organic layers concentrated under reduced pressure to give the title compound (230 mg, 95% yield) as a yellow solid. LC-MS (ESI⁺) m/z 1127.4 (M+H)⁺.

### Synthesis of (2S,4R)-1-((S)-2-amino-3,3-dimethylbutanoyl)-N—((S)-1-(4-ethynyl-3-fluorophenyl)ethyl)-4-hydroxypyrrolidine-2-carboxamide (Intermediate QB)

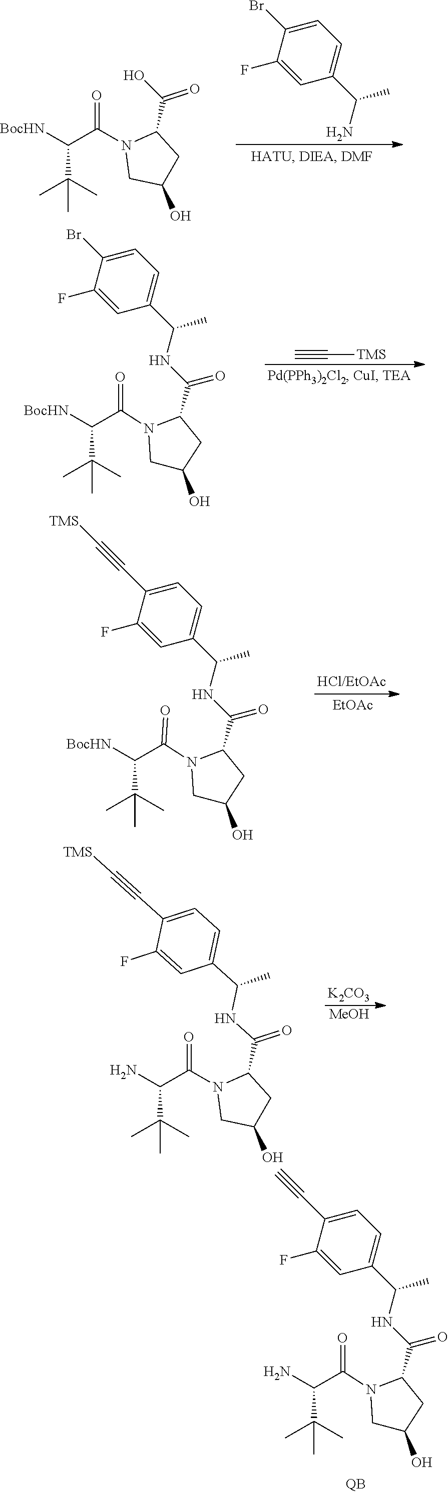

### Step 1—Tert-butyl ((S)-1-((2S,4R)-2-(((S)-1-(4-bromo-3-fluorophenyl)ethyl)carbamoyl)-4-hydroxypyrrolidin-1-yl)-3,3-dimethyl-1-oxobutan-2-yl)carbamate

To a solution of (2S,4R)-1-((S)-2-((tert-butoxycarbonyl)amino)-3,3-dimethylbutanoyl)-4-hydroxypyrrolidine-2-carboxylic acid (400 mg, 1.16 mmol, CAS #1067658-27-8) and HATU (574 mg, 1.51 mmol) in DMF (4 mL) was added(S)-1-(4-bromo-3-fluorophenyl)ethanamine (380 mg, 1.74 mmol, CAS #1241678-53-4) and DIEA (450.32 mg, 3.48 mmol, 607 uL) at 0° C. under N₂. The mixture was then stirred at 25° C. for 12 h. On completion, the reaction mixture was quenched with water (4 mL) at 0° C., extracted with EtOAc (10 mL×3). The combined organic layers were washed with brine (30 mL×2), dried over Na₂SO₄, filtered and concentrated under reduced pressure to give a residue. The residue was purified by column chromatography (SiO₂, Petroleum ether/Ethyl acetate=1/0 to 1/1) to give the title compound (700 mg, 93% yield) as a white solid. ¹H NMR (400 MHz, DMSO-d₆) δ=8.38 (br d, J=7.2 Hz, 1H), 7.62 (t, J=8.0 Hz, 1H), 7.27 (dd, J=1.2, 10.4 Hz, 1H), 7.08 (br d, J=8.4 Hz, 1H), 6.39 (br d, J=9.6 Hz, 1H), 5.10 (br d, J=2.8 Hz, 1H), 4.84 (br t, J=7.2 Hz, 1H), 4.41 (br t, J=8.0 Hz, 1H), 4.28 (br s, 1H), 4.13 (br d, J=8.8 Hz, 1H), 3.62-3.53 (m, 2H), 2.69 (d, J=2.0 Hz, 2H), 2.18 (t, J=8.0 Hz, 1H), 1.94-1.87 (m, 1H), 1.78-1.69 (m, 1H), 1.38 (s, 9H), 0.92 (s, 9H).

### Step 2—Tert-butyl ((S)-1-((2S,4R)-2-(((S)-1-(3-fluoro-4-((trimethylsilyl)ethynyl)phenyl)ethyl)carbamoyl)-4-hydroxypyrrolidin-1-yl)-3,3-dimethyl-1-oxobutan-2-yl)carbamate

To a solution of tert-butyl ((S)-1-((2S,4R)-2-(((S)-1-(4-bromo-3-fluorophenyl)ethyl)carbamoyl)-4-hydroxypyrrolidin-1-yl)-3,3-dimethyl-1-oxobutan-2-yl)carbamate (450 mg, 694 μmol) and ethynyl (trimethyl) silane (682 mg, 6.94 mmol, 962 uL, CAS #1066-54-2) in TEA (10 mL) was added Pd(PPh₃)₂Cl₂ (48.7 mg, 69.4 μmol) and CuI (26.4 mg, 139 μmol). The mixture was then stirred at 85° C. for 12 h under N₂ atmosphere. On completion, the reaction mixture was quenched with water (10 mL) at 20° C., and extracted with EtOAc (20 mL×4). The combined organic layers were washed with brine (100 mL×2), dried over Na₂SO₄, filtered and concentrated under reduced pressure to give a residue. The crude product was purified by reversed-phase HPLC (0.1% FA condition) to give the title compound (230 mg, 59% yield) as a brown solid. LC-MS (ESI⁺) m/z 562.5 (M+H)⁺.

### Step 3—(2S,4R)-1-((S)-2-amino-3,3-dimethylbutanoyl)-N—((S)-1-(3-fluoro-4-((trimethylsilyl)ethynyl)phenyl)ethyl)-4-hydroxypyrrolidine-2-carboxamide

To a solution of tert-butyl ((S)-1-((2S,4R)-2-(((S)-1-(3-fluoro-4-((trimethylsilyl)ethynyl)phenyl)ethyl)carbamoyl)-4-hydroxypyrrolidin-1-yl)-3,3-dimethyl-1-oxobutan-2-yl)carbamate (230 mg, 409 μmol) in EtOAc (2.3 mL) was added HCl/EtOAc (0.8 M, 2.3 mL). The mixture was then stirred at 25° C. for 12 h. On completion, the reaction mixture was concentrated under reduced pressure to give the title compound (230 mg, HCl) as a white solid. LC-MS (ESI⁺) m/z 462.4 (M+H)⁺.

### Step 4—(2S,4R)-1-((S)-2-amino-3,3-dimethylbutanoyl)-N—((S)-1-(4-ethynyl-3-fluorophenyl)ethyl)-4-hydroxypyrrolidine-2-carboxamide

To a solution of (2S,4R)-1-((S)-2-amino-3,3-dimethylbutanoyl)-N—((S)-1-(3-fluoro-4-((trimethylsilyl)ethynyl)phenyl)ethyl)-4-hydroxypyrrolidine-2-carboxamide (230 mg, 462 μmol, HCl) in MeOH (2.3 mL) was added K₂CO₃ (128 mg, 924 μmol). The mixture was then stirred at 25° C. for 1 h. On completion, the reaction mixture was filtered and concentrated under reduced pressure to give the title compound (220 mg) as a yellow solid. LC-MS (ESI+) m/z 390.1 (M+H)⁺.

### Synthesis of 4-(4-((4′-Chloro-5,5-dimethyl-3,4,5,6-tetrahydro-[1,1′-biphenyl]-2-yl)methyl)piperazin-1-yl)-2-(4-methoxyphenoxy)benzoic Acid (Intermediate QC)

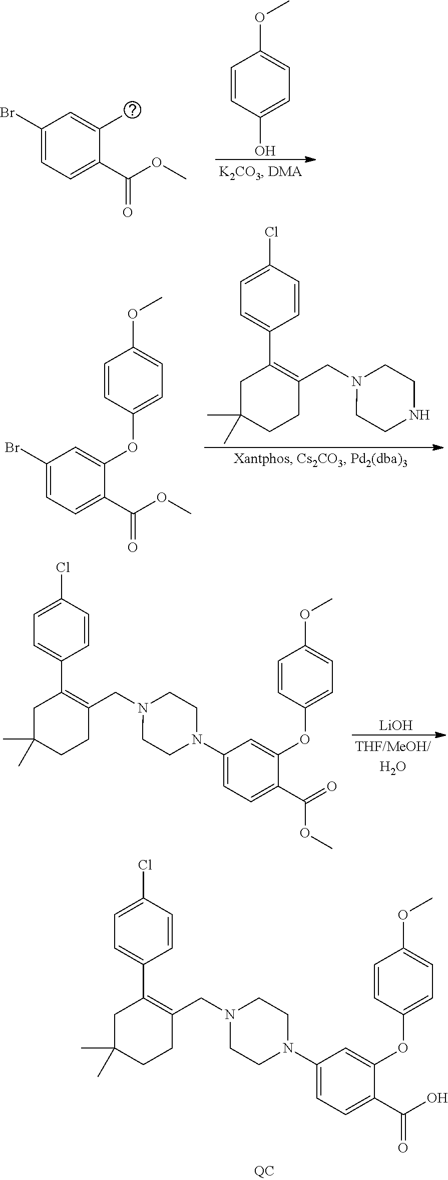

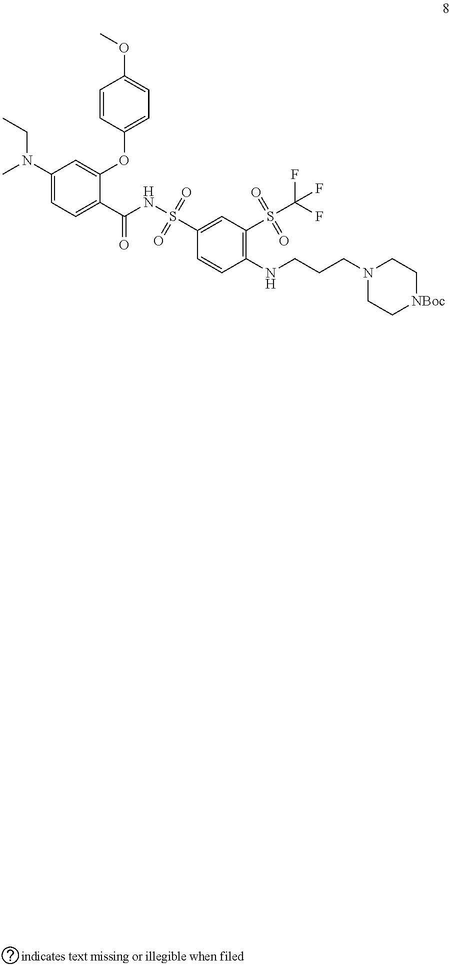 [SMILES: CCN(C)c1ccc(C(=O)NS(=O)(=O)c2ccc(NCCCN3CCN(C(=O)OC(C)(C)C)CC3)c(S(=O)(=O)C(F)(F)F)c2)c(Oc2ccc(OC)cc2)c1]

### Step 1—Methyl 4-bromo-2-(4-methoxyphenoxy)benzoate

To a solution of 4-methoxyphenol (2.66 g, 21.5 mmol, CAS #150-76-5) and methyl 4-bromo-2-fluorobenzoate (5 g, 21.5 mmol, CAS #179232-29-2) in DMA (40 mL) was added K₂CO₃ (5.93 g, 42.9 mmol) at 25° C., then the mixture was stirred at 120° C. for 12 hr. On completion, the reaction mixture was quenched with water (50 mL) and extracted with ethyl acetate (60×3 mL). The extracts were washed with brine (800 mL), dried over anhydrous sodium sulfate, filtered and concentrated in vacuo to give the crude residue. The residue was purified by column chromatography (SiO₂, Petroleum ether/Ethyl acetate=20/1) to give the title compound (4.48 g, 60% yield) as a yellow solid. LC-MS (ESI⁺) m/z 338.8 (M+H)⁺.

### Step 2—Methyl 4-(4-((4′-chloro-5,5-dimethyl-3,4,5,6-tetrahydro-[1,1′-biphenyl]-2-yl)methyl)piperazin-1-yl)-2-(4-methoxyphenoxy)benzoate

To a solution of methyl 4-bromo-2-(4-methoxyphenoxy)benzoate (1.75 g, 5.19 mmol) and 1-((4′-chloro-5,5-dimethyl-3,4,5,6-tetrahydro-[1,1′-biphenyl]-2-yl)methyl)piperazine (1.66 g, 4.66 mmol, HCl, CAS #1228780-72-0) in toluene (10 mL) was added Xantphos (300 mg, 519 μmol), Cs₂CO₃ (5.07 g, 15.6 mmol) and Pd₂(dba)₃ (475 mg, 519 μmol) at 25° C., then the mixture was stirred at 100° C. for 2 hrs. On completion, the reaction mixture was filtered and concentrated in vacuo to give the crude residue. The residue was purified by column chromatography (SiO₂, Petroleum ether/Ethyl acetate=10/1 to 8/1) to give the title compound (2 g, 48% yield,) as a yellow solid. LC-MS (ESI⁺) m/z 575.6 (M+H)⁺.

### Step 3—4-(4-((4′-Chloro-5,5-dimethyl-3,4,5,6-tetrahydro-[1,1′-biphenyl]-2-yl)methyl)piperazin-1-yl)-2-(4-methoxyphenoxy)benzoic Acid

To a solution of methyl 4-(4-((4′-chloro-5,5-dimethyl-3,4,5,6-tetrahydro-[1,1′-biphenyl]-2-yl)methyl)piperazin-1-yl)-2-(4-methoxyphenoxy)benzoate (2.36 g, 4.10 mmol) in THF (20 mL), MeOH (5 mL) and H₂O (5 mL) was added LiOH·H₂O (1.38 g, 32.8 mmol) at 25° C., then the mixture was stirred at 50° C. for 12 hrs. On completion, HCl (1N) was added to the reaction mixture until the pH=3, then the mixture was diluted by water (30 mL) and extracted by ethyl dichloromethane (30×2 mL). The extracts were dried over anhydrous sodium sulfate, filtered and concentrated in vacuo to give the title compound (2 g) as a yellow solid.

### Synthesis of 4-(4-((4′-chloro-5,5-dimethyl-3,4,5,6-tetrahydro-[1,1′-biphenyl]-2-yl)methyl)piperazin-1-yl)-2-(4-methoxyphenoxy)-N-((4-((3-(piperazin-1-yl)propyl)amino)-3-((trifluoromethyl)sulfonyl)phenyl)sulfonyl)benzamide (Intermediate QD)

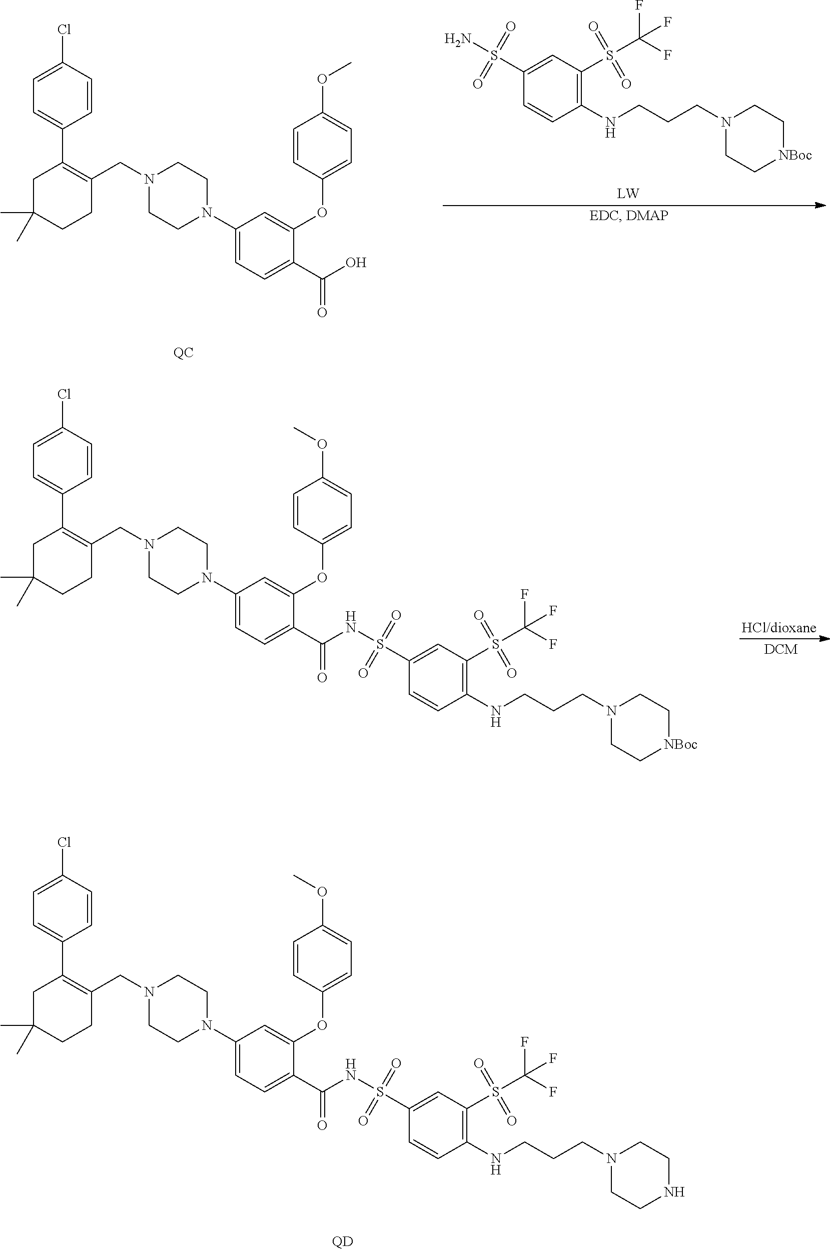

### Step 1—Tert-butyl 4-(3-((4-(N-(4-(4-((4′-chloro-5,5-dimethyl-3,4,5,6-tetrahydro-[1,1′-biphenyl]-2-yl)methyl)piperazin-1-yl)-2-(4-methoxyphenoxy)benzoyl)sulfamoyl)-2-(trifluoromethyl)sulfonyl)phenyl)amino)propyl)piperazine-1-carboxylate

To a solution of 4-(4-((4′-chloro-5,5-dimethyl-3,4,5,6-tetrahydro-[1,1′-biphenyl]-2-yl)methyl)piperazin-1-yl)-2-(4-methoxyphenoxy)benzoic acid (200 mg, 356 μmol, Intermediate QC) in DCM (3 mL) was added CMPI (124 mg, 486 μmol), TEA (98.4 mg, 972 μmol, 135 uL) and DMAP (39.6 mg, 324 μmol) at 25° C. Then tert-butyl 4-(3-((4-sulfamoyl-2-((trifluoromethyl)sulfonyl)phenyl)amino)propyl)piperazine-1-carboxylate mg, 324 μmol, Intermediate LA) was added at 25° C., and the mixture was stirred at 40° C. for 1 hr. On completion, the reaction mixture was concentrated in vacuo to give the crude residue. The residue was purified by column chromatography (SiO₂, Petroleum ether/Ethyl acetate=3/1 to 1/1) to give the title compound (400 mg, 92% yield) as a yellow solid. LC-MS (ESI⁺) m/z 537.5 (M+H)⁺.

### Step 2—4-(4-((4′-Chloro-5,5-dimethyl-3,4,5,6-tetrahydro-[1,1′-biphenyl]-2-yl)methyl)piperazin-1-yl)-2-(4-methoxyphenoxy)-N-((4-((3-(piperazin-1-yl)propyl)amino)-3-((trifluoromethyl)sulfonyl)phenyl)sulfonyl)benzamide

To a solution of tert-butyl 4-(3-((4-(N-(4-(4-((4′-chloro-5,5-dimethyl-3,4,5,6-tetrahydro-[1,1′-biphenyl]-2-yl)methyl)piperazin-1-yl)-2-(4-methoxyphenoxy)benzoyl)sulfamoyl)-2-((trifluoromethyl)sulfonyl)phenyl)amino)propyl)piperazine-1-carboxylate (400 mg, 372 μmol) in DCM (4 mL) was added HCl/dioxane (4 M, 0.8 mL) at 25° C., then the mixture was stirred at 25° C. for 1 hr. On completion, the reaction mixture was filtered and concentrated in vacuo to give the title compound (400 mg, HCl) as a yellow solid. LC-MS (ESI⁺) m/z 973.2 (M+H)⁺.

### Synthesis of Ethyl 1-(4-sulfamoylphenyl)piperidine-4-carboxylate (Intermediate QE)

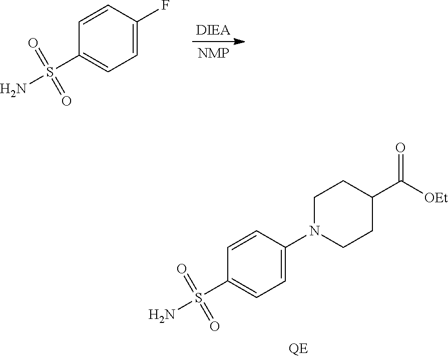 [SMILES: NS(=O)(=O)c1ccc(F)cc1 ; CCOC(=O)C1CCN(c2ccc(S(N)(=O)=O)cc2)CC1]

A mixture of 4-fluorobenzenesulfonamide (10.0 g, 57.1 mmol, CAS #402-46-0), ethyl piperidine-4-carboxylate (89.7 g, 571 mmol, 88.0 mL, CAS #1126-09-6) and DIEA (73.8 g, 571 mmol, 99.4 mL) in NMP (100 mL) was stirred at 140° C. for 12 h. On completion, the reaction mixture was diluted with water (150 mL) and extracted with EtOAc (200 mL×3). The combined organic layers were washed with brine (80 mL×4), dried over Na₂SO₄ and evaporated. The crude product was triturated with PE/EA=1/1 (50 mL) to afford the title compound (8.70 g, 49% yield) as a white solid. ¹H NMR (400 MHz, DMSO-d₆) δ=7.60 (d, J=9.2 Hz, 2H), 7.06-6.98 (m, 4H), 4.07 (q, J=7.2 Hz, 2H), 3.85-3.75 (m, 2H), 2.97-2.87 (m, 2H), 2.63-2.54 (m, 1H), 1.88 (br dd, J=2.8, 13.3 Hz, 2H), 1.65-1.54 (m, 2H), 1.18 (t, J=7.2 Hz, 3H).

### Synthesis of Ethyl 1-(4-aminophenyl)piperidine-4-carboxylate (Intermediate QF)

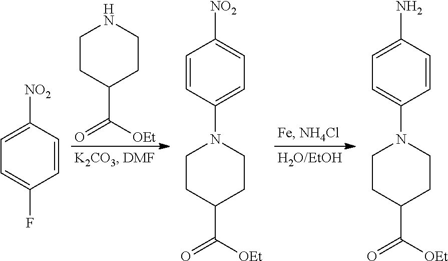

### Step 1—Ethyl 1-(4-nitrophenyl)piperidine-4-carboxylate

To a solution of ethyl piperidine-4-carboxylate (4.01 g, 25.5 mmol, CAS #1126-09-6) in DMF (40 mL) was added 1-fluoro-4-nitrobenzene (3 g, 21.3 mmol, CAS #350-46-9) and K₂CO₃ (7.35 g, 53.2 mmol). The mixture was then stirred at 80° C. for 10 hrs. On completion, the reaction mixture was diluted with water (50 mL) and extracted with ethyl acetate (3×30 mL). The combined organic layers were washed with brine (60 mL) and dried over anhydrous sodium sulfate, filtered and the filtrate was concentrated in vacuo to give the compound (6 g) as a yellow solid. LC-MS (ESI⁺) m/z 279.0 (M+H)⁺.

### Step 2—Ethyl 1-(4-aminophenyl)piperidine-4-carboxylate

To a solution of ethyl 1-(4-nitrophenyl)piperidine-4-carboxylate (3 g, 10.8 mmol) in EtOH (40 mL) and H₂O (15 mL) was added Fe (2.41 g, 43.1 mmol) and NH₄Cl (2.31 g, 43.1 mmol). The mixture was then stirred at 50° C. for 12 hrs. On completion, the reaction mixture was quenched with water (50 mL) and extracted with ethyl acetate (90 mL). The combined organic layers were washed with brine (150 mL) and dried over anhydrous sodium sulfate, filtered and the filtrate was concentrated in vacuo to give the residue. The residue was purified by column chromatography (SiO₂, Petroleum ether/Ethyl acetate=20/1 to 10/1) to give the title compound (2 g, 71% yield) as a brown oil. LC-MS (ESI⁺) m/z 249.1 (M+H)⁺.

### Synthesis of 2-((1H-pyrrolo[2,3-b]pyridin-5-yl)oxy)-4-(4-((4′-chloro-4,4-dimethyl-3,4,5,6-tetrahydro-[1,1′-biphenyl]-2-yl)methyl)piperazin-1-yl)benzoic Acid (Intermediate QG)

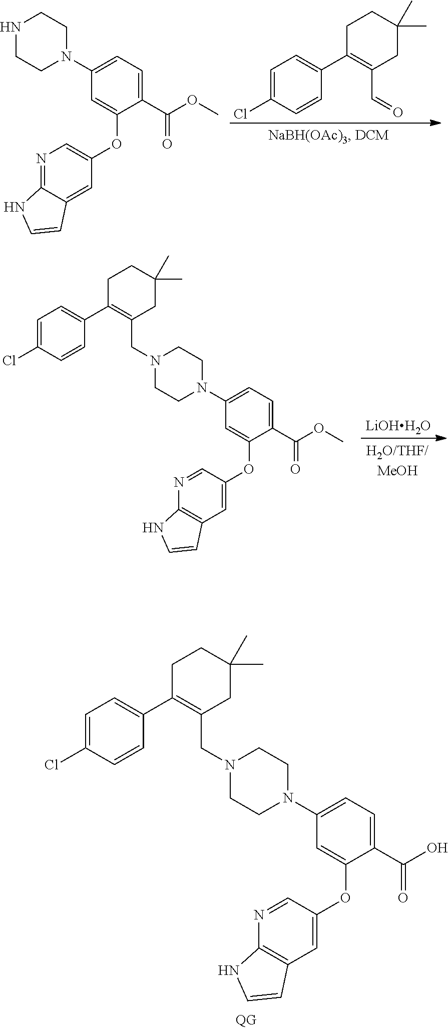

### Step 1—Methyl 2-((1H-pyrrolo[2,3-b]pyridin-5-yl)oxy)-4-(4-((4′-chloro-4,4-dimethyl-3,4,5,6-tetrahydro-[1,1′-biphenyl]-2-yl)methyl)piperazin-1-yl)benzoate

To a solution of methyl 2-((1H-pyrrolo[2,3-b]pyridin-5-yl)oxy)-4-(piperazin-1-yl)benzoate (500 mg, 1.25 mmol, FA, synthesized via Step 1 of Intermediate NQ) and 4′-chloro-4,4-dimethyl-3,4,5,6-tetrahydro-[1,1′-biphenyl]-2-carbaldehyde (312 mg, 1.25 mmol, CAS #1202186-71-7) in DCM (10 mL) was added NaBH(OAc)₃ (265 mg, 1.25 mmol). The mixture was then stirred at 40° C. for 12 hrs. On completion, the reaction mixture was quenched with water (10 mL) and extracted with dichloromethane (2× 10 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=1/0 to 1/1) to give the title compound (390 mg, 52% yield) as a yellow oil. LC-MS (ESI⁺) m/z 585.6 (M+H)⁺.

### Step 2—2-((1H-pyrrolo[2,3-b]pyridin-5-yl)oxy)-4-(4-((4′-chloro-4,4-dimethyl-3,4,5,6-tetrahydro-[1,1′-biphenyl]-2-yl)methyl)piperazin-1-yl)benzoic Acid

To a solution of methyl 2-((1H-pyrrolo[2,3-b]pyridin-5-yl)oxy)-4-(4-((4′-chloro-4,4-dimethyl-3,4,5,6-tetrahydro-[1,1′-biphenyl]-2-yl)methyl)piperazin-1-yl)benzoate (390 mg, 666 μmol) in THF (4 mL) H₂O (2 mL) and MeOH (2 mL) was added LiOH·H₂O (27.9 mg, 666 μmol). The mixture was then stirred at 50° C. for 12 hrs. On completion, the reaction mixture was added HCl (1N) until the pH was 6.0, then the mixture was diluted with water (5 mL) and extracted with dichloromethane (2×10 mL). The combined organic layers were dried over anhydrous sodium sulfate, filtered and the filtrate was concentrated in vacuo to give the title compound (350 mg) as a white solid. LC-MS (ESI⁺) m/z 571.5 (M+H)⁺.

### Synthesis of 2-((1H-pyrrolo[2,3-b]pyridin-5-yl)oxy)-4-(4-((4′-chloro-4,4-dimethyl-3,4,5,6-tetrahydro-[1,1′-biphenyl]-2-yl)methyl)piperazin-1-yl)-N-((4-((3-(piperazin-1-yl)propyl)amino)-3-((trifluoromethyl)sulfonyl)phenyl)sulfonyl)benzamide (Intermediate QH)

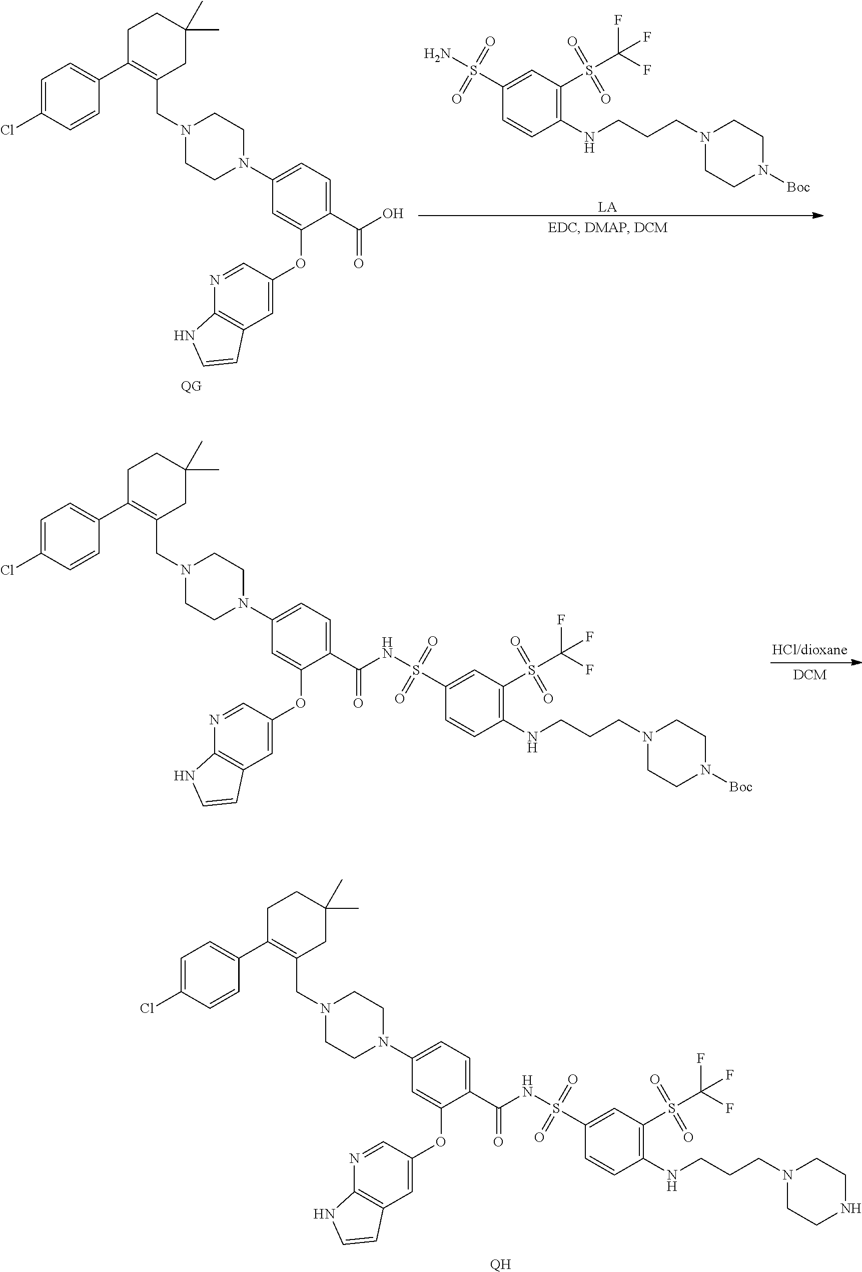

### Step 1—Tert-butyl 4-(3-((4-(N-(2-((1H-pyrrolo[2,3-b]pyridin-5-yl)oxy)-4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazine-1-carboxylate

To a solution of 2-((1H-pyrrolo[2,3-b]pyridin-5-yl)oxy)-4-(4-((4′-chloro-4,4-dimethyl-3,4,5,6-tetrahydro-[1,1′-biphenyl]-2-yl)methyl)piperazin-1-yl)benzoic acid (200 mg, 350 μmol, Intermediate QG) in DCM (4 mL) was added EDC (108 mg, 700 μmol, 123 uL). Then the mixture was stirred for 30 min at rt, and tert-butyl 4-(3-((4-sulfamoyl-2-((trifluoromethyl)sulfonyl)phenyl)amino)propyl)piperazine-1-carboxylate (185 mg, 350 μmol, Intermediate LA) and DMAP (106 mg, 875 μmol) was added. The mixture was then stirred at 25° C. for 2.5 hrs. On completion, the reaction mixture was concentrated under reduced pressure and purified by reversed-phase HPLC (0.8 g/L ammonium bicarbonate) to give the title compound (150 mg, 35% yield) as a white solid. LC-MS (ESI⁺) m/z 1083.8 (M+H)⁺.

### Step 2—2-((1H-pyrrolo[2,3-b]pyridin-5-yl)oxy)-4-(4-((4′-chloro-4,4-dimethyl-3,4,5,6-tetrahydro-[1,1′-biphenyl]-2-yl)methyl)piperazin-1-yl)-N-((4-((3-(piperazin-1-yl)propyl)amino)-3-((trifluoromethyl)sulfonyl)phenyl)sulfonyl)benzamide

To a solution of tert-butyl 4-(3-((4-(N-(2-((1H-pyrrolo[2,3-b]pyridin-5-yl)oxy)-4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazine-1-carboxylate (150 mg, 121 μmol) in DCM (10 mL) was added HCl/dioxane (4 M, 1 mL). The mixture was then stirred at 25° C. for 0.5 hr. On completion, the reaction mixture was concentrated under reduced pressure to give the title compound (120 mg, HCl) as white solid. LC-MS (ESI⁺) m/z 983.4 (M+H)⁺.

### Synthesis of N-((4-((3-(1,4-diazepan-1-yl)propyl)amino)-3-((trifluoromethyl)sulfonyl)phenyl)sulfonyl)-2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzamide (Intermediate QI)

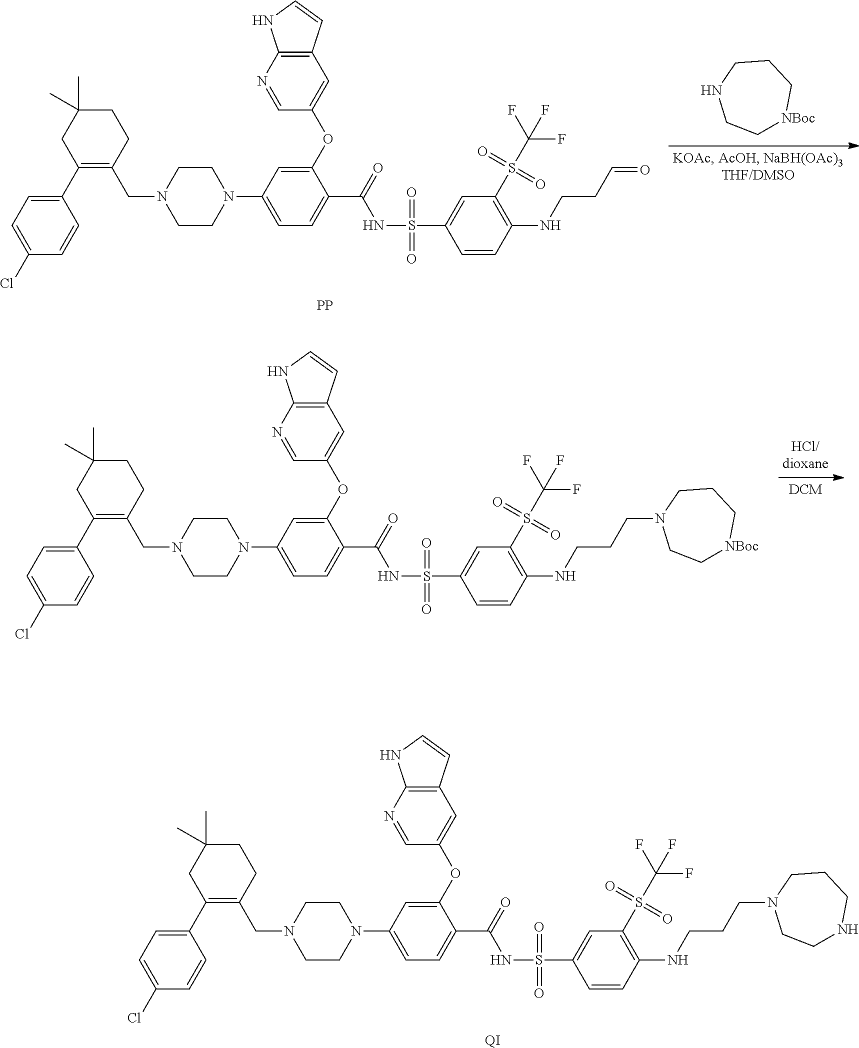

### Step 1—Tert-butyl 4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)-1,4-diazepane-1-carboxylate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-oxopropyl)amino)-3-((trifluoromethyl)sulfonyl)phenyl)sulfonyl)benzamide (1.4 g, 1.53 mmol, Intermediate PP) in THF (10 mL) and DMSO (5 mL) was added tert-butyl 1,4-diazepane-1-carboxylate (460 mg, 2.30 mmol, 451 uL, CAS #112275-50-0), 4 Å molecular sieves (1 g, 1.53 mmol) and HOAc (92.0 mg, 1.53 mmol, 87.7 uL). Then the mixture was stirred at 20° C. for 1 hr. Next, NaBH(OAc) 3 (780 mg, 3.68 mmol) was added and the mixture was stirred at 20° C. for 1 hr. On completion, the mixture was filtered and washed with EtOAc (20 mL×3), then the organic layer was washed with brine, dried over anhydrous Na₂SO₄ and concentrated to give the crude product. The product was purified by column chromatography (SiO₂, Petroleum ether/Ethyl acetate=1/1 to Dichloromethane:MeOH=10:1) to give the title compound (1.1 g, 64% yield) as a white solid. LC-MS (ESI⁺) m/z 1097.3 (M+H)⁺.

### Step 2—N-((4-((3-(1,4-diazepan-1-yl)propyl)amino)-3-((trifluoromethyl)sulfonyl)phenyl)sulfonyl)-2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzamide

To a solution of tert-butyl 4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)-1,4-diazepane-1-carboxylate (1.1 g, 1.00 mmol) in dichloromethane (15 mL) was added HCl/dioxane (4 M, 4 mL). The mixture was then stirred at 25° C. for 0.5 hr. On completion, the mixture was concentrated to give the title compound (1.2 g, HCl) as a white solid. LC-MS (ESI⁺) m/z 997.4 (M+H)⁺.

### Synthesis of (S)-2-((1H-pyrrolo[2,3-b]pyridin-5-yl)oxy)-N-((4-((3-(3-aminopyrrolidin-1-yl)propyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide (Intermediate QJ)

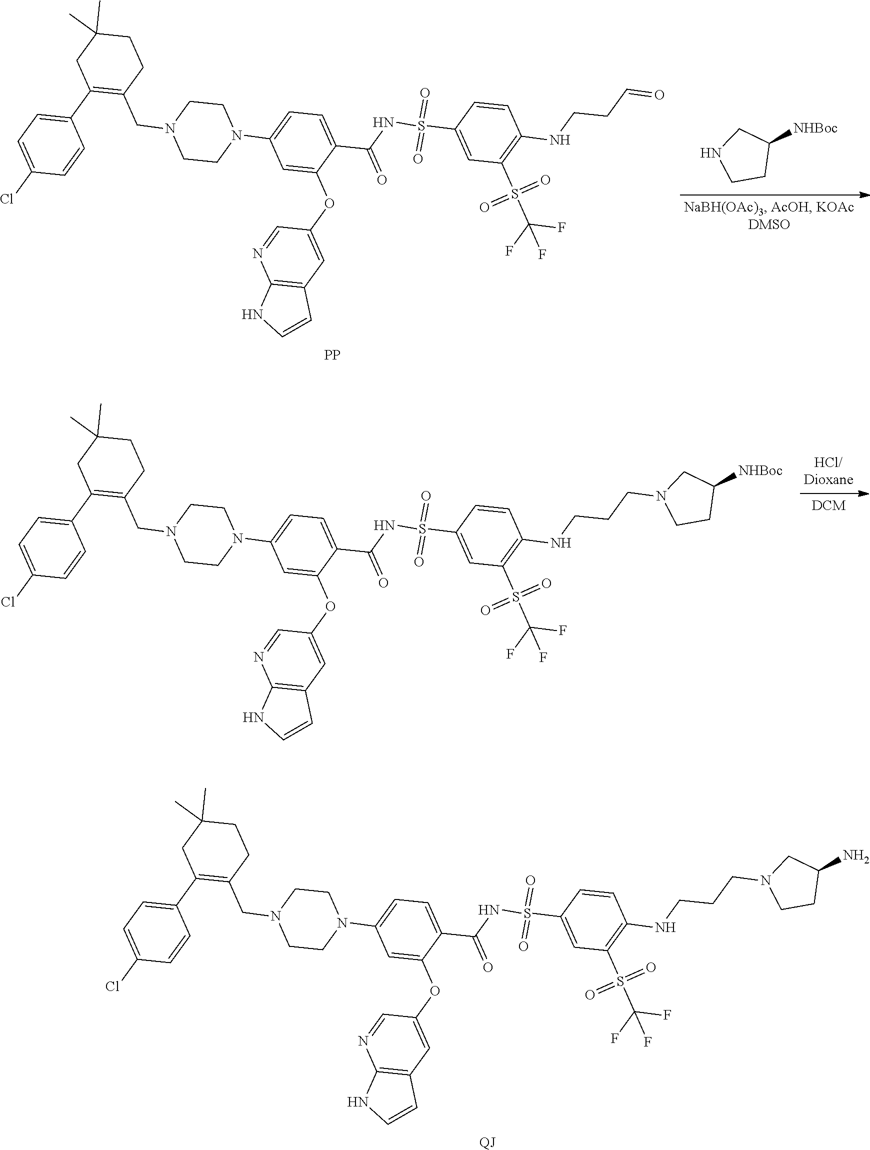

### Step 1—Tert-butyl (S)-(1-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)pyrrolidin-3-yl)carbamate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-oxopropyl)amino)-3-((trifluoromethyl)sulfonyl)phenyl)sulfonyl)benzamide (200 mg, 208 μmol, Intermediate PP) and tert-butyl (S)-pyrrolidin-3-ylcarbamate (58.2 mg, 313 μmol, CAS #122536-76-9) in DMSO (0.5 mL) and THF (1.5 mL) was added dropwise KOAc (61.4 mg, 625 μmol), AcOH (62.6 mg, 1.04 mmol, 59.6 uL), and 4 Å molecular sieves (100 mg) at 25° C. After addition, the mixture was stirred at this temperature for 1 hr, and then NaBH(OAc)₃ (133 mg, 625 μmol) was added at 0° C. The resulting mixture was stirred at 0° C. for 1 hr. On completion, the reaction mixture was filtered and concentrated under reduced pressure to give a residue. The residue was purified by reversed-phase HPLC (0.8 g/L ammonium bicarbonate) to give the title compound (95 mg, 41% yield) as a white solid. LC-MS (ESI⁺) m/z 1083.6 (M+H)⁺.

### Step 2—(S)-2-((1H-pyrrolo[2,3-b]pyridin-5-yl)oxy)-N-((4-((3-(3-aminopyrrolidin-1-yl)propyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide

To a solution of tert-butyl (S)-(1-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)pyrrolidin-3-yl)carbamate (95 mg, 86 μmol) in DCM (10 mL) was added HCl/dioxane (4 M, 1 mL). The mixture was then stirred at 25° C. for 2 hrs. On completion, the reaction mixture was concentrated under reduced pressure to give the title compound (90 mg, HCl) as yellow solid. LC-MS (ESI⁺) m/z 983.6 (M+H)⁺.

### Synthesis of Tert-butyl (R)-6-(3-amino-4-(phenylthio)butyl)-2,6-diazaspiro[3,3]heptane-2-carboxylate (Intermediate OK)

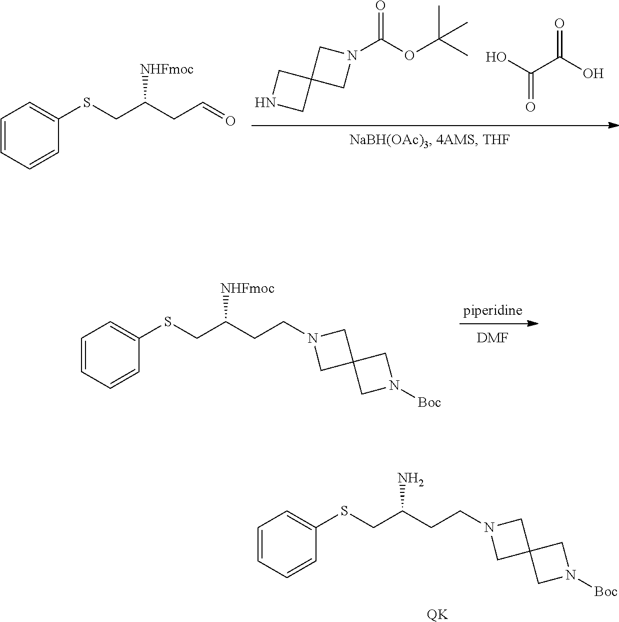

### Step 1—Tert-butyl (R)-6-(3-((((9H-fluoren-9-yl)methoxy) carbonyl)amino)-4-(phenylthio)butyl)-2,6-diazaspiro[3,3]heptane-2-carboxylate

To a solution of (9H-fluoren-9-yl)methyl (R)-(4-oxo-1-(phenylthio)butan-2-yl)carbamate (750 mg, 1.80 mmol, synthesized via Steps 1-3 of Intermediate KK) and tert-butyl 2,6-diazaspiro[3.3]heptane-2-carboxylate oxalate (517 mg, 1.80 mmol, CAS #1227382-01-5) in DCM (10 mL) was added NaBH(OAc)₃ (380 mg, 1.80 mmol) and 4 Å molecular sieves (500 mg). The mixture was then stirred at 25° C. for 12 hrs. On completion, the reaction mixture was the reaction mixture was filtered and the filtrate was concentrated in vacuo to give the crude residue. The residue was purified by column chromatography (SiO₂, Petroleum ether/Ethyl acetate=1/0 to 0/1) to give the title compound (320 mg, 30% yield) as a yellow oil. LC-MS (ESI⁺) m/z 600.1 (M+H)⁺.

### Step 2—Tert-butyl (R)-6-(3-amino-4-(phenylthio)butyl)-2,6-diazaspiro[3,3]heptane-2-carboxylate

To a solution of tert-butyl (R)-6-(3-((((9H-fluoren-9-yl)methoxy) carbonyl)amino)-4-(phenylthio)butyl)-2,6-diazaspiro[3.3]heptane-2-carboxylate (320 mg, 533 μmol) in DMF (2 mL) was added piperidine (45.4 mg, 533 μmol, 52.7 uL). The mixture was then stirred at 25° C. for 12 hrs. On completion, the reaction mixture was filtered and concentrated under reduced pressure to give a residue. The residue was purified by reversed-phase HPLC (0.8 g/L ammonium bicarbonate) to give the title compound (180 mg, 54% yield) as a yellow oil. LC-MS (ESI⁺) m/z 378.0 (M+H)⁺.

### Synthesis of Tert-butyl (R)-6-(4-(phenylthio)-3-((4-sulfamoyl-2-((trifluoromethyl)sulfonyl)phenyl)amino)butyl)-2,6-diazaspiro[3.3]heptane-2-carboxylate (Intermediate QL)

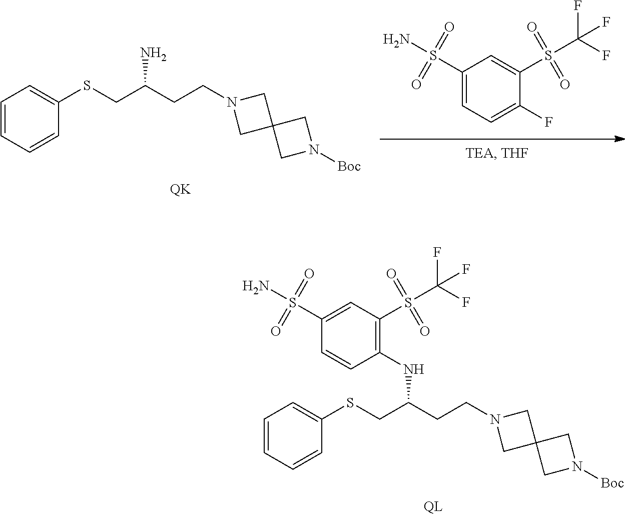

To a solution of tert-butyl (R)-6-(3-amino-4-(phenylthio)butyl)-2,6-diazaspiro[3.3]heptane-2-carboxylate (180 mg, 476 μmol, Intermediate QK) and 4-fluoro-3-((trifluoromethyl)sulfonyl)benzenesulfonamide (146 mg, 476 μmol, CAS #1027345-08-9) in THF (5 mL) was added TEA (144 mg, 1.43 mmol). The mixture was then stirred at 50° C. for 2 hrs. On completion, the reaction mixture was concentrated under reduced pressure to give a residue. The residue was purified by reversed-phase HPLC (0.8 g/L ammonium bicarbonate) to give the title compound (160 mg, 51% yield) as a white solid. LC-MS (ESI⁺) m/z 665.3 (M+H)⁺.

### Synthesis of 4-(4-((R)-(4′-chloro-[1,1′-biphenyl]-2-yl)(hydroxy)methyl)piperidin-1-yl)-N-((4-(((R)-1-(phenylthio)-4-(2,6-diazaspiro[3.3]heptan-2-yl)butan-2-yl)amino)-3-((trifluoromethyl) sulfonyl)phenyl)sulfonyl)benzamide (Intermediate QM)

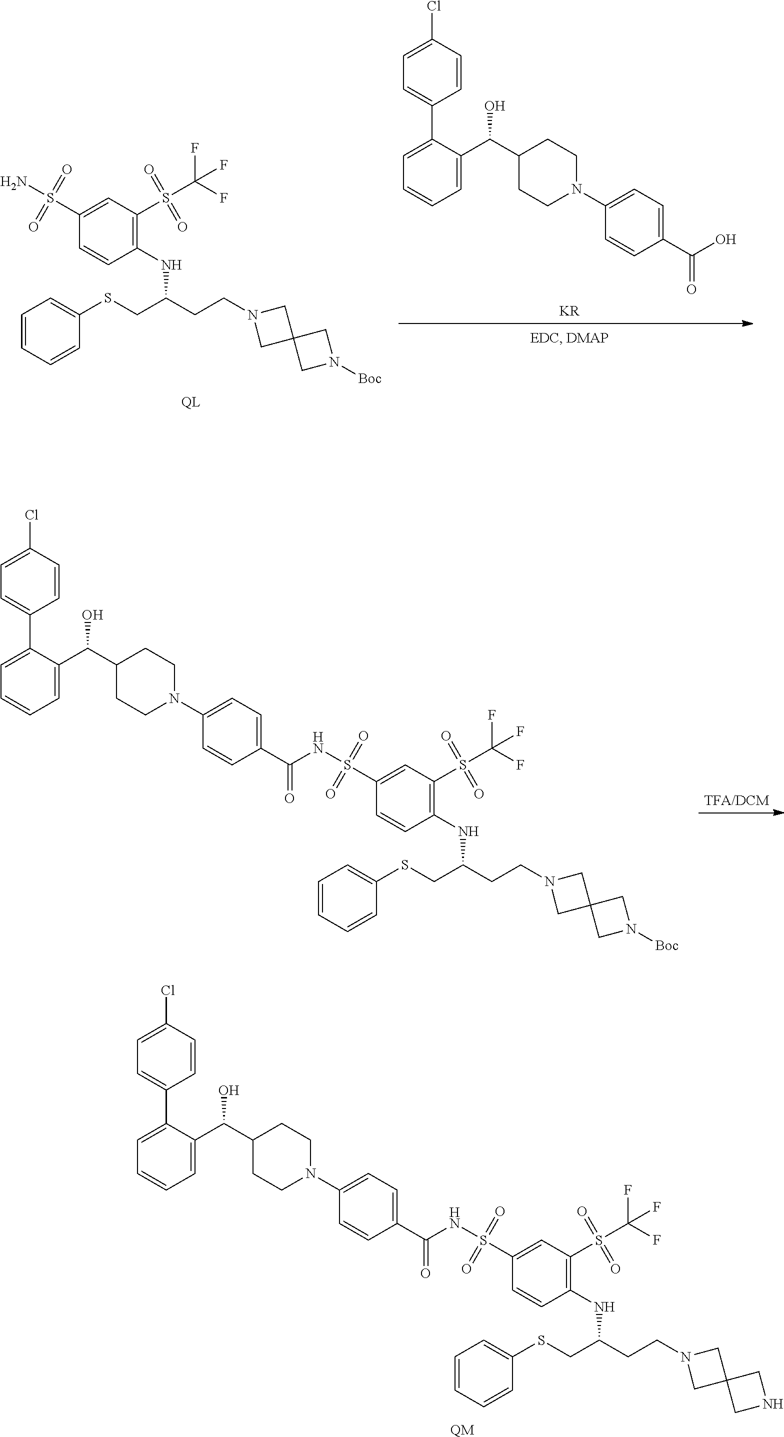

### Step 1—Tert-butyl 6-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenyl)thio)butyl)-2,6-diazaspiro[3.3]heptane-2-carboxylate

To a solution of (R)-4-(4-((4′-chloro-[1,1′-biphenyl]-2-yl)(hydroxy)methyl)piperidin-1-yl)benzoic acid (102 mg, 241 μmol, Intermediate KR) in DCM (5 mL) was added EDC (74.7 mg, 481 μmol, 85.2 uL). Then the mixture was stirred for 30 min, and tert-butyl (R)-6-(4-(phenylthio)-3-((4-sulfamoyl-2-((trifluoromethyl)sulfonyl)phenyl)amino)butyl)-2,6-diazaspiro[3.3]heptane-2-carboxylate (160 mg, 241 μmol, Intermediate QL) and DMAP (73.5 mg, 602 μmol) was added. The mixture was stirred at 40° C. for 2 hrs. On completion, the reaction mixture was concentrated under reduced pressure to give the crude residue. The crude residue was purified by reversed-phase HPLC (0.1% FA condition) to give the title compound (218 mg, 85% yield) as a white solid. LC-MS (ESI⁺) m/z 1068.3 (M+H)⁺.

### Step 2—4-(4-((R)-(4′-chloro-[1,1′-biphenyl]-2-yl)(hydroxy)methyl)piperidin-1-yl)-N-((4-(((R)-1-(phenylthio)-4-(2,6-diazaspiro[3.3]heptan-2-yl)butan-2-yl)amino)-3-((trifluoromethyl)sulfonyl)phenyl)sulfonyl)benzamide

To a solution of tert-butyl 6-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)-2,6-diazaspiro[3.3]heptane-2-carboxylate (210 mg, 197 μmol) in DCM (10 mL) was added TFA (1 mL). The mixture was stirred at 25° C. for 2 hrs. On completion, the reaction mixture was concentrated under reduced pressure to give the title compound (200 mg, TFA) as white solid. LC-MS (ESI⁺) m/z 968.6 (M+H)⁺.

### Synthesis of Tert-butyl (R)-9-(3-amino-4-(phenylthio)butyl)-3,9-diazaspiro[5.5]undecane-3-carboxylate (Intermediate ON)

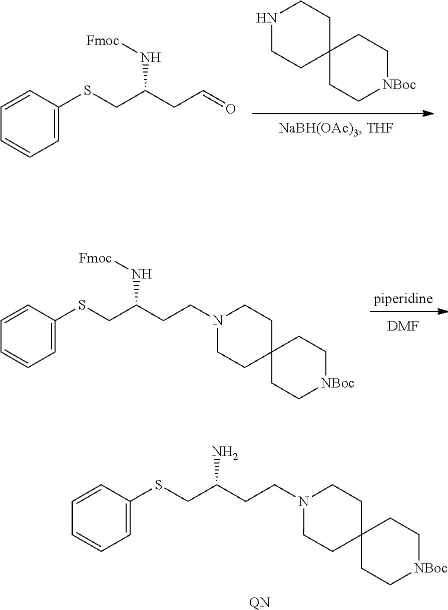

### Step 1—Tert-butyl (R)-9-(3-((((9H-fluoren-9-yl)methoxy) carbonyl)amino)-4-(phenylthio)butyl)-3,9-diazaspiro[5.5]undecane-3-carboxylate

To a solution of (9H-fluoren-9-yl)methyl (R)-(4-oxo-1-(phenylthio)butan-2-yl)carbamate (2 g, 4.79 mmol, synthesized via Steps 1-3 of Intermediate KK) in THF (30 mL) was added tert-butyl 3,9-diazaspiro[5.5]undecane-3-carboxylate (1.57 g, 4.79 mmol, HCl, CAS #173405-78-2) and NaBH(OAc)₃ (1.02 g, 4.79 mmol). The reaction was then stirred at 25° C. for 12 hrs. On completion, the reaction mixture was quenched with water (20 mL) and extracted with ethyl acetate (3×50 mL). The combined organic layers were washed with brine (150 mL) and dried over anhydrous sodium sulfate, filtered and the filtrate was concentrated in vacuo to give the crude residue. The residue was purified by column chromatography (SiO₂, Petroleum ether:Ethyl acetate=1/1 to Dichloromethane:Methanol=20/1) to give the title compound (2 g, 64% yield) as a brown solid. LC-MS (ESI⁺) m/z 656.4. (M+H)⁺.

### Step 2—Tert-butyl (R)-9-(3-amino-4-(phenylthio)butyl)-3,9-diazaspiro[5.5]undecane-3-carboxylate

To a solution of tert-butyl (R)-9-(3-((((9H-fluoren-9-yl)methoxy) carbonyl)amino)-4-(phenylthio)butyl)-3,9-diazaspiro[5.5]undecane-3-carboxylate (2 g, 3.05 mmol) in DMF (10 mL) was added piperidine (260 mg, 3.05 mmol, 301 uL). The reaction was then stirred at 25° C. for 12 hrs. On completion, the reaction mixture was concentrated in vacuo to give the crude residue. The crude residue was purified by reversed-phase HPLC (0.8 g/L ammonium bicarbonate) to give the title compound (600 mg, 40% yield) as a yellow oil. LC-MS (ESI⁺) m/z 434.2. (M+H)⁺.

### Synthesis of Tert-butyl (R)-9-(4-(phenylthio)-3-((4-sulfamoyl-2-((trifluoromethyl)sulfonyl)phenyl)amino)butyl)-3,9-diazaspiro[5.5]undecane-3-carboxylate (Intermediate QO)

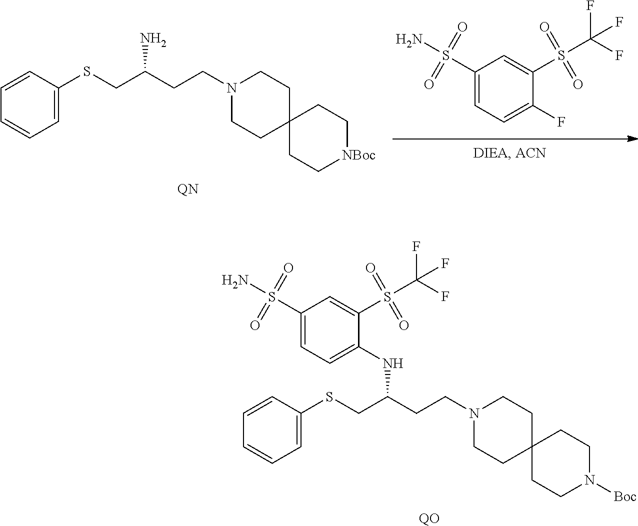

To a solution of tert-butyl (R)-9-(3-amino-4-(phenylthio)butyl)-3,9-diazaspiro[5.5]undecane-3-carboxylate (600 mg, 1.38 mmol, Intermediate QN) and 4-fluoro-3-((trifluoromethyl)sulfonyl)benzenesulfonamide (638 mg, 2.08 mmol, CAS #1027345-08-9) in ACN (10 mL) was added DIEA (1.79 g, 13.8 mmol, 2.41 mL). The reaction was then stirred at 70° C. for 12 hrs. On completion, the reaction mixture was concentrated under reduced pressure to give a residue. The residue was purified by column chromatography (SiO₂, Petroleum ether:Ethyl acetate=1/1 to Dichloromethane: Methanol=20/1) to give the title compound (800 mg, 78% yield) as a brown solid. LC-MS (ESI⁺) m/z 721.3. (M+H)⁺.

### Synthesis of 4-(4-((R)-(4′-chloro-[1,1′-biphenyl]-2-yl)(hydroxy)methyl)piperidin-1-yl)-N-((4-(((R)-1-(phenylthio)-4-(3,9-diazaspiro[5.5]undecan-3-yl)butan-2-yl)amino)-3-((trifluoromethyl)sulfonyl)phenyl)sulfonyl)benzamide (Intermediate QP)

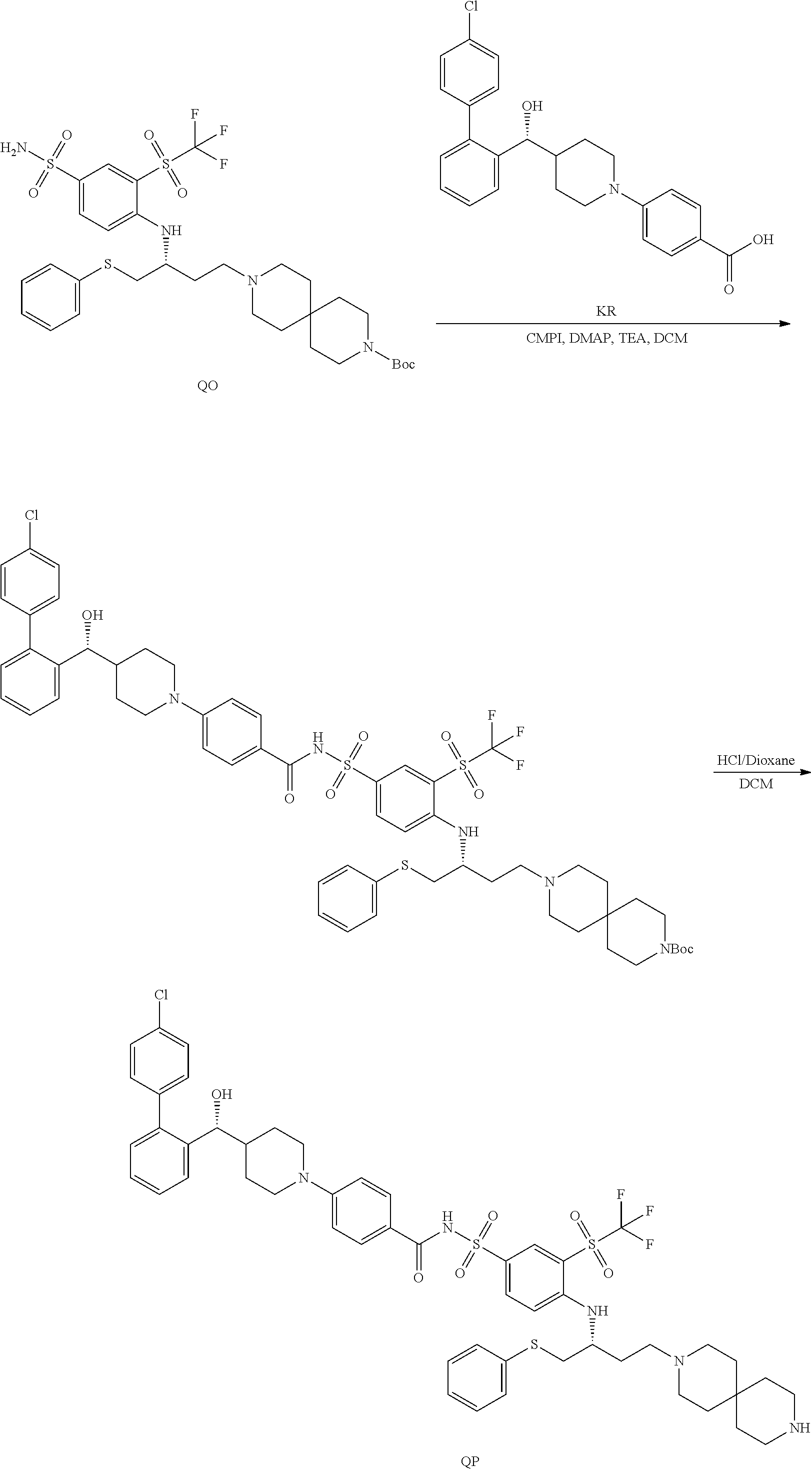

### Step 1—Tert-butyl 9-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)-3,9-diazaspiro[5.5]undecane-3-carboxylate

To a solution of (R)-4-(4-((4′-chloro-[1,1′-biphenyl]-2-yl)(hydroxy)methyl)piperidin-1-yl)benzoic acid (150 mg, 356 μmol, Intermediate KR) in DCM (5 mL) was added DMAP (43.4 mg, 356 μmol), CMPI (136 mg, 533 μmol), TEA (108 mg, 1.07 mmol, 148 uL) and tert-butyl (R)-9-(4-(phenylthio)-3-((4-sulfamoyl-2-((trifluoromethyl)sulfonyl)phenyl)amino)butyl)-3,9-diazaspiro[5.5]undecane-3-carboxylate (308 mg, 427 μmol, Intermediate QO). The reaction was stirred at 25° C. for 2 hrs. On completion, the reaction mixture was concentrated in vacuo to give the crude residue. The crude residue was purified by reversed-phase HPLC (0.1% FA condition) to give the title compound (220 mg, 54% yield) as a white solid. LC-MS (ESI⁺) m/z 563.6. (M+H)⁺.

### Step 2—4-(4-((R)-(4′-chloro-[1,1′-biphenyl]-2-yl)(hydroxy)methyl)piperidin-1-yl)-N-((4-(((R)-1-(phenylthio)-4-(3,9-diazaspiro[5.5]undecan-3-yl)butan-2-yl)amino)-3-((trifluoromethyl)sulfonyl)phenyl)sulfonyl)benzamide

To solution of tert-butyl 9-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)-3,9-diazaspiro[5.5]undecane-3-carboxylate (220 mg, 196 μmol) in DCM (2 mL) was added HCl/dioxane (4 M, 0.5 mL). The reaction was stirred at 25° C. for 1 hr. On completion, the reaction mixture was concentrated in vacuo to give the title compound (200 mg) as a brown solid. LC-MS (ESI+) m/z 512.9. (M+H)⁺.

### Synthesis of 2,2′-(Cyclohexane-1,4-diyl)diacetic Acid (Intermediate QQ)

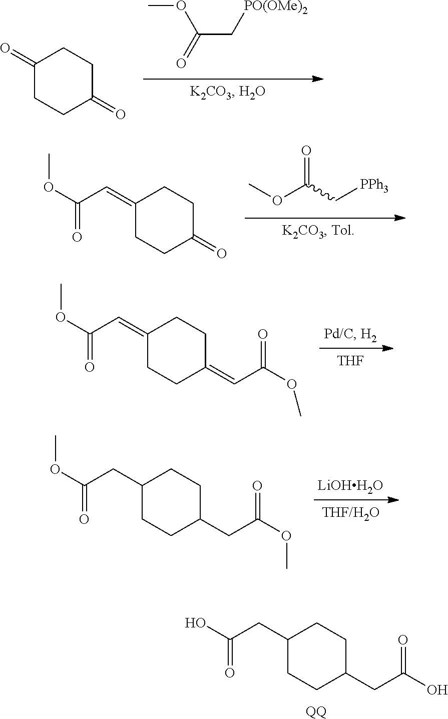

### Step 1—Methyl 2-(4-oxocyclohexylidene)acetate

To a solution of cyclohexane-1,4-dione (10 g, 89.2 mmol, CAS #637-88-7) and methyl 2-(dimethoxyphosphoryl)acetate (40.6 g, 223 mmol, 32.2 mL, CAS #5927-18-4) in H₂O (100 mL) was added K₂CO₃ (24.7 g, 178 mmol). The mixture was then stirred at 50° C. for 16 hrs. On completion, the reaction mixture was quenched with H₂O (50 mL) at 20° C., then diluted with EA (200 mL) and extracted with EA (300 mL×2). The combined organic layers were washed with brine (300 mL×2), dried over Na₂SO₄, filtered and concentrated under reduced pressure to give a residue. The residue was purified by column chromatography (SiO₂, Petroleum ether/Ethyl acetate=10/0 to 5/1) to give the title compound (2 g, 13% yield) as a white oil as a white oil.

### Step 2—Dimethyl 2,2′-(cyclohexane-1,4-diylidene) (2E,2′E)-diacetate

A mixture of methyl 2-(4-oxocyclohexylidene)acetate (2 g, 11.9 mmol), methyl 2-(triphenyl-15-phosphaneylidene)acetate (4.77 g, 14.3 mmol, CAS #2605-67-6), and K₂CO₃ (3.29 g, 23.8 mmol) in toluene (20 mL) was degassed and purged with N₂ three times. Then the mixture was stirred at 60° C. for 16 hrs under N₂ atmosphere. On completion, the reaction mixture was quenched with H₂O (100 mL) at 25° C., and then diluted with EA (100 mL) and extracted with EA (100 mL×2). The combined organic layers were washed with aqueous NaCl (200 mL×2), dried over Na₂SO₄, filtered and concentrated under reduced pressure to give a residue. The residue was purified by column chromatography (SiO₂, Petroleum ether/Ethyl acetate=10/1 to 5/1) to give the title compound (750 mg, 25% yield) as a white solid.

### Step 3—Dimethyl 2,2′-(cyclohexane-1,4-diyl)diacetate

To a solution of dimethyl 2,2′-(cyclohexane-1,4-diylidene) (2E,2′E)-diacetate (90 mg, 401 μmol) in THF (4 mL) was added Pd/C (10 mg, 10% w/w) under N₂ atmosphere. The suspension was degassed and purged with H₂ three times. The mixture was then stirred under H₂ (15 Psi) at 30° C. for 12 hrs. On completion, the reaction was filtered through kieselguhr carefully, and the filtrate was concentrated in vacuo to give a residue. The residue was purified by column chromatography (SiO₂, Petroleum ether/Ethyl acetate=20/1 to 10/1) to give the title compound (80 mg, 79% yield) as a white solid. ¹H NMR (400 MHz, DMSO-d₆) δ=3.57 (s, 6H), 3.29-2.27 (br d, J=7.6 Hz, 1H), 2.18-2.17 (m, 3H), 1.67-1.59 (m, 5H), 1.45-1.40 (m, 1H), 1.35-1.30 (m, 1H), 0.98-0.93 (m, 3H).

### Step 4—2,2′-(Cyclohexane-1,4-diyl)diacetic Acid

To a solution of dimethyl 2,2′-(cyclohexane-1,4-diyl)diacetate (700 mg, 3.07 mmol) in THF (10 mL) and MeOH (2.5 mL) and H₂O (2.5 mL) was added LiOH·H₂O (645 mg, 15.3 mmol). The mixture was stirred at 30° C. for 16 hrs. On completion, the reaction mixture was concentrated under reduced pressure to remove solvent, and the residue was diluted with H₂O (20 mL). The mixture was added HCl (1N) until the pH was 4˜6 and extracted by ethyl acetate (3×50 mL). The combined organic layers were dried over anhydrous sodium sulfate, filtered and the filtrate was concentrated in vacuo to give the title compound (650 mg) as a white solid. ¹H NMR (400 MHz, DMSO-d₆) δ=12.0 (s, 2H), 2.19-2.17 (m, 1H), 2.08-2.06 (m, 3H), 1.68 (s, 1H), 0.97-0.94 (m, 7H), 0.93-0.85 (m, 3H).

### Synthesis of 2-(4-(2-(((S)-1-((2S,4R)-4-hydroxy-2-(((S)-1-(4-(4-methylthiazol-5-yl)phenyl)ethyl) carbamoyl)pyrrolidin-1-yl)-3,3-dimethyl-1-oxobutan-2-yl)amino)-2-oxoethyl)cyclohexyl)acetic Acid (Intermediate QR)

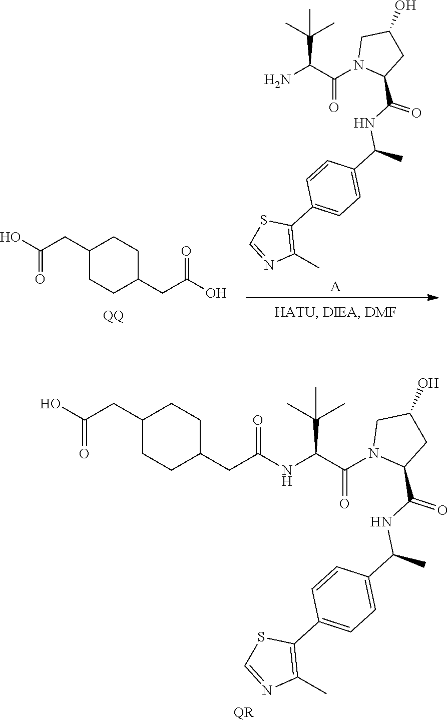

To a solution of 2,2′-(cyclohexane-1,4-diyl)diacetic acid (630 mg, 3.15 mmol, Intermediate QQ) and (2S,4R)-1-[(2S)-2-amino-3,3-dimethyl-butanoyl]-4-hydroxy-N-[(1S)-1-[4-(4-methyl-1,3-thiaphosphol-5-yl)phenyl]ethyl]pyrrolidine-2-carboxamide (1.0 g, 2.17 mmol, Intermediate A) in DMF (1 mL) was added DIEA (840 mg, 6.50 mmol, 1.13 mL). Then HATU (823 mg, 2.17 mmol) was added at 0° C., and the mixture was stirred at 30° C. for 2 hrs. On completion, the reaction mixture was quenched by addition of H₂O (2 mL) at 30° C. The residue was purified by prep-HPLC (FA condition) to give the title compound (2.5 g) as white solid. LC-MS (ESI⁺) m/z 627.5 (M+H)⁺.

### Synthesis of Tert-butyl 4-(chloromethyl)-5-(4-chlorophenyl)-3,6-dihydropyridine-1 (2H)-carboxylate (Intermediate OS)

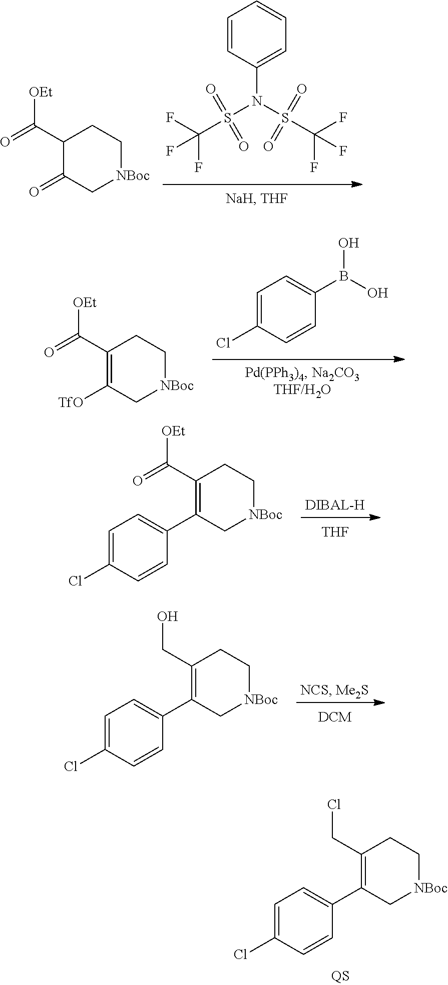

### Step 1—1-(Tert-butyl) 4-ethyl 5-(((trifluoromethyl)sulfonyl)oxy)-3,6-dihydropyridine-1,4 (2H)-dicarboxylate

A solution of 01-tert-butyl 04-ethyl 3-oxopiperidine-1,4-dicarboxylate (5 g, 18.4 mmol, CAS #71223-25-5) in THF (110 mL) was cooled to 0° C., then NaH (958 mg, 24.0 mmol, 60% dispersion in mineral oil) was added at 0° C. under N₂ atmosphere. The mixture was stirred at 0° C. for 0.1 hour, and then 1,1,1-trifluoro-N-phenyl-N-(trifluoromethylsulfonyl)methanesulfonamide (6.91 g, 19.4 mmol, CAS #37595-74-7) was added at 0° C. The resulting mixture was stirred at 25° C. for 20 hrs. On completion, the reaction mixture was quenched with sat, NH₄Cl (50 mL) at 0° C., and then the mixture was diluted with H₂O (30 mL) and extracted with EA (60 mL×3). The combined organic layers were dried over Na₂SO₄, filtered and concentrated under reduced pressure to give a residue. The residue was purified by prep-HPLC (0.1% FA condition) to give the title compound (2.94 g, 39% yield) as a yellow oil. LC-MS (ESI+) m/z 303.9 (M+H)⁺.

### Step 2—1-(Tert-butyl) 4-ethyl 5-(4-chlorophenyl)-3,6-dihydropyridine-1,4 (2H)-dicarboxylate

A mixture of 01-tert-butyl 04-ethyl 5-(trifluoromethylsulfonyloxy)-3,6-dihydro-2H-pyridine-1,4-dicarboxylate (2.94 g, 7.29 mmol), (4-chlorophenyl)boronic acid (1.37 g, 8.75 mmol, CAS #1679-18-1), Na₂CO₃ (2.32 g, 21.9 mmol), Pd(PPh₃)₄ (421.12 mg, 364 μmol) in THF (40 mL) and H₂O (10 mL) was degassed and purged with N₂ three times. Then the mixture was stirred at 65° C. for 3 hr under N₂ atmosphere. On completion, the reaction mixture was diluted with H₂O (30 mL) and extracted with EA (40 mL×3). The combined organic layers were dried over Na₂SO₄, filtered and the filtrate was concentrated under reduced pressure to give a residue. The residue was purified by column chromatography (SiO₂, Petroleum ether/Ethyl acetate=1/0 to 5/1) to give the title compound (2.49 g, 87% yield) as a yellow oil. LC-MS (ESI⁺) m/z 388.0 (M+H)⁺.

### Step 3—Tert-butyl 5-(4-chlorophenyl)-4-(hydroxymethyl)-3,6-dihydropyridine-1 (2H)-carboxylate

To a solution of O1-tert-butyl O4-ethyl 5-(4-chlorophenyl)-3,6-dihydro-2H-pyridine-1,4-dicarboxylate (2.49 g, 6.81 mmol) in THF (30 ml) was added DIBAL-H (1 M, 27.2 ml) under N₂ atmosphere. The mixture was stirred at −78° C. for 3 hrs. On completion, the reaction mixture was quenched with MeOH (5 mL) at 0° C. After warming to 25° C., the mixture was diluted with EA (30 mL) and poured into 40 mL of sat. Rochelle solution, and then extracted with EA (60 mL×3). The combined organic layers were dried over Na₂SO₄, filtered and filtrate was concentrated under reduced pressure to give a residue. The residue was purified by column chromatography (SiO₂, Petroleum ether/Ethyl acetate=5/0 to 3/1) to give the title compound (1.41 g, 64% yield) as a light-yellow oil. LC-MS (ESI⁺) m/z, 346.0 (M+H)⁺; ¹H NMR (400 MHz, DMSO-d₆) δ=7.43 (d, J=8.4 Hz, 2H), 7.26 (d, J=8.4 Hz, 2H), 4.71 (t, J=5.2 Hz, 1H), 3.96 (s, 2H), 3.76 (d, J=5.2 Hz, 2H), 3.49 (t, J=5.2 Hz, 2H), 2.27 (s, 2H), 1.41 (s, 9H).

### Step 4—Tert-butyl 4-(chloromethyl)-5-(4-chlorophenyl)-3,6-dihydropyridine-1 (2H)-carboxylate

To a solution of NCS (1.16 g, 8.71 mmol) in DCM (15 mL) was added Me₂S (595 mg, 9.58 mmol, 703 uL) at 0° C. Then a solution of tert-butyl 5-(4-chlorophenyl)-4-(hydroxymethyl)-3,6-dihydro-2H-pyridine-1-carboxylate (1.41 g, 4.35 mmol) in DCM (15 mL) was added dropwise and the resulting mixture was stirred at 0° C. for 1 hr. On completion, the reaction mixture was quenched with water (100 mL) at 0° C. for 0.5 hrs, then extracted with ethyl acetate (3×100 mL). The combined organic layers were washed with brine (300 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 10/1) to give the title compound (1.24 g, 82% yield) as a yellow oil. LC-MS (ESI⁺) m/z 327.1

### Synthesis of 2-((1H-pyrrolo[2,3-b]pyridin-5-yl)oxy)-4-(4-((1-((1-(tert-butoxycarbonyl)piperidin-4-yl)methyl)-5-(4-chlorophenyl)-1,2,3,6-tetrahydropyridin-4-yl)methyl)piperazin-1-yl)benzoic Acid (Intermediate QT)

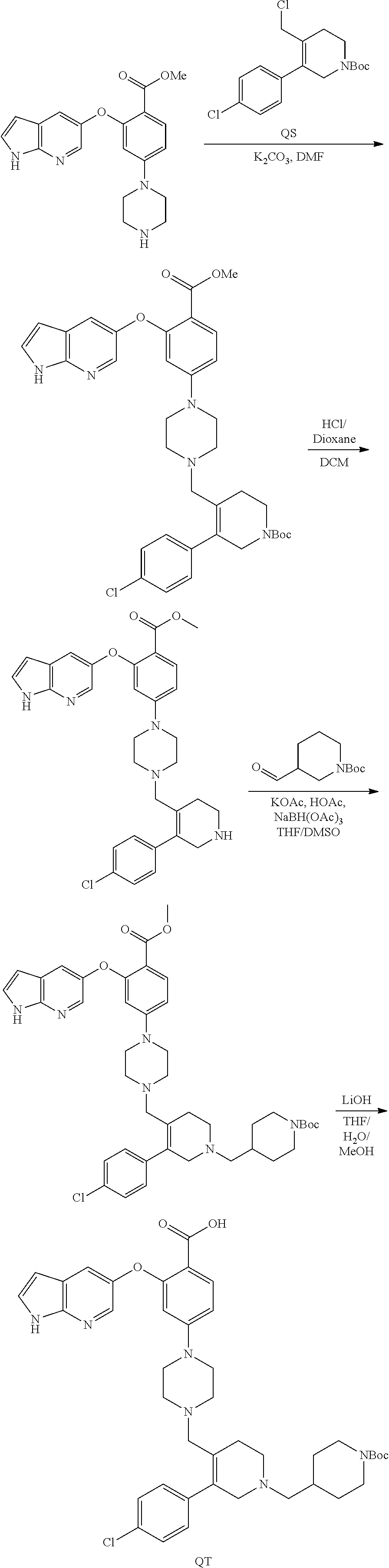

### Step 1—Tert-butyl 4-((4-(3-((1H-pyrrolo[2,3-b]pyridin-5-yl)oxy)-4-(methoxycarbonyl)phenyl)piperazin-1-yl)methyl)-5-(4-chlorophenyl)-3,6-dihydropyridine-1 (2H)-carboxylate

To a solution of tert-butyl 4-(chloromethyl)-5-(4-chlorophenyl)-3,6-dihydro-2H-pyridine-1-carboxylate (1 g, 2.92 mmol, Intermediate QS) and methyl 4-piperazin-1-yl-2-(1H-pyrrolo[2,3-b]pyridin-5-yloxy)benzoate (1.03 g, 2.92 mmol, synthesized via Step 1 of Intermediate NQ) in DMF (2 mL) was added K₂CO₃ (1.01 g, 7.30 mmol). The mixture was stirred at 25° C. for 12 hrs. On completion, the reaction mixture was filtered to give the filtrate. The residue was purified by prep-HPLC (0.1% FA condition) to give a title compound (930 mg, 42% yield, FA) as a yellow solid. LC-MS (ESI⁺) m/z 658.2 (M+H)⁻; ¹H NMR (400 MHz, DMSO-d₆) δ=11.63 (s, 1H), 7.99 (d, J=2.8 Hz, 1H), 7.74 (d, J=9.2 Hz, 1H), 7.48 (t, J=2.8 Hz, 1H), 7.43 (d, J=2.8 Hz, 1H), 7.41 (d, J=8.4 Hz, 2H), 7.17 (d, J=8.4 Hz, 2H), 6.74 (dd, J=2.4, 9.2 Hz, 1H), 6.37 (dd, J=2.0, 3.2 Hz, 1H), 6.32 (d, J=2.4 Hz, 1H), 3.90 (s, 2H), 3.65 (s, 3H), 3.46 (t, J=5.2 Hz, 2H), 3.11 (s, 4H), 2.76 (s, 2H), 2.20 (s, 6H), 1.39 (s, 9H).

### Step 2—Methyl 2-((1H-pyrrolo[2,3-b]pyridin-5-yl)oxy)-4-(4-((5-(4-chlorophenyl)-1,2,3,6-tetrahydropyridin-4-yl)methyl)piperazin-1-yl)benzoate

To a solution of tert-butyl 5-(4-chlorophenyl)-4-[[4-[4-methoxycarbonyl-3-(1H-pyrrolo[2,3-b]pyridin-5-yloxy)phenyl]piperazin-1-yl]methyl]-3,6-dihydro-2H-pyridine-1-carboxylate (830 mg, 1.18 mmol, FA) in DCM (12 mL) was added HCl/dioxane (4 M, 3 mL). The mixture was then stirred at 25° C. for 1 hr. On completion, the reaction mixture was concentrated in vacuo to give the title compound (1 g, HCl) as a gray solid. LC-MS (ESI⁺) m/z 558.2 (M+H)⁺.

### Step 3—Tert-butyl 4-((4-((4-(3-((1H-pyrrolo[2,3-b]pyridin-5-yl)oxy)-4-(methoxycarbonyl)phenyl)piperazin-1-yl)methyl)-5-(4-chlorophenyl)-3,6-dihydropyridin-1(2H)-yl)methyl)piperidine-1-carboxylate

To a solution of methyl 4-[4-[[5-(4-chlorophenyl)-1,2,3,6-tetrahydropyridin-4-yl]methyl]piperazin-1-yl]-2-(1H-pyrrolo[2,3-b]pyridin-5-yloxy)benzoate (500 mg, 841 μmol, HCl) and tert-butyl 4-formylpiperidine-1-carboxylate (269 mg, 1.26 mmol, CAS #137076-22-3) in THF (6 mL) and DMSO (1.5 mL) was added KOAc (248 mg, 2.52 mmol), and HOAc (253 mg, 4.20 mmol) at 25° C. After addition, the mixture was stirred at rt for 1 hr, and then NaBH(OAc)₃ (535 mg, 2.52 mmol) was added at 0° C. The resulting mixture was then stirred at 25° C. for 1 hr. On completion, reaction mixture was concentrated under reduced pressure to remove THF. The mixture was purified by prep-HPLC (0.1% FA condition) to give the title compound as a yellow solid. LC-MS (ESI⁺) m/z 755.2 (M+H)⁺; ¹H NMR (400 MHz, DMSO-d₆) δ=11.62 (s, 1H), 8.15 (s, 1H), 7.99 (d, J=2.8 Hz, 1H), 7.74 (d, J=9.2 Hz, 1H), 7.49-7.45 (m, 1H), 7.42 (d, J=2.4 Hz, 1H), 7.39-7.36 (m, 1H), 7.36-7.34 (m, 1H), 7.15-7.13 (m, 1H), 7.13-7.11 (m, 1H), 6.73 (dd, J=2.4, 9.2 Hz, 1H), 6.37 (dd, J=2.0, 3.2 Hz, 1H), 6.34 (d, J=2.4 Hz, 1H), 3.96-3.85 (m, 4H), 3.64 (s, 3H), 3.23 (d, J=6.4 Hz, 2H), 3.12 (s, 4H), 2.99 (s, 2H), 2.75 (s, 2H), 2.54 (br t, J=5.2 Hz, 2H), 2.21 (br d, J=6.4 Hz, 8H), 1.74-1.57 (m, 5H), 1.38 (s, 9H).

### Step 4—2-((1H-pyrrolo[2,3-b]pyridin-5-yl)oxy)-4-(4-((1-((1-(tert-butoxycarbonyl)piperidin-4-yl)methyl)-5-(4-chlorophenyl)-1,2,3,6-tetrahydropyridin-4-yl)methyl)piperazin-1-yl)benzoic Acid

To a solution of tert-butyl 4-[[5-(4-chlorophenyl)-4-[[4-[4-methoxycarbonyl-3-(1H-pyrrolo[2,3-b]pyridin-5-yloxy)phenyl]piperazin-1-yl]methyl]-3,6-dihydro-2H-pyridin-1-yl]methyl]piperidine-1-carboxylate (556 mg, 736 μmol) in THF (4 mL). MeOH (1 mL) and H₂O (1 mL) was added LiOH·H₂O (154 mg, 3.68 mmol). The mixture was then stirred at 25° C. for 3 hrs. On completion, the reaction mixture was added HCl (1N) until the pH was 7, then the mixture was diluted with water (5 mL) and extracted by ethyl acetate (3×10 mL). The combined organic layers were dried over anhydrous sodium sulfate, filtered and the filtrate was concentrated in vacuo to give the title compound (550 mg) as a yellow solid. LC-MS (ESI⁺) m/z 741.3 (M+H)⁺.

### Synthesis of 2-((1H-pyrrolo[2,3-b]pyridin-5-yl)oxy)-4-(4-((5-(4-chlorophenyl)-1-(piperidin-4-ylmethyl)-1,2,3,6-tetrahydropyridin-4-yl)methyl)piperazin-1-yl)-N-((4-((3-morpholinopropyl)amino)-3-((trifluoromethyl)sulfonyl)phenyl)sulfonyl)benzamide (Intermediate QU)

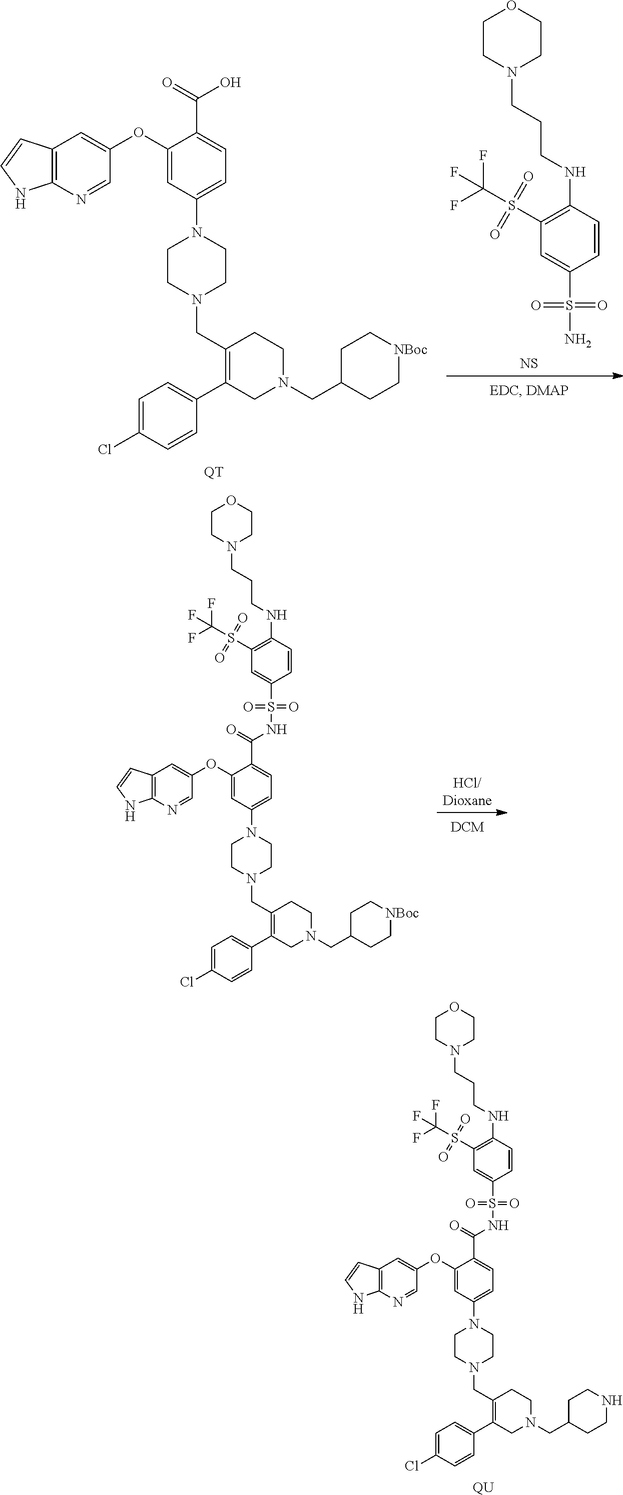

### Step 1—Tert-butyl 4-((4-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-5-(4-chlorophenyl)-3,6-dihydropyridin-1 (2H)-yl)methyl)piperidine-1-carboxylate

To a solution of 4-[4-[[1-[(1-tert-butoxycarbonyl-4-piperidyl)methyl]-5-(4-chlorophenyl)-3,6-dihydro-2H-pyridin-4-yl]methyl]piperazin-1-yl]-2-(1H-pyrrolo[2,3-b]pyridin-5-yloxy)benzoic acid (550 mg, 742 μmol, Intermediate QT) in DCM (10 mL) was added EDC (288 mg, 1.85 mmol), DMAP (227 mg, 1.85 mmol) and 4-(3-morpholinopropylamino)-3-(trifluoromethylsulfonyl)benzenesulfonamide (320 mg, 742 μmol, Intermediate NS). The mixture was stirred at 0° C. for 1 hr. On completion, the reaction mixture was washed with H₂O (3×5 mL) and the organic layer was concentrated in vacuo to give the residue. The residue was purified by prep-HPLC (0.1% EA condition) to give the title compound (542 mg, 60% yield) as a yellow solid. LC-MS (ESI⁺) m/z 1155.6 (M+H)⁺.

### Step 2—2-((1H-pyrrolo[2,3-b]pyridin-5-yl)oxy)-4-(4-((5-(4-chlorophenyl)-1-(piperidin-4-ylmethyl)-1,2,3,6-tetrahydropyridin-4-yl)methyl)piperazin-1-yl)-N-((4-((3-morpholinopropyl)amino)-3-((trifluoromethyl)sulfonyl)phenyl)sulfonyl)benzamide

To a solution of tert-butyl 4-[[5-(4-chlorophenyl)-4-[[4-[4-[[4-(3-morpholinopropylamino)-3-(trifluoromethylsulfonyl)phenyl]sulfonylcarbamoyl]3-(1H-pyrrolo[2,3-b]pyridin-5-yloxy)phenyl]piperazin-1-yl]methyl]-3,6-dihydro-2H-pyridin-1-yl]methyl]piperidine-1-carboxylate (542 mg, 469 μmol) in DCM (8 mL) was added HCl/dioxane (4 M, 2 mL). The mixture was then stirred at 25° C. for 1 hr. On completion, the reaction mixture was concentrated in vacuo to give the title compound (502 mg, HCl) as a yellow solid. LC-MS (ESI⁺) m/z 528.1 (½ M+H)⁺.

### Synthesis of 4-((1-(Tert-butoxycarbonyl)piperidin-4-yl)oxy)butanoic Acid (Intermediate QV)

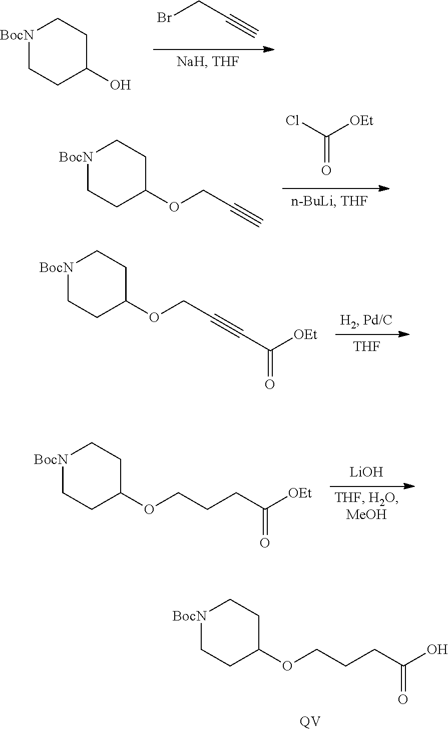

### Step 1—Tert-butyl 4-(prop-2-yn-1-yloxy)piperidine-1-carboxylate

To a solution of tert-butyl 4-hydroxypiperidine-1-carboxylate (3 g, 14.9 mmol, CAS #109384-19-2) in THF (30 mL) was added dropwise NaH (894 mg, 22.4 mmol, 60% dispersion in mineral oil) at 0° C. over 30 mins. After addition, 3-bromoprop-1-yne (1.77 g, 14.9 mmol, 1.28 mL, CAS #106-96-7) in THF (20 mL) was added dropwise at 0° C. The resulting mixture was stirred at 20° C. for 3.5 hrs. On completion, the reaction mixture was quenched with NH₄Cl (50 mL) at 0° C., and then diluted with H₂O (50 mL) and extracted with EA (50 mL×5). The combined organic layers were dried over Na₂SO₄, filtered and concentrated under reduced pressure to give a residue. The residue was purified by column chromatography (SiO₂, Petroleum ether/Ethyl acetate=1/0 to 20/1) to give the title compound (1.92 g, 54% yield) as a yellow oil.

### Step 2—Tert-butyl 4-((4-ethoxy-4-oxobut-2-yn-1-yl)oxy)piperidine-1-carboxylate

A solution of tert-butyl 4-prop-2-ynoxypiperidine-1-carboxylate (500 mg, 2.09 mmol) in THF (10 mL) was cooled to −78° C., then n-BuLi (2.5 M, 919 uL) was added dropwise and stirred at −78° C. for 0.2 hr. The mixture was then stirred at 0° C. for 1.5 hours. Next, ethyl carbonochloridate (345 mg, 3.18 mmol, 302 uL, CAS #541-41-3) was added at −78° C. for 2 hrs. On completion, the reaction mixture was quenched with NaHCO₃ (50 mL) at 0° C., and then diluted with H₂O (10 mL) and extracted with EA (80 mL×3). The combined organic layers were dried over Na₂SO₄ and concentrated under reduced pressure to give the title compound (500 mg) as a yellow oil. LC-MS (ESI⁺) m/z 334.0 (M+Na)⁺.

### Step 3—Tert-butyl 4-(4-ethoxy-4-oxobutoxy)piperidine-1-carboxylate

To a solution of tert-butyl 4-(4-ethoxy-4-oxo-but-2-ynoxy)piperidine-1-carboxylate (1.3 g, 4.18 mmol) in THF (10 mL) was added Pd/C (500 mg, 10 wt %) under Ar. The suspension was degassed under vacuum and purged with H₂ several times. The mixture was stirred under H₂ (15 psi) at 25° C. for 1 hr. On completion, the reaction mixture was filtered carefully with celite. The filtrate was concentrated under reduced pressure to give the title compound (1.22 g) as a yellow oil. LC-MS (ESI⁺) m/z 338.1 (M+H)⁺; ¹H NMR (400 MHz, DMSO-d₆) δ=4.08-4.00 (m, 2H), 3.61-3.54 (m, 2H), 3.40 (t, J=6.4 Hz, 3H), 3.02 (t, J=9.2 Hz, 2H), 2.32 (t, J=7.2 Hz, 2H), 1.77-1.70 (m, 4H), 1.38 (s, 9H), 1.33-1.28 (m, 2H), 1.17 (t, J=7.2 Hz, 3H).

### Step 4—4-((1-(Tert-butoxycarbonyl)piperidin-4-yl)oxy)butanoic Acid

To a solution of tert-butyl 4-(4-ethoxy-4-oxo-butoxy)piperidine-1-carboxylate (1.22 g, 3.87 mmol) in THF (8 mL), H₂O (2 mL) and MeOH (2 mL) was added LiOH·H₂O (812 mg, 19.3 mmol). The mixture was stirred at 25° C. for 2 hrs. On completion, HCl (1N) was added to the reaction mixture until the pH was 7, then diluted by water (5 mL) and extracted by ethyl acetate (3×15 mL). The combined organic layers were dried over anhydrous sodium sulfate, filtered and the filtrate was concentrated in vacuo to give the title compound (1.05 g) as a yellow oil.

### Synthesis of (2S,4R)-1-((S)-3,3-dimethyl-2-(4-(piperidin-4-yloxy)butanamido)butanoyl)-4-hydroxy-N—((S)-1-(4-(4-methylthiazol-5-yl)phenyl)ethyl)pyrrolidine-2-carboxamide (Intermediate QW)

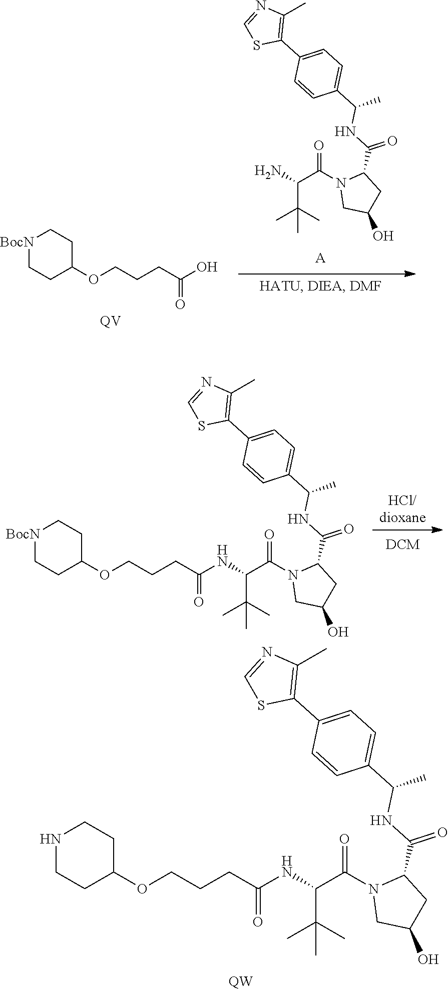

### Step 1—Tert-butyl 4-(4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutoxy)piperidine-1-carboxylate

To a solution of 4-[(1-tert-butoxycarbonyl-4-piperidyl)oxy]butanoic acid (500 mg, 1.74 mmol, Intermediate QV) in DMF (10 mL) was added HATU (860 mg, 2.26 mmol,) and DIEA (1.12 g, 8.70 mmol, 1.52 mL), and then was added (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (774 mg, 1.74 mmol, Intermediate A) at 0° C. The mixture was then stirred at 0° C. for 0.5 hr. On completion, the reaction mixture was filtered to give the filtrate. The filtrate was purified by prep-HPLC (0.1% FA condition) to give the title compound (841 mg, 67% yield) as a yellow solid. LC-MS (ESI⁺) m/z 714.2 (M+H)+.

### Step 2—(2S,4R)-1-((S)-3,3-dimethyl-2-(4-(piperidin-4-yloxy)butanamido)butanoyl)-4-hydroxy-N—((S)-1-(4-(4-methylthiazol-5-yl)phenyl)ethyl)pyrrolidine-2-carboxamide

To a solution of tert-butyl 4-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-4-oxo-butoxyl]piperidine-1-carboxylate (841 mg, 1.18 mmol) in DCM (8 mL) was added HCl/dioxane (4 M, 2 mL). Then the mixture was stirred at 25° C. for 1 hr. On completion, the reaction mixture was concentrated under reduced pressure to give the title compound (850 mg, HCl) as a yellow solid. LC-MS (ESI⁺) m/z 614.2 (M+H)⁺.

### Synthesis of (R)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3,3-diethoxypropyl)amino)-3-((trifluoromethyl)sulfonyl)phenyl)sulfonyl)benzamide (Intermediate QX) and (S)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3,3-diethoxypropyl)amino)-3-((trifluoromethyl)sulfonyl)phenyl)sulfonyl)benzamide (Intermediate QY)

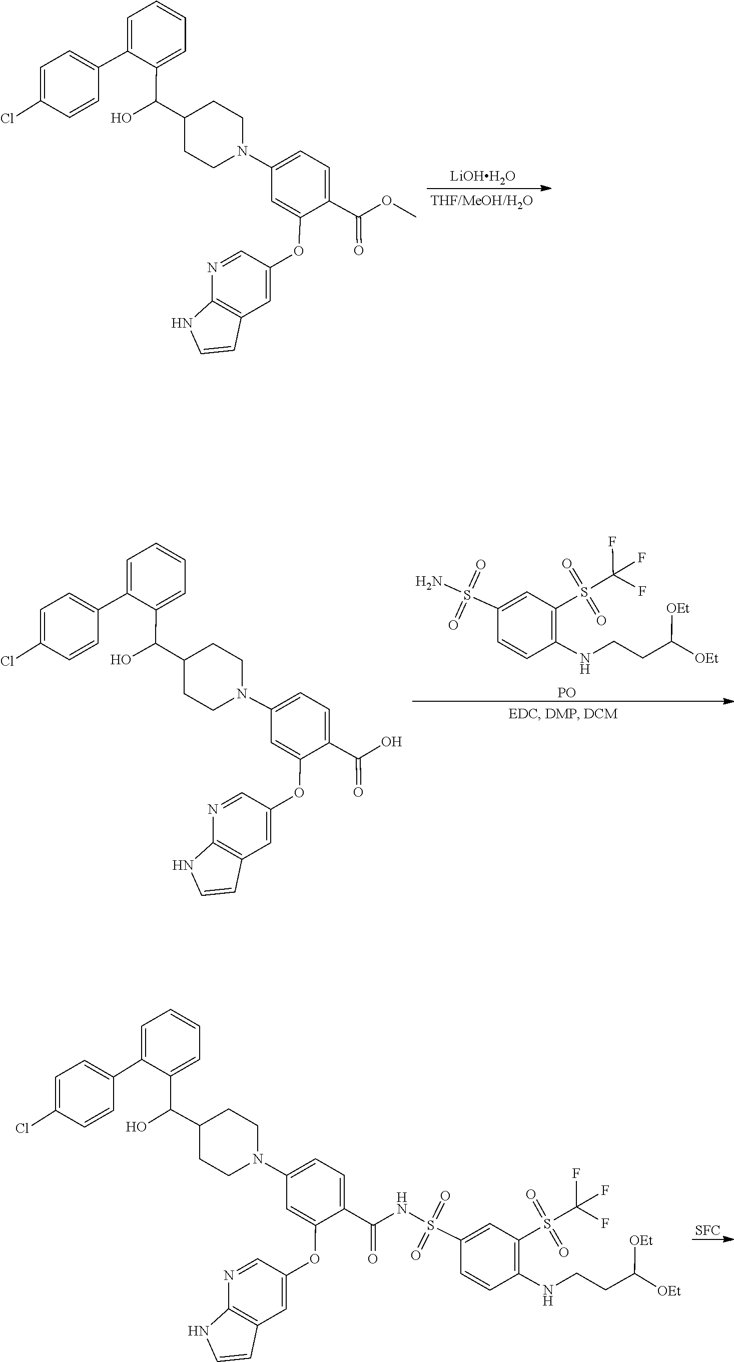

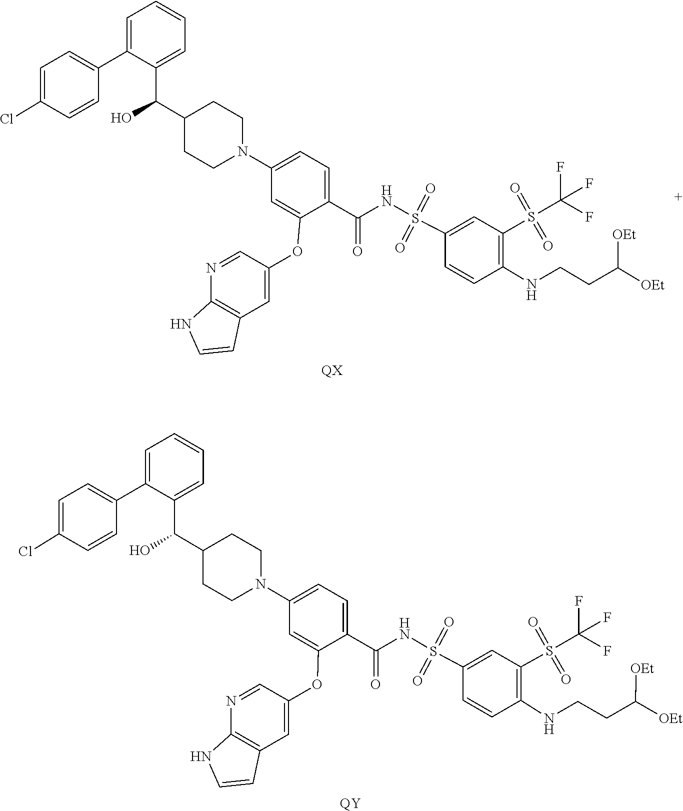

### Step 1—4-[4-[[2-(4-Chlorophenyl)phenyl]-hydroxy-methyl]-1-piperidyl]-2-(1H-pyrrolo[2,3-b]pyridin-5-yloxy)benzoic Acid

To a solution of methyl 2-((1H-pyrrolo[2,3-b]pyridin-5-yl)oxy)-4-(4-((4′-chloro-[1,1′-biphenyl]-2-yl)(hydroxy)methyl)piperidin-1-yl)benzoate (2.8 g, 4.93 mmol, synthesized via Step 1 of Intermediate ME) in THF (8 mL), MeOH (188 mL) and H₂O (4 mL) was added LiOH·H₂O (1.03 g, 24.6 mmol). The mixture was then stirred at 50° C. for 12 hrs. On completion, HCl (1 M) was added to the mixture until the pH 4.0 and the mixture was extracted with DCM (2×60 mL). The combined organic layers were dried over anhydrous sodium sulfate, filtered and the filtrate was concentrated in vacuo to give the title compound (2.6 g) as a white solid. LC-MS (ESI+) m/z 554.1 (M+H)⁺.

### Step 2—2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3,3-diethoxypropyl)amino)-3-((trifluoromethyl)sulfonyl)phenyl)sulfonyl)benzamide

To a solution of 4-[4-[[2-(4-chlorophenyl)phenyl]-hydroxy-methyl]-1-piperidyl]-2-(1H-pyrrolo[2,3-b]pyridin-5-yloxy)benzoic acid (100 mg, 180 μmol) in DCM (2 mL) was added DMAP (55.1 mg, 451 μmol), EDC (56.0 mg, 361 μmol) and 4-(3,3-diethoxypropylamino)-3-(trifluoromethylsulfonyl)benzenesulfonamide (78.4 mg, 181 μmol, Intermediate PO). The mixture was stirred at 30° C. for 3 hrs. On completion, the mixture was concentrated to give a crude product. The residue was purified by prep-HPLC (0.1% FA condition) to give the tittle compound (50 mg, 29% yield) as a white solid. LC-MS (ESI⁺) m/z 970.2 (M+H)⁺.

### Step 3—(R)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3,3-diethoxypropyl)amino)-3-((trifluoromethyl)sulfonyl)phenyl)sulfonyl)benzamide and (S)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3,3-diethoxypropyl)amino)-3-((trifluoromethyl)sulfonyl)phenyl)sulfonyl)benzamide

2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3,3-diethoxypropyl)amino)-3-((trifluoromethyl)sulfonyl)phenyl)sulfonyl)benzamide was purified by SFC (column: DAICEL CHIRALCEL OD (250 mm*30 mm, 10 um); mobile phase: [ACN/MeOH (0.1% NH₃H₂O)]; B %: 40%-40%, A4.5; 160 min) to give (R)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3,3-diethoxypropyl)amino)-3-((trifluoromethyl)sulfonyl)phenyl)sulfonyl)benzamide (1.1 g, 37% yield) as a light yellow solid and (S)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3,3-diethoxypropyl)amino)-3-((trifluoromethyl)sulfonyl)phenyl)sulfonyl)benzamide (400 mg, 13% yield) as a light yellow solid. LC-MS (ESI′) m/z 970.5 (M+H) for both enantiomers. The absolute stereochemistry of the enantiomers was assigned arbitrarily.

### Synthesis of (R)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3-oxopropyl)amino)-3-((trifluoromethyl)sulfonyl)phenyl) sulfonyl)benzamide (Intermediate QZ)

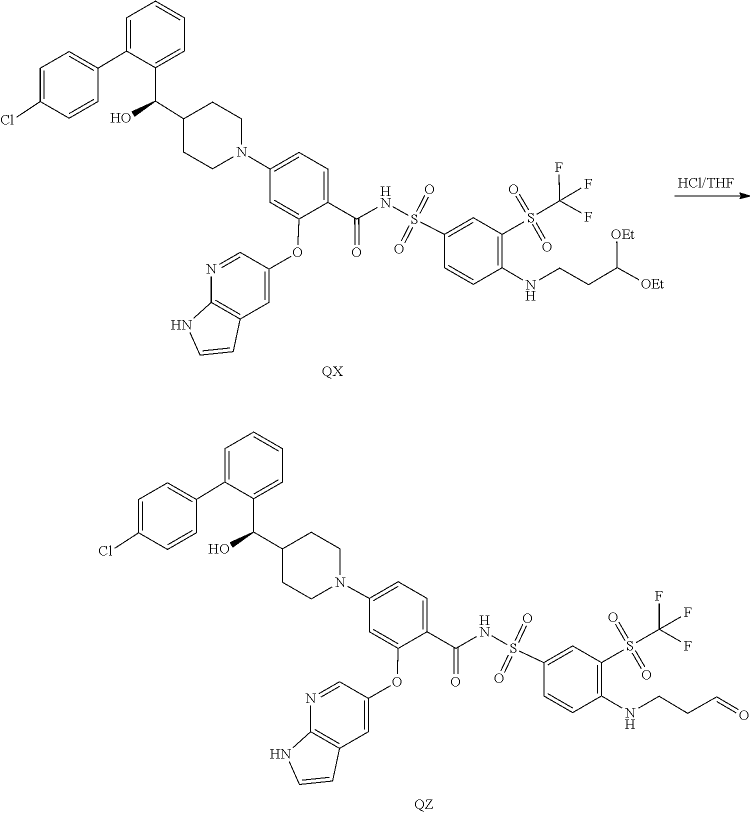

To a solution of 4-[4-[(R)-[2-(4-chlorophenyl)phenyl]-hydroxy-methyl]-1-piperidyl]-N-[4-(3,3-diethoxypropylamino)-3-(trifluoromethylsulfonyl)phenyl]sulfonyl-2-(1H-pyrrolo[2,3-b]pyridin-5-yloxy)benzamide (300 mg, 309 μmol, Intermediate QX) in THF (4 mL) was added HCl (2 M, 155 uL). The mixture was then stirred at 30° C. for 2 hrs. On completion, to the reaction mixture was added sat. NaHCO₃ to adjust the pH to 7, and then extracted with EA (10 mL×3). The combined organic layers were concentrated under reduced pressure to give the title compound (290 mg) as a pink solid. LC-MS (ESI⁺) m/z 896.3 (M+H)⁺.

### Synthesis of (S)-2-((1H-pyrrolo[2,3-b]pyridin-5-yl)oxy)-4-(4-((4′-chloro-[1,1′-biphenyl]-2-yl)(hydroxy)methyl)piperidin-1-yl)-N-((4-((3-oxopropyl)amino)-3-((trifluoromethyl)sulfonyl)phenyl) sulfonyl)benzamide (Intermediate RA)

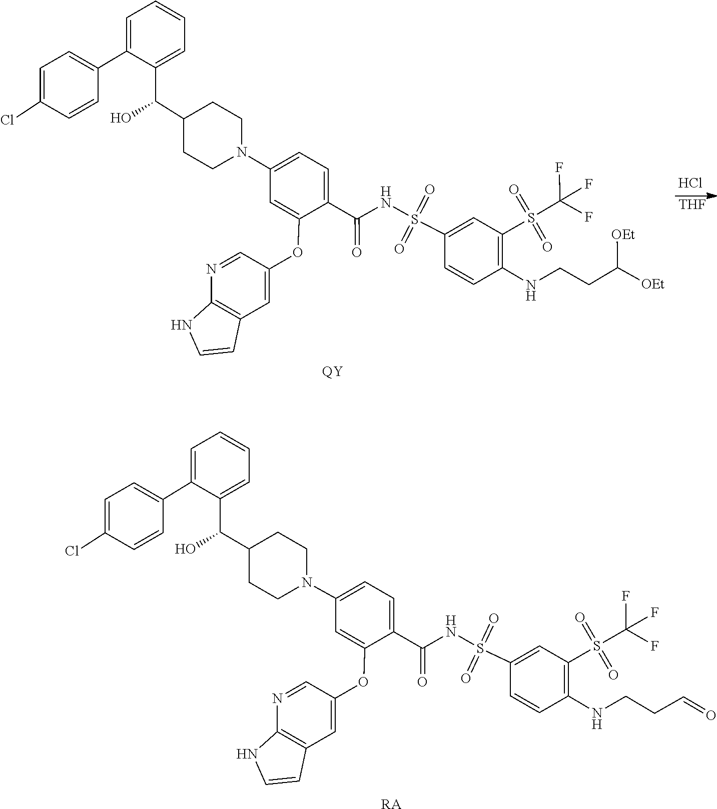

To a solution of 4-[4-[(S)-[2-(4-chlorophenyl)phenyl]-hydroxy-methyl]-1-piperidyl]-N-[4-(3,3-diethoxypropylamino)-3-(trifluoromethylsulfonyl)phenyl]sulfonyl-2-(1H-pyrrolo[2,3-b]pyridin-5-yloxy)benzamide (300 mg, 309.13 μmol, Intermediate QY) in THF (4 mL) was added HCl (2 M, 4 mL). The mixture was stirred at 40° C. for 1 hr. On completion, to the reaction mixture was added sat. NaHCO₃ to adjust the pH to 7, and then extracted with EA (10 mL×3). The combined organic layers were concentrated under reduced pressure to give the title compound (290 mg) as a yellow solid.

### Synthesis of 7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptanoic Acid (Intermediate RB)

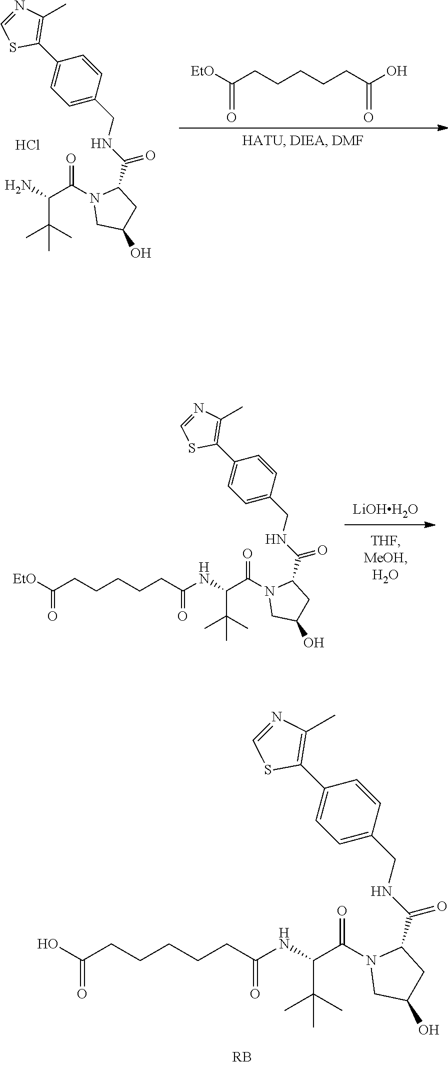 [SMILES: Cc1ncsc1-c1ccc(CNC(=O)[C@@H]2C[C@@H](O)CN2C(=O)[C@@H](N)C(C)(C)C)cc1 ; Cl ; CCOC(=O)CCCCCC(=O)O ; CCOC(=O)CCCCCC(=O)N[C@H](C(=O)N1C[C@H](O)C[C@H]1C(=O)NCc1ccc(-c2scnc2C)cc1)C(C)(C)C ; "LiOH·H2O" ; Cc1ncsc1-c1ccc(CNC(=O)[C@@H]2C[C@@H](O)CN2C(=O)[C@@H](NC(=O)CCCCCC(=O)O)C(C)(C)C)cc1]

### Step 1—Ethyl 7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptanoate

A solution of (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (500 mg, 1.07 mmol, HCl, CAS #1448189-80-7), 7-ethoxy-7-oxoheptanoic acid (302 mg, 1.61 mmol, CAS #33018-91-6), DIEA (692 mg, 5.35 mmol, 932 uL), and HATU (611 mg, 1.61 mmol) in DMF (5 mL) was stirred at 0° C. for 10 min. On completion, the mixture was purified directly by reversed-phase HPLC (0.8 g/L ammonium bicarbonate) to give the title compound (570 mg) as a white solid. ¹H NMR (400 MHz, DMSO-d₆) δ=8.99 (s, 1H), 8.57 (t, J=6.0 Hz, 1H), 7.88 (d, J=9.6 Hz, 1H), 7.40 (q, J=8.4 Hz, 4H), 5.13 (d, J=3.6 Hz, 1H), 4.54 (d, J=9.6 Hz, 1H), 4.48-4.37 (m, 2H), 4.34 (s, 1H), 4.21 (dd, J=5.2, 16.0 Hz, 1H), 4.03 (q, J=7.2 Hz, 2H), 3.74-3.56 (m, 2H), 3.32 (s, 1H), 2.44 (s, 3H), 2.30-2.20 (m, 3H), 2.15-2.06 (m, 1H), 2.06-1.99 (m, 1H), 1.89 (ddd, J=4.4, 8.4, 12.8 Hz, 1H), 1.55-1.41 (m, 4H), 1.30-1.20 (m, 2H), 1.17 (t, J=7.2 Hz, 3H), 0.93 (s, 8H).

### Step 2—7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptanoic Acid

A solution of ethyl 7-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-7-oxoheptanoate (570 mg, 949 μmol) and LiOH·H₂O (199 mg, 4.74 mmol) in THF (2 mL), H₂O (2 mL), and MeOH (2 mL) was stirred at 25° C. for 2 hrs. On completion, the reaction mixture was added HCl (1N) until the pH=7, then diluted by water (10 mL) and extracted by ethyl acetate (10×3 mL). The combined organic layers were dried over anhydrous sodium sulfate, filtered and the filtrate was concentrated in vacuo to give the title compound (510 mg) as a light yellow solid. LC-MS (ESI⁺) m/z 573.5 (M+H)⁺.

### Synthesis of 8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctanoic Acid (Intermediate RC)

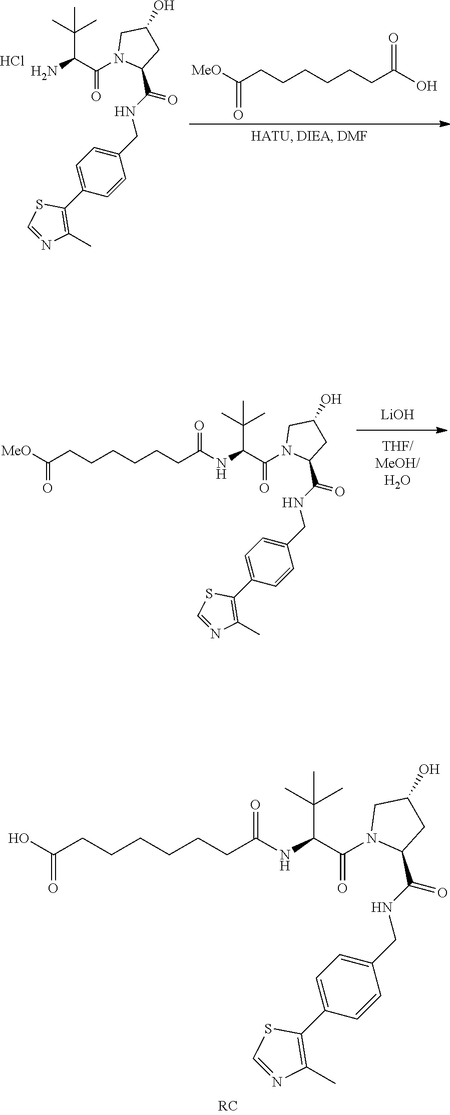 [SMILES: Cl ; Cc1ncsc1-c1ccc(CNC(=O)[C@@H]2C[C@@H](O)CN2C(=O)[C@@H](N)C(C)(C)C)cc1 ; COC(=O)CCCCCCC(=O)O ; COC(=O)CCCCCCC(=O)N[C@H](C(=O)N1C[C@H](O)C[C@H]1C(=O)NCc1ccc(-c2scnc2C)cc1)C(C)(C)C ; "LiOH" ; Cc1ncsc1-c1ccc(CNC(=O)[C@@H]2C[C@@H](O)CN2C(=O)[C@@H](NC(=O)CCCCCCC(=O)O)C(C)(C)C)cc1]

### Step 1—Methyl 8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctanoate

To a solution of 8-methoxy-8-oxo-octanoic acid (197 mg, 1.05 mmol, CAS #3946-32-5) in DMF (5 mL) was added HATU (318 mg, 836.12 μmol), DIEA (450 mg, 3.48 mmol) and (2S,4R)-1-[(2S)-2-amino-3,3-dimethyl-butanoyl]-4-hydroxy-N-[[4-(4-methylthiazol-5-yl)phenyl]methyl]pyrrolidine-2-carboxamide (300 mg, 697 μmol, CAS #1448189-80-7). The mixture was then stirred at 25° C. for 0.5 hrs. On completion, the reaction was concentrated directly and purified by reverse phase (neutral condition) to give the title compound (380 mg, 85% yield) as a white solid. LC-MS (ESI⁺) m/z 601.4 (M+H)⁺.

### Step 2—8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctanoic Acid

To a solution of methyl 8-[[(1S)-1-[(2S,4R)-4-hydroxy-2-[[4-(4-methylthiazol-5-yl)phenyl]methylcarbamoyl]pyrrolidine-1-carbonyl]-2,2-dimethyl-propyl]amino]-8-oxo-octanoate (350 mg, 582 μmol) in THF/MeOH/H₂O (2/2/1, 5 mL) was added LiOH (41.8 mg, 1.75 mmol). The mixture was then stirred at 40° C. for 12 hrs. On completion, the reaction mixture was diluted with water (6 mL) and extracted by ethyl acetate (6×3 mL). The combined organic layers were washed with brine (18 mL) and dried over anhydrous sodium sulfate, filtered and the filtrate was concentrated under vacuum to give the title compound (340 mg) as a white solid. LC-MS (ESI⁺) m/z 587.1 (M+H)⁺.

### Synthesis of (R)-2-((1H-pyrrolo[2,3-b]pyridin-5-yl)oxy)-4-(4-((4-((9-(tert-butoxycarbonyl)-3,9-diazaspiro[5.5]undecan-3-yl)methyl)-4′-chloro-4-methyl-3,4,5,6-tetrahydro-[1,1′-biphenyl]-2-yl)methyl)piperazin-1-yl)benzoic Acid (Intermediate RD)

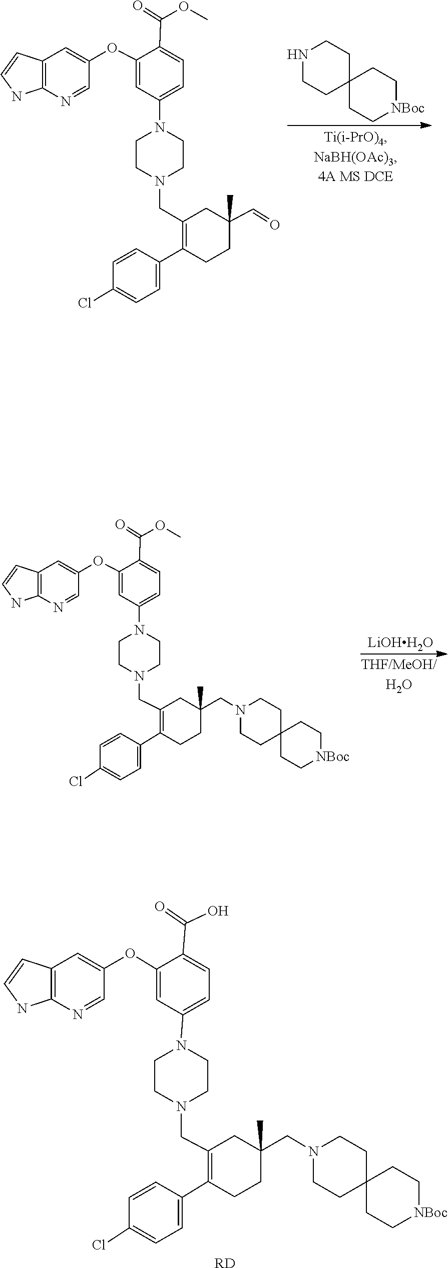

### Step 1—Tert-butyl (R)-9-((6-((4-(3-((1H-pyrrolo[2,3-b]pyridin-5-yl)oxy)-4-(methoxycarbonyl)phenyl)piperazin-1-yl)methyl)-4′-chloro-4-methyl-2,3,4,5-tetrahydro-[1,1′-biphenyl]-4-yl)methyl)-3,9-diazaspiro[5.5]undecane-3-carboxylate

To a solution of methyl 4-[4-[[(5R)-2-(4-chlorophenyl)-5-formyl-5-methyl-cyclohexen-1-yl]methyl]piperazin-1-yl]-2-(1H-pyrrolo[2,3-b]pyridin-5-yloxy)benzoate (300 mg, 500 μmol, synthesized via Steps 1-2 of Intermediate NU), tert-butyl 3,9-diazaspiro[5.5]undecane-3-carboxylate (127 mg, 500 μmol, CAS #173405-78-2) in DCE (5 mL) was added tetraisopropoxytitanium (569 mg, 2.00 mmol) and 4 Å molecular sieves (300 mg, 500 μmol) at 25° C. for 2 h. Next, sodium triacetoxyboranuide (318 mg, 1.50 mmol) was added and the mixture was stirred at 25° C. for 12 hrs. On completion, the reaction mixture was filtered to give a liquid, then the liquid was quenched with NH₄Cl (40 mL), diluted with EA (100 mL) and filtered again, and the mixture was extracted with EA (100 mL). The combined organic layers were dried over anhydrous sodium sulfate and concentrated in vacuo to give the crude residue. The residue was purified by column chromatography (SiO₂, Petroleum ether/Ethyl acetate=1/0 to 0/1, DCM:MeOH=10:1) to give the title compound (90 mg, 21% yield) as a yellow solid. LC-MS (ESI⁺) m/z 837.5 (M+H)⁺.

### Step 2—(R)-2-((1H-pyrrolo[2,3-b]pyridin-5-yl)oxy)-4-(4-((4-((9-(tert-butoxycarbonyl)-3,9-diazaspiro[5.5]undecan-3-yl)methyl)-4′-chloro-4-methyl-3,4,5,6-tetrahydro-[1,1′-biphenyl]-2-yl)methyl)piperazin-1-yl)benzoic Acid

To a solution of tert-butyl 9-[[(1R)-4-(4-chlorophenyl)-3-[[4-[4-methoxycarbonyl-3-(1H-pyrrolo[2,3-b]pyridin-5-yloxy)phenyl]piperazin-1-yl]methyl]-1-methyl-cyclohex-3-en-1-yl]methyl]-3,9-diazaspiro[5.5]undecane-3-carboxylate (90 mg, 107 μmol) in THF (0.5 mL), H₂O (0.5 mL) and MeOH (0.5 mL) was added LiOH·H₂O (45.10 mg, 1.07 mmol). The mixture was stirred at 25° C. for 2 hrs. On completion, the reaction mixture was added HCl (2 M) until the pH 3-5, then the mixture was diluted with water (50 mL) and extracted with dichloromethane (2×100 mL). The combined organic layers were dried over anhydrous sodium sulfate, filtered and the filtrate was concentrated in vacuo to give the title compound (85 mg) as a yellow solid. LC-MS (ESI⁺) m/z 823.5 (M+H)⁺.

### Synthesis of (R)-2-((1H-pyrrolo[2,3-b]pyridin-5-yl)oxy)-4-(4-((4-((3,9-diazaspiro[5,5]undecan-3-yl)methyl)-4′-chloro-4-methyl-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide (Intermediate RE)

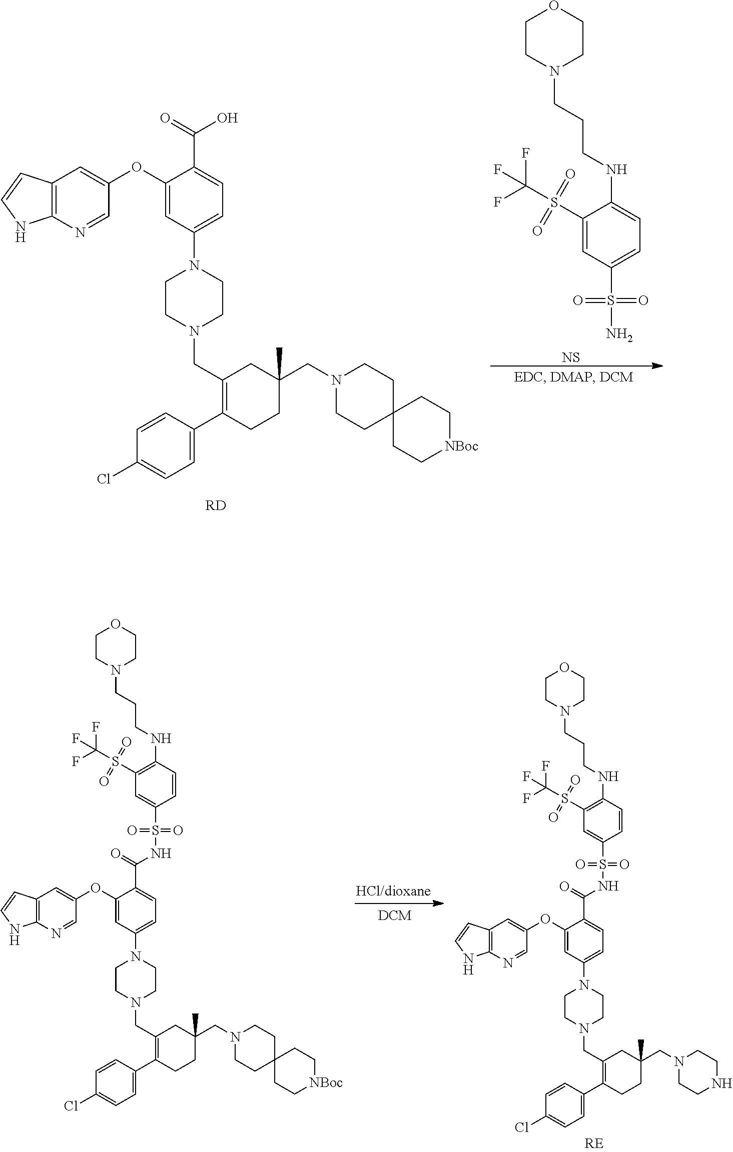 [SMILES: CC(C)(C)OC(=O)N1CCC2(CCN(C[C@]3(C)CCC(c4ccc(Cl)cc4)=C(CN4CCN(c5ccc(C(=O)O)c(Oc6cnc7[nH]ccc7c6)c5)CC4)C3)CC2)CC1 ; NS(=O)(=O)c1ccc(NCCCN2CCOCC2)c(S(=O)(=O)C(F)(F)F)c1 ; "NS" ; "RD" ; CC(C)(C)OC(=O)N1CCC2(CCN(C[C@]3(C)CCC(c4ccc(Cl)cc4)=C(CN4CCN(c5ccc(C(=O)NS(=O)(=O)c6ccc(NCCCN7CCOCC7)c(S(=O)(=O)C(F)(F)F)c6)c(Oc6cnc7[nH]ccc7c6)c5)CC4)C3)CC2)CC1 ; C[C@@]1(CN2CCNCC2)CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1]

### Step 1—Tert-butyl (R)-9-((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-3,9-diazaspiro[5.5]undecane-3-carboxylate

To a solution of 4-[4-[[(5R)-5-[(3-tert-butoxycarbonyl-3,9-diazaspiro[5.5]undecan-9-yl)methyl]-2-(4-chlorophenyl)-5-methyl-cyclohexen-1-yl]methyl]piperazin-1-yl]-2-(1H-pyrrolo[2,3-b]pyridin-5-yloxy)benzoic acid (80 mg, 97 μmol, Intermediate RD) and 4-(3-morpholinopropylamino)-3-(trifluoromethylsulfonyl)benzenesulfonamide (41.9 mg, 97.1 μmol, Intermediate NS) in DCM (1 mL) was added EDC (37.70 mg, 242 μmol) and DMAP (29.6 mg, 242 μmol). The mixture was then stirred at 25° C. for 3 hrs. On completion, the reaction mixture was filtered and the filtrate was concentrated in vacuo to give the crude residue. The residue was purified by prep-HPLC (basic condition) to give a compound (40 mg, 33% yield) as a white solid. LC-MS (ESI⁺) m/z 1237.7 (M+H)⁺.

### Step 2 (R)-2-((1H-pyrrolo[2,3-b]pyridin-5-yl)oxy)-4-(4-((4-((3,9-diazaspiro[5.5]undecan-3-yl)methyl)-4′-chloro-4-methyl-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide

To a solution of tert-butyl 9-[[(1R)-4-(4-chlorophenyl)-1-methyl-3-[[4-[4-[[4-(3-morpholinopropylamino)-3-(trifluoromethylsulfonyl)phenyl]sulfonylcarbamoyl]-3-(1H-pyrrolo[2,3-b]pyridin-5-yloxy)phenyl]piperazin-1-yl]methyl]cyclohex-3-en-1-yl]methyl]-3,9-diazaspiro[5.5]undecane-3-carboxylate (40.9 mg, 33.0 μmol) in DCM (2 mL) was added HCl/dioxane (4 M, 8.27 uL). The mixture was then stirred at 25° C. for 2 hrs. On completion, the reaction mixture was concentrated under reduced pressure to give the title compound (40 mg, HCl) as a white solid.

### Synthesis of (R)-2-((1H-pyrrolo[2,3-b]pyridin-5-yl)oxy)-4-(4-((4-(4-(tert-butoxycarbonyl)piperazine-1-carbonyl)-4′-chloro-4-methyl-3,4,5,6-tetrahydro-[1,1′-biphenyl]-2-yl)methyl)piperazin-1-yl)benzoic Acid (Intermediate RF)

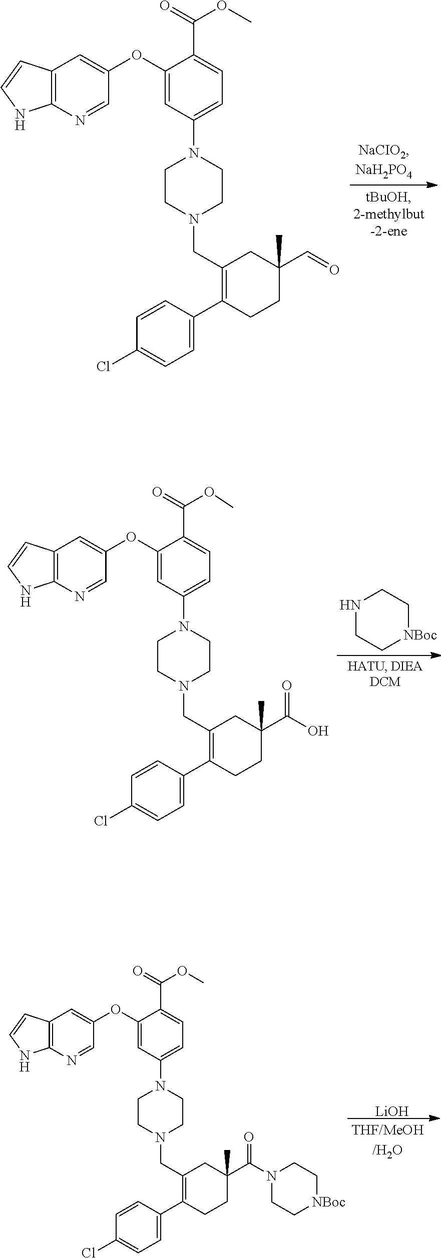 [SMILES: COC(=O)c1ccc(N2CCN(CC3=C(c4ccc(Cl)cc4)CC[C@@](C)(C=O)C3)CC2)cc1Oc1cnc2[nH]ccc2c1 ; COC(=O)c1ccc(N2CCN(CC3=C(c4ccc(Cl)cc4)CC[C@@](C)(C(=O)O)C3)CC2)cc1Oc1cnc2[nH]ccc2c1 ; CC(C)(C)OC(=O)N1CCNCC1 ; COC(=O)c1ccc(N2CCN(CC3=C(c4ccc(Cl)cc4)CC[C@@](C)(C(=O)N4CCN(C(=O)OC(C)(C)C)CC4)C3)CC2)cc1Oc1cnc2[nH]ccc2c1 ; "LiOH"]

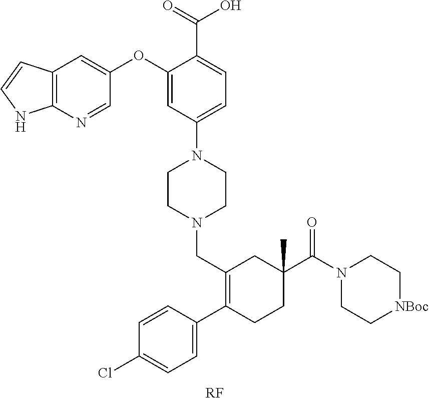

### Step 1—(R)-6-((4-(3-((1H-pyrrolo[2,3-b]pyridin-5-yl)oxy)-4-(methoxycarbonyl)phenyl)piperazin-1-yl)methyl)-4′-chloro-4-methyl-2,3,4,5-tetrahydro-[1,1′-biphenyl]-4-carboxylic Acid

To a solution of methyl 4-[4-[[(5R)-2-(4-chlorophenyl)-5-formyl-5-methyl-cyclohexen-1-yl]methyl]piperazin-1-yl]-2-(1H-pyrrolo[2,3-b]pyridin-5-yloxy)benzoate (400 mg, 668 μmol, synthesized via Steps 1-2 of Intermediate NU) in t-butanol (8 mL) was added NaH₂PO₄ (336 mg, 2.80 mmol) and 2-methyl-2-butene (2.0 mL). The solution was then cooled to 0° C. A freshly prepared solution of sodium hypochlorite (NaClO₂, 20% in water, 0.8 mL) was added to the mixture. The resulting solution was stirred for 2 hrs at 0° C. On completion, the reaction mixture was quenched with water (8 mL) and extracted with ethyl acetate (30 mL). The combined organic layers were washed with brine (10 mL) and dried over anhydrous sodium sulfate, filtered and the filtrate was concentrated in vacuo to give the a residue. The residue was purified by reversed-phase HPLC (0.1% FA condition) to give the title compound (200 mg, 45% yield, FA) as a yellow solid. LC-MS (ESI⁺) m/z 615.3 (M+H)⁺.

### Step 2—Tert-butyl (R)-4-(6-((4-(3-((1H-pyrrolo[2,3-b]pyridin-5-yl)oxy)-4-(methoxycarbonyl)phenyl)piperazin-1-yl)methyl)-4′-chloro-4-methyl-2,3,4,5-tetrahydro-[1,1′-biphenyl]-4-carbonyl)piperazine-1-carboxylate

To a solution of (R)-6-((4-(3-((1H-pyrrolo[2,3-b]pyridin-5-yl)oxy)-4-(methoxycarbonyl)phenyl)piperazin-1-yl)methyl)-4′-chloro-4-methyl-2,3,4,5-tetrahydro-[1,1′-biphenyl]-4-carboxylic acid (150 mg, 244 μmol) in DMF (5 mL) was added DIEA (158 mg, 1.22 mmol), HATU (139 mg, 366 μmol), and tert-butyl piperazine-1-carboxylate (54.5 mg, 293 μmol, CAS #57260-71-6). The mixture was then stirred at 25° C. for 2 hrs. On completion, the reaction mixture was quenched with water (5 mL) and extracted with DCM (30 mL). The combined organic layers were washed with brine (30 mL) and dried over anhydrous sodium sulfate, filtered and the filtrate was concentrated in vacuo to give the residue. The residue was purified by reversed-phase HPLC (0.1% FA condition) to give the title compound (175 mg, 78% yield, FA) as a white solid. LC-MS (ESI⁺) m/z 783.2 (M+H)⁺.

### Step 3—(R)-2-((1H-pyrrolo[2,3-b]pyridin-5-yl)oxy)-4-(4-((4-(4-(tert-butoxycarbonyl)piperazine-1-carbonyl)-4′-chloro-4-methyl-3,4,5,6-tetrahydro-[1,1′-biphenyl]-2-yl)methyl)piperazin-1-yl)benzoic Acid

To a solution of tert-butyl 4-[(1R)-4-(4-chlorophenyl)-3-[[4-[4-methoxycarbonyl-3-(1H-pyrrolo[2,3-b]pyridin-5-yloxy)phenyl]piperazin-1-yl]methyl]-1-methyl-cyclohex-3-ene-1-carbonyl]piperazine-1-carboxylate (175 mg, 223 μmol) in H₂O (1.5 mL), THF (3 mL) and MeOH (3 mL), then LiOH·H₂O (46.9 mg, 1.12 mmol) was added. The mixture was then stirred at 50° C. for 12 hrs. On completion, the mixture was adjusted pH to 6 by adding 1M HCl solution, diluted with H₂O (10 mL), and extracted with DCM/THF (3:1, 30 mL×3). The combined organic layer was dried over anhydrous Na₂SO₄, filtered and the filtrate was concentrated to give the title compound (190 mg) as a white solid. LC-MS (ESI⁺) m/z 769.4 (M+H)⁺.

### Synthesis of (R)-2-((1H-pyrrolo[2,3-b]pyridin-5-yl)oxy)-4-(4-((4′-chloro-4-methyl-4-(piperazine-1-carbonyl)-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide (Intermediate RG)

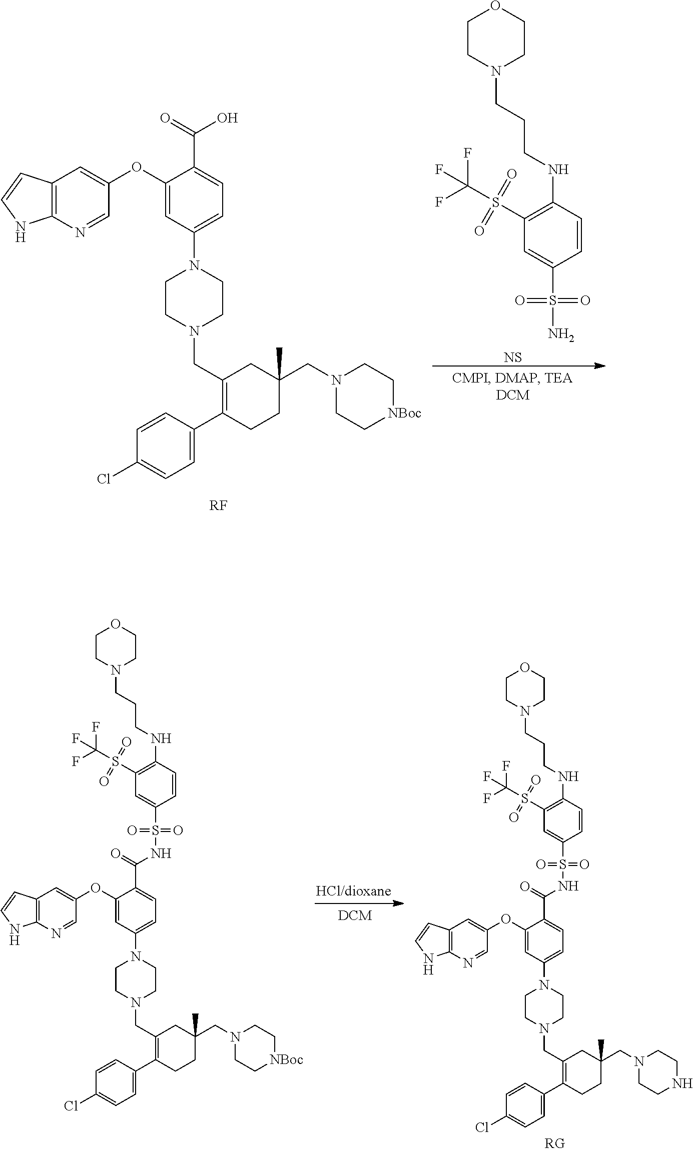 [SMILES: CC(C)(C)OC(=O)N1CCN(C[C@]2(C)CCC(c3ccc(Cl)cc3)=C(CN3CCN(c4ccc(C(=O)O)c(Oc5cnc6[nH]ccc6c5)c4)CC3)C2)CC1 ; NS(=O)(=O)c1ccc(NCCCN2CCOCC2)c(S(=O)(=O)C(F)(F)F)c1 ; "NS" ; "RF" ; CC(C)(C)OC(=O)N1CCN(C[C@]2(C)CCC(c3ccc(Cl)cc3)=C(CN3CCN(c4ccc(C(=O)NS(=O)(=O)c5ccc(NCCCN6CCOCC6)c(S(=O)(=O)C(F)(F)F)c5)c(Oc5cnc6[nH]ccc6c5)c4)CC3)C2)CC1 ; C[C@@]1(CN2CCNCC2)CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1]

### Step 1—Tert-butyl (R)-4-(6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-carbonyl)piperazine-1-carboxylate

To a solution of 4-[4-[[(5R)-5-(4-tert-butoxycarbonylpiperazine-1-carbonyl)-2-(4-chlorophenyl)-5-methylcyclohexen-1-yl]methyl]piperazin-1-yl]-2-(1H-pyrrolo[2,3-b]pyridin-5-yloxy)benzoic acid (190 mg, 247 μmol, Intermediate RF) in DCM (4 mL) was added DMAP (30.2 mg, 247 μmol), TEA (37.5 mg, 370 μmol) and CMPI (94.6 mg, 370 μmol). Then 4-(3-morpholinopropylamino)-3-(trifluoromethylsulfonyl)benzenesulfonamide (107 mg, 247 μmol, Intermediate NS) was added and the mixture was stirred at 25° C. for 1 hr. On completion, the mixture was concentrated to give a crude product. The crude product was purified by reversed-phase HPLC (0.1% FA condition) to give the title compound (170 mg, 55% yield, 98% purity) as a white solid. LC-MS (ESI⁺) m/z 1182.8 (M+H)⁺.

### Step 2—(R)-2-((1H-pyrrolo[2,3-b]pyridin-5-yl)oxy)-4-(4-((4′-chloro-4-methyl-4-(piperazine-1-carbonyl)-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide

To a solution of tert-butyl 4-[(1R)-4-(4-chlorophenyl)-1-methyl-3-[[4-[4-[[4-(3-morpholinopropylamino)-3-(trifluoromethylsulfonyl)phenyl]sulfonylcarbamoyl]-3-(1H-pyrrolo[2,3-b]pyridin-5-yloxy)phenyl]piperazin-1-yl]methyl]cyclohex-3-ene-1-carbonyl]piperazine-1-carboxylate (170 mg, 138 μmol) in DCM (10 mL) was added HCl/dioxane (4 M, 1.55 mL). The mixture was stirred at 20° C. for 1 hr. On completion, the mixture was concentrated to give the title product as a white solid. LC-MS (ESI⁺) m/z 1082.2 (M+H)⁺.

### Synthesis of (R)-2-((1H-pyrrolo[2,3-b]pyridin-5-yl)oxy)-4-(4-((4-((2-(tert-butoxycarbonyl)-2,7-diazaspiro[3.5]nonan-7-yl)methyl)-4′-chloro-4-methyl-3,4,5,6-tetrahydro-[1,1′-biphenyl]-2-yl)methyl)piperazin-1-yl)benzoic Acid (Intermediate RH)

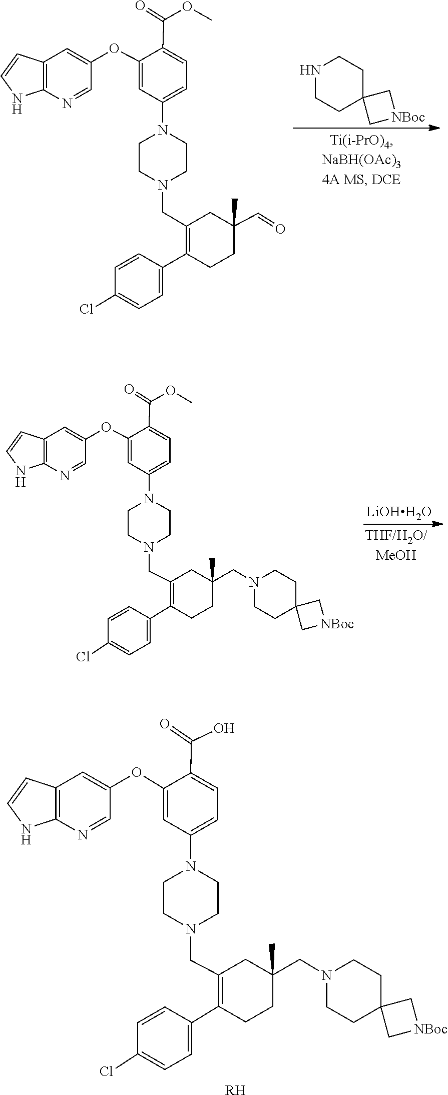 [SMILES: COC(=O)c1ccc(N2CCN(CC3=C(c4ccc(Cl)cc4)CC[C@@](C)(C=O)C3)CC2)cc1Oc1cnc2[nH]ccc2c1 ; CC(C)(C)OC(=O)N1CC2(CCNCC2)C1 ; COC(=O)c1ccc(N2CCN(CC3=C(c4ccc(Cl)cc4)CC[C@@](C)(CN4CCC5(CC4)CN(C(=O)OC(C)(C)C)C5)C3)CC2)cc1Oc1cnc2[nH]ccc2c1 ; "LiOH·H2O" ; CC(C)(C)OC(=O)N1CC2(CCN(C[C@]3(C)CCC(c4ccc(Cl)cc4)=C(CN4CCN(c5ccc(C(=O)O)c(Oc6cnc7[nH]ccc7c6)c5)CC4)C3)CC2)C1 ; "RH"]

### Step 1—Tert-butyl (R)-2-((6-((4-(3-((1H-pyrrolo[2,3-b]pyridin-5-yl)oxy)-4-(methoxycarbonyl)phenyl)piperazin-1-yl)methyl)-4′-chloro-4-methyl-2,3,4,5-tetrahydro-[1,1′-biphenyl]-4-yl)methyl)-2,7-diazaspiro[3.5]nonane-7-carboxylate

To a solution of methyl 4-[4-[[(5R)-2-(4-chlorophenyl)-5-formyl-5-methyl-cyclohexen-1-yl]methyl]piperazin-1-yl]-2-(1H-pyrrolo[2,3-b]pyridin-5-yloxy)benzoate (300 mg, 500.74 μmol, synthesized via Steps 1-2 of Intermediate NU), tert-butyl 2,7-diazaspiro[3.5]nonane-2-carboxylate (113 mg, 500 μmol, CAS #236406-55-6), tetraisopropoxytitanium (569 mg, 2.00 mmol) and 4 Å molecular sieves (300 mg, 500.74 μmol) in DCE (5 mL) was stirred at 25° C. for 2 hrs. Then sodium triacetoxyboranuide (318 mg, 1.50 mmol) was added at 0° C. and the mixture was stirred at 25° C. for 12 hrs. On completion, the reaction mixture was filtered to give a liquid, then the liquid was quenched with NH₄Cl (40 mL), diluted with EA (100 mL) and filtered again, then extracted with EA (100 mL). The organic layer was dried over anhydrous sodium sulfate and concentrated in vacuo to give the crude residue. The residue was purified by column chromatography (SiO₂, Petroleum ether/Ethyl acetate=1/0 to 0/1) to give the title compound (130 mg, 32% yield) as a yellow solid. LC-MS (ESI⁺) m/z 809.5 (M+H)⁺.

### Step 2—(R)-2-((1H-pyrrolo[2,3-b]pyridin-5-yl)oxy)-4-(4-((4-((2-(tert-butoxycarbonyl)-2,7-diazaspiro[3.5]nonan-7-yl)methyl)-4′-chloro-4-methyl-3,4,5,6-tetrahydro-[1,1′-biphenyl]-2-yl)methyl)piperazin-1-yl)benzoic Acid

To a solution of tert-butyl 7-[[(1R)-4-(4-chlorophenyl)-3-[[4-[4-methoxycarbonyl-3-(1H-pyrrolo[2,3-b]pyridin-5-yloxy)phenyl]piperazin-1-yl]methyl]-1-methyl-cyclohex-3-en-1-yl]methyl]-2,7-diazaspiro[3.5]nonane-2-carboxylate (120 mg, 148 μmol) in THF (1 mL). MeOH (1 mL) and H₂O (1 mL) was added LiOH·H₂O (31.1 mg, 741 μmol). The mixture was then stirred at 25° C. for 14 hrs. On completion, HCl (2 M) was added to the reaction mixture until pH 3-5, then diluted with water (50 mL) and extracted by dichloromethane (100×2 mL). The combined organic layers were dried over anhydrous sodium sulfate, filtered and the filtrate was concentrated in vacuo to give the title compound (120 mg) as a white solid. LC-MS (ESI⁺) m/z 795.2 (M+H)⁺.

### Synthesis of (R)-2-((1H-pyrrolo[2,3-b]pyridin-5-yl)oxy)-4-(4-((4-((2,7-diazaspiro[3.5]nonan-7-yl)methyl)-4′-chloro-4-methyl-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide (Intermediate RI)

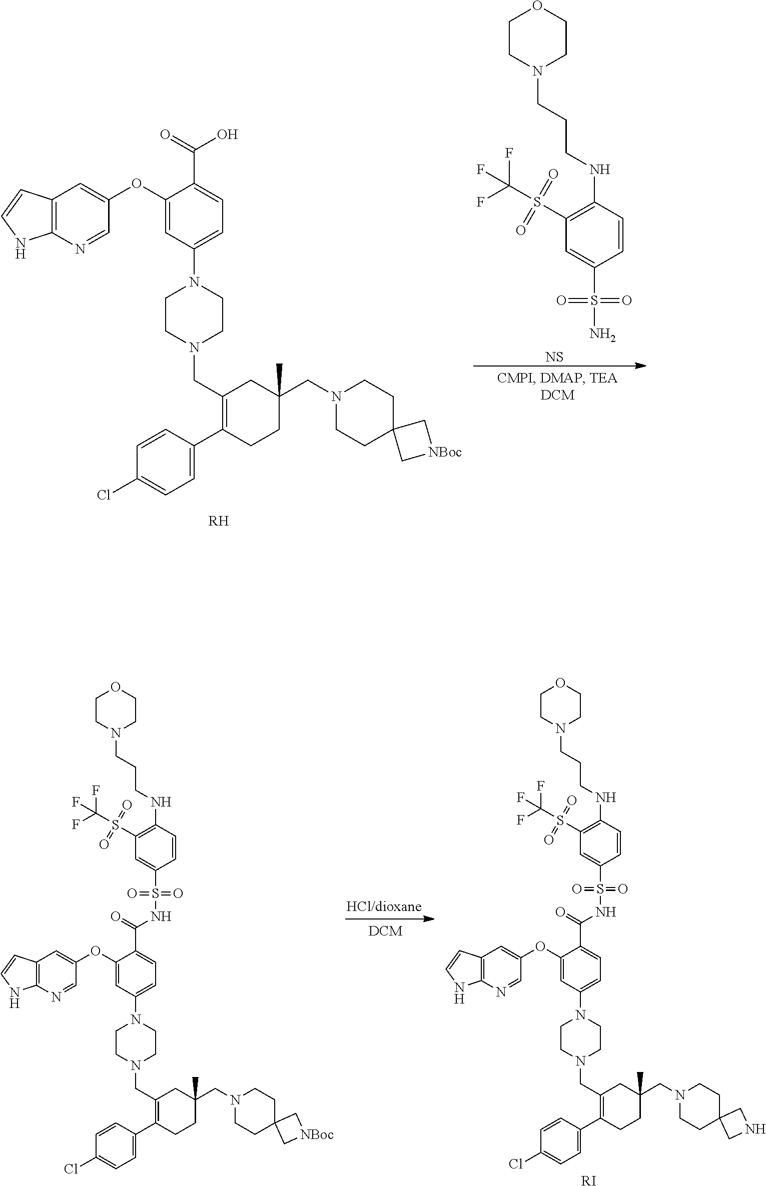

### Step 1—Tert-butyl (R)-7-((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-2,7-diazaspiro[3.5]nonane-2-carboxylate

To a solution of 4-[4-[[(5R)-5-[(2-tert-butoxycarbonyl-2,7-diazaspiro[3.5]nonan-7-yl)methyl]-2-(4-chlorophenyl)-5-methyl-cyclohexen-1-yl]methyl]piperazin-1-yl]-2-(1H-pyrrolo[2,3-b]pyridin-5-yloxy)benzoic acid (105 mg, 132 μmol, Intermediate RH) and 4-(3-morpholinopropylamino)-3-(trifluoromethylsulfonyl)benzenesulfonamide (56 mg, 132 μmol, Intermediate NS) in DCM (2 mL) was added DMAP (40.3 mg, 330 μmol) and EDC (51.2 mg, 330 μmol). The mixture was then stirred at 0° C. for 2 hrs. On completion, the reaction mixture was filtered and the filtrate was concentrated in vacuo to give the crude residue. The residue was purified by prep-HPLC (basic condition) to give a compound (90 mg, 55% yield) as a white solid. LC-MS (ESI⁺) m/z 1208.2 (M+H)⁺.

### Step 2—(R)-2-((1H-pyrrolo[2,3-b]pyridin-5-yl)oxy)-4-(4-((4-((2,7-diazaspiro[3.5]nonan-7-yl)methyl)-4′-chloro-4-methyl-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide

To a solution of tert-butyl 7-[[(1R)-4-(4-chlorophenyl)-1-methyl-3-[[4-[4-[[4-(3-morpholinopropylamino)-3-(trifluoromethylsulfonyl)phenyl]sulfonylcarbamoyl]-3-(1H-pyrrolo[2,3-b]pyridin-5-yloxy)phenyl]piperazin-1-yl]methyl]cyclohex-3-en-1-yl]methyl]-2,7-diazaspiro[3.5]nonane-2-carboxylate (80.0 mg, 66.1 μmol) in DCM (2 mL) was added TFA (3.08 g, 27.0 mmol). The mixture was then stirred at 25° C. for 2 hrs. On completion, the reaction mixture was filtered and the filtrate was concentrated in vacuo to give the crude residue. The residue was purified by prep-HPLC (neutral condition) to give a compound (50 mg, 68% yield) as a white solid. LC-MS (ESI⁺) m/z 1108.4 (M+H)⁺.

### Synthesis of 8-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-8-oxooctanoic Acid (Intermediate RJ)

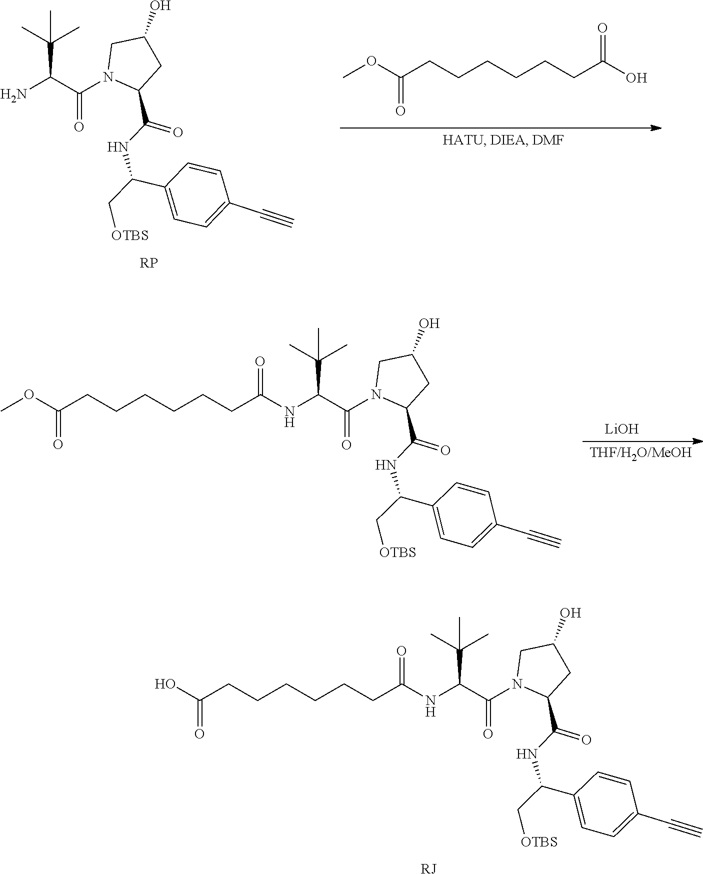

### Step 1—Methyl 8-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-8-oxooctanoate

To a solution of 8-methoxy-8-oxooctanoic acid (206 mg, 1.10 mmol, CAS #3946-32-5) in DMF (2 mL) was added HATU (542 mg, 1.43 mmol) and stirred for 5 min. Then add DIEA (708 mg, 5.48 mmol) and (2S,4R)-1-((S)-2-amino-3,3-dimethylbutanoyl)-N—((R)-2-((tert-butyldimethylsilyl)oxy)-1-(4-ethynylphenyl)ethyl)-4-hydroxypyrrolidine-2-carboxamide (550 mg, 1.10 mmol, Intermediate RP). The mixture was then stirred at 25° C. for 0.5 hr. On completion, the reaction mixture was concentrated under reduced pressure to give a residue. The residue was purified by reversed-phase HPLC (0.8 g/L ammonium bicarbonate) to give the title compound (700 mg, 94% yield) as a white solid. LC-MS (ESI⁺) m/z 672.6 (M+H)⁺.

### Step 2—8-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-8-oxooctanoic Acid

To a solution of methyl 8-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-8-oxooctanoate (300 mg, 446 μmol) in THF (4 mL), H₂O (1 mL) and MeOH (1 mL) was added LiOH·H₂O (93.7 mg, 2.23 mmol). The mixture was then stirred at 25° C. for 2 hrs. On completion, the reaction mixture was added HCl (1N) until the pH was 6, then diluted with water (10 mL) and extracted with dichloromethane (5×2 mL). The combined organic layers were dried over anhydrous sodium sulfate, filtered and the filtrate was concentrated in vacuo to give the title compound (290 mg) as white solid. LC-MS (ESI⁺) m/z 658.5 (M+H)⁺.

### Synthesis of 2-((1H-pyrrolo[2,3-b]pyridin-5-yl)oxy)-N-((4-((3-(4-aminopiperidin-1-yl)propyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide (Intermediate RK)

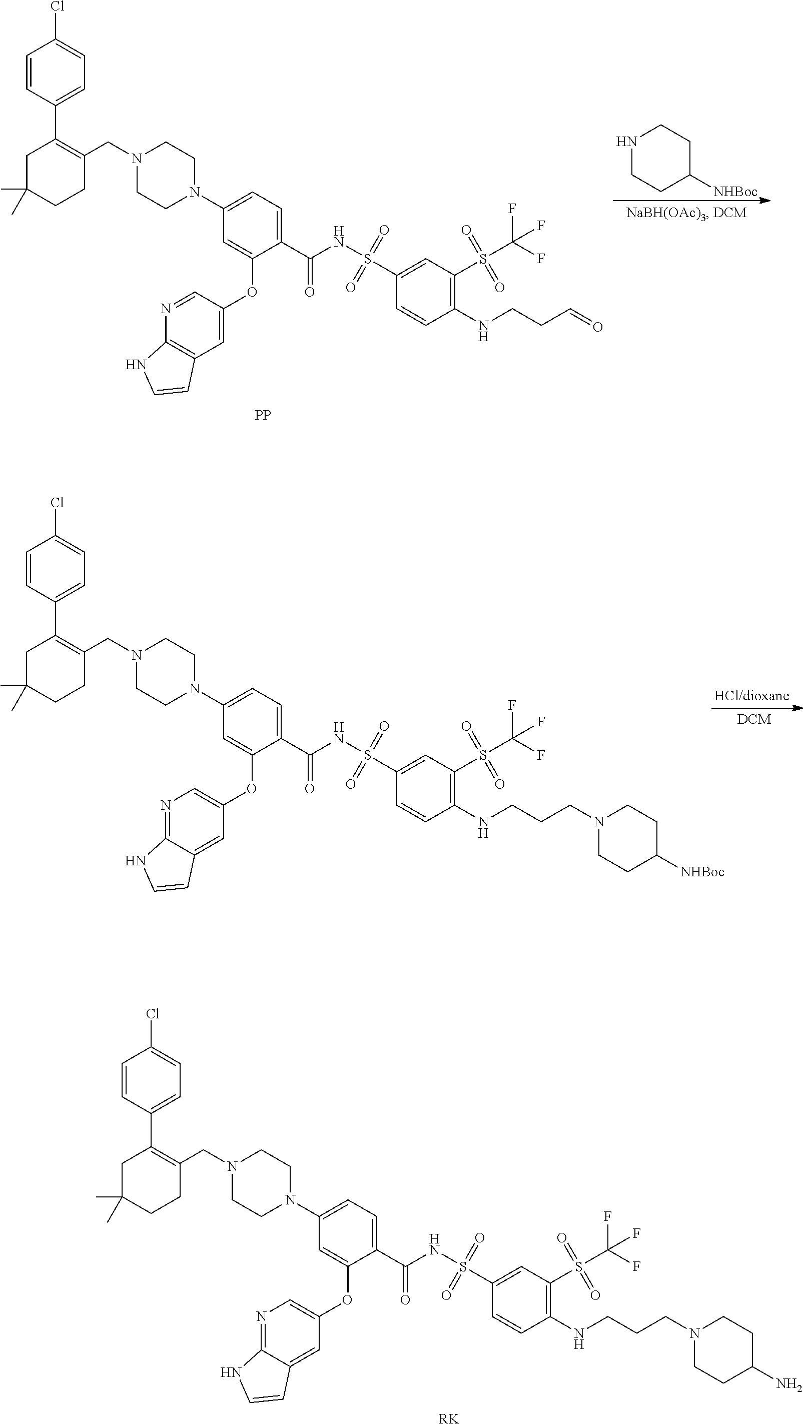 [SMILES: CC1(C)CCC(CN2CCN(c3ccc(C(=O)NS(=O)(=O)c4ccc(NCCC=O)c(S(=O)(=O)C(F)(F)F)c4)c(Oc4cnc5[nH]ccc5c4)c3)CC2)=C(c2ccc(Cl)cc2)C1 ; CC(C)(C)OC(=O)NC1CCNCC1 ; "PP" ; CC1(C)CCC(CN2CCN(c3ccc(C(=O)NS(=O)(=O)c4ccc(NCCCN5CCC(NC(=O)OC(C)(C)C)CC5)c(S(=O)(=O)C(F)(F)F)c4)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(NCCCN5CCC(N)CC5)c(S(=O)(=O)C(F)(F)F)c4)c(Oc4cnc5[nH]ccc5c4)c3)CC2)=C(c2ccc(Cl)cc2)C1 ; "RK"]

### Step 1—Tert-butyl (1-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperidin-4-yl)carbamate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-oxopropyl)amino)-3-((trifluoromethyl)sulfonyl)phenyl)sulfonyl)benzamide (200 mg, 219 μmol, Intermediate PP) in DCM (1 mL) was added tert-butyl piperidin-4-ylcarbamate (43.8 mg, 219 μmol, CAS #73874-95-0) and NaBH(OAc) 3 (60.3 mg, 285 μmol). The mixture was then stirred at 25° C. for 3 hrs. On completion, the reaction mixture was diluted with water (5 mL) and extracted with dichloromethane (5×3 mL). The combined organic layers were washed with brine (15 mL) and dried over anhydrous sodium sulfate, filtered and the filtrate was concentrated in vacuo to give the crude residue. Then the crude residue was purified by column chromatography (SiO₂, Dichloromethane:Methanol=10/1 to 8/1) to give the title compound (180 mg, 69% yield) as a yellow oil. LC-MS (ESI+) m/z 1097.4 (M+H-55)⁺.

### Step 2—2-((1H-pyrrolo[2,3-b]pyridin-5-yl)oxy)-N-((4-((3-(4-aminopiperidin-1-yl)propyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide

To a solution of tert-butyl (1-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperidin-4-yl)carbamate (180 mg, 164 μmol) in DCM (2 mL) was added HCl/dioxane (4 M, 0.5 mL). The mixture was then stirred at 25° C. for 1 hr. On completion, the reaction mixture was concentrated in vacuo to give the title compound (160 mg, HCl) as a yellow solid. LC-MS (ESI⁺) m/z 997.2 (M+H)⁺.

### Synthesis of (R)-2-((1H-pyrrolo[2,3-b]pyridin-5-yl)oxy)-4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-((3-(dimethylamino)propyl)amino)-3-((trifluoromethyl)sulfonyl)phenyl)sulfonyl)benzamide (Intermediate RL)

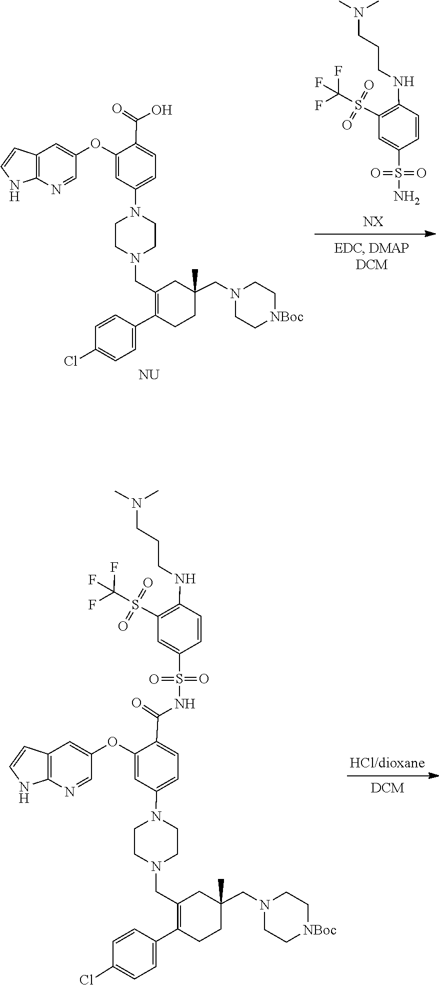

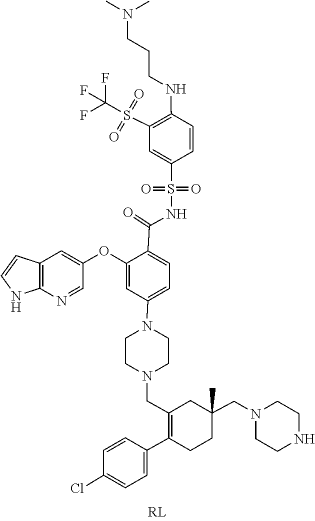 [SMILES: CN(C)CCCNc1ccc(S(=O)(=O)NC(=O)c2ccc(N3CCN(CC4=C(c5ccc(Cl)cc5)CC[C@@](C)(CN5CCNCC5)C4)CC3)cc2Oc2cnc3[nH]ccc3c2)cc1S(=O)(=O)C(F)(F)F]

### Step 1—Tert-butyl (R)-4-((6-((4-(3-((1H-pyrrolo[2,3-b]pyridin-5-yl)oxy)-4-(((4-((3-(dimethylamino)propyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate

To a solution of 4-[4-[[(5R)-5-[(4-tert-butoxycarbonylpiperazin-1-yl)methyl]-2-(4-chlorophenyl)-5-methyl-cyclohexen-1-yl]methyl]piperazin-1-yl]-2-(1H-pyrrolo[2,3-b]pyridin-5-yloxy)benzoic acid (150 mg, 198 μmol, Intermediate NU) in DCM (8 mL) was added EDC (77.1 mg, 496 μmol) and DMAP (72.8 mg, 596 μmol). Then 4-[3-(dimethylamino)propylamino]-3-(trifluoromethylsulfonyl)benzenesulfonamide (100 mg, 258 μmol, Intermediate NX) was added and the mixture was stirred at 25° C. for 2 hrs. On completion, the reaction mixture was quenched with water (8 mL) and extracted by DCM (30 mL). The combined organic layers were washed with brine (10 mL) and dried over anhydrous sodium sulfate, filtered and the filtrate was concentrated in vacuo to give the a residue. The residue was purified by reversed-phase HPLC (0.1% FA condition) to give the title compound (80 mg, 32% yield, FA) as a yellow solid. LC-MS (ESI⁺) m/z 1126.6 (M+H)⁺.

### Step 2—(R)-2-((1H-pyrrolo[2,3-b]pyridin-5-yl)oxy)-4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-((3-(dimethylamino)propyl)amino)-3-((trifluoromethyl)sulfonyl)phenyl)sulfonyl)benzamide

To a solution of tert-butyl 4-[[(1R)-4-(4-chlorophenyl)-3-[[4-[4-[[4-[3-(dimethylamino)propylamino]-3-(trifluoromethylsulfonyl)phenyl]sulfonylcarbamoyl]-3-(1H-pyrrolo[2,3-b]pyridin-5-yloxy)phenyl]piperazin-1-yl]methyl]-1-methyl-cyclohex-3-en-1-yl]methyl]piperazine-1-carboxylate (70 mg, 62 μmol) in DCM (4 mL) was added HCl/dioxane (4 M, 1 mL). The mixture was stirred at 25° C. for 2 hrs. On completion, the reaction mixture was concentrated in vacuo to remove solvent to give the title compound as a light yellow solid (60 mg) LC-MS (ESI⁺) m/z 1026.3 (M+H)⁺.

### Synthesis of 9-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)nonanoic Acid (Intermediate RM)

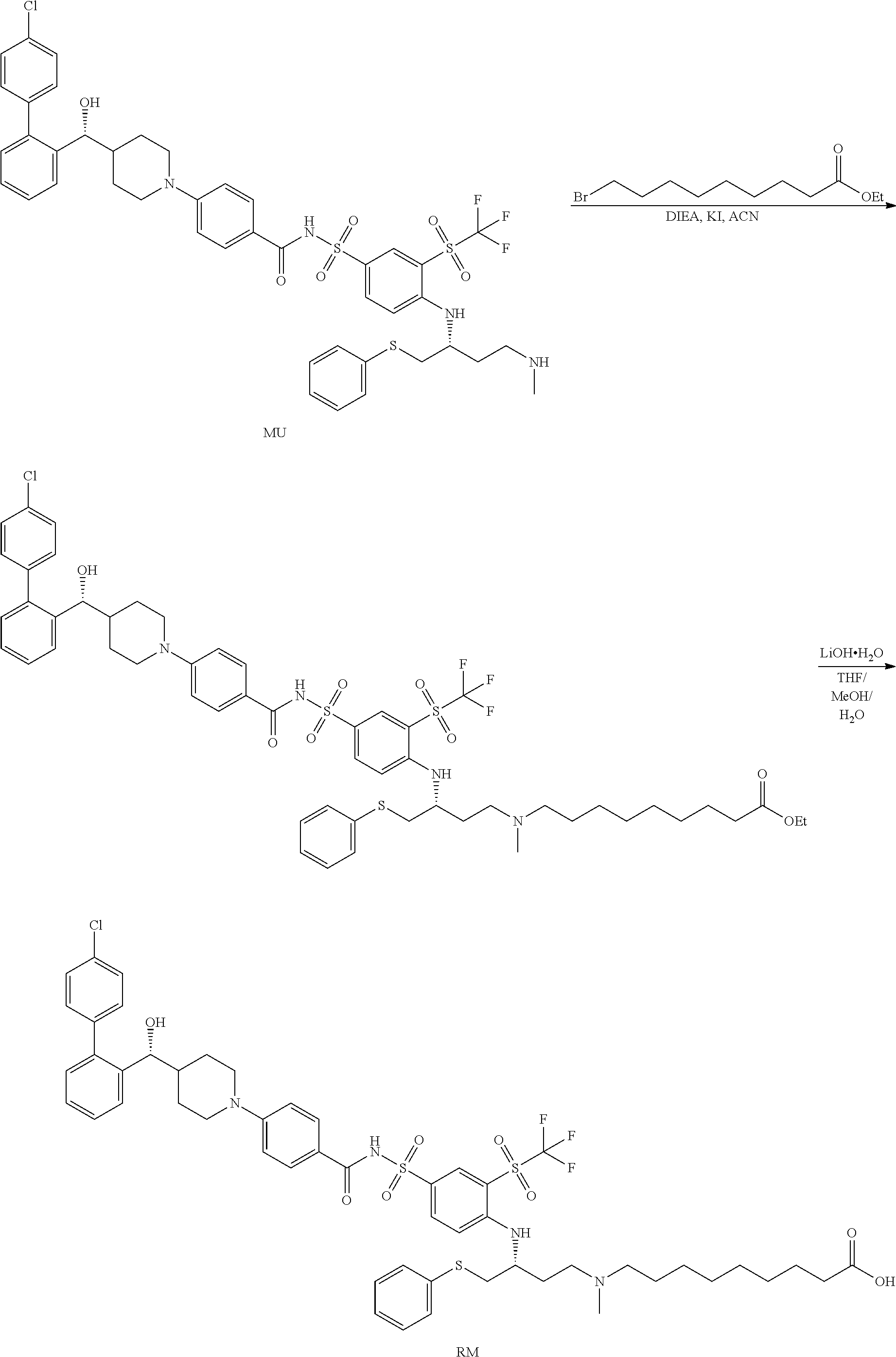

### Step 1—Ethyl 9-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino) nonanoate

To a solution of 4-(4-((R)-(4′-chloro-[1,1′-biphenyl]-2-yl)(hydroxy)methyl)piperidin-1-yl)-N-((4-(((R)-4-(methylamino)-1-(phenylthio)butan-2-yl)amino)-3-((trifluoromethyl)sulfonyl)phenyl)sulfonyl)benzamide (220 mg, 235 μmol, HCl, Intermediate MU) in ACN (5 mL) was added KI (3.89 mg, 23.5 μmol), DIEA (152 mg, 1.17 mmol) and ethyl 9-bromononanoate (124 mg, 469 μmol, CAS #28598-81-4). The mixture was then stirred at 50° C. for 24 hrs. On completion, the reaction mixture was filtered to give a filtrate. The filtrate was purified by reversed-phase HPLC (0.1% HCl condition) to give the title compound (120 mg, 41% yield) as a white solid. LC-MS (ESI⁺) m/z 1085.4 (M+H)⁺.

### Step 2—9-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)nonanoic Acid

To a solution of ethyl 9-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino) nonanoate (100 mg, 92.1 μmol) in MeOH (1 mL), THF (4 mL) and H₂O (1 mL) was added LiOH H₂O (11.6 mg, 276 μmol). The mixture was then stirred at 25° C. for 2 hrs. On completion, the reaction mixture was quenched with 1M HCl until the pH was 6, and then extracted with ethyl acetate (10 mL). The combined organic lavers were dried over anhydrous sodium sulfate, filtered and the filtrate was concentrated under reduced pressure to give the title compound (80 mg) as a white solid. LC-MS (ESI⁺) m/z 1057.3 (M+H)⁺.

### Synthesis of 8-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)octanoic Acid (Intermediate RN)

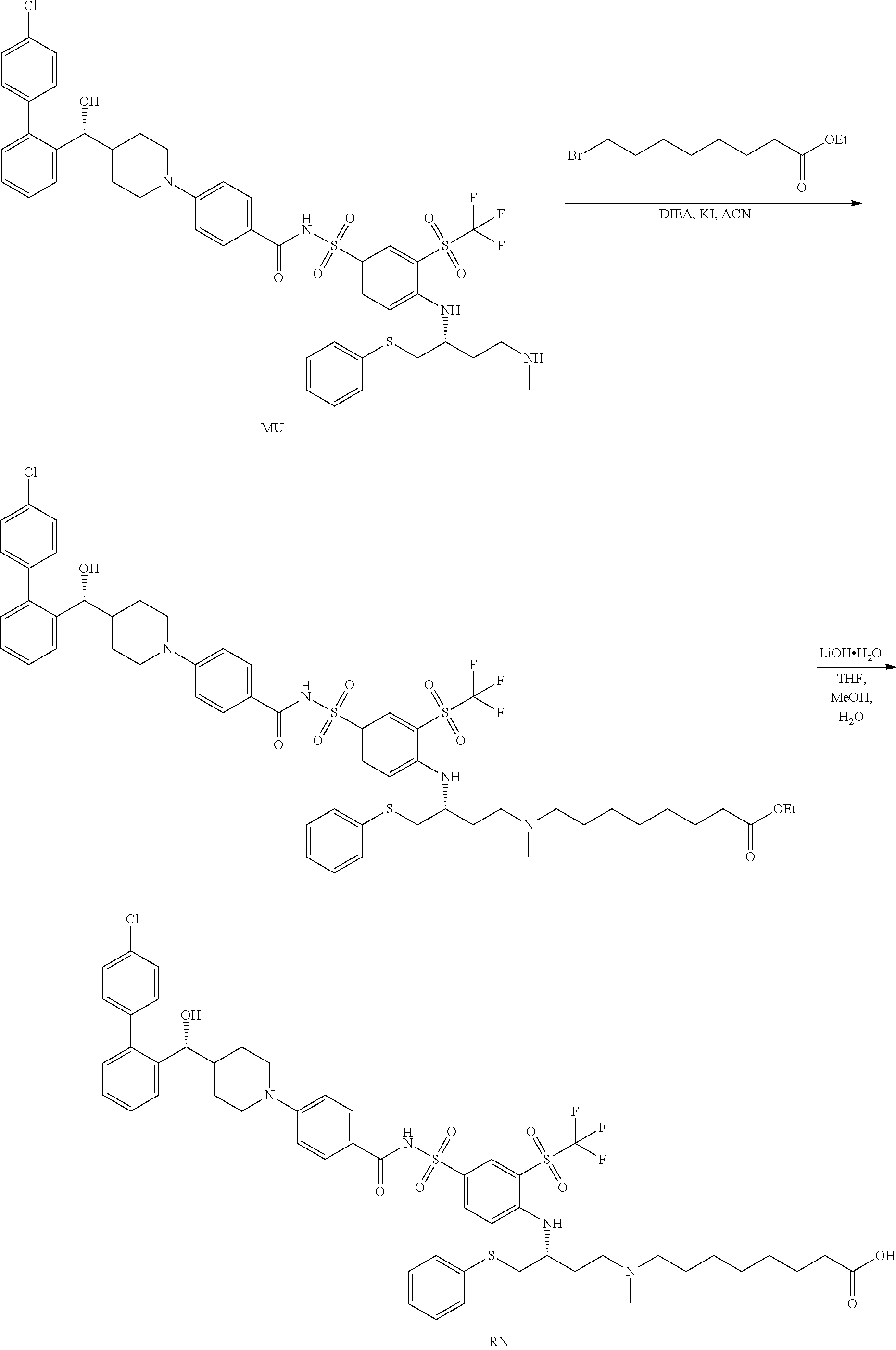

### Step 1—Ethyl 8-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)octanoate

To a solution of 4-(4-((R)-(4′-chloro-[1,1′-biphenyl]-2-yl)(hydroxy)methyl)piperidin-1-yl)-N-((4-(((R)-4-(methylamino)-1-(phenylthio)butan-2-yl)amino)-3-((trifluoromethyl)sulfonyl)phenyl)sulfonyl)benzamide (220 mg, 234 μmol, HCl, Intermediate MU) in ACN (2 mL) was added DIEA (151 mg, 1.17 mmol, 204 uL), KI (3.89 mg, 23.5 μmol) and ethyl 8-bromooctanoatc (118 mg, 469 μmol, CAS #29823-21-0). The mixture was then stirred at 50° C. for 24 hrs. On completion, the reaction mixture was concentrated in vacuo to give the crude residue. The crude residue was purified by reversed-phase HPLC (0.1% HCl condition) to give the title compound (120 mg, 56% yield) as a white solid. LC-MS (ESI+) m/z 536.4 (M/2+H)⁺.

### Step 2—8-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)octanoic Acid

To a solution of ethyl 8-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)octanoate (130 mg, 121 μmol) in THF (2 mL), MeOH (2 mL) and H₂O (0.8 mL) was added LiOH·H₂O (15.3 mg, 364 μmol). The mixture was then stirred at 40° C. for 1 hr. On completion, the reaction mixture was concentrated in vacuo to remove solvent. The reaction mixture was diluted with water (5 mL) and extracted with dichloromethane (3×5 mL). The combined organic layers were washed with brine (15 mL) and dried over anhydrous sodium sulfate, filtered and the filtrate was concentrated in vacuo to give the title compound (120 mg) as a white solid. LC-MS (ESI+) m/z 1043.3 (M+H)⁺.

### Synthesis of 6-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)hexanoic Acid (Intermediate RO)

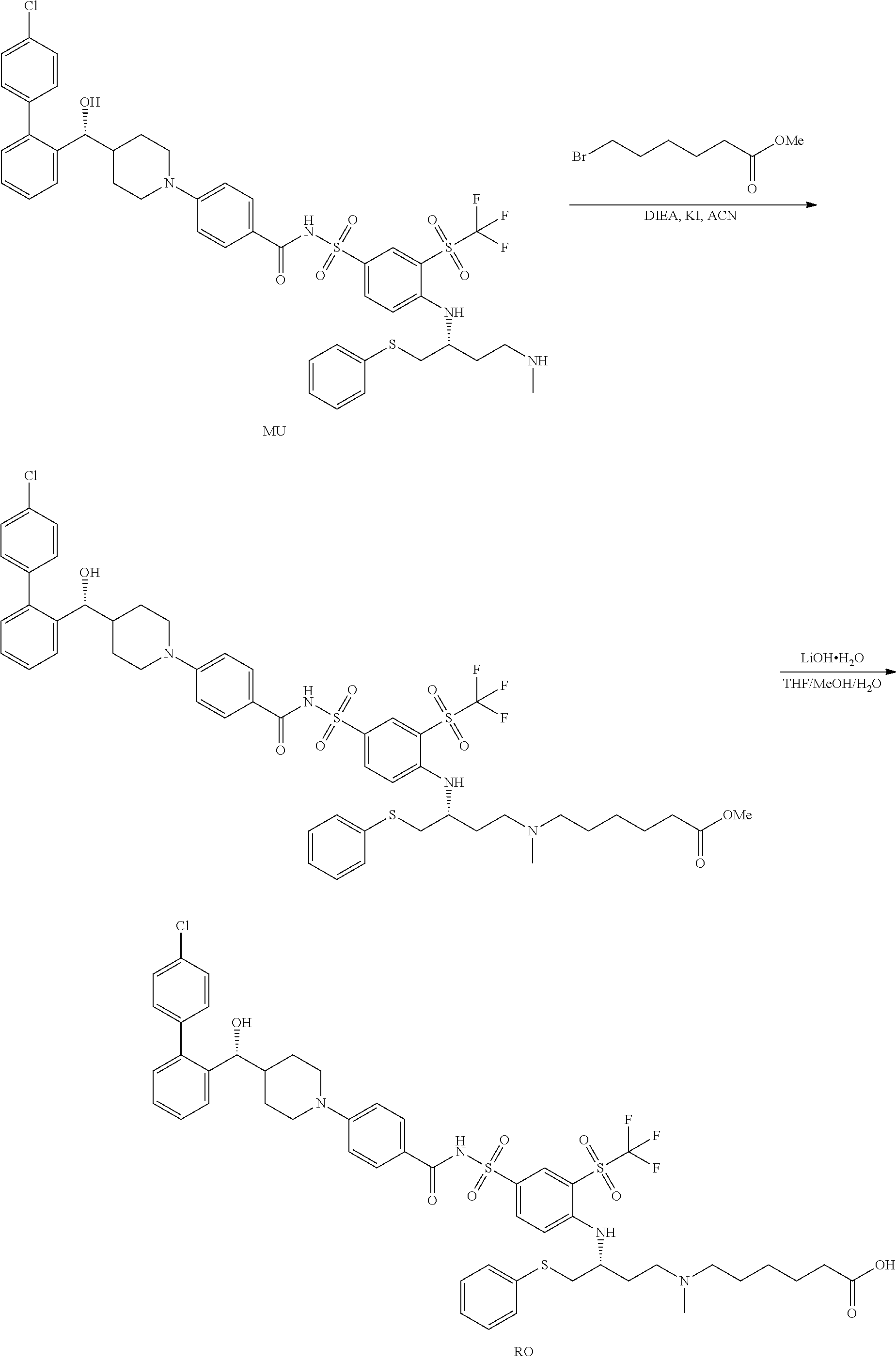

### Step 1—Methyl 6-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)hexanoate

To a solution of 4-[4-[(R)-[2-(4-chlorophenyl)phenyl]-hydroxy-methyl]-1-piperidyl]-N-[4-[[(1R)-3-(methylamino)-1-(phenylsulfanylmethyl)propyl]amino]-3-(trifluoromethylsulfonyl)phenyl]sulfonyl-benzamide (220 mg, 234.56 μmol, HCl, Intermediate MU) in ACN (5 mL) was added DIEA (151 mg, 1.17 mmol), KI (3.89 mg, 23.4 μmol) and methyl 6-bromohexanoate (98.1 mg, 469.12 μmol). The mixture was stirred at 50° C. for 12 hrs. On completion, the reaction mixture was concentrated in vacuo and the crude residue was purified by reversed-phase HPLC (0.1% HCl condition) to give the title compound (150 mg, 59% yield, HCl) as a white solid. LC-MS (ESI+) m/z 515.4 (M/2+H)⁺.

### Step 2—6-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino) hexanoic Acid

To a solution of methyl 6-(((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)(methyl)amino)hexanoate (150 mg, 145 μmol) in THF (2 mL), MeOH (0.5 mL) and H₂O (0.5 mL) was added LiOH·H₂O (18.3 mg, 437 μmol). The mixture was then stirred at 40° C. for 12 hrs. On completion, the reaction mixture was concentrated under reduced pressure to give a residue. The residue was purified by prep-HPLC (neutral condition) to give the title compound (30 mg, 19% yield) as a white solid. LC-MS (ESI+) m/z 1015.2 (M+H)⁺.

### Synthesis of (2S,4R)-1-((S)-2-amino-3,3-dimethylbutanoyl)-N—((R)-2-((tert-butyldimethylsilyl)oxy)-1-(4-ethynylphenyl)ethyl)-4-hydroxypyrrolidine-2-carboxamide (Intermediate RP)

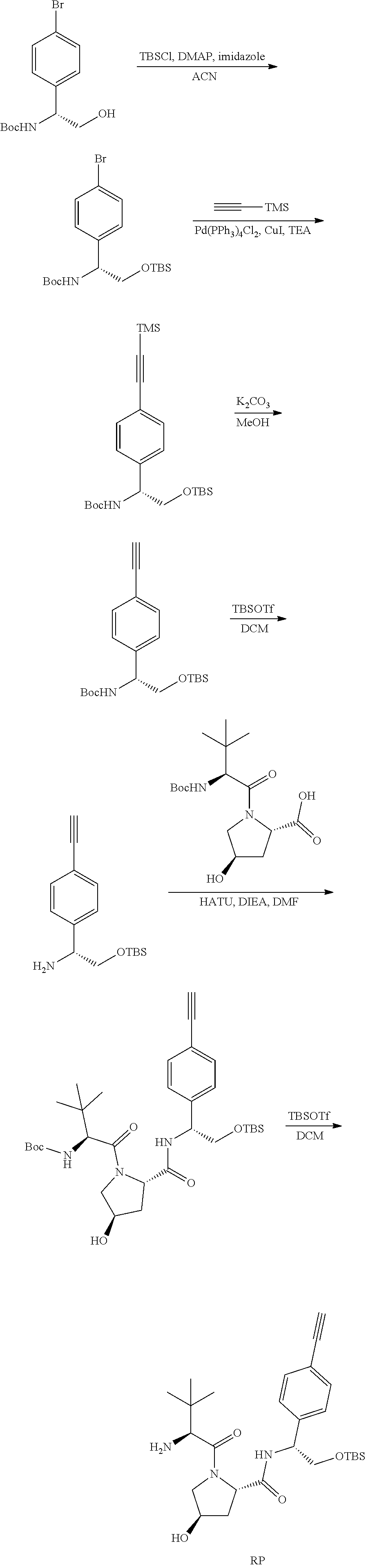

### Step 1—Tert-butyl (R)-(1-(4-bromophenyl)-2-((tert-butyldimethylsilyl)oxy)ethyl)carbamate

To a solution of tert-butyl (R)-(1-(4-bromophenyl)-2-hydroxyethyl)carbamate (85.0 g, 268 mmol, CAS #849178-85-4) in ACN (850 mL) was added TBSCl (48.6 g, 322 mmol, 39.5 mL), DMAP (39.4 g, 322 mmol) and imidazole (27.4 g, 403 mmol). The mixture was then stirred at 25° C. for 1.5 hrs. On completion, the mixture was diluted with EtOAc (500 mL). Then the organic layer was washed with saturated NaHCO₃ solution (300 mL×2), brine (200 mL), dried over Na₂SO₄, filtered and concentrated under vacuum to give the title compound (125.5 g) as colorless oil. ¹H NMR (CDCl₃, 400 MHz) δ 7.44 (d, J=8.0 Hz, 2H), 7.17 (d, J=8.4 Hz, 2H), 5.28 (s, 2H), 4.64 (s, 1H), 3.84-3.88 (m, 1H), 3.68 (d, J=5.6 Hz, 1H), 1.42 (s, 9H), 0.84 (s, 9H), −0.04 (s, 3H), −0.07 (s, 3H).

### Step 2—Tert-butyl (R)-(2-((tert-butyldimethylsilyl)oxy)-1-(4-((trimethylsilyl)ethynyl)phenyl)ethyl)carbamate

To a solution of tert-butyl (R)-(1-(4-bromophenyl)-2-((tert-butyldimethylsilyl)oxy)ethyl)carbamate (125 g, 291 mmol) and ethynyl(trimethyl) silane (85.9 g, 874 mmol, 121 mL) in TEA (908 g, 8.98 mol, 1.25 L) was added CuI (2.78 g, 14.5 mmol) and Pd(PPh₃)₂Cl₂ (6.14 g. 8.75 mmol). The mixture was then stirred at 80° C. for 12 hrs. On completion, the mixture was diluted with EtOAc (500 mL) and water (300 mL), filtered, and separated. The organic layer was washed with brine (200 mL), dried over Na₂SO₄, filtered and concentrated under vacuum. The crude was purified by column chromatography (Petroleum ether/Ethyl acetate=0:1-10:1, R_{f}=0.5) to give the title compound (110 g, 84% yield) as a light yellow solid. LC-MS (ESI+) m/z 392.1 (M-56+H)⁺; ¹H NMR (CDCl₃, 400 MHz) δ 7.42 (d, J=6.8 Hz, 2H), 7.22 (d, J=8.4 Hz, 2H), 5.28 (s, 2H), 4.69 (s, 1H), 3.85-3.88 (m, 1H), 3.70 (s, 1H), 1.42 (s, 9H), 0.83 (s, 9H), 0.25 (s, 9H), −0.06 (s, 3H), −0.09 (s, 3H).

### Step 3—Tert-butyl (R)-(2-((tert-butyldimethylsilyl)oxy)-1-(4-ethynylphenyl)ethyl)carbamate

To a solution of tert-butyl (R)-(2-((tert-butyldimethylsilyl)oxy)-1-(4-((trimethylsilyl)ethynyl)phenyl)ethyl)carbamate (105 g, 234 mmol) in MeOH (700 mL) was added K₂CO₃ (64.8 g, 469 mmol). The mixture was then stirred at 25° C. for 2 hrs. On completion, the mixture was diluted with water (1000 mL), and extracted with EtOAc (300 mL×2). The combined organic layer was washed with brine (200 mL), dried over Na₂SO₄, filtered and concentrated under vacuum to give the title compound (85.5 g, 97% yield) as yellow oil. LC-MS (ESI+) m/z 320.2 (M-56+H)⁺; ¹H NMR (CDCl₃, 400 MHz) δ 7.45 (d, J=8.4 Hz, 2H), 7.25 (d, J=8.0 Hz, 2H), 5.30 (s, 1H), 4.69 (s, 1H), 3.85-3.89 (m, 1H), 3.71 (s, 1H), 3.05 (s, 1H), 1.43 (s, 9H), 0.83 (s, 9H), −0.06 (s, 3H), −0.09 (s, 3H).

### Step 4—(R)-2-((tert-butyldimethylsilyl)oxy)-1-(4-ethynylphenyl)ethan-1-amine

To a solution of Tert-butyl (R)-(2-((tert-butyldimethylsilyl)oxy)-1-(4-ethynylphenyl)ethyl)carbamate (70.0 g, 186 mmol) in DCM (700 mL) was added TBSOTf (59.1 g, 223 mmol, 51.4 mL), and the mixture was stirred at 25° C. for 30 min. On completion, the mixture was diluted with water (100 mL), adjusted to pH-7 with saturated NaHCO₃, and separated. The organic layer was washed with brine (100 mL), dried over Na₂SO₄, filtered and concentrated under vacuum. The crude was purified by column chromatography (DCM/MeOH=0:1-50:1, R_{f}=0.4) to give the title compound (51.0 g. 99% yield) as yellow oil. LC-MS (ESI+) m/z 276.1 (M+H)⁺. ¹H NMR (CDCl₃, 400 MHz) δ 7.45 (d, J=8.4 Hz, 2H), 7.33 (d, J=8.4 Hz, 2H), 4.07 (dd, J=4.0 Hz, 8.0 Hz, 1H), 3.70 (dd, J=4.0 Hz, 9.6 Hz, 1H), 3.50 (dd, J=8.4 Hz, 10.0 Hz, 1H), 3.05 (s, 1H), 0.89 (s, 9H), 0.02 (s, 6H).

### Step 5−Tert-butyl ((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)carbamate

To a solution of (2S,4R)-1-((S)-2-((tert-butoxycarbonyl)amino)-3,3-dimethylbutanoyl)-4-hydroxypyrrolidine-2-carboxylic acid (57.5 g, 166 mmol, CAS #630421-46-4) in DMF (450 mL) was added HATU (76.1 g, 200 mmol) and DIEA (64.7 g, 500 mmol, 87.2 mL) at 0-5° C. The mixture was stirred at 0-5° C. for 30 min. and then (R)-2-((tert-butyldimethylsilyl)oxy)-1-(4-ethynylphenyl)ethan-1-amine (46.0 g, 166 mmol) was added at 0-5° C. The mixture was then stirred at 25° C. for 1 hr. On completion, the mixture was diluted with water (1500 mL), and extracted with DCM (500 mL×2). The combined organic layer was washed with brine (500 mL), dried over Na₂SO₄, filtered and concentrated under vacuum. The crude product was purified by reverse phase HPLC (0.1% NH₃·H₂O) to give the title compound (66.0 g, 66% yield) as a white solid. LC-MS (ESI+) m/z 602.5 (M+H)⁺.

### Step 6—(2S,4R)-1-((S)-2-amino-3,3-dimethylbutanoyl)-N—((R)-2-((tert-butyldimethylsilyl)oxy)-1-(4-ethynylphenyl)ethyl)-4-hydroxypyrrolidine-2-carboxamide

To a solution of tert-butyl ((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)carbamate (70.0 g, 116 mmol) in DCM (600 mL) was added TBSOTf (61.4 g, 232 mmol, 53.4 mL), and the mixture was stirred at 25° C. for 0.5 hr. On completion, the mixture was diluted with water (100 mL), adjusted to pH=7 with saturated NaHCO₃, and separated. The organic layer was washed with brine (300 mL), dried over Na₂SO₄, filtered and concentrated under vacuum. The crude was purified by pre-HPLC (column: Kromasil Eternity XT 250*80 mm*10 μm; mobile phase: [water (ammonia hydroxide v/v)-ACN]; B %: 45%-75%, 20 min) to give the title compound (20.5 g, 35% yield) as an off-white solid. LC-MS (ESI+) m/z 502.5 (M+H)⁺. ¹H NMR (CDCl₃, 400 MHz) δ 7.47 (d, J=8.0 Hz, 2H), 7.31 (d, J=8.0 Hz, 2H), 7.17 (d, J=8.0 Hz, 1H), 5.06-5.11 (m, 1H), 4.56 (t, J=7.2 Hz, 2H), 3.88-3.89 (m, 1H), 3.70-3.78 (m, 2H), 3.54 (d, J=2.0 Hz, 1H), 3.29 (s, 1H), 3.07 (s, 1H), 2.34-2.39 (m, 1H), 1.96-2.01 (m, 1H), 1.00 (s, 9H), 0.87 (s, 9H), 0.08 (s, 6H).

### Synthesis of 7-((3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)(methyl)amino)heptanoic Acid (Intermediate RQ)

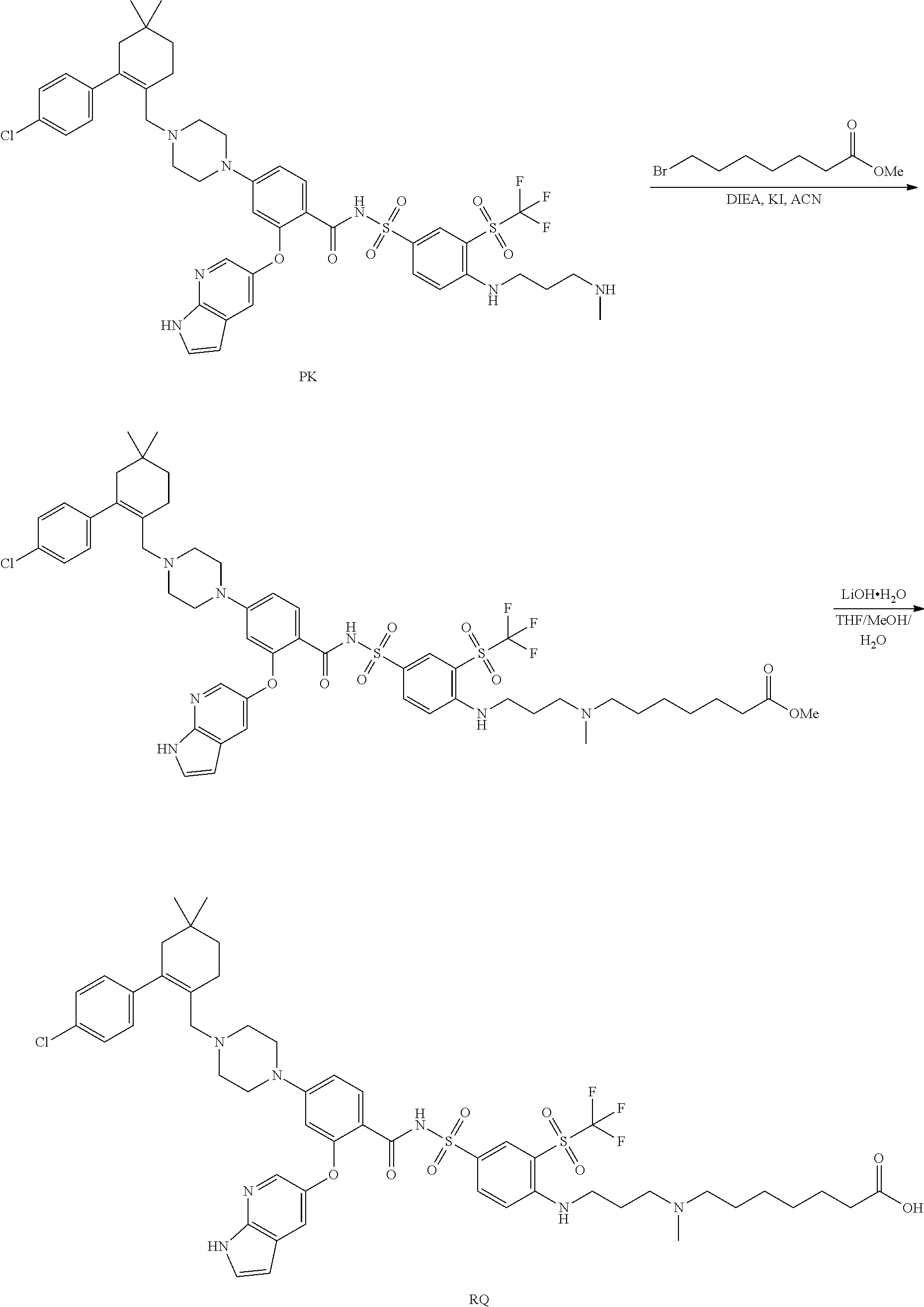

### Step 1—Methyl 7-((3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)(methyl)amino)heptanoate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-(methylamino)propyl)amino)-3-((trifluoromethyl)sulfonyl)phenyl)sulfonyl)benzamide (300 mg, 323 μmol, Intermediate PK) in ACN (6 mL) was added KI (5.36 mg, 32.3 μmol), DIEA (209 mg, 1.62 mmol) and methyl 7-bromoheptanoate (144 mg, 646 μmol, CAS #54049-24-0). The mixture was then stirred at 50° C. for 24 hrs. On completion, the reaction mixture was filtered to give a filtrate. The filtrate was purified by reversed-phase HPLC (0.1% HCl condition) to the title compound (210 mg, 57% yield) as a white solid. LC-MS (ESI⁺) m/z 1070.3 (M+H)⁺.

### Step 2—7-((3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)(methyl)amino)heptanoic Acid

To a solution of methyl 7-((3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)(methyl)amino)heptanoate (210 mg, 196 μmol) in MeOH (1 mL), THF (4 mL) and H₂O (1 mL) was added LiOH·H₂O (24.7 mg, 588 μmol). The mixture was then stirred at 25° C. for 2 hrs. On completion, the reaction mixture was quenched by addition of 1M HCl until the pH was 6, and then extracted with ethyl acetate (10 mL). The combined organic layers were dried over anhydrous sodium sulfate, filtered and the filtrate was concentrated under reduced pressure to give the title compound (200 mg) as a white solid. LC-MS (ESI⁺) m/z 1056.4 (M+H)⁺.

### Synthesis of (2S,4R)—N—((R)-2-((tert-butyldimethylsilyl)oxy)-1-(4-ethynylphenyl)ethyl)-1-((S)-3,3-dimethyl-2-(8-oxooctanamido)butanoyl)-4-hydroxypyrrolidine-2-carboxamide (Intermediate RR)

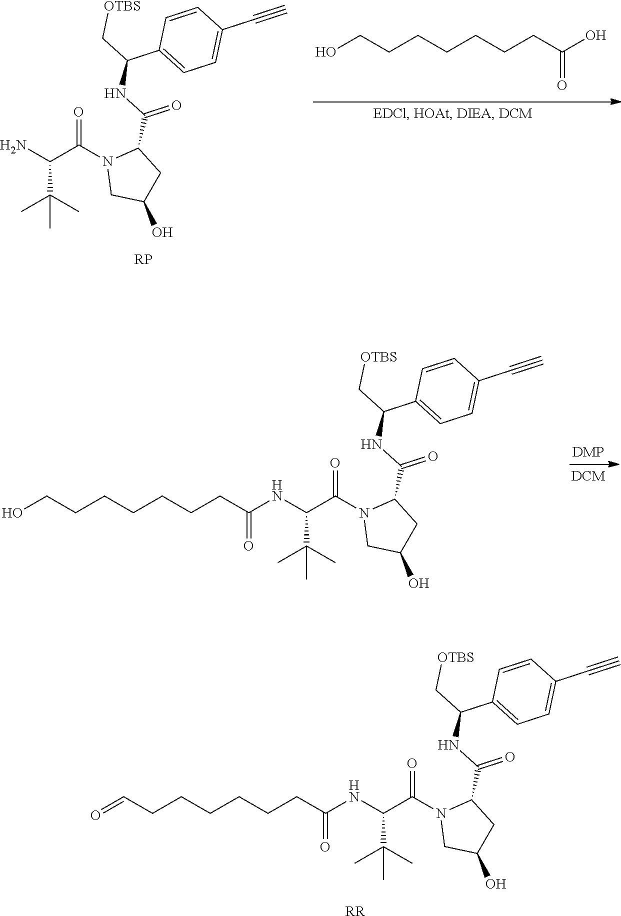 [SMILES: C#Cc1ccc([C@H](CO[Si](C)(C)C(C)(C)C)NC(=O)[C@@H]2C[C@@H](O)CN2C(=O)[C@@H](N)C(C)(C)C)cc1 ; O=C(O)CCCCCCCO ; C#Cc1ccc([C@H](CO[Si](C)(C)C(C)(C)C)NC(=O)[C@@H]2C[C@@H](O)CN2C(=O)[C@@H](NC(=O)CCCCCCCO)C(C)(C)C)cc1 ; C#Cc1ccc([C@H](CO[Si](C)(C)C(C)(C)C)NC(=O)[C@@H]2C[C@@H](O)CN2C(=O)[C@@H](NC(=O)CCCCCCC=O)C(C)(C)C)cc1 ; "RR"]

### Step 1—(2S,4R)—N—((R)-2-((tert-butyldimethylsilyl)oxy)-1-(4-ethynylphenyl)ethyl)-4-hydroxy-1-((S)-2-(8-hydroxyoctanamido)-3,3-dimethylbutanoyl)pyrrolidine-2-carboxamide

To a solution of 8-hydroxyoctanoic acid (192 mg, 1.20 mmol, CAS #764-89-6) in DCM (10 mL) was added DIEA (1.67 g, 13.0 mmol, 2.26 mL), EDCI (478 mg, 2.49 mmol). HOAt (339 mg, 2.49 mmol, 349 uL) and (2S,4R)-1-((S)-2-amino-3,3-dimethylbutanoyl)-N—((R)-2-((tert-butyldimethylsilyl)oxy)-1-(4-ethynylphenyl)ethyl)-4-hydroxypyrrolidine-2-carboxamide (500 mg, 997 μmol, Intermediate RP). The reaction was then stirred at 25° C. for 2 hrs. On completion, the reaction mixture was concentrated under reduced pressure to give a residue. The residue was purified by prep-HPLC (0.1% FA condition) to give the title compound (500 mg, 77% yield) as a white oil. LC-MS (ESI⁺) m/z 644.6. (M+H)⁺.

### Step 2—(2S,4R)—N—((R)-2-((tert-butyldimethylsilyl)oxy)-1-(4-ethynylphenyl)ethyl)-1-((S)-3,3-dimethyl-2-(8-oxooctanamido)butanoyl)-4-hydroxypyrrolidine-2-carboxamide

To a solution of (2S,4R)—N—((R)-2-((tert-butyldimethylsilyl)oxy)-1-(4-ethynylphenyl)ethyl)-4-hydroxy-1-((S)-2-(8-hydroxyoctanamido)-3,3-dimethylbutanoyl)pyrrolidine-2-carboxamide (300 mg, 466 μmol) in anhydrous DCM (3 mL) was added DMP (296 mg, 699 μmol, 216 uL) at 0° C., then the reaction was stirred at 0° C. for 30 mins. On completion, the reaction mixture was filtered and the filtrate was concentrated in vacuo to give the crude residue. The crude residue was purified by reversed-phase HPLC (0.8 g/L ammonium bicarbonate) to give the title compound (100 mg, 33% yield) as a white solid. LC-MS (ESI⁺) m/z 642.3. (M+H)⁺.

### Synthesis of (2S,4R)-1-((S)-2-amino-3,3-dimethylbutanoyl)-N—((R)-1-(4-cyanophenyl)-2-hydroxyethyl)-4-hydroxypyrrolidine-2-carboxamide (Intermediate RS)

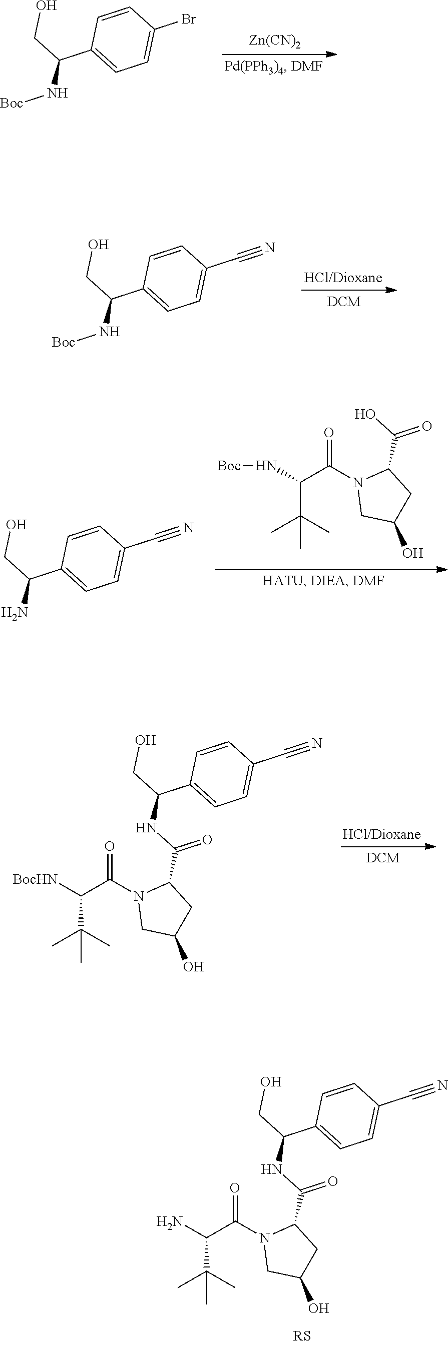

### Step 1—Tert-butyl (R)-(1-(4-cyanophenyl)-2-hydroxyethyl)carbamate

A mixture of tert-butyl (R)-(1-(4-bromophenyl)-2-hydroxyethyl)carbamate (2 g, 6.33 mmol, CAS #849178-85-4), dicyanozinc (1.49 g, 12.7 mmol, CAS #557-21-1), Pd(PPh₃)₄ (1.46 g, 1.27 mmol) in DMF (20 mL) was degassed and purged with N₂ three times, and then the mixture was stirred at 120° C. for 12 hrs under N₂ atmosphere. On completion, the mixture was concentrated in vacuo to give the crude residue. The residue was purified by column chromatography (SiO₂, Petroleum ether/Ethyl acetate=10/1 to 1:1) to give the title compound (1.5 g, 38% yield) as a white solid. LC-MS (ESI+) m/z 285.2 (M+23)⁺.

### Step 2—(R)-4-(1-amino-2-hydroxyethyl)benzonitrile

To a solution of tert-butyl (R)-(1-(4-cyanophenyl)-2-hydroxyethyl)carbamate (1.5 g, 5.72 mmol) in DCM (10 mL) was added HCl/dioxane (4 M, 7.50 mL). The mixture was then stirred at 25° C. for 2 hrs. On completion, the mixture was concentrated to remove organic solvent to give the title compound (120 mg, HCl) as a yellow solid. LC-MS (ESI⁺) m/z 163.2 (M+H)⁺.

### Step 3—Tert-butyl ((S)-1-((2S,4R)-2-(((R)-1-(4-cyanophenyl)-2-hydroxyethyl)carbamoyl)-4-hydroxypyrrolidin-1-yl)-3,3-dimethyl-1-oxobutan-2-yl)carbamate

To a solution of (2S,4R)-1-((S)-2-((tert-butoxycarbonyl)amino)-3,3-dimethylbutanoyl)-4-hydroxypyrrolidine-2-carboxylic acid (1.8 g, 5.23 mmol) in DMF (10 mL) was added DIEA (3.38 g, 26.1 mmol), HATU (1.99 g, 5.23 mmol) and (R)-4-(1-amino-2-hydroxyethyl)benzonitrile (1.04 g, 5.23 mmol, HCl). The mixture was then stirred at 25° C. for 10 min. On completion, the reaction mixture was filtered to give a filtrate. The filtrate was purified by reversed-phase HPLC (0.1% HCl condition) to the title compound (930 mg, 34% yield) as a white solid. LC-MS (ESI⁺) m/z 489.3 (M+H)+.

### Step 4—(2S,4R)-1-((S)-2-amino-3,3-dimethylbutanoyl)-N—((R)-1-(4-cyanophenyl)-2-hydroxyethyl)-4-hydroxypyrrolidine-2-carboxamide

To a solution of tert-butyl ((S)-1-((2S,4R)-2-(((R)-1-(4-cyanophenyl)-2-hydroxyethyl)carbamoyl)-4-hydroxypyrrolidin-1-yl)-3,3-dimethyl-1-oxobutan-2-yl)carbamate (930 mg, 1.90 mmol) in DCM (10 mL) was added HCl/Dioxane (4 M, 4.65 mL). The mixture was then stirred at 25° C. for 2 hrs. On completion, the mixture was concentrated to remove organic solvent to give the title compound (800 mg, HCl) as a white solid. LC-MS (ESI⁺) m/z 389.2 (M+H)⁺.

### Synthesis of 7-(((S)-1-((2S,4R)-2-(((R)-1-(4-cyanophenyl)-2-hydroxyethyl)carbamoyl)-4-hydroxypyrrolidin-1-yl)-3,3-dimethyl-1-oxobutan-2-yl)amino)-7-oxoheptanoic Acid (Intermediate RT)

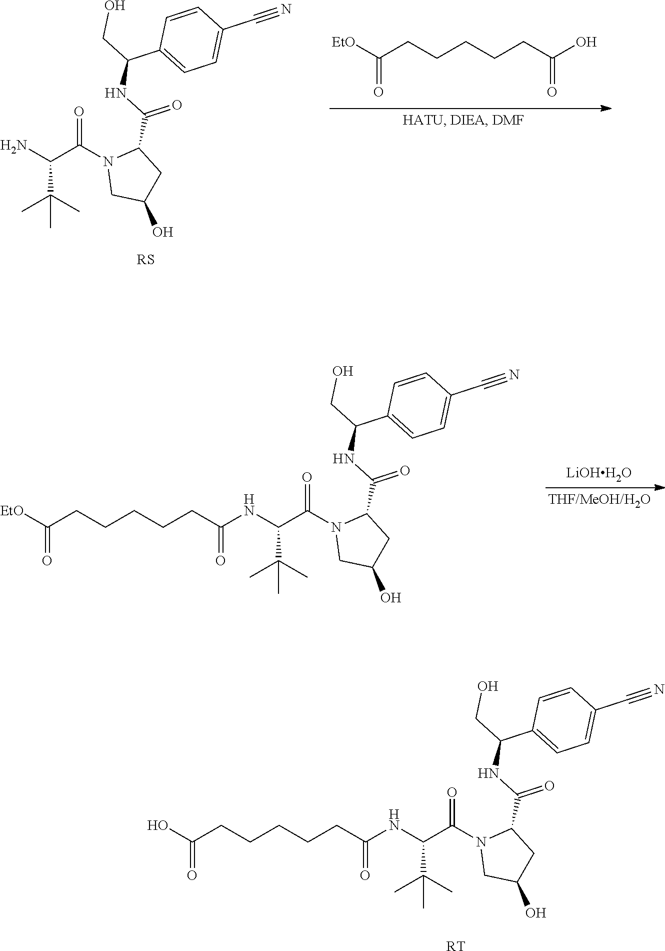

### Step 1—Ethyl 7-(((S)-1-((2S,4R)-2-(((R)-1-(4-cyanophenyl)-2-hydroxyethyl)carbamoyl)-4-hydroxypyrrolidin-1-yl)-3,3-dimethyl-1-oxobutan-2-yl)amino)-7-oxoheptanoate

To a solution of tert-butyl 7-ethoxy-7-oxoheptanoic acid (350 mg, 1.86 mmol, CAS #33018-91-6) in DMF (10 mL) was added DIEA (1.20 g, 9.30 mmol, 1.62 mL), HATU (919 mg, 2.42 mmol) and (2S,4R)-1-((S)-2-amino-3,3-dimethylbutanoyl)-N—((R)-1-(4-cyanophenyl)-2-hydroxyethyl)-4-hydroxypyrrolidine-2-carboxamide (790 mg, 1.86 mmol, HCl, Intermediate RS). The mixture was then stirred at 25° C. for 10 min. On completion, the reaction mixture was filtered to give a filtrate. The filtrate was purified by reversed-phase HPLC (0.8 g/L ammonium bicarbonate) to the title compound (580 mg, 50% yield) as a white solid. LC-MS (ESI⁺) m/z 559.3 (M+H)⁺.

### Step 2—7-(((S)-1-((2S,4R)-2-(((R)-1-(4-cyanophenyl)-2-hydroxyethyl)carbamoyl)-4-hydroxypyrrolidin-1-yl)-3,3-dimethyl-1-oxobutan-2-yl)amino)-7-oxoheptanoic Acid

To a solution of ethyl 7-(((S)-1-((2S,4R)-2-(((R)-1-(4-cyanophenyl)-2-hydroxyethyl)carbamoyl)-4-hydroxypyrrolidin-1-yl)-3,3-dimethyl-1-oxobutan-2-yl)amino)-7-oxoheptanoate (580 mg, 1.04 mmol) in MeOH (1 mL), THF (4 mL) and H₂O (1 mL) was added LiOH·H₂O (131 mg, 3.11 mmol). The mixture was then stirred at 25° C. for 2 hrs. On completion, the reaction mixture was quenched by addition with 1M HCl until the pH was 6, and then diluted with H₂O (10 mL) and extracted with ethyl acetate (10 mL). The combined organic layers were dried over anhydrous sodium sulfate, filtered and the filtrate was concentrated under reduced pressure to give the title compound (500 mg) as a white solid. LC-MS (ESI⁺) m/z 531.2 (M+H)⁺.

### Synthesis of (2S,4R)-1-((S)-2-amino-3,3-dimethylbutanoyl)-N—((R)-1-(4-chlorophenyl)-2-hydroxyethyl)-4-hydroxypyrrolidine-2-carboxamide (Intermediate RU)

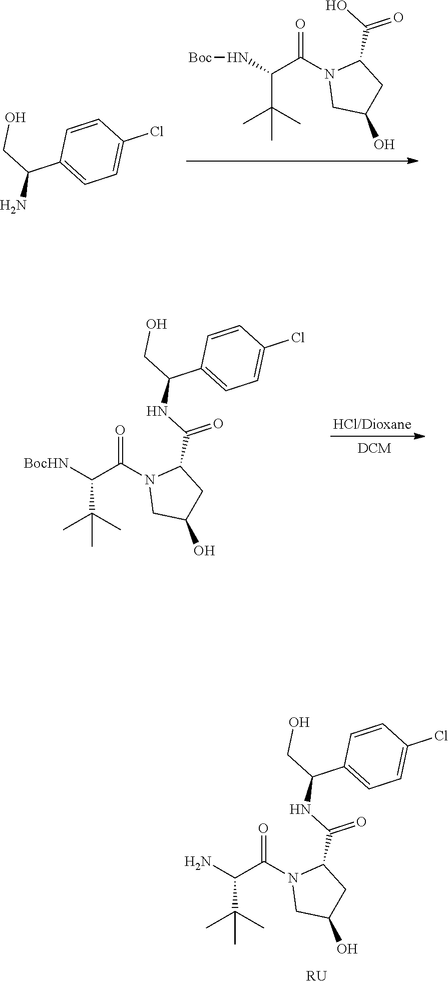 [SMILES: N[C@@H](CO)c1ccc(Cl)cc1 ; CC(C)(C)OC(=O)N[C@H](C(=O)N1C[C@H](O)C[C@H]1C(=O)O)C(C)(C)C ; CC(C)(C)OC(=O)N[C@H](C(=O)N1C[C@H](O)C[C@H]1C(=O)N[C@@H](CO)c1ccc(Cl)cc1)C(C)(C)C ; CC(C)(C)[C@H](N)C(=O)N1C[C@H](O)C[C@H]1C(=O)N[C@@H](CO)c1ccc(Cl)cc1 ; "RU"]

### Step 1—Tert-butyl ((S)-1-((2S,4R)-2-(((R)-1-(4-chlorophenyl)-2-hydroxyethyl)carbamoyl)-4-hydroxypyrrolidin-1-yl)-3,3-dimethyl-1-oxobutan-2-yl)carbamate

To a solution of (2S,4R)-1-((S)-2-((tert-butoxycarbonyl)amino)-3,3-dimethylbutanoyl)-4-hydroxypyrrolidine-2-carboxylic acid (403 mg, 1.17 mmol, CAS #1067658-27-8) in DMF (2 mL) was added HATU (578 mg, 1.52 mmol) and the mixture was stirred for 5 min. Then DIEA (756 mg, 5.85 mmol, 1.02 mL) and (R)-2-amino-2-(4-chlorophenyl)ethan-1-ol (200 mg, 1.17 mmol, CAS #630421-46-4) was added and the mixture was stirred at 25° C. for 0.5 hr. On completion, the reaction mixture was concentrated under reduced pressure to give a residue. The residue was purified by reversed-phase HPLC (0.1% FA condition) to give the title compound (490 mg, 75% yield, FA) as a white solid. LC-MS (ESI⁺) m/z 498.2 (M+H)⁺.

### Step 2—(2S,4R)-1-((S)-2-amino-3,3-dimethylbutanoyl)-N—((R)-1-(4-chlorophenyl)-2-hydroxyethyl)-4-hydroxypyrrolidine-2-carboxamide

To ((S)-1-((2S,4R)-2-(((R)-1-(4-chlorophenyl)-2-a solution of tert-butyl hydroxyethyl)carbamoyl)-4-hydroxypyrrolidin-1-yl)-3,3-dimethyl-1-oxobutan-2-yl)carbamate (490 mg, 900 μmol, FA) in DCM (10 mL) was added HCl/dioxane (4 M, 1 mL). The mixture was then stirred at 25° C. for 1 hr. On completion, the reaction mixture was concentrated under reduced pressure to give the title compound (360 mg) as yellow solid. LC-MS (ESI⁺) m/z 398.0 (M+H)⁺.

### Synthesis of 7-(((S)-1-((2S,4R)-2-(((R)-1-(4-chlorophenyl)-2-hydroxyethyl)carbamoyl)-4-hydroxypyrrolidin-1-yl)-3,3-dimethyl-1-oxobutan-2-yl)amino)-7-oxoheptanoic Acid (Intermediate RV)

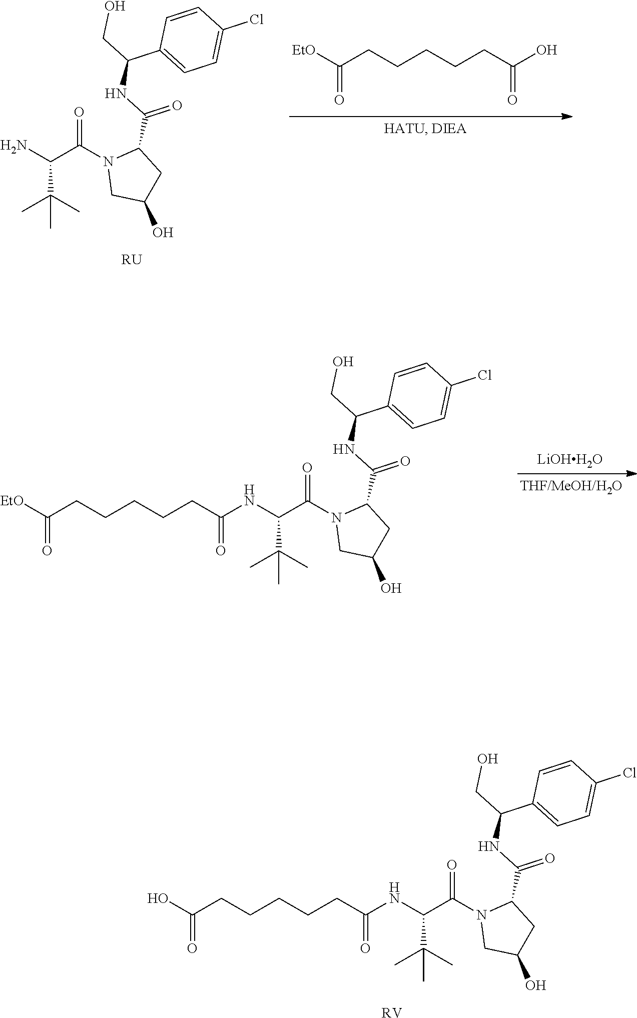

### Step 1—Ethyl 7-(((S)-1-((2S,4R)-2-(((R)-1-(4-chlorophenyl)-2-hydroxyethyl)carbamoyl)-4-hydroxypyrrolidin-1-yl)-3,3-dimethyl-1-oxobutan-2-yl)amino)-7-oxoheptanoate

To a solution of 7-ethoxy-7-oxoheptanoic acid (86.7 mg, 460 μmol, CAS #33018-91-6) in DMF (2 mL) was added HATU (227 mg, 598 μmol) and the mixture was stirred for 5 min. Then add DIEA (297 mg, 2.30 mmol, 401 uL) and (2S,4R)-1-((S)-2-amino-3,3-dimethylbutanoyl)-N—((R)-1-(4-chlorophenyl)-2-hydroxyethyl)-4-hydroxypyrrolidine-2-carboxamide (200 mg, 460 μmol, HCl, Intermediate RU) was added and the mixture was stirred at 25° C. for 0.5 hr. On completion, the reaction mixture was filtered and the filtrate was concentrated in vacuo to give the crude residue. The crude residue was purified by reversed-phase HPLC (0.8 g/L ammonium bicarbonate) to give the title compound (180 mg, 69% yield) as a colorless oil. LC-MS (ESI⁺) m/z 568.1 (M+H)⁺.

### Step 2—7-(((S)-1-((2S,4R)-2-(((R)-1-(4-chlorophenyl)-2-hydroxyethyl)carbamoyl)-4-hydroxypyrrolidin-1-yl)-3,3-dimethyl-1-oxobutan-2-yl)amino)-7-oxoheptanoic Acid

To a solution of ethyl 7-(((S)-1-((2S,4R)-2-(((R)-1-(4-chlorophenyl)-2-hydroxyethyl)carbamoyl)-4-hydroxypyrrolidin-1-yl)-3,3-dimethyl-1-oxobutan-2-yl)amino)-7-oxoheptanoate (680 mg, 1.52 mmol) in THF (20 mL) and H₂O (5 mL) was added dipotassium dioxido (dioxo) osmium dihydrate (39.2 mg, 106 μmol) and sodium periodate (1.63 g, 7.61 mmol, 421 uL). The mixture was then stirred at 25° C. for 2 hrs. On completion, the reaction mixture was diluted with water (10 mL) and extracted with ethyl acetate (2×10 mL). The combined organic layers were washed with brine (20 mL) and dried over anhydrous sodium sulfate, filtered and the filtrate was concentrated in vacuo to give the title compound (100 mg) as white solid. LC-MS (ESI⁺) m/z 540.4 (M+H)⁺.

### Synthesis of 7-(((S)-1-((2S,4R)-2-(((R)-1-(4-ethynylphenyl)-2-hydroxyethyl)carbamoyl)-4-hydroxypyrrolidin-1-yl)-3,3-dimethyl-1-oxobutan-2-yl)amino)-7-oxoheptanoic Acid (Intermediate RW)

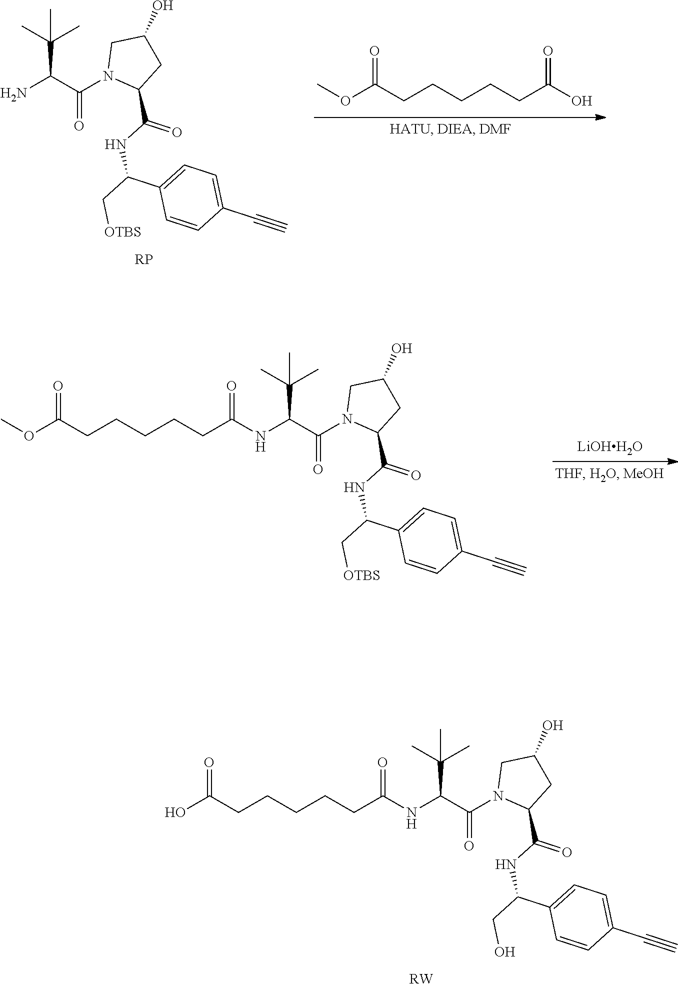 [SMILES: C#Cc1ccc([C@H](CO[Si](C)(C)C(C)(C)C)NC(=O)[C@@H]2C[C@@H](O)CN2C(=O)[C@@H](N)C(C)(C)C)cc1 ; COC(=O)CCCCCC(=O)O ; C#Cc1ccc([C@H](CO[Si](C)(C)C(C)(C)C)NC(=O)[C@@H]2C[C@@H](O)CN2C(=O)[C@@H](NC(=O)CCCCCC(=O)OC)C(C)(C)C)cc1 ; "LiOH·H2O" ; C#Cc1ccc([C@H](CO)NC(=O)[C@@H]2C[C@@H](O)CN2C(=O)[C@@H](NC(=O)CCCCCC(=O)O)C(C)(C)C)cc1 ; "RW"]

### Step 1—Methyl 7-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-7-oxoheptanoate

A solution of (2S,4R)-1-((S)-2-amino-3,3-dimethylbutanoyl)-N—((R)-2-((tert-butyldimethylsilyl)oxy)-1-(4-ethynylphenyl)ethyl)-4-hydroxypyrrolidine-2-carboxamide (1.00 g, 1.99 mmol, Intermediate RP), 7-methoxy-7-oxoheptanoic acid (562 mg, 2.99 mmol, CAS #33018-91-6), HATU (1.13 g, 2.99 mmol), and DIEA (772 mg, 5.97 mmol, 1.04 mL) in DMF (10 mL) was stirred at 0° C. for 20 min. On completion, the crude residue was purified by reversed-phase HPLC (0.8 g/L ammonium bicarbonate) to give the title compound (1.20 g) as a yellow solid. LC-MS (ESI⁺) m/z 694.3 (M+H)⁺.

### Step 2—7-(((S)-1-((2S,4R)-2-(((R)-1-(4-ethynylphenyl)-2-hydroxyethyl) carbamoyl)-4-hydroxypyrrolidin-1-yl)-3,3-dimethyl-1-oxobutan-2-yl)amino)-7-oxoheptanoic Acid

A solution of methyl 7-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-7-oxoheptanoate (300 mg, 446 μmol) and LiOH·H₂O (93.7 mg, 2.23 mmol) in THF (2 mL), MeOH (2 mL), H₂O (2 mL) was stirred at 25° C. for 12 hrs. On completion, to the reaction mixture was added HCl (1N) until the pH 6, then the mixture was concentrated in vacuo to give the residue. The crude residue was purified by reversed-phase HPLC (0.8 g/L ammonium bicarbonate) to give the title compound (120 mg) as a white solid. LC-MS (ESI⁺) m/z 530.2 (M+H)⁺.

### Synthesis of 6-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-6-oxohexanoic Acid (Intermediate RX)

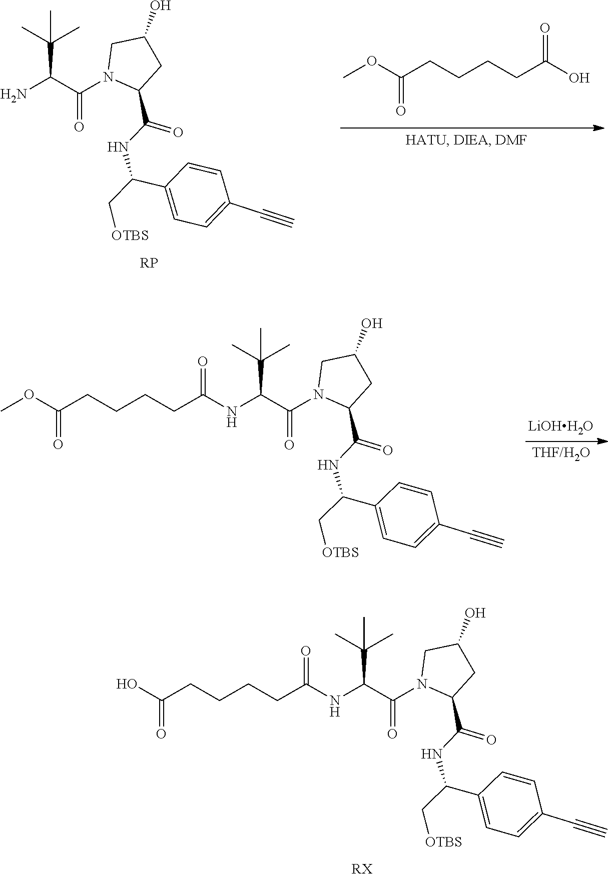

### Step 1—Methyl 6-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-6-oxohexanoate

To a solution (2S,4R)-1-[(2S)-2-amino-3,3-dimethyl-butanoyl]-N-[(1R)-2-[tert-butyl(dimethyl)silyl]oxy-1-(4-ethynylphenyl)ethyl]-4-hydroxy-pyrrolidine-2-carboxamide (300 mg, 597 μmol, Intermediate RP) in DMF (3 mL) and 6-methoxy-6-oxo-hexanoic acid (143 mg, 896 μmol, CAS #627-91-8) was added DIEA (386 mg, 2.99 mmol) and HATU (295 mg, 777 μmol). The mixture was then stirred at 0° C. for 0.5 hrs. On completion, the reaction mixture was diluted with water (20 mL) and extracted with ethyl acetate (3×30 mL). The combined organic layers were washed with brine (100 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 1/1) to give the title compound (140 mg, 36% yield) and (200 mg, 52% yield) as a yellow oil. LC-MS (ESI⁺) m/z 644.4 (M+H)⁺.

### Step 2—6-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-6-oxohexanoic Acid

To a solution of methyl 6-[[(1S)-1-[(2S,4R)-2-[[(1R)-2-[tert-butyl(dimethyl)silyl]oxy-1-(4-ethynylphenyl)ethyl]carbamoyl]-4-hydroxy-pyrrolidine-1-carbonyl]-2,2-dimethyl-propyl]amino]-6-oxohexanoate (140 mg, 217 μmol) in THF (4 mL) and H₂O (4 mL) was added LiOH·H₂O (91.2 mg, 2.17 mmol). The mixture was stirred at 25° C. for 1.5 hrs. On completion, to the reaction mixture was added HCl (1N) until the pH was 5, then concentrated in vacuo to give the title compound (90 mg) as a white solid. LC-MS (ESI⁺) m/z 538.4 (M+H)⁺.

### Synthesis of (2S,4R)-1-((S)-2-amino-3,3-dimethylbutanoyl)-N—((R)-1-(4-ethynylphenyl)-2-hydroxyethyl)-4-hydroxypyrrolidine-2-carboxamide (Intermediate RY)

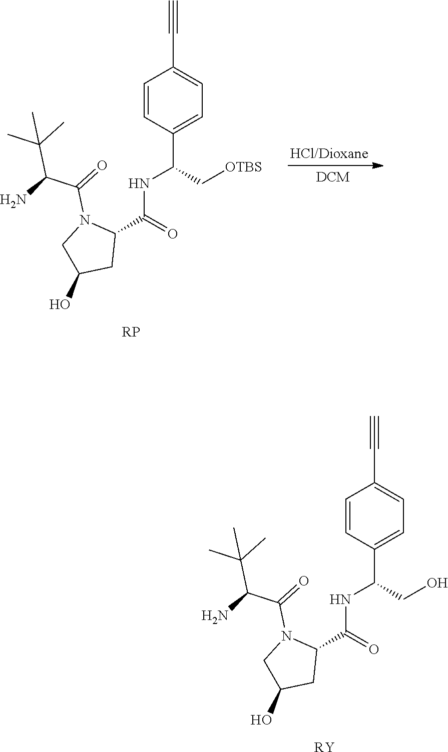

To a solution of (2S,4R)-1-((S)-2-amino-3,3-dimethylbutanoyl)-N—((R)-2-((tert-butyldimethylsilyl)oxy)-1-(4-ethynylphenyl)ethyl)-4-hydroxypyrrolidine-2-carboxamide (25 g, 41.54 mmol, Intermediate RP) in DCM (250 mL) was added HCl/dioxane (4 M, 50.00 mL). The mixture was then stirred at 0° C. for 2 hrs. On completion, the reaction mixture was concentrated under reduced pressure to give a residue. The crude residue was purified by reversed-phase HPLC (0.1% HCl condition) to give the title compound (11 g, 61% yield, HCl) as a yellow oil. LC-MS (ESI⁺) m/z 388.3 (M+H)⁺.

### Synthesis of 8-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)-8-oxooctanoic Acid (Intermediate RZ)

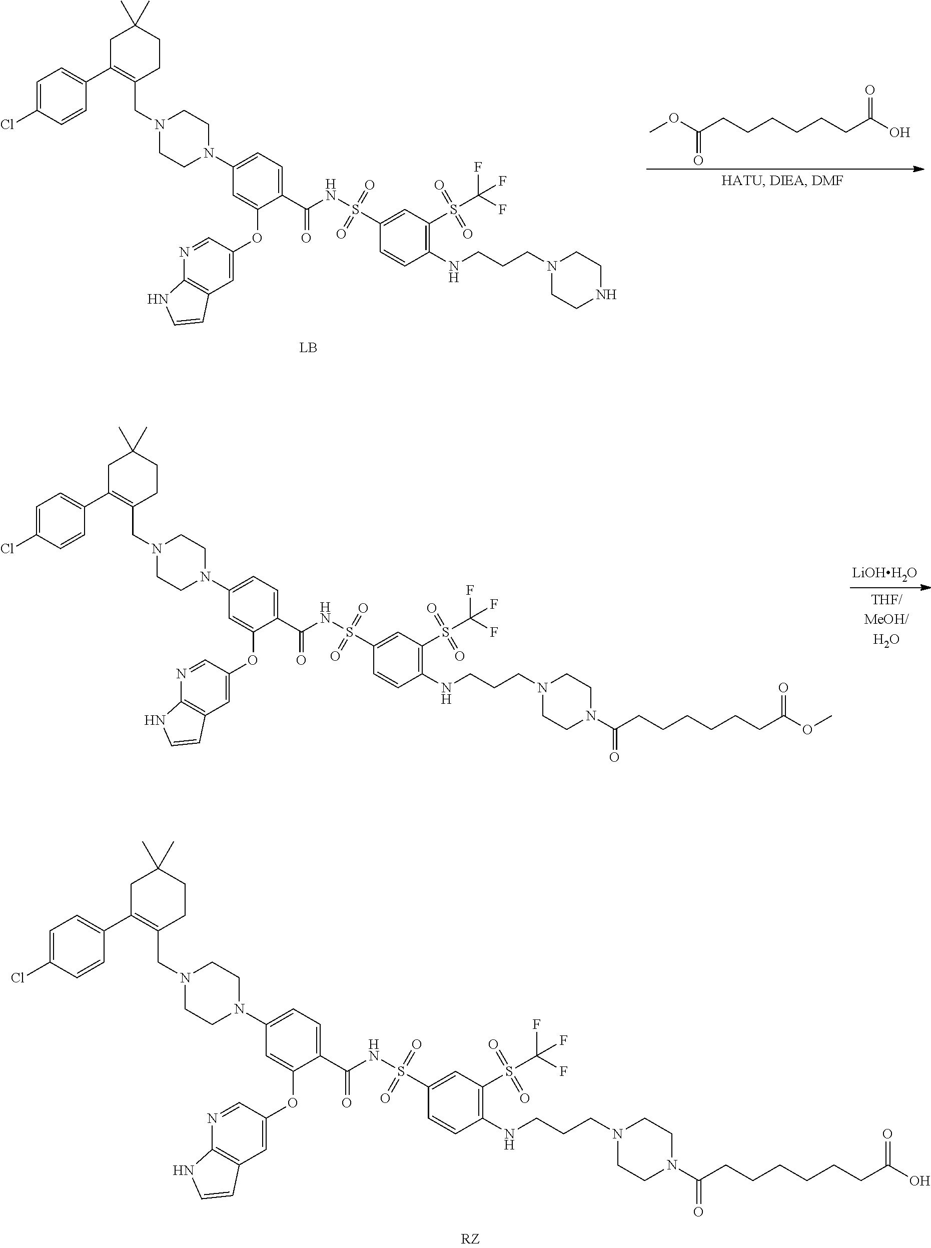 [SMILES: CC1(C)CCC(CN2CCN(c3ccc(C(=O)NS(=O)(=O)c4ccc(NCCCN5CCNCC5)c(S(=O)(=O)C(F)(F)F)c4)c(Oc4cnc5[nH]ccc5c4)c3)CC2)=C(c2ccc(Cl)cc2)C1 ; COC(=O)CCCCCCC(=O)O ; COC(=O)CCCCCCC(=O)N1CCN(CCCNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)CC1 ; "LiOH·H2O" ; CC1(C)CCC(CN2CCN(c3ccc(C(=O)NS(=O)(=O)c4ccc(NCCCN5CCN(C(=O)CCCCCCC(=O)O)CC5)c(S(=O)(=O)C(F)(F)F)c4)c(Oc4cnc5[nH]ccc5c4)c3)CC2)=C(c2ccc(Cl)cc2)C1]

### Step 1—Methyl 8-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)-8-oxooctanoate

To a solution of 8-methoxy-8-oxooctanoic acid (2 g, 10.6 mmol, CAS #3946-32-5) in DMF (40 mL) was added HATU (4.85 g, 12.8 mmol), DIEA (6.87 g, 53.1 mmol) and 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-(piperazin-1-yl)propyl)amino)-3-((trifluoromethyl)sulfonyl)phenyl)sulfonyl)benzamide (10.5 g, 10.6 mmol, Intermediate LB). The mixture was then stirred at 25° C. for 10 min. On completion, the reaction mixture was concentrated under reduced pressure to give a residue. The crude residue was purified by reversed-phase HPLC (0.8 g/L ammonium bicarbonate) to give the title compound (7.3 g, 56% yield) as a yellow solid. LC-MS (ESI⁺) m/z 557.6 (M/2+H)⁺.

### Step 2—8-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)-8-oxooctanoic Acid

To a solution of methyl 8-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)-8-oxooctanoate (7.3 g, 6.33 mmol) in THF (40 mL), MeOH (10 mL) and H₂O (10 mL) was added LiOH·H₂O (797 mg, 19.0 mmol). The mixture was then stirred at 25° C. for 2 hrs. On completion, the reaction mixture was concentrated under reduced pressure to give a residue. The crude residue was purified by reversed-phase HPLC (0.1% HCl condition) to give the title compound (7.3 g, 56% yield, HCl) as a yellow solid. LC-MS (ESI⁺) m/z 570.7 (M/2+H)⁺.

### Synthesis of Tert-butyl 4-((4-sulfamoyl-2-((trifluoromethyl)sulfonyl)phenyl)amino)piperidine-1-carboxylate (Intermediate SA)

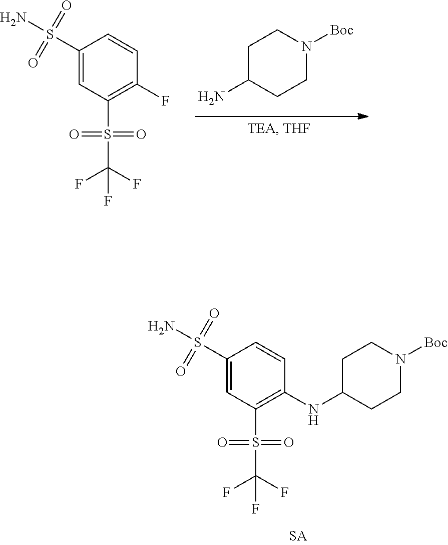 [SMILES: NS(=O)(=O)c1ccc(F)c(S(=O)(=O)C(F)(F)F)c1 ; CC(C)(C)OC(=O)N1CCC(N)CC1 ; CC(C)(C)OC(=O)N1CCC(Nc2ccc(S(N)(=O)=O)cc2S(=O)(=O)C(F)(F)F)CC1]

A solution of 4-fluoro-3-((trifluoromethyl)sulfonyl)benzenesulfonamide (1.00 g, 3.25 mmol, CAS #1027345-08-9), tert-butyl 4-aminopiperidine-1-carboxylate (652 mg, 3.25 mmol, 5.27 mL, CAS #502482-34-0), and TEA (988 mg, 9.76 mmol, 1.36 mL) in THF (10 mL) was stirred at 50° C. for 12 hrs. On completion, the reaction mixture was quenched with water (30 mL) and extracted by ethyl acetate/dichloromethane (3×20 mL). The combined organic layers were washed with brine (30 mL) and dried over anhydrous sodium sulfate, filtered and the filtrate was concentrated in vacuo to give the title compound (1.20 g) as a light yellow solid. ¹H NMR (400 MHz, DMSO-d₆) δ=8.03 (d, J=2.4 Hz, 1H), 7.97 (dd, J=2.0, 9.2 Hz, 1H), 7.41 (s, 2H), 7.34 (d, J=9.2 Hz, 1H), 6.70 (d, J=8.0 Hz, 1H), 3.87 (dd, J=2.4, 10.8 Hz, 3H), 2.96 (s, 2H), 1.91 (d, J=10.0 Hz, 2H), 1.40 (s, 9H), 1.38 (s, 2H).

### Synthesis of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(piperidin-4-ylamino)-3-((trifluoromethyl)sulfonyl)phenyl)sulfonyl)benzamide (Intermediate SB)

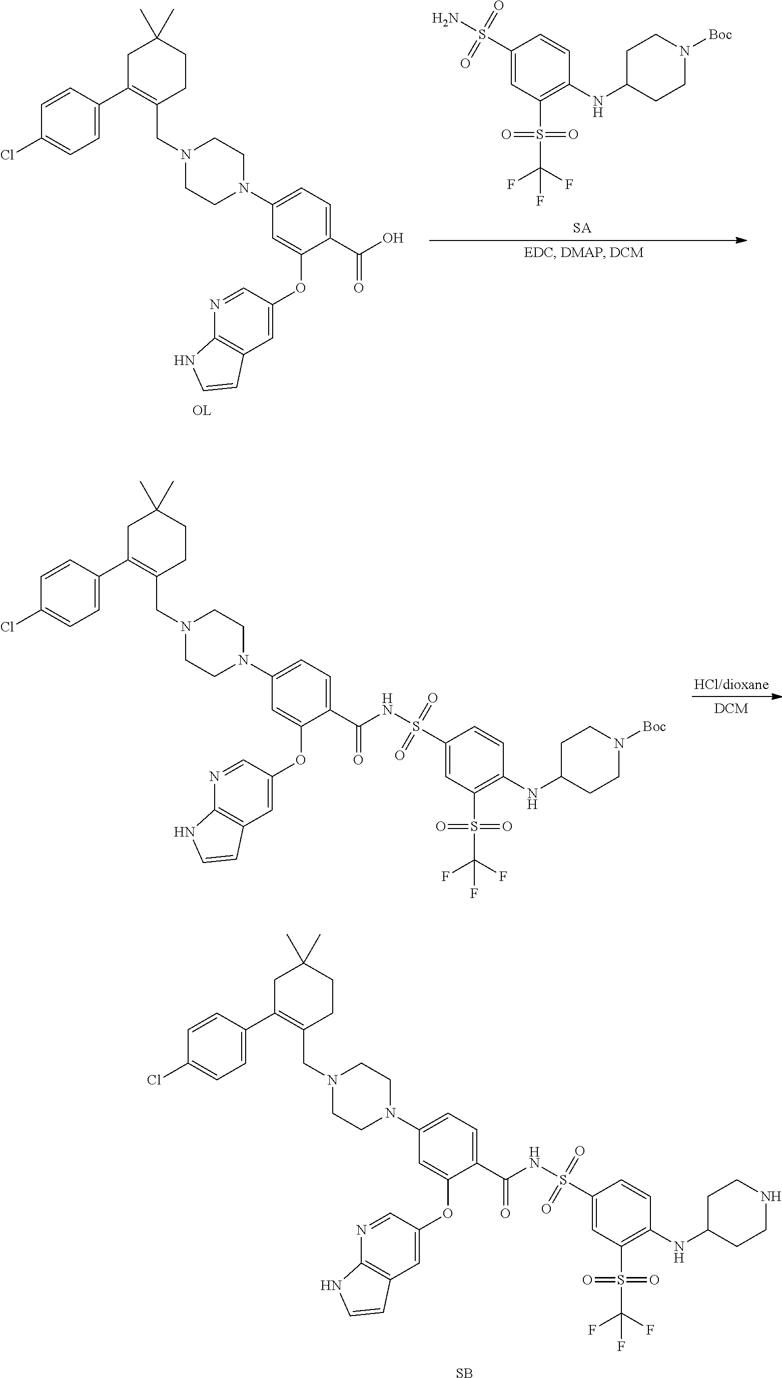 [SMILES: CC1(C)CCC(CN2CCN(c3ccc(C(=O)O)c(Oc4cnc5[nH]ccc5c4)c3)CC2)=C(c2ccc(Cl)cc2)C1 ; CC(C)(C)OC(=O)N1CCC(Nc2ccc(S(N)(=O)=O)cc2S(=O)(=O)C(F)(F)F)CC1 ; CC1(C)CCC(CN2CCN(c3ccc(C(=O)NS(=O)(=O)c4ccc(NC5CCN(C(=O)OC(C)(C)C)CC5)c(S(=O)(=O)C(F)(F)F)c4)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(NC5CCNCC5)c(S(=O)(=O)C(F)(F)F)c4)c(Oc4cnc5[nH]ccc5c4)c3)CC2)=C(c2ccc(Cl)cc2)C1 ; "SB"]

### Step 1—Tert-butyl 4-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)piperidine-1-carboxylate

A solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (500 mg, 875 μmol, Intermediate OL), tert-butyl 4-((4-sulfamoyl-2-((trifluoromethyl)sulfonyl)phenyl)amino)piperidine-1-carboxylate (427 mg, 875 μmol, Intermediate SA), DMAP (267 mg, 2.19 mmol), and EDC (272 mg, 1.75 mmol, 310 uL) in DCM (2 mL) was stirred at 25° C. for 1 hr. On completion, the crude residue was purified by reversed-phase HPLC (0.8 g/L ammonium bicarbonate) to give the title compound (668 mg) as a white solid. LCMS (ESI⁺) m/z 1040.5 (M+H)⁺.

### Step 2—2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(piperidin-4-ylamino)-3-((trifluoromethyl)sulfonyl)phenyl)sulfonyl)benzamide

A solution of tert-butyl 4-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)piperidine-1-carboxylate (400 mg, 384 μmol) in HCl/dioxane (2 mL) and DCM (3 mL) was then stirred at 25° C. for 2 hrs. On completion, the mixture was concentrated in vacuo to give the title compound (320 mg) as a yellow solid. LCMS (ESI⁺) m/z 940.5 (M+H)⁺.

### Synthesis of (2S,4R)-1-((S)-2-(7-(4-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)piperidin-1-yl) heptanamido)-3,3-dimethylbutanoyl)-N—((R)-2-((tert-butyldimethylsilyl)oxy)-1-(4-ethynylphenyl)ethyl)-4-hydroxypyrrolidine-2-carboxamide (Intermediate SC)

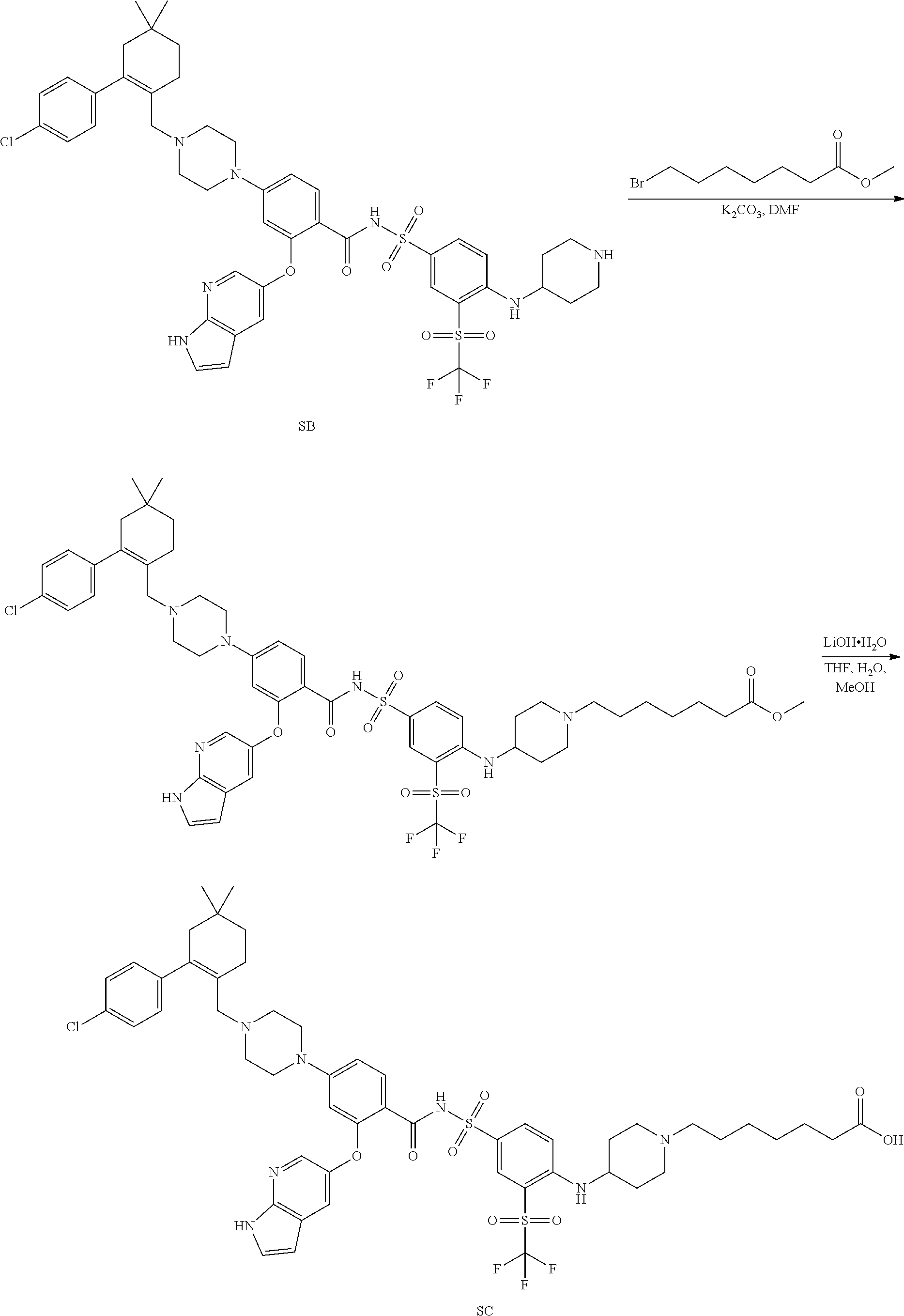

### Step 1—Methyl 7-(4-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)piperidin-1-yl)heptanoate

A solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(piperidin-4-ylamino)-3-((trifluoromethyl)sulfonyl)phenyl)sulfonyl)benzamide (100 mg, 106 μmol, Intermediate SB), methyl 7-bromoheptanoate (35.6 mg, 159 μmol, CAS #54049-24-0), K₂CO₃ (44.1 mg, 319 μmol) in DMF (1 mL) was stirred at 80° C. for 8 hrs. On completion, the crude residue was purified by reversed-phase HPLC (0.1% ammonium hydroxide) to give the title compound (100 mg) as a white solid. ¹H NMR (400 MHz, DMSO-d₆) δ=11.81-11.72 (m, 2H), 10.47-10.34 (m, 2H), 8.20 (d, J=2.4 Hz, 1H), 8.05 (d, J=2.4 Hz, 1H), 8.02-7.97 (m, 1H), 7.58-7.56 (m, 1H), 7.55-7.53 (m, 1H), 7.49 (d, J=8.8 Hz, 1H), 7.39 (d, J=8.4 Hz, 2H), 7.22 (d, J=9.6 Hz, 1H), 7.09 (d, J=8.4 Hz, 2H), 6.77-6.68 (m, 2H), 6.42 (dd, J=2.0, 3.6 Hz, 1H), 6.25 (d, J=2.0 Hz, 1H), 4.04-3.86 (m, 2H), 3.62 (d, J=13.2 Hz, 2H), 3.59 (s, 3H), 3.54 (d, J=4.0 Hz, 4H), 3.28-3.22 (m, 4H), 3.01 (d, J=11.2 Hz, 2H), 2.73-2.66 (m, 2H), 2.33-2.29 (m, 4H), 2.12 (d, J=12.4 Hz, 2H), 2.01 (s, 3H), 1.92-1.79 (m, 2H), 1.69 (dd, J=5.2, 6.8 Hz, 2H), 1.57-1.51 (m, 2H), 1.44 (t, J=6.0 Hz, 2H), 1.31 (d, J=4.0 Hz, 4H), 0.94 (s, 6H).

### Step 2—(2S,4R)-1-((S)-2-(7-(4-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)piperidin-1-yl) heptanamido)-3,3-dimethylbutanoyl)-N—((R)-2-((tert-butyldimethylsilyl)oxy)-1-(4-ethynylphenyl)ethyl)-4-hydroxypyrrolidine-2-carboxamide

A solution of methyl 7-(4-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)piperidin-1-yl)heptanoate (70.0 mg, 64.6 μmol) and LiOH·H₂O (13.6 mg, 323 μmol) in THF (0.2 mL), H₂O (0.2 mL), and MeOH (0.2 mL) was stirred at 0° C. for 2 hrs. On completion, the crude residue was purified by reversed-phase HPLC (0.8 g/L ammonium bicarbonate) to give the title compound (50.0 mg) as a white solid. LC-MS (ESI⁺) m/z 1068.2 (M+H)⁺.

### Synthesis of Tert-butyl 3-((4-sulfamoyl-2-((trifluoromethyl)sulfonyl)phenyl)amino)pyrrolidine-1-carboxylate (Intermediate SD)

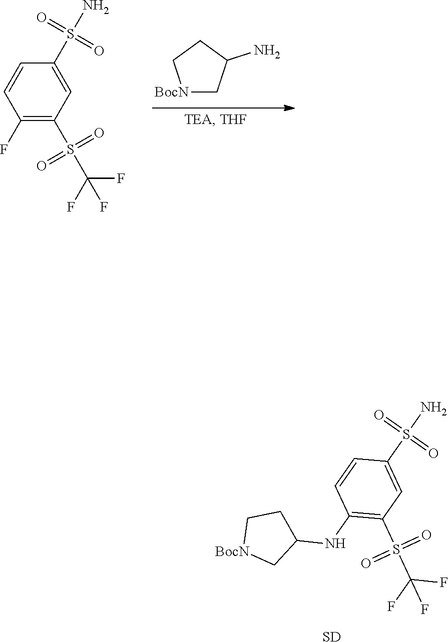 [SMILES: NS(=O)(=O)c1ccc(F)c(S(=O)(=O)C(F)(F)F)c1 ; CC(C)(C)OC(=O)N1CCC(N)C1 ; CC(C)(C)OC(=O)N1CCC(Nc2ccc(S(N)(=O)=O)cc2S(=O)(=O)C(F)(F)F)C1 ; "SD"]

To a solution of tert-butyl 3-aminopyrrolidine-1-carboxylate (2 g, 10.7 mmol, CAS #186550-13-0) in THF (30 mL) was added TEA (42.9 mmol, 5.98 mL) and 4-fluoro-3-(trifluoromethylsulfonyl)benzenesulfonamide (3.30 g, 10.7 mmol, CAS #1027345-08-9). The mixture was then stirred at 50° C. for 4 hrs. On completion, the reaction mixture was quenched by addition of H₂O (50 mL) and extracted with EA (50 mL×3). The combined organic layers were washed with brine (20 mL) and dried over anhydrous sodium sulfate, filtered and the filtrate was concentrated in vacuo to give the title compound (4 g) as yellow solid. LC-MS (ESI⁺) m/z 496.1 (M-Na)⁺.

### Synthesis of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(pyrrolidin-3-ylamino)-3-((trifluoromethyl)sulfonyl)phenyl)sulfonyl)benzamide (Intermediate SE)

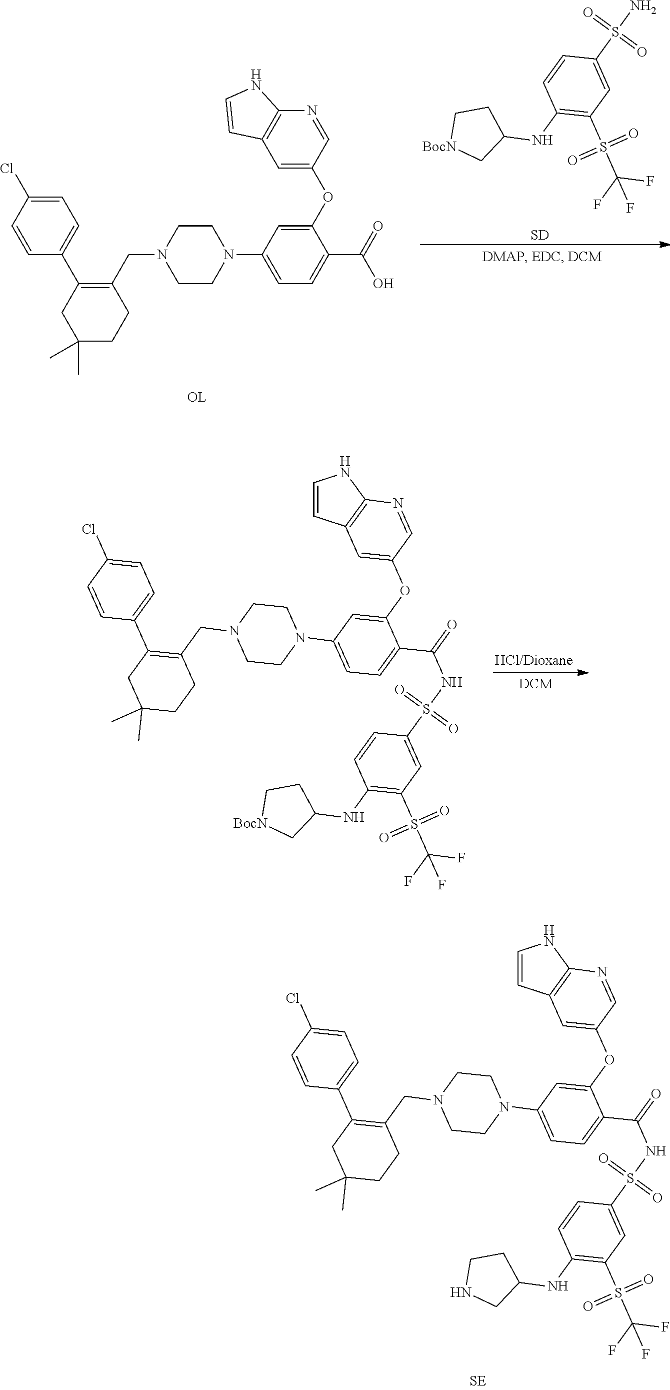 [SMILES: CC1(C)CCC(CN2CCN(c3ccc(C(=O)O)c(Oc4cnc5[nH]ccc5c4)c3)CC2)=C(c2ccc(Cl)cc2)C1 ; CC(C)(C)OC(=O)N1CCC(Nc2ccc(S(N)(=O)=O)cc2S(=O)(=O)C(F)(F)F)C1 ; "SD" ; CC1(C)CCC(CN2CCN(c3ccc(C(=O)NS(=O)(=O)c4ccc(NC5CCN(C(=O)OC(C)(C)C)C5)c(S(=O)(=O)C(F)(F)F)c4)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(NC5CCNC5)c(S(=O)(=O)C(F)(F)F)c4)c(Oc4cnc5[nH]ccc5c4)c3)CC2)=C(c2ccc(Cl)cc2)C1]

### Step 1—Tert-butyl 3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)pyrrolidine-1-carboxylate

To a solution of tert-butyl 3-[4-sulfamoyl-2-(trifluoromethylsulfonyl)anilino]pyrrolidine-1-carboxylate (300 mg, 633 μmol, Intermediate SD) in DCM (2 mL) was added DMAP (175 mg, 1.44 mmol), 4-[4-[[2-(4-chlorophenyl)-4,4-dimethyl-cyclohexen-1-yl]methyl]piperazin-1-yl]-2-(7H-pyrrolo[2,3-b]pyridin-5-yloxy)benzoic acid (328 mg, 576 μmol, Intermediate OL) and EDC (178 mg, 1.15 mmol, 203 uL). The mixture was then stirred at 30° C. for 4 hrs. The reaction mixture was filtered and the filtrate was concentrated in vacuo to give the crude residue. On completion, the crude product was purified by reversed-phase HPLC (0.1% FA condition) to give the title compound (340 mg, 56% yield, FA) as a white solid. LC-MS (ESI⁺) m/z 1026.3 (M+H)⁺.

### Step 2—2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(pyrrolidin-3-ylamino)-3-((trifluoromethyl)sulfonyl)phenyl)sulfonyl)benzamide

To a solution of tert-butyl 3-[4-[[4-[4-[[2-(4-chlorophenyl)-4,4-dimethyl-cyclohexen-1-yl]methyl]piperazin-1-yl]-2-(1H-pyrrolo[2,3-b]pyridin-5-yloxy)benzoyl]sulfamoyl]-2-(trifluoromethylsulfonyl)anilino]pyrrolidine-1-carboxylate (220 mg, 214 μmol) in DCM (10 mL) was added HCl/dioxane (4 M, 53.8 uL). The mixture was then stirred at 25° C. for 2 hrs. On completion, the reaction mixture was concentrated under reduced pressure to give the title compound (200 mg, HCl) as yellow oil. LC-MS (ESI⁺) m/z 926.1 (M+H)⁺.

### Synthesis of (9H-fluoren-9-yl)methyl 4-(3-((4-sulfamoyl-2-((trifluoromethyl)sulfonyl)phenyl)amino)cyclobutyl)piperazine-1-carboxylate (Intermediate SF)

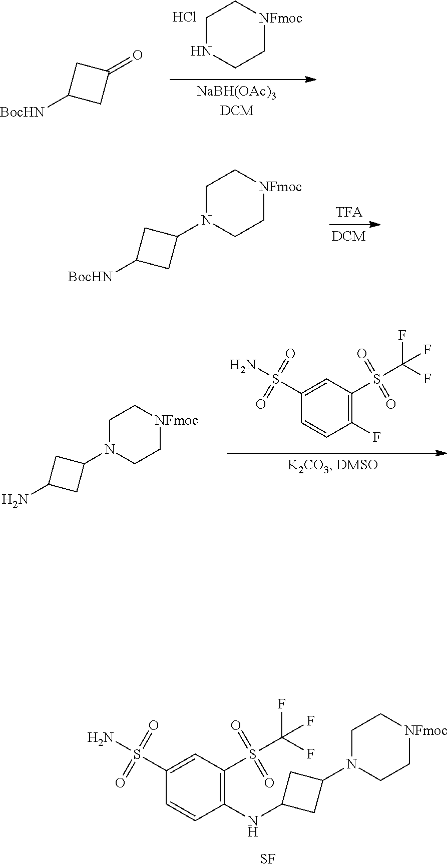

### Step 1—(9H-fluoren-9-yl)methyl 4-(3-((tert-butoxycarbonyl)amino)cyclobutyl)piperazine-1-carboxylate

To a solution of tert-butyl (3-oxocyclobutyl)carbamate (550 mg, 2.97 mmol, CAS #154748-49-9) and (9H-fluoren-9-yl)methyl piperazine-1-carboxylate hydrochloride (1.02 g, 2.97 mmol, CAS #215190-22-0) in DCM (20 mL) was added NaBH(OAc)₃ (1.26 g, 5.94 mmol). The mixture was then stirred at 25° C. for 2 hrs. On completion, the reaction mixture was quenched with water (20 mL) and extracted dichloromethane (2×20 mL). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 1/1) to give the title compound (1.2 g, 85% yield) as a white solid. LC-MS (ESI⁺) m/z 478.2 (M+H)⁺.

### Step 2—(9H-fluoren-9-yl)methyl 4-(3-aminocyclobutyl)piperazine-1-carboxylate

To a solution of (9H-fluoren-9-yl)methyl 4-(3-((tert-butoxycarbonyl)amino)cyclobutyl)piperazine-1-carboxylate (1.2 g, 2.51 mmol) in DCM (2 mL) was added TFA (9.24 g, 81.04 mmol, 6.00 mL). The mixture was then stirred at 25° C. for 1 hr. On completion, the reaction mixture was filtered and concentrated under reduced pressure to give the title compound (1.3 g, TFA) as yellow oil. LC-MS (ESI⁺) m/z 378.2 (M+H)⁺.

### Step 3—(9H-fluoren-9-yl)methyl 4-(3-((4-sulfamoyl-2-((trifluoromethyl)sulfonyl)phenyl)amino)cyclobutyl)piperazine-1-carboxylate

To a solution of (9H-fluoren-9-yl)methyl 4-(3-aminocyclobutyl)piperazine-1-carboxylate (0.9 g, 1.83 mmol, TFA) and 4-fluoro-3-((trifluoromethyl)sulfonyl)benzenesulfonamide (563 mg, 1.83 mmol, CAS #1027345-08-9) in DMSO (10 mL) was added K₂CO₃ (253 mg, 1.83 mmol). The mixture was then stirred at 80° C. for 5 hrs. On completion, the reaction mixture was quenched with water (10 mL) and extracted with ethyl acetate (2×10 mL). The combined organic layers were washed with brine (10 mL) and dried over anhydrous sodium sulfate, filtered and the filtrate was concentrated in vacuo to give the crude residue. The crude residue was purified by reversed-phase HPLC (0.8 g/L ammonium bicarbonate) to give the title compound (500 mg, 41% yield) as a white solid. LC-MS (ESI⁺) m/z 665.0 (M+H)⁺.

### Synthesis of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-(piperazin-1-yl)cyclobutyl)amino)-3-((trifluoromethyl)sulfonyl)phenyl)sulfonyl)benzamide (Intermediate SG)

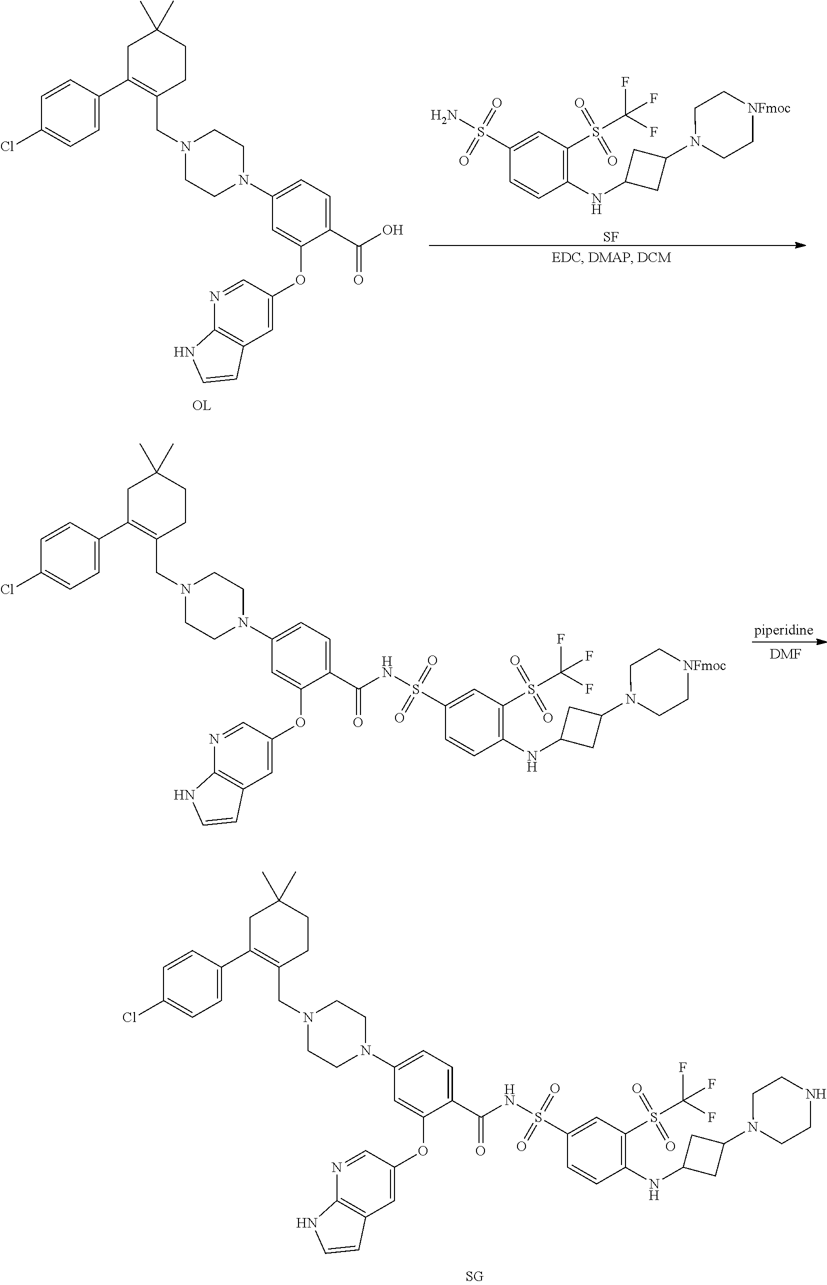

### Step 1—(9H-fluoren-9-yl)methyl 4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)cyclobutyl)piperazine-1-carboxylate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (172 mg, 300 μmol, Intermediate OL) in DCM (5 mL) was added DMAP (91.9 mg, 752 μmol) and the mixture was stirred for 5 min. Then add EDC (93.4 mg, 601 μmol, 106 uL) and (9H-fluoren-9-yl)methyl 4-(3-((4-sulfamoyl-2-((trifluoromethyl)sulfonyl)phenyl)amino)cyclobutyl)piperazine-1-carboxylate (200 mg, 300 μmol, Intermediate SF) was added and the mixture was stirred at 25° C. for 2 hrs. On completion, the reaction mixture was filtered and the filtrate was concentrated in vacuo to give the crude residue. The crude residue was purified by reversed-phase HPLC (0.8 g/L ammonium bicarbonate) to give the title compound (270 mg, 74% yield) as a white solid. LC-MS (ESI⁺) m/z 1217.0 (M+H)⁺.

### Step 2—2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-(piperazin-1-yl)cyclobutyl)amino)-3-((trifluoromethyl)sulfonyl)phenyl)sulfonyl)benzamide

To a solution of (9H-fluoren-9-yl)methyl 4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)cyclobutyl)piperazine-1-carboxylate (100 mg, 82.1 μmol) in DMF (2 mL) was added piperidine (6.99 mg, 82.1 μmol, 8.11 uL). The mixture was then stirred at 25° C. for 12 hrs. On completion, the reaction mixture was filtered and the filtrate was concentrated in vacuo to give the crude residue. The residue was purified by reversed-phase HPLC (0.8 g/L ammonium bicarbonate) to give the title compound (80 mg, 98% yield) as a yellow oil. LC-MS (ESI⁺) m/z 996.9 (M+H)⁺.

### Synthesis of 7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)heptanoic Acid (Intermediate SH)

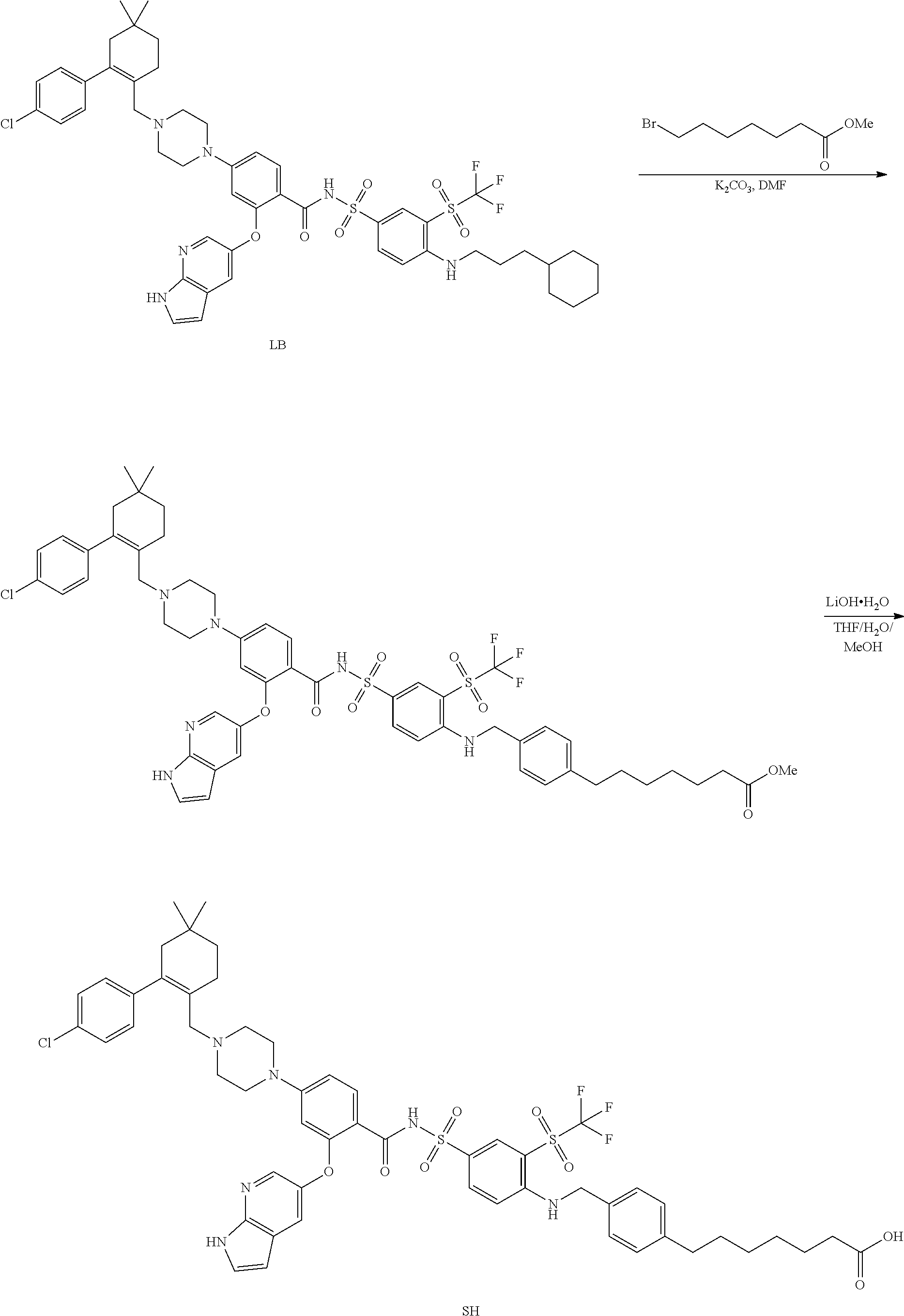

### Step 1—Methyl 7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)heptanoate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-(piperazin-1-yl)propyl)amino)-3-((trifluoromethyl)sulfonyl)phenyl)sulfonyl)benzamide (300 mg, 305 μmol, Intermediate LB) and methyl 7-bromoheptanoate (136 mg, 610 μmol, CAS #54049-24-0) in DMF (12 mL) was added KI (5.06 mg, 30.5 μmol) and K₂CO₃ (126 mg, 915 μmol). The mixture was then stirred at 80° C. for 12 hrs. On completion, the reaction mixture was concentrated under reduced pressure to give a residue. The crude residue was purified by reversed-phase HPLC (FA condition 0.1%) to give the title compound (115 mg, 32% yield) as a white solid. LC-MS (ESI⁺) m/z 563.7 (M+2H)⁺/2.

### Step 2—7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)heptanoic Acid

To a solution of methyl 7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)heptanoate (115 mg, 102 μmol) in THF (4 mL), H₂O (1 mL) and MeOH (1 mL) was added LiOH·H₂O (21.4 mg, 511 μmol). The mixture was then stirred at 25° C. for 1 hr. On completion, to the reaction mixture was added HCl (1 mol) until the pH was 7, then diluted with water (7 mL) and extracted with ethyl acetate (3×10 mL). The combined organic layers were dried over anhydrous sodium sulfate, filtered and the filtrate was concentrated in vacuo to give the title compound (100 mg, 88% yield) as a white solid. LC-MS (ESI⁺) m/z 1111.4 (M+H)⁺.

### Synthesis of Tert-butyl 3-(((4-sulfamoyl-2-((trifluoromethyl)sulfonyl)phenyl)amino)methyl) azetidine-1-carboxylate (Intermediate SI)

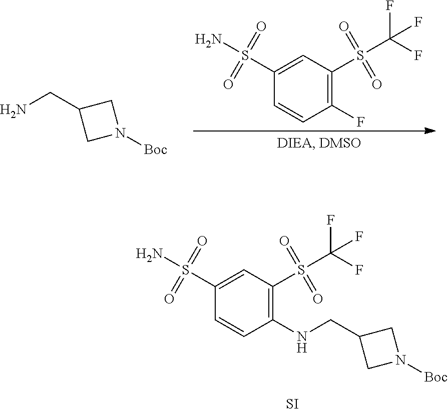 [SMILES: CC(C)(C)OC(=O)N1CC(CN)C1 ; NS(=O)(=O)c1ccc(F)c(S(=O)(=O)C(F)(F)F)c1 ; CC(C)(C)OC(=O)N1CC(CNc2ccc(S(N)(=O)=O)cc2S(=O)(=O)C(F)(F)F)C1 ; "SI"]

To a solution of tert-butyl 3-(aminomethyl)azetidine-1-carboxylate (1.1 g, 5.91 mmol, CAS #325775-44-8) and 4-fluoro-3-((trifluoromethyl)sulfonyl)benzenesulfonamide (1.81 g, 5.91 mmol, CAS #1027345-08-9) in DMSO (10 mL) was added DIEA (3.82 g, 29.53 mmol, 5.14 mL). The mixture was stirred at 90° C. for 2 hrs. On completion, the reaction mixture was quenched with water (10 mL) and extracted with ethyl acetate/dichloromethane (2×10 mL). The combined organic layers were washed by brine (20 mL) and dried over anhydrous sodium sulfate, filtered and the filtrate was concentrated in vacuo to give the title compound (2.8 g) as yellow solid. LC-MS (ESI⁺) m/z 496.1 (M+Na)⁺.

### Synthesis of 2-((1H-pyrrolo[2,3-b]pyridin-5-yl)oxy)-N-((4-((azetidin-3-ylmethyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide (Intermediate SJ)

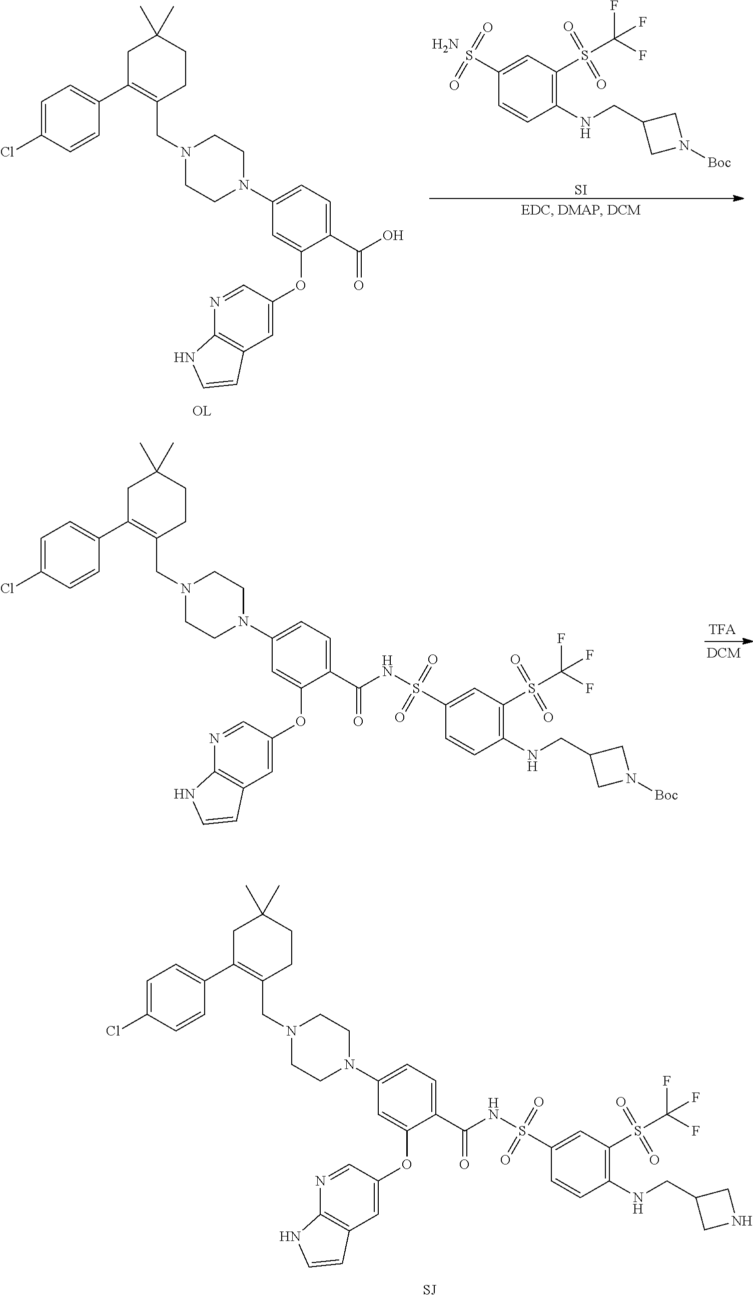

### Step 1—Tert-butyl 3-(((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)methyl)azetidine-1-carboxylate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (362 mg, 633 μmol, Intermediate OL) in DCM (5 mL) was added DMAP (193 mg, 1.58 mmol) and EDC (196 mg, 1.27 mmol, 224 uL) and the mixture was stirred for 5 min. Next, tert-butyl 3-(((4-sulfamoyl-2-((trifluoromethyl)sulfonyl)phenyl)amino)methyl)azetidine-1-carboxylate (300 mg, 633 μmol, Intermediate SI) was added and the mixture was stirred at 25° C. for 2 hrs. On completion, the reaction mixture was concentrated under reduced pressure to give a residue. The residue was purified by column chromatography (SiO₂, Petroleum ether:Ethyl acetate=1/0 to 0/1) to give the title compound (350 mg, 54% yield) as a white solid.

### Step 2—2-((1H-pyrrolo[2,3-b]pyridin-5-yl)oxy)-N-((4-((azetidin-3-ylmethyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide

To a solution of tert-butyl 3-(((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)methyl)azetidine-1-carboxylate (350 mg, 340 μmol) in DCM (10 mL) was added TFA (38.8 mg, 340 μmol, 25.2 uL). The mixture was then stirred at 25° C. for 1 hr. On completion, the reaction mixture was concentrated under reduced pressure to give the title compound (350 mg, TFA) as yellow oil LC-MS (ESI⁺) m/z 926.4 (M+H)⁺.

### Synthesis of 7-(3-(((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-(trifluoromethyl)sulfonyl)phenyl)amino)methyl)azetidin-1-yl)heptanoic Acid (Intermediate SK)

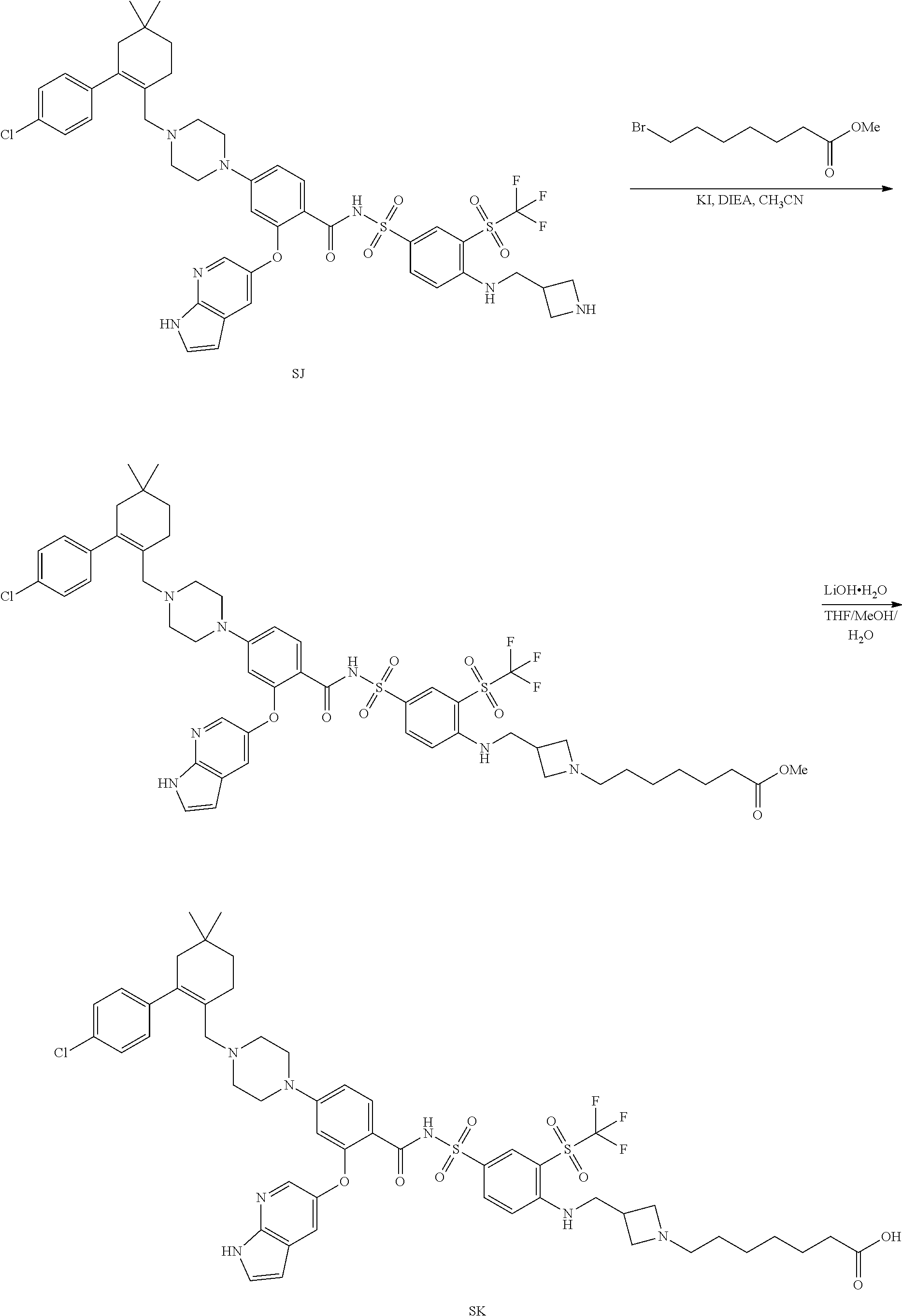 [SMILES: CC1(C)CCC(CN2CCN(c3ccc(C(=O)NS(=O)(=O)c4ccc(NCC5CNC5)c(S(=O)(=O)C(F)(F)F)c4)c(Oc4cnc5[nH]ccc5c4)c3)CC2)=C(c2ccc(Cl)cc2)C1 ; COC(=O)CCCCCCBr ; COC(=O)CCCCCCN1CC(CNc2ccc(S(=O)(=O)NC(=O)c3ccc(N4CCN(CC5=C(c6ccc(Cl)cc6)CC(C)(C)CC5)CC4)cc3Oc3cnc4[nH]ccc4c3)cc2S(=O)(=O)C(F)(F)F)C1 ; "LiOH·H2O" ; CC1(C)CCC(CN2CCN(c3ccc(C(=O)NS(=O)(=O)c4ccc(NCC5CN(CCCCCCC(=O)O)C5)c(S(=O)(=O)C(F)(F)F)c4)c(Oc4cnc5[nH]ccc5c4)c3)CC2)=C(c2ccc(Cl)cc2)C1 ; "SK"]

### Step 1—Methyl 7-(3-(((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)methyl)azetidin-1-yl)heptanoate

To a solution of 2-((1H-pyrrolo[2,3-b]pyridin-5-yl)oxy)-N-((4-((azetidin-3-ylmethyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide (300 mg, 288 μmol, TFA, Intermediate SJ) and methyl 7-bromoheptanoate (192 mg, 864 μmol, CAS #54049-24-0) in CH₃CN (2 mL) was added DIEA (186 mg, 1.44 mmol, 251 uL) and KI (4.79 mg, 28.8 μmol). The mixture was then stirred at 50° C. for 24 hrs. On completion, the reaction mixture was concentrated under reduced pressure to give the title compound (120 mg, 39% yield) as yellow oil. LC-MS (ESI⁺) m/z 534.9 (M/2)⁺.

### Step 2—7-(3-(((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)methyl)azetidin-1-yl)heptanoic Acid

To a solution of methyl 7-(3-(((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)methyl)azetidin-1-yl)heptanoate (120 mg, 112 μmol) in THF (4 mL), MeOH (1 mL) and H₂O (1 mL) was added LiOH·H₂O (14.1 mg, 337 μmol). The mixture was then stirred at 25° C. for 2 hrs. On completion, the mixture was concentrated in vacuo to give a residue. The crude residue was purified by reversed-phase HPLC (0.1% ammonium hydroxide) to give the title compound (80 mg, 28% yield) as a white solid. LC-MS (ESI⁺) m/z 1054.4 (M+H)⁺.

### Synthesis of Tert-butyl 4-((3S)-3-((4-sulfamoyl-2-((trifluoromethyl)sulfonyl)phenyl)amino)cyclopentyl)piperazine-1-carboxylate (Intermediate SL)

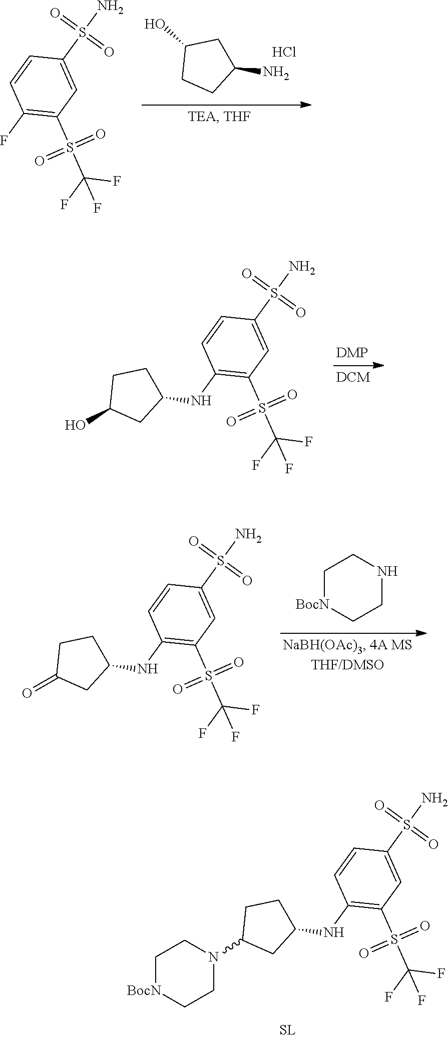

### Step 1—4-(((1S,3S)-3-hydroxycyclopentyl)amino)-3-((trifluoromethyl)sulfonyl)benzenesulfonamide

To a solution of (1S,3S)-3-aminocyclopentan-1-ol hydrochloride (985 mg, 7.16 mmol, HCl, CAS #1523530-42-8) in THF (20 mL) was added 4-fluoro-3-((trifluoromethyl)sulfonyl)benzenesulfonamide (2 g, 6.51 mmol, CAS #1027345-08-9) and TEA (2.63 g, 26.0 mmol, 3.62 mL). The mixture was then stirred at 50° C. for 12 hrs. On completion, the mixture was concentrated to give the title compound (2.6 g) as a white solid. LC-MS (ESI⁺) m/z 389.0 (M+H)⁺.

### Step 2—(S)-4-((3-oxocyclopentyl)amino)-3-((trifluoromethyl)sulfonyl)benzenesulfonamide

To a solution of 4-(((1S,3S)-3-hydroxycyclopentyl)amino)-3-((trifluoromethyl)sulfonyl)benzenesulfonamide (2.6 g, 6.69 mmol) in DCM (20 mL) and THF (10 mL) was added DMP (3.98 g, 9.37 mmol, 2.90 mL). The mixture was stirred at 0° C. for 3 hrs. On completion, the mixture was diluted with DCM (30 mL), quenched with Na₂S20; solution (100 mL) and separated. The organic layer was washed with NaHCO₃ solution (20 mL×3) then concentrated to give the title compound (2.6 g, HCl) as an off-white solid. LC-MS (ESI⁺) m/z 387.2 (M+H)⁺.

### Step 3—Tert-butyl 4-((3S)-3-((4-sulfamoyl-2-((trifluoromethyl)sulfonyl)phenyl)amino)cyclopentyl)piperazine-1-carboxylate

To a solution of (S)-4-((3-oxocyclopentyl)amino)-3-((trifluoromethyl)sulfonyl)benzenesulfonamide (2.2 g, 5.69 mmol) in THF (20 mL) and DMSO (5 mL) was added tert-butyl piperazine-1-carboxylate (1.27 g, 6.83 mmol, CAS #57260-71-6). HCOOH (274 mg, 5.69 mmol) and 4 Å molecular sieves (1 g). The mixture was stirred at 25° C. for 2 hours, then NaBH(OAc) 3 (3.02 g, 14.2 mmol) was added and the mixture was stirred at 25° C. for 3 hrs. On completion, the mixture was filtered to give a filtrate, diluted with EtOAc (50 mL), washed with brine (30 mL×3), dried over anhydrous Na₂SO₄, filtered to give a filtrate and concentrated to give a residue. The residue was purified by column chromatography (SiO₂, Petroleum ether/Ethyl acetate=2/1 to DCM:MeOH=30:1) to give the title compound (800 mg, 25% yield) as a brown solid. LC-MS (ESI⁺) m/z 557.2 (M+H)⁺.

### Synthesis of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(((1S)-3-(piperazin-1-yl)cyclopentyl)amino)-3-((trifluoromethyl)sulfonyl)phenyl)sulfonyl)benzamide (Intermediate SM)

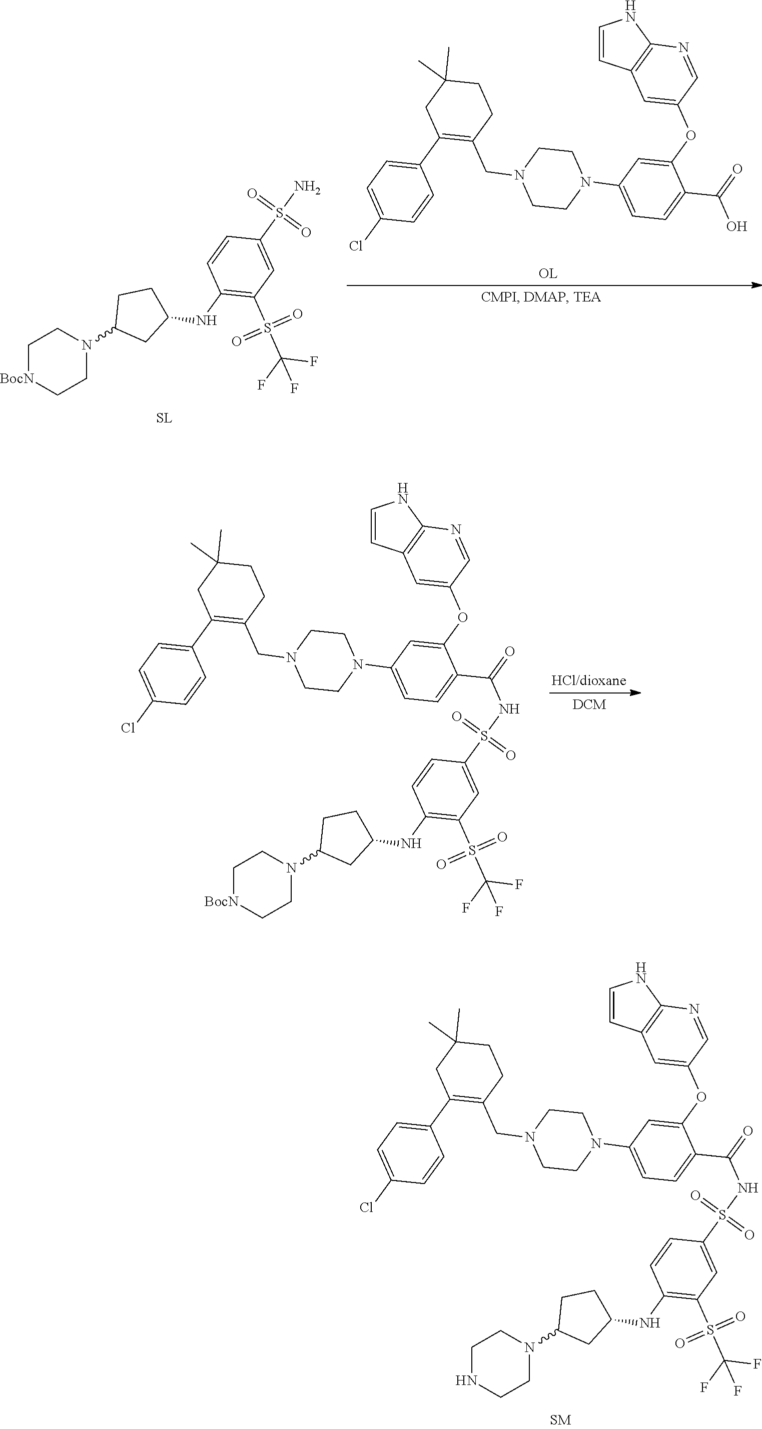 [SMILES: CC(C)(C)OC(=O)N1CCN(C2CC[C@H](Nc3ccc(S(N)(=O)=O)cc3S(=O)(=O)C(F)(F)F)C2)CC1 ; CC1(C)CCC(CN2CCN(c3ccc(C(=O)O)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(N[C@H]5CCC(N6CCN(C(=O)OC(C)(C)C)CC6)C5)c(S(=O)(=O)C(F)(F)F)c4)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(N[C@H]5CCC(N6CCNCC6)C5)c(S(=O)(=O)C(F)(F)F)c4)c(Oc4cnc5[nH]ccc5c4)c3)CC2)=C(c2ccc(Cl)cc2)C1]

### Step 1—Tert-butyl 4-((3S)-3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)cyclopentyl)piperazine-1-carboxylate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (431 mg, 755 μmol, Intermediate OL) in DCM (10 mL) was added TEA (115 mg, 1.13 mmol, 158 uL), DMAP (92.2 mg, 755 μmol) and CMPI (289 mg, 1.13 mmol). Then tert-butyl 4-((3S)-3-((4-sulfamoyl-2-((trifluoromethyl)sulfonyl)phenyl)amino)cyclopentyl)piperazine-1-carboxylate (420 mg, 755 μmol, Intermediate SL) was added and the mixture was stirred at 25° C. for 1 hr. On completion, the mixture was concentrated to give a residue and purified by column chromatography (SiO₂, Petroleum ether/Ethyl acetate=1/1 to DCM:MeOH=15:1) to give the title compound (800 mg, 73% yield) as a yellow solid. LC-MS (ESI⁺) m/z 1109.2 (M+H)⁺.

### Step 2—2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(((1S)-3-(piperazin-1-yl)cyclopentyl)amino)-3-((trifluoromethyl)sulfonyl)phenyl)sulfonyl)benzamide

To a solution of tert-butyl 4-((3S)-3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)cyclopentyl)piperazine-1-carboxylate (800 mg, 721 μmol) in DCM (12 mL) was added HCl/dioxane (4 M, 5.33 mL). The mixture was then stirred at 25° C. for 1 hr. On completion, the mixture was concentrated to give the title compound (700 mgHCl) as a yellow solid. LC-MS (ESI⁺) m/z 1009.5 (M+H)⁺.

### Synthesis of 2-((1H-pyrrolo[2,3-b]pyridin-5-yl)oxy)-N-((4-((3-((1R,4R)-2,5-diazabicyclo[2.2.1]heptan-2-yl)propyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide (Intermediate SN)

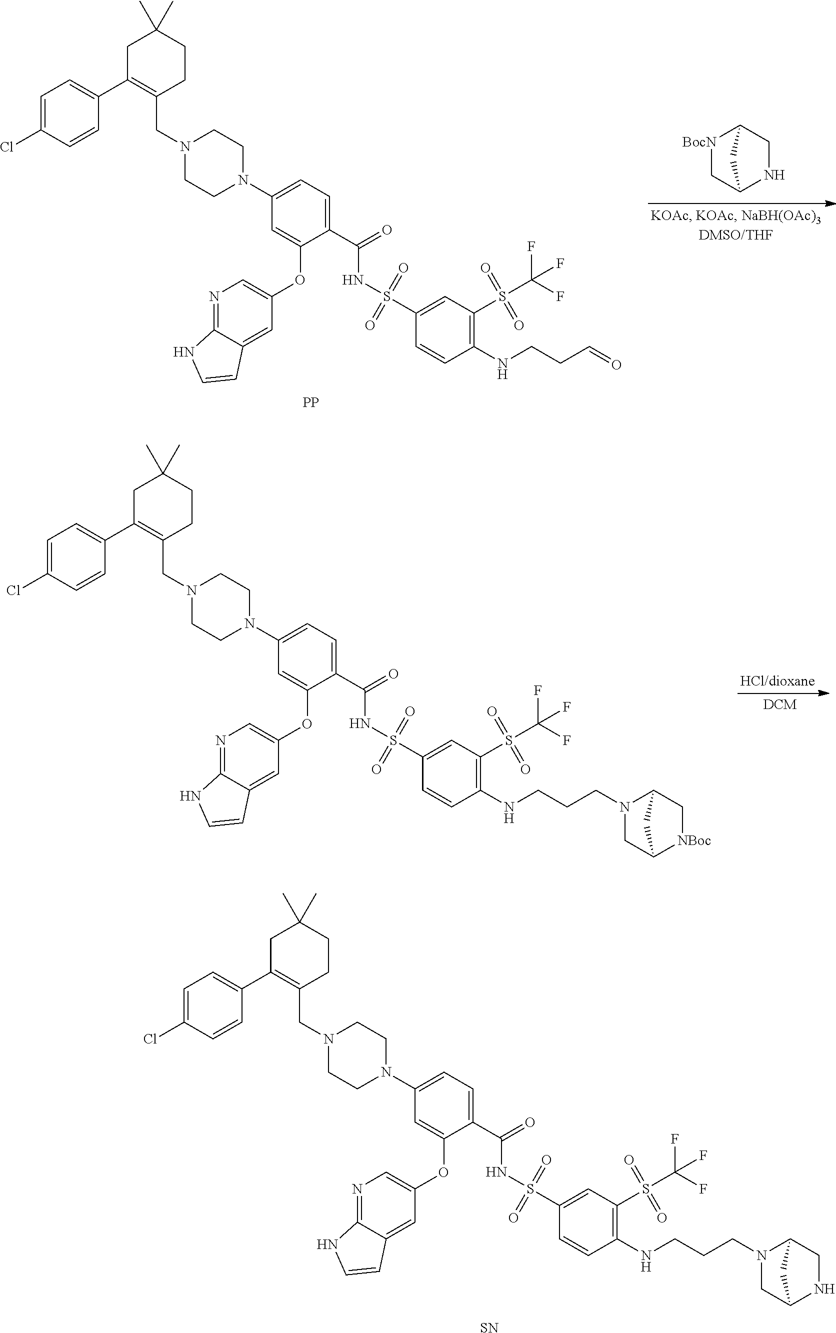 [SMILES: CC1(C)CCC(CN2CCN(c3ccc(C(=O)NS(=O)(=O)c4ccc(NCCC=O)c(S(=O)(=O)C(F)(F)F)c4)c(Oc4cnc5[nH]ccc5c4)c3)CC2)=C(c2ccc(Cl)cc2)C1 ; CC(C)(C)OC(=O)N1C[C@H]2C[C@@H]1CN2 ; "PP" ; CC1(C)CCC(CN2CCN(c3ccc(C(=O)NS(=O)(=O)c4ccc(NCCCN5C[C@H]6C[C@@H]5CN6C(=O)OC(C)(C)C)c(S(=O)(=O)C(F)(F)F)c4)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(NCCCN5C[C@H]6C[C@@H]5CN6)c(S(=O)(=O)C(F)(F)F)c4)c(Oc4cnc5[nH]ccc5c4)c3)CC2)=C(c2ccc(Cl)cc2)C1 ; "SN"]

### Step 1—Tert-butyl (1R,4R)-5-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)-2,5-diazabicyclo[2.2.1]heptane-2-carboxylate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-oxopropyl)amino)-3-((trifluoromethyl)sulfonyl)phenyl)sulfonyl)benzamide (520 mg, 569 μmol, Intermediate PP) tert-butyl (1R,4R)-2,5-diazabicyclo[2.2.1]heptane-2-carboxylate (169 mg, 854 μmol, CAS #113451-59-5) in THF (3 mL) and DMSO (1 mL) was added dropwise HOAc (171 mg, 2.85 mmol) and KOAc (168 mg, 1.71 mmol) at 25° C. over 1 hr. After addition, the mixture was stirred at this temperature for 1 hr, and then NaBH(OAc)₃ (362 mg, 1.71 mmol) was added dropwise at 0° C. The resulting mixture was stirred at 25° C. for 1 hr. On completion, the reaction mixture was concentrated under reduced pressure to give a residue. The crude residue was purified by reversed-phase HPLC (0.1% FA condition) to give the title compound (330 mg, 51% yield) as a white solid. ¹H NMR (400 MHz, DMSO-d₆) δ=11.62 (s, 1H), 8.08 (s, 1H), 7.98 (s, 1H), 7.85 (d, J=8.0 Hz, 1H), 7.51 (d, J=8.8 Hz, 1H), 7.47 (d, J=2.8 Hz, 1H), 7.43 (s, 1H), 7.34 (d, J=8.4 Hz, 3H), 7.04 (d, J=8.4 Hz, 2H), 6.90 (s, 1H), 6.65 (d, J=9.2 Hz, 1H), 6.36 (s, 1H), 6.21 (s, 1H), 4.23-4.17 (m, 1H), 3.03 (s, 5H), 2.73 (s, 2H), 2.54 (s, 1H), 2.21-2.11 (m, 8H), 1.95 (s, 2H), 1.70 (s, 3H), 1.39 (s, 17H), 0.92 (s, 7H). LC-MS (ESI⁺) m/z 1095.5 (M+H)⁺.

### Step 2—2-((1H-pyrrolo[2,3-b]pyridin-5-yl)oxy)-N-((4-((3-((1R,4R)-2,5-diazabicyclo[2.2.1]heptan-2-yl)propyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide

To a solution of tert-butyl (1R,4R)-5-[3-[4-[[4-[4-[[2-(4-chlorophenyl)-4,4-dimethyl-cyclohexen-1-yl]methyl]piperazin-1-yl]-2-(1H-pyrrolo[2,3-b]pyridin-5-yloxy)benzoyl]sulfamoyl]-2-(trifluoromethylsulfonyl)anilino]propyl]-2,5-diazabicyclo[2.2.1]heptane-2-carboxylate (330 mg, 301 μmol) in DCM (8 mL) was added HCl/dioxane (4 M, 2 mL). The mixture was then stirred at 25° C. for 0.5 hr. On completion, the reaction mixture was concentrated under reduced pressure to give the title compound (320 mg) as a white solid. LC-MS (ESI⁺) m/z 498.5 (M+2H)⁺/2.

### Synthesis of 2-((1H-pyrrolo[2,3-b]pyridin-5-yl)oxy)-N-((4-((3-((1S,4S)-2,5-diazabicyclo[2.2.1]heptan-2-yl)propyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide (Intermediate SO)

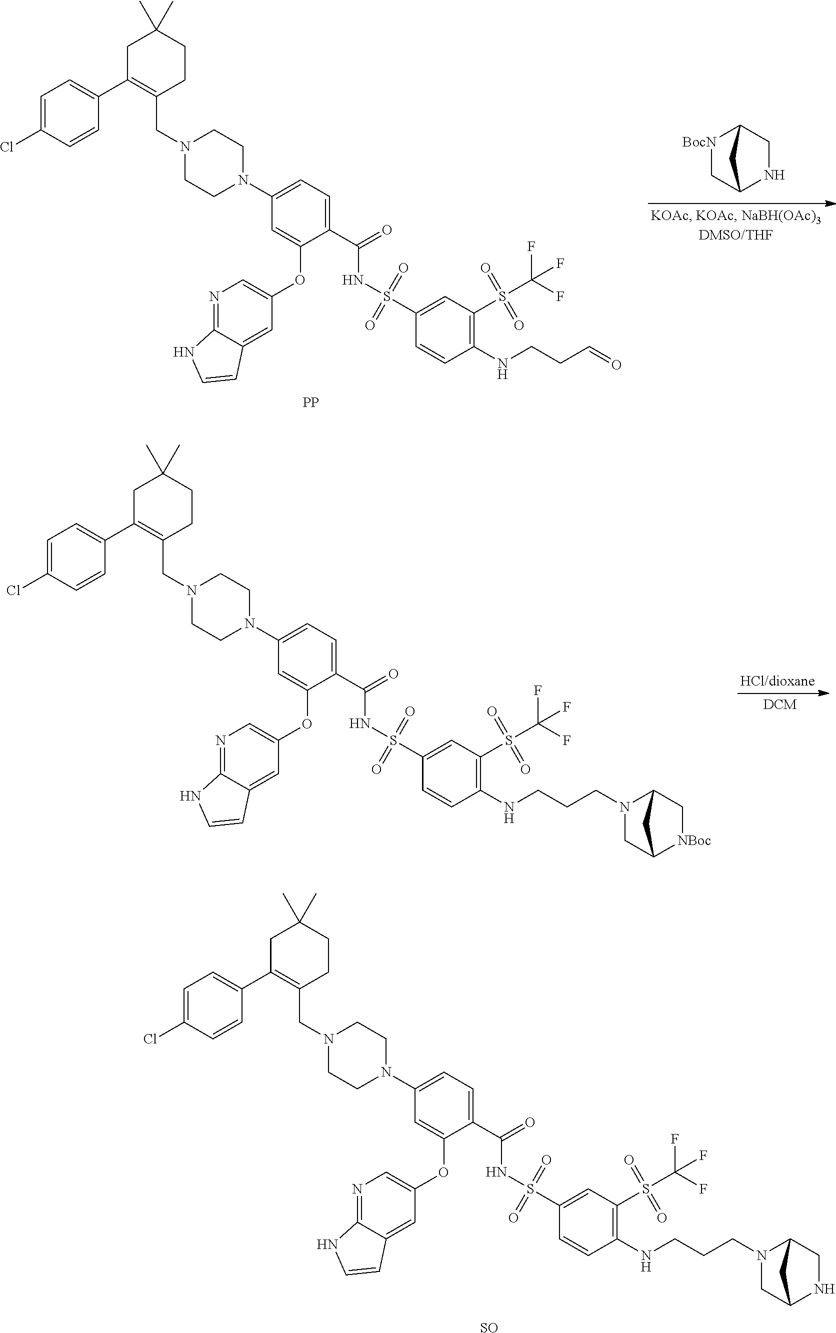 [SMILES: CC1(C)CCC(CN2CCN(c3ccc(C(=O)NS(=O)(=O)c4ccc(NCCC=O)c(S(=O)(=O)C(F)(F)F)c4)c(Oc4cnc5[nH]ccc5c4)c3)CC2)=C(c2ccc(Cl)cc2)C1 ; CC(C)(C)OC(=O)N1C[C@@H]2C[C@H]1CN2 ; "PP" ; CC1(C)CCC(CN2CCN(c3ccc(C(=O)NS(=O)(=O)c4ccc(NCCCN5C[C@@H]6C[C@H]5CN6C(=O)OC(C)(C)C)c(S(=O)(=O)C(F)(F)F)c4)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(NCCCN5C[C@@H]6C[C@H]5CN6)c(S(=O)(=O)C(F)(F)F)c4)c(Oc4cnc5[nH]ccc5c4)c3)CC2)=C(c2ccc(Cl)cc2)C1 ; "SO"]

### Step 1—Tert-butyl (1S,4S)-5-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)-2,5-diazabicyclo[2.2.1]heptane-2-carboxylate

To a solution of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-((3-oxopropyl)amino)-3-((trifluoromethyl)sulfonyl)phenyl)sulfonyl)benzamide (250 mg, 273 μmol, Intermediate PP) in DMSO (4 mL) was added dropwise HOAc (49.3 mg, 0.821 mmol), tert-butyl (1S,4S)-2,5-diazabicyclo[2.2.1]heptane-2-carboxylate (108 mg, 547 μmol, CAS #113451-59-5) and NaBH(OAc) 3 (145 mg, 0.684 mmol). Then the mixture was stirred at 25° C. for 2 hrs. On completion, the reaction mixture was concentrated under reduced pressure to give a residue. The crude residue was purified by reversed-phase HPLC (0.1% FA condition) to give the title compound (330 mg, 51% yield) as a white solid. LC-MS (ESI+) m/z 1095.6 (M+H)⁺.

### Step 2—2-((1H-pyrrolo[2,3-b]pyridin-5-yl)oxy)-N-((4-((3-((1S,4S)-2,5diazabicyclo[2.2.1]heptan-2-yl)propyl)amino)-3-((trifluoromethyl)sulfonyl)phenyl)sulfonyl)-4-(4-((4′-chloro-5,5-dimethyl-3,4,5,6-tetrahydro-[1,1′-biphenyl]-2-yl)methyl)piperazin-1-yl)benzamide

To a solution of tert-butyl (1S,4S)-5-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)-2,5-diazabicyclo[2.2.1]heptane-2-carboxylate (200 mg, 182 μmol) in DCM (4 mL) was added HCl/dioxane (4 M, 2 mL). The mixture was stirred at 25° C. for 0.5 hr. On completion, the reaction mixture was concentrated under reduced pressure to give the title compound (320 mg, crude) as a white solid. LC-MS (ESI+) m/z 995.3 (M+H)⁺.

### Synthesis of 4-((1-Methylpiperidin-4-yl)amino)-3-((trifluoromethyl)sulfonyl)benzenesulfonamide (Intermediate SP)

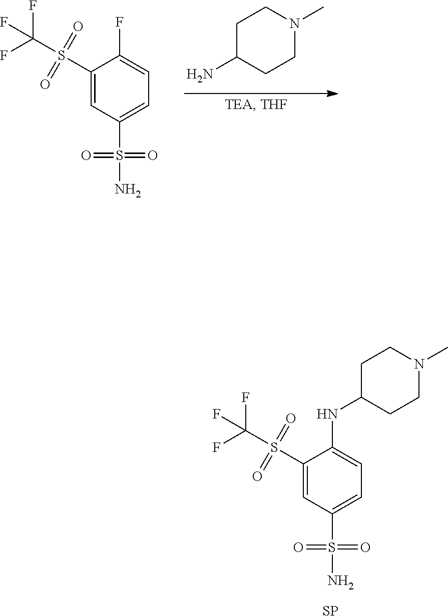

A solution of 4-fluoro-3-((trifluoromethyl)sulfonyl)benzenesulfonamide (500 mg, 1.63 mmol, CAS #1027345-08-9), 1-methylpiperidin-4-amine (186 mg, 1.63 mmol, CAS #41838-46-4), and TEA (494 mg, 4.88 mmol, 679 uL) in THF (5 mL) was stirred at 50° C. for 2 hrs. On completion, the mixture was concentrated in vacuo to give the title compound (530 mg) as a white solid. ¹H NMR (400 MHz, DMSO-d₆) δ=8.03 (d, J=2.0 Hz, 1H), 7.97 (dd, J=2.0, 9.2 Hz, 1H), 7.41 (s, 2H), 7.29 (d, J=9.2 Hz, 1H), 6.69 (d, J=7.6 Hz, 1H), 3.75-3.70 (m, 1H), 2.61 (d, J=9.6 Hz, 2H), 2.23-2.14 (m, 5H), 1.96-1.88 (m, 2H), 1.57-1.44 (m, 2H).

### Synthesis of (R)-2-((1H-pyrrolo[2,3-b]pyridin-5-yl)oxy)-4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-((1-methylpiperidin-4-yl)amino)-3-((trifluoromethyl)sulfonyl)phenyl)sulfonyl)benzamide (Intermediate SO)

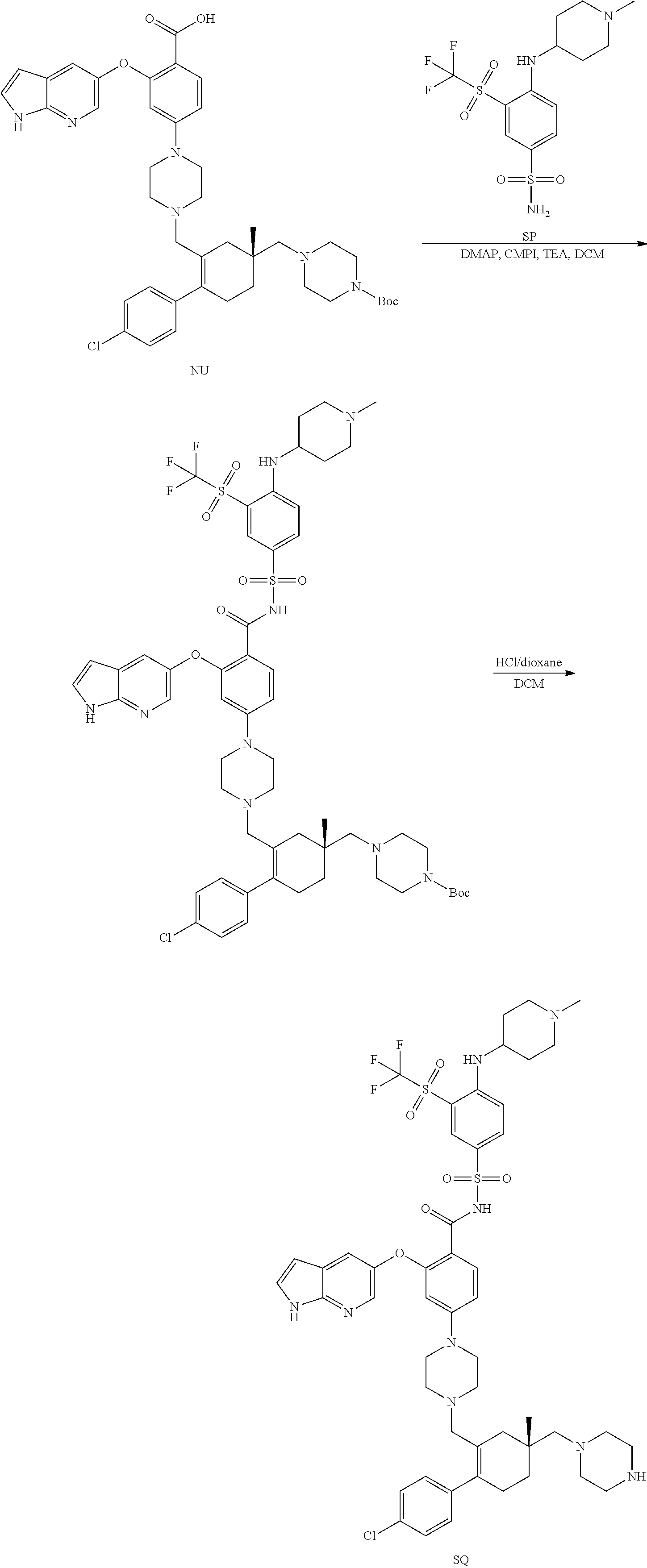

### Step 1—Tert-butyl (R)-4-((6-((4-(3-((1H-pyrrolo[2,3-b]pyridin-5-yl)oxy)-4-(((4-((1-methylpiperidin-4-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate

A solution of (R)-2-((1H-pyrrolo[2,3-b]pyridin-5-yl)oxy)-4-(4-((4-((4-(tert-butoxycarbonyl)piperazin-1-yl)methyl)-4′-chloro-4-methyl-3,4,5,6-tetrahydro-[1,1′-biphenyl]-2-yl)methyl)piperazin-1-yl)benzoic acid (100 mg, 132 μmol, Intermediate NU), 4-((1-methylpiperidin-4-yl)amino)-3-((trifluoromethyl)sulfonyl)benzenesulfonamide (69.1 mg, 172 μmol, Intermediate SP), DMAP (16.2 mg, 132 μmol), CMPI (50.7 mg, 199 μmol), and TEA (20.1 mg, 198 μmol, 27.6 uL) in DCM (1 mL) was stirred at 25° C. for 2 hrs. On completion, the mixture was concentrated in vacuo to give the residue. The crude residue was purified by reversed-phase HPLC (0.8 g/L ammonium bicarbonate) to give the title compound (100 mg) as a white solid. LC-MS (ESI⁺) m/z 1138.0 (M+H)⁺.

### Step 2—(R)-2-((1H-pyrrolo[2,3-b]pyridin-5-yl)oxy)-4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-((1-methylpiperidin-4-yl)amino)-3-((trifluoromethyl)sulfonyl)phenyl)sulfonyl)benzamide

To a solution of tert-butyl (R)-4-((6-((4-(3-((1H-pyrrolo[2,3-b]pyridin-5-yl)oxy)-4-(((4-((1-methylpiperidin-4-yl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (100 mg, 87.8 μmol) in DCM (2 mL) was added HCl/dioxane (4 M, 1 mL). The mixture was then stirred at 25° C. for 0.5 hr. On completion, the mixture was concentrated in vacuo to give the title compound (100 mg, HCl) as a yellow solid. LC-MS (ESI⁺) m/z 1038.3 (M+H)⁺.

### Synthesis of 8-[[(1S)-1-[(2S,4R)-2-[[(1R)-1-(4-ethynylphenyl)-2-hydroxy-ethyl]carbamoyl]-4-hydroxy-pyrrolidine-1-carbonyl]-2,2-dimethyl-propyl]amino]-8-oxo-octanoic Acid (Intermediate SR)

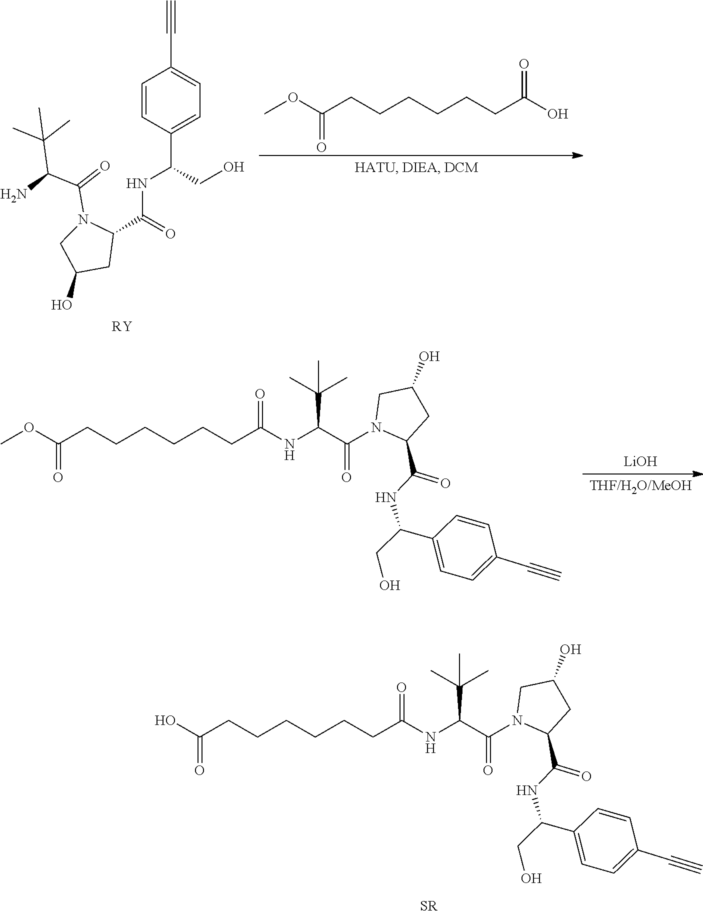

### Step 1—Methyl 8-(((S)-1-((2S,4R)-2-(((R)-1-(4-ethynylphenyl)-2-hydroxyethyl)carbamoyl)-4-hydroxypyrrolidin-1-yl)-3,3-dimethyl-1-oxobutan-2-yl)amino)-8-oxooctanoate

To a solution of 8-methoxy-8-oxooctanoic acid (2.22 g, 11.8 mmol, CAS #3946-32-5) in DCM (100 mL) was added HATU (5.38 g, 14.1 mmol) and DIEA (7.62 g, 59 mmol), and the reaction was stirred for 5 min. Then (2S,4R)-1-((S)-2-amino-3,3-dimethylbutanoyl)-N—((R)-1-(4-ethynylphenyl)-2-hydroxyethyl)-4-hydroxypyrrolidine-2-carboxamide (5 g, 11.8 mmol, Intermediate RY) was added and the mixture was stirred at 20° C. for 0.5 hrs. On completion, the mixture was quenched by addition of H₂O (100 mL) and extracted with DCM (100 mL×3). The organic phase was dried over anhydrous Na₂SO₄, then filtered and the filtrate was concentrated under reduced pressure to give the title compound (10 g) as a colorless oil. LC-MS (ESI⁺) m/z 558.5 (M+H)⁺.

### Step 2—8-(((S)-1-((2S,4R)-2-(((R)-1-(4-ethynylphenyl)-2-hydroxyethyl)carbamoyl)-4-hydroxypyrrolidin-1-yl)-3,3-dimethyl-1-oxobutan-2-yl)amino)-8-oxooctanoic Acid

To a solution of methyl 8-[[(1S)-1-[(2S,4R)-2-[[(1R)-1-(4-ethynylphenyl)-2-hydroxy-ethyl]carbamoyl]-4-hydroxy-pyrrolidine-1-carbonyl]-2,2-dimethyl-propyl]amino]-8-oxo-octanoate (10 g, 17.9 mmol) in H₂O (20 mL), THF (50 mL) and MeOH (50 mL) was added LiOH·H₂O (2.26 g, 53.8 mmol). The mixture was then stirred at 20° C. for 2 hrs. On completion, the mixture was concentrated under reduced pressure to remove organic phase, the residue was diluted with H₂O (200 mL), adjusted to pH 3˜4 by 1M HCl and extracted with DCM (150 mL×3). The combined organic layers were concentrated and purified by RPLC (0.1% HCl condition) to give the title compound (4.6 g, 42% yield) as a white solid. LC-MS (ESI⁺) m/z 544.5 (M+H)⁺.

### Synthesis of (R)-2-((1H-pyrrolo[2,3-b]pyridin-5-yl)oxy)-4-(4-((4-((4-(tert-butoxycarbonyl)-1,4-diazepan-1-yl)methyl)-4′-chloro-4-methyl-3,4,5,6-tetrahydro-[1,1′-biphenyl]-2-yl)methyl)piperazin-1-yl)benzoic Acid (Intermediate SS)

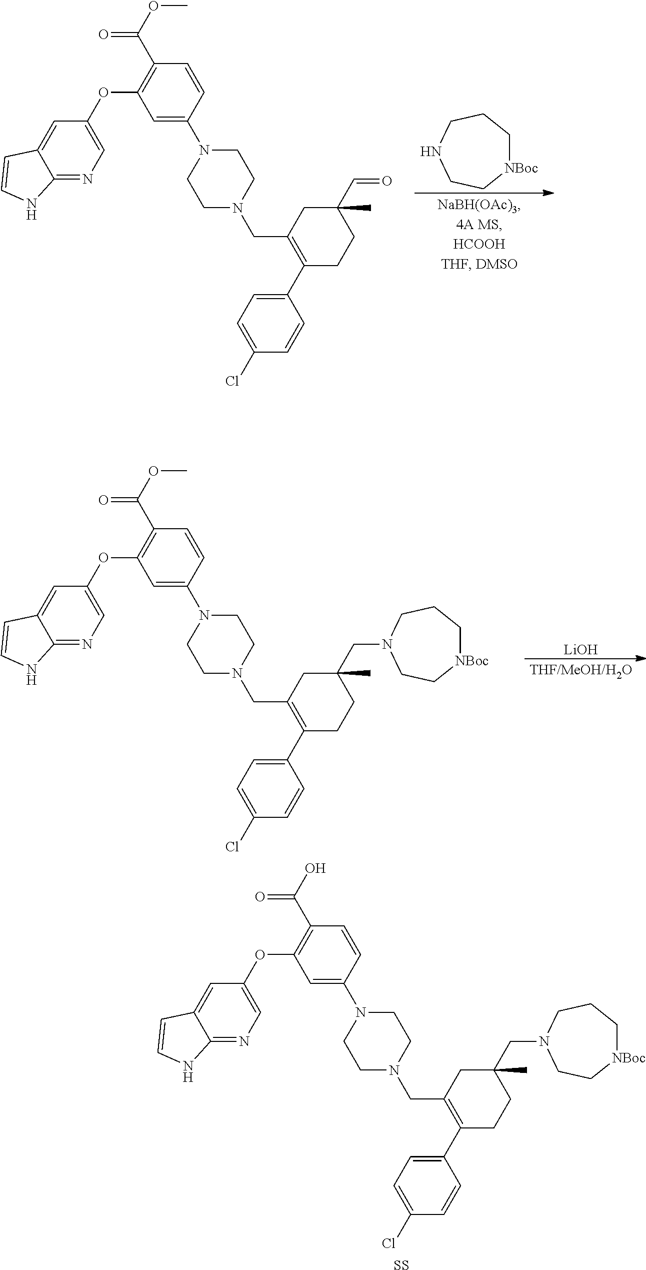

### Step 1—Tert-butyl (R)-4-((6-((4-(3-((1H-pyrrolo[2,3-b]pyridin-5-yl)oxy)-4-(methoxycarbonyl)phenyl)piperazin-1-yl)methyl)-4′-chloro-4-methyl-2,3,4,5-tetrahydro-[1,1′-biphenyl]-4-yl)methyl)-1,4-diazepane-1-carboxylate

To a solution of methyl 4-[4-[[(5R)-2-(4-chlorophenyl)-5-formyl-5-methyl-cyclohexen-1-yl]methyl]piperazin-1-yl]-2-(1H-pyrrolo[2,3-b]pyridin-5-yloxy)benzoate (500 mg, 775 μmol, FA, synthesized via Steps 1-2 of Intermediate NU) in THF (10 mL) and DMSO (5 mL) was added tert-butyl 1,4-diazepane-1-carboxylate (186 mg, 930 μmol, CAS #112275-50-0), HCOOH (112 mg, 2.33 mmol) and 4 Å molecular sieves (1.00 g). The mixture was then stirred at 25° C. for 46 hrs. Then NaBH(OAc)₃ (411 mg, 1.94 mmol) was added and the mixture was stirred at 25° C. for 2 hrs. On completion, the mixture was filtered and concentrated to give a residue. The residue was purified by column chromatography (SiO₂, Petroleum ether:Ethyl acetate=1:1 to DCM:MeOH=30:1) to give the title compound (607 mg, 58% yield) as a white solid. LC-MS (ESI⁺) m/z 783.4 (M+H)⁺.

### Step 2—(R)-2-((1H-pyrrolo[2,3-b]pyridin-5-yl)oxy)-4-(4-((4-((4-(tert-butoxycarbonyl)-1,4-diazepan-1-yl)methyl)-4′-chloro-4-methyl-3,4,5,6-tetrahydro-[1,1′-biphenyl]-2-yl)methyl)piperazin-1-yl)benzoic Acid

To a solution of tert-butyl 4-[[(1R)-4-(4-chlorophenyl)-3-[[4-[4-methoxycarbonyl-3-(1H-pyrrolo[2,3-b]pyridin-5-yloxy)phenyl]piperazin-1-yl]methyl]-1-methyl-cyclohex-3-en-1-yl]methyl]-1,4-diazepane-1-carboxylate (450 mg, 574 μmol) in THF (8 mL), MeOH (8 mL) and H₂O (4 mL) was added LiOH·H₂O (121 mg, 2.87 mmol). The mixture was stirred at 50° C. for 12 hrs. The mixture was adjusted to pH of 6 by adding 1M HCl solution, diluted with H₂O (10 mL) and extracted with DCM/THF (3:1, 30 mL×3). The combined organic layer was dried over anhydrous Na₂SO₄, filtered and the filtrate was concentrated to give the title compound (400 mg) as a white solid. LC-MS (ESI⁺) m/z 769.3 (M+H)⁺.

### Synthesis of (R)-4-(4-((4-((1,4-diazepan-1-yl)methyl)-4′-chloro-4-methyl-3,4,5,6-tetrahydro-[1,1′-biphenyl]-2-yl)methyl)piperazin-1-yl)-2-((1H-pyrrolo[2,3-b]pyridin-5-yl)oxy)-N-((4-((3-morpholinopropyl)amino)-3-((trifluoromethyl)sulfonyl)phenyl)sulfonyl)benzamide (Intermediate ST)

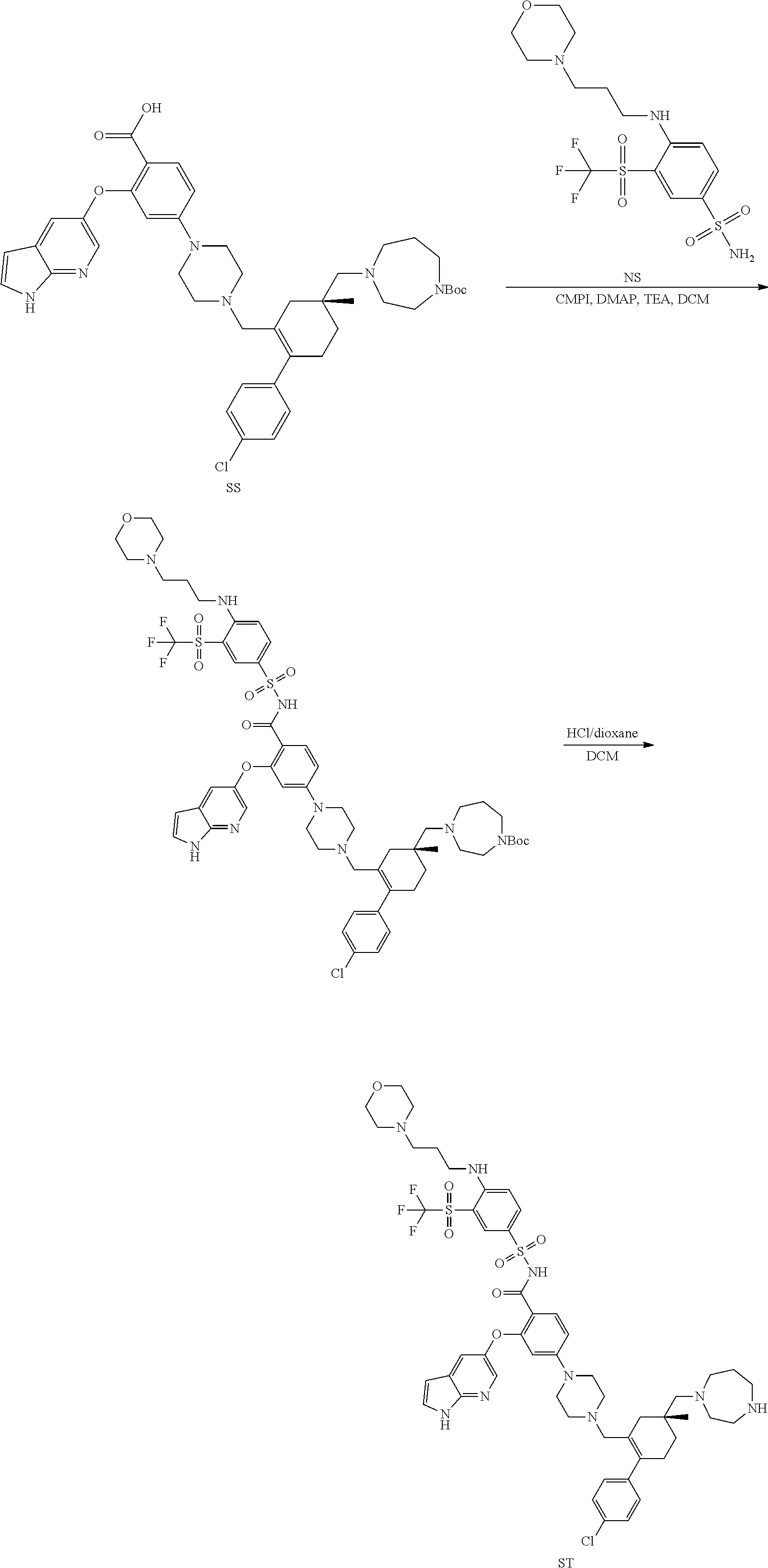

### Step 1—Tert-butyl (R)-4-((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-1,4-diazepane-1-carboxylate

To a solution of 4-[4-[[(5R)-5-[(4-tert-butoxycarbonyl-1,4-diazepan-1-yl)methyl]-2-(4-chlorophenyl)-5-methylcyclohexen-1-yl]methyl]piperazin-1-yl]-2-(1H-pyrrolo[2,3-b]pyridin-5-yloxy)benzoic acid (400 mg, 519 μmol, Intermediate SS) in DCM (10 mL) was added DMAP (63.5 mg, 520 μmol), TEA (158 mg, 1.56 mmol, 217 uL) and CMPI (199 mg, 780 μmol). Then 4-(3-morpholinopropylamino)-3-(trifluoromethylsulfonyl)benzenesulfonamide (224 mg, 520 μmol, Intermediate NS) was added and the mixture was then stirred at 25° C. for 2 hrs. On completion, the mixture was concentrated to give the crude product. The crude product was purified by reversed-phase HPLC (0.1% FA condition) to give the title compound (260 mg, 41% yield, FA) as a white solid. LC-MS (ESI⁺) m/z 1182.4 (M+H)⁺.

### Step 2—(R)-4-(4-((4-((1,4-diazepan-1-yl)methyl)-4′-chloro-4-methyl-3,4,5,6-tetrahydro-[1,1′-biphenyl]-2-yl)methyl)piperazin-1-yl)-2-((1H-pyrrolo[2,3-b]pyridin-5-yl)oxy)-N-((4-((3-morpholinopropyl)amino)-3-((trifluoromethyl)sulfonyl)phenyl)sulfonyl)benzamide

To a solution of tert-butyl (R)-4-((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-1,4-diazepane-1-carboxylate (249 mg, 210 μmol, FA) in DCM (12 mL) was added HCl/dioxane (4 M, 3 mL). The mixture was then stirred at 25° C. for 2 hrs. On completion, the reaction mixture was concentrated in vacuo to remove solvent to give the title compound (229 mg) as a light yellow solid. LC-MS (ESI⁺) m/z 1082.3 (M+H)⁺.

### Synthesis of 2-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)acetic acid (Intermediate SU)

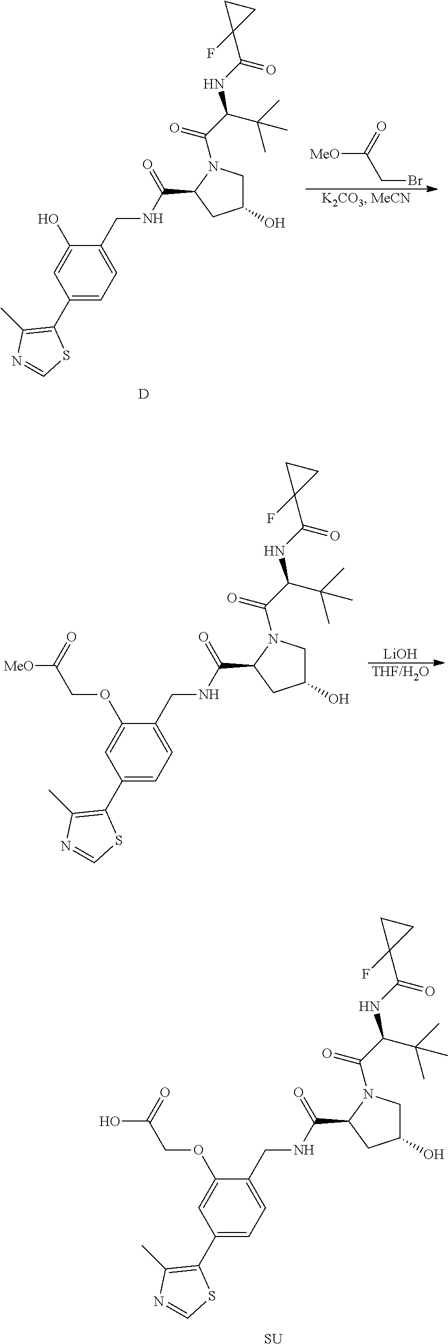

### Step 1—Methyl 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetate

To a solution of (2S,4R)-1-((S)-2-(1-fluorocyclopropane-1-carboxamido)-3,3-dimethylbutanoyl)-4-hydroxy-N-(2-hydroxy-4-(4-methylthiazol-5-yl)benzyl)pyrrolidine-2-carboxamide (5.48 g, 10.29 mmol, Intermediate D) in MeCN (60 mL) was added potassium carbonate (3.41 g, 24.7 mmol) and then methyl 2-bromoacetate (1.32 mL, 13.89 mmol). After stirring for 20 hrs, the volatiles were removed in vacuo and the residue was partitioned between EtOAc and H₂O, washed with brine, dried over MgSO₄, filtered, and concentrated. The residue was purified by SiO₂ chromatography (0-15% MeOH/DCM) to yield the title compound (4.80 g). LC-MS (ESI⁺) m/z 605.9 (M+H)⁺.

### Step 2—2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl)phenoxy)acetic Acid

To a solution of methyl 2-(2-(((2S,4R)-1-((S)-2-(1-fluorocyclopropane-1-carboxamido)-3,3-dimethylbutanoyl)-4-hydroxypyrrolidine-2-carboxamido)methyl)-5-(4-methylthiazol-5-yl) phenoxy)acetate (4.80 g, 7.94 mmol) in THF/MeOH (2:1, 60 mL) was added 1N LiOH (8.73 mL, 8.73 mmol). After stirring for 20 hrs, the solution was neutralized by addition of 1N HCl and the volatiles were removed in vacuo. Then, DMSO (20 mL) was added and the solution was purified by RP-HPLC (Teledyne Method) (10-100% MeCN/H₂O with 0.1% TFA modifier). After lyophilization, the title compound (1.9 g, 40% yield) was obtained as pale yellow solid. LC-MS (ESI+) m/z 591.5 (M+H)⁺.

### Synthesis of 2-((1R,4r)-4-(4-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carbonyl)cyclohexyl)acetic Acid (Intermediate SV)

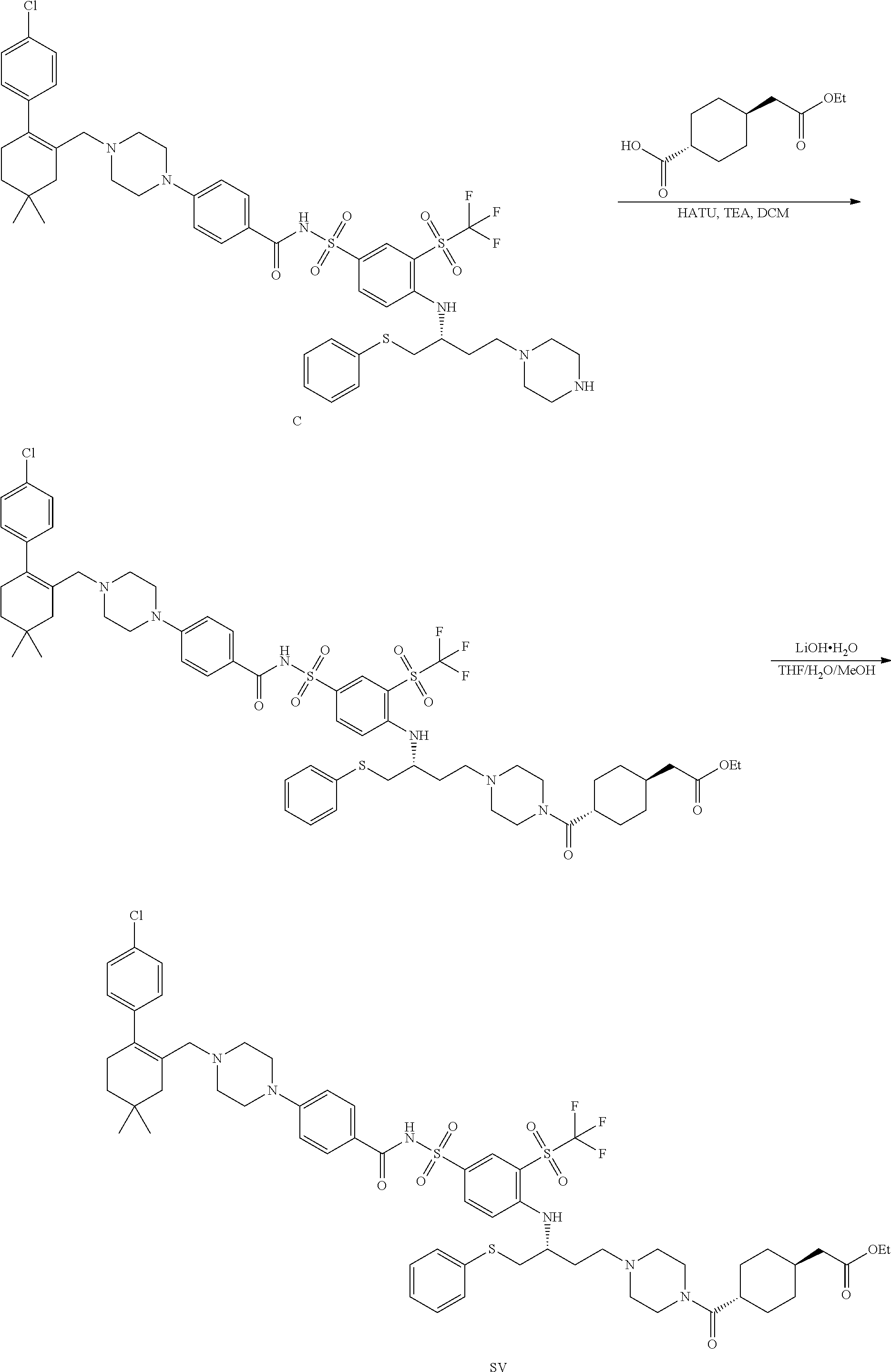

### Step 1—Ethyl 2-((1R,4r)-4-(4-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carbonyl)cyclohexyl)acetate

To a solution of (R)-4-(4-((4′-chloro-4,4-dimethyl-3,4,5,6-tetrahydro-[1,1′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide (100 mg, 102 μmol, Intermediate C) and TEA (51.9 mg, 513 μmol) in DCM (3 mL) was added (1r,4r)-4-(2-ethoxy-2-oxoethyl)cyclohexanecarboxylic acid (22.0 mg, 102 μmol, CAS #15177-66-9) and HATU (41.0 mg, 107 μmol). The mixture was then stirred at 25° C. for 0.5 h under N₂ atmosphere. On completion, the reaction mixture was filtered and concentrated under reduced pressure to give the title compound (230 mg) as brown gum. LC-MS (ESI+) m/z 585.9 (M+H)⁺/2.

### Step 2—2-((1R,4r)-4-(4-(R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carbonyl)cyclohexyl)acetic Acid

A mixture of ethyl 2-((1R,4r)-4-(4-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carbonyl)cyclohexyl)acetate (230 mg, 196 μmol) and LiOH·H₂O (41.2 mg, 983 μmol) in H₂O (0.5 mL), THF (1.5 mL) and MeOH (0.5 mL) was degassed, and then the mixture was stirred at 25° C. for 1 h. On completion, filtered and concentrated to dryness under reduced pressure at 45° C. to give the title compound (70 mg) as white solid. LC-MS (ESI+) m/z 571.7 (M+H)⁺/2.

### Synthesis of 2-((1S,4r)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)cyclohexyl)acetic Acid (Intermediate SW)

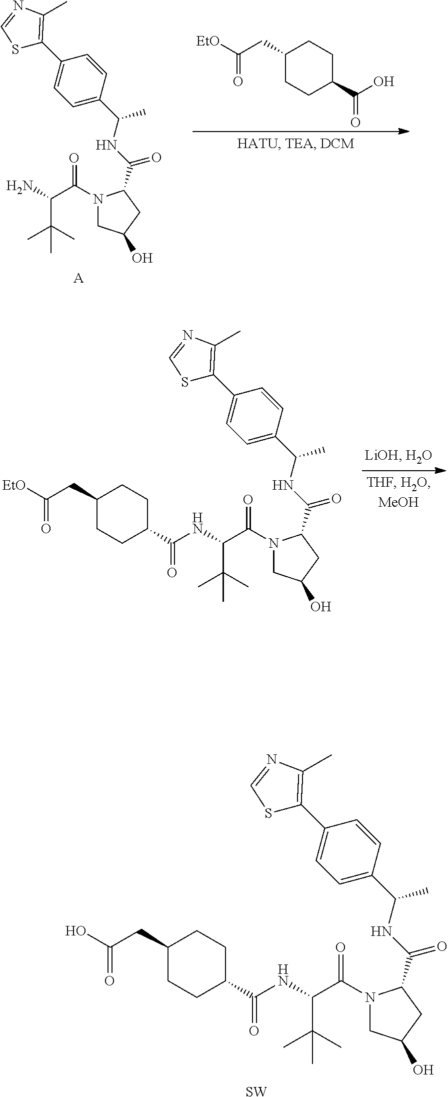

### Step 1—Ethyl 2-((1S,4r)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)cyclohexyl)acetate

To a solution of (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide (200 mg, 449 μmol, Intermediate A) and (1r,4r)-4-(2-ethoxy-2-oxoethyl)cyclohexane-1-carboxylic acid (96.4 mg, 449 μmol, CAS #880104-40-5) in DCM (8 mL) was added HATU (179 mg, 472 μmol) and TEA (227 mg, 2.25 mmol, 313 uL). The mixture was stirred at 25° C. for 12 h. On completion, the reaction mixture was poured into ice water (20 mL) and extracted with DCM (20 mL×3). The combined organic layers were washed with brine (200 mL×2), dried over sodium sulfate, filtered and concentrated under reduced pressure to give a residue. The residue was purified by column chromatography (SiO₂, Petroleum ether/Ethyl acetate=1/0 to 0/1) to give the title compound (400 mg, 93% yield) as a yellow solid. ¹H NMR (400 MHz, DMSO-d₆) δ=8.98 (s, 1H), 8.38 (d, J=7.8 Hz, 1H), 7.66 (br d, J=9.0 Hz, 1H), 7.45-7.36 (m, 4H), 5.11 (d, J=3.6 Hz, 1H), 4.96-4.87 (m, 1H), 4.48 (d, J=9.4 Hz, 1H), 4.41 (t, J=8.0 Hz, 1H), 4.27 (br s, 1H), 3.63-3.54 (m, 2H), 3.09 (q, J=7.4 Hz, 6H), 2.69 (s, 2H), 2.45 (s, 3H), 2.34-2.25 (m, 1H), 2.16 (br d, J=6.8 Hz, 3H), 1.81-1.61 (m, 7H), 1.37 (br d, J=7.0 Hz, 3H), 0.97-0.90 (m, 12H).

### Step 2—2-((1S,4r)-4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)cyclohexyl)acetic Acid

To a solution of ethyl 2-[4-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamoyl]cyclohexyl acetate (350 mg, 546 μmol) in THF (4 mL). H₂O (4 mL), and MeOH (4 mL) was added LiOH·H₂O (115 mg, 2.73 mmol). The mixture was stirred at 25° C. for 2 h. On completion, the reaction was poured into ice water (12 mL) and extracted with DCM (20 mL×3). The aqueous phase was acidified with 1N HCl to pH=4 and extracted with DCM (20 mL×3), The combined organic phase is washed with brine (20 mL×3), dried over sodium sulfate, filtered and concentrated under reduced pressure to give the title compound (230 mg) as a yellow solid. ¹H NMR (400 MHz, DMSO-d₆) δ 12.01-11.94 (m, 1H), 8.98 (s, 1H), 8.36 (d, J=7.8 Hz, 1H), 7.64 (d, J=9.2 Hz, 1H), 7.45-7.41 (m, 2H), 7.39-7.36 (m, 2H), 5.09 (br s, 1H), 4.91 (quin, J=7.2 Hz, 1H), 4.48 (d, J=9.4 Hz, 1H), 4.42 (t, J=8.0 Hz, 1H), 4.28 (br s, 1H), 4.03 (q, J=7.2 Hz, 1H), 3.62-3.56 (m, 2H), 2.45 (s, 3H), 2.34-2.25 (m, 1H), 2.09 (d, J=6.8 Hz, 2H), 1.99 (s, 2H), 1.81-1.69 (m, 5H), 1.37 (d, J=7.0 Hz, 3H), 1.17 (t, J=7.2 Hz, 2H), 0.95-0.91 (m, 10H).

### Synthesis of (R)-4-(4-((4′-chloro-4,4-dimethyl-3,4,5,6-tetrahydro-[1,1′-biphenyl]-2-yl)methyl)piperazin-1-yl)-N-((4-((4-(methylamino)-1-(phenylthio)butan-2-yl)amino)-3-((trifluoromethyl)sulfonyl)phenyl)sulfonyl)benzamide (Intermediate SX)

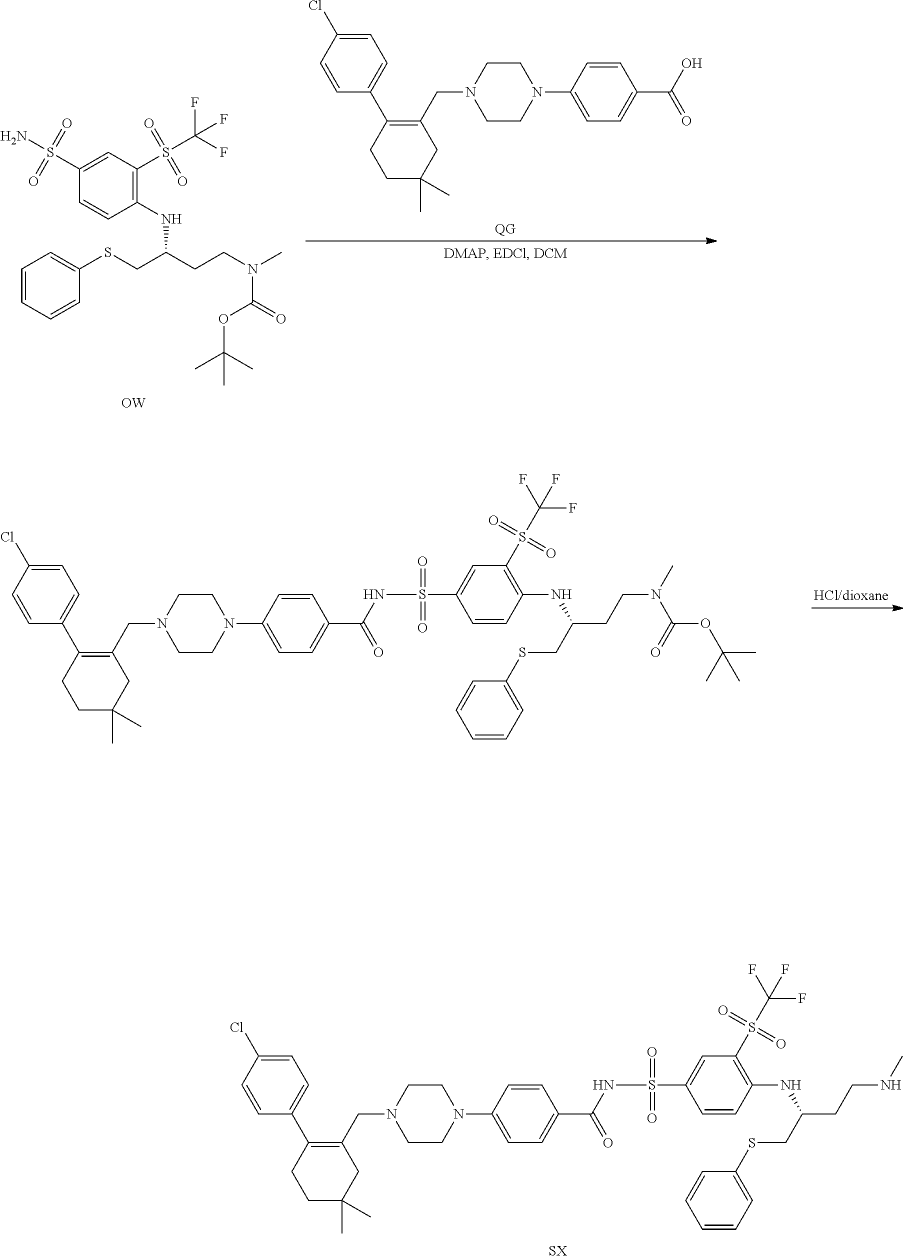 [SMILES: CN(CC[C@H](CSc1ccccc1)Nc1ccc(S(N)(=O)=O)cc1S(=O)(=O)C(F)(F)F)C(=O)OC(C)(C)C ; CC1(C)CCC(c2ccc(Cl)cc2)=C(CN2CCN(c3ccc(C(=O)O)cc3)CC2)C1 ; "OW" ; CN(CC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F)C(=O)OC(C)(C)C ; CNCC[C@H](CSc1ccccc1)Nc1ccc(S(=O)(=O)NC(=O)c2ccc(N3CCN(CC4=C(c5ccc(Cl)cc5)CCC(C)(C)C4)CC3)cc2)cc1S(=O)(=O)C(F)(F)F]

### Step 1—(R)-tert-butyl (3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)(methyl)carbamate

A mixture of 4-(4-((4′-chloro-4,4-dimethyl-3,4,5,6-tetrahydro-[1,1′-biphenyl]-2-yl)methyl)piperazin-1-yl)benzoic acid (169 mg, 385 μmol, Intermediate QG), (R)-tert-butyl methyl (4-(phenylthio)-3-((4-sulfamoyl-2-((trifluoromethyl)sulfonyl)phenyl)amino)butyl)carbamate (230 mg, 308 μmol, Intermediate OW), DMAP (188 mg, 1.54 mmol), EDCI (295 mg, 1.54 mmol) in DCM (4.6 mL) was degassed and purged with N₂ three times. Then the mixture was stirred at 25° C. for 12 h under N₂ atmosphere. On completion, the reaction mixture was quenched with H₂O (5 mL) and then extracted with DCM (5 mL×3). The combined organic layers were washed with brine (2 mL×3), dried over Na₂SO₄, filtered and concentrated under reduced pressure to give a residue. The residue was purified by prep-HPLC (column: Phenomenex Luna C18 100*30 mm*5 um; mobile phase: [water (FA)-ACN]; B %: 52%-82%, 8 min) to give the title compound (190 mg, 58% yield) as a white solid. ¹H NMR (400 MHz, DMSO-d₆) δ=8.14 (s, 1H), 7.99-7.86 (m, 1H), 7.73 (br d, J=8.8 Hz, 2H), 7.36 (br dd, J=7.6, 19.2 Hz, 4H), 7.26 (br t, J=7.6 Hz, 2H), 7.19 (br d, J=7.6 Hz, 1H), 7.13 (br d, J=8.0 Hz, 2H), 6.89 (br d, J=8.4 Hz, 4H), 3.86 (br d, J=7.2 Hz, 1H), 3.31-3.26 (m, 4H), 3.16-3.10 (m, 1H), 2.92-2.76 (m, 2H), 2.72 (s, 3H), 2.55 (s, 1H), 2.43-2.32 (m, 2H), 2.24 (br s, 2H), 2.00 (br s, 3H), 1.81 (br s, 1H), 1.45 (br t, J=6.0 Hz, 2H), 1.33-1.12 (m, 11H), 1.08 (s, 1H), 0.98 (s, 6H).

### Step 2—(R)-4-(4-((4′-chloro-4,4-dimethyl-3,4,5,6-tetrahydro-[1,1′-biphenyl]-2-yl)methyl)piperazin-1-yl)-N-((4-((4-(methylamino)-1-(phenylthio)butan-2-yl)amino)-3-((trifluoromethyl)sulfonyl)phenyl)sulfonyl)benzamide

A mixture of (R)-tert-butyl (3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)(methyl)carbamate (190 mg, 187 μmol) in HCl/dioxane (2 mL) was degassed and purged with N₂ three times, and then the mixture was stirred at 25° C. for 1 h under N₂ atmosphere. On completion, the reaction mixture was filtered and concentrated under reduced pressure to give the title compound (220 mg, HCl) as a white solid. LC-MS (ESI+) m/z 459.6 (M/2)⁺.

### Synthesis of (2S,4R)-4-hydroxy-1-(3-methyl-2-(3-methylisoxazol-5-yl)butanoyl)pyrrolidine-2-carboxylic Acid (Intermediate SY)

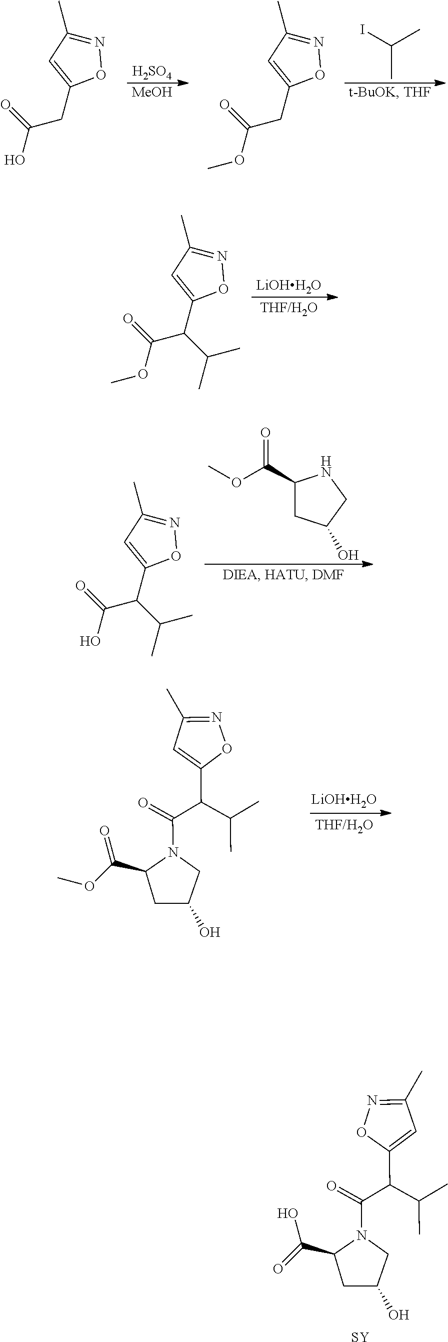

### Step 1—Methyl 2-(3-methylisoxazol-5-yl)acetate

A mixture of 2-(3-methylisoxazol-5-yl)acetic acid (7.00 g, 49.6 mmol) and H₂SO₄ (243 mg. 2.48 mmol, 132 uL) in MeOH (70 mL) was stirred at 70° C. for 2 h under N₂ atmosphere. On completion, the reaction mixture was quenched by addition with NaHCO₃ (70 mL), and extracted with EtOAc (30 mL×9). The organic layers were washed with brine (40 mL×3), dried over Na₂SO₄, filtered and concentrated under reduced pressure to give a residue. The product was purified by column chromatography (SiO₂, Petroleum ether/Ethyl acetate=10/1 to 1/1) to give the title compound (7.00 g, 89% yield) as yellow oil. ¹H NMR (400 MHz, DMSO-d₆) δ=6.27 (s, 1H), 3.96 (s, 2H), 3.66 (s, 3H), 2.21 (s, 3H).

### Step 2—Methyl 3-methyl-2-(3-methylisoxazol-5-yl)butanoate

A mixture of methyl 2-(3-methylisoxazol-5-yl)acetate (7.96 g, 51.3 mmol), and t-BuOK (1 M, 102 mL) in THF (100 mL) was added in 2-iodopropane (12.2 g, 71.8 mmol) in 0° C., and then the mixture was stirred at 0-25° C. for 16 h. On completion, the reaction mixture was quenched by addition of ice water (10 mL), and extracted with EtOAc (10 mL×3). The organic layers were washed with brine (10 mL×3), dried over Na₂SO₄, filtered and concentrated to dryness under reduced pressure at 45° C. This product was purified by column chromatography (SiO₂, Petroleum ether/Ethyl acetate=20/1 to 0/1) to give the title compound (4.20 g, 37% yield) as yellow gum. ¹H NMR (400 MHz, DMSO-d₆) δ=6.30 (s, 1H), 3.75 (d, J=8.4 Hz, 1H), 3.65 (s, 3H), 2.30 (td, J=6.8, 8.5 Hz, 1H), 2.21 (s, 3H), 0.93 (d, J=6.4 Hz, 3H), 0.82 (d, J=6.8 Hz, 3H).

### Step 3—3-Methyl-2-(3-methylisoxazol-5-yl)butanoic Acid

A mixture of methyl 3-methyl-2-(3-methylisoxazol-5-yl)butanoate (3.02 g, 15.3 mmol) and LiOH·H₂O (2.57 g, 61.3 mmol) in THF (25 mL) and H₂O (5 mL), and then the mixture was stirred at 25° C. for 8 h under N₂ atmosphere. On completion, filtered and concentrated to dryness under reduced pressure at 45° C. to give the title compound (2.9 g) as yellow solid. LC-MS (ESI+) m/z 184.0 (M+H)⁺.

### Step 4—(2S,4R)-methyl 4-hydroxy-1-(3-methyl-2-(3-methylisoxazol-5-yl)butanoyl)pyrrolidine-2-carboxylate

To a solution of 3-methyl-2-(3-methylisoxazol-5-yl)butanoic acid (2.90 g, 13.9 mmol) and DIEA (9.71 mL) in DMF (30 mL) was added (2S,4R)-methyl 4-hydroxypyrrolidine-2-carboxylate (2.53 g, 13.9 mmol, HCl salt) and HATU (5.83 g, 15.3 mmol), and the mixture was stirred at 25° C. for 12 h. On completion, the reaction mixture was quenched by addition with H₂O (50 mL) at 20° C., and extracted with EtOAc (25 mL×3). The organic layers were washed with brine (25 mL×3), dried over Na₂SO₄, filtered and concentrated under reduced pressure to give a residue. The product was purified by column chromatography (SiO₂, Petroleum ether/Ethyl acetate=3/1 to 0/1) to give the title compound (2.90 g, 67% yield) as colorless oil. ¹H NMR (400 MHz, DMSO-d₆) δ=6.22 (s, 1H), 6.17 (s, 1H), 4.30-4.24 (m, 2H), 4.03 (q, J=7.2 Hz, 1H), 3.84 (d, J=9.2 Hz, 1H), 3.80-3.75 (m, 2H), 3.62 (s, 3H), 3.55 (s, 3H), 3.48-3.42 (m, 2H), 2.25 (td, J=6.8, 9.2 Hz, 2H), 2.21 (s, 3H), 2.19 (s, 3H), 2.13-2.06 (m, 2H), 1.95-1.85 (m, 2H), 0.95 (t, J=6.8 Hz, 6H), 0.78 (d, J=6.4 Hz, 6H).

### Step 5—(2S,4R)-4-hydroxy-1-(3-methyl-2-(3-methylisoxazol-5-yl)butanoyl)pyrrolidine-2-carboxylic Acid

A mixture of (2S,4R)-methyl 4-hydroxy-1-(3-methyl-2-(3-methylisoxazol-5-yl)butanoyl)pyrrolidine-2-carboxylate (2.90 g, 9.34 mmol) and LiOH·H₂O (1.57 g, 37.4 mmol) in H₂O (6 mL) and THF (24 mL), and then the mixture was stirred at 25° C. for 1 h. On completion, filtered and concentrated to dryness under reduced pressure at 45° C. to give the title compound (2.6 g) as yellow solid. LC-MS (ESI+) m/z 297.1 (M+H)⁺.

### Synthesis of 2-(2-(((2S,4R)-4-hydroxy-1-(3-methyl-2-(3-methylisoxazol-5-yl)butanoyl)pyrrolidine-2-carboxamido)methyl)-5-(4-methylthiazol-5-yl)phenoxy)acetic Acid (Intermediate SZ)

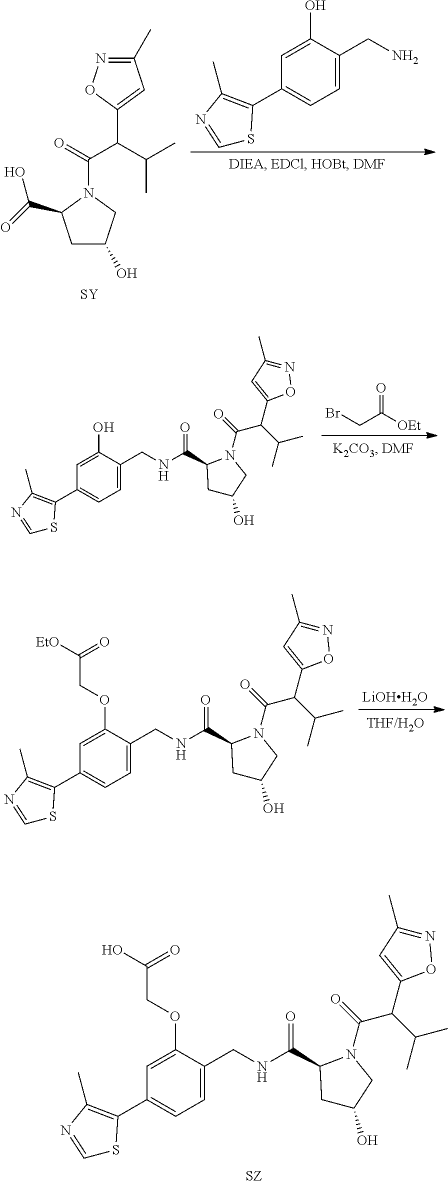

### Step 1—(2S,4R)-4-hydroxy-N-(2-hydroxy-4-(4-methylthiazol-5-yl)benzyl)-1-(3-methyl-2-(3-methylisoxazol-5-yl)butanoyl)pyrrolidine-2-carboxamide

A mixture of (2S,4R)-4-hydroxy-1-(3-methyl-2-(3-methylisoxazol-5-yl)butanoyl)pyrrolidine-2-carboxylic acid (2.50 g, 8.44 mmol, Intermediate SY), 2-(aminomethyl)-5-(4-methylthiazol-5-yl)phenol (1.86 g, 8.44 mmol, CAS #1448190-11-1), DIEA (7.35 mL). EDCI (2.43 g, 12.7 mmol) and HOBt (1.71 g, 12.7 mmol) in DMF (25 mL), and then the mixture was stirred at 25° C. for 6 h under N₂ atmosphere. On completion, the reaction mixture was quenched with H₂O (30 mL) at 20° C., and extracted with EtOAc (20 mL×3). The organic layers were washed with brine (20 mL×3), dried over Na₂SO₄, filtered and concentrated under reduced pressure to give a residue. This product was purified by reversed-phase HPLC (0.1% FA condition) to give the title compound (500 mg, 12% yield) as orange solid. ¹H NMR (400 MHz, DMSO-d₆) δ=9.80 (s, 1H), 8.96 (s, 1H), 8.39 (t, J=6.0 Hz, 1H), 7.29-7.23 (m, 1H), 6.93-6.90 (m, 2H), 6.22 (s, 1H), 5.13-5.08 (m, 1H), 4.46 (br t, J=7.6 Hz, 1H), 4.40-4.37 (m, 1H), 4.21 (br d, J=6.4 Hz, 2H), 3.74 (d, J=9.6 Hz, 1H), 3.60-3.50 (m, 2H), 2.45 (s, 3H), 2.26 (br dd, J=2.8, 6.7 Hz, 1H), 2.20 (s, 3H), 2.05-1.98 (m, 2H), 0.96 (s, 3H), 0.78 (s, 3H).

### Step 2—Ethyl 2-(2-(((2S,4R)-4-hydroxy-1-(3-methyl-2-(3-methylisoxazol-5-yl)butanoyl)pyrrolidine-2-carboxamido)methyl)-5-(4-methylthiazol-5-yl)phenoxy)acetate

A mixture of ethyl 2-bromoacetate (226 mg, 1.35 mmol), (2S,4R)-4-hydroxy-N-(2-hydroxy-4-(4-methylthiazol-5-yl)benzyl)-1-(3-methyl-2-(3-methylisoxazol-5-yl)butanoyl)pyrrolidine-2-carboxamide (450 mg, 902 μmol), and K₂CO₃ (125 mg, 902 μmol) in DMF (5 mL), and then the mixture was stirred at 20° C. for 4 h under N₂ atmosphere. On completion, the reaction mixture was quenched by addition of H₂O (5 mL) at 20° C., and extracted with EtOAc (5 mL×3). The organic layers were washed with brine (5 mL×3), dried over Na₂SO₄, filtered and concentrated under reduced pressure to give the title compound (550 mg) as yellow gum. LC-MS (ESI+) m/z 585.2 (M+H)⁺.

### Step 3—2-(2-(((2S,4R)-4-hydroxy-1-(3-methyl-2-(3-methylisoxazol-5-yl)butanoyl)pyrrolidine-2-carboxamido)methyl)-5-(4-methylthiazol-5-yl)phenoxy)acetic Acid

A mixture of ethyl 2-(2-(((2S,4R)-4-hydroxy-1-(3-methyl-2-(3-methylisoxazol-5-yl)butanoyl) pyrrolidine-2-carboxamido)methyl)-5-(4-methylthiazol-5-yl)phenoxy)acetate (550 mg, 940 μmol) and LiOH·H₂O (158 mg, 3.76 mmol) in H₂O (1 mL) and THF (4 mL), and then the mixture was stirred at 25° C. for 1 h. On completion, filtered and concentrated to dryness under reduced pressure at 45° C., to give the title compound (450 mg) as yellow solid. LC-MS (ESI+) m/z 557.5 (M+H)⁺.

### Synthesis of (S)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanoic Acid (Intermediate TA) and (R)-3-((2S,4R)-1-((S)-2-(1-fluorocyclopropane-1-carboxamido)-3,3-dimethylbutanoyl)-4-hydroxypyrrolidine-2-carboxamido)-3-(4-(4-methylthiazol-5-yl)phenyl)propanoic Acid (Intermediate UH)

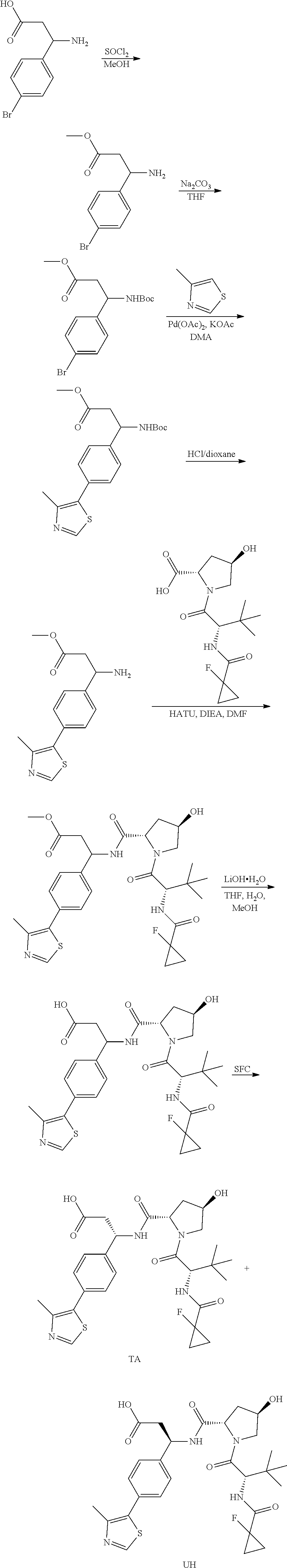

### Step 1—Methyl 3-amino-3-(4-bromophenyl)propanoate

To a solution of methyl 3-amino-3-(4-bromophenyl)propanoic acid (11 g, 45.1 mmol) in MeOH (132 mL) was added SOCl₂ (45.1 g, 379 mmol) at 0° C. The mixture was then stirred at 25° C. for 12 h. On completion, the reaction mixture was poured into ice water (100 mL) and extracted with EtOAc (100 mL×3), and the aqueous phase was basified with NaHCO₃(sat aq) to pH=9. The combined organic layers were washed with brine (200 mL×3), dried over sodium sulfate, filtered and concentrated under reduced pressure to give a residue. The residue was purified by flash silica gel chromatography (ISCO®; 80 g SepaFlash® Silica Flash Column, Eluent of 0˜10% Ethyl acetate/Petroleum ether gradient @ 80 mL/min) to give the title compound (7 g, 60% yield) as a yellow solid. ¹H NMR (400 MHz, CDCl₃) δ 8.73 (br s, 2H), 7.50 (br s, 2H), 7.45 (br s, 2H), 4.73 (brs, 1H), 3.63 (br s, 3H), 3.26 (br d, J=13.2 Hz, 1H), 2.99 (br d, J=13.2 Hz, 1H).

### Step 2—Methyl 3-(4-bromophenyl)-3-((tert-butoxycarbonyl)amino)propanoate

A solution of methyl 3-amino-3-(4-bromophenyl)propanoate (5 g, 19.3 mmol), (Boc)₂O (8.46 g, 38.7 mmol), and Na₂CO₃ (4.11 g, 38.7 mmol) in THF (50 mL) was stirred at 20° C. for 12 h. On completion, the reaction was poured into ice water (50 mL) and extracted with EtOAc (50 mL×3). The combined organic phase is washed with brine (50 mL×3), dried over sodium sulfate, filtered and concentrated under reduced pressure to give a residue. The residue was purified by flash silica gel chromatography (ISCO®; 80 g SepaFlash® Silica Flash Column, Eluent of 0˜10% Ethyl acetate/Petroleum ether gradient @80 mL/min) to give the title compound (5.6 g, 81% yield) as a white solid. LC-MS (ESI+) m/z 380.0 (M+Na)⁺.

### Step 3—Methyl 3-((tert-butoxycarbonyl)amino)-3-(4-(4-methylthiazol-5-yl)phenyl)propanoate

To a solution of methyl 3-(4-bromophenyl)-3-((tert-butoxycarbonyl)amino)propanoate (7.3 g, 20.4 mmol) and 4-methylthiazole (4.04 g, 40.8 mmol, 3.71 mL) in DMA (25 mL) was added KOAc (4.00 g, 40.8 mmol) and Pd(OAc)₂ (229 mg, 1.02 mmol). The mixture was then stirred at 120° C. for 12 h. On completion, the reaction mixture was poured into ice water (25 mL) and extracted with EtOAc (25 mL×3). The combined organic layers were washed with brine (25 mL×3), dried over sodium sulfate, filtered and concentrated under reduced pressure to give a residue. The residue was purified by column chromatography (SiO₂, Petroleum ether/Ethyl acetate=1/0 to 3/1) to give the title compound (3 g, 39% yield) as a yellow oil. ¹H NMR (400 MHz, DMSO-d₆) δ 8.99 (s, 1H), 7.55 (br d, J=8.8 Hz, 1H), 7.47-7.43 (m, 2H), 7.42-7.38 (m, 2H), 5.02-4.91 (m, 1H), 3.57 (s, 3H), 2.76-2.70 (m, 2H), 2.45 (s, 3H), 1.36 (s, 9H).

### Step 4—Methyl 3-amino-3-(4-(4-methylthiazol-5-yl)phenyl)propanoate

To a solution of methyl 3-((tert-butoxycarbonyl)amino)-3-(4-(4-methylthiazol-5-yl)phenyl)propanoate (2 g, 5.31 mmol) in HCl/dioxane (4 M, 22.22 mL) was stirred at 25° C. for 12 h. On completion, the reaction mixture was concentrated under reduced pressure to give the title compound (2.1 g) as a yellow solid. LC-MS (ESI+) m/z 277.1 (M+H)⁺.

### Step 5—Methyl 3-((2S,4R)-1-((S)-2-(1-fluorocyclopropanecarboxamido)-3,3-dimethylbutanoyl)-4-hydroxypyrrolidine-2-carboxamido)-3-(4-(4-methylthiazol-5-yl)phenyl)propanoate

To a solution of (2S,4R)-1-((S)-2-(1-fluorocyclopropanecarboxamido)-3,3-dimethylbutanoyl)-4-hydroxypyrrolidine-2-carboxylic acid (2.66 g, 8.06 mmol) and HATU (2.14 g, 5.64 mmol) in DMF (20 mL) was stirred at 25° C. for 30 min. Then methyl 3-amino-3-(4-(4-methylthiazol-5-yl)phenyl)propanoate (2.1 g, 5.37 mmol, HCl) and DIEA (3.47 g, 26.9 mmol, 4.68 mL) was added and the mixture was stirred at 25° C. for 2 h. On completion, the reaction mixture was poured into ice water (40 mL) and extracted with EtOAc (80 mL×3). The combined organic layers were washed with brine (80 mL×3), dried over sodium sulfate, filtered and concentrated under reduced pressure to give a residue. The residue was purified by flash silica gel chromatography (ISCO®; 20 g ScpaFlash® Silica Flash Column, Eluent of 0˜100% Ethyl acetate/Petroleum ether gradient @80 mL/min) to give the title compound (1.8 g, 52% yield) as a yellow solid. LC-MS (ESI⁺) m/z 589.7 (M+H)⁺.

### Step 6—3-((2S,4R)-1-((S)-2-(1-fluorocyclopropanecarboxamido)-3,3-dimethylbutanoyl)-4-hydroxypyrrolidine-2-carboxamido)-3-(4-(4-methylthiazol-5-yl)phenyl)propanoic Acid

To a solution of methyl 3-((2S,4R)-1-((S)-2-(1-fluorocyclopropanecarboxamido)-3,3-dimethylbutanoyl)-4-hydroxypyrrolidine-2-carboxamido)-3-(4-(4-methylthiazol-5-yl)phenyl)propanoate (1.80 g, 3.06 mmol) in THF (6 mL), MeOH (6 mL) and H₂O (6 mL) was added LiOH·H₂O (642 mg, 15.3 mmol). The mixture was then stirred at 25° C. for 12 h. On completion, the reaction mixture was poured into water (20 mL) and extracted with DCM (20 mL×3). The aqueous phase was acidified with 1N HCl to pH=4, then extracted with DCM (20 mL×3). The combined organic layers were washed with brine (20 mL×3), dried over Na₂SO₄, filtered and concentrated under reduced pressure to give the title compound (2.1 g) as a white solid. ¹H NMR (400 MHz, DMSO-d₆) δ 8.98 (d, J=2.4 Hz, 1H), 8.56 (br dd, J=8.4, 17.4 Hz, 1H), 7.53-7.48 (m, 1H), 7.46-7.38 (m, 3H), 7.26 (dt, J=2.4, 9.6 Hz, 1H), 5.23-5.09 (m, 2H), 4.57 (br dd, J=2.4, 9.2 Hz, 1H), 4.46 (td, J=8.4, 18.4 Hz, 1H), 4.34 (br s, 1H), 4.26 (br s, 1H), 3.66-3.51 (m, 2H), 2.83-2.80 (m, 1H), 2.78 (br d, J=5.6 Hz, 1H), 2.74-2.61 (m, 1H), 2.45 (d, J=3.2 Hz, 3H), 2.08-1.99 (m, 1H), 1.90 (ddd, J=4.8, 8.4, 12.8 Hz, 1H), 1.78-1.69 (m, 1H), 1.43-1.29 (m, 2H), 1.21 (br d, J=8.4 Hz, 2H), 0.98-0.94 (m, 5H), 0.93-0.89 (m, 5H).

### Step 7—(S)-3-((2S,4R)-1-((S)-2-(1-fluorocyclopropanecarboxamido)-3,3-dimethylbutanoyl)-4-hydroxypyrrolidine-2-carboxamido)-3-(4-(4-methylthiazol-5-yl)phenyl)propanoic Acid and (R)-3-((2S,4R)-1-((S)-2-(1-fluorocyclopropanecarboxamido)-3,3-dimethylbutanoyl)-4-hydroxypyrrolidine-2-carboxamido)-3-(4-(4-methylthiazol-5-yl)phenyl)propanoic Acid

3-((2S,4R)-1-((S)-2-(1-fluorocyclopropanecarboxamido)-3,3-dimethylbutanoyl)-4-hydroxypyrrolidine-2-carboxamido)-3-(4-(4-methylthiazol-5-yl)phenyl)propanoic acid (200 mg) was purified by prep-HPLC (column: REGIS (s,s) WHELK-O1 (250 mm*50 mm, 10 um); mobile phase: [0.1% NH₃H2O IPA]; B %: 60%-60%, 3; 30 min) to give desired compound as a white solid. This compound was further separated by SFC (Column: (S,S) Whelk-O1 50×4.6 mm I.D., 1.8 um, mobile phase: Phase A for CO2, and Phase B for IPA (0.05% DEA); gradient elution: B in A from 5% to 40 low rate: 2.5 mL/min; Detector: PDA: column Temp: 35C; Back Pressure: 100 Bar) to give(S)-3-((2S,4R)-1-((S)-2-(1-fluorocyclopropanecarboxamido)-3,3-dimethylbutanoyl)-4-hydroxypyrrolidine-2-carboxamido)-3-(4-(4-methylthiazol-5-yl)phenyl)propanoic acid (79.29 mg, 4% yield) as a white solid (¹H NMR (400 MHz, DMSO-d₆) δ 12.56-12.06 (m, 1H), 8.98 (s, 1H), 8.58 (br d, J=8.4 Hz, 1H), 7.53-7.46 (m, 2H), 7.40 (d, J=8.4 Hz, 2H), 7.24 (dd, J=2.4, 9.2 Hz, 1H), 5.23-5.12 (m, 2H), 4.56 (d, J=9.2 Hz, 1H), 4.48 (t, J=8.0 Hz, 1H), 4.34 (br s, 1H), 3.67-3.60 (m, 1H), 3.59-3.54 (m, 1H), 2.83-2.75 (m, 1H), 2.68-2.61 (m, 1H), 2.45 (s, 3H), 2.07-1.97 (m, 1H), 1.90 (ddd, J=4.8, 8.4, 13.2 Hz, 1H), 1.39-1.31 (m, 2H), 1.24-1.19 (m, 2H), 0.91 (s, 9H). LC-MS (ESI⁺) m/z 575.2 (M+H)⁺) and (R)-3-((2S,4R)-1-((S)-2-(1-fluorocyclopropanecarboxamido)-3,3-dimethylbutanoyl)-4-hydroxypyrrolidine-2-carboxamido)-3-(4-(4-methylthiazol-5-yl)phenyl)propanoic acid (71.8 mg, 4% yield) as a white solid (¹H NMR (400 MHz, DMSO-d₆) δ=12.43-12.09 (m, 1H), 8.99 (s, 1H), 8.53 (d, J=8.0 Hz, 1H), 7.47-7.37 (m, 4H), 7.27 (dd, J=2.6, 9.4 Hz, 1H), 5.16-5.10 (m, 2H), 4.57 (d, J=9.2 Hz, 1H), 4.43 (t, J=8.4 Hz, 1H), 4.28-4.24 (m, 1H), 3.63-3.50 (m, 2H), 2.84-2.77 (m, 1H), 2.73-2.67 (m, 1H), 2.46 (s, 3H), 2.08-1.99 (m, 1H), 1.73 (ddd, J=4.4, 8.8, 12.8 Hz, 1H), 1.43-1.37 (m, 1H), 1.36-1.29 (m, 1H), 1.24-1.19 (m, 2H), 0.98-0.94 (m, 9H) LC-MS (ESI⁺) m/z 575.2 (M+H)⁺). The absolute stereochemistry of the diastereomers was assigned arbitrarily.

### Synthesis of 6-((Tert-butoxycarbonyl)(methyl)amino)hexanoic Acid (Intermediate TB)

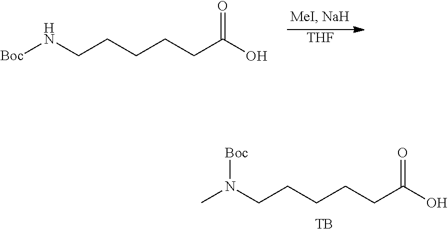

To a solution of 6-(tert-butoxycarbonylamino)hexanoic acid (4.0 g, 17 mmol) in THF (40 mL) was added NaH (2.77 g, 69.2 mmol, 60% dispersion in mineral oil) at 0° C., then the mixture was stirred for 0.5 h. Next, MeI (7.36 g, 51.8 mmol, 3.23 mL) was added and the mixture was stirred at 25° C. for 12 h. On completion, the reaction mixture was poured into aq. saturated NH₄Cl (100 mL) to quench, then extracted with EtOAc (100 mL×3). The combined organic layers were washed with brine (100 mL×2), dried over Na₂SO₄, filtered and concentrated under reduced pressure to give the title compound (3.8 g) as yellow gum.

### Synthesis of (R)-4-(4-((4′-chloro-4,4-dimethyl-3,4,5,6-tetrahydro-[1,1′-biphenyl]-2-yl)methyl)piperazin-1-yl)-N-((4-((4-(4-(6-(methylamino)hexanoyl)piperazin-1-yl)-1-(phenylthio)butan-2-yl)amino)-3-((trifluorom ethyl)sulfonyl)phenyl)sulfonyl)benzamide (Intermediate TC)

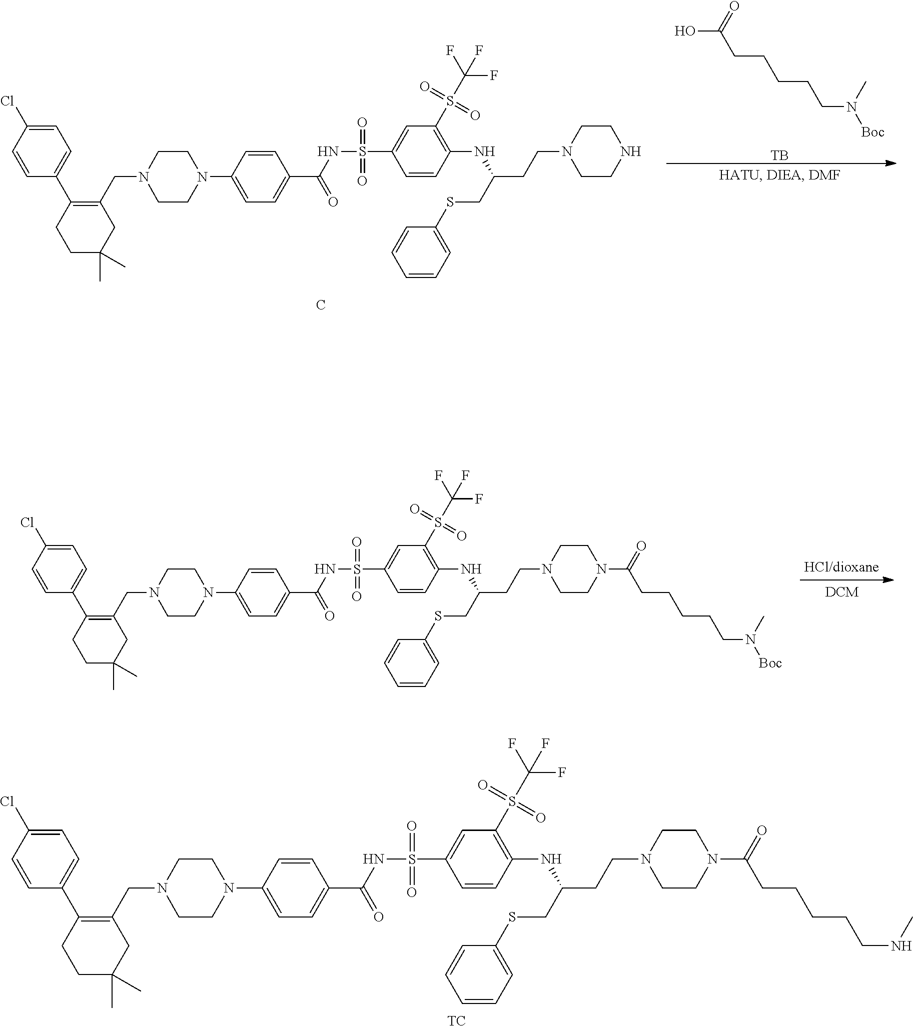

### Step 1—(R)-tert-butyl (6-(4-(3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-6-oxohexyl)(methyl)carbamate

A mixture of (R)-4-(4-((4′-chloro-4,4-dimethyl-3,4,5,6-tetrahydro-[1,1′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide (500 mg, 513 μmol, Intermediate C), 6-((tert-butoxycarbonyl)(methyl)amino)hexanoic acid (151 mg, 616 μmol, Intermediate TB), DIEA (265 mg, 2.05 mmol, 358 uL) in DMF (5 mL) was added HATU (205 mg, 539 μmol), and then the mixture was stirred at 25° C. for 12 h. On completion, the reaction mixture was quenched by addition with ice water (5 mL) at 20° C., and extracted with EtOAc (5 mL×4). The combined organic layers were washed with water (5 mL× 2) and washed with brine (5 mL×2), dried over Na₂SO₄, filtered and concentrated under reduced pressure to give a residue. The residue was purified by column chromatography (SiO₂, Petroleum ether/Ethyl acetate=1/1 to 0/1) to give the title compound (319 mg, 52% yield) as a white solid. ¹H NMR (400 MHz, DMSO-d₆) δ=8.12 (d, J=2.0 Hz, 1H), 7.94 (dd, J=2.0, 9.2 Hz, 1H), 7.71 (d, J=8.8 Hz, 2H), 7.38 (s, 1H), 7.36 (s, 1H), 7.34 (s, 1H), 7.32 (s, 1H), 7.26 (t, J=7.6 Hz, 2H), 7.19-7.15 (m, 1H), 7.13 (s, 1H), 7.11 (s, 1H), 6.99 (br d, J=9.6 Hz, 1H), 6.91-6.84 (m, 3H), 4.08 (br d, J=5.2 Hz, 1H), 3.28-3.19 (m, 7H), 3.11 (br t, J=7.2 Hz, 3H), 2.89 (s, 1H), 2.73 (s, 3H), 2.70-2.66 (m, 1H), 2.44-2.35 (m, 7H), 2.24-2.17 (m, 6H), 2.01-1.99 (m, 2H), 1.43 (br s, 8H), 1.38 (br s, 2H), 1.37 (s, 9H), 1.23-1.13 (m, 4H), 0.97 (s, 6H).

### Step 2—(R)-4-(4-((4′-chloro-4,4-dimethyl-3,4,5,6-tetrahydro-[1,1′-biphenyl]-2-yl)methyl)piperazin-1-yl)-N-((4-((4-(4-(6-(methylamino)hexanoyl)piperazin-1-yl)-1-(phenylthio)butan-2-yl)amino)-3-((trifluorom ethyl)sulfonyl)phenyl)sulfonyl)benzamide

A mixture of (R)-tert-butyl (6-(4-(3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)-6-oxohexyl)(methyl)carbamate (150 mg, 125 μmol) and HCl/dioxane (4 M, 0.3 mL) in DCM (3 mL) was stirred at 25° C. for 12 h. On completion, the reaction mixture was concentrated under reduced pressure to give the title compound (158 mg) as a white solid. ¹H NMR (400 MHz, DMSO-d₆) δ=8.18 (d, J=2.0 Hz, 1H), 7.99-7.94 (m, 1H), 7.77 (br d, J=8.8 Hz, 2H), 7.41 (d, J=8.4 Hz, 2H), 7.30 (s, 1H), 7.28 (s, 1H). 7.24 (s, 1H), 7.22 (s, 1H), 7.20-7.17 (m, 2H), 7.17-7.14 (m, 2H), 6.99-6.94 (m, 3H), 4.46-4.32 (m, 1H), 4.30-4.17 (m, 1H), 4.13-3.91 (m, 2H), 3.88 (br d, J=12 Hz, 2H), 3.47-3.38 (m, 8H), 3.16 (s, 2H), 2.89 (s, 1H), 2.84-2.72 (m, 6H), 2.69 (s, 1H), 2.38-2.25 (m, 6H), 2.18 (br s, 4H), 1.65-1.54 (m, 3H), 1.51-1.45 (m, 4H), 1.37-1.28 (m, 3H), 1.23 (s, 2H), 1.00 (s, 6H).

### Synthesis of 4-(4-((4′-Chloro-4,4-dimethyl-3,4,5,6-tetrahydro-[1,1′-biphenyl]-2-yl)methyl)piperazin-1-yl)-N-((4-fluoro-3-((trifluoromethyl)sulfonyl)phenyl)sulfonyl)benzamide (Intermediate TD)

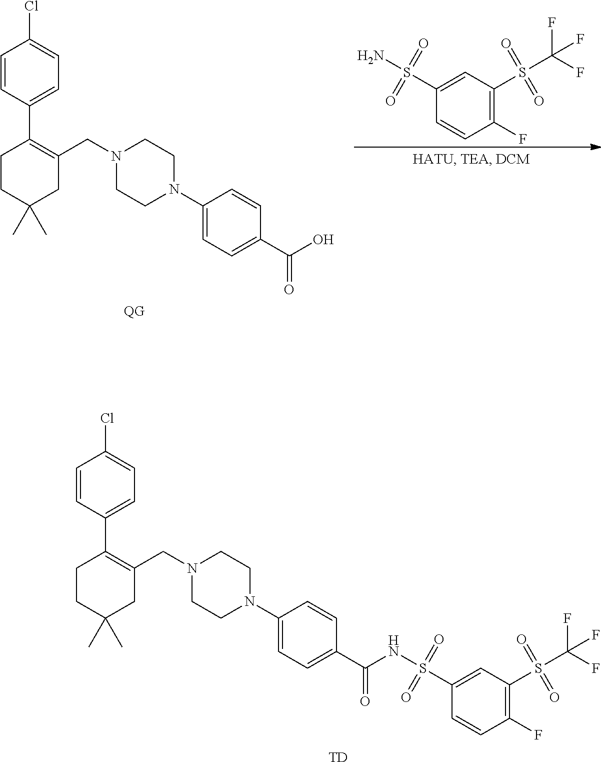 [SMILES: CC1(C)CCC(c2ccc(Cl)cc2)=C(CN2CCN(c3ccc(C(=O)O)cc3)CC2)C1 ; NS(=O)(=O)c1ccc(F)c(S(=O)(=O)C(F)(F)F)c1 ; CC1(C)CCC(c2ccc(Cl)cc2)=C(CN2CCN(c3ccc(C(=O)NS(=O)(=O)c4ccc(F)c(S(=O)(=O)C(F)(F)F)c4)cc3)CC2)C1 ; "TD"]

A mixture of 4-fluoro-3-((trifluoromethyl)sulfonyl)benzenesulfonamide (2.10 g, 6.83 mmol), 4-(4-((4′-chloro-4,4-dimethyl-3,4,5,6-tetrahydro-[1,1′-biphenyl]-2-yl)methyl)piperazin-1-yl)benzoic acid (3 g, 6.83 mmol, Intermediate QG), HATU (2.65 g, 6.97 mmol) and TEA (3.46 g, 34.2 mmol, 4.76 mL) in DCM (10 mL) was stirred at 25° C. for 12 h. On completion, the reaction was poured into water (20 mL) and extracted with DCM (15 mL×3). The combined organic phase is washed with brine (15 mL×3), dried over sodium sulfate, filtered and concentrated to give a residue. The residue was purified by column chromatography (SiO₂, Petroleum ether/Ethyl acetate=1/1 to 0:1) to give the title compound (2 g, 40% yield) as a white solid. LC-MS (ESI+) m/z 728.22 (M+H)⁺.

### Synthesis of Tert-butyl 4-(2-(tosyloxy)ethyl)piperidine-1-carboxylate (Intermediate TE)

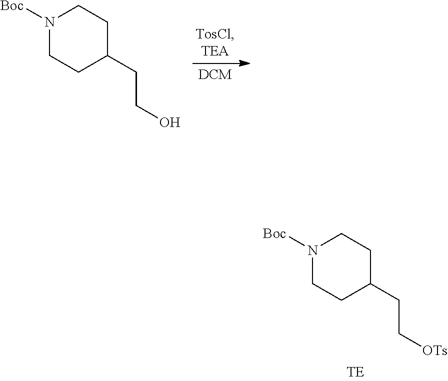 [SMILES: CC(C)(C)OC(=O)N1CCC(CCO)CC1 ; Cc1ccc(S(=O)(=O)OCCC2CCN(C(=O)OC(C)(C)C)CC2)cc1]

To a solution of tert-butyl 4-(2-hydroxyethyl)piperidine-1-carboxylate (1.0 g, 4.4 mmol) in DCM (10 mL) was added TEA (2.21 g, 21.8 mmol, 3.03 mL) and TosCl (1.08 g, 5.67 mmol). The mixture was then stirred at 0-25° C. for 1 hr. On completion, the reaction was diluted with EtOAc (10 mL) and water with HCl (2M, 0.5 ml) until the pH was 5˜6, then the mixture was extracted with EtOAc (15 mL×3). The combined organic phase is washed with brine (15 mL×3), dried over sodium sulfat, filtered and concentrated to give the title compound (1.6 g) as a white oil. LC-MS (ESI+) m/z 383.50 (M+H)⁺; ¹H NMR (400 MHz, DMSO-d₆) δ 7.79 (d, J=8.3 Hz, 2H), 7.49 (d, J=8.1 Hz, 2H), 4.04 (t, J=6.1 Hz, 2H), 3.84 (br d, J=12.4 Hz, 2H), 2.58 (br s, 2H), 2.42 (s, 3H), 1.49-1.39 (m, 4H), 1.38 (s, 1H), 1.37 (s, 9H), 0.96-0.80 (m, 2H).

### Synthesis of (2S,4R)-1-((S)-2-(1-fluorocyclopropanecarboxamido)-3,3-dimethylbutanoyl)-4-hydroxy-N-(4-(4-methylthiazol-5-yl)-2-(2-(piperidin-4-yl)ethoxy)benzyl)pyrrolidine-2-carboxamide (Intermediate TF)

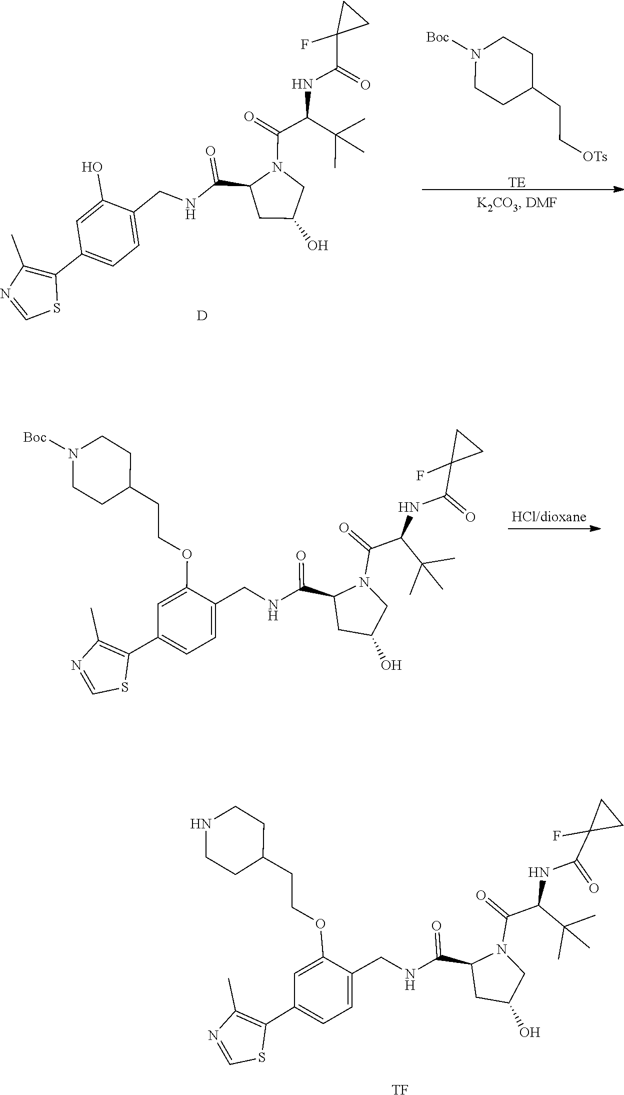

### Step 1—Tert-butyl 4-(2-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)ethyl)piperidine-1-carboxylate

To a solution of tert-butyl 4-(2-(tosyloxy)ethyl)piperidine-1-carboxylate (432 mg, 1.13 mmol, Intermediate TE) and (2S,4R)-1-((S)-2-(1-fluorocyclopropanecarboxamido)-3-methylbutanoyl)-4-hydroxy-N-(2-hydroxy-4-(4-methylthiazol-5-yl)benzyl)pyrrolidine-2-carboxamide (500 mg, 938 μmol, Intermediate D) in DMF (5 mL) was added K₂CO₃ (389 mg, 2.82 mmol). The mixture was then stirred at 70° C. for 5 h. On completion, the reaction was poured into water (20 mL) and extracted with EtOAc (15 mL×3). The combined organic phase is washed with brine (15 mL×3), dried over sodium sulfate, filtered and concentrated to give a residue. The residue was purified by prep-HPLC (column: Phenomenex Luna C18 200*40 mm*10 um; mobile phase: [water (TFA)-ACN]; B %: 43%-73%, 10 min) to give the title compound (550 mg, 78% yield) as a yellow solid. LC-MS (ESI+) m/z 743.93 (M+H)⁺; ¹H NMR (400 MHz, DMSO-d₆) δ 8.99 (s, 1H), 8.49 (t, J=5.9 Hz, 1H), 7.40 (d, J=7.9 Hz, 1H), 7.28 (dd, J=2.6, 9.3 Hz, 1H), 7.02 (d, J=1.1 Hz, 1H), 6.95 (dd, J=1.2, 7.8 Hz, 1H), 5.67-4.74 (m, 3H), 4.59 (d, J=9.1 Hz, 1H), 4.52 (t, J=8.2 Hz, 1H), 4.35 (br s, 1H), 4.32-4.17 (m, 2H), 4.09 (br t, J=5.4 Hz, 2H), 3.92 (br d, J=11.6 Hz, 2H), 3.69-3.55 (m, 2H), 2.82-2.61 (m, 2H), 2.46 (s, 3H), 2.09 (br dd, J=7.9, 12.6 Hz, 1H), 1.92 (ddd, J=4.5, 8.7, 12.9 Hz, 1H), 1.70 (br s, 5H), 1.39 (s, 9H), 1.25-1.19 (m, 2H), 1.11-1.00 (m, 2H), 0.95 (s, 9H).

### Step 2—(2S,4R)-1-((S)-2-(1-fluorocyclopropanecarboxamido)-3,3-dimethylbutanoyl)-4-hydroxy-N-(4-(4-methylthiazol-5-yl)-2-(2-(piperidin-4-yl)ethoxy)benzyl)pyrrolidine-2-carboxamide

To a solution of tert-butyl 4-(2-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)ethyl)piperidine-1-carboxylate (100 mg, 134 μmol) in DCM (1 mL) was added HCl/dioxane (4 M, 33.6 uL). The mixture was then stirred at 25° C. for 0.5 h. On completion, the reaction was filtered and concentrated under reduced pressure to the title compound (86.5 mg) as white solid. LC-MS (ESI+) m/z 643.32 (M+H)⁺.

### Synthesis of (2S,4R)—N-(2-(2-(1-((R)-3-amino-4-(phenylthio)butyl)piperidin-4-yl)ethoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (Intermediate TG)

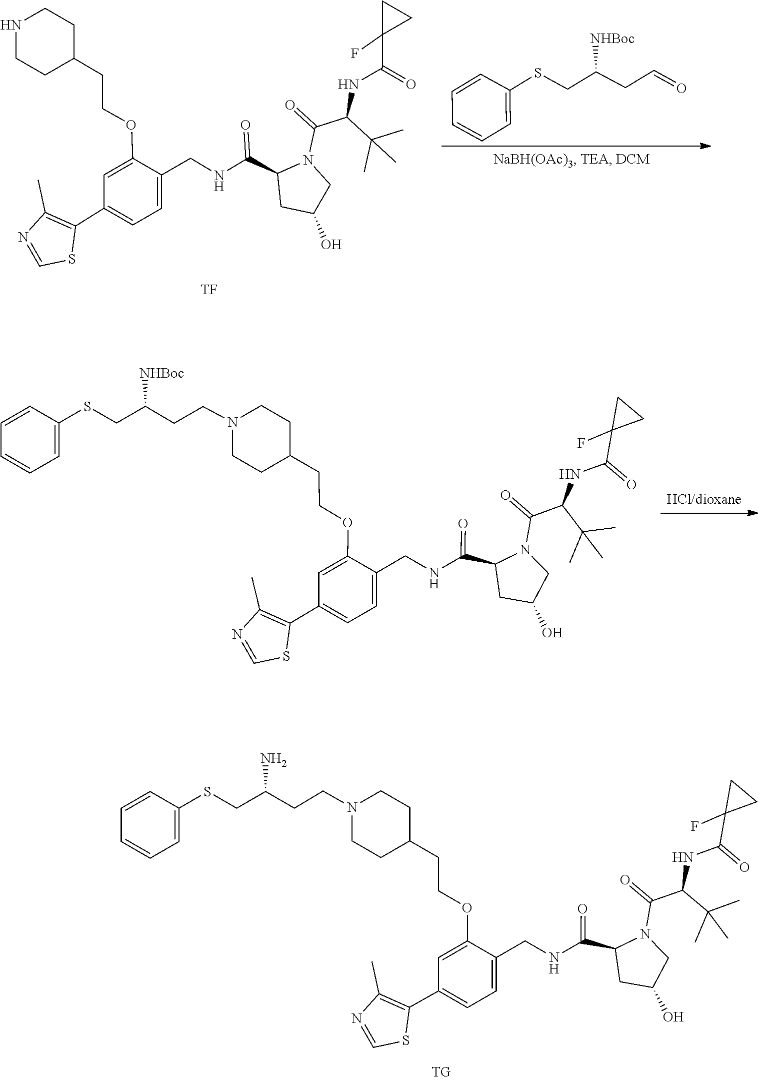

### Step 1—Tert-butyl ((R)-4-(4-(2-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)ethyl)piperidin-1-yl)-1-(phenylthio)butan-2-yl)carbamate

To a solution of (2S,4R)-1-((S)-2-(1-fluorocyclopropanecarboxamido)-3,3-dimethylbutanoyl)-4-hydroxy-N-(4-(4-methylthiazol-5-yl)-2-(2-(piperidin-4-yl)ethoxy)benzyl)pyrrolidine-2-carboxamide (63 mg, 92.6 μmol, HCl, Intermediate TF) and (R)-tert-butyl (4-oxo-1-(phenylthio)butan-2-yl)carbamate (27.4 mg, 92.6 μmol, synthesized via Step 1 of Intermediate KX) in DCM (1.5 mL) was added NaBH(OAc)₃ (29.4 mg, 139 μmol) at 0° C. and the mixture was stirred for 0.5 h. The mixture was then stirred at 25° C. for 2 h. On completion, the reaction was poured into water (20 mL) and extracted with DCM (15 mL×3). The combined organic phase is washed with brine (15 mL×3), dried over sodium sulfate, filtered and concentrated to give a residue. The residue was purified by column chromatography (SiO₂, Petroleum ether/Ethyl acetate=0/1 to DCM:MeOH=10:1) to give the title compound (60 mg, 70% yield) as a yellow gum. LC-MS (ESI⁺) m/z 923.21 (M+H)⁺.

### Step 2—(2S,4R)—N-(2-(2-(1-((R)-3-amino-4-(phenylthio)butyl)piperidin-4-yl)ethoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide

To a solution of tert-butyl ((R)-4-(4-(2-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)ethyl)piperidin-1-yl)-1-(phenylthio)butan-2-yl)carbamate (60 mg, 65 μmol) in DCM (1 mL) was added HCl/dioxane (4 M, 16.3 uL). The mixture was then stirred at 25° C. for 0.5 h. On completion, the reaction was filtered and concentrated under reduced pressure to give the title compound (50 mg) as a yellow gum. LC-MS (ESI⁺) m/z 823.09 (M+H)⁺.

### Synthesis of 3-((tert-butoxycarbonyl)(methyl)amino)propyl 4-methylbenzenesulfonate (Intermediate TH)

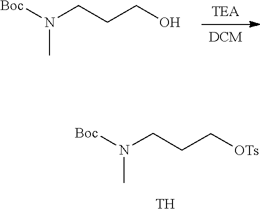

To a solution of tert-butyl N-(3-hydroxypropyl)-N-methyl-carbamate (2 g, 10.6 mmol, CAS #98642-44-5) and TosCl (3.02 g, 15.9 mmol) in DCM (20 mL) was added TEA (2.14 g, 21.1 mmol, 2.94 mL) at 0° C. The mixture was then stirred at 25° C. for 4 h. On completion, the mixture was quenched with H₂O (20 mL×2), and extracted with DCM (20 mL×2). The combined organic layer was washed with brine (20 mL), dried over anhydrous Na₂SO₄, filtered and concentrated under reduced pressure to give a residue. The residue was purified by column chromatography (SiO₂, Petroleum ether:Ethyl acetate=3:1 to Petroleum ether:Ethyl acetate=0:1) to give the title compound (1.5 g, 41% yield) as a white oil. LC-MS (ESI⁺) m/z 365.9 (M+Na)⁻.

### Synthesis of (2S,4R)-1-((S)-2-(1-fluorocyclopropanecarboxamido)-3,3-dimethylbutanoyl)-4-hydroxy-N-(2-(3-(methylamino)propoxy)-4-(4-methylthiazol-5-yl)benzyl)pyrrolidine-2-carboxamide (Intermediate TI)

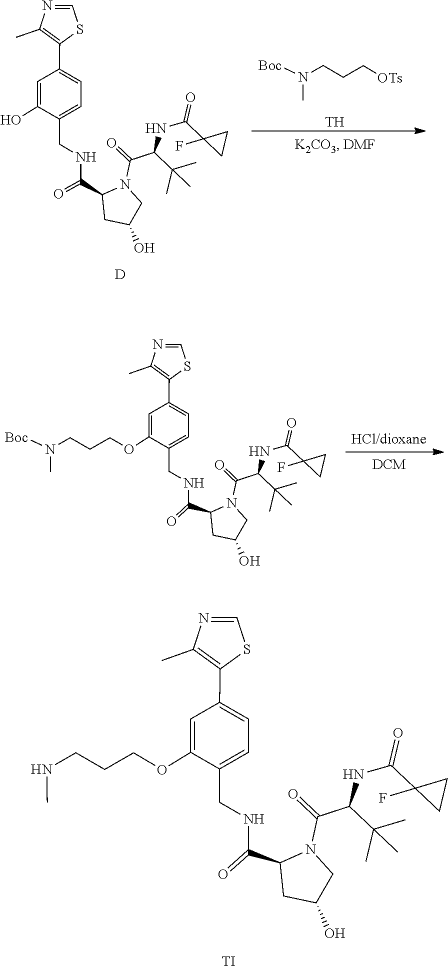

### Step 1—Tert-butyl (3-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)propyl)(methyl)carbamate

To a solution of 3-[tert-butoxycarbonyl(methyl)amino]propyl 4-methylbenzenesulfonate (389 mg, 1.13 mmol, Intermediate TH), (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (300 mg, 563 μmol, Intermediate D) in DMF (3 mL) was added K₂CO₃ (234 mg, 1.69 mmol). The mixture was then stirred at 70° C. for 12 hr. On completion, the crude product was purified by reversed-phase HPLC (0.1% FA condition) to give the title compound (150 mg, 38% yield) as a white solid. LC-MS (ESI⁺) m/z 704.3 (M+H)⁺.

### Step 2—(2S,4R)-1-((S)-2-(1-fluorocyclopropanecarboxamido)-3,3-dimethylbutanoyl)-4-hydroxy-N-(2-(3-(methylamino)propoxy)-4-(4-methylthiazol-5-yl)benzyl)pyrrolidine-2-carboxamide

To a solution of tert-butyl N-[3-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]propyl]-N-methyl-carbamate (150 mg, 213 μmol) in DCM (3 mL) was added HCl/dioxane (4 M, 53.3 uL). The mixture was then stirred at 25° C. for 1 hr. On completion, the mixture was concentrated in vacuo to give the title compound (120 mg, 93% yield) as a white solid. LC-MS (ESI⁺) m/z 604.6 (M+H)⁺.

### Synthesis of (2S,4R)—N-(2-(3-((3-amino-4-(phenylthio)butyl)(methyl)amino)propoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (Intermediate TJ)

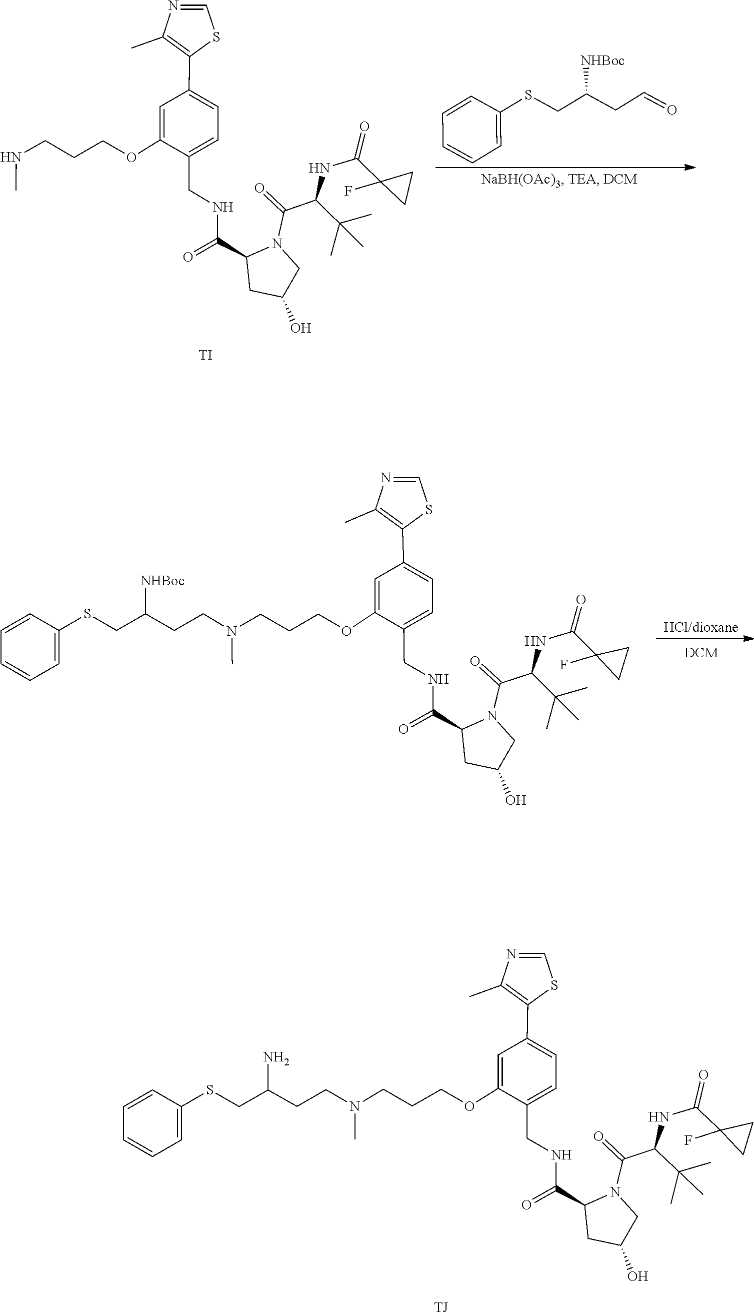

### Step 1—Tert-butyl (4-((3-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)propyl)(methyl)amino)-1-(phenylthio)butan-2-yl)carbamate

To a solution of tert-butyl N-[(1R)-3-oxo-1-(phenylsulfanylmethyl)propyl]carbamate (39.1 mg, 133 μmol, synthesized via Step 1 of Intermediate KX), (2S,4R)-1-[(2S)-2-[(1-fluorocyclopropanecarbonyl)amino]-3,3-dimethyl-butanoyl]-4-hydroxy-N-[[2-[3-(methylamino)propoxy]-4-(4-methylthiazol-5-yl)phenyl]methyl]pyrrolidine-2-carboxamide (80 mg, 133 μmol, Intermediate TI) in DCM (1 mL) was added TEA (53.6 mg, 530 μmol, 73.7 uL) at 25° C. Next, NaBH(OAc)₃ (42.1 mg, 199 μmol) was added at 0° C. The mixture was then stirred at 25° C. for 2 h. On completion, the mixture was washed with H₂O (1 mL×2), then with brine (2 mL×2), dried over anhydrous Na₂SO₄, filtered and concentrated under reduced pressure to give a residue. The residue was purified by column chromatography (SiO₂, Petroleum ether:Ethyl acetate=5:1 to Dichloromethane:Methanol=10:1) to give the title compound (46 mg, 39% yield) as a white solid. LC-MS (ESI⁺) m/z 780.7 (M-100)⁺.

### Step 2—(2S,4R)—N-(2-(3-((3-amino-4-(phenylthio)butyl)(methyl)amino)propoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide

To a solution of tert-butyl N-[(1R)-3-[3-[2-[[[(2S,4R)-1-[(2S)-2-[(1-fluorocyclopropanecarbonyl)amino]-3,3-dimethyl-butanoyl]-4-hydroxy-pyrrolidine-2-carbonyl]amino]methyl]-5-(4-methylthiazol-5-yl)phenoxy]propyl-methyl-amino]-1-(phenylsulfanylmethyl)propyl]carbamate (46 mg, 52.09 μmol) in DCM (0.5 mL) was added HCl/dioxane (4 M, 22.6 uL). The mixture was then stirred at 25° C. for 1 h. On completion, the mixture was concentrated in vacuo to give the title compound (40 mg) as a white solid. LC-MS (ESI⁺) m/z 783.5 (M+H)⁺; ¹H NMR (400 MHz, DMSO-d₆) δ=13.16-12.67 (m, 1H), 8.98 (s, 1H), 8.38 (br d, J=7.2 Hz, 1H), 8.14 (s, 1H), 8.03 (d, J=8.0 Hz, 1H), 7.79 (d, J=8.0 Hz, 1H), 7.67 (d, J=7.2 Hz, 1H), 7.50-7.32 (m, 9H), 7.29-7.23 (m, 1H), 7.21-7.15 (m, 1H), 7.14-7.06 (m, 1H), 5.69 (br d, J=8.4 Hz, 1H), 5.09 (br d, J=2.4 Hz, 1H), 4.91 (br t, J=6.8 Hz, 1H), 4.83 (s, 2H), 4.42 (t, J=8.0 Hz, 1H), 4.36 (br d, J=9.2 Hz, 1H), 4.27 (br s, 1H), 4.07 (br t, J=6.4 Hz, 2H), 3.98-3.86 (m, 2H), 3.71 (br t, J=6.0 Hz, 2H), 3.66-3.55 (m, 2H), 3.45 (s, 2H), 3.16 (br t, J=7.2 Hz, 3H), 3.02 (br t, J=5.6 Hz, 2H), 2.65 (br s, 3H), 2.45 (s, 4H), 2.43-2.35 (m, 4H), 2.11 (br s, 3H), 2.06-1.96 (m, 4H), 1.82-1.75 (m, 1H), 1.72-1.56 (m, 4H), 1.36 (br d, J=7.2 Hz, 3H), 0.93 (s, 9H).

### Synthesis of 4-[4-[[2-(4-Chlorophenyl)-5,5-dimethyl-cyclohexen-1-yl]methyl]piperazin-1-yl]-N-[4-[[(1R)-1-(phenylsulfanylmethyl)-3-piperazin-1-yl-propyl]amino]-3-(trifluoromethylsulfonyl)phenyl]sulfonyl-benzamide (CAS #2143096-93-7) (Intermediate TK)

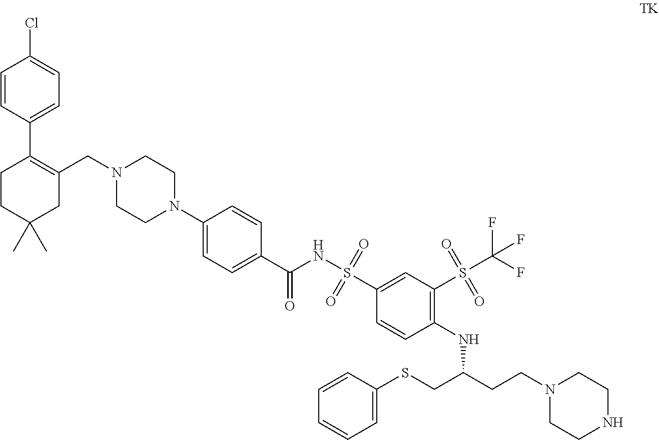 [SMILES: CC1(C)CCC(c2ccc(Cl)cc2)=C(CN2CCN(c3ccc(C(=O)NS(=O)(=O)c4ccc(N[C@H](CCN5CCNCC5)CSc5ccccc5)c(S(=O)(=O)C(F)(F)F)c4)cc3)CC2)C1]

### Synthesis of (2S,4R)—N-[[2-acetonyloxy-4-(4-methylthiazol-5-yl)phenyl]methyl]-1-[(2S)-2-[(1-fluorocyclopropanecarbonyl)amino]-3,3-dimethyl-butanoyl]-4-hydroxy-pyrrolidine-2-carboxamide (Intermediate TL)

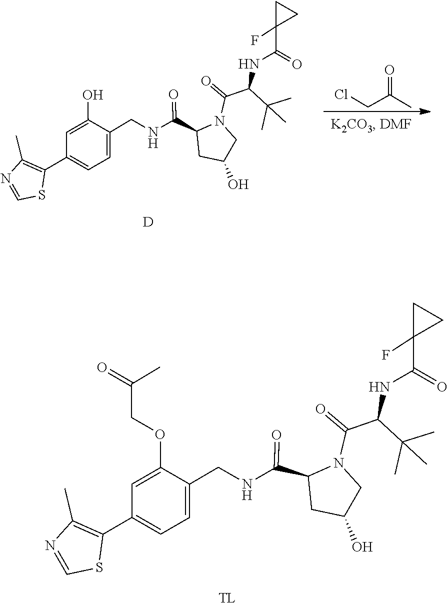

To a solution of (2S,4R)-1-[(2S)-2-[(1-fluorocyclopropanecarbonyl)amino]-3,3-dimethyl-butanoyl]-4-hydroxy-N-[[2-hydroxy-4-(4-methylthiazol-5-yl)phenyl]methyl]pyrrolidine-2-carboxamide (200 mg, 375 μmol, Intermediate D) in DMF (2 mL) was added K₂CO₃ (156 mg, 1.13 mmol), and the mixture was stirred at 25° C. for 10 mins. Then 1-chloropropan-2-one (110 mg, 1.19 mmol) was added and the reaction mixture was stirred at 25° C. for 2 hrs. On completion, the reaction mixture was diluted with EtOAc (100 mL), the mixture was washed with brine (50 mL×3), dried over Na₂SO₄, filtered and concentrated in vacuo to give the title compound (200 mg, 90% yield) as white solid. ¹H NMR (400 MHz, DMSO-d₆) δ 8.98 (s, 1H), 8.53 (t, J=5.6 Hz, 1H), 7.43 (d, J=7.6 Hz, 1H), 7.32-7.26 (m, 1H), 6.97 (d, J=8.4 Hz, 1H), 6.90 (s, 1H), 5.17 (d, J=3.6 Hz, 1H), 4.92 (s, 2H), 4.60 (d, J=9.2 Hz, 1H), 4.52 (t, J=8.4 Hz, 1H), 4.40-4.24 (m, 3H), 3.69-3.58 (m, 2H), 2.42 (s, 3H), 2.21-2.19 (m, 3H), 2.13-2.06 (m, 1H), 1.92 (ddd, J=4.4, 8.8, 12.8 Hz, 1H), 1.41-1.32 (m, 2H), 1.22 (dd, J=2.8, 8.4 Hz, 2H), 0.96 (s, 9H).

### Synthesis of Tert-butyl 3-(2-(tosyloxy)ethyl)azetidine-1-carboxylate (Intermediate TM)

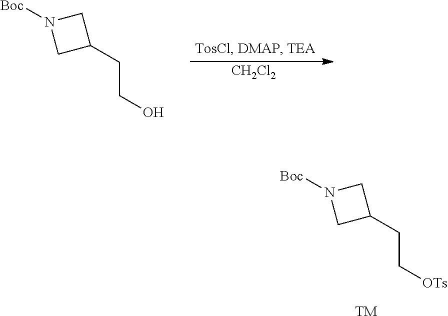

To a solution of tert-butyl 3-(2-hydroxyethyl)azetidine-1-carboxylate (1.00 g, 4.97 mmol, CAS #152537-03-6), DMAP (60.7 mg, 496 μmol), and TEA (1.51 g, 14.9 mmol) in CH₂Cl₂ (10 mL) was added to TosCl (1.14 g, 5.96 mmol) under 0° C., then the mixture was stirred at 25° C. for 12 h. On completion, the reaction mixture was quenched by addition of H₂O (5 mL), and then extracted with DCM (10 mL×3). The combined organic layers were washed with brine (10 mL×3), dried over Na₂SO₄, filtered and concentrated under reduced pressure to give a residue. The residue was purified by column chromatography (SiO₂, Petroleum ether/Ethyl acetate=3/1) to give the title compound (1.50 g, 84.9% yield) as a yellow oil. ¹H NMR (400 MHz, DMSO-d₆) δ=7.86 (d, J=8.4 Hz, 2H), 7.56 (d, J=8.0 Hz, 2H), 3.85 (t, J=8.0 Hz, 2H), 3.50 (s, 2H), 2.60-2.51 (m, 1H), 2.06 (s, 1H), 1.97-1.88 (m, 2H), 1.43 (s, 9H).

### Synthesis of (2S,4R)—N-(2-(2-(azetidin-3-yl)ethoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (Intermediate TN)

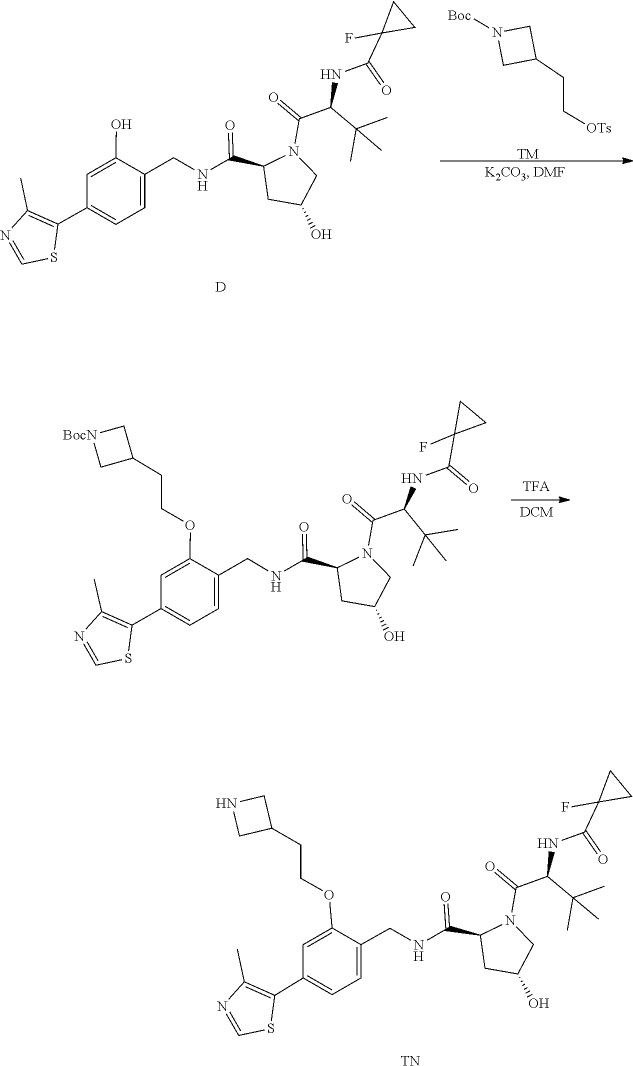

### Step 1—Tert-butyl 3-(2-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)ethyl)azetidine-1-carboxylate

To a solution of tert-butyl 3-(2-(tosyloxy)ethyl)azetidine-1-carboxylate (400 mg, 1.13 mmol, Intermediate™) and (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (500 mg, 938 μmol, Intermediate D) in DMF (5 mL) was added to K₂CO₃ (389 mg, 2.82 mmol), then the mixture was stirred at 60° C. for 2 h. On completion, the reaction mixture was quenched by addition of sat. NH₄Cl (15 mL), and then extracted with EtOAc (10 mL×3). The combined organic layers were washed with brine (10 mL×3), dried over Na₂SO₄, filtered and concentrated under reduced pressure to give a residue. The residue was purified by column chromatography (SiO₂, DCM:MeOH=10:1) to give the title compound (550 mg, 82% yield) as a yellow oil. ¹H NMR (400 MHz, DMSO-d₆) δ=8.98 (s, 1H), 8.51 (t, J=6.0 Hz, 1H), 7.41 (d, J=7.6 Hz, 1H), 7.29 (dd, J=2.8, 9.2 Hz, 1H), 7.00-6.89 (m, 2H), 5.17 (d, J=3.6 Hz, 1H), 4.63-4.57 (m, 1H), 4.52 (t, J=8.0 Hz, 1H), 4.38-4.23 (m, 2H), 4.08-4.01 (m, 2H), 4.00-3.90 (m, 2H), 3.65-3.56 (m, 4H), 2.46-2.44 (m, 3H), 2.10-2.00 (m, 3H), 1.41-1.32 (m, 12H), 1.24-1.20 (m, 2H), 0.96 (s, 9H).

### Step 2—(2S,4R)—N-(2-(2-(azetidin-3-yl)ethoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide

To a solution of tert-butyl 3-(2-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)ethyl)azetidine-1-carboxylate (400 mg, 558 μmol) in CH₂Cl₂ (4 mL) was added TFA (14.8 g, 130 mmol), and the mixture was stirred at 25° C. for 2 h. On completion, the reaction mixture was quenched by addition of NaOH (5 mL, 15% solution) until the pH was 8˜9, and then extracted with DCM (5 mL×3). The combined organic layers were washed with brine (5 mL×3), dried over Na₂SO₄, filtered and concentrated under reduced pressure to give the title compound (300 mg) as a brown solid. LC-MS (ESI⁺) m/z 616.2 (M+H)⁺.

### Synthesis of (2S,4R)—N-(2-(2-(1-((R)-3-amino-4-(phenylthio)butyl)azetidin-3-yl)ethoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (Intermediate TO)

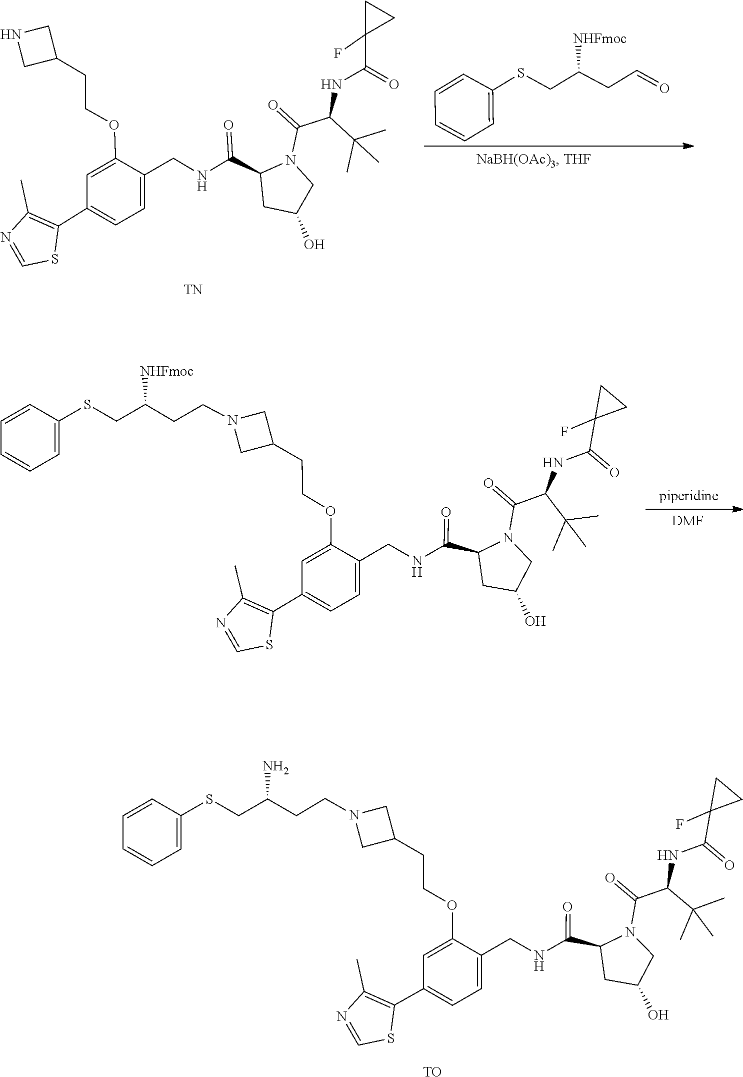

### Step 1—(9H-fluoren-9-yl)methyl ((R)-4-(3-(2-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)ethyl)azetidin-1-yl)-1-(phenylthio)butan-2-yl)carbamate

A mixture of (2S,4R)—N-(2-(2-(azetidin-3-yl)ethoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (300 mg, 487 μmol, Intermediate TN), (R)-(9H-fluoren-9-yl)methyl (4-oxo-1-(phenylthio)butan-2-yl)carbamate (290 mg, 487 μmol, synthesized via Steps 1-3 of Intermediate KK), and NaBH(OAc)₃ (206 mg, 974 μmol) in THF (3 mL) was stirred at 25° C. for 2 h. On completion, the reaction mixture was quenched by addition of H₂O (5 mL), and then extracted with EtOAc (5 mL×3). The combined organic layers were washed with brine (5 mL×3), dried over Na₂SO₄, filtered and concentrated under reduced pressure to give the title compound (450 mg) as a yellow solid. LC-MS (ESI⁺) m/z 509.4 (½M+H)⁺.

### Step 2—(2S,4R)—N-(2-(2-(1-((R)-3-amino-4-(phenylthio)butyl)azetidin-3-yl)ethoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide

To a solution of (9H-fluoren-9-yl)methyl ((R)-4-(3-(2-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)ethyl)azetidin-1-yl)-1-(phenylthio)butan-2-yl)carbamate (450 mg, 442 μmol) in DMF (3 mL) was added piperidine (1.94 g, 22.7 mmol). The mixture was stirred at 25° C. for 2 h. On completion, the reaction mixture was quenched by addition of H₂O (5 mL), and then extracted with EtOAc (5 mL×3). The combined organic layers were washed with brine (5 mL×3), dried over Na₂SO₄, filtered and concentrated under reduced pressure to give a residue. The crude product was purified by reversed-phase HPLC (20% ACN/H2O, 0.1% FA) to give the title compound (110 mg, 28% yield) as a brown solid. LC-MS (ESI⁺) m/z 795.5 (½M+H)⁺.

### Synthesis of (2S,4R)-1-[(2S)-2-[(1-fluorocyclopropanecarbonyl)amino]-3,3-dimethyl-butanoyl]-4-hydroxy-pyrrolidine-2-carboxylic Acid (Intermediate TP)

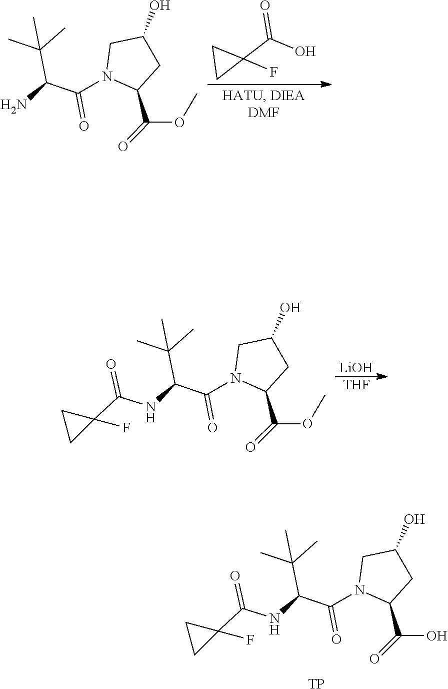

### Step 1—(2S,4R)-methyl 1-((S)-2-(1-fluorocyclopropanecarboxamido)-3,3-dimethylbutanoyl)-4-hydroxypyrrolidine-2-carboxylate

To a solution of methyl (2S,4R)-1-[(2S)-2-amino-3,3-dimethyl-butanoyl]-4-hydroxy-pyrrolidine-2-carboxylate (129 g, 437 mmol, CAS #1024616-23-6) in DMF (1500 mL) was added DIEA (169.68 g, 1.31 mol, 228.67 mL), HATU (216.32 g, 568.91 mmol), and 1-fluorocyclopropanecarboxylic acid (50.10 g, 481.39 mmol, CAS #137081-41-5). The mixture was then stirred at 25° C. for 2 hrs. On completion, the reaction mixture was quenched by addition of H₂O (500 mL) at 25° C., and then diluted with ethyl acetate (500 mL) and extracted with ethyl acetate (500 mL×3). The combined organic layers were washed with sat. NaCl (500 mL), dried over Na₂SO₄, filtered and concentrated under reduced pressure to give a residue. The residue was purified by column chromatography (SiO₂, Petroleum ether/Ethyl acetate=2/1) to give title compound (152 g, 99% yield) as a yellow oil. LC-MS (ESI⁺) m/z 345.0 (M+H)⁺.

### Step 2—(2S,4R)-1-((S)-2-(1-fluorocyclopropanecarboxamido)-3,3-dimethylbutanoyl)-4-hydroxypyrrolidine-2-carboxylic Acid

To a solution of methyl (2S,4R)-1-[(2S)-2-[(1-fluorocyclopropanecarbonyl)amino]-3,3-dimethyl-butanoyl]-4-hydroxy-pyrrolidine-2-carboxylate (30 g, 87 mmol) in THF (190 mL) and H₂O (44 mL) was added LiOH·H₂O (10.97 g, 261.3 mmol). The mixture was then stirred at 25° C. for 2 hrs. On completion, 2M HCl was added to the reaction mixture until pH=1 and extracted with ethyl acetate (300 mL×3). The combined organic layers were washed with sat. NaCl (500 mL), dried over Na₂SO₄, filtered and concentrated under reduced pressure to give the title compound (31 g) was a white solid. LC-MS (ESI⁺) m/z 331.4 (M+H)⁺.

### Synthesis of Tert-butyl N-[[4-(4-methylthiazol-5-yl)-2-(3-piperazin-1-ylpropyl)phenyl]methyl]carbamate (Intermediate TO)

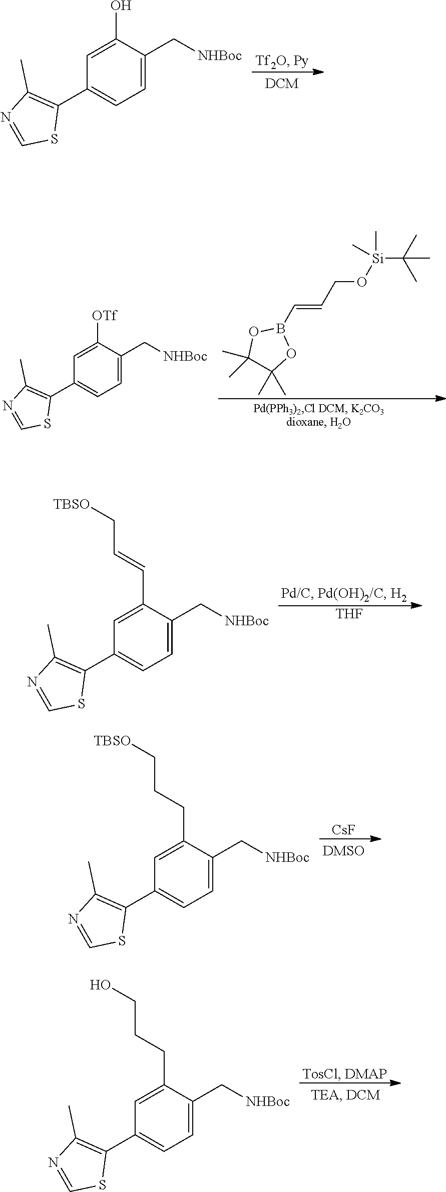

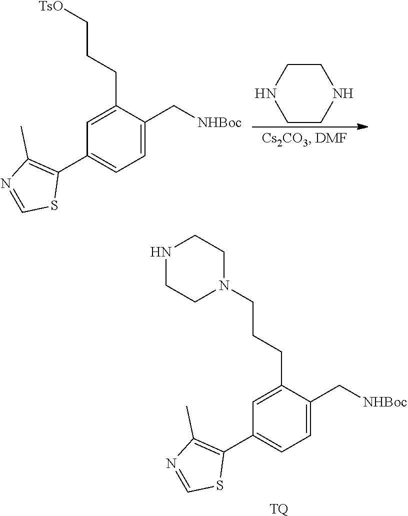

### Step 1—[2-[(Tert-butoxycarbonylamino)methyl]-5-(4-methylthiazol-5-yl)phenyl]trifluoromethanesulfonate

To a solution of tert-butyl N-[[2-hydroxy-4-(4-methylthiazol-5-yl)phenyl]methyl]carbamate (1.2 g, 3.75 mmol, CAS #1448190-11-1) in DCM (12 mL) was added Tf₂O (1.59 g, 5.62 mmol) and pyridine (592 mg, 7.49 mmol). The mixture was then stirred at −10° C. for 2 h. On completion, the reaction was diluted with DCM (50 mL) and extracted with DCM (80 mL×3). The combined organic layers were washed with water (50 mL×3), dried over Na₂SO₄, filtered and concentrated in vacuo to give the title compound (1.68 g, 99% yield) as a brown oil. LC-MS (ESI⁻) m/z 453.6 (M+H)⁺; ¹H NMR (400 MHz, DMSO-d6) δ 9.07 (s, 1H), 7.64 (dd, J=1.2, 8.0 Hz, 1H), 7.61-7.52 (m, 2H), 7.51 (d, J=1.6 Hz, 1H), 4.27 (d, J=6.0 Hz, 2H), 2.47 (s, 3H), 1.40 (s, 9H).

### Step 2—Tert-butyl N-[[2-[(E)-3-[tert-butyl(dimethyl)silyl]oxyprop-1-enyl]-4-(4-methylthiazol-5-yl)phenyl]methyl]carbamate

A mixture of [2-[(tert-butoxycarbonylamino)methyl]-5-(4-methylthiazol-5-yl)phenyl]trifluoromethanesulfonate (1.1 g, 2.43 mmol), tert-butyl-dimethyl-[(E)-3-(4,4,5,5-tetramethyl-1,3,2-dioxaborolan-2-yl)allyloxy]silane (1.09 g, 3.65 mmol, CAS #114653-19-9), K₂CO₃ (1.01 g, 7.29 mmol) and dichloropalladium triphenylphosphane (170 mg, 243 μmol) in dioxane (10 mL) and H₂O (2 mL) was degassed and purged with N₂ three times, and then the mixture was stirred at 80° C. for 3 h under N₂ atmosphere. On completion, the reaction mixture was filtered and concentrated in vacuo. The residue was diluted with water (30 mL) and extracted with EtOAc (30 mL×2). The combined organic layers was dried over Na₂SO₄, filtered and concentrated in vacuo to give a residue The residue was purified by column chromatography (SiO₂, PE:EtOAc=5:1˜1:1, PE:EtOAc=1:1, P1:Rf=0.25) to give the title compound (950 mg, 82% yield) as a yellow oil. LC-MS (ESI⁺) m/z 476.0 (M+H)⁺; ¹H NMR (400 MHz, DMSO-d₆) δ 8.99 (s, 1H), 7.53 (d, J=1.8 Hz, 1H), 7.36 (dd, J=1.6, 7.6 Hz, 2H), 7.33-7.29 (m, 1H), 6.88 (d, J=15.6 Hz, 1H), 6.30 (td, J=5.2, 15.6 Hz, 1H), 4.34 (dd, J=1.6, 5.2 Hz, 2H), 4.20 (d, J=6.0 Hz, 2H), 2.46 (s, 3H), 1.40 (s, 9H), 0.92-0.90 (m, 9H), 0.12-0.08 (m, 6H).

### Step 3—Tert-butyl N-[[2-[3-[tert-butyl(dimethyl)silyl]oxypropyl]-4-(4-methylthiazol-5-yl)phenyl]methyl]carbamate

A mixture of tert-butyl N-[[2-[(E)-3-[tert-butyl(dimethyl)silyl]oxyprop-1-enyl]-4-(4-methylthiazol-5-yl)phenyl]methyl]carbamate (950 mg, 2.00 mmol), Pd/C (300 mg, 2.00 mmol) and Pd(OH)₂ (300 mg, 213 μmol) in THF (10 mL) was degassed and purged with H₂ three times, and then the mixture was stirred at 25° C. for 2 hrs under H₂ (15 Psi) atmosphere. On completion, the reaction mixture was filtered and concentrated in vacuo to give the title compound (890 mg, 93% yield) as a white oil. ¹H NMR (400 MHz, DMSO-d₆) δ 8.97 (s, 1H), 7.35 (t, J=5.6 Hz, 1H), 7.29 (s, 2H), 7.24 (s, 1H), 4.19 (d, J=5.9 Hz, 2H), 3.62 (t, J=6.0 Hz, 2H), 2.72-2.65 (m, 2H), 2.44 (s, 3H), 1.78-1.67 (m, 2H), 1.41-1.35 (m. 9H), 0.87 (s, 9H), 0.03 (s, 6H).

### Step 4—Tert-butyl N-[[2-(3-hydroxypropyl)-4-(4-methylthiazol-5-yl)phenyl]methyl]carbamate

To a solution of tert-butyl N-[[2-[3-[tert-butyl(dimethyl)silyl]oxypropyl]-4-(4-methylthiazol-5-yl)phenyl]methyl]carbamate (860 mg, 1.80 mmol) in DMSO (5 mL) was added CsF (548 mg, 3.61 mmol). The mixture was then stirred at 25° C. for 20 hrs. On completion, the reaction was diluted with water (30 mL) and extracted with EtOAc (2×30 mL). The combined organic layers were dried over Na₂SO₄, filtered and concentrated in vacuo to give a residue. The residue was purified by column chromatography (SiO₂, PE:EtOAc=3:1 to 1:1, PE:EtOAc=0:1, P1:Rf=0.4) to give the title compound (520 mg, 79% yield) as a white oil. LC-MS (ESI⁺) m/z 363.6 (M+H)⁺; ¹H NMR (400 MHz, DMSO-d₆) δ 9.02 (s, 1H), 7.42 (s, 1H), 7.35-7.27 (m, 3H), 4.24 (d, J=5.6 Hz, 2H), 3.50 (q, J=6.0 Hz, 2H), 2.72 (t, J=7.6 Hz, 2H), 2.55 (d, J=1.2 Hz, 3H), 1.80-1.69 (m, 2H), 1.45 (s, 9H).

### Step 5—3-[2-[(Tert-butoxycarbonylamino)methyl]-5-(4-methylthiazol-5-yl)phenyl]propyl 4-methylbenzenesulfonate

To a solution of tert-butyl N-[2-(3-hydroxypropyl)-4-(4-methylthiazol-5-yl)phenyl methyl]carbamate (340 mg, 937 μmol), TEA (474 mg, 4.69 mmol) and DMAP (22.9 mg, 187 μmol) in DCM (5 mL) was added TosCl (232 mg, 1.22 mmol). The mixture was then stirred at 25° C. for 2 hrs. On completion, The reaction mixture was concentrated in vacuo, the residue was diluted with EtOAc (30 mL) and water (30 mL), then the residue was extracted with EtOAc (2×30 mL). The combined organic layer was dried over Na₂SO₄, filtered and concentrated in vacuo to give the title compound (480 mg, 99% yield) as a white oil. ¹H NMR (400 MHz, DMSO-d₆) δ 8.98 (s, 1H), 7.79 (d, J=8.0 Hz, 2H), 7.44 (d, J=8.4 Hz, 2H), 7.37 (t, J=5.9 Hz, 1H), 7.32-7.27 (m, 2H), 7.16 (s, 1H), 4.12-4.05 (m, 4H), 2.63 (d, J=8.0 Hz, 2H), 2.42 (s, 3H), 2.39 (s, 3H), 1.89-1.79 (m, 2H), 1.39 (s, 9H).

### Step 6—Tert-butyl N-[[4-(4-methylthiazol-5-yl)-2-(3-piperazin-1-ylpropyl)phenyl]methyl]carbamate

To a solution of 3-[2-[(tert-butoxycarbonylamino)methyl]-5-(4-methylthiazol-5-yl)phenyl]propyl 4-methylbenzenesulfonate (460 mg, 890 μmol) and piperazine (766 mg, 8.90 mmol) in DMF (5 mL) was added Cs₂CO₃ (1.45 g, 4.45 mmol). The mixture was then stirred at 50° C. for 16 hrs. On completion, the reaction was diluted with water (60 mL) and extracted with EtOAc (2×50 mL). The combined organic layer was dried over Na₂SO₄, filtered and concentrated in vacuo to give a residue. The residue was purified by prep-HPLC (column: Phenomenex Luna C18 200*40 mm*10 um; mobile phase: [water (TFA)-ACN]; B %: 7%-37%, 10 min) to give the title compound (380 mg, 99% yield) as a white solid. LC-MS (ESI⁺) m/z 431.5 (M+H)⁺: ¹H NMR (400 MHz, DMSO-d₆) δ 9.27 (d, J=8.8 Hz, 1H), 8.99 (s, 1H), 7.44 (s, 1H), 7.31 (d, J=14.0 Hz, 2H), 5.75 (s, 1H), 4.20 (d, J=4.8 Hz, 2H), 3.38 (s, 6H), 3.19 (s, 2H), 2.75-2.65 (m, 2H), 2.61-2.53 (m, 2H), 2.46 (s, 3H), 1.92 (s, 2H), 1.40 (s, 9H).

### Synthesis of Tert-butyl N-[[2-[3-[4-[(3R)-3-amino-4-phenylsulfanyl-butyl]piperazin-1-yl]propyl]-4-(4-methylthiazol-5-yl)phenyl]methyl]carbamate (Intermediate TR)

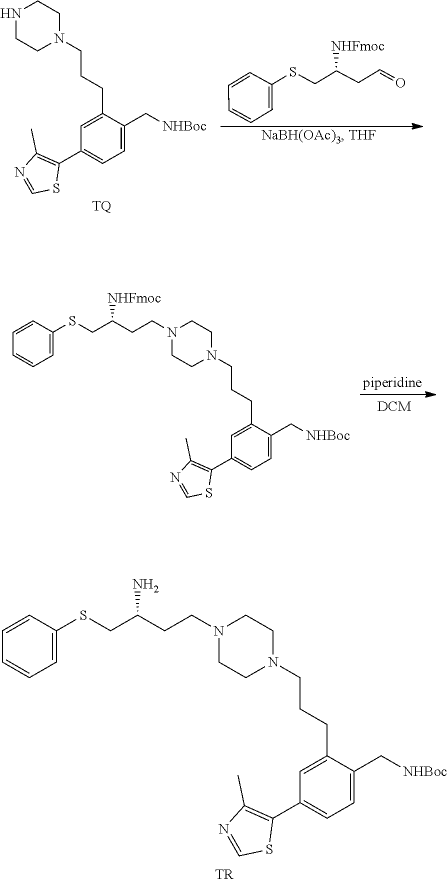

### Step 1—9H-fluoren-9-ylmethyl N-[(1R)-3-[4-[3-[2-[(tert-butoxycarbonylamino)methyl]-5-(4-methylthiazol-5-yl)phenyl]propyl]piperazin-1-yl]-1-(phenylsulfanylmethyl)propyl]carbamate

To a solution of 9H-fluoren-9-ylmethyl N-[(1R)-3-oxo-1-(phenylsulfanylmethyl)propyl]carbamate (203 mg, 487 μmol, synthesized via Steps 1-3 of Intermediate KK) and tert-butyl N-[[4-(4-methylthiazol-5-yl)-2-(3-piperazin-1-ylpropyl)phenyl]methyl]carbamate (350 mg, 812 μmol, Intermediate TQ) in THF (5 mL) was added NaBH(OAc)₃ (206 mg, 975 μmol). Then the mixture was stirred at 25° C. for 1 hr. On completion, the residue was diluted with water (30 mL) and extracted with EtOAc (2×30 mL). The combined organic layer was dried over Na₂SO₄, filtered and concentrated in vacuo to give the title compound (670 mg, 99% yield) as a white solid. LC-MS (ESI⁺) m/z 832.7 (M+H)⁺: ¹H NMR (400 MHz, DMSO-d₆) δ 11.97 (s, 1H), 9.59-9.18 (m, 1H), 8.99 (s, 1H), 7.89 (d, J=7.6 Hz, 2H), 7.72-7.66 (m, 2H), 7.41 (t, J=7.4 Hz, 3H), 7.35-7.29 (m, 8H), 7.17 (s, 1H), 5.75 (s, 2H), 4.38-4.28 (m, 2H), 4.25-4.17 (m, 3H), 3.64 (s, 1H), 3.53-3.38 (m, 2H), 3.04 (d, J=5.6 Hz, 3H), 2.67 (s, 3H), 2.46 (s, 3H), 2.30-2.15 (m, 2H), 1.91 (s, 5H), 1.83-1.68 (m, 2H), 1.65-1.53 (m, 1H), 1.40 (s, 9H).

### Step 2—Tert-butyl N-[[2-[3-[4-[(3R)-3-amino-4-phenylsulfanyl-butyl]piperazin-1-yl]propyl]-4-(4-methylthiazol-5-yl)phenyl]methyl]carbamate

To a solution of 9H-fluoren-9-ylmethyl N-[(1R)-3-[4-[3-[2-[(tert-butoxycarbonylamino)methyl]-5-(4-methylthiazol-5-yl)phenyl]propyl]piperazin-1-yl]-1-(phenylsulfanylmethyl)propyl]carbamate (580 mg, 697 μmol) in DCM (5 mL) was added piperidine (296 mg, 3.49 mmol). The mixture was then stirred at 25° C. for 7 hrs. On completion, the reaction mixture was concentrated in vacuo. The residue was purified by column chromatography (SiO₂, DCM:MeOH=50:1 to 10:1, DCM:MeOH=10:1, P1:Rf=0.2) to give the title compound (350 mg, 82% yield) as a white solid. LC-MS (ESI⁺) m/z 610.8 (M+H)⁺.

### Synthesis of N-[4-[[(1R)-3-[4-[3-[2-(aminomethyl)-5-(4-methylthiazol-5-yl)phenyl]propyl]piperazin-1-yl]-1-(phenylsulfanylmethyl)propyl]amino]-3-(trifluoromethylsulfonyl)phenyl)sulfonyl-4-[4-[[2-(4-chlorophenyl)-5,5-dimethylcyclohexen-1-yl]methyl]piperazin-1-yl]benzamide (Intermediate TS)

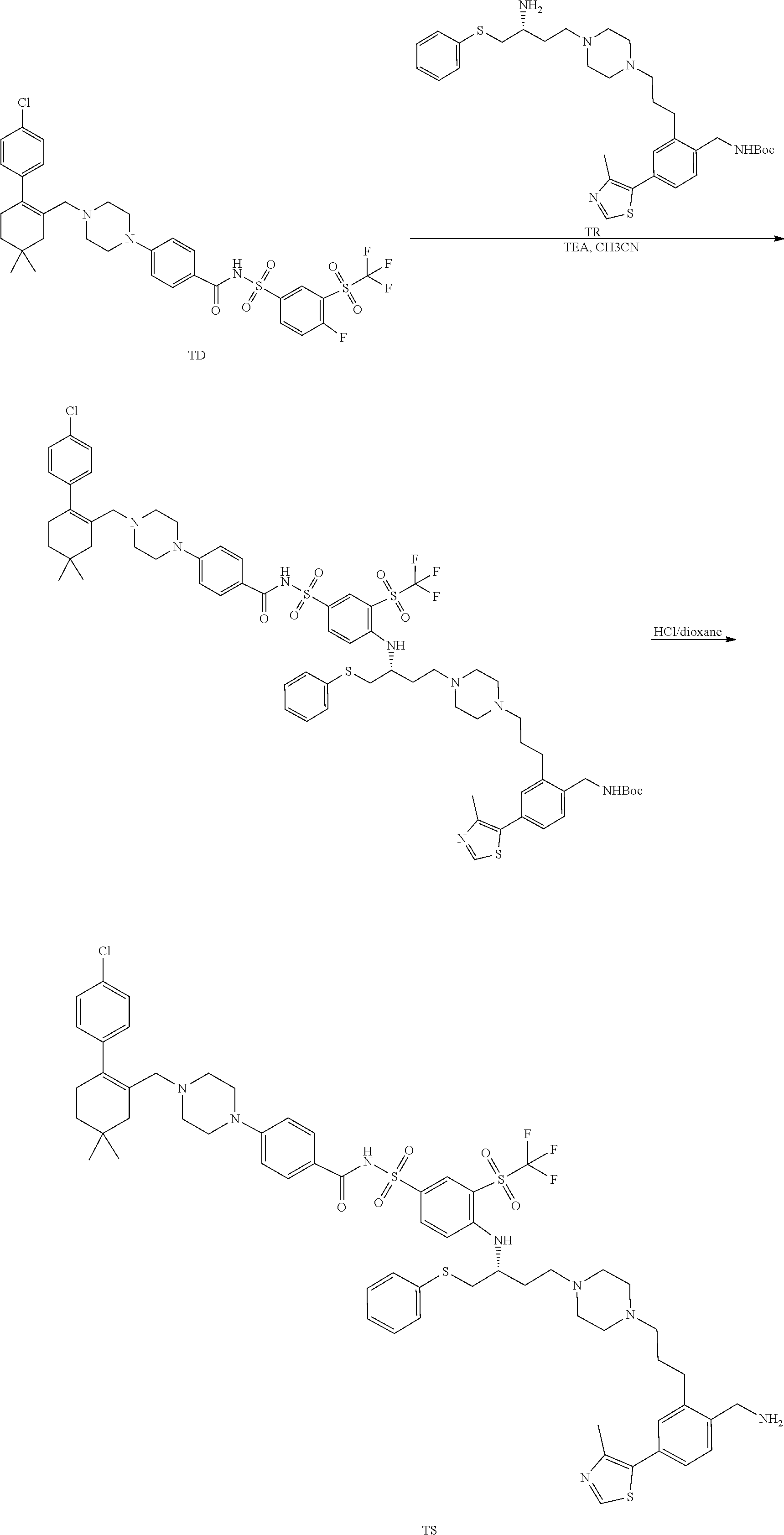

### Step 1—Tert-butyl N-[[2-[3-[4-[(3R)-3-[4-[[4-[4-[[2-(4-chlorophenyl)-5,5-dimethyl-cyclohexen-1-yl]methyl]piperazin-1-yl]benzoyl]sulfamoyl]-2-(trifluoromethylsulfonyl)anilino]-4-phenylsulfanyl-butyl]piperazin-1-yl]propyl]-4-(4-methylthiazol-5-yl)phenyl]methyl]carbamate

To a solution of tert-butyl N-[[2-[3-[4-[(3R)-3-amino-4-phenylsulfanyl-butyl]piperazin-1-yl]propyl]-4-(4-methylthiazol-5-yl)phenyl]methyl]carbamate (64.4 mg, 105 μmol, Intermediate TR) and 4-[4-[[2-(4-chlorophenyl)-5,5-dimethyl-cyclohexen-1-yl]methyl]piperazin-1-yl]-N-[4-fluoro-3 (trifluoromethylsulfonyl)phenyl]sulfonyl-benzamide (70 mg, 96.1 μmol, Intermediate TD) in CH₃CN (2 mL) was added TEA (29.1 mg, 288 μmol). The mixture was then stirred at 60° C. for 16 hrs. On completion, the reaction mixture was concentrated in vacuo. The residue was diluted with water (30 mL) and extracted with EtOAc (15 mL×2). The combined organic layer was dried over Na₂SO₄, filtered and concentrated in vacuo. The residue was purified by prep-HPLC (column: Phenomenex luna C18 150*25 mm*10 um; mobile phase: [water (FA)-ACN]; B %: 32%-62%, 15 min) to give a residue to give the title compound (33 mg, 26% yield) as a white solid. LC-MS (ESI⁺) m/z 1319.5 (M+H)+.

### Step 2—N-[4-[[(1R)-3-[4-[3-[2-(aminomethyl)-5-(4-methylthiazol-5-yl)phenyl]propyl]piperazin-1-yl]-1-(phenylsulfanylmethyl)propyl]amino]-3(trifluoromethylsulfonyl)phenyl]sulfonyl-4-[4-[[2-(4-chlorophenyl)-5,5-dimethylcyclohexen-1-yl]methyl]piperazin-1-yl]benzamide

A solution of tert-butyl N-[[2-[3-[4-[(3R)-3-[4-[[4-[4-[[2-(4-chlorophenyl)-5,5-dimethyl-cyclohexen-1-yl]methyl]piperazin-1-yl]benzoyl]sulfamoyl]-2-(trifluoromethylsulfonyl)anilino]-4-phenylsulfanyl-butyl]piperazin-1-yl]propyl]-4-(4-methylthiazol-5-yl)phenyl]methyl]carbamate (32 mg, 24.2 μmol) in HCl/dioxane (1 mL) was stirred at 25° C. for 1 h. On completion, the reaction mixture was concentrated in vacuo give the title compound (30 mg, 98% yield) as a yellow solid. LC-MS (ESI⁺) m/z 1219.2 (M+H)⁺.

### Synthesis of (R)-4-((4-morpholino-1-(phenylthio)butan-2-yl)amino)-3-((trifluoromethyl)sulfonyl)benzenesulfonamide (Intermediate TT)

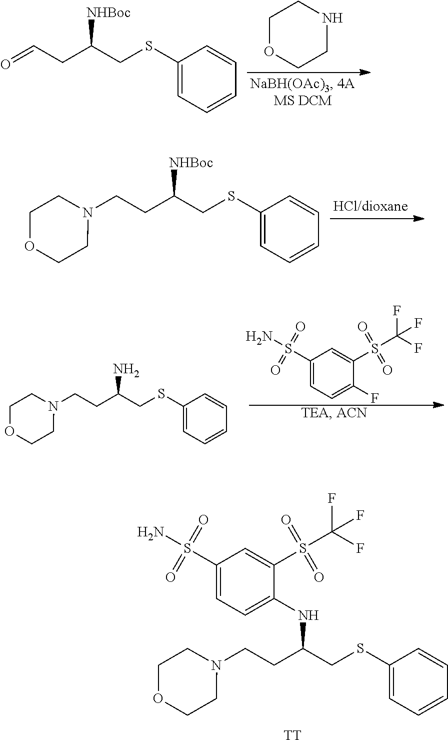

### Step 1—(R)-tert-butyl (4-morpholino-1-(phenylthio)butan-2-yl)carbamate

To a solution of (R)-tert-butyl (4-oxo-1-(phenylthio)butan-2-yl)carbamate (4.70 g, 15.9 mmol, synthesized via Step 1 of Intermediate KX), morpholine (1.39 g, 15.9 mmol, 1.40 mL) and 4 Å molecular sieves (5.00 g) in DCM (100 mL) was added NaBH(OAc)₃ (3.37 g, 15.9 mmol). The mixture was then stirred at 25° C. for 12 h. On completion, the mixture was filtered and diluted with water (40 mL), and extracted with DCM (50 mL×3). The combined organic layers were washed with brine (20 mL×3), dried over Na₂SO₄ and evaporated. The residue was purified by column chromatography (SiO₂, Petroleum ether/Ethyl acetate=1/1 to 0/1) to afford the title compound (5.40 g, 93% yield) as a white solid. ¹H NMR (400 MHz, CDCl₃) δ=7.40 (d, J=7.6 Hz, 2H), 7.31-7.27 (m, 2H), 7.19 (d, J=7.2 Hz, 1H), 5.64 (br d, J=0.8 Hz, 1H), 3.87 (br s, 1H), 3.71 (br t, J=4.4 Hz, 4H), 3.27-3.19 (m, 1H), 3.02 (br dd, J=6.8, 12.8 Hz, 1H), 2.50-2.41 (m, 6H), 1.89 (br dd, J=6.0, 12.4 Hz, 1H), 1.71 (br d, J=6.0 Hz, 1H), 1.43 (s, 9H).

### Step 2—(R)-4-morpholino-1-(phenylthio)butan-2-amine

A solution of (R)-tert-butyl (4-morpholino-1-(phenylthio)butan-2-yl)carbamate (5.40 g, 14.7 mmol) in HCl/dioxane (4 M, 54.0 mL) was stirred at 25° C. for 1 h. On completion, the mixture was evaporated to afford the title compound (4.50 g, HCl salt) as a white solid.

### Step 3—(R)-4-((4-morpholino-1-(phenylthio)butan-2-yl)amino)-3-((trifluoromethyl)sulfonyl)benzenesulfonamide

To a solution of (R)-4-morpholino-1-(phenylthio)butan-2-amine (4.50 g, 14.9 mmol, HCl salt) and 4-fluoro-3-((trifluoromethyl)sulfonyl)benzenesulfonamide (4.57 g, 14.9 mmol, CAS #1027345-08-9) in ACN (45 mL) was added TEA (1.50 g, 14.9 mmol, 2.07 mL). The mixture was stirred at 20° C. for 12 h. On completion, the reaction mixture was diluted with water (40 mL), and extracted with EtOAc (60 mL×3). The combined organic layers were washed with brine (20 mL×3), dried over Na₂SO₄ and evaporated. The residue was purified by column chromatography (SiO₂, Petroleum ether/Ethyl acetate=0/1 to Dichloromethane:Methanol=10/1) to afford the title compound (5.56 g, 68% yield) as a white solid. ¹H NMR (400 MHz, CDCl₃) δ=8.26 (d, J=2.0 Hz, 1H), 7.82 (br d, J=9.2 Hz, 1H), 7.42-7.38 (m, 2H), 7.36-7.29 (m, 3H), 7.05 (br d, J=8.4 Hz, 1H), 6.65 (br d, J=8.8 Hz, 1H), 4.85 (br s, 2H), 3.95 (br dd, J=6.8, 8.4 Hz, 1H), 3.68 (br s, 4H), 3.13-3.03 (m, 2H), 2.54-2.28 (m, 6H), 2.20-2.09 (m, 1H), 1.71 (br s, 1H).

### Synthesis of (R)—N-((4-((4-morpholino-1-(phenylthio)butan-2-yl)amino)-3-((trifluoromethyl)sulfonyl)phenyl)sulfonyl)-4-(piperazin-1-yl)benzamide (Intermediate TU)

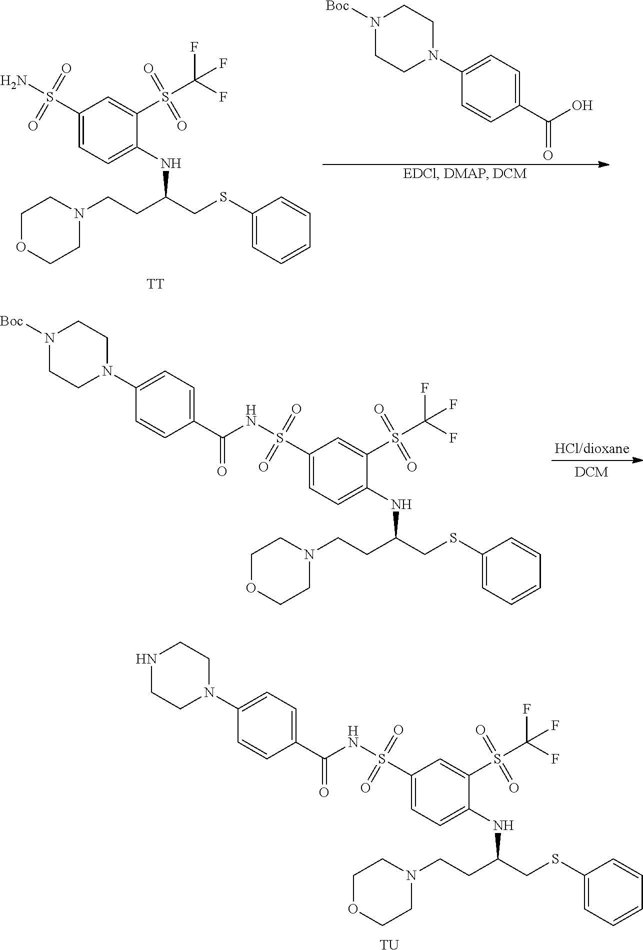

### Step 1—(R)-tert-butyl 4-(4-(((4-((4-morpholino-1-(phenylthio)butan-2-yl)amino)-3-((trifluoromethyl)sulfonyl)phenyl)sulfonyl)carbamoyl)phenyl)piperazine-1-carboxylate

To a mixture of 4-(4-(tert-butoxycarbonyl)piperazin-1-yl)benzoic acid (415 mg, 1.35 mmol, CAS #162046-66-4), (R)-4-((4-morpholino-1-(phenylthio)butan-2-yl)amino)-3-((trifluoromethyl)sulfonyl)benzenesulfonamide (500 mg, 903 μmol, Intermediate TT) and DMAP (166 mg, 1.35 mmol) in DCM (10 mL) was added EDCI (260 mg, 1.35 mmol). The mixture was stirred at 25° C. for 12 h. On completion, the reaction mixture was diluted with water (15 mL), and extracted with DCM (20 mL×3). The combined organic layers were washed with brine (10 mL×3), dried over Na₂SO₄ and evaporated. The residue was purified by column chromatography (SiO₂, Dichloromethane:Methanol=20/1 to 10/1) to afford the title compound (500 mg, 66% yield) as a white solid. LC-MS (ESI⁺) m/z 842.3 (M+H)⁺.

### Step 2—(R)—N-((4-((4-morpholino-1-(phenylthio)butan-2-yl)amino)-3-((trifluoromethyl)sulfonyl)phenyl)sulfonyl)-4-(piperazin-1-yl)benzamide

To a mixture of (R)-tert-butyl 4-(4-(((4-((4-morpholino-1-(phenylthio)butan-2-yl)amino)-3-((trifluoromethyl)sulfonyl)phenyl)sulfonyl)carbamoyl)phenyl)piperazine-1-carboxylate (500 mg, 594 μmol) in DCM (2 mL) was added HCl/dioxane (4 M, 5.00 mL). The mixture was stirred at 25° C. for 0.5 h. On completion, the reaction mixture was evaporated to afford the title compound (500 mg, HCl salt) as a white solid. LC-MS (ESI⁺) m/z 742.3 (M+H)⁺.

### Synthesis of Ethyl 4′-chloro-6-formyl-4-methyl-2,3,4,5-tetrahydro-[1,1′-biphenyl]-4-carboxylate (Intermediate TV)

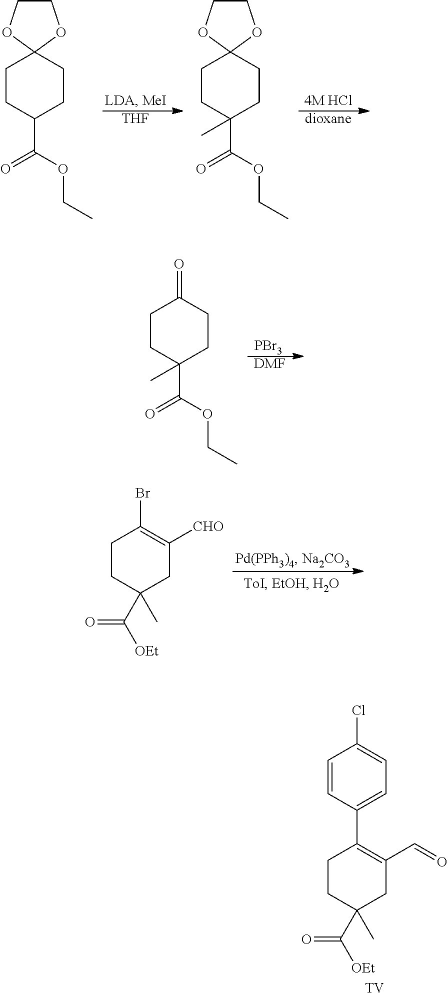

### Step 1—Ethyl 8-methyl-1,4-dioxaspiro[4.5]decane-8-carboxylate

To a solution of ethyl 1,4-dioxaspiro[4.5]decane-8-carboxylate (10.0 g, 46.7 mmol, CAS #1489-97-0) in THF (150 mL) was added LDA (2 M, 37.3 mL) dropwise at −78° C. under N₂ and the mixture was stirred for 30 min. Then a solution of CH₃I (19.9 g, 140 mmol, 8.72 mL) in THF (50 mL) was added to at −78° C. The mixture was warmed to 20° C. and stirred for 12 h. On completion, the reaction mixture was quenched by addition of sat. NH₄Cl (100 mL), and extracted with EtOAc (150 mL×3). The combined organic layers were washed with brine (100 mL×3), dried over Na₂SO₄ and evaporated. The residue was purified by flash silica gel chromatography (Petroleum ether/Ethyl acetate=0/1 to 9/1) to afford title compound (10.5 g, 99% yield) as a colorless oil. ¹H NMR (400 MHz, CDCl₃) δ=4.14 (q, J=7.2 Hz, 2H), 3.93 (s, 4H), 2.13 (br d, J=12.8 Hz, 2H), 1.67-1.57 (m, 4H), 1.51 (br dd, J=4.4, 12.8 Hz, 2H), 1.25 (t, J=7.2 Hz, 3H), 1.18 (s, 3H).

### Step 2—Ethyl 1-methyl-4-oxocyclohexanecarboxylate

To a solution of ethyl 8-methyl-1,4-dioxaspiro[4.5]decane-8-carboxylate (10.5 g, 46.0 mmol) in dioxane (100 mL) was added HCl (4 M, 100 mL). The mixture was stirred at 25° C. for 12 h. On completion, the reaction mixture was quenched by addition of sat. NH₄Cl (50 mL), and extracted with EtOAc (50 mL×3). The combined organic layers were washed with brine (30 mL×3), dried over Na₂SO₄ and evaporated. The residue was purified by column chromatography (SiO₂, Petroleum ether/Ethyl acetate=1/0 to 20/1) to afford title compound (8.40 g, 99% yield) as a colorless oil.

### Step 3—Ethyl 4-bromo-3-formyl-1-methylcyclohex-3-enecarboxylate

To a solution of ethyl 1-methyl-4-oxocyclohexanecarboxylate (5.50 g, 29.9 mmol) in PBr₃ (32.3 g, 119 mmol) was added DMF (6.55 g, 89.6 mmol, 6.89 mL) at 0° C. under N₂ and stirred for 20 min. Then PBr₃ (16.2 g, 59.7 mmol) was added and the mixture was stirred for 10 min. The reaction mixture was heated to 60° C. and stirred for 12 h. On completion, the reaction mixture was diluted with ice water (200 mL), and extracted with DCM (200 mL×3). The combined organic layers were washed with brine (30 mL×3), dried over Na₂SO₄ and evaporated. The residue was purified by column chromatography (SiO₂, Petroleum ether/Ethyl acetate=0/1 to 15/1) to afford title compound (2.59 g, 32% yield) as a yellow oil. ¹H NMR (400 MHz, CDCl₃) δ=10.01 (s, 1H), 4.14 (q, J=7.2 Hz, 2H), 2.91-2.71 (m, 3H), 2.18-2.09 (m, 2H), 1.76-1.66 (m, 1H), 1.29-1.20 (m, 6H).

### Step 4—Ethyl 4′-chloro-6-formyl-4-methyl-2,3,4,5-tetrahydro-[1,1′-biphenyl]-4-carboxylate

To a solution of ethyl 4-bromo-3-formyl-1-methylcyclohex-3-enecarboxylate (2.59 g, 9.41 mmol), (4-chlorophenyl)boronic acid (1.77 g, 11.3 mmol) and Na₂CO₃ (2 M, 9.41 mL) in toluene (13 mL) and EtOH (7 mL) was added Pd(PPh₃)₄ (544 mg, 471 μmol) under N₂. Then the mixture was heated to 90° C. for 7 h. On completion, the reaction mixture was diluted with water (50 mL), and extracted with EtOAc (80 mL×3). The combined organic layers were washed with brine (10 mL×3), dried over Na₂SO₄ and evaporated. The crude product was purified by column chromatography (SiO₂, Petroleum ether/Ethyl acetate=0/1 to 20/1) to afford title compound (2.88 g, 99% yield) as a yellow oil. ¹H NMR (400 MHz, CDCl₃) δ=9.49 (s, 1H), 7.40-7.32 (m, 2H), 7.15 (d, J=8.4 Hz, 2H), 4.19-4.11 (m, 2H), 2.96 (dd, J=1.6, 17.6 Hz, 1H), 2.68-2.53 (m, 2H), 2.23-2.14 (m, 2H), 1.75-1.67 (m, 1H), 1.31 (s, 3H), 1.25 (t, J=7.2 Hz, 3H).

### Synthesis of 4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carboxylic Acid (Intermediate TW)

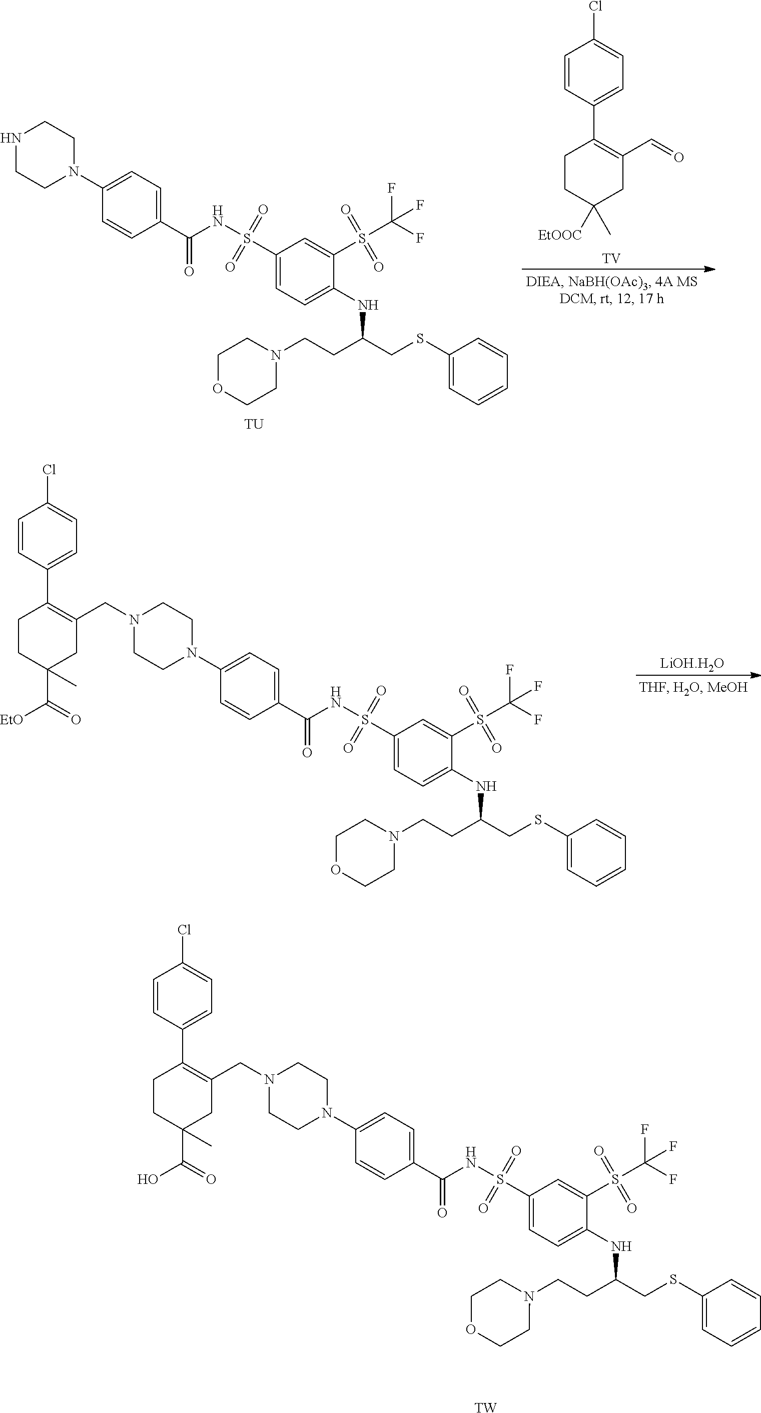

### Step 1—Ethyl 4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carboxylate

A solution of (R)—N-((4-((4-morpholino-1-(phenylthio)butan-2-yl)amino)-3-((trifluoromethyl)sulfonyl)phenyl)sulfonyl)-4-(piperazin-1-yl)benzamide (254 mg, 326 μmol, HCl salt, Intermediate TU) in DCM (2 mL) was added DIEA (42.1 mg, 326 μmol, 56.8 uL) and stirred at 25° C. for 10 min. Then ethyl 4′-chloro-6-formyl-4-methyl-2,3,4,5-tetrahydro-[1,1′-biphenyl]-4-carboxy late (100 mg, 326 μmol, Intermediate TV), 4A MS (100 mg) and NaBH(OAc)₃ (69.1 mg, 326 μmol) was added and the mixture was stirred at 25° C. for 12 h. On completion, the reaction mixture was quenched by addition of sat. NH₄Cl (15 mL) and filtered, then extracted with DCM (20 mL×3). The combined organic layers were washed with brine (3 mL×3), dried over Na₂SO₄ and evaporated. The residue was purified by column chromatography (SiO₂, Petroleum ether/Ethyl acetate=1/1 to 1/8) to afford the title compound (280 mg, 83% yield) as a yellow solid. ¹H NMR (400 MHz, CDCl₃) δ=8.38 (br s, 1H), 8.22-8.06 (m, 1H), 7.70-7.60 (m, 2H), 7.43-7.29 (m, 9H), 7.12-7.06 (m, 1H), 6.96 (br d, J=8.4 Hz, 1H), 6.80 (br d, J=8.4 Hz, 1H), 6.62 (br d, J=9.2 Hz, 1H), 4.23-4.13 (m, 2H), 3.96-3.90 (m, 1H), 3.72-3.66 (m, 4H), 3.39-3.31 (m, 3H), 3.16-3.05 (m, 4H), 2.95-2.78 (m, 4H), 2.49-2.31 (m, 11H), 2.16-2.09 (m, 3H), 1.49-1.44 (m, 4H), 1.29 (br s, 3H).

### Step 2—4′-Chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carboxylic Acid

A solution of ethyl 4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carboxylate (100 mg, 96.8 μmol) and LiOH·H₂O (32.5 mg, 775 μmol) in THF (1 mL). MeOH (0.2 mL) and H₂O (0.2 mL) was stirred at 25° C. for 12 h and 40° C. for 2 h. On completion, the reaction mixture was evaporated and diluted with water (5 mL), then the pH was adjusted to 4˜3 by addition of 2M HCl. Then the mixture was extracted with EtOAc (10 mL×3). The combined organic layers were washed with brine (2 mL×3), dried over Na₂SO₄ and evaporated to afford the title compound (100 mg) as a yellow solid. LC-MS (ESI+) m/z 1004.3 (M+H)⁺.

### Synthesis of 8-(4-(Tert-butoxycarbonyl)piperazin-1-yl)-8-oxooctanoic Acid (Intermediate TX)

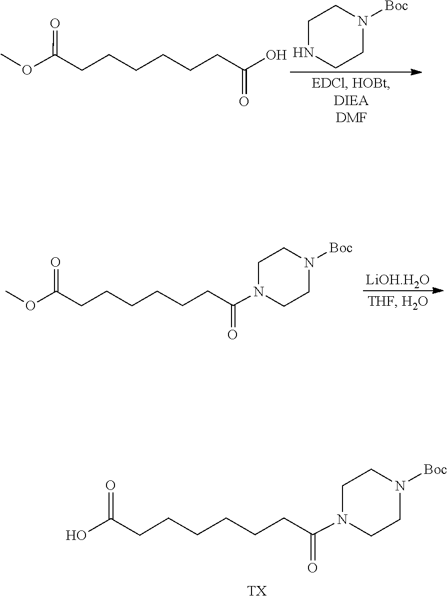

### Step 1—Tert-butyl 4-(8-methoxy-8-oxooctanoyl)piperazine-1-carboxylate

To a solution of 8-methoxy-8-oxooctanoic acid (5.00 g, 26.6 mmol, 4.76 mL, CAS #3946-32-5), tert-butyl piperazine-1-carboxylate (7.10 g, 31.9 mmol. HCl salt, CAS #143238-38-4) and DIEA (17.2 g, 133 mmol, 23.1 mL) in DMF (50 mL) was added EDCI (7.64 g, 39.9 mmol) and HOBt (5.38 g, 39.9 mmol). Then the mixture was stirred at 25° C. for 12 h. On completion, the reaction mixture was diluted with water (50 mL), and extracted with EtOAc (80 mL×3). The combined organic layers were washed with brine (40 mL×5), dried over Na₂SO₄ and evaporated. The residue was purified by column chromatography (SiO₂, Petroleum ether/Ethyl acetate=3/1 to 2/1) to afford the title compound (9.00 g, 95% yield) as a colorless oil. ¹H NMR (400 MHz, CDCl₃) δ=3.67 (s, 3H), 3.59 (br d, J=4.8 Hz, 2H), 3.47-3.38 (m, 6H), 2.35-2.29 (m, 4H), 1.64 (br t, J=6.8 Hz, 4H), 1.48 (s, 9H), 1.36 (br t, J=3.6 Hz, 4H).

### Step 2—8-(4-(Tert-butoxycarbonyl)piperazin-1-yl)-8-oxooctanoic Acid

To a solution of tert-butyl 4-(8-methoxy-8-oxooctanoyl)piperazine-1-carboxylate (5.00 g, 14.0 mmol) in THF (50 mL) and H₂O (10 mL) was added LiOH·H₂O (1.18 g, 28.1 mmol). Then the mixture was stirred at 25° C. for 12 h. On completion, the reaction mixture was diluted with water (5 mL) and the pH was adjusted to 3-4 by addition of 2M HCl, then extracted with EtOAc (25 mL×3). The combined organic layers were washed with brine (10 mL×3), dried over Na₂SO₄ and evaporated to afford the title compound (4.60 g, 96% yield) as a white solid. ¹H NMR (400 MHz, CDCl₃) δ=3.59 (br s, 2H), 3.44 (br s, 6H), 2.35 (dt, J=4.0, 7.6 Hz, 4H), 1.64 (br d, J=7.2 Hz, 4H), 1.48 (s, 9H), 1.41-1.33 (m, 4H).

### Synthesis of (2S,4R)-1-(S)-3,3-dimethyl-2-(8-oxo-8-(piperazin-1-yl) octanamido)butanoyl)-4-hydroxy-N—((S)-1-(4-(4-methylthiazol-5-yl)phenyl)ethyl)pyrrolidine-2-carboxamide (Intermediate TY)

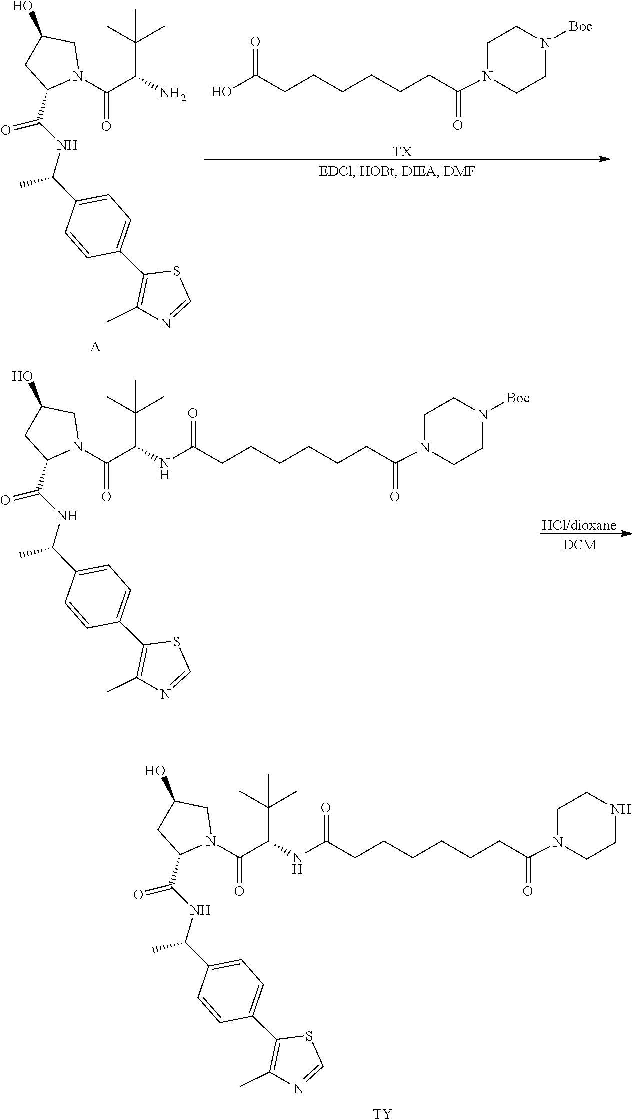

### Step 1—Tert-butyl 4-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)piperazine-1-carboxylate

To a solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (1.00 g, 2.08 mmol, HCl salt, Intermediate A), 8-(4-(tert-butoxycarbonyl)piperazin-1-yl)-8-oxooctanoic acid (854 mg, 2.49 mmol, Intermediate TX) and DIEA (1.34 g, 10.4 mmol, 1.81 mL) in DMF (20 mL) was added EDCI (598 mg, 3.12 mmol) and HOBt (421 mg, 3.12 mmol). The mixture was then stirred at 25° C. for 12 h. On completion, the reaction mixture was diluted with water (15 mL), and extracted with EtOAc (20 mL×3). The combined organic layers were washed with brine (10 mL×5), dried over Na₂SO₄ and evaporated. The crude product was purified by reversed-phase HPLC (ACN/0.1% NH₃·H₂O=70%) to afford the title compound (400 mg, 19% yield) as a brown oil. LC-MS (ESI⁺) m/z 769.6 (M+H)⁺.

### Step 2—(2S,4R)-1-((S)-3,3-dimethyl-2-(8-oxo-8-(piperazin-1-yl) octanamido)butanoyl)-4-hydroxy-N—((S)-1-(4-(4-methylthiazol-5-yl)phenyl)ethyl)pyrrolidine-2-carboxamide

To a solution of tert-butyl 4-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)piperazine-1-carboxylate (400 mg, 401 μmol) in DCM (2 mL) was added HCl/dioxane (4 M, 4.00 mL). The mixture was then stirred at 25° C. for 1 h. On completion, the reaction mixture evaporated to afford the title compound (300 mg, HCl salt) as a brown solid. LC-MS (ESI⁺) m/z 669.6 (M+H)⁺.

### Synthesis of (2S,4R)-4-hydroxy-N-(2-hydroxy-4-(4-methylthiazol-5-yl)benzyl)pyrrolidine-2-carboxamide (Intermediate TZ)

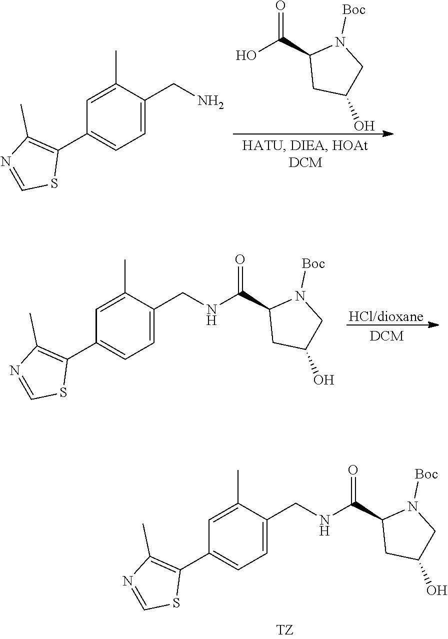 [SMILES: Cc1cc(-c2scnc2C)ccc1CN ; CC(C)(C)OC(=O)N1C[C@H](O)C[C@H]1C(=O)O ; Cc1cc(-c2scnc2C)ccc1CNC(=O)[C@@H]1C[C@@H](O)CN1C(=O)OC(C)(C)C ; Cc1cc(-c2scnc2C)ccc1CNC(=O)[C@@H]1C[C@@H](O)CN1C(=O)OC(C)(C)C]

### Step 1—(2S,4R)-tert-butyl 4-hydroxy-2-((2-hydroxy-4-(4-methylthiazol-5-yl)benzyl)carbamoyl) pyrrolidine-1-carboxylate

A solution of (2S,4R)-1-(tert-butoxycarbonyl)-4-hydroxypyrrolidine-2-carboxylic acid (5.85 g, 25.3 mmol, CAS #13726-69-7) and 2-(aminomethyl)-5-(4-methylthiazol-5-yl)phenol (5 g, 19.4 mmol, HCl, CAS #1448190-11-1) in DCM (180 mL) was cooled to −10° C. Then, HOAt (3.45 g, 25.3 mmol), DIEA (12.5 g, 97.3 mmol, 16.9 mL) and HATU (8.15 g, 21.4 mmol) was added and the mixture was stirred at −10° C. for 1 h. On completion, the reaction mixture was quenched by addition of H₂O (200 mL) at 20° C., and extracted with DCM (200 mL×3). The combined organic layers were washed with DCM (200 mL×2), dried over Na₂SO₄, filtered and concentrated under reduced pressure to give a residue. The residue was purified by column chromatography (SiO₂, Petroleum ether/Ethyl acetate=5/1 to 3/1) to give the title compound (7.5 g, 89% yield) as yellow solid. ¹H NMR (400 MHz, DMSO-d₆) δ=9.97-9.77 (m, 1H), 8.96 (s, 1H), 8.51-8.30 (m, 1H), 7.27-7.17 (m, 1H), 6.93-6.82 (m, 2H), 4.25 (br s, 2H), 4.19 (s, 1H), 3.62 (dtd, J=4.0, 6.4, 13.2 Hz, 2H), 3.39 (br d, J=4.0 Hz, 1H), 3.28 (br d, J=17.2 Hz, 1H), 2.44 (s, 3H), 2.17-2.06 (m, 1H), 1.93-1.84 (m, 1H), 1.24 (d, J=3.6 Hz, 9H).

### Step 2—(2S,4R)-4-hydroxy-N-(2-hydroxy-4-(4-methylthiazol-5-yl)benzyl)pyrrolidine-2-carboxamide

A solution of (2S,4R)-tert-butyl 4-hydroxy-2-((2-hydroxy-4-(4-methylthiazol-5-yl)benzyl)carbamoyl)pyrrolidine-1-carboxylate (7.5 g, 17.3 mmol) in DCM (15 mL) was added HCl/dioxane (4 M, 15 mL) and the mixture was stirred at 25° C. for 12 h. On completion, concentrated in vacuo to give the title compound (6 g) as yellow solid. LC-MS (ESI+) m/z 334.0 (M+H)⁺.

### Synthesis of (2S,4R)-4-hydroxy-1-(3-methyl-2-(3-methylisoxazol-5-yl)butanoyl)-N-(4-(4-methylthiazol-5-yl)-2-(2-oxoethoxy)benzyl)pyrrolidine-2-carboxamide (Intermediate UA)

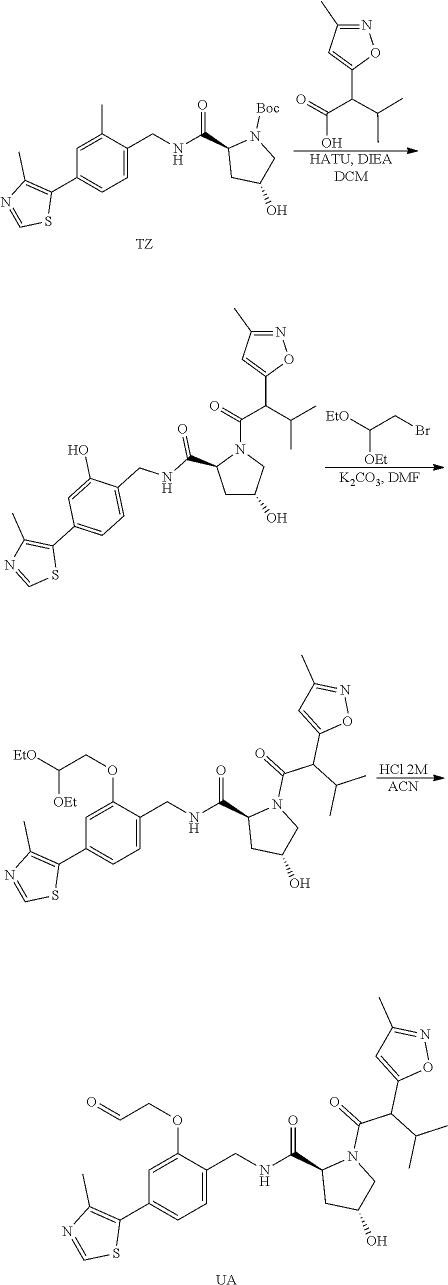

### Step 1—(2S,4R)-4-hydroxy-N-(2-hydroxy-4-(4-methylthiazol-5-yl)benzyl)-1-(3-methyl-2-(3-methylisoxazol-5-yl)butanoyl)pyrrolidine-2-carboxamide

To a solution of 3-methyl-2-(3-methylisoxazol-5-yl)butanoic acid (1 g, 5.46 mmol, synthesized via Steps 1-3 of Intermediate SY) in DCM (20 mL) was added HATU (2.28 g, 6.00 mmol) and DIEA (3.53 g, 27.2 mmol, 4.75 mL), and the mixture was stirred at 0° C. for 0.5 h. Then (2S,4R)-4-hydroxy-N-(2-hydroxy-4-(4-methylthiazol-5-yl)benzyl)pyrrolidine-2-carboxamide (2.18 g, 6.55 mmol, Intermediate TZ) was added and the mixture was stirred at 25° C. for 0.5 h. On completion, the reaction mixture was quenched by addition of H₂O (20 mL) at 20° C., and extracted with DCM (20 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=5/1 to 0/1) to give the title compound (1.2 g, 44% yield) as yellow gum. LC-MS (ESI+) m/z 499.4 (M+H)⁺.

### Step 2—(2S,4R)—N-(2-(2,2-diethoxyethoxy)-4-(4-methylthiazol-5-yl)benzyl)-4-hydroxy-1-(3-methyl-2-(3-methylisoxazol-5-yl)butanoyl)pyrrolidine-2-carboxamide

To a solution of (2S,4R)-4-hydroxy-N-(2-hydroxy-4-(4-methylthiazol-5-yl)benzyl)-1-(3-methyl-2-(3-methylisoxazol-5-yl)butanoyl)pyrrolidine-2-carboxamide (300 mg, 601 μmol) in DMF (3 mL) was added K₂CO₃ (249 mg, 1.81 mmol) and 2-bromo-1,1-diethoxy-ethane (355 mg, 1.81 mmol, 271 uL), then the mixture was stirred at 70° C. for 12 h. On completion, the reaction mixture was quenched by addition of H₂O (20 mL) at 20° C., and extracted with EtOAc (20 mL×3). The combined organic layers were washed with brine (20 mL×2), dried over Na₂SO₄, filtered and concentrated under reduced pressure to give a residue. The residue was purified by column chromatography (SiO₂, Petroleum ether/Ethyl acetate=5/1 to 0/1) to give the title compound (180 mg, 49% yield) as brown solid. LC-MS (ESI+) m/z 637.4 (M+Na)⁺.

### Step 3—(2S,4R)-4-hydroxy-1-(3-methyl-2-(3-methylisoxazol-5-yl)butanoyl)-N-(4-(4-methylthiazol-5-yl)-2-(2-oxoethoxy)benzyl)pyrrolidine-2-carboxamide

To a solution of (2S,4R)—N-(2-(2,2-diethoxyethoxy)-4-(4-methylthiazol-5-yl)benzyl)-4-hydroxy-1-(3-methyl-2-(3-methylisoxazol-5-yl)butanoyl)pyrrolidine-2-carboxamide (180 mg, 292 μmol) in ACN (2 mL) was added HCl (2 M, 146.40 uL), then the mixture was stirred at 50° C. for 12 h. On completion, the mixture was concentrated in vacuo to give the title compound (130 mg, 82% yield) as yellow solid. LC-MS (ESI+) m/z 539.4 (M−H)⁺.

### Synthesis of (S)-4-(6-(2-(2-cyclopropylphenyl)pyrrolidin-1-yl)-2-azaspiro[3.3]heptan-2-yl)benzoic Acid (Intermediate UB)

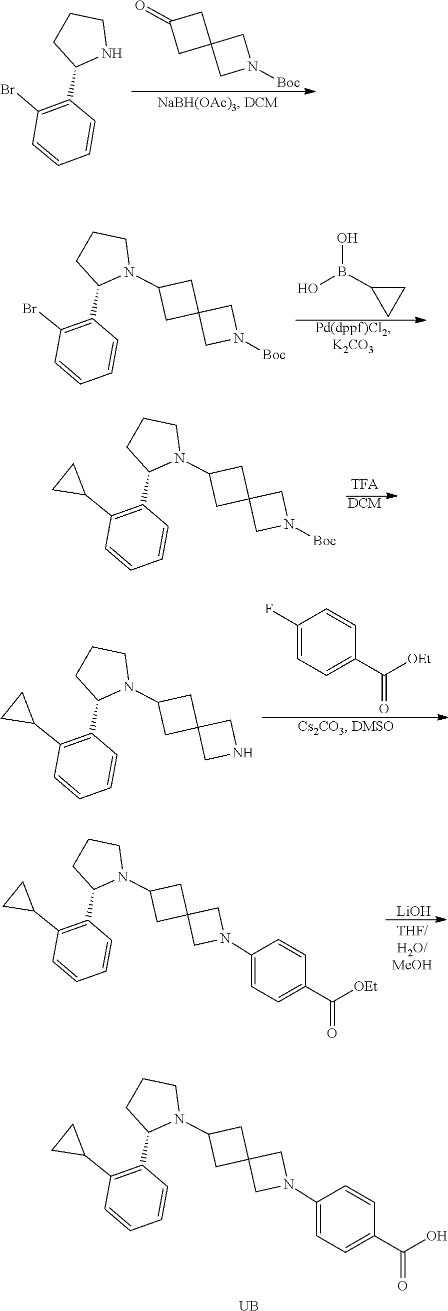

### Step 1—(S)-tert-butyl 6-(2-(2-bromophenyl)pyrrolidin-1-yl)-2-azaspiro[3.3]heptane-2-carboxylate

To a solution of (2S)-2-(2-bromophenyl)pyrrolidine (2.00 g, 8.85 mmol, CAS #1217680-27-7), tert-butyl 6-oxo-2-azaspiro[3.3]heptane-2-carboxylate (1.87 g, 8.85 mmol, CAS #1181816-12-5) in DCM (20 mL) was added NaBH(OAc)₃ (3.77 g, 17.8 mmol). The mixture was then stirred at 25° C. for 2 h. On completion, the reaction was poured into water (15 mL) and extracted with DCM (20 mL×3). The combined organic phase was washed with brine (10 mL×2), dried over sodium sulfate, filtered and concentrated to give a residue. The residue was purified by column chromatography (SiO₂, Petroleum ether/Ethyl acetate=0/1 to Dichloromethane:Methanol=20:1) to give the title compound (3.90 g) as a brown oil. LC-MS (ESI⁺) m/z 421.1 (M+H)⁺.

### Step 2—(S)-tert-butyl 6-(2-(2-cyclopropylphenyl)pyrrolidin-1-yl)-2-azaspiro[3.3]heptane-2-carboxylate

To a solution of (S)-tert-butyl 6-(2-(2-bromophenyl)pyrrolidin-1-yl)-2-azaspiro[3.3]heptane-2-carboxylate (3.90 g, 9.26 mmol) and cyclopropylboronic acid (3.98 g, 46.3 mmol, CAS #411235-57-9) in H₂O (5 mL) and dioxane (45 mL) was added K₂CO₃ (5.12 g, 37.0 mmol) and Pd(dppf)Cl₂·CH₂Cl₂ (755.8 mg, 925.6 μmol) under N₂. The mixture was then stirred at 100° C. for 4 h. On completion, the reaction was poured into water (50 mL) and extracted with EtOAc (100 mL×3). The combined organic phase is washed with brine (50 mL×2), dried over sodium sulfate, filtered and concentrated to give a residue. The crude product was purified by reversed-phase HPLC (ACN/0.1% FA=40%) to give the title compound (900 mg, 25% yield) as brown oil. LC-MS (ESI⁺) m/z 383.2 (M+H)⁺; ¹H NMR (400 MHz, DMSO-d₆) δ=7.48 (dd, J=1.6, 7.6 Hz, 1H), 7.12 (dtd, J=1.2, 7.2, 19.2 Hz, 2H), 6.96-6.90 (m, 1H), 3.94 (t, J=8.0 Hz, 1H), 3.78-3.56 (m, 5H), 3.12-3.06 (m, 1H), 2.34 (q, J=8.5 Hz, 1H), 2.26-2.16 (m, 1H), 2.08-2.02 (m, 3H), 1.82-1.72 (m, 4H), 1.56-1.48 (m, 1H), 1.32 (s, 9H), 0.94-0.84 (m, 2H), 0.68-0.62 (m, 1H), 0.58-0.48 (m, 1H).

### Step 3—(S)-6-(2-(2-cyclopropylphenyl)pyrrolidin-1-yl)-2-azaspiro[3.3]heptane

To a solution of tert-butyl 6-[(2S)-2-(2-cyclopropylphenyl)pyrrolidin-1-yl]-2-azaspiro[3.3]heptane-2-carboxylate (900 mg, 2.35 mmol) in DCM (9 mL) was added TFA (4.62 g, 40.5 mmol, 3 mL). The mixture was then stirred at 25° C. for 2 h. On completion, the reaction mixture was concentrated under reduced pressure to give the title compound (800 mg) as a brown oil. LC-MS (ESI⁺) m/z 283.1 (M+H)⁺.

### Step 4—(S)-6-(2-(2-cyclopropylphenyl)pyrrolidin-1-yl)-2-azaspiro[3.3]heptane

To a solution of 6-[(2S)-2-(2-cyclopropylphenyl)pyrrolidin-1-yl]-2-azaspiro[3.3]heptane (780 mg, 2.76 mmol) and ethyl 4-fluorobenzoate (557 mg, 3.31 mmol, 488 uL, CAS #451-46-7) in DMSO (8 mL) was added Cs₂CO₃ (4.50 g, 13.8 mmol). The mixture was then stirred at 80° C. for 12 h. On completion, the reaction was poured into water (8 mL) and extracted with EtOAc (15 mL×3). The combined organic phase is washed with brine (10 mL×2), dried over sodium sulfate, filtered and concentrated to give a residue. The residue was purified by column chromatography (SiO₂, Petroleum ether/Ethyl acetate=5/1) to give the title compound (560 mg, 47% yield) as white solid. LC-MS (ESI⁺) m/z 431.3 (M+H)⁺.

### Step 5—(S)-4-(6-(2-(2-cyclopropylphenyl)pyrrolidin-1-yl)-2-azaspiro[3.3]heptan-2-yl)benzoic Acid

To a solution of (S)-6-(2-(2-cyclopropylphenyl)pyrrolidin-1-yl)-2-azaspiro[3.3]heptane (470 mg, 1.09 mmol) in THF (4 mL). H₂O (4 mL), and MeOH (4 mL) was added LiOH·H₂O (229 mg, 5.46 mmol). The mixture was stirred at 25° C. for 2 h. On completion, the pH of the reaction mixture was adjusted to 6˜5 by addition 2 M HCl, then the mixture was extracted with EtOAc (10 ml×5). The organic phase was separated, dried over sodium sulfate, filtered and concentrated under reduced pressure to give the title compound (710 mg) as a yellow solid. LC-MS (ESI⁺) m/z 403.2 (M+H)⁺.

### Synthesis of 4-(6-((S)-2-(2-cyclopropylphenyl)pyrrolidin-1-yl)-2-azaspiro[3.3]heptan-2-yl)-N-((4-(((R)-1-(phenylthio)-4-(piperazin-1-yl)butan-2-yl)amino)-3-((trifluoromethyl)sulfonyl)phenyl)sulfonyl)benzamide (Intermediate UC)

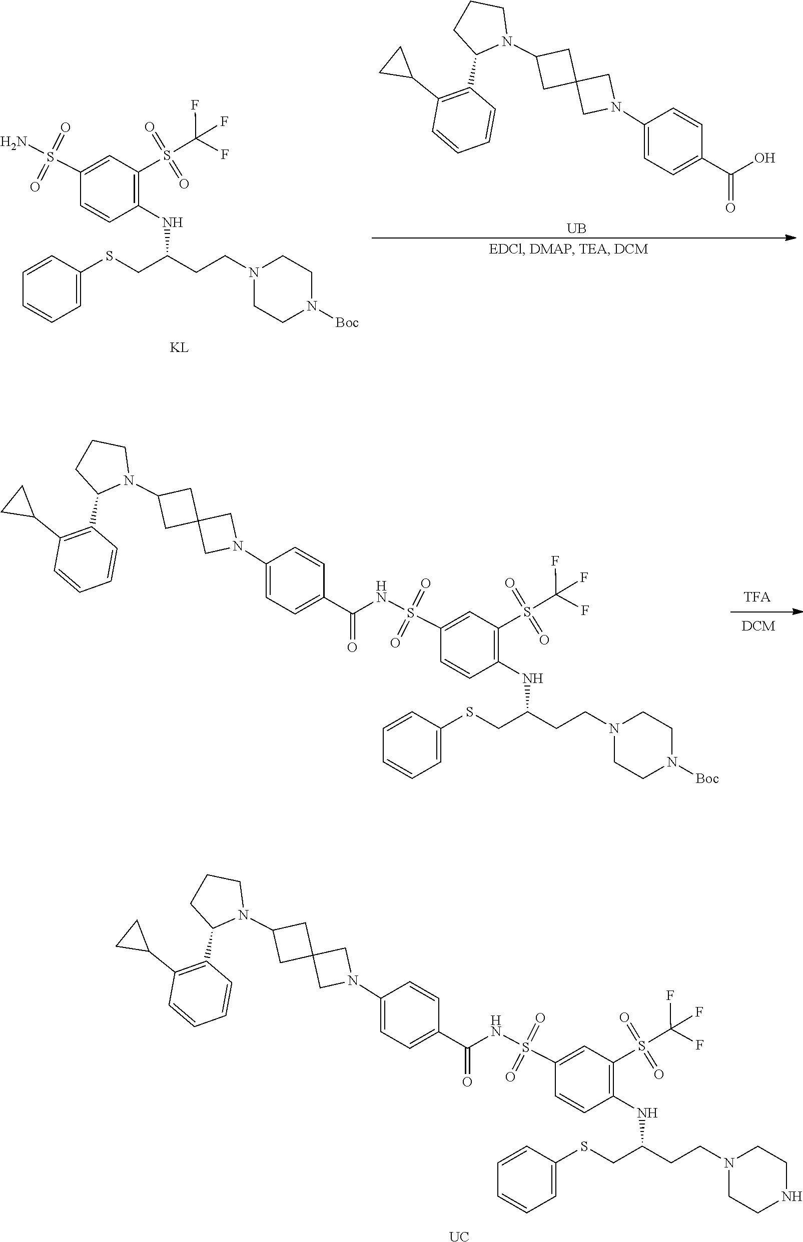 [SMILES: CC(C)(C)OC(=O)N1CCN(CC[C@H](CSc2ccccc2)Nc2ccc(S(N)(=O)=O)cc2S(=O)(=O)C(F)(F)F)CC1 ; O=C(O)c1ccc(N2CC3(CC(N4CCC[C@H]4c4ccccc4C4CC4)C3)C2)cc1 ; "UB" ; CC(C)(C)OC(=O)N1CCN(CC[C@H](CSc2ccccc2)Nc2ccc(S(=O)(=O)NC(=O)c3ccc(N4CC5(CC(N6CCC[C@H]6c6ccccc6C6CC6)C5)C4)cc3)cc2S(=O)(=O)C(F)(F)F)CC1 ; O=C(NS(=O)(=O)c1ccc(N[C@H](CCN2CCNCC2)CSc2ccccc2)c(S(=O)(=O)C(F)(F)F)c1)c1ccc(N2CC3(CC(N4CCC[C@H]4c4ccccc4C4CC4)C3)C2)cc1 ; "UC"]

### Step 1—Tert-butyl 4-[(3R)-3-[4-[[4-[6-[(2S)-2-(2-cyclopropylphenyl)pyrrolidin-1-yl]-2-azaspiro[3.3]heptan-2-yl]benzoyl]sulfamoyl]-2-(trifluoromethylsulfonyl)anilino]-4-phenylsulfanyl-butyl]piperazine-1-carboxylate

To a solution of (S)-4-(6-(2-(2-cyclopropylphenyl)pyrrolidin-1-yl)-2-azaspiro[3.3]heptan-2-yl)benzoic acid (350 mg, 869 μmol, Intermediate UB), (R)-tert-butyl 4-(3-((2-((2-fluoropropan-2-yl)sulfonyl)-4-sulfamoylphenyl)amino)-4-(phenylthio)butyl)piperazine-1-carboxylate (681.1 mg, 1.04 mmol, Intermediate KL) and TEA (202 mg, 2.00 mmol, 278 uL) in DCM (4 mL) was added EDCI (667 mg, 3.48 mmol) and DMAP (127 mg, 1.04 mmol). The mixture was then stirred at 25° C. for 12 h. On completion, the reaction mixture was filtered and concentrated under reduced pressure to give a residue. The crude product was purified by reversed-phase HPLC (column: Welch Ultimate C18 150*25 mm*5 um; mobile phase: [water (TFA)-ACN]; B %: 32%-62%, 10 min) to give title compound (300 mg, 33% yield) as a white solid. LC-MS (ESI⁺) m/z 519.6 (M+H)⁺.

### Step 2—4-(6-((S)-2-(2-cyclopropylphenyl)pyrrolidin-1-yl)-2-azaspiro[3.3]heptan-2-yl)-N-((4-(((R)-1-(phenylthio)-4-(piperazin-1-yl)butan-2-yl)amino)-3-((trifluoromethyl)sulfonyl)phenyl)sulfonyl)benzamide

To a solution of tert-butyl 4-[(3R)-3-[4-[[4-[6-[(2S)-2-(2-cyclopropylphenyl)pyrrolidin-1-yl]-2-azaspiro[3.3]heptan-2-yl]benzoyl]sulfamoyl]-2-(trifluoromethylsulfonyl)anilino]-4-phenylsulfanyl-butyl]piperazine-1-carboxylate (200 mg, 193 μmol) in DCM (2 mL) was added TFA (308 mg, 2.70 mmol, 0.2 mL). The mixture was then stirred at 25° C. for 2 h. On completion, the reaction mixture was filtered and concentrated under reduced pressure to give the title compound (200 mg) as a yellow solid. LC-MS (ESI+) m/z 937.6 (M+H)⁺.

### Synthesis of (2S,4R)-1-((S)-2-(1-fluorocyclopropanecarboxamido)-3,3-dimethylbutanoyl)-4-hydroxy-N-(4-(4-methylthiazol-5-yl)-2-(piperidin-4-yloxy)benzyl)pyrrolidine-2-carboxamide (Intermediate UD)

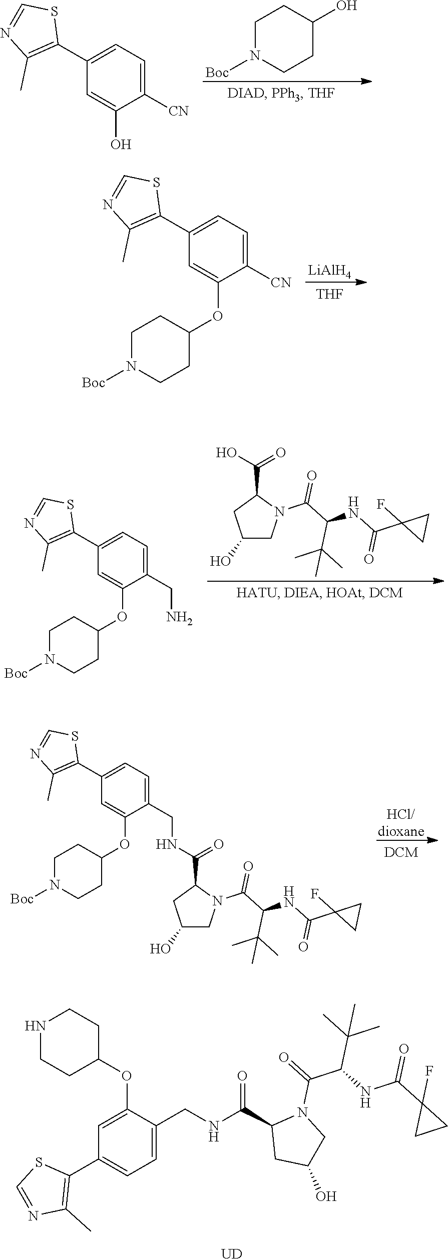

### Step 2—Tert-butyl 4-(2-cyano-5-(4-methylthiazol-5-yl)phenoxy)piperidine-1-carboxylate

To a solution of 2-hydroxy-4-(4-methylthiazol-5-yl)benzonitrile (5 g, 23.1 mmol, CAS #1448190-10-0), tert-butyl 4-hydroxypiperidine-1-carboxylate (5.12 g, 25.4 mmol) and PPh₃ (9.10 g, 34.7 mmol) in THF (50 mL) was added dropwise over 30 min a solution of DIAD (5.61 g, 27.7 mmol) in THF (50 mL) with ice bath cooling to maintain the reaction temperature at 0° C. The resulting solution was then stirred at 25° C. for 12 h. On completion, the mixture was concentrated under reduced pressure to give a residue. The residue was purified by column chromatography (SiO₂, Petroleum ether/Ethyl acetate=20/1-5/1) to give the title compound (6 g, 65% yield) as a yellow solid. ¹H NMR (400 MHz, CDCl₃-d) δ=8.78 (s, 1H), 7.63 (d, J=8.0 Hz, 1H), 7.10 (d, J=8.0 Hz, 1H), 7.01 (s, 1H), 4.72-4.65 (m, 1H), 3.71-3.61 (m. 2H), 3.58-3.47 (m, 2H), 2.57 (s, 3H), 1.98-1.87 (m, 4H), 1.48 (s, 9H).

### Step 2—Tert-butyl 4-(2-(aminomethyl)-5-(4-methylthiazol-5-yl)phenoxy)piperidine-1-carboxylate

To a solution of tert-butyl 4-(2-cyano-5-(4-methylthiazol-5-yl)phenoxy)piperidine-1-carboxylate (800 mg, 2.00 mmol) in THF (8 mL) was added LiAlH₄ (152 mg, 4.00 mmol). The mixture was then stirred at 0° C. for 2 h. On completion, the mixture was quenched by H₂O (0.152 mL×6) at 0° C. The mixture was then filtered and concentrated in vacuo. The crude product was purified by reversed-phase HPLC (0.1% FA condition) to give the title compound (200 mg, 25% yield) as a yellow oil. LC-MS (ESI⁺) m/z 404.2 (M+H)⁺; ¹H NMR (400 MHz, DMSO-d₆) δ=9.02 (s, 1H), 8.24 (s, 1H), 7.50-7.42 (m, 1H), 7.17 (s, 1H), 7.11-7.05 (m, 1H), 3.96 (s, 2H), 3.64-3.61 (m, 2H), 3.29 (br d, J=3.2 Hz, 2H), 2.74-2.69 (m, 1H), 2.46 (s, 3H), 1.95-1.87 (m, 2H), 1.72-1.59 (m, 2H), 1.42-1.39 (m, 9H).

### Step 3—Tert-butyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)piperidine-1-carboxylate

To a solution of tert-butyl 4-(2-(aminomethyl)-5-(4-methylthiazol-5-yl)phenoxy)piperidine-1-carboxylate (200 mg, 495 μmol) and (2S,4R)-1-((S)-2-(1-fluorocyclopropanecarboxamido)-3,3-dimethylbutanoyl)-4-hydroxypyrrolidine-2-carboxylic acid (163 mg, 495 μmol, CAS #2316837-29-1) in DCM (3 mL) was added DIEA (192 mg, 1.49 mmol, 258 uL) at −10° C. Then HATU (188 mg, 495 μmol) and HOAt (74.2 mg, 545 μmol) was added at −10° C. in batches and the mixture was stirred at −10° C. for 1 h. On completion, the mixture was concentrated under reduced pressure to give a residue. The residue was purified by column chromatography (SiO₂, Petroleum ether:Ethyl acetate=5:1 to Petroleum ether:Ethyl acetate=0:1) to give the title compound (150 mg, 42% yield) as a white solid. LC-MS (ESI⁺) m/z 716.3 (M+H)⁺.

### Step 4—(2S,4R)-1-((S)-2-(1-fluorocyclopropanecarboxamido)-3,3-dimethylbutanoyl)-4-hydroxy-N-(4-(4-methylthiazol-5-yl)-2-(piperidin-4-yloxy)benzyl)pyrrolidine-2-carboxamide

To a solution of tert-butyl 4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)piperidine-1-carboxylate (150 mg, 209 μmol in DCM (2 mL) was added HCl/dioxane (4 M. 52.3 uL). The mixture was then stirred at 25° C. for 1 h. On completion, the mixture was concentrated in vacuo to give the title compound (120 mg) as a white solid. LC-MS (ESI⁺) m/z 616.2 (M+H)⁺.

### Synthesis of (2S,4R)—N-(2-((1-((R)-3-amino-4-(phenylthio)butyl)piperidin-4-yl)oxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (Intermediate UE)

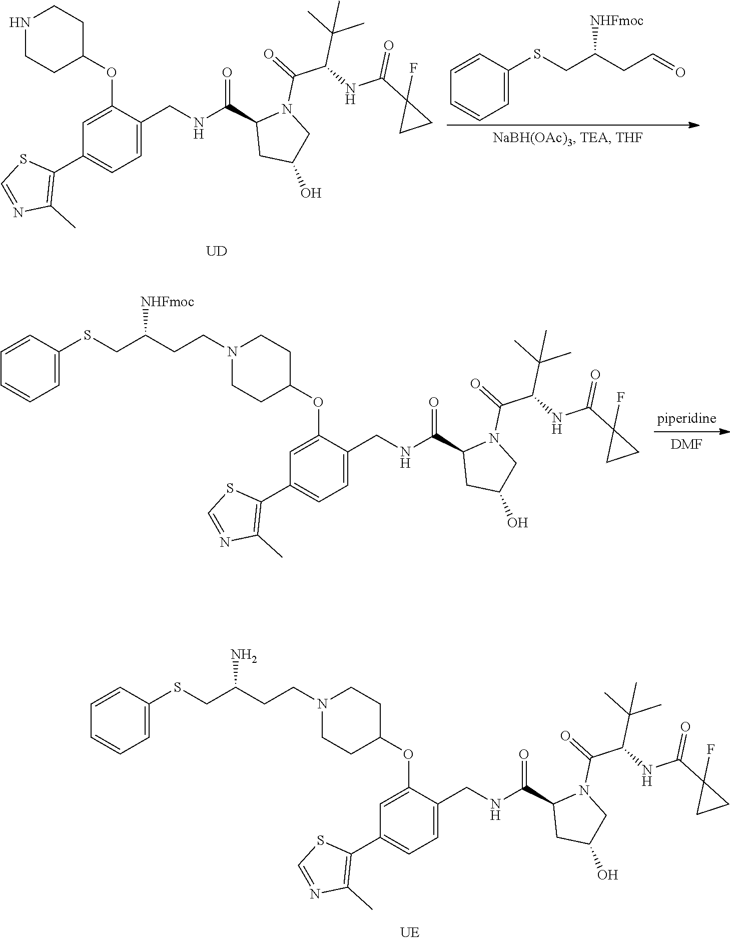

### Step 1—(9H-fluoren-9-yl)methyl ((R)-4-(4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)piperidin-1-yl)-1-(phenylthio)butan-2-yl)carbamate

To a solution of (2S,4R)-1-((S)-2-(1-fluorocyclopropanecarboxamido)-3,3-dimethylbutanoyl)-4-hydroxy-N-(4-(4-methylthiazol-5-yl)-2-(piperidin-4-yloxy)benzyl)pyrrolidine-2-carboxamide (110 mg, 178 μmol, Intermediate UD), (R)-(9H-fluoren-9-yl)methyl (4-oxo-1-(phenylthio)butan-2-yl)carbamate (74.6 mg, 178 μmol, synthesized via Steps 1-3 of Intermediate KK) in THF (1 mL) was added NaBH(OAc)₃ (75.7 mg, 357 μmol). The mixture was then stirred at 25° C. for 2 h. On completion, the mixture was concentrated in vacuo. The residue was purified by column chromatography (SiO₂, Petroleum ether:Ethyl acetate=5:1 to Dichloromethane:Methanol=10:1) to give the title compound (80 mg, 44% yield) as a white solid. LC-MS (ESI⁺) m/z 1017.6 (M+H)⁺.

### Step 2—(2S,4R)—N-(2-((1-((R)-3-amino-4-(phenylthio)butyl)piperidin-4-yl)oxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide

To a solution of (9H-fluoren-9-yl)methyl ((R)-4-(4-(2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)piperidin-1-yl)-1-(phenylthio)butan-2-yl)carbamate (80 mg, 78.6 μmol) in DMF (0.9 mL) was added piperidine (258 mg, 3.04 mmol, 0.3 mL). The mixture was then stirred at 25° C. for 2 h. On completion, the mixture was filtered. The filtrate was purified by reversed-phase HPLC (0.1% FA condition) to give the title compound (50 mg, 80% yield) as a yellow solid. LC-MS (ESI⁺) m/z 795.5 (M+H)⁺.

### Synthesis of (2S,4R)-1-((S)-2-(1-fluorocyclopropanecarboxamido)-3,3-dimethylbutanoyl)-4-hydroxy-N-(4-(4-methylthiazol-5-yl)-2-(piperidin-4-ylmethoxy)benzyl)pyrrolidine-2-carboxamide (Intermediate UF)

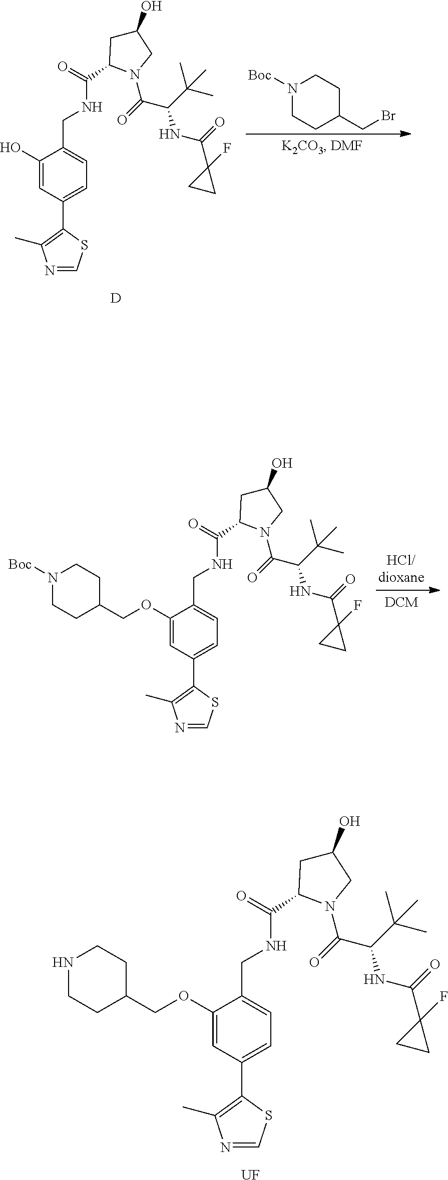

### Step 1—Tert-butyl 4-((2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)methyl)piperidine-1-carboxylate

To a solution of (2S,4R)-1-((S)-2-(1-fluorocyclopropanecarboxamido)-3,3-dimethylbutanoyl)-4-hydroxy-N-(2-hydroxy-4-(4-methylthiazol-5-yl)benzyl)pyrrolidine-2-carboxamide (800 mg, 1.50 mmol, Intermediate D) and K₂CO₃ (623 mg, 4.51 mmol) in DMF (8 mL) was added tert-butyl 4-(bromomethyl)piperidine-1-carboxylate (836 mg, 3.00 mmol, CAS #158407-04-6). The mixture was then stirred at 80° C. for 12 h. On completion, the reaction mixture was quenched by addition of H₂O (15 mL) and extracted with EtOAc (20 mL×3). The combined organic layers were dried over Na₂SO₄, filtered and concentrated under reduced pressure to give a residue. The residue was purified by column chromatography (SiO₂, Petroleum ether/Ethyl acetate=0/1 to Ethyl acetate/MeOH=10/1) to afford the title compound (900 mg, 82% yield) as a colorless oil. ¹H NMR (400 MHz, CDCl₃) δ 8.70 (s, 1H), 8.03 (s, 1H), 7.34 (d, J=7.6 Hz, 1H), 6.97 (dd, J=1.6, 7.6 Hz, 1H), 6.86 (d, J=1.2 Hz, 1H), 4.74 (t, J=7.6 Hz, 1H), 4.59-4.50 (m, 3H), 4.40 (br dd, J=5.2, 14.8 Hz, 1H), 4.22-4.17 (m, 1H), 4.02 (brd, J=11.2 Hz, 1H), 3.87 (br d, J=6.4 Hz, 2H), 3.63 (dd, J=4.0, 11.2 Hz, 1H), 2.82-2.74 (m, 2H), 2.63-2.56 (m, 1H), 2.54 (s, 3H), 2.13-2.06 (m, 1H), 1.89-1.80 (m, 2H), 1.48 (s, 9H), 1.37-1.26 (m, 7H), 0.94 (s, 9H).

### Step 2—(2S,4R)-1-((S)-2-(1-fluorocyclopropanecarboxamido)-3,3-dimethylbutanoyl)-4-hydroxy-N-(4-(4-methylthiazol-5-yl)-2-(piperidin-4-ylmethoxy)benzyl)pyrrolidine-2-carboxamide

To a solution of tert-butyl 4-((2-((((2S,4R)-1-((2S)-2-((1-fluorocyclopropanecarbonyl)amino)-3,3-dimethyl-butanoyl)-4-hydroxy-pyrrolidine-2-carbonyl)amino)methyl)-5-(4-methylthiazol-5-yl)phenoxy)methyl)piperidine-1-carboxylate (600 mg, 822 μmol) in DCM (2 mL) was added HCl/dioxane (4 M, 6 mL). The mixture was then stirred at 20° C. for 1 h. On completion, the reaction mixture was concentrated under reduced pressure to give a residue and evaporated to afford the title compound (600 mg, HCl) as a white oil. LC-MS (ESI⁺) m/z 630.4 (M+H)⁺.

### Synthesis of (2S,4R)—N-(2-((1-((R)-3-amino-4-(phenylthio)butyl)piperidin-4-yl)methoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide (Intermediate UG)

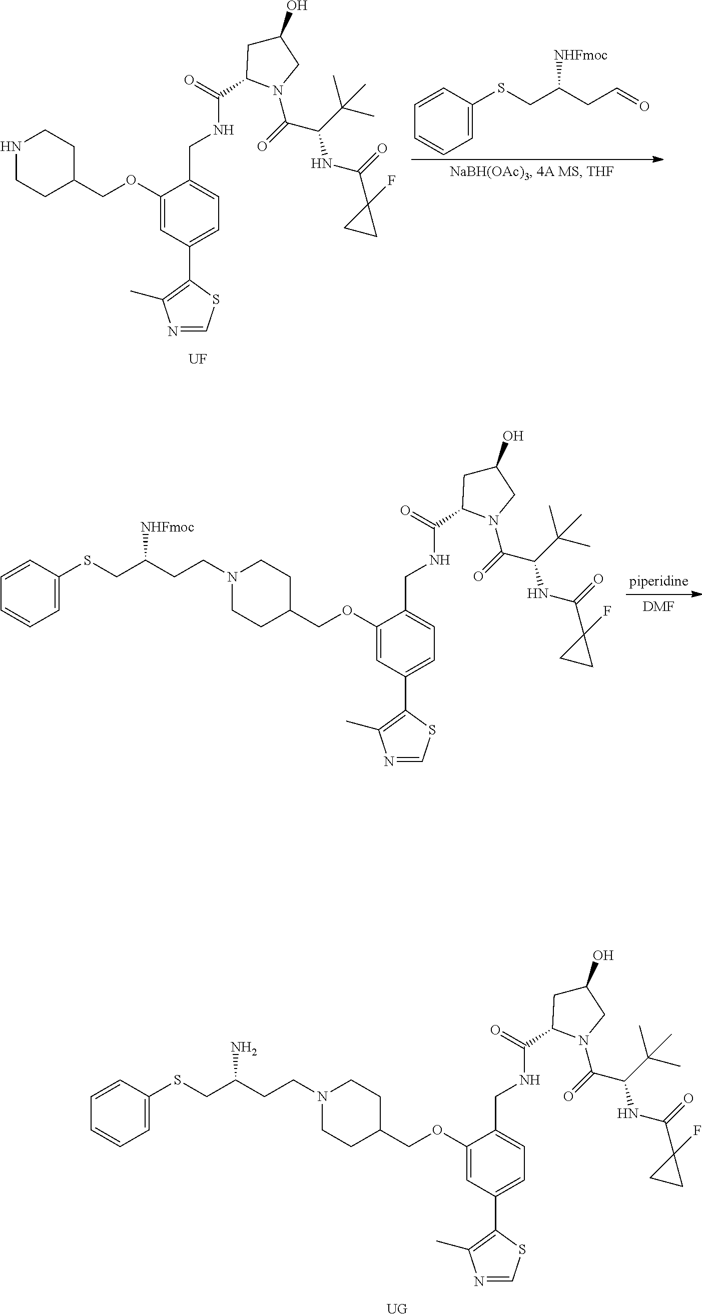

### Step 1—(9H-fluoren-9-yl)methyl ((R)-4-(4-((2-(((2S,4R)-1-((S)-2-(cyclopropanecarboxamido)-3,3-dimethylbutanoyl)-4-hydroxypyrrolidine-2-carboxamido)methyl)-5-(4-methylthiazol-5-yl)phenoxy)methyl)piperidin-1-yl)-1-(phenylthio)butan-2-yl)carbamate

To a solution of (2S,4R)-1-((S)-2-(1-fluorocyclopropanecarboxamido)-3,3-dimethylbutanoyl)-4-hydroxy-N-(4-(4-methylthiazol-5-yl)-2-(piperidin-4-ylmethoxy)benzyl)pyrrolidine-2-carboxamide (527 mg, 790 μmol, HCl, Intermediate UF) and (R)-(9H-fluoren-9-yl)methyl (4-oxo-1-(phenylthio)butan-2-yl)carbamate (300 mg, 719 μmol, synthesized via Steps 1-3 of Intermediate KK) in THF (5 mL) was added NaBH(OAc) 3 (305 mg, 1.44 mmol) and 4 Å molecular sieves (300 mg). The mixture was then stirred at 25° C. for 12 h. On completion, the mixture was quenched by addition of H₂O (15 mL) and extracted with EtOAc (25 mL×3). The combined organic layers were washed with brine (5 mL×3), dried over Na₂SO₄, filtered and concentrated under reduced pressure to give a residue. The residue was purified by column chromatography (SiO₂, Petroleum ether/Ethyl acetate=0/1 to Ethyl acetate/MeOH=10/1) to afford the title compound (230 mg, 31% yield) as a brown oil. ¹H NMR (400 MHz, CDCl₃) δ 8.70-8.68 (m, 1H), 8.70-8.68 (m, 1H), 8.69 (s, 1H), 7.75 (d, J=7.6 Hz, 2H), 7.64 (br s, 2H), 7.45-7.40 (m, 2H), 7.37 (br d, J=7.2 Hz, 2H), 7.34 (br d, J=5.6 Hz, 2H), 7.31 (br d, J=6.4 Hz, 3H), 7.21-7.16 (m, 1H), 6.98 (br d, J=6.8 Hz, 2H), 6.84-6.82 (m, 1H), 6.83 (s, 1H), 4.76-4.70 (m, 1H), 4.73 (br t, J=7.6 Hz, 1H), 4.52 (br d, J=8.8 Hz, 2H), 4.55-4.48 (m, 1H), 4.40-4.33 (m, 1H), 4.43-4.33 (m, 1H), 4.40-4.31 (m, 1H), 4.42-4.28 (m, 1H), 4.21 (br t, J=6.8 Hz, 1H), 3.98-3.80 (m, 4H), 3.65-3.61 (m, 1H), 3.49 (s, 1H), 3.34-3.27 (m, 1H), 3.04-2.98 (m, 1H), 2.53 (s, 5H), 2.23-2.06 (m, 5H), 2.04-1.97 (m, 2H), 1.37-1.29 (m, 3H), 0.93-0.93 (m, 1H), 0.91 (s, 9H).

### Step 2—(2S,4R)—N-(2-((1-((R)-3-amino-4-(phenylthio)butyl)piperidin-4-yl)methoxy)-4-(4-methylthiazol-5-yl)benzyl)-1-((S)-2-(1-fluorocyclopropane-1-carboxamido)-3,3-dimethylbutanoyl)-4-hydroxypyrrolidine-2-carboxamide

To a solution of (9H-fluoren-9-yl)methyl ((R)-4-(4-((2-(((2S,4R)-1-((S)-2-(1-fluorocyclopropanecarboxamido)-3,3-dimethylbutanoyl)-4-hydroxypyrrolidine-2-carboxamido)methyl)-5-(4-methylthiazol-5-yl)phenoxy)methyl)piperidin-1-yl)-1-(phenylthio)butan-2-yl)carbamate (230 mg, 223 μmol) in DMF (3 mL) was added piperidine (1.94 g, 22.8 mmol, 2.25 mL). The mixture was then stirred at 25° C. for 2 h. On completion, the reaction mixture was filtered and concentrated under reduced pressure. The crude product was purified by reversed-phase HPLC (ACN/0.1% NH₃·H₂O=50%) to afford the title compound (120 mg, 67% yield) as a brown oil. LC-MS (ESI⁺) m/z 809.2 (M+H)⁺.

### Synthesis of Tert-butyldimethylsilyl 2-(2-(((tert-butyldimethylsilyl)oxy)methyl)phenoxy)-4-(4-((4′-chloro-5,5-dimethyl-3,4,5,6-tetrahydro-[1,1′-biphenyl]-2-yl)methyl)piperazin-1-yl)benzoate (Intermediate UI)

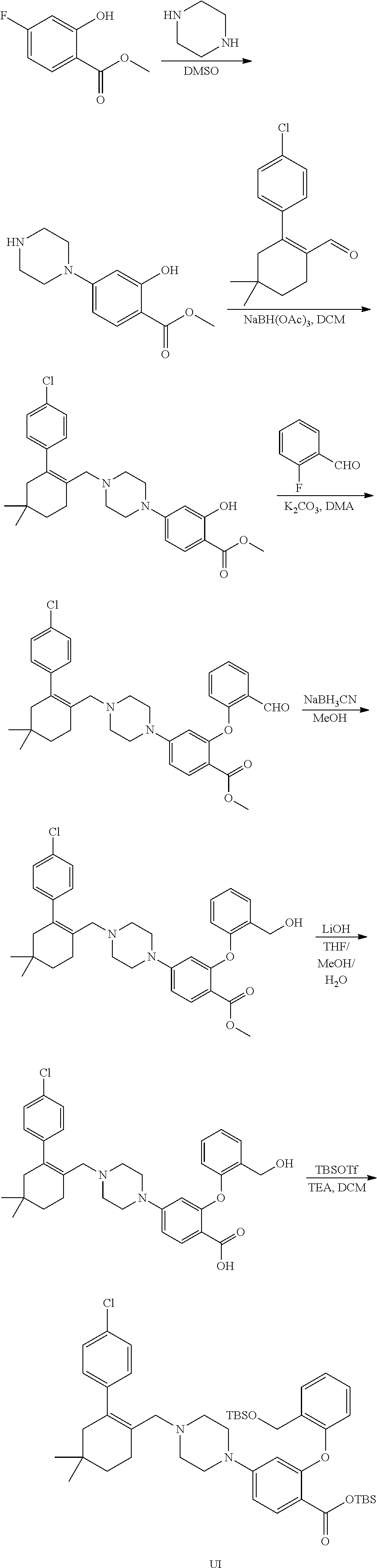

### Step 1—Methyl 2-hydroxy-4-(piperazin-1-yl)benzoate

To a solution of methyl 4-fluoro-2-hydroxy-benzoate (10 g, 58.7 mmol, CAS #392-04-1) in DMSO (150 mL) was added piperazine (15.19 g, 176 mmol, CAS #110-85-0). The mixture was then stirred at 25° C. for 48 hrs. On completion, the reaction mixture was filtered and the filtrate was concentrated in vacuo to give the crude residue. The residue was poured into water (300 mL) and extracted by ethyl acetate (3×300 mL). The combined organic layers were washed by brine (600 mL) and dried over anhydrous sodium sulfate, filtered and the filtrate was concentrated in vacuo to give the title compound (8 g) as a white solid. LC-MS (ESI+) m/z 237.0 (M+1)⁺.

### Step 2—Methyl 4-(4-((4′-chloro-5,5-dimethyl-3,4,5,6-tetrahydro-[1,1′-biphenyl]-2-yl)methyl)piperazin-1-yl)-2-hydroxybenzoate

To a solution of methyl 2-hydroxy-4-(piperazin-1-yl)benzoate (2.5 g, 10.5 mmol) and 4′-chloro-5,5-dimethyl-3,4,5,6-tetrahydro-[1,1′-biphenyl]-2-carbaldehyde (2.6 g, 10.5 mmol, CAS #1228837 May 5) in DCM (30 mL) was added NaBH(OAc)₃ (2.92 g, 13.7 mmol) at 25° C., then the reaction was stirred at 25° C. for 3 hrs. On completion, the mixture was quenched with water (20 mL) and extracted with dichloromethane (3×20 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=10/1 to 5/1) to give the title compound (4.5 g, 88% yield) as a yellow oil. LC-MS (ESI+) m/z 469.2 (M+1)⁺.

### Step 3—Methyl 4-(4-((4′-chloro-5,5-dimethyl-3,4,5,6-tetrahydro-[1,1′-biphenyl]-2-yl)methyl)piperazin-1-yl)-2-(2-formylphenoxy)benzoate

To a solution of methyl 4-(4-((4′-chloro-5,5-dimethyl-3,4,5,6-tetrahydro-[1,1′-biphenyl]-2-yl)methyl)piperazin-1-yl)-2-hydroxybenzoate (5.4 g, 11.5 mmol) and 2-fluorobenzaldehyde (1.71 g, 13.8 mmol, CAS #446-52-6) in DMA (50 mL) was added K₂CO₃ (3.18 g, 23.0 mmol) at 25° C., then the mixture was stirred at 120° C. for 12 hrs. On completion, the reaction mixture was quenched with water (30 mL) and extracted by ethyl acetate (3×20 mL). The combined organic layers were washed by brine (60 mL) and dried over anhydrous sodium sulfate, filtered and the filtrate was concentrated in vacuo to give the title compound (2 g, 28% yield) as a white solid. LC-MS (ESI+) m/z 573.2 (M+1)⁺.

### Step 4—Methyl 4-(4-((4′-chloro-5,5-dimethyl-3,4,5,6-tetrahydro-[1,1′-biphenyl]-2-yl)methyl)piperazin-1-yl)-2-(2-(hydroxymethyl)phenoxy)benzoate

To a solution of methyl 4-(4-((4′-chloro-5,5-dimethyl-3,4,5,6-tetrahydro-[1,1′-biphenyl]-2-yl)methyl)piperazin-1-yl)-2-(2-formylphenoxy)benzoate (2 g, 3.49 mmol) in MeOH (20 mL) and AcOH (0.2 mL) was added NaBH₃CN (263 mg, 4.19 mmol) at 0° C., then the mixture was stirred at 25° C. for 12 hrs. On completion, the reaction mixture was filtered and the filtrate was concentrated in vacuo to give the crude residue. The residue was purified by column chromatography (SiO₂, Petroleum ether/Ethyl acetate=3/1) to give the title compound (2 g, 93% yield) as a yellow oil. LC-MS (ESI+) m/z 575.6 (M+1)⁺.

### Step 5—4-(4-((4′-Chloro-5,5-dimethyl-3,4,5,6-tetrahydro-[1,1′-biphenyl]-2-yl)methyl)piperazin-1-yl)-2-(2-(hydroxymethyl)phenoxy)benzoic Acid

To a solution of methyl 4-(4-((4′-chloro-5,5-dimethyl-3,4,5,6-tetrahydro-[1,1′-biphenyl]-2-yl)methyl)piperazin-1-yl)-2-(2-(hydroxymethyl)phenoxy)benzoate (2 g, 3.48 mmol) in anhydrous THF (20 mL), MeOH (5 mL) and H₂O (5 mL) was added LiOH·H₂O (1.46 g, 34.7 mmol) at 25° C., then the reaction was stirred at 40° C. for 12 hrs. On completion. HCl (1N) was added to the reaction mixture until the pH was 4, then the mixture was diluted with water (5 mL) and extracted with ethyl acetate (3×15 mL). The combined organic layers were dried over anhydrous sodium sulfate, filtered and the filtrate was concentrated in vacuo to give the title compound (1.7 g) as a yellow solid. LC-MS (ESI+) m/z 561.2 (M+1)⁺.

### Step 6—Tert-butyldimethylsilyl 2-(2-(((tert-butyldimethylsilyl)oxy)methyl)phenoxy)-4-(4-((4′-chloro-5,5-dimethyl-3,4,5,6-tetrahydro-[1,1′-biphenyl]-2-yl)methyl)piperazin-1-yl)benzoate

To a solution of 4-(4-((4′-chloro-5,5-dimethyl-3,4,5,6-tetrahydro-[1,1′-biphenyl]-2-yl)methyl)piperazin-1-yl)-2-(2-(hydroxymethyl)phenoxy)benzoic acid (1.7 g, 3.0 mmol) in anhydrous DCM (20 mL) was added TEA (1.23 g, 12.1 mmol) at 25° C. Then TBSOTf (3.20 g, 12.1 mmol) was added at 0° C., and the reaction was stirred at 25° C. for 0.5 hr. On completion, the reaction mixture was quenched with water (8 mL) and extracted with dichloromethane (3×15 mL). The combined organic layers were dried over anhydrous sodium sulfate, filtered and the filtrate was concentrated in vacuo to give the title compound (1.8 g) as a green oil. LC-MS (ESI+) m/z 789.6 (M+1)⁺.

### Synthesis of 4-((3-(Piperazin-1-yl)propyl)amino)-3-((trifluoromethyl)sulfonyl)benzenesulfonamide (Intermediate UJ)

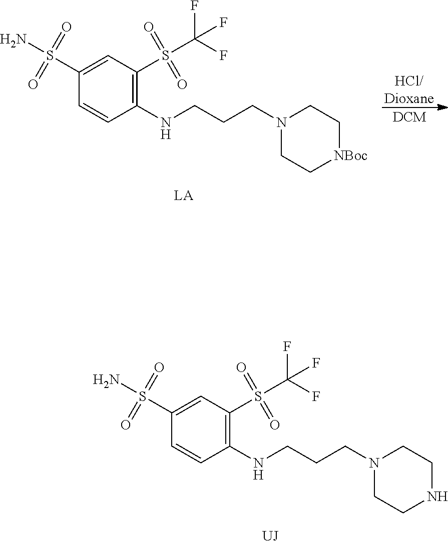

To a solution of tert-butyl 4-(3-((4-sulfamoyl-2-((trifluoromethyl)sulfonyl)phenyl)amino)propyl)piperazine-1-carboxylate (500 mg, 942μ mol, Intermediate LA) in anhydrous DCM (1 mL) was added HCl/dioxane (4 M, 3.2 mL) at 25° C., then the reaction was stirred at for 2 hrs. On completion, the reaction mixture was concentrated in vacuo to give the title compound (500 mg) as a white solid. LC-MS (ESI+) m/z 431.1 (M+1)⁺.

### Synthesis of Ethyl 7-oxo-7-(4-(3-((4-sulfamoyl-2-((trifluoromethyl)sulfonyl)phenyl)amino)propyl)piperazin-1-yl)heptanoate (Intermediate UK)

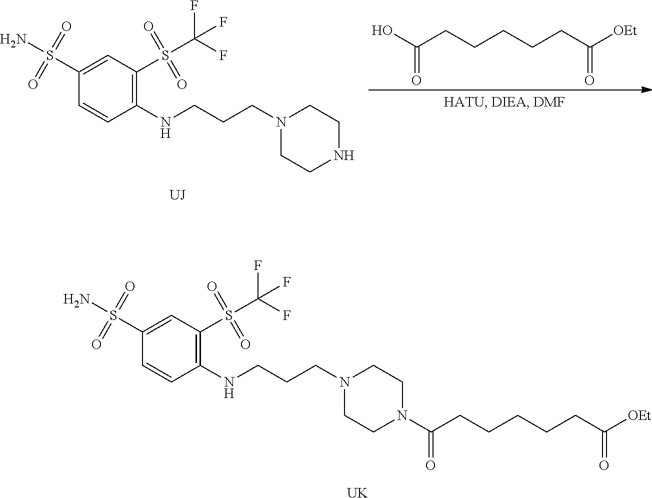 [SMILES: NS(=O)(=O)c1ccc(NCCCN2CCNCC2)c(S(=O)(=O)C(F)(F)F)c1 ; CCOC(=O)CCCCCC(=O)O ; CCOC(=O)CCCCCC(=O)N1CCN(CCCNc2ccc(S(N)(=O)=O)cc2S(=O)(=O)C(F)(F)F)CC1 ; "UK"]

To a solution of 4-((3-(piperazin-1-yl)propyl)amino)-3-((trifluoromethyl)sulfonyl)benzenesulfonamide (440 mg, 942 μmol, Intermediate UJ) and 7-ethoxy-7-oxoheptanoic acid (212 mg, 1.13 mmol, CAS #33018-91-6) in DMF (2 mL) was added HATU (537 mg, 1.41 mmol) and DIEA (365 mg, 2.83 mmol) at 25° C., then the mixture was stirred at 25° C. for 10 mins. On completion, the reaction mixture was quenched with water (5 mL) and extracted with ethyl acetate (3×8 mL). The combined organic layers were washed with brine (10 mL) and dried over anhydrous sodium sulfate, filtered and the filtrate was concentrated in vacuo to give the crude residue. The residue was purified by column chromatography (SiO₂, DCM:MeOH=20:1) to give the title compound (500 mg, 83% yield) as a green oil. LC-MS (ESI+) m/z 601.1 (M+1)⁺.

### Synthesis of 7-(4-(3-((4-(N-(2-(2-(((tert-butyldimethylsilyl)oxy)methyl)phenoxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-(trifluoromethyl)sulfonyl)phenyl)amino)propyl)piperazin-1-yl)-7-oxoheptanoic Acid (Intermediate UL)

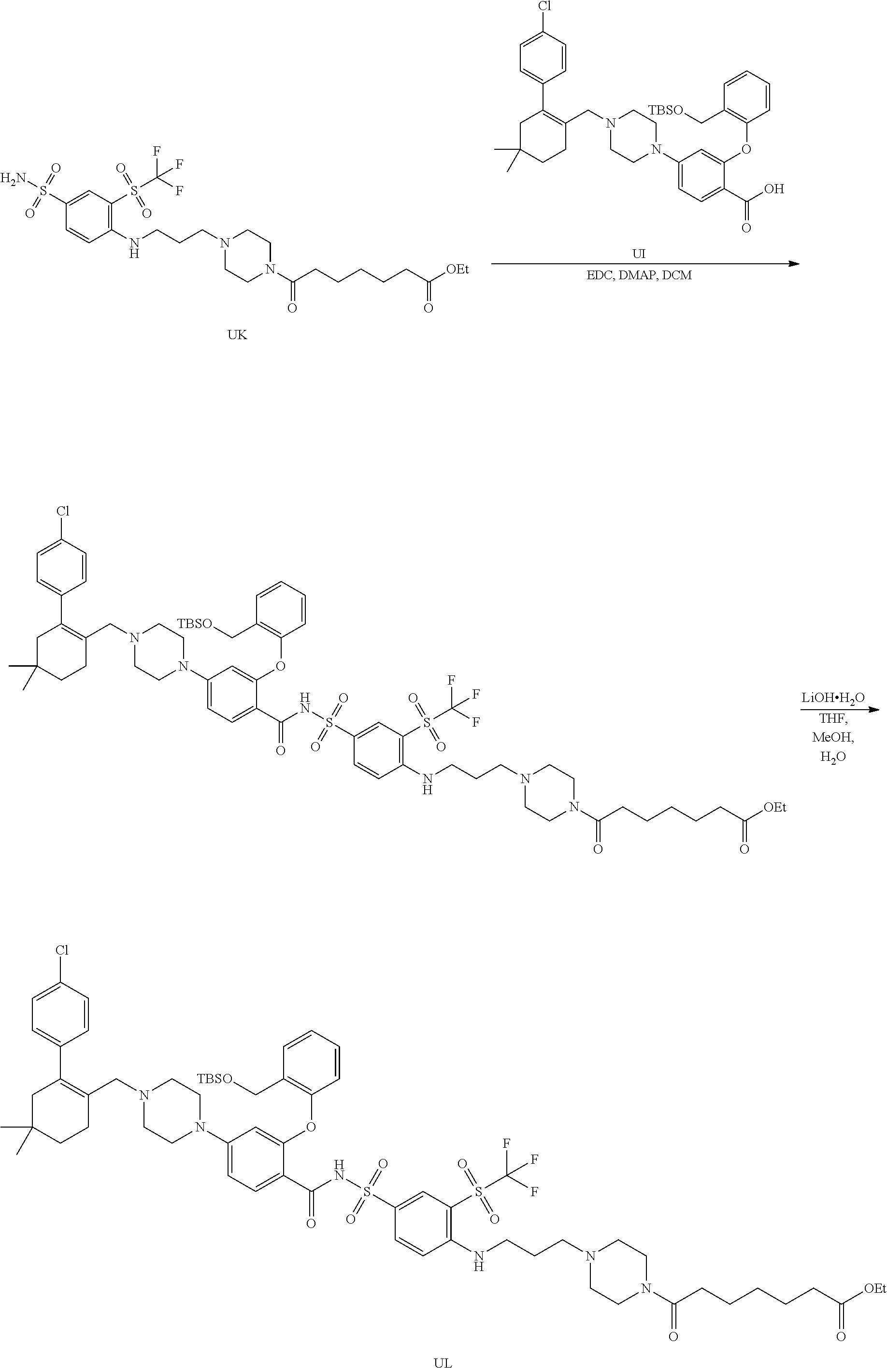

### Step 1—Ethyl 7-(4-(3-((4-(N-(2-(2-(((tert-butyldimethylsilyl)oxy)methyl)phenoxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)-7-oxoheptanoate

To a solution of 2-(2-(((tert-butyldimethylsilyl)oxy)methyl)phenoxy)-4-(4-((4′-chloro-5,5-dimethyl-3,4,5,6-tetrahydro-[1,1′-biphenyl]-2-yl)methyl)piperazin-1-yl)benzoic acid (500 mg, 740 μmol, Intermediate UI) in anhydrous DCM (5 mL) was added EDC (287 mg, 1.85 mmol), DMAP (271 mg, 2.22 mmol) and ethyl 7-oxo-7-(4-(3-((4-sulfamoyl-2-((trifluoromethyl)sulfonyl)phenyl)amino)propyl)piperazin-1-yl)heptanoate (444 mg, 740 μmol, Intermediate UK) at 25° C., then the reaction was stirred at 25° C. for 2 hrs. On completion, the reaction mixture was quenched with water (5 mL) and extracted with ethyl acetate (3×8 mL). The combined organic layers were washed with brine (8 mL) and dried over anhydrous sodium sulfate, filtered and the filtrate was concentrated in vacuo to give the crude residue. The residue was purified by column chromatography (SiO₂, DCM:MeOH=30:1) to give the title compound (220 mg) as a yellow solid. LC-MS (ESI+) m/z 629.7 (M+1)⁺.

### Step 2—7-(4-(3-((4-(N-(2-(2-(((tert-butyldimethylsilyl)oxy)methyl)phenoxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)-7-oxoheptanoic Acid

A solution of ethyl 7-(4-(3-((4-(N-(2-(2-(((tert-butyldimethylsilyl)oxy)methyl)phenoxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)-7-oxoheptanoate (160 mg, 127 μmol) and LiOH·H₂O (26.6 mg, 635 μmol) in THF (0.1 mL), MeOH (0.1 mL) and H₂O (0.1 mL) was stirred at 0° C. for 2 hrs. On completion, HCl (1N) was added to the reaction mixture until the pH was 7, then diluted with water (10 mL) and extracted with ethyl acetate (3×10 mL). The combined organic layers were dried over anhydrous sodium sulfate, filtered and the filtrate was concentrated in vacuo to give the crude residue. The crude residue was purified by reversed-phase HPLC (0.8 g/L ammonium bicarbonate) to give the title compound (120 mg, 75% yield) as a white solid. LC-MS (ESI+) m/z 1229.1 (M+1)⁻.

### Synthesis of 2-((1H-pyrrolo[2,3-b]pyridin-5-yl)oxy)-4-(4-((1-(tert-butoxycarbonyl)-5-(4-chlorophenyl)-1,2,3,6-tetrahydropyridin-4-yl)methyl)piperazin-1-yl)benzoic Acid (Intermediate UM)

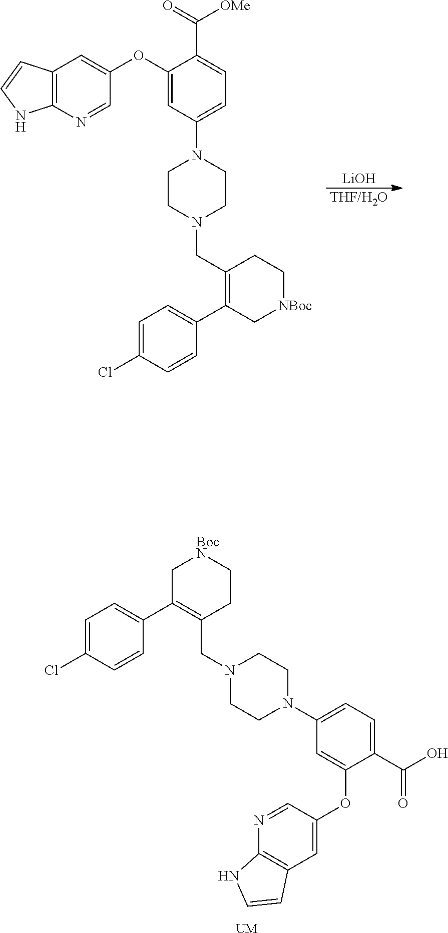

To a solution of tert-butyl 5-(4-chlorophenyl)-4-[[4-[4-methoxycarbonyl-3-(1H-pyrrolo[2,3-b]pyridin-5-yloxy)phenyl]piperazin-1-yl]methyl]-3,6-dihydro-2H-pyridine-1-carboxylate (500 mg, 760 μmol, synthesized via Step 1 of Intermediate QT) in THF (4 mL), MeOH (2 mL) and H₂O (2 mL) was added LiOH H₂O (159 mg, 3.80 mmol). The mixture was then stirred at 25° C. for 2 hrs. On completion, the reaction mixture was diluted with H₂O (10 mL) and extracted with EA (10 mL×3). The combined organic layers were dried over Na₂SO₄, filtered and concentrated under reduced pressure to give the title compound (490 mg) as a yellow solid. LC-MS (ESI⁺) m/z 644.3 (M+H)⁺.

### Synthesis of 7-(4-(3-((4-(N-(2-((1H-pyrrolo[2,3-b]pyridin-5-yl)oxy)-4-(4-((1-(tert-butoxycarbonyl)-5-(4-chlorophenyl)-1,2,3,6-tetrahydropyridin-4-yl)methyl)piperazin-1-yl)benzoyl)sulfamoyl)-2-((trifluoromethyl)sulfonyl)phenyl)amino)propyl)piperazin-1-yl)-7-oxoheptanoic Acid (Intermediate UN)

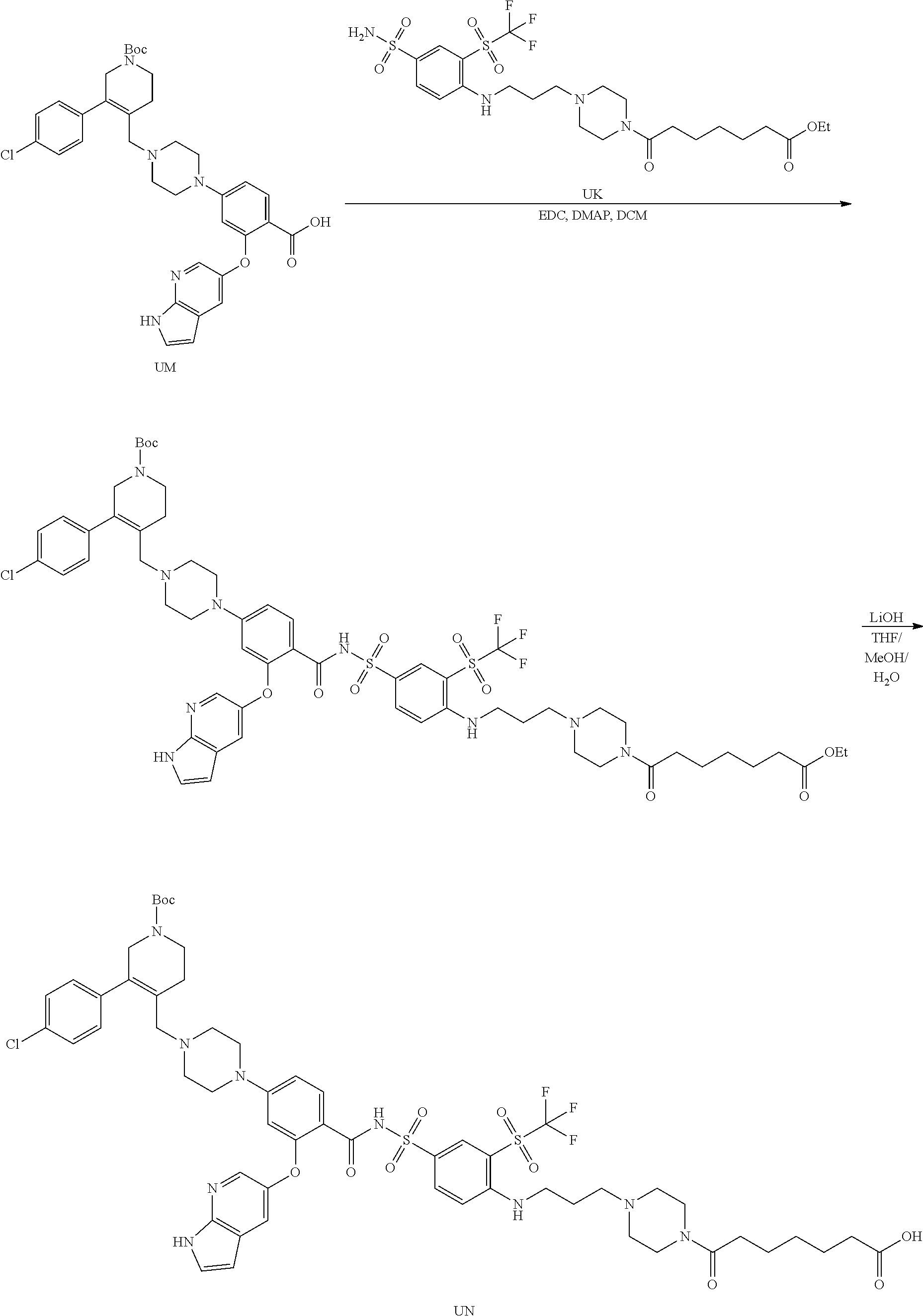

### Step 1—Tert-butyl 4-((4-(3-((1H-pyrrolo[2,3-b]pyridin-5-yl)oxy)-4-(((4-((3-(4-(7-ethoxy-7-oxoheptanoyl)piperazin-1-yl)propyl)amino)-3-((trifluoromethyl)sulfonyl)phenyl)sulfonyl)carbamoyl)phenyl)piperazin-1-yl)methyl)-5-(4-chlorophenyl)-3,6-dihydropyridine-1(2H)-carboxylate

To a solution of 4-[4-[[1-tert-butoxycarbonyl-5-(4-chlorophenyl)-3,6-dihydro-2H-pyridin-4-yl]methyl]piperazin-1-yl]-2-(1H-pyrrolo[2,3-b]pyridin-5-yloxy)benzoic acid (260 mg, 404 μmol, Intermediate UM) in DCM (4 mL) was added EDC (62.7 mg, 404 μmol) and DMAP (49.3 mg, 404 μmol) at 0° C. Then ethyl 7-oxo-7-[4-[3-[4-sulfamoyl-2-(trifluoromethylsulfonyl)anilino]propyl]piperazin-1-yl]heptanoate (242 mg, 404 μmol, Intermediate UK) was added and the mixture was stirred at 25° C. for 12 hrs. On completion, the reaction mixture was washed with (4 mL×2) H2O, and then the organic layer was concentrated under reduced pressure to give a residue. The residue was purified by prep-HPLC (0.1% FA condition) to give the title compound (220 mg, 44% yield) as a white solid. LC-MS (ESI⁺) m/z 614.3 (M+2H)⁺/2. ¹H NMR (400 MHz, DMSO-d₆) δ=11.58 (s, 1H), 8.15 (s, 1H), 8.01 (s, 1H), 7.95 (d, J=2.4 Hz, 1H), 7.76 (d, J=8.4 Hz, 1H), 7.54 (d, J=8.4 Hz, 1H), 7.44 (t, J=2.8 Hz, 1H), 7.41 (d, J=8.4 Hz, 2H), 7.34 (s, 1H), 7.17 (d, J=8.4 Hz, 2H), 7.07 (d, J=1.6 Hz, 1H), 6.80 (d, J=8.8 Hz, 1H), 6.63 (dd, J=1.6, 8.8 Hz, 1H), 6.33 (dd, J=1.6, 3.2 Hz, 1H), 6.22 (d, J=1.6 Hz, 1H), 4.03 (dd, J=2.4, 7.2 Hz, 2H), 3.89 (s, 2H), 3.44 (d, J=11.6 Hz, 7H), 3.32 (s, 1H), 3.00 (s, 4H), 2.75 (s, 2H), 2.37-2.31 (m, 4H), 2.30-2.23 (m, 7H), 2.20 (s, 5H), 1.75-1.65 (m, 2H), 1.54-1.43 (m, 4H), 1.39 (s, 9H), 1.29-1.23 (m, 2H), 1.18-1.15 (m, 3H).

### Step 2—7-(4-(3-((4-(N-(2-((1H-pyrrolo[2,3-b]pyridin-5-yl)oxy)-4-(4-((1-(tert-butoxycarbonyl)-5-(4-chlorophenyl)-1,2,3,6-tetrahydropyridin-4-yl)methyl)piperazin-1-yl)benzoyl)sulfamoyl)-2-((trifluoromethyl)sulfonyl)phenyl)amino)propyl)piperazin-1-yl)-7-oxoheptanoic Acid

To a solution of tert-butyl 5-(4-chlorophenyl)-4-[[4-[4-[[4-[3-[4-(7-ethoxy-7-oxo-heptanoyl)piperazin-1-yl]propylamino]-3-(trifluoromethylsulfonyl)phenyl]sulfonylcarbamoyl]-3-(1H-pyrrolo[2,3-b]pyridin-5-yloxy)phenyl]piperazin-1-yl]methyl]-3,6-dihydro-2H-pyridine-1-carboxylate (220 mg, 179 μmol) in THF (4 mL), MeOH (2 mL), and H₂O (2 mL) was added LiOH·H₂O (37.6 mg, 897 μmol) The mixture was then stirred at 25° C. for 2 hrs. On completion, HCl (1N) was added to the the reaction mixture until the pH was 7, then diluted by water (8 mL) and extracted by ethyl acetate (3×10 mL), The combined organic layers were dried over anhydrous sodium sulfate, filtered and the filtrate was concentrated in vacuo to give the title compound (197 mg) as a yellow solid. LC-MS (ESI⁺) m/z 1198.5 (M+H)⁺.

### Synthesis of (R)-2-((1H-pyrrolo[2,3-b]pyridin-5-yl)oxy)-4-(4-((4-(((tert-butoxycarbonyl)amino)methyl)-4′-chloro-4-methyl-3,4,5,6-tetrahydro-[1,1′-biphenyl]-2-yl)methyl)piperazin-1-yl)benzoic Acid (Intermediate UO)

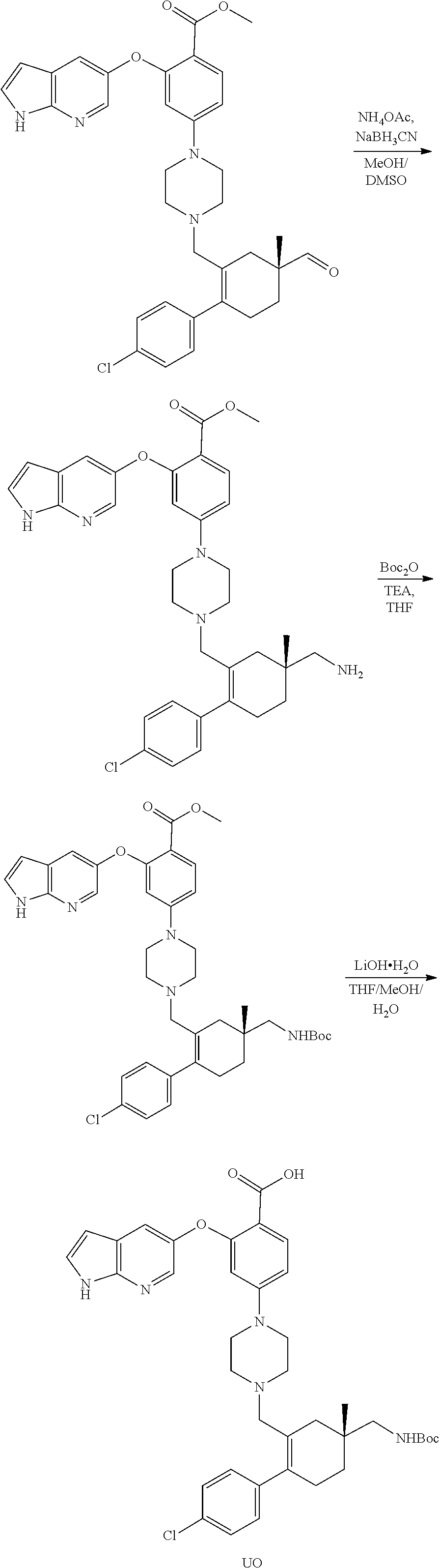

### Step 1—Methyl (R)-2-((1H-pyrrolo[2,3-b]pyridin-5-yl)oxy)-4-(4-((4-(aminomethyl)-4′-chloro-4-methyl-3,4,5,6-tetrahydro-[1,1′-biphenyl]-2-yl)methyl)piperazin-1-yl)benzoate

To a solution of methyl (R)-2-((1H-pyrrolo[2,3-b]pyridin-5-yl)oxy)-4-(4-((4′-chloro-4-formyl-4-methyl-3,4,5,6-tetrahydro-[1,1′-biphenyl]-2-yl)methyl)piperazin-1-yl)benzoate (500 mg, 835 μmol, synthesized via Steps 1-2 of Intermediate NU) in MeOH (8 mL) and DMSO (2 mL) was added NH₄OAc (643 mg, 8.35 mmol) and NaBH₃CN (210 mg, 3.34 mmol) at 0° C. The mixture was then stirred at 25° C. for 4 hrs. On completion, the reaction mixture was filtered to give the compound (40 mg) as a brown solid. LC-MS (ESI+) m/z 600.3 (M+H).

### Step 2—Methyl (R)-2-((1H-pyrrolo[2,3-b]pyridin-5-yl)oxy)-4-(4-((4-(((tert-butoxycarbonyl)amino)methyl)-4′-chloro-4-methyl-3,4,5,6-tetrahydro-[1,1′-biphenyl]-2-yl)methyl)piperazin-1-yl)benzoate

To a solution of methyl (R)-2-((1H-pyrrolo[2,3-b]pyridin-5-yl)oxy)-4-(4-((4-(aminomethyl)-4′-chloro-4-methyl-3,4,5,6-tetrahydro-[1,1′-biphenyl]-2-yl)methyl)piperazin-1-yl)benzoate (40 mg, 66.7 μmol) in THF (6 mL) was added Boc₂O (21.8 mg, 99.9 μmol, 22.9 uL) and TEA (20.2 mg, 199 μmol, 27.8 uL). The mixture was then stirred at 25° C. for 12 hrs. On completion, the reaction mixture was concentrated under reduced pressure to give a residue. The residue was purified by reversed-phase HPLC (0.8 g/L ammonium bicarbonate) to give the title compound (55 mg, 15% yield) as a white solid. LC-MS (ESI⁺) m/z 700.3 (M+H)⁻.

### Step 3—(R)-2-((1H-pyrrolo[2,3-b]pyridin-5-yl)oxy)-4-(4-((4-(((tert-butoxycarbonyl)amino)methyl)-4′-chloro-4-methyl-3,4,5,6-tetrahydro-[1,1′-biphenyl]-2-yl)methyl)piperazin-1-yl)benzoic Acid

To a solution of methyl (R)-2-((1H-pyrrolo[2,3-b]pyridin-5-yl)oxy)-4-(4-((4-(((tert-butoxycarbonyl)amino)methyl)-4′-chloro-4-methyl-3,4,5,6-tetrahydro-[1,1′-biphenyl]-2-yl)methyl)piperazin-1-yl)benzoate (55 mg, 78.5 μmol) in THF (4 mL), MeOH (1 mL) and H₂O (1 mL) was added LiOH·H₂O (16.5 mg, 393 μmol). The mixture was then stirred at 50° C. for 12 hrs. On completion, HCl (1N) was added to the reaction mixture until the pH=5, then the mixture was diluted with water (1 mL) and extracted with dichloromethane (3×3 mL). The combined organic layers were dried over anhydrous sodium sulfate, filtered and the filtrate was concentrated in vacuo to give the crude residue to give the title compound (70 mg) as a gray solid. LC-MS (ESI⁺) m/z 686.3 (M+H)⁺.

### Synthesis of (R)-2-((1H-pyrrolo[2,3-b]pyridin-5-yl)oxy)-4-(4-((4-(aminomethyl)-4′-chloro-4-methyl-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide (Intermediate UP)

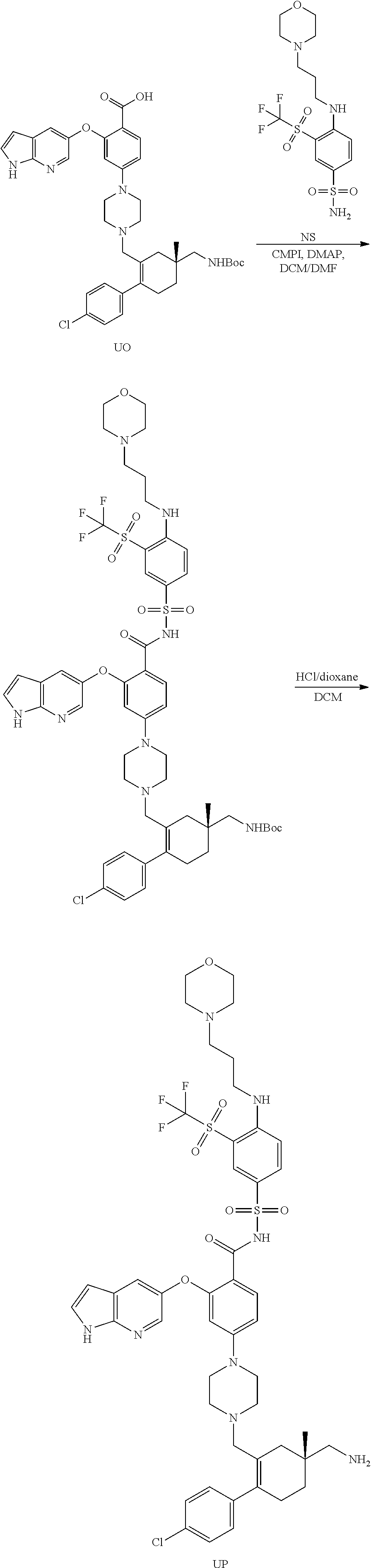

### Step 1—Tert-butyl (R)-((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)carbamate

To a solution of (R)-2-((1H-pyrrolo[2,3-b]pyridin-5-yl)oxy)-4-(4-((4-(((tert-butoxycarbonyl)amino)methyl)-4′-chloro-4-methyl-3,4,5,6-tetrahydro-[1,1′-biphenyl]-2-yl)methyl)piperazin-1-yl)benzoic acid (50 mg, 70 μmol, Intermediate UO) in DCM (2.5 mL) was added DMAP (8.90 mg, 72.9 μmol), TEA (22.1 mg, 219 μmol, 30.4 uL) and CMPI (27.9 mg, 109 μmol). Then 4-((3-morpholinopropyl)amino)-3-((trifluoromethyl)sulfonyl)benzenesulfonamide (40.9 mg, 94.7 μmol, Intermediate NS) was added at 0° C. and the mixture was stirred at 25° C. for 12 hrs. On completion, the reaction mixture was filtered to give a filtrate. The filtrate was purified by reversed-phase HPLC (0.8 g/L ammonium bicarbonate) to give the title compound (40 mg, 48% yield) as a white solid. LC-MS (ESI⁺) m/z 1100.8 (M+H)⁺.

### Step 2—(R)-2-((1H-pyrrolo[2,3-b]pyridin-5-yl)oxy)-4-(4-((4-(aminomethyl)-4′-chloro-4-methyl-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide

To a solution of tert-butyl (R)-((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)carbamate (40 mg, 40 μmol) in DCM (4 mL) was added HCl/dioxane (4 M, 0.5 mL). The mixture was then stirred at 25° C. for 1 hr. On completion, the reaction mixture was concentrated under reduced pressure to give the title compound (40 mg, HCl) as a gray solid. LC-MS (ESI⁺) m/z 999.2 (M+H)⁺.

### Synthesis of 2-((1H-pyrrolo[2,3-b]pyridin-5-yl)oxy)-6-(4-((4′-chloro-5,5-dimethyl-3,4,5,6-tetrahydro-[1,1′-biphenyl]-2-yl)methyl)piperazin-1-yl)nicotinic Acid (Intermediate UQ)

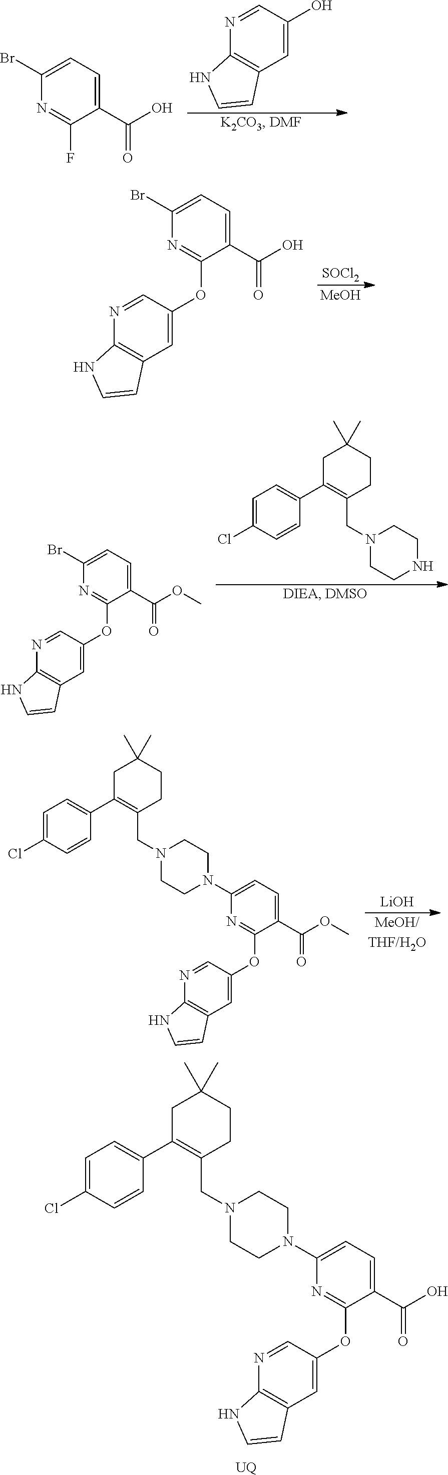

### Step 1—2-((1H-pyrrolo[2,3-b]pyridin-5-yl)oxy)-6-bromonicotinic Acid

To a solution of 6-bromo-2-fluoro-pyridine-3-carboxylic acid (3 g, 13.6 mmol, CAS #1214345-17-1) in DMF (20 mL) was added K₂CO₃ (5.65 g, 40.9 mmol) and 1H-pyrrolo[2,3-b]pyridin-5-ol (1.83 g, 13.6 mmol, CAS #90929-73-0). The mixture was then stirred at 70° C. for 12 hrs. On completion, HCl (1N) was added to the reaction mixture until the pH 3-5, then the mixture was diluted with water (20 mL) and extracted with ethyl acetate (3×50 mL). The combined organic layers were dried over anhydrous sodium sulfate, filtered and the filtrate was concentrated in vacuo to give the crude residue. The residue was purified by column chromatography (SiO₂, Petroleum ether:Ethyl acetate=10/0 to 5/1) to give the title compound (4 g, 85% yield) as a yellow solid. LC-MS (ESI⁺) m/z 334.1 (M)⁺.

### Step 2—Methyl 2-((1H-pyrrolo[2,3-b]pyridin-5-yl)oxy)-6-bromonicotinate

A solution of 6-bromo-2-(1H-pyrrolo[2,3-b]pyridin-5-yloxy)pyridine-3-carboxylic acid (500 mg, 1.50 mmol) in MeOH (6 mL) was cooled to 0° C. then SOCl₂ (534 mg, 4.49 mmol, 325 uL) was added dropwise. The mixture was degassed and purged with N₂ three times and then the mixture was stirred at 80° C. for 4 hrs under N₂ atmosphere. On completion, the reaction mixture was concentrated under reduced pressure to give the title compound (400 mg) as a gray solid. LC-MS (ESI⁺) m/z 349.8 (M+H)⁺.

### Step 3—Methyl 2-((1H-pyrrolo[2,3-b]pyridin-5-yl)oxy)-6-(4-((4′-chloro-5,5-dimethyl-3,4,5,6-tetrahydro-[1,1′-biphenyl]-2-yl)methyl)piperazin-1-yl)nicotinate

A mixture of methyl 6-bromo-2-(1H-pyrrolo[2,3-b]pyridin-5-yloxy)pyridine-3-carboxylate (300 mg, 861 μmol), 1-[[2-(4-chlorophenyl)-4,4-dimethyl-cyclohexen-1-yl]methyl]piperazine (274 mg, 861 μmol, CAS #1214345-17-1), and DIEA (334 mg, 2.59 mmol, 450 uL) in DMSO (5 mL) was degassed and purged with N₂ three times. Then the mixture was stirred at 70° C. for 3 hrs under N₂ atmosphere. On completion, the reaction mixture was quenched by addition of H₂O (10 mL) and extracted with EA (10 mL×3). The combined organic layers were washed with brine (10 mL) and dried over anhydrous sodium sulfate, filtered and the filtrate was concentrated in vacuo to give the title compound (400 mg) as yellow solid. LC-MS (ESI⁺) m/z 586.3 (M+H)⁺.

### Step 4—2-((1H-pyrrolo[2,3-b]pyridin-5-yl)oxy)-6-(4-((4′-chloro-5,5-dimethyl-3,4,5,6-tetrahydro-[1,1′-biphenyl]-2-yl)methyl)piperazin-1-yl)nicotinic Acid

To a solution of methyl 6-[4-[[2-(4-chlorophenyl)-4,4-dimethyl-cyclohexen-1-yl]methyl]piperazin-1-yl]-2-(1Hpyrrolo[2,3-b]pyridin-5-yloxy)pyridine-3-carboxylate (400 mg, 682 μmol) in MeOH (1 mL), H₂O (1 mL) and THF (1 mL) was added LiOH·H₂O (286 mg, 6.82 mmol). The mixture was then stirred at 25° C. for 2 hrs. On completion, the reaction mixture was filtered and the filtrate was concentrated in vacuo to give the crude residue. On completion, the crude product was purified by reversed-phase HPLC (0.1% FA condition) to give the title compound (340 mg, 83% yield, FA) as a white solid. LC-MS (ESI⁺) m/z 572.2 (M+H)⁺.

### Synthesis of 2-((1H-pyrrolo[2,3-b]pyridin-5-yl)oxy)-6-(4-((4′-chloro-5,5-dimethyl-3,4,5,6-tetrahydro-[1,1′-biphenyl]-2-yl)methyl)piperazin-1-yl)-N-((4-((3-(piperazin-1-yl)propyl)amino)-3-((trifluoromethyl)sulfonyl)phenyl)sulfonyl)nicotinamide (Intermediate UR)

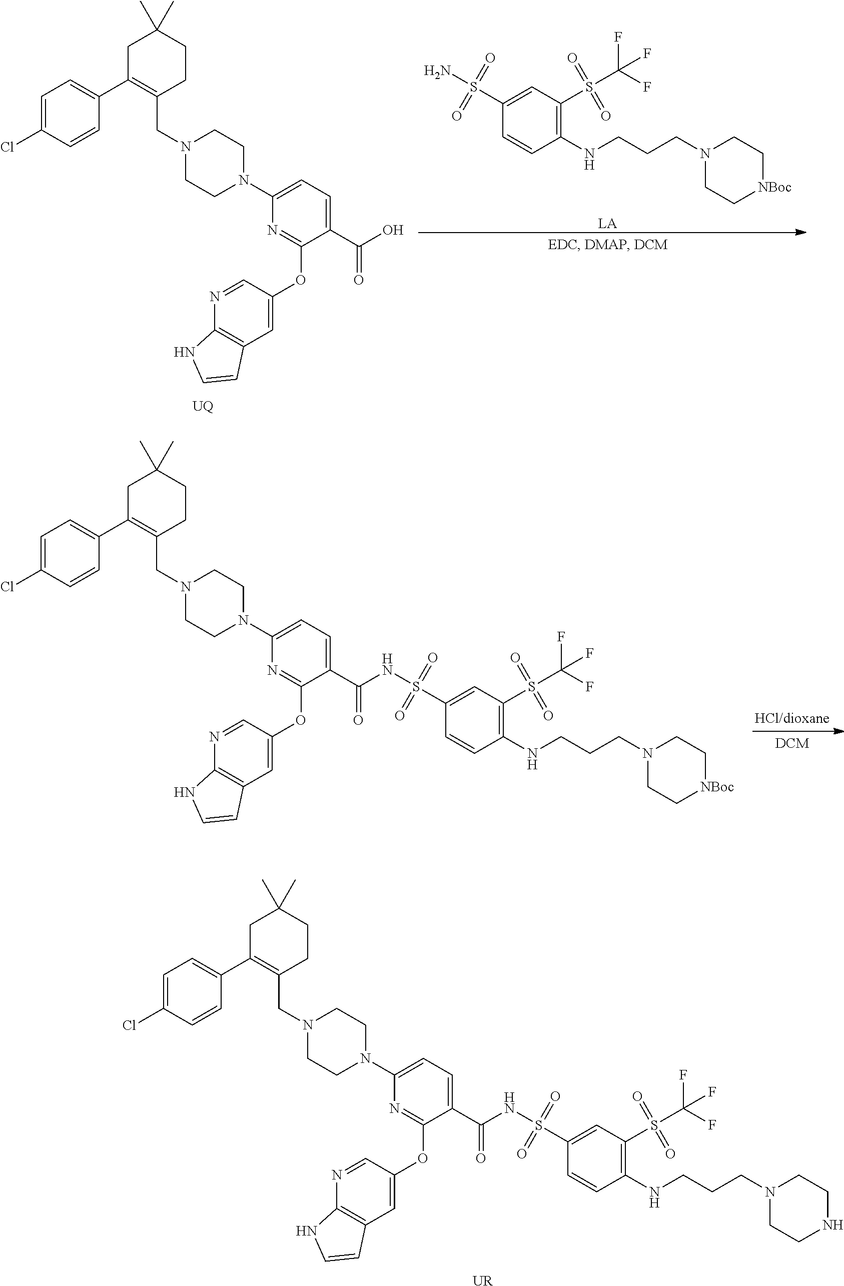

### Step 1—Tert-butyl 4-(3-((4-(N-(2-((1H-pyrrolo[2,3-b]pyridin-5-yl)oxy)-6-(4-((4′-chloro-5,5-dimethyl-3,4,5,6-tetrahydro-[1,1′-biphenyl]-2-yl)methyl)piperazin-1-yl) nicotinoyl)sulfamoyl)-2-((trifluoromethyl)sulfonyl)phenyl)amino)propyl)piperazine-1-carboxylate

To a solution of 6-[4-[[2-(4-chlorophenyl)-4,4-dimethyl-cyclohexen-1-yl]methyl]piperazin-1-yl]-2-(1H-pyrrolo[2,3-b]pyridin-5-yloxy)pyridine-3-carboxylic acid (170 mg, 297 μmol, Intermediate UQ) in DCM (2 mL) was added DMAP (90.7 mg, 742 μmol), EDC (92.2 mg, 594 μmol, 105 uL), and tert-butyl 4-[3-[4-sulfamoyl-2-(trifluoromethylsulfonyl)anilino]propyl]piperazine-1-carboxylate (157 mg, 297 μmol, Intermediate LA). The mixture was then stirred at 30° C. for 3 hrs. On completion, the reaction mixture was filtered and the filtrate was concentrated in vacuo to give the crude residue. On completion, the crude product was purified by reversed-phase HPLC (0.1% FA condition) to give the title compound (240 mg, 64% yield, FA) as a white solid. HR-MS (ESI⁺) m/z 543.2 (M/2+H)⁺.

### Step 2—2-((1H-pyrrolo[2,3-b]pyridin-5-yl)oxy)-6-(4-((4′-chloro-5,5-dimethyl-3,4,5,6-tetrahydro-[1,1′-biphenyl]-2-yl)methyl)piperazin-1-yl)-N-((4-((3-(piperazin-1-yl)propyl)amino)-3-((trifluoromethyl)sulfonyl)phenyl)sulfonyl)nicotinamide

To a solution of tert-butyl 4-[3-[4-[[6-[4-[[2-(4-chlorophenyl)-4,4-dimethyl-cyclohexen-1-yl]methyl]piperazin-1-yl]-2-(1H-pyrrolo[2,3-b]pyridin-5-yloxy)pyridine-3-carbonyl]sulfamoyl]-2-(trifluoromethylsulfonyl)anilino]propyl]piperazine-1-carboxylate (200 mg, 184 μmol) in DCM (10 mL) was added HCl/dioxane (4 M, 2.00 mL). The mixture was then stirred at 25° C. for 2 hrs. On completion, the reaction mixture was concentrated under reduced pressure to give the title compound (200 mg, HCl) as a gray solid. LC-MS (ESI⁺) m/z 984.4 (M+H)⁺.

### Synthesis of 6-(9-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)-3,9-diazaspiro[5,5]undecan-3-yl)hexanoic Acid (Intermediate US)

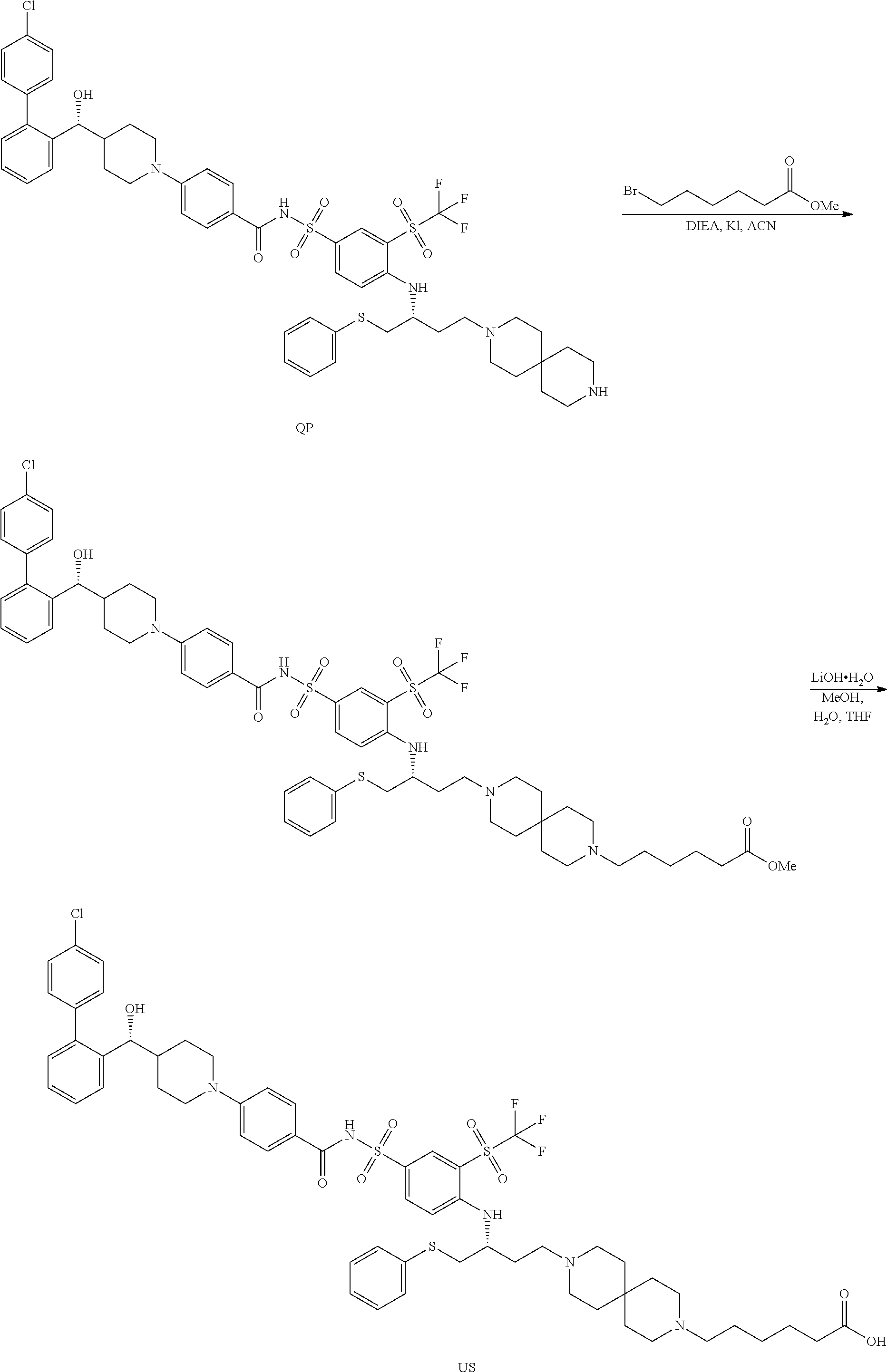

### Step 1—Methyl 6-(9-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)-3,9-diazaspiro[5.5]undecan-3-yl)hexanoate

To a solution of 4-(4-((R)-(4′-chloro-[1,1′-biphenyl]-2-yl)(hydroxy)methyl) piperidin-1-yl)-N-((4-(((R)-1-(phenylthio)-4-(3,9-diazaspiro[5.5]undecan-3-yl)butan-2-yl)amino)-3-((trifluoromethyl)sulfonyl)phenyl)sulfonyl)benzamide (200 mg, 195 μmol, Intermediate QP) in ACN (4 mL) was added methyl 6-bromohexanoate (81.6 mg, 390 μmol, CAS #14273-90-6), DIEA (252 mg, 1.95 mmol, 340 uL) and KI (4.86 mg, 29.3 μmol). The reaction was then stirred at 50° C. for 12 hrs. On completion, the reaction mixture was concentrated in vacuo to give the crude residue. The crude residue was purified by reversed-phase HPLC (0.1% FA condition) to give the title compound (150 mg, 66% yield) as a white solid. LC-MS (ESI+) m/z 1152.4. (M+H)⁺.

### Step 2—6-(9-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)-3,9-diazaspiro[5.5]undecan-3-yl)hexanoic Acid

To a solution of methyl 6-(9-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)-3,9-diazaspiro[5.5]undecan-3-yl)hexanoate (150 mg, 130 μmol) in THF (2 mL), H₂O (0.5 mL) and MeOH (0.5 mL) was added LiOH·H₂O (27.3 mg, 651 μmol). The reaction was then stirred at 25° C. for 1 hr. On completion, HCl (1N) was added to the reaction mixture until the pH 7, then concentrated in vacuo and lyophilized to give the title compound (120 mg) as a white solid. LC-MS (ESI+) m/z 569.9. (M+H)⁺.

### Synthesis of 4-((3-((2-((Tert-butyldimethylsilyl)oxy)ethyl)(methyl)amino)propyl)amino)-3-((trifluoromethyl)sulfonyl)benzenesulfonamide (Intermediate UT)

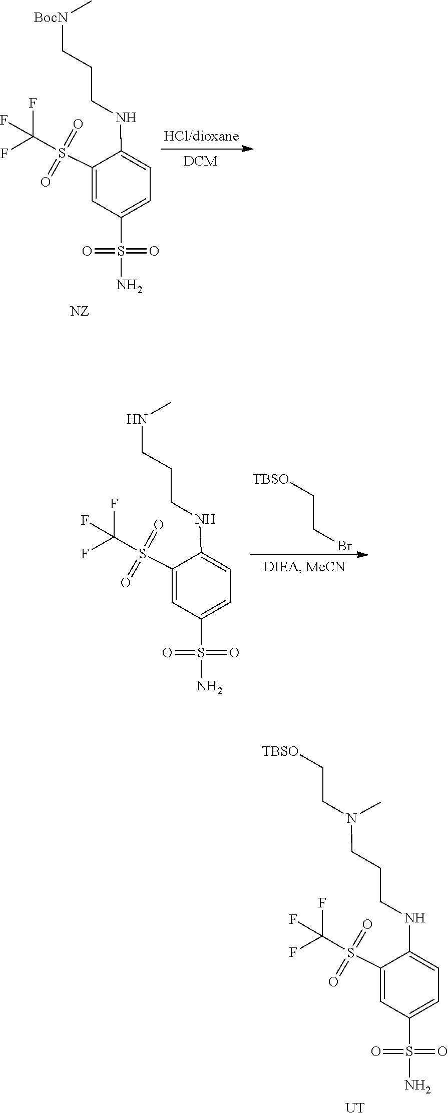 [SMILES: CN(CCCNc1ccc(S(N)(=O)=O)cc1S(=O)(=O)C(F)(F)F)C(=O)OC(C)(C)C ; CNCCCNc1ccc(S(N)(=O)=O)cc1S(=O)(=O)C(F)(F)F ; CC(C)(C)[Si](C)(C)OCCBr ; CN(CCCNc1ccc(S(N)(=O)=O)cc1S(=O)(=O)C(F)(F)F)CCO[Si](C)(C)C(C)(C)C ; "UT"]

### Step 1—4-((3-(Methylamino)propyl)amino)-3-((trifluoromethyl)sulfonyl)benzenesulfonamide

To a solution of tert-butyl methyl (3-((4-sulfamoyl-2-((trifluoromethyl)sulfonyl)phenyl)amino)propyl)carbamate (1 g, 2.10 mmol, Intermediate NZ) in DCM (10 mL) was added HCl/dioxane (4 M, 525 uL). The reaction was then stirred at 25° C. for 30 mins. On completion, the reaction mixture was concentrated under reduced pressure to give the title compound (800 mg) as a white solid. LC-MS (ESI⁺) m/z 376.1. (M+H)⁺.

### Step 2—4-((3-((2-((Tert-butyldimethylsilyl)oxy)ethyl)(methyl)amino)propyl)amino)-3-((trifluoromethyl)sulfonyl)benzenesulfonamide

To a solution of 4-((3-((2-((tert-butyldimethylsilyl)oxy)ethyl)(methyl)amino)propyl)amino)-3-((trifluoromethyl)sulfonyl)benzenesulfonamide (600 mg, 1.60 mmol) and (2-bromoethoxy) (tert-butyl)dimethylsilane (764 mg, 3.20 mmol, CAS #86864-60-0) in ACN (10 mL) was added DIEA (2.07 g, 15.9 mmol, 2.78 mL). The reaction was then stirred at 70° C. for 12 hrs. On completion, the reaction mixture was concentrated under reduced pressure to give a residue. The residue was purified by column chromatography (SiO₂, Petroleum ether:Ethyl acetate=1/1 to Dichloromethan:Methanol=20/1) to give the title compound (680 mg, 72% yield) as a brown solid. LC-MS (ESI+) m/z 534.2. (M+H)⁺.

### Synthesis of (R)-2-((1H-pyrrolo[2,3-b]pyridin-5-yl)oxy)-4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-((3-((2-hydroxyethyl)(methyl)amino)propyl)amino)-3-((trifluoromethyl)sulfonyl)phenyl)sulfonyl)benzamide (Intermediate UU)

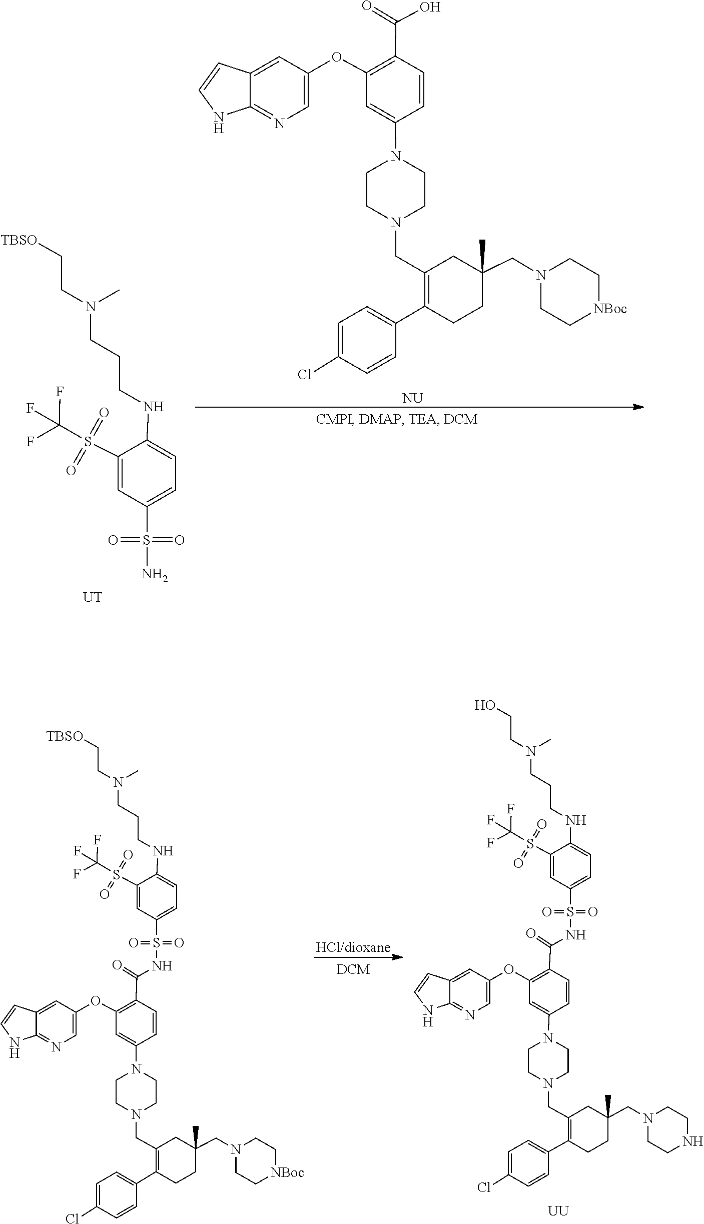

### Step 1—Tert-butyl (R)-4-((6-((4-(3-((1H-pyrrolo[2,3-b]pyridin-5-yl)oxy)-4-(((4-((3-((2-((tert- butyldimethylsilyl)oxy)ethyl)(methyl)amino)propyl)amino)-3-((trifluoromethyl) sulfonyl)phenyl) sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate

To a solution of (R)-2-((1H-pyrrolo[2,3-b]pyridin-5-yl)oxy)-4-(4-((4-((4-(tert-butoxycarbonyl)piperazin-1-yl)methyl)-4′-chloro-4-methyl-3,4,5,6-tetrahydro-[1,1′-biphenyl]-2-yl)methyl)piperazin-1-yl)benzoic acid (80 mg, 106 μmol, Intermediate NU) in DCM (1 mL) was added DMAP (12.9 mg, 106 μmol), CMPI (40.5 mg, 158 μmol), TEA (32.1 mg, 317 μmol, 44.2 uL) and 4-((3-((2-((tert-butyldimethylsilyl)oxy)ethyl)(methyl)amino)propyl)amino)-3-((trifluoromethyl)sulfonyl)benzenesulfonamide (62.1 mg, 116 μmol, Intermediate UT). The reaction was then stirred at 25° C. for 2 hrs. On completion, the reaction mixture was concentrated in vacuo to give the crude residue. The crude residue was purified by reversed-phase HPLC (0.1% FA condition) to give the title compound (100 mg, 60% yield) as a white solid. LC-MS (ESI⁺) m/z 1272.4. (M+H)⁺.

### Step 2—(R)-2-((1H-pyrrolo[2,3-b]pyridin-5-yl)oxy)-4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-((3-((2-hydroxyethyl)(methyl)amino)propyl)amino)-3-((trifluoromethyl)sulfonyl)phenyl)sulfonyl)benzamide

To a solution of tert-butyl (R)-4-((6-((4-(3-((1H-pyrrolo[2,3-b]pyridin-5-yl)oxy)-4-(((4-((3-((2-((tert-butyldimethylsilyl)oxy)ethyl)(methyl)amino)propyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazine-1-carboxylate (100 mg, 78.6 μmol) in DCM (1 mL) was added HCl/dioxane (4 M, 0.25 mL). The reaction was then stirred at 25° C. for 1 hr. On completion, the reaction mixture was concentrated in vacuo to give the title compound (80 mg) as a yellow solid. LC-MS (ESI+) m/z 1056.2. (M+H)⁺.

### Synthesis of 7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)-1,4-diazepan-1-yl)heptanoic Acid (Intermediate UV)

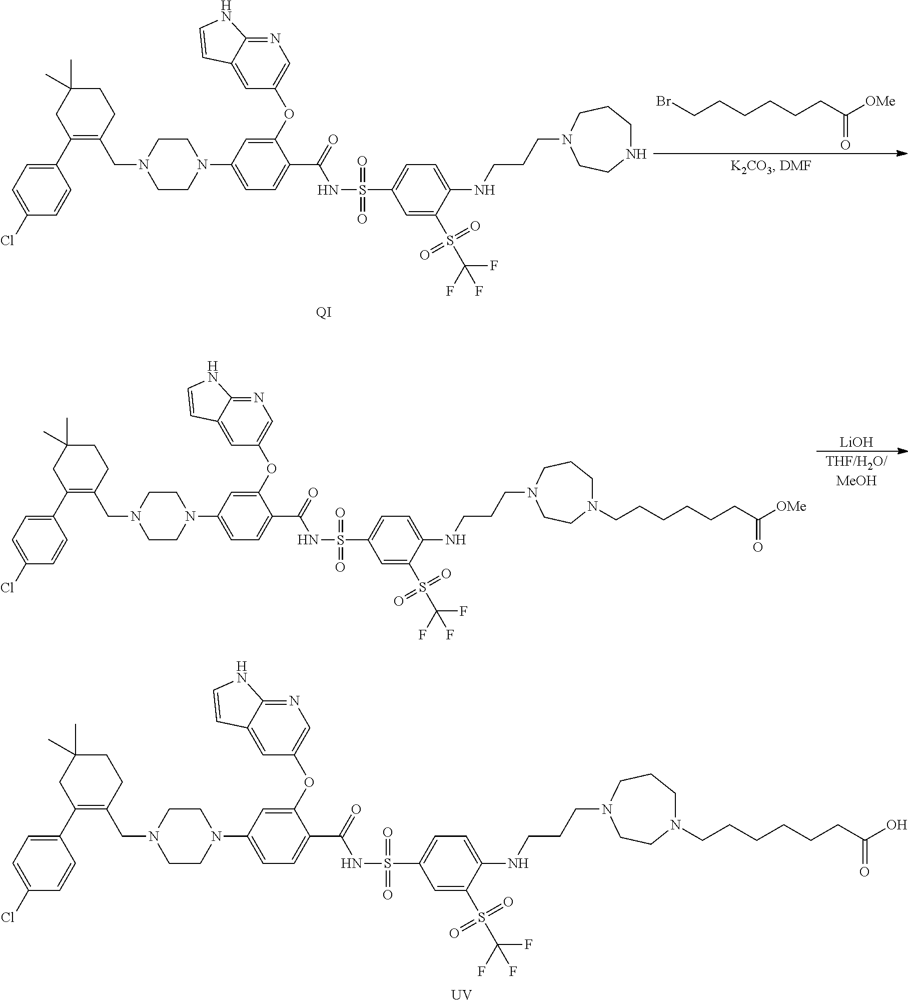

### Step 1—Methyl 7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)-1,4-diazepan-1-yl)heptanoate

To a solution of N-((4-((3-(1,4-diazepan-1-yl)propyl)amino)-3-((trifluoromethyl)sulfonyl)phenyl)sulfonyl)-2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzamide (530 mg, 531 μmol, Intermediate QI) and methyl 7-bromoheptanoate (237 mg, 1.06 mmol, CAS #54049-24-0) in DMF (4 mL) was added K₂CO₃ (220 mg, 1.59 mmol) and KI (8.82 mg, 53.1 μmol). The mixture was then stirred at 80° C. for 8 hrs. On completion, the reaction mixture was filtered to give the filtrate. The crude residue was purified by reversed-phase HPLC (0.1% FA condition) to give the title compound (320 mg) as a white solid. LC-MS (ESI⁺) m/z 570.9 (M+2H)⁺/2.

### Step 2—7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)-1,4-diazepan-1-yl)heptanoic Acid

To a solution of methyl 7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)-1,4-diazepan-1-yl)heptanoate (270 mg, 237 μmol) in THF (4 mL) and H₂O (2 mL) was added LiOH H₂O (49.7 mg, 1.18 mmol). The mixture was then stirred at 25° C. for 1 hr. On completion, HCl (1 mol) was added to the reaction mixture until the pH was 7, then the mixture was diluted with water (7 mL) and extracted with ethyl acetate (3×10 mL). The combined organic layers were dried over anhydrous sodium sulfate, filtered and the filtrate was concentrated in vacuo to the title compound (270 mg) as a white solid. LC-MS (ESI⁺) m/z 1125.6 (M+H)⁺.

### Synthesis of (2S,4R)-1-((S)-2-(1-fluorocyclopropane-1-carboxamido)-3,3-dimethylbutanoyl)-4-hydroxy-N—((S)-1-(2-hydroxy-4-(4-methylthiazol-5-yl)phenyl)ethyl)pyrrolidine-2-carboxamide (Intermediate UW)

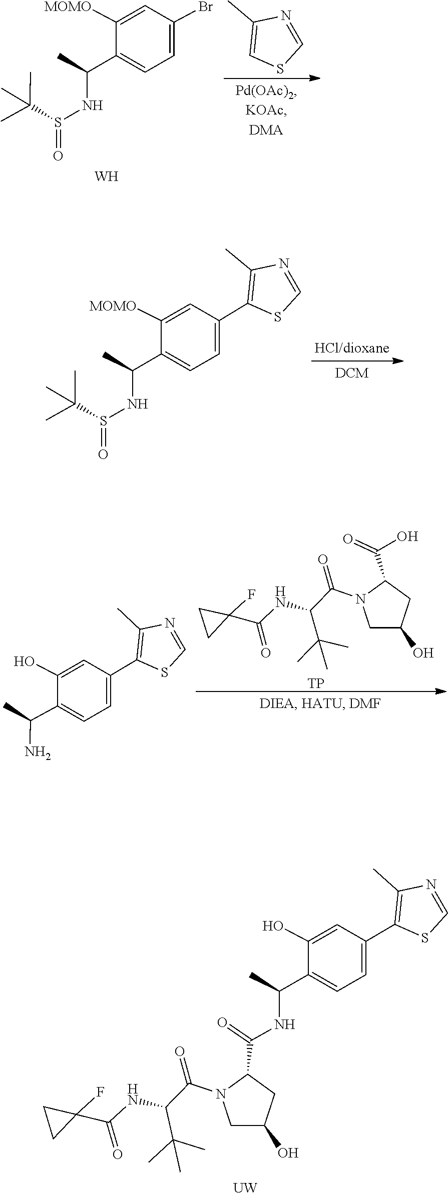

### Step 1—(R)—N—((S)-1-(2-(methoxymethoxy)-4-(4-methylthiazol-5-yl)phenyl)ethyl)-2-methylpropane-2-sulfinamide

A mixture of (R)—N-[(1S)-1-[4-bromo-2-(methoxymethoxy)phenyl]ethyl]-2-methyl-propane-2-sulfinamide (1.80 g, 4.94 mmol, Intermediate WH), 4-methylthiazole (980 mg, 9.88 mmol, CAS #693-95-8), Pd(OAc)₂ (111 mg, 494 μmol), and KOAc (970 mg, 9.88 mmol) in DMA (30 mL) was degassed and purged with N₂ three times, and then the mixture was stirred at 90° C. for 3 hrs under N₂ atmosphere. On completion, the reaction mixture was filtered, and the filtrate was quenched by addition of H₂O (20 mL), and extracted with ethyl acetate (20 mL×3). The combined organic layers were washed with brine (20 mL×3), dried over Na₂SO₄, filtered and concentrated under reduced pressure to give a residue. The residue was purified by column chromatography (SiO₂, Petroleum ether/Ethyl acetate=5/1 to 3/1) to give the title compound (1.10 g, 36% yield) as a yellow solid. LC-MS (ESI⁺) m/z 383.0 (M+H)⁺.

### Step 2—(S)-2-(1-aminoethyl)-5-(4-methylthiazol-5-yl)phenol

To a solution of (R)—N-[(1S)-1-[2-(methoxymethoxy)-4-(4-methylthiazol-5-yl)phenyl]ethyl]-2-methyl-propane-2-sulfinamide (1.00 g, 2.61 mmol) in DCM (33 mL) was added HCl/dioxane (4 M, 10 mL). The mixture was then stirred at 25° C. for 1 hr. On completion, the reaction mixture was concentrated under reduced pressure to remove DCM to give the title compound (740 mg) as a yellow solid. LC-MS (ESI⁺) m/z 235.1 (M+H)⁺.

### Step 3—(2S,4R)-1-((S)-2-(1-fluorocyclopropane-1-carboxamido)-3,3-dimethylbutanoyl)-4-hydroxy-N—((S)-1-(2-hydroxy-4-(4-methylthiazol-5-yl)phenyl)ethyl)pyrrolidine-2-carboxamide

To a solution of (2S,4R)-1-[(2S)-2-[(1-fluorocyclopropanecarbonyl)amino]-3,3-dimethyl-butanoyl]-4-hydroxy-pyrrolidine-2-carboxylic acid (1.02 g, 3.10 mmol, Intermediate TP) in DMF (20 mL) was added DIEA (1.67 g, 12.9 mmol, 2.25 mL) and HATU (1.28 g, 3.36 mmol). Then 2-[(1S)-1-aminoethyl]-5-(4-methylthiazol-5-yl)phenol (700 mg, 2.59 mmol, HCl) was added and the mixture was then stirred at 25° C. for 1 hr. On completion, the mixture was quenched by adding H₂O (2 mL) at 0° C., then extracted with DCM (2 mL×3). The organic phase was concentrated to give a crude product and purified by reversed-phase HPLC (0.8 g/L NH₄HCO₃ condition) to give the title compound (400 mg, 23% yield) as a white solid. LC-MS (ESI⁺) m/z 547.3 (M+H)⁺.

### Synthesis of 7-(2-((S)-1-((2S,4R)-1-((S)-2-(1-fluorocyclopropane-1-carboxamido)-3,3-dimethylbutanoyl)-4-hydroxypyrrolidine-2-carboxamido)ethyl)-5-(4-methylthiazol-5-yl)phenoxy)heptanoic Acid (Intermediate UX)

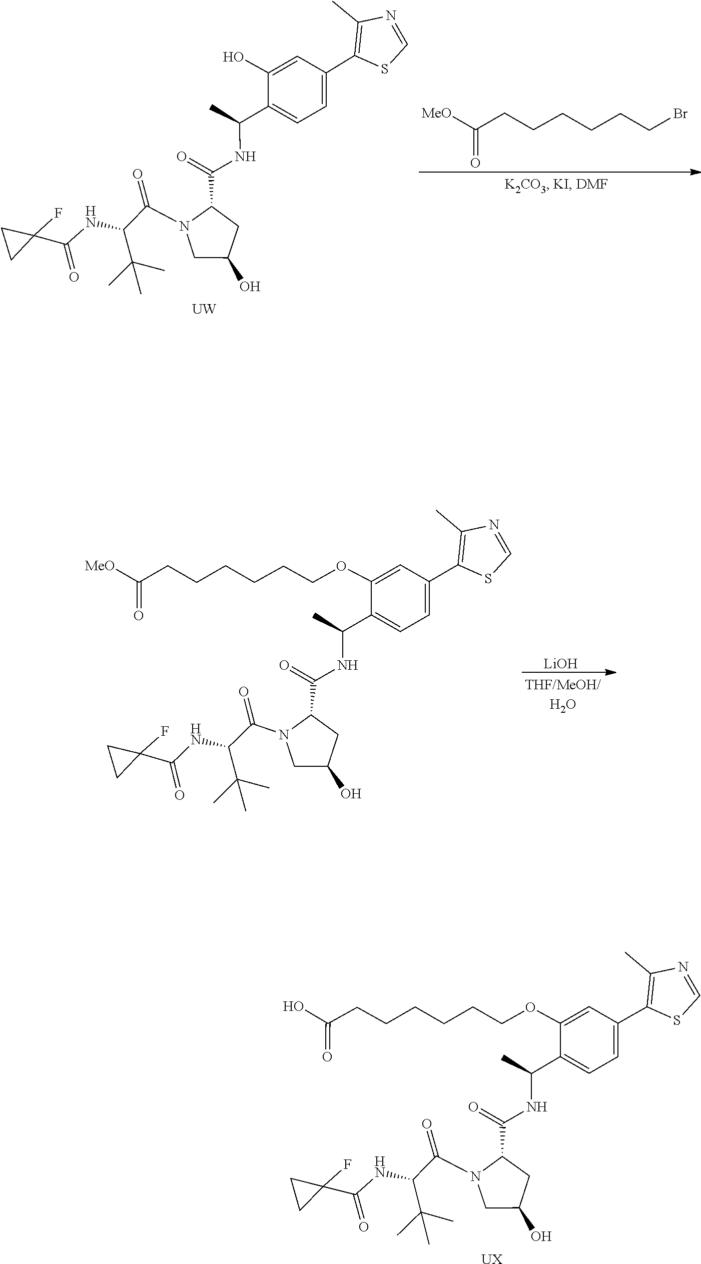

### Step 1—Methyl 7-(2-((S)-1-((2S,4R)-1-((S)-2-(1-fluorocyclopropane-1-carboxamido)-3,3-dimethylbutanoyl)-4-hydroxypyrrolidine-2-carboxamido)ethyl)-5-(4-methylthiazol-5-yl)phenoxy)heptanoate

To a solution of (2S,4R)-1-[(2S)-2-[(1-fluorocyclopropanecarbonyl)amino]-3,3-dimethyl-butanoyl]-4-hydroxy-N-[(1S)-1-[2-hydroxy-4-(4-methylthiazol-5-yl)phenyl]ethyl]pyrrolidine-2-carboxamide (250 mg, 457 μmol, Intermediate UW) in DMF (10 mL) was added K₂CO₃ (158 mg, 1.14 mmol), KI (75.9 mg, 457 μmol) and methyl 7-bromoheptanoate (122 mg, 549 μmol, CAS #54049-24-0). The mixture was then stirred at 70° C. for 12 hrs. On completion, the reaction mixture was quenched by addition of H₂O (10 mL), and then extracted with ethyl acetate (10 mL×3). The combined organic layers were washed with brine (10 mL×3), dried over Na₂SO₄, filtered and concentrated under reduced pressure to give a residue. The residue was purified by column chromatography (SiO₂, DCM:MeOH=15:1) to give the title compound (300 mg, 95% yield) as a yellow oil. LC-MS (ESI⁺) m/z 689.5 (M+H)⁺.

### Step 2—7-(2-((S)-1-((2S,4R)-1-((S)-2-(1-fluorocyclopropane-1-carboxamido)-3,3-dimethylbutanoyl)-4-hydroxypyrrolidine-2-carboxamido)ethyl)-5-(4-methylthiazol-5-yl)phenoxy)heptanoic Acid

To a solution of methyl 7-[2-[(1S)-1-[[(2S,4R)-1-[(2S)-2-[(1-fluorocyclopropanecarbonyl)amino]-3,3-dimethyl-butanoyl]-4-hydroxy-pyrrolidine-2-carbonyl]amino]ethyl]-5-(4-methylthiazol-5-yl)phenoxy]heptanoate (290 mg, 421 μmol) in THF (4 mL), MeOH (4 mL) and H₂O (2 mL) was added LiOH H₂O (53.0 mg, 1.26 mmol). The mixture was then stirred at 25° C. for 3 hrs. On completion, HCl (1N) was added to the reaction mixture until the pH=3.5, then the mixture was diluted with water (2 mL) and extracted with DCM/THF (3:1, 30 mL×3). The combined organic layers were dried over anhydrous sodium sulfate, filtered and the filtrate was concentrated in vacuo to give the title compound (280 mg) as a yellow solid. LC-MS (ESI⁺) m/z 675.3 (M+H)⁺.

### Synthesis of 2-((1H-pyrrolo[2,3-b]pyridin-5-yl)oxy)-4-(4-(((4R)-4-((5-(tert-butoxycarbonyl)-2,5-diazabicyclo[2.2.1]heptan-2-yl)methyl)-4′-chloro-4-methyl-3,4,5,6-tetrahydro-[1,1′-biphenyl]-2-yl)methyl)piperazin-1-yl)benzoic Acid (Intermediate UY)

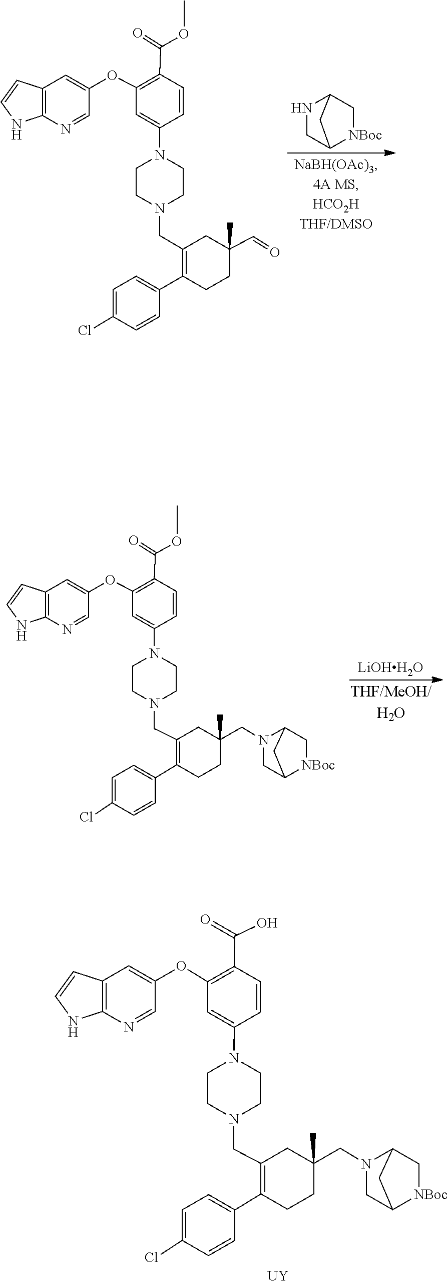 [SMILES: COC(=O)c1ccc(N2CCN(CC3=C(c4ccc(Cl)cc4)CC[C@@](C)(C=O)C3)CC2)cc1Oc1cnc2[nH]ccc2c1 ; CC(C)(C)OC(=O)N1CC2CC1CN2 ; COC(=O)c1ccc(N2CCN(CC3=C(c4ccc(Cl)cc4)CC[C@@](C)(CN4CC5CC4CN5C(=O)OC(C)(C)C)C3)CC2)cc1Oc1cnc2[nH]ccc2c1 ; "LiOH·H2O" ; CC(C)(C)OC(=O)N1CC2CC1CN2C[C@]1(C)CCC(c2ccc(Cl)cc2)=C(CN2CCN(c3ccc(C(=O)O)c(Oc4cnc5[nH]ccc5c4)c3)CC2)C1 ; "UY"]

### Step 1—Tert-butyl 5-(((R)-6-((4-(3-((1H-pyrrolo[2,3-b]pyridin-5-yl)oxy)-4-(methoxycarbonyl)phenyl)piperazin-1-yl)methyl)-4′-chloro-4-methyl-2,3,4,5-tetrahydro-[1,1′-biphenyl]-4-yl)methyl)-2,5-diazabicyclo[2.2.1]heptane-2-carboxylate

To a solution of methyl 4-[4-[[(5R)-2-(4-chlorophenyl)-5-formyl-5-methyl-cyclohexen-1-yl]methyl]piperazin-1-yl]-2-(1H-pyrrolo[2,3-b]pyridin-5-yloxy)benzoate (400 mg, 668 μmol synthesized via Steps 1-2 of Intermediate NU) in THF (10 mL) and DMSO (5 mL) was added tert-butyl 2,5-diazabicyclo[2.2.1]heptane-2-carboxylate (159 mg, 801 μmol, CAS #1217975-73-9), HCOOH (128 mg, 2.67 mmol) and 4 Å molecular sieves (800 mg, 668 μmol). The mixture was then stirred at 30° C. for 48 hrs. Next, NaBH(OAc)₃ (354 mg, 1.67 mmol) was added and the mixture was stirred at 30° C. for 2 hrs. On completion, the reaction mixture was filtered to give a filtrate. The filtrate was purified by prep-HPLC (0.1% FA condition) to give the title compound (300 mg, 54% yield) as a white solid. LC-MS (ESI⁺) m/z 781.4 (M+H)⁺.

### Step 2—2-((1H-pyrrolo[2,3-b]pyridin-5-yl)oxy)-4-(4-(((4R)-4-((5-(tert-butoxycarbonyl)-2,5-diazabicyclo[2.2.1]heptan-2-yl)methyl)-4′-chloro-4-methyl-3,4,5,6-tetrahydro-[1,1′-biphenyl]-2-yl)methyl)piperazin-1-yl)benzoic Acid

To a solution of tert-butyl 5-[[(1R)-4-(4-chlorophenyl)-3-[[4-[4-methoxycarbonyl-3-(1H-pyrrolo[2,3-b]pyridin-5-yloxy)phenyl]piperazin-1-yl]methyl]-1-methyl-cyclohex-3-en-1-yl]methyl]-2,5-diazabicyclo[2.2.1]heptane-2-carboxylate (300 mg, 363 μmol, FA) in H₂O (2 mL), THF (4 mL) and MeOH (4 mL) was added LiOH·H₂O (76.1 mg, 1.81 mmol). The mixture was then stirred at 50° C. for 12 hrs. On completion, the reaction mixture was quenched by water (10 mL) and extracted by DCM (30 mL). The combined organic layers were washed by brine (10 mL) and dried over anhydrous sodium sulfate, filtered and the filtrate was concentrated in vacuo to give the title compound as a light white solid (270 mg) LC-MS (ESI⁺) m/z 767.5 (M+H)⁺.

### Synthesis of 2-((1H-pyrrolo[2,3-b]pyridin-5-yl)oxy)-4-(4-(((4R)-4-((2,5-diazabicyclo[2.2.1]heptan-2-yl)methyl)-4′-chloro-4-methyl-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide (Intermediate UZ)

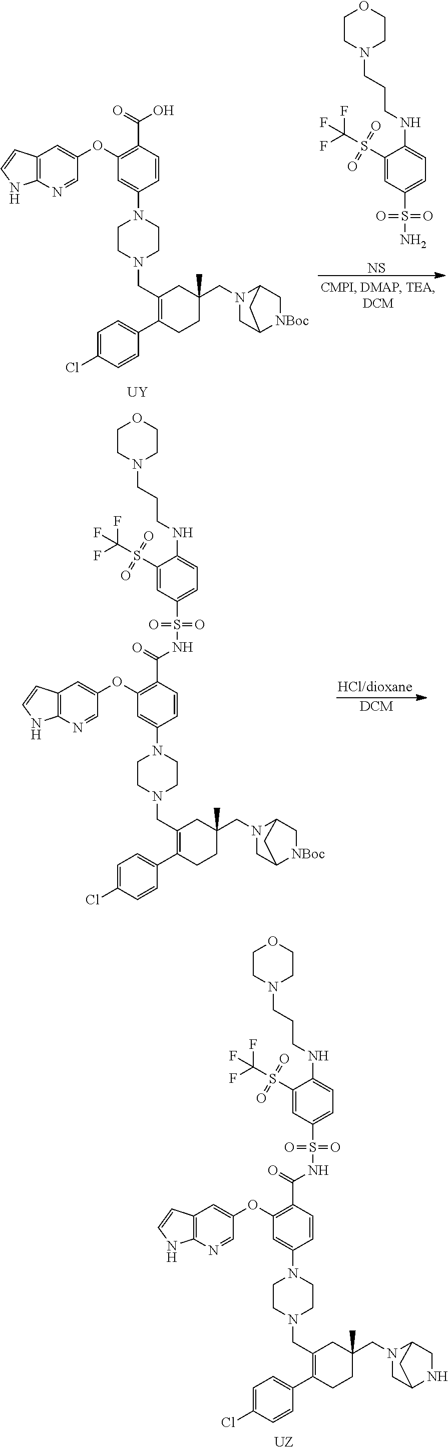

### Step 1—Tert-butyl 5-(((R)-6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)-2,5-diazabicyclo[2.2.1]heptane-2-carboxylate

To a solution of 4-[4-[[(5R)-5-[(5-tert-butoxycarbonyl-2,5-diazabicyclo[2.2.1]heptan-2-yl)methyl]-2-(4-chlorophenyl)-5-methyl-cyclohexen-1-yl]methyl]piperazin-1-yl]-2-(1H-pyrrolo[2,3-b]pyridin-5-yloxy)benzoic acid (240 mg, 313 μmol, Intermediate UY) in DCM (10 mL) was added DMAP (38.2 mg, 313 μmol), TEA (158 mg, 1.56 mmol) and CMPI (120 mg, 469 μmol). Then 4-(3-morpholinopropylamino)-3-(trifluoromethylsulfonyl)benzenesulfonamide (148 mg, 344 μmol, Intermediate NS) was added and the mixture was stirred at 25° C. for 12 hrs. On completion, the reaction mixture was quenched with water (10 mL) and extracted with DCM (30 mL). The combined organic layers were washed with brine (10 mL) and dried over anhydrous sodium sulfate, filtered and the filtrate was concentrated in vacuo to give the a residue. The crude product was purified by reversed-phase HPLC (0.1% NH₃H₂O condition) to give the title compound (222 mg, 60% yield) as a white solid. LC-MS (ESI⁺) m/z 1180.4 (M+H)⁺.

### Step 2—2-((1H-pyrrolo[2,3-b]pyridin-5-yl)oxy)-4-(4-(((4R)-4-((2,5-diazabicyclo[2.2.1]heptan-2-yl)methyl)-4′-chloro-4-methyl-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide

To a solution of tert-butyl 5-[[(1R)-4-(4-chlorophenyl)-1-methyl-3-[[4-[4-[[4-(3-morpholinopropylamino)-3-(trifluoromethylsulfonyl)phenyl]sulfonylcarbamoyl]-3-(1H-pyrrolo[2,3-b]pyridin-5-yloxy)phenyl]piperazin-1-yl]methyl]cyclohex-3-en-1-yl]methyl]-2,5-diazabicyclo[2.2.1]heptane-2-carboxylate (100 mg, 84.7 μmol) in DCM (4 mL) was added HCl/dioxane (4 M, 1 mL). The mixture was then stirred at 25° C. for 2 hrs. On completion, the reaction mixture was concentrated in vacuo to remove the solvent to give the title compound (90 mg) as a light yellow solid. LC-MS (ESI⁺) m/z 1080.4 (M+H)⁺.

### Synthesis of 10-(((S)-1-((2S,4R)-2-(((R)-1-(4-ethynylphenyl)-2-hydroxyethyl)carbamoyl)-4-hydroxypyrrolidin-1-yl)-3,3-dimethyl-1-oxobutan-2-yl)amino)-10-oxodecanoic Acid (Intermediate VA)

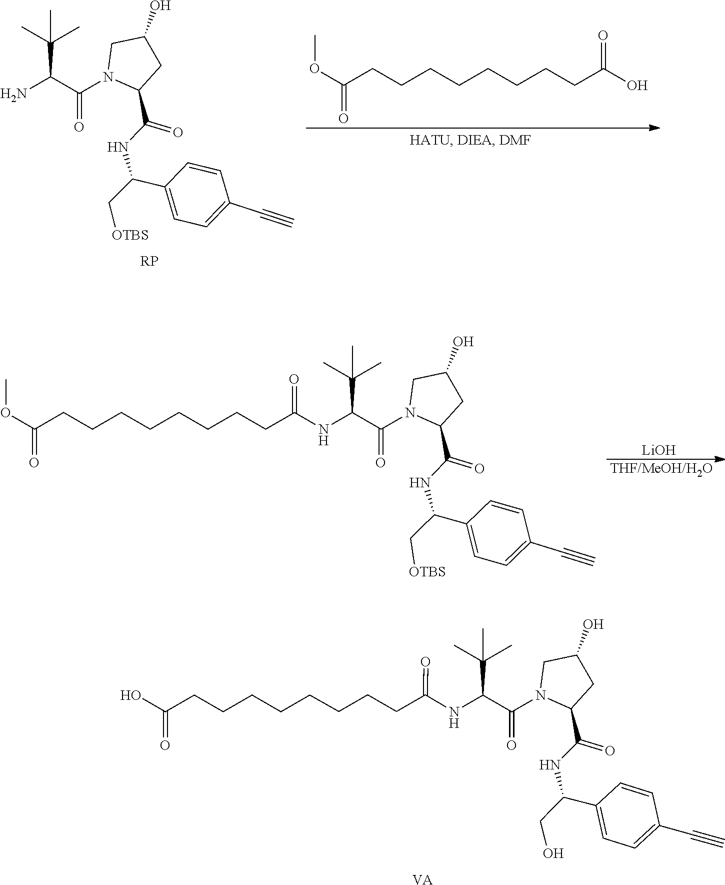

### Step 1—Methyl 10-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-10-oxodecanoate

To a solution of 10-methoxy-10-oxo-decanoic acid (108 mg, 498 μmol, CAS #818-88-2) in DMF (10 mL) was added DIEA (322 mg, 2.49 mmol, 434 uL) and HATU (284 mg, 747 μmol), then (2S,4R)-1-[(2S)-2-amino-3,3-dimethyl-butanoyl]-N-[(1R)-2-[tert-butyl(dimethyl)silyl]oxy-1-(4-ethynylphenyl)ethyl]-4-hydroxy-pyrrolidine-2-carboxamide (300 mg, 598 μmol, Intermediate RP) was added at 0° C. and the mixture was stirred at 0° C. for 1 hr. On completion, the reaction mixture was filtered to give a liquid. The liquid was purified by prep-HPLC (0.1% FA condition) to give the title compound (310 mg, 83% yield) as a white solid. LC-MS (ESI⁺) m/z 700.5 (M+H)⁺.

### Step 2—10-(((S)-1-((2S,4R)-2-(((R)-1-(4-ethynylphenyl)-2-hydroxyethyl)carbamoyl)-4-hydroxypyrrolidin-1-yl)-3,3-dimethyl-1-oxobutan-2-yl)amino)-10-oxodecanoic Acid

To a solution of methyl 10-[[(1S)-1-[(2S,4R)-2-[[(1R)-2-[tert-butyl(dimethyl)silyl]oxy-1-(4-ethynylphenyl)ethyl]carbamoyl]-4-hydroxy-pyrrolidine-1-carbonyl]-2,2-dimethyl-propyl]amino]-10-oxo-decanoate (310 mg, 416 μmol, FA) in H₂O (3 mL), MeOH (6 mL) and THF (6 mL) was added LiOH·H₂O (52.3 mg, 1.25 mmol). The mixture was then stirred at 25° C. for 2 hrs. On completion, the mixture was adjusted to pH of 6 by adding 1M HCl solution, then diluted with H₂O (10 mL) and extracted with DCM/THF (3:1, 30 mL×3). The combined organic layer was dried over anhydrous Na₂SO₄, filtered and the filtrate was concentrated to give a residue. The residue was purified by prep-HPLC (0.1% FA condition) to give the title compound (190 mg, 80% yield) as a white solid. LC-MS (ESI⁺) m/z 572.3 (M+H)⁺.

### Synthesis of (R)-2-((1H-pyrrolo[2,3-b]pyridin-5-yl)oxy)-4-(4-((4-(((tert-butoxycarbonyl)(methyl)amino)methyl)-4′-chloro-4-methyl-3,4,5,6-tetrahydro-[1,1′-biphenyl]-2-yl)methyl)piperazin-1-yl)benzoic Acid (Intermediate VB)

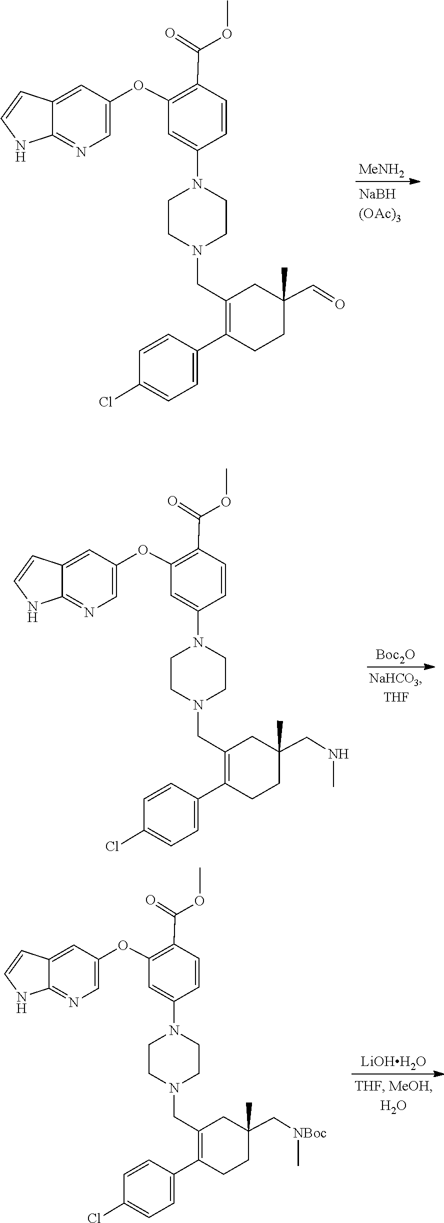

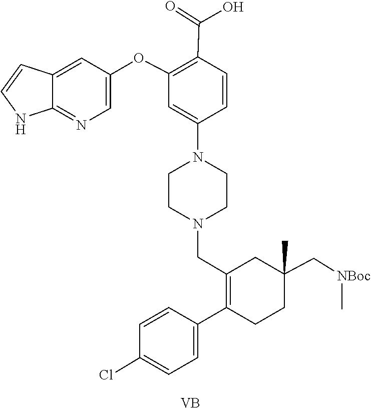

### Step 1—Methyl (R)-2-((1H-pyrrolo[2,3-b]pyridin-5-yl)oxy)-4-(4-((4′-chloro-4-methyl-4-((methylamino)methyl)-3,4,5,6-tetrahydro-[1,1′-biphenyl]-2-yl)methyl)piperazin-1-yl)benzoate

To a solution of methyl (R)-2-((1H-pyrrolo[2,3-b]pyridin-5-yl)oxy)-4-(4-((4′-chloro-4-formyl-4-methyl-3,4,5,6-tetrahydro-[1,1′-biphenyl]-2-yl)methyl)piperazin-1-yl)benzoate (1 g, 1.67 mmol, synthesized via Steps 1-2 of Intermediate NU) and MeNH₂ (156 mg, 5.01 mmol, HCl, CAS #593-51-1) in DCM (10 mL) was added AcOH (150 mg, 2.5 mmol), AcOK (491 mg, 5.01 mmol), and NaBH(OAc)₃ (460 mg, 2.17 mmol). The mixture was then stirred at 25° C. for 1 hr. On completion, the reaction mixture was diluted with water (30 mL) and extracted with dichloromethane (3×30 mL). The combined organic layers were washed with brine (90 mL) and dried over anhydrous sodium sulfate, filtered and the filtrate was concentrated in vacuo to give the title compound (800 mg) as a brown solid. LC-MS (ESI⁻) m/z 614.2 (M+H)⁺.

### Step 2—Methyl (R)-2-((1H-pyrrolo[2,3-b]pyridin-5-yl)oxy)-4-(4-((4-(((tert-butoxycarbonyl)(methyl)amino)methyl)-4′-chloro-4-methyl-3,4,5,6-tetrahydro-[1,1′-biphenyl]-2-yl)methyl)piperazin-1-yl)benzoate

To a solution of methyl (R)-2-((1H-pyrrolo[2,3-b]pyridin-5-yl)oxy)-4-(4-((4′-chloro-4-methyl-4-((methylamino)methyl)-3,4,5,6-tetrahydro-[1,1′-biphenyl]-2-yl)methyl)piperazin-1-yl)benzoate (700 mg, 1.14 mmol) in THF (10 mL) and NaHCO₃ (5 mL) was added (Boc)₂O (261 mg, 1.20 mmol). The mixture was then stirred at 25° C. for 1 hr. On completion, the reaction mixture was quenched by NH₄HCO₃ (20 mL) and extracted with ethyl acetate (3×20 mL). The combined organic layers were washed with brine (60 mL) and dried over anhydrous sodium sulfate, filtered and the filtrate was concentrated in vacuo to give the title compound (580 mg) as a yellow solid. LC-MS (ESI⁺) m/z 714.1 (M+H)⁺.

### Step 3—(R)-2-((1H-pyrrolo[2,3-b]pyridin-5-yl)oxy)-4-(4-((4-(((tert-butoxycarbonyl)(methyl)amino)methyl)-4′-chloro-4-methyl-3,4,5,6-tetrahydro-[1,1′-biphenyl]-2-yl)methyl)piperazin-1-yl)benzoic Acid

To a solution of methyl (R)-2-((1H-pyrrolo[2,3-b]pyridin-5-yl)oxy)-4-(4-((4-(((tert-butoxycarbonyl)(methyl)amino)methyl)-4′-chloro-4-methyl-3,4,5,6-tetrahydro-[1,1′-biphenyl]-2-yl)methyl)piperazin-1-yl)benzoate (580 mg, 812 μmol) in THF (2.5 mL). MeOH (2.5 mL) and H₂O (1 mL) was added LiOH·H₂O (170 mg, 4.06 mmol). The mixture was then stirred at 50° C. for 3 hrs. On completion, the reaction mixture was diluted with water (10 mL) and extracted with ethyl acetate (3×10 mL). The combined organic layers were washed with brine (30 mL) and dried over anhydrous sodium sulfate, filtered and the filtrate was concentrated in vacuo to give the title compound (550 mg) as a yellow solid. LC-MS (ESI⁺) m/z 700.5 (M+H)⁺.

### Synthesis of (R)-2-((1H-pyrrolo[2,3-b]pyridin-5-yl)oxy)-4-(4-((4′-chloro-4-methyl-4-((methylamino)methyl)-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide (Intermediate VC)

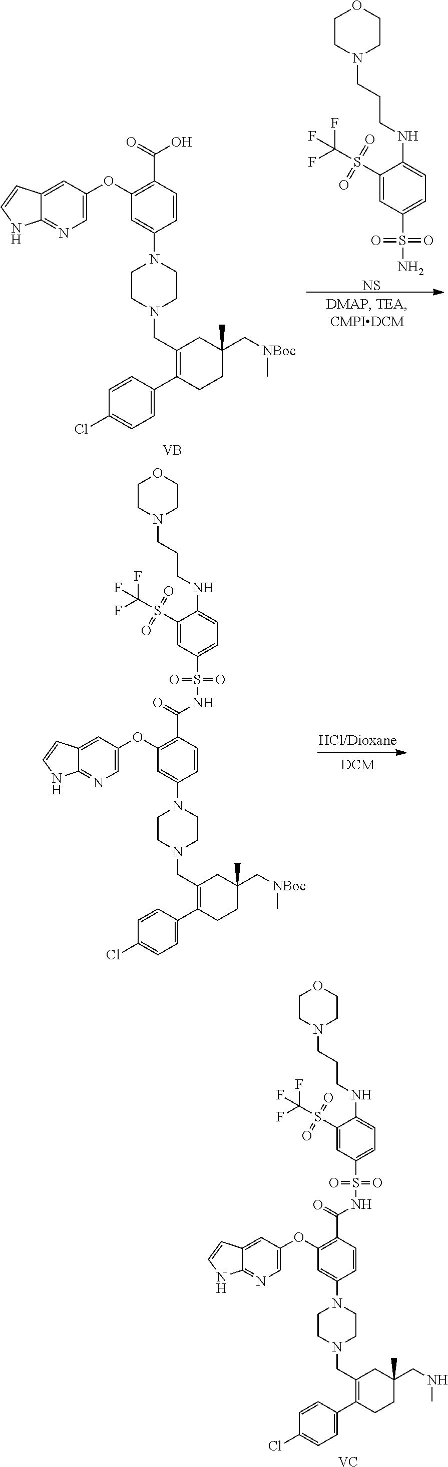

### Step 1—Tert-butyl ((S)-1-((2S,4R)-2-(((R)-1-(4-ethynylphenyl)-2-methoxyethyl)carbamoyl)-4-hydroxypyrrolidin-1-yl)-3,3-dimethyl-1-oxobutan-2-yl)carbamate

To a solution of (R)-2-((1H-pyrrolo[2,3-b]pyridin-5-yl)oxy)-4-(4-((4-(((tert-butoxycarbonyl)(methyl)amino)methyl)-4′-chloro-4-methyl-3,4,5,6-tetrahydro-[1,1′-biphenyl]-2-yl)methyl)piperazin-1-yl)benzoic acid (500 mg, 714 μmol, Intermediate VB) in DCM (10 mL) was added DMAP (87.2 mg, 714 μmol), TEA (217 mg, 2.14 mmol), CMPI (456.1 mg, 1.79 mmol) and 4-((3-morpholinopropyl)amino)-3-((trifluoromethyl)sulfonyl)benzenesulfonamide (339 mg, 785 μmol, Intermediate NS). The mixture was then stirred at 25° C. for 1 hr. On completion, the crude product was purified by reversed-phase HPLC (0.1% FA condition) to give the title compound (335 mg, 39% yield) as a yellow solid. LC-MS (ESI⁺) m/z 1113.8 (M+H)⁺.

### Step 2—(R)-2-((1H-pyrrolo[2,3-b]pyridin-5-yl)oxy)-4-(4-((4′-chloro-4-methyl-4-((methylamino)methyl)-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide

To a solution of tert-butyl ((S)-1-((2S,4R)-2-(((R)-1-(4-ethynylphenyl)-2-methoxyethyl)carbamoyl)-4-hydroxypyrrolidin-1-yl)-3,3-dimethyl-1-oxobutan-2-yl)carbamate (335 mg, 301 μmol) in DCM (4 mL) was added HCl/dioxane (4 M, 1 mL) at 25° C., then the reaction was stirred at 25° C. for 1 hr. On completion, the reaction mixture was concentrated in vacuo to give the title compound (300 mg) as a white solid. LC-MS (ESI⁺) m/z 1013.4 (M+H)⁺.

### Synthesis of (R)-7-(((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)(methyl)amino)heptanoic Acid (Intermediate VD)

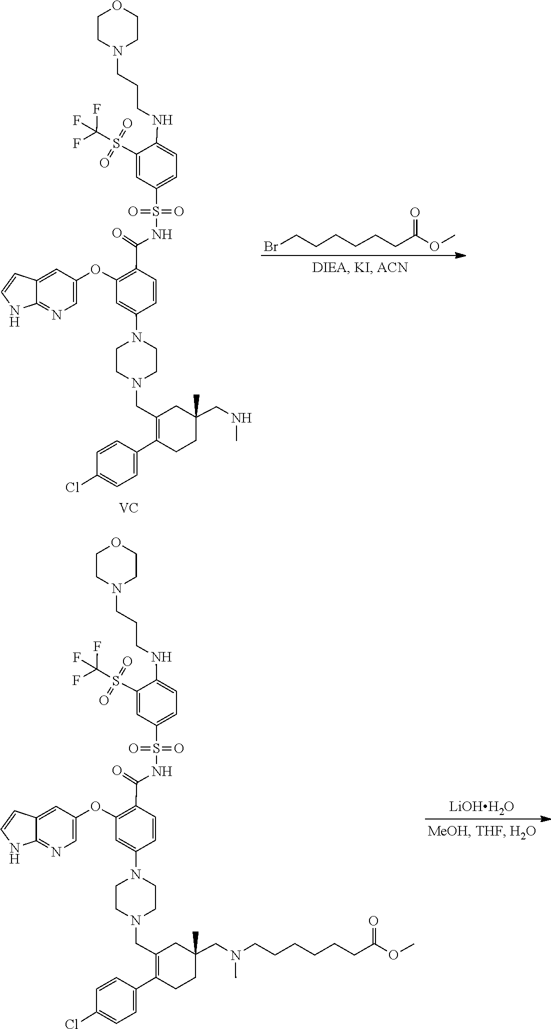

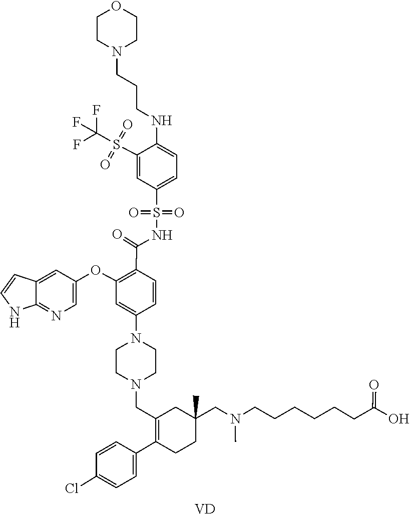 [SMILES: CN(CCCCCCC(=O)O)C[C@]1(C)CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1 ; "VD"]

### Step 1—Methyl (R)-7-(((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)(methyl)amino)heptanoate

To a solution of (R)-2-((1H-pyrrolo[2,3-b]pyridin-5-yl)oxy)-4-(4-((4′-chloro-4-methyl-4-((methylamino)methyl)-3,4,5,6-tetrahydro-[1,1′-biphenyl]-2-yl)methyl)piperazin-1-yl)-N-((4-((3-morpholinopropyl)amino)-3-((trifluoromethyl)sulfonyl)phenyl)sulfonyl)benzamide (200 mg, 197 μmol, Intermediate VC) and methyl 7-bromoheptanoate (176 mg, 789 μmol, CAS #54049-24-0) in ACN (2 mL) was added DIEA (255 mg, 1.97 mmol) and KI (32.8 mg, 197 μmol). The mixture was then stirred at 80° C. for 36 hrs. On completion, the crude product was purified by reversed-phase HPLC (0.8 g/L ammonium bicarbonate) to give the title compound (100 mg, 44% yield) as a white solid. LC-MS (ESI⁺) m/z 1155.9 (M+H)⁺.

### Step 2—(R)-7-(((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)(methyl)amino)heptanoic Acid

To a solution of methyl (R)-7-(((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)(methyl)amino)heptanoate (100 mg, 86.5 μmol) in THF (1 mL), MeOH (1 mL), and H₂O (0.4 mL) was added LiOH·H₂O (18.2 mg, 433 μmol). The mixture was then stirred at 40° C. for 1 hr. On completion, the reaction mixture was diluted with water (5 mL) and extracted with dichloromethane (3×5 mL). The combined organic layers were washed with brine (15 mL) and dried over anhydrous sodium sulfate, filtered and the filtrate was concentrated in vacuo to give the title compound (60 mg) as a yellow solid. LC-MS (ESI⁺) m/z 1141.8 (M+H)⁺.

### Synthesis of Tert-butyl 4-(3-(4-sulfamoyl-2-((trifluoromethyl)sulfonyl)phenoxy)propyl)piperazine-1-carboxylate (Intermediate VE)

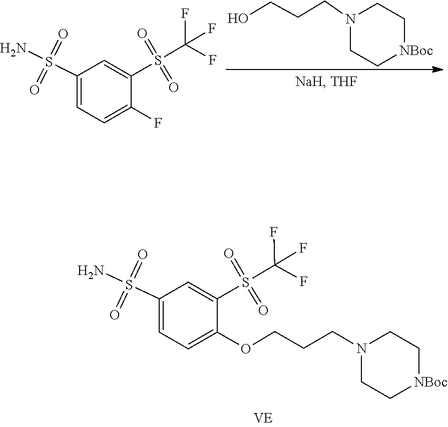

To a solution of tert-butyl 4-(3-hydroxypropyl)piperazine-1-carboxylate (795 mg, 3.25 mmol, CAS #132710-90-8) in THF (10.0 mL) was added NaH (130 mg, 3.25 mmol, 60% dispersion in mineral oil) at 0° C. and then 4-fluoro-3-((trifluoromethyl)sulfonyl)benzenesulfonamide (1.00 g, 3.25 mmol, CAS #1027345-08-9) was added, and the mixture was stirred at 25° C. for 2 hrs. The reaction mixture was diluted with water (5 mL) and extracted with ethyl acetate (2 mL×3), dried over Na₂SO₄, filtered and concentrated under reduced pressure to give a residue (1.5 g) as a white solid. ¹H NMR (400 MHz, DMSO-d₆) δ=8.48 (ddd, J=2.4, 4.4, 8.8 Hz, 1H), 8.43 (dd, J=2.4, 6.4 Hz, 1H), 7.98 (dd, J=8.8, 10.4 Hz, 1H), 7.81 (s, 2H), 4.03 (q, J=7.2 Hz, 1H), 3.43 (t, J=6.4 Hz, 2H), 3.32-3.25 (m, 4H), 2.36-2.33 (m, 1H), 2.33-2.27 (m, 4H), 2.00-1.98 (m, 1H), 1.62-1.52 (m, 2H), 1.39 (s, 8H), 1.20-1.15 (m, 1H).

### Synthesis of 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(3-(piperazin-1-yl)propoxy)-3-((trifluoromethyl)sulfonyl)phenyl)sulfonyl)benzamide (Intermediate VF)

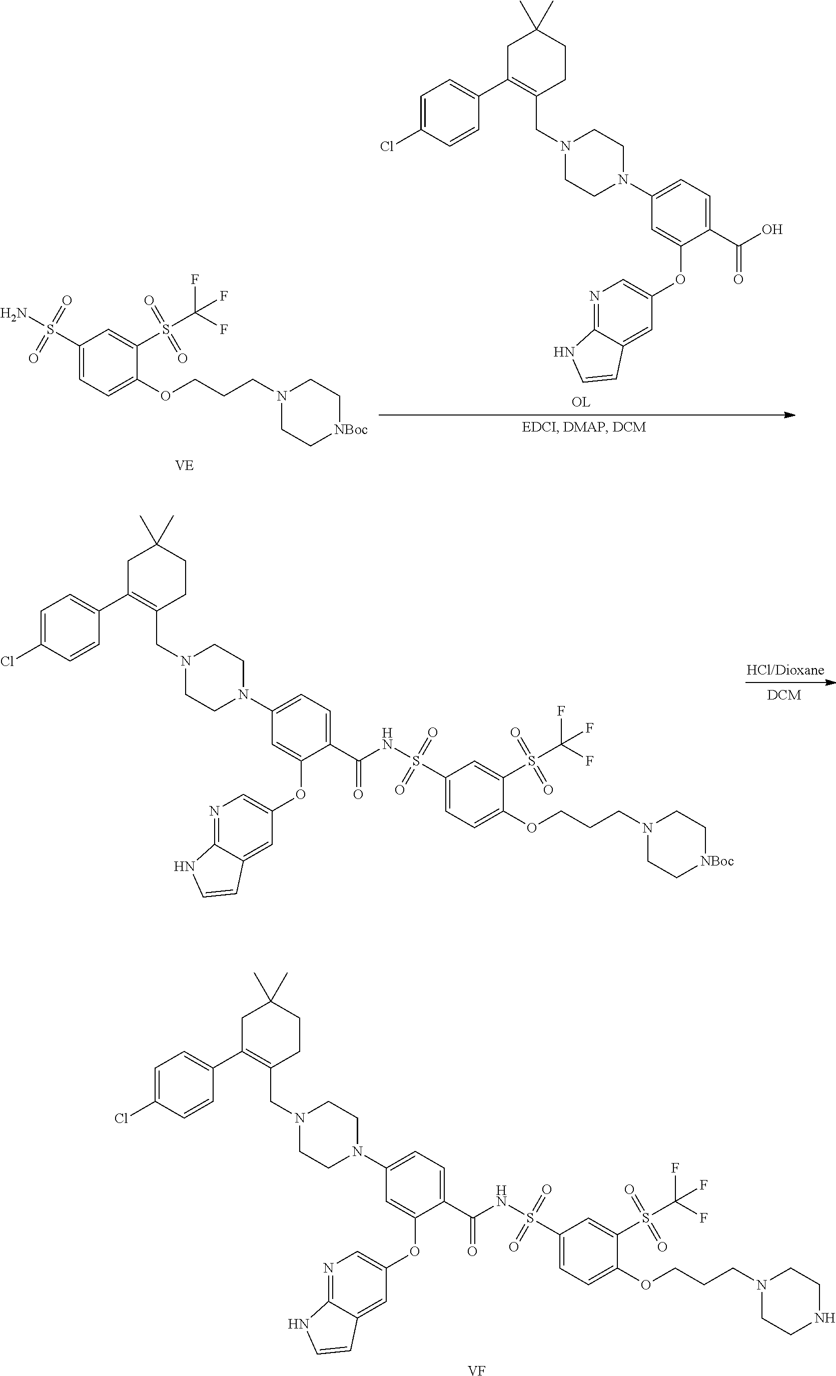 [SMILES: CC(C)(C)OC(=O)N1CCN(CCCOc2ccc(S(N)(=O)=O)cc2S(=O)(=O)C(F)(F)F)CC1 ; CC1(C)CCC(CN2CCN(c3ccc(C(=O)O)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(OCCCN5CCN(C(=O)OC(C)(C)C)CC5)c(S(=O)(=O)C(F)(F)F)c4)c(Oc4cnc5[nH]ccc5c4)c3)CC2)=C(c2ccc(Cl)cc2)C1 ; CC1(C)CCC(CN2CCN(c3ccc(C(=O)NS(=O)(=O)c4ccc(OCCCN5CCNCC5)c(S(=O)(=O)C(F)(F)F)c4)c(Oc4cnc5[nH]ccc5c4)c3)CC2)=C(c2ccc(Cl)cc2)C1 ; "VF"]

### Step 1—Tert-butyl 4-(3-(4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenoxy)propyl)piperazine-1-carboxylate

To a solution of tert-butyl 4-(3-(4-sulfamoyl-2-((trifluoromethyl)sulfonyl)phenoxy)propyl)piperazine-1-carboxylate (234 mg, 440 μmol, Intermediate VE) and 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoic acid (251 mg, 440 μmol, Intermediate OL) in DCM (5 mL) was added DMAP (107 mg, 880 μmol) and EDC (136 mg, 880 μmol). The mixture was then stirred at 25° C. for 2 hrs. On completion, the reaction mixture was concentrated under reduced pressure to give the title compound (320 mg) as a light yellow solid. LC-MS (ESI⁺) m/z 543.1 (M+H)⁺.

### Step 2—2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((4-(3-(piperazin-1-yl)propoxy)-3-((trifluoromethyl)sulfonyl)phenyl)sulfonyl)benzamide

To a solution of tert-butyl tert-butyl 4-(3-(4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenoxy)propyl)piperazine-1-carboxylate (320 mg, 295 μmol) in DCM (7.5 mL) was added HCl/dioxane (4 M, 1.20 mL). The mixture was then stirred at 25° C. for 0.5 hrs. On completion, the reaction mixture was concentrated under reduced pressure to remove dichloromethane. The crude residue was purified by reversed-phase HPLC (0.8 g/L, 0.1% FA condition) to give the title compound (57 mg) as a light pink solid. LC-MS (ESI⁺) m/z 493.2 (M+H)⁺; ¹H NMR (400 MHz, DMSO-d₆) δ=11.53-11.51 (m, 1H), 8.23 (d, J=2.4 Hz, 1H), 8.19 (s, 1H), 7.95 (d, J=2.4 Hz, 1H), 7.91 (d, J=2.4 Hz, 1H), 7.60 (d, J=8.4 Hz, 1H), 7.44-7.41 (m, 1H), 7.36 (d, J=8.4 Hz, 2H), 7.24 (d, J=2.8 Hz, 1H), 7.12 (d, J=9.6 Hz, 1H), 7.06 (d, J=8.4 Hz, 2H), 6.63 (d, J=8.4 Hz, 1H), 6.32-6.26 (m. 2H), 4.24-4.20 (m, 2H), 3.04-2.97 (m, 10H), 2.74 (s, 2H), 2.68 (s, 1H), 2.34 (s, 2H), 2.24-2.14 (m, 8H), 1.96 (s, 2H), 1.92-1.86 (m, 2H), 1.42-1.37 (m, 2H), 0.94 (s, 7H).

### Synthesis of 9-(((S)-1-((2S,4R)-2-(((R)-1-(4-ethynylphenyl)-2-hydroxyethyl)carbamoyl)-4-hydroxypyrrolidin-1-yl)-3,3-dimethyl-1-oxobutan-2-yl)amino)-9-oxononanoic Acid (Intermediate VG)

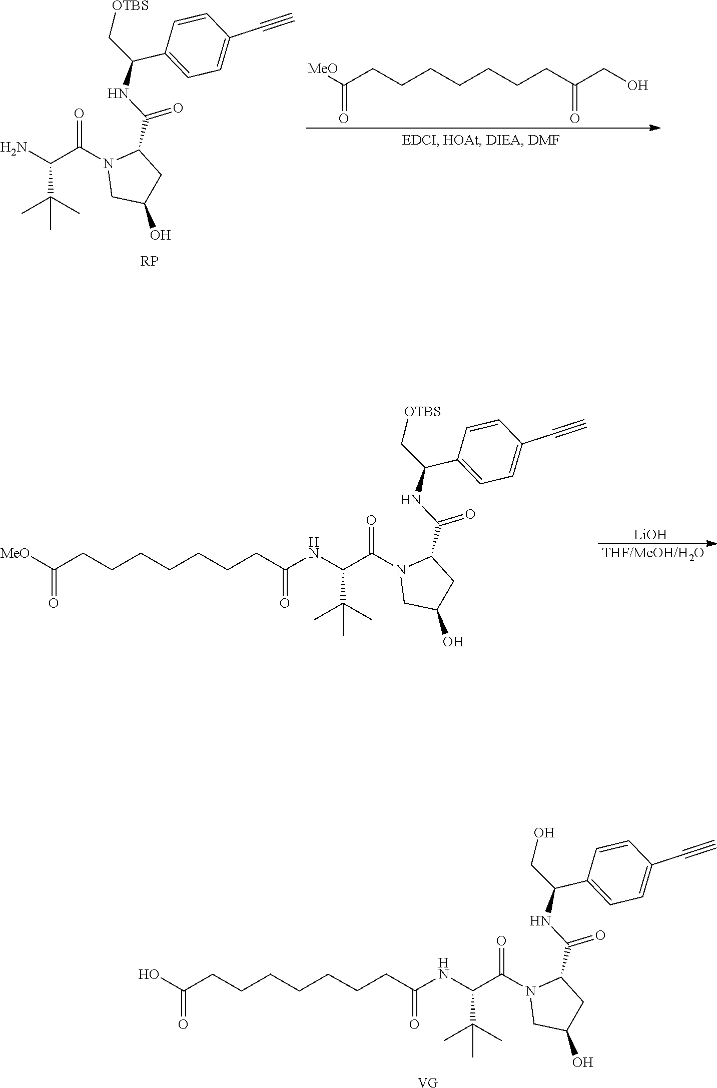

### Step 1—Methyl 9-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-9-oxononanoate

To a solution of (2S,4R)-1-((S)-2-amino-3,3-dimethylbutanoyl)-N—((R)-2-((tert-butyldimethylsilyl)oxy)-1-(4-ethynylphenyl)ethyl)-4-hydroxypyrrolidine-2-carboxamide (300 mg, 597 μmol, Intermediate RP) in DMF (4 mL) was added 9-methoxy-9-oxononanoic acid (157 mg, 777 μmol, CAS #2104-19-0), DIEA (772 mg, 5.98 mmol, 1.04 mL), HOAt (122 mg, 896 μmol, 125 uL) and EDCI (171 mg, 896 μmol). The mixture was then stirred at 25° C. for 12 hrs. On completion, the mixture was filtered to give a crude product. The crude product was purified by reversed-phase HPLC (0.1% FA condition) to give the title compound (330 mg, 80% yield) as a white solid. LC-MS (ESI⁺) m/z 686.6. (M+H)⁺.

### Step 2—9-(((S)-1-((2S,4R)-2-(((R)-1-(4-ethynylphenyl)-2-hydroxyethyl) carbamoyl)-4-hydroxypyrrolidin-1-yl)-3,3-dimethyl-1-oxobutan-2-yl)amino)-9-oxononanoic Acid

To a solution of methyl 9-(((S)-1-((2S,4R)-2-(((R)-2-((tert-butyldimethylsilyl)oxy)-1-(4-ethynylphenyl)ethyl)carbamoyl)-4-hydroxypyrrolidin-1-yl)-3,3-dimethyl-1-oxobutan-2-yl)amino)-9-oxononanoate (330 mg, 481 μmol) in THF (8 mL), MeOH (8 mL) and H₂O (5 mL) was added LiOH·H₂O (100 mg, 2.41 mmol). The mixture was then stirred at 25° C. for 2 hrs. On completion, the mixture was adjusted to pH to 3 with HCl (1 M), then extracted with DCM/THF (3:1, 3×20 mL), and concentrated to give the crude product. The crude product was purified by reversed-phase HPLC (0.1% HCl condition) to give the title compound (220 mg, 73% yield, HCl) as a white solid. LC-MS (ESI⁺) m/z 642.3. (M+H)⁺.

### Synthesis of (R)-1-(4-ethynylphenyl)-2-methoxyethan-1-amine (Intermediate VH)

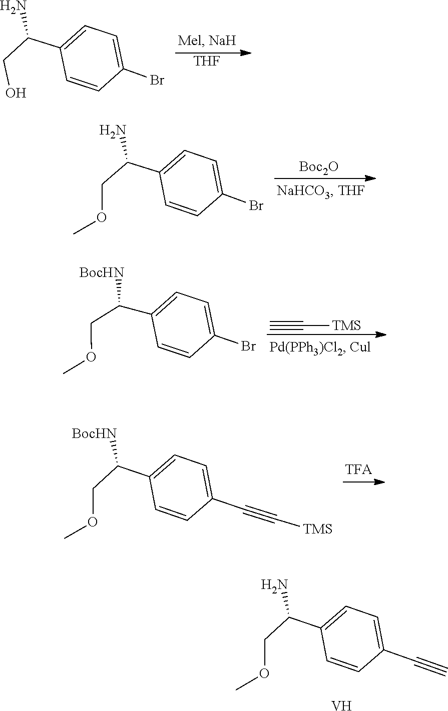

### Step 1—(R)-1-(4-bromophenyl)-2-methoxyethan-1-amine

To a solution of (R)-2-amino-2-(4-bromophenyl)ethan-1-ol (2 g, 9.3 mmol, CAS #354153-64-3) in THF (60 mL) was added NaH (740 mg, 18.5 mmol, 60% dispersion in mineral oil). After stirring for 30 mins at rt, MeI (1.38 g, 9.72 mmol) was added slowly, then the mixture was stirred at 25° C. for 2 hrs. On completion, the reaction mixture was quenched with NH₄Cl (50 mL) and extracted with ethyl acetate (3×50 mL). The combined organic layers were washed with brine (150 mL), dried over anhydrous sodium sulfate, filtered and the filtrate was concentrated in vacuo to give the crude residue. Then the crude residue was purified by reversed-phase HPLC (0.8 g/L ammonium bicarbonate) to give the title compound (1.4 g, 66% yield) as a yellow solid. LC-MS (ESI⁺) m/z 214.8 (M+H-16)⁺.

### Step 2—Tert-butyl (R)-(1-(4-bromophenyl)-2-methoxyethyl)carbamate

To a solution of (R)-1-(4-bromophenyl)-2-methoxyethan-1-amine (1.2 g, 5.2 mmol) in NaHCO₃ (5 mL) and THF (10 mL) was added (Boc)₂O (1.20 g, 5.48 mmol). The mixture was then stirred at 25° C. for 1 hr. On completion, the reaction mixture was quenched with NH₄HCO₃ (20 mL) and extracted with ethyl acetate (3×20 mL). The combined organic layers were washed with brine (40 mL), dried over anhydrous sodium sulfate, filtered and the filtrate was concentrated in vacuo to give the title compound (1.4 g) as a yellow oil. LC-MS (ESI+) m/z 274.1 (M+H-56)⁺.

### Step 3—Tert-butyl (R)-(2-methoxy-1-(4-((trimethylsilyl)ethynyl)phenyl)ethyl)carbamate

To a solution of tert-butyl (R)-(1-(4-bromophenyl)-2-methoxyethyl)carbamate (1.2 g, 3.63 mmol) in TEA (12 mL) was added Pd(PPh₃)₂Cl₂ (510 mg, 727 μmol), CuI (138 mg, 727 μmol) and ethynyltrimethylsilane (5.35 g, 54.5 mmol, CAS #1066-54-2). The mixture was stirred at 80° C. for 12 hrs. On completion, the crude product was purified by reversed-phase HPLC (0.8 g/L ammonium bicarbonate) to give the title compound (2 g, 86% yield) as a yellow oil. LC-MS (ESI⁺) m/z 231.2 (M+H−100)⁺.

### Step 4—(R)-1-(4-ethynylphenyl)-2-methoxyethan-1-amine

To a solution of tert-butyl (R)-(2-methoxy-1-(4-((trimethylsilyl)ethynyl)phenyl)ethyl)carbamate (1.1 g, 3.17 mmol) in DCM (22 mL) was added TFA (8.44 g, 74.1 mmol). The mixture was then stirred at 25° C. for 10 mins. On completion, the reaction mixture was diluted with water (40 mL) and extracted with ethyl acetate (3×40 mL). The combined organic layers were washed with brine (120 mL) and dried over anhydrous sodium sulfate, filtered and the filtrate was concentrated in vacuo to give the title compound (700 mg) as a yellow oil. LC-MS (ESI⁺) m/z 159.2 (M+H−16)⁺.

### Synthesis of (2S,4R)-1-((S)-2-amino-3,3-dimethylbutanoyl)-N—((R)-1-(4-ethynylphenyl)-2-methoxyethyl)-4-hydroxypyrrolidine-2-carboxamide (Intermediate VI)

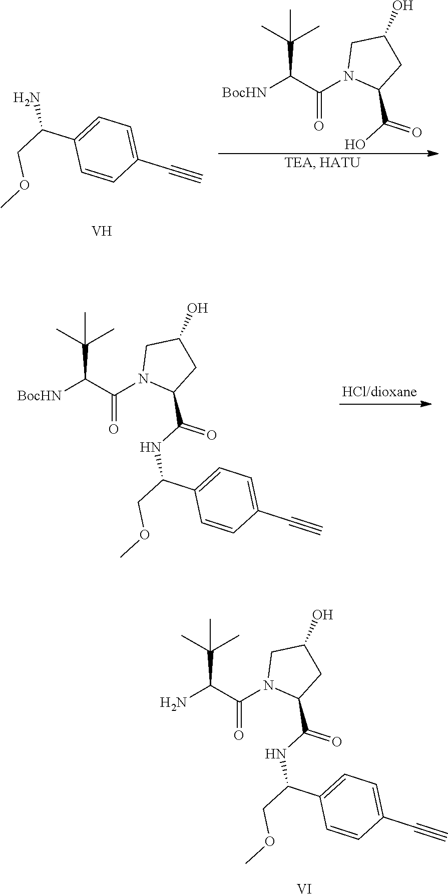

### Step 1—Tert-butyl ((S)-1-((2S,4R)-2-(((R)-1-(4-ethynylphenyl)-2-methoxyethyl)carbamoyl)-4-hydroxypyrrolidin-1-yl)-3,3-dimethyl-1-oxobutan-2-yl)carbamate

To a solution of (R)-1-(4-ethynylphenyl)-2-methoxyethan-1-amine (600 mg, 3.42 mmol, Intermediate VH) in DCM (12 mL) was added TEA (1.73 g, 17.1 mmol), HATU (1.56 g, 4.11 mmol) and (2S,4R)-1-((S)-2-((tert-butoxycarbonyl)amino)-3,3-dimethylbutanoyl)-4-hydroxypyrrolidine-2-carboxylic acid (1.18 g, 3.42 mmol) at 0° C. and the mixture was stirred at 0° C. for 10 mins. On completion, the reaction mixture was diluted with water (30 mL) and extracted with ethyl acetate (3×30 mL). The combined organic layers were washed with brine (90 mL), dried over anhydrous sodium sulfate, filtered and the filtrate was concentrated in vacuo to give the crude residue. Then the residue was purified by column chromatography (SiO2, Petroleum ether/Ethyl acetate=8/1 to 5/1) to give the title compound (1.8 g, 73% yield) as a yellow oil. LC-MS (ESI⁺) m/z 502.3 (M+H)⁺.

### Step 2—(2S,4R)-1-((S)-2-amino-3,3-dimethylbutanoyl)-N—((R)-1-(4-ethynylphenyl)-2-methoxyethyl)-4-hydroxypyrrolidine-2-carboxamide

To a solution of tert-butyl ((S)-1-((2S,4R)-2-(((R)-1-(4-ethynylphenyl)-2-methoxyethyl)carbamoyl)-4-hydroxypyrrolidin-1-yl)-3,3-dimethyl-1-oxobutan-2-yl)carbamate (400 mg, 797 μmol) in DCM (4 mL) was added HCl/dioxane (4 M, 1 mL). The mixture was then stirred at 25° C. for 1 hr. On completion, the reaction mixture concentrated in vacuo to give the title compound (320 mg) as a yellow solid. LC-MS (ESI⁺) m/z 402.3 (M+H)⁺.

### Synthesis of 8-(((S)-1-((2S,4R)-2-(((R)-1-(4-ethynylphenyl)-2-methoxyethyl)carbamoyl)-4-hydroxypyrrolidin-1-yl)-3,3-dimethyl-1-oxobutan-2-yl)amino)-8-oxooctanoic Acid (Intermediate VJ)

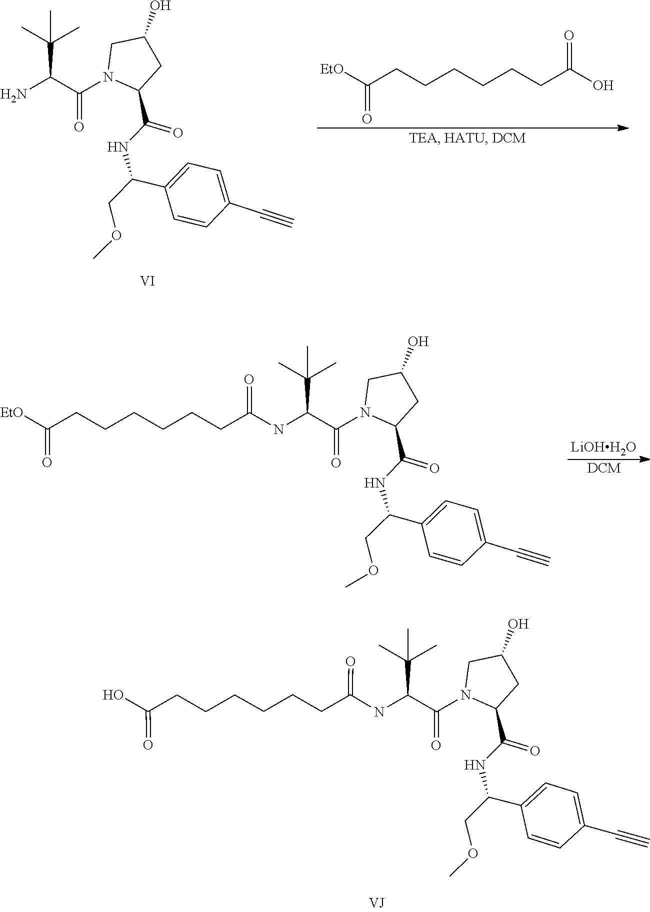

### Step 1—Ethyl 8-(((S)-1-((2S,4R)-2-(((R)-1-(4-ethynylphenyl)-2-methoxyethyl)carbamoyl)-4-hydroxypyrrolidin-1-yl)-3,3-dimethyl-1-oxobutan-2-yl)amino)-8-oxooctanoate

To a solution of 8-ethoxy-8-oxooctanoic acid (165 mg, 877 μmol, CAS #3946-32-5) in DCM (4 mL) was added TEA (403 mg, 3.99 mmol), HATU (364 mg, 956 μmol) and (2S,4R)-1-((S)-2-amino-3,3-dimethylbutanoyl)-N—((R)-1-(4-ethynylphenyl)-2-methoxyethyl)-4-hydroxypyrrolidine-2-carboxamide (320 mg, 797 μmol, Intermediate VI). The mixture was then stirred at 0° C. for 10 mins. On completion, crude product was purified by reversed-phase HPLC (0.1% FA condition) to give the title compound (150 mg, 32% yield) as a white solid. LC-MS (ESI⁺) m/z 572.5 (M+H)⁺.

### Step 2—8-(((S)-1-((2S,4R)-2-(((R)-1-(4-ethynylphenyl)-2-methoxyethyl)carbamoyl)-4-hydroxypyrrolidin-1-yl)-3,3-dimethyl-1-oxobutan-2-yl)amino)-8-oxooctanoic Acid

To a solution of ethyl 8-(((S)-1-((2S,4R)-2-(((R)-1-(4-ethynylphenyl)-2-methoxyethyl)carbamoyl)-4-hydroxypyrrolidin-1-yl)-3,3-dimethyl-1-oxobutan-2-yl)amino)-8-oxooctanoate (150 mg, 262 μmol) in THF (1 mL), MeOH (1 mL) and H₂O (0.4 mL) was added LiOH·H₂O (55.1 mg, 1.31 mmol). The mixture was then stirred at 40° C. for 1 hr. On completion, the reaction mixture was diluted with water (5 mL) and extracted with ethyl acetate (3×5 mL). The combined organic layers were washed with brine (15 mL)), dried over anhydrous sodium sulfate, filtered and the filtrate was concentrated in vacuo to give the title compound (140 mg) as a white solid. LC-MS (ESI⁺) m/z 558.5 (M+H)⁺.

### Synthesis of (2S,4R)-1-((S)-2-amino-3,3-dimethylbutanoyl)-N—((S)-1-(4-ethynyl-2-hydroxyphenyl)ethyl)-4-hydroxypyrrolidine-2-carboxamide (Intermediate VK)

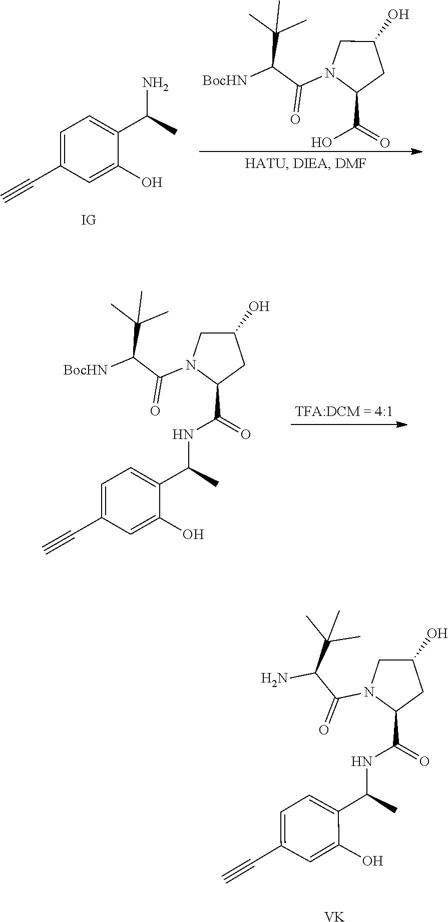

### Step 1—Tert-butyl ((S)-1-((2S,4R)-2-(((S)-1-(4-ethynyl-2-hydroxyphenyl)ethyl)carbamoyl)-4-hydroxypyrrolidin-1-yl)-3,3-dimethyl-1-oxobutan-2-yl)carbamate

To a solution of (2S,4R)-1-[(2S)-2-(tert-butoxycarbonylamino)-3,3-dimethyl-butanoyl]-4-hydroxy-pyrrolidine-2-carboxylic acid (855 mg, 2.48 mmol, CAS #630421-46-4) in DMF (2 mL) was added HATU (1.23 g, 3.23 mmol) and DIEA (1.60 g, 12.4 mmol, 2.16 mL) at 0° C. Then was added 2-[(1S)-1-aminoethyl]-5-ethynyl-phenol (400 mg, 2.48 mmol, Intermediate IG) was added and the mixture was stirred at 0° C. for 0.5 hr. On completion, the reaction mixture was filtered to give the filtrate. The filtrate was purified by prep-HPLC (0.1% FA condition) to give the title compound (570 mg, 47% yield) as a white solid. LC-MS (ESI⁺) m/z 488.3 (M+H)⁺; ¹H NMR (400 MHz, DMSO-d₆) δ=9.80 (s, 1H), 8.35-8.24 (m, 1H), 7.11 (d, J=7.6 Hz, 1H), 6.86 (dd, J=1.6, 7.6 Hz, 1H), 6.84 (d, J=1.6 Hz, 1H), 6.47-6.31 (m, 1H), 5.15-5.09 (m, 1H), 5.06 (t, J=7.6 Hz, 1H), 4.45 (t, J=8.0 Hz, 1H), 4.32-4.23 (m, 1H), 4.12 (d, J=7.2 Hz, 1H), 4.04 (s, 1H), 3.63-3.49 (m, 2H), 2.07-1.96 (m, 1H), 1.81-1.71 (m, 1H), 1.38 (s, 9H), 1.25 (d, J=7.2 Hz, 3H), 0.94-0.90 (m, 9H).

### Step 2—(2S,4R)-1-((S)-2-amino-3,3-dimethylbutanoyl)-N—((S)-1-(4-ethynyl-2-hydroxyphenyl)ethyl)-4-hydroxypyrrolidine-2-carboxamide

To a solution of tert-butyl N-[(1S)-1-[(2S,4R)-2-[[(1S)-1-(4-ethynyl-2-hydroxy-phenyl)ethyl]carbamoyl]-4-hydroxy-pyrrolidine-1-carbonyl]-2,2-dimethyl-propyl]carbamate (380 mg, 779 μmol) in DCM (12 mL) was added TFA (4.62 g, 40.5 mmol, 3 mL). The mixture was then stirred at 0° C. for 0.5 hr. On completion, NH₃·H₂O was added to the reaction mixture until the pH was 7, then concentrated under reduced pressure to give the crude residue. The residue was purified by prep-HPLC (0.1% FA condition) to give the title compound (200 mg, 59% yield, HCl) as a yellow solid. LC-MS (ESI⁺) m/z 388.1 (M+H)⁺.

### Synthesis of (R)-8-(4-((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanoic Acid (Intermediate VL)

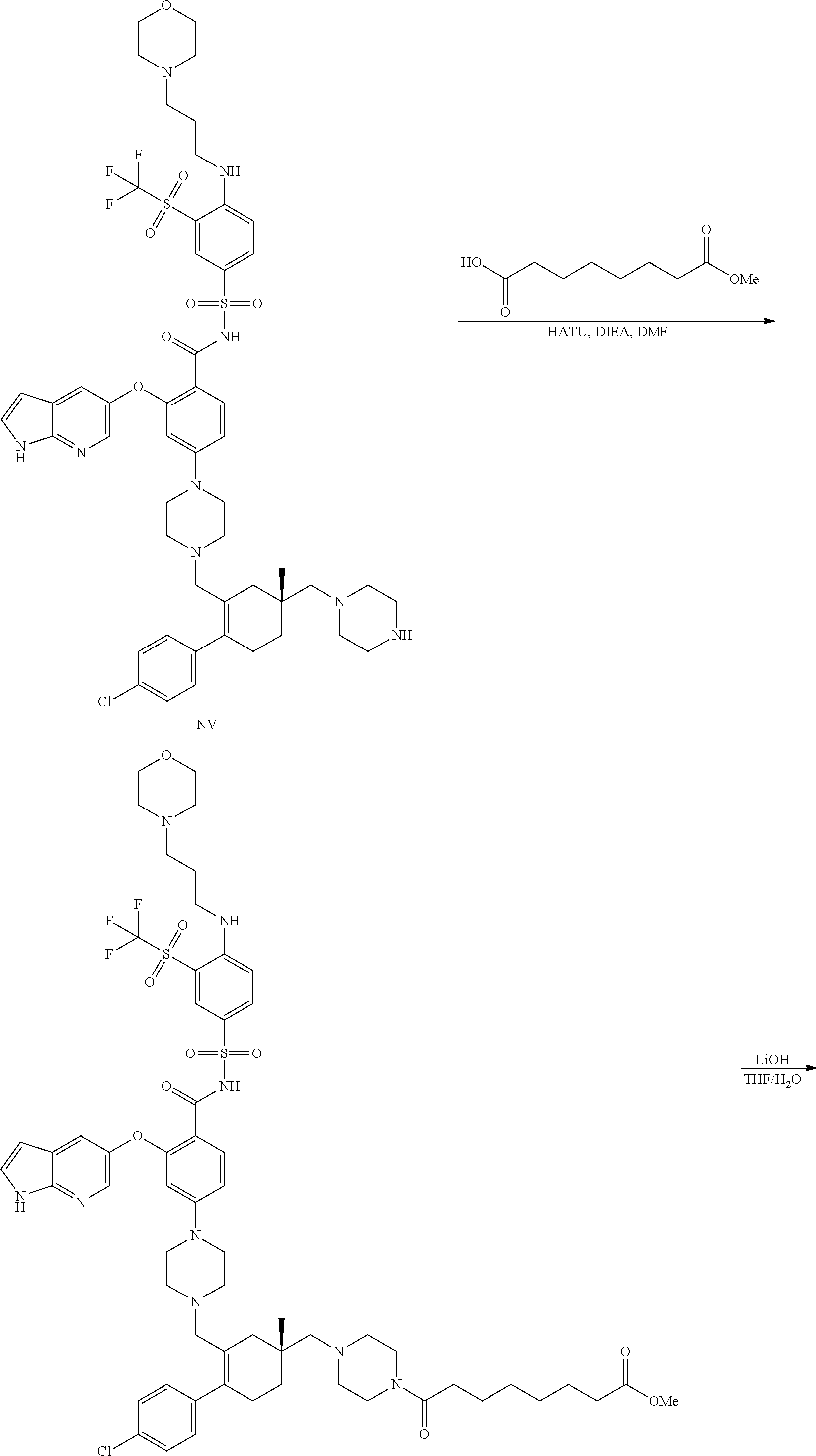

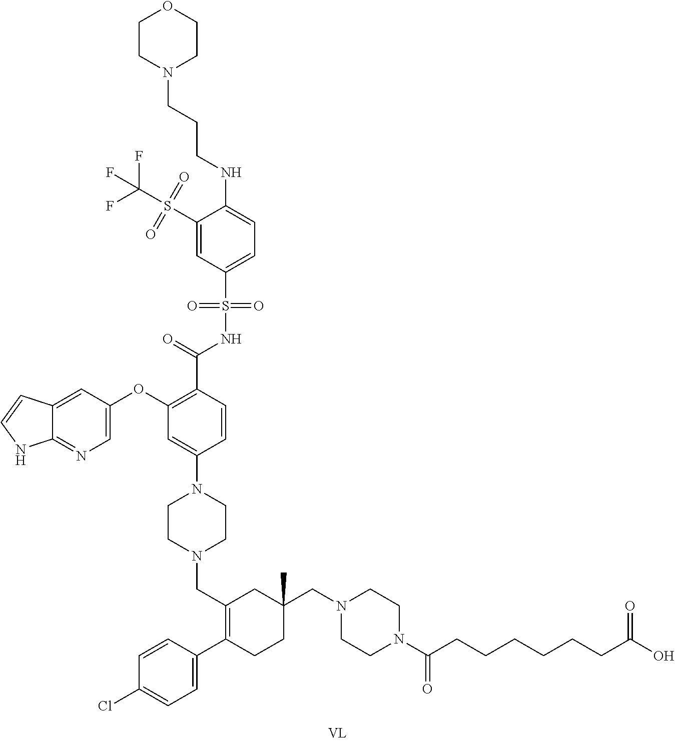 [SMILES: C[C@@]1(CN2CCN(C(=O)CCCCCCC(=O)O)CC2)CCC(c2ccc(Cl)cc2)=C(CN2CCN(c3ccc(C(=O)NS(=O)(=O)c4ccc(NCCCN5CCOCC5)c(S(=O)(=O)C(F)(F)F)c4)c(Oc4cnc5[nH]ccc5c4)c3)CC2)C1]

### Step 1—Methyl (R)-8-(4-((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanoate

To a solution of 8-methoxy-8-oxo-octanoic acid (51.1 mg, 271 μmol, CAS #3946-32-5) in DMF (2 mL) was added HATU (112 mg, 294 μmol) and DIEA (146 mg, 1.13 mmol) at 0° C., then 4-[4-[[(5R)-2-(4-chlorophenyl)-5-methyl-5-(piperazin-1-ylmethyl)cyclohexen-1-yl]methyl]piperazin-1-yl]-N-[4-(3-morpholinopropylamino)-3-(trifluoromethylsulfonyl)phenyl]sulfonyl-2-(1H-pyrrolo[2,3-b]pyridin-5-yloxy)benzamide (250 mg, 226 μmol, HCl, Intermediate NV) was added at 0° C. and the mixture was stirred at 0° C. for 0.5 hr. On completion, the reaction mixture was filtered to give the filtrate. The residue was purified by prep-HPLC (0.1% FA condition) to give the title compound (232 mg, 82% yield) as a yellow solid. LC-MS (ESI⁺) m/z 1238.6 (M+H)⁺.

### Step 2—(R)-8-(4-((6-((4-(3-((1H-pyrrolo[2,3-b]pyridin-5-yl)oxy)-4-(((4-((3-morpholinopropyl)amino)-3-((trifluoromethyl)sulfonyl)phenyl)sulfonyl)carbamoyl)phenyl)piperazin-1-yl)methyl)-4′-chloro-4-methyl-2,3,4,5-tetrahydro-[1,1′-biphenyl]-4-yl)methyl)piperazin-1-yl)-8-oxooctanoic Acid

To a solution of methyl 8-[4-[[(1R)-4-(4-chlorophenyl)-1-methyl-3-[[4-[4-[[4-(3-morpholinopropylamino)-3-(trifluoromethylsulfonyl)phenyl]sulfonylcarbamoyl]-3-(1H-pyrrolo[2,3-b]pyridin-5-yloxy)phenyl]piperazin-1-yl]methyl]cyclohex-3-en-1-yl]methyl]piperazin-1-yl]-8-oxo-octanoate (232 mg, 187 μmol) in THF (4 mL), MeOH (2 mL) and H₂O (2 mL) was added LiOH·H2O (39.3 mg, 936 μmol). The mixture was stirred at 25° C. for 2 hrs. On completion, HCl (1N) was added to the reaction mixture until the pH was 7, then the mixture was diluted with water (4 mL) and extracted with ethyl acetate (4× 10 mL). The combined organic layers were dried over anhydrous sodium sulfate, filtered and the filtrate was concentrated in vacuo to give the title compound (186 mg) as a white solid. LC-MS (ESI⁺) m/z 613.3 (M+2H)⁺/2.

### Synthesis of 8-(((S)-1-((2S,4R)-2-(((R)-1-(4-cyanophenyl)-2-hydroxyethyl)carbamoyl)-4-hydroxypyrrolidin-1-yl)-3,3-dimethyl-1-oxobutan-2-yl)amino)-8-oxooctanoic Acid (Intermediate VM)

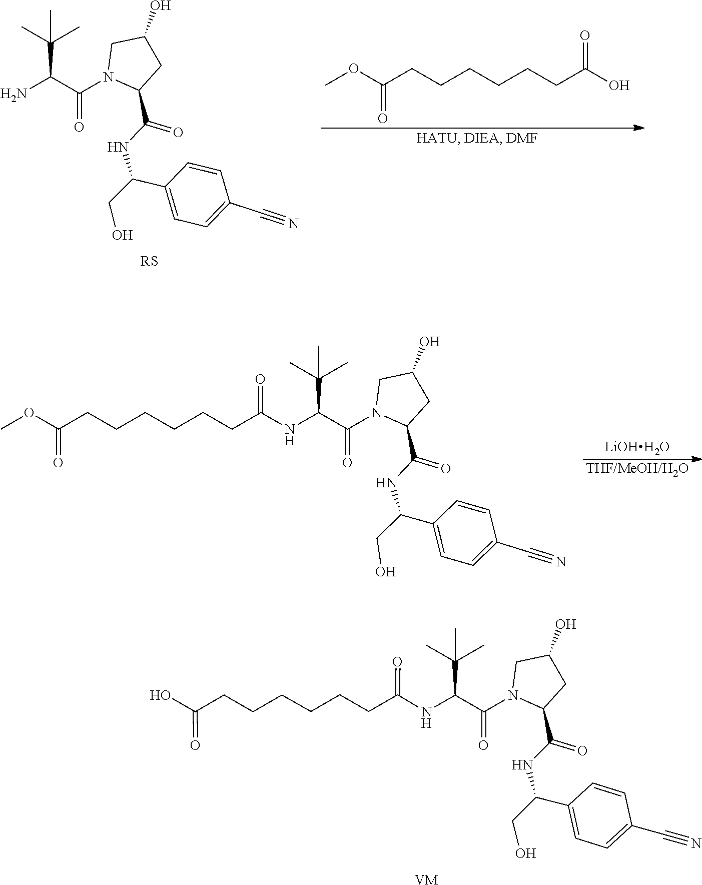

### Step 1—Methyl 8-(((S)-1-((2S,4R)-2-(((R)-1-(4-cyanophenyl)-2-hydroxyethyl)carbamoyl)-4-hydroxypyrrolidin-1-yl)-3,3-dimethyl-1-oxobutan-2-yl)amino)-8-oxooctanoate

To a solution of 8-methoxy-8-oxooctanoic acid (94.8 mg, 551 μmol, CAS ##3946-32-5) in DMF (2 mL) was added HATU (177 mg, 466 μmol) and DIEA (274 mg, 2.12 mmol). Then (2S,4R)-1-((S)-2-amino-3,3-dimethylbutanoyl)-N—((R)-1-(4-cyanophenyl)-2-hydroxyethyl)-4-hydroxypyrrolidine-2-carboxamide (180 mg, 424 μmol, HCl, Intermediate RS) was added at 0° C. and the mixture was stirred at 25° C. for 15 min. On completion, the reaction mixture was filtered to give a filtrate. The filtrate was purified by reversed-phase HPLC (0.1% FA condition) to the title compound (120 mg, 48% yield, FA) as a white solid. LC-MS (ESI⁺) m/z 559.4 (M+H)⁺.

### Step 2—8-(((S)-1-((2S,4R)-2-(((R)-1-(4-cyanophenyl)-2-hydroxyethyl)carbamoyl)-4-hydroxypyrrolidin-1-yl)-3,3-dimethyl-1-oxobutan-2-yl)amino)-8-oxooctanoic Acid

To a solution of methyl 8-(((S)-1-((2S,4R)-2-(((R)-1-(4-cyanophenyl)-2-hydroxyethyl)carbamoyl)-4-hydroxypyrrolidin-1-yl)-3,3-dimethyl-1-oxobutan-2-yl)amino)-8-oxooctanoate (120 mg, 215 μmol) in MeOH (0.5 mL), THF (2 mL) and H₂O (0.5 mL) was added LiOH·H₂O (45.1 mg, 1.07 mmol). The mixture was then stirred at 25° C. for 2 hrs. On completion, the reaction mixture was quenched with 1M HCl at 25° C. until the pH=5, and then diluted with H₂O (1 mL) and extracted with dichloromethane (15 mL). The combined organic layers were dried over anhydrous sodium sulfate, filtered and the filtrate was concentrated under reduced pressure to give the title compound (80 mg) as a white solid. LC-MS (ESI⁺) m/z 545.3 (M+H)⁺.

### Synthesis of (R)-4-(4-ethynylphenyl)-5,5-dimethyloxazolidin-2-one (Intermediate VN) and (S)-4-(4-ethynylphenyl)-5,5-dimethyloxazolidin-2-one (Intermediate VO)

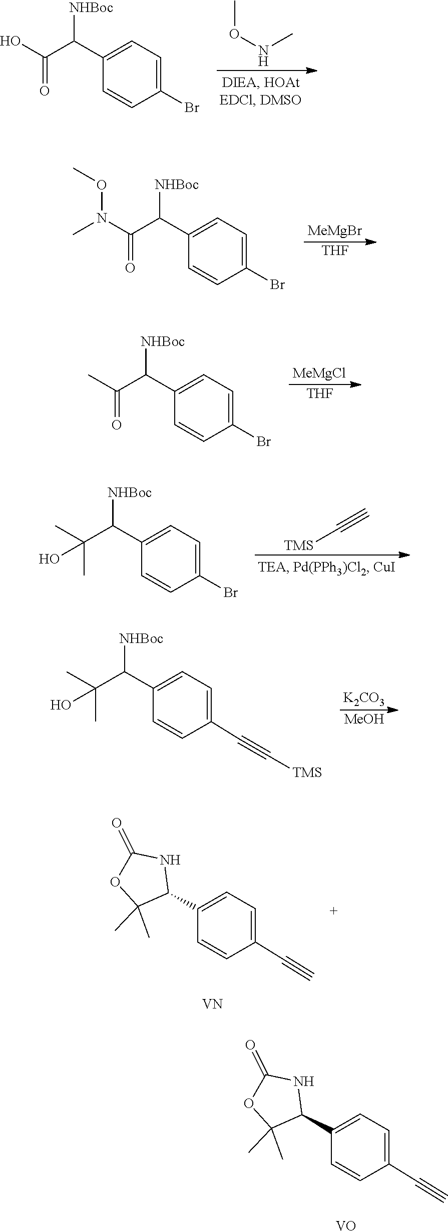

### Step 1—(R)-tert-butyl (1-(4-bromophenyl)-2-(methoxy(methyl)amino)-2-oxoethyl)carbamate

To a solution of 2-(4-bromophenyl)-2-(tert-butoxycarbonylamino)acetic acid (4.5 g, 14 mmol, CAS #1228570-47-5) in DMSO (20 mL) was added DIEA (8.81 g, 68.1 mmol, 11.8 mL), N-methoxymethanamine (1.60 g, 16.3 mmol, HCl), EDCI (3.92 g, 20.4 mmol) and HOAt (2.78 g, 20.4 mmol). The mixture was stirred at 25° C. for 12 hrs. On completion, the reaction mixture was extracted with EA (3×50 mL). The organic layer was washed with brine (100 mL), dried over Na₂SO₄, filtered and concentrated. The residue was purified by flash silica gel chromatography (ISCO®; 40 g SepaFlash® Silica Flash Column, Eluent of 0˜10% Ethyl acetate/Petroleum ether gradient (@ 60 mL/min) to give title compound (3 g, 58% yield) as a white solid. LC-MS (ESI+) m/z 319.1 (M+H)⁺.

### Step 2—Tert-butyl (1-(4-bromophenyl)-2-oxopropyl)carbamate

To a solution of MeMgBr (3 M, 24.0 mL) in THF (30 mL) was added tert-butyl N-[1-(4-bromophenyl)-2-[methoxy(methyl)amino]-2-oxo-ethyl]carbamate (2.69 g, 7.21 mmol) at 25° C. The mixture was then stirred at 25° C. for 1 hr. On completion, the reaction mixture was poured into saturated NH₄Cl aqueous solution (100 mL) and extracted with EA (100 mL×3). The combined organic layer was washed with brine (100 mL), dried over Na₂SO₄, filtered and concentrated. The residue was purified by flash silica gel chromatography (ISCO®; 40 g SepaFlash® Silica Flash Column, Eluent of 0˜10% Ethyl acetate/Petroleum ether gradient @100 mL/min) to give title compound (2.21 g, 92% yield) as a yellows solid. LC-MS (ESI+) m/z 327.0 (M+H)⁺.

### Step 3—Tert-butyl (1-(4-bromophenyl)-2-hydroxy-2-methylpropyl)carbamate

To a solution of MeMgCl (3 M, 20.4 mL) in THF (40 mL) was added tert-butyl N-[1-(4-bromophenyl)-2-oxo-propyl]carbamate (2.01 g, 6.12 mmol) at 25° C. The mixture was then stirred at 25° C. for 1 hr. The reaction mixture was poured into saturated NH₄Cl aqueous solution (100 mL) and extracted with EA (100 mL×3). The combined organic layer was washed with brine (100 mL), dried over Na₂SO₄, filtered and concentrated. The residue was purified by flash silica gel chromatography (ISCO®; 40 g SepaFlash® Silica Flash Column, Eluent of 0˜10% Ethyl acetate/Petroleum ether gradient, 60 mL/min) to give title compound (1.7 g, 80% yield) as a white solid. LC-MS (ESI+) m/z 343.1 (M+H)⁺.

### Step 4—Tert-butyl (2-hydroxy-2-methyl-1-(4-((trimethylsilyl)ethynyl)phenyl)propyl)carbamate

To a solution of tert-butyl N-[1-(4-bromophenyl)-2-hydroxy-2-methyl-propyl]carbamate (1.54 g, 4.46 mmol) in TEA (15 mL) was added CuI (169 mg, 891 μmol), ethynyl(trimethyl) silane (4.38 g, 44.5 mmol, 6.18 mL) and Pd(PPh₃)₂Cl₂ (312 mg, 445 μmol). The mixture was stirred at 80° C. for 12 hrs. On completion, the reaction mixture was extracted with EA (3×50 mL). The organic layer was washed with brine (100 mL), dried over Na₂SO₄, filtered and concentrated. The residue was purified by flash silica gel chromatography (ISCO®; 20 g SepaFlash®; Silica Flash Column, Eluent of 0˜10% Ethyl acetate/Petroleum ether gradient @100 mL/min) to give title compound (1.15 g, 57% yield) as a yellow solid. LC-MS (ESI+) m/z 288.5 (M+H)⁺.

### Step 5—(R)-4-(4-ethynylphenyl)-5,5-dimethyloxazolidin-2-one and (S)-4-(4-ethynylphenyl)-5,5-dimethyloxazolidin-2-one

To a solution of tert-butyl N-[2-hydroxy-2-methyl-1-[4-(2-trimethylsilylethynyl)phenyl]propyl]carbamate (1.15 g, 3.18 mmol) in MeOH (10 mL) was added K₂CO₃ (879 mg, 6.36 mmol). The mixture was the stirred at 25° C. for 1 hr. On completion, the reaction mixture was concentrated under vacuum. The reaction mixture was extracted with EA (3×30 mL). The organic layer was washed with brine (3×30 mL), dried over Na₂SO₄, filtered and concentrated. The crude product was purified by reversed-phase HPLC (0.1% FA condition). The product was then separated by SFC (Column: Chiralpak AD-3 50×4.6 mm I.D., 3 um Mobile phase: Phase A for CO₂, and Phase B for MeOH (0.05% DEA); Gradient elution: B in A from 5% to 40% Flow rate: 3 mL/min; Detector: PDA: column Temp: 35° C.; Back Pressure: 100 Bar) to give (R)-4-(4-ethynylphenyl)-5,5-dimethyloxazolidin-2-one (367 mg, 54% yield) as a white solid and (S)-4-(4-ethynylphenyl)-5,5-dimethyloxazolidin-2-one (223 mg, 33% yield) as a white solid. LC-MS (ESI+) m/z 216.1 (M+H)⁺. Absolute stereochemistry of the enantiomers was assigned arbitrarily.

### Synthesis of (1R)-1-amino-1-(4-ethynylphenyl)-2-methyl-propan-2-ol (Intermediate VP)

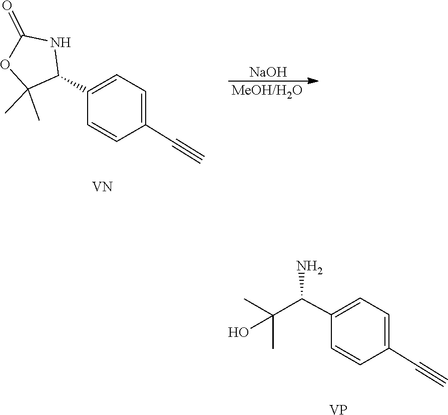 [SMILES: C#Cc1ccc([C@H]2NC(=O)OC2(C)C)cc1 ; "NaOH" ; "MeOH/H2O" ; "VN" ; C#Cc1ccc([C@@H](N)C(C)(C)O)cc1 ; "VP"]

To a solution of (4R)-4-(4-ethynylphenyl)-5,5-dimethyl-oxazolidin-2-one (0.1 g, 500 μmol, Intermediate VN) in MeOH (3 mL) and H₂O (1 mL) was added NaOH (241 mg, 6.04 mmol). The mixture was then stirred at 85° C. for 12 hrs. On completion, the reaction mixture was concentrated under vacuum. The residue was purified by prep-HPLC (column: Welch Xtimate C18 150*25 mm*5 um; mobile phase: [water (0.05% basic)-ACN]; B %: 24%-54%, 10 min) to give title compound (40 mg, 35% yield) as a white solid. LC-MS (ESI+) m/z 173.3 (M-16)⁺.

### Synthesis of (S)-1-amino-1-(4-ethynylphenyl)-2-methylpropan-2-ol (Intermediate VQ)

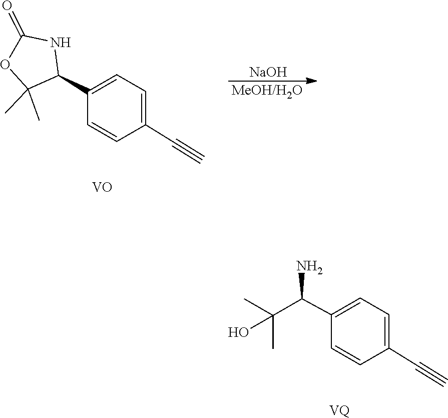

To a solution of (4S)-4-(4-ethynylphenyl)-5,5-dimethyl-oxazolidin-2-one (100 mg, 500 μmol, Intermediate VO) in MeOH (3 mL) and H₂O (1 mL) was added NaOH (241 mg, 6.04 mmol). The mixture was then stirred at 85° C. for 12 hrs. On completion, the reaction mixture was concentrated under vacuum. The residue was purified by prep-HPLC (column: Welch Xtimate C18 150*25 mm*5 um; mobile phase: [water (0.05% basic)-ACN]; B %: 24%-54%, 10 min) to give title compound (40 mg, 45% yield) as a yellow solid. LC-MS (ESI+) m/z 173.0 (M-16)⁺.

### Synthesis of (2S,4R)-1-((S)-2-amino-3,3-dimethylbutanoyl)-N—((S)-1-(4-ethynylphenyl)-2-hydroxy-2-methylpropyl)-4-hydroxypyrrolidine-2-carboxamide (Intermediate VR)

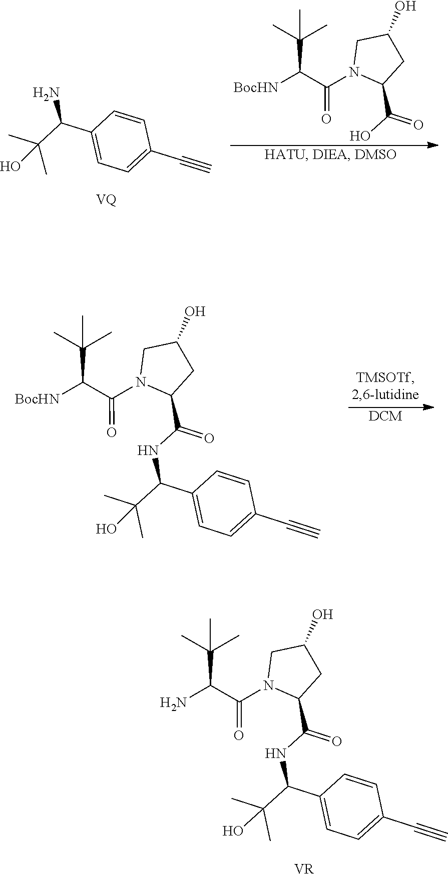

### Step 1—Tert-butyl ((S)-1-((2S,4R)-2-(((S)-1-(4-ethynylphenyl)-2-hydroxy-2-methylpropyl)carbamoyl)-4-hydroxypyrrolidin-1-yl)-3,3-dimethyl-1-oxobutan-2-yl)carbamate

To a solution of (2S,4R)-1-((S)-2-((tert-butoxycarbonyl)amino)-3,3-dimethylbutanoyl)-4-hydroxypyrrolidine-2-carboxylic acid (546 mg, 1.59 mmol, CAS #630421-46-4) in DMSO (2 mL) was added HATU (784 mg, 2.06 mmol), DIEA (1.02 g, 7.93 mmol), and (S)-1-amino-1-(4-ethynylphenyl)-2-methylpropan-2-ol (300 mg, 1.59 mmol, Intermediate VQ). The mixture was then stirred at 25° C. for 0.5 hr. On completion, the reaction mixture was filtered to give the filtrate. The crude residue was purified by reversed-phase HPLC (0.1% FA condition) to give the title compound (380 mg) as a white solid, ¹H NMR (400 MHz, DMSO-d₆) δ=8.13 (d, J=9.2 Hz, 1H), 7.36 (d, J=8.0 Hz, 4H), 6.34 (d, J=9.2 Hz, 1H), 5.12 (d, J=2.8 Hz, 1H), 4.68 (d, J=9.2 Hz, 1H), 4.62-4.57 (m, 1H), 4.56 (s, 1H), 4.31 (s, 1H), 4.12-4.07 (m. 2H), 3.63-3.58 (m, 1H), 3.56-3.51 (m, 1H), 2.05-1.95 (m, 2H), 1.37 (s, 9H), 1.05 (s, 3H), 0.98 (s, 3H), 0.84 (s, 9H). LC-MS (ESI⁺) m/z 516.2 (M+H)⁺.

### Step 2—(2S,4R)-1-((S)-2-amino-3,3-dimethylbutanoyl)-N—((S)-1-(4-ethynylphenyl)-2-hydroxy-2-methylpropyl)-4-hydroxypyrrolidine-2-carboxamide

To a solution of tert-butyl tert-butyl ((S)-1-((2S,4R)-2-(((S)-1-(4-ethynylphenyl)-2-hydroxy-2-methylpropyl)carbamoyl)-4-hydroxypyrrolidin-1-yl)-3,3-dimethyl-1-oxobutan-2-yl)carbamate (260 mg, 504 μmol) in DCM (3 mL) was added TMSOTf (336, 1.51 mmol) and 2,6-lutidine (270 mg, 2.52 mmol). The mixture was stirred at 0° C. for 0.5 hr. On completion, the reaction mixture was concentrated under reduced pressure to give a residue. The crude residue was purified by reversed-phase HPLC (0.1% FA condition) to give the title compound (150 mg) as a white solid. ¹H NMR (400 MHz, DMSO-d₆) δ=8.26 (d, J=9.2 Hz, 1H), 7.41 (s, 4H), 7.35 (d, J=1.8 Hz, 1H), 4.71-4.64 (m, 2H), 4.58 (s, 1H), 4.35 (s, 1H), 4.12 (s, 1H), 4.04 (q, J=7.2 Hz, 1H), 3.62 (d, J=16.8 Hz, 2H), 3.53 (d, J=4.0 Hz, 1H), 3.50 (d, J=4.0 Hz, 1H), 2.12-2.05 (m, 1H), 1.99 (s, 1H), 1.96-1.92 (m, 1H), 1.18 (t, J=7.2 Hz, 1H), 1.06 (s, 2H), 0.99 (s, 3H), 0.90 (s, 6H), 0.83 (s, 2H). LC-MS (ESI⁺) m/z 416.3 (M+H)⁺.

### Synthesis of 8-(((S)-1-((2S,4R)-2-(((S)-1-(4-ethynylphenyl)-2-hydroxy-2-methylpropyl)carbamoyl)-4-hydroxypyrrolidin-1-yl)-3,3-dimethyl-1-oxobutan-2-yl)amino)-8-oxooctanoic Acid (Intermediate VS)

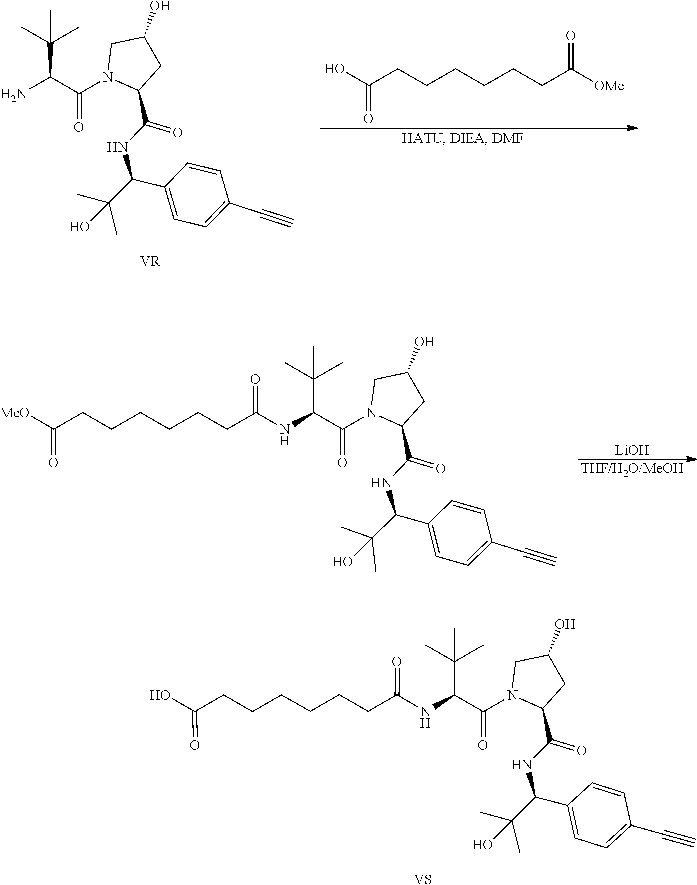

### Step 1—Methyl 8-(((S)-1-((2S,4R)-2-(((S)-1-(4-ethynylphenyl)-2-hydroxy-2-methylpropyl)carbamoyl)-4-hydroxypyrrolidin-1-yl)-3,3-dimethyl-1-oxobutan-2-yl)amino)-8-oxooctanoate

To a solution of 8-methoxy-8-oxooctanoic acid (70.7 mg, 375.43 μmol, CAS #3946-32-5) in DMF (2 mL) was added HATU (155 mg, 407 μmol), DIEA (202 mg, 1.56 mmol) and (2S,4R)-1-((S)-2-amino-3,3-dimethylbutanoyl)-N—((S)-1-(4-ethynylphenyl)-2-hydroxy-2-methylpropyl)-4-hydroxypyrrolidine-2-carboxamide (130 mg, 313 μmol, Intermediate VR). The mixture was then stirred at 0° C. for 0.5 hr. On completion, the reaction mixture was filtered to give the filtrate. The crude residue was purified by reversed-phase HPLC (0.1% FA condition) to give the title compound (70 mg) as a white solid. LC-MS (ESI⁺) m/z 586.4 (M+H)⁺.

### Step 2—8-(((S)-1-((2S,4R)-2-(((S)-1-(4-ethynylphenyl)-2-hydroxy-2-methylpropyl)carbamoyl)-4-hydroxypyrrolidin-1-yl)-3,3-dimethyl-1-oxobutan-2-yl)amino)-8-oxooctanoic Acid

To a solution of methyl methyl 8-(((S)-1-((2S,4R)-2-(((S)-1-(4-ethynylphenyl)-2-hydroxy-2-methylpropyl)carbamoyl)-4-hydroxypyrrolidin-1-yl)-3,3-dimethyl-1-oxobutan-2-yl)amino)-8-oxooctanoate (70 mg, 120 μmol) in THF (4 mL), H₂O (1 mL) and MeOH (1 mL) was added LiOH·H₂O (25.1 mg, 598 μmol). The mixture was then stirred at 25° C. for 2 hrs. On completion, the reaction mixture was added HCl (1 mol) until pH 7, then diluted by water (7 mL) and extracted by ethyl acetate (3×10 mL). The combined organic layers were dried over anhydrous sodium sulfate, filtered and the filtrate was concentrated in vacuo to give the title compound (56 mg) as a white solid. LC-MS (ESI⁺) m/z 572.5 (M+H)⁺.

### Synthesis of (2S,4R)-1-((S)-2-amino-3,3-dimethylbutanoyl)-N—((R)-1-(4-ethynylphenyl)-2-hydroxy-2-methylpropyl)-4-hydroxypyrrolidine-2-carboxamide (Intermediate VT)

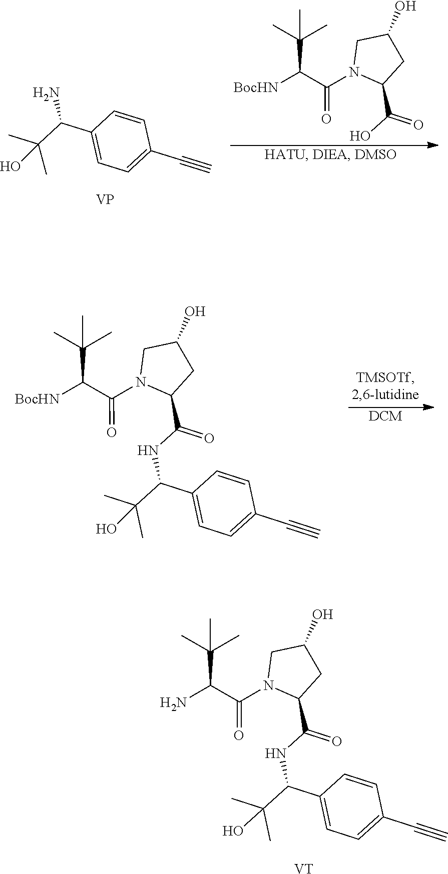

### Step 1—Tert-butyl ((S)-1-((2S,4R)-2-(((R)-1-(4-ethynylphenyl)-2-hydroxy-2-methylpropyl)carbamoyl)-4-hydroxypyrrolidin-1-yl)-3,3-dimethyl-1-oxobutan-2-yl)carbamate

To a solution of (2S,4R)-1-((S)-2-((tert-butoxycarbonyl)amino)-3,3-dimethylbutanoyl)-4-hydroxypyrrolidine-2-carboxylic acid (636 mg, 1.85 mmol, CAS #630421-46-4) in DMSO (3 mL) was added HATU (914 mg, 2.40 mmol), DIEA (1.2 g, 9.25 mmol) and (1R)-1-amino-1-(4-ethynylphenyl)-2-methyl-propan-2-ol (350 mg, 1.85 mmol, Intermediate VP). The mixture was stirred at 25° C. for 0.5 hr. On completion, the reaction mixture was filtered to give the filtrate, the solvent was removed under reduced pressure to give the crude residue. The crude residue was purified by reversed-phase HPLC (0.1% FA condition) to give the title compound (435 mg, 45% yield) as a white solid. LC-MS (ESI+) m/z 516.4 (M+H)⁺.

### Step 2—(2S,4R)-1-((S)-2-amino-3,3-dimethylbutanoyl)-N—((R)-1-(4-ethynylphenyl)-2-hydroxy-2-methylpropyl)-4-hydroxypyrrolidine-2-carboxamide

To a solution of tert-butyl ((S)-1-((2S,4R)-2-(((R)-1-(4-ethynylphenyl)-2-hydroxy-2-methylpropyl)carbamoyl)-4-hydroxypyrrolidin-1-yl)-3,3-dimethyl-1-oxobutan-2-yl)carbamate (435 mg, 843 μmol) in DCM (5 mL) was added TMSOTf (562, 2.52 mmol) and 2,6-lutidine (452 mg, 4.22 mmol). The mixture was then stirred at 0° C. for 0.5 hr. On completion, the reaction mixture was concentrated under reduced pressure to give a residue. The crude residue was purified by reversed-phase HPLC (0.1% FA condition) to give the title compound (243 mg) as a white solid. LC-MS (ESI+) m/z 416.1 (M+H)⁺.

### Synthesis of 8-(((S)-1-((2S,4R)-2-(((R)-1-(4-ethynylphenyl)-2-hydroxy-2-methylpropyl)carbamoyl)-4-hydroxypyrrolidin-1-yl)-3,3-dimethyl-1-oxobutan-2-yl)amino)-8-oxooctanoic Acid (Intermediate VU)

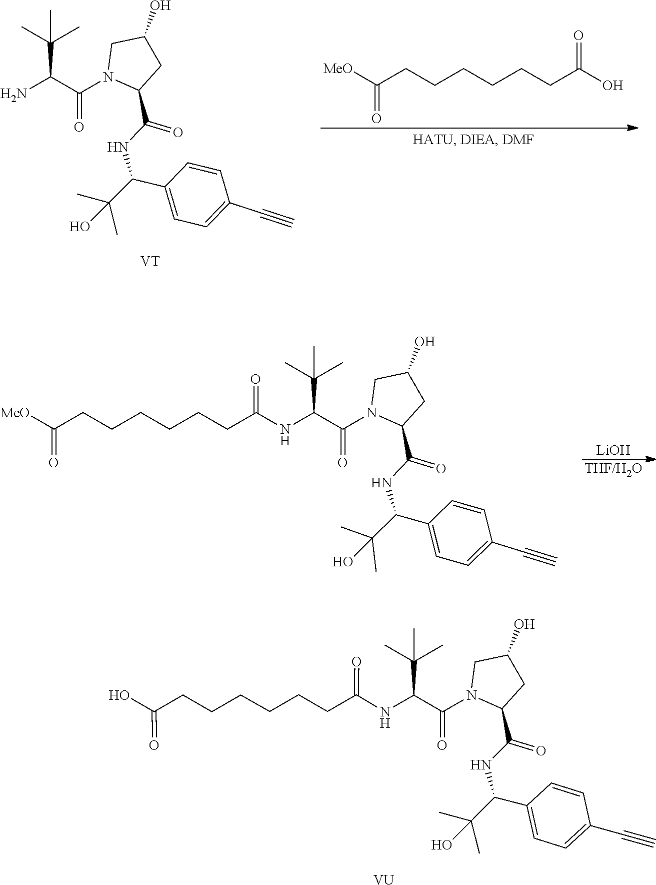

### Step 1—Methyl 8-(((S)-1-((2S,4R)-2-(((R)-1-(4-ethynylphenyl)-2-hydroxy-2-methylpropyl)carbamoyl)-4-hydroxypyrrolidin-1-yl)-3,3-dimethyl-1-oxobutan-2-yl)amino)-8-oxooctanoate

To a solution of 8-methoxy-8-oxo-octanoic acid (56.8 mg, 302 μmol) in DMF (2 mL) was added HATU (136 mg, 357 μmol) and DIEA (177 mg, 1.37 mmol) at 0° C. Then (2S,4R)-1-[(2S)-2-amino-3,3-dimethyl-butanoyl]-N-[(1R)-1-(4-ethynylphenyl)-2-hydroxy-2-methyl-propyl]-4-hydroxy-pyrrolidine-2-carboxamide (114 mg, 274 μmol, Intermediate VT) was added and the mixture was stirred at 0° C. for 0.5 hr. On completion, the reaction mixture was filtered to give the filtrate. The residue was purified by prep-HPLC (0.1% FA condition) to give the title compound (149 mg, 91% yield) as a white solid. LC-MS (ESI⁺) m/z 586.3 (M+H)⁺; ¹H NMR (400 MHz, DMSO-d₆) δ=8.30 (d, J=9.2 Hz, 1H), 7.83 (d, J=9.2 Hz, 1H), 7.40-7.32 (m, 4H), 5.07 (d, J=3.2 Hz, 1H), 4.78 (d, J=9.2 Hz, 1H), 4.57-4.49 (m, 2H), 4.41 (s, 1H), 4.27 (d, J=1.6 Hz, 1H), 4.11 (s, 1H), 3.64-3.59 (m, 2H), 3.58 (s, 3H), 2.28 (t, J=7.2 Hz, 2H), 2.25-2.19 (m, 1H), 2.10 (td, J=7.2, 14.0 Hz, 1H), 1.97-1.88 (m, 1H), 1.70 (ddd, J=4.4, 8.4, 12.8 Hz, 1H), 1.55-1.44 (m, 4H), 1.27-1.21 (m, 4H), 1.10 (s, 3H), 1.03 (s, 3H), 0.92 (s, 9H).

### Step 2—8-(((S)-1-((2S,4R)-2-(((R)-1-(4-ethynylphenyl)-2-hydroxy-2-methylpropyl)carbamoyl)-4-hydroxypyrrolidin-1-yl)-3,3-dimethyl-1-oxobutan-2-yl)amino)-8-oxooctanoic Acid

To a solution of methyl 8-[[(1S)-1-[(2S,4R)-2-[[(1R)-1-(4-ethynylphenyl)-2-hydroxy-2-methyl-propyl]carbamoyl]-4-hydroxy-pyrrolidine-1-carbonyl]-2,2-dimethyl-propyl]amino]-8-oxo-octanoate (149 mg, 254 μmol) in THF (3 mL) and H₂O (3 mL) was added LiOH·H₂O (53.4 mg, 1.27 mmol). The mixture was then stirred at 25° C. for 1 hr. On completion, the reaction mixture was added HCl (1N) until the pH was 7, then diluted with water (6 mL) and extracted with ethyl acetate (3×15 mL). The combined organic layers were dried over anhydrous sodium sulfate, filtered and the filtrate was concentrated in vacuo to give the title compound (32 mg) as a white solid. LC-MS (ESI+) m/z 572.4 (M+H)⁺.

### Synthesis of 7-(Chloromethyl)-6-(4-chlorophenyl)spiro[3.5]non-6-ene (Intermediate VV)

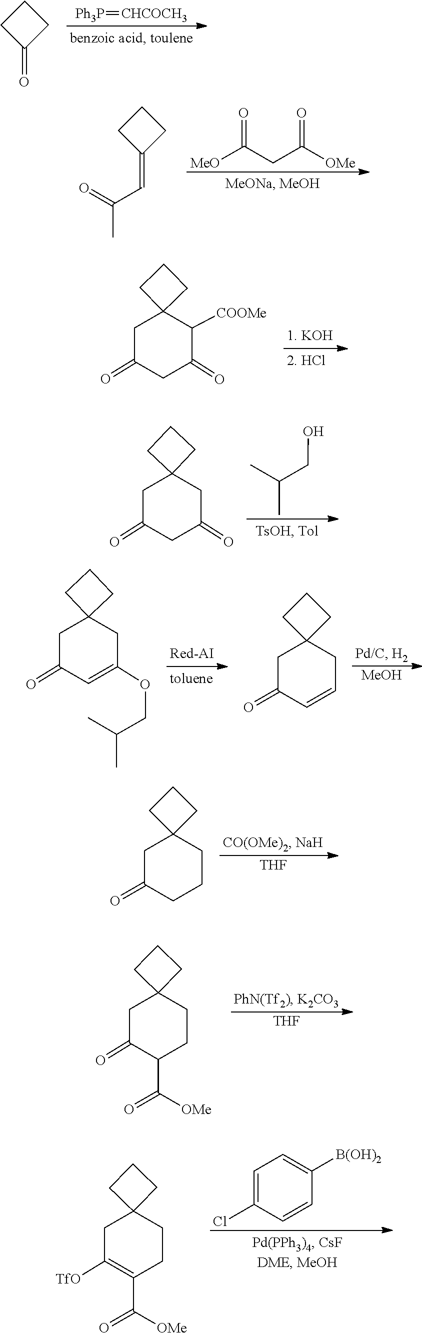

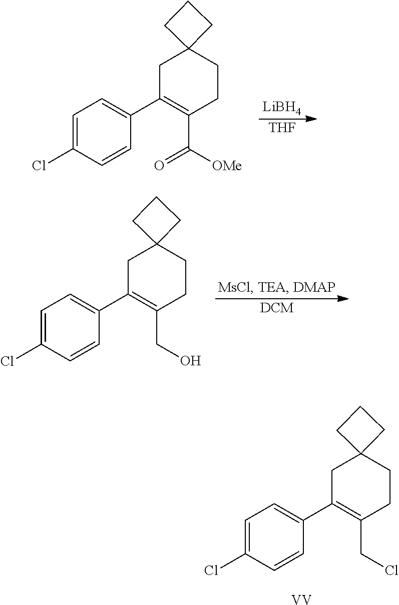

### Step 1—1-Cyclobutylidenepropan-2-one

A solution of cyclobutanone (25 g, 357 mmol, CAS #1191-95-3), 1-(triphenyl-15-phosphanylidene) propan-2-one (56.8 g, 178 mmol, CAS #1439-36-7), and benzoic acid (4.36 g, 35.7 mmol) in toluene (200 mL) was stirred at 110° C. for 12 hrs in a sealed tube. On completion, the reaction mixture was poured into the silica column. The reaction mixture was purified by column chromatography (SiO₂, Petroleum ether:Ethyl acetate=1/0 to 30/1) to give the title compound (25 g, 59% yield) as a yellow oil. ¹H NMR (400 MHz, CHLOROFORM-d) δ=5.92-5.76 (m, 1H), 3.07 (t, J=8.0 Hz, 2H), 2.85-2.75 (m, 2H), 2.08 (s, 3H), 2.07-2.01 (m, 2H).

### Step 2—Methyl 6,8-dioxospiro[3.5]nonane-5-carboxylate

To a solution of 1-cyclobutylidenepropan-2-one (20 g, 181 mmol), dimethyl malonate (26.4 g, 199 mmol, CAS #108-59-8) in MeOH (120 mL) was added NaOMe (35.9 g, 199 mmol, 30% solution). The mixture was then stirred at 70° C. for 4 hrs. On completion, the reaction mixture was concentrated in vacuo to give the title compound (40 g) as a yellow solid. LC-MS (ESI⁺) m/z 211.2 (M+H)⁺.

### Step 3—Spiro[3.5]nonane-6,8-dione

To a solution of methyl 6,8-dioxospiro[3.5]nonane-5-carboxylate (40 g, 190 mmol) in H₂O (200 mL) was added KOH (22 g, 392 mmol). The mixture was then stirred at 70° C. for 4 hrs. Then added HCl (6.94 g, 190 mmol) was added until the pH=3˜5, then the mixture was stirred at 70° C. for 4 hrs. On completion, the reaction mixture was extracted with ethyl acetate (3× 100 mL). The combined organic layers were dried over anhydrous sodium sulfate, filtered and the filtrate was concentrated in vacuo to give the residue. The residue was purified by column chromatography (SiO₂, Petroleum ether:Ethyl acetate=10/1 to 0/1) to give the title compound (25 g, 78% yield) as a yellow solid. ¹H NMR (400 MHz, DMSO-d₆) δ=11.20-10.84 (m, 1H), 5.17 (s, 1H), 2.37 (s, 4H), 1.90-1.81 (m, 2H), 1.80-1.68 (m, 4H). LC-MS (ESI⁺) m/z 153.2 (M+H)⁺.

### Step 4—8-Isobutoxyspiro[3.5]non-7-en-6-one

To a solution of spiro[3.5]nonane-6,8-dione (10 g, 65.7 mmol), 2-methylpropan-1-ol (14.6 g, 197 mmol, CAS #78-83-1) in toluene (120 mL) was added TsOH (1.13 g, 6.57 mmol). The mixture was then stirred at 130° C. for 12 hrs under N₂ atmosphere. On completion, the reaction mixture was concentrated in vacuo to give the residue. The residue was purified by column chromatography (SiO₂, Petroleum ether: Ethyl acetate=1/0 to 5/1) to give the title compound (9.4 g, 67% yield) as a yellow oil. ¹H NMR (400 MHz, CDCl₃-d) δ=5.23 (s, 1H), 3.56-3.44 (m, 2H), 2.44 (s, 2H), 2.37 (s, 2H), 2.01-1.92 (m, 1H), 1.89-1.75 (m, 6H), 0.90 (d, J=6.8 Hz, 6H). LC-MS (ESI⁺) m/z 209.2 (M+H)⁺.

### Step 5—Spiro[3.5]non-7-en-6-one

To a solution of 8-isobutoxyspiro[3.5]non-7-en-6-one (12 g, 57 mmol) in toluene (50 mL) was added Red-Al (33.3 g, 115 mmol). The mixture was then stirred at 45° C. for 24 hrs. On completion, the reaction mixture was quenched with HCl (1 M, 30 mL) at 0° C., then filtered and the filtrate was extracted with ethyl acetate (3×50 mL). The combined organic layers were washed with brine (100 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 5/1) to give the title compound (5.8 g, 66% yield) as a yellow oil. ¹H NMR (400 MHz, CHLOROFORM-d) δ=6.81 (td, J=4.0, 10.0 Hz, 1H), 5.93 (dd, J=1.6, 10.0 Hz, 1H), 2.46 (s, 2H), 2.40 (dd, J=2.0, 4.0 Hz, 2H), 1.89-1.81 (m, 2H), 1.80-1.73 (m, 4H). LC-MS (ESI⁺) m/z 137.2 (M+H)⁺.

### Step 6—Spiro[3.5]nonan-6-one

To a solution of spiro[3.5]non-7-en-6-one (5 g, 36.7 mmol) in MeOH (50 mL) was added Pd/C (1.5 g, 36.7 mmol, 10 wt %) under N₂. The suspension was degassed under vacuum and purged with H₂ several times. The mixture was then stirred under H₂ (15 psi) at 25° C. for 12 hrs. On completion, the reaction was filtered through kicsclguhr very carefully, and the filtrate was concentrated in vacuo to give the title compound (4.3 g) as a yellow oil. ¹H NMR (400 MHz, CDCl₃-d) δ=2.34 (s, 2H), 2.22-2.10 (m, 2H), 1.88-1.66 (m, 10H).

### Step 7—Methyl 6-oxospiro[3.5]nonane-7-carboxylate

To a solution of NaH (2.32 g, 57.9 mmol, 60% dispersion in mineral oil) in THF (25 mL) was added dropwise dimethyl carbonate (7.82 g, 86.8 mmol) at 25° C. After addition, spiro[3.5]nonan-6-one (4 g, 28.9 mmol) in THF (15 mL) was added dropwise at 25° C. The resulting mixture was stirred at 70° C. for 2 hrs. On completion, the reaction mixture was quenched by addition of saturated NH₄Cl aqueous solution and extracted with ethyl acetate (3×50 mL). The combined organic layers were washed with brine (50 mL) and dried over anhydrous sodium sulfate, filtered and the filtrate was concentrated in vacuo to give the residue. The residue was purified by column chromatography (SiO₂, Petroleum ether/Ethyl acetate=1/0 to 10/1) to give the title compound (4 g, 64% yield) as a yellow oil. ¹H NMR (400 MHz, CDCl₃-d) δ=3.73-3.61 (m, 3H), 2.26 (s, 2H), 2.16 (tt, J=1.2, 6.4 Hz, 2H), 1.87-1.64 (m, 7H), 1.54 (t, J=6.4 Hz, 2H). LC-MS (ESI⁺) m/z 197.1 (M+H)⁺.

### Step 8—Methyl 6-(((trifluoromethyl)sulfonyl)oxy)spiro[3.5]non-6-ene-7-carboxylate

To a solution of methyl 6-oxospiro[3.5]nonane-7-carboxylate (2 g, 10.1 mmol) in THF (30 mL) was added K₂CO₃ (4.23 g, 30.6 mmol) and 1,1,1-trifluoro-N-phenyl-N-(trifluoromethylsulfonyl)methanesulfonamide (5.46 g, 15.3 mmol). The mixture was then stirred at 60° C. for 12 hrs. On completion, the reaction mixture was diluted with water (50 mL) and extracted with ethyl acetate (3×30 mL). The combined organic lavers were dried over anhydrous sodium sulfate, filtered and the filtrate was concentrated in vacuo to give the crude residue. The residue was purified by column chromatography (SiO₂, Petroleum ether/Ethyl acetate=1/0 to 20/1) to give the title compound (3 g, 88% yield) as a colorless oil. ¹H NMR (400 MHz, CDCl₃-d) δ=3.78 (s, 3H), 2.50-2.43 (m, 4H), 1.98-1.89 (m, 2H), 1.88-1.80 (m, 4H), 1.72-1.61 (m, 2H). LC-MS (ESI⁺) m/z 329.1 (M+H)⁺.

### Step 9—Methyl 6-(4-chlorophenyl)spiro[3.5]non-6-ene-7-carboxylate

Synthesis of A mixture of methyl 6-(((trifluoromethyl)sulfonyl)oxy)spiro[3.5]non-6-ene-7-carboxylate (2.8 g, 8.53 mmol), (4-chlorophenyl)boronic acid (2.0 g, 12.8 mmol, CAS #1679-18-1), CsF (2.59 g, 17.1 mmol), and Pd(PPh₃)₄ (985 mg, 853 μmol) in DME (30 mL) and MeOH (15 mL) was degassed and purged with N₂ three times. Then the mixture was stirred at 80° C. for 4 hrs under N₂ atmosphere. On completion, the reaction mixture was concentrated in vacuo to give the crude residue. The residue was purified by column chromatography (SiO₂, Petroleum ether:Ethyl acetate=1/0 to 20/1) to give the title compound (2.1 g, 84% yield) as a colorless oil. ¹H NMR (400 MHz, CDCl₃-d) δ=7.34-7.22 (m, 2H), 7.11-7.00 (m, 2H), 3.47 (s, 3H), 2.50-2.44 (m, 2H), 2.44-2.42 (m, 2H), 1.99-1.80 (m, 6H), 1.74 (t, J=6.3 Hz, 2H).

### Step 10—(6-(4-Chlorophenyl)spiro[3.5]non-6-en-7-yl)methanol

To a solution of methyl 6-(4-chlorophenyl)spiro[3.5]non-6-ene-7-carboxylate (2 g, 6.88 mmol) in THF (20 mL) was degassed and purged with N₂ three times. Then LiBH₄ (449 mg, 20.6 mmol) at was added at 0° C. and the mixture was then stirred at 40° C. for 12 hrs under N₂ atmosphere. On completion, the reaction mixture was quenched by saturated NH₄Cl aqueous solution until the pH=7 at 0° C., and then extracted with ethyl acetate (3×20 mL). The combined organic layers were dried over anhydrous sodium sulfate, filtered and the filtrate was concentrated in vacuo to give the crude residue. The residue was purified by column chromatography (SiO₂, Petroleum ether/Ethyl acetate=1/0 to 5/1) to give the title compound (1.5 g, 82% yield) as a white solid. ¹H NMR (400 MHz, CDCl₃-d) 8=7.26-7.11 (m, 2H), 6.99 (d, J=8.4 Hz, 2H), 3.83 (s, 2H), 2.29-2.10 (m, 4H), 1.89-1.68 (m, 6H), 1.67-1.61 (m, 2H), 1.27 (s, 1H). LC-MS (ESI⁺) m/z 245.0 (M+H)⁻.

### Step 11—7-(Chloromethyl)-6-(4-chlorophenyl)spiro[3.5]non-6-ene

To a solution of (6-(4-chlorophenyl)spiro[3.5]non-6-en-7-yl) methanol (1.5 g, 5.7 mmol), TEA (1.16 g, 11.4 mmol), and DMAP (69.7 mg, 570 μmol) in DCM (20 mL) was added dropwise MsCl (1.31 g, 11.4 mmol) at 0° C. The resulting mixture was then stirred at 25° C. for 12 hrs. On completion, the reaction mixture was quenched by addition of saturated NaHCO₃ aqueous solution until the pH=7 at 0° C., and then extracted with DCM (20 mL×3). The combined organic layers were dried over Na₂SO₄, filtered and the filtrate was concentrated under reduced pressure to give the residue. The residue was purified by column chromatography (SiO₂, Petroleum ether/Ethyl acetate=1/0 to 10/1) to give the title compound (1.2 g, 74% yield) as a yellow oil. ¹H NMR (400 MHz, CDCl₃-d) δ=7.24 (d, J=8.4 Hz, 2H), 7.07 (d, J=8.4 Hz, 2H), 3.79 (s, 2H), 2.31-2.17 (m, 4H), 1.87-1.76 (m, 4H), 1.75-1.63 (m, 4H). LC-MS (ESI⁺) m/z 282.2 (M+H)⁻.

### Synthesis of 2-((1H-pyrrolo[2,3-b]pyridin-5-yl)oxy)-4-(4-((6-(4-chlorophenyl)spiro[3.5]non-6-en-7-yl)methyl)piperazin-1-yl)benzoic Acid (Intermediate VW)

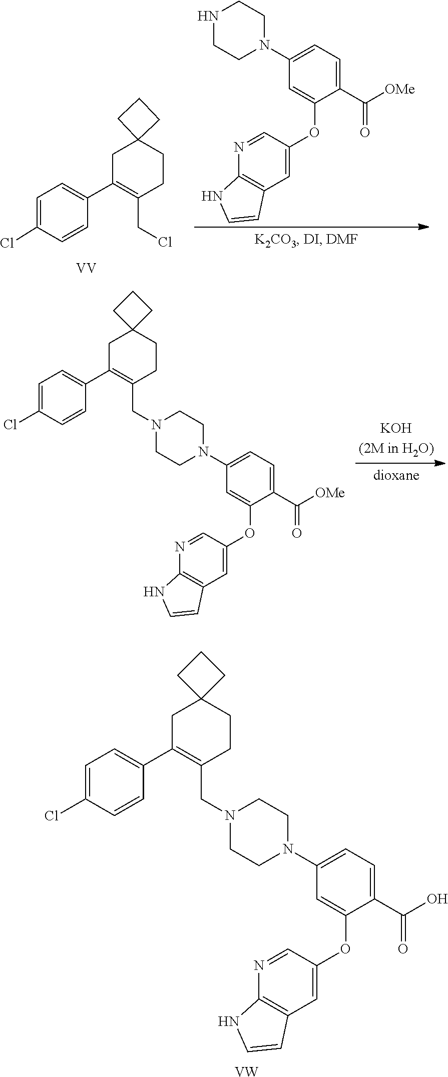

### Step 1—Methyl 2-((1H-pyrrolo[2,3-b]pyridin-5-yl)oxy)-4-(4-((6-(4-chlorophenyl)spiro[3.5]non-6-en-7-yl)methyl)piperazin-1-yl)benzoate

To a solution of 7-(chloromethyl)-6-(4-chlorophenyl)spiro[3.5]non-6-ene (1.1 g, 3.91 mmol, Intermediate VV), methyl 2-((1H-pyrrolo[2,3-b]pyridin-5-yl)oxy)-4-(piperazin-1-yl)benzoate (1.65 g, 4.69 mmol, synthesized via Step 1 of Intermediate NQ) in DMF (20 mL) was added K₂CO₃ (1.62 g, 11.7 mmol) and KI (129 mg, 782 μmol). The mixture was then stirred at 25° C. for 12 hrs. On completion, the reaction mixture was diluted with water (50 mL) and extracted with ethyl acetate (3×30 mL). The combined organic layers were washed with brine (2×30 mL) and dried over anhydrous sodium sulfate, filtered and the filtrate was concentrated in vacuo to give the crude residue. The residue was purified by column chromatography (SiO₂, Petroleum ether/Ethyl acetate=1/0 to 1/1) to give the title compound (2 g, 85% yield) as a yellow oil. LC-MS (ESI⁺) m/z 597.2 (M+H)+.

### Step 2—2-((1H-pyrrolo[2,3-b]pyridin-5-yl)oxy)-4-(4-((6-(4-chlorophenyl)spiro[3.5]non-6-en-7-yl)methyl)piperazin-1-yl)benzoic Acid

To a solution of methyl 2-((1H-pyrrolo[2,3-b]pyridin-5-yl)oxy)-4-(4-((6-(4-chlorophenyl)spiro[3.5]non-6-en-7-yl)methyl)piperazin-1-yl)benzoate (500 mg, 837 μmol) in dioxane (6 mL) was added KOH (2 M, 4.19 mL). The mixture was then stirred at 60° C. for 12 hrs. On completion, the reaction mixture was quenched by addition of HCl (1 M) until the pH-7, and then extracted with EA (20 mL×3). The combined organic layers were dried over Na₂SO₄, filtered and the filtrate was concentrated under reduced pressure to give the title compound (450 mg) as a yellow solid. ¹H NMR (400 MHz, CHLOROFORM-d) δ=10.20 (s, 1H), 8.09 (d, J=2.4 Hz, 1H), 7.92 (d, J=9.2 Hz, 1H), 7.51 (d, J=2.4 Hz, 1H), 7.25 (s, 1H), 7.19 (d, J=2.0 Hz, 2H), 6.87 (d, J=8.4 Hz, 2H), 6.54 (dd, J=2.0, 9.2 Hz, 1H), 6.33 (d, J=2.4 Hz, 1H), 6.14 (d, J=2.0 Hz, 1H), 3.63 (s, 3H), 3.05 (d, J=4.4 Hz, 4H), 2.69 (s, 2H), 2.19 (s, 6H), 1.84-1.64 (m, 6H), 1.58 (t, J=6.0 Hz, 2H). LC-MS (ESI⁺) m/z 583.1 (M+H)⁺.

### Synthesis of 2-((1H-pyrrolo[2,3-b]pyridin-5-yl)oxy)-4-(4-((6-(4-chlorophenyl)spiro[3.5]non-6-en-7-yl)methyl)piperazin-1-yl)-N-((4-((3-(piperazin-1-yl)propyl)amino)-3-((trifluoromethyl)sulfonyl)phenyl)sulfonyl)benzamide (Intermediate VX)

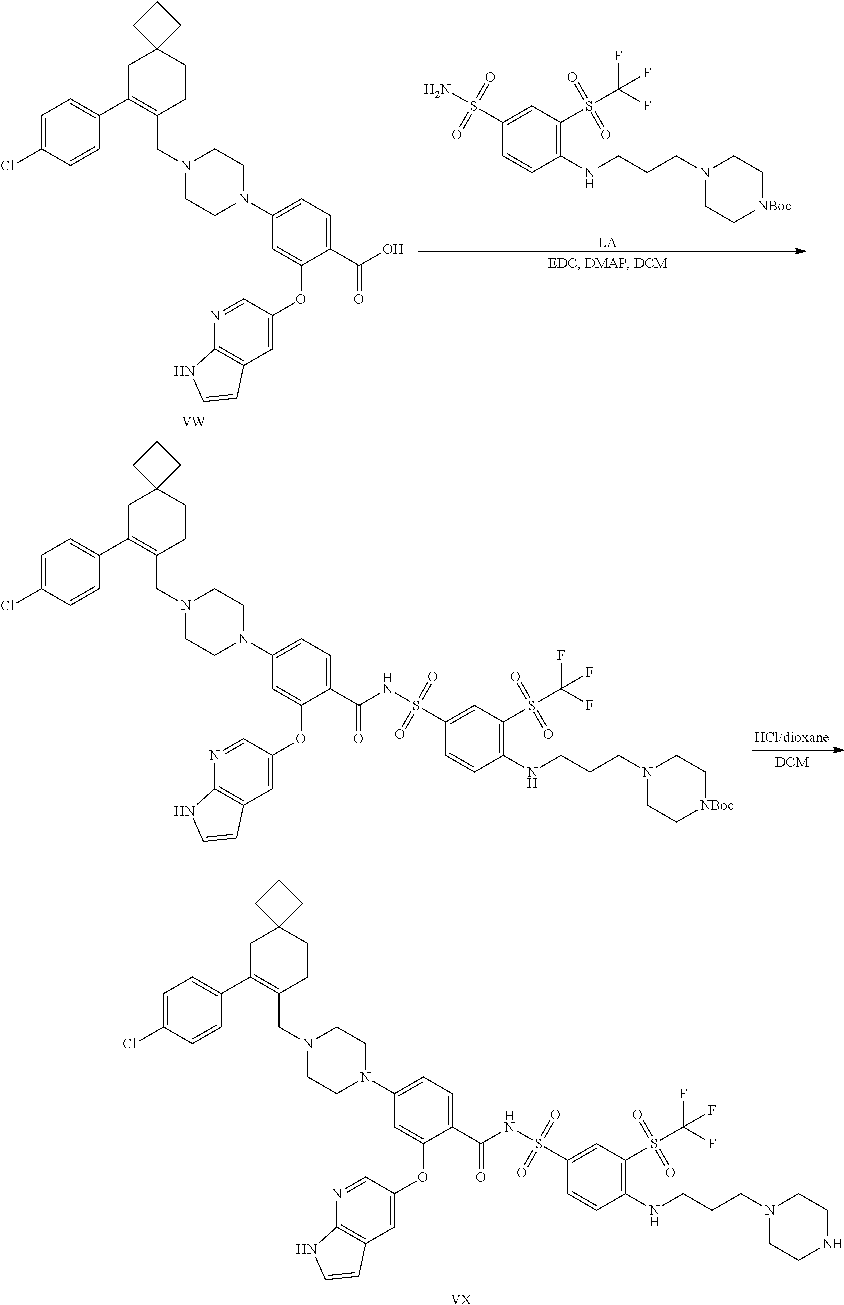

### Step 1—Tert-butyl 4-(3-((4-(N-(2-((1H-pyrrolo[2,3-b]pyridin-5-yl)oxy)-4-(4-((6-(4-chlorophenyl)spiro[3.5]non-6-en-7-yl)methyl)piperazin-1-yl)benzoyl)sulfamoyl)-2-((trifluoromethyl)sulfonyl)phenyl)amino)propyl)piperazine-1-carboxylate

To a solution of 2-((1H-pyrrolo[2,3-b]pyridin-5-yl)oxy)-4-(4-((6-(4-chlorophenyl)spiro[3.5]non-6-en-7-yl)methyl)piperazin-1-yl)benzoic acid (350 mg, 600 μmol, Intermediate VW) in DCM (6 mL) was added EDC (186 mg, 1.20 mmol) and DMAP (183 mg, 1.50 mmol) at 25° C. After addition, the mixture was stirred at this temperature for 10 min, and then tert-butyl 4-(3-((4-sulfamoyl-2-((trifluoromethyl)sulfonyl)phenyl)amino)propyl)piperazine-1-carboxylate (318 mg, 600 μmol, Intermediate LA) was added. The resulting mixture was stirred at 25° C. for 2 hrs. On completion, the reaction mixture was quenched with water (20 mL) and extracted with dichloromethane (3×10 mL). The combined organic layers were dried over anhydrous sodium sulfate, filtered and the filtrate was concentrated in vacuo to give the title compound (400 mg) as a yellow oil. (LC-MS (ESI⁺) m/z 1095.5 (M+H)⁺.

### Step 2—2-((1H-pyrrolo[2,3-b]pyridin-5-yl)oxy)-4-(4-((6-(4-chlorophenyl)spiro[3.5]non-6-en-7-yl)methyl)piperazin-1-yl)-N-((4-((3-(piperazin-1-yl)propyl)amino)-3-((trifluoromethyl)sulfonyl)phenyl)sulfonyl)benzamide

To a solution of tert-butyl 4-(3-((4-(N-(2-((1H-pyrrolo[2,3-b]pyridin-5-yl)oxy)-4-(4-((6-(4-chlorophenyl)spiro[3.5]non-6-en-7-yl)methyl)piperazin-1-yl)benzoyl)sulfamoyl)-2-((trifluoromethyl)sulfonyl)phenyl)amino)propyl)piperazine-1-carboxylate (400 mg, 365 μmol) in DCM (10 mL) was added HCl/dioxane (4 M, 2 mL). The mixture was then stirred at 25° C. for 2 hrs. On completion, the reaction mixture was concentrated in vacuo to give the crude residue. The crude residue was purified by reversed-phase HPLC (0.8 g/L ammonium bicarbonate) to give the title compound (150 mg, 34% yield) as a white solid. (LC-MS (ESI⁺) m/z 498.8 (M/2+H)⁺.

### Synthesis of (R)-2-((tert-butyldimethylsilyl)oxy)-1-(4-(prop-1-yn-1-yl)phenyl)ethan-1-amine (Intermediate VY)

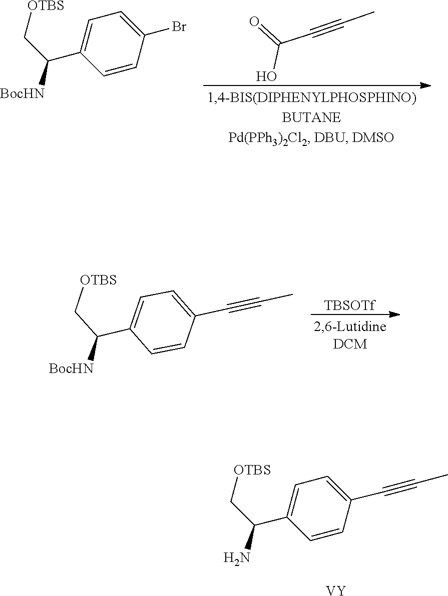

### Step 1—Tert-butyl (R)-(2-((tert-butyldimethylsilyl)oxy)-1-(4-(prop-1-yn-1-yl)phenyl)ethyl)carbamate

A mixture of tert-butyl (R)-(1-(4-bromophenyl)-2-((tert-butyldimethylsilyl)oxy)ethyl) carbamate (2 g, 4.65 mmol, synthesized via Step 1 of Intermediate RP), but-2-ynoic acid (1.17 g, 13.9 mmol, CAS #590-93-2), dichloropalladium triphenylphosphane (652 mg, 929 μmol), DBU (707 mg, 4.65 mmol, 700 uL) and 4-diphenylphosphanylbutyl(diphenyl)phosphane (198 mg, 464 μmol) in DMSO (40 mL) was degassed and purged with N₂ three times. Then the mixture was stirred at 110° C. for 12 hrs under N₂ atmosphere. On completion, the reaction mixture was quenched with water (50 mL) and extracted with dichloromethane (2×40 mL). The combined organic layers were washed with brine (40 mL), dried over anhydrous sodium sulfate, filtered and concentrated in vacuo to give the crude residue. The residue was purified by column chromatography (SiO₂, Petroleum ether:Ethyl acetate=1/0 to 10/1) to give the title compound (1.2 g, 66% yield) as a yellow oil. ¹H NMR (400 MHz, DMSO-d₆) δ=7.33-7.30 (m, 2H), 7.28-7.25 (m, 2H), 4.62-4.50 (m, 1H), 3.65-3.57 (m, 2H), 2.02 (s, 3H), 1.36 (s, 9H), 0.80 (s, 9H), −0.05 (s, 3H), −0.08 (s, 3H).

### Step 2—(R)-2-((tert-butyldimethylsilyl)oxy)-1-(4-(prop-1-yn-1-yl)phenyl)ethan-1-amine

To a solution of tert-butyl (R)-(2-((tert-butyldimethylsilyl)oxy)-1-(4-(prop-1-yn-1-yl)phenyl)ethyl)carbamate (500 mg, 1.28 mmol) in DCM (5 mL) was added TBSOTf (1.02 g, 3.85 mmol, 884 uL) and 2,6-lutidine (687 mg, 6.42 mmol, 747 uL). The mixture was then stirred at 25° C. for 12 hrs. On completion, the reaction mixture was concentrated in vacuo to give the crude residue. The residue was purified by reversed-phase HPLC (0.8 g/L ammonium bicarbonate) to give the title compound (150 mg, 40% yield) as a white solid. LC-MS (ESI⁺) m/z 290.1 (M+H)⁺.

### Synthesis of (2S,4R)-1-((S)-2-amino-3,3-dimethylbutanoyl)-4-hydroxy-N—((R)-2-hydroxy-1-(4-(prop-1-yn-1-yl)phenyl)ethyl)pyrrolidine-2-carboxamide (Intermediate VZ)

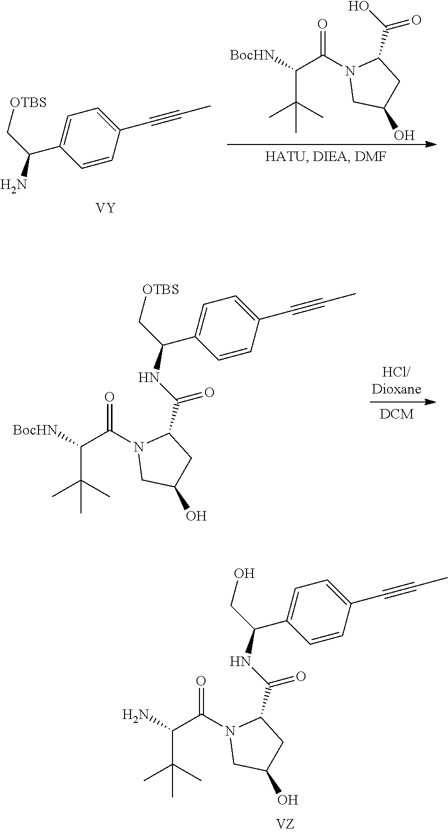

### Step 1—Tert-butyl ((S)-1-((2S,4R)-2-(((R)-2-((tert-butyldimethylsilyl)oxy)-1-(4-(prop-1-yn-1-yl)phenyl)ethyl)carbamoyl)-4-hydroxypyrrolidin-1-yl)-3,3-dimethyl-1-oxobutan-2-yl)carbamate

To a solution of ((2S,4R)-1-((S)-2-((tert-butoxycarbonyl)amino)-3,3-dimethylbutanoyl)-4-hydroxypyrrolidine-2-carboxylic acid (178 mg, 518 μmol, CAS #630421-46-4) in DMF (2 mL) was added HATU (256 mg, 673 μmol) and stirred for 5 min. Then DIEA (334 mg, 2.59 mmol, 451 uL) and (R)-2-((tert-butyldimethylsilyl)oxy)-1-(4-(prop-1-yn-1-yl)phenyl)ethan-1-amine (150 mg, 518 μmol, Intermediate VY) was added and the mixture was stirred at 25° C. for 10 min. On completion, the reaction mixture was concentrated in vacuo to give the crude residue. The residue was purified by reversed-phase HPLC (0.8 g/L ammonium bicarbonate) to give the title compound (250 mg, 78% yield) as a yellow oil. LC-MS (ESI⁺) m/z 616.4 (M+H)⁺.

### Step 2—(2S,4R)-1-((S)-2-amino-3,3-dimethylbutanoyl)-4-hydroxy-N—((R)-2-hydroxy-1-(4-(prop-1-yn-1-yl)phenyl)ethyl)pyrrolidine-2-carboxamide

To a solution of tert-butyl ((S)-1-((2S,4R)-2-(((R)-2-((tert-butyldimethylsilyl)oxy)-1-(4-(prop-1-yn-1-yl)phenyl)ethyl) carbamoyl)-4-hydroxypyrrolidin-1-yl)-3,3-dimethyl-1-oxobutan-2-yl)carbamate (250 mg, 405 μmol) in DCM (10 mL) was added HCl/dioxane (4 M, 1 mL). The mixture was then stirred at 25° C. for 0.5 hrs. On completion, the reaction mixture was concentrated in vacuo to give the title compound (250 mg) as white solid. LC-MS (ESI⁺) m/z 402.2 (M+H)⁺.

### Synthesis of Tert-butyl (R)-7-(4-(phenylthio)-3-((4-sulfamoyl-2-((trifluoromethyl)sulfonyl)phenyl)amino)butyl)-2,7-diazaspiro[3.5]nonane-2-carboxylate (Intermediate WA)

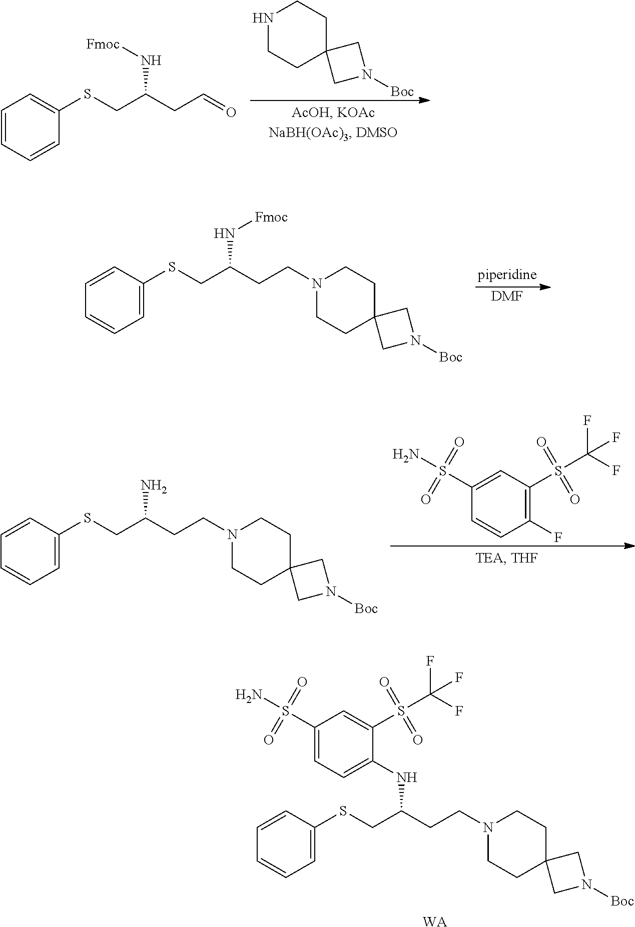

### Step 1—Tert-butyl (R)-7-(3-((((9H-fluoren-9-yl)methoxy) carbonyl)amino)-4-(phenylthio)butyl)-2,7-diazaspiro[3.5]nonane-2-carboxylate

To a solution of 9H-fluoren-9-ylmethyl N-[(1R)-3-oxo-1-(phenylsulfanylmethyl)propyl]carbamate (2.00 g, 4.79 mmol, synthesized via Steps 1-3 of Intermediate KK) in THF (30 mL) was added tert-butyl 2,7-diazaspiro[3.5]nonane-2-carboxylate (1.08 g, 4.79 mmol) (CAS #236406-55-6) and NaBH(OAc)₃ (1.02 g, 4.79 mmol). The mixture was then stirred at 25° C. for 2 hrs. On completion, the crude residue was purified by column chromatography (SiO₂, Petroleum ether: Ethyl acetate=10/1 to 0/1) to give the title compound (2.30 g, 73% yield) as a yellow oil. LC-MS (ESI⁺) m/z 628.6 (M+H)⁺.

### Step 2—Tert-butyl (R)-7-(3-amino-4-(phenylthio)butyl)-2,7-diazaspiro[3.5]nonane-2-carboxylate

To a solution of tert-butyl (R)-7-(3-((((9H-fluoren-9-yl)methoxy) carbonyl)amino)-4-(phenylthio)butyl)-2,7-diazaspiro[3.5]nonane-2-carboxylate (2.30 g, 3.66 mmol) in DMF (10 mL) was added piperidine (862 mg, 10.13 mmol, 1 mL). The mixture was then stirred at 25° C. for 12 hrs. On completion, the crude residue was purified by purified by reversed-phase HPLC (0.8 g/L ammonium bicarbonate) to give the title compound (1.00 g, 62% yield) as a yellow oil. LC-MS (ESI⁺) m/z 406.4 (M+H)⁺.

### Step 3—Tert-butyl (R)-7-(4-(phenylthio)-3-((4-sulfamoyl-2-((trifluoromethyl)sulfonyl)phenyl)amino)butyl)-2,7-diazaspiro[3.5]nonane-2-carboxylate

To a solution of tert-butyl (R)-7-(3-amino-4-(phenylthio)butyl)-2,7-diazaspiro[3.5]nonane-2-carboxylate (1.00 g, 2.47 mmol) in ACN (10 mL) was added DIEA (1.59 g, 12.3 mmol, 2.15 mL) and 4-fluoro-3-((trifluoromethyl)sulfonyl)benzenesulfonamide (1.14 g, 3.70 mmol) (CAS #1027345-08-9). The mixture was then stirred at 80° C. for 8 hrs. On completion, the crude residue was purified by column chromatography (SiO₂, Petroleum ether:Ethyl acetate=10/1 to 0/1) to give the title compound (1.00 g, 49% yield) as a white solid. LC-MS (ESI⁺) m/z 693.5 (M+H)⁺.

### Synthesis of 4-(4-((R)-(4′-chloro-[1,1′-biphenyl]-2-yl)(hydroxy)methyl)piperidin-1-yl)-N-((4-(((R)-1-(phenylthio)-4-(2,7-diazaspiro[3.5]nonan-7-yl)butan-2-yl)amino)-3-(trifluoromethyl)sulfonyl)phenyl)sulfonyl)benzamide (Intermediate WB)

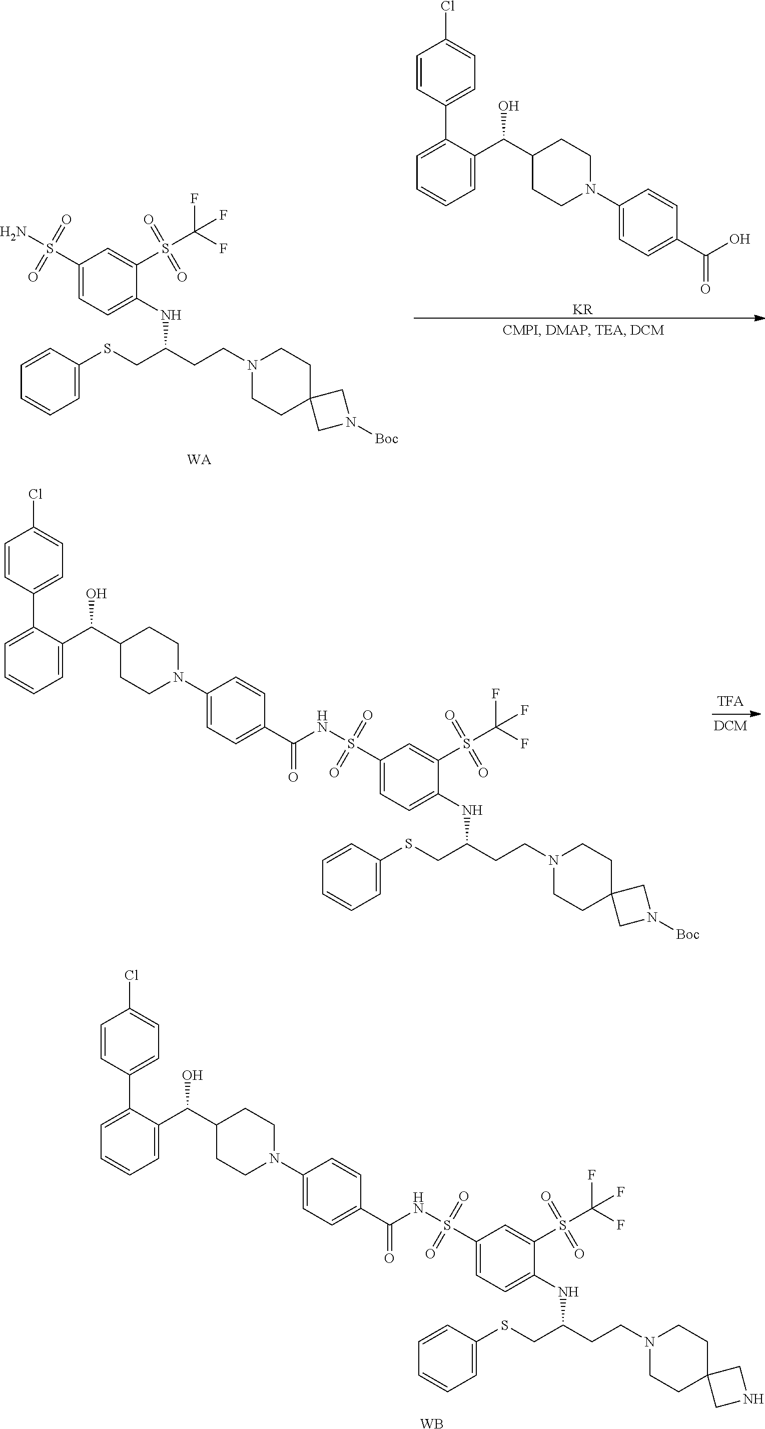

### Step 1—Tert-butyl 7-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)-2,7-diazaspiro[3.5]nonane-2-carboxylate

To a solution of (R)-4-(4-((4′-chloro-[1,1′-biphenyl]-2-yl)(hydroxy)methyl)piperidin-1-yl)benzoic acid (200 mg, 474 μmol, Intermediate KR) in DCM (6 mL) was added CMPI (182 mg, 711 μmol), DMAP (57.9 mg, 474 μmol), TEA (144 mg, 1.42 mmol, 198 uL) and tert-butyl (R)-7-(4-(phenylthio)-3-((4-sulfamoyl-2-((trifluoromethyl)sulfonyl)phenyl)amino)butyl)-2,7-diazaspiro[3.5]nonane-2-carboxylate (328 mg, 474 μmol, Intermediate WA). The mixture was then stirred at 25° C. for 2 hrs. On completion, the crude residue was purified by reversed-phase HPLC (0.1% FA condition) to give the title compound (350 mg, 64% yield) as a white solid. LC-MS (ESI⁺) m/z 1096.2 (M+H)⁺.

### Step 2—4-(4-((R)-(4′-chloro-[1,1′-biphenyl]-2-yl)(hydroxy)methyl)piperidin-1-yl)-N-((4-(((R)-1-(phenylthio)-4-(2,7-diazaspiro[3.5]nonan-7-yl)butan-2-yl)amino)-3-((trifluoromethyl)sulfonyl)phenyl)sulfonyl)benzamide

A solution of tert-butyl 7-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)-2,7-diazaspiro[3.5]nonane-2-carboxylate (200 mg, 182 μmol), TMSOTf (405 mg, 1.82 mmol), and 2,6-lutidine (195 mg, 1.82 mmol, 212 uL) in DCM (0.2 mL) and the mixture was stirred at 25° C. for 2 hrs. On completion, the crude residue was purified by reversed-phase HPLC (0.1% ammonium hydroxide or 0.1% FA condition) to give the title compound (140 mg, 76% yield) as a white solid. LC-MS (ESI⁺) m/z 996.4 (M+H)⁺.

### Synthesis of (2S,4R)-1-((S)-3,3-dimethyl-2-(6-oxo-6-(2,7-diazaspiro[3.5]nonan-7-yl) hexanamido)butanoyl)-4-hydroxy-N—(S)-1-(4-(4-methylthiazol-5-yl)phenyl)ethyl)pyrrolidine-2-carboxamide (Intermediate WC)

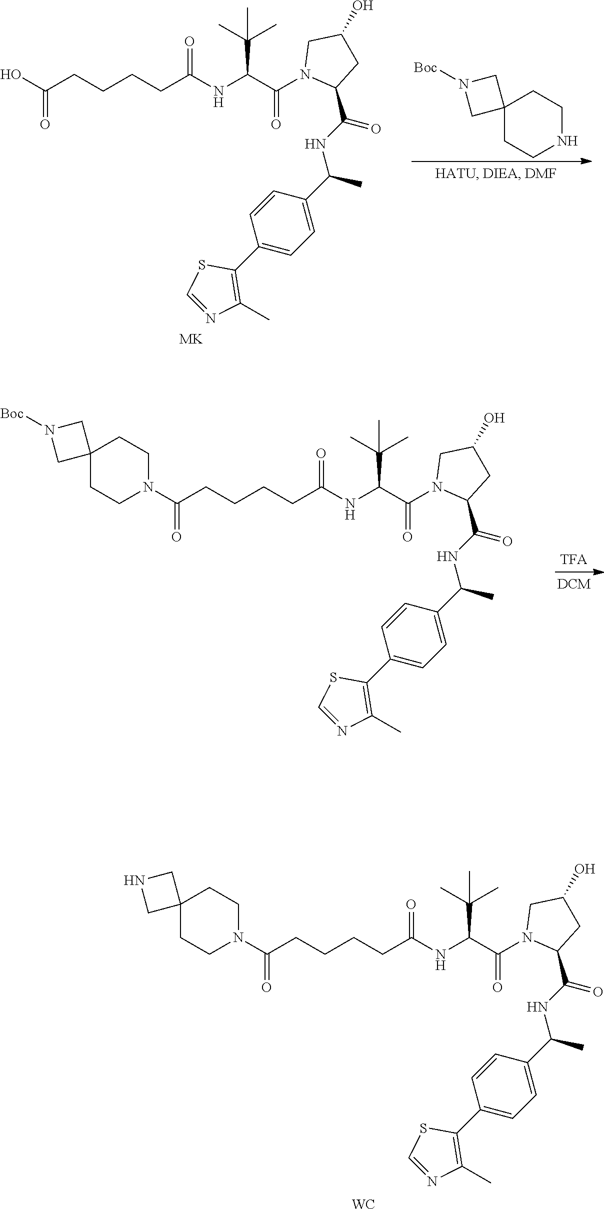

### Step 1—(2S,4R)-1-((S)-2-(6-(2-((11-boraneyl)carbonyl)-2,7-diazaspiro[3.5]nonan-7-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide

To a solution of 6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoic acid (400 mg, 698 μmol, Intermediate MK) in DMF (6 mL) was added HATU (319 mg, 838 μmol), DIEA (451 mg, 3.49 mmol, 608 uL) and (11-boraneyl) (2,712-diazaspiro[3.5]nonan-2-yl) methanone (189 mg, 838 μmol, CAS #236406-55-6). The mixture was then stirred at 25° C. for 0.5 hrs. On completion, the residue was purified by prep-HPLC (0.8 g/L ammonium bicarbonate) to give the title compound (380 mg, 94% yield) as a white solid. LC-MS (ESI⁺) m/z 781.5 (M+H)⁺.

### Step 2—(2S,4R)-1-((S)-3,3-dimethyl-2-(6-oxo-6-(2,7-diazaspiro[3.5]nonan-7-yl) hexanamido)butanoyl)-4-hydroxy-N—((S)-1-(4-(4-methylthiazol-5-yl)phenyl)ethyl)pyrrolidine-2-carboxamide

To a solution of (2S,4R)-1-((S)-2-(6-(2-((11-boraneyl) carbonyl)-2,7-diazaspiro[3.5]nonan-7-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (450 mg, 576 μmol) in DCM (4 mL) was added TFA (723 mg, 6.34 mmol, 469 uL). The mixture was then stirred at 25° C. for 12 hrs. On completion, the residue was purified by prep-HPLC (FA condition) to give the title compound (450 mg, 70% yield) as a white solid. LC-MS (ESI⁺) m/z 681.2 (M+H)⁺.

### Synthesis of (2S,4R)-1-(S)-3,3-dimethyl-2-(6-oxo-6-(2-((R)-4-(phenylthio)-3-(4-sulfamoyl-2-(trifluoromethyl)sulfonyl)phenyl)amino)butyl)-2,7-diazaspiro[3.5]nonan-7-yl)hexanamido)butanoyl)-4-hydroxy-N—((S)-1-(4-(4-methylthiazol-5-yl)phenyl)ethyl)pyrrolidine-2-carboxamide (Intermediate WD)

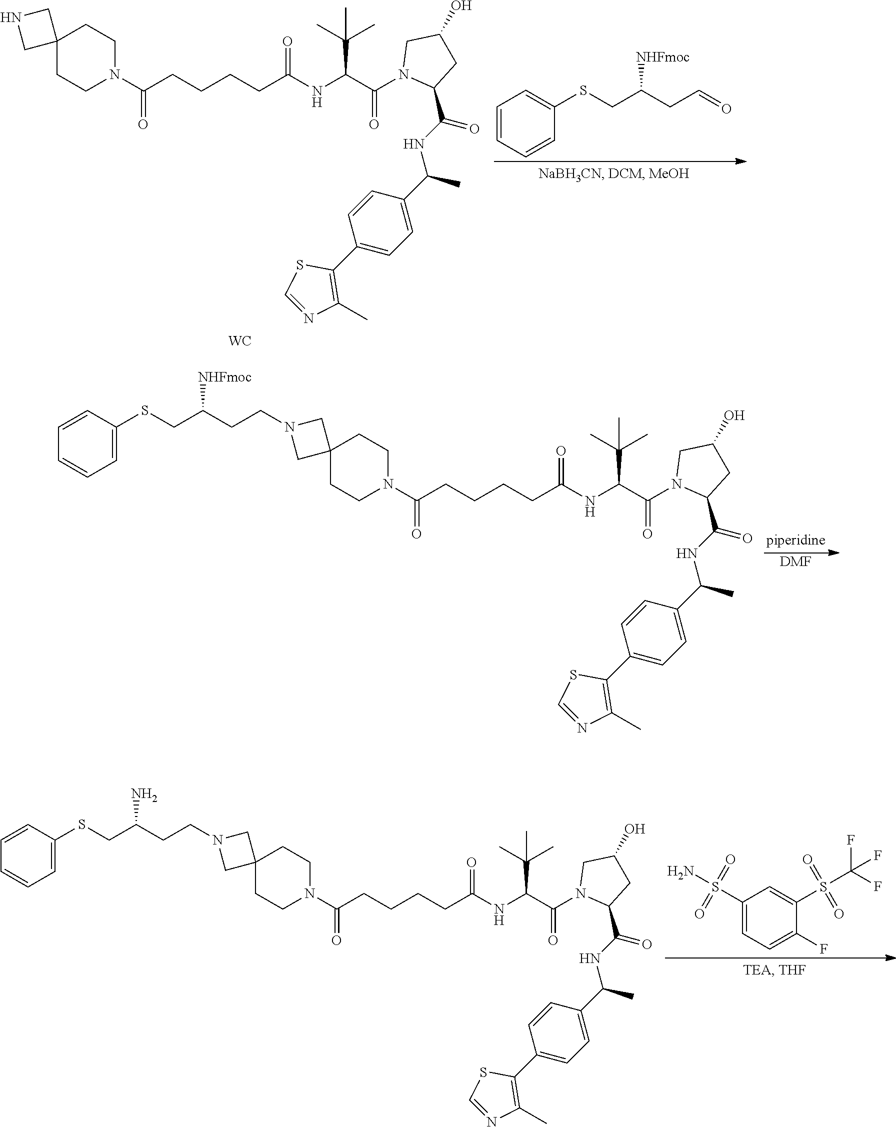

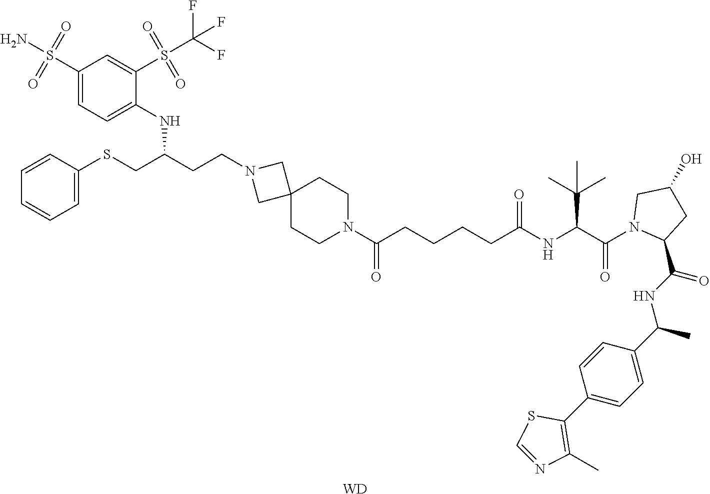 [SMILES: Cc1ncsc1-c1ccc([C@H](C)NC(=O)[C@@H]2C[C@@H](O)CN2C(=O)[C@@H](NC(=O)CCCCC(=O)N2CCC3(CC2)CN(CC[C@H](CSc2ccccc2)Nc2ccc(S(N)(=O)=O)cc2S(=O)(=O)C(F)(F)F)C3)C(C)(C)C)cc1 ; "WD"]

### Step 1—(9H-fluoren-9-yl)methyl ((R)-4-(7-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoyl)-2,7-diazaspiro[3.5]nonen-2-yl)-1-(phenylthio)butan-2-yl)carbamate

A solution of (2S,4R)-1-((S)-3,3-dimethyl-2-(6-oxo-6-(2,7-diazaspiro[3.5]nonan-7-yl) hexanamido)butanoyl)-4-hydroxy-N—((S)-1-(4-(4-methylthiazol-5-yl)phenyl)ethyl)pyrrolidine-2-carboxamide (250 mg, 367 μmol Intermediate WC), (9H-fluoren-9-yl)methyl(S)-(4-oxo-1-(phenylthio)butan-2-yl)carbamate (230 mg, 551 μmol, synthesized via Steps 1-3 of Intermediate KK from BC2-228), and NaBH₃CN (69.2 mg, 1.10 mmol) in DCM (1.5 mL) and MeOH (1.5 mL) was stirred at 25° C. for 2 hrs. On completion, the reaction mixture was quenched with sat. NH₄Cl (5 mL) and extracted with dichloromethane (5 mL×3). The combined organic layers were washed with brine (10 mL) and dried over anhydrous sodium sulfate, filtered and the filtrate was concentrated in vacuo to give the crude residue. The crude residue was purified by reversed-phase HPLC (0.8 g/L ammonium bicarbonate) to give the title compound (200 mg, 49% yield) as a white solid. LC-MS (ESI+) m/z 1083.5 (M+H)⁺.

### Step 2—(2S,4R)-1-((S)-2-(6-(2-((R)-3-amino-4-(phenylthio)butyl)-2,7-diazaspiro[3,5]nonan-7-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide

A solution of (9H-fluoren-9-yl)methyl ((R)-4-(7-(6-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-6-oxohexanoyl)-2,7-diazaspiro[3.5]nonan-2-yl)-1-(phenylthio)butan-2-yl)carbamate (150 mg, 138 μmol) and piperidine (118 mg, 1.39 mmol, 137 uL) in DMF (2 mL) was stirred at 0° C. for 2 hrs. On completion, the crude residue was purified by reversed-phase HPLC (0.8 g/L ammonium bicarbonate) to give the title compound (80.0 mg, 66% yield) as a white solid. LC-MS (ESI⁺) m/z 860.5 (M+H)⁺.

### Step 3—(2S,4R)-1-((S)-3,3-dimethyl-2-(6-oxo-6-(2-((R)-4-(phenylthio)-3-((4-sulfamoyl-2-((trifluoromethyl)sulfonyl)phenyl)amino)butyl)-2,7-diazaspiro[3.5]nonan-7-yl)hexanamido)butanoyl)-4-hydroxy-N—((S)-1-(4-(4-methylthiazol-5-yl)phenyl)ethyl)pyrrolidine-2-carboxamide

A solution of (2S,4R)-1-((S)-2-(6-(2-((R)-3-amino-4-(phenylthio)butyl)-2,7-diazaspiro[3.5]nonan-7-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (110 mg, 128 μmol), 4-fluoro-3-((trifluoromethyl)sulfonyl)benzenesulfonamide (39.3 mg, 128 μmol, CAS #1027345-08-9), and TEA (38.8 mg, 384 μmol, 53.4 uL) in THF (1.5 mL) was stirred at 50° C. for 2 hrs. On completion, the crude residue was purified by reversed-phase HPLC (0.8 g/L ammonium bicarbonate) to give the title compound (70.0 mg, 47% yield) as a white solid. LC-MS (ESI⁺) m/z 574.4 (½M+H)⁺.

### Synthesis of 8-(((S)-1-((2S,4R)-2-(((R)-1-(4-chlorophenyl)-2-hydroxyethyl)carbamoyl)-4-hydroxypyrrolidin-1-yl)-3,3-dimethyl-1-oxobutan-2-yl)amino)-8-oxooctanoic Acid (Intermediate WE)

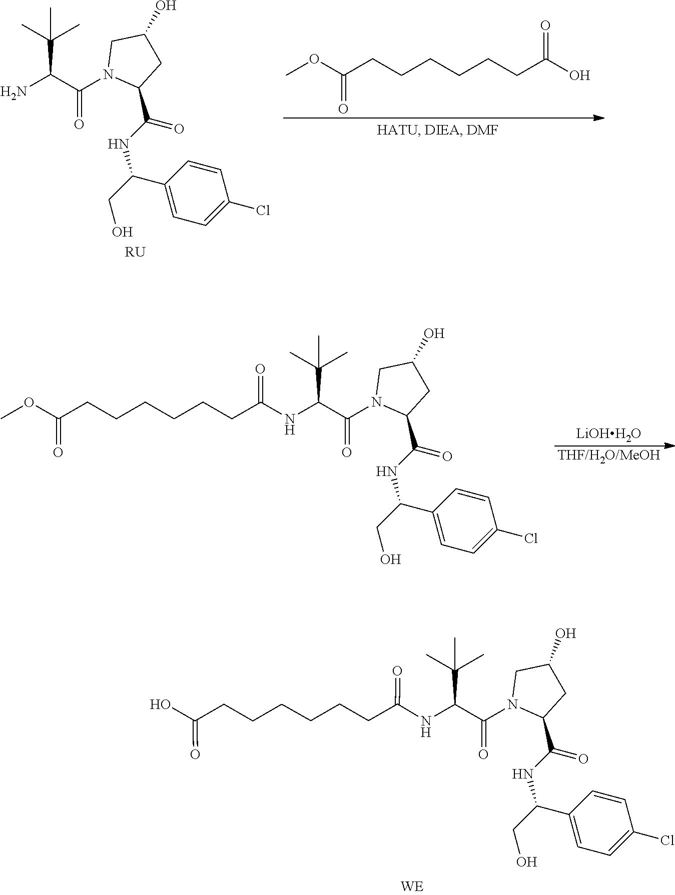

### Step 1—Methyl 8-(((S)-1-((2S,4R)-2-(((R)-1-(4-chlorophenyl)-2-hydroxyethyl)carbamoyl)-4-hydroxypyrrolidin-1-yl)-3,3-dimethyl-1-oxobutan-2-yl)amino)-8-oxooctanoate

To a solution of 8-methoxy-8-oxooctanoic acid (65.00 mg, 345 μmol, 61.9 uL, CAS #3946-32-5) in DMF (2 mL) was added HATU (170 mg, 448 μmol) and stirred for 5 min. Then DIEA (223 mg, 1.73 mmol, 300 uL) and (2S,4R)-1-((S)-2-amino-3,3-dimethylbutanoyl)-N—((R)-1-(4-chlorophenyl)-2-hydroxyethyl)-4-hydroxypyrrolidine-2-carboxamide (150 mg, 345 μmol. HCl, Intermediate RU) was added and the mixture was stirred at 25° C. for 10 min. On completion, the reaction mixture was concentrated under reduced pressure and purified by reversed-phase HPLC (0.1% FA condition) to give the title compound (166 mg, 85% yield) as a white solid. LC-MS (ESI⁺) m/z 568.4 (M+H)⁺.

### Step 2—8-(((S)-1-((2S,4R)-2-(((R)-1-(4-chlorophenyl)-2-hydroxyethyl)carbamoyl)-4-hydroxypyrrolidin-1-yl)-3,3-dimethyl-1-oxobutan-2-yl)amino)-8-oxooctanoic Acid

To solution of a methyl 8-(((S)-1-((2S,4R)-2-(((R)-1-(4-chlorophenyl)-2-hydroxyethyl)carbamoyl)-4-hydroxypyrrolidin-1-yl)-3,3-dimethyl-1-oxobutan-2-yl)amino)-8-oxooctanoate (166 mg, 292 μmol) in THF (4 mL), H₂O (1 mL) and MeOH (1 mL) was added LiOH·H₂O (61.3 mg, 1.46 mmol). The mixture was then stirred at 25° C. for 2 hrs. On completion, HCl (1N) was added to the reaction mixture until the pH was 6, then diluted with water (10 mL) and extracted with dichloromethane (3×10 mL). The combined organic layers were dried over anhydrous sodium sulfate, filtered and the filtrate was concentrated in vacuo to give the title compound (150 mg) as white solid. LC-MS (ESI⁺) m/z 555.2 (M+H)⁺.

### Synthesis of (2S,4R)-1-((S)-2-amino-3,3-dimethylbutanoyl)-4-hydroxy-N—((S)-1-(3-methoxy-1-methyl-1H-indazol-5-yl)ethyl)pyrrolidine-2-carboxamide (Intermediate WF)

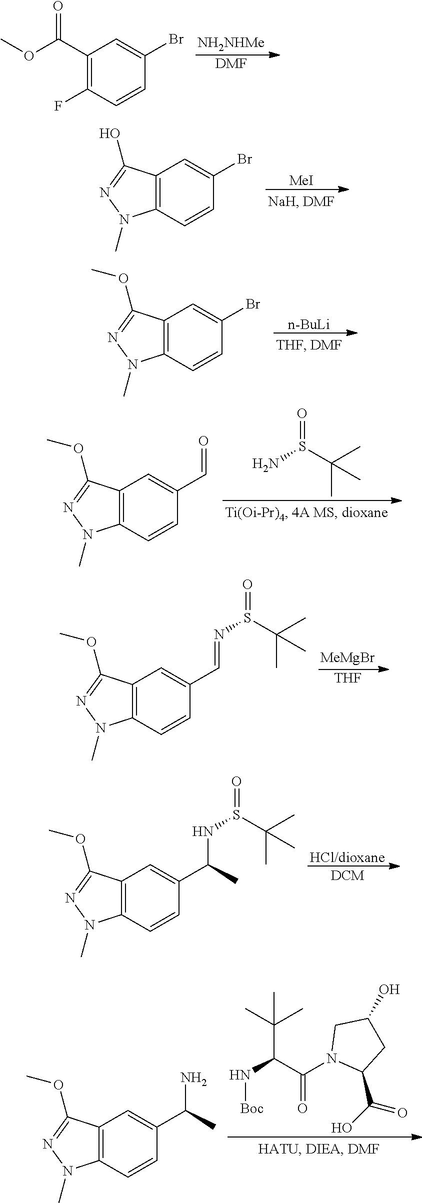

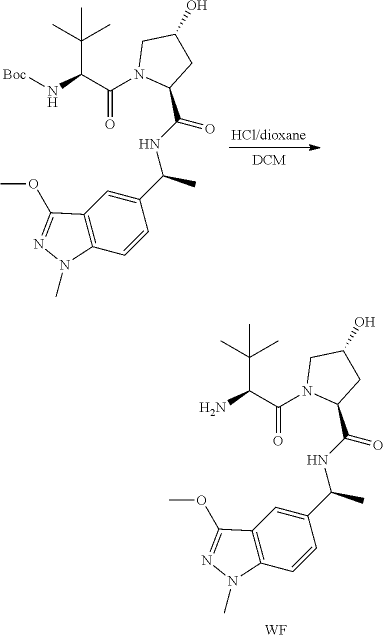

### Step 1—5-Bromo-1-methyl-1H-indazol-3-ol

To a solution of methyl 5-bromo-2-fluoro-benzoate (1.00 g, 4.29 mmol, CAS #57381-59-6) in DMA (10 mL) was added DIEA (582 mg, 4.51 mmol) and methylhydrazine (593 mg, 5.15 mmol, 40% solution) in sealed tube. The mixture was then stirred at 150° C. for 10 hrs. After cooling to room temperature, the reaction mixture was diluted with 30 mL of ethyl acetate, washed with H₂O (3×10 mL) and brine (1×20 mL) and concentrated in vacuo. The residue was purified by column chromatography (SiO₂, Petroleum ether/Ethyl acetate=10/1 to 1/1) to give the title compound (800 mg, 79% yield) as a yellow oil. LC-MS (ESI+) m/z 227.2 (M+H)⁺.

### Step 2—5-Bromo-3-methoxy-1-methyl-1H-indazole

To a solution of 5-bromo-1-methyl-indazol-3-ol (100 mg, 440 μmol) in THF (1 mL) was added NaH (21.1 mg, 528 μmol, 60% dispersion in mineral oil) at 0° C., and the mixture was stirred at 0° C. for 0.5 hrs. The mixture was then warmed to 25° C. and MeI (75.0 mg, 528 μmol) was added dropwise. Then the mixture was stirred at 25° C. for 2 hrs. On completion, the reaction mixture was quenched by addition of H₂O (5 mL) and then extracted with EA (5 mL×3). The combined organic layers were washed with brine (5 mL×3) and dried over Na₂SO₄, filtered and concentrated under reduced pressure to give a residue. The residue was purified by column chromatography (SiO₂, Petroleum ether/Ethyl acetate=20/1 to 10/1) to give the title compound (50 mg, 46% yield) as a yellow oil. LC-MS (ESI+) m/z 241.0 (M+H)⁺; ¹H NMR (400 MHz, CDCl₃-d) δ ppm 3.86 (s, 3H) 4.07 (s, 3H) 7.07-7.12 (m, 1H) 7.43 (dd, J=8.8, 1.6 Hz, 1H) 7.78 (d, J=1.2 Hz, 1H).

### Step 3—3-Methoxy-1-methyl-1H-indazole-5-carbaldehyde

To a solution of 5-bromo-3-methoxy-1-methyl-indazole (3 g, 12.4 mmol) in THF (50 mL) was added n-BuLi (2.5 M, 7.47 mL) at −75° C. The mixture was stirred at −75° C. for 1 hour and then warmed to −30° C., and stirred for 0.5 hr. Next, DMF (4.55 g, 62.2 mmol, 4.79 mL) was added over 0.5 hr. The reaction mixture was then quenched by addition of H₂O (30 mL) at 0° C., and then extracted with EA (30 mL×3). The combined organic layers were washed with brine (30 mL×3), dried over Na₂SO₄, filtered and concentrated under reduced pressure to give the title compound (2.5 g) as a yellow solid. LC-MS (ESI⁺) m/z 191.4 (M+H)⁺.

### Step 4—(R,E)-N-((3-methoxy-1-methyl-1H-indazol-5-yl)methylene)-2-methylpropane-2-sulfinamide

To a solution of 3-methoxy-1-methyl-indazole-5-carbaldehyde (2.30 g, 12.1 mmol) in DCM (25 mL) was added Ti(i-PrO)₄ (10.3 g, 36.3 mmol, 10.7 mL) and (R)-2-methylpropane-2-sulfinamide (1.47 g, 12.1 mmol, CAS #196929-78-9). The mixture was then stirred at 25° C. for 12 hrs. On completion, the reaction mixture was quenched by addition of H₂O (30 mL) at 0° C., and then extracted with DCM (30 mL×3). The combined organic layers were washed with brine (30 mL×3), dried over Na₂SO₄, filtered and the filtrate was concentrated under reduced pressure to give a residue. The residue was purified by column chromatography (SiO₂, Petroleum ether/Ethyl acetate=50/1 to 5/1) to give the title compound (2.4 g, 66% yield) as a white solid. LC-MS (ESI⁺) m/z 294.2 (M+H)⁺.

### Step 5—(R)—N—((S)-1-(3-methoxy-1-methyl-1H-indazol-5-yl)ethyl)-2-methylpropane-2-sulfinamide

To a solution of (NE,R)—N-[(3-methoxy-1-methyl-indazol-5-yl)methylene]-2-methyl-propane-2-sulfinamide (2.00 g, 6.82 mmol) in THF (30 mL) was added MeMgCl (3 M, 3.41 mL) under N₂ atmosphere at −50° C. The mixture was then stirred at −50° C. for 3 hrs. On completion, the reaction mixture was diluted by aq. NH₄Cl (3 mL) at 0° C. and extracted by ethyl acetate (3×10 mL). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether/Ethyl acetate=10/1 to 3/1) to give the title compound (1.00 g, 47% yield) a white solid. LC-MS (ESI⁺) m/z 310.0 (M+H)⁺.

### Step 6—(S)-1-(3-methoxy-1-methyl-1H-indazol-5-yl)ethan-1-amine

To a solution of (R)—N-[(1S)-1-(3-methoxy-1-methyl-indazol-5-yl)ethyl]-2-methyl-propane-2-sulfinamide (1.00 g, 3.23 mmol) in DCM (3 mL) was added HCl/dioxane (4 M, 800 uL). The mixture was then stirred at 25° C. for 2 hrs. On completion, the reaction mixture was concentrated in vacuo to give the title product (500 mg, HCl) as a yellow solid. LC-MS (ESI⁺) m/z 206.2 (M+H)⁺.

### Step 7—Tert-butyl ((S)-1-((2S,4R)-4-hydroxy-2-(((S)-1-(3-methoxy-1-methyl-1H-indazol-5-yl)ethyl)carbamoyl)pyrrolidin-1-yl)-3,3-dimethyl-1-oxobutan-2-yl)carbamate

A solution of (S)-1-(3-methoxy-1-methyl-1H-indazol-5-yl)ethan-1-amine (130 mg, 633 μmol), (2S,4R)-1-((S)-2-((tert-butoxycarbonyl)amino)-3,3-dimethylbutanoyl)-4-hydroxypyrrolidine-2-carboxylic acid (262 mg, 760 μmol, CAS #630421-46-4), HATU (361 mg, 950 μmol), and DIEA (245 mg, 1.90 mmol, 331 uL) in DMF (2 mL) was stirred at 0° C. for 10 min. On completion, the reaction mixture was quenched with water (10 mL) and extracted with ethyl acetate (10 mL×3). The combined organic layers were washed with brine (10 mL) and dried over anhydrous sodium sulfate, filtered and the filtrate was concentrated in vacuo to give the crude residue. The residue was purified by column chromatography (SiO₂, Petroleum ether:Ethyl acetate=1/1 to 1/2) to give the title compound (250 mg, 72% yield) as a yellow solid. LC-MS (ESI⁺) m/z 532.1 (M+H)⁺.

### Step 8—(2S,4R)-1-((S)-2-amino-3,3-dimethylbutanoyl)-4-hydroxy-N—((S)-1-(3-methoxy-1-methyl-1H-indazol-5-yl)ethyl)pyrrolidine-2-carboxamide

A solution of tert-butyl ((S)-1-((2S,4R)-4-hydroxy-2-(((S)-1-(3-methoxy-1-methyl-1H-indazol-5-yl)ethyl)carbamoyl)pyrrolidin-1-yl)-3,3-dimethyl-1-oxobutan-2-yl)carbamate (250 mg, 470 μmol) in HCl/dioxane (1 mL) and DCM (1 mL) was stirred at 25° C. for 12 hrs. On completion, the residue was concentrated under vacuum to give the title compound (200 mg) as a yellow solid. LC-MS (ESI⁺) m/z 432.5 (M+H)⁺.

### Synthesis of 8-(((S)-1-((2S,4R)-4-hydroxy-2-(((S)-1-(3-methoxy-1-methyl-1H-indazol-5-yl)ethyl)carbamoyl)pyrrolidin-1-yl)-3,3-dimethyl-1-oxobutan-2-yl)amino)-8-oxooctanoic Acid (Intermediate WG)

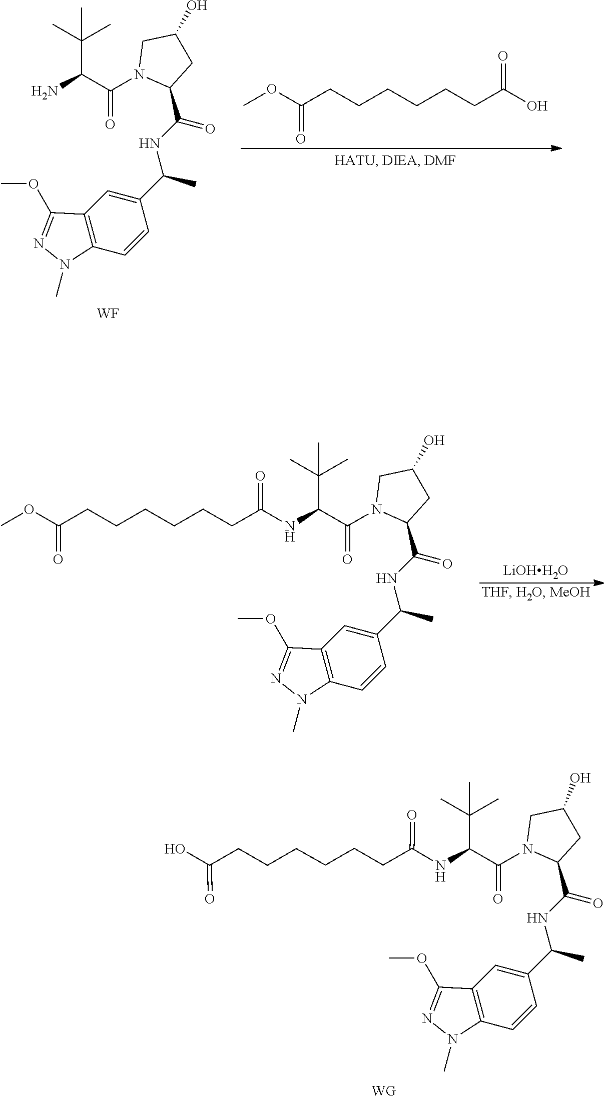

### Step 1—Methyl 8-(((S)-1-((2S,4R)-4-hydroxy-2-(((S)-1-(3-methoxy-1-methyl-1H-indazol-5-yl)ethyl)carbamoyl)pyrrolidin-1-yl)-3,3-dimethyl-1-oxobutan-2-yl)amino)-8-oxooctanoate

A solution of (2S,4R)-1-((S)-2-amino-3,3-dimethylbutanoyl)-4-hydroxy-N—((S)-1-(3-methoxy-1-methyl-1H-indazol-5-yl)ethyl)pyrrolidine-2-carboxamide (200 mg, 463 μmol, Intermediate WF), 8-methoxy-8-oxooctanoic acid (131 mg, 695 μmol, CAS #3946-32-5), HATU (264 mg, 695 μmol), and DIEA (180 mg, 1.39 mmol, 242 uL) in DMF (2 mL) was stirred at 0° C. for 10 min. On completion, the reaction mixture was quenched with water (10 mL) and extracted with ethyl acetate (10 mL×3). The combined organic layers were washed with brine (20 mL) and dried over anhydrous sodium sulfate, filtered and the filtrate was concentrated in vacuo to give the crude residue. The residue was purified by column chromatography (SiO₂, Petroleum ether:Ethyl acetate=1/1 to Dichloromethane:Methanol=10/1) to give the title compound (300 mg, 81% yield) as a yellow solid. LC-MS (ESI⁺) m/z 602.4 (M+H)⁺.

### Step 2—8-(((S)-1-((2S,4R)-4-hydroxy-2-(((S)-1-(3-methoxy-1-methyl-1H-indazol-5-yl)ethyl)carbamoyl)pyrrolidin-1-yl)-3,3-dimethyl-1-oxobutan-2-yl)amino)-8-oxooctanoic Acid

A solution of methyl 8-(((S)-1-((2S,4R)-4-hydroxy-2-(((S)-1-(3-methoxy-1-methyl-1H-indazol-5-yl)ethyl)carbamoyl)pyrrolidin-1-yl)-3,3-dimethyl-1-oxobutan-2-yl)amino)-8-oxooctanoate (200 mg, 332 μmol) and LiOH·H₂O (69.7 mg, 1.66 mmol) in MeOH (1 mL), THF (1 mL), and H₂O (1 mL) was stirred at 50° C. for 2 hrs. On completion, the crude residue was purified by reversed-phase HPLC (0.8 g/L ammonium bicarbonate) to give the title compound (150 mg, 75% yield) as a white solid. LC-MS (ESI⁺) m/z 588.3 (M+H)⁺.

### Synthesis of (R)—N—((S)-1-(4-bromo-2-(methoxymethoxy)phenyl)ethyl)-2-methylpropane-2-sulfinamide (Intermediate WH)

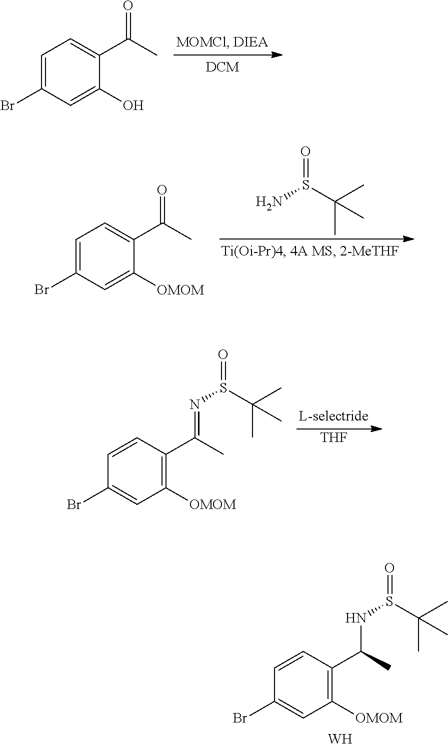

### Step 1—1-(4-Bromo-2-(methoxymethoxy)phenyl)ethan-1-one

To a solution of 1-(4-bromo-2-hydroxy-phenyl)ethanone (38 g, 176 mmol) in DMF (400 mL) was added bromo(methoxy)methane (44.2 g, 353 mmol, 28.8 mL) and K₂CO₃ (97.7 g, 706 mmol). The mixture was degassed and purged with N₂ three times, and then the mixture was stirred at 0-25° C. for 4 hr under N₂ atmosphere. On completion, the reaction mixture was quenched by addition of Na₂CO₃ (50 mL) at 25° C., and then diluted with H₂O (300 mL) and extracted with EA (200 mL×3). The combined organic layers were washed with aqueous NaCl (200 mL×3), dried over Na₂SO₄, filtered and concentrated under reduced pressure to give a residue. The residue was purified by column chromatography (SiO₂, Petroleum ether/Ethyl acetate=10/0 to 10/1) to give the title compound (33 g, 72% yield) as a colorless oil. ¹H NMR (400 MHz, DMSO-d₆) δ ppm 7.48 (d, J=8.4 Hz, 1H), 7.27 (d, J=1.6 Hz, 1H), 7.06 (dd, J=8.0, 1.6 Hz, 1H), 5.18 (s, 2H), 3.15 (s, 3H), 2.50 (s, 3H).

### Step 2—(R,E)-N-(1-(4-bromo-2-(methoxymethoxy)phenyl)ethylidene)-2-methylpropane-2-sulfinamide

To a solution of 1-[4-bromo-2-(methoxymethoxy)phenyl]ethanone (30 g, 115 mmol) and (R)-2-methylpropane-2-sulfinamide (56.1 g, 463 mmol) in 2-MeTHF (300 mL) was added Ti(i-PrO)₄ (98.7 g, 347 mmol, 102 mL) and 4 Å molecular sieves (30 g) at 0° C. under N₂. The mixture was stirred at 100° C. for 12 hr. On completion, the reaction mixture was added to EA (1500 mL), and then was added H₂O (1000 mL) and the mixture was stirred at 20° C. for 10 min. This resulted in a solid/liquid mixture which was filtered under reduced pressure to give a filtrate. The filtrate was extracted with EA (1000 mL×2). The combined organic layers were dried over Na₂SO₄, filtered and concentrated under reduced pressure to give a residue. The residue was purified by column chromatography (SiO₂, Petroleum ether/Ethyl acetate=10/0 to 9/1) to give the title compound (35 g, 76% yield) LC-MS (ESI+) m/z 361.8 (M+H)⁺.

### Step 3—(R)—N—((S)-1-(4-bromo-2-(methoxymethoxy)phenyl)ethyl)-2-methylpropane-2-sulfinamide

To a solution of (NE,R)—N-[1-[4-bromo-2-(methoxymethoxy)phenyl]ethylidene]-2-methyl-propane-2-sulfinamide (35 g, 96.6 mmol) in THF (400 mL) was added L-selectride (1 M, 145 mL) at 0° C. under N₂. The mixture was stirred at 0-20° C. for 2 hr. On completion, the reaction mixture was quenched by addition of NH₄Cl (200 mL) at 0° C., and then diluted with H₂O (300 mL) and extracted with EA (500 mL×2). The combined organic layers were washed with aqueous NaCl (500 mL×2), dried over Na₂SO₄, filtered and concentrated under reduced pressure to give a residue (35 g) as a yellow oil. LC-MS (ESI+) m/z 363.8 (M+H)⁺.

### Example 2: Synthesis of Compounds of Formula I (Method 1)

### Synthesis of (2S,4R)-1-((S)-2-(7-(4-((R)-3-((4-(N-(4-(4-((R)-(4′-chloro-[1,1′-biphenyl]-2-yl)(hydroxy)methyl)piperidin-1-yl)benzoyl)sulfamoyl)-2-((trifluoromethyl)sulfonyl)phenyl)amino)-4-(phenylthio)butyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (I-28)

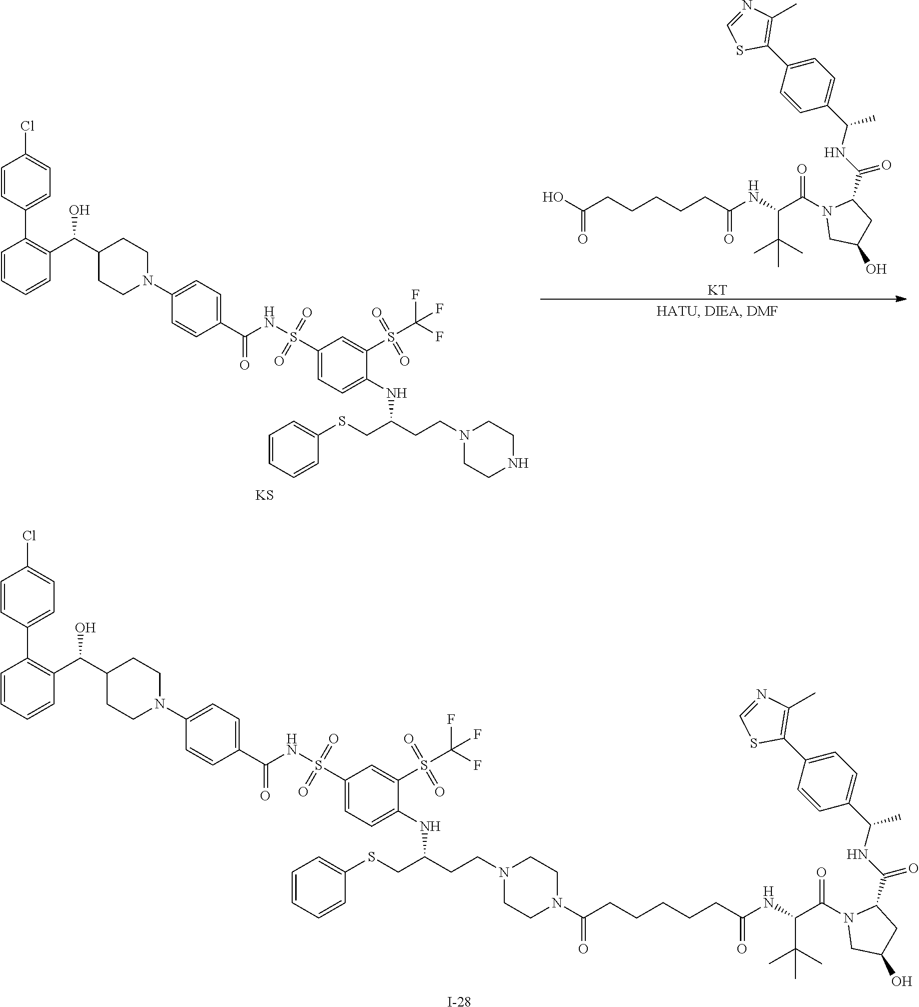

To a solution of) 7-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-7-oxo-heptanoic acid (70.9 mg, 120 μmol, Intermediate KT) in DMF (4 mL) was added DIEA (46.8 mg, 362 μmol) and HATU (55.1 mg, 145 μmol). Next, 4-[4-[(R)-[2-(4-chlorophenyl)phenyl]-hydroxy-methyl]-1-piperidyl]-N-[4-[[(1R)-1-(phenylsulfanylmethyl)-3-piperazin-1-yl-propyl]amino]-3-(trifluoromethylsulfonyl)phenyl]sulfonyl-benzamide (120 mg, 120 μmol, Intermediate KS) was added and the mixture was stirred at 25° C. for 16 hrs. On completion, the reaction mixture was quenched with H₂O (2 mL) at 25° C., and then diluted with DCM/MeOH (10/1, 20 mL) and extracted with DCM/MeOH (10/1, 30 mL×2). The combined organic layers were washed with aqueous NaCl (30 mL×2), dried over Na₂SO₄, filtered and concentrated under reduced pressure to give a residue. The crude product was purified by reversed-phase HPLC (column: Waters xbridge 150*25 mm 10 um; mobile phase: [water (NH4HCO3)-ACN]; B %: 40%-70%, 8 min) to give a residue (15 mg, 8% yield) as a white solid. LC-MS (ESI⁺) m/z 1524.0 (M+H)⁺; ¹H NMR (400 MHz, DMSO-d₆) δ=8.98 (s, 1H), 8.38-8.33 (m, 1H), 8.12 (s, 1H), 7.95 (d, J=9.2 Hz, 1H), 7.78 (d, J=9.2 Hz, 1H), 7.68-7.65 (m, 2H), 7.58 (d, J=7.6 Hz, 1H), 7.49-7.46 (m, 2H), 7.44-7.41 (m, 3H), 7.39-7.38 (m, 1H), 7.37-7.34 (m, 2H), 7.34-7.31 (m, 4H), 7.30-7.22 (m, 3H), 7.21-7.08 (m, 3H), 7.04-6.97 (m, 1H), 6.97-6.88 (m, 1H), 6.79 (d, J=8.8 Hz, 2H), 5.22 (d, J=4.4 Hz, 1H), 5.09 (d, J=3.2 Hz, 1H), 4.91 (t, J=7.2 Hz, 1H), 4.51 (d, J=9.6 Hz, 1H), 4.42 (t, J=8.0 Hz, 1H), 4.32-4.27 (m, 2H), 4.13-4.05 (m, 1H), 3.87-3.78 (m, 1H), 3.67-3.57 (m, 3H), 2.70-2.60 (m, 2H), 2.45 (s, 3H), 2.45-2.43 (m, 1H), 2.33 (s, 1H), 2.24-2.18 (m, 4H), 2.14-2.07 (m, 1H), 2.17-1.94 (m, 5H), 1.94-1.84 (m, 2H), 1.83-1.74 (m, 2H), 1.67-1.61 (m, 1H), 1.52-1.35 (m, 9H), 1.28-1.17 (m, 3H), 1.12-1.06 (m, 1H), 1.05-0.95 (m, 2H), 0.93 (s, 9H), 0.90-0.76 (m, 2H).

TABLE 3
Compounds synthesized via Method 1, using the corresponding
amines and acids for the coupling.

| | | | LCMS | |
|---|---|---|---|---|
| | | | (ESI+) | |
| | | | m/z | |
| I-#^{a} | Amine | Acid | (M + H)⁺ | ¹H NMR (400 MHz, DMSO-d6) |
| I-5 | LL | LG | 1493.6 | 9.00-8.94 (m, 1H), 8.46 (d, J = 2.4 Hz, |
| | | | | 1H), 8.39-8.33 (m, 1H), 8.14 (s, 1H), |
| | | | | 7.84-7.75 (m, 2H), 7.74-7.70 (m, 2H), |
| | | | | 7.47 (s, 4H), 7.45-7.41 (m, 3H), 7.39- |
| | | | | 7.36 (m, 2H), 7.32-7.27 (m, 3H), 7.27- |
| | | | | 7.21 (m, 3H), 7.19-7.15 (m, 1H), 6.95- |
| | | | | 6.87 (m, 1H), 6.82-6.77 (m, 2H), 5.13- |
| | | | | 5.06 (m, 1H), 4.97-4.87 (m, 1H), 4.54- |
| | | | | 4.48 (m, 1H), 4.46-4.39 (m, 1H), 4.31- |
| | | | | 4.22 (m, 1H), 4.12-4.01 (m, 1H), 3.61- |
| | | | | 3.53 (m, 4H), 3.48-3.42 (m, 5H), 3.38 |
| | | | | (d, J = 4.0 Hz, 8H), 3.17-3.11 (m, 4H), |
| | | | | 2.60 (d, J = 2.4 Hz, 4H), 2.45 (s, 3H), |
| | | | | 2.39 (s, 6H), 2.29-2.20 (m, 4H), 2.13- |
| | | | | 2.07 (m, 2H), 2.04-1.96 (m, 2H), 1.82- |
| | | | | 1.76 (m, 1H), 1.51-1.40 (m, 5H), 1.39- |
| | | | | 1.35 (m, 3H), 1.27-1.22 (m, 4H), 0.93 |
| | | | | (s, 9H) |
| I-6 | LJ | LG | 1509.1 | 8.99-8.97 (m, 1H), 8.46-8.43 (m, 1H), |
| | | | | 8.39-8.35 (m, 1H), 8.29-8.17 (m, 1H), |
| | | | | 7.81-7.76 (m, 2H), 7.72 (d, J = 8.8 Hz, |
| | | | | 2H), 7.56 (s, 1H), 7.50 (s, 4H), 7.44- |
| | | | | 7.41 (m, 3H), 7.40-7.36 (m, 3H), 7.35- |
| | | | | 7.28 (m, 4H), 7.24 (t, J = 7.6 Hz, 2H), |
| | | | | 7.19-7.15 (m, 1H), 6.88 (br d, J = 9.2 |
| | | | | Hz, 1H), 6.79 (br d, J = 8.8 Hz, 2H), |
| | | | | 5.11-5.08 (m, 1H), 4.94-4.89 (m, 1H), 4.51 |
| | | | | (d, J = 9.2 Hz, 1H), 4.42 (t, J = 8.4 Hz, |
| | | | | 1H), 4.27 (br s, 1H), 4.09-4.03 (m, 1H), |
| | | | | 3.60 (br s, 3H), 3.52 (ddd, J = 5.6, 9.2, |
| | | | | 12.0 Hz, 5H), 3.42 (br s, 4H), 3.14 (br s, |
| | | | | 5H), 2.45 (s, 3H), 2.41 (br s, 5H), 2.25 |
| | | | | (br s, 2H), 2.14-2.06 (m, 2H), 2.05- |
| | | | | 1.97 (m, 3H), 1.81-1.75 (m, 1H), 1.56- |
| | | | | 1.41 (m, 6H), 1.37 (d, J = 7.2 Hz, 3H), |
| | | | | 1.30-1.19 (m, 7H), 0.93 (s, 12H) |
| I-7 | LL | KT | 1479.6 | 9.00-8.92 (m, 1H), 8.49-8.41 (m, 1H), |
| | | | | 8.37 (d, J = 7.6 Hz, 1H), 8.25-8.09 (m, |
| | | | | 1H), 7.83-7.77 (m, 2H), 7.72 (d, J = 8.8 |
| | | | | Hz, 2H), 7.47 (s, 4H), 7.45-7.41 (m, |
| | | | | 3H), 7.39-7.36 (m, 2H), 7.30 (t, J = 6.8 |
| | | | | Hz, 3H), 7.26-7.20 (m, 3H), 7.18-7.14 |
| | | | | (m, 1H), 6.93-6.88 (m, 1H), 6.80 (d, J = |
| | | | | 8.8 Hz, 2H), 5.10 (d, J = 3.6 Hz, 1H), |
| | | | | 4.96-4.88 (m, 1H), 4.54-4.48 (m, 1H), |
| | | | | 4.45-4.39 (m, 1H), 4.30-4.25 (m, 1H), |
| | | | | 4.12-4.03 (m, 1H), 3.62-3.53 (m, 4H), |
| | | | | 3.44 (s, 5H), 3.36 (d, J = 16.0 Hz, 8H), |
| | | | | 3.14 (s, 4H), 2.62 (s, 4H), 2.45 (s, 3H), |
| | | | | 2.39 (s, 6H), 2.29-2.19 (m, 4H), 2.14- |
| | | | | 2.08 (m, 2H), 2.05-1.97 (m, 2H), 1.82- |
| | | | | 1.76 (m, 1H), 1.53-1.42 (m, 5H), 1.37 |
| | | | | (d, J = 7.2 Hz, 3H), 1.27-1.21 (m, 2H), |
| | | | | 0.93 (s, 9H) |
| I-8 | LL | LP | 1507.6 | 9.02-8.95 (m, 1H), 8.48-8.43 (m, 1H), |
| | | | | 8.39-8.33 (m, 1H), 8.25-8.14 (m, 1H), |
| | | | | 7.76 (s, 2H), 7.72 (d, J = 8.8 Hz, 2H), |
| | | | | 7.47 (s, 4H), 7.45-7.41 (m, 3H), 7.39- |
| | | | | 7.36 (m, 2H), 7.30 (t, J = 7.2 Hz, 3H), |
| | | | | 7.26-7.21 (m, 3H), 7.20-7.16 (m, 1H), |
| | | | | 6.95-6.88 (m, 1H), 6.82-6.77 (m, 2H), |
| | | | | 5.05 (s, 1H), 4.97-4.87 (m, 1H), 4.55- |
| | | | | 4.48 (m, 1H), 4.46-4.38 (m, 1H), 4.33- |
| | | | | 4.23 (m, 1H), 4.13-4.02 (m, 1H), 3.61- |
| | | | | 3.53 (m, 4H), 3.44 (s, 5H), 3.36-3.36 |
| | | | | (m, 1H), 3.38 (d, J = 5.2 Hz, 7H), 3.14 |
| | | | | (d, J = 1.6 Hz, 4H), 2.63 (d, J = 8.4 Hz, |
| | | | | 4H), 2.45 (s, 3H), 2.39 (s, 6H), 2.29- |
| | | | | 2.20 (m, 4H), 2.14-2.08 (m, 2H), 2.05- |
| | | | | 1.97 (m, 2H), 1.81-1.75 (m, 1H), 1.52- |
| | | | | 1.41 (m, 5H), 1.37 (d, J = 7.2 Hz, 3H), |
| | | | | 1.24 (s, 6H), 0.93 (s, 9H) |
| I-11 | LU | LT | 1411.6 | 8.94-9.02 (m, 1H), 8.50 (br t, J = 5.6 |
| | | | | Hz, 1H), 8.44-8.48 (m, 1H), 8.37 (d, J = |
| | | | | 7.6 Hz, 1H), 8.16-8.26 (m, 1H), 7.96 |
| | | | | (d, J = 1.2 Hz, 1H), 7.82-7.85 (m, 1H), |
| | | | | 7.77-7.82 (m, 2H), 7.72 (d, J = 8.8 Hz, |
| | | | | 2H), 7.51 (s, 4H), 7.36-7.47 (m, 5H), |
| | | | | 7.21-7.35 (m, 6H), 7.14-7.19 (m, 1H), |
| | | | | 6.91 (br d, J = 9.2 Hz, 1H), 6.80 (br d, |
| | | | | J = 9.2 Hz, 2H), 5.06-5.14 (m, 1H), 4.88- |
| | | | | 4.96 (m, 1H), 4.52 (d, J = 9.2 Hz, 1H), |
| | | | | 4.40-4.47 (m, 1H), 4.25-4.32 (m, 1H), |
| | | | | 4.02-4.12 (m, 1H), 3.58-3.63 (m, 2H), |
| | | | | 3.42 (br s, 3H), 3.14 (br s, 4H), 2.83- |
| | | | | 3.04 (m, 3H), 2.59 (br s, 3H), 2.46 (s, |
| | | | | 3H), 2.40 (br s, 4H), 2.19-2.32 (m, 2H), |
| | | | | 1.98-2.18 (m, 5H), 1.76-1.83 (m, 1H), |
| | | | | 1.42-1.60 (m, 6H), 1.37 (d, J = 7.2 Hz, |
| | | | | 3H), 1.26-1.34 (m, 4H), 0.92-0.96 (m, |
| | | | | 9H) |
| I-12 | LZ | LG | 1436.5 | 8.98 (s, 1H), 8.50 (s, 1H), 8.37 (d, J = 7.6 |
| | | | | Hz, 1H), 7.81-7.70 (m, 4H), 7.53-7.50 |
| | | | | (m, 1H), 7.47 (s, 4H), 7.45-7.40 (m, |
| | | | | 3H), 7.39-7.36 (m, 3H), 7.30-7.18 (m, |
| | | | | 6H), 7.15 (d, J = 7.2 Hz, 1H), 7.10-6.99 |
| | | | | (m, 1H), 6.86 (d, J = 8.4 Hz, 2H), 5.09 |
| | | | | (d, J = 3.6 Hz, 1H), 4.91 (s, 1H), 4.51 (d, |
| | | | | J = 9.2 Hz, 1H), 4.45-4.39 (m, 1H), 4.27 |
| | | | | (s, 1H), 4.20-4.10 (m, 1H), 3.59 (s, 2H), |
| | | | | 3.39 (s, 4H), 3.21 (s, 6H), 2.67 (td, J = |
| | | | | 1.6, 3.6 Hz, 1H), 2.45 (s, 3H), 2.39 (s, |
| | | | | 6H), 2.34-2.31 (m, 2H), 2.21 (t, J = 7.6 |
| | | | | Hz, 4H), 2.15-2.06 (m, 2H), 2.00 (s, |
| | | | | 3H), 1.79 (s, 3H), 1.48-1.41 (m, 4H), |
| | | | | 1.37 (d, J = 7.2 Hz, 3H), 1.23 (s, 5H), |
| | | | | 0.93 (s, 9H) |
| I-17 | MP | MK | 1533.6 | 8.98 (s, 1H), 8.44 (d, J = 1.6 Hz, 1H), |
| | | | | 8.36 (d, J = 8.4 Hz, 1H), 8.30-8.21 (m, |
| | | | | 1H), 7.79 (d, J = 9.2 Hz, 2H), 7.72 (br d, |
| | | | | J = 8.4 Hz, 2H), 7.47 (s, 5H), 7.44-7.41 |
| | | | | (m, 2H), 7.39-7.35 (m, 2H), 7.34-7.28 |
| | | | | (m, 3H), 7.24 (t, J = 7.2 Hz, 3H), 7.19- |
| | | | | 7.14 (m, 1H), 6.87 (d, J = 9.2 Hz, 1H), |
| | | | | 6.79 (d, J = 8.4 Hz, 2H), 5.10 (d, J = 3.2 |
| | | | | Hz, 1H), 4.96-4.86 (m, 1H), 4.56-4.48 |
| | | | | (m, 1H), 4.46-4.38 (m, 1H), 4.32-4.23 |
| | | | | (m, 1H), 4.10-3.99 (m, 1H), 3.63-3.56 |
| | | | | (m, 3H), 3.39 (s, 8H), 3.14 (s, 3H), 2.45 |
| | | | | (s, 6H), 2.43-2.41 (m, 2H), 2.40 (s, 3H), |
| | | | | 2.38-2.35 (m, 2H), 2.30-2.23 (m, 4H), |
| | | | | 2.17-2.08 (m, 2H), 2.07-1.92 (m, 4H), |
| | | | | 1.87-1.70 (m, 2H), 1.54-1.44 (m, 8H), |
| | | | | 1.40 (d, J = 2.0 Hz, 2H), 1.37 (d, J = 7.2 |
| | | | | Hz, 4H), 1.33 (s, 2H), 1.23 (s, 2H), 0.93 |
| | | | | (s, 9H) |
| I-18 | LL | F | 1553.6 | 9.00-8.94 (m, 1H), 8.49 (t, J = 6.0 Hz, |
| | | | | 1H), 8.46 (s, 1H), 8.24-8.11 (m, 1H), |
| | | | | 7.82 (d, J = 8.4 Hz, 1H), 7.72 (d, J = 8.4 |
| | | | | Hz, 2H), 7.51-7.46 (m, 4H), 7.44 (s, |
| | | | | 1H), 7.40 (d, J = 7.6 Hz, 1H), 7.29 (t, J = |
| | | | | 8.0 Hz, 4H), 7.26-7.16 (m, 4H), 6.99 (s, |
| | | | | 1H), 6.97-6.89 (m, 2H), 6.80 (d, J = 8.4 |
| | | | | Hz, 2H), 5.19-5.12 (m, 1H), 4.63-4.56 |
| | | | | (m, 1H), 4.55-4.46 (m, 1H), 4.38-4.33 |
| | | | | (m, 1H), 4.32-4.25 (m, 1H), 4.23-4.15 |
| | | | | (m, 1H), 4.12-4.02 (m, 3H), 3.67-3.58 |
| | | | | (m, 2H), 3.53 (s, 2H), 3.45 (s, 5H), 3.38 |
| | | | | (s, 5H), 3.19-3.11 (m, 4H), 3.06-2.90 |
| | | | | (m, 2H), 2.63 (s, 4H), 2.45 (s, 3H), 2.39 |
| | | | | (s, 6H), 2.33-2.25 (m, 4H), 2.13-2.02 |
| | | | | (m, 3H), 1.95-1.88 (m, 1H), 1.78-1.70 |
| | | | | (m, 2H), 1.58-1.40 (m, 5H), 1.39-1.30 |
| | | | | (m, 4H), 1.24-1.19 (m, 2H), 0.98-0.92 |
| | | | | (m, 9H) |
| I-20 | NN | MZ | 1452.6 | 8.99 (s, 1H), 8.47 (d, J = 2.0 Hz, 1H), |
| | | | | 8.32-8.40 (m, 2H), 8.20 (d, J = 8.8 Hz, |
| | | | | 1H), 7.80-7.86 (m, 1H), 7.76-7.80 (m, |
| | | | | 1H), 7.71-7.76 (m, 2H), 7.42-7.49 (m, |
| | | | | 6H), 7.36-7.40 (m, 3H), 7.29-7.33 (m, |
| | | | | 2H), 7.16-7.27 (m, 5H), 6.92 (d, J = 9.2 |
| | | | | Hz, 1H), 6.80 (d, J = 8.8 Hz, 2H), 5.10 |
| | | | | (d, J = 3.2 Hz, 1H), 4.92 (q, J = 7.2 Hz, |
| | | | | 1H), 4.52 (d, J = 9.2 Hz, 1H), 4.43 (t, |
| | | | | J = 8.0 Hz, 1H), 4.30 (d, J = 6.0 Hz, 3H), |
| | | | | 4.02-4.13 (m, 1H), 3.56-3.67 (m, 2H), |
| | | | | 3.35-3.39 (m, 4H), 3.14 (s, 4H), 2.90- |
| | | | | 3.06 (m, 2H), 2.62 (s, 6H), 2.46 (s, 3H), |
| | | | | 2.40 (s, 4H), 2.19-2.27 (m, 1H), 2.00- |
| | | | | 2.16 (m, 6H), 1.80 (ddd, J = 12.8, 8.4, |
| | | | | 4.4 Hz, 1H), 1.44-1.56 (m, 4H), 1.37 (d, |
| | | | | J = 7.2 Hz, 3H), 1.23 (s, 9H), 0.93 (s, 9H) |
| I-23 | MH | KT | 1534.4 | 11.73-11.64 (m, 1H), 8.98 (s, 1H), 8.40- |
| | | | | 8.34 (m, 1H), 8.17-8.11 (m, 1H), 8.03- |
| | | | | 7.98 (m, 1H), 7.94-7.86 (m, 1H), 7.82- |
| | | | | 7.77 (m, 1H), 7.54-7.49 (m, 3H), 7.47- |
| | | | | 7.44 (m, 4H), 7.42 (s, 2H), 7.39-7.36 |
| | | | | (m, 3H), 7.32-7.25 (m, 4H), 7.10 (d, J = |
| | | | | 7.2 Hz, 1H), 7.05-6.97 (m, 1H), 6.64- |
| | | | | 6.58 (m, 1H), 6.39 (s, 1H), 6.11 (s, 1H), |
| | | | | 5.19 (d, J = 4.0 Hz, 1H), 5.09 (d, J = 3.6 |
| | | | | Hz, 1H), 4.96-4.89 (m, 1H), 4.51 (d, J = |
| | | | | 9.2 Hz, 1H), 4.42 (t, J = 8.0 Hz, 1H), |
| | | | | 4.30-4.21 (m, 2H), 3.60 (s, 2H), 3.44 (s, 3H), |
| | | | | 3.37 (d, J = 5.2 Hz, 2H), 3.29 (s, 2H), |
| | | | | 2.57 (s, 2H), 2.45 (s, 3H), 2.42-2.35 (m, |
| | | | | 4H), 2.28-2.22 (m, 4H), 2.11 (dd, J = |
| | | | | 7.2, 13.6 Hz, 1H), 2.04-1.97 (m, 1H), |
| | | | | 1.83-1.70 (m, 5H), 1.51-1.42 (m, 5H), |
| | | | | 1.37 (d, J = 6.8 Hz, 3H), 1.29-1.21 (m, |
| | | | | 3H), 0.99 (d, J = 10.8 Hz, 1H), 0.93 (s, |
| | | | | 12H), 0.78-0.70 (m, 1H) |
| I-24 | MV | KT | 1595.6 | 8.98 (s, 1H), 8.37 (d, J = 7.6 Hz, 1H), |
| | | | | 8.09 (d, J = 2.0 Hz, 1H), 8.00-7.94 (m, |
| | | | | 1H), 7.79 (d, J = 9.2 Hz, 1H), 7.67 (d, |
| | | | | J = 8.8 Hz, 2H), 7.58 (d, J = 7.6 Hz, IH), |
| | | | | 7.48 (d, J = 8.4 Hz, 2H), 7.45-7.41 (m, |
| | | | | 3H), 7.40-7.37 (m, 2H), 7.37-7.33 (m, |
| | | | | 5H), 7.31-7.26 (m, 3H), 7.21-7.17 (m, |
| | | | | 1H), 7.13 (d, J = 7.6 Hz, 1H), 6.90 (d, |
| | | | | J = 9.2 Hz, 1H), 6.77-6.70 (m, 3H), 5.22 |
| | | | | (d, J = 4.4 Hz, 1H), 5.10 (s, 1H), 4.96- |
| | | | | 4.88 (m, 1H), 4.51 (d, J = 9.2 Hz, 1H), |
| | | | | 4.42 (t, J = 8.0 Hz, 1H), 4.33-4.26 (m, |
| | | | | 2H), 4.02 (d, J = 5.2 Hz, 1H), 3.73 (d, J = |
| | | | | 12.4 Hz, 1H), 3.60 (s, 2H), 3.33 (dd, J = |
| | | | | 5.6, 14.0 Hz, 8H), 3.01-2.94 (m, 1H), |
| | | | | 2.89-2.81 (m, 2H), 2.61-2.54 (m, 4H), |
| | | | | 2.45 (s, 3H), 2.36-2.32 (m, 4H), 2.25 (t, |
| | | | | J = 7.2 Hz, 3H), 2.15-2.06 (m, 2H), 2.04- |
| | | | | 1.97 (m, 2H), 1.90 (d, J = 12.0 Hz, 1H), |
| | | | | 1.79 (ddd, J = 4.8, 8.4, 12.8 Hz, 1H), 1.68 |
| | | | | (d, J = 5.6 Hz, 2H), 1.61 (dd, J = 3.2, 7.2 |
| | | | | Hz, 1H), 1.52-1.41 (m, 5H), 1.37 (d, J = |
| | | | | 7.2 Hz, 3H), 1.29-1.19 (m, 3H), 1.13- |
| | | | | 1.07 (m, 1H), 1.00 (d, J = 12.8 Hz, 2H), |
| | | | | 0.93 (s, 9H), 0.87-0.81 (m, 1H) |
| I-25 | MY | LG | 1626.8 | 8.98 (s, 1H), 8.37 (d, J = 8.0 Hz, 1H), |
| | | | | 8.07 (s, 1H), 7.95 (d, J = 9.0 Hz, 1H), |
| | | | | 7.78 (d, J = 9.2 Hz, 1H), 7.66 (d, J = 8.4 |
| | | | | Hz, 2H), 7.52 (s, 1H), 7.48 (d, J = 8.4 Hz, |
| | | | | 2H), 7.44-7.42 (m, 2H), 7.38 (s, 1H), |
| | | | | 7.37-7.33 (m, 6H), 7.30 (d, J = 7.6 Hz, |
| | | | | 2H), 7.26 (d, J = 4.8 Hz, 1H), 7.22 (d, |
| | | | | J = 10.0 Hz, 1H), 7.21-7.17 (m, 1H), 7.10 |
| | | | | (d, J = 8.0 Hz, 1H), 6.87 (d, J = 9.2 Hz, |
| | | | | 1H), 6.71 (d, J = 8.8 Hz, 2H), 5.23 (d, |
| | | | | J = 4.0 Hz, 1H), 5.10 (d, J = 3.6 Hz, 1H), |
| | | | | 4.91 (t, J = 7.2 Hz, 1H), 4.51 (d, J = 9.2 |
| | | | | Hz, 1H), 4.42 (t, J = 7.6 Hz, 1H), 4.34- |
| | | | | 4.26 (m, 2H), 4.05-3.96 (m, 1H), 3.75- |
| | | | | 3.68 (m, 1H), 3.60 (s, 3H), 3.55 (s, 3H), |
| | | | | 3.45 (s, 4H), 3.28-3.22 (m, 3H), 2.60- |
| | | | | 2.55 (m, 2H), 2.45 (s, 3H), 2.43-2.37 |
| | | | | (m, 4H), 2.33 (d, J = 1.6 Hz, 4H), 2.29- |
| | | | | 2.19 (m, 5H), 2.14-2.06 (m, 2H), 2.04- |
| | | | | 1.98 (m, 2H), 1.91-1.83 (m, 2H), 1.82- |
| | | | | 1.73 (m, 2H), 1.60-1.54 (m, 1H), 1.48- |
| | | | | 1.43 (m, 4H), 1.37 (d, J = 7.2 Hz, 3H), |
| | | | | 1.24 (d, J = 6.4 Hz, 3H), 1.16-1.04 (m, |
| | | | | 2H), 1.03-0.96 (m, 2H), 0.93 (s, 9H) |
| I-27 | A | NJ | 1617.5 | 10.14-10.06 (m, 1H), 9.02-8.93 (m, |
| | | | | 1H), 8.43-8.36 (m, 1H), 8.21-8.16 (m, |
| | | | | 1H), 8.07 (s, 1H), 7.92 (dd, J = 8.4, 16.4 |
| | | | | Hz, 2H), 7.84-7.79 (m, 1H), 7.68-7.60 |
| | | | | (m, 4H), 7.53 (d, J = 8.4 Hz, 2H), 7.45- |
| | | | | 7.42 (m, 2H), 7.41-7.32 (m, 7H), 7.32- |
| | | | | 7.24 (m, 4H), 7.21-7.17 (m, 1H), 6.94 |
| | | | | (d, J = 9.2 Hz, 2H), 6.90-6.83 (m, 1H), |
| | | | | 6.72 (d, J = 8.4 Hz, 2H), 5.37 (d, J = 4.4 |
| | | | | Hz, 1H), 5.14-5.08 (m, 1H), 4.97-4.87 |
| | | | | (m, 1H), 4.52 (d, J = 9.2 Hz, 1H), 4.43 (t, |
| | | | | J = 8.0 Hz, 1H), 4.40-4.35 (m, 1H), 4.28 |
| | | | | (s, 1H), 4.06-3.96 (m, 1H), 3.71-3.67 |
| | | | | (m, 2H), 3.63-3.50 (m, 6H), 2.63 ( d, J = |
| | | | | 11.2 Hz, 3H), 2.57 (d, J = 2.0 Hz, 2H), |
| | | | | 2.46 (s, 6H), 2.07-1.91 (m, 5H), 1.86- |
| | | | | 1.76 (m, 3H), 1.74-1.57 (m, 6H), 1.49- |
| | | | | 1.43 (m, 1H), 1.38 (d, J = 6.8 Hz, 3H), |
| | | | | 1.23 (s, 1H), 1.18-1.11 (m, 1H), 1.08- |
| | | | | 0.99 (m, 2H), 0.97-0.93 (m, 9H), 0.87- |
| | | | | 0.83 (m, 1H) |
| I-29 | KS | KQ | 1452.4 | 8.34 (d, J = 8.0 Hz, 1H), 8.15 (s, 1H), |
| | | | | 7.97 ( d, J = 9.0 Hz, 1H), 7.77 (d, J = 9.4 |
| | | | | Hz, 1H), 7.67 (d, J = 9.0 Hz, 2H), 7.59- |
| | | | | 7.56 (m, 1H), 7.49-7.46 (m, 2H), 7.43- |
| | | | | 7.40 (m, 3H), 7.37-7.22 (m, 13H), 7.19- |
| | | | | 7.10 (m, 3H), 7.10-7.06 (m, 1H), 7.00- |
| | | | | 6.94 (m, 1H), 6.83-6.80 (m, 2H), 5.22 |
| | | | | (d, J = 4.0 Hz, 1H), 5.08 (d, J = 3.4 Hz, |
| | | | | 1H), 4.87 (t, J = 7.4 Hz, 1H), 4.50 (d, J = |
| | | | | 9.5 Hz, 1H), 4.39 (t, J = 7.8 Hz, 1H), |
| | | | | 4.31-4.25 (m, 2H), 4.12 (s, 1H), 3.88-3.83 |
| | | | | (m, 1H), 3.72-3.67 (m, 1H), 3.59 (d, J = |
| | | | | 4.0 Hz, 2H), 3.39 (br d, J = 2.8 Hz, 1H), |
| | | | | 2.72-2.68 (m, 1H), 2.61-2.54 (m, 2H), |
| | | | | 2.28-2.19 (m, 5H), 2.14-2.04 (m, 3H), |
| | | | | 2.01-1.96 (m, 2H), 1.92-1.85 (m, 2H), |
| | | | | 1.75 (ddd, J = 4.6, 8.6, 12.8 Hz, 2H), |
| | | | | 1.68-1.64 (m, 1H), 1.51-1.40 (m, 6H), 1.33 |
| | | | | (d, J = 7.0 Hz, 3H), 1.10-1.05 (m, 1H), |
| | | | | 1.04-0.97 (m, 2H), 0.92 (s, 9H), 0.91 (s, |
| | | | | 1H), 0.87-0.75 (m, 2H), 0.36-0.34 (m, |
| | | | | 1H |
| I-30 | KZ | KT | 1612.6 | 8.98 (s, 1H), 8.36 (d, J = 7.6 Hz, 1H), |
| | | | | 8.08 (s, 1H), 7.95 (d, J = 7.6 Hz, 1H), |
| | | | | 7.78 (d, J = 9.2 Hz, 1H), 7.66 (d, J = 8.8 |
| | | | | Hz, 2H), 7.53 (s, 1H), 7.48 (d, J = 8.4 Hz, |
| | | | | 2H), 7.44-7.42 (m, 2H), 7.38 (s, 1H), |
| | | | | 7.37-7.35 (m, 2H), 7.33 (s, 2H), 7.30 (d, |
| | | | | J = 7.6 Hz, 2H), 7.27-7.21 (m, 2H), 7.20- |
| | | | | 7.17 (m, 1H), 7.10 (d, J = 7.6 Hz, 1H), |
| | | | | 6.89 (d, J = 7.6 Hz, 1H), 6.72 (d, J = 8.4 |
| | | | | Hz, 3H), 5.22 (d, J = 4.4 Hz, 1H), 5.09 |
| | | | | (d, J = 3.2 Hz, 1H), 4.91 (t, J = 7.6 Hz, |
| | | | | 1H), 4.51 (d, J = 9.6 Hz, 1H), 4.42 ( t, |
| | | | | J = 8.0 Hz, 1H), 4.34-4.26 (m, 2H), 4.05- |
| | | | | 3.98 (m, IH), 3.76-3.67 (m, 2H), 3.60 |
| | | | | (s, 2H), 3.55 (s, 2H), 3.45 (s, 6H), 3.27- |
| | | | | 3.25 (m, 1H), 2.97-2.89 (m, 2H), 2.45 |
| | | | | (s, 3H), 2.40 (s, 4H), 2.34 (d, J = 5.6 Hz, |
| | | | | 4H), 2.30-2.17 (m, 5H), 2.12 (dd, J = |
| | | | | 7.2, 13.8 Hz, 2H), 2.04-1.98 (m, 2H), |
| | | | | 1.96-1.85 (m, 2H), 1.85-1.68 (m, 2H), |
| | | | | 1.61-1.56 (m, 1H), 1.50-1.45 (m, 4H), |
| | | | | 1.37 (d, J = 7.2 Hz, 3H), 1.29-1.22 (m, |
| | | | | 3H), 1.12 (d, J = 10.0 Hz, 2H), 1.03- |
| | | | | 0.97 (m, 2H), 0.93 (s, 9H) |
| I-33 | KP | KQ | 1451.2 | 8.37-8.31 (m, 1H), 8.13 (s, 1H), 7.95 |
| | | | | (dd, J = 1.6, 9.2 Hz, 1H), 7.77 (d, J = 9.4 |
| | | | | Hz, 1H), 7.67 (d, J = 9.0 Hz, 2H), 7.58 |
| | | | | (d, J = 7.2 Hz, 1H), 7.48 (d, J = 8.4 Hz, |
| | | | | 2H), 7.45-7.38 (m, 4H), 7.36-7.30 (m, |
| | | | | 6H), 7.30-7.23 (m, 5H), 7.22-7.11 (m, |
| | | | | 3H), 6.94 (s, 3H), 6.81-6.77 (m, 2H), |
| | | | | 5.22 (d, J = 4.4 Hz, 1H), 5.08 (d, J = 3.6 |
| | | | | Hz, 1H), 4.87 (t, J = 7.4 Hz, 1H), 4.50 (d, |
| | | | | J = 9.4 Hz, 1H), 4.39 (t, J = 8.0 Hz, 1H), |
| | | | | 4.32-4.25 (m, 2H), 4.13-4.05 (m, 2H), |
| | | | | 3.85-3.80 (m, 1H), 3.73-3.62 (m, 2H), |
| | | | | 3.56 (s, 3H), 2.69-2.66 (m, 1H), 2.40- |
| | | | | 2.36 (m, 1H), 2.25-2.19 (m, 4H), 2.10 |
| | | | | (dd, J = 6.4, 14.2 Hz, 2H), 2.01-1.95 (m, |
| | | | | 2H), 1.88 (d, J = 10.8 Hz, 1H), 1.71 ( s, |
| | | | | 2H), 1.70-1.53 (m, 2H), 1.49-1.40 (m, |
| | | | | 5H), 1.34-1.31 (m, 3H), 1.27-1.17 (m, |
| | | | | 3H), 1.11-1.06 (m, 1H), 1.03-0.96 (m, |
| | | | | 2H), 0.94-0.88 (m, 10H), 0.85-0.78 (m, |
| | | | | 1H) |
| I-35 | LZ | KT | 1422.5 | 8.98 (s, 1H), 8.49 (d, J = 2.8 Hz, 1H), |
| | | | | 8.37 (d, J = 8.0 Hz, 1H), 7.83-7.77 (m, |
| | | | | 2H), 7.72 (d, J = 8.8 Hz, 2H), 7.52 (s, |
| | | | | 1H), 7.47 (s, 4H), 7.44 (s, 1H), 7.42 (s, |
| | | | | 1H), 7.39-7.35 (m, 4H), 7.34-7.27 (m, |
| | | | | 3H), 7.26-7.18 (m, 4H), 7.15 (d, J = 6.8 |
| | | | | Hz, 1H), 6.88-6.81 (m, 2H), 5.09 (d, J = |
| | | | | 3.6 Hz, 1H), 4.90 (d, J = 6.4 Hz, 1H), |
| | | | | 4.50 (d, J = 9.2 Hz, 1H), 4.41 (t, J = 8.0 |
| | | | | Hz, 1H), 4.31-4.24 (m, 1H), 4.19-4.07 |
| | | | | (m, 1H), 3.59 (s, 2H), 3.38 (d, J = 1.6 Hz, |
| | | | | 2H), 3.24-3.14 (m, 6H), 2.39 (d, J = 5.6 |
| | | | | Hz, 3H), 2.35-2.30 (m, 8H), 2.24-2.19 |
| | | | | (m, 4H), 2.13-2.08 (m, 2H), 2.04-1.97 |
| | | | | (m, 3H), 1.87-1.74 (m, 3H), 1.53-1.41 |
| | | | | (m, 6H), 1.37 (d, J = 7.2 Hz, 3H), |
| | | | | 1.27-1.19 (m, 3H), 0.92 (s, 9H) |
| I-38 | MJ | KT | 1534.4 | 11.66 (s, 1H), 8.98 (s, 1H), 8.36 (d, J = |
| | | | | 7.6 Hz, 1H), 8.11 (s, 1H), 8.00 (s, 1H), |
| | | | | 7.94-7.83 (m, 1H), 7.79 (d, J = 9.2 Hz, |
| | | | | 1H), 7.53 (d, J = 7.6 Hz, 1H), 7.50-7.46 |
| | | | | (m, 3H), 7.46-7.43 (m, 3H), 7.43-7.40 |
| | | | | (m, 2H), 7.39-7.36 (m, 3H), 7.32-7.26 |
| | | | | (m, 4H), 7.10 (d, J = 7.6 Hz, 1H), 7.02- |
| | | | | 6.93 (m, 1H), 6.60 (d, J = 7.6 Hz, 1H), |
| | | | | 6.38 (s, 1H), 6.12 (s, 1H), 5.19 (d, J = 4.4 |
| | | | | Hz, 1H), 5.09 (d, J = 3.6 Hz, 1H), 4.96- |
| | | | | 4.88 (m, 1H), 4.53-4.48 (m, 1H), 4.42 |
| | | | | (t, J = 8.0 Hz, 1H), 4.25 (dt, J = 4.4, 7.6 |
| | | | | Hz, 2H), 3.60 (s, 2H), 3.43 (s, 3H), 3.36 |
| | | | | (d, J = 5.6 Hz, 2H), 3.30 (s, 2H), 2.56 (s, |
| | | | | 2H), 2.45 (s, 3H), 2.38 (d, J = 2.0 Hz, |
| | | | | 4H), 2.25 (t, J = 7.6 Hz, 4H), 2.14-2.09 |
| | | | | (m, 1H), 2.03-1.97 (m, 1H), 1.83-1.70 |
| | | | | (m, 5H), 1.51-1.43 (m, 5H), 1.37 (d, J = |
| | | | | 7.2 Hz, 3H), 1.24 (q, J = 7.2 Hz, 3H), |
| | | | | 1.03-0.97 (m, 1H), 0.93 (s, 12H), 0.78- |
| | | | | 0.69 (m, 1H) |
| I-42 | OM | KT | 1541.6 | 8.99 (s, 1H), 8.37 (d, J = 7.6 Hz, 1H), |
| | | | | 8.09 (d, J = 1.6 Hz, 1H), 7.93 (dd, J = |
| | | | | 9.2, 1.6 Hz, 1H), 7.79 (d, J = 9.2 Hz, 1H), |
| | | | | 7.72 (d, J = 8.8 Hz, 2H), 7.42-7.46 (m, |
| | | | | 2H), 7.36-7.40 (m, 4H), 7.35 (s, 2H), |
| | | | | 7.29 (t, J = 7.6 Hz, 2H), 7.16-7.23 (m, |
| | | | | 1H), 7.09 (d, J = 8.4 Hz, 2H), 6.87-6.93 |
| | | | | (m, 1H), 6.76-6.83 (m, 3H), 5.10 (d, J = |
| | | | | 3.6 Hz, 1H), 4.88-4.95 (m, 1H), 4.52 |
| | | | | (d, J = 9.2 Hz, 1H), 4.43 (t, J = 8.0 Hz, |
| | | | | 1H), 4.28 (br d, J = 2.0 Hz, 1H), 3.98- |
| | | | | 4.08 (m, 1H), 3.57-3.64 (m, 2H), 3.37- |
| | | | | 3.48 (m, 2H), 3.22-3.29 (m, 2H), 3.17 |
| | | | | (br s, 4H), 2.82 (br s, 2H), 2.46 (s, 3H), |
| | | | | 2.30 (br s, 9H), 2.18-2.26 (m, 7H), 2.07- |
| | | | | 2.16 (m, 2H), 1.99 (br s, 4H), 1.71- |
| | | | | 1.83 (m, 2H), 1.40-1.53 (m, 7H), 1.38 |
| | | | | (d, J = 6.8 Hz, 3H), 1.20-1.27 (m, 2H), |
| | | | | 0.96 (s, 6H), 0.94 (s, 9H) |
| I-51 | NR | KT | 1519.5 | 11.66 (s, 1H), 9.01-8.95 (m, 1H), 8.32 |
| | | | | (s, 1H), 8.16-8.08 (m, 1H), 8.06-7.96 |
| | | | | (m, 1H), 7.93-7.84 (m, 1H), 7.82-7.75 |
| | | | | (m, 1H), 7.52-7.48 (m, 2H), 7.47-7.41 |
| | | | | (m, 8H), 7.31 (s, 5H), 7.22 (dd, J = 2.0, |
| | | | | 6.8 Hz, 1H), 7.06-6.88 (m, 1H), 6.73- |
| | | | | 6.65 (m, 1H), 6.40-6.35 (m, 1H), 6.25- |
| | | | | 6.18 (m, 1H), 5.15-5.06 (m, 1H), 4.97- |
| | | | | 4.87 (m, 1H), 4.54-4.48 (m, 1H), 4.45- |
| | | | | 4.38 (m, 1H), 4.30-4.24 (m, 1H), 3.63- |
| | | | | 3.58 (m, 2H), 3.49-3.40 (m, 8H), 3.09- |
| | | | | 3.03 (m, 4H), 2.58 (d, J = 2.0 Hz, 4H), |
| | | | | 2.45 (s, 3H), 2.34-2.28 (m, 6H), 2.28- |
| | | | | 2.19 (m, 4H), 2.11 (dd, J = 6.4, 13.6 Hz, |
| | | | | 1H), 2.03-1.97 (m, 1H), 1.83-1.77 (m, |
| | | | | 1H), 1.76-1.69 (m, 2H), 1.54-1.41 (m, |
| | | | | 5H), 1.37 (d, J = 6.8 Hz, 3H), 1.27-1.21 |
| | | | | (m, 2H), 0.93 (s, 9H) |
| I-52 | QH | KT | 1551.6 | 11.64 (s, 1H), 9.02-8.94 (m, 1H), 8.40- |
| | | | | 8.33 (m, 1H), 8.19-8.07 (m, 1H), 8.06- |
| | | | | 7.98 (m, 1H), 7.95-7.86 (m, 1H), 7.83- |
| | | | | 7.75 (m, 1H), 7.53-7.46 (m, 3H), 7.45- |
| | | | | 7.39 (m, 3H), 7.38-7.33 (m, 4H), 7.07 |
| | | | | (d, J = 8.4 Hz, 2H), 7.01 (dd, J = 2.8, 10.0 |
| | | | | Hz, 1H), 6.69-6.64 (m, 1H), 6.42-6.37 |
| | | | | (m, 1H), 6.18 (s, 1H), 5.09 (d, J = 3.6 Hz, |
| | | | | 1H), 4.97 (s, 1H), 4.55-4.48 (m, 1H), |
| | | | | 4.46-4.39 (m, 1H), 4.31-4.24 (m, 1H), |
| | | | | 3.63-3.57 (m, 2H), 3.48-3.40 (m, 8H), |
| | | | | 3.07-3.01 (m, 4H), 2.61 (s, 3H), 2.45 (s, |
| | | | | 3H), 2.29-2.23 (m, 4H), 2.18 (s, 6H), |
| | | | | 2.14-2.06 (m, 2H), 2.05-1.96 (m, 2H), |
| | | | | 1.93 (s, 2H), 1.83-1.78 (m, 1H), 1.75- |
| | | | | 1.69 (m, 2H), 1.50-1.44 (m, 4H), 1.40- |
| | | | | 1.34 (m, 5H), 1.31-1.19 (m, 4H), 0.93 |
| | | | | (s, 15H) |
| I-55 | OD | KT | 1465.5 | 11.78 (s, 1 H), 11.47 (s, 1 H), 9.02 (s, 1 |
| | | | | H), 8.38 (d, J = 8.0 Hz, 1 H), 8.18 (d, J = |
| | | | | 2.0 Hz, 1 H), 8.06 (d, J = 2.4 Hz, 1 H), |
| | | | | 7.94 (J = 9.2, 2.0 Hz, 1 H), 7.86 (t, J = |
| | | | | 5.6 Hz, 1 H), 7.79 (d, J = 9.2 Hz, 1 H), |
| | | | | 7.60 (d, J = 2.4 Hz, 1 H), 7.51-7.55 (m, |
| | | | | 2 H), 7.44-7.49 (m, 4 H), 7.43 (s, 2 H), |
| | | | | 7.36-7.41 (m, 3 H), 7.28-7.33 (m, 3 H), |
| | | | | 7.09-7.13 (m, 1 H), 7.06 (d, J = 9.6 Hz, |
| | | | | 1 H), 6.66 (d, J = 8.4 Hz, 1 H), 6.43 (J = |
| | | | | 3.2, 2.0 Hz, 1 H), 6.14 (s, 1 H), 4.92 (t, |
| | | | | J = 7.2 Hz, 1 H), 4.51 (d, J = 9.2 Hz, 1 H), |
| | | | | 4.42 (t, J = 8.0 Hz, 1 H), 4.28 (s, 1 H), |
| | | | | 4.24 (d, J = 7.6 Hz, 1 H), 3.60 (s, 3 H), |
| | | | | 3.41-3.46 (m, 1 H), 3.36 (d, J = 6.4 Hz, |
| | | | | 2 H), 3.08 (d, J = 6.0 Hz, 2 H), 2.62 (s, 1 |
| | | | | H), 2.46 (s, 3 H), 2.23 (dd, J = 14.4, 7.2 |
| | | | | Hz, 1 H), 2.09-2.15 (m, 1 H), 2.00-2.08 |
| | | | | (m, 3 H), 1.75-1.84 (m, 2 H), 1.63 (t, J = |
| | | | | 6.4 Hz, 3 H), 1.40-1.57 (m, 5 H), 1.37 |
| | | | | (d, J = 7.2 Hz, 3 H), 1.19-1.26 (m, 2 H), |
| | | | | 0.97-1.06 (m, 1 H), 0.93 (s, 9 H), 0.68- |
| | | | | 0.78 (m, 1 H) |
| I-56 | OE | KT | 1465.5 | 11.76 (s, 1H), 11.45 (s, 1H), 9.00 (s, 1H), |
| | | | | 8.37 (d, J = 7.8 Hz, 1H), 8.17 (d, J = 2.0 |
| | | | | Hz, 1H), 8.05 (d, J = 2.4 Hz, 1H), 7.93 |
| | | | | (dd, J = 2.4, 9.2 Hz, 1H), 7.85 (t, J = 5.8 |
| | | | | Hz, 1H), 7.78 (d, J = 9.2 Hz, 1H), 7.58 |
| | | | | (d, J = 2.4 Hz, 1H), 7.53 (dd, J = 2.4, 5.6 |
| | | | | Hz, 2H), 7.47 (s, 1H), 7.46-7.43 (m, |
| | | | | 3H), 7.42 (s, 2H), 7.39-7.36 (m, 3H), |
| | | | | 7.31 (s, 1H), 7.30-7.26 (m, 2H), 7.10 |
| | | | | (dd, J = 1.2, 7.6 Hz, 1H), 7.05 (d, J = 9.6 |
| | | | | Hz, 1H), 6.65 (d, J = 8.8 Hz, 1H), 6.42 |
| | | | | (dd, J = 2.0, 3.6 Hz, 1H), 6.12 (s, 1H), |
| | | | | 4.94-4.88 (m, 1H), 4.51 (d, J = 9.2 Hz, |
| | | | | 1H), 4.42 (t, J = 8.0 Hz, 1H), 4.27 (s, |
| | | | | 1H), 4.23 (d, J = 8.0 Hz, IH), 3.45 (s, |
| | | | | 1H), 3.41 (d, J = 0.8 Hz, 1H), 3.38-3.32 |
| | | | | (m, 3H), 3.07 (q, J = 6.4 Hz, 2H), 2.59 |
| | | | | (d, J = 13.2 Hz, 2H), 2.45 (s, 3H), 2.30- |
| | | | | 2.17 (m, 2H), 2.15-2.10 (m, 1H), 2.09- |
| | | | | 2.02 (m, 3H), 1.99 (d, J = 8.4 Hz, 1H), |
| | | | | 1.82-1.75 (m, 2H), 1.65-1.58 (m, 3H), |
| | | | | 1.53-1.43 (m, 5H), 1.37 (d, J = 7.2 Hz, |
| | | | | 3H), 1.26-1.19 (m, 2H), 0.92 (s, 9H), |
| | | | | 0.77-0.69 (m, 1H) |
| I-57 | OH | KT | 1334.3 | 11.61 (s, 1H), 8.99 (s, 1H), 8.38 (d, J = |
| | | | | 7.6 Hz, 1H), 8.06 (s, 1H), 7.99 (d, J = 2.4 |
| | | | | Hz, 1H), 7.80 (d, J = 9.6 Hz, 2H), 7.56 |
| | | | | (d, J = 8.8 Hz, 1H), 7.49-7.30 (m, 7H), |
| | | | | 7.16 (s, 1H), 6.86 (d, J = 8.8 Hz, 1H), |
| | | | | 6.72 (d, J = 9.2 Hz, 1H), 6.36 (s, 1H), |
| | | | | 6.32 (s, 1H), 5.10 (s, 1H), 4.92 (t, J = 7.2 |
| | | | | Hz, 1H), 4.52 (d, J = 9.2 Hz, 1H), 4.43 (t, |
| | | | | J = 7.6 Hz, 1H), 4.28 (s, 1H), 3.61 (s, |
| | | | | 2H), 3.46-3.42 (m, 4H), 3.21-3.15 (m, |
| | | | | 4H), 2.68 (s, 1H), 2.62 (s, 4H), 2.47- |
| | | | | 2.45 (m, 3H), 2.36 (s, 8H), 2.26 (t, J = 7.6 |
| | | | | Hz, 3H), 2.23-2.19 (m, 1H), 2.16-1.97 |
| | | | | (m, 3H), 1.84-1.70 (m, 3H), 1.52-1.44 |
| | | | | (m, 4H), 1.38 (d, J = 7.2 Hz, 3H), 1.28- |
| | | | | 1.21 (m, 2H), 0.94 (s, 9H) |
| I-58 | OA | KT | 1479.5 | 11.77-11.60 (m, 1H), 11.41 (s, 1H), 8.99 |
| | | | | (s, 1H), 8.37 (d, J = 8.0 Hz, 1H), 8.25- |
| | | | | 8.08 (m, 1H), 8.07-7.98 (m, 1H), 7.97- |
| | | | | 7.84 (m, 1H), 7.83-7.74 (m, 1H), 7.53 |
| | | | | (d, J = 8.0 Hz, 2H), 7.49-7.44 (m, 4H), |
| | | | | 7.43-7.41 (m, 2H), 7.40-7.36 (m, 3H), |
| | | | | 7.34-7.26 (m, 4H), 7.11 (dd, J = 1.2, 7.6 |
| | | | | Hz, 1H), 6.62 d, J = 8.4 Hz, 1H), 6.45- |
| | | | | 6.36 (m, 1H), 6.17-6.05 (m, 1H), 5.19 |
| | | | | (d, J = 4.4 Hz, 1H), 5.10 (d, J = 3.6 Hz, |
| | | | | 1H), 4.97-4.88 (m, 1H), 4.54-4.49 (m, |
| | | | | 1H), 4.42 (t, J = 8.0 Hz, 1H), 4.29-4.22 |
| | | | | (m, 2H), 3.65-3.57 (m, 1H), 3.60 (s, |
| | | | | 3H), 3.47-3.39 (m, 1H), 3.30-3.26 (m, |
| | | | | 2H), 2.96-2.92 (m, 2H), 2.78 (s, 1H), |
| | | | | 2.63-2.53 (m, 1H), 2.46 (s, 4H), 2.25 |
| | | | | (td, J = 7.6, 16.0 Hz, 4H), 2.16-2.09 (m, |
| | | | | 1H), 2.04-1.98 (m, 1H), 1.85-1.74 (m, |
| | | | | 3H), 1.72-1.55 (m, 3H), 1.54-1.42 (m, |
| | | | | 5H), 1.37 (d, J = 7.2 Hz, 3H), 1.29-1.19 |
| | | | | (m, 3H), 1.05-0.97 (m, 1H), 0.93 (s, |
| | | | | 9H), 0.79-0.68 (m, 1H) |
| I-59 | MH | OF | 1506.5 | 11.66 (s, 1H), 9.05-8.86 (m, 1H), 8.37 |
| | | | | (d, J = 8.0 Hz, 1H), 8.12 (d, J = 11.2 Hz, |
| | | | | 1H), 8.00 (d, J = 2.0 Hz, 1H), 7.91-7.80 |
| | | | | (m, 2H), 7.53 (d, J = 7.8 Hz, 1H), 7.49- |
| | | | | 7.44 (m, 5H), 7.40 (d, J = 14.0 Hz, 4H), |
| | | | | 7.36 (d, J = 1.6 Hz, 1H), 7.32-7.26 (m, |
| | | | | 4H), 7.10 (dd, J = 1.2, 7.6 Hz, 1H), 7.01- |
| | | | | 6.91 (m, 1H), 6.64-6.57 (m, 1H), 6.37 |
| | | | | (s, 1H), 6.12 (s, 1H), 5.20 (d, J = 4.4 Hz, |
| | | | | 1H), 5.10 (d, J = 3.2 Hz, 1H), 4.91 (quin, |
| | | | | J = 7.2 Hz, 1H), 4.51 (d, J = 9.2 Hz, 1H), |
| | | | | 4.42 (t, J = 8.0 Hz, 1H), 4.29-4.21 (m, |
| | | | | 2H), 3.63-3.56 (m, 3H), 3.42 (s, 6H), |
| | | | | 3.26-3.22 (m, 2H), 2.60-2.53 (m, 1H), |
| | | | | 2.45 (s, 3H), 2.38 (s, 4H), 2.33 (s, 2H), |
| | | | | 2.30-2.22 (m, 4H), 2.22-2.12 (m, 2H), |
| | | | | 2.04-1.98 (m, 1H), 1.79 (dt, J = 4.4, 8.4 |
| | | | | Hz, 2H), 1.74-1.65 (m, 4H), 1.61-1.54 |
| | | | | (m, 1H), 1.37 (d, J = 7.2 Hz, 3H), 1.06- |
| | | | | 0.98 (m, 1H), 0.95-0.91 (m, 9H), 0.76- |
| | | | | 0.67 (m, 1H) |
| I-60 | MJ | OF | 1506.5 | 8.98 (s, 1H), 8.50 (t, J = 5.6 Hz, 1H), 8.13 |
| | | | | (s, 1H), 8.05 (d, J = 1.2 Hz, 1H), 7.90 (d, |
| | | | | J = 9.2 Hz, 1H), 7.68 (d, J = 8.8 Hz, 2H), |
| | | | | 7.55 (d, J = 7.6 Hz, 1H), 7.41 (d, J = 7.6 |
| | | | | Hz, 1H), 7.35-7.32 (m, 2H), 7.30-7.25 |
| | | | | (m, 4H), 7.18-7.14 (m, 2H), 7.02 (s, |
| | | | | 1H), 6.96 (d, J = 7.6 Hz, 2H), 6.85 (d, |
| | | | | J = 8.8 Hz, 1H), 6.75 (d, J = 8.8 Hz, 2H), |
| | | | | 6.71-6.67 (m, 1H), 5.17 (d, J = 3.6 Hz, |
| | | | | 1H), 4.59 (d, J = 9.2 Hz, 1H), 4.52 (t, J = |
| | | | | 8.4 Hz, 1H), 4.34 (d, J = 4.8 Hz, 2H), |
| | | | | 4.28 (d, J = 6.4 Hz, 1H), 4.22 (d, J = 4.8 |
| | | | | Hz, 1H), 4.18-4.14 (m, 2H), 4.02-3.97 |
| | | | | (m, 1H), 3.68-3.57 (m, 4H), 3.13-3.06 |
| | | | | (m, 4H), 3.00 (s, 4H), 2.84-2.75 (m, |
| | | | | 2H), 2.54 (s, 2H), 2.45 (s, 3H), 2.12- |
| | | | | 2.03 (m, 4H), 1.95-1.88 (m, 4H), 1.76- |
| | | | | 1.68 (m, 2H), 1.63-1.56 (m, 2H), 1.52- |
| | | | | 1.31 (m, 12H), 1.26-1.18 (m, 4H), 0.94 |
| | | | | (s, 9H), 0.69-0.55 (m, 3H) |
| I-61 | OZ | KT | 1464.5 | 11.68 (s, 1H), 11.57-11.45 (m, 1H), 9.05- |
| | | | | 8.90 (m, 1H), 8.42-8.31 (m, 1H), 8.22- |
| | | | | 8.10 (m, 1H), 8.08-8.01 (m, 1H), 7.96- |
| | | | | 7.86 (m, 1H), 7.83-7.74 (m, 1H), 7.48 |
| | | | | (s, 3H), 7.47-7.41 (m, 8H), 7.31 (d, J = |
| | | | | 2.0 Hz, 5H), 7.23-7.20 (m, 1H), 7.07- |
| | | | | 6.94 (m, 1H), 6.69 (d, J = 8.6 Hz, 1H), |
| | | | | 6.44-6.33 (m, 1H), 6.24-6.18 (m, 1H), |
| | | | | 5.14-5.05 (m, 1H), 4.95-4.87 (m, 1H), |
| | | | | 4.51 (d, J = 9.6 Hz, 1H), 4.45-4.38 (m, |
| | | | | 1H), 4.31-4.24 (m, 1H), 3.65-3.54 (m, |
| | | | | 2H), 3.30-3.26 (m, 3H), 3.07 (s, 4H), |
| | | | | 2.94 (s, 2H), 2.80-2.76 (m, 1H), 2.45 (s, |
| | | | | 3H), 2.30 (s, 5H), 2.27-2.19 (m, 3H), |
| | | | | 2.17-2.03 (m, 2H), 2.03-1.92 (m, 1H), |
| | | | | 1.84-1.71 (m, 2H), 1.67 ( t, J = 6.4 Hz, |
| | | | | 1H), 1.57-1.40 (m, 5H), 1.36 (d, J = 7.2 |
| | | | | Hz, 3H), 1.29-1.21 (m, 2H), 0.92 (s, 9H) |
| I-62 | PA | KT | 1450.4 | 11.75-11.64 (m, 1H), 11.58-11.41 (m, |
| | | | | 1H), 8.98 (s, 1H), 8.39-8.34 (m, 1H), |
| | | | | 8.21-8.11 (m, 1H), 8.08-8.01 (m, 1H), |
| | | | | 7.97-7.86 (m, 1H), 7.84 (t, J = 5.6 Hz, |
| | | | | 1H), 7.80-7.76 (m, 1H), 7.56-7.48 (m, |
| | | | | 3H), 7.47-7.41 (m, 8H), 7.39-7.32 (m, |
| | | | | 5H), 7.23-7.20 (m, 1H), 7.06-6.95 (m, |
| | | | | 1H), 6.69 (d, J = 8.8 Hz, 1H), 6.42-6.36 |
| | | | | (m, 1H), 6.21 (s, 1H), 5.10 (d, J = 3.6 Hz, |
| | | | | 1H), 4.94-4.87 (m, 1H), 4.51 (d, J = 9.6 |
| | | | | Hz, 1H), 4.42 (t, J = 8.0 Hz, 1H), 4.27 (d, |
| | | | | J = 2.8 Hz, 1H), 3.64-3.55 (m, 2H), 3.07 |
| | | | | (d, J = 5.5 Hz, 7H), 2.45 (s, 3H), 2.33- |
| | | | | 2.26 (m, 5H), 2.25 (s, 1H), 2.15-2.02 |
| | | | | (m, 4H), 1.99 (dd, J = 2.4, 8.3 Hz, 1H), |
| | | | | 1.82-1.76 (m, 1H), 1.62 (t, J = 6.4 Hz, |
| | | | | 2H), 1.55-1.42 (m, 5H), 1.37 (d, J = 7.2 |
| | | | | Hz, 3H), 1.25-1.19 (m, 2H), 0.92 (s, 9H) |
| I-63 | LB | MK | 1537.6 | 11.71-11.64 (m, 1H), 9.02-8.96 (m, |
| | | | | 1H), 8.41-8.34 (m, 1H), 8.17-8.10 (m, |
| | | | | 1H), 8.03 (d, J = 1.6 Hz, 1H), 7.95-7.87 |
| | | | | (m, 1H), 7.84-7.77 (m, 1H), 7.53-7.47 |
| | | | | (m, 3H), 7.45-7.42 (m, 2H), 7.36 (J = |
| | | | | 8.4, 13.8 Hz, 5H), 7.07-6.99 (m, 3H), |
| | | | | 6.70-6.65 (m, 1H), 6.42-6.36 (m, 1H), |
| | | | | 6.20 (s, 1H), 5.17-5.06 (m, 1H), 4.96- |
| | | | | 4.89 (m, 1H), 4.52 (d, J = 9.6 Hz, 1H), |
| | | | | 4.43 (t, J = 8.0 Hz, 1H), 4.31-4.25 (m, |
| | | | | 1H), 3.65-3.58 (m, 2H), 3.48-3.42 (m, |
| | | | | 4H), 3.06 (s, 4H), 2.74 (s, 2H), 2.46 (s, |
| | | | | 3H), 2.33 (s, 2H), 2.29 (s, 3H), 2.19 (s, |
| | | | | 4H), 2.15 (s, 4H), 2.11-2.04 (m, 2H), |
| | | | | 2.03-1.97 (m, 2H), 1.96 (s, 2H), 1.84- |
| | | | | 1.69 (m, 4H), 1.51-1.44 (m, 4H), 1.40- |
| | | | | 1.35 (m, 5H), 1.24 (s, 1H), 1.12-1.02 |
| | | | | (m, 1H), 0.93 (d, J = 4.0 Hz, 15H) |
| I-64 | KZ | LG | 1626.8 | 8.98 (s, 1H), 8.50 (t, J = 5.6 Hz, 1H), 8.13 |
| | | | | (s, 1H), 8.05 (d, J = 1.2 Hz, IH), 7.90 (d, |
| | | | | J = 9.2 Hz, 1H), 7.68 (d, J = 8.8 Hz, 2H), |
| | | | | 7.55 (d, J = 7.6 Hz, 1H), 7.41 (d, J = 7.6 |
| | | | | Hz, 1H), 7.35-7.32 (m, 2H), 7.30-7.25 |
| | | | | (m, 4H), 7.18-7.14 (m, 2H), 7.02 (s, |
| | | | | 1H), 6.96 (d, J = 7.6 Hz, 2H), 6.85 (d, |
| | | | | J = 8.8 Hz, 1H), 6.75 (d, J = 8.8 Hz, 2H), |
| | | | | 6.71-6.67 (m, 1H), 5.17 (d, J = 3.6 Hz, |
| | | | | 1H), 4.59 (d, J = 9.2 Hz, 1H), 4.52 (t, J = |
| | | | | 8.4 Hz, 1H), 4.34 (d, J = 4.8 Hz, 2H), |
| | | | | 4.28 (d, J = 6.4 Hz, 1H), 4.22 (d, J = 4.8 |
| | | | | Hz, 1H), 4.18-4.14 (m, 2H), 4.02-3.97 |
| | | | | (m, 1H), 3.68-3.57 (m, 4H), 3.13-3.06 |
| | | | | (m, 4H), 3.00 (s, 4H), 2.84-2.75 (m, |
| | | | | 2H), 2.54 (s, 2H), 2.45 (s, 3H), 2.12- |
| | | | | 2.03 (m, 4H), 1.95-1.88 (m, 4H), 1.76- |
| | | | | 1.68 (m, 2H), 1.63-1.56 (m, 2H), 1.52- |
| | | | | 1.31 (m, 12H), 1.26-1.18 (m, 4H), 0.94 |
| | | | | (s, 9H), 0.69-0.55 (m, 3H) |
| I-65 | NV | LG | 1650.7 | 11.65 (br s, 1H), 8.99 (s, 1H), 8.37 (d, |
| | | | | J = 8.0 Hz, 1H), 8.10 (s, 1H), 8.01 (d, J = |
| | | | | 2.4 Hz, 1H), 7.86 (br d, J = 8.8 Hz, 1H), |
| | | | | 7.79 (br d, J = 9.2 Hz, 1H), 7.48-7.53 |
| | | | | (m, 2H), 7.41-7.47 (m, 3H), 7.33-7.40 |
| | | | | (m, 4H), 7.23-7.31 (m, 1H), 7.09 (br d, |
| | | | | J = 8.4 Hz, 2H), 6.90-7.01 (m, 1H), 6.66 |
| | | | | (br d, J = 8.0 Hz, 1H), 6.38 (br s, 1H), |
| | | | | 6.20 (s, 1H), 5.10 (d, J = 3.6 Hz, 1H), |
| | | | | 4.89-4.96 (m, 1H), 4.52 (d, J = 9.2 Hz, |
| | | | | 1H), 4.43 (t, J = 8.0 Hz, 1H), 4.28 (br s, |
| | | | | 1H), 3.55-3.64 (m, 6H), 3.36-3.42 (m, |
| | | | | 5H), 3.04 (br s, 4H), 2.66-2.72 (m, 2H), |
| | | | | 2.46 (s, 3H), 2.36-2.44 (m, 9H), 2.22- |
| | | | | 2.27 (m, 4H), 2.18 (br s, 6H), 2.06-2.15 |
| | | | | (m, 3H), 1.98-2.06 (m, 2H), 1.65-1.93 |
| | | | | (m, 5H), 1.41-1.55 (m, 6H), 1.38 (br d, |
| | | | | J = 6.8 Hz, 3H), 1.24 (br s, 5H), 0.92- |
| | | | | 0.97 (m, 9H), 0.90 (s, 3H) |
| I-70 | OV | KT | 1455.5 | 12.16-11.85 (m, 1H), 8.99 (s, 1H), 8.37 |
| | | | | (d, J = 7.6 Hz, 1H), 8.17-8.12 (m, 1H), |
| | | | | 7.98-7.93 (m, 1H), 7.82 (t, J = 5.6 Hz, |
| | | | | 1H), 7.77 (d, J = 9.2 Hz, 1H), 7.68 (d, |
| | | | | J = 8.8 Hz, 2H), 7.58 (d, J = 7.6 Hz, 1H), |
| | | | | 7.48 (d, J = 8.4 Hz, 2H), 7.43 (s, 2H), |
| | | | | 7.42-7.38 (m, 2H), 7.38-7.35 (m, 2H), |
| | | | | 7.34-7.31 (m, 2H), 7.30 (d, J = 7.2 Hz, |
| | | | | 2H), 7.22 (t, J = 7.6 Hz, 2H), 7.16-7.12 |
| | | | | (m, 2H), 7.04-6.98 (m, 1H), 6.94-6.86 |
| | | | | (m, 1H), 6.82 (d, J = 8.8 Hz, 2H), 5.23 |
| | | | | (d, J = 4.0 Hz, 1H), 5.10 (d, J = 3.2 Hz, |
| | | | | 1H), 4.97-4.87 (m, 1H), 4.52 (d, J = 9.2 |
| | | | | Hz, 1H), 4.43 (t, J = 8.0 Hz, 1H), 4.33- |
| | | | | 4.25 (m, 2H), 4.06-3.97 (m, 1H), 3.90- |
| | | | | 3.80 (m, 1H), 3.70 (d, J = 13.2 Hz, 1H), |
| | | | | 3.60 (s, 2H), 3.29-3.26 (m, 1H), 3.14- |
| | | | | 3.08 (m, 1H), 3.07-3.00 (m, 1H), 2.76- |
| | | | | 2.62 (m, 2H), 2.61-2.54 (m, 2H), 2.47- |
| | | | | 2.45 (m, 3H), 2.27-2.20 (m, 1H), 2.14- |
| | | | | 2.07 (m, 1H), 2.00 (t, J = 7.2 Hz, 3H), |
| | | | | 1.88 (d, J = 9.2 Hz, 2H), 1.83-1.75 (m, |
| | | | | 2H), 1.70-1.63 (m, 1H), 1.45 (dd, J = |
| | | | | 6.4, 11.9 Hz, 4H), 1.38 (d, J = 6.8 Hz, |
| | | | | 3H), 1.24-1.18 (m, 2H), 1.09 (d, J = 9.5 |
| | | | | Hz, 1H), 1.03-0.99 (m, 1H), 0.93 (s, |
| | | | | 9H), 0.87-0.78 (m, 1H) |
| I-72 | OX | KT | 1469.4 | 11.98 (d, J = 1.6 Hz, 1 H), 8.99 (s, 1 H), |
| | | | | 8.37 (d, J = 7.6 Hz, 1 H), 8.14 (s, 1 H), |
| | | | | 7.95 (s, 1 H), 7.78 (d, J = 9.6 Hz, 1 H), |
| | | | | 7.69 (d, J = 9.2 Hz, 2 H), 7.58 (d, J = 7.6 |
| | | | | Hz, 1 H), 7.48 (d, J = 8.4 Hz, 2 H), 7.41- |
| | | | | 7.46 (m, 3 H), 7.35-7.40 (m, 3 H), 7.29- |
| | | | | 7.35 (m, 4 H), 7.24 (s, 2 H), 7.14 (d, J = |
| | | | | 7.2 Hz, 2 H), 6.96 (s, 2 H), 6.81 (d, J = |
| | | | | 8.8 Hz, 2 H), 5.23 (d, J = 4.4 Hz, 1 H), |
| | | | | 5.10 (d, J = 3.2 Hz, 1 H), 4.92 (t, J = 7.2 |
| | | | | Hz, 1 H), 4.48-4.54 (m, 1 H), 4.43 (t, J = |
| | | | | 8.0 Hz, 1 H), 4.24-4.34 (m, 2 H), 3.89- |
| | | | | 4.07 (m, 1 H), 3.85 (d, J = 12.4 Hz, 1 |
| | | | | H), 3.67 (s, 1 H), 3.61 (s, 2 H), 3.41 (s, 1 |
| | | | | H), 3.19-3.30 (m, 2 H), 2.88 (s, 2 H), |
| | | | | 2.73 (s, 1 H), 2.67 (s, 1 H), 2.54-2.60 |
| | | | | (m, 1 H), 2.44-2.47 (m, 3 H), 2.19 (t, J = |
| | | | | 7.2 Hz, 3 H), 2.08-2.14 (m, 1 H), 1.96- |
| | | | | 2.08 (m, 2 H), 1.89 (d, J = 13.2 Hz, 2 |
| | | | | H), 1.80 (td, J = 8.0, 4.31 Hz, 2 H), 1.60- |
| | | | | 1.70 (m, 1 H), 1.45 (dd, J = 14.4, 7.6 |
| | | | | Hz, 3 H), 1.38 (d, J = 7.2 Hz, 4 H), 1.23 |
| | | | | (d, J = 7.6 Hz, 2 H), 1.06-1.17 (m, 2 H), |
| | | | | 0.99 (s, 1 H), 0.93 (d, J = 3.6 Hz, 9 H), |
| | | | | 0.78-0.87 (m, 1 H) |
| I-73 | OY | KT | 1469.4 | 11.98 (d, J = 1.8 Hz, 1H), 8.99 (s, 1H), |
| | | | | 8.37 (d, J = 8.0 Hz, 1H), 8.15 (s, 1H), |
| | | | | 8.00-7.91 (m, 1H), 7.81-7.73 (m, 1H), |
| | | | | 7.68 (d, J = 8.8 Hz, 2H), 7.58 (d, J = 7.6 |
| | | | | Hz, 1H), 7.48 (d, J = 8.4 Hz, 2H), 7.46- |
| | | | | 7.42 (m, 3H), 7.40-7.37 (m, 2H), 7.34 |
| | | | | (d, J = 8.4 Hz, 2H), 7.30 (d, J = 9.2 Hz, |
| | | | | 3H), 7.26-7.19 (m, 2H), 7.17-7.11 (m, |
| | | | | 2H), 7.02-6.91 (m, 2H), 6.83 (d, J = 9.2 |
| | | | | Hz, 2H), 5.23 (d, J = 4.4 Hz, 1H), 5.10 |
| | | | | (s, 1H), 4.92 (quin, J = 7.2 Hz, 1H), |
| | | | | 4.56-4.47 (m, 1H), 4.42 (t, J = 8.0 Hz, 1H), |
| | | | | 4.33-4.23 (m, 2H), 4.10-3.82 (m, 2H), |
| | | | | 3.71 (dd, J = 1.6, 13.1 Hz, 1H), 3.60 (s, |
| | | | | 2H), 3.29-3.17 (m, 2H), 2.88 (s, 2H), |
| | | | | 2.73 (s, 2H), 2.70-2.64 (m, 2H), 2.46 (s, |
| | | | | 3H), 2.25-2.17 (m, 2H), 2.15-2.07 (m, |
| | | | | 2H), 2.05-1.97 (m, 2H), 1.95-1.86 (m, |
| | | | | 2H), 1.84-1.76 (m, 2H), 1.73-1.62 (m, |
| | | | | 1H), 1.37 (d, J = 6.8 Hz, 4H), 1.28-1.18 |
| | | | | (m, 2H), 1.16-1.06 (m, 2H), 1.05-0.97 |
| | | | | (m, 2H), 0.93 (d, J = 3.6 Hz, 9H), 0.88- |
| | | | | 0.75 (m, 1H) |
| I-76 | PJ | KT | 1482.5 | 11.57 (s, 1H), 8.98 (s, 1H), 8.37 (d, J = |
| | | | | 7.6 Hz, 1H), 8.09-7.92 (m, 2H), 7.84 (t, |
| | | | | J = 5.6 Hz, 1H), 7.79 (d, J = 9.2 Hz, 2H), |
| | | | | 7.54 (d, J = 8.8 Hz, 1H), 7.46-7.41 (m, |
| | | | | 3H), 7.39-7.31 (m, 5H), 7.04 (d, J = 8.4 |
| | | | | Hz, 2H), 6.85-6.72 (m, 1H), 6.66-6.60 |
| | | | | (m, 1H), 6.33 (s, 1H), 6.23 (s, 1H), 5.10 |
| | | | | (d, J = 3.6 Hz, 1H), 4.96-4.87 (m, 1H), |
| | | | | 4.51 (d, J = 9.2 Hz, 1H), 4.42 (t, J = 8.0 |
| | | | | Hz, 1H), 4.27 (d, J = 3.2 Hz, 1H), 3.64- |
| | | | | 3.56 (m, 2H), 3.44 (s, 1H), 3.28 (d, J = |
| | | | | 6.4 Hz, 4H), 3.07 (d, J = 6.0 Hz, 2H), |
| | | | | 3.02 (s, 4H), 2.72 (s, 2H), 2.45 (s, 3H), |
| | | | | 2.28-2.21 (m, 2H), 2.19 (s, 2H), 2.14 (d, |
| | | | | J = 9.2 Hz, 2H), 2.10 (d, J = 6.4 Hz, 1H), |
| | | | | 2.07-1.97 (m, 4H), 1.95 (s, 2H), 1.78 |
| | | | | (ddd, J = 4.8, 8.4, 12.8 Hz, 1H), 1.64- |
| | | | | 1.59 (m, 2H), 1.52-1.45 (m, 4H), 1.37 |
| | | | | (d, J = 7.2 Hz, 4H), 1.25-1.19 (m, 2H), |
| | | | | 0.92 (s, 15H) |
| I-78 | LB | OF | 1523.6 | 11.53-11.50 (m, 1H), 8.40-8.34 (m, |
| | | | | 1H), 8.28 (s, 2H), 7.96 (s, 1H), 7.94- |
| | | | | 7.90 (m, 1H), 7.85-7.79 (m, 1H), 7.73- |
| | | | | 7.67 (m, 1H), 7.58-7.54 (m, 1H), 7.44 |
| | | | | (s, 1H), 7.42 (s, 2H), 7.39 (s, 1H), 7.36 |
| | | | | (s, 1H), 7.34 (s, 1H), 7.27-7.24 (m, 1H), |
| | | | | 7.06 (d, J = 8.4 Hz, 2H), 6.95-6.89 (m, |
| | | | | 1H), 6.73-6.67 (m, 1H), 6.64-6.59 (m, |
| | | | | 1H), 6.30 (br s, 1H), 6.26 (s, 1H), 5.14- |
| | | | | 5.05 (m, 1H), 4.95-4.88 (m, 1H), 4.51 |
| | | | | (d, J = 9.6 Hz, 1H), 4.45-4.39 (m, 1H), |
| | | | | 4.31-4.24 (m, 1H), 3.61 (s, 2H), 3.00 (s, |
| | | | | 4H), 2.73 (s, 2H), 2.68 (s, 1H), 2.45 (s, |
| | | | | 3H), 2.34 (d, J = 3.2 Hz, 6H), 2.28 (d, |
| | | | | J = 6.8 Hz, 4H), 2.24 (d, J = 8.0 Hz, 2H), |
| | | | | 2.20 (d, J = 4.4 Hz, 6H), 2.06-1.98 (m, |
| | | | | 2H), 1.96 (s, 2H), 1.86-1.74 (m, 2H), |
| | | | | 1.74-1.63 (m, 5H), 1.44-1.34 (m, 6H), |
| | | | | 0.94 (d, J = 2.4 Hz, 15H) |
| I-79 | LB | PT | 1509.6 | 11.71-11.65 (m, 1H), 9.03-8.92 (m, |
| | | | | 1H), 8.37 (d, J = 8.0 Hz, 1H), 8.13 (s, |
| | | | | 1H), 8.03 (d, J = 2.4 Hz, 1H), 7.93-7.83 |
| | | | | (m, 2H), 7.55-7.41 (m, 6H), 7.40-7.33 |
| | | | | (m, 5H), 7.06-6.99 (m, 3H), 6.67 (d, J = |
| | | | | 9.2 Hz, 1H), 6.40 (s, 1H), 6.19 (s, 1H), |
| | | | | 5.10 (d, J = 3.6 Hz, 1H), 4.92 (t, J = 7.2 |
| | | | | Hz, 1H), 4.53-4.47 (m, 1H), 4.46-4.40 |
| | | | | (m, 1H), 4.28 (s, 1H), 3.62-3.58 (m, |
| | | | | 2H), 3.46 (s, 4H), 3.06 (s, 4H), 2.79- |
| | | | | 2.69 (m, 3H), 2.47-2.45 (m, 4H), 2.44- |
| | | | | 2.32 (m, 7H), 2.17 (d, J = 16.4 Hz, 7H), |
| | | | | 2.05-1.92 (m, 4H), 1.89-1.65 (m, 4H), |
| | | | | 1.38 (d, J = 6.8 Hz, 5H), 0.93 (d, J = 3.6 |
| | | | | Hz, 15H) |
| I-81 | LB | LG | 1566.4 | 11.65 (s, 1H), 8.98 (s, 1H), 8.37 (d, J = |
| | | | | 7.6 Hz, 1H), 8.10 (s, 1H), 8.00 (s, 1H), |
| | | | | 7.90-7.84 (m, 1H), 7.78 (d, J = 8.8 Hz, |
| | | | | 1H), 7.51-7.46 (m, 3H), 7.45-7.41 (m, |
| | | | | 3H), 7.39-7.32 (m, 5H), 7.04 (d, J = 8.4 |
| | | | | Hz, 2H), 6.98-6.91 (m, 1H), 6.65 (d, J = |
| | | | | 8.8 Hz, 1H), 6.38 (s, 1H), 6.20 (s, 1H), |
| | | | | 5.09 (d, J = 3.6 Hz, 1H), 4.91 (s, 1H), |
| | | | | 4.51 (d, J = 9.2 Hz, 1H), 4.42 (t, J = 8.0 |
| | | | | Hz, 1H), 4.27 (s, 1H), 3.60 (s, 2H), 3.48 |
| | | | | (s, 1H), 3.43 (d, J = 2.8 Hz, 4H), 3.04 (s, |
| | | | | 4H), 2.73 (s, 2H), 2.64-2.55 (m, 8H), |
| | | | | 2.53-2.52 (m, 3H), 2.45-2.45 (m, 3H), |
| | | | | 2.37 (s, 2H), 2.34-2.32 (m, 2H), 2.26 (d, |
| | | | | J = 7.2 Hz, 3H), 2.24 (s, 1H), 2.17 (dd, |
| | | | | J = 2.0, 6.4 Hz, 3H), 2.14 (d, J = 1.6 Hz, |
| | | | | 2H), 2.11 (d, J = 8.4 Hz, 1H), 2.00 (s, |
| | | | | 2H), 1.95 (s, 2H), 1.79 (dd, J = 4.0, 8.8, |
| | | | | 12.8 Hz, 2H), 1.73-1.68 (m, 2H), 1.45 |
| | | | | (s, 3H), 1.40 (s, 1H), 1.37 (d, J = 7.2 Hz, |
| | | | | 3H), 1.23 (s, 2H), 0.93 (d, J = 3.6 Hz, 9H) |
| I-82 | OU | KT | 1455.5 | 9.04 (s, 1H), 8.43 (d, J = 8.0 Hz, 1H), |
| | | | | 8.20 (d, J = 2.0 Hz, 1H), 8.01 (dd, J = 2.4, |
| | | | | 9.2 Hz, 1H), 7.90-7.81 (m, 2H), 7.74 (d, |
| | | | | J = 9.2 Hz, 2H), 7.64 (d, J = 7.2 Hz, 1H), |
| | | | | 7.56-7.48 (m, 5H), 7.47-7.38 (m, 5H), |
| | | | | 7.35 (d, J = 7.2 Hz, 3H), 7.28 (t, J = 7.6 |
| | | | | Hz, 2H), 7.22-7.17 (m, 2H), 7.08 (d, J = |
| | | | | 9.6 Hz, 1H), 7.00-6.94 (m, 1H), 6.88 (d, |
| | | | | J = 9.2 Hz, 2H), 5.29 (d, J = 4.4 Hz, 1H), |
| | | | | 5.16 (d, J = 3.2 Hz, 1H), 4.98 (t, J = 7.2 |
| | | | | Hz, 1H), 4.57 (d, J = 9.6 Hz, 1H), 4.48 (t, |
| | | | | J = 8.0 Hz, 1H), 4.39-4.31 (m, 2H), 4.07 |
| | | | | (d, J = 5.6 Hz, 1H), 3.91 (s, 1H), 3.74 (s, |
| | | | | 1H), 3.66 (s, 2H), 3.19-3.06 (m, 2H), |
| | | | | 2.81-2.69 (m, 2H), 2.68-2.61 (m, 2H), |
| | | | | 2.53-2.50 (m, 3H), 2.28 (dd, J = 7.2, |
| | | | | 14.5 Hz, 1H), 2.17 (d, J = 8.0 Hz, 1H), |
| | | | | 2.06 (t, J = 7.2 Hz, 3H), 1.94 (d, J = 9.2 |
| | | | | Hz, 2H), 1.89-1.80 (m, 2H), 1.77-1.69 |
| | | | | (m, 1H), 1.55-1.46 (m, 4H), 1.43 (d, J = |
| | | | | 7.2 Hz, 3H), 1.30-1.23 (m, 2H), 1.17- |
| | | | | 1.11 (m, 1H), 1.09-1.04 (m, 1H), 0.99 |
| | | | | (s, 9H), 0.89 (d, J = 2.8 Hz, 1H) |
| I-84 | PI | KT | 1522.6 | 11.86-11.64 (m, 1H), 11.60-11.11 (m, |
| | | | | 1H), 8.98 (s, 1H), 8.37 (d, J = 8.0 Hz, |
| | | | | 1H), 8.15 (s, 1H), 8.13 (s, 1H), 8.04 (d, |
| | | | | J = 2.4 Hz, 1H), 7.95-7.88 (m, 1H), 7.79 |
| | | | | (d, J = 9.2 Hz, 1H), 7.55 (s, 1H), 7.51 (t, |
| | | | | J = 2.8 Hz, 1H), 7.48 (d, J = 8.8 Hz, 1H), |
| | | | | 7.45-7.40 (m, 2H), 7.39-7.32 (m, 4H), |
| | | | | 7.28 (s, 1H), 7.08-7.00 (m, 3H), 6.72- |
| | | | | 6.63 (m, 1H), 6.44-6.38 (m, 1H), 6.18 |
| | | | | (s, 1H), 5.10 (d, J = 3.2 Hz, 1H), 4.96- |
| | | | | 4.87 (m, 1H), 4.51 (d, J = 9.2 Hz, 1H), |
| | | | | 4.45-4.33 (m, 2H), 4.27 (s, 1H), 3.92- |
| | | | | 3.79 (m, 1H), 3.65-3.54 (m, 2H), 3.27- |
| | | | | 3.23 (m, 2H), 3.06 (s, 4H), 2.94 (t, J = |
| | | | | 12.4 Hz, 1H), 2.81-2.69 (m, 2H), 2.45 |
| | | | | (s, 3H), 2.24 (td, J = 7.2, 14.4 Hz, 7H), |
| | | | | 2.16-2.10 (m, 3H), 2.03-1.93 (m, 3H), |
| | | | | 1.87-1.76 (m, 2H), 1.65 (t, J = 13.2 Hz, |
| | | | | 2H), 1.55-1.41 (m, 5H), 1.41-1.34 (m, |
| | | | | 5H), 1.27-1.21 (m, 2H), 1.14-1.06 (m, |
| | | | | 1H), 0.99 (d, J = 12.4 Hz, 1H), 0.92 (d, |
| | | | | J = 4.4 Hz, 15H) |
| I-85 | PZ | QA | 1478.6 | 11.66 (s, 1H), 8.35 (d, J = 8.0 Hz, 1H), |
| | | | | 8.15-8.09 (m, 1H), 8.01 (d, J = 2.4 Hz, |
| | | | | 1H), 7.92-7.85 (m, 1H), 7.77 (d, J = 9.2 |
| | | | | Hz, 1H), 7.52-7.46 (m, 3H), 7.40 (d, J = |
| | | | | 8.0 Hz, 2H), 7.34 (d, J = 8.4 Hz, 3H), |
| | | | | 7.28 (d, J = 8.0 Hz, 2H), 7.03 (d, J = 8.4 |
| | | | | Hz, 2H), 7.01-6.94 (m, 1H), 6.66 (dd, J = |
| | | | | 1.2, 8.4 Hz, 1H), 6.39 (d, J = 1.2 Hz, |
| | | | | 1H), 6.19 (s, 1H), 5.11 (d, J = 3.6 Hz, |
| | | | | 1H), 4.90-4.82 (m, 1H), 4.49 (d, J = 9.2 |
| | | | | Hz, 1H), 4.39 (t, J = 8.0 Hz, 1H), 4.26 (s, |
| | | | | 1H), 4.12-4.08 (m, 1H), 3.58 (s, 4H), |
| | | | | 3.05 (s, 4H), 2.73 (s, 2H), 2.45-2.35 (m, |
| | | | | 5H), 2.27-2.23 (m, 3H), 2.22-2.10 (m, |
| | | | | 8H), 2.00-1.94 (m, 3H), 1.77-1.69 (m, |
| | | | | 3H), 1.52-1.41 (m, 5H), 1.41-1.35 (m, |
| | | | | 3H), 1.32 (d, J = 7.2 Hz, 3H), 1.24 (q, |
| | | | | J = 7.2 Hz, 3H), 0.92 (s, 16H) |
| I-86 | LB | LP | 1579.6 | 11.69 (s, 1H), 8.99 (s, 1H), 8.37 (d, J = |
| | | | | 8.0 Hz, 1H), 8.14 (s, 1H), 8.03 (d, J = 2.4 |
| | | | | Hz, 1H), 7.91 (d, J = 9.6 Hz, 1H), 7.78 |
| | | | | (d, J = 9.6 Hz, 1H), 7.53-7.47 (m, 3H), |
| | | | | 7.46-7.42 (m, 2H), 7.40-7.33 (m, 5H), |
| | | | | 7.07-7.00 (m, 3H), 6.70-6.64 (m, 1H), |
| | | | | 6.40 (d, J = 1.2 Hz, 1H), 6.19 (s, 1H), |
| | | | | 5.10 (d, J = 3.2 Hz, 1H), 4.97-4.89 (m, |
| | | | | 1H), 4.52 (d, J = 9.2 Hz, 1H), 4.43 (t, J = |
| | | | | 8.0 Hz, 1H), 4.28 (d, J = 2.4 Hz, 1H), |
| | | | | 3.61 (s, 2H), 3.44 (s, 3H), 3.38 (d, J = 5.6 |
| | | | | Hz, 2H), 3.06 (s, 4H), 2.78-2.71 (m, |
| | | | | 2H), 2.46 (s, 4H), 2.43-2.36 (m, 4H), |
| | | | | 2.29-2.23 (m, 4H), 2.22-2.13 (m, 7H), |
| | | | | 2.04-1.93 (m, 4H), 1.80-1.70 (m, 3H), |
| | | | | 1.51-1.42 (m, 5H), 1.38 (d, J = 7.2 Hz, |
| | | | | 4H), 1.25 (s, 7H), 0.93 (d, J = 3.6 Hz, |
| | | | | 15H) |
| I-88 | PC | KT | 1534.3 | 8.98 (s, 1H), 8.37 (s, 1H), 8.07 (s, 1H), |
| | | | | 7.95 (s, 1H), 7.78 (s, 1H), 7.67 (d, J = 8.4 |
| | | | | Hz, 2H), 7.58 (d, .J = 5.6 Hz, 1H), 7.48 |
| | | | | (d, J = 7.6 Hz, 2H), 7.43 (d, J = 6.8 Hz, |
| | | | | 3H), 7.39-7.30 (m, 10H), 7.23-7.11 (m, |
| | | | | 3H), 6.92-6.84 (m, 1H), 6.73 (s, 2H), |
| | | | | 5.21 (s, 1H), 5.10 (s, 1H), 4.91 (s, 1H), |
| | | | | 4.51 (d, J = 8.8 Hz, 1H), 4.41 (s, 1H), |
| | | | | 4.29 (s, 2H), 4.03-3.96 (m, 1H), 3.72 |
| | | | | (dd, J = 2.8, 9.2 Hz, 1H), 3.59 (d, J = 1.2 |
| | | | | Hz, 4H), 3.39 (s, 2H), 3.23 (d, J = 3.2 Hz, |
| | | | | 3H), 2.98 (s, 3H), 2.66 (s, 2H), 2.45 (s, |
| | | | | 3H), 2.25 (s, 2H), 2.13 (s, 2H), 2.01 (s, |
| | | | | 2H), 1.91 (s, 1H), 1.79 (s, 1H), 1.47 (s, |
| | | | | 4H), 1.37 (d, J = 5.6 Hz, 3H), 1.23 (s, |
| | | | | 4H), 1.13-1.08 (m. 1H), 0.93 (s, 9H) |
| I-89 | A | PV | 1557.5 | 11.68-11.60 (m, 1H), 11.58-11.34 (m, |
| | | | | 1H), 9.01 (s, 1H), 8.41 (d, J = 8.0 Hz, |
| | | | | 1H), 8.14-8.07 (m, 1H), 8.02 (s, 1H), |
| | | | | 7.88-7.78 (m, 2H), 7.53 (d, J = 8.8 Hz, |
| | | | | 1H), 7.50-7.46 (m, 2H), 7.45 (s, 2H), |
| | | | | 7.42-7.39 (m, 2H), 7.37 (d, J = 8.4 Hz, |
| | | | | 2H), 7.07 (d, J = 8.4 Hz, 2H), 6.96-6.87 |
| | | | | (m, 1H), 6.83 (d, J = 8.8 Hz, 2H), 6.67 |
| | | | | (d, J = 9.2 Hz, 3H), 6.38 (s, 1H), 6.23 ( s, |
| | | | | 1H), 5.13 (d, J = 3.6 Hz, 1H), 4.98-4.91 |
| | | | | (m, 1H), 4.56-4.51 (m, 1H), 4.48-4.43 |
| | | | | (m, 1H), 4.31 (s, 1H), 3.63 (s, 2H), 3.47 |
| | | | | (d, J = 11.2 Hz, 2H), 3.27 (t, J = 6.8 Hz, |
| | | | | 3H), 3.19-3.13 (m, 1H), 3.06 (s, 3H), |
| | | | | 2.78 (s, 3H), 2.75 (s, 2H), 2.48 (s, 3H), |
| | | | | 2.23-2.15 (m, 6H), 2.07-2.01 (m, 1H), |
| | | | | 1.98 (s, 2H), 1.82-1.73 (m, 4H), 1.71- |
| | | | | 1.67 (m, 2H), 1.41 (s, 3H), 1.39 (s, 2H), |
| | | | | 1.27 (d, J = 7.2 Hz, 6H), 0.96 (d, J = 8.8 |
| | | | | Hz, 14H) |
| I-96 | QJ | KT | 1551.6 | 11.74-11.49 (m, 1H), 9.01-8.94 (m, |
| | | | | 1H), 8.42-8.31 (m, 1H), 8.13 (s, 1H), |
| | | | | 8.04-7.90 (m, 2H), 7.90-7.82 (m, 1H), |
| | | | | 7.81-7.72 (m, 1H), 7.52 (d, J = 8.4 Hz, |
| | | | | 1H), 7.46 (s, 1H), 7.45-7.40 (m, 3H), |
| | | | | 7.39-7.29 (m, 5H), 7.04 (d, J = 8.4 Hz, |
| | | | | 2H), 6.99-6.81 (m, 1H), 6.67-6.61 (m, |
| | | | | 1H), 6.40-6.30 (m, 1H), 6.24-6.18 (m, |
| | | | | 1H), 5.09 (d, J = 3.6 Hz, 1H), 4.96-4.87 |
| | | | | (m, 1H), 4.55-4.47 (m, 1H), 4.46-4.36 |
| | | | | (m, 1H), 4.27-4.27 (m, 1H), 4.33-4.25 |
| | | | | (m, 1H), 4.24-4.11 (m, 1H), 3.63-3.57 |
| | | | | (m, 2H), 3.09-2.95 (m, 6H), 2.76-2.70 |
| | | | | (m, 3H), 2.54 (s, 2H), 2.46-2.45 (m, |
| | | | | 3H), 2.25-2.10 (m, 10H), 2.08 (s, 1H), |
| | | | | 2.03 (t, J = 6.8 Hz, 3H), 1.98 (s, 1H), |
| | | | | 1.97-1.94 (m, 2H), 1.82-1.74 (m, 3H), |
| | | | | 1.53-1.43 (m, 5H), 1.42-1.33 (m, 6H), |
| | | | | 1.25-1.17 (m, 3H), 0.94-0.91 (m, 15H) |
| I-99 | OK | KT | 1562.5 | 11.64 (s, 1H), 9.00-8.93 (m, 1H), 8.40- |
| | | | | 8.30 (m, 1H), 8.07-7.99 (m, 1H), 7.98- |
| | | | | 7.92 (m, 1H), 7.85-7.71 (m, 2H), 7.61- |
| | | | | 7.54 (m, 1H), 7.49-7.34 (m, 10H), 7.35- |
| | | | | 7.27 (m, 1H), 7.10 (d, J = 8.4 Hz, 1H), |
| | | | | 7.07-7.02 (m, 1H), 6.95-6.89 (m, 1H), |
| | | | | 6.86-6.76 (m, 1H), 6.68-6.61 (m, 1H), |
| | | | | 6.36-6.22 (m, 2H), 5.13 (br d, J = 3.2 |
| | | | | Hz, 1H), 4.97-4.87 (m, 1H), 4.55-4.47 |
| | | | | (m, 1H), 4.46-4.37 (m, 1H), 4.32-4.24 |
| | | | | (m, 1H), 3.64-3.54 (m, 6H), 3.19-3.00 |
| | | | | (m, 10H), 2.90-2.76 (m, 2H), 2.57 (s, |
| | | | | 6H), 2.45 (s, 3H), 2.37-2.31 (m, 6H), |
| | | | | 2.26-2.21 (m, 1H), 2.17-2.16 (m, 1H), |
| | | | | 2.21-2.04 (m, 2H), 2.03-1.96 (m, 1H), |
| | | | | 1.85-1.76 (m, 3H), 1.58-1.41 (m, 5H), |
| | | | | 1.37 (d, J = 7.2 Hz, 3H), 1.33-1.18 (m, |
| | | | | 3H), 0.93 (s, 9H) |
| I- | A | PR | 1488.5 | 11.65 (s, 1H), 8.99-8.97 (m, 1H), 8.38 |
| 101 | | | | (d, J = 7.6 Hz, 1H), 8.10 (s, 1H), 8.02 (d, |
| | | | | J = 2.4 Hz, 1H), 7.89-7.80 (m, 2H), 7.51 |
| | | | | (d, J = 8.8 Hz, 1H), 7.48 (d, J = 2.8 Hz, |
| | | | | 2H), 7.45-7.40 (m, 3H), 7.39-7.33 (m, |
| | | | | 5H), 7.32-7.28 (m, 1H), 7.04 (d, J = 8.4 |
| | | | | Hz, 2H), 6.99-6.93 (m, 1H), 6.90 (d, J = |
| | | | | 8.4 Hz, 2H), 6.66 (d, J = 8.8 Hz, 1H), |
| | | | | 6.54 (d, J = 8.4 Hz, 2H), 6.37 (s, 1H), |
| | | | | 6.21 (s, 1H), 5.54 (s, 1H), 5.11 (d, J = 3.6 |
| | | | | Hz, 1H), 4.94-4.86 (m, 1H), 4.53 (d, J = |
| | | | | 9.2 Hz, 1H), 4.43 (t, J = 8.0 Hz, 1H), 4.28 |
| | | | | (s, 1H), 3.61 (s, 3H), 3.53-3.45 (m, 3H), |
| | | | | 3.24-3.17 (m, 3H), 3.04 (s, 4H), 2.73 (s, |
| | | | | 2H), 2.46-2.45 (m, 3H), 2.42-2.37 (m, |
| | | | | 2H), 2.19 (s, 4H), 2.15 (s, 2H), 2.04- |
| | | | | 1.98 (m, 1H), 1.95 (s, 2H), 1.80 (dt, J = |
| | | | | 4.0, 8.8 Hz, 1H), 1.74-1.66 (m, 2H), |
| | | | | 1.37 (d, J = 7.2 Hz, 3H), 0.93 (d, J = 7.2 |
| | | | | Hz, 15H) |
| I- | LB | PY | 1495.6 | 11.69 (s, 1H), 8.35 (d, J = 8.4 Hz, 1H), |
| 102^{c} | | | | 8.13 (s, 1H), 8.03 (d, J = 2.4 Hz, 1H), |
| | | | | 7.93-7.87 (m, 1H), 7.81 (d, J = 9.2 Hz, |
| | | | | 1H), 7.54-7.46 (m, 3H), 7.43-7.37 (m, |
| | | | | 3H), 7.32 (dd, J = 8.4, 15.2 Hz, 5H), 7.04 |
| | | | | (d, J = 8.4 Hz, 3H), 6.67 (d, J = 9.2 Hz, |
| | | | | 1H), 6.39 (s, 1H), 6.18 (s, 1H), 5.10 (d, |
| | | | | J = 3.6 Hz, 1H), 4.86-4.79 (m, 1H), 4.74 |
| | | | | (t, J = 6.0 Hz, 1H), 4.51 (d, J = 9.6 Hz, |
| | | | | 1H), 4.44 (t, J = 8.0 Hz, 1H), 4.26 (s, |
| | | | | 1H), 4.15-4.12 (m, 1H), 3.60 (s, 2H), |
| | | | | 3.58-3.51 (m, 2H), 3.44 (s, 3H), 3.38 (s, |
| | | | | 1H), 3.06 (s, 4H), 2.77-2.71 (m, 2H), |
| | | | | 2.43-2.35 (m, 4H), 2.28-2.22 (m, 4H), |
| | | | | 2.21-2.06 (m, 8H), 2.02-1.92 (m, 4H), |
| | | | | 1.81-1.70 (m, 3H), 1.51-1.43 (m, 4H), |
| | | | | 1.38 (t, J = 6.0 Hz, 2H), 1.30-1.18 (m, |
| | | | | 3H), 0.92 (s, 15H) |
| I- | QB | QA | 1496.6 | 11.67 (br s, 1H), 8.39-8.34 (m, 1H), |
| 105 | | | | 8.12 (br s, 1H), 8.02 (br d, J = 2.4 Hz, |
| | | | | 1H), 7.93-7.85 (m, 1H), 7.81-7.75 (m, |
| | | | | 1H), 7.56-7.44 (m, 5H), 7.34 (d, J = 8.4 |
| | | | | Hz, 2H), 7.20 (d, J = 10.4 Hz, 1H), 7.13 |
| | | | | (d, J = 8.0 Hz, 1H), 7.04 (d, J = 8.4 Hz, |
| | | | | 2H), 7.00 (br dd, J = 2.0, 7.6 Hz, 1H), |
| | | | | 6.66 (br d, J = 9.2 Hz, 1H), 6.39 (br s, |
| | | | | 1H), 6.19 (s, 1H), 5.09 (d, J = 3.6 Hz, |
| | | | | 1H), 4.91-4.85 (m, 1H), 4.50 (d, J = 9.2 |
| | | | | Hz, 1H), 4.43 (s, 1H), 4.41-4.35 (m, |
| | | | | 1H), 4.27 (br s, 1H), 3.62-3.57 (m, 2H), |
| | | | | 3.43 (br s, 4H), 3.05 (br s, 4H), 2.74 (br |
| | | | | d, J = 4.4 Hz, 2H), 2.41-2.35 (m, 4H), |
| | | | | 2.28-2.23 (m, 4H), 2.21 (br s, 1H), 2.16 |
| | | | | (ddd, J = 2.4, 6.0, 8.0 Hz, 5H), 2.12 (br |
| | | | | s, 1H), 2.07 (s, 1H), 2.03-1.98 (m, 1H), |
| | | | | 1.95 (br s, 2H), 1.78-1.70 (m, 3H), 1.51- |
| | | | | 1.43 (m, 5H), 1.38 (br t, J = 6.0 Hz, 2H), |
| | | | | 1.33 (d, J = 7.2 Hz, 3H), 1.27-1.22 (m, |
| | | | | 3H), 0.92 (s, 15H) |
| I- | QI | KT | 1565.6 | 11.64 (br s, 1H), 8.98 (s, 1H), 8.37 (d, |
| 106 | | | | J = 8.0 Hz, 1H), 8.10 (s, 1H), 8.01 (d, J = |
| | | | | 2.4 Hz, 1H), 7.88 (br d, J = 8.8 Hz, 1H), |
| | | | | 7.79 (br d, J = 9.2 Hz, 1H), 7.54-7.41 |
| | | | | (m, 5H), 7.41-7.28 (m, 5H), 7.04 (d, J = |
| | | | | 8.4 Hz, 2H), 6.94 (br d, J = 8.8 Hz, 1H), |
| | | | | 6.71-6.61 (m, 1H), 6.41-6.35 (m, 1H), |
| | | | | 6.20 (s, 1H), 5.11 (d, J = 3.6 Hz, 1H), |
| | | | | 4.98-4.87 (m, 1H), 4.52 (d, J = 9.2 Hz, |
| | | | | 1H), 4.43 (t, J = 8.0 Hz, 1H), 4.28 (br d, |
| | | | | J = 1.6 Hz, 1H), 3.61 (br s, 2H), 3.53 (br |
| | | | | s, 2H), 3.50-3.43 (m, 3H), 3.04 (br s, |
| | | | | 4H), 2.92-2.62 (m, 8H), 2.46 (s, 3H), |
| | | | | 2.30-2.10 (m, 11H), 2.05-1.98 (m, 1H), |
| | | | | 1.95 (br s, 2H), 1.87 (br d, J = 2.0 Hz, |
| | | | | 1H), 1.83-1.71 (m, 4H), 1.57-1.43 (m, |
| | | | | 5H), 1.41-1.35 (m, 5H), 1.30-1.22 (m, |
| | | | | 2H), 0.97-0.93 (m, 9H), 0.93 (br s, 6H) |
| I- | KS | MK | 1510.5 | 8.98 (s, 1H), 8.37 (d, J = 8.0 Hz, 1H), |
| 109 | | | | 8.13 (d, J = 1.6 Hz, 1H), 7.95 (dd, J = 1.6, |
| | | | | 9.2 Hz, 1H), 7.80 (d, J = 9.2 Hz, 1H), |
| | | | | 7.67 (d, J = 8.8 Hz, 2H), 7.58 (d, J = 7.6 |
| | | | | Hz, 1H), 7.47 (d, J = 8.4 Hz, 2H), 7.45- |
| | | | | 7.40 (m, 3H), 7.39-7.34 (m, 3H), 7.34- |
| | | | | 7.29 (m, 4H), 7.26 (t, J = 7.6 Hz, 2H), |
| | | | | 7.19-7.11 (m, 2H), 7.03 (d, J = 9.2 Hz, |
| | | | | 1H), 6.98-6.89 (m, 1H), 6.80 (d, J = 8.8 |
| | | | | Hz, 2H), 5.23 (d, J = 4.4 Hz, 1H), 5.10 |
| | | | | (d, J = 3.6 Hz, 1H), 4.95-4.87 (m, 1H), |
| | | | | 4.55-4.48 (m, 1H), 4.42 (t, J = 8.0 Hz, |
| | | | | 1H), 4.32-4.24 (m, 2H), 4.15-4.04 (m, |
| | | | | 1H), 3.83 (d, J = 12.4 Hz, 1H), 3.69- |
| | | | | 3.56 (m, 3H), 3.38 (d, J = 5.2 Hz, 2H), |
| | | | | 3.37-3.35 (m, 2H), 3.27 (s, 2H), 2.66 (d, |
| | | | | J = 4.4 Hz, 1H), 2.58-2.51 (m, 2H), 2.45 |
| | | | | (s, 3H), 2.39 (s, 2H), 2.30-2.20 (m, 4H), |
| | | | | 2.19-2.03 (m, 2H), 2.03-1.94 (m, 2H), |
| | | | | 1.88 (d, J = 12.0 Hz, 1H), 1.79 (ddd, J = |
| | | | | 4.4, 8.4, 12.4 Hz, 2H), 1.70-1.61 (m, |
| | | | | 1H), 1.58-1.39 (m, 5H), 1.37 (d, J = 7.2 |
| | | | | Hz, 3H), 1.13-1.05 (m, 1H), 1.00 (d, J = |
| | | | | 11.6 Hz, 1H), 0.92 (s, 9H), 0.86-0.76 |
| | | | | (m, 1H) |
| I- | KS | OF | 1496.3 | 11.91 (s, 1H), 11.76 (s, 1H), 10.88 (d, J = |
| 110 | | | | 2.8 Hz, 1H), 9.17 (s, 2H), 8.19 (d, J = |
| | | | | 2.0 Hz, 1H), 8.09 (d, J = 2.4 Hz, 1H), |
| | | | | 8.01-7.92 (m, 1H), 7.67 (d, J = 2.0 Hz, |
| | | | | 1H), 7.58-7.48 (m, 3H), 7.41-7.33 (m, |
| | | | | 3H), 7.09 (d, J = 8.0 Hz, 2H), 6.72 (d, J = |
| | | | | 8.8 Hz, 1H), 6.46 (s, 1H), 6.26 (s, 1H), |
| | | | | 5.75 (s, 1H), 3.76 (d, J = 6.8 Hz, 2H), |
| | | | | 3.62 (d, J = 13.6 Hz, 2H), 3.53 (s, 2H), |
| | | | | 3.38-3.21 (m, 5H), 3.04 (d, J = 5.6 Hz, |
| | | | | 2H), 2.68 (d, J = 10.0 Hz, 2H), 2.56 (t, |
| | | | | J = 5.2 Hz, 3H), 2.36 (s, 2H), 2.00 (s, 2H), |
| | | | | 1.43 ( t, J = 6.0 Hz, 2H), 1.25-1.16 (m, |
| | | | | 1H), 0.93 (s, 6H) |
| I- | NY | KT | 1592.2 | 8.98 (s, 1H), 8.50 (t, J = 5.6 Hz, 1H), 8.13 |
| 111 | | | | (s, 1H), 8.05 (d, J = 1.2 Hz, 1H), 7.90 (d, |
| | | | | J = 9.2 Hz, 1H), 7.68 (d, J = 8.8 Hz, 2H), |
| | | | | 7.55 (d, J = 7.6 Hz, 1H), 7.41 (d, J = 7.6 |
| | | | | Hz, 1H), 7.35-7.32 (m, 2H), 7.30-7.25 |
| | | | | (m, 4H), 7.18-7.14 (m, 2H), 7.02 (s, |
| | | | | 1H), 6.96 (d, J = 7.6 Hz, 2H), 6.85 (d, |
| | | | | J = 8.8 Hz, 1H), 6.75 (d, J = 8.8 Hz, 2H), |
| | | | | 6.71-6.67 (m, 1H), 5.17 (d, J = 3.6 Hz, |
| | | | | 1H), 4.59 (d, J = 9.2 Hz, 1H), 4.52 (t, J = |
| | | | | 8.4 Hz, 1H), 4.34 (d, J = 4.8 Hz, 2H), |
| | | | | 4.28 (d, J = 6.4 Hz, 1H), 4.22 (d, J = 4.8 |
| | | | | Hz, 1H), 4.18-4.14 (m, 2H), 4.02-3.97 |
| | | | | (m, 1H), 3.68-3.57 (m, 4H), 3.13-3.06 |
| | | | | (m, 4H), 3.00 (s, 4H), 2.84-2.75 (m, |
| | | | | 2H), 2.54 (s, 2H), 2.45 (s, 3H), 2.12- |
| | | | | 2.03 (m, 4H), 1.95-1.88 (m, 4H), 1.76- |
| | | | | 1.68 (m, 2H), 1.63-1.56 (m, 2H), 1.52- |
| | | | | 1.31 (m, 12H), 1.26-1.18 (m, 4H), 0.94 |
| | | | | (s, 9H), 0.69-0.55 (m, 3H) |
| I- | OQ | KT | 1597.6 | 8.99 (s, 1H), 8.37 (d, J = 7.6 Hz, 1H), |
| 113 | | | | 8.08 (d, J = 1.6 Hz, 1H), 7.96 (dd, J = 1.6, |
| | | | | 9.0 Hz, 1H), 7.80 (d, J = 9.2 Hz, 1H), |
| | | | | 7.67 (d, J = 8.8 Hz, 2H), 7.47-7.42 (m, |
| | | | | 4H), 7.39 (s, 1H), 7.38-7.34 (m, 3H), |
| | | | | 7.33-7.27 (m, 4H), 7.22-7.17 (m, 1H), |
| | | | | 7.15 (d, J = 2.0 Hz, 1H), 7.05-6.98 (m, |
| | | | | 1H), 6.94-6.87 (m, 2H), 6.78-6.68 (m, |
| | | | | 3H), 5.17 (d, J = 4.4 Hz, 1H), 5.11 (d, |
| | | | | J = 3.6 Hz, 1H), 4.97-4.87 (m, 1H), 4.52 |
| | | | | (d, J = 9.2 Hz, 1H), 4.47-4.39 (m, 1H), |
| | | | | 4.36-4.24 (m, 2H), 4.08-3.98 (m, 1H), |
| | | | | 3.78-3.70 (m, 1H), 3.61 (d, J = 2.8 Hz, |
| | | | | 9H), 3.22-3.11 (m, 6H), 3.07-2.82 (m, |
| | | | | 4H), 2.61-2.56 (m, 2H), 2.46 (s, 3H), |
| | | | | 2.38-2.31 (m, 3H), 2.30-2.18 (m, 2H), |
| | | | | 2.13 (td, J = 7.2, 14.4 Hz, 2H), 2.08- |
| | | | | 1.96 (m, 3H), 1.94-1.88 (m, 1H), 1.80 |
| | | | | (ddd, J = 4.4, 8.4, 12.8 Hz, 1H), 1.63- |
| | | | | 1.57 (m, 1H), 1.56-1.44 (m, 5H), 1.38 |
| | | | | (d, J = 7.2 Hz, 3H), 1.33-1.22 (m, 3H), |
| | | | | 1.15-1.09 (m, 1H), 1.03 (d, J = 9.6 Hz, |
| | | | | 1H), 0.96-0.92 (m, 9H), 0.87-0.80 (m, |
| | | | | 1H |
| I- | OS | KT | 1597.6 | 8.98 (s, 1H), 8.37 (d, J = 7.8 Hz, 1H), |
| 114 | | | | 8.08 (s, 1H), 7.98-7.94 (m, 1H), 7.80 (d, |
| | | | | J = 9.2 Hz, 1H), 7.67 (d, J = 8.8 Hz, 2H), |
| | | | | 7.46-7.42 (m, 4H), 7.39-7.39 (m, 1H), |
| | | | | 7.37-7.34 (m, 3H), 7.32-7.28 (m, 4H), |
| | | | | 7.22-7.19 (m, 1H), 7.15-7.13 (m, 1H), |
| | | | | 7.03-7.00 (m, 1H), 6.91 (dd, J = 2.0, 8.6 |
| | | | | Hz, 2H), 6.77-6.69 (m, 3H), 5.18 (d, J = |
| | | | | 4.4 Hz, 1H), 5.11 (d, J = 3.6 Hz, 1H), |
| | | | | 4.94-4.89 (m, 1H), 4.52 (d, J = 9.2 Hz, |
| | | | | 1H), 4.45-4.40 (m, 1H), 4.33-4.27 (m, |
| | | | | 2H), 4.07-3.98 (m, 1H), 3.74-3.70 (m, |
| | | | | 1H), 3.61 (s, 9H), 3.20 (d, J = 5.6 Hz, |
| | | | | 6H), 3.17-3.12 (m, 4H), 2.53 (s, 2H), |
| | | | | 2.46 (s, 3H), 2.35-2.32 (m, 3H), 2.29- |
| | | | | 2.22 (m, 2H), 2.14 (dd, J = 6.8, 14.8 Hz, |
| | | | | 2H), 2.06-1.98 (m, 3H), 1.92-1.88 (m, |
| | | | | 1H), 1.80 (td, J = 4.4, 8.8 Hz, 1H), 1.62- |
| | | | | 1.57 (m, 1H), 1.51 (dd, J = 7.2, 15.2 Hz, |
| | | | | 5H), 1.39-1.36 (m, 3H), 1.30-1.24 (m, |
| | | | | 3H), 1.14-1.09 (m, 1H), 1.05-1.01 (m, |
| | | | | 1H), 0.94 (s, 9H), 0.85 (dd, J = 4.4, 8.0 |
| | | | | Hz, 1H) |
| I- | KS | LG | 1538.5 | 12.39-11.09 (m, 1H), 8.98 (s, 1H), 8.38 |
| 120 | | | | (d, J = 7.6 Hz, 1H), 8.13 (s, 1H), 7.95 |
| | | | | (dd, J = 2.0, 9.2 Hz, 1H), 7.79 (d, J = 9.2 |
| | | | | Hz, 1H), 7.67 (d, J = 8.8 Hz, 2H), 7.58 |
| | | | | (d, J = 7.2 Hz, 1H), 7.47 (d, J = 8.4 Hz, |
| | | | | 2H), 7.45-7.40 (m, 3H), 7.38 (s, 1H), |
| | | | | 7.37-7.34 (m, 2H), 7.34-7.29 (m, 4H), |
| | | | | 7.25 (t, J = 7.6 Hz, 2H), 7.17 (d, J = 7.2 |
| | | | | Hz, 1H), 7.15-7.12 (m, 1H), 7.04 (d, J = |
| | | | | 9.2 Hz, 1H), 6.94 (d, J = 8.0 Hz, 1H), |
| | | | | 6.80 (d, J = 9.2 Hz, 2H), 5.23 (d, J = 4.4 |
| | | | | Hz, 1H), 5.10 (d, J = 3.6 Hz, 1H), 4.91 |
| | | | | (quin, J = 7.2 Hz, 1H), 4.54-4.49 (m, |
| | | | | 1H), 4.42 (t, J = 8.0 Hz, 1H), 4.33-4.23 |
| | | | | (m, 2H), 4.14-4.05 (m, 1H), 3.83 (d, J = |
| | | | | 12.0 Hz, 1H), 3.69-3.57 (m, 3H), 3.46- |
| | | | | 3.40 (m, 1H), 3.39-3.37 (m, 1H), 3.36- |
| | | | | 3.35 (m, 2H), 3.32-3.30 (m, 4H), 3.30- |
| | | | | 3.27 (m, 1H), 2.70-2.63 (m, 1H), 2.57- |
| | | | | 2.53 (m, 1H), 2.52 (s, 1H), 2.46-2.43 |
| | | | | (m, 4H), 2.27-2.17 (m, 4H), 2.10 (d, J = |
| | | | | 7.4 Hz, 1H), 2.04-1.95 (m, 2H), 1.88 (d, |
| | | | | J = 11.6 Hz, 1H), 1.85-1.72 (m, 2H), |
| | | | | 1.70-1.61 (m, 1H), 1.50-1.40 (m, 4H), |
| | | | | 1.37 (d, J = 6.8 Hz, 3H), 1.23 (s, 4H), |
| | | | | 1.13-1.05 (m, 1H), 1.00 (d, J = 12.8 Hz, |
| | | | | 1H), 0.93 (s, 9H), 0.81 (dt, J = 8.8, 12.0 |
| | | | | Hz, 1H) |
| I- | KS | LP | 1552.5 | 8.98 (s, 1H), 8.37 (d, J = 7.6 Hz, 1H), |
| 121 | | | | 8.11 (d, J = 1.6 Hz, 1H), 7.94 (dd, J = 1.6, |
| | | | | 9.2 Hz, 1H), 7.78 (d, J = 9.2 Hz, 1H), |
| | | | | 7.66 (d, J = 8.8 Hz, 2H), 7.57 (d, J = 7.6 |
| | | | | Hz, 1H), 7.47 (d, J = 8.4 Hz, 2H), 7.44- |
| | | | | 7.42 (m, 2H), 7.38 (s, 1H), 7.38-7.29 |
| | | | | (m, 6H), 7.29-7.24 (m, 2H), 7.19-7.15 |
| | | | | (m, 1H), 7.13 (d, J = 7.6 Hz, 1H), 6.99 |
| | | | | (d, J = 9.2 Hz, 1H), 6.89 (dd, J = 2.0, 3.6 |
| | | | | Hz, 1H), 6.78 (d, J = 8.8 Hz, 2H), 5.23 |
| | | | | (d, J = 4.4 Hz, 1H), 5.10 (d, J = 3.2 Hz, |
| | | | | 1H), 4.95-4.87 (m, 1H), 4.53-4.48 (m, |
| | | | | 1H), 4.42 (t, J = 8.0 Hz, 1H), 4.33-4.24 |
| | | | | (m, 2H), 4.12-4.03 (m, 1H), 3.84-3.76 |
| | | | | (m, 1H), 3.69-3.56 (m, 4H), 3.31-3.25 |
| | | | | (m, 4H), 2.69-2.56 (m, 2H), 2.45 (s, |
| | | | | 3H), 2.37 (s, 2H), 2.34-2.30 (m, 1H), |
| | | | | 2.28 (d, J = 2.4 Hz, 1H), 2.25-2.17 (m, |
| | | | | 4H), 2.17-2.05 (m, 2H), 2.04-1.95 (m, |
| | | | | 2H), 1.88 (d, J = 12.0 Hz, 1H), 1.83- |
| | | | | 1.74 (m, 2H), 1.68-1.59 (m, 1H), 1.47- |
| | | | | 1.39 (m, 4H), 1.37 (d, J = 7.2 Hz, 3H), |
| | | | | 1.28-1.17 (m, 8H), 1.13-0.97 (m, 3H), |
| | | | | 0.93 (s, 9H), 0.86-0.77 (m, 1H) |
| I- | MW | KT | 1595.6 | 8.98 (s, 1H), 8.39-8.33 (m, 1H), 8.08 (d, |
| 122 | | | | J = 1.8 Hz, 1H), 7.98-7.95 (m, 1H), 7.79 |
| | | | | (d, J = 9.2 Hz, 1H), 7.66 (d, J = 8.8 Hz, |
| | | | | 2H), 7.58 (d, J = 7.6 Hz, 1H), 7.48 (d, J = |
| | | | | 8.4 Hz, 2H), 7.45-7.41 (m, 3H), 7.38 |
| | | | | (s, 2H), 7.37-7.32 (m, 5H), 7.31-7.26 |
| | | | | (m, 3H), 7.21-7.17 (m, 1H), 7.13 (dd, J = |
| | | | | 1.2, 7.6 Hz, 1H), 6.91 (d, J = 9.2 Hz, |
| | | | | 1H), 6.78-6.70 (m, 3H), 5.21 (d, J = 4.4 |
| | | | | Hz, 1H), 5.10 (s, 1H), 4.96-4.88 (m, |
| | | | | 1H), 4.51 (d, J = 9.2 Hz, 1H), 4.42 (t, J = |
| | | | | 8.0 Hz, 1H), 4.30 (td, J = 4.0, 8.0 Hz, |
| | | | | 2H), 4.06-3.98 (m, 1H), 3.76-3.70 (m, |
| | | | | 1H), 3.60 (d, J = 4.0 Hz, 2H), 3.45-3.38 |
| | | | | (m, 8H), 3.03-2.95 (m, 1H), 2.90-2.82 |
| | | | | (m, 2H), 2.57 (s, 4H), 2.45 (s, 3H), 2.36- |
| | | | | 2.32 (m, 4H), 2.25 (t, J = 7.2 Hz, 3H), |
| | | | | 2.14-2.06 (m, 2H), 2.04-1.97 (m, 2H), |
| | | | | 1.90 (d, J = 12.4 Hz, 1H), 1.79 (ddd, J = |
| | | | | 4.4, 8.4, 12.8 Hz, 1H), 1.72-1.65 (m, |
| | | | | 2H), 1.61 (dd, J = 4.0, 6.8 Hz, 1H), 1.53- |
| | | | | 1.42 (m, 5H), 1.37 (d, J = 6.8 Hz, 3H), |
| | | | | 1.28-1.22 (m, 3H), 1.13-1.08 (m, 1H), |
| | | | | 0.98 (br d, J = 0.8 Hz, 2H), 0.93 (s, 9H), |
| | | | | 0.84 (dd, J = 3.2, 11.6 Hz, 1H) |
| I- | A | NL | 1617.5 | 10.10 (s, 1H), 8.98 (s, 1H), 8.39 (d, J = |
| 123 | | | | 8.0 Hz, 1H), 8.19 (s, 1H), 8.07 (s, 1H), |
| | | | | 7.96-7.89 (m, 2H), 7.82 (d, J = 9.2 Hz, |
| | | | | 1H), 7.64 (dd, J = 8.8, 18.4 Hz, 4H), 7.53 |
| | | | | (d, J = 8.4 Hz, 2H), 7.45-7.42 (m, 2H), |
| | | | | 7.40-7.33 (m, 7H), 7.32-7.25 (m, 4H), |
| | | | | 7.21-7.17 (m, 1H), 6.94 (d, J = 9.2 Hz, |
| | | | | 2H), 6.90-6.85 (m, 1H), 6.72 (d, J = 8.8 |
| | | | | Hz, 2H), 5.37 (d, J = 4.0 Hz, 1H), 5.12 |
| | | | | (d, J = 3.2 Hz, 1H), 4.96-4.89 (m, 1H), |
| | | | | 4.52 (d, J = 9.2 Hz, 1H), 4.44 (t, J = 8.0 |
| | | | | Hz, 1H), 4.40-4.36 (m, 1H), 4.31-4.26 |
| | | | | (m, 1H), 4.05-3.95 (m, 1H), 3.69 (d, J = |
| | | | | 12.4 Hz, 2H), 3.64-3.50 (m, 6H), 2.63 |
| | | | | (d, J = 12.0 Hz, 3H), 2.54 (s, 2H), 2.47- |
| | | | | 2.44 (m, 6H), 2.06-1.89 (m, 5H), 1.84- |
| | | | | 1.75 (m, 3H), 1.74-1.58 (m, 6H), 1.54- |
| | | | | 1.43 (m, 1H), 1.38 (d, J = 7.2 Hz, 3H), |
| | | | | 1.26-1.21 (m, 1H), 1.17-1.11 (m, 1H), |
| | | | | 1.06-0.98 (m, 2H), 0.97-0.93 (m, 9H), |
| | | | | 0.88-0.82 (m, 1H) |
| I- | QM | KT | 1536.5 | 9.00-8.97 (m, 1H), 8.37 (d, J = 7.6 Hz, |
| 125 | | | | 1H), 8.13-8.03 (m, 1H), 8.00-7.92 (m, |
| | | | | 1H), 7.79 (d, J = 9.2 Hz, 1H), 7.69-7.64 |
| | | | | (m, 2H), 7.58 (d, J = 7.2 Hz, 1H), 7.48 |
| | | | | (d, J = 8.4 Hz, 2H), 7.45-7.40 (m, 3H), |
| | | | | 7.40-7.32 (m, 7H), 7.31-7.26 (m, 3H), |
| | | | | 7.22-7.17 (m, 1H), 7.13 (dd, J = 1.2, 7.6 |
| | | | | Hz, 1H), 6.89-6.84 (m, 1H), 6.73 (d, J = |
| | | | | 8.8 Hz, 2H), 5.22 (d, J = 4.2 Hz, 1H), |
| | | | | 5.10 (d, J = 3.6 Hz, 1H), 4.96-4.88 (m, |
| | | | | 1H), 4.51 (d, J = 9.6 Hz, 1H), 4.41 (t, J = |
| | | | | 8.0 Hz, 1H), 4.33-4.27 (m, 2H), 4.18 (s, |
| | | | | 2H), 4.01-3.88 (m, 4H), 3.83-3.70 (m, |
| | | | | 2H), 3.62-3.54 (m, 3H), 3.31-3.19 (m, |
| | | | | 4H), 2.61-2.54 (m, 1H), 2.45 (s, 3H), |
| | | | | 2.44-2.39 (m, 1H), 2.22 (dd, J = 7.2, |
| | | | | 14.3 Hz, 1H), 2.12-2.07 (m, 1H), 2.02- |
| | | | | 1.93 (m, 3H), 1.89 (br d, J = 13.2 Hz, |
| | | | | 1H), 1.79 (ddd, J = 4.8, 8.4, 12.8 Hz, |
| | | | | 3H), 1.65-1.57 (m, 1H), 1.56-1.39 (m, |
| | | | | 5H), 1.37 (d, J = 7.2 Hz, 3H), 1.24-1.19 |
| | | | | (m, 2H), 1.17-0.96 (m, 3H), 0.95-0.91 |
| | | | | (m, 9H), 0.90-0.75 (m, 2H) |
| I- | QP | MK | 1578.5 | 9.03-8.95 (m, 1H), 8.40-8.33 (m, 1H), |
| 126 | | | | 8.13-8.04 (m, 1H), 8.01-7.88 (m, 1H), |
| | | | | 7.84-7.75 (m, 1H), 7.66 (d, J = 8.4 Hz, |
| | | | | 2H), 7.58 (d, J = 7.8 Hz, 1H), 7.50-7.47 |
| | | | | (m, 2H), 7.45-7.41 (m, 3H), 7.41-7.37 |
| | | | | (m, 2H), 7.36-7.33 (m, 5H), 7.32-7.27 |
| | | | | (m, 3H), 7.23-7.19 (m, 1H), 7.15-7.12 |
| | | | | (m, 1H), 6.92-6.83 (m, 1H), 6.76-6.66 |
| | | | | (m, 3H), 5.22-5.19 (m, 1H), 5.09 (d, J = |
| | | | | 3.6 Hz, 1H), 4.91 (br t, J = 7.2 Hz, 1H), |
| | | | | 4.53-4.48 (m, 1H), 4.44-4.38 (m, 1H), |
| | | | | 4.33-4.26 (m, 2H), 4.04-3.95 (m, 1H), |
| | | | | 3.76-3.67 (m, 1H), 3.64-3.53 (m, 3H), |
| | | | | 3.20-2.78 (m, 6H), 2.45 (s, 3H), 2.30- |
| | | | | 2.21 (m, 4H), 2.17-1.97 (m, 5H), 1.94- |
| | | | | 1.85 (m, 2H), 1.84-1.76 (m, 2H), 1.64- |
| | | | | 1.42 (m, 10H), 1.37 (br d, J = 7.2 Hz, |
| | | | | 4H), 1.33-1.21 (m, 3H), 0.98 (br s, 4H), |
| | | | | 0.93 (s, 9H), 0.91-0.76 (m, 2H) |
| I- | NV | MK | 1622.6 | 11.73-11.58 (m, 1H), 9.02-8.94 (m, |
| 131 | | | | 1H), 8.42-8.31 (m, 1H), 8.19-8.08 (m, |
| | | | | 1H), 8.08-7.97 (m, 1H), 7.95-7.85 (m, |
| | | | | 1H), 7.82-7.75 (m, 1H), 7.54-7.45 (m, |
| | | | | 3H), 7.44-7.41 (m, 2H), 7.40-7.30 (m, |
| | | | | 5H), 7.08 (d, J = 8.0 Hz, 2H), 7.05-6.91 |
| | | | | (m, 1H), 6.76-6.61 (m, 1H), 6.52-6.34 |
| | | | | (m, 1H), 6.25-6.10 (m, 1H), 5.12-5.05 |
| | | | | (m, 1H), 4.95-4.88 (m, 1H), 4.54-4.48 |
| | | | | (m, 1H), 4.45-4.38 (m, 1H), 4.31 (s, |
| | | | | 1H), 3.65-3.55 (m, 6H), 3.43-3.38 (m, |
| | | | | 4H), 3.09-2.98 (m, 4H), 2.70 (s, 1H), |
| | | | | 2.45 (s, 6H), 2.41 (s, 3H), 2.30-2.22 (m, |
| | | | | 5H), 2.21-2.10 (m, 9H), 2.04-1.96 (m, |
| | | | | 2H), 1.89-1.70 (m, 5H), 1.53-1.41 (m, |
| | | | | 6H), 1.40-1.32 (m, 4H), 1.28-1.21 (m, |
| | | | | 2H), 0.93 (s, 9H), 0.90 (s, 3H) |
| I- | LB | RB | 1537.0 | 11.62-11.49 (m, 1H), 8.98 (s, 1H), 8.56 |
| 133 | | | | (t, J = 5.9 Hz, 1H), 7.99 (s, 1H), 7.93 (d, |
| | | | | J = 2.4 Hz, 1H), 7.88-7.82 (m, 1H), 7.73 |
| | | | | (d, J = 9.4 Hz, 1H), 7.55 (d, J = 8.6 Hz, |
| | | | | 1H), 8.21-7.49 (m, 1H), 7.47-7.31 (m, |
| | | | | 8H), 7.30 (s, 1H), 7.05 (d, J = 8.4 Hz, |
| | | | | 3H), 6.79-6.72 (m, 1H), 6.65-6.59 (m, |
| | | | | 1H), 6.34-6.23 (m, 2H), 5.13 (d, J = 3.4 |
| | | | | Hz, 1H), 4.58-4.49 (m, 1H), 4.47-4.38 |
| | | | | (m, 2H), 4.35 (s, 1H), 4.21 (dd, J = 5.3, |
| | | | | 15.9 Hz, 1H), 3.72-3.60 (m, 2H), 3.43 |
| | | | | (d, J = 1.6 Hz, 4H), 3.01 (s, 4H), 2.72 (s, |
| | | | | 2H), 2.44 (s, 3H), 2.33 (d, J = 4.6 Hz, |
| | | | | 4H), 2.28-2.23 (m, 4H), 2.19 (s, 4H), |
| | | | | 2.14 (d, J = 6.4 Hz, 2H), 2.13-2.07 (m, |
| | | | | 2H), 2.06-2.00 (m, 2H), 1.95 (s, 2H), |
| | | | | 1.90 (dt, J = 4.1, 8.7 Hz, 2H), 1.73-1.66 |
| | | | | (m, 2H), 1.47 (s, 4H), 1.38 (t, J = 6.2 Hz, |
| | | | | 2H), 1.24 (d, J = 8.1 Hz, 2H), 0.93 (d, |
| | | | | J = 2.4 Hz, 15H) |
| I- | NV | RC | 820.2 | 11.64 (s, 1H), 8.98 (s, 1H), 8.57 (t, J = |
| 134^{b} | | | | 6.0 Hz, 1H), 8.08 (s, 1H), 7.99 (s, 1H), |
| | | | | 7.86 (d, J = 9.2 Hz, 2H), 7.51 (d, J = 8.8 |
| | | | | Hz, 1H), 7.47 (d, J = 2.8 Hz, 1H), 7.45- |
| | | | | 7.33 (m, 8H), 7.22 (s, 1H), 7.08 (d, J = |
| | | | | 8.4 Hz, 2H), 6.91 (s, 1H), 6.65 (d, J = 9.2 |
| | | | | Hz, 1H), 6.36 (s, 1H), 6.21 (s, 1H), 5.13 |
| | | | | (d, J = 3.6 Hz, 1H), 4.55 (d, J = 9.2 Hz, |
| | | | | 1H), 4.47-4.41 (m, 2H), 4.35 (s, 1H), |
| | | | | 4.24-4.18 (m, 1H), 3.74 (s, 5H), 3.66 (s, |
| | | | | 3H), 3.62-3.55 (m, 8H), 3.44-3.40 (m, |
| | | | | 8H), 3.02 (s, 4H), 2.68 (s, 4H), 2.53- |
| | | | | 2.52 (m, 2H), 2.45-2.44 (m, 3H), 2.39 |
| | | | | (s, 4H), 2.26-2.22 (m, 4H), 2.18 (s, 4H), |
| | | | | 2.03 (s, 1H), 1.92 (dd, J = 4.4, 8.8 Hz, |
| | | | | 1H), 1.83 (s, 1H), 1.74-1.69 (m, 2H), |
| | | | | 1.45 (s, 3H), 1.24 (s, 2H), 0.94 (s, 9H), |
| | | | | 0.89 (s, 3H) |
| I- | QD | KT | 1541.6 | 8.98 (s, 1H), 8.37 (d, J = 8.0 Hz, 1H), |
| 136 | | | | 8.14-8.06 (m, 1H), 7.91 (br dd, J = 1.6, |
| | | | | 11.3 Hz, 1H), 7.79 (d, J = 9.2 Hz, 1H), |
| | | | | 7.48 (br d, J = 8.4 Hz, 1H), 7.46-7.41 |
| | | | | (m, 3H), 7.39-7.34 (m, 4H), 7.06 (d, J = |
| | | | | 8.4 Hz, 2H), 6.86 (br s, 4H), 6.63 (br dd, |
| | | | | J = 2.8, 8.8 Hz, 1H), 6.17 (d, J = 1.6 Hz, |
| | | | | 1H), 5.09 (d, J = 3.6 Hz, 1H), 4.95-4.89 |
| | | | | (m, 1H), 4.51 (d, J = 9.2 Hz, 1H), 4.42 (t, |
| | | | | J = 8.0 Hz, 1H), 4.30-4.25 (m, 1H), 3.72 |
| | | | | (s, 3H), 3.60 (br s, 1H), 3.43 (br d, J = 5.6 |
| | | | | Hz, 4H), 3.08-3.02 (m, 4H), 2.74 (br s, |
| | | | | 2H), 2.60 (br d, J = 1.6 Hz, 4H), 2.45 (s, |
| | | | | 3H), 2.39-2.35 (m, 4H), 2.28-2.23 (m, |
| | | | | 4H), 2.22-2.15 (m, 8H), 1.97 (br s, 2H), |
| | | | | 1.82-1.70 (m, 4H), 1.51-1.44 (m, 5H), |
| | | | | 1.39 (br s, 1H), 1.37 (d, J = 7.2 Hz, 3H), |
| | | | | 1.28-1.21 (m, 3H), 0.93 (d, J = 1.6 Hz, |
| | | | | 15H) |
| I- | NV | RJ | 1593.6 | 11.67-11.56 (m, 1H), 8.39-8.29 (m, |
| 139^{c} | | | | 1H), 8.12-8.02 (m, 1H), 8.01-7.93 (m, |
| | | | | 1H), 7.81-7.78 (m, 1H), 7.53-7.46 (m, |
| | | | | 2H), 7.41 (d, J = 8.4 Hz, 3H), 7.37-7.25 |
| | | | | (m, 5H), 7.24 (br s, 1H), 7.08 (d, J = 8.4 |
| | | | | Hz, 2H), 6.95-6.83 (m, 1H), 6.69-6.61 |
| | | | | (m, 1H), 6.39-6.32 (m, 1H), 6.23-6.17 |
| | | | | (m, 1H), 5.13-5.08 (m, 1H), 4.87-4.79 |
| | | | | (m, 1H), 4.76-4.71 (m, 1H), 4.54-4.48 |
| | | | | (m, 1H), 4.47-4.40 (m, 1H), 4.30-4.22 |
| | | | | (m, 1H), 4.14-4.10 (m, 1H), 3.62-3.54 |
| | | | | (m, 8H), 3.43-3.39 (m, 4H), 3.05-2.99 |
| | | | | (m, 4H), 2.70-2.65 (m, 2H), 2.38 (s, |
| | | | | 8H), 2.27-2.22 (m, 4H), 2.21-2.14 (m, |
| | | | | 8H), 2.13-2.07 (m, 2H), 2.04-1.95 (m, |
| | | | | 2H), 1.85 (d, J = 17.6 Hz, 3H), 1.73- |
| | | | | 1.68 (m, 2H), 1.54-1.37 (m, 7H), 1.29- |
| | | | | 1.20 (m, 5H), 0.92 (s, 9H), 0.89 (s, 3H) |
| I- | UR | KT | 1551.6 | 0.92 (s, 6 H) 0.94 (s, 9 H) 1.25 (d, J = 7.6 |
| 140 | | | | Hz, 2 H) 1.37 (s, 2 H) 1.39 (s, 3 H) 1.44- |
| | | | | 1.52 (m, 4 H) 1.71-1.78 (m, 2 H) 1.79- |
| | | | | 1.84 (m, 1 H) 1.95 (s, 2 H) 2.04 (s, 4 H) |
| | | | | 2.08-2.16 (m, 4 H) 2.20-2.31 (m, 4 H) |
| | | | | 2.40 (d, J = 13.2 Hz, 4 H) 2.46 (s, 3 H) |
| | | | | 2.55 (s, 1 H) 2.69 (s, 2 H) 3.25 (s, 4 H) |
| | | | | 3.43 (d, J = 5.2 Hz, 6 H) 3.61 (s, 2 H) 4.29 |
| | | | | (s, 1 H) 4.43 (t, J = 8.4 Hz, 1 H) 4.52 (d, |
| | | | | J = 9.2 Hz, 1 H) 4.92 (t, J = 7.2 Hz, 1 H) |
| | | | | 5.10 (d, J = 3.6 Hz, 1 H) 6.43-6.50 (m, 2 |
| | | | | H) 7.02 (d, J = 8.4 Hz, 2 H) 7.19 (d, J = 9.2 |
| | | | | Hz, 1 H) 7.34 (d, J = 8.4 Hz, 2 H) 7.37- |
| | | | | 7.40 (m, 2 H) 7.43 (s, 2 H) 7.45 (s, 1 H) |
| | | | | 7.51 (t, J = 2.8 Hz, 1 H) 7.75 (s, 1 H) |
| | | | | 7.78-7.86 (m, 2 H) 8.04-8.13 (m, 2 H) 8.21 |
| | | | | (s, 1 H) 8.37 (d, J = 7.6 Hz, 1 H) 8.99 (s, |
| | | | | 1 H) 10.81-11.34 (m, 1 H) 11.67 (s, 1 H) |
| I- | RK | KT | 1565.6 | 11.63-11.56 (m, 1H), 9.04-8.94 (m, 1H), |
| 141 | | | | 8.36 (d, J = 8.0 Hz, 1H), 8.09-8.02 |
| | | | | (m, 1H), 7.98 (s, 1H), 7.85 (d, J = 8.4 Hz, |
| | | | | 1H), 7.78 (d, J = 9.2 Hz, 2H), 7.55 (d, |
| | | | | J = 8.4 Hz, 1H), 7.49-7.41 (m, 4H), 7.41- |
| | | | | 7.37 (m, 3H), 7.35 (d, J = 8.0 Hz, 2H), |
| | | | | 7.20-7.08 (m, 1H), 7.05 (d, J = 8.4 Hz, |
| | | | | 2H), 6.93-6.85 (m, 1H), 6.64 (d, J = 8.0 |
| | | | | Hz, 1H), 6.38-6.33 (m, 1H), 6.23 (s, |
| | | | | 1H), 5.15-5.06 (m, 1H), 4.96-4.89 (m, |
| | | | | 1H), 4.52 (d, J = 9.6 Hz, 1H), 4.43 (t, J = |
| | | | | 8.0 Hz, 1H), 4.29 (s, 1H), 3.60 (s, 3H), |
| | | | | 3.40-3.36 (m, 2H), 3.03 (s, 4H), 2.74 (s, |
| | | | | 3H), 2.46 (s, 3H), 2.29-2.21 (m, 2H), |
| | | | | 2.20 (s, 5H), 2.17-2.06 (m, 4H), 2.02 (d, |
| | | | | J = 7.2 Hz, 3H), 1.96 (s, 2H), 1.80 (d, J = |
| | | | | 4.0 Hz, 6H), 1.47 (d, J = 7.2 Hz, 6H), |
| | | | | 1.42-1.36 (m, 6H), 1.30-1.16 (m, 3H), |
| | | | | 0.94 (s, 15H) |
| I- | KS | RB | 1510.5 | 8.98 (s, 1H), 8.60-8.52 (m, 1H), 8.14 (d, |
| 143 | | | | J = 2.0 Hz, 1H), 7.96 (dd, J = 2.0, 9.2 Hz, |
| | | | | 1H), 7.85 (d, J = 9.2 Hz, 1H), 7.67 (d, J = |
| | | | | 8.8 Hz, 2H), 7.58 (d, J = 7.2 Hz, 1H), |
| | | | | 7.48 (d, J = 8.4 Hz, 2H), 7.45-7.39 (m, |
| | | | | 5H), 7.38-7.35 (m, 2H), 7.34-7.29 (m, |
| | | | | 4H), 7.26 (t, J = 7.6 Hz, 2H), 7.19-7.12 |
| | | | | (m, 2H), 7.04 (d, J = 10.0 Hz, 1H), 6.95 |
| | | | | (d, J = 5.2 Hz, 1H), 6.80 (d, J = 9.2 Hz, |
| | | | | 2H), 5.23 (d, J = 4.4 Hz, 1H), 5.12 (d, J = |
| | | | | 3.6 Hz, 1H), 4.54 (d, J = 9.2 Hz, 1H), |
| | | | | 4.48-4.40 (m, 2H), 4.35 (s, 1H), 4.30 |
| | | | | (dd, J = 4.0, 7.8 Hz, 1H), 4.22 (dd, J = |
| | | | | 5.6, 15.9 Hz, 1H), 4.14-4.06 (m, 1H), |
| | | | | 3.88-3.80 (m, 1H), 3.71-3.62 (m, 3H), |
| | | | | 3.51-3.43 (m, 1H), 3.41-3.35 (m, 3H), |
| | | | | 3.28 (s, 2H), 2.71-2.63 (m, 2H), 2.45 (s, |
| | | | | 3H), 2.29-2.20 (m, 4H), 2.16-2.05 (m, |
| | | | | 2H), 2.05-1.96 (m, 2H), 1.94-1.87 (m, |
| | | | | 2H), 1.85-1.74 (m, 1H), 1.73-1.61 (m, |
| | | | | 1H), 1.55-1.39 (m, 5H), 1.31-1.17 (m, |
| | | | | 3H), 1.15-0.98 (m, 3H), 0.96-0.91 (m, |
| | | | | 9H), 0.89-0.73 (m, 2H) |
| I- | QI | RW | 1508.6 | 11.69-11.60 (m, 1H), 8.35 (d, J = 8.0 |
| 144 | | | | Hz, 1H), 8.10 (br s, 1H), 8.01 (s, 1H), |
| | | | | 7.89 (br d, J = 8.9 Hz, 1H), 7.84-7.78 |
| | | | | (m, 1H), 7.54-7.45 (m, 3H), 7.41 (d, J = |
| | | | | 8.1 Hz, 2H), 7.33 (dd, J = 8.3, 15.1 Hz, |
| | | | | 5H), 7.05 (d, J = 8.4 Hz, 2H), 6.99-6.91 |
| | | | | (m, 1H), 6.69-6.63 (m, 1H), 6.38 (br s, |
| | | | | 1H), 6.20 (s, 1H), 5.10 (d, J = 3.5 Hz, |
| | | | | 1H), 4.87-4.79 (m, 1H), 4.74 (t, J = 5.9 |
| | | | | Hz, 1H), 4.54-4.49 (m, 1H), 4.44 (t, J = |
| | | | | 8.0 Hz, 1H), 4.27 (br d, J = 2.3 Hz, 1H), |
| | | | | 4.14 (s, 1H), 3.61 (br s, 2H), 3.59-3.52 |
| | | | | (m, 3H), 3.50-3.42 (m, 3H), 3.37 (br s, |
| | | | | 2H), 3.05 (br s, 4H), 2.74 (br s, 3H), 2.31- |
| | | | | 2.22 (m, 4H), 2.21-2.08 (m, 8H), 2.05- |
| | | | | 1.93 (m, 4H), 1.91-1.67 (m, 6H), 1.57- |
| | | | | 1.43 (m, 5H), 1.39 (br t, J = 6.4 Hz, 2H), |
| | | | | 1.25 (br d, J = 3.3 Hz, 3H), 0.93 (s, 15H) |
| I- | QI | LG | 1579.6 | 11.64 (d, J = 2.0 Hz, 1H), 8.98 (s, 1H), |
| 145 | | | | 8.37 (d, J = 7.6 Hz, 1H), 8.10 (s, 1H), |
| | | | | 8.00 (s, 1H), 7.92-7.84 (m, 1H), 7.78 (d, |
| | | | | J = 9.6 Hz, 1H), 7.45-7.42 (m, 2H), 7.49 |
| | | | | (d, J = 3.2 Hz, 2H), 7.40-7.32 (m, 5H), |
| | | | | 7.05 (d, J = 8.4 Hz, 2H), 6.95 (dd, J = 2.4, |
| | | | | 9.2 Hz, 1H), 6.69-6.63 (m, 1H), 6.38 (s, |
| | | | | 1H), 6.20 (s, 1H), 5.09 (d, J = 3.6 Hz, |
| | | | | 1H), 4.96-4.88 (m, 1H), 4.52 (d, J = 9.2 |
| | | | | Hz, 1H), 4.42 (t, J = 8.0 Hz, 1H), 4.28 (d, |
| | | | | J = 3.2 Hz, 1H), 3.60 (s, 2H), 3.53-3.42 |
| | | | | (m, 4H), 3.36 (d, J = 5.2 Hz, 2H), 3.04 (s, |
| | | | | 4H), 2.79-2.70 (m, 4H), 2.46 (s, 3H), |
| | | | | 2.29-2.24 (m, 3H), 2.23-2.08 (m, 9H), |
| | | | | 2.07-1.97 (m, 2H), 1.96 (s, 2H), 1.87 (s, |
| | | | | 1H), 1.81-1.69 (m, 4H), 1.47 (d, J = 6.4 |
| | | | | Hz, 5H), 1.40-1.36 (m, 5H), 1.25 (d, J = |
| | | | | 3.2 Hz, 5H), 0.93 (d, J = 3.2 Hz, 15H) |
| I- | LB | RX | 1480.5 | 11.59 (s, 1H), 8.99 (s, 1H), 8.37 (d, J = |
| 146^{c} | | | | 8.0 Hz, 1H), 8.04 (s, 1H), 7.97 (d, J = 2.4 |
| | | | | Hz, 1H), 7.84-7.77 (m, 2H), 7.54 (d, J = |
| | | | | 8.8 Hz, 1H), 7.45 (d, J = 4.0 Hz, 2H), |
| | | | | 7.43 (s, 1H), 7.39 (s, 1H), 7.37 (d, J = 4.8 |
| | | | | Hz, 3H), 7.34 (s, 1H), 7.05 (d, J = 8.4 Hz, |
| | | | | 3H), 6.85 (d, J = 8.0 Hz, 1H), 6.64 (d, |
| | | | | J = 8.8 Hz, 1H), 6.35 (s, 1H), 6.23 (s, 1H), |
| | | | | 5.16-5.06 (m, 1H), 4.96-4.89 (m, 1H), |
| | | | | 4.53 (d, J = 9.6 Hz, 1H), 4.42 (t, J = 8.0 |
| | | | | Hz, 1H), 4.29 (s, 1H), 3.67-3.54 (m, |
| | | | | 3H), 3.02 (s, 6H), 2.73 (s, 3H), 2.46 (s, |
| | | | | 5H), 2.30-2.23 (m, 2H), 2.23-2.12 (m, |
| | | | | 9H), 2.12-2.06 (m, 1H), 2.00 (d, J = 9.6 |
| | | | | Hz, 2H), 1.96 (s, 2H), 1.80 (dt, J = 4.4, |
| | | | | 8.3 Hz, 1H), 1.72 (s, 2H), 1.53-1.44 (m, |
| | | | | 5H), 1.41-1.36 (m, 6H), 1.26 (s, 8H), |
| | | | | 0.94 (d, J = 3.2 Hz, 15H) |
| I- | RY | RZ | 1508.6 | 11.68 (s, 1H), 8.37-8.32 (m, 1H), 8.16- |
| 147 | | | | 8.09 (m, 1H), 8.03 (d, J = 2.4 Hz, 1H), |
| | | | | 7.90 (d, J = 8.0 Hz, 1H), 7.80 (d, J = 9.2 |
| | | | | Hz, 1H), 7.52-7.46 (m, 3H), 7.42-7.39 |
| | | | | (m, 2H), 7.32 (dd, J = 8.4, 14.4 Hz, 5H), |
| | | | | 7.05-6.99 (m, 3H), 6.70-6.63 (m, 1H), |
| | | | | 6.42-6.37 (m, 1H), 6.18 (d, J = 1.6 Hz, |
| | | | | 1H), 5.11-5.08 (m, 1H), 4.86-4.80 (m, |
| | | | | 1H), 4.73 (t, J = 6.0 Hz, 1H), 4.51 (d, J = |
| | | | | 9.2 Hz, 1H), 4.44 (t, J = 8.0 Hz, 1H), 4.26 |
| | | | | (d, J = 1.6 Hz, 1H), 4.13-4.11 (m, 1H), |
| | | | | 3.60 (s, 2H), 3.55 (d, J = 6.4 Hz, 2H), |
| | | | | 3.44 (s, 4H), 3.06 (s, 4H), 2.74 (s, 3H), |
| | | | | 2.44-2.32 (m, 6H), 2.26 (t, J = 7.2 Hz, |
| | | | | 4H), 2.22-2.04 (m, 9H), 2.02-1.97 (m, |
| | | | | 1H), 1.95 (s, 2H), 1.80-1.71 (m, 3H), |
| | | | | 1.49-1.35 (m, 7H), 1.24 (d, J = 2.8 Hz, |
| | | | | 4H), 0.92 (s, 15H) |
| I- | SE | RW | 1436.5 | 0.93 (s, 15 H) 1.22-1.29 (m, 2 H) 1.39 |
| 148 | | | | (t, J = 6.4 Hz, 2 H) 1.45-1.54 (m, 4 H) |
| | | | | 1.75-1.93 (m, 2 H) 1.93-2.04 (m, 4 H) |
| | | | | 2.14 (d, J = 6.0 Hz, 4 H) 2.18-2.30 (m, 8 |
| | | | | H) 2.78 (s, 1 H) 3.03-3.13 (m, 4 H) 3.36- |
| | | | | 3.42 (m, 2 H) 3.47-3.53 (m, 1 H) 3.54- |
| | | | | 3.59 (m, 2 H) 3.61 (s, 2 H) 3.85 (dd, |
| | | | | J = 10.4, 6.32 Hz, 1 H) 4.13 (s, 1 H) 4.24- |
| | | | | 4.40 (m, 2 H) 4.45 (t, J = 8.0 Hz, 1 H) |
| | | | | 4.51 (dd, J = 9.2, 2.56 Hz, 1 H) 4.74 (t, |
| | | | | J = 5.6 Hz, 1 H) 4.79-4.88 (m, 1 H) 5.10 |
| | | | | (d, J = 2.4 Hz, 1 H) 6.20 (s, 1 H) 6.40 (s, |
| | | | | 1 H) 6.68 (d, J = 7.2 Hz, 1 H) 6.87 (s, 1 |
| | | | | H) 7.05 (m, J = 8.4 Hz, 2 H) 7.15 (s, 1 H) |
| | | | | 7.31 (d, J = 8.4 Hz, 2 H) 7.35 (d, J = 8.4 |
| | | | | Hz, 2 H) 7.41 (m, J = 8.4 Hz, 2 H) 7.46- |
| | | | | 7.56 (m, 3 H) 7.81 (dd, J = 9.2, 3.69 Hz, |
| | | | | 1 H) 7.99 (d, J = 8.8 Hz, 1 H) 8.04 (s, 1 |
| | | | | H) 8.18 (s, 1 H) 8.35 (d, J = 8.4 Hz, 1 H) |
| | | | | 11.69 (s, 1 H) |
| I- | SB | RW | 1451.2 | 11.68 (s, 1H), 11.59-11.26 (m, 1H), 8.35 |
| 149 | | | | (d, J = 8.0 Hz, 1H), 8.14 (d, J = 4.4 Hz, |
| | | | | 1H), 8.03 (d, J = 2.4 Hz, 1H), 7.95-7.88 |
| | | | | (m, 1H), 7.81 (d, J = 9.2 Hz, 1H), 7.50 |
| | | | | (d, J = 2.4 Hz, 2H), 7.48 (d, J = 8.8 Hz, |
| | | | | 1H), 7.41 (d, J = 8.0 Hz, 2H), 7.32 (dd, |
| | | | | J = 8.4, 15.2 Hz, 4H), 7.16-7.10 (m, 1H), |
| | | | | 7.04 (d, J = 8.4 Hz, 2H), 6.77-6.70 (m, |
| | | | | 1H), 6.67 (d, J = 9.2 Hz, 1H), 6.39 (dd, J = |
| | | | | 2.0, 3.2 Hz, 1H), 6.19 (s, 1H), 5.10 (d, |
| | | | | J = 3.6 Hz, 1H), 4.86-4.79 (m, 1H), 4.74 |
| | | | | (t, J = 6.0 Hz, 1H), 4.51 (d, J = 9.2 Hz, |
| | | | | 1H), 4.44 (t, J = 8.0 Hz, 1H), 4.28-4.21 |
| | | | | (m, 2H), 4.13 (s, 1H), 3.81 (d, J = 12.0 |
| | | | | Hz, 2H), 3.61 (s, 2H), 3.58-3.53 (m, |
| | | | | 2H), 3.20-3.14 (m, 2H), 3.06 (s, 4H), |
| | | | | 2.84-2.73 (m, 4H), 2.30 (d, J = 7.6 Hz, |
| | | | | 2H), 2.27-2.22 (m, 2H), 2.21 (s, 2H), |
| | | | | 2.16-2.13 (m, 2H), 2.13-2.06 (m, 2H), |
| | | | | 2.00 (d, J = 9.6 Hz, 1H), 1.95 (s, 2H), |
| | | | | 1.88 (d, J = 10.0 Hz, 2H), 1.80-1.74 (m, |
| | | | | 1H), 1.52-1.45 (m, 4H), 1.38 (t, J = 6.4 |
| | | | | Hz, 2H), 1.26 (d, J = 7.2 Hz, 2H), 0.92 |
| | | | | (d, J = 2.4 Hz, 15H) |
| I- | NF | MZ | 1584.6 | 9.09-8.93 (m, 1H), 8.40-8.32 (m, 2H), |
| 151 | | | | 8.07 (s, 1H), 7.93 (s, 1H), 7.76 (d, J = 8.8 |
| | | | | Hz, 1H), 7.68 (d, J = 8.8 Hz, 2H), 7.50- |
| | | | | 7.42 (m, 5H), 7.40-7.27 (m, 9H), 7.22- |
| | | | | 7.16 (m, 2H), 7.08 (d, J = 7.6 Hz, 1H), |
| | | | | 6.91-6.81 (m, 1H), 6.72 (d, J = 8.4 Hz, |
| | | | | 2H), 5.21 (d, J = 4.0 Hz, 1H), 5.09 (d, J = |
| | | | | 3.6 Hz, 1H), 4.92 (quin, J = 7.2 Hz, |
| | | | | 1H), 4.52 (d, J = 9.2 Hz, 1H), 4.46-4.40 |
| | | | | (m, 1H), 4.34-4.27 (m, 4H), 4.06-3.95 |
| | | | | (m, 1H), 3.74-3.51 (m, 7H), 2.46 (s, |
| | | | | 3H), 2.43-2.38 (m, 2H), 2.27-2.21 (m, |
| | | | | 2H), 2.17-2.10 (m, 4H), 2.04-1.97 (m, |
| | | | | 2H), 1.96-1.87 (m, 2H), 1.81 (dt, J = |
| | | | | 4.0, 8.5 Hz, 2H), 1.60-1.42 (m, 7H), |
| | | | | 1.38 (d, J = 7.2 Hz, 3H), 1.25 (s, 11H), |
| | | | | 1.19-1.07 (m, 2H), 1.05-0.99 (m, 1H), |
| | | | | 0.94 (s, 9H), 0.85 (d, J = 11.2 Hz, 1H) |
| I- | PF | KT | 773.8 | 11.54 (s, 1H), 8.96-9.01 (m, 1H), 8.33- |
| 152^{b} | | | | 8.41 (m, 1H), 7.95 (s, 1 H), 7.89-7.91 |
| | | | | (m, 1H), 7.77-7.82 (m, 1H), 7.66-7.72 |
| | | | | (m, 1H), 7.53-7.58 (m, 1H), 7.48-7.52 |
| | | | | (m, 1 H), 7.37-7.45 (m, 6H), 7.18-7.24 |
| | | | | (m, 2H), 7.08-7.16 (m, 2H), 6.89-6.95 |
| | | | | (m, 1H) 6.65-6.71 (m, 1 H) 6.58-6.64 |
| | | | | (m, 1H), 6.30 (s, 1H), 6.24 (s, 1H), 5.10 |
| | | | | (d, J = 3.63 Hz, 1H), 4.89-4.96 (m, 1H), |
| | | | | 4.49-4.54 (m, 1H), 4.40-4.46 (m, 1H), |
| | | | | 4.32 (s, 1H), 3.60 (s, 3H), 3.44 (s, 4H), |
| | | | | 2.93-2.99 (m, 2H), 2.86-2.90 (m, 2H), |
| | | | | 2.68 (s, 2H), 2.31-2.36 (m, 9H), 2.27 |
| | | | | (dd, J = 15.01, 7.38 Hz, 7H), 2.10-2.16 |
| | | | | (m, 2H), 1.96-2.06 (m, 2H), 1.65-1.77 |
| | | | | (m, 7H), 1.46 (dd, J = 8.19, 3.94 Hz, 5H), |
| | | | | 1.42-1.43 (m, 1H), 1.38 (d, J = 6.88 Hz, |
| | | | | 6H), 1.21-1.27 (m, 3H), 1.15-1.18 (m, |
| | | | | 6H), 0.94 (s, 9H) |
| I- | PH | KT | 1516.1 | 11.72-11.57 (m, 1H), 8.99 (s, 1H), 8.37 |
| 153 | | | | (d, J = 8.0 Hz, 1H), 8.29-7.94 (m, 2H), |
| | | | | 7.91-7.72 (m, 2H), 7.58-7.36 (m, 9H), |
| | | | | 7.30-7.03 (m, 4H), 6.70-6.62 (m, 1H), |
| | | | | 6.37 (s, 1H), 6.18 (s, 1H), 5.10 (d, J = 3.6 |
| | | | | Hz, 1H), 4.98-4.88 (m, 1H), 4.55-4.49 |
| | | | | (m, 1H), 4.46-4.35 (m, 2H), 4.32-4.25 |
| | | | | (m, 1H), 3.91-3.81 (m, 1H), 3.64-3.58 |
| | | | | (m, 2H), 3.26-3.19 (m, 2H), 3.26-3.19 |
| | | | | (m, 1H), 3.06-2.89 (m, 6H), 2.46 (s, |
| | | | | 3H), 2.30-2.20 (m, 4H), 2.17-2.07 (m, |
| | | | | 2H), 2.05-1.96 (m, 2H), 1.89-1.76 (m, |
| | | | | 4H), 1.71-1.62 (m, 3H), 1.56-1.31 (m, |
| | | | | 16H), 1.28-1.09 (m, 11H), 1.07-0.96 |
| | | | | (m, 2H), 0.94 (s, 9H) |
| I- | A | PX | 1523.6 | 11.80 (s, 1H), 11.74-11.70 (m, 1H), |
| 155 | | | | 11.07-10.98 (m, 1H), 10.66-10.59 (m, |
| | | | | 1H), 10.57-10.51 (m, 1H), 10.20-10.09 |
| | | | | (m, 1H), 9.03-9.00 (m, 1H), 8.41-8.37 |
| | | | | (m, 1H), 8.28-8.19 (m, 2H), 8.07 (d, J = |
| | | | | 2.4 Hz, 1H), 7.99-7.94 (m, 1H), 7.80 (d, |
| | | | | J = 9.2 Hz, 1H), 7.61 (d, J = 2.4 Hz, 1H), |
| | | | | 7.55 (t, J = 2.8 Hz, 1H), 7.51 (d, J = 8.8 |
| | | | | Hz, 1H), 7.45-7.43 (m, 3H), 7.40-7.38 |
| | | | | (m, 4H), 7.19-7.14 (m, 1H), 7.10 (d, J = |
| | | | | 8.4 Hz, 2H), 6.74-6.71 (m, 1H), 6.44 (d, |
| | | | | J = 1.2 Hz, 1H), 6.25 (s, 1H), 4.92 (t, J = |
| | | | | 7.2 Hz, 1H), 4.51 (d, J = 9.2 Hz, 1H), |
| | | | | 4.42 (t, J = 8.0 Hz, 1H), 4.29 (s, 1H), |
| | | | | 4.23-4.17 (m, 2H), 4.14-4.05 (m, 2H), 3.63 |
| | | | | (d, J = 4.0 Hz, 2H), 3.61 (d, J = 2.8 Hz, |
| | | | | 2H), 3.53 (s, 3H), 3.46 (d, J = 6.0 Hz, |
| | | | | 3H), 3.26 (d, J = 8.4 Hz, 4H), 3.16-3.06 |
| | | | | (m, 4H), 2.69 (d, J = 12.0 Hz, 2H), 2.46 |
| | | | | (s, 2H), 2.37-2.31 (m, 3H), 2.24 (d, J = |
| | | | | 6.4 Hz, 1H), 2.16-2.11 (m, 1H), 2.01 (s, |
| | | | | 3H), 1.83-1.71 (m, 4H), 1.44 (t, J = 6.4 |
| | | | | Hz, 4H), 1.38 (d, J = 6.8 Hz, 4H), 1.35- |
| | | | | 1.28 (m, 1H), 1.28-1.19 (m, 1H), 1.18- |
| | | | | 1.06 (m, 1H), 0.94 (s, 12H) |
| I- | KS | QR | 1588.5 | 8.98 (s, 1H), 8.37 (d, J = 7.6 Hz, 1H), |
| 159^{d} | | | | 8.13 (s, 1H), 7.95 (d, J = 8.9 Hz, 1H), |
| | | | | 7.78-7.71 (m, 1H), 7.69-7.65 (m, 2H), |
| | | | | 7.58 (d, J = 7.6 Hz, 1H), 7.49-7.41 (m, |
| | | | | 5H), 7.40-7.30 (m, 8H), 7.28-7.24 (m, |
| | | | | 2H), 7.19-7.12 (m, 2H), 7.06-7.00 (m, |
| | | | | 1H), 6.98-6.90 (m, 1H), 6.79 (br d, J = |
| | | | | 8.6 Hz, 2H), 5.22 (d, J = 4.1 Hz, 1H), |
| | | | | 5.08 (br d, J = 2.6 Hz, 1H), 4.96-4.88 |
| | | | | (m, 1H), 4.50 (br d, J = 9.1 Hz, 1H), 4.42 |
| | | | | (t, J = 8.1 Hz, 1H), 4.32-4.26 (m, 2H), |
| | | | | 4.13-4.05 (m, 1H), 3.86-3.79 (m, 1H), |
| | | | | 3.68-3.58 (m, 3H), 3.49-3.37 (m, 3H), |
| | | | | 3.29-3.26 (m, 1H), 2.71-2.60 (m, 2H), |
| | | | | 2.45 (s, 4H), 2.36-2.29 (m, 2H), 2.24- |
| | | | | 2.10 (m, 3H), 2.03-1.94 (m, 3H), 1.79 |
| | | | | (br s, 4H), 1.67-1.52 (m, 5H), 1.47- |
| | | | | 1.24 (m, 7H), 1.14-0.97 (m, 3H), 0.96- |
| | | | | 0.85 (m, 13H), 0.84-0.76 (m, 1H) |
| I- | QU | LG | 1636.6 | 11.65 (s, 1H), 8.98 (s, 1H), 8.37 (d, J = |
| 161 | | | | 7.6 Hz, 1H), 8.09 (s, 1H), 8.00 (d, J = 2.4 |
| | | | | Hz, 1H), 7.84 (d, J = 8.4 Hz, 1H), 7.81- |
| | | | | 7.76 (m, 1H), 7.53-7.47 (m, 2H), 7.47- |
| | | | | 7.41 (m, 3H), 7.40-7.35 (m, 4H), 7.31- |
| | | | | 7.20 (m, 1H), 7.14 (d, J = 8.4 Hz, 2H), |
| | | | | 6.98-6.86 (m, 1H), 6.72-6.60 (m, 1H), |
| | | | | 6.41-6.32 (m, 1H), 6.20 (s, 1H), 5.09 (d, |
| | | | | J = 3.6 Hz, 1H), 4.96-4.87 (m, 1H), 4.51 |
| | | | | (d, J = 9.2 Hz, 1H), 4.41 (t, J = 8.0 Hz, |
| | | | | 1H), 4.38-4.31 (m, 1H), 4.27 (s, 1H), |
| | | | | 3.88-3.72 (m, 1H), 3.59 (d, J = 3.6 Hz, |
| | | | | 6H), 3.38-3.34 (m, 4H), 3.08-3.00 (m, |
| | | | | 4H), 2.98-2.92 (m, 1H), 2.76 (s, 2H), |
| | | | | 2.52-2.51 (m, 2H), 2.45 (s, 3H), 2.39 (s, |
| | | | | 6H), 2.23 (dd, J = 7.2, 18.0 Hz, 10H), |
| | | | | 2.15-2.05 (m, 2H), 2.03-1.97 (m, 1H), |
| | | | | 1.92-1.78 (m, 2H), 1.75-1.67 (m, 4H), |
| | | | | 1.56-1.39 (m, 5H), 1.37 (d, J = 7.2 Hz, |
| | | | | 3H), 1.28-1.21 (m, 4H), 1.00 (dd, J = |
| | | | | 2.4, 12.8 Hz, 1H), 0.95-0.90 (m, 9H), |
| | | | | 0.90-0.82 (m, 1H) |
| I- | NV | QR | 840.0 | 11.75-11.58 (m, 1H), 9.04-8.93 (m, |
| 165^{b} | | | | 1H), 8.36 (d, J = 8.0 Hz, 1H), 8.15-8.08 |
| | | | | (m, 1H), 8.01 (d, J = 2.4 Hz, 1H), 7.88 |
| | | | | (d, J = 8.8 Hz, 1H), 7.81-7.70 (m, 1H), |
| | | | | 7.54-7.46 (m, 3H), 7.45-7.41 (m, 2H), |
| | | | | 7.40-7.34 (m, 3H), 7.33 (s, 1H), 7.32- |
| | | | | 7.27 (m, 1H), 7.08 (d, J = 8.4 Hz, 2H), |
| | | | | 7.02-6.93 (m, 1H), 6.66 (d, J = 9.6 Hz, |
| | | | | 1H), 6.44-6.34 (m, 1H), 6.24-6.14 (m, |
| | | | | 1H), 5.08 (d, J = 3.6 Hz, 1H), 4.96-4.87 |
| | | | | (m, 1H), 4.55-4.47 (m, 1H), 4.46-4.38 |
| | | | | (m, 1H), 4.32-4.24 (m, 1H), 3.64-3.57 |
| | | | | (m, 6H), 3.40 (d, J = 2.4 Hz, 6H), 3.04 (s, |
| | | | | 4H), 2.77-2.67 (m, 3H), 2.45 (s, 3H), |
| | | | | 2.41 (s, 7H), 2.27-2.10 (m, 11H), 2.07 |
| | | | | (s, 1H), 2.06-1.95 (m, 3H), 1.92-1.86 |
| | | | | (m, 1H), 1.85-1.76 (m, 2H), 1.75-1.70 |
| | | | | (m, 2H), 1.69-1.60 (m, 3H), 1.59-1.51 |
| | | | | (m, 2H), 1.46-1.29 (m, 7H), 1.00-0.84 |
| | | | | (m, 15H) |
| I- | LB | QR | 796.8 | 11.66 (s, 1H), 8.98 (s, 1H), 8.36 (d, J = |
| 166 ^{b} | | | | 8.4 Hz, 1H), 8.12 (s, 1H), 8.02 (d, J = 2.4 |
| | | | | Hz, 1H), 7.89 (d, J = 8.0 Hz, 1H), 7.79- |
| | | | | 7.70 (m, 1H), 7.52-7.45 (m, 3H), 7.45- |
| | | | | 7.41 (m, 2H), 7.40-7.36 (m, 2H), 7.34 |
| | | | | (d, J = 8.4 Hz, 3H), 7.04 (d, J = 8.4 Hz, |
| | | | | 2H), 7.01-6.94 (m, 1H), 6.71-6.61 (m, |
| | | | | 1H), 6.38 (d, J = 1.2 Hz, 1H), 6.19 (s, |
| | | | | 1H), 5.10-5.06 (m, 1H), 4.91 (t, J = 7.2 |
| | | | | Hz, 1H), 4.51 (dd, J = 3.6, 9.2 Hz, 1H), |
| | | | | 4.42 (t, J = 8.0 Hz, 1H), 4.27 (d, J = 2.4 |
| | | | | Hz, 1H), 3.61 (s, 2H), 3.45 (s, 4H), 3.37 |
| | | | | (s, 3H), 3.05 (s, 4H), 2.73 (s, 2H), 2.53- |
| | | | | 2.51 (m, 2H), 2.45 (s, 3H), 2.42-2.29 |
| | | | | (m, 6H), 2.25 (d, J = 6.4 Hz, 1H), 2.21- |
| | | | | 2.12 (m, 7H), 2.11-2.05 (m, 1H), 2.04- |
| | | | | 1.97 (m, 2H), 1.95 (s, 2H), 1.83-1.61 |
| | | | | (m, 6H), 1.60-1.54 (m, 1H), 1.41-1.34 |
| | | | | (m, 6H), 1.33-1.28 (m, 1H), 0.93 (d, J = |
| | | | | 4.8 Hz, 16H). |
| I- | RE | MK | 1712.7 | 11.61 (s, 1H), 8.98 (s, 1H), 8.37 (d, J = |
| 168^{d} | | | | 8.0 Hz, 1H), 8.06 (s, 1H), 7.98 (d, J = 2.0 |
| | | | | Hz, 1H), 7.85-7.75 (m, 2H), 7.52 (d, J = |
| | | | | 8.8 Hz, 1H), 7.49-7.27 (m, 9H), 7.17 (s, |
| | | | | 1H), 7.09 (d, J = 8.4 Hz, 2H), 6.87 (d, J = |
| | | | | 9.2 Hz, 1H), 6.64 (d, J = 7.4 Hz, 1H), |
| | | | | 6.35 (s, 1H), 6.21 (s, 1H), 5.10 (d, J = 3.6 |
| | | | | Hz, 1H), 4.91 (quin, J = 7.2 Hz, 1H), |
| | | | | 4.55-4.48 (m, 1H), 4.42 (t, J = 8.0 Hz, 1H), |
| | | | | 4.28 (d, J = 0.8 Hz, 1H), 3.64-3.50 (m, |
| | | | | 8H), 3.42-3.40 (m, 2H), 3.01 (s, 4H), |
| | | | | 2.69 (br s, 4H), 2.45 (s, 3H), 2.37 (d, J = |
| | | | | 5.2 Hz, 8H), 2.31-2.24 (m, 4H), 2.18 (s, |
| | | | | 6H), 2.14-2.09 (m, 2H), 2.07-1.98 (m, |
| | | | | 2H), 1.94-1.86 (m, 1H), 1.82-1.76 (m, |
| | | | | 1H), 1.73-1.67 (m, 2H), 1.61-1.42 (m, |
| | | | | 11H), 1.41-1.35 (m, 5H), 1.33 (b d, J = |
| | | | | 3.6 Hz, 2H), 1.27-1.22 (m, 1H), 0.93 (s, |
| | | | | 12H) |
| I- | RI | MK | 1662.6 | 11.61 (d, J = 1.6 Hz, 1H), 9.00-8.94 (m, |
| 170 | | | | 1H), 8.37 (d, J = 7.6 Hz, 1H), 8.08-8.03 |
| | | | | (m, 1H), 7.98 (s, 1H), 7.85-7.76 (m, 2H), |
| | | | | 7.53 (d, J = 8.8 Hz, 1H), 7.49-7.32 |
| | | | | (m, 9H), 7.22-7.12 (m, 1H), 7.08 (d, J = |
| | | | | 8.4 Hz, 2H), 6.92-6.79 (m, 1H), 6.65 (d, |
| | | | | J = 8.4 Hz, 1H), 6.38-6.33 (m, 1H), 6.25- |
| | | | | 6.18 (m, 1H), 5.10 (d, J = 2.8 Hz, 1H), |
| | | | | 4.97-4.88 (m, 1H), 4.52 (d, J = 9.3 Hz, |
| | | | | 1H), 4.43 (t, J = 8.0 Hz, 1H), 4.28 (s, |
| | | | | 1H), 3.75 (s, 2H), 3.59 (d, J = 4.4 Hz, |
| | | | | 6H), 3.48 (s, 2H), 3.02 (s, 4H), 2.73- |
| | | | | 2.64 (m, 3H), 2.46 (s, 3H), 2.41-2.33 |
| | | | | (m, 8H), 2.26-2.10 (m, 11H), 2.05-1.98 |
| | | | | (m, 4H), 1.87-1.78 (m, 2H), 1.75-1.64 |
| | | | | (m, 6H), 1.54-1.42 (m, 6H), 1.38 (d, J = |
| | | | | 7.2 Hz, 3H), 1.28-1.21 (m, 2H), 0.94 (s, |
| | | | | 9H), 0.89 (s, 3H) |
| I- | A | RM | 1483.6 | 8.98 (s, 1H), 8.35 (d, J = 7.6 Hz, 1H), |
| 172 | | | | 8.08 (d, J = 0.8 Hz, 1H), 7.98-7.94 (m, |
| | | | | 1H), 7.75 (d, J = 9.2 Hz, 1H), 7.67 (d, |
| | | | | J = 8.8 Hz, 2H), 7.58 (d, J = 7.6 Hz, 1H), |
| | | | | 7.48 (d, J = 8.4 Hz, 2H), 7.44-7.41 (m, |
| | | | | 3H), 7.39 (s, 2H), 7.37-7.32 (m, 6H), |
| | | | | 7.30 (d, J = 7.2 Hz, 2H), 7.27 (s, 1H), |
| | | | | 7.21-7.18 (m, 1H), 7.13 (d, J = 6.8 Hz, |
| | | | | 1H), 6.91-6.86 (m, 1H), 6.72 (d, J = 8.4 |
| | | | | Hz, 2H), 5.20 (d, J = 4.4 Hz, 1H), 5.09 |
| | | | | (d, J = 3.2 Hz, 1H), 4.91 (t, J = 7.2 Hz, |
| | | | | 1H), 4.51 (d, J = 9.2 Hz, 1H), 4.41 (t, J = |
| | | | | 7.6 Hz, 1H), 4.30 (d, J = 5.2 Hz, 2H), |
| | | | | 4.05-3.97 (m, 1H), 3.75-3.69 (m, 1H), |
| | | | | 3.60 (s, 2H), 2.45 (s, 3H), 2.27-2.20 (m, |
| | | | | 2H), 2.10 (d, J = 8.0 Hz, 1H), 2.04-1.98 |
| | | | | (m, 2H), 1.93-1.85 (m, 2H), 1.81 (dd, |
| | | | | J = 5.2, 8.0 Hz, 1H), 1.65-1.55 (m, 2H), |
| | | | | 1.52-1.43 (m, 4H), 1.37 (d, J = 7.2 Hz, |
| | | | | 3H), 1.23 (s, 12H), 1.13 (d, J = 4.4 Hz, |
| | | | | 2H), 1.01 (d, J = 12.4 Hz, 2H), 0.93 (s, |
| | | | | 12H), 0.88-0.81 (m, 2H) |
| I- | A | RN | 1469.5 | 12.08-11.94 (m, 1H), 10.10-9.72 (m, |
| 173 | | | | 1H), 9.03-8.96 (m, 1H), 8.37 (d, J = 8.0 |
| | | | | Hz, 1H), 8.18 (s, 1H), 8.02-7.97 (m, 1H), |
| | | | | 7.77 (dd, J = 2.0, 9.2 Hz, 1H), 7.68 |
| | | | | (d, J = 8.8 Hz, 2H), 7.58 (d, J = 7.6 Hz, |
| | | | | 1H), 7.48 (d, J = 8.4 Hz, 2H), 7.45-7.43 |
| | | | | (m, 2H), 7.42-7.39 (m, 2H), 7.38-7.35 |
| | | | | (m, 2H), 7.34 (s, 1H), 7.31 (d, J = 1.6 |
| | | | | Hz, 1H), 7.29 (s, 2H), 7.26-7.22 (m, 2H), |
| | | | | 7.20-7.14 (m, 3H), 6.98 (dd, J = 2.8, 8.8 |
| | | | | Hz, 1H), 6.84 (d, J = 9.2 Hz, 2H), 4.92 (t, |
| | | | | J = 7.2 Hz, 1H), 4.52 (d, J = 9.6 Hz, 1H), |
| | | | | 4.42 (t, J = 8.0 Hz, 1H), 4.30 (d, J = 7.6 |
| | | | | Hz, 2H), 4.17 (dd, J = 1.6, 6.8 Hz, 1H), |
| | | | | 3.90-3.85 (m, 1H), 3.71 (d, J = 12.0 Hz, |
| | | | | 2H), 3.60 (d, J = 4.4 Hz, 2H), 3.22-3.17 |
| | | | | (m, 1H), 3.10-3.05 (m, 1H), 3.03-2.92 |
| | | | | (m, 3H), 2.70-2.67 (m, 3H), 2.59 (s, |
| | | | | 1H), 2.46 (s, 3H), 2.28-2.23 (m, 1H), |
| | | | | 2.14-2.10 (m, 2H), 2.05-2.00 (m, 1H), |
| | | | | 1.89 (d, J = 12.4 Hz, 1H), 1.80 (td, J = |
| | | | | 4.0, 8.4 Hz, 1H), 1.70-1.65 (m, 1H), |
| | | | | 1.57 (d, J = 6.4 Hz, 2H), 1.50-1.44 (m, |
| | | | | 2H), 1.38 (d, J = 7.2 Hz, 3H), 1.25 (s, |
| | | | | 8H), 1.12-1.07 (m, 1H), 0.93 (s, 11H), |
| | | | | 0.85-0.78 (m, 1H) |
| I- | A | RO | 722.6 | 8.98 (s, 1H), 8.37 (s, 1H), 8.07 (s, 1H), |
| 174^{b} | | | | 7.95 (s, 1H), 7.78 (s, 1H), 7.67 (d, J = 8.0 |
| | | | | Hz, 2H), 7.58 (d, J = 5.6 Hz, 1H), 7.48 |
| | | | | (d, J = 7.6 Hz, 2H), 7.43 (d, J = 6.4 Hz, |
| | | | | 3H), 7.39-7.30 (m, 10H), 7.23-7.11 (m, |
| | | | | 3H), 6.92-6.84 (m, 1H), 6.73 (s, 2H), |
| | | | | 5.21 (s, 1H), 5.10 (s, 1H), 4.91 (s, 1H), |
| | | | | 4.51 (d, J = 8.8 Hz, 1H), 4.41 (s, 1H), |
| | | | | 4.29 (s, 2H), 4.03-3.96 (m, 1H), 3.72 |
| | | | | (dd, J = 2.8, 9.2 Hz, 1H), 3.59 (d, J = 1.6 |
| | | | | Hz, 4H), 3.39 (s, 2H), 3.23 (d, J = 3.2 Hz, |
| | | | | 3H), 2.98 (s, 3H), 2.66 (s, 2H), 2.45 (s, |
| | | | | 3H), 2.25 (s, 2H), 2.13 (s, 2H), 2.01 (s, |
| | | | | 2H), 1.91 (s, 1H), 1.79 (s, 1H), 1.47 (s, |
| | | | | 4H), 1.37 (d, J = 5.6 Hz, 3H), 1.23 (s, |
| | | | | 4H), 1.13-1.08 (m, 1H), 0.93 (s, 9H) |
| I- | RP | RQ | 1425.5 | 11.73 (d, J = 13.8 Hz, 2H), 10.24-9.95 |
| 176^{c} | | | | (m, 2H), 8.37 (d, J = 8.0 Hz, 1H), 8.22- |
| | | | | 8.16 (m, 1H), 8.05 (d, J = 2.4 Hz, 1H), |
| | | | | 7.97 (d, J = 9.6 Hz, 1H), 7.84-7.77 (m, |
| | | | | 1H), 7.59 (d, J = 2.4 Hz, 1H), 7.54 (d, |
| | | | | J = 2.4 Hz, 1H), 7.50 (d, J = 8.8 Hz, 1H), |
| | | | | 7.46 (s, 1H), 7.40 (t, J = 7.6 Hz, 4H), |
| | | | | 7.30 (d, J = 8.4 Hz, 2H), 7.16-7.07 (m, |
| | | | | 3H), 6.75-6.68 (m, 1H), 6.42 (d, J = 2.0 |
| | | | | Hz, 1H), 6.24 (s, 1H), 4.84-4.79 (m, |
| | | | | 1H), 4.53-4.49 (m, 1H), 4.44 (t, J = 8.4 |
| | | | | Hz, 1H), 4.27 (s, 1H), 4.13 (s, 1H), 3.60 |
| | | | | (s, 4H), 3.55 (d, J = 6.0 Hz, 5H), 3.05- |
| | | | | 2.94 (m, 4H), 2.75-2.66 (m, 6H), 2.33- |
| | | | | 2.21 (m, 4H), 2.12 (td, J = 7.0, 14.2 Hz, |
| | | | | 2H), 2.01 (s, 3H), 1.96-1.90 (m, 2H), |
| | | | | 1.81-1.74 (m, 1H), 1.60 (d, J = 7.2 Hz, |
| | | | | 2H), 1.55-1.40 (m, 5H), 1.26 (s, 5H), |
| | | | | 0.93 (d, J = 5.2 Hz, 15H) |
| I- | LB | RT | 1495.5 | 11.71-11.64 (m, 1H), 8.48-8.34 (m, 1H), |
| 178 | | | | 8.19-8.11 (m, 1H), 8.03 (d, J = 2.4 Hz, |
| | | | | 1H), 7.91 (d, J = 8.8 Hz, 1H), 7.83- |
| | | | | 7.76 (m, 3H), 7.56-7.41 (m, 6H), 7.41- |
| | | | | 7.33 (m, 3H), 7.06-7.00 (m, 3H), 6.67 (d, |
| | | | | J = 8.4 Hz, 1H), 6.40 (s, 1H), 6.19 (s, |
| | | | | 1H), 5.12 (d, J = 3.6 Hz, 1H), 4.92-4.83 |
| | | | | (m, 2H), 4.52-4.42 (m, 2H), 4.30-4.22 |
| | | | | (m, 1H), 3.65-3.53 (m, 5H), 3.45 (s, 4H), |
| | | | | 3.06 (s, 4H), 2.75 (s, 2H), 2.42 (s, |
| | | | | 6H), 2.29-2.24 (m, 3H), 2.20 (s, 4H), |
| | | | | 2.15-2.09 (m, 3H), 2.03 (d, J = 10.4 Hz, |
| | | | | 1H), 1.96 (s, 2H), 1.84-1.64 (m, 4H), |
| | | | | 1.51-1.43 (m, 4H), 1.39 (t, J = 6.4 Hz, |
| | | | | 2H), 1.28-1.22 (m, 2H), 0.93 (s, 15H) |
| I- | LB | RV | 1504.6 | 11.71-11.63 (m, 1H), 8.36-8.29 (m, |
| 179 | | | | 1H), 8.16-8.09 (m, 1H), 8.06-8.00 (m, |
| | | | | 1H), 7.94-7.86 (m, 1H), 7.83-7.76 (m, |
| | | | | 1H), 7.52-7.45 (m, 3H), 7.38-7.33 (m, |
| | | | | 4H), 7.33-7.30 (m, 3H), 7.07-6.98 (m, |
| | | | | 3H), 6.70-6.63 (m, 1H), 6.41-6.36 (m, |
| | | | | 1H), 6.19 (s, 1H), 5.10 (d, J = 3.6 Hz, |
| | | | | 1H), 4.85-4.78 (m, 1H), 4.76-4.69 (m, |
| | | | | 1H), 4.53-4.48 (m, 1H), 4.47-4.40 (m, |
| | | | | 1H), 4.31-4.23 (m, 1H), 3.60 (s, 2H), |
| | | | | 3.58-3.50 (m, 5H), 3.05 (s, 4H), 2.73 (d, |
| | | | | J = 1.2 Hz, 2H), 2.39 (d, J = 1.6 Hz, 4H), |
| | | | | 2.25 (t, J = 7.2 Hz, 4H), 2.20-2.07 (m, |
| | | | | 8H), 2.03-1.92 (m, 4H), 1.82-1.66 (m, |
| | | | | 4H), 1.54-1.43 (m, 5H), 1.38 (t, J = 6.4 |
| | | | | Hz, 2H), 1.24 (q, J = 7.2 Hz, 3H), 0.92 |
| | | | | (s, 15H) |
| I- | RP | SC | 1437.1 | 11.57 (s, 1H), 8.33 (d, J = 8.0 Hz, 1H), |
| 180^{c} | | | | 8.06 (s, 1H), 7.96 (s, 1H), 7.89-7.83 (m, |
| | | | | 1H), 7.80 (d, J = 9.6 Hz, 1H), 7.53 (d, J = |
| | | | | 8.4 Hz, 1H), 7.46 (d, J = 2.8 Hz, 1H), |
| | | | | 7.41 (d, J = 8.0 Hz, 2H), 7.39-7.26 (m, |
| | | | | 6H), 7.05 (d, J = 8.4 Hz, 2H), 6.98-6.88 |
| | | | | (m, 1H), 6.65-6.61 (m, 1H), 6.60-6.45 |
| | | | | (m, 1H), 6.34 (s, 1H), 6.22 (d, J = 1.2 Hz, |
| | | | | 1H), 5.10 (d, J = 3.6 Hz, 1H), 4.85-4.80 |
| | | | | (m, 1H), 4.74 (t, J = 5.6 Hz, 1H), 4.52 (d, |
| | | | | J = 9.2 Hz, 1H), 4.44 (t, J = 8.0 Hz, 1H), |
| | | | | 4.27 (s, 1H), 4.13 (s, 1H), 3.76-3.66 (m, |
| | | | | 1H), 3.61 (s, 2H), 3.59-3.53 (m, 2H), |
| | | | | 3.02 (s, 5H), 2.73 (s, 3H), 2.25 (dd, J = |
| | | | | 7.2, 14.8 Hz, 2H), 2.19 (d, J = 0.8 Hz, |
| | | | | 4H), 2.15 (d, J = 5.6 Hz, 4H), 2.03-1.98 |
| | | | | (m, 2H), 1.96 (s, 2H), 1.78 (td, J = 4.0, |
| | | | | 8.4 Hz, 1H), 1.60-1.47 (m, 6H), 1.39 (t, |
| | | | | J = 6.4 Hz, 3H), 1.34-1.18 (m, 6H), 0.93 |
| | | | | (d, J = 1.6 Hz, 15H) |
| I- | SG | PY | 1506.6 | 11.74-11.60 (m, 1H), 8.39-8.29 (m, |
| 181^{c} | | | | 1H), 8.18 (s, 1H), 8.06-7.97 (m, 1H), |
| | | | | 7.97-7.85 (m, 1H), 7.83-7.77 (m, 1H), |
| | | | | 7.54-7.44 (m, 3H), 7.43-7.39 (m, 2H), |
| | | | | 7.40-7.23 (m, 5H), 7.04 (d, J = 8.4 Hz, |
| | | | | 2H), 6.98-6.89 (m, 1H), 6.89-6.69 (m, |
| | | | | 1H), 6.68-6.63 (m, 1H), 6.42-6.31 (m, |
| | | | | 1H), 6.23 (s, 1H), 5.10 (d, J = 3.6 Hz, |
| | | | | 1H), 4.87-4.79 (m, 1H), 4.76-4.70 (m, |
| | | | | 1H), 4.55-4.48 (m, 1H), 4.47-4.40 (m, |
| | | | | 1H), 4.33-4.23 (m, 1H), 4.12 (s, 1H), |
| | | | | 3.87-3.67 (m, 1H), 3.61 (br s, 2H), 3.58- |
| | | | | 3.44 (m, 6H), 3.10-3.02 (m, 4H), 2.79- |
| | | | | 2.72 (m, 2H), 2.26 (br d, J = 5.6 Hz, |
| | | | | 4H), 2.24-2.09 (m, 10H), 2.04-1.98 (m, |
| | | | | 2H), 1.96-1.92 (m, 2H), 1.82-1.74 (m, |
| | | | | 2H), 1.47 (td, J = 7.2, 14.3 Hz, 5H), |
| | | | | 1.38 (br t, J = 6.4 Hz, 2H), 1.30-1.20 |
| | | | | (m, 4H), 0.92 (s, 15H) |
| I- | RP | SH | 1480.6 | 11.58 (s, 1H), 8.33 (d, J = 8.0 Hz, 1H), |
| 182^{c} | | | | 8.14 (s, 1H), 8.03 (s, 1H), 7.96 (d, J = |
| | | | | 2.4 Hz, 1H), 7.85-7.76 (m, 2H), 7.54 (d, |
| | | | | J = 8.8 Hz, 1H), 7.45 (t, J = 2.8 Hz, 1H), |
| | | | | 7.41 (d, J = 8.0 Hz, 2H), 7.37 (d, J = |
| | | | | 1.6 Hz, 1H), 7.34 (d, J = 8.4 Hz, 2H), |
| | | | | 7.30 (d, J = 8.0 Hz, 2H), 7.11-7.01 (m, 3H), |
| | | | | 6.84 (d, J = 9.2 Hz, 1H), 6.66-6.59 (m, 1H), |
| | | | | 6.34 (dd, J = 2.0, 3.2 Hz, 1H), 6.22 (d, |
| | | | | J = 1.6 Hz, 1H), 5.20-4.98 (m, 1H), 4.85- |
| | | | | 4.80 (m, 1H), 4.77-4.67 (m, 1H), 4.52 |
| | | | | (d, J = 9.2 Hz, 1H), 4.44 (t, J = 8.0 Hz, |
| | | | | 1H), 4.27 (s, 1H), 4.12 (s, 1H), 3.65- |
| | | | | 3.52 (m, 5H), 3.32 (d, J = 5.4 Hz, 5H), |
| | | | | 3.02 (s, 5H), 2.84-2.77 (m, 2H), 2.73 (s, |
| | | | | 2H), 2.68-2.58 (m, 4H), 2.44 (s, 1H), |
| | | | | 2.26 (d, J = 6.8 Hz, 1H), 2.19 (s, 4H), |
| | | | | 2.14 (d, J = 7.6 Hz, 2H), 2.12-2.06 (m, |
| | | | | 1H), 2.03-1.98 (m, 1H), 1.95 (s, 2H), |
| | | | | 1.79 (J = 4.4, 8.4 Hz, 1H), 1.74-1.67 (m, |
| | | | | 2H), 1.48 (s, 4H), 1.38 (t, J = 6.4 Hz, |
| | | | | 2H), 1.25 (s, 4H), 0.92 (s, 15H) |
| I- | RP | SK | 1423.5 | 11.79-11.69 (m, 2H), 10.62-10.17 (m, |
| 183^{c} | | | | 2H), 8.46-8.32 (m, 1H), 8.19 (s, 1H), |
| | | | | 8.05 (s, 1H), 7.96 (d, J = 10.4 Hz, 1H), |
| | | | | 7.81 (d, J = 9.2 Hz, 1H), 7.59 (d, J = |
| | | | | 1.8 Hz, 1H), 7.55-7.48 (m, 3H), 7.42-7.37 |
| | | | | (m, 4H), 7.30 (d, J = 8.4 Hz, 2H), 7.17- |
| | | | | 7.04 (m, 3H), 6.71 (d, J = 8.8 Hz, 1H), |
| | | | | 6.43 (s, 1H), 6.24 (s, 1H), 4.84-4.78 (m, |
| | | | | 1H), 4.51 (d, J = 8.8 Hz, 1H), 4.44 (t, J = |
| | | | | 8.0 Hz, 1H), 4.26 (s, 1H), 4.13 (s, 1H), |
| | | | | 4.09 (d, J = 5.6 Hz, 1H), 3.95 (d, J = 5.2 |
| | | | | Hz, 2H), 3.83-3.76 (m, 2H), 3.67-3.59 |
| | | | | (m, 6H), 3.57-3.53 (m, 5H), 3.10-2.97 |
| | | | | (m, 4H), 2.72-2.66 (m, 2H), 2.34-2.20 |
| | | | | (m, 4H), 2.16-2.10 (m, 1H), 2.07-1.85 |
| | | | | (m, 4H), 1.81-1.73 (m, 1H), 1.48-1.39 |
| | | | | (m, 6H), 1.31-1.21 (m, 5H), 0.93 (d, |
| | | | | J = 4.4 Hz, 15H) |
| I- | SM | RW | 1520.6 | 11.67 (br s, 1H), 8.34 (d, J = 8.0 Hz, 1H), |
| 184 | | | | 8.12 (s, 1H), 8.02 (d, J = 2.4 Hz, 1H), |
| | | | | 7.90 (br d, J = 8.4 Hz, 1H), 7.81 (br d, |
| | | | | J = 9.2 Hz, 1H), 7.57-7.45 (m, 3H), 7.42 |
| | | | | (d, J = 8.4 Hz, 2H), 7.33 (dd, J = 8.4, 14.4 |
| | | | | Hz, 4H), 7.05 (d, J = 8.4 Hz, 2H), 7.10- |
| | | | | 6.90 (m, 1H), 6.67 (dd, J = 2.0, 9.0 Hz, |
| | | | | 1H), 6.39 (dd, J = 1.6, 3.2 Hz, 1H), 6.21 |
| | | | | (d, J = 1.6 Hz, 1H), 5.10 (d, J = 3.6 Hz, |
| | | | | 1H), 4.87-4.80 (m, 1H), 4.74 (t, J = 6.0 |
| | | | | Hz, 1H), 4.52 (d, J = 9.2 Hz, 1H), 4.45 (t, |
| | | | | J = 8.0 Hz, 1H), 4.28 (br d, J = 2.8 Hz, |
| | | | | 1H), 4.13 (s, 1H), 4.03 (br d, J = 6.0 Hz, |
| | | | | 1H), 3.65-3.53 (m, 4H), 3.50-3.37 (m, 6H), |
| | | | | 3.06 (br s, 4H), 2.75 (br s, 2H), 2.48- |
| | | | | 2.37 (m, 4H), 2.30-2.10 (m, 12H), 2.06- |
| | | | | 1.95 (m, 4H), 1.87-1.74 (m, 2H), 1.72- |
| | | | | 1.62 (m, 1H), 1.57-1.45 (m, 6H), 1.39 |
| | | | | (br t, J = 6.4 Hz, 2H), 1.30-1.21 (m, |
| | | | | 2H), 0.93 (br d, J = 1.6 Hz, 15H) |
| I- | SO | KT | 1563.6 | 11.68-11.61 (m, 1H), 9.02-8.88 (m, |
| 186 | | | | 1H), 8.37 (d, J = 8.0 Hz, 1H), 8.10 (s, |
| | | | | 1H), 8.00 (s, 1H), 7.91-7.85 (m, 1H), |
| | | | | 7.79 (d, J = 9.2 Hz, 1H), 7.51-7.41 (m, |
| | | | | 6H), 7.39-7.32 (m, 5H), 7.04 (d, J = 8.4 |
| | | | | Hz, 2H), 6.98-6.91 (m, 1H), 6.65 (d, J = |
| | | | | 8.8 Hz, 1H), 6.37 (s, 1H), 6.20 (s, 1H), |
| | | | | 5.10 (d, J = 3.2 Hz, 1H), 4.93-4.87 (m, |
| | | | | 1H), 4.58-4.48 (m, 2H), 4.41 (t, J = 8.0 |
| | | | | Hz, 1H), 4.28 (d, J = 1.6 Hz, 1H), 3.59 |
| | | | | (s, 2H), 3.57-3.51 (m, 1H), 3.45 (d, J = |
| | | | | 10.8 Hz, 1H), 3.04 (s, 5H), 2.73 (s, 2H), |
| | | | | 2.45 (s, 3H), 2.21-2.10 (m, 10H), 1.99 |
| | | | | (d, J = 9.2 Hz, 2H), 1.95 (s, 2H), 1.79 |
| | | | | (ddd, J = 4.4, 8.8, 12.8 Hz, 2H), 1.74- |
| | | | | 1.67 (m, 2H), 1.48 (d, J = 6.8 Hz, 5H), |
| | | | | 1.40-1.35 (m, 6H), 1.28-1.21 (m, 3H), |
| | | | | 0.92 (d, J = 2.8 Hz, 18H) |
| I- | SN | KT | 1587.1 | 11.69-11.53 (m, 1H), 8.98 (s, 1H), 8.36 |
| 187^{d} | | | | (d, J = 8.0 Hz, 1H), 8.09 (s, 1H), 7.99 (s, |
| | | | | 1H), 7.91-7.82 (m, 1H), 7.79 (d, J = 9.6 |
| | | | | Hz, 1H), 7.55-7.25 (m, 11H), 7.04 (d, J = |
| | | | | 8.4 Hz, 2H), 6.97-6.88 (m, 1H), 6.65 |
| | | | | (d, J = 8.4 Hz, 1H), 6.37 (s, 1H), 6.20 |
| | | | | (s, 1H), 5.10 (d, J = 3.2 Hz, 1H), 4.91 (t, |
| | | | | J = 6.8 Hz, 1H), 4.58-4.37 (m, 3H), 4.31- |
| | | | | 4.24 (m, 1H), 3.66-3.50 (m, 3H), 3.48- |
| | | | | 3.41 (m, 1H), 3.03 (s, 5H), 2.73 (s, 2H), |
| | | | | 2.45 (s, 3H), 2.27-2.05 (m, 12H), 2.05- |
| | | | | 1.86 (m, 5H), 1.83-1.75 (m, 2H), 1.72- |
| | | | | 1.64 (m, 2H), 1.48 (dd, J = 4.0, 4.8 Hz, |
| | | | | 5H), 1.37 (d, J = 6.8 Hz, 5H), 1.29-1.18 |
| | | | | (m, 3H), 0.92 (d, J = 2.0 Hz, 16H) |
| I- | SQ | SR | 1563.2 | 11.56 (s, 1H), 8.33 (d, J = 8.0 Hz, 1H), |
| 188 | | | | 8.06 (d, J = 1.2 Hz, 1H), 7.96 (d, J = 2.4 |
| | | | | Hz, 1H), 7.86 (d, J = 9.2 Hz, 1H), 7.79 |
| | | | | (d, J = 9.6 Hz, 1H), 7.53 (d, J = 8.8 Hz, |
| | | | | 1H), 7.45 (t, J = 2.8 Hz, 1H), 7.41 (d, J = |
| | | | | 8.4 Hz, 2H), 7.38-7.24 (m, 6H), 7.08 (d, |
| | | | | J = 8.4 Hz, 2H), 6.94 (d, J = 9.2 Hz, 1H), |
| | | | | 6.63 (d, J = 7.2 Hz, 1H), 6.57-6.50 (m, |
| | | | | 1H), 6.34 (dd, J = 2.0, 3.2 Hz, 1H), 6.22 |
| | | | | (s, 1H), 5.09 (d, J = 3.6 Hz, 1H), 4.86- |
| | | | | 4.80 (m, 1H), 4.73 (t, J = 5.6 Hz, 1H), |
| | | | | 4.51 (d, J = 9.2 Hz, 1H), 4.44 (t, J = 8.0 |
| | | | | Hz, 1H), 4.29-4.25 (m, 1H), 4.12 (s, |
| | | | | 1H), 3.70 (d, J = 5.2 Hz, 1H), 3.61 (s, |
| | | | | 2H), 3.58-3.53 (m, 2H), 3.39 (d, J = 0.8 |
| | | | | Hz, 4H), 3.16-3.07 (m, 2H), 3.01 (s, |
| | | | | 4H), 2.79-2.74 (m, 1H), 2.71-2.67 (m, |
| | | | | 2H), 2.58 (s, 4H), 2.46 (s, 2H), 2.40 (s, |
| | | | | 2H), 2.26-2.22 (m, 4H), 2.18 (s, 4H), |
| | | | | 2.13-2.08 (m, 2H), 2.06-1.97 (m, 4H), |
| | | | | 1.86 (d, J = 16.8 Hz, 1H), 1.81-1.75 (m, |
| | | | | 1H), 1.67-1.58 (m, 2H), 1.58-1.50 (m, |
| | | | | 2H), 1.49-1.40 (m, 5H), 1.24 (d, J = 3.2 |
| | | | | Hz, 5H), 0.93 (s, 9H), 0.90 (s, 3H) |
| I- | C | OF | 1514.2 | 8.98 (s, 1H), 8.38-8.33 (m, 1H), 8.13 (s, |
| 192 | | | | 1H), 7.92 (s, 1H), 7.83-7.78 (m, 1H), |
| | | | | 7.72 (br d, J = 8.4 Hz, 2H), 7.44-7.41 |
| | | | | (m, 2H), 7.37 (d, J = 8.4 Hz, 4H), 7.34- |
| | | | | 7.31 (m, 2H), 7.26 (t, J = 7.6 Hz, 2H), |
| | | | | 7.18 (br d, J = 7.6 Hz, 1H), 7.12 (br d, |
| | | | | J = 8.4 Hz, 2H), 6.89-6.85 (m, 2H), 5.11- |
| | | | | 5.06 (m, 1H), 4.93-4.88 (m, 1H), 4.51 |
| | | | | (d, J = 9.2 Hz, 1H), 4.46-4.38 (m, 1H), |
| | | | | 4.25 (s, 1H), 4.12-4.04 (m, 1H), 3.62- |
| | | | | 3.57 (m, 2H), 3.36 (br s, 2H), 3.24-3.17 |
| | | | | (m, 4H), 2.68-2.66 (m, 2H), 2.45 (s, |
| | | | | 3H), 2.34-2.32 (m, 3H), 2.23 (br d, J = |
| | | | | 7.6 Hz, 10H), 2.01-1.96 (m, 6H), 1.83- |
| | | | | 1.76 (m, 2H), 1.70-1.66 (m, 2H), 1.46- |
| | | | | 1.42 (m, 3H), 1.37 (d, J = 7.2 Hz, 3H), |
| | | | | 1.26-1.20 (m, 7H), 0.97 (s, 6H), 0.93 |
| | | | | (s, 9H) |
| I- | C | MZ | 793.0 | 8.99 (s, 1H), 8.37 (d, J = 7.6 Hz, 1H), |
| 193 | | | (M/2 + H) | 8.13 (s, 1H), 7.97-7.93 (m, 1H), 7.78 (d, |
| | | | | J = 9.2 Hz, 1H), 7.72 (br d, J = 9.2 Hz, |
| | | | | 2H), 7.45-7.43 (m, 2H), 7.38 (d, J = 8.4 |
| | | | | Hz, 4H), 7.35-7.32 (m, 2H), 7.27 (t, J = |
| | | | | 7.6 Hz, 2H), 7.19 (d, J = 7.6 Hz, 1H), |
| | | | | 7.13 (br d, J = 8.4 Hz, 2H), 7.03-6.96 |
| | | | | (m, 1H), 6.88-6.84 (m, 2H), 5.09 (d, J = |
| | | | | 3.6 Hz, 1H), 4.92 (t, J = 7.4 Hz, 1H), 4.52 |
| | | | | (d, J = 9.2 Hz, 1H), 4.42 (t, J = 8.2 Hz, |
| | | | | 1H), 4.30-4.27 (m, 1H), 4.12-4.05 (m, |
| | | | | 1H), 3.61 (br d, J = 4.0 Hz, 2H), 3.39- |
| | | | | 3.35 (m, 4H), 3.25-3.17 (m, 4H), 2.53 |
| | | | | (d, J = 2.0 Hz, 12H), 2.46 (s, 3H), 2.27- |
| | | | | 2.20 (m, 8H), 2.14-2.08 (m, 2H), 2.00 |
| | | | | (br s, 4H), 1.80 (ddd, J = 4.8, 8.0, 12.8 |
| | | | | Hz, 2H), 1.47-1.42 (m, 6H), 1.38 (d, J = |
| | | | | 6.8 Hz, 3H), 1.23 (br s, 8H), 0.98 (s, 6H), |
| | | | | 0.94 (s, 9H) |
| I- | C | 0 | 1526.3 | 8.99-8.94 (m, 1H), 8.54-8.42 (m, 1H), |
| 194 | | | | 8.12 (d, J = 1.6 Hz, 1H), 7.95 (dd, J = 2.0, |
| | | | | 9.2 Hz, 1H), 7.71 (d, J = 8.8 Hz, 2H), |
| | | | | 7.44 (d, J = 2.8 Hz, 1H), 7.42-7.41 (m, |
| | | | | 1H), 7.47-7.30 (m, 1H), 7.29-7.23 (m, |
| | | | | 2H), 7.21-7.08 (m, 3H), 7.00 (br d, J = |
| | | | | 9.2 Hz, 1H), 6.86 (br d, J = 8.4 Hz, 3H), |
| | | | | 6.05 (d, J = 10.0 Hz, 1H), 5.11 (dd, J = |
| | | | | 3.6, 6.8 Hz, 1H), 4.47-4.22 (m, 4H), |
| | | | | 4.19-3.99 (m, 3H), 3.81-3.70 (m, 1H), |
| | | | | 3.66 (d, J = 9.6 Hz, 1H), 3.60-3.36 (m, |
| | | | | 5H), 3.28-3.12 (m, 6H), 3.10-2.71 (m, |
| | | | | 3H), 2.67 (td, J = 2.0, 3.6 Hz, 1H), 2.48- |
| | | | | 2.42 (m, 5H), 2.42-2.36 (m, 4H), 2.35- |
| | | | | 2.31 (m, 3H), 2.29-2.15 (m, 6H), 2.14- |
| | | | | 1.96 (m, 5H), 1.95-1.68 (m, 5H), 1.43 |
| | | | | (br t, J = 6.0 Hz, 2H), 0.93 (s, 9H), 0.80 |
| | | | | (dd, J = 6.8, 13.6 Hz, 3H), 0.67 (d, J = |
| | | | | 6.8 Hz, 1H), 0.57 (d, J = 6.8 Hz, 1H) |
| I- | C | P | 1578.5 | 9.00-8.95 (m, 1H), 8.51 (t, J = 6.0 Hz, |
| 195^{d} | | | | 1H), 8.11 (s, 1H), 7.96-7.92 (m, 1H), |
| | | | | 7.71 (d, J = 8.8 Hz, 2H), 7.45-7.30 (m, |
| | | | | 10H), 7.26 (t, J = 7.6 Hz, 2H), 7.19-7.15 |
| | | | | (m, 1H), 7.12 (d, J = 8.4 Hz, 2H), 7.02- |
| | | | | 6.95 (m, 1H), 6.85 (d, J = 8.4 Hz, 3H), |
| | | | | 6.06 (s, 1H), 5.14-5.08 (m, 1H), 4.39- |
| | | | | 4.29 (m, 4H), 4.17-4.01 (m, 4H), 3.79- |
| | | | | 3.73 (m, 1H), 3.66 (d, J = 9.6 Hz, 1H), |
| | | | | 3.59-3.53 (m, 1H), 3.44 (dd, J = 5.2, |
| | | | | 10.3 Hz, 2H), 3.24-3.10 (m, 5H), 2.46- |
| | | | | 2.43 (m, 4H), 2.23 (dd, J = 2.0, 4.4 Hz, |
| | | | | 10H), 1.99 (s, 4H), 1.92-1.88 (m, 1H), |
| | | | | 1.72-1.59 (m, 3H), 1.53-1.39 (m, 6H), |
| | | | | 1.37-1.19 (m, 6H), 1.05 (t, J = 7.2 Hz, |
| | | | | 1H), 0.99-0.93 (m, 10H), 0.87-0.76 (m, |
| | | | | 4H) |
| I- | C | E | 1576.0 | 12.32-11.93 (m, 1H), 10.15-9.67 (m, |
| 196^{e} | | | | 1H), 9.60-9.29 (m, 1H), 8.98 (s, 1H), |
| | | | | 8.49 (br t, J = 6.0 Hz, 1H), 8.18 (d, J = |
| | | | | 1.6 Hz, 1H), 8.08-7.92 (m, 1H), 7.77 (br |
| | | | | d, J = 8.4 Hz, 2H), 7.45-7.36 (m, 3H), |
| | | | | 7.30-7.20 (m, 5H), 7.18-7.10 (m, 4H), |
| | | | | 7.03-6.92 (m, 5H), 4.59 (br d, J = 9.2 |
| | | | | Hz, 1H), 4.52 (br t, J = 8.3 Hz, 1H), 4.35 |
| | | | | (br s, 1H), 4.33-4.18 (m, 2H), 4.16- |
| | | | | 3.99 (m, 4H), 3.97-3.84 (m, 2H), 3.68- |
| | | | | 3.60 (m, 4H), 3.39-3.35 (m, 4H), 3.26- |
| | | | | 2.73 (m, 12H), 2.56 (br d, J = 7.2 Hz, |
| | | | | 2H), 2.45 (s, 3H), 2.28 (br s, 2H), 2.18- |
| | | | | 1.86 (m, 9H), 1.48 (br t, J = 5.6 Hz, 2H), |
| | | | | 1.41-1.28 (m, 2H), 1.22 (br d, J = 8.4 |
| | | | | Hz, 2H), 1.03-0.97 (m, 6H), 0.95 (s, 9H) |
| I- | C | F | 1617.5 | 12.29-12.01 (m, 1H), 9.81-9.58 (m, |
| 197 | | | | 1H), 9.45-9.18 (m, 1H), 8.98 (s, 1H), |
| | | | | 8.55-8.42 (m, 1H), 8.18 (d, J = 2.0 Hz, |
| | | | | 1H), 8.00 (dd, J = 1.6, 9.2 Hz, 1H), 7.77 |
| | | | | (br d, J = 8.8 Hz, 2H), 7.45-7.37 (m, |
| | | | | 3H), 7.32-7.20 (m, 5H), 7.18-7.11 (m, |
| | | | | 4H), 7.02-6.91 (m, 5H), 4.60 (br d, J = |
| | | | | 9.2 Hz, 1H), 4.52 (br t, J = 8.4 Hz, 1H), |
| | | | | 4.35 (br s, 1H), 4.31 (br s, 1H), 4.17 (br |
| | | | | d, J = 5.6 Hz, 1H), 4.04 (br t, J = 5.6 Hz, |
| | | | | 4H), 3.92 (br d, J = 5.6 Hz, 1H), 3.65- |
| | | | | 3.59 (m, 4H), 3.46-3.28 (m, 7H), 3.26- |
| | | | | 2.70 (m, 9H), 2.45 (s, 3H), 2.34 (br s, |
| | | | | 2H), 2.28 (br s, 2H), 2.21-2.00 (m, 6H), |
| | | | | 1.92 (dt, J = 4.0, 8.7 Hz, 1H), 1.81-1.72 |
| | | | | (m, 2H), 1.54-1.42 (m, 6H), 1.41-1.32 |
| | | | | (m, 4H), 1.22 (br dd, J = 2.8, 8.3 Hz, 2H), |
| | | | | 1.01 (s, 6H), 0.95 (s, 9H) |
| I- | C | SU | 1546.3 | 8.97 (s, 1H), 8.56 (s, 1H), 8.14 (s, 1H), |
| 198 | | | | 8.07 (d, J = 1.6 Hz, 1H), 7.92 (dd, J = 1.6, |
| | | | | 9.2 Hz, 1H), 7.71 (d, J = 8.8 Hz, 2H), |
| | | | | 7.39-7.34 (m, 5H), 7.28 (br t, J = 7.6 Hz, |
| | | | | 3H), 7.20-7.16 (m, 1H), 7.12 (d, J = 8.4 |
| | | | | Hz, 2H), 6.97-6.93 (m, 2H), 6.87 (br d, |
| | | | | J = 9.6 Hz, 1H), 6.80-6.74 (m, 3H), 5.16 |
| | | | | (br s, 1H), 4.92 (s, 2H), 4.59 (d, J = 9.2 |
| | | | | Hz, 1H), 4.50 (t, J = 8.4 Hz, 1H), 4.35- |
| | | | | 4.29 (m, 2H), 4.28-4.21 (m, 1H), 4.06- |
| | | | | 4.01 (m, 1H), 3.64 (br d, J = 3.6 Hz, 1H), |
| | | | | 3.61-3.57 (m, 1H), 3.48-3.43 (m, 2H), |
| | | | | 3.26 (br s, 6H), 3.13 (br s, 4H), 2.42 (s, |
| | | | | 6H), 2.36-2.31 (m, 4H), 2.22 (br s, 4H), |
| | | | | 2.09-2.04 (m, 1H), 1.98 (br s, 3H), 1.95- |
| | | | | 1.86 (m, 2H), 1.72 (br dd, J = 6.0, 12.8 |
| | | | | Hz, 1H), 1.45-1.41 (m, 2H), 1.37 (br s, |
| | | | | 1H), 1.35-1.31 (m, 1H), 1.21 (br dd, J = |
| | | | | 2.8, 8.4 Hz, 2H), 0.96 (d, J = 3.2 Hz, |
| | | | | 15H) |
| I- | C | AW | 1614.3 | 9.00-8.94 (m, 1H), 8.46 (brt, J = 5.6 Hz, |
| 201 | | | | 1H), 8.08 (s, 1H), 7.93 (br d, J = 8.8 Hz, |
| | | | | 1H), 7.71 (d, J = 8.8 Hz, 2H), 7.41-7.33 |
| | | | | (m, 6H), 7.31-7.27 (m, 3H), 7.22-7.17 |
| | | | | (m, 1H), 7.13-7.09 (m, 3H), 6.93 (br d, |
| | | | | J = 9.2 Hz, 2H), 6.81 (br d, J = 8.8 Hz, |
| | | | | 3H), 5.17 (d, J = 3.6 Hz, 1H), 4.60 (br d, |
| | | | | J = 9.2 Hz, 1H), 4.53-4.47 (m, 1H), 4.42- |
| | | | | 4.34 (m, 2H), 4.25-4.19 (m, 1H), 4.09- |
| | | | | 4.00 (m, 1H), 3.68-3.58 (m, 2H), 3.51- |
| | | | | 3.40 (m, 4H), 3.15 (br s, 4H), 3.10 (br |
| | | | | d, J = 7.2 Hz, 2H), 2.74 (br dd, J = 2.0, |
| | | | | 5.6 Hz, 2H), 2.45 (s, 3H), 2.23 (br d, J = |
| | | | | 6.8 Hz, 4H), 2.14-2.07 (m, 4H), 2.01- |
| | | | | 1.94 (m, 4H), 1.73-1.68 (m, 2H), 1.51 |
| | | | | (br d, J = 12.0 Hz, 4H), 1.46-1.32 (m, |
| | | | | 6H), 1.24-1.21 (m, 2H), 1.18 (t, J = 7.2 |
| | | | | Hz, 5H), 0.96 (s, 18H) |
| I- | C | AX | 1614.3 | 8.97 (s, 1H), 8.55 (br t, J = 6.0 Hz, 1H), |
| 202 | | | | 8.08 (s, 1H), 7.93 (dd, J = 1.2, 9.6 Hz, |
| | | | | 1H), 7.71 (d, J = 8.8 Hz, 2H), 7.43 (d, |
| | | | | J = 8.0 Hz, 1H), 7.38-7.32 (m, 5H), 7.31- |
| | | | | 7.26 (m, 3H), 7.19 (br d, J = 7.2 Hz, |
| | | | | 1H), 7.11 (d, J = 8.4 Hz, 2H), 7.00 (s, |
| | | | | 1H), 6.90 (br d, J = 8.0 Hz, 2H), 6.80 (br |
| | | | | d, J = 8.8 Hz, 3H), 5.18 (d, J = 3.6 Hz, |
| | | | | 1H), 4.77 (br s, 1H), 4.60 (br d, J = 9.2 |
| | | | | Hz, 1H), 4.53 (t, J = 8.0 Hz, 1H), 4.39- |
| | | | | 4.33 (m, 2H), 4.22-4.14 (m, 1H), 4.06- |
| | | | | 3.99 (m, 1H), 3.68-3.59 (m, 2H), 3.44 |
| | | | | (br s, 4H), 3.16 (br s, 4H), 3.12 (br s, 2H), |
| | | | | 2.76 (br s, 2H), 2.44 (s, 3H), 2.28 (br s, |
| | | | | 4H), 2.22 (br s, 4H), 1.98 (br s, 5H), |
| | | | | 1.77-1.62 (m, 6H), 1.48-1.37 (m, 6H), 1.24- |
| | | | | 1.20 (m, 2H), 1.17 (t, J = 7.2 Hz, 3H), |
| | | | | 0.96 (br d, J = 1.6 Hz, 18H) |
| I- | C | AZ | 1587.5 | 8.97 (s, 1H), 8.48 (br t, J = 5.6 Hz, 1H), |
| 203 | | | | 8.09 (s, 1H), 7.93 (br d, J = 9.6 Hz, 1H), |
| | | | | 7.71 (br d, J = 8.8 Hz, 2H), 7.41 (d, J = |
| | | | | 8.0 Hz, 1H), 7.38 (s, 1H), 7.36 (s, 2H), |
| | | | | 7.33 (s, 2H), 7.29-7.25 (m, 3H), 7.18 (br |
| | | | | d, J = 7.2 Hz, 1H), 7.11 (d, J = 8.4 Hz, |
| | | | | 2H), 6.95 (br d, J = 6.8 Hz, 2H), 6.82 (br |
| | | | | dd, J = 2.8, 4.0 Hz, 2H), 6.72 (s, 1H), |
| | | | | 5.16 (d, J = 4.0 Hz, 1H), 4.80-4.75 (m, |
| | | | | 1H), 4.59 (d, J = 8.8 Hz, 1H), 4.52 (t, J = |
| | | | | 8.4 Hz, 1H), 4.34 (br d, J = 5.2 Hz, 1H), |
| | | | | 4.27 (br dd, J = 6.4, 11.6 Hz, 2H), 4.08- |
| | | | | 4.03 (m, 1H), 3.68-3.56 (m, 4H), 3.48- |
| | | | | 3.37 (m, 2H), 3.14 (br dd, J = 4.0, 7.2 Hz, |
| | | | | 4H), 2.77-2.72 (m, 2H), 2.44 (s, 4H), |
| | | | | 2.23 (br d, J = 4.4 Hz, 6H), 2.13-2.04 |
| | | | | (m, 2H), 1.98 (br s, 3H), 1.96-1.89 (m, |
| | | | | 2H), 1.76-1.68 (m, 1H), 1.43 (br t, J = |
| | | | | 5.6 Hz, 2H), 1.40-1.37 (m, 1H), 1.35- |
| | | | | 1.32 (m, 1H), 1.27-1.24 (m, 6H), 1.23 |
| | | | | (br d, J = 2.0 Hz, 2H), 1.21-1.19 (m, |
| | | | | 1H), 1.18-1.14 (m, 1H), 0.96 (s, 15H) |
| I- | C | RB | 1528.3 | 8.98 (s, 1H), 8.56 (t, J = 5.6 Hz, 1H), 8.13 |
| 205 | | | | (s, 1H), 7.94 (d, J = 8.0 Hz, 1H), 7.83 (d, |
| | | | | J = 8.0 Hz, 1H), 7.71 (d, J = 8.8 Hz, 1H), |
| | | | | 7.50-7.39 (m, 5H), 7.33-7.25 (m, 4H), |
| | | | | 7.13-7.16 (m, 3H), 7.10-7.00 (m, 1H), |
| | | | | 6.87-6.85 (m, 3H), 5.11 (d, J = 3.2 Hz, |
| | | | | 1H), 4.53 (d, J = 9.6 Hz, 1H), 4.50-4.44 |
| | | | | (m, 2H), 4.43-4.42 (m, 1H), 4.36-4.33 |
| | | | | (m, 1H), 3.66-3.64 (m, 2H), 3.30-3.10 |
| | | | | (m, 7H), 2.67 (s, 1H), 2.60-2.46 (m, |
| | | | | 11H), 2.43 (s, 2H), 2.40-2.25 (m, 9H), |
| | | | | 2.20-1.90 (m, 6H), 1.55-1.45 (m, 6H), |
| | | | | 1.25-1.23 (m, 2H), 0.97 (s, 6H), 0.93 |
| | | | | (s, 9H) |
| I- | C | SW | 1568.6 | 8.98 (s, 1H), 8.37 (br d, J = 7.6 Hz, 1H), |
| 207 | | | | 8.12 (br s, 1H), 7.95 (br d, J = 9.0 Hz, |
| | | | | 1H), 7.71 (br d, J = 8.4 Hz, 2H), 7.65 (br |
| | | | | d, J = 9.0 Hz, 1H), 7.48-7.30 (m, 9H), 7.26 |
| | | | | (br t, J = 7.4 Hz, 2H), 7.18 (br d, J = |
| | | | | 7.2 Hz, 1H), 7.12 (br d, J = 8.0 Hz, 2H), |
| | | | | 7.04-6.96 (m, 1H), 6.93-6.81 (m, 3H), |
| | | | | 5.10 (br d, J = 2.8 Hz, 1H), 4.96-4.87 |
| | | | | (m, 1H), 4.48 (br d, J = 9.4 Hz, 1H), 4.41 |
| | | | | (br t, J = 7.8 Hz, 1H), 4.27 (br s, 1H), |
| | | | | 4.13-4.05 (m, 1H), 3.61-3.52 (m, 2H), |
| | | | | 3.31-3.15 (m, 11H), 2.67 (br s, 1H), |
| | | | | 2.45 (br s, 3H), 2.34-2.19 (m, 9H), 2.14 |
| | | | | (br d, J = 4.4 Hz, 3H), 2.04-1.92 (m, |
| | | | | 5H), 1.85-1.52 (m, 9H), 1.48-1.22 (m, |
| | | | | 9H), 0.95 (br d, J = 17.6 Hz, 15H) |
| I- | DP | KT | 1337.0 | 0.94 (d, J = 6.25 Hz, 15 H) 1.20-1.27 (m, |
| 212^{f} | | | | 3 H) 1.37 (br d, J = 7.00 Hz, 9 H) 1.41- |
| | | | | 1.48 (m, 4 H) 1.71-1.86 (m, 2 H) 1.95- |
| | | | | 2.03 (m, 2 H) 2.05-2.17 (m, 2 H) 2.22 (br t, |
| | | | | J = 7.38 Hz, 4 H) 2.45 (s, 5 H) 3.22- |
| | | | | 3.31 (m, 9 H) 3.60 (br s, 2 H) 4.08-4.13 |
| | | | | (m, 1 H) 4.27 (br d, J = 1.75 Hz, 1 H) 4.41 |
| | | | | (t, J = 7.88 Hz, 1 H) 4.51 (d, J = 9.26 Hz, 1 |
| | | | | H) 4.89-4.95 (m, 1 H) 5.09 (d, J = 3.25 |
| | | | | Hz, 1 H) 6.90 (br d, J = 8.75 Hz, 2 H) 7.05 |
| | | | | (br dd, J = 10.38, 1.63 Hz, 1 H) 7.14-7.20 |
| | | | | (m, 1 H) 7.24-7.28 (m, 2 H) 7.31-7.33 |
| | | | | (m, 2 H) 7.36-7.39 (m, 2 H) 7.42-7.44 |
| | | | | (m, 2 H) 7.71 (d, J = 9.01 Hz, 2 H) 7.78 |
| | | | | (br d, J = 9.26 Hz, 1 H) 7.95-7.99 (m, 1 |
| | | | | H) 8.15 (d, J = 1.75 Hz, 1 H) 8.36 (d, |
| | | | | J = 7.75 Hz, 1 H) 8.98 (s, 1 H) |
| I- | DP | KT | 1337.0 | 0.93 (d, J = 5.50 Hz, 15 H) 1.22-1.26 (m, |
| 213^{f} | | | | 3 H) 1.35-1.40 (m, 9 H) 1.42-1.47 (m, |
| | | | | 4 H) 1.75-1.83 (m, 2 H) 1.97-2.02 (m, |
| | | | | 2 H) 2.07-2.12 (m, 2 H) 2.19-2.24 (m, |
| | | | | 4 H) 2.45 (s, 5 H) 3.25-3.29 (m, 9 H) |
| | | | | 3.60 (br d, J = 2.75 Hz, 2 H) 4.04-4.12 |
| | | | | (m, 1 H) 4.25-4.30 (m, 1 H) 4.39-4.45 |
| | | | | (m, 1 H) 4.48-4.53 (m, 1 H) 4.87-4.96 |
| | | | | (m, 1 H) 5.08-5.11 (m, 1 H) 6.88 (br d, |
| | | | | J = 8.00 Hz, 3 H) 7.15-7.20 (m, 1 H) 7.27 |
| | | | | (t, J = 7.38 Hz, 2 H) 7.32-7.34 (m, 2 H) |
| | | | | 7.36-7.39 (m, 2 H) 7.42-7.45 (m, 2 H) |
| | | | | 7.71 (d, J = 8.75 Hz, 2 H) 7.79 (d, J = 9.01 |
| | | | | Hz, 1 H) 7.95 (dd, J = 9.38, 1.38 Hz, 1 H) |
| | | | | 8.12 (s, 1 H) 8.35-8.39 (m, 1 H) 8.98 (s, |
| | | | | 1 H) |
| I- | SX | W | 760.6 | 8.96 (s, 1H), 8.49 (br t, J = 6.0 Hz, 1H), |
| 215^{b} | | | | 8.13 (s, 1H), 8.11 (br s, 1H), 7.95-7.91 |
| | | | | (m, 1H), 7.71 (br d, J = 8.4 Hz, 2H), 7.42- |
| | | | | 7.35 (m, 4H), 7.29 (br t, J = 7.2 Hz, 4H), |
| | | | | 7.22 (br d, J = 7.6 Hz, 2H), 7.12 (br d, |
| | | | | J = 8.4 Hz, 3H), 6.98-6.92 (m, 3H), 6.85- |
| | | | | 6.81 (m, 2H), 5.16 (br d, J = 2.8 Hz, |
| | | | | 1H), 4.59 (br d, J = 9.2 Hz, 1H), 4.51 (br |
| | | | | t, J = 8.4 Hz, 1H), 4.34 (br s, 1H), 4.29- |
| | | | | 4.22 (m, 2H), 4.01 (br d, J = 4.4 Hz, 2H), |
| | | | | 3.97-3.87 (m, 2H), 3.66-3.59 (m, 4H), |
| | | | | 3.19 (br d, J = 1.6 Hz, 2H), 2.90 (s, 3H), |
| | | | | 2.84-2.82 (m, 2H), 2.74 (br s, 2H), 2.44 |
| | | | | (br s, 6H), 2.24-2.20 (m, 4H), 2.10- |
| | | | | 2.05 (m, 2H), 1.99 (br s, 3H), 1.93-1.87 |
| | | | | (m, 4H), 1.45-1.42 (m, 2H), 1.38 (br d, |
| | | | | J = 10.0 Hz, 1H), 1.35-1.32 (m, 1H), |
| | | | | 1.24-1.19 (m, 3H), 0.97 (s, 6H), 0.94 (br |
| | | | | s, 9H) |
| I- | C | BO | 1472.6 | 8.53 (t, J = 5.4 Hz, 1H), 8.13 (s, 1H), 7.96 |
| 216 | | | | (dd, J = 1.8, 9.2 Hz, 1H), 7.72 (br d, J = |
| | | | | 8.8 Hz, 2H), 7.37 (d, J = 8.4 Hz, 2H), |
| | | | | 7.35-7.31 (m, 3H), 7.31-7.22 (m, 4H), |
| | | | | 7.20-7.15 (m, 1H), 7.12 (d, J = 8.4 Hz, |
| | | | | 2H), 7.05-7.00 (m, 2H), 6.97 (d, J = 8.0 |
| | | | | Hz, 1H), 6.86 (br d, J = 8.8 Hz, 2H), 5.15 |
| | | | | (d, J = 3.6 Hz, 1H), 4.89 (s, 2H), 4.59 (d, |
| | | | | J = 9.2 Hz, 1H), 4.48 (t, J = 8.4 Hz, 1H), |
| | | | | 4.36-4.31 (m, 1H), 4.30-4.19 (m, 2H), |
| | | | | 4.14-4.12 (m, 1H), 3.69-3.54 (m, 3H), |
| | | | | 3.48-3.33 (m, 9H), 3.25 (br d, J = 19.2 |
| | | | | Hz, 8H), 2.54-2.51 (m, 3H), 2.23 (br s, |
| | | | | 3H), 2.12-2.02 (m, 2H), 1.99 (br s, 3H), |
| | | | | 1.88 (ddd, J = 4.4, 8.8, 12.8 Hz, 1H), 1.84- |
| | | | | 1.74 (m, 1H), 1.44 (br t, J = 6.4 Hz, 2H), |
| | | | | 1.41-1.32 (m, 2H), 1.24-1.19 (m, 2H), |
| | | | | 0.98-0.93 (m, 15H) |
| I- | C | CK | 1448.8 | 0.92-0.99 (m, 15 H), 1.11-1.28 (m, 3 |
| 217 | | | | H), 1.32-1.47 (m, 4 H), 1.76-1.84 (m, |
| | | | | 1 H), 1.89 (ddd, J = 12.8, 8.4, 4.4 Hz, 1 |
| | | | | H), 2.00 (br s, 3 H), 2.04-2.09 (m, 1 H), |
| | | | | 2.14-2.42 (m, 11 H), 2.77-2.97 (m, 2 |
| | | | | H), 3.19-3.27 (m, 5 H), 3.38-3.51 (m, |
| | | | | 5 H), 3.57-3.67 (m, 2 H), 4.11 (br d, J = |
| | | | | 2.0 Hz, 1 H), 4.27 (br t, J = 5.2 Hz, 2 H), |
| | | | | 4.33 (br s, 1 H), 4.49 (t, J = 8.0 Hz, 1 H), |
| | | | | 4.59 (d, J = 9.2 Hz, 1 H), 4.84 (s, 2 H), |
| | | | | 5.15 (d, J = 3.6 Hz, 1 H), 6.83-6.94 (m, |
| | | | | 5 H), 7.02 (br d, J = 8.8 Hz, 1 H), 7.11- |
| | | | | 7.19 (m, 4 H), 7.24-7.35 (m, 6 H), 7.38 |
| | | | | (d, J = 8.4 Hz, 2 H), 7.72 (br d, J = 8.8 Hz, |
| | | | | 2 H), 7.96 (dd, J = 9.2, 2.0 Hz, 1 H), 8.13 |
| | | | | (s, 1 H), 8.49 (br t, J = 5.6 Hz, 1 H) |
| I- | C | DW | 1560.4 | 8.96 (s, 1H), 8.58 (br d, J = 8.0 Hz, 1H), |
| 219 | | | | 8.12 (s, 1H), 7.94 (br d, J = 9.2 Hz, 1H), |
| | | | | 7.71 (br d, J = 8.8 Hz, 2H), 7.52 (d, J = |
| | | | | 8.0 Hz, 1H), 7.37 (br d, J = 8.0 Hz, 2H), |
| | | | | 7.35-7.31 (m, 2H), 7.29-7.23 (m, 3H), |
| | | | | 7.17 (br d, J = 7.6 Hz, 1H), 7.12 (br d, |
| | | | | J = 8.4 Hz, 2H), 7.04-6.98 (m, 1H), 6.96- |
| | | | | 6.91 (m, 2H), 6.85 (br d, J = 9.2 Hz, |
| | | | | 2H), 5.14 (d, J = 3.6 Hz, 1H), 5.05-4.95 |
| | | | | (m, 2H), 4.91-4.83 (m, 1H), 4.58-4.48 |
| | | | | (m, 2H), 4.34 (br d, J = 1.6 Hz, 1H), |
| | | | | 4.14-4.02 (m, 1H), 3.64-3.54 (m, 2H), |
| | | | | 3.49-3.34 (m, 4H), 3.23-3.01 (m, 5H), |
| | | | | 2.81-2.70 (m, 1H), 2.42 (s, 8H), 2.23 |
| | | | | (br s, 6H), 2.10-2.03 (m, 2H), 1.99 (br s, 5H), |
| | | | | 1.80-1.73 (m, 1H), 1.43 (br t, J = 6.4 Hz, |
| | | | | 2H), 1.35 (br d, J = 6.8 Hz, 5H), 1.26- |
| | | | | 1.14 (m, 3H), 0.97 (s, 6H), 0.88 (s, 9H) |
| I- | C | SZ | 1511.2 | 8.97 (s, 1H), 8.34 (t, J = 5.6 Hz, 1H), 8.13 |
| 221^{g} | | | | (s, 1H), 7.97-7.93 (m, 1H), 7.71 (br d, J = |
| | | | | 8.8 Hz, 2H), 7.37 (d, J = 8.4 Hz, 2H), |
| | | | | 7.34-7.31 (m, 2H), 7.29-7.24 (m, 3H), |
| | | | | 7.17 (br d, J = 7.2 Hz, 1H), 7.12 (br d, |
| | | | | J = 8.4 Hz, 2H), 7.01 (br d, J = 8.0 Hz, 1H), |
| | | | | 6.98-6.94 (m, 2H), 6.85 (br d, J = 8.8 |
| | | | | Hz, 2H), 6.21 (s, 1H), 5.11 (d, J = 3.6 Hz, |
| | | | | 1H), 4.99-4.89 (m, 2H), 4.45 (br t, J = |
| | | | | 7.6 Hz, 1H), 4.34-4.24 (m, 3H), 4.13- |
| | | | | 4.06 (m, 1H), 3.84 (d, J = 8.4 Hz, 1H), |
| | | | | 3.55 (br d, J = 5.2 Hz, 2H), 3.46-3.37 |
| | | | | (m, 4H), 3.34 (br d, J = 4.8 Hz, 2H), |
| | | | | 3.29-3.17 (m, 6H), 2.84-2.70 (m, 1H), 2.44- |
| | | | | 2.40 (m, 6H), 2.26-2.19 (m, 6H), 2.17 |
| | | | | (s, 1H), 2.13 (s, 3H), 2.09-2.02 (m, 2H), |
| | | | | 1.99 (br s, 3H), 1.93-1.88 (m, 1H), 1.86- |
| | | | | 1.67 (m, 2H), 1.43 (br t, J = 6.0 Hz, 2H), |
| | | | | 1.23 (s, 1H), 0.98-0.96 (m, 8H), 0.80 (d, |
| | | | | J = 6.8 Hz, 2H), 0.68-0.64 (m, 1H), 0.51 |
| | | | | (d, J = 6.8 Hz, 1H) |
| I- | C | SZ | 1512.2 | 8.98 (s, 1H), 8.42 (br t, J = 5.6 Hz, 1H), |
| 222^{g} | | | | 8.12 (s, 1H), 7.96-7.93 (m, 1H), 7.71 (br |
| | | | | d, J = 8.4 Hz, 2H), 7.37 (br d, J = 8.4 Hz, |
| | | | | 2H), 7.33 (br d, J = 7.2 Hz, 3H), 7.26 (br |
| | | | | t, J = 7.6 Hz, 2H), 7.19-7.15 (m, 1H), |
| | | | | 7.12 (d, J = 8.0 Hz, 2H), 7.00-6.96 (m, |
| | | | | 3H), 6.84 (br d, J = 8.8 Hz, 2H), 6.22 (s, |
| | | | | 1H), 5.39 (br s, 1H), 5.09 (d, J = 3.6 Hz, |
| | | | | 1H), 4.93 (s, 2H), 4.40-4.26 (m, 5H), |
| | | | | 4.12-4.05 (m, 1H), 3.78 (br dd, J = 4.4, |
| | | | | 10.4 Hz, 1H), 3.73 (d, J = 9.6 Hz, 1H), |
| | | | | 3.61-3.49 (m, 1H), 3.45-3.37 (m, 5H), |
| | | | | 3.20 (br d, J = 3.2 Hz, 4H), 2.91-2.71 |
| | | | | (m, 2H), 2.43-2.40 (m, 6H), 2.27 (br d, |
| | | | | J = 2.4 Hz, 6H), 2.20 (s, 3H), 2.12-2.10 |
| | | | | (m, 1H), 2.09-2.01 (m, 2H), 1.99 (br s, |
| | | | | 3H), 1.95-1.87 (m, 2H), 1.80-1.74 (m, |
| | | | | 1H), 1.43 (br t, J = 6.0 Hz, 2H), 1.23 (s, |
| | | | | 1H), 0.97 (s, 6H), 0.92 (br d, J = 6.4 Hz, |
| | | | | 3H), 0.77-0.73 (m, 3H) |
| I- | TC | TA | 1658.7 | 8.96 (d, J = 2.4 Hz, 1H), 8.48-8.43 (m, |
| 227^{h} | | | | 1H), 8.12 (br d, J = 6.4 Hz, 1H), 7.97- |
| | | | | 7.93 (m, 1H), 7.71 (br d, J = 8.4 Hz, 2H), |
| | | | | 7.50-7.47 (m, 2H), 7.38 (dd, J = 2.8, 8.4 |
| | | | | Hz, 4H), 7.33 (s, 1H), 7.31 (s, 1H), 7.26 |
| | | | | (br t, J = 7.6 Hz, 3H), 7.18 (br d, J = 7.2 |
| | | | | Hz, 1H), 7.12 (br d, J = 8.4 Hz, 2H), 6.99 |
| | | | | (br dd, J = 4.4, 10.4 Hz, 1H), 6.85 (br d, |
| | | | | J = 8.0 Hz, 3H), 5.24 (q, J = 7.2 Hz, 1H), |
| | | | | 5.14 (d, J = 3.6 Hz, 1H), 4.56 (d, J = 9.6 |
| | | | | Hz, 1H), 4.46 (t, J = 8.0 Hz, 1H), 4.36- |
| | | | | 4.32 (m, 1H), 4.12-4.04 (m, 1H), 3.64- |
| | | | | 3.59 (m, 1H), 3.55 (br d, J = 10.4 Hz, |
| | | | | 1H), 3.51-3.45 (m, 1H), 3.24-3.20 (m, |
| | | | | 4H), 2.91 (s, 2H), 2.87-2.82 (m, 1H), |
| | | | | 2.80-2.75 (m, 4H), 2.75-2.72 (m, 1H), |
| | | | | 2.54 (br s, 3H), 2.52 (br s, 2H), 2.52 (br |
| | | | | s, 4H), 2.43 (s, 3H), 2.22 (br d, J = 4.4 |
| | | | | Hz, 5H), 1.99 (br s, 6H), 1.95-1.89 (m, |
| | | | | 2H), 1.46-1.40 (m, 6H), 1.37 (br d, J = |
| | | | | 9.6 Hz, 2H), 1.35-1.30 (m, 2H), 1.24- |
| | | | | 1.15 (m, 6H), 0.97 (s, 6H), 0.93 (br s, |
| | | | | 1H), 0.90 (s, 9H) |
| I- | TC | TA | 1658.7 | 8.98-8.96 (m, 1H), 8.54-8.45 (m, 1H), |
| 228^{h} | | | | 8.15-8.07 (m, 1H), 7.97-7.91 (m, 1H), |
| | | | | 7.70 (br d, J = 8.4 Hz, 2H), 7.39 (s, 6H), |
| | | | | 7.36 (br s, 1H), 7.35-7.33 (m, 1H), 7.33- |
| | | | | 7.32 (m, 1H), 7.29-7.24 (m, 3H), 7.21- |
| | | | | 7.17 (m, 1H), 7.13-7.09 (m, 2H), 6.92- |
| | | | | 6.78 (m, 3H), 5.23-5.17 (m, 1H), 5.13 |
| | | | | (br d, J = 2.4 Hz, 1H), 4.60-4.55 (m, |
| | | | | 1H), 4.46-4.39 (m, 1H), 4.30-4.25 (m, |
| | | | | 1H), 4.12-3.95 (m, 1H), 3.64-3.50 (m, |
| | | | | 3H), 3.23-3.15 (m, 6H), 3.01-2.94 (m, |
| | | | | 1H), 2.96 (br d, J = 6.4 Hz, 1H), 2.90 (br |
| | | | | dd, J = 2.0, 3.0 Hz, 1H), 2.86 (s, 2H), |
| | | | | 2.84-2.79 (m, 2H), 2.74 (s, 2H), 2.71 (br |
| | | | | d, J = 12.4 Hz, 2H), 2.63 (br d, J = 1.2 |
| | | | | Hz, 1H), 2.45-2.43 (m, 5H), 2.41-2.40 |
| | | | | (m, 1H), 2.27-2.17 (m, 10H), 2.01-1.97 |
| | | | | (m, 4H), 1.80-1.72 (m, 2H), 1.47-1.39 |
| | | | | (m, 6H), 1.37 (br s, 1H), 1.34-1.31 (m, |
| | | | | 2H), 1.24-1.20 (m, 6H), 1.14 (br s, 3H), |
| | | | | 0.96 (s, 9H), 0.91-0.82 (m, 2H) |
| I- | A | UL | 1540.5 | 8.98 (s, 1H), 8.39-8.33 (m, 1H), 8.10- |
| 245^{c} | | | | 7.98 (m, 1H), 7.87-7.80 (m, 1H), 7.80- |
| | | | | 7.75 (m, 1H), 7.58-7.52 (m, 1H), 7.50- |
| | | | | 7.39 (m, 4H), 7.39-7.31 (m, 5H), 7.15- |
| | | | | 7.10 (m, 1H), 7.05 (d, J = 8.4 Hz, 3H), |
| | | | | 7.01-6.96 (m, 1H), 6.69-6.65 (m, 1H), |
| | | | | 6.65-6.58 (m, 1H), 6.21 (s, 1H), 5.09 (d, |
| | | | | J = 3.6 Hz, 1H), 4.94-4.88 (m, 1H), 4.51 |
| | | | | (d, J = 9.6 Hz, 1H), 4.49 (s, 2H), 4.42 (t, |
| | | | | J = 8.0 Hz, 1H), 4.28 (br s, 1H), 3.60 (br |
| | | | | s, 2H), 3.45 (br s, 2H), 3.42-3.35 (m, |
| | | | | 4H), 3.09-3.05 (m, 4H), 2.75 (br s, 2H), |
| | | | | 2.45 (s, 3H), 2.39 (br d, J = 2.0 Hz, 4H), |
| | | | | 2.34 (br dd, J = 1.6, 6.4 Hz, 2H), 2.29- |
| | | | | 2.23 (m, 4H), 2.22 (br s, 2H), 2.20 (br s, |
| | | | | 2H), 2.17-2.16 (m, 1H), 2.09 (br s, 1H), |
| | | | | 1.97 (br s, 2H), 1.83-1.77 (m, 1H), 1.76- |
| | | | | 1.70 (m, 2H), 1.55-1.41 (m, 6H), 1.39 |
| | | | | (br s, 2H), 1.37 (d, J = 6.4 Hz, 3H), |
| | | | | 1.28-1.22 (m, 2H), 0.93 (s, 15H) |
| I- | A | UN | 1524.5 | 11.54 (s, 1H), 9.00-8.95 (m, 1H), 8.37 |
| 246ⁱ | | | | (d, J = 7.6 Hz, 1H), 8.15 (s, 1H), 7.97 (d, |
| | | | | J = 2.0 Hz, 1H), 7.93 (d, J = 2.4 Hz, 1H), |
| | | | | 7.79 (d, J = 9.4 Hz, 1H), 7.70 (dd, J = 1.6, |
| | | | | 9.2 Hz, 1H), 7.57 (d, J = 8.4 Hz, 1H), |
| | | | | 7.46-7.40 (m, 5H), 7.39-7.35 (m, 2H), |
| | | | | 7.28 (d, J = 2.4 Hz, 1H), 7.16 (d, J = 8.4 |
| | | | | Hz, 2H), 7.01-6.93 (m, 1H), 6.70 (d, J = |
| | | | | 9.2 Hz, 1H), 6.62 (d, J = 8.8 Hz, 1H), |
| | | | | 6.30 (dd, J = 2.0, 3.2 Hz, 1H), 6.25 (d, |
| | | | | J = 1.6 Hz, 1H), 5.20-5.00 (m, 1H), 4.96- |
| | | | | 4.86 (m, 1H), 4.51 (d, J = 9.2 Hz, 1H), |
| | | | | 4.42 (t, J = 8.0 Hz, 1H), 4.27 (s, 1H), 3.69 |
| | | | | (s, 2H), 3.60 (s, 2H), 3.42 (s, 4H), 3.28 |
| | | | | (s, 3H), 3.22 (s, 2H), 2.99 (s, 4H), 2.79 |
| | | | | (s, 2H), 2.45 (s, 3H), 2.40 (s, 2H), 2.36- |
| | | | | 2.30 (m, 4H), 2.29-2.20 (m, 9H), 2.12 |
| | | | | (dd, J = 6.4, 14.0 Hz, 1H), 2.04-1.97 (m, |
| | | | | 1H), 1.79 (ddd, J = 4.4, 8.4, 12.8 Hz, |
| | | | | 1H), 1.73-1.65 (m, 2H), 1.51-1.43 (m, |
| | | | | 4H), 1.37 (d, J = 7.2 Hz, 3H), 1.28-1.20 |
| | | | | (m, 2H), 0.93 (s, 9H) |
| I- | VX | KT | 1563.6 | 11.68 (s, 1H), 8.98 (s, 1H), 8.37 (d, J = |
| 248 | | | | 8.0 Hz, 1H), 8.13 (s, 1H), 8.02 (d, J = 2.4 |
| | | | | Hz, 1H), 7.90 (d, J = 9.2 Hz, 1H), 7.79 |
| | | | | (d, J = 9.2 Hz, 1H), 7.52-7.46 (m, 3H), |
| | | | | 7.45-7.41 (m, 2H), 7.40-7.32 (m, 5H), |
| | | | | 7.06 (d, J = 8.4 Hz, 2H), 7.00 (d, J = 9.2 |
| | | | | Hz, 1H), 6.66 (d, J = 9.2 Hz, 1H), 6.39 |
| | | | | (s, 1H), 6.18 (s, 1H), 5.10 (d, J = 3.6 Hz, |
| | | | | 1H), 4.97-4.86 (m, 1H), 4.51 (d, J = 9.6 |
| | | | | Hz, 1H), 4.42 (t, J = 8.0 Hz, 1H), 4.28 (s, |
| | | | | 1H), 3.63-3.55 (m, 2H), 3.44 (s, 4H), |
| | | | | 3.04 (s, 4H), 2.74-2.66 (m, 2H), 2.45 (s, |
| | | | | 3H), 2.43-2.31 (m, 6H), 2.28-2.21 (m, |
| | | | | 6H), 2.20-2.06 (m, 8H), 2.04-1.97 (m, |
| | | | | 1H), 1.89-1.69 (m, 10H), 1.61 (t, J = 6.0 |
| | | | | Hz, 2H), 1.51-1.44 (m, 4H), 1.37 (d, J = |
| | | | | 7.2 Hz, 3H), 1.28-1.21 (m, 2H), 0.93 (s, |
| | | | | 9H) |
| I- | VZ | QA | 1508.6 | 11.69-11.63 (m, 1H), 8.37-8.25 (m, |
| 249 | | | | 1H), 8.16-8.08 (m, 1H), 8.03-7.97 (m, |
| | | | | 1H), 7.92-7.85 (m, 1H), 7.83-7.75 (m, |
| | | | | 1H), 7.51-7.45 (m, 3H), 7.36-7.29 (m, |
| | | | | 5H), 7.29-7.22 (m, 3H), 7.05-7.02 (m, |
| | | | | 2H), 7.00-6.93 (m, 1H), 6.66 (dd, J = |
| | | | | 2.0, 9.0 Hz, 1H), 6.38 (d, J = 1.8 Hz, 1H), |
| | | | | 6.19 (s, 1H), 5.10 (d, J = 3.6 Hz, 1H), |
| | | | | 4.84-4.78 (m, 1H), 4.74-4.68 (m, 1H), |
| | | | | 4.54-4.47 (m, 1H), 4.43 (t, J = 8.0 Hz, |
| | | | | 1H), 4.31-4.23 (m, 1H), 3.61 (s, 2H), |
| | | | | 3.58-3.49 (m, 3H), 3.44 (s, 5H), 3.05 (s, |
| | | | | 4H), 2.73 (s, 2H), 2.38 (br s, 4H), 2.28- |
| | | | | 2.21 (m, 4H), 2.20-2.10 (m, 8H), 2.08- |
| | | | | 2.06 (m, 1H), 2.02 (s, 3H), 1.96-1.94 |
| | | | | (m, 2H), 1.82-1.76 (m, 1H), 1.75-1.69 |
| | | | | (m, 2H), 1.49-1.42 (m, 4H), 1.38 (t, J = |
| | | | | 6.4 Hz, 2H), 1.26-1.21 (m, 2H), 0.92 (s, |
| | | | | 15H) |
| I- | A | US | 1564.6 | 8.99 (s, 1H), 8.38 (br d, J = 8.0 Hz, 1H), |
| 251 | | | | 8.06 (d, J = 1.6 Hz, 1H), 7.90 (br d, J = |
| | | | | 8.8 Hz, 1H), 7.83 (br d, J = 9.2 Hz, 1H), |
| | | | | 7.67 (br d, J = 8.8 Hz, 2H), 7.59 (br d, |
| | | | | J = 7.2 Hz, 1H), 7.50 (d, J = 8.4 Hz, 2H), |
| | | | | 7.46-7.42 (m, 3H), 7.41-7.33 (m, 7H), |
| | | | | 7.32-7.28 (m, 3H), 7.22 (br d, J = 7.2 |
| | | | | Hz, 1H), 7.14 (d, J = 7.2 Hz, 1H), 6.82 |
| | | | | (br d, J = 9.2 Hz, 1H), 6.72 (br d, J = 8.8 |
| | | | | Hz, 3H), 5.23 (d, J = 4.4 Hz, 1H), 5.12 |
| | | | | (d, J = 3.2 Hz, 1H), 4.92 (br t, J = 7.2 Hz, |
| | | | | 1H), 4.56-4.50 (m, 1H), 4.42 (t, J = 8.0 |
| | | | | Hz, 1H), 4.30 (br d, J = 4.4 Hz, 2H), 4.02- |
| | | | | 3.93 (m, 1H), 3.70 (br d, J = 1.2 Hz, |
| | | | | 1H), 3.63-3.52 (m, 3H), 3.30 (br s, 5H), |
| | | | | 2.99-2.82 (m, 3H), 2.55 (s, 2H), 2.46 (s, |
| | | | | 4H), 2.42 (br s, 2H), 2.34-2.22 (m, 4H), |
| | | | | 2.15 (br d, J = 6.8 Hz, 2H), 2.08 (s, 2H), |
| | | | | 1.91 (br d, J = 12.0 Hz, 2H), 1.80 (br s, |
| | | | | 1H), 1.74-1.68 (m, 1H), 1.60-1.45 (m, |
| | | | | 9H), 1.38 (br d, J = 7.2 Hz, 4H), 1.29- |
| | | | | 1.20 (m, 3H), 1.15-1.09 (m, 1H), 1.03- |
| | | | | 0.98 (m, 1H), 0.94 (s, 9H), 0.88-0.83 |
| | | | | (m, 1H) |
| I- | WB | MK | 1550.5 | 8.98 (s, 1H), 8.37-8.35 (d, J = 8.0 Hz, |
| 252 | | | | 1H), 8.09 (s, 1H), 7.81 (s, 1H), 7.67 (s, |
| | | | | 1H), 7.65 (s, 2H), 7.49-7.47 (d, J = 8.0 |
| | | | | Hz, 1H), 7.44 (s, 2H), 7.42-7.38 (m, 3H), |
| | | | | 7.35-7.33 (m, 10H), 7.32 (br d, J = 8.4 |
| | | | | Hz, 1H), 7.14 (br d, J = 7.6 Hz, 1H), 6.75 |
| | | | | (d, J = 8.0 Hz, 1H), 6.72 (d, J = 7.6 Hz, |
| | | | | 1H), 5.50 (s, 1H), 5.22 (s, 1H), 5.21-5.09 |
| | | | | (m, 1H), 4.91 (s, 1H), 4.52-4.29 (m, 4H), |
| | | | | 4.08 (m, 1H), 3.77-3.48 (m, 10H), 2.61- |
| | | | | 2.58 (br s, 4H), 2.45(s, 3H), 2.07-2.0 (m, |
| | | | | 8 H), 1.90-1.78 (m, 4 H), 1.47-1.36 (m, |
| | | | | 11H), 0.98-0.93 (m, 3H), 0.85 (s, 9H), |
| | | | | 0.81 (br d, J = 7.5 Hz, 1H) |
| I- | UU | SR | 1581.7 | 11.57 (br s, 1H), 8.36 (d, J = 8.4 Hz, 1H), |
| 254 | | | | 8.09-7.91 (m, 2H), 7.81 (br d, J = 8.8 |
| | | | | Hz, 2H), 7.56 (br d, J = 8.8 Hz, 1H), 7.51- |
| | | | | 7.18 (m, 10H), 7.09 (br d, J = 8.4 Hz, |
| | | | | 2H), 6.91-6.80 (m, 1H), 6.66-6.61 (m, |
| | | | | 1H), 6.34 (br s, 1H), 6.23 (s, 1H), 5.10 |
| | | | | (d, J = 3.6 Hz, 1H), 4.84 (br d, J = 7.6 |
| | | | | Hz, 1H), 4.77-4.72 (m, 1H), 4.54-4.49 |
| | | | | (m, 1H), 4.45 (s, 1H), 4.27 (br d, J = 2.0 |
| | | | | Hz, 1H), 4.14 (s, 1H), 3.61 (br d, J = 0.8 |
| | | | | Hz, 4H), 3.59-3.54 (m, 2H), 3.40 (br d, |
| | | | | J = 3.6 Hz, 4H), 3.38-3.36 (m, 2H), 3.01 |
| | | | | (br s, 5H), 2.94-2.84 (m, 2H), 2.69 (br |
| | | | | s, 3H), 2.62-2.58 (m, 1H), 2.47-2.45 |
| | | | | (m, 2H), 2.41 (br s, 3H), 2.24 (br d, J = |
| | | | | 7.2 Hz, 4H), 2.18 (br s, 8H), 2.13-2.07 |
| | | | | (m, 2H), 2.05-1.96 (m, 2H), 1.90-1.82 |
| | | | | (m, 3H), 1.78 (br s, 1H), 1.56-1.51 (m, |
| | | | | 1H), 1.44 (br d, J = 6.0 Hz, 5H), 1.24 (br |
| | | | | s, 4H), 0.93 (s, 9H), 0.90 (s, 3H) |
| I- | RY | UV | 748.3 | 11.54 (s, 1H), 8.33 (d, J = 8.4 Hz, 1H), |
| 255^{b} | | | | 8.14 (s, 1H), 7.98 (d, J = 1.6 Hz, 1H), |
| | | | | 7.93 (d, J = 2.8 Hz, 1H), 7.82-7.74 (m, |
| | | | | 2H), 7.56 (d, J = 8.8 Hz, 1H), 7.45-7.41 |
| | | | | (m, 2H), 7.40 (s, 1H), 7.34 (d, J = 8.4 Hz, |
| | | | | 2H), 7.30 (d, J = 8.0 Hz, 3H), 7.05 (d, J = |
| | | | | 8.4 Hz, 2H), 7.01 (s, 1H), 6.77 (d, J = |
| | | | | 9.2 Hz, 1H), 6.61 (dd, J = 1.6, 8.8 Hz, |
| | | | | 1H), 6.33-6.29 (m, 1H), 6.24 (d, J = 1.6 |
| | | | | Hz, 1H), 5.18-5.01 (m, 1H), 4.86-4.72 |
| | | | | (m, 2H), 4.51 (d, J = 9.2 Hz, 1H), 4.43 (t, |
| | | | | J = 8.0 Hz, 1H), 4.27 (s, 1H), 4.13 (s, |
| | | | | 1H), 3.63-3.53 (m, 5H), 3.07 (s, 4H), |
| | | | | 3.00 (s, 5H), 2.86-2.79 (m, 4H), 2.73 (s, |
| | | | | 4H), 2.59 (t, J = 6.4 Hz, 2H), 2.24-2.08 |
| | | | | (m, 9H), 1.96 (s, 2H), 1.85 (s, 2H), 1.81- |
| | | | | 1.74 (m, 1H), 1.73-1.67 (m, 2H), 1.50 |
| | | | | (d, J = 6.0 Hz, 4H), 1.38 (t, J = 6.4 Hz, |
| | | | | 2H), 1.25 (s, 4H), 0.93 (s, 15H) |
| I- | NV | WE | 1602.6 | 11.69-11.56 (m, 1H), 8.37 (s, 1H), 8.11 |
| 260 | | | | (s, 1H), 8.02-7.94 (m, 1H), 7.88-7.76 |
| | | | | (m, 2H), 7.55-7.41 (m, 3H), 7.38-7.34 |
| | | | | (m, 3H), 7.34-7.19 (m, 5H), 7.08 (d, J = |
| | | | | 8.4 Hz, 2H), 6.98-6.86 (m, 1H), 6.67- |
| | | | | 6.62 (m, 1H), 6.40-6.32 (m, 1H), 6.23- |
| | | | | 6.18 (m, 1H), 5.12-5.07 (m, 1H), 4.85- |
| | | | | 4.78 (m, 1H), 4.76-4.71 (m, 1H), 4.54- |
| | | | | 4.48 (m, 1H), 4.46-4.40 (m, 1H), 4.30- |
| | | | | 4.23 (m, 1H), 3.64-3.54 (m, 8H), 3.02 |
| | | | | (d, J = 1.6 Hz, 6H), 2.70-2.66 (m, 2H), |
| | | | | 2.42-2.35 (m, 8H), 2.26-2.22 (m, 4H), |
| | | | | 2.18 (s, 6H), 2.11-2.06 (m, 2H), 2.05- |
| | | | | 2.01 (m, 1H), 1.98 (dd, J = 2.0, 8.8 Hz, |
| | | | | 1H), 1.95-1.81 (m, 2H), 1.80-1.73 (m, |
| | | | | 2H), 1.73-1.64 (m, 2H), 1.58-1.36 (m, |
| | | | | 8H), 1.27-1.19 (m, 6H), 0.92 (s, 9H), |
| | | | | 0.89 (s, 3H) |
| I- | RY | VD | 1510.6 | 11.72-11.38 (m, 1H), 8.40-8.25 (m, |
| 262 | | | | 1H), 8.15 (s, 1H), 8.05-7.99 (m, 1H), |
| | | | | 7.96 (d, J = 2.4 Hz, 1H), 7.85-7.74 (m, |
| | | | | 2H), 7.55 (d, J = 8.8 Hz, 1H), 7.45 (t, J = |
| | | | | 2.8 Hz, 1H), 7.42 (d, J = 8.0 Hz, 2H), |
| | | | | 7.36 (d, J = 8.4 Hz, 3H), 7.31 (d, J = 8.0 |
| | | | | Hz, 2H), 7.09 (d, J = 8.4 Hz, 2H), 6.86- |
| | | | | 6.75 (m, 1H), 6.64 (d, J = 8.4 Hz, 1H), |
| | | | | 6.36-6.27 (m, 1H), 6.23 (s, 1H), 5.18- |
| | | | | 5.01 (m, 1H), 4.86-4.73 (m, 2H), 4.52 |
| | | | | (d, J = 9.2 Hz, 1H), 4.45 (t, J = 8.0 Hz, |
| | | | | 1H), 4.27 (s, 1H), 4.13 (s, 1H), 3.61 (s, |
| | | | | 3H), 3.60-3.55 (m, 6H), 3.01 (s, 4H), |
| | | | | 2.70 (s, 3H), 2.55 (s, 1H), 2.45-2.37 (m, |
| | | | | 4H), 2.37-2.32 (m, 6H), 2.30-2.24 (m, |
| | | | | 2H), 2.23-2.15 (m, 6H), 2.14-2.06 (m, |
| | | | | 2H), 2.06-1.96 (m, 2H), 1.95-1.88 (m, |
| | | | | 1H), 1.82-1.75 (m, 1H), 1.70 (t, J = 6.8 |
| | | | | Hz, 2H), 1.56-1.41 (m, 6H), 1.35-1.15 |
| | | | | (m, 5H), 0.97-0.90 (m, 12H) |
| I- | LB | VG | 1522.6 | 11.68 (br s, 1H), 8.34 (d, J = 8.0 Hz, 1H), |
| 264 | | | | 8.14 (br s, 1H), 8.03 (s, 1H), 7.90 (br s, |
| | | | | 1H), 7.79 (d, J = 9.2 Hz, 1H), 7.53-7.47 |
| | | | | (m, 3H), 7.44-7.26 (m, 8H), 7.05 (d, J = |
| | | | | 8.4 Hz, 3H), 6.67 (br d, J = 8.4 Hz, 1H), |
| | | | | 6.40 (br s, 1H), 6.19 (s, 1H), 5.10 (d, J = |
| | | | | 3.6 Hz, 1H), 4.84 (br d, J = 7.6 Hz, 1H), |
| | | | | 4.74 (t, J = 5.6 Hz, 1H), 4.52 (d, J = 9.2 |
| | | | | Hz, 1H), 4.45 (s, 1H), 4.27 (br d, J = 2.8 |
| | | | | Hz, 1H), 4.13 (s, 1H), 3.61 (br s, 2H), |
| | | | | 3.56 (br d, J = 6.8 Hz, 2H), 3.44 (br d, |
| | | | | J = 2.4 Hz, 4H), 3.40-3.37 (m, 3H), 3.06 |
| | | | | (br s, 4H), 2.78-2.72 (m, 2H), 2.43- |
| | | | | 2.36 (m, 4H), 2.27 (br t, J = 7.2 Hz, 3H), |
| | | | | 2.24-2.22 (m, 1H), 2.20-2.13 (m, 6H), |
| | | | | 2.11 (br s, 1H), 2.03-1.99 (m, 1H), 1.96 |
| | | | | (br s, 2H), 1.84-1.78 (m, 1H), 1.74 (br |
| | | | | d, J = 7.6 Hz, 2H), 1.50-1.43 (m, 4H), |
| | | | | 1.39 (br s, 2H), 1.25 (br s, 6H), 0.93 (s, |
| | | | | 15H) |
| I- | NV | VJ | 1607.7 | 11.73-11.64 (m, 1H), 8.44 (d, J = 8.0 |
| 265 | | | | Hz, 1H), 8.17-8.11 (m, 1H), 8.06-7.98 |
| | | | | (m, 1H), 7.92-7.86 (m, 1H), 7.83 (d, J = |
| | | | | 9.2 Hz, 1H), 7.56-7.47 (m, 3H), 7.42 (d, |
| | | | | J = 8.0 Hz, 2H), 7.33 (dd, J = 8.0 14.7 |
| | | | | Hz, 5H), 7.12-7.05 (m, 2H), 7.03-6.96 |
| | | | | (m, 1H), 6.74-6.64 (m, 1H), 6.44-6.36 |
| | | | | (m, 1H), 6.24-6.17 (m, 1H), 5.13-5.08 |
| | | | | (m, 1H), 5.01-4.93 (m, 1H), 4.52 (d, J = |
| | | | | 9.4 Hz, 1H), 4.46 (t, J = 8.0 Hz, 1H), |
| | | | | 4.30-4.21 (m, 1H), 4.19-4.11 (m, 1H), 3.67- |
| | | | | 3.52 (m, 8H), 3.46 (dd, J = 6.0, 10.0 Hz, |
| | | | | 2H), 3.23 (s, 2H), 3.04 (s, 4H), 2.82- |
| | | | | 2.60 (m, 3H), 2.56-2.53 (m, 3H), 2.46- |
| | | | | 2.40 (m, 8H), 2.28-2.22 (m, 4H), 2.19 |
| | | | | (s, 8H), 2.12-2.07 (m, 2H), 2.05-1.95 |
| | | | | (m, 2H), 1.88-1.82 (m, 1H), 1.78-1.70 |
| | | | | (m, 3H), 1.55-1.36 (m, 7H), 1.25 (d, J = |
| | | | | 3.2 Hz, 5H), 0.91 (s, 9H), 0.90 (s, 3H) |
| I- | LB | VJ | 1522.6 | 11.66 (s, 1H), 8.45-8.39 (m, 1H), 8.14- |
| 266 | | | | 8.11 (m, 1H), 8.02 (d, J = 2.4 Hz, 1H), |
| | | | | 7.89 (d, J = 8.4 Hz, 1H), 7.81 (d, J = 9.2 |
| | | | | Hz, 1H), 7.49 (dd, J = 2.8, 5.6 Hz, 3H), |
| | | | | 7.41 (d, J = 8.0 Hz, 2H), 7.37-7.28 (m, |
| | | | | 5H), 7.04 (d, J = 8.4 Hz, 2H), 6.98 (d, |
| | | | | J = 8.8 Hz, 1H), 6.72-6.61 (m, 1H), |
| | | | | 6.43-6.35 (m, 1H), 6.24-6.17 (m, 1H), 5.11 |
| | | | | (d, J = 3.2 Hz, 1H), 5.00-4.93 (m, 1H), |
| | | | | 4.54-4.44 (m, 2H), 4.29-4.23 (m, 1H), |
| | | | | 4.13 (s, 1H), 3.59 (s, 3H), 3.55 (d, J = 6.4 |
| | | | | Hz, 2H), 3.52 (s, 2H), 3.25-3.22 (m, |
| | | | | 4H), 3.05 (s, 4H), 2.74 (s, 2H), 2.40 (s, |
| | | | | 4H), 2.35 (s, 2H), 2.31-2.22 (m, 4H), |
| | | | | 2.22-2.05 (m, 8H), 1.99 (s, 1H), 1.95 (s, |
| | | | | 2H), 1.80-1.70 (m, 3H), 1.52-1.43 (m, |
| | | | | 4H), 1.38 (t, J = 6.4 Hz, 2H), 1.25 (d, J = |
| | | | | 3.2 Hz, 5H), 0.93 (d, J = 3.6 Hz, 15H) |
| I- | VK | VL | 1593.7 | 11.67 (s, 1H), 9.77 (s, 1H), 8.24 (d, J = |
| 267 | | | | 8.0 Hz, 1H), 8.13 (d, J = 3.6 Hz, 1H), |
| | | | | 8.02 (d, J = 2.4 Hz, 1H), 7.89 (d, J = 8.8 |
| | | | | Hz, 1H), 7.77 (d, J = 9.2 Hz, 1H), 7.54- |
| | | | | 7.45 (m, 3H), 7.34 (d, J = 8.4 Hz, 3H), |
| | | | | 7.13 (d, J = 8.0 Hz, 1H), 7.08 (d, J = 8.4 |
| | | | | Hz, 2H), 7.03-6.95 (m, 1H), 6.89-6.85 |
| | | | | (m, 1H), 6.84 (s, 1H), 6.66 (d, J = 8.0 Hz, |
| | | | | 1H), 6.39 (dd, J = 1.6, 3.2 Hz, 1H), 6.18 |
| | | | | (s, 1H), 5.12-5.03 (m, 2H), 4.54-4.47 |
| | | | | (m, 1H), 4.42 (t, J = 8.0 Hz, 1H), 4.26 (s, |
| | | | | 1H), 4.04 (s, 1H), 3.59 (d, J = 3.2 Hz, |
| | | | | 6H), 3.38 (d, J = 6.4 Hz, 4H), 3.09-2.99 |
| | | | | (m, 4H), 2.68 (dd, J = 1.6, 10.0 Hz, 2H), |
| | | | | 2.42 (s, 9H), 2.30-2.12 (m, 12H), 2.12- |
| | | | | 2.05 (m, 2H), 2.05-1.94 (m, 2H), 1.88- |
| | | | | 1.71 (m, 4H), 1.57-1.34 (m, 7H), 1.26 |
| | | | | (d, J = 7.2 Hz, 8H), 0.93 (s, 9H), 0.89 |
| | | | | (s, 3H) |
| I- | NV | WG | 1637.2 | 11.67 (s, 1H), 8.32-8.30 (br d, J = 8.0 Hz, |
| 268 | | | | 1H), 8.12 (s, 1H), 8.11 (s, 1H), 8.02-8.01 |
| | | | | (d, J = 4.0 Hz, 1H), 8.01 (s, 1H), 7.50- |
| | | | | 7.06 (m, 3H), 6.39-6.38 (br d, J = 4.0 Hz, |
| | | | | 1H), 6.37 (s, 1H), 6.18 (s, 1H), 5.08-5.07 |
| | | | | (br d, J = 4.0 Hz, 2H), 4.39-4.20(m, 3H), |
| | | | | 3.97 (s, 3H), 3.81 (s, 3H), 3.80 (m, 6H), |
| | | | | 3.60-3.58 (m, 6H), 3.03 (m, 4H), 2.44- |
| | | | | 2.17 (m, 22H), 6.75 (d, J = 8.0 Hz, 1H), |
| | | | | 6.72 (d, J = 7.6 Hz, 1H), 5.50 (s, 1H), |
| | | | | 5.22 (s, 1H), 5.21-5.09 (m, 1H), 4.91 (s, |
| | | | | 1H), 4.52-4.29 (m, 4H), 4.08(m, 1H), |
| | | | | 3.77-3.48 (m, 10H), 2.61-2.58 (br s, 4H), |
| | | | | 2.45 (s, 3H), 2.07-2.0 (m, 8 H), 1.47-1.23 |
| | | | | (m, 16H), 0.93-0.89 (m, 17H) |
| I- | NV | VS | 811.7 | 11.67 (s, 1H), 8.15-8.08 (m, 2H), 8.02 |
| 270^{b} | | | | (d, J = 2.4 Hz, 1H), 7.89 (d, J = 8.8 Hz, |
| | | | | 1H), 7.76 (d, J = 9. 2 Hz, 1H), 7.54-7.45 |
| | | | | (m, 3H), 7.41-7.37 (m, 2H), 7.34 (d, J = |
| | | | | 7.2 Hz, 5H), 7.08 (d, J = 8.4 Hz, 2H), |
| | | | | 7.02-6.94 (m, 1H), 6.66 (d, J = 7.6 Hz, |
| | | | | 1H), 6.41-6.35 (m, 1H), 6.19 (s, 1H), |
| | | | | 5.10 (d, J = 3.6 Hz, 1H), 4.68 (d, J = 8.8 |
| | | | | Hz, 1H), 4.59-4.53 (m, 2H), 4.48 (d, J = |
| | | | | 9.2 Hz, 1H), 4.30 (d, J = 3.6 Hz, 1H), |
| | | | | 4.10 (s, 1H), 3.63-3.55 (m, 6H), 3.38 (d, |
| | | | | J = 6.0 Hz, 4H), 3.04 (s, 4H), 2.70 (s, |
| | | | | 1H), 2.47-2.39 (m, 9H), 2.21 (dd, J = |
| | | | | 7.2, 14.4 Hz, 12H), 2.09 (d, J = 7.2 Hz, |
| | | | | 2H), 2.04 (d, J = 4.4 Hz, 1H), 2.00-1.93 |
| | | | | (m, 2H), 1.85 (d, J = 18.0 Hz, 1H), 1.72 |
| | | | | (t, J = 6.8 Hz, 2H), 1.59-1.48 (m, 2H), |
| | | | | 1.47-1.37 (m, 5H), 1.23 (s, 5H), 1.05 (s, |
| | | | | 3H), 0.98 (s, 3H), 0.89 (s, 3H), 0.84 (s, |
| | | | | 9H) |
| I- | NV | VU | 1621.7 | 11.73-11.59 (m, 1H), 8.33 (d, J = 9.2 |
| 271 | | | | Hz, 1H), 8.13 (s, 1H), 8.10 (s, 1H), 8.01 |
| | | | | (s, 1H), 7.90-7.80 (m, 2H), 7.54-7.44 |
| | | | | (m, 3H), 7.40-7.32 (m, 6H), 7.31 (d, J = |
| | | | | 3.2 Hz, 1H), 7.08 (d, J = 8.4 Hz, 2H), |
| | | | | 7.02-6.89 (m, 1H), 6.66 (d, J = 8.8 Hz, |
| | | | | 1H), 6.42-6.34 (m, 1H), 6.23-6.15 (m, |
| | | | | 1H), 5.07 (d, J = 3.2 Hz, 1H), 4.81-4.75 |
| | | | | (m, 1H), 4.57-4.49 (m, 2H), 4.40 (s, |
| | | | | 1H), 4.26 (d, J = 2.0 Hz, 1H), 4.12 (s, |
| | | | | 1H), 3.60 (s, 6H), 3.43-3.37 (m, 4H), |
| | | | | 3.08-2.98 (m, 4H), 2.72-2.68 (m, 1H), |
| | | | | 2.41 (dd, J = 1.6, 3.2 Hz, 8H), 2.29-2.12 |
| | | | | (m, 13H), 2.11 (s, 1H), 2.06 (d, J = 18.0 |
| | | | | Hz, 2H), 1.94-1.89 (m, 1H), 1.88-1.82 |
| | | | | (m, 1H), 1.75-1.67 (m, 3H), 1.58-1.34 |
| | | | | (m, 7H), 1.31-1.16 (m, 5H), 1.09 (s, |
| | | | | 3H), 1.03 (s, 3H), 0.95-0.87 (m, 12H) |

^{a}The coupling was run from 0.5-18 hr at −10-25° C. A variety of other bases such as TEA and solvents such as DCM could be employed for the coupling as well. Final compounds were purified via standard techniques including prep-HPLC and chromatography. HOAt was also added when chiral coupling partners were employed to help suppress racemization.
^{b}LCMS data reported as (M/2 + H)⁺.
^{c}The product of the coupling was further deprotected with CsF in DMSO at 25-50° C. for 2 hr. The final compound was purified by prep-HPLC or reverse phase HPLC.
^{d}LCMS data reported as (M + 23)⁺.
^{e}LCMS data reported as (M + 3H)⁺.
^{f}The diastereomers were separated by reverse phase HPLC, where absolute stereochemistry was arbitrarily assigned.
^{g}The product of the coupling was then separated reversed-phase HPLC [column: DAICEL CHIRALPAK IG (250 mm*30 mm, 10 um); mobile phase: [ACN/MeOH(0.1% NH3H2O)]; B %: 60%-60%, 7.2; 70 min]. The absolute sterochemistry of the diastereomers were assigned arbitrarily.
^{h}The product of the coupling was further separated by SFC “Column: (Chiralcel OD-3 50 × 4.6 mm I.D., 3 um, Mobile phase: Phase A for CO2, and Phase B for MeOH + ACN(0.05% DEA); Isocratic elution: 50% B in AFlow rate: 3 mL/min; Detector: PDA; Column Temp: 35 C.; Back Pressure: 100 Bar”) then purified by prep-HPLC (TFA condition; column: Phenomenex luna C18 150*25 mm* 10 um; mobile phase: [water(FA)-ACN]; B %: 34%-64%, 15 min). The absolute stereochemistry of the diastereomers was assigned arbitrarily.
ⁱThe product of the coupling was deprotected with HCl/dioxane in DCM at rt for 1 h. The final product was purified by prep-HPLC.

### Example 3: Synthesis of Compounds of Formula I (Method 2)

### Synthesis of (2S,4R)—N-(2-(2-(4-((R)-3-((4-(N-(4-(2-((S)-2-(2-cyclopropylphenyl)pyrolidine-1-yl)-7-azaspiro[3.5]nonan-7-yl)benzoyl)sulfamoyl)-2-((trifluoromethyl)sulfonyl)phenyl)amino)-4-(phenylthio)butyl)piperazin-1-yl)ethoxy)-4 (4-methylthiazol-5-yl)benzyl)-1-((S)-2-(1-fluorocyclopropanecarboxamido)-3,3-dimethylbutanoyl)-4-hydroxypyrrolidine-2-carboxamide (I-31)

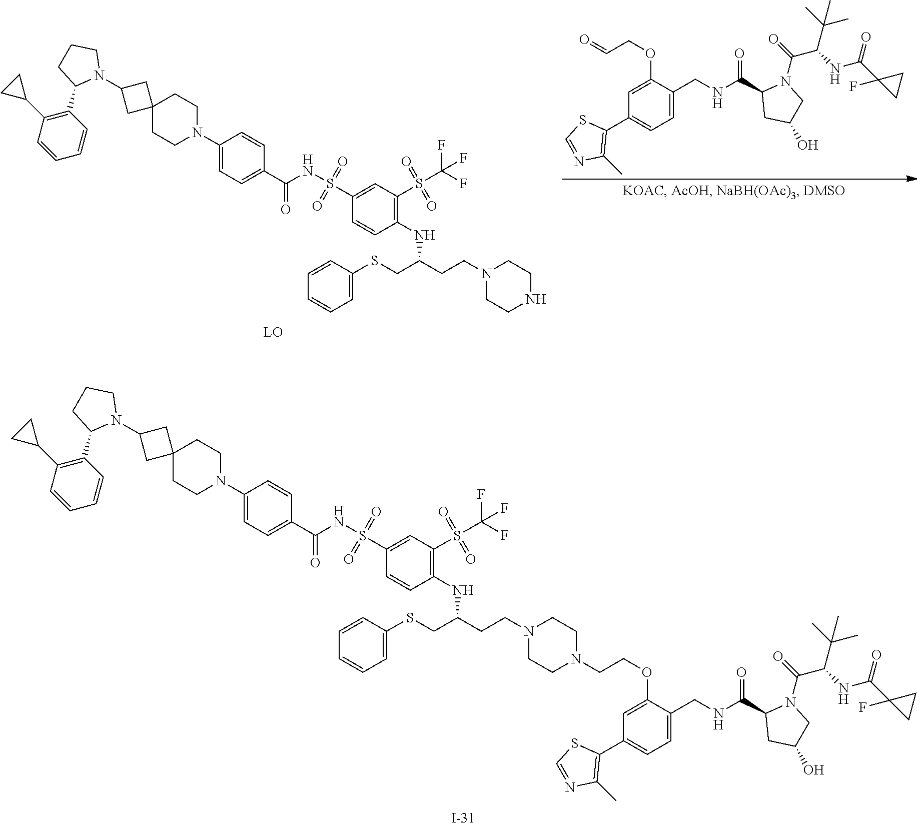

To a solution of 4-(2-((S)-2-(2-cyclopropylphenyl)pyrrolidin-1-yl)-7-azaspiro[3.5]nonan-7-yl)-N-((4-(((R)-1-(phenylthio)-4-(piperazin-1-yl)butan-2-yl)amino)-3-((trifluoromethyl)sulfonyl)phenyl)sulfonyl)benzamide (40 mg, 41 μmol, Intermediate LO) in DMSO (1 mL) was added KOAc (6.10 mg, 62.1 μmol), AcOH (7.47 mg, 124 μmol, 7.11 uL) and (2S,4R)-1-((S)-2-(1-fluorocyclopropanecarboxamido)-3,3-dimethylbutanoyl)-4-hydroxy-N-(4-(4-methylthiazol-5-yl)-2-(2-oxoethoxy)benzyl)pyrrolidine-2-carboxamide (23.8 mg, 41.4 μmol, Intermediate LM) at 0° C. Then the reaction was stirred at 0° C. for 1 hr and then NaBH(OAc) 3 (21.9 mg, 103 μmol) was added. Then the mixture was stirred at 25° C. for 1 hr. On completion, the reaction mixture was concentrated under reduced pressure to give a residue. The residue was purified by prep-HPLC (FA condition) to give the title compound (15 mg, 23% yield) as a white solid. ¹H NMR (400 MHz, DMSO-d₆) δ=8.98 (s, 1H), 8.50 (t, J=5.6 Hz, 1H), 8.13 (s, 1H), 8.05 (d, J=1.2 Hz, 1H), 7.90 (d, J=9.2 Hz, 1H), 7.68 (d, J=8.8 Hz, 2H), 7.55 (d, J=7.6 Hz, 1H), 7.41 (d, J=7.6 Hz, 1H), 7.35-7.32 (m, 2H), 7.30-7.25 (m, 4H), 7.18-7.14 (m, 2H), 7.02 (s, 1H), 6.96 (d, J=7.6 Hz, 2H), 6.85 (d, J=8.8 Hz, 1H), 6.75 (d, J=8.8 Hz, 2H), 6.71-6.67 (m, 1H), 5.17 (d, J=3.6 Hz, 1H), 4.59 (d, J=9.2 Hz, 1H), 4.52 (t, J=8.4 Hz, 1H), 4.34 (d, J=4.8 Hz, 2H), 4.28 (d, J=6.4 Hz, 1H), 4.22 (d, J=4.8 Hz, 1H), 4.18-4.14 (m, 2H), 4.02-3.97 (m, 1H), 3.68-3.57 (m, 4H), 3.13-3.06 (m, 4H), 3.00 (s, 4H), 2.84-2.75 (m, 2H), 2.54 (s, 2H), 2.45 (s, 3H), 2.12-2.03 (m. 4H), 1.95-1.88 (m, 4H), 1.76-1.68 (m, 2H), 1.63-1.56 (m, 2H), 1.52-1.31 (m, 12H), 1.26-1.18 (m. 4H), 0.94 (s, 9H), 0.69-0.55 (m, 3H). LC-MS (ESI+) m/z 1524.5 (M+H)⁺.

TABLE 4
Compounds synthesized via Method 2, using the corresponding
amines and aldehydes for the coupling.

| | | | LCMS | |
|---|---|---|---|---|
| | | | (ESI+) | |
| | | | m/z | |
| I-#^{a} | Amine | Aldehyde | (M + H)⁺ | ¹H NMR (400 MHz, DMSO-d6) |
| I-9 | LJ | LV | 1492.6 | 8.93 (s, 1 H), 8.46 (d, J = 2.0 Hz, |
| | | | | 1 H), 8.34-8.40 (m, 1 H), 8.12- |
| | | | | 8.25 (m, 1 H), 7.76-7.83 (m, 2 |
| | | | | H), 7.73 (d, J = 8.8 Hz, 2 H), 7.53 |
| | | | | (s, 1 H), 7.50 (s, 4 H), 7.42-7.45 |
| | | | | (m, 2 H), 7.36-7.40 (m, 3 H), |
| | | | | 7.30-7.33 (m, 3 H), 7.22-7.26 |
| | | | | (m, 2 H), 7.15-7.19 (m, 1 H), |
| | | | | 6.89-6.94 (m, 1 H), 6.78-6.83 |
| | | | | (m, 2 H), 5.07-5.14 (m, 1 H), |
| | | | | 4.88-4.95 (m, 1 H), 4.52 (d, J = |
| | | | | 9.2 Hz, 1 H), 4.42 (t, J = 8.0 Hz, |
| | | | | 1 H), 4.28 (s, 1 H), 4.03-4.12 (m, |
| | | | | 1 H), 3.56-3.65 (m, 4 H), 3.43 (s, |
| | | | | 3 H), 3.15 (s, 4 H), 3.11 (s, 1 H), |
| | | | | 2.90-3.03 (m, 2 H), 2.68 (s, 1 H), |
| | | | | 2.60 (s, 4 H), 2.46 (s, 4 H), 2.41 |
| | | | | (s, 6 H), 2.32-2.36 (m, 2 H), 2.20- |
| | | | | 2.28 (m, 2 H), 2.07-2.13 (m, 2 |
| | | | | H), 1.99-2.05 (m, 2 H), 1.77- |
| | | | | 1.83 (m, 1 H), 1.47-1.55 (m, 2 |
| | | | | H), 1.40-1.46 (m, 3 H), 1.38 (d, |
| | | | | J = 7.2 Hz, 4 H), 1.21-1.30 (m, |
| | | | | 8 H), 0.94 (s, 9 H) |
| I-13 | LZ | MA | 1408.5 | 8.98 (s, 1H), 8.44 (d, J = 2.0 Hz, |
| | | | | 1H), 8.36 (d, J = 7.6 Hz, 1H), 8.13 |
| | | | | (s, 1H), 7.79 (d, .J = 9.6 Hz, 2H), |
| | | | | 7.72 (d, J = 8.8 Hz, 2H), 7.53- |
| | | | | 7.49 (m, 1H), 7.47 (s, 3H), 7.45- |
| | | | | 7.41 (m, 2H), 7.37 (d, J = 8.0 Hz, |
| | | | | 3H), 7.35 (d, J = 1.6 Hz, 1H), 7.34- |
| | | | | 7.32 (m, 1H), 7.30 (s, 2H), 7.27- |
| | | | | 7.22 (m, 3H), 7.19-7.14 (m, |
| | | | | 1H), 6.94-6.89 (m, 1H), 6.79 (d, |
| | | | | J = 8.8 Hz, 2H), 5.09 (d, J = 1.6 |
| | | | | Hz, 1H), 4.96-4.87 (m, 1H), 4.54- |
| | | | | 4.49 (m, 1H), 4.44-4.38 (m, |
| | | | | 1H), 4.28 (d, J = 2.4 Hz, 1H), 4.12- |
| | | | | 4.02 (m, 1H), 3.64-3.56 (m, |
| | | | | 2H), 3.38 (s, 3H), 3.18-3.10 (m, |
| | | | | 6H), 2.45 (s, 3H), 2.42-2.37 (m, |
| | | | | 8H), 2.30-2.19 (m, 4H), 1.99 (d, |
| | | | | J = 8.8 Hz, 6H), 1.84-1.75 (m, |
| | | | | 2H), 1.56-1.42 (m, 5H), 1.37 (d, |
| | | | | J = 7.2 Hz, 3H), 1.24 (s, 5H), 0.93 |
| | | | | (s, 9H) |
| I-14 | MD | MA | 1353.5 | 8.99 (s, 1 H), 8.46 (d, J = 2.0 Hz, |
| | | | | 1 H), 8.37 (d, J = 7.6 Hz, 1 H), |
| | | | | 7.77-7.84 (m, 2 H), 7.73 (d, J = |
| | | | | 8.8 Hz, 2 H), 7.50 (s, 1 H), 7.48 |
| | | | | (s, 4 H), 7.42-7.45 (m, 2 H), 7.33- |
| | | | | 7.41 (m, 5 H), 7.30-7.33 (m, 2 |
| | | | | H), 7.22-7.27 (m, 3 H), 7.15- |
| | | | | 7.19 (m, 1 H), 6.89-6.96 (m, 1 |
| | | | | H), 6.80 (d, J = 8.8 Hz, 2 H), 5.11 |
| | | | | (d, J = 3.6 Hz, 1 H), 4.92 (t, J = |
| | | | | 7.2 Hz, 1 H), 4.52 (d, J = 9.2 Hz, |
| | | | | 1 H), 4.42 (t, J = 8.0 Hz, 1 H), |
| | | | | 4.28 (d, J = 1.6 Hz, 1 H), 4.09 (d, |
| | | | | J = 4.4 Hz, 1 H), 3.60 (d, J = 3.6 |
| | | | | Hz, 2 H), 3.39 (s, 4 H), 3.14 (s, 5 |
| | | | | H), 2.69-2.77 (m, 2 H), 2.46 (s, |
| | | | | 3 H), 2.40 (s, 5 H), 2.24 (dd, J = |
| | | | | 14.4, 6.8 Hz, 2 H), 2.11 (dt, J = |
| | | | | 13.6, 6.8 Hz, 3 H), 1.96-2.06 (m, |
| | | | | 2 H), 1.80 (td, J = 8.4, 4.0 Hz, 1 |
| | | | | H), 1.49 (dd, J = 15.6, 6.4 Hz, 4 |
| | | | | H), 1.37 (d, J = 7.2 Hz, 3 H), 1.24 |
| | | | | (s, 5 H), 0.93 (s, 9 H) |
| I-43 | LJ | MA | 1479.6 | 8.97-9.01 (m, 1 H), 8.44-8.49 |
| | | | | (m, 1 H), 8.37 (d, J = 7.6 Hz, 1 H), |
| | | | | 8.11-8.29 (m, 1 H), 7.77-7.83 |
| | | | | (m, 2 H), 7.73 (d, J = 8.8 Hz, 2 H), |
| | | | | 7.50-7.53 (m, 5 H), 7.42-7.45 |
| | | | | (m, 2 H), 7.36-7.40 (m, 3 H), |
| | | | | 7.32 (d, J = 9.6 Hz, 3 H), 7.23- |
| | | | | 7.27 (m, 2 H), 7.15-7.19 (m, 1 |
| | | | | H), 6.87-6.92 (m, 1 H), 6.78- |
| | | | | 6.82 (m, 2 H), 5.06-5.13 (m, 1 |
| | | | | H), 4.89-4.96 (m, 1 H), 4.52 (d, |
| | | | | J = 9.2 Hz, 1 H), 4.39-4.45 (m, 1 |
| | | | | H), 4.25-4.31 (m, 1 H), 4.01- |
| | | | | 4.12 (m, 1 H), 3.54-3.68 (m, 4 |
| | | | | H), 3.43 (s, 3 H), 3.08-3.19 (m, |
| | | | | 6 H), 2.77-3.01 (m, 2 H), 2.55 (s, |
| | | | | 1 H), 2.46 (s, 4 H), 2.44-2.45 (m, |
| | | | | 1 H), 2.41 (s, 7 H), 2.30-2.36 (m, |
| | | | | 3 H), 2.18-2.30 (m, 3 H), 2.07- |
| | | | | 2.15 (m, 2 H), 1.98-2.04 (m, 2 |
| | | | | H), 1.80 (ddd, J = 12.8, 8.4, 4.8 |
| | | | | Hz, 1 H), 1.46-1.52 (m, 2 H), |
| | | | | 1.41-1.45 (m, 2 H), 1.38 (d, J = |
| | | | | 6.8 Hz, 4 H), 1.16-1.33 (m, 6 H), |
| | | | | 0.94 (s, 9 H) |
| I-67 | OA | OB | 1451.5 | 11.65-11.47 (m, 1H), 9.01-8.96 |
| | | | | (m, 1H), 8.36 (d, J = 8.0 Hz, 1H), |
| | | | | 8.07-7.99 (m, 1H), 7.98-7.90 |
| | | | | (m, 1H), 7.80 (d, J = 9.2 Hz, 2H), |
| | | | | 7.54 (d, J = 7.2 Hz, 2H), 7.48- |
| | | | | 7.41 (m, 6H), 7.40-7.37 (m, 3H), |
| | | | | 7.33-7.27 (m, 4H), 7.13-7.09 |
| | | | | (m, 1H), 6.91-6.74 (m, 1H), 6.58 |
| | | | | (d, J = 9.6 Hz, 1H), 6.38-6.30 |
| | | | | (m, 1H), 6.21-6.13 (m, 1H), 5.19 |
| | | | | (d, J = 4.4 Hz, 1H), 5.11 (d, J = |
| | | | | 3.6 Hz, 1H), 4.92 (t, J = 7.2 Hz, |
| | | | | 1H), 4.52 (d, J = 9.2 Hz, 1H), 4.42 |
| | | | | (t, J = 8.4 Hz, 1H), 4.30-4.24 (m, |
| | | | | 2H), 3.63-3.54 (m, 3H), 3.44- |
| | | | | 3.36 (m, 3H), 2.46-2.45 (m, 4H), |
| | | | | 2.42-2.31 (m, 4H), 2.30-2.22 |
| | | | | (m, 2H), 2.18-2.08 (m, 2H), 2.08- |
| | | | | 1.94 (m, 2H), 1.88-1.73 (m, |
| | | | | 5H), 1.59-1.46 (m, 6H), 1.37 (d, |
| | | | | J = 7.2 Hz, 3H), 1.29-1.19 (m, |
| | | | | 3H), 1.09-1.01 (m, 1H), 0.93 (s, |
| | | | | 9H), 0.82-0.74 (m, 1H) |
| I-69 | PK | OB | 1468.6 | 11.53 (s, 1H), 8.99 (s, 1H), 8.37 |
| | | | | (d, J = 7.6 Hz, 1H), 8.20-8.15 |
| | | | | (m, 1H), 7.98 (s, 1H), 7.93 (d, J = |
| | | | | 2.4 Hz, 1H), 7.82-7.77 (m, 1H), |
| | | | | 7.74 (d, J = 8.4 Hz, 1H), 7.57 (d, |
| | | | | J = 8.8 Hz, 1H), 7.45-7.42 (m, |
| | | | | 3H), 7.39 (s, 1H), 7.38-7.33 (m, |
| | | | | 3H), 7.28 (s, 1H), 7.06 (d, J = 8.4 |
| | | | | Hz, 2H), 6.76-6.68 (m, 1H), 6.64- |
| | | | | 6.60 (m, 1H), 6.34-6.30 (m, |
| | | | | 1H), 6.25 (s, 1H), 5.26-4.98 (m, |
| | | | | 1H), 4.97-4.88 (m, 1H), 4.52 (d, |
| | | | | J = 9.2 Hz, 1H), 4.42 (t, J = 7.6 |
| | | | | Hz, 1H), 4.31-4.25 (m, 1H), 3.60 |
| | | | | (s, 2H), 3.00 (s, 5H), 2.73 (s, 2H), |
| | | | | 2.46 (s, 4H), 2.30-2.24 (m, 4H), |
| | | | | 2.23-2.14 (m, 8H), 1.96 (s, 4H), |
| | | | | 1.84-1.69 (m, 4H), 1.54-1.43 |
| | | | | (m, 5H), 1.40-1.36 (m, 5H), 1.28- |
| | | | | 1.18 (m, 3H), 0.93 (s, 15H) |
| I-74 | LB | OB | 1523.6 | 11.57 (s, 1H), 8.98 (s, 1H), 8.36 |
| | | | | (d, J = 7.6 Hz, 1H), 8.14 (s, 1H), |
| | | | | 8.02 (s, 1H), 7.95 (d, J = 2.4 Hz, |
| | | | | 1H), 7.79 (d, J = 9.2 Hz, 2H), 7.54 |
| | | | | (d, J = 8.8 Hz, 1H), 7.43 (d, J = |
| | | | | 8.4 Hz, 3H), 7.40-7.32 (m, 5H), |
| | | | | 7.04 (d, J = 8.0 Hz, 3H), 6.82 (d, |
| | | | | J = 9.2 Hz, 1H), 6.66-6.59 (m, |
| | | | | 1H), 6.33 (s, 1H), 6.23 (s, 1H), |
| | | | | 5.22-4.99 (m, 1H), 4.95-4.88 |
| | | | | (m, 1H), 4.52 (d, J = 9.2 Hz, 1H), |
| | | | | 4.42 (t, J = 8.0 Hz, 1H), 4.28 (s, |
| | | | | 1H), 3.63-3.58 (m, 2H), 3.32 (d, |
| | | | | J = 5.2 Hz, 7H), 3.01 (s, 4H), 2.72 |
| | | | | (s, 3H), 2.59 (d, J = 15.2 Hz, 3H), |
| | | | | 2.45 (s, 4H), 2.43 (s, 1H), 2.28- |
| | | | | 2.23 (m, 1H), 2.21-2.11 (m, 7H), |
| | | | | 2.04-1.94 (m, 3H), 1.80 (dt, J = |
| | | | | 4.2, 8.4 Hz, 1H), 1.74-1.66 (m, |
| | | | | 2H), 1.56-1.43 (m, 5H), 1.41- |
| | | | | 1.35 (m, 5H), 1.28-1.20 (m, 2H), |
| | | | | 0.93 (d, J = 4.4 Hz, 15H) |
| I-75 | PI | OB | 1494.6 | 11.54 (s, 1H), 8.99-8.96 (m, 1H), |
| | | | | 8.36 (d, J = 8.0 Hz, 1H), 8.14 (s, |
| | | | | 1H), 8.01 (s, 1H), 7.94 (d, J = 2.4 |
| | | | | Hz, 1H), 7.80 (d, J = 9.2 Hz, 2H), |
| | | | | 7.56 (d, J = 8.8 Hz, 1H), 7.45- |
| | | | | 7.41 (m, 3H), 7.39-7.32 (m, 5H), |
| | | | | 7.04 (d, J = 8.4 Hz, 2H), 7.00 (d, |
| | | | | J = 7.6 Hz, 1H), 6.85 (d, J = 8.4 |
| | | | | Hz, 1H), 6.65-6.59 (m, 1H), 6.35- |
| | | | | 6.30 (m, 1H), 6.23 (d, J = 2.0 |
| | | | | Hz, 1H), 5.10 (s, 1H), 4.96-4.87 |
| | | | | (m, 1H), 4.52 (d, J = 9.2 Hz, 1H), |
| | | | | 4.41 (t, J = 8.1 Hz, 1H), 4.28 (s, |
| | | | | 1H), 3.63-3.56 (m, 2H), 3.24 (s, |
| | | | | 3H), 3.00 (s, 4H), 2.83 (d, J = 3.6 |
| | | | | Hz, 2H), 2.72 (s, 3H), 2.45 (s, |
| | | | | 3H), 2.29-2.23 (m, 1H), 2.22- |
| | | | | 2.10 (m, 8H), 2.04-1.98 (m, 1H), |
| | | | | 1.95 (s, 2H), 1.84-1.76 (m, 4H), |
| | | | | 1.60-1.44 (m, 5H), 1.41-1.32 |
| | | | | (m, 7H), 1.30-1.18 (m, 3H), 0.93 |
| | | | | (d, J = 3.2 Hz, 15H) |
| I-77 | PM | OB | 1454.5 | 11.65 (s, 1H), 9.08-8.81 (m, 1H), |
| | | | | 8.37 (d, J = 8.0 Hz, 1H), 8.12 (d, |
| | | | | J = 9.6 Hz, 1H), 8.01 (d, J = 2.0 |
| | | | | Hz, 1H), 7.90 ( d, J = 8.4 Hz, 1H), |
| | | | | 7.79 (d, J = 9.2 Hz, 1H), 7.52- |
| | | | | 7.47 (m, 2H), 7.44-7.41 (m, 2H), |
| | | | | 7.39-7.36 (m, 2H), 7.34 (d, J = |
| | | | | 8.4 Hz, 2H), 7.04 (d, J = 8.4 Hz, |
| | | | | 2H), 6.97-6.86 (m, 1H), 6.65 (d, |
| | | | | J = 7.6 Hz, 1H), 6.37 (s, 1H), 6.20 |
| | | | | (s, 1H), 5.09 (d, J = 2.4 Hz, 1H), |
| | | | | 4.91 (quin, J = 6.8 Hz, 1H), 4.51 |
| | | | | (d, J = 9.2 Hz, 1H), 4.42 (t, J = 8.0 |
| | | | | Hz, 1H), 4.27 (s, 1H), 3.63-3.55 |
| | | | | (m, 2H), 3.04 (s, 4H), 2.73 (s, |
| | | | | 3H), 2.45-2.44 (m, 3H), 2.32 (d, |
| | | | | J = 2.0 Hz, 1H), 2.25 (td, J = 7.6, |
| | | | | 14.4 Hz, 3H), 2.18 (s, 4H), 2.13 |
| | | | | (d, J = 6.8 Hz, 2H), 2.11-2.07 |
| | | | | (m, 1H), 2.03-1.98 (m, 1H), 1.95 |
| | | | | (s, 2H), 1.79 (dt, J = 4.4, 8.4 Hz, |
| | | | | 1H), 1.53-1.44 (m, 4H), 1.40- |
| | | | | 1.35 (m, 5H), 1.29-1.22 (m, 2H), |
| | | | | 0.92 (d, J = 1.2 Hz, 15H) |
| I-83 | LR | OB | 1368.6 | 8.99 (s, 1H), 8.46 (d, J = 2.0 Hz, |
| | | | | 1H), 8.37 (d, J = 7.6 Hz, 1H), 8.26 |
| | | | | (d, J = 8.8 Hz, 1H), 7.81 (d, J = |
| | | | | 9.2 Hz, 2H), 7.73 (d, J = 8.8 Hz, |
| | | | | 2H), 7.46-7.36 (m, 8H), 7.34- |
| | | | | 7.30 (m, 2H), 7.25 (t, J = 7.6 Hz, |
| | | | | 2H), 7.20-7.15 (m, 1H), 6.98 (d, |
| | | | | J = 8.4 Hz, 1H), 6.89 (d, J = 9.2 |
| | | | | Hz, 1H), 6.80 (d, J = 9.2 Hz, 2H), |
| | | | | 6.73 (d, J = 2.0 Hz, 1H), 6.53 (dd, |
| | | | | J = 2.0, 8.4 Hz, 1H), 5.79-5.62 |
| | | | | (m, 1H), 5.11 (s, 1H), 4.98-4.87 |
| | | | | (m, 1H), 4.53 (d, J = 9.4 Hz, 1H), |
| | | | | 4.43 (t, J = 8.0 Hz, 1H), 4.29 (s, |
| | | | | 1H), 4.15-3.98 (m, 1H), 3.61 (s, |
| | | | | 2H), 3.32-3.30 (m, 4H), 3.19- |
| | | | | 3.11 (m, 4H), 3.02 (s, 2H), 2.91- |
| | | | | 2.72 (m, 2H), 2.54-2.52 (m, 2H), |
| | | | | 2.48 (s, 3H), 2.46 (s, 3H), 2.42 (d, |
| | | | | J = 6.8 Hz, 4H), 2.35-2.22 (m, |
| | | | | 2H), 2.15 (dd, J = 6.4, 14.1 Hz, |
| | | | | 1H), 2.07-1.90 (m, 3H), 1.80 |
| | | | | (ddd, J = 4.4, 8.4, 13.0 Hz, 1H), |
| | | | | 1.63-1.44 (m, 5H), 1.41-1.32 |
| | | | | (m, 5H), 0.94 (s, 9H) |
| I-94 | PJ | ML | 1551.6 | 11.65-11.43 (m, 1H), 9.04-8.92 |
| | | | | (m, 1H), 8.39-8.35 (m, 1H), 8.03- |
| | | | | 7.98 (m, 1H), 7.96-7.89 (m, |
| | | | | 1H), 7.87-7.76 (m, 2H), 7.58 (d, |
| | | | | J = 8.8 Hz, 1H), 7.45-7.40 (m, |
| | | | | 3H), 7.38 (s, 1H), 7.37-7.32 (m, |
| | | | | 3H), 7.31 (br s, 1H), 7.08-7.03 |
| | | | | (m, 2H), 7.00 (td, J = 2.4, 5.0 Hz, |
| | | | | 1H), 6.90-6.80 (m, 1H), 6.65- |
| | | | | 6.58 (m, 1H), 6.35-6.30 (m, 1H), |
| | | | | 6.23 (s, 1H), 5.15-5.06 (m, 1H), |
| | | | | 4.97-4.87 (m, 1H), 4.56-4.49 |
| | | | | (m, 1H), 4.47-4.36 (m, 2H), 4.28 |
| | | | | (br s, 1H), 3.98-3.85 (m, 1H), |
| | | | | 3.69-3.52 (m, 2H), 3.46-3.38 |
| | | | | (m, 3H), 3.27-3.15 (m, 2H), 3.03- |
| | | | | 2.96 (m, 5H), 2.95-2.87 (m, |
| | | | | 2H), 2.75-2.69 (m, 2H), 2.54 (s, |
| | | | | 2H), 2.45 (s, 3H), 2.34-2.26 (m, |
| | | | | 3H), 2.17 (br d, J = 14.8 Hz, 7H), |
| | | | | 2.03-1.99 (m, 1H), 1.98-1.90 |
| | | | | (m, 4H), 1.84-1.75 (m, 3H), 1.46 |
| | | | | (d, J = 6.4 Hz, 4H), 1.37 (d, J = |
| | | | | 7.2 Hz, 4H), 1.29-1.18 (m, 2H), |
| | | | | 0.95-0.91 (m, 15H) |
| I-98 | LB | LV | 1551.6 | 11.59 (s, 1H), 8.99 (s, 1H), 8.37 |
| | | | | (d, J = 8.0 Hz, 1H), 8.04 (s, 1H), |
| | | | | 7.97 (d, J = 2.4 Hz, 1H), 7.84- |
| | | | | 7.77 (m, 2H), 7.54 (d, J = 8.8 Hz, |
| | | | | 1H), 7.45 (d, J = 4.0 Hz, 2H), 7.43 |
| | | | | (s, 1H), 7.39 (s, 1H), 7.37 (d, J = |
| | | | | 4.8 Hz, 3H), 7.34 (s, 1H), 7.05 (d, |
| | | | | J = 8.4 Hz, 3H), 6.85 (d, J = 8.0 |
| | | | | Hz, 1H), 6.64 (d, J = 8.8 Hz, 1H), |
| | | | | 6.35 (s, 1H), 6.23 (s, 1H), 5.16- |
| | | | | 5.06 (m, 1H), 4.96-4.89 (m, 1H), |
| | | | | 4.53 (d, J = 9.6 Hz, 1H), 4.42 (t, |
| | | | | J = 8.0 Hz, 1H), 4.29 (s, 1H), 3.67- |
| | | | | 3.54 (m, 3H), 3.02 (s, 6H), 2.73 |
| | | | | (s, 3H), 2.46 (s, 5H), 2.30-2.23 |
| | | | | (m, 2H), 2.23-2.12 (m, 9H), 2.12- |
| | | | | 2.06 (m, 1H), 2.00 (d, J = 9.6 |
| | | | | Hz, 2H), 1.96 (s, 2H), 1.80 (dt, |
| | | | | J = 4.4, 8.3 Hz, 1H), 1.72 (s, 2H), |
| | | | | 1.53-1.44 (m, 5H), 1.41-1.36 |
| | | | | (m, 6H), 1.26 (s, 8H), 0.94 (d, J = |
| | | | | 3.2 Hz, 15H) |
| I-100^{b} | PN | PP | 1498.6 | 11.58 (s, 1H), 9.00-8.97 (m, 1H), |
| | | | | 8.37 (d, J = 8.0 Hz, 1H), 8.05 (s, |
| | | | | 1H), 7.97 (s, 1H), 7.89-7.77 (m, |
| | | | | 2H), 7.56 (d, J = 8.8 Hz, 1H), 7.47- |
| | | | | 7.42 (m, 3H), 7.40-7.33 (m, |
| | | | | 5H), 7.05 (d, J = 8.4 Hz, 3H), 6.95- |
| | | | | 6.81 (m, 1H), 6.64 (d, J = 7.6 |
| | | | | Hz, 1H), 6.35 (s, 1H), 6.23 (s, |
| | | | | 1H), 5.11 (d, J = 3.2 Hz, 1H), 4.98- |
| | | | | 4.88 (m, 1H), 4.56-4.50 (m, |
| | | | | 1H), 4.42 (t, J = 8.0 Hz, 1H), 4.29 |
| | | | | (s, 1H), 3.65-3.55 (m, 4H), 3.39- |
| | | | | 3.36 (m, 2H), 3.02 (s, 4H), 2.96- |
| | | | | 2.81 (m, 4H), 2.73 (s, 2H), 2.53 |
| | | | | (s, 2H), 2.46-2.45 (m, 3H), 2.25 |
| | | | | (s, 1H), 2.22-2.10 (m, 8H), 2.05- |
| | | | | 1.98 (m, 1H), 1.96 (s, 2H), 1.80 |
| | | | | (dt, J = 4.0, 8.4 Hz, 3H), 1.60- |
| | | | | 1.43 (m, 5H), 1.41-1.35 (m, 5H), |
| | | | | 1.28-1.20 (m, 2H), 0.93 (d, J = |
| | | | | 1.6 Hz, 15H) |
| I-108 | OX | MA | 1455.6 | 8.98 (s, 1H), 8.37 (br d, J = 8.0 |
| | | | | Hz, 1H), 8.06 (s, 1H), 7.97-7.91 |
| | | | | (m, 1H), 7.78 (br d, J = 9.6 Hz, |
| | | | | 1H), 7.66 (d, J = 8.8 Hz, 2H), 7.58 |
| | | | | (d, J = 7.6 Hz, 1H), 7.48 (d, J = |
| | | | | 8.4 Hz, 2H), 7.45-7.41 (m, 3H), |
| | | | | 7.40-7.37 (m, 2H), 7.34 (br d, J = |
| | | | | 6.4 Hz, 5H), 7.31-7.26 (m, |
| | | | | 3H), 7.23-7.17 (m, 1H), 7.15- |
| | | | | 7.10 (m, 1H), 6.84 (br dd, J = 2.4, |
| | | | | 5.6 Hz, 1H), 6.71 (br d, J = 8.8 |
| | | | | Hz, 2H), 5.25-5.19 (m, 1H), 5.11 |
| | | | | (d, J = 3.2 Hz, 1H), 4.94-4.88 |
| | | | | (m, 1H), 4.51 (br d, J = 9.2 Hz, |
| | | | | 1H), 4.41 (t, J = 8.0 Hz, 1H), 4.33- |
| | | | | 4.24 (m, 2H), 4.03-3.92 (m, |
| | | | | 1H), 3.75-3.66 (m, 1H), 3.63- |
| | | | | 3.51 (m, 3H), 2.95-2.90 (m, 1H), |
| | | | | 2.55 (br s, 1H), 2.45 (s, 4H), 2.28- |
| | | | | 2.18 (m, 2H), 2.07 (s, 10H), 2.02- |
| | | | | 1.99 (m, 1H), 1.89 (br dd, J = |
| | | | | 3.2, 8.8 Hz, 2H), 1.82-1.75 (m, |
| | | | | 1H), 1.64-1.53 (m, 2H), 1.53- |
| | | | | 1.41 (m, 4H), 1.37 (br d, J = 6.4 |
| | | | | Hz, 4H), 1.22 (br d, J = 7.6 Hz, |
| | | | | 4H), 1.18-1.09 (m, 2H), 0.92 (s, |
| | | | | 9H) |
| I-118 | KS | MA | 1510.5 | 12.10-11.91 (m, 1H), 11.76- |
| | | | | 11.37 (m, 1H), 9.00 (s, 1H), 8.40 |
| | | | | (d, J = 7.8 Hz, 1H), 8.17 (d, J = |
| | | | | 2.0 Hz, 1H), 7.98 (dd, J = 2.0, 9.2 |
| | | | | Hz, 1H), 7.81 (d, J = 9.2 Hz, 1H), |
| | | | | 7.68 (d, J = 8.8 Hz, 2H), 7.58 (d, |
| | | | | J = 8.0 Hz, 1H), 7.47 (d, J = 8.4 |
| | | | | Hz, 2H), 7.45-7.40 (m, 3H), 7.40- |
| | | | | 7.36 (m, 2H), 7.35-7.27 (m, |
| | | | | 5H), 7.25-7.19 (m, 3H), 7.14 (t, |
| | | | | J = 8.0 Hz, 2H), 6.99 (d, J = 8.4 |
| | | | | Hz, 1H), 6.84 (d, J = 9.2 Hz, 2H), |
| | | | | 4.95-4.86 (m, 1H), 4.56-4.48 |
| | | | | (m, 1H), 4.42 (t, J = 8.0 Hz, 1H), |
| | | | | 4.32-4.25 (m, 2H), 4.23-4.16 |
| | | | | (m, 1H), 3.92-3.82 (m, 2H), 3.71 |
| | | | | (d, J = 1.2 Hz, 2H), 3.59 (s, 3H), |
| | | | | 3.39-3.35 (m, 4H), 3.12-3.01 |
| | | | | (m, 3H), 2.74-2.68 (m, 1H), 2.61- |
| | | | | 2.58 (m, 2H), 2.54 (s, 1H), 2.45 |
| | | | | (s, 3H), 2.29-2.19 (m, 2H), 2.14 |
| | | | | (dt, J = 6.8, 13.7 Hz, 3H), 2.05- |
| | | | | 1.99 (m, 1H), 1.91-1.85 (m, 1H), |
| | | | | 1.78 (td, J = 4.0, 8.8 Hz, 1H), 1.67 |
| | | | | (d, J = 7.2 Hz, 3H), 1.57-1.42 |
| | | | | (m, 3H), 1.37 (d, J = 7.2 Hz, 3H), |
| | | | | 1.32-1.21 (m, 5H), 1.12-1.06 |
| | | | | (m, 1H), 1.04-0.97 (m, 2H), 0.93 |
| | | | | (s, 9H), 0.85-0.78 (m, 1H) |
| I-122 | PS | PP | 1398.5 | 11.55 (br s, 1H), 9.03-8.91 (m, |
| | | | | 1H), 8.41 (br d, J = 7.6 Hz, 2H), |
| | | | | 8.02 (br s, 1H), 7.95 (d, J = 2.4 |
| | | | | Hz, 1H), 7.82 (br d, J = 8.0 Hz, |
| | | | | 1H), 7.57 (br d, J = 8.8 Hz, 1H), |
| | | | | 7.44 (br d, J = 8.0 Hz, 3H), 7.40- |
| | | | | 7.31 (m, 5H), 7.05 (br d, J = 8.4 |
| | | | | Hz, 3H), 6.82 (br d, J = 8.0 Hz, |
| | | | | 1H), 6.66-6.59 (m, 1H), 6.33 (br |
| | | | | s, 1H), 6.24 (s, 1H), 5.13 (d, J = |
| | | | | 3.2 Hz, 1H), 4.92 (quin, J = 7.2 |
| | | | | Hz, 1H), 4.55 (br d, J = 9.2 Hz, |
| | | | | 1H), 4.43 (br t, J = 8.0 Hz, 1H), |
| | | | | 4.30 (br s, 1H), 3.70-3.53 (m, |
| | | | | 3H), 3.39 (br s, 2H), 3.00 (br s, |
| | | | | 4H), 2.85-2.70 (m, 4H), 2.47- |
| | | | | 2.43 (m, 3H), 2.23-2.13 (m, 7H), |
| | | | | 2.07-2.00 (m, 1H), 1.96 (br s, |
| | | | | 2H), 1.84-1.74 (m, 3H), 1.42- |
| | | | | 1.34 (m, 5H), 1.24 (s, 3H), 0.94 |
| | | | | (br d, J = 7.6 Hz, 15H) |
| I-137 | LB | MA | 1537.6 | 11.58 (br s, 1H), 8.98 (s, 1H), |
| | | | | 8.37 (d, J = 8.0 Hz, 1H), 8.03 (s, |
| | | | | 1H), 7.96 (d, J = 2.4 Hz, 1H), 7.84- |
| | | | | 7.77 (m, 2H), 7.54 (d, J = 8.8 |
| | | | | Hz, 1H), 7.46-7.33 (m, 9H), 7.05 |
| | | | | (d, J = 8.4 Hz, 3H), 6.90-6.79 |
| | | | | (m, 1H), 6.67-6.61 (m, 1H), 6.35 |
| | | | | (br s, 1H), 6.23 (d, J = 1.6 Hz, |
| | | | | 1H), 5.11 (d, J = 3.6 Hz, 1H), 4.92 |
| | | | | (t, J = 6.8 Hz, 1H), 4.52 (d, J = 9.2 |
| | | | | Hz, 1H), 4.42 (t, J = 8.0 Hz, 1H), |
| | | | | 4.29 (br s, 1H), 3.64-3.56 (m, |
| | | | | 2H), 3.46-3.42 (m, 6H), 3.02 (br |
| | | | | s, 4H), 2.73 (br s, 3H), 2.58-2.53 |
| | | | | (m, 4H), 2.48-2.44 (m, 4H), 2.43- |
| | | | | 2.34 (m, 2H), 2.19 (br s, 1H), |
| | | | | 2.31-2.06 (m, 6H), 2.06-1.98 |
| | | | | (m, 1H), 1.96 (br s, 2H), 1.80 |
| | | | | (ddd, J = 4.7, 8.4, 12.8 Hz, 1H), |
| | | | | 1.76-1.66 (m, 2H), 1.56-1.45 |
| | | | | (m, 4H), 1.41-1.36 (m, 5H), 1.26 |
| | | | | (br s, 4H), 0.94 (br s, 9H), 0.93- |
| | | | | 0.91 (m, 6H) |
| 1-154 | PH | OB | 1488.7 | 11.58-11.50 (m, 1H), 8.99 (s, |
| | | | | 1H), 8.37 (d, J = 7.6 Hz, 1H), 8.08- |
| | | | | 7.87 (m, 2H), 7.84-7.72 (m, |
| | | | | 2H), 7.59-7.48 (m, 2H), 7.46- |
| | | | | 7.41 (m, 3H), 7.40-7.36 (m, 2H), |
| | | | | 7.34-7.27 (m, 1H), 7.24-7.19 |
| | | | | (m, 1H), 7.12 (d, J = 5.6 Hz, 2H), |
| | | | | 7.03-6.90 (m, 1H), 6.89-6.76 |
| | | | | (m, 1H), 6.61 (d, J = 9.2 Hz, 1H), |
| | | | | 6.32 (s, 1H), 6.21 (s, 1H), 5.11 (d, |
| | | | | J = 3.2 Hz, 1H), 4.98-4.88 (m, |
| | | | | 1H), 4.53 (d, J = 9.6 Hz, 1H), 4.42 |
| | | | | (t, J = 8.0 Hz, 1H), 4.29 (s, 1H), |
| | | | | 3.68-3.55 (m, 3H), 3.36 (s, 1H), |
| | | | | 3.28-3.19 (m, 4H), 3.13-3.03 |
| | | | | (m, 2H), 2.99-2.84 (m, 6H), 2.55 |
| | | | | (s, 1H), 2.46 (s, 3H), 2.26 (dd, J = |
| | | | | 7.2, 14.4 Hz, 2H), 2.14 (dd, J = |
| | | | | 6.4, 13.9 Hz, 2H), 2.05-1.98 (m, |
| | | | | 1H), 1.84-1.66 (m, 8H), 1.61- |
| | | | | 1.42 (m, 9H), 1.38 (d, J = 6.8 Hz, |
| | | | | 8H), 1.29-1.22 (m, 3H), 1.16 (t, |
| | | | | J = 6.0 Hz, 6H), 0.94 (s, 9H) |
| I-163 | QW | QZ | 1493.5 | 11.59 (d, J = 2.0 Hz, 1H), 9.00- |
| | | | | 8.96 (m, 1H), 8.36 (d, J = 8.0 Hz, |
| | | | | 1H), 8.04 (d, J = 0.8 Hz, 1H), 7.95 |
| | | | | (d, J = 1.2 Hz, 1H), 7.87-7.77 |
| | | | | (m, 2H), 7.52 (t, J = 8.4 Hz, 2H), |
| | | | | 7.49-7.41 (m, 5H), 7.40-7.35 |
| | | | | (m, 3H), 7.35-7.25 (m, 4H), 7.21- |
| | | | | 7.04 (m, 2H), 6.95-6.77 (m, |
| | | | | 1H), 6.63-6.53 (m, 1H), 6.34 (s, |
| | | | | 1H), 6.15 (s, 1H), 5.19 (d, J = 4.4 |
| | | | | Hz, 1H), 5.10 (d, J = 3.2 Hz, 1H), |
| | | | | 4.96-4.86 (m, 1H), 4.51 (d, J = |
| | | | | 9.2 Hz, 1H), 4.41 (t, J = 8.4 Hz, |
| | | | | 1H), 4.32-4.21 (m, 2H), 3.65- |
| | | | | 3.52 (m, 3H), 3.42-3.36 (m, 4H), |
| | | | | 3.05-2.86 (m, 2H), 2.79-2.69 |
| | | | | (m, 1H), 2.53-2.51 (m, 2H), 2.45 |
| | | | | (s, 3H), 2.43-2.34 (m, 2H), 2.31- |
| | | | | 2.25 (m, 1H), 2.25-2.12 (m, |
| | | | | 2H), 2.04-1.98 (m, 1H), 1.90- |
| | | | | 1.74 (m, 6H), 1.69 (dd, J = 6.0, |
| | | | | 12.4 Hz, 2H), 1.62-1.48 (m, 3H), |
| | | | | 1.37 (d, J = 7.2 Hz, 3H), 1.32- |
| | | | | 1.21 (m, 1H), 1.09-1.01 (m, 1H), |
| | | | | 0.93 (s, 10H), 0.89-0.83 (m, 1H), |
| | | | | 0.81-0.72 (m, 1H) |
| I-164 | QW | RA | 1493.5 | 11.69-11.50 (m, 1H), 9.01-8.95 |
| | | | | (m, 1H), 8.36 (d, J = 8.0 Hz, 1H), |
| | | | | 8.09-8.01 (m, 1H), 7.99-7.91 |
| | | | | (m, 1H), 7.88-7.77 (m, 2H), 7.55- |
| | | | | 7.49 (m, 2H), 7.48-7.41 (m, |
| | | | | 5H), 7.40-7.35 (m, 3H), 7.35- |
| | | | | 7.29 (m, 3H), 7.29-7.26 (m, 1H), |
| | | | | 7.19-7.05 (m, 2H), 6.97-6.81 |
| | | | | (m, 1H), 6.62-6.54 (m, 1H), 6.37- |
| | | | | 6.30 (m, 1H), 6.20-6.10 (m, |
| | | | | 1H), 5.19 (d, J = 4.4 Hz, 1H), 5.10 |
| | | | | (d, J = 3.6 Hz, 1H), 4.96-4.87 |
| | | | | (m, 1H), 4.51 (d, J = 9.2 Hz, 1H), |
| | | | | 4.45-4.37 (m, 1H), 4.31-4.21 |
| | | | | (m, 2H), 3.65-3.51 (m, 3H), 3.41- |
| | | | | 3.35 (m, 4H), 3.06-2.75 (m, |
| | | | | 3H), 2.53-2.51 (m, 2H), 2.45 (s, |
| | | | | 3H), 2.43-2.34 (m, 2H), 2.31- |
| | | | | 2.25 (m, 1H), 2.25-2.11 (m, 2H), |
| | | | | 2.04-1.98 (m, 1H), 1.91-1.74 |
| | | | | (m, 6H), 1.74-1.64 (m, 3H), 1.64- |
| | | | | 1.42 (m, 3H), 1.37 (d, J = 7.2 |
| | | | | Hz, 3H), 1.08-0.99 (m, 1H), 0.93 |
| | | | | (s, 11H), 0.81-0.72 (m, 1H) |
| 1-167 | NV | LV | 1636.7 | 11.58 (br s, 1H), 9.00-8.96 (m, |
| | | | | 1H), 8.36 (d, J = 7.6 Hz, 1H), 8.02 |
| | | | | (s, 1H), 7.96 (d, J = 2.4 Hz, 1H), |
| | | | | 7.78 (br d, J = 9.2 Hz, 2H), 7.55 |
| | | | | (d, J = 8.8 Hz, 1H), 7.45 (s, 2H), |
| | | | | 7.41 (d, J = 14.4 Hz, 3H), 7.38- |
| | | | | 7.32 (m, 4H), 7.09 (br d, J = 8.4 |
| | | | | Hz, 3H), 6.81 (br d, J = 9.2 Hz, |
| | | | | 1H), 6.64 (br d, J = 9.2 Hz, 1H), |
| | | | | 6.34 (dd, J = 2.0, 3.2 Hz, 1H), |
| | | | | 6.24 (s, 1H), 5.11 (br d, J = 3.2 |
| | | | | Hz, 1H), 4.92 (t, J = 7.2 Hz, 1H), |
| | | | | 4.53 (d, J = 9.2 Hz, 1H), 4.42 (t, |
| | | | | J = 8.0 Hz, 1H), 4.29 (br s, 1H), |
| | | | | 3.64-3.56 (m, 7H), 3.01 (br s, |
| | | | | 4H), 2.85-2.78 (m, 2H), 2.69 (br |
| | | | | s, 4H), 2.62 (br d, J = 4.8 Hz, 3H), |
| | | | | 2.46 (s, 3H), 2.36 (br d, J = 6.0 |
| | | | | Hz, 6H), 2.30-2.24 (m, 2H), 2.20 |
| | | | | (br s, 8H), 2.12 (br d, J = 7.2 Hz, |
| | | | | 2H), 2.03 (br s, 2H), 1.88 (br s, |
| | | | | 1H), 1.85-1.73 (m, 2H), 1.70 (br |
| | | | | t, J = 6.4 Hz, 2H), 1.56-1.44 (m, |
| | | | | 6H), 1.38 (d, .J = 7.2 Hz, 4H), 1.26 |
| | | | | (br s, 6H), 0.94 (s, 9H), 0.89 (s, |
| | | | | 3H) |
| I-175 | PK | MA | 1482.5 | 11.90 (s, 1H), 11.76 (s, 1H), |
| | | | | 10.84 (s, 1H), 10.68-10.58 (m, |
| | | | | 1H), 9.06 (s, 1H), 8.43 (d, J = 7.8 |
| | | | | Hz, 1H), 8.18 (d, J = 2.0 Hz, 1H), |
| | | | | 8.09 (d, J = 2.4 Hz, 1H), 7.96 (dd, |
| | | | | J = 2.0, 9.2 Hz, 1H), 7.81 (d, J = |
| | | | | 9.2 Hz, 1H), 7.65 (d, J = 2.4 Hz, |
| | | | | 1H), 7.56 (t, J = 2.8 Hz, 1H), 7.52- |
| | | | | 7.46 (m, 2H), 7.45-7.42 (m, |
| | | | | 2H), 7.38 (d, J = 8.0 Hz, 4H), 7.16 |
| | | | | (d, J = 9.6 Hz, 1H), 7.09 (d, J = |
| | | | | 8.4 Hz, 2H), 6.74-6.70 (m, 1H), |
| | | | | 6.46 (dd, J = 1.6, 3.2 Hz, 1H), |
| | | | | 6.25 (d, J = 1.6 Hz, 1H), 4.93- |
| | | | | 4.89 (m, 1H), 4.51 (d, J = 9.2 Hz, |
| | | | | 1H), 4.42 (t, J = 8.0 Hz, 1H), 4.27 |
| | | | | (s, 1H), 3.63 (s, 1H), 3.60 (s, 2H), |
| | | | | 3.52 (d, J = 3.2 Hz, 2H), 3.46 (d, |
| | | | | J = 6.4 Hz, 2H), 3.33-3.24 (m, |
| | | | | 4H), 3.13-3.07 (m, 1H), 3.06- |
| | | | | 2.89 (m, 4H), 2.69 (d, J = 4.8 Hz, |
| | | | | 4H), 2.66 (s, 1H), 2.46 (s, 3H), |
| | | | | 2.36 (s, 2H), 2.27-2.21 (m, 1H), |
| | | | | 2.17-2.11 (m, 1H), 2.05 (s, 1H), |
| | | | | 2.00 (s, 2H), 1.98-1.92 (m, 2H), |
| | | | | 1.78 (s, 1H), 1.64 (s, 2H), 1.50- |
| | | | | 1.45 (m, 2H), 1.43 (t, J = 6.0 Hz, |
| | | | | 2H), 1.37 (d, J = 7.2 Hz, 3H), 1.26 |
| | | | | (s, 5H), 0.93 (s, 15H) |
| I-177^{c} | PK | RR | 1439.6 | 11.61-11.50 (m, 1H), 8.35 (br d, |
| | | | | J = 8.4 Hz, 1H), 8.05-7.90 (m, |
| | | | | 2H), 7.81 (br d, J = 9.2 Hz, 2H), |
| | | | | 7.57 (br d, J = 8.4 Hz, 1H), 7.46- |
| | | | | 7.39 (m, 3H), 7.33 (br dd, J = 8.4, |
| | | | | 16.9 Hz, 4H), 7.05 (br d, J = 8.4 |
| | | | | Hz, 2H), 6.88-6.76 (m, 1H), 6.63 |
| | | | | (br d, J = 10.0 Hz, 1H), 6.33 (br s, |
| | | | | 1H), 6.24 (s, 1H), 5.11 (d, J = 3.6 |
| | | | | Hz, 1H), 4.83 (br d, J = 7.6 Hz, |
| | | | | 1H), 4.75 (s, 1H), 4.52 (br d, J = |
| | | | | 8.8 Hz, 1H), 4.44 (s, 1H), 4.29- |
| | | | | 4.23 (m, 1H), 4.14 (s, 1H), 3.61 |
| | | | | (br s, 2H), 3.55 (br s, 3H), 3.01 (br |
| | | | | s, 4H), 2.73 (br s, 4H), 2.55 (br s, |
| | | | | 3H), 2.24 (br s, 3H), 2.22-2.14 |
| | | | | (m, 7H), 2.13-2.07 (m, 2H), 1.96 |
| | | | | (br s, 3H), 1.84-1.75 (m, 3H), |
| | | | | 1.56-1.43 (m, 5H), 1.39 (br s, |
| | | | | 2H), 1.25 (br d, J = 1.2 Hz, 6H), |
| | | | | 0.93 (s, 15H) |
| I-190^{c} | NV | RR | 1579.6 | 11.65-11.45 (m, 1H), 8.38-8.27 |
| | | | | (m, 1H), 8.07-7.90 (m, 2H), 7.84- |
| | | | | 7.72 (m, 2H), 7.54 (d, J = 8.8 |
| | | | | Hz, 1H), 7.47-7.26 (m, 9H), 7.08 |
| | | | | (d, J = 8.4 Hz, 3H), 6.87-6.74 |
| | | | | (m, 1H), 6.63 (dd, J = 1.6, 9.1 Hz, |
| | | | | 1H), 6.37-6.28 (m, 1H), 6.26- |
| | | | | 6.17 (m, 1H), 5.14-5.07 (m, 1H), |
| | | | | 4.86-4.79 (m, 1H), 4.74 (t, J = |
| | | | | 6.0 Hz, 1H), 4.51 (d, J = 9.2 Hz, |
| | | | | 1H), 4.47-4.39 (m, 1H), 4.31- |
| | | | | 4.22 (m, 1H), 4.13 (s, 1H), 3.62- |
| | | | | 3.54 (m, 8H), 3.00 (s, 4H), 2.67 |
| | | | | (td, J = 1.8, 3.6 Hz, 4H), 2.55- |
| | | | | 2.52 (m, 4H), 2.33 (dd, J = 2.4, |
| | | | | 4.4 Hz, 6H), 2.29-2.23 (m, 2H), |
| | | | | 2.19 (s, 8H), 2.13-2.05 (m, 2H), |
| | | | | 2.03-1.95 (m, 2H), 1.87 (s, 1H), |
| | | | | 1.84-1.72 (m, 2H), 1.69 (t, J = |
| | | | | 6.4 Hz, 2H), 1.54-1.33 (m, 7H), |
| | | | | 1.24 (d, J = 6.0 Hz, 8H), 0.93 (s, |
| | | | | 9H), 0.88 (s, 3H) |
| I-199^{d} | C | AT | 1587.1 | 9.01-8.96 (m, 1H), 8.48-8.41 |
| | | | | (m, 1H), 8.08-8.05 (m, 1H), 7.96- |
| | | | | 7.89 (m, 1H), 7.70 (br d, J = 8.4 |
| | | | | Hz, 2H), 7.42-7.32 (m, 6H), 7.29 |
| | | | | (br t, J = 7.6 Hz, 3H), 7.23-7.16 |
| | | | | (m, 1H), 7.14-7.05 (m, 3H), 6.97- |
| | | | | 6.91 (m, 1H), 6.91-6.85 (m, |
| | | | | 1H), 6.80-6.75 (m, 2H), 6.70 (br |
| | | | | dd, J = 0.8, 6.4 Hz, 1H), 5.17 (d, |
| | | | | J = 3.2 Hz, 1H), 4.62-4.56 (m, |
| | | | | 1H), 4.53-4.48 (m, 1H), 4.40- |
| | | | | 4.32 (m, 2H), 4.30-4.14 (m, 2H), |
| | | | | 4.07-3.98 (m, 1H), 3.67-3.55 |
| | | | | (m, 4H), 3.15-3.07 (m, 4H), 2.78- |
| | | | | 2.63 (m, 8H), 2.45 (br s, 3H), |
| | | | | 2.29-2.10 (m, 11H), 2.02-1.86 |
| | | | | (m, 7H), 1.51-1.35 (m, 8H), 1.28- |
| | | | | 1.17 (m, 5H), 0.96 (br s, 15H) |
| I-200^{d} | C | AT | 1586.3 | 8.98 (s, 1H), 8.50 (s, 1H), 8.05 (s, |
| | | | | 1H), 7.99-7.90 (m, 1H), 7.72- |
| | | | | 7.65 (m, 2H), 7.50 (br d, J = 1.2 |
| | | | | Hz, 1H), 7.40-7.25 (m, 8H), 7.22- |
| | | | | 7.16 (m, 1H), 7.13-7.08 (m, |
| | | | | 2H), 7.03-6.97 (m, 1H), 6.97- |
| | | | | 6.85 (m, 2H), 6.81-6.63 (m, 3H), |
| | | | | 5.18 (d, J = 3.6 Hz, 1H), 4.73 (dt, |
| | | | | J = 2.4, 8.8 Hz, 1H), 4.63-4.50 |
| | | | | (m, 2H), 4.35 (br s, 2H), 4.31- |
| | | | | 4.23 (m, 1H), 4.10-3.97 (m, 2H), |
| | | | | 3.67-3.56 (m, 4H), 3.18-3.03 |
| | | | | (m, 5H), 2.78-2.69 (m, 3H), 2.68- |
| | | | | 2.66 (m, 2H), 2.45 (s, 3H), 2.36- |
| | | | | 2.30 (m, 2H), 2.29-2.17 (m, |
| | | | | 7H), 2.13-1.87 (m, 8H), 1.81- |
| | | | | 1.54 (m, 6H), 1.48-1.37 (m, 4H), |
| | | | | 1.29-1.19 (m, 5H), 0.95 (s, 15H) |
| I-229 | C | LM | 1530.5 | 12.27-12.08 (m, 1H), 9.01 (s, |
| | | | | 1H), 8.59 (br s, 1H), 8.17 (s, 1H), |
| | | | | 7.99 (br d, J = 8.4 Hz, 1H), 7.77 |
| | | | | (br d, J = 8.4 Hz, 2H), 7.46-7.39 |
| | | | | (m, 3H), 7.32-7.26 (m, 3H), 7.22 |
| | | | | (br t, J = 7.2 Hz, 3H), 7.18-7.12 |
| | | | | (m, 3H), 7.06-6.93 (m, 5H), 4.59 |
| | | | | (br d, J = 9.2 Hz, 1H), 4.52 (br t, |
| | | | | J = 8.0 Hz, 1H), 4.49-4.40 (m, |
| | | | | 2H), 4.33 (br d, J = 12.4 Hz, 3H), |
| | | | | 4.28-4.15 (m, 2H), 3.93-3.85 |
| | | | | (m, 2H), 3.73-3.60 (m, 6H), 3.37 |
| | | | | (br d, J = 8.8 Hz, 10H), 2.84- |
| | | | | 2.71 (m, 2H), 2.67 (br s, 1H), 2.46 |
| | | | | (br s, 4H), 2.35-2.25 (m, 6H), |
| | | | | 2.17-2.04 (m, 5H), 1.94-1.85 |
| | | | | (m, 1H), 1.50-1.44 (m, 2H), 1.40- |
| | | | | 1.30 (m, 2H), 1.21 (br d, J = 8.0 |
| | | | | Hz, 2H), 1.00 (s, 6H), 0.93 (s, 9H) |
| I-237^{e, f} | TK | TL | 1546.5 | 8.99 (s, 1H), 8.59-8.50 (m, 1H), |
| | | | | 8.07 (s, 1H), 7.92 (d, J = 8.4 Hz, |
| | | | | 1H), 7.71 (d, J = 8.8 Hz, 2H), 7.44 |
| | | | | (d, J = 7.6 Hz, 1H), 7.37 (d, J = |
| | | | | 8.4 Hz, 2H), 7.34-7.31 (m, 2H), |
| | | | | 7.30-7.23 (m, 3H), 7.18-7.10 |
| | | | | (m, 3H), 7.03 (s, 1H), 6.97 (d, J = |
| | | | | 8.0 Hz, 1H), 6.89 (d, J = 7.6 Hz, |
| | | | | 1H), 6.82-6.71 (m, 3H), 5.18 (d, |
| | | | | J = 3.5 Hz, 1H), 4.59 (d, J = 9.2 |
| | | | | Hz, 1H), 4.53 (t, J = 8.0 Hz, 1H), |
| | | | | 4.38-4.29 (m, 2H), 4.26-3.96 |
| | | | | (m, 4H), 3.68-3.57 (m, 2H), 3.14 |
| | | | | (s, 5H), 2.75 (d, J = 13.2 Hz, 4H), |
| | | | | 2.45 (s, 3H), 2.32-2.16 (m, 7H), |
| | | | | 2.14-2.05 (m, 2H), 2.04-1.85 |
| | | | | (m, 5H), 1.85-1.70 (m, 1H), 1.47- |
| | | | | 1.31 (m, 5H), 1.28-1.09 (m, |
| | | | | 7H), 0.95 (d, J = 5.6 Hz, 17H), |
| | | | | 0.90-0.73 (m, 1H) |
| I-240 | C | UA | 1499.2 | 8.99 (s, 1H), 8.44-8.26 (m, 1H), |
| | | | | 8.08 (s, 1H), 7.98-7.87 (m, 1H), |
| | | | | 7.70 (d, J = 8.8 Hz, 2H), 7.38- |
| | | | | 7.32 (m, 4H), 7.29-7.25 (m, 2H), |
| | | | | 7.20-7.15 (m, 1H), 7.12 (d, J = |
| | | | | 8.4 Hz, 2H), 7.05-6.97 (m, 2H), |
| | | | | 6.93-6.87 (m, 1H), 6.81-6.69 |
| | | | | (m, 3H), 6.24-6.17 (m, 1H), 5.17- |
| | | | | 5.07 (m, 1H), 4.52-4.14 (m, |
| | | | | 7H), 4.07-3.97 (m, 1H), 3.88- |
| | | | | 3.67 (m, 2H), 3.61-3.50 (m, 1H), |
| | | | | 3.46-3.41 (m, 1H), 3.13 (br d, |
| | | | | J = 1.2 Hz, 4H), 2.78-2.69 (m, |
| | | | | 3H), 2.52 (br s, 6H), 2.47-2.44 |
| | | | | (m, 6H), 2.29-2.18 (m, 10H), |
| | | | | 2.15-2.10 (m, 2H), 2.06-1.87 |
| | | | | (m, 6H), 1.43 (br t, J = 6.4 Hz, |
| | | | | 2H), 0.96 (s, 8H), 0.91 (br d, J = |
| | | | | 6.4 Hz, 2H), 0.77 (br dd, J = 6.4, |
| | | | | 16.8 Hz, 3H) |
| I-241 | UC | LM | 1496.3 | 12.08-11.92 (m, 1H), 9.92-9.74 |
| | | | | (m, 1H), 9.00 (s, 1H), 8.54 (br t, |
| | | | | J = 5.8 Hz, 1H), 8.20-8.12 (m, |
| | | | | 1H), 8.04-7.96 (m, 1H), 7.72- |
| | | | | 7.58 (m, 3H), 7.44 (d, J = 7.8 Hz, |
| | | | | 1H), 7.34-7.18 (m, 7H), 7.16- |
| | | | | 7.08 (m, 3H), 7.08-6.94 (m, 3H), |
| | | | | 6.32 (d, J = 8.8 Hz, 1H), 5.08- |
| | | | | 4.98 (m, 1H), 4.58 (br d, J = 9.3 |
| | | | | Hz, 1H), 4.52 (br t, J = 8.3 Hz, |
| | | | | 1H), 4.38-4.26 (m, 5H), 4.18- |
| | | | | 4.10 (m, 1H), 3.94-3.84 (m, 3H), |
| | | | | 3.80 (br d, J = 8.5 Hz, 1H), 3.76- |
| | | | | 3.66 (m, 2H), 3.66-3.58 (m, 2H), |
| | | | | 3.48-3.28 (m, 5H), 3.28-3.20 |
| | | | | (m, 2H), 2.74-2.70 (m, 2H), 2.68- |
| | | | | 2.58 (m, 7H), 2.46 (s, 3H), 2.28- |
| | | | | 1.98 (m, 7H), 1.96-1.78 (m, |
| | | | | 3H), 1.44-1.36 (m, 1H), 1.36- |
| | | | | 1.35 (m, 1H), 1.26-1.18 (m, 2H), |
| | | | | 1.08-0.96 (m, 3H), 0.96-0.88 |
| | | | | (m, 9H), 0.72-0.66 (m, 1H) |
| I-250 | QI | MA | 1551.2 | 11.54 (s, 1H), 8.99 (s, 1H), 8.39- |
| | | | | 8.34 (m, 1H), 7.99 (s, 1H), 7.94 |
| | | | | (d, J = 2.4 Hz, 1H), 7.82-7.74 |
| | | | | (m, 2H), 7.57 (d, J = 8.4 Hz, 1H), |
| | | | | 7.46-7.42 (m, 3H), 7.39 (s, 1H), |
| | | | | 7.38-7.35 (m, 2H), 7.34 (s, 1H), |
| | | | | 7.32-7.29 (m, 1H), 7.08-7.01 |
| | | | | (m, 3H), 6.81-6.74 (m, 1H), 6.62 |
| | | | | (dd, J = 1.6, 8.8 Hz, 1H), 6.34- |
| | | | | 6.30 (m, 1H), 6.25 (d, J = 2.0 Hz, |
| | | | | 1H), 5.14-5.05 (m, 1H), 4.98- |
| | | | | 4.87 (m, 1H), 4.55-4.50 (m, 1H), |
| | | | | 4.42 (t, J = 8.0 Hz, 1H), 4.29 (d, |
| | | | | J = 2.4 Hz, 1H), 3.65-3.56 (m, |
| | | | | 2H), 3.09-3.05 (m, 2H), 3.01 (s, |
| | | | | 5H), 2.87-2.79 (m, 4H), 2.73 (s, |
| | | | | 4H), 2.60 (t, J = 6.8 Hz, 2H), 2.46 |
| | | | | (s, 3H), 2.24 (s, 1H), 2.20 (s, 4H), |
| | | | | 2.16-2.10 (m, 3H), 2.00 (d, J = |
| | | | | 8.0 Hz, 2H), 1.96 (s, 2H), 1.88- |
| | | | | 1.82 (m, 2H), 1.82-1.77 (m, 1H), |
| | | | | 1.74-1.68 (m, 2H), 1.55-1.47 |
| | | | | (m, 4H), 1.42-1.34 (m, 6H), 1.26 |
| | | | | (s, 6H), 0.94 (d, J = 2.0 Hz, 15H) |

The reductive amination was performed under standard conditions. DCM or THF also can be used as a solvent or co-solvent, AcOH, KOAc, or 4 Å molecular sieves employed to promote the reaction when necessary. The reaction was run 1-12 hr at 25° C. The final products were purified under standard techniques. ^{b}The product of the coupling was further deprotected with HCl/dioxane at rt for 2 hrs. The final compound was purified by reverse phase HPLC. ^{c}The product of the coupling was further deprotected with CsF in DMSO at 40-50° C. for 2 hr. The final compound was purified by reverse phase HPLC. ^{d}The diastereomers were separated by prep-HPLC. The absolute stereochemistry of the diastereomers was arbitrarily assigned. ^{e}A ketone was used instead of an aldehyde. ^{f}NaBH₃CN used for the reductive amination with TEA, HOAc in THF/DMF and the reaction was stirred for 16 hrs at 50° C.

### Example 4: Synthesis of Compounds of Formula I (Method 3)

### Synthesis of (2S,4R)-1-((S)-2-(7-(4-(3-((4-(N-(2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)propyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (I-22)

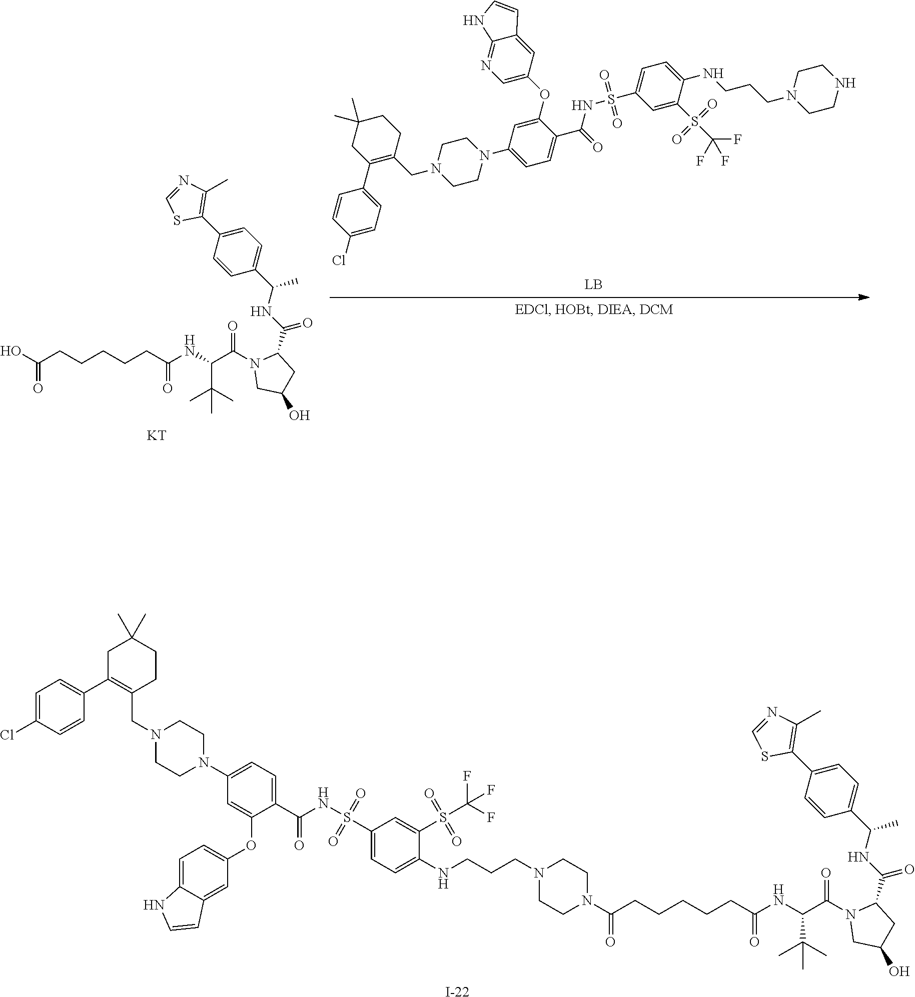

To a solution of 4-[4-[[2-(4-chlorophenyl)-4,4-dimethyl-cyclohexen-1-yl]methyl]piperazin-1-yl]-N-[4-(3-piperazin-1-ylpropylamino)-3-(trifluoromethylsulfonyl)phenyl]sulfonyl-2-(1H-pyrrolo[2,3-b]pyridin-5-yloxy)benzamide (93.4 mg, 92.0 μmol, HCl salt, Intermediate LB) in DCM (1 mL) was added HOBt (34.5 mg, 256 μmol), DIEA (172 mg, 1.33 mmol), EDCI (49.0 mg, 256 μmol) and 7-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-7-oxo-heptanoic acid (60 mg, 102 μmol, Intermediate KT). Then the mixture was stirred at 25° C. for 2 hrs. On completion, the reaction mixture was concentrated under reduced pressure to give a residue. The residue was purified by prep-HPLC (FA condition). Then, 1M HCl (1 mL) was added to the solution and the residue was dried by lyophilization to give the title compound (36 mg, 21% yield, HCl salt) as a white solid. ¹H NMR (400 M Hz, DMSO-d₆) δ ppm 11.74 (br d, J=17.2 Hz, 2H), 10.68-10.78 (m, 1H), 10.14 (td, J=10.4, 4.0 Hz, 1H), 9.00 (s, 1H), 8.38 (d, J=8.0 Hz, 1H), 8.20 (d, J=2.4 Hz, 1H), 8.07 (d, J=2.4 Hz, 1H), 7.97 (dd, J=9.2, 2.0 Hz, 1H), 7.80 (br d, J=8.8 Hz, 1H), 7.60 (d, J=2.4 Hz, 1H), 7.54-7.57 (m, 1H), 7.52 (s, 1H), 7.50 (s, 1H), 7.45 (s, 1H), 7.43 (s, 1H), 7.37-7.41 (m, 4H), 7.14 (d, J=9.6 Hz, 1H), 7.09 (d, J=8.4 Hz, 2H), 6.72 (dd, J=8.8, 2.4 Hz, 1H), 6.44 (dd, J=3.2, 2.0 Hz, 1H), 6.25 (d, J=2.0 Hz, 1H), 4.92 (t, J=7.6 Hz, 1H), 4.52 (d, J=9.2 Hz, 1H), 4.42 (br t, J=8.2 Hz, 2H), 4.29 (br d, J=2.0 Hz, 1H), 4.01-4.07 (m, 2H), 3.56 (br d, J=4.4 Hz, 2H), 3.49 (br d, J=4.4 Hz, 4H), 3.24-3.30 (m, 3H), 3.15-3.23 (m, 3H), 3.07-3.11 (m, 2H), 2.98-3.04 (m, 2H), 2.85-2.94 (m, 2H), 2.69-2.74 (m, 2H), 2.46 (s, 3H), 2.31-2.35 (m, 3H), 2.20-2.27 (m, 2H), 2.09-2.17 (m, 2H), 2.02 (br s, 3H), 1.94-1.99 (m, 2H), 1.78-1.82 (m, 1H), 1.45-1.50 (m, 5H), 1.38 (d, J=7.2 Hz, 3H), 1.25 (br d, J=7.2 Hz, 2H), 0.94 (d, J=2.4 Hz, 15H) LC-MS (ESI⁺) m/z 1551.5. (M+H)⁺.

TABLE 5
Compounds synthesized via Method 3, using the corresponding
amines and acids for the coupling.

| | | | LCMS | |
|---|---|---|---|---|
| | | | (ESI+) | |
| | | | m/z | |
| I-#^{a} | Amine | Acid | (M + H)⁺ | ¹H NMR (400 MHz, DMSO-d6) |
| I-4 | LF | LG | 1479.6 | 8.98 (s, 1 H), 8.47 (d, J = 2.0 Hz, 1 |
| | | | | H), 8.37 (d, J = 7.6 Hz, 1 H), 8.13- |
| | | | | 8.24 (m, 1 H), 7.77-7.85 (m, 2 H), |
| | | | | 7.73 (d, J = 8.8 Hz, 2 H), 7.42-7.46 |
| | | | | (m, 6 H), 7.36-7.40 (m, 2 H), 7.29- |
| | | | | 7.33 (m, 2 H), 7.24 (br t, J = 7.6 |
| | | | | Hz, 2 H), 7.17 (d, J = 7.2 Hz, 1 H), |
| | | | | 7.10-7.19 (m, 2 H), 6.90-6.98 |
| | | | | (m, 2 H), 6.81 (br d, J = 8.8 Hz, 2 |
| | | | | H), 5.10 (d, J = 3.6 Hz, 1 H), 4.88- |
| | | | | 4.96 (m, 1 H), 4.52 (d, J = 9.6 Hz, 1 |
| | | | | H), 4.42 (t, J = 8.0 Hz, 1 H), 4.25- |
| | | | | 4.31 (m, 1 H), 4.04-4.12 (m, 1 H), |
| | | | | 3.61 (br d, J = 2.8 Hz, 6 H), 3.37 (br |
| | | | | s, 8 H), 3.16 (br s, 8 H), 2.63-2.71 |
| | | | | (m, 6 H), 2.45 (s, 3 H), 2.43 (br d, |
| | | | | J = 8.8 Hz, 4 H), 2.31-2.37 (m, 3 |
| | | | | H), 2.24 (br dd, J = 14.4, 7.2 Hz, 1 |
| | | | | H), 2.06-2.15 (m, 3 H), 1.96-2.04 |
| | | | | (m, 1 H), 1.75-1.84 (m, 1 H), 1.45- |
| | | | | 1.53 (m, 4 H), 1.37 (d, J = 7.2 Hz, |
| | | | | 3 H), 1.23-1.31 (m, 4 H), 0.92- |
| | | | | 0.97 (m, 9 H) |
| I-10 | LR | LP | 1424.5 | 9.97 (s, 1H), 8.99 (s, 1H), 8.46 (s, |
| | | | | 1H), 8.37 (d, J = 7.6 Hz, 1H), 8.28- |
| | | | | 8.11 (m, 1H), 7.80 (t, J = 9.6 Hz, |
| | | | | 2H), 7.75-7.69 (m, 3H), 7.67 (s, |
| | | | | 1H), 7.47-7.41 (m, 6H), 7.40- |
| | | | | 7.36 (m, 2H), 7.34-7.29 (m, 2H), |
| | | | | 7.25 (s, 2H), 7.17 (d, J = 7.2 Hz, |
| | | | | 2H), 6.90 (d, J = 9.6 Hz, 1H), 6.81 |
| | | | | (d, J = 8.8 Hz, 2H), 5.10 (d, J = 3.2 |
| | | | | Hz, 1H), 4.92 (t, J = 7.2 Hz, 1H), |
| | | | | 4.52 (d, J = 9.2 Hz, 1H), 4.43 (t, |
| | | | | J = 8.0 Hz, 1H), 4.28 (s, 1H), 4.13- |
| | | | | 4.02 (m, 1H), 3.60 (s, 2H), 3.20- |
| | | | | 3.09 (m, 6H), 2.55 (s, 6H), 2.46 (s, |
| | | | | 4H), 2.42 (s, 4H), 2.35-2.21 (m, |
| | | | | 5H), 2.17-1.97 (m, 5H), 1.84- |
| | | | | 1.75 (m, 1H), 1.59 (s, 2H), 1.51- |
| | | | | 1.44 (m, 2H), 1.37 (d, J = 7.2 Hz, |
| | | | | 3H), 1.29 (s, 7H), 0.93 (s, 9H) |
| I-15 | A | MQ | 1549.5 | 9.01 (d, J = 5.2 Hz, 1 H), 8.45- |
| | | | | 8.53 (m, 1 H), 8.38 (d, J = 7.6 Hz, |
| | | | | 1 H), 8.13 (s, 1 H), 7.96-8.00 (m, |
| | | | | 1 H), 7.81-7.87 (m, 3 H), 7.70- |
| | | | | 7.76 (m, 4 H), 7.49 (s, 4 H), 7.42 |
| | | | | (s, 2 H), 7.36-7.40 (m, 2 H), 7.26- |
| | | | | 7.32 (m, 3 H), 7.21 (t, J = 7.2 Hz, |
| | | | | 2 H), 7.12-7.17 (m, 1 H), 6.96- |
| | | | | 7.04 (m, 3 H), 6.79-6.86 (m, 2 H), |
| | | | | 5.10 (d, J = 3.2 Hz, 1 H), 4.88- |
| | | | | 4.95 (m, 1 H), 4.47-4.54 (m, 1 H), |
| | | | | 4.40-4.46 (m, 1 H), 4.24-4.31 |
| | | | | (m, 1 H), 4.07-4.17 (m, 1 H), 3.86- |
| | | | | 3.95 (m, 2 H), 3.55-3.64 (m, 2 |
| | | | | H), 3.40 (s, 2 H), 3.14-3.20 (m, 4 |
| | | | | H), 3.00-3.14 (m, 3 H), 2.78-2.87 |
| | | | | (m, 2 H), 2.70 (s, 6 H), 2.59-2.64 |
| | | | | (m, 1 H), 2.54 (s, 1 H), 2.43-2.47 |
| | | | | (m, 4 H), 2.36-2.42 (m, 4 H), 2.07- |
| | | | | 2.14 (m, 2 H), 1.98-2.04 (m, 1 |
| | | | | H), 1.74-1.82 (m, 2 H), 1.54-1.69 |
| | | | | (m, 3 H), 1.36-1.40 (m, 3 H), 0.92- |
| | | | | 0.96 (m, 9 H) |
| I-16 | MN | MK | 1477.6 | 8.99 (s, 1 H), 8.46 (d, J = 2.0 Hz, 1 |
| | | | | H), 8.37 (d, J = 7.6 Hz, 1 H), 8.20- |
| | | | | 8.26 (m, 1 H), 7.78-7.83 (m, 2 |
| | | | | H), 7.73 (d, J = 8.8 Hz, 2 H), 7.48 |
| | | | | (d, J = 1.2 Hz, 5 H), 7.42-7.45 (m, |
| | | | | 2 H), 7.37-7.40 (m, 2 H), 7.30- |
| | | | | 7.33 (m, 3 H), 7.25 (t, J = 7.6 Hz, |
| | | | | 2 H), 7.19 (dd, J = 9.2, 8.0 Hz, 2 |
| | | | | H), 6.90 (d, J = 9.6 Hz, 1 H), 6.80 |
| | | | | (d, J = 9.2 Hz, 2 H), 5.10 (d, J = |
| | | | | 3.6 Hz, 1 H), 4.92 (t, J = 7.2 Hz, 1 |
| | | | | H), 4.52 (dd, J = 9.6, 3.6 Hz, 1 H), |
| | | | | 4.43 (t, J = 7.6 Hz, 1 H), 4.28 (s, 1 |
| | | | | H), 4.07 (d, J = 5.2 Hz, 1 H), 3.77 |
| | | | | (ddd, J = 6.4, 3.2, 1.6 Hz, 2 H), |
| | | | | 3.60 (s, 3 H), 3.47 (br d, J = 11.2 |
| | | | | Hz, 1 H), 3.38 (s, 3 H), 3.14 (s, 5 |
| | | | | H), 2.81-2.94 (m, 3 H), 2.55 (d, |
| | | | | J = 2.4 Hz, 6 H), 2.46 (s, 3 H), 2.40 |
| | | | | (s, 4 H), 2.27 (dd, J = 13.6, 6.4 Hz, |
| | | | | 2 H), 2.08-2.17 (m, 3 H), 1.98- |
| | | | | 2.04 (m, 2 H), 1.90 (d, J = 9.2 Hz, |
| | | | | 1 H), 1.75-1.87 (m, 2 H), 1.66- |
| | | | | 1.75 (m, 1 H), 1.57-1.65 (m, 1 H), |
| | | | | 1.43-1.53 (m, 5 H), 1.37 (d, J = |
| | | | | 6.4 Hz, 3 H), 1.24 (s, 1 H), 0.93 (d, |
| | | | | J = 7.2 Hz, 9 H) |
| I-26 | ND | MZ | 1584.6 | 8.99 (s, 1H), 8.42-8.32 (m, 2H), |
| | | | | 8.08 (s, 1H), 7.96 (d, J = 9.2 Hz, |
| | | | | 1H), 7.78 (d, J = 9.2 Hz, 1H), 7.67 |
| | | | | (d, J = 8.8 Hz, 2H), 7.45 (br d, J = |
| | | | | 18.0 Hz, 5H), 7.40-7.27 (m, 8H), |
| | | | | 7.22-7.16 (m, 2H), 7.08 (d, J = 7.6 |
| | | | | Hz, 1H), 6.93-6.87 (m, 1H), 6.73 |
| | | | | (d, J = 8.8 Hz, 3H), 5.22 (d, J = 4.0 |
| | | | | Hz, 1H), 5.10 (d, J = 3.6 Hz, 1H), |
| | | | | 4.96-4.89 (m, 1H), 4.52 (d, J = 9.2 |
| | | | | Hz, 1H), 4.43 (t, J = 8.0 Hz, 1H), |
| | | | | 4.34-4.26 (m, 4H), 4.02 (dd, J = |
| | | | | 4.0, 5.6 Hz, 1H), 3.78-3.69 (m, |
| | | | | 1H), 3.67-3.52 (m, 5H), 3.28- |
| | | | | 3.24 (m, 2H), 2.66-2.55 (m, 3H), |
| | | | | 2.46 (s, 3H), 2.43 (s, 2H), 2.30- |
| | | | | 2.17 (m, 2H), 2.16-2.04 (m, 4H), |
| | | | | 2.01 (d, J = 1.2 Hz, 2H), 1.93-1.88 |
| | | | | (m, 1H), 1.80 (s, 1H), 1.62-1.41 |
| | | | | (m, 6H), 1.38 (d, J = 7.2 Hz, 3H), |
| | | | | 1.25 (s, 9H), 1.14-1.08 (m, 1H), |
| | | | | 1.00 (d, J = 2.8 Hz, 1H), 0.94 (s, |
| | | | | 9H), 0.85 (d, J = 2.0 Hz, 1H) |
| I-34 | LR | LG | 1410.5 | 9.97 (s, 1H), 8.99 (s, 1H), 8.47 (d, |
| | | | | J = 1.6 Hz, 1H), 8.37 (d, J = 8.0 |
| | | | | Hz, 1H), 7.81 (t, J = 9.6 Hz, 2H), |
| | | | | 7.75-7.66 (m, 5H), 7.46-7.42 (m, |
| | | | | 6H), 7.39-7.36 (m, 2H), 7.33- |
| | | | | 7.30 (m, 2H), 7.25 (t, J = 7.6 Hz, |
| | | | | 2H), 7.18 (dd, J = 3.6, 7.6 Hz, 2H), |
| | | | | 6.92 (d, J = 9.2 Hz, 1H), 6.81 (d, |
| | | | | J = 8.4 Hz, 2H), 5.10 (d, J = 3.6 Hz, |
| | | | | 1H), 4.92 (t, J = 7.2 Hz, 1H), 4.52 |
| | | | | (d, J = 9.2 Hz, 1H), 4.43 (t, J = 8.0 |
| | | | | Hz, 1H), 4.28 (s, 1H), 4.11-4.03 |
| | | | | (m, 1H), 3.61 (s, 2H), 3.30 (s, 3H), |
| | | | | 3.16 (s, 4H), 3.05-2.91 (m, 2H), |
| | | | | 2.62 (s, 6H), 2.46 (s, 3H), 2.42 (d, |
| | | | | J = 2.8 Hz, 4H), 2.33-2.28 (m, |
| | | | | 3H), 2.26-2.22 (m, 1H), 2.16- |
| | | | | 1.99 (m, 5H), 1.80 (ddd, J = 4.4, |
| | | | | 8.0, 12.8 Hz, 1H), 1.62-1.56 (m, |
| | | | | 2H), 1.52-1.46 (m, 2H), 1.37 (d, |
| | | | | J = 6.8 Hz, 3H), 1.30 (d, J = 2.8 Hz, |
| | | | | 4H), 0.94 (s, 9H) |
| I-47 | NP | KT | 1349.5 | 9.03-8.91 (m, 1H), 8.37 (d, J = 7.6 |
| | | | | Hz, 1H), 8.17-8.11 (m, 1H), 8.07 |
| | | | | (d, J = 2.0 Hz, 1H), 7.97-7.91 (m, |
| | | | | 1H), 7.79 (d, J = 9.2 Hz, 1H), 7.75 |
| | | | | (d, J = 8.8 Hz, 2H), 7.45-7.42 (m, |
| | | | | 2H), 7.40-7.35 (m, 4H), 7.33- |
| | | | | 7.28 (m, 2H), 7.23-7.18 (m, 1H), |
| | | | | 6.89-6.81 (m, 1H), 6.71 (d, J = 8.8 |
| | | | | Hz, 1H), 6.57 (d, J = 8.8 Hz, 2H), |
| | | | | 5.15-5.05 (m, 1H), 4.96-4.88 (m, |
| | | | | 1H), 4.51 (d, J = 9.2 Hz, 1H), 4.43 |
| | | | | (t, J = 8.0 Hz, 1H), 4.28 (s, 1H), |
| | | | | 4.05-3.97 (m, 1H), 3.56 (s, 3H), |
| | | | | 3.15-2.95 (m, 5H), 2.67-2.64 (m, |
| | | | | 2H), 2.46 (s, 3H), 2.45-1.88 (m, |
| | | | | 18H), 1.88-1.57 (m, 3H), 1.56- |
| | | | | 1.33 (m, 9H), 1.32-1.14 (m, 3H), |
| | | | | 1.01-0.88 (m, 11H) |
| I-169 | RG | LG | 1664.7 | 11.67 (br s, 1H), 8.99 (s, 1H), 8.37 |
| | | | | (d, J = 7.6 Hz, 1H), 8.12 (br s, 1H), |
| | | | | 8.02 (d, J = 2.4 Hz, 1H), 7.92-7.85 |
| | | | | (m, 1H), 7.78 (br d, J = 9.2 Hz, |
| | | | | 1H), 7.56-7.47 (m, 3H), 7.46- |
| | | | | 7.42 (m, 2H), 7.42-7.31 (m, 5H), |
| | | | | 7.05 (br d, J = 8.4 Hz, 2H), 7.01- |
| | | | | 6.93 (m, 1H), 6.67 (br d, J = 8.4 |
| | | | | Hz, 1H), 6.39 (br s, 1H), 6.20 (br s, |
| | | | | 1H), 5.10 (d, J = 3.6 Hz, 1H), 4.92 |
| | | | | (br t, J = 7.2 Hz, 1H), 4.52 (d, J = |
| | | | | 9.2 Hz, 1H), 4.43 (t, J = 8.0 Hz, |
| | | | | 1H), 4.32-4.25 (m, 1H), 3.59 (br |
| | | | | d, J = 5.2 Hz, 9H), 3.43 (br s, 6H), |
| | | | | 3.06 (br d, J = 1.6 Hz, 4H), 2.77- |
| | | | | 2.65 (m, 4H), 2.46 (s, 3H), 2.41 (br |
| | | | | d, J = 2.0 Hz, 4H), 2.31-2.19 (m, |
| | | | | 9H), 2.15-2.08 (m, 2H), 2.06- |
| | | | | 1.94 (m, 3H), 1.80 (dt, J = 4.0, 8.6 |
| | | | | Hz, 1H), 1.76-1.69 (m, 3H), 1.52- |
| | | | | 1.41 (m, 5H), 1.38 (d, J = 6.8 Hz, |
| | | | | 3H), 1.30-1.21 (m, 8H), 0.94 (s, |
| | | | | 9H) |
| I-171 | RL | LG | 1608.7 | 11.65-11.50 (m, 1H), 8.98 (s, 1H), |
| | | | | 8.38 (d, J = 7.6 Hz, 1H), 8.10-7.99 |
| | | | | (m, 1H), 7.95 (d, J = 2.4 Hz, 1H), |
| | | | | 7.86-7.75 (m, 2H), 7.56 (d, J = 8.4 |
| | | | | Hz, 1H), 7.47-7.31 (m, 9H), 7.09 |
| | | | | (br d, J = 8.4 Hz, 2H), 6.87-6.78 |
| | | | | (m, 1H), 6.67-6.60 (m, 1H), 6.38- |
| | | | | 6.30 (m, 1H), 6.23 (s, 1H), 5.10 |
| | | | | (d, J = 3.4 Hz, 1H), 5.01-4.85 (m, |
| | | | | 1H), 4.52 (d, J = 9.6 Hz, 1H), 4.43 |
| | | | | (t, J = 8.0 Hz, 1H), 4.28 (br s, 1H), |
| | | | | 3.67-3.49 (m, 3H), 3.01 (br s, |
| | | | | 4H), 2.88-2.78 (m, 2H), 2.74- |
| | | | | 2.65 (m, 3H), 2.56 (br s, 6H), 2.46 |
| | | | | (s, 6H), 2.42-2.32 (m, 3H), 2.28- |
| | | | | 2.11 (m, 13H), 2.02 (br d, J = 12.0 |
| | | | | Hz, 2H), 1.89-1.77 (m, 4H), 1.59- |
| | | | | 1.43 (m, 6H), 1.38 (br d, J = 7.2 |
| | | | | Hz, 3H), 1.25 (br s, 5H), 0.94 (s, |
| | | | | 9H), 0.90 (s, 3H) |
| I-189 | RL | SR | 1551.7 | 11.57 (br s, 1H), 8.35 (br d, J = 8.4 |
| | | | | Hz, 1H), 8.15 (s, 1H), 8.03 (s, 1H), |
| | | | | 7.95 (d, J = 2.4 Hz, 1H), 7.81 (br |
| | | | | d, J = 9.2 Hz, 2H), 7.57 (d, J = 8.8 |
| | | | | Hz, 1H), 7.45 (br d, J = 2.4 Hz, |
| | | | | 1H), 7.41 (d, J = 8.0 Hz, 2H), 7.37- |
| | | | | 7.24 (m, 6H), 7.09 (br d, J = 8.4 |
| | | | | Hz, 2H), 6.81 (br d, J = 9.2 Hz, |
| | | | | 1H), 6.63 (br d, J = 8.8 Hz, 1H), |
| | | | | 6.33 (br s, 1H), 6.24 (s, 1H), 5.11 |
| | | | | (br s, 1H), 4.87-4.80 (m, 1H), |
| | | | | 4.78-4.70 (m, 1H), 4.52 (d, J = 9.2 |
| | | | | Hz, 1H), 4.45 (brt, J = 8.0 Hz, 1H), |
| | | | | 4.27 (br s, 1H), 4.13 (s, 1H), 3.61 |
| | | | | (br s, 2H), 3.57 (br d, J = 5.2 Hz, |
| | | | | 2H), 3.43-3.40 (m, 4H), 3.01 (br |
| | | | | s, 4H), 2.78 (br s, 2H), 2.74-2.64 |
| | | | | (m, 3H), 2.53 (br s, 4H), 2.48- |
| | | | | 2.38 (m, 6H), 2.27-2.10 (m, 13H), |
| | | | | 2.06-1.95 (m, 2H), 1.90-1.76 (m, |
| | | | | 4H), 1.55-1.40 (m, 6H), 1.24 (br |
| | | | | s, 4H), 0.93 (s, 9H), 0.90 (s, 3H) |
| I-191 | ST | SR | 1607.7 | 11.73-11.61 (m, 1H), 8.39-8.27 |
| | | | | (m, 1H), 8.17-8.09 (m, 1H), 8.05- |
| | | | | 8.00 (m, 1H), 7.93-7.86 (m, 1H), |
| | | | | 7.79 (br d, J = 8.8 Hz, 1H), 7.54- |
| | | | | 7.48 (m, 3H), 7.42 (d, J = 8.0 Hz, |
| | | | | 2H), 7.38-7.29 (m, 5H), 7.08 (br |
| | | | | d, J = 8.0 Hz, 2H), 6.71-6.63 (m, |
| | | | | 1H), 6.46-6.35 (m, 1H), 6.24- |
| | | | | 6.14 (m, 1H), 5.14-5.06 (m, 1H), |
| | | | | 4.88-4.79 (m, 1H), 4.71 (s, 1H), |
| | | | | 4.56-4.49 (m, 1H), 4.49-4.41 (m, |
| | | | | 1H), 4.31-4.24 (m, 1H), 4.13 (s, |
| | | | | 1H), 3.64-3.55 (m, 9H), 3.49- |
| | | | | 3.42 (m, 6H), 3.08-3.02 (m, 4H), |
| | | | | 2.68 (br d, J = 2.0 Hz, 4H), 2.36- |
| | | | | 2.32 (m, 3H), 2.27-2.14 (m, 11H), |
| | | | | 2.04-1.98 (m, 2H), 1.88-1.67 (m, |
| | | | | 8H), 1.53-1.40 (m, 7H), 1.32- |
| | | | | 1.18 (m, 6H), 0.93 (s, 9H), 0.87 (br |
| | | | | s, 3H) |
| I-206 | A | SV | 1568.7 | 8.99-8.96 (m, 1H), 8.36 (d, J = 7.6 |
| | | | | Hz, 1H), 8.05 (d, J = 2.0 Hz, 1H), |
| | | | | 7.91 (dd, J = 2.4, 8.9 Hz, 1H), 7.79- |
| | | | | 7.74 (m, 1H), 7.70 (d, J = 8.8 Hz, |
| | | | | 2H), 7.45-7.42 (m, 2H), 7.38 (d, |
| | | | | J = 1.6 Hz, 2H), 7.37 (d, J = 1.6 Hz, |
| | | | | 1H), 7.36 (d, J = 2.0 Hz, 2H), 7.35 |
| | | | | (br s, 1H), 7.32-7.28 (m, 2H), |
| | | | | 7.21 (d, J = 7.2 Hz, 1H), 7.13-7.10 |
| | | | | (m, 2H), 6.81 (d, J = 9.2 Hz, 1H), |
| | | | | 6.76 (d, J = 8.8 Hz, 2H), 6.68 (br |
| | | | | d, J = 8.4 Hz, 1H), 5.07 (d, J = 3.6 |
| | | | | Hz, 1H), 4.94-4.89 (m, 1H), 4.51 |
| | | | | (d, J = 9.2 Hz, 1H), 4.45-4.40 (m, |
| | | | | 1H), 4.27 (br d, J = 2.4 Hz, 1H), |
| | | | | 4.03-3.97 (m, 1H), 3.61 (br d, J = |
| | | | | 2.4 Hz, 2H), 3.44-3.36 (m, 3H), |
| | | | | 3.11 (br s, 5H), 2.73 (s, 2H), 2.67 |
| | | | | (td, J = 1.6, 3.7 Hz, 1H), 2.52 (d, |
| | | | | J = 1.6 Hz, 4H), 2.45 (s, 4H), 2.32 |
| | | | | (br dd, J = 1.6, 3.6 Hz, 2H), 2.30- |
| | | | | 2.20 (m, 12H), 2.10 (br d, J = 4.0 |
| | | | | Hz, 2H), 1.99 (br s, 3H), 1.69- |
| | | | | 1.58 (m, 6H), 1.47-1.40 (m, 3H), |
| | | | | 1.37 (d, J = 7.2 Hz, 3H), 1.34-1.22 |
| | | | | (m, 3H), 0.95 (d, J = 11.2 Hz, 13H) |
| I-231 | A | DN | 1076.7 | 8.98 (s, 1H), 8.36 (br d, J = 8.0 Hz, |
| | | | | 1H), 8.18-8.07 (m, 1H), 7.91- |
| | | | | 7.81 (m, 3H), 7.71 (br d, J = 8.4 |
| | | | | Hz, 2H), 7.45-7.35 (m, 9H), 7.11 |
| | | | | (br d, J = 8.4 Hz, 2H), 6.87 (br d, |
| | | | | J = 8.8 Hz, 2H), 5.11 (br d, J = 2.8 |
| | | | | Hz, 1H), 4.91 (brt, J = 7.2 Hz, 1H), |
| | | | | 4.51-4.39 (m, 2H), 4.27 (br s, |
| | | | | 1H), 3.59 (br s, 2H), 3.23 (br s, |
| | | | | 3H), 2.91-2.84 (m, 2H), 2.78 (br |
| | | | | s, 2H), 2.45 (s, 3H), 2.29 (br s, 4H), |
| | | | | 2.22 (br s, 3H), 1.99 (br s, 3H), |
| | | | | 1.82-1.74 (m, 1H), 1.43 (br t, J = |
| | | | | 5.6 Hz, 3H), 1.36 (br d, J = 7.2 Hz, |
| | | | | 3H), 1.24 (br s, 1H), 0.96 (s, 6H), |
| | | | | 0.81 (s, 9H) |
| I-238 | TS | TP | 1529.5 | 8.98 (s, 1H), 8.53 (d, J = 4.4 Hz, |
| | | | | 1H), 8.13 (d, J = 1.6 Hz, 1H), 8.07 |
| | | | | (s, 1H), 7.94 (d, J = 9.6 Hz, 1H), |
| | | | | 7.70 (d, J = 7.8 Hz, 2H), 7.44 (d, |
| | | | | J = 8.4 Hz, 1H), 7.39-7.32 (m, 4H), |
| | | | | 7.31-7.22 (m, 5H), 7.22-7.15 (m, |
| | | | | 1H), 7.11 (d, J = 6.8 Hz, 2H), 6.92- |
| | | | | 6.84 (m, 1H), 6.77 (d, J = 8.0 Hz, |
| | | | | 2H), 6.73-6.66 (m, 1H), 5.16 (s, |
| | | | | 1H), 4.59 (d, J = 8.8 Hz, 1H), 4.53- |
| | | | | 4.46 (m, 1H), 4.45-4.24 (m, 3H), |
| | | | | 4.08-3.93 (m, 1H), 3.70-3.56 (m, |
| | | | | 2H), 3.23 (d, J = 3.2 Hz, 2H), 3.12 |
| | | | | (s, 4H), 2.73 (d, J = 8.8 Hz, 2H), |
| | | | | 2.63 (s, 2H), 2.45 (s, 4H), 2.32- |
| | | | | 2.14 (m, 9H), 2.13-2.03 (m, 2H), |
| | | | | 1.98 (s, 3H), 1.96-1.62 (m, 6H), |
| | | | | 1.51-1.30 (m, 5H), 1.21 (d, J = 8.8 |
| | | | | Hz, 4H), 0.96 (s, 17H) |
| I-239^{b} | TY | TW | 828.9 | 8.98 (s, 1H), 8.38 (br d, J = 8.0 Hz, |
| | | | | 1H), 8.16 (br s, 1H), 7.98 (br d, J = |
| | | | | 8.8 Hz, 1H), 7.81-7.73 (m, 3H), |
| | | | | 7.44-7.36 (m, 6H), 7.32-7.29 (m, |
| | | | | 2H), 7.24 (br t, J = 7.6 Hz, 2H), |
| | | | | 7.16 (brt, J = 7.6 Hz, 2H), 7.09 (br |
| | | | | d, J = 8.4 Hz, 2H), 7.00-6.90 (m, |
| | | | | 3H), 5.10 (br d, J = 2.4 Hz, 1H), |
| | | | | 4.91 (brt, J = 6.8 Hz, 1H), 4.51 (br |
| | | | | d, J = 9.6 Hz, 1H), 4.41 (br t, J = |
| | | | | 8.0 Hz, 1H), 4.27 (br s, 1H), 4.16- |
| | | | | 4.12 (m, 1H), 3.93 (dt, J = 4.0, 6.4 |
| | | | | Hz, 2H), 3.61 (br d, J = 10.4 Hz, |
| | | | | 14H), 3.46 (br s, 7H), 2.45 (s, 5H), |
| | | | | 2.33-2.22 (m, 10H), 2.15-1.97 |
| | | | | (m, 8H), 1.82-1.76 (m, 2H), 1.50- |
| | | | | 1.44 (m, 5H), 1.37 (br d, J = 6.8 |
| | | | | Hz, 6H), 1.29-1.22 (m, 6H), 0.93 |
| | | | | (s, 9H) |
| I-247 | UP | LP | 1595.6 | 11.66 (s, 1H), 8.98 (s, 1H), 8.38 (d, |
| | | | | J = 7.6 Hz, 1H), 8.10 (s, 1H), 8.01 |
| | | | | (d, J = 2.4 Hz, 1H), 7.87 (d, J = 8.8 |
| | | | | Hz, 1H), 7.77 (d, J = 9.2 Hz, 1H), |
| | | | | 7.68 (t, J = 6.0 Hz, 1H), 7.53-7.46 |
| | | | | (m, 3H), 7.45-7.42 (m, 2H), 7.39 |
| | | | | (s, 1H), 7.38-7.33 (m, 3H), 7.31- |
| | | | | 7.24 (m, 1H), 7.08 (d, J = 8.4 Hz, |
| | | | | 2H), 7.00-6.89 (m, 1H), 6.71- |
| | | | | 6.61 (m, 1H), 6.38 (s, 1H), 6.20 (s, |
| | | | | 1H), 5.10 (d, J = 3.6 Hz, 1H), 4.97- |
| | | | | 4.88 (m, 1H), 4.52 (d, J = 9.2 Hz, |
| | | | | 1H), 4.43 (t, J = 8.0 Hz, 1H), 4.28 |
| | | | | (d, J = 1.6 Hz, 1H), 3.62-3.57 (m, |
| | | | | 6H), 3.07-3.00 (m, 5H), 2.93 (dd, |
| | | | | J = 5.6, 12.8 Hz, 1H), 2.69 (s, 2H), |
| | | | | 2.46-2.45 (m, 3H), 2.40 (d, J = 1.2 |
| | | | | Hz, 6H), 2.25-2.15 (m, 7H), 2.13- |
| | | | | 2.07 (m, 4H), 2.06-1.97 (m, 2H), |
| | | | | 1.92 (d, J = 16.0 Hz, 2H), 1.80 (dt, |
| | | | | J = 4.4, 8.5 Hz, 1H), 1.75-1.68 |
| | | | | (m, 2H), 1.46 (d, J = 6.8 Hz, 5H), |
| | | | | 1.38 (d, J = 6.8 Hz, 3H), 1.22 (s, |
| | | | | 8H), 0.93 (s, 9H), 0.86 (s, 3H) |
| I-253 | WD | KR | 1550.6 | 8.99 (s, 1H), 8.38-8.36 (d, J = 8.0 |
| | | | | Hz, 1H), 8.11 (s, 1H), 7.82 (s, 1H), |
| | | | | 7.68 (s, 1H), 7.66 (s, 2H), 7.48- |
| | | | | 7.46 (d, J = 8.0 Hz, 1H), 7.45 (s, |
| | | | | 2H), 7.42-7.38 (m, 3H), 7.35-7.33 |
| | | | | (m, 10H), 7.36 (br d, J = 8.4 Hz, |
| | | | | 1H), 7.16 (br d, J = 7.6 Hz, 1H), |
| | | | | 6.75 (d, J = 8.0 Hz, 1H), 6.72 (d, |
| | | | | J = 7.6 Hz, 1H), 5.50 (s, 1H), 5.22 (s, |
| | | | | 1H), 5.21-5.09 (m, 1H), 4.91 (s, |
| | | | | 1H), 4.52-4.29 (m, 4H), 4.08(m, |
| | | | | 1H), 3.77-3.48 (m, 10H), 2.61- |
| | | | | 2.58 (br s, 4H), 2.46 (s, 3H), 2.07- |
| | | | | 2.0 (m, 8 H), 1.90-1.78 (m, 4 |
| | | | | H), 1.47-1.36 (m, 11H), 0.98-0.93 |
| | | | | (m, 3H), 0.86 (s, 9H), 0.81 (br d, |
| | | | | J = 7.5 Hz, 1H) |
| I-256 | NV | VG | 1607.7 | 11.69 (br s, 1H), 8.37 (d, J = 8.0 |
| | | | | Hz, 1H), 8.13 (s, 1H), 8.03 (d, J = |
| | | | | 2.4 Hz, 1H), 7.89 (br d, J = 9.2 Hz, |
| | | | | 1H), 7.85-7.77 (m, 1H), 7.55- |
| | | | | 7.46 (m, 3H), 7.44-7.39 (m, 2H), |
| | | | | 7.33 (dd, J = 8.4, 16.4 Hz, 5H), |
| | | | | 7.08 (d, J = 8.4 Hz, 2H), 7.00 (br |
| | | | | d, J = 9.2 Hz, 1H), 6.67 (br d, J = |
| | | | | 8.8 Hz, 1H), 6.42-6.36 (m, 1H), |
| | | | | 6.19 (s, 1H), 5.12 (d, J = 3.6 Hz, |
| | | | | 1H), 4.87-4.79 (m, 1H), 4.76 (t, |
| | | | | J = 6.0 Hz, 1H), 4.66 (s, 1H), 4.52 |
| | | | | (d, J = 9.2 Hz, 1H), 4.44 (t, J = 8.0 |
| | | | | Hz, 1H), 4.27 (br d, J = 2.4 Hz, |
| | | | | 1H), 4.14 (s, 1H), 3.66-3.52 (m, |
| | | | | 9H), 3.44-3.40 (m, 4H), 3.05 (br |
| | | | | s, 4H), 2.74-2.65 (m, 2H), 2.48- |
| | | | | 2.32 (m, 10H), 2.29-2.10 (m, |
| | | | | 13H), 2.05-1.96 (m, 2H), 1.94- |
| | | | | 1.69 (m, 5H), 1.53-1.38 (m, 6H), |
| | | | | 1.24 (br s, 6H), 0.93 (s, 9H), 0.90 |
| | | | | (s, 3H) |
| I-257 | NV | RW | 1579.7 | 11.67 (br d, J = 0.8 Hz, 1H), 8.36 |
| | | | | (d, J = 8.0 Hz, 1H), 8.11 (s, 1H), |
| | | | | 8.01 (d, J = 2.4 Hz, 1H), 7.90-7.79 |
| | | | | (m, 2H), 7.49 (br dd, J = 2.8, 6.0 |
| | | | | Hz, 3H), 7.41 (d, J = 8.0 Hz, 2H), |
| | | | | 7.32 (dd, J = 8.4, 16.4 Hz, 5H), |
| | | | | 7.08 (d, J = 8.4 Hz, 2H), 7.01-6.94 |
| | | | | (m, 1H), 6.66 (br d, J = 8.8 Hz, |
| | | | | 1H), 6.38 (br s, 1H), 6.19 (s, 1H), |
| | | | | 5.11 (d, J = 3.6 Hz, 1H), 4.87-4.79 |
| | | | | (m, 1H), 4.75 (t, J = 6.0 Hz, 1H), |
| | | | | 4.50 (d, J = 9.2 Hz, 1H), 4.43 (t, |
| | | | | J = 8.0 Hz, 1H), 4.26 (br s, 1H), 4.14 |
| | | | | (s, 1H), 3.62-3.51 (m, 9H), 3.43- |
| | | | | 3.39 (m, 4H), 3.03 (br s, 4H), 2.68 |
| | | | | (br dd, J = 3.6, 4.8 Hz, 2H), 2.45- |
| | | | | 2.31 (m, 10H), 2.26-2.10 (m, |
| | | | | 13H), 2.04-1.96 (m, 2H), 1.90- |
| | | | | 1.68 (m, 5H), 1.47 (dt, J = 7.2, 14.8 |
| | | | | Hz, 6H), 1.27-1.20 (m, 2H), 0.92 |
| | | | | (s, 9H), 0.89 (s, 3H) |
| I-258 | NV | UX | 1724.7 | 11.84-11.77 (m, 1H), 11.77- |
| | | | | 11.76 (m, 1H), 9.12-8.91 (m, 1H), |
| | | | | 8.53 (s, 1H), 8.23-8.16 (m, 1H), |
| | | | | 8.07 (d, J = 2.3 Hz, 1H), 7.98-7.94 |
| | | | | (m, 1H), 7.64-7.59 (m, 1H), 7.58- |
| | | | | 7.53 (m, 1H), 7.53-7.47 (m, 2H), |
| | | | | 7.43-7.38 (m, 2H), 7.29-7.22 (m, |
| | | | | 2H), 7.19-7.13 (m, 3H), 7.04- |
| | | | | 6.98 (m, 2H), 6.76-6.69 (m, 1H), |
| | | | | 6.48-6.41 (m, 1H), 6.28-6.17 (m, |
| | | | | 1H), 5.23-5.07 (m, 1H), 4.57 (br |
| | | | | d, J = 9.6 Hz, 1H), 4.51 (br t, J = |
| | | | | 8.0 Hz, 1H), 4.34-4.19 (m, 3H), |
| | | | | 4.13-3.99 (m, 5H), 3.90-3.90 (m, |
| | | | | 7H), 3.96-3.89 (m, 4H), 3.40 (br |
| | | | | d, J = 12.4 Hz, 3H), 3.20-3.16 (m, |
| | | | | 3H), 3.14-3.03 (m, 7H), 2.77- |
| | | | | 2.63 (m, 3H), 2.48-2.46 (m, 3H), |
| | | | | 2.42-2.25 (m, 6H), 2.16-2.08 (m, |
| | | | | 1H), 1.97 (br d, J = 7.6 Hz, 2H), |
| | | | | 1.82-1.72 (m, 4H), 1.60-1.44 (m, |
| | | | | 6H), 1.41-1.29 (m, 8H), 1.26- |
| | | | | 1.14 (m, 6H), 0.96 (s, 9H) |
| I-259 | UZ | SR | 1605.7 | 11.72-11.61 (m, 1H), 8.42-8.23 |
| | | | | (m, 1H), 8.18-8.07 (m, 1H), 8.05- |
| | | | | 7.98 (m, 1H), 7.93-7.85 (m, 1H), |
| | | | | 7.82-7.75 (m, 1H), 7.52-7.46 (m, |
| | | | | 3H), 7.41 (d, J = 8.0 Hz, 2H), 7.37- |
| | | | | 7.28 (m, 5H), 7.08 (br d, J = 8.0 |
| | | | | Hz, 2H), 7.04-6.92 (m, 1H), 6.69- |
| | | | | 6.62 (m, 1H), 6.42-6.36 (m, 1H), |
| | | | | 6.22-6.16 (m, 1H), 5.12-5.05 (m, |
| | | | | 1H), 4.88-4.78 (m, 1H), 4.76- |
| | | | | 4.68 (m, 1H), 4.56-4.39 (m, 3H), |
| | | | | 4.26 (br s, 1H), 4.15-4.10 (m, |
| | | | | 1H), 3.65-3.53 (m, 9H), 3.03 (br |
| | | | | s, 4H), 2.75-2.64 (m, 4H), 2.45- |
| | | | | 2.34 (m, 7H), 2.29-1.94 (m, 16H), |
| | | | | 1.88-1.68 (m, 6H), 1.52-1.41 (m, |
| | | | | 6H), 1.31-1.20 (m, 5H), 0.92 (s, |
| | | | | 9H), 0.88-0.82 (m, 3H) |
| I-261 | NV | VA | 1621.7 | 11.74 (br d, J = 2.0 Hz, 1H), 8.40- |
| | | | | 8.33 (m, 1H), 8.14-8.08 (m, 1H), |
| | | | | 8.06-8.00 (m, 1H), 7.90-7.78 (m, |
| | | | | 2H), 7.52-7.46 (m, 3H), 7.43- |
| | | | | 7.39 (m, 2H), 7.37-7.27 (m, 6H), |
| | | | | 7.09 (d, J = 8.4 Hz, 2H), 7.01-6.93 |
| | | | | (m, 1H), 6.70-6.63 (m, 1H), 6.38 |
| | | | | (br d, J = 1.6 Hz, 1H), 6.20 (s, 1H), |
| | | | | 5.11 (d, J = 3.6 Hz, 1H), 4.86-4.79 |
| | | | | (m, 1H), 4.75 (t, J = 5.6 Hz, 1H), |
| | | | | 4.55-4.49 (m, 1H), 4.48-4.42 (m, |
| | | | | 1H), 4.32-4.23 (m, 1H), 4.14 (s, |
| | | | | 1H), 3.65-3.51 (m, 10H), 3.04 (br |
| | | | | s, 4H), 2.75-2.61 (m, 3H), 2.30- |
| | | | | 2.07 (m, 18H), 1.92-1.68 (m, 6H), |
| | | | | 1.54-1.35 (m, 8H), 1.24 (br s, |
| | | | | 10H), 0.97-0.88 (m, 15H) |
| I-263 | VF | SR | 1508.6 | 11.63-11.56 (m, 1H), 8.37-8.29 |
| | | | | (m, 2H), 8.15-8.07 (m, 1H), 7.99- |
| | | | | 7.94 (m, 1H), 7.80 (d, J = 9.2 Hz, |
| | | | | 1H), 7.54 (d, J = 9.2 Hz, 1H), 7.46 |
| | | | | (s, 1H), 7.41 (d, J = 8.4 Hz, 2H), |
| | | | | 7.35 (d, J = 8.4 Hz, 3H), 7.31 (d, |
| | | | | J = 8.4 Hz, 2H), 7.06 (d, J = 8.4 Hz, |
| | | | | 2H), 6.67-6.61 (m, 1H), 6.37- |
| | | | | 6.22 (m, 2H), 5.14-5.07 (m, 1H), |
| | | | | 4.83 (q, J = 6.4 Hz, 1H), 4.75 (t, |
| | | | | J = 5.6 Hz, 1H), 4.52 (d, J = 9.6 Hz, |
| | | | | 1H), 4.45 (t, J = 8.4 Hz, 1H), 4.27 |
| | | | | (s, 3H), 4.12 (s, 1H), 3.61 (s, 2H), |
| | | | | 3.56 (dd, J = 6.4, 12.4 Hz, 3H), |
| | | | | 3.05 (d, J = 1.2 Hz, 5H), 2.82-2.74 |
| | | | | (m, 2H), 2.30-2.21 (m, 7H), 2.18- |
| | | | | 2.12 (m, 3H), 2.03-1.91 (m, 6H), |
| | | | | 1.84-1.74 (m, 1H), 1.51-1.43 (m, |
| | | | | 4H), 1.41-1.38 (m, 2H), 1.34- |
| | | | | 1.19 (m, 6H), 0.93 (s, 17H) |
| I-269 | NV | VM | 1594.7 | 11.66 (s, 1H), 8.43 (d, J = 8.0 Hz, |
| | | | | 1H), 8.15-8.09 (m, 1H), 8.02 (d, |
| | | | | J = 1.6 Hz, 1H), 7.91-7.84 (m, 1H), |
| | | | | 7.81-7.76 (m, 3H), 7.50 (d, J = 8.0 |
| | | | | Hz, 5H), 7.38-7.27 (m, 3H), 7.09 |
| | | | | (d, J = 8.0 Hz, 2H), 7.01-6.93 (m, |
| | | | | 1H), 6.70-6.63 (m, 1H), 6.38 (s, |
| | | | | 1H), 6.20 (s, 1H), 5.11 (d, J = 3.6 |
| | | | | Hz, 1H), 4.92-4.87 (m, 1H), 4.83 |
| | | | | (t, J = 5.6 Hz, 1H), 4.52 (d, J = 9.6 |
| | | | | Hz, 1H), 4.45 (t, J = 8.0 Hz, 1H), |
| | | | | 4.31-4.23 (m, 1H), 3.60 (d, J = 4.0 |
| | | | | Hz, 8H), 3.42-3.38 (m, 4H), 3.04 |
| | | | | (s, 4H), 2.74-2.67 (m, 2H), 2.47- |
| | | | | 2.46 (m, 2H), 2.41 (s, 6H), 2.28- |
| | | | | 2.22 (m, 4H), 2.19 (s, 8H), 2.13- |
| | | | | 2.08 (m, 2H), 2.05-1.98 (m, 2H), |
| | | | | 1.90-1.82 (m, 1H), 1.80-1.70 (m, |
| | | | | 3H), 1.58-1.35 (m, 8H), 1.25 (d, |
| | | | | J = 2.8 Hz, 5H), 0.93 (s, 9H), 0.90 |
| | | | | (s, 3H) |

^{a}The reaction was run under standard conditions for 2-4 hrs from 25-30° C. HOAt could also be used in place of HOBt.
^{b}LCMS data reported as (M/2 + H)⁺.

### Example 5: Synthesis of Compounds of Formula 1 (Method 5)

### Synthesis of 7-(4-(R)-3-(4-(N-(4-(4-(4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazine-1-carbonyl)-N—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-2-azaspiro[3.5]nonane-2-carboxamide (I-210)

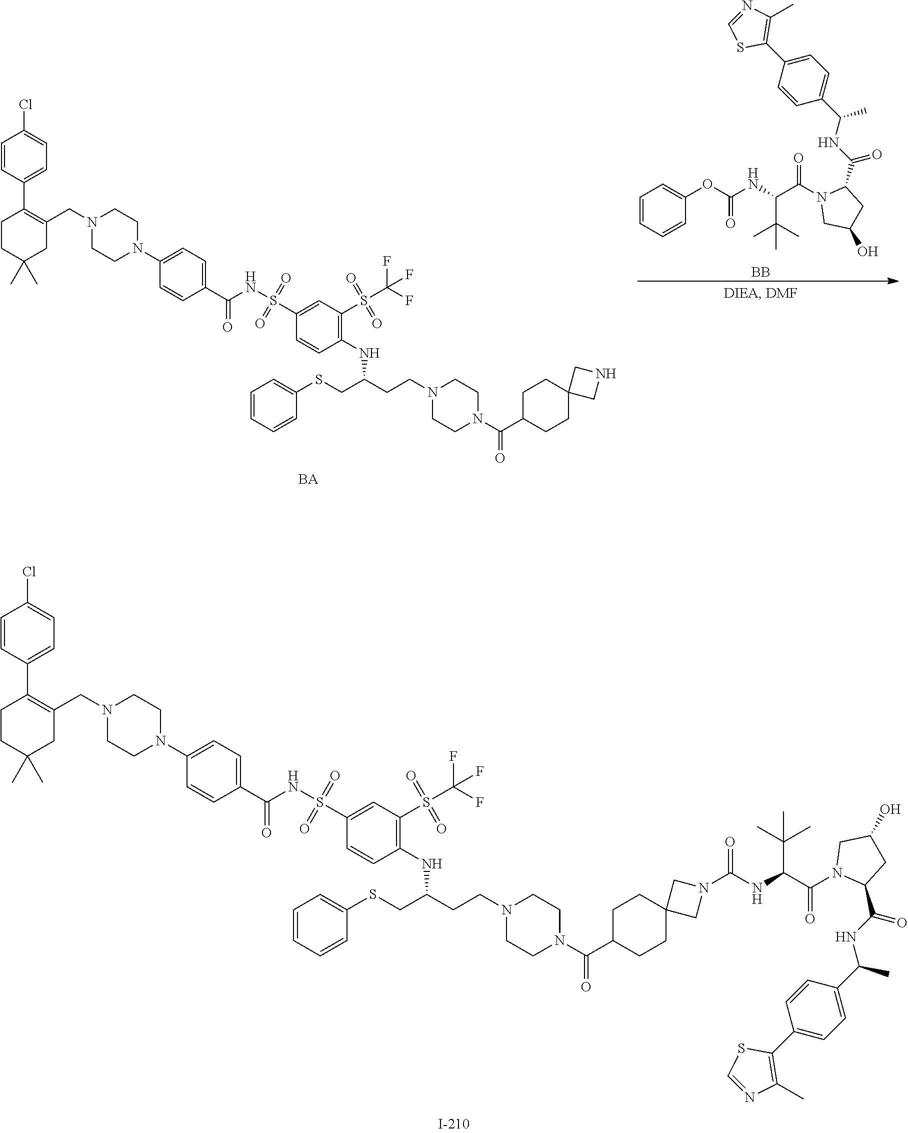 [SMILES: CC1(C)CCC(c2ccc(Cl)cc2)=C(CN2CCN(c3ccc(C(=O)NS(=O)(=O)c4ccc(N[C@H](CCN5CCN(C(=O)C6CCC7(CC6)CNC7)CC5)CSc5ccccc5)c(S(=O)(=O)C(F)(F)F)c4)cc3)CC2)C1 ; Cc1ncsc1-c1ccc([C@H](C)NC(=O)[C@@H]2C[C@@H](O)CN2C(=O)[C@@H](NC(=O)Oc2ccccc2)C(C)(C)C)cc1 ; "BB" ; "BA" ; Cc1ncsc1-c1ccc([C@H](C)NC(=O)[C@@H]2C[C@@H](O)CN2C(=O)[C@@H](NC(=O)N2CC3(CCC(C(=O)N4CCN(CC[C@H](CSc5ccccc5)Nc5ccc(S(=O)(=O)NC(=O)c6ccc(N7CCN(CC8=C(c9ccc(Cl)cc9)CCC(C)(C)C8)CC7)cc6)cc5S(=O)(=O)C(F)(F)F)CC4)CC3)C2)C(C)(C)C)cc1]

To solution of (R)—N-((4-((4-(4-(2-azaspiro[3.5]nonane-7-carbonyl)piperazin-1-yl)-1-(phenylthio)butan-2-yl)amino)-3-((trifluoromethyl)sulfonyl)phenyl)sulfonyl)-4-(4-((4′-chloro-4,4-dimethyl-3,4,5,6-tetrahydro-[1,1′-biphenyl]-2-yl)methyl)piperazin-1-yl)benzamide (55 mg, 49 μmol, Intermediate BA) and phenyl N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamate (27.6 mg, 48.9 μmol, Intermediate BB) in DMF (1 mL) was added DIEA (31.6 mg, 245 μmol), then the mixture was stirred at 80° C. for 2 h. On completion, the mixture was purified by prep-HPLC (neutral condition: column: Phenomenex C18 150*25 mm*10 um; mobile phase: [water (NH₄HCO₃)-ACN]; B %: 53%-63%, 8 min) to give the title compound (19 mg, 24% yield) as a white solid. LC-MS (ESI⁺) m/z 1594.9 (M+H)⁺. ¹H NMR (400 MHz, DMSO-d₆) δ=8.98 (s, 1H), 8.40-8.33 (m, 1H), 8.12 (s, 1H), 7.98-7.92 (m, 1H), 7.71 (br d, J=8.4 Hz, 2H), 7.45 (br d, J=2.0 Hz, 2H), 7.39-7.35 (m, 4H), 7.34-7.31 (m, 2H), 7.26 (br t, J=7.2 Hz, 2H), 7.20-7.15 (m, 1H), 7.14-7.10 (m, 2H), 6.88-6.82 (m, 2H), 5.55 (br d, J=8.8 Hz, 1H), 5.12-5.06 (m, 1H), 4.94-4.87 (m, 1H), 4.45-4.39 (m, 1H), 4.33-4.26 (m, 2H), 4.11-4.04 (m, 1H), 3.63-3.41 (m, 9H), 3.24-3.15 (m, 5H), 2.89 (s, 1H), 2.73 (s, 1H), 2.69-2.65 (m, 3H), 2.45 (br s, 3H), 2.33 (br s, 4H), 2.27-2.21 (m, 5H), 1.99 (br s, 4H), 1.84-1.74 (m, 4H), 1.60-1.39 (m, 8H), 1.37 (br d, J=7.2 Hz, 3H), 1.34-1.22 (m, 3H), 0.95 (br d, J=17.6 Hz, 14H).

TABLE 6
Compounds synthesized via Method 5, using the corresponding
amines and carbamates for the coupling.

| | | | LCMS | |
|---|---|---|---|---|
| | | | (ESI+) | |
| | | | m/z | |
| I-# | Amine | Carbamate | (M + H)⁺ | ¹H NMR (400 MHz, DMSO-d6) |
| I-208 | BK | BL | 1625.0 | 8.98 (s, 1H), 8.37 (d, J = 7.2 Hz, |
| | | | | 1H), 8.14-8.10 (m, 1H), 7.95 (br |
| | | | | d, J = 9.6 Hz, 1H), 7.71 (br d, J = |
| | | | | 8.4 Hz, 2H), 7.45-7.42 (m, 2H), |
| | | | | 7.37 (d, J = 8.4 Hz, 4H), 7.35- |
| | | | | 7.32 (m, 2H), 7.27 (t, J = 7.6 Hz, |
| | | | | 2H), 7.20-7.15 (m, 1H), 7.12 (d, |
| | | | | J = 8.4 Hz, 2H), 7.03-6.94 (m, |
| | | | | 1H), 6.85 (br d, J = 8.0 Hz, 2H), |
| | | | | 5.69-5.63 (m, 1H), 5.11-5.05 |
| | | | | (m, 1H), 4.94-4.87 (m, 1H), 4.45- |
| | | | | 4.36 (m, 2H), 4.28 (br s, 1H), |
| | | | | 4.11-4.03 (m, 1H), 3.65-3.58 |
| | | | | (m, 2H), 2.85-2.71 (m, 2H), 2.52 |
| | | | | (br d, J = 2.0 Hz, 9H), 2.45 (s, |
| | | | | 3H), 2.31-2.13 (m, 9H), 1.99 (br |
| | | | | s, 6H), 1.79 (ddd, J = 4.4, 8.4, |
| | | | | 12.8 Hz, 2H), 1.68-1.62 (m, 2H), |
| | | | | 1.53-1.31 (m, 14H), 1.24 (br s, |
| | | | | 3H), 1.18-1.08 (m, 3H), 1.06 (t, |
| | | | | J = 6.8 Hz, 1H), 0.95 (br d, J = |
| | | | | 13.2 Hz, 16H) |
| I-209 | BK | BM | 1551.6 | 8.36 (d, J = 7.6 Hz, 1H), 8.07 (d, |
| | | | | J = 1.2 Hz, 1H), 7.92 (dd, J = 2.0, |
| | | | | 8.8 Hz, 1H), 7.70 (d, J = 8.8 Hz, |
| | | | | 2H), 7.42-7.34 (m, 6H), 7.31- |
| | | | | 7.27 (m, 4H), 7.22-7.17 (m, 1H), |
| | | | | 7.12 (d, J = 8.4 Hz, 2H), 6.79 (br |
| | | | | d, J = 8.8 Hz, 2H), 5.65 (br d, J = |
| | | | | 8.8 Hz, 1H), 5.07 (d, J = 3.6 Hz, |
| | | | | 1H), 4.86 (br t, J = 7.2 Hz, 1H), |
| | | | | 4.44-4.34 (m, 2H), 4.26 (br s, |
| | | | | 1H), 4.11 (s, 1H), 4.06-3.98 (m, |
| | | | | 1H), 3.65-3.56 (m, 3H), 3.43- |
| | | | | 3.37 (m, 4H), 3.14 (br s, 4H), 2.75 |
| | | | | (br d, J = 1.2 Hz, 3H), 2.67 (br d, |
| | | | | J = 1.6 Hz, 1H), 2.35-2.19 (m, |
| | | | | 12H), 2.02-1.94 (m, 4H), 1.78- |
| | | | | 1.69 (m, 2H), 1.69-1.60 (m, 3H), |
| | | | | 1.51-1.37 (m, 9H), 1.32 (d, J = |
| | | | | 7.2 Hz, 3H), 1.28-1.20 (m, 6H), |
| | | | | 1.17-1.08 (m, 3H), 0.95 (br d, |
| | | | | J = 14.8 Hz, 15H) |
| I-210 | BA | BB | 1594.9 | 8.98 (s, 1H), 8.40-8.33 (m, 1H), |
| | | | | 8.12 (s, 1H), 7.98-7.92 (m, 1H), |
| | | | | 7.71 (br d, J = 8.4 Hz, 2H), 7.45 |
| | | | | (br d, J = 2.0 Hz, 2H), 7.39-7.35 |
| | | | | (m, 4H), 7.34-7.31 (m, 2H), 7.26 |
| | | | | (br t, J = 7.2 Hz, 2H), 7.20-7.15 |
| | | | | (m, 1H), 7.14-7.10 (m, 2H), 6.88- |
| | | | | 6.82 (m, 2H), 5.55 (br d, J = 8.8 |
| | | | | Hz, 1H), 5.12-5.06 (m, 1H), 4.94- |
| | | | | 4.87 (m, 1H), 4.45-4.39 (m, |
| | | | | 1H), 4.33-4.26 (m, 2H), 4.11- |
| | | | | 4.04 (m, 1H), 3.63-3.41 (m, 9H), |
| | | | | 3.24-3.15 (m, 5H), 2.89 (s, 1H), |
| | | | | 2.73 (s, 1H), 2.69-2.65 (m, 3H), |
| | | | | 2.45 (br s, 3H), 2.33 (br s, 4H), |
| | | | | 2.27-2.21 (m, 5H), 1.99 (br s, |
| | | | | 4H), 1.84-1.74 (m, 4H), 1.60- |
| | | | | 1.39 (m, 8H), 1.37 (br d, J = 7.2 |
| | | | | Hz, 3H), 1.34-1.22 (m, 3H), 0.95 |
| | | | | (br d, J = 17.6 Hz, 14H) |

### Example 6: Synthesis of Compounds of Formula I (Method 7)

### Synthesis of (2S,4R)—N-(2-(2-(1-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperidin-4-yl)ethoxy)-4 (4-methylthiazol-5-yl)benzyl)-1-((S)-2-(1-fluorocyclopropanecarboxamido)-3,3-dimethylbutanoyl)-4-hydroxypyrrolidine-2-carboxamide (I-234)

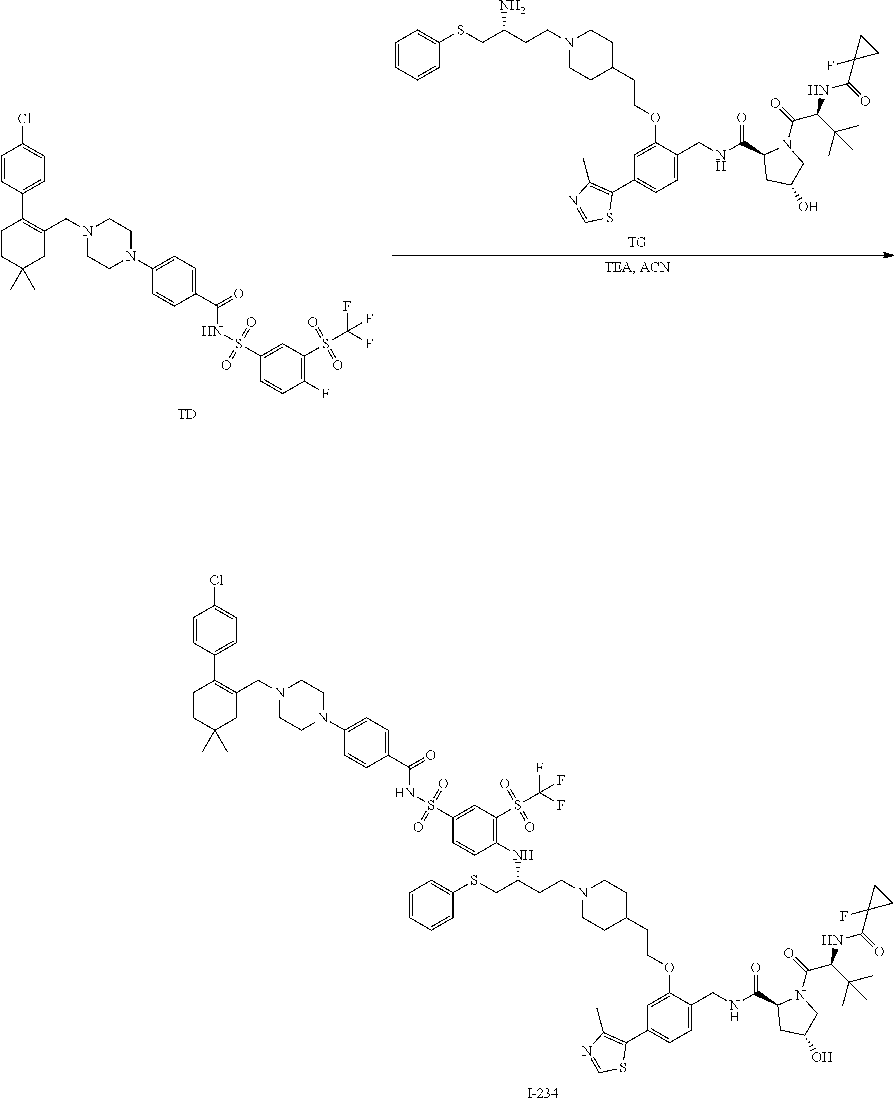 [SMILES: CC1(C)CCC(c2ccc(Cl)cc2)=C(CN2CCN(c3ccc(C(=O)NS(=O)(=O)c4ccc(F)c(S(=O)(=O)C(F)(F)F)c4)cc3)CC2)C1 ; Cc1ncsc1-c1ccc(CNC(=O)[C@@H]2C[C@@H](O)CN2C(=O)[C@@H](NC(=O)C2(F)CC2)C(C)(C)C)c(OCCC2CCN(CC[C@@H](N)CSc3ccccc3)CC2)c1 ; "TD" ; Cc1ncsc1-c1ccc(CNC(=O)[C@@H]2C[C@@H](O)CN2C(=O)[C@@H](NC(=O)C2(F)CC2)C(C)(C)C)c(OCCC2CCN(CC[C@H](CSc3ccccc3)Nc3ccc(S(=O)(=O)NC(=O)c4ccc(N5CCN(CC6=C(c7ccc(Cl)cc7)CCC(C)(C)C6)CC5)cc4)cc3S(=O)(=O)C(F)(F)F)CC2)c1]

A mixture of (2S,4R)—N-(2-(2-(1-((R)-3-amino-4-(phenylthio)butyl)piperidin-4-yl)ethoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (44 mg, 51.2 μmol, HCl, Intermediate TG), 4-(4-((4′-chloro-4,4-dimethyl-3,4,5,6-tetrahydro-[1,1′-biphenyl]-2-yl)methyl)piperazin-1-yl)-N-((4-fluoro-3-((trifluoromethyl)sulfonyl)phenyl)sulfonyl)benzamide (37.3 mg, 51.2 μmol, Intermediate TD), and TEA (41.4 mg, 410 μmol, 57.0 uL) in ACN (0.3 mL) was stirred at 60° C. for 12 h. On completion, the filtered was filtrated and concentrated to give a residue. The residue was purified by reversed-phase HPLC (column: Phenomenex luna C18 250*50 mm*15 um; mobile phase: [water (FA)-ACN]; B %: 33%-63%, 15 min) to give the title compound (29.03 mg, 37% yield) as a white solid. LC-MS (ESI⁺) m/z 1529.6 (M+H)⁺; ¹H NMR (400 MHz, DMSO-d₆) δ 8.98 (s, 1H), 8.50 (br t, J=5.9 Hz, 1H), 8.07 (s, 1H), 7.95 (br d, J=9.4 Hz, 1H), 7.70 (br d, J=8.8 Hz, 2H), 7.42-7.33 (m, 6H), 7.29 (br t, J=7.4 Hz, 4H), 7.21-7.17 (m, 1H), 7.12 (br d, J=8.4 Hz, 2H), 7.00 (s, 1H), 6.95 (brd, J=8.5 Hz, 1H), 6.90-6.85 (m, 1H), 6.77 (br d, J=8.4 Hz, 2H), 6.71-6.65 (m, 1H), 5.18 (d, J=3.2 Hz, 1H), 4.59 (br d, J=9.2 Hz, 1H), 4.54-4.49 (m, 1H), 4.37-4.33 (m, 1H), 4.32-4.26 (m, 1H), 4.20 (br d, J=6.0 Hz, 1H), 4.19-4.15 (m, 1H), 4.07 (br s, 2H), 4.02-3.96 (m, 1H), 3.69-3.55 (m, 3H), 3.12 (br s, 5H), 2.73 (br s, 3H), 2.67 (br s, 1H), 2.54 (br s, 1H), 2.45 (s, 3H), 2.33 (br s, 1H), 2.24 (br d, J=15.6 Hz, 6H), 2.13-2.05 (m, 2H), 1.98 (br s, 3H), 1.95-1.86 (m, 3H), 1.70 (br s, 3H), 1.46-1.39 (m, 3H), 1.37 (br s, 1H), 1.34 (br dd, J=4.0, 5.2 Hz, 2H), 1.22 (br d, J=8.4 Hz, 3H), 0.95 (br d, J=3.6 Hz, 12H).

TABLE 7
Compounds synthesized via Method 5, using the corresponding
amines and fluorines for the coupling.

| | | | LCMS | |
|---|---|---|---|---|
| | | | (ESI+) | |
| | | | m/z | |
| I-# | Amine | Flourine | (M + H)⁺ | ¹H NMR (400 MHz, DMSO-d6) |
| I-235 | TO | TD | 1503.0 | 8.98 (s, 1H), 8.51 (t, J = 6.0 Hz, 1H), |
| | | | | 8.07 (d, J = 2.0 Hz, 1H), 7.96 (d, J = |
| | | | | 7.6 Hz, 1H), 7.70 (d, J = 8.8 Hz, 2H), |
| | | | | 7.44-7.33 (m, 6H), 7.29 (t, J = 7.6 |
| | | | | Hz, 3H), 7.24-7.16 (m, 1H), 7.12 (d, |
| | | | | J = 8.4 Hz, 2H), 7.00-6.92 (m, 2H), |
| | | | | 6.88-6.81 (m, 1H), 6.76 (d, J = 9.2 |
| | | | | Hz, 3H), 5.24-5.11 (m, 1H), 4.59 (d, |
| | | | | J = 9.2 Hz, 1H), 4.53 (t, J = 8.0 Hz, |
| | | | | 1H), 4.39-4.22 (m, 3H), 4.18-4.12 |
| | | | | (m, 1H), 4.07-3.84 (m, 6H), 3.65- |
| | | | | 3.60 (m, 2H), 3.11 (s, 4H), 2.77- |
| | | | | 2.71 (m, 3H), 2.44 (s, 3H), 2.32- |
| | | | | 2.16 (m, 8H), 2.10-1.97 (m, 6H), |
| | | | | 1.94-1.89 (m, 1H), 1.84-1.73 (m, |
| | | | | 2H), 1.46-1.39 (m, 3H), 1.38-1.30 |
| | | | | (m, 2H), 1.22 (dd, J = 2.8, 8.4 Hz, |
| | | | | 2H), 0.95 (d, J = 6.0 Hz, 15H) |
| I-236 | TJ | TD | 1492.9 | 8.98 (s, 1H), 8.53 (br t, J = 5.2 Hz, |
| | | | | 1H), 8.09 (s, 1H), 7.96 (br d, J = 8.8 |
| | | | | Hz, 1H), 7.74-7.66 (m, 2H), 7.42- |
| | | | | 7.22 (m, 10H), 7.13 (s, 3H), 6.98- |
| | | | | 6.93 (m, 2H), 6.78 (br d, J = 8.8 Hz, |
| | | | | 2H), 5.19 (br d, J = 3.2 Hz, 1H), 4.59 |
| | | | | (br d, J = 9.6 Hz, 1H), 4.52 (br t, J = |
| | | | | 8.4 Hz, 1H), 4.38-4.25 (m, 2H), 4.21 |
| | | | | (br d, J = 5.2 Hz, 1H), 4.11-3.99 (m, |
| | | | | 3H), 3.94 (d, J = 7.2 Hz, 1H), 3.69- |
| | | | | 3.57 (m, 2H), 3.13 (br d, J = 1.2 Hz, |
| | | | | 5H), 2.81-2.70 (m, 3H), 2.67 (br s, |
| | | | | 1H), 2.44 (s, 3H), 2.36-2.18 (m, |
| | | | | 7H), 2.07 (br d, J = 6.0 Hz, 4H), 1.99 |
| | | | | (br s, 3H), 1.91 (br dd, J = 4., 9.2 Hz, |
| | | | | 2H), 1.69-1.54 (m, 1H), 1.48-1.30 |
| | | | | (m, 5H), 1.27-1.16 (m, 3H), 1.16- |
| | | | | 1.08 (m, 1H), 0.95 (br d, J = 7.2 Hz, |
| | | | | 15H) |
| I-242 | UE | TD | 1502.2 | 10.29-9.87 (m, 1H), 8.99 (s, 1H), |
| | | | | 8.54-8.46 (m, 1H), 8.18 (d, J = 2.0 |
| | | | | Hz, 1H), 8.04-7.97 (m, 1H), 7.77 (br |
| | | | | d, J = 8.4 Hz, 2H), 7.46-7.39 (m, |
| | | | | 3H), 7.32-7.27 (m, 3H), 7.26-7.20 |
| | | | | (m, 3H), 7.17-7.11 (m, 3H), 7.06 (s, |
| | | | | 1H), 6.96 (br t, J = 8.0 Hz, 3H), 5.36- |
| | | | | 5.15 (m, 1H), 4.89 (br s, 1H), 4.64- |
| | | | | 4.46 (m, 2H), 4.38-4.15 (m, 4H), |
| | | | | 3.92-3.85 (m, 1H), 2.91 (br dd, J = |
| | | | | 7.2, 12.5 Hz, 2H), 2.84-2.71 (m, |
| | | | | 3H), 2.45 (s, 3H), 2.34-2.31 (m, |
| | | | | 1H), 2.27 (br s, 3H), 2.21-2.14 (m, |
| | | | | 3H), 2.10 (br s, 4H), 2.07 (s, 2H), |
| | | | | 2.00 (br dd, J = 6.8, 14.8 Hz, 2H), |
| | | | | 1.94-1.85 (m, 2H), 1.51-1.44 (m, |
| | | | | 3H), 1.40-1.29 (m, 3H), 1.26-1.19 |
| | | | | (m, 6H), 1.16 (t, J = 7.2 Hz, 2H), 1.05 |
| | | | | (t, J = 7.2 Hz, 2H), 1.00 (s, 6H), 0.96- |
| | | | | 0.90 (m, 8H), 0.85 (br t, J = 7.2 Hz, |
| | | | | 1H) |
| I-243 | UG | TD | 1518.0 | 12.17 (br s, 1H), 10.38-9.89 (m, |
| | | | | 2H), 8.99 (s, 1H), 8.57 (br t, J = 5.6 |
| | | | | Hz, 1H), 8.18 (d, J = 2.0 Hz, 1H), |
| | | | | 8.00 (dd, J = 2.0, 9.2 Hz, 1H), 7.78 |
| | | | | (br d, J = 8.8 Hz, 2H), 7.44-7.38 (m, |
| | | | | 3H), 7.33-7.26 (m, 3H), 7.25-7.20 |
| | | | | (m, 3H), 7.17 (br d, J = 8.4 Hz, 2H), |
| | | | | 7.00-6.94 (m, 4H), 4.60 (d, J = 9.2 |
| | | | | Hz, 1H), 4.53 (t, J = 8.0 Hz, 1H), 4.35 |
| | | | | (br s, 1H), 4.30 (br d, J = 5.6 Hz, 1H), |
| | | | | 4.21 (br dd, J = 4.8, 16.0 Hz, 2H), |
| | | | | 3.98-3.84 (m, 4H), 3.68-3.56 (m, |
| | | | | 8H), 3.40-3.32 (m, 6H), 3.17-3.11 |
| | | | | (m, 1H), 3.06-2.98 (m, 1H), 2.95- |
| | | | | 2.88 (m, 1H), 2.83-2.72 (m, 2H), |
| | | | | 2.45 (s, 3H), 2.28 (br s, 2H), 2.21- |
| | | | | 2.12 (m, 4H), 2.11-2.07 (m, 1H), |
| | | | | 2.01-1.88 (m, 3H), 1.79-1.66 (m, |
| | | | | 2H), 1.47 (br t, J = 6.0 Hz, 2H), 1.41- |
| | | | | 1.36 (m, 1H), 1.36-1.31 (m, 1H), |
| | | | | 1.22 (br dd, J = 2.8, 8.4 Hz, 2H), 1.00 |
| | | | | (s, 6H), 0.95 (s, 9H |

### Example 7: Synthesis of Compounds of Formula I (Method 8)

### Synthesis of (2S,4R)—N-(2-(3-(4-((R)-3-((4-(N-(4-(4-((4′-chloro-4,4-dimethyl-3,4,5,6-tetrahydro-[1,1′-biphenyl]-2-yl)methyl)piperazin-1-yl)benzoyl)sulfamoyl)-2-((trifluoromethyl)sulfonyl)phenyl)amino)-4-(phenylthio)butyl)piperazin-1-yl)propoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (I-230)

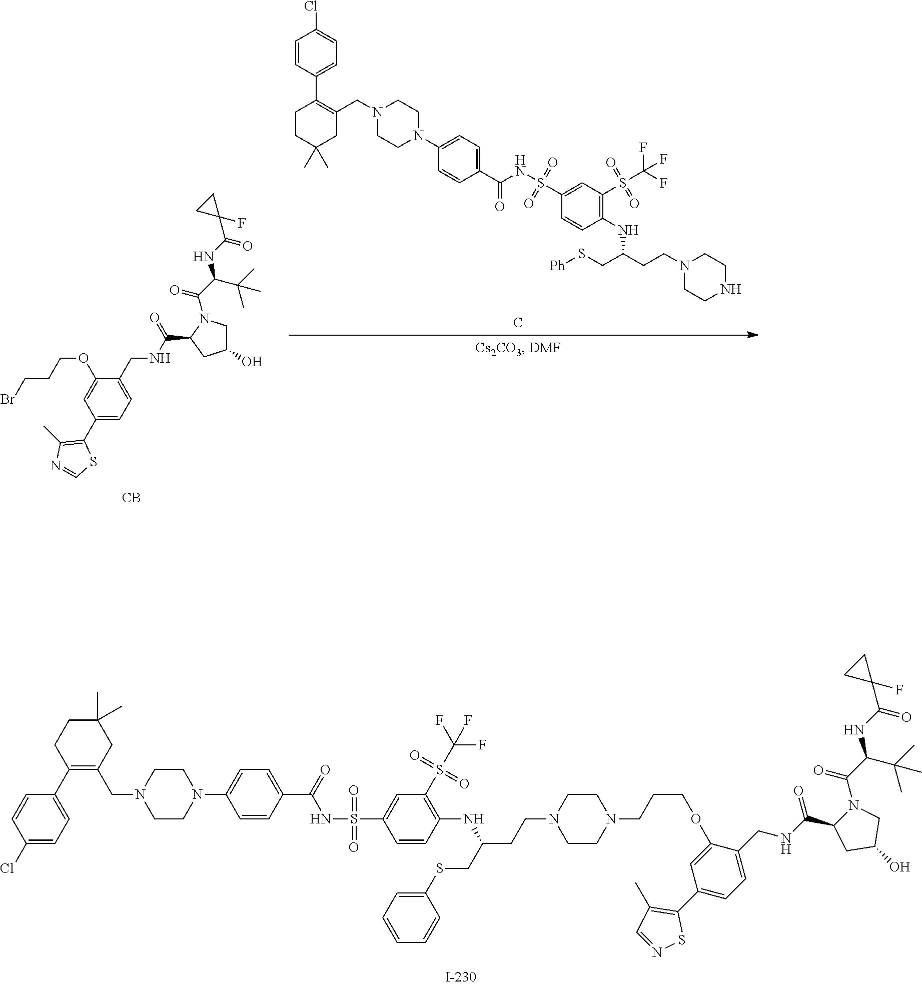

To a solution of (2S,4R)—N-(2-(3-bromopropoxy)-4-(4-methylthiazol-5-yl)benzyl)-1-((S)-2-(1-fluorocyclopropanecarboxamido)-3,3-dimethylbutanoyl)-4-hydroxypyrrolidine-2-carboxamide (80 mg, 61.2 μmol, Intermediate CB) and (R)-4-(4-((4′-chloro-4,4-dimethyl-3,4,5,6-tetrahydro-[1,1′-biphenyl]-2-yl)methyl)piperazin-1-yl)-N-((4-((1-(phenylthio)-4-(piperazin-1-yl)butan-2-yl)amino)-3-((trifluoromethyl)sulfonyl)phenyl)sulfonyl)benzamide (59.6 mg, 61.2 μmol, Intermediate C) in DMF (1 mL) was added Cs₂CO₃ (39.9 mg, 122 μmol). The mixture was stirred at 90° C. for 12 h. On completion, the reaction mixture was filtered and concentrated under reduced pressure to give a residue. The residue was purified by prep-HPLC (column: Phenomenex luna C18 150*25 mm*10 um; mobile phase: [water (FA)-ACN]; B %: 33%-63%, 10 min) to give the title compound (28 mg, 28% yield, 95% purity) as a yellow solid. ¹H NMR (400 MHz, DMSO-d₆) δ 8.99-8.96 (m, 1H), 8.53-8.47 (m, 1H), 8.07 (s, 1H), 7.95 (br d, J=8.0 Hz, 1H), 7.70 (br d, J=8.6 Hz, 2H), 7.42 (br d, J=7.6 Hz, 1H), 7.36 (br t, J=8.4 Hz, 4H), 7.32-7.23 (m, 4H), 7.19 (br t, J=7.2 Hz, 1H), 7.11 (d, J=8.4 Hz, 2H), 7.00-6.95 (m, 2H), 6.92-6.85 (m, 1H), 6.81-6.68 (m, 3H), 5.17 (br d, J=3.4 Hz, 1H), 4.63-4.56 (m, 1H), 4.52 (br t, J=8.0 Hz, 1H), 4.38-4.17 (m, 4H), 4.13-3.97 (m, 4H), 3.67-3.57 (m, 2H), 3.17-3.08 (m, 5H), 2.74 (br s, 2H), 2.46-2.44 (m, 8H), 2.29-2.19 (m, 8H), 2.13-2.04 (m, 3H), 2.02-1.88 (m, 7H), 1.45-1.30 (m, 5H), 1.22 (br dd, J=2.8, 8.6 Hz, 2H), 0.95 (br d, J=3.2 Hz, 15H); LC-MS (ESI⁺) m/z 1548.1 (M+H)⁺.

### Example 8. BCL-XL Degradation Assay

Compounds treatment: compounds were reconstituted in DMSO to make a stock concentration of 10 mM. MOLT-4 cells were maintained in RPMI-1640+10% FBS+1% Penicillin-Streptomycin. Cells were seeded into 6-well plates in 2 mL at the seeding density of 2e6 cells/mL. Compounds were added to the cells to a final concentration ranging 0.02-10 μM. After 16 hr incubation at 37° C. in a CO₂ incubator, cells were collected in an Eppendorf tube and centrifuged at 200×g for 5 minutes, the supernatant removed and pellets washed with ice-cold PBS, centrifuged and supernatant removed again. Then, 50 μl of pre-chilled RIPA lysis buffer (Boston BioProducts, BP-115D) with protease and phosphatase inhibitors (Roche Applied Science, #04906837001 and #04693116001) was added into cell pellet to lyze the cells for 20 minutes under 4° C. Cell lysates were spin at 13000 rpm for 10 minutes. Supernatants were collected and protein concentration was measured by BCA assay kit (Solarbio, PC0020-500) following the manufacturer's protocol. Samples final protein concentration was adjusted to 3.75 ug/uL with the RIPA lysis buffer based on BCA assay results and 4× loading buffer (Invitrogen, NP0007) was added into the samples and samples were heated to 85° C. for 10 min and then let cool at RT. Samples were centrifuged at 13000 rpm for 10 min and then loaded into a 26-well NuPAGER: Novex 4-12% Bis-Tris Midi Gel (Invitrogen, WG1403BOX) and run in NuPAGE® MOPS SDS Running Buffer (Invitrogen, NP0001) for 180 minutes at 70 V into a XCell4 SureLock™ Midi-Cell Runner (Invitrogen, WR0100). Gel were transferred using the Invitrogen iBlot gel transfer device (Invitrogen. IB21001) and the nitrocellulose transfer stacks (Invitrogen, IB23001) with Program PO for 9 minutes. The membrane was then blocked with 5% bovin serum albumin for 1 hr at RT, then incubated with primary antibodies diluted in blocking buffer (LI-COR, 927-60001) and shake at 4° C. overnight. The primary antibodies were anti-BCL-xL antibody (CST, 2762:1:1000), anti-beta-actin (CST, 3700:1:5000) and anti-GAPDH (CST, 97166:1:5000). Membranes were then washed three times with PBST for 15 minutes at RT. Secondary antibodies IR-Dye 680 CW Goat anti Mouse IgG (LI-COR, 926-68070) and IR-Dye 800 CW Goat anti Rabbit IgG (LI-COR, 926-32211) were diluted at a ratio of 1:10000 into PBST and membranes incubated for 1 hour at RT. Membranes were then washed three times with PBST for 15 minutes at RT. The western blot images were obtained using Odyssey Imaging System and quantified using Image Studio Lite Ver 5.2 software.

BCL-XL degradation results in MOLT-4 cells for compounds of the invention are presented in Table 9A and 9B. The letter codes for BCL-XL DC₅₀ include: A (<0.01 μM); B (0.01-0.1 μM); C (>0.1-0.5 μM); D (>0.5-1.0 μM); and E (>1.0 μM). The letter codes for Dmax % include A (>75%); B (50 to 75%); C (<50%). “-” is inactive or not tested.

TABLE 8
BCL-XL Degradation Results

| | Bcl-xL | |
|---|---|---|
| | ELISA in | Bcl-xL |
| | MOLT-4 | ELISA in |
| | degradation | MOLT-4 |
| | 6 h: External | degradation |
| I-# | Abs-DC₅₀ | 6 h: Dmax % |
| I-4 | D | B |
| I-5 | B | A |
| I-6 | D | C |
| I-7 | D | C |
| I-8 | C | B |
| I-9 | D | C |
| I-10 | C | B |
| I-11 | D | C |
| I-12 | D | C |
| I-13 | D | B |
| I-14 | D | C |
| I-21 | D | C |
| I-22 | B | A |
| I-23 | D | C |
| I-24 | D | C |
| I-25 | B | A |
| I-27 | D | C |
| I-28 | B | A |
| I-29 | B | A |
| I-31 | B | B |
| I-33 | C | B |
| I-34 | D | B |
| I-35 | B | A |
| I-36 | D | C |
| I-37 | D | C |
| I-38 | C | B |
| I-39 | C | B |
| I-40 | D | B |
| I-41 | D | C |
| I-42 | B | A |
| I-43 | D | C |
| I-51 | C | B |
| I-52 | C | B |
| I-60 | D | C |
| I-63 | C | B |
| I-65 | B | B |
| I-67 | D | B |
| I-69 | D | C |
| I-74 | D | C |
| I-81 | B | A |
| I-85 | D | C |
| I-86 | B | B |
| I-98 | B | B |
| I-102 | A | A |
| I-105 | C | B |
| I-106 | B | B |
| I-108 | B | B |
| I-109 | B | A |
| I-113 | A | A |
| I-118 | B | A |
| I-120 | B | B |
| I-121 | A | A |
| I-122 | B | A |
| I-124 | A | B |
| I-125 | B | B |
| I-126 | B | B |
| I-127 | D | B |
| I-131 | D | C |
| I-133 | D | C |
| I-134 | D | C |
| I-135 | B | A |

### Example 9. CTG Cell Viability Assay

Compound-mediated viability effect on MOLT-4 and Caki-2 was quantitatively determined using the CellTiter-Glo® Luminescent Cell Viability Assay kit from Promega (Catalog number G7570) following manufacturer's recommended procedures. Briefly, MOLT-4 and Caki-2 cells were seeded into 384 well plates (Grenier Bio-One, Catalog number 781080) with a density of 10,000 cells per well. Compounds were then added to the assay plate with final top concentration of 10 UM and 1:3 dilution series with total of 9 doses. The final DMSO concentration is normalized to 0.2%. The assay plates were incubated at 37° C. for 3 days under 5% CO₂. Then the assay plate was equilibrated at room temperature for 10 minutes. To determine cell viability, 30 uL CellTiter Glo reagent was added to each well and the assay plate was centrifuged at 1000 rpm for 30 second, incubated at room temperature for 10 min, and analyzed by detecting the luminescence using a multimode plate reader (EnVision 2105, PerkinElmer). The data was then analyzed by software Prism 7.0 from GraphPad and the dose response curves are fit using a three-parameter logistic equation to calculate IC₅₀.

MOLT-4 and Caki-2 cell viability results for compounds of the invention are presented in Table 10. The letter codes for MOLT-4 IC₅₀ include: A (<0.1 μM); B (0.1-1.0 μM); C (>1.0-3.0 μM); and D (>3.0 μM). The letter codes for Caki-2 IC₅₀ include: A (<5 μM); B (5-10 μM); C (>10-30.0 μM); and D (>30.0 μM). “-” is inactive or not tested.

TABLE 9
Cell Viability Results

| | CTG Cell | CTG |
|---|---|---|
| | Viability | Chempartner/ |
| | Assay - MOLT-4 | PHA- Caki-2 |
| | 3 day: | 3-day: |
| | Average | Average |
| I-# | external-IC₅₀ | external-IC₅₀ |
| I-4 | D | D |
| I-5 | B | D |
| I-6 | D | D |
| I-7 | D | D |
| I-8 | B | D |
| I-9 | D | D |
| I-10 | D | D |
| I-11 | D | D |
| I-12 | D | D |
| I-13 | D | D |
| I-14 | D | D |
| I-15 | D | D |
| I-16 | D | D |
| I-17 | D | D |
| I-18 | D | D |
| I-20 | D | D |
| I-21 | D | D |
| I-22 | B | C |
| I-23 | B | D |
| I-24 | D | D |
| I-25 | B | D |
| I-27 | D | D |
| I-28 | A | C |
| I-29 | B | C |
| I-30 | D | D |
| I-31 | B | A |
| I-33 | C | D |
| I-34 | D | D |
| I-35 | D | D |
| I-36 | D | D |
| I-37 | D | D |
| I-38 | B | D |
| I-39 | D | D |
| I-40 | D | D |
| I-41 | D | D |
| I-42 | A | B |
| I-43 | D | D |
| I-44 | D | D |
| I-45 | D | D |
| I-46 | D | D |
| I-47 | D | D |
| I-48 | D | D |
| I-49 | D | D |
| I-50 | D | D |
| I-51 | C | D |
| I-52 | B | D |
| I-53 | D | D |
| I-54 | D | D |
| I-55 | D | D |
| I-56 | D | D |
| I-57 | D | D |
| I-58 | C | D |
| I-59 | D | D |
| I-60 | C | D |
| I-61 | D | D |
| I-62 | D | D |
| I-63 | C | D |
| I-64 | D | D |
| I-65 | A | C |
| I-66 | D | D |
| I-67 | D | D |
| I-68 | D | D |
| I-69 | D | D |
| I-70 | D | D |
| I-71 | C | C |
| I-72 | C | C |
| I-73 | D | D |
| I-74 | B | D |
| I-75 | B | D |
| I-76 | D | D |
| I-77 | B | C |
| I-78 | D | D |
| I-79 | D | D |
| I-80 | D | D |
| I-81 | A | D |
| I-82 | C | C |
| I-83 | D | D |
| I-84 | D | D |
| I-85 | B | D |
| I-86 | B | D |
| I-87 | D | D |
| I-88 | C | D |
| I-89 | D | D |
| I-90 | D | D |
| I-91 | D | D |
| I-92 | D | D |
| I-93 | C | D |
| I-94 | D | D |
| I-95 | D | D |
| I-96 | B | D |
| I-97 | D | D |
| I-98 | B | D |
| I-99 | D | D |
| I-100 | D | D |
| I-101 | D | D |
| I-102 | A | D |
| I-103 | B | D |
| I-104 | D | D |
| I-105 | B | D |
| I-106 | A | D |
| I-107 | B | D |
| I-108 | A | D |
| I-109 | B | D |
| I-110 | B | D |
| I-111 | D | D |
| I-112 | D | D |
| I-113 | B | D |
| I-114 | D | D |
| I-115 | D | D |
| I-116 | D | D |
| I-117 | D | D |
| I-118 | B | D |
| I-119 | D | D |
| I-120 | A | C |
| I-121 | A | C |
| I-122 | B | D |
| I-123 | D | D |
| I-124 | A | C |
| I-125 | B | D |
| I-126 | A | A |
| I-127 | D | D |
| I-128 | D | D |
| I-129 | D | D |
| I-130 | C | D |
| I-131 | B | D |
| I-132 | B | D |
| I-133 | B | D |
| I-134 | B | A |
| I-135 | A | C |
| I-136 | B | D |
| I-137 | A | D |
| I-138 | B | C |
| I-139 | A | D |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I-aa-1: 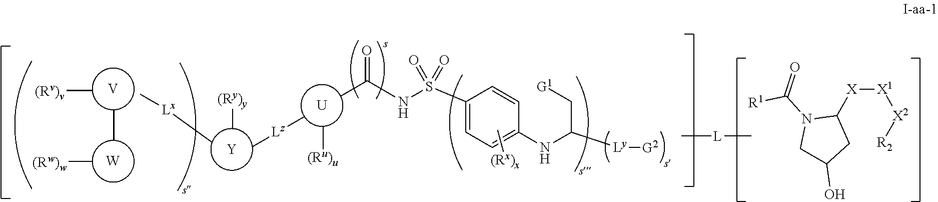
or a pharmaceutically acceptable salt, wherein:
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Ring W is a ring selected from phenyl, naphthyl, a 4-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 5-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
G¹ is —S-aryl, —S-heteroaryl, or —R^{A},
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
G² is hydrogen, halogen, —CN, —OR, —SR, —N(R)₂, —S(O)₂R, —S(O)₂N(R)₂, —C(O)R, —C(O)OR, or 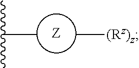
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
Ring Z is a ring selected from phenyl, naphthyl, a 5-10 membered heteroaryl containing 1-4 heteroatoms independently selected from nitrogen, oxygen, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
R^{u}, R^{v}, R^{w}, R^{x}, R^{y}, and R^{z} are, independently, hydrogen, halogen, C₁₋₆alkyl, C₁₋₆haloalkyl, —CN, —NO₂, —OR, —SR, —N(R)₂, —Si(R)₃, —S(O)₂R, —S(O)₂N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR,
—NRC(O)R, —NRC(O)N(R)₂, —NP(O)(R)₂, —NRP(O)(OR)₂, —NRP(O)(OR)N(R)₂, —NRP(O)(N(R)₂)₂, —NRS(O)₂R, or R^{A};
L^{x}, L^{y}, and L^{z} are, independently, a covalent bond or a bivalent, saturated or partially unsaturated, straight or branched C₁₋₅ hydrocarbon chain, wherein 0-3 methylene units of L^{x}, L^{y}, and L^{z} are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
s, s″, and s′″ are, independently, 0 or 1;
s′ is 1 or 2;
u, v, w, x, y, and z are, independently, 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, 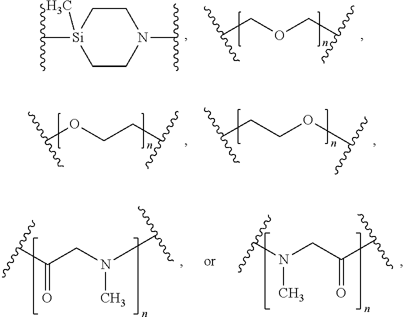
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R² is hydrogen, halogen, —CN, 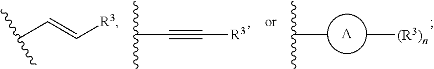
Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, halogen, C₁₋₆alkyl, C₁₋₆haloalkyl, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, —N(R)SO₂R, or R^{A}; or
two R³ groups are optionally taken together to form an optionally substituted 5-7 membered partially unsaturated or aryl fused ring having 0-2 heteroatoms independently selected from nitrogen, oxygen, and sulfur; and
n is 0, 1, 2, 4, or 5; or
A compound of formula I-bb-1: 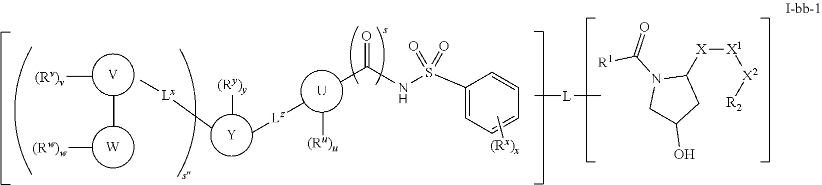
or a pharmaceutically acceptable salt, wherein:
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Ring W is a ring selected from phenyl, naphthyl, a 5-10 membered heteroaryl containing 1-4 heteroatoms independently selected from nitrogen, oxygen, selenium, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 4-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{u}, R^{v}, R^{w}, R^{x}, and R^{y} are, independently, hydrogen, halogen, C₁₋₆alkyl, C₁₋₆haloalkyl, —CN, —NO₂, —OR, —SR, —N(R)₂, —Si(R)₃, —S(O)₂R, —S(O)₂N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR,
—NRC(O)R, —NRC(O)N(R)₂, —NP(O)(R)₂, —NRP(O)(OR)₂, —NRP(O)(OR)N(R)₂, —NRP(O)(N(R)₂)₂, —NRS(O)₂R, or R^{A};
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x} and L^{y} are, independently, a covalent bond or a bivalent, saturated or partially unsaturated, straight or branched C₁₋₅ hydrocarbon chain, wherein 0-3 methylene units of L^{x} and L^{y} are independently replaced by a 4-6 membered carbocyclylenyl or heterocyclylenyl, optionally substituted 5-membered heteroarylenyl, —O—, —NR—, —CRF—, —CF₂—, —CROR—, —C(O)—, —S—, —S(O)—, or —S(O)₂—;
s and s″ are, independently, 0 or 1;
u, v, w, x, and y are, independently, 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, 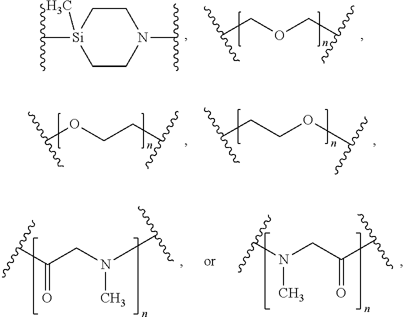
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)— or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R² is hydrogen, halogen, —CN 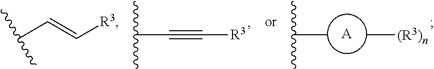
Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, halogen, C₁₋₆alkyl, C₁₋₆haloalkyl, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, —N(R)SO₂R, or R^{A}; or
two R³ groups are optionally taken together to form an optionally substituted 5-7 membered partially unsaturated or aryl fused ring having 0-2 heteroatoms independently selected from nitrogen, oxygen, and sulfur; and
n is 0, 1, 2, 4, or 5; or
A compound of formula I-cc-1: 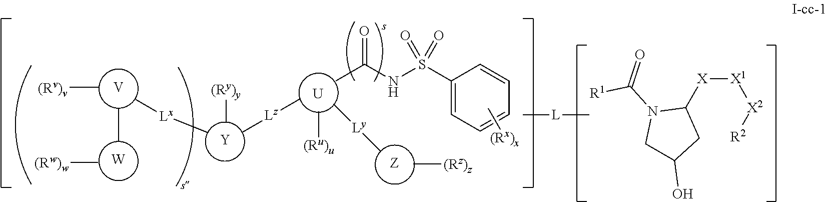
or a pharmaceutically acceptable salt, wherein:
Ring U is a bivalent ring selected from phenylenyl, a 5-15 membered saturated or partially unsaturated heterocyclylenyl with 1-4 heteroatoms independently selected from nitrogen, oxygen, and sulfur, and a 5-10 membered heteroarylenyl containing 1-4 heteroatoms independently selected from nitrogen, oxygen, and sulfur;
Ring V is a bivalent ring selected from a 5-6 membered saturated or partially unsaturated carbocyclylenyl or heterocyclylenyl with 1-4 heteroatoms independently selected from nitrogen, oxygen, and sulfur, or 5-6 membered heteroarylenyl with 1-4 heteroatoms independently selected from nitrogen, oxygen, and sulfur;
Ring W and Ring Z are, independently, a ring selected from phenyl, naphthyl, a 5-10 membered heteroaryl containing 1-4 heteroatoms independently selected from nitrogen, oxygen, selenium, and sulfur, and a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur;
Ring Y is a bivalent ring selected from phenylenyl, a 4-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur;
R^{u}, R^{v}, R^{w}, R^{x}, R^{y}, and R^{z}, are, independently, hydrogen, halogen, C₁₋₆alkyl, C₁₋₆haloalkyl, —CN, —NO₂, —OR, —SR, —N(R)₂, —Si(R)₃, —S(O)₂R, —S(O)₂N(R)₂, —S(O)₂NRC(O)R, —S(O)R, —S(O)₂OR, —C(O)R, —C(O)OR, —C(O)N(R)₂, —C(O)NROR, —C(O)NRC(O)R, —C(O)NRS(O)₂R, —OC(O)R, —OC(O)N(R)₂, —OP(O)(R)₂, —OP(O)(OR)₂, —OP(O)(OR)N(R)₂, —OP(O)(N(R)₂)₂, —NRC(O)OR,
—NRC(O)R, —NRC(O)N(R)₂, —NP(O)(R)₂, —NRP(O)(OR)₂, —NRP(O)(OR)N(R)₂, —NRP(O)(N(R)₂)₂, —NRS(O)₂R, or R^{A};
each R is independently hydrogen, or an optionally substituted group selected from C₁₋₆ aliphatic, phenyl, a 3-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form an optionally substituted 3-11 membered saturated or partially unsaturated monocyclic, bicyclic, bridged bicylic, or spirocyclic carbocyclic ring or heterocyclic ring with 1-4 heteroatoms independently selected from nitrogen, oxygen, and sulfur;
each R^{A} is independently an optionally substituted group selected from C₁₋₆ aliphatic, phenyl, a 3-10 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L^{x}, L^{y}, and L^{z} are, independently, a covalent bond or a bivalent, saturated or partially unsaturated, straight or branched C₁₋₅ hydrocarbon chain, wherein 0-3 methylene units of L^{x}, L^{Y}, and L^{z}, are independently replaced by a 4-6 membered carbocyclylenyl or heterocyclylenyl, optionally substituted 5-membered heteroarylenyl, —O—, —NR—, —CRF—, —CF₂−, —CROR—, —C(O)—, —S—, —S(O)—, or —S(O)₂—;
s and s″ are, independently, 0 or 1;
u, v, w, x, y, and z are, independently, 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —N(R)—, —Si(R)₂—, —Si(OH)(R)—, —Si(OH)₂—, —P(O)(OR)—, —P(O)(R)—, —P(O)(N(R)₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, 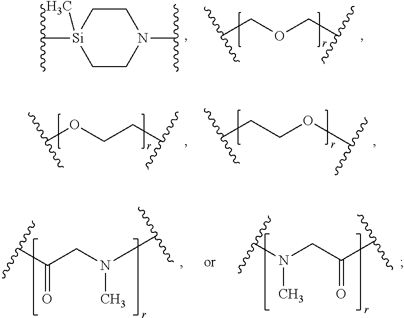
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10;
X is —C(O)—, —C(O)NR—, —SO₂—, —SO₂NR—, or an optionally substituted 5-membered heterocyclic ring;
X¹ is a bivalent group selected from a covalent bond, —O—, —C(O)—, —C(S)—, —C(R)₂—, —NR—, —S(O)—, or —SO₂—;
X² is an optionally substituted bivalent group selected from C₁₋₆ saturated or unsaturated alkylene, phenylenyl, a 5-6 membered heteroarylenyl containing 1-4 heteroatoms independently selected from nitrogen, oxygen, and sulfur, or a 4-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
R¹ is R^{A}, —C(R)₂R^{A}, —OR, —SR, —N(R)₂, —C(R)₂OR, —C(R)₂N(R)₂, —C(R)₂NRC(O)R, —C(R)₂NRC(O)N(R)₂, —NRC(O)OR, —NRC(O)R, —NRC(O)N(R)₂, or —NRSO₂R;
R² is hydrogen, halogen, —CN, 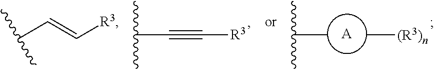
Ring A is a ring selected from phenyl, a 5-6 membered heteroaryl containing 1-4 heteroatoms independently selected from nitrogen, oxygen, and sulfur, or a 4 to 9-membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
each of R³ is independently hydrogen, halogen, C₁₋₆alkyl, C₁₋₆haloalkyl, —CN, —NO₂, —OR, —SR, —N(R)₂, —Si(R)₃, —SO₂R, —SO₂N(R)₂, —S(O)R, —C(O)R, —C(O)OR, —C(O)N(R)₂, —C(O)N(R)OR, —C(R)₂NRC(O)R, —C(R)₂NRC(O)N(R)₂, —OC(O)R, —OC(O)N(R)₂, —OP(O)(R)₂, —OP(O)(OR)₂, —OP(O)(OR)N(R)₂, —OP(O)(N(R)₂)₂—, —N(R)C(O)OR, —N(R)C(O)R, —NRC(O)N(R)₂, —N(R)SO₂R, —NP(O)(R)₂, —N(R)P(O)(OR)₂, —N(R)P(O)(OR)N(R)₂, —N(R)P(O)(N(R)₂)₂, —N(R)SO₂R, or R^{A}; or
two R³ groups are optionally taken together to form an optionally substituted 5-7 membered partially unsaturated or aryl fused ring having 0-2 heteroatoms independently selected from nitrogen, oxygen, and sulfur; and
n is 0, 1, 2, 4, or 5.

2. The compound of claim 1, wherein the compound is a compound of any one of the following formulae: 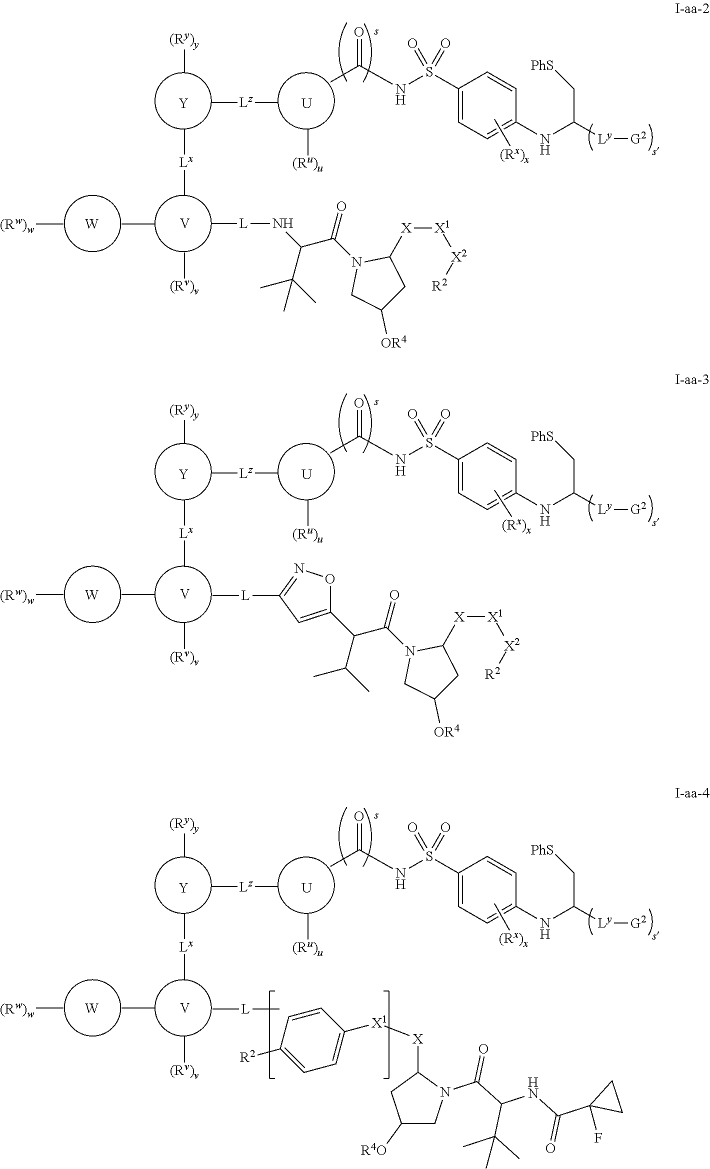 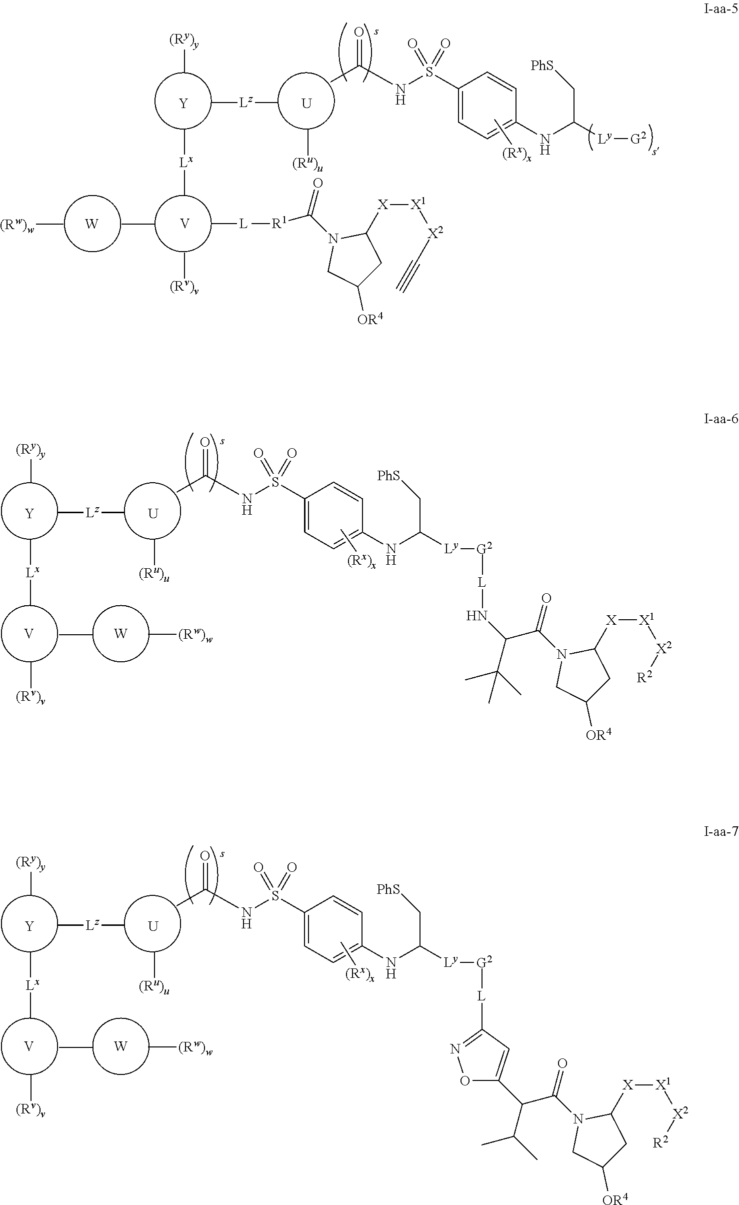 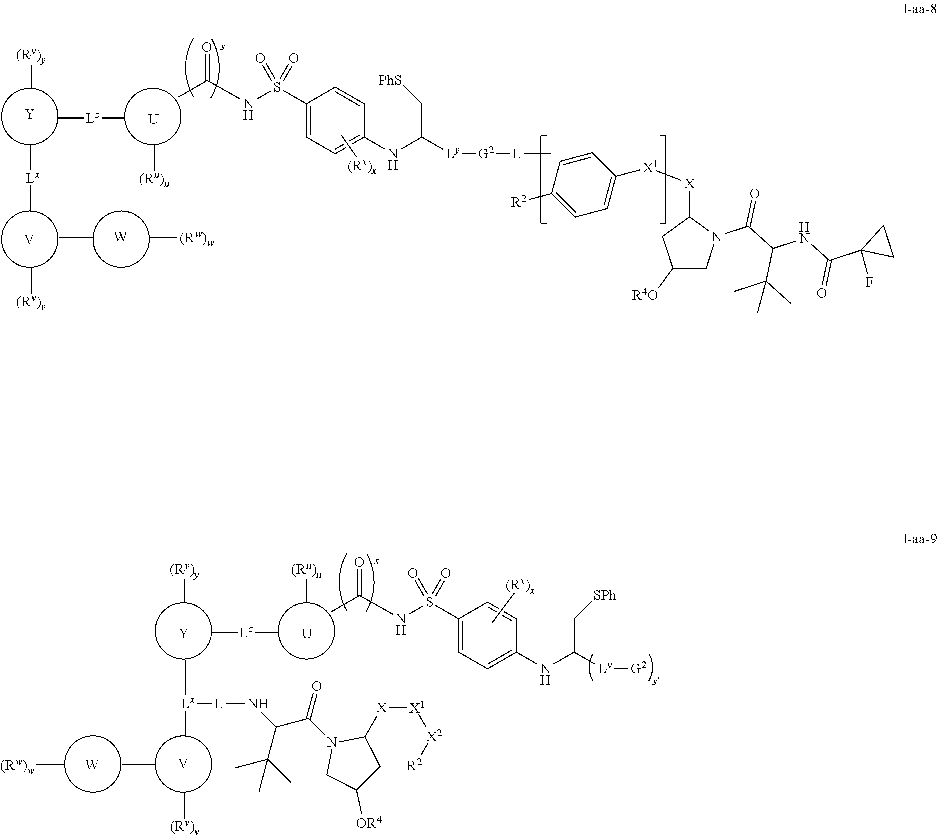
or a pharmaceutically acceptable salt thereof.

3. (canceled)

4. The compound of claim 1, wherein the compound is a compound of any one of the following formulae: 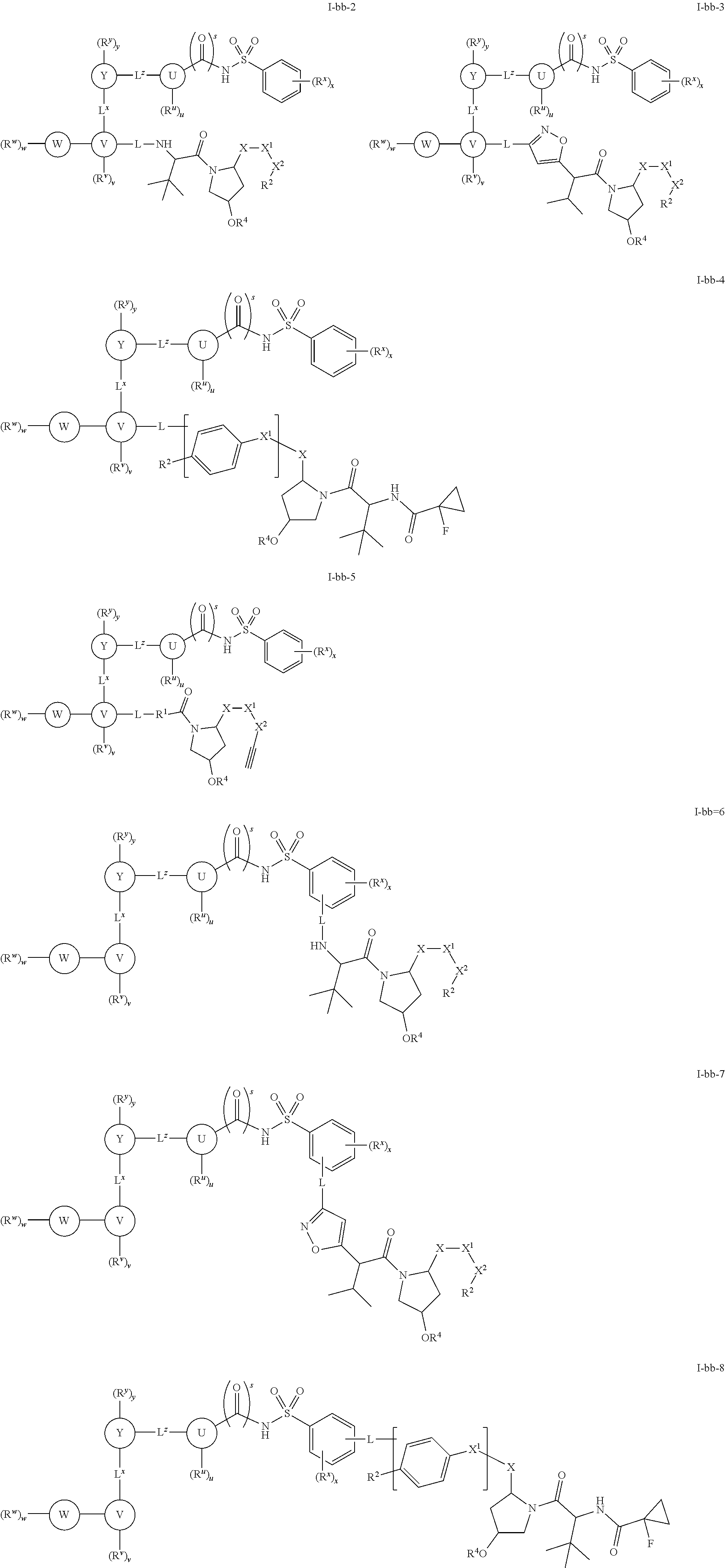
or a pharmaceutically acceptable salt thereof.

5. (canceled)

6. The compound of claim 1, wherein the compound is a compound of any one of the following formulae: 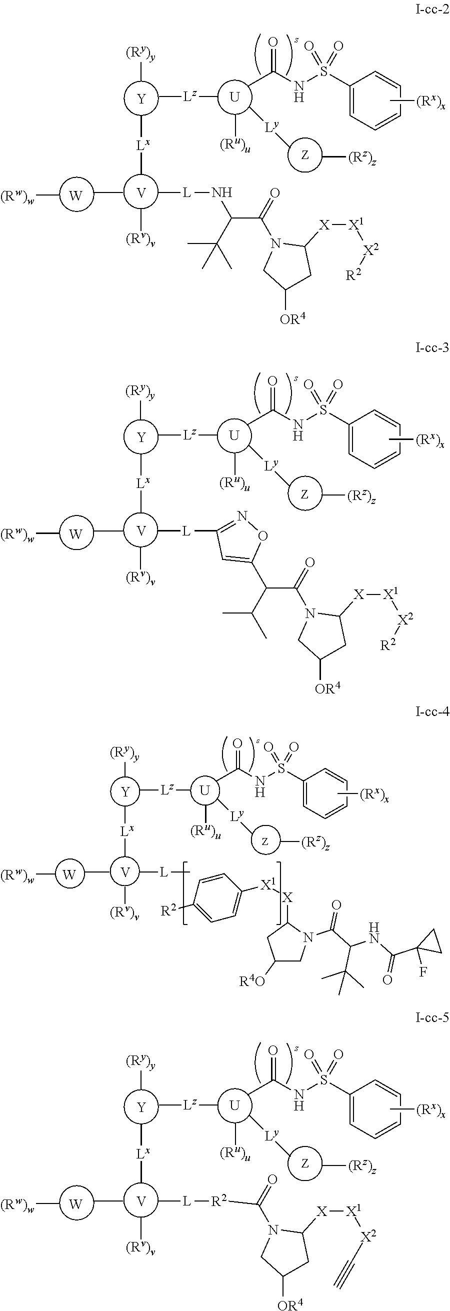 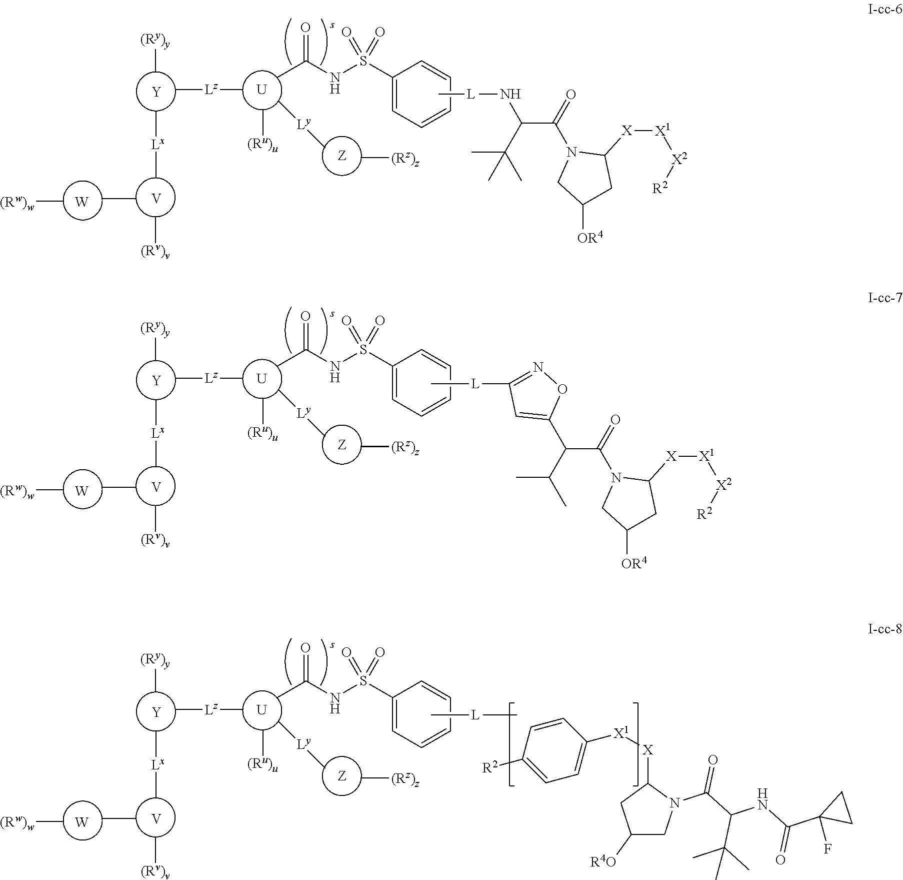
or a pharmaceutically acceptable salt thereof.

7. The compound of claim 1, wherein Ring W is phenyl or a 5-10 membered heteroaryl containing 1-4 heteroatoms independently selected from nitrogen, oxygen, and sulfur.

8. The compound of claim 1, wherein Ring Y is a 4-11 membered saturated or partially unsaturated monocyclic, bicyclic, bridged bicyclic, or spirocyclic carbocyclylenyl or heterocyclylenyl with 1-4 heteroatoms independently selected from nitrogen, oxygen, and sulfur, and a 5-6 membered heteroarylenyl containing 1-4 heteroatoms independently selected from nitrogen, oxygen, and sulfur.

9. The compound of claim 1, wherein Ring Z is a 5-10 membered heteroaryl containing 1-4 heteroatoms independently selected from nitrogen, oxygen, selenium, and sulfur, or a 5-11 membered saturated or partially unsaturated monocyclic, bicyclic, bridged bicylic, or spirocyclic carbocyclyl or heterocyclyl with 1-4 heteroatoms independently selected from nitrogen, oxygen, and sulfur.

10. The compound of any one of claims 1-9, wherein L is a covalent bond or a bivalent, saturated or partially unsaturated, straight or branched C₁-20 hydrocarbon chain, wherein 0-6 methylene units of L are independently replaced by -Cy-, —O—, —N(R)—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, or —N(R)C(O)O—.

11. The compound of claim 1, wherein X is —C(O)NR— or an optionally substituted 5-membered heterocyclic ring.

12. The compound of claim 1, wherein X¹ is a covalent bond, —O—, —CH₂—, or —CHMe—.

13. The compound of claim 1, wherein X² is phenylenyl or a 5-6 membered heteroarylenyl containing 1-4 heteroatoms independently selected from nitrogen, oxygen, and sulfur.

14. The compound of claim 1, wherein R² is

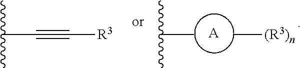

15. The compound of claim 1, wherein Ring A is a 5-6 membered heteroaryl containing 1-4 heteroatoms independently selected from nitrogen, oxygen, and sulfur.

16. The compound of claim 1, wherein said compound is selected from any one of the compounds depicted in Table 1, or a pharmaceutically acceptable salt thereof.

17. A pharmaceutical composition comprising a compound according to claim 1, and a pharmaceutically acceptable carrier, adjuvant, or vehicle.

18. The pharmaceutical composition according to claim 17, further comprising an additional therapeutic agent.

19. A method of degrading BCL-XL and BCL-2 protein in a patient or biological sample comprising administering to said patient or contacting said biological sample with a compound according to claim 1, or a pharmaceutical composition thereof.

20. A method of treating an BCL-XL and BCL-2 mediated disorder, disease, or condition in a patient comprising administering to said patient a compound according to claim 1, or a pharmaceutical composition thereof.

21. (canceled)

22. The method of claim 20, wherein the BCL-XL and BCL-2 mediated disorder, disease or condition is a cancer, an autoimmune disease, or inflammation.

23. The method of claim 22, wherein the cancer is selected from synovial sarcoma, Burkitt lymphoma, Hodgkin lymphoma, multiple myeloma, neuroblastoma, glioblastoma, small cell lung cancer, pancreatic cancer, hepatocellular (liver) cancer, endometrial cancer, ovarian cancer, cervical cancer, breast cancer, prostate cancer, bladder cancer, melanoma, rhabdomyosarcoma, osteosarcoma/malignant fibrous histiocytoma of bone, choriocarcinoma, kidney cancer (renal cell cancer), thyroid cancer, and leukemias (acute lymphoblastic, acute myeloid, chronic lymphocytic, and chronic myelogenous).
